(19) United States
(12) Patent Application Publication
(10) Pub. No.: US 2024/0279202 A1
(43) Pub. Date: Aug. 22, 2024

(54) COMPOUNDS FOR INHIBITING OR DEGRADING ITK, COMPOSITIONS, COMPRISING THE SAME METHODS OF THEIR MAKING AND METHODS OF THEIR USE

(71) Applicant: NURIX THERAPEUTICS, INC., San Francisco, CA (US)

(72) Inventors: Joel MCINTOSH, San Francisco, CA (US); Daisuke KATO, San Francisco, CA (US); Jeffrey MIHALIC, San Francisco, CA (US); Ge PENG, San Francisco, CA (US)

(21) Appl. No.: 18/289,513

(22) PCT Filed: May 3, 2022
(86) PCT No.: PCT/US2022/027532

§ 371 Date: Nov. 3, 2023

Related U.S. Application Data
(60) Provisional application No. 63/183,617, filed on May 3, 2021.

(51) Int. Cl. C07D 401/14; A61K 31/454; A61K 31/4545; A61K 31/4709; A61K 31/4725; A61K 31/496; A61K 31/513; C07D 471/10

(52) U.S. Cl. CPC C07D 401/14; A61K 31/454; A61K 31/4545; A61K 31/4709; A61K 31/4725; A61K 31/496; A61K 31/513; C07D 471/10

(57) ABSTRACT

Provided herein are compounds for inhibiting and/or degrading IL-2 inducible T-cell kinase (ITK), compositions comprising the compounds, methods of making the same, and methods of their use for treating diseases or disorders including cancer, inflammatory, and autoimmune diseases.

### CROSS-REFERENCE TO RELATED APPLICATIONS

This application is a PCT International Application, claiming the benefit of U.S. Provisional Application No. 63/183,617, filed on May 3, 2021, the entire content of which is herein incorporated by reference.

### FIELD

Provided herein are compounds for inhibiting and/or degrading IL-2 inducible T-cell kinase, compositions comprising the compounds, methods of making the same, and methods of their use for treating diseases or disorders including cancer, inflammatory, and autoimmune diseases.

### BACKGROUND

IL-2 inducible T-cell kinase (ITK) is a tyrosine protein kinase and a member of the TEC family of kinases. Gibson et al., 1993, Blood 82(5):1561-1572. ITK is highly expressed in T-cells. Gomez-Rodriguez et al., 2011, FEBS J. 278(12):1980-1989. ITK is reported to be activated downstream of the T-cell receptor (TCR) via phosphorylation from Lck kinase. Gomez-Rodriguez et al., 2011. ITK is believed to activate phospholipase Cγ1 (PLCγ1) to drive T-cell function and immune responses. Gomez-Rodriguez et al., 2011; Kosaka et al., 2006, Trends Immunol. 27(10):453-60. ITK has been shown to be involved in numerous inflammatory, autoimmune, and proliferative diseases including allergic asthma, atopic dermatitis, aplastic anemia, inflammatory bowel disease, neuroinflammation, and T cell lymphomas. Lechner et al., 2020, J. Mol. Medicine 98:1385-1395. ITK provides a promising target for therapies for treating several inflammatory, autoimmune, and proliferative diseases and disorders.

### SUMMARY

Provided herein are compounds capable of binding, inhibiting, and/or degrading ITK. The compounds are useful for the treatment or prevention of inflammatory, autoimmune, and proliferative diseases and disorders in a subject in need thereof.

In a first aspect, provide herein are compounds according to Formula (A):

ITK Hook−Linker−Ubiquitin Ligase Harness (A)

The compounds comprise an ITK hook. The ITK hook is a moiety capable of binding ITK in vitro, in vivo, and/or in a cell. Useful ITK hooks are described herein. The ubiquitin ligase harness is a moiety capable of harnessing a ubiquitin ligase in vitro, in vivo, and/or in a cell. In certain embodiments, the ubiquitin ligase is an E3 ligase. In certain embodiments, the ubiquitin ligase is cereblon. Useful ubiquitin ligase harnesses are described herein. The compounds further comprise a linker. The linker is any moiety capable of covalently binding the harness and the hook while permitting each to bind or harness its target. By harnessing a ubiquitin ligase and binding ITK, the compounds are capable of targeting ITK for degradation under the appropriate conditions, for instance in a cell. As shown in the Examples herein, the compounds degrade ITK in splenocytes and in vivo. Degrading ITK provides a mechanism useful for treating inflammatory, autoimmune, and proliferative diseases and disorders in subjects in need thereof.

In one aspect, provided herein are compounds of Formula (I), or stereoisomers and pharmaceutically acceptable salt(s) thereof:

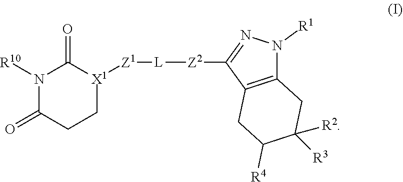

The left side of the molecule is a ubiquitin ligase harness. The right side of the molecule is an ITK hook. The middle portion of the molecule is a linker. The compounds are described in detail herein.

In another aspect, provided herein are pharmaceutical compositions. The pharmaceutical compositions comprise the compounds along with one or more pharmaceutically acceptable carriers, diluents, or excipients.

In another aspect, provided herein are methods of treating or preventing a disease or disorder in a subject in need thereof. In certain embodiments, the disease or disorder is an inflammatory disease or disorder. In certain embodiments, the disease or disorder is an autoimmune disease or disorder. In certain embodiments, the disease or disorder is a proliferative disease or disorder, for instance a T-cell lymphoma. In another aspect, provided herein are the compounds and compositions for use in therapy. In another aspect, provided herein are the compounds and compositions for use in the treatment or prevention of inflammatory, autoimmune, or proliferative diseases and disorders. In another aspect, provided herein are the uses of the compounds and compositions for the manufacture of medicaments. In another aspect, provided herein are the uses of the compounds and compositions for the manufacture of medicaments for the treatment or prevention of treatment or prevention of inflammatory, autoimmune, or proliferative diseases and disorders.

In another aspect, provided herein are methods of making the compounds or compositions. Synthetic procedures for their preparation are described in detail herein.

The compounds and compositions are useful for binding, inhibiting, and/or degrading ITK in vitro, in vivo, and/or in a cell. As such, the compounds and compositions are useful for the treatment or prevention of treatment or prevention of inflammatory, autoimmune, or proliferative diseases and disorders.

### BRIEF DESCRIPTION OF THE FIGURES

FIG. 1 provides in vitro degradation of ITK in a HiBiT cell line.

FIG. 2 provides in vivo degradation of ITK in mouse splenocytes.

FIG. 3A provides in vivo degradation of ITK in mouse splenocytes following administration of compounds provided herein observed by Western blotting; FIG. 3B provides graphs of ITK levels in mouse splenocytes following administration of compounds provided herein.

FIG. 4A provides in vivo degradation of ITK in mouse splenocytes following administration of compounds provided herein observed by Western blotting; FIG. 4B provides graphs of ITK levels in mouse splenocytes following administration of compounds provided herein; FIG. 4C provides compound concentration over time in vivo.

### DETAILED DESCRIPTION OF THE PREFERRED EMBODIMENTS

Provided herein are compounds for inhibiting and/or degrading IL-2 inducible T-cell kinase, compositions comprising the compounds, methods of making the same, and methods of their use for treating diseases or disorders including cancer, inflammatory, and autoimmune diseases.

### Definitions

For purposes herein, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

As described herein, compounds herein optionally may be substituted with one or more substituents, such as those illustrated generally herein, or as exemplified by particular classes, subclasses, and species of the description.

As used herein, the term “hydroxyl” or “hydroxy” refers to an —OH moiety.

As used herein, the term “aliphatic” encompasses the terms alkyl, alkenyl, and alkynyl, each of which are optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-4 or 2-6) carbon atoms and at least one double bond. Like an alkyl group, an alkenyl group can be straight or branched. Examples of an alkenyl group include, but are not limited to, allyl, 1- or 2-isopropenyl, 2-butenyl, and 2-hexenyl. An alkenyl group can be optionally substituted with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-4 or 2-6) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino, or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino.” These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl, or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen (H or —H), aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-SO₂— or amino-SO₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl (e.g., mono-, di-(such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfamoyl)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl), cycloaliphatic (e.g., cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9-, 10-, or 11-) membered structures that form two rings, wherein the two rings have at least one atom in common (e.g., two atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which are optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino), nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), cyano, halo, hydroxy, mercapto, sulfonyl (e.g., alkyl-SO₂— and aryl-SO₂—), sulfinyl (e.g., alkyl-S(O)—), sulfanyl (e.g., alkyl-S—), sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused, bridged, or spiro) (e.g., 5- to 10-membered mono- or bicyclic) saturated ring structure, in which one or more of the ring atoms is a heteroatom (e.g., nitrogen (N), oxygen (O), sulfur (S), or combinations thereof). Non-limiting examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholinyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, decahydro-2,7-naphthyridine, 2,8-diazaspiro[4.5]decane, 2,7-diazaspiro[3.5]nonane, octahydropyrrolo[3,4-c]pyrrole, octahydro-1H-pyrrolo[3,4-b]pyridine, and 2,6-dioxa-tricyclo[3.3.1.0^{3,7}]nonyl. A monocyclic heterocycloalkyl group can be fused with a phenyl moiety to form structures, such as tetrahydroisoquinoline, that would be categorized as heteroaryls.

A “heterocycloalkenyl” group, as used herein, refers to a mono- or bicylic (e.g., 5 to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring system having four to fifteen ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having two to three rings. For example, a benzofused group includes benzo fused with one or two 4-to 8-membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine.

Without limitation, monocyclic heteroaryls include furyl, thiophene-yl, 2H-pyrrolyl, oxazolyl, thiazolyl, imidazolyl, pyrazolyl, isoxazolyl, isothiazolyl, 1,3,4-thiadiazolyl, 2H-pyranyl, 4H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphaticsulfonyl or aminosulfonyl); sulfinyl (e.g., aliphaticsulfinyl); sulfanyl (e.g., aliphaticsulfanyl); nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl; or (haloalkyl)heteroaryl (e.g., trihaloalkylheteroaryl).

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined herein.

As used herein, an “alkoxy” group refers to an alkyl-O— group where “alkyl” has been defined previously herein.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, when used as a terminal group; or —OC(O)—, or —C(O)O— when used as an internal group.

As used herein, an ester refers to —COOR^{X} when used as a terminal group; or —COOR^{X}— when used as an internal group, wherein R^{X} has been defined above.

As used herein, a formate refers to —OC(O)H.

As used herein, an acetate refers to —OC(O)R^{X}, wherein R^{X} has been defined above.

As used herein, a “haloaliphatic” group refers to an aliphatic group substituted with one to three halogen. For instance, the term haloalkyl includes the group —CF₃.

As used herein, a “mercapto” or “sulfhydryl” group refers to —SH.

As used herein, a “sulfo” group refers to —SO₃H, or —SO₃R^{X} when used terminally or S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y}, and R^{Z} have been defined above.

As used herein, a “sulfonamide” group refers to the structure —S(O)₂—NR^{X}R^{Y}, or —NR^{X}—S(O)₂—R^{Z} when used terminally; or —S(O)₂—NR^{X}—, or —NR^{X}—S(O)₂— when used internally, wherein R^{X}, R^{Y}, and R^{Z} are defined above.

As used herein a “sulfanyl” group refers to —S—R^{X} when used terminally and —S-when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Examples of sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, and/or the like.

As used herein, a “sulfonyl” group refers to-S(O)₂—R^{X} when used terminally and —S(O)₂— when used internally, wherein R^{X} has been defined above. Examples of sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂—, and/or the like.

As used herein, a “sulfoxy” group refers to —O—S(O)—R^{X}, or —S(O)—O—R^{X}, when used terminally and —O—S(O)—, or —S(O)—O— when used internally, where R^{X} has been defined above.

As used herein, a “halogen” or “halo” group refers to fluorine (F), chlorine (Cl), bromine (Br), or iodine (I).

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates.

Examples of phosphinates and phosphonates include —P(O)(RP)₂, wherein R^{P} is aliphatic, alkoxy, aryloxy, heteroaryloxy, (cycloaliphatic)oxy, (heterocycloaliphatic)oxy, aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} each when used terminally and —NR^{X}—CO—NR^{Y}— or —NR^{X}—CS—NR^{X}— each when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl (i.e., R^{X}O(O)C-alkyl) is an example of a carboxy group used terminally. A group is internal when the group is present in the middle of or within the termini of a substituent of the chemical structure. Alkylcarboxy (e.g., alkyl-C(O)O— or alkyl-OC(O)—) and alkylcarboxyaryl (e.g., alkyl-C(O)O-aryl-, or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ-, where each Q is independently a hydrogen (H or —H), or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used herein interchangeably with the phrase “substituted or unsubstituted.” As described herein, compounds herein can optionally be substituted with one or more substituents, such as are illustrated generally above, or as exemplified by particular classes, subclasses, and species of the description. As described herein, the variables R, R¹, R², L, and Z¹, and other variables contained in Formula (I), (II), (III), (IIIa), (IV), (IVa), (V), (Va), (Vb), (VI), (VIa), and (VIb), described herein encompass specific groups, such as alkyl and aryl. Unless otherwise noted, each of the specific groups for the variables R, R¹⁰, R^{A}, R¹, R², L, L¹, D, W, E, V, G, Y, and Z, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, for example, both rings share one common atom. Non-limiting examples of spiro heterocycloalkyls include

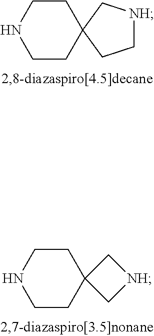 [SMILES: C1CC2(CCN1)CCNC2 ; C1CC2(CCN1)CNC2]

Spiro compounds depicted with overlapping rings indicate that the rings can bond at any vertex. For instance, in the spiro group

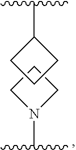

the two rings can bond at any of the three available vertex atoms in either ring.

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, and their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile.

The term “excipients” as used herein include pharmaceutically acceptable excipients, carriers, vehicles or stabilizers that are nontoxic to the cell or mammal being exposed thereto at the dosages and concentrations employed. In certain embodiments, the physiologically acceptable excipient is an aqueous pH buffered solution.

The terms “treating” or “treatment” of a disease refer to executing a protocol, which may include administering one or more therapeutic agent to an individual (human or otherwise), in an effort to obtain beneficial or desired results in the individual, including clinical results. In certain embodiments beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total). In certain embodiments, “treatment” also can mean prolonging survival as compared to expected survival of an individual not receiving treatment. In certain embodiments, “treating” and “treatment” may occur by administration of one dose of a therapeutic agent or therapeutic agents, or may occur upon administration of a series of doses of a therapeutic agent or therapeutic agents. In certain embodiments, “treating” or “treatment” does not require complete alleviation of signs or symptoms, and does not require a cure. In certain embodiments, “treatment” also can refer to clinical intervention, such as administering one or more therapeutic agents to an individual, designed to alter the natural course of the individual or cell being treated (i.e., to alter the course of the individual or cell that would occur in the absence of the clinical intervention). In certain embodiments, the term “therapeutic agent” can refer to a CTM drug that induce the proteolytic degradation of tyrosine-protein kinase (ITK), or compositions thereof.

The term an “individual” a “patient” or a “subject” refers a mammal. In certain embodiments, a “mammal” for purposes of treatment includes humans; non-human primates; domestic and farm animals; and zoo, sports, or pet animals, such as dogs, horses, rabbits, cattle, pigs, hamsters, gerbils, mice, ferrets, rats, cats, etc. In some embodiments, the individual or subject is human.

As used herein, the term “about” means within +10% of a value. For example, a dose that is about 100 mg/kg provides that the dose can be 90 mg/kg to 110 mg/kg. By way of further example, an amount of an additional therapeutic agent ranging from about 50% to about 100% provides that the amount of additional therapeutic agent ranges from 45-55% to 90-110%. A person of skill in the art will appreciate the scope and application of the term “about” when used to describe other values disclosed herein.

Unless otherwise stated, structures depicted herein also are meant to include all isomeric (e.g., enantiomeric, diastereomeric, and geometric (or conformational)) forms of the structure; for example, the (R)- and (S)-configurations for each asymmetric center, (Z)- and (E)-double bond isomers, and (Z)- and (E)-conformational isomers. Therefore, single stereochemical isomers as well as enantiomeric, diastereomeric, and geometric (or conformational) mixtures of the present compounds are within the scope of the description. Alternatively, as used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]-[(S)-compound])/([(R)-compound]+[(S)-compound])×100, where the (R)-compound>(S)-compound; or % ee=([(S)-compound]-[(R)-compound])/([(S)-compound]+[(R)-compound])×100, where the (S)-compound>(R)-compound. Moreover, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, the compounds or inhibitors described herein have an ee, de, % ee, or % de greater than zero. For example, in certain embodiments, the compounds or inhibitors described herein have an ee, de, % ee, or % de of ten. In certain embodiments, the compounds or inhibitors described herein have an ee, de, % ee, or % de of twenty-five. In certain embodiments, the compounds or inhibitors described herein have an ee, de, % ee, or % de of fifty. In certain embodiments, the compounds or inhibitors described herein have an ee, de, % ee, or % de of seventy-five.

In certain embodiments, the compounds or inhibitors described herein have an ee, de, % ee, or % de range from ninety to one hundred. In certain embodiments, the compounds or inhibitors described herein have an ee, de, % ee, or % de range from ninety-five to one hundred. In certain embodiments, the compounds or inhibitors described herein have an ee, de, % ee, or % de range from ninety-seven to one hundred. In certain embodiments, the compounds or inhibitors described herein have an ee, de, % ee, or % de range from ninety-eight to one hundred. In certain embodiments, the compounds or inhibitors described herein have an ee, de, % ee, or % de range from ninety-nine to one hundred.

In one embodiment of a compound or inhibitor described herein, the ee, de, % ee, or % de is one. In one embodiment of a compound or inhibitor described herein, the ee, de, % ee, or % de is two. In one embodiment of a compound or inhibitor described herein, the ee, de, % ee, or % de is three. In one embodiment of a compound or inhibitor described herein, the ee, de, % ee, or % de is four. In one embodiment of a compound or inhibitor described herein, the ee, de, % ee, or % de is five. In one embodiment of a compound or inhibitor described herein, the ee, de, % ee, or % de is six. In one embodiment of a compound or inhibitor described herein, the ee, de, % ee, or % de is seven. In one embodiment of a compound or inhibitor described herein, the ee, de, % ee, or % de is eight. In one embodiment of a compound or inhibitor described herein, the ee, de, % ee, or % de is nine. In one embodiment of a compound or inhibitor described herein, the ee, de, % ee, or % de is ten. In one embodiment of a compound or inhibitor described herein, the ee, de, % ee, or % de is eleven. In one embodiment of a compound or inhibitor described herein, the ee, de, % ee, or % de is twelve. In one embodiment of a compound or inhibitor described herein, the ee, de, % ee, or % de is thirteen. In one embodiment of a compound or inhibitor described herein, the ee, de, % ee, or % de is fourteen. In one embodiment of a compound or inhibitor described herein, the ee, de, % ee, or % de is fifteen. In one embodiment of a compound or inhibitor described herein, the ee, de, % ee, or % de is sixteen. In one embodiment of a compound or inhibitor described herein, the ee, de, % ee, or % de is seventeen. In one embodiment of a compound or inhibitor described herein, the ee, de, % ee, or % de is eighteen. In one embodiment of a compound or inhibitor described herein, the ee, de, % ee, or % de is nineteen. In one embodiment of a compound or inhibitor described herein, the ee, de, % ee, or % de is twenty. In one embodiment of a compound or inhibitor described herein, the ee, de, % ee, or % de is twenty-one. In one embodiment of a compound or inhibitor described herein, the ee, de, % ee, or % de is twenty-two. In one embodiment of a compound or inhibitor described herein, the ee, de, % ee, or % de is twenty-three. In one embodiment of a compound or inhibitor described herein, the ee, de, % ee, or % de is twenty-four. In one embodiment of a compound or inhibitor described herein, the ee, de, % ee, or % de is twenty-five. In one embodiment of a compound or inhibitor described herein, the ee, de, % ee, or % de is twenty-six. In one embodiment of a compound or inhibitor described herein, the ee, de, % ee, or % de is twenty-seven. In one embodiment of a compound or inhibitor described herein, the ee, de, % ee, or % de is twenty-eight. In one embodiment of a compound or inhibitor described herein, the ee, de, % ee, or % de is twenty-nine. In one embodiment of a compound or inhibitor described herein, the ee, de, % ee, or % de is thirty. In one embodiment of a compound or inhibitor described herein, the ee, de, % ee, or % de is thirty-one. In one embodiment of a compound or inhibitor described herein, the ee, de, % ee, or % de is thirty-two. In one embodiment of a compound or inhibitor described herein, the ee, de, % ee, or % de is thirty-three. In one embodiment of a compound or inhibitor described herein, the ee, de, % ee, or % de is thirty-four. In one embodiment of a compound or inhibitor described herein, the ee, de, % ee, or % de is thirty-five. In one embodiment of a compound or inhibitor described herein, the ee, de, % ee, or % de is thirty-six. In one embodiment of a compound or inhibitor described herein, the ee, de, % ee, or % de is thirty-seven. In one embodiment of a compound or inhibitor described herein, the ee, de, % ee, or % de is thirty-eight. In one embodiment of a compound or inhibitor described herein, the ee, de, % ee, or % de is thirty-nine. In one embodiment of a compound or inhibitor described herein, the ee, de, % ee, or % de is forty. In one embodiment of a compound or inhibitor described herein, the ee, de, % ee, or % de is forty-one. In one embodiment of a compound or inhibitor described herein, the ee, de, % ee, or % de is forty-two. In one embodiment of a compound or inhibitor described herein, the ee, de, % ee, or % de is forty-three. In one embodiment of a compound or inhibitor described herein, the ee, de, % ee, or % de is forty-four. In one embodiment of a compound or inhibitor described herein, the ee, de, % ee, or % de is forty-five. In one embodiment of a compound or inhibitor described herein, the ee, de, % ee, or % de is forty-six. In one embodiment of a compound or inhibitor described herein, the ee, de, % ee, or % de is forty-seven. In one embodiment of a compound or inhibitor described herein, the ee, de, % ee, or % de is forty-eight. In one embodiment of a compound or inhibitor described herein, the ee, de, % ee, or % de is forty-nine. In one embodiment of a compound or inhibitor described herein, the ee, de, % ee, or % de is fifty. In one embodiment of a compound or inhibitor described herein, the ee, de, % ee, or % de is fifty-one. In one embodiment of a compound or inhibitor described herein, the ee, de, % ee, or % de is fifty-two. In one embodiment of a compound or inhibitor described herein, the ee, de, % ee, or % de is fifty-three. In one embodiment of a compound or inhibitor described herein, the ee, de, % ee, or % de is fifty-four. In one embodiment of a compound or inhibitor described herein, the ee, de, % ee, or % de is fifty-five. In one embodiment of a compound or inhibitor described herein, the ee, de, % ee, or % de is fifty-six. In one embodiment of a compound or inhibitor described herein, the ee, de, % ee, or % de is fifty-seven. In one embodiment of a compound or inhibitor described herein, the ee, de, % ee, or % de is fifty-eight. In one embodiment of a compound or inhibitor described herein, the ee, de, % ee, or % de is fifty-nine. In one embodiment of a compound or inhibitor described herein, the ee, de, % ee, or % de is sixty. In one embodiment of a compound or inhibitor described herein, the ee, de, % ee, or % de is sixty-one. In one embodiment of a compound or inhibitor described herein, the ee, de, % ee, or % de is sixty-two. In one embodiment of a compound or inhibitor described herein, the ee, de, % ee, or % de is sixty-three. In one embodiment of a compound or inhibitor described herein, the ee, de, % ee, or % de is sixty-four. In one embodiment of a compound or inhibitor described herein, the ee, de, % ee, or % de is sixty-five. In one embodiment of a compound or inhibitor described herein, the ee, de, % ee, or % de is sixty-six. In one embodiment of a compound or inhibitor described herein, the ee, de, % ee, or % de is sixty-seven. In one embodiment of a compound or inhibitor described herein, the ee, de, % ee, or % de is sixty-eight. In one embodiment of a compound or inhibitor described herein, the ee, de, % ee, or % de is sixty-nine. In one embodiment of a compound or inhibitor described herein, the ee, de, % ee, or % de is seventy. In one embodiment of a compound or inhibitor described herein, the ee, de, % ee, or % de is seventy-one. In one embodiment of a compound or inhibitor described herein, the ee, de, % ee, or % de is seventy-two. In one embodiment of a compound or inhibitor described herein, the ee, de, % ee, or % de is seventy-three. In one embodiment of a compound or inhibitor described herein, the ee, de, % ee, or % de is seventy-four. In one embodiment of a compound or inhibitor described herein, the ee, de, % ee, or % de is seventy-five. In one embodiment of a compound or inhibitor described herein, the ee, de, % ee, or % de is seventy-six. In one embodiment of a compound or inhibitor described herein, the ee, de, % ee, or % de is seventy-seven. In one embodiment of a compound or inhibitor described herein, the ee, de, % ee, or % de is seventy-eight. In one embodiment of a compound or inhibitor described herein, the ee, de, % ee, or % de is seventy-nine. In one embodiment of a compound or inhibitor described herein, the ee, de, % ee, or % de is eighty. In one embodiment of a compound or inhibitor described herein, the ee, de, % ee, or % de is eighty-one. In one embodiment of a compound or inhibitor described herein, the ee, de, % ee, or % de is eighty-two. In one embodiment of a compound or inhibitor described herein, the ee, de, % ee, or % de is eighty-three. In one embodiment of a compound or inhibitor described herein, the ee, de, % ee, or % de is eighty-four. In one embodiment of a compound or inhibitor described herein, the ee, de, % ee, or % de is eighty-five. In one embodiment of a compound or inhibitor described herein, the ee, de, % ee, or % de is eighty-six. In one embodiment of a compound or inhibitor described herein, the ee, de, % ee, or % de is eighty-seven. In one embodiment of a compound or inhibitor described herein, the ee, de, % ee, or % de is eighty-eight. In one embodiment of a compound or inhibitor described herein, the ee, de, % ee, or % de is eighty-nine. In one embodiment of a compound or inhibitor described herein, the ee, de, % ee, or % de is ninety. In one embodiment of a compound or inhibitor described herein, the ee, de, % ee, or % de is ninety-one. In one embodiment of a compound or inhibitor described herein, the ee, de, % ee, or % de is ninety-two. In one embodiment of a compound or inhibitor described herein, the ee, de, % ee, or % de is ninety-three. In one embodiment of a compound or inhibitor described herein, the ee, de, % ee, or % de is ninety-four. In one embodiment of a compound or inhibitor described herein, the ee, de, % ee, or % de is ninety-five. In one embodiment of a compound or inhibitor described herein, the ee, de, % ee, or % de is ninety-six. In one embodiment of a compound or inhibitor described herein, the ee, de, % ee, or % de is ninety-seven. In one embodiment of a compound or inhibitor described herein, the ee, de, % ee, or % de is ninety-eight. In one embodiment of a compound or inhibitor described herein, the ee, de, % ee, or % de is ninety-nine. In one embodiment of a compound or inhibitor described herein, the ee, de, % ee, or % de is one hundred. In certain embodiments, compounds or inhibitors described within Table 1 herein have an ee, de, % ee, or % de as described within this paragraph. In certain embodiments, compounds or inhibitors described in the Examples and/or Biological Examples have an ee, de, % ee, or % de as described within this paragraph. Unless otherwise stated, all tautomeric forms of the compounds of the description are within the scope of the description. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this description. Such compounds are useful, for example, as analytical tools or probes in biological assays, or as therapeutic agents.

As used herein, the term “& 1” means that a compound including the “& 1” notation at a particular chemical element or atom (e.g., carbon) within the compound was prepared as a mixture of two stereoisomers at the noted chemical element or atom (e.g., a diastereomeric mixture having a de or % de as described above).

Chemical structures and nomenclature are derived from ChemDraw, version 19.0, Cambridge, MA.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

### Compounds

In one aspect, provided herein are compounds of Formula (I), and stereoisomers and pharmaceutically acceptable salt(s) thereof:

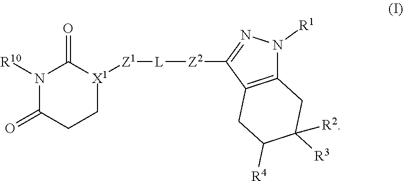

In Formula (I), R¹ is hydrogen or C₁₋₃ alkyl; R² is hydrogen, substituted or unsubstituted C₁₋₅ alkyl, or substituted or unsubsituted C₁₋₅ alkoxy; R³ is methyl, or methylene bound to R⁴ to form a substituted or unsubsituted cyclopropyl; R⁴ is hydrogen, or methylene bound to R³ to form the substituted or unsubsituted cyclopropyl. In certain embodiments, when R³ and R⁴ form the substituted cyclopropyl, then the cyclopropyl is substituted with difluoro. In certain embodiments, X is C—H or N. In certain embodiments, R¹⁰ is hydrogen or substituted or unsubstituted C₁₋₈alkyl. In certain embodiments, R¹⁰ is hydrogen, or methyl. In certain embodiments, X¹ is C—H; and R¹⁰ is hydrogen. In certain embodiments, X¹ is C; and R¹⁰ is methyl. In certain embodiments, X¹ is N; and R¹⁰ is hydrogen.

In one aspect, provided herein are compounds of Formula (I), and stereoisomers and pharmaceutically acceptable salt(s) thereof:

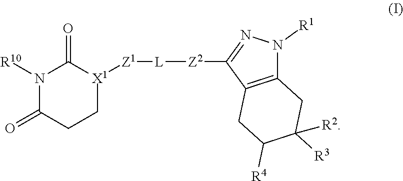

In Formula (I), R¹ is hydrogen, or methyl; R² is methyl; R³ is methyl, or methylene bound to R⁴ to form a substituted cyclopropyl; R⁴ is hydrogen, or methylene bound to R³ to form the substituted cyclopropyl. In certain embodiments, when R³ and R⁴ form the substituted cyclopropyl, then the cyclopropyl is substituted with difluoro. In certain embodiments, X¹ is C—H or N. In certain embodiments, R¹⁰ is hydrogen, or C₁₋₈alkyl. In certain embodiments, R¹⁰ is hydrogen, or methyl. In certain embodiments, X¹ is C—H; and R¹⁰ is hydrogen. In certain embodiments, X¹ is C; and R¹⁰ is methyl. In certain embodiments, X¹ is N; and R¹⁰ is hydrogen.

In certain embodiments, provided herein are compounds of Formula II, and stereoisomers and pharmaceutically acceptable salt thereof.

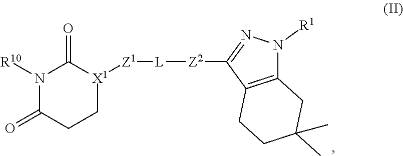

wherein the variables are described below.

In certain embodiments, provided herein are compounds of Formula (III), and stereoisomers and pharmaceutically acceptable salt thereof.

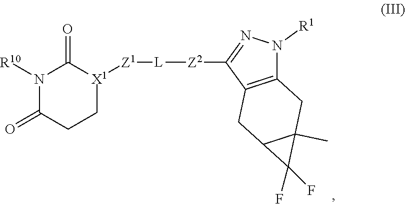

wherein the variables are described below.

In certain embodiments, provided herein are compounds of Formula (IIIa), and stereoisomers and pharmaceutically acceptable salt(s) thereof:

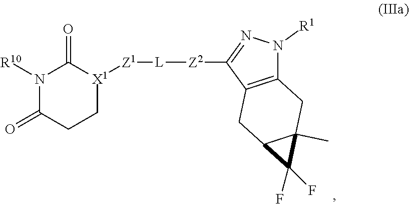

wherein the variables are described below.

In certain embodiments, provided herein are compounds of Formula (IV), and stereoisomers and pharmaceutically acceptable salt(s) thereof:

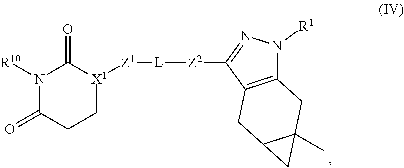

wherein the variables are described below.

In certain embodiments, provided herein are compounds of Formula (IVa), and stereoisomers and pharmaceutically acceptable salt(s) thereof:

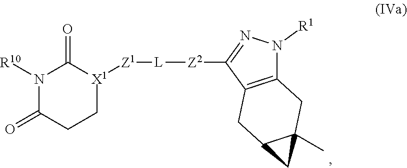

wherein the variables are described below.

In certain embodiments, provided herein are compounds of Formula (V), and stereoisomers and pharmaceutically acceptable salt thereof:

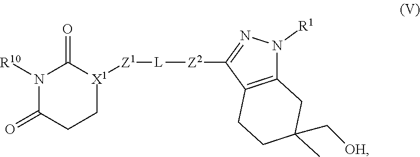

wherein the variables are described below.

In certain embodiments, provided herein are compounds of Formula (Va), and stereoisomers and pharmaceutically acceptable salt(s) thereof:

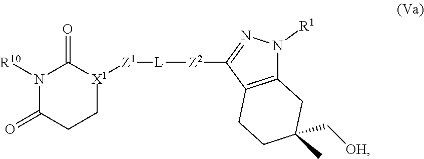

wherein the variables are described below.

In certain embodiments, provided herein are compounds of Formula (Vb), and stereoisomers and pharmaceutically acceptable salt(s) thereof:

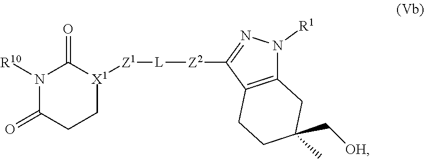

wherein the variables are described below.

In certain embodiments, provided herein are compounds of Formula (VI), and stereoisomers and pharmaceutically acceptable salt(s) thereof:

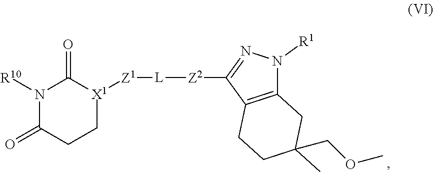

wherein the variables are described below.

In certain embodiments, provided herein are compounds of Formula (VIa), and stereoisomers and pharmaceutically acceptable salt(s) thereof:

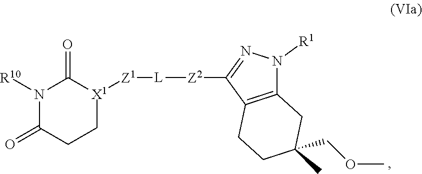

wherein the variables are described below.

In certain embodiments, provided herein are compounds of Formula (VIb), and stereoisomers and pharmaceutically acceptable salt(s) thereof:

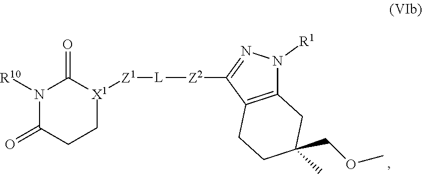

wherein the variables are described below.

In Formula (I), (II), (III), (IIIa), (IV), (IVa), (V), (Va), (Vb), (VI), (VIa), or (VIb), X¹ is C—H, or nitrogen. In Formula (I), Z¹ is a heterocycle, or —N(R′)-heterocycle. In certain embodiments, Z¹ is selected from the group consisting of

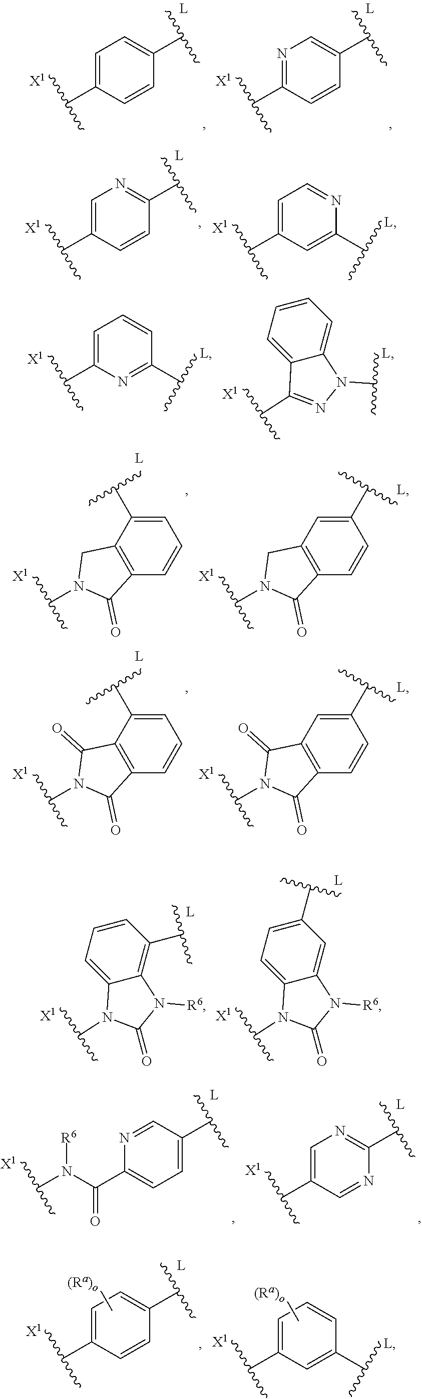

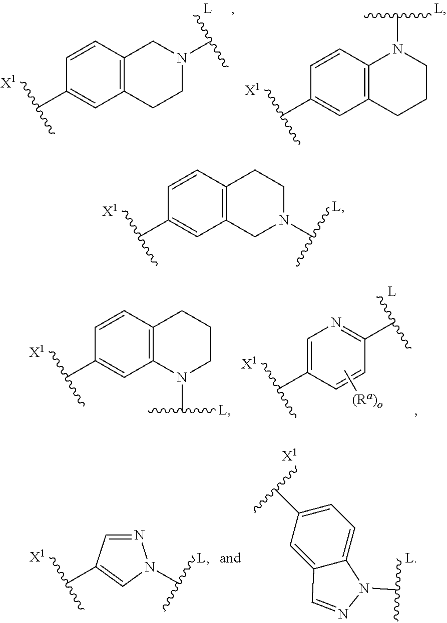

In certain embodiments, each R⁶ is hydrogen, halogen, or —C₁-C₃ alkyl. In certain embodiments, each R⁶ is hydrogen. In certain embodiments, each R⁶ is methyl. In certain embodiments, R^{a} is halogen, —C₁-C₃ alkyl, or —C₁-C₃ alkaoxy. In certain embodiments, o is 1, 2, or 3.

In the subformulae herein, the wavy lines indicate bonds to the remainder of the molecule. Unless specified otherwise, a multivalent moiety can bond to the rest of the molecule in any orientation. When orientation is specific, letters are used to indicate bonding orientation. For instance, in Z¹, the [symbol] designates attachment to X¹; and [symbol] designates attachment to L.

In Formula (I), (II), (III), (IIIa), (IV), (IVa), (V), (Va), (Vb), (VI), (VIa), or (VIb), L is a linker. The linker can be any linker suitable for linking the right and left portions of the molecule. In particular embodiments, the linker does not interfere with the harness or hook functions of the molecule. In advantageous embodiments, the linker provides useful solubility, flexibility, and/or distance between the portions of the molecule. In certain embodiments, L is a linker according to the formula -L¹-L²-L³-L⁴-L⁵-L⁶-L⁷-. Each group L^{x} is described in detail below. In certain embodiments, the linker comprises at least one heterocyclic group. In certain embodiments, the linker comprises at one heterocyclic group. In certain embodiments, the linker comprises two heterocyclic groups. In certain embodiments, the linker comprises three heterocyclic groups. In certain embodiments, the linker comprises at least one spiro bicyclic heterocycloalkylene groups. In certain embodiments, the linker comprises at one spiro bicyclic heterocycloalkylene group. In certain embodiments, the linker comprises at two spiro bicyclic heterocycloalkylene groups. In certain embodiments, the linker comprises at three spiro bicyclic heterocycloalkylene groups. In certain embodiments, the linker comprises at least one heterocycloalkylene group and at least one spiro bicyclic heterocycloalkylene. The remaining groups of the linker are selected for chemical compatibility with adjacent groups, as will be recognized by those of skill in the art.

In certain embodiments, L is a linker according to the formula -L¹-L²-L³-L⁴-L⁵-L⁶-L⁷-. In certain embodiments, -L¹- is absent, —N(R²¹)—; C(R²²)—; C₁₋₈-alkylene; C₂₋₈alkynylene; Q¹; or Q². In certain embodiments, each -L²-, -L³-, -L⁴-, and -Ls- is independently, absent; —N(R²¹)—; C(R²²)—; —C(O)—; —O—; —(CH₂—CH₂—O)₁₋₈; C₁₋₈-alkylene; C₂₋₈alkynylene; Q¹; Q²; or Q³. In certain embodiments, each -L⁶- and -L⁷-, is independently, absent; —N(R²¹)—; —C(R²²)—; —C(O)—; —C(O)—N(R²¹)—; —N(R²¹)—C(O); or —C(R²²)—C(O)—N(R²¹). In certain embodiments, L comprises at least one Q¹. In certain embodiments, L comprises one Q¹. In certain embodiments, L comprises two Q¹. In certain embodiments, L comprises three Q¹. In certain embodiments, L comprises at least one Q². In certain embodiments, L comprises one Q². In certain embodiments, L comprises two Q². In certain embodiments, L comprises three Q². In certain embodiments, L comprises at least one Q¹ and at least one Q². In certain embodiments, L comprises one Q¹ and one Q².

In certain embodiments, each Q¹ is a three- to eight-membered heterocycloalkylene comprising at least one nitrogen, wherein the heterocycloalkylene is optionally subsituted. In certain embodiments, each Q² is a five- to thirteen-membered spiro bicyclic heterocycloalkylene comprising at least one nitrogen, wherein the heterocycloalkylene is optionally subsituted. In certain embodiments, each Q³ is a three- to six-membered cycloalkylene, wherein the cycloalkylene is optionally subsituted. In certain embodiments, each R²¹ is hydrogen, or methyl. In certain embodiments, each R²² is hydrogen, methyl, aryl, or heteroaryl.

In certain embodiments of Formula (I), (II), (III), (IIIa), (IV), (IVa), (V), (Va), (Vb), (VI), (VIa), or (VIb), Z² is

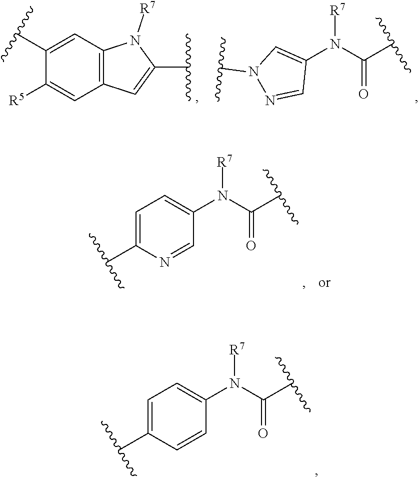

wherein R⁷ is hydrogen, halogen, or —C₁-C₃ alkyl; and R⁵ is hydrogen, halogen, or —C₁-C₃ alkyl. In certain embodiments, R⁷ is hydrogen; and R⁵ is hydrogen. In certain embodiments, R⁵ is —F. In certain embodiments, Z² is

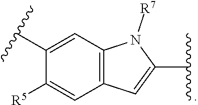

In certain embodiments, Z² is

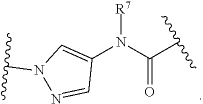

In certain embodiments, Z² is

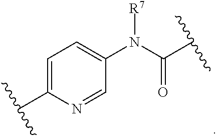

In certain embodiments, Z² is

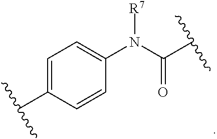

In certain embodiments of Formula (I), (II), (III), (IIIa), (IV), (IVa), (V), (Va), (Vb), (VI), (VIa), or (VIb), Z¹ is selected from the group consisting of

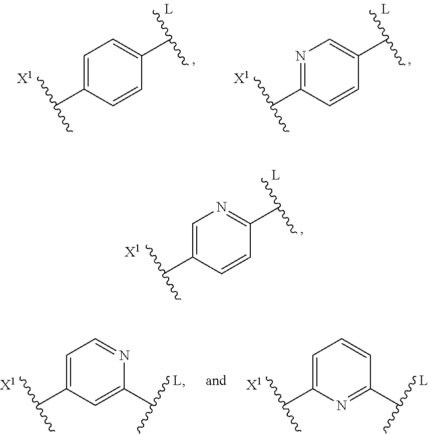

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is

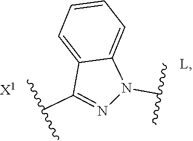

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is selected from the group consisting of

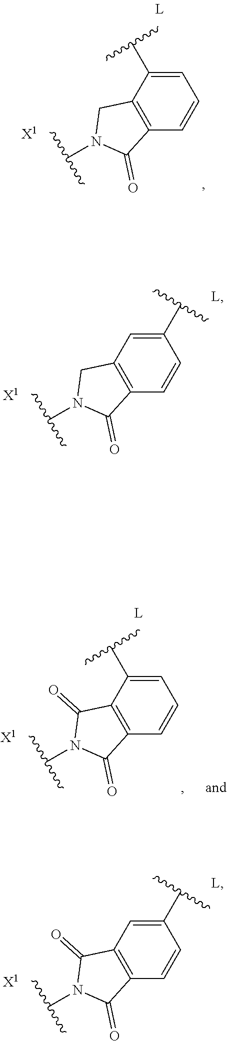

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is selected from the group consisting of

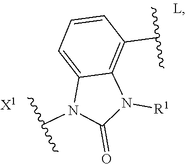

and

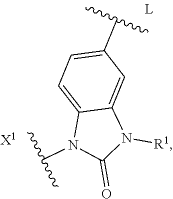

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is

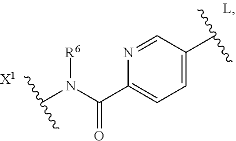

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is selected from the group consisting of

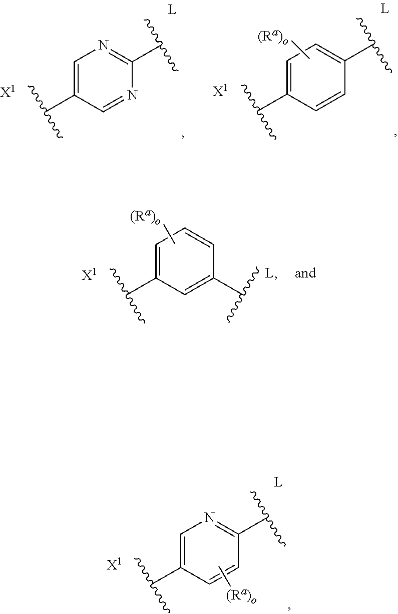

wherein [symbol] designates attachment to X¹; and [symbol] designates attachment to L. In certain embodiments, R^{a} is halogen, —C₁-C₃ alkyl, or —C₁-C₃ alkaoxy. In certain embodiments, o is 1, 2, or 3.

In certain embodiments of Formula (I), (II), (III), (IIIa), (IV), (IVa), (V), (Va), (Vb), (VI), (VIa), or (VIb), Z¹ is selected from the group consisting of

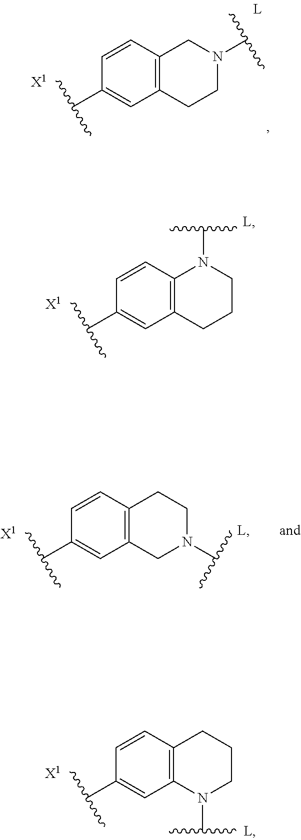

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is

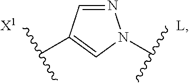

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), Z¹ is

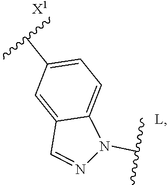

wherein [symbol] designates attachment to X¹; and [symbol] designates attachment to L.

In certain embodiments of Formula (I), (II), (III), (IIIa), (IV), (IVa), (V), (Va), (Vb), (VI), (VIa), or (VIb), L comprises at least one -Q¹- according to

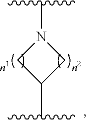

wherein n¹ is one, or two, and n² is one, or two.

In certain embodiments of Formula (I), (II), (III), (IIIa), (IV), (IVa), (V), (Va), (Vb), (VI), (VIa), or (VIb), L is selected from:
Q¹-N(Me)-CH₂-Q¹-C(O)—;
N(Me)-Q¹-CH₂-Q¹-C(O)—;
Q²-CH₂-Q¹-C(O)—;
Q¹-CH₂-Q¹-C(O)—;
Q¹-Q¹-C(O)—;
Q¹-CH₂—N(Me)-Q¹-C(O)—;
Q¹-CH₂-Q¹-CH₂—C(O)—N(Me)-;
Q¹-N(Me)-CH₂-Q¹-C(O)—;
Q¹-CH₂-Q¹-;
Q¹-CH₂-Q²-;
Q¹-CH₂—CH₂-Q¹-;
Q¹-CH₂—CH₂-Q²-;
Q¹-C(O)-Q¹-;
Q¹-C(O)-Q²-;
Q¹-CH₂-Q¹-N(Me)-C(O)—;
—CH₂—CH₂—CH₂—CH₂-Q¹-C(O)—;
Q¹-CH₂-Q³-C(O)—;
Q¹-C(O)—;
Q¹-C(O)-Q¹-C(C₆H₅)—;
C≡CCH₂-Q¹-C(O)—;
Q¹-C(O)-Q²-;
Q¹-CH₂—CH₂-Q²
Q¹-CH₂-Q¹-N—C(O)—;
—CH₂—CH₂—CH₂-Q¹-C(O)—;
Q¹-CH₂-Q¹-C(Me)-C(O)—N(Me)-;
Q¹-Q³-C(O)—;
CH₂-Q¹-;
Q¹-C(O)-Q¹-CH₂—;
—N(H)—(CH₂)₅—C(O)-Q¹-C(C₆H₅)—;
—N(H)—(CH₂)₂—O—(CH₂)₂—C(O)-Q¹-C(C₆H₅)—;
Q¹-(CH₂)₃—C(O)-Q¹-C(C₆H₅)—;
Q²-C(O)-Q¹-C(C₆H₅)—;
Q²-CH₂—C(O)-Q¹-C(C₆H₅)—;
Q²-(CH₂)₃—C(O)-Q¹-C(C₆H₅)—;
Q²-(CH₂)₂—C(O)-Q¹-C(C₆H₅)—;
(CH₂)₆-Q¹-C(C₆H₅)—;
Q¹-Q¹-C(O)-Q¹-C(C₆H₅)—;
Q¹-CH₂—C(O)-Q¹-C(C₆H₅)—;
Q¹-(CH₂)₂—C(O)-Q¹-C(C₆H₅)—;
Q¹-(CH₂)₃—C(O)-Q¹-C(C₆H₅)—;
(CH₂)₃—C(O)-Q¹-C(C₆H₅)—;
(CH₂)₄—C(O)-Q¹-C(C₆H₅)—;
(CH₂)₅—C(O)-Q¹-C(C₆H₅)—;
(CH₂)₆—C(O)-Q¹-C(C₆H₅)—;
(CH₂)₃-Q¹-CH₂—C(O)-Q¹-C(C₆H₅)—;
(CH₂)₆-Q¹-C(C₆H₅)—;
(CH₂)₆-Q¹-C(thiazolyl)-;
(CH₂)₃—O-Q³-C(O)-Q¹-C(C₆H₅)—;
(CH₂)₃—O—(CH₂)₂—C(O)-Q¹-C(C₆H₅)—;
(CH₂)₃—O—(CH₂)₂—C(O)-Q¹-C(thiazolyl)-;
(CH₂)₃—O—(CH₂)₂—C(O)-Q¹-C(pyrid-2-yl)-;
(CH₂)₄-Q¹-C(C₆H₅)—;
(CH₂)₅-Q¹-C(C₆H₅)—;
(CH₂)₆-Q¹-C(C₆H₅)—;
(CH₂)₆-Q¹-C(thiazolyl)-;
(CH₂)₆-Q¹-C(pyrid-2-yl)-;
(CH₂)₇-Q¹-C(C₆H₅)—;
(CH₂)₇-Q¹-C(Me)-C(O)—N(Me)-;
—N(H)—(CH₂)₂—O—(CH₂)₂-Q¹-C(Me)-C(O)—N(Me)-;
(CH₂)₃—O—(CH₂)₂—C(O)-Q¹-C(Me)-C(O)—N(Me)-;
—N(H)—(CH₂)₂—O—(CH₂)₂-Q¹-C(C₆H₅)—;
—N(H)—(CH₂)₂—O—(CH₂)₂—C(O)-Q¹-C(C₆H₅)—;
—N(H)—(CH₂)₂—[O—(CH₂)_{2]2}—C(O)-Q¹-C(C₆H₅)—;
—N(H)—(CH₂)₂—[O—(CH₂)_{2]3}—C(O)-Q¹-C(C₆H₅)—;
—N(H)—(CH₂)₂—[O—(CH₂)_{2]4}—C(O)-Q¹-C(C₆H₅)—;
—N(H)—(CH₂)₂—[O—(CH₂)_{2]5}—C(O)-Q¹-C(C₆H₅)—;
—N(H)—(CH₂)₂—[O—(CH₂)_{2]6}—C(O)-Q¹-C(C₆H₅)—;
—N(H)—(CH₂)₂—[O—(CH₂)_{2]7}—C(O)-Q¹-C(C₆H₅)—;
—N(H)—(CH₂)₂—[O—(CH₂)₂]s-C(O)-Q¹-C(C₆H₅)—;
—N(H)-Q³-O—(CH₂)₂—CH₂—;
—N(H)—(CH₂)₃-Q¹-(CH₂)₂—;
—C(O)—N(H)—[(CH₂)₃—O]₃—(CH₂)₂—NH—;
—C(O)—N(H)—[(CH₂)₃-O]₃-(CH₂)₂—;
Q¹-C(O)—[(CH₂)₂—O]₃—(CH₂)₂—NH—;
Q¹-(CH₂)₃—O—CH₂—;
Q¹-C(O)—(C₆H₆)—CH₂—;
Q¹-(2-pyridyl)-O—CH₂—;
—N(H)-Q³-X-(2-pyridyl)-O—;
—N(H)-Q³-X-(4-pyridyl)-;
—N(H)—(CH₂)₂-Q³-X-(2-pyridyl)-O—CH₂—;
CH≡C—(CH₂)₂Q¹-; a
Q¹-;
Q¹-CH₂—C(O)—N(CH₃)—;
Q¹-CH₂-Q¹-C(O)—;
Q¹-CH₂—CH₂-Q¹-C(O)—;
Q¹-N(CH₃)—C(O)-Q¹-C(O)—;
CH₂-Q¹-C(O)—;
Q¹-CH₂-Q¹-;
Q(-H—C(O)-Q¹-;
O-Q¹-C(O)-Q¹-;
—C(O)-Q¹-;
Q¹-C(O)-Q¹-;
CH₂—C(O)-Q¹-;
—C(O)-Q¹-CH₂-Q¹-;
—C(O)-Q¹-CH₂-Q¹-C(O)—;
CH₂-Q¹-CH₂-Q¹-C(O)—;
—C(O)-Q¹-CH₂-Q¹-CH₂—;
Q¹-CH₂-Q¹-CH₂—;
Q¹-C(O)-Q¹-CH₂—; and
—CH₂-Q¹-. In some embodiments, X is oxygen or sulfur.

In certain embodiments, L comprises at least one -Q¹- selected from the group consisting of

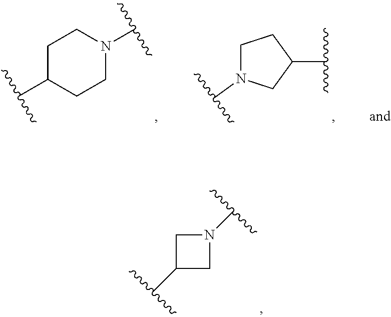 [SMILES: *C1CCN(*)CC1 ; *C1CCN(*)C1 ; *C1CN(*)C1]

wherein -Q¹- is optionally mono- or di-substituted with a group independently selected from —F, —CH₃, and —CH₂CH₃.

In certain embodiments, L comprises at least one -Q²- according to

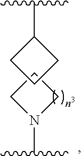

wherein n³ is one or two.

In certain embodiments, L comprises at least one -Q²- according to

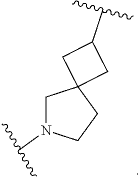 [SMILES: *C1CC2(CCN(*)C2)C1]

In certain embodiments, L comprises at least one -Q²- according to

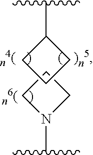

wherein n⁴ is one, or two, n⁵ is one, or two, and n⁶ is one, or two.

In certain embodiments, L comprises at least one -Q²- according to

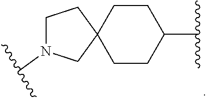 [SMILES: *C1CCC2(CC1)CCN(*)C2]

In certain embodiments, L comprises at least one -Q²- according to

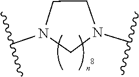

wherein n⁸ is one, or two, wherein -Q²- is optionally mono- or di-substituted with a group independently selected from —F, —CH₃, and —CH₂CH₃.

In certain embodiments, L comprises at least one -Q²- according to

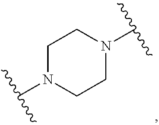 [SMILES: *N1CCN(*)CC1]

wherein -Q²- is optionally mono- or di-substituted with a group independently selected from —F, —CH₃, and —CH₂CH₃.

In certain embodiments, L comprises at least one -Q²- according to

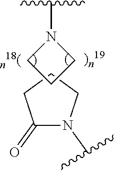

wherein n¹⁸ and n¹⁹ is two, or piperidinyl; or when n¹⁸ is two, then n¹⁹ is three, or azepanyl; or when n¹⁸ is three, then n¹⁹ is two, or azepanyl.

In certain embodiments, L comprises at least one -Q²- according to

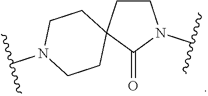 [SMILES: *N1CCC2(CC1)CCN(*)C2=O]

In certain embodiments, L comprises at least one -Q²- according to

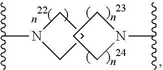

wherein n²² is zero to two; n²³ is zero to two, and n²⁴ is one, or two. In certain embodiments, when n²² is two, then n²³ and n²⁴ is one; or when n²² is two, then n²³ and n²⁴ is two.

In certain embodiments, L comprises at least one -Q²- according to

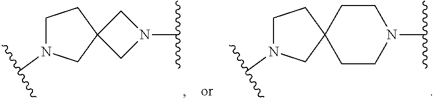 [SMILES: *N1CCC2(C1)CN(*)C2 ; *N1CCC2(CC1)CCN(*)C2]

In certain embodiments, L comprises at least one -Q²- according to

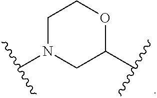 [SMILES: *C1CN(*)CCO1]

In certain embodiments, L comprises at least one -Q³- according to

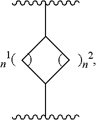

wherein n¹ is one, or two, and n² is one, or two.

In certain embodiments, L comprises at least one -Q³- selected from the group consisting of

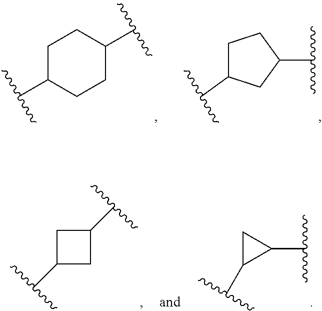 [SMILES: *C1CCC(*)CC1 ; *C1CCC(*)C1 ; *C1CC(*)C1 ; *C1CC1*]

In certain embodiments, the linker group L is —C(O)—.

In certain embodiments, of any compound of Formulas (I), (II), (III), (IIIa), (IV), (IVa), (V), (Va), (Vb), (VI), (VIa), or (VIb), the linker group L is selected from:

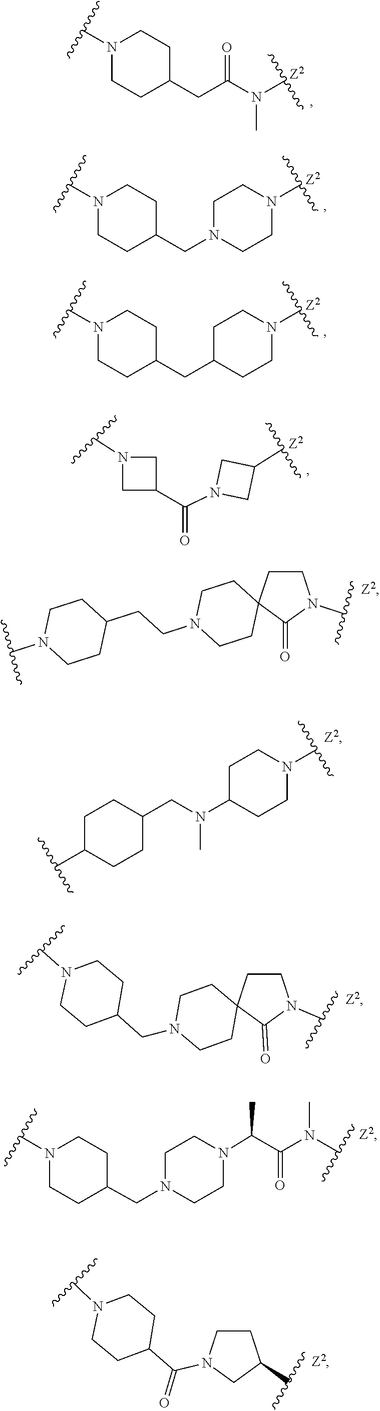

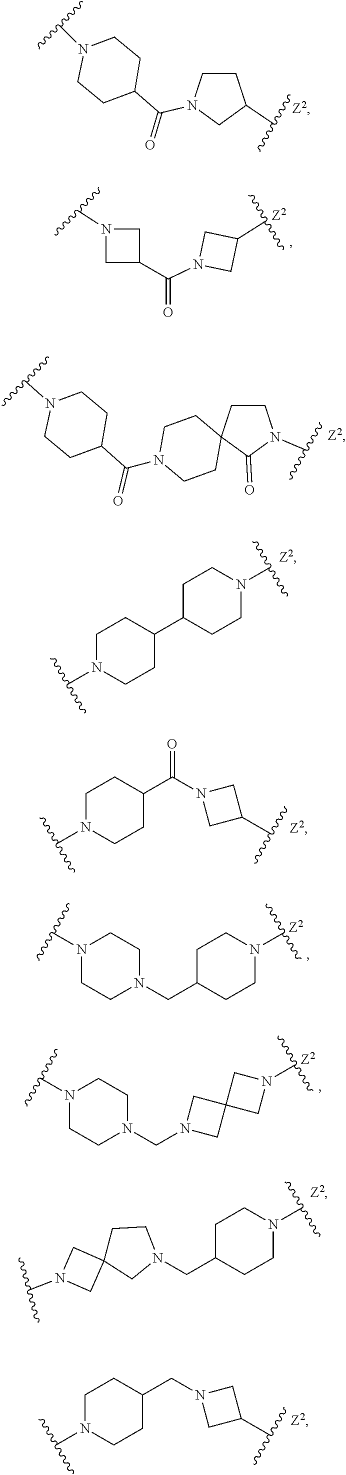

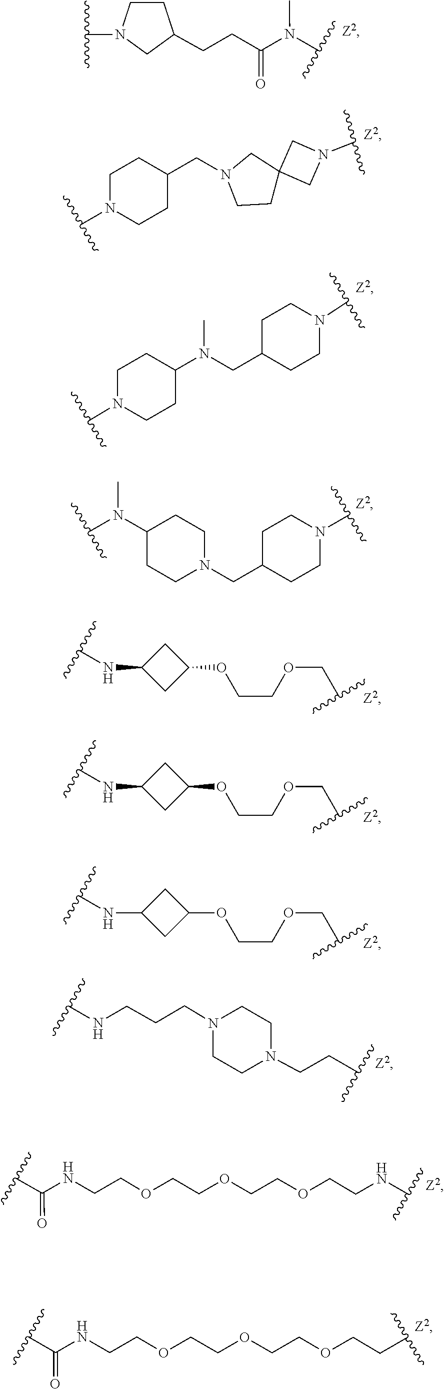

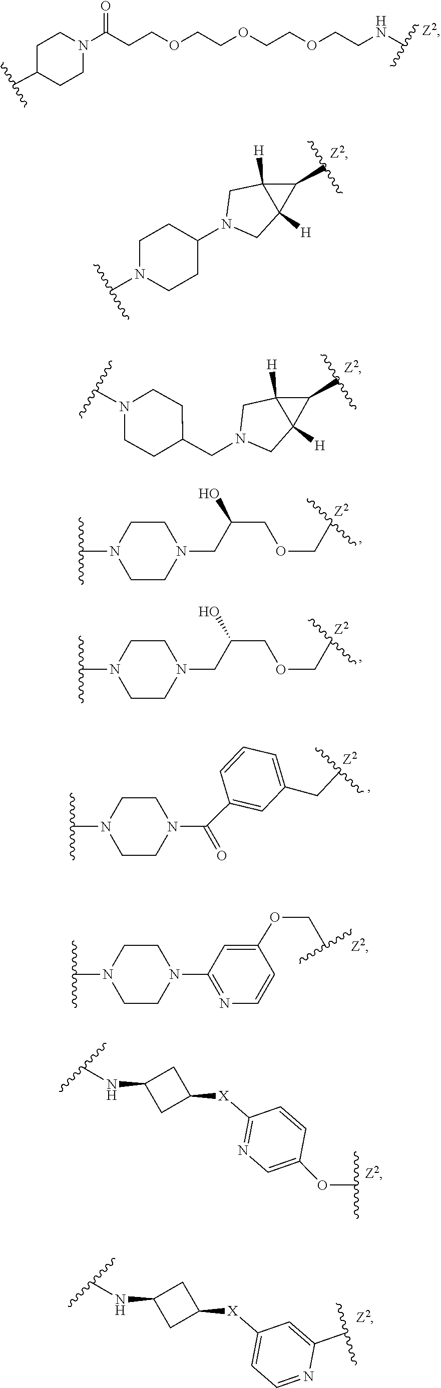

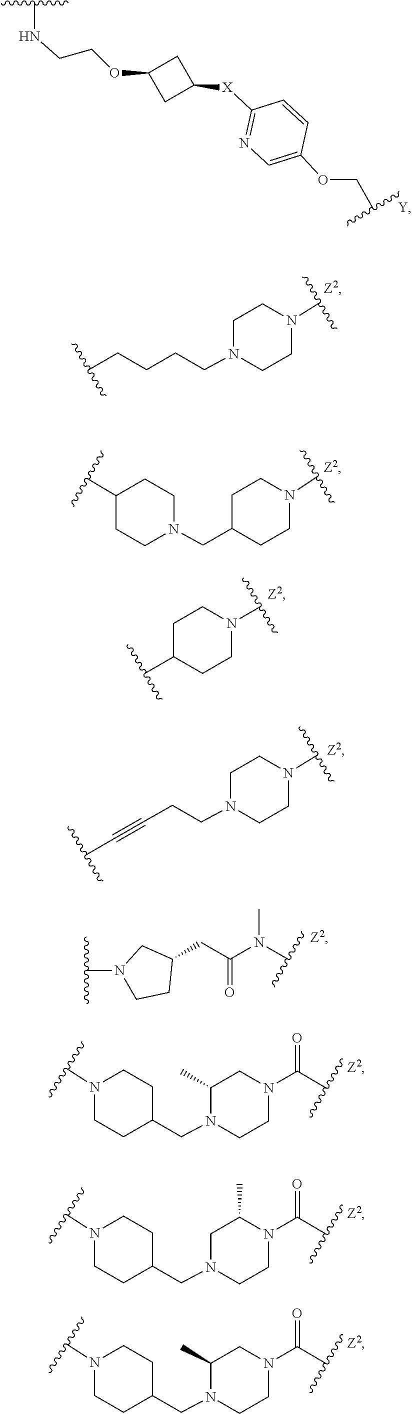

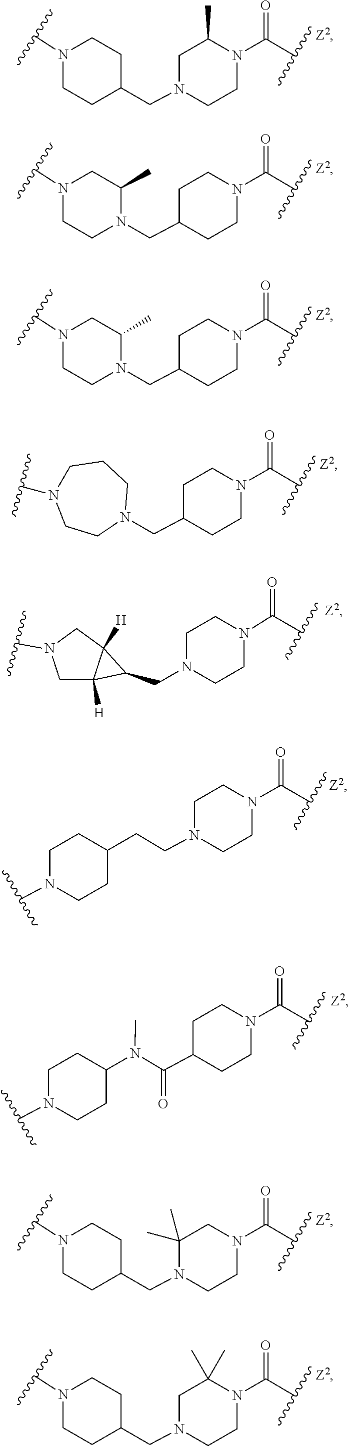

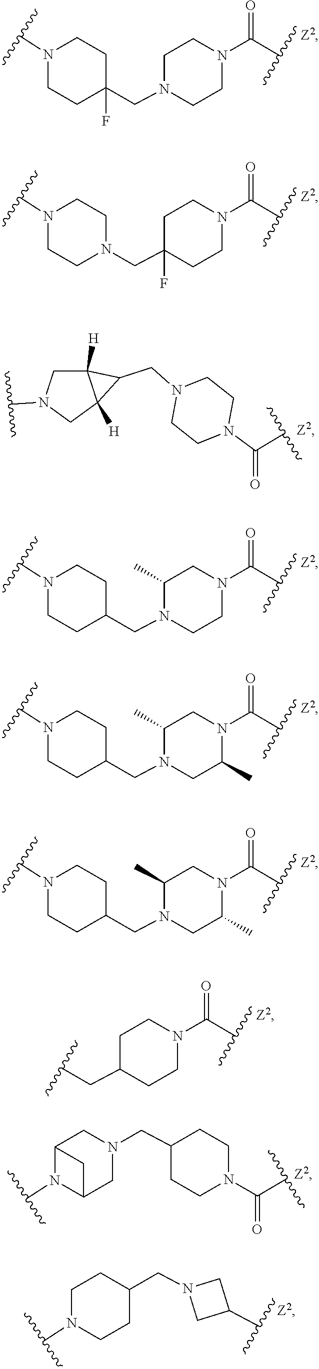

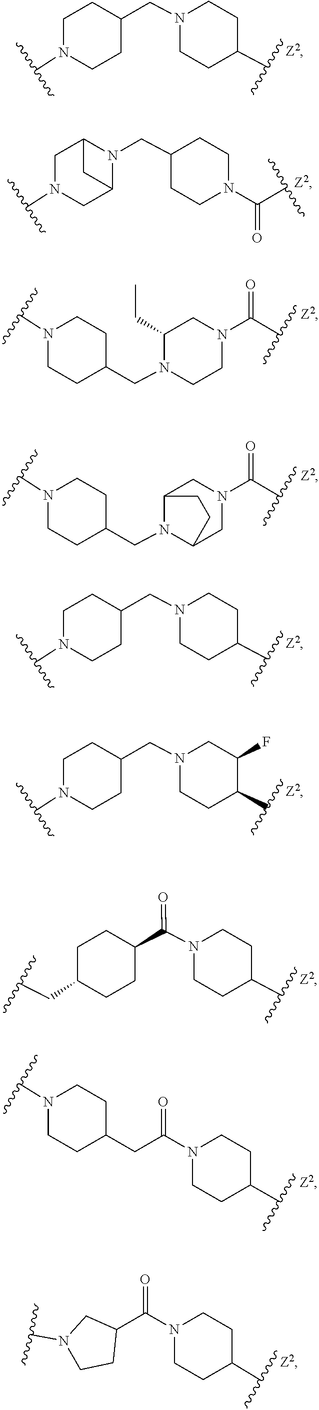

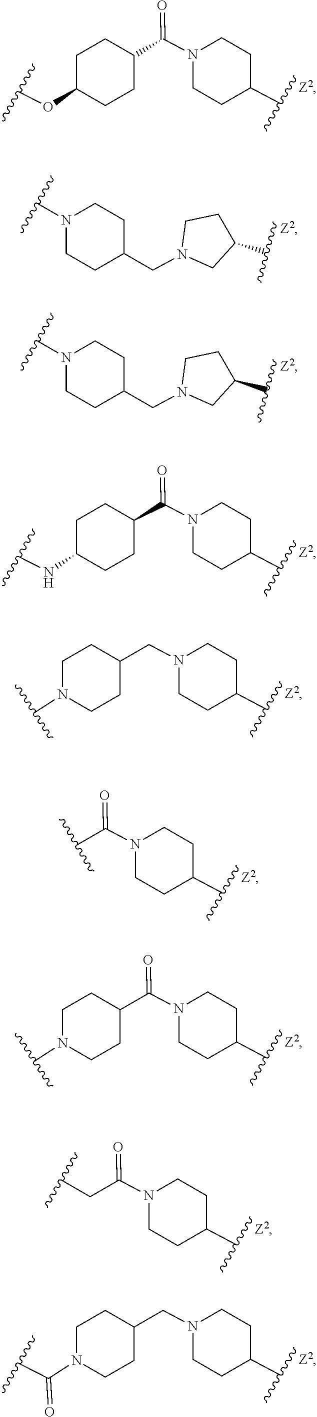

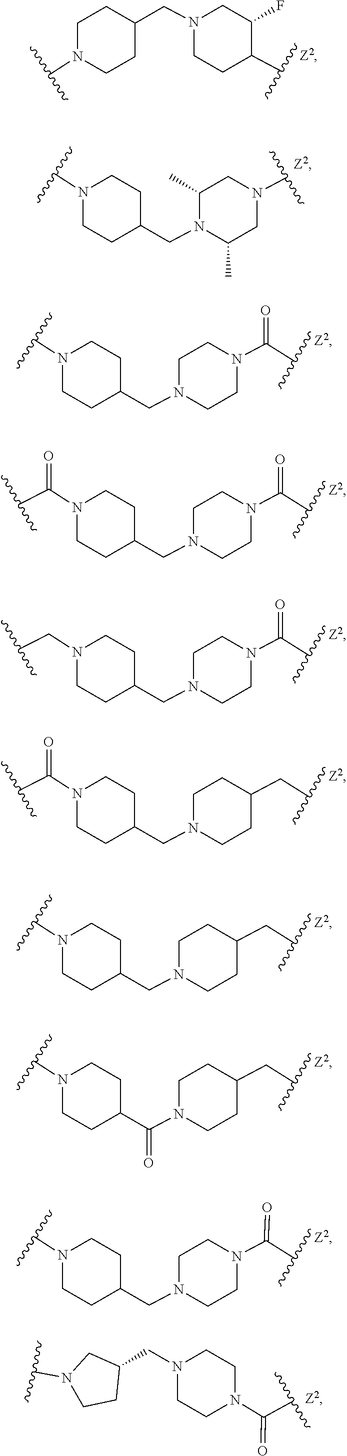

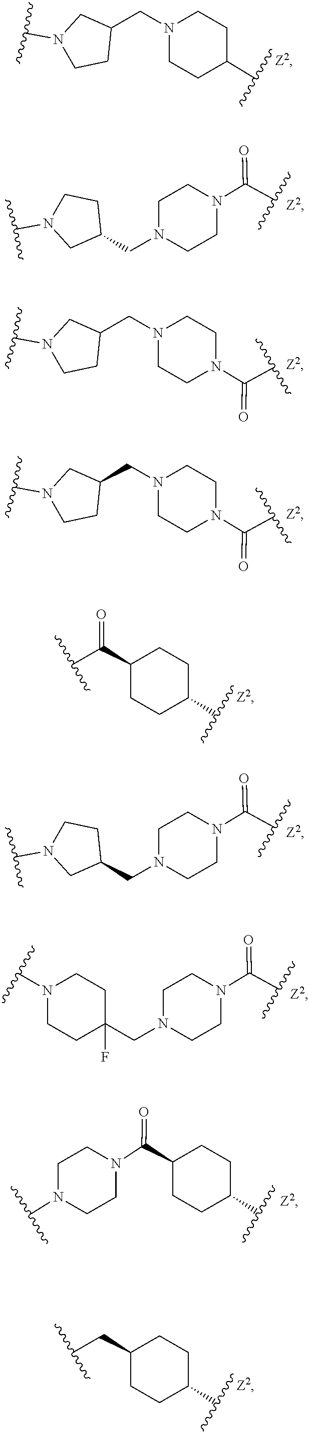

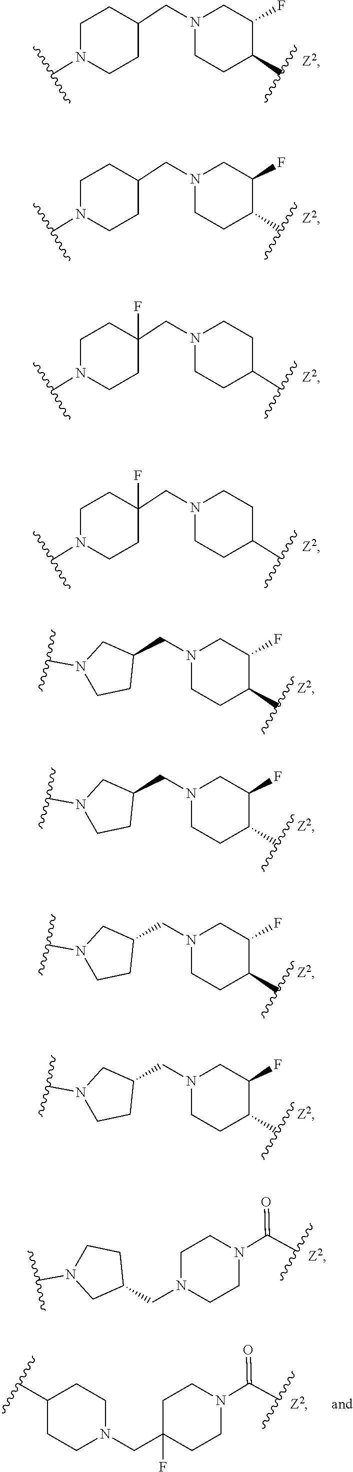

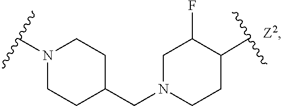

wherein, [symbol] designates attachment to Z; wherein X is oxygen or sulfur.

In certain embodiments of Formula (I), (II), (III), or (IIIa), Z¹ is

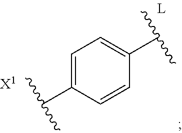

### and Z² is

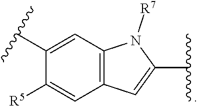

In certain embodiments of Formula (I), (II), (III), or (IIa), Z¹ is

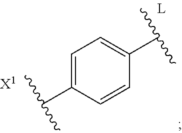

### and Z² is

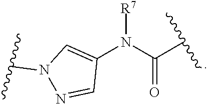

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R is hydrogen; R¹⁰ is methyl; X¹ is N; R⁵ is hydrogen; and R⁷ is methyl. In certain embodiments of this paragraph, R is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

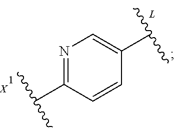

### and Z² is

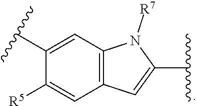

In certain embodiments of Formula (I), (II), (III), or (IIIa), Z¹ is

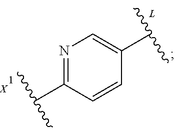

### and Z² is

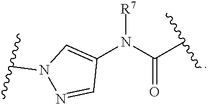

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

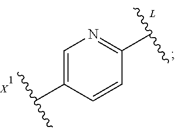

### and Z² is

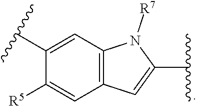

In certain embodiments of Formula (I), (II), (III), or (IIIa), Z¹ is

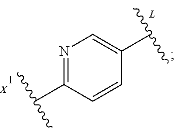

### and Z² is

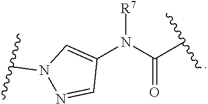

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

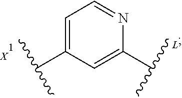

### and Z² is

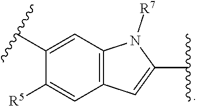

In certain embodiments of Formula (I), (II), (III), or (IIIa), Z¹ is

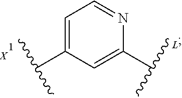

### and Z² is

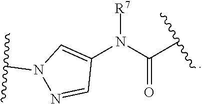

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

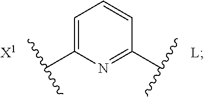

### and Z² is

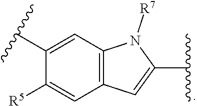

In certain embodiments of Formula (I), (II), (III), or (IIIa), Z¹ is

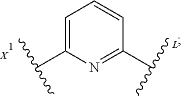

### and Z² is

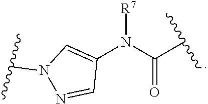

In certain embodiments of this paragraph, R¹ is hydrogen; R¹⁰ is hydrogen; X is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R is hydrogen; R¹⁰ is methyl; X¹ is N; R⁵ is hydrogen; and R⁷ is methyl. In certain embodiments of this paragraph, R is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

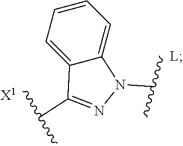

### and Z² is

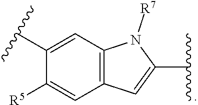

In certain embodiments of Formula (I), (II), (III), or (IIIa), Z¹ is

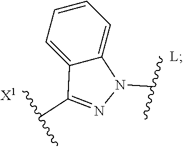

### and Z² is

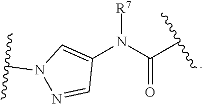

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

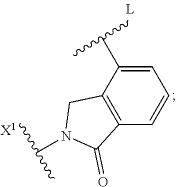

### and Z² is

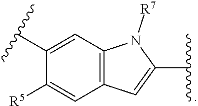

In certain embodiments of Formula (I), (II), (III), or (IIIa), Z¹ is

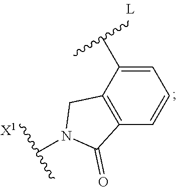

### and Z² is

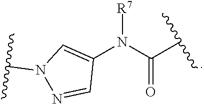

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, 1 is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

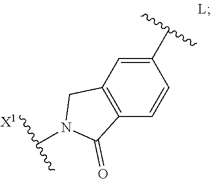

### and Z² is

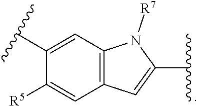

In certain embodiments of Formula (I), (II), (III), or (IIIa), Z¹ is

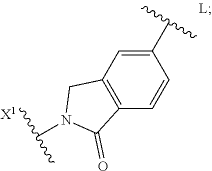

### and Z² is

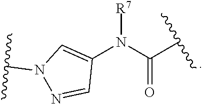

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

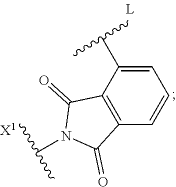

### and Z² is

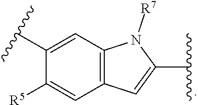

In certain embodiments of Formula (I), (II), (III), or (IIIa), Z¹ is

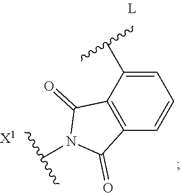

### and Z² is

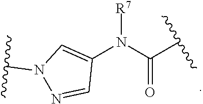

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R¹ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

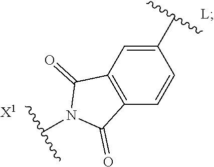

### and Z² is

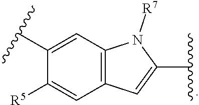

In certain embodiments of Formula (I), (II), (III), or (IIIa), Z¹ is

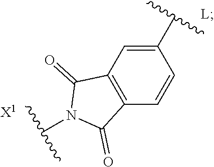

### and Z² is

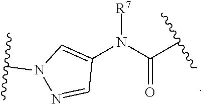

In certain embodiments of this paragraph, R¹ is hydrogen; R¹⁰ is hydrogen; X¹ is C—H; R¹ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R¹ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), or (IIIa), Z¹ is

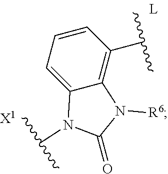

### and Z² is

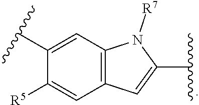

In certain embodiments of Formula (I), (II), (III), or (IIIa), Z¹ is

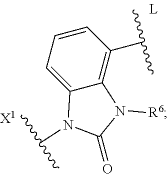

### and Z² is

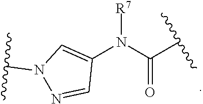

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), or (IIIa), Z¹ is

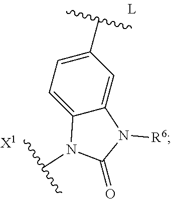

### and Z² is

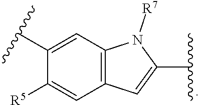

In certain embodiments of Formula (I), (II), (III), or (IIIa), Z¹ is

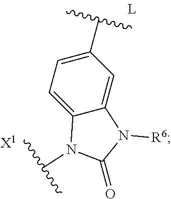

### and Z² is

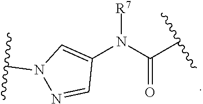

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R is hydrogen; R¹⁰ is methyl; X¹ is N; R⁵ is hydrogen; and R⁷ is methyl. In certain embodiments of this paragraph, R is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), or (IIIa), Z¹ is

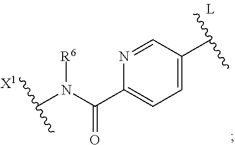

### and Z² is

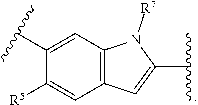

In certain embodiments of Formula (I), (II), (III), or (IIIa), Z¹ is

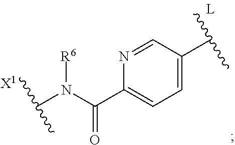

### and Z² is

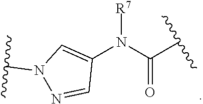

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

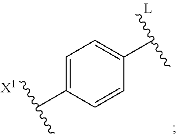

### and Z² is

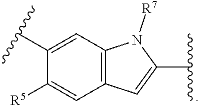

In certain embodiments of Formula (I), (II), (III), (IIIa), (IV), (IVa), (V), (Va), (Vb), (VI), (VIa), or (VIb), Z¹ is

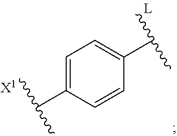

### and Z² is

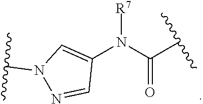

In certain embodiments of Formula (I), (II), (III), (IIIa), (IV), (IVa), (V), (Va), (Vb), (VI), (VIa), or (VIb), Z¹ is

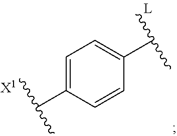

### and Z² is

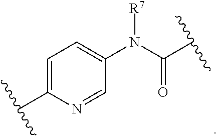

In certain embodiments of Formula (I), (II), (III), (IIIa), (IV), (IVa), (V), (Va), (Vb), (VI), (VIa), or (VIb), Z¹ is

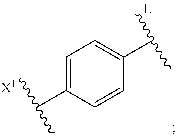

### and Z² is

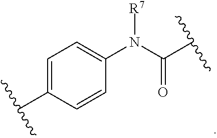

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is C—H; R⁵ is —F; and R⁷ is hydrogen. In certain embodiments of this paragraph, R¹ is hydrogen; R¹⁰ is hydrogen; X¹ is C—H; R⁵ is —F; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is —F; and R⁷ is hydrogen. In certain embodiments of this paragraph, R¹ is hydrogen; R¹⁰ is hydrogen; X¹ is N; R⁵ is —F; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

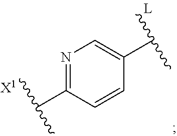

### and Z² is

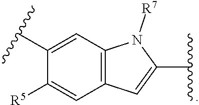

In certain embodiments of Formula (I), (II), (III), (IIIa), (IV), (IVa), (V), (Va), (Vb), (VI), (VIa), or (VIb), Z¹ is

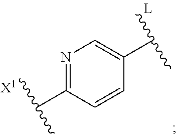

### and Z² is

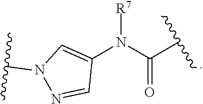

In certain embodiments of Formula (I), (II), (III) (IIIa), (IV), (IVa), (V), (Va), (Vb), (VI), (VIa), or (VIb), Z¹ is

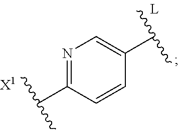

### and Z² is

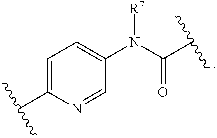

In certain embodiments of Formula (I), (II), (III), (IIIa), (IV), (IVa), (V), (Va), (Vb), (VI), (VIa), or (VIb), Z¹ is

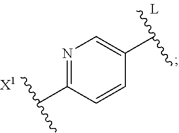

### and Z² is

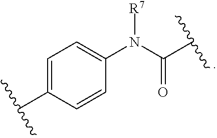

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; Rt is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

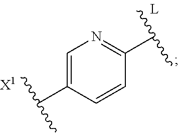

### and Z² is

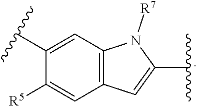

In certain embodiments of Formula (I), (II), (III), (IIIa), (IV), (IVa), (V), (Va), (Vb), (VI), (VIa), or (VIb), Z¹ is

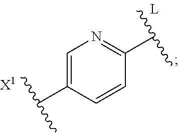

### and Z² is

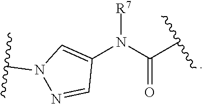

In certain embodiments of Formula (I), (II), (III), (IIIa), (IV), (IVa), (V), (Va), (Vb), (VI), (VIa), or (VIb), Z¹ is

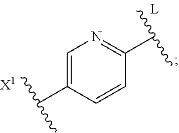

### and Z² is

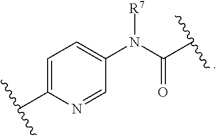

In certain embodiments of Formula (I), (II), (III), (IIIa), (IV), (IVa), (V), (Va), (Vb), (VI), (VIa), or (VIb), Z¹ is

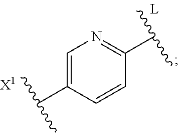

### and Z² is

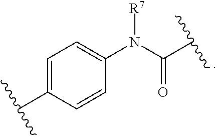

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

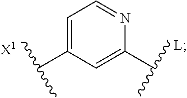

### and Z² is

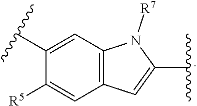

In certain embodiments of Formula (I), (II), (III), (IIIa), (IV), (IVa), (V), (Va), (Vb), (VI), (VIa), or (VIb), Z¹ is

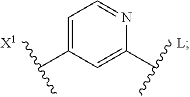

### and Z² is

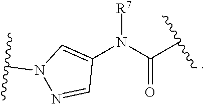

In certain embodiments of Formula (I), (II), (III), (IIIa), (IV), (IVa), (V), (Va), (Vb), (VI), (VIa), or (VIb), Z¹ is

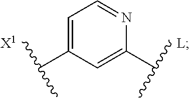

### and Z² is

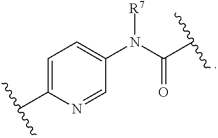

In certain embodiments of Formula (I), (II), (III), (IIIa), (IV), (IVa), (V), (Va), (Vb), (VI), (VIa), or (VIb), Z¹ is

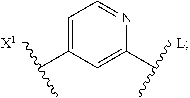

### and Z² is

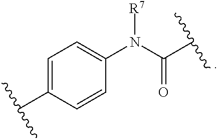

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

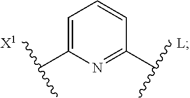

### and Z² is

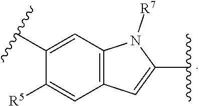

In certain embodiments of Formula (I), (II), (III), (IIIa), (IV), (IVa), (V), (Va), (Vb), (VI), (VIa), or (VIb), Z¹ is

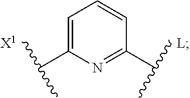

### and Z² is

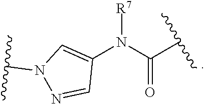

In certain embodiments of Formula (I), (II), (III), (IIIa), (IV), (IVa), (V), (Va), (Vb), (VI), (VIa), or (VIb), Z¹ is

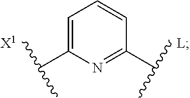

### and Z² is

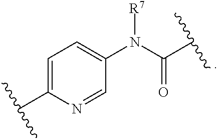

In certain embodiments of Formula (I), (II), (III), (IIIa), (IV), (IVa), (V), (Va), (Vb), (VI), (VIa), or (VIb), Z¹ is

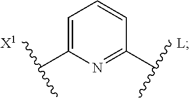

### and Z² is

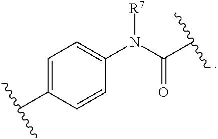

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

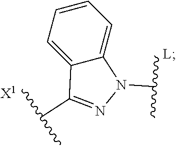

### and Z² is

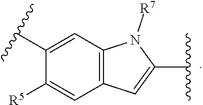

In certain embodiments of Formula (I), (II), (III), (IIIa), (IV), (IVa), (V), (Va), (Vb), (VI), (VIa), or (VIb), Z¹ is

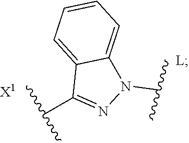

### and Z² is

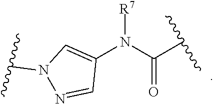

In certain embodiments of Formula (I), (II), (III), (IIIa), (IV), (IVa), (V), (Va), (Vb), (VI), (VIa), or (VIb), Z¹ is

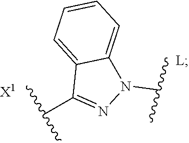

### and Z² is

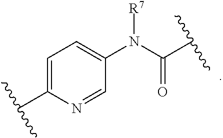

In certain embodiments of Formula (I), (II), (III), (IIIa), (IV), (IVa), (V), (Va), (Vb), (VI), (VIa), or (VIb), Z¹ is

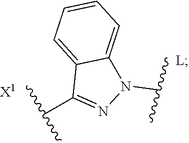

### and Z² is

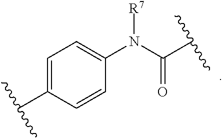

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, 1 is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

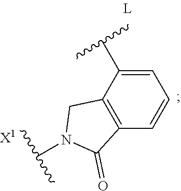

### and Z² is

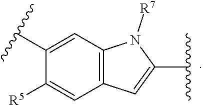

In certain embodiments of Formula (I), (II), (III), (IIIa), (IV), (IVa), (V), (Va), (Vb), (VI), (VIa), or (VIb), Z¹ is

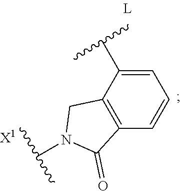

### and Z² is

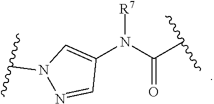

In certain embodiments of Formula (I), (II), (III), (IIIa), (IV), (IVa), (V), (Va), (Vb), (VI), (VIa), or (VIb), Z¹ is

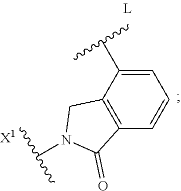

### and Z² is

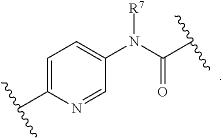

In certain embodiments of Formula (I), (II), (III), (IIIa), (IV), (IVa), (V), (Va), (Vb), (VI), (VIa), or (VIb), Z¹ is

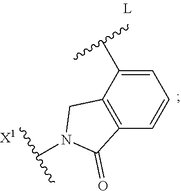

### and Z² is

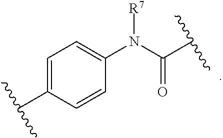

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

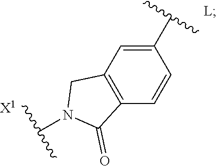

### and Z² is

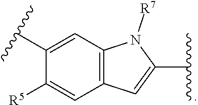

In certain embodiments of Formula (I), (II), (III), (IIIa), (IV), (IVa), (V), (Va), (Vb), (VI), (VIa), or (VIb), Z¹ is

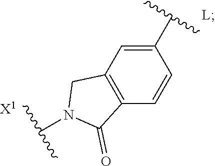

### and Z² is

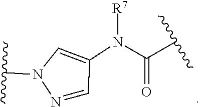

In certain embodiments of Formula (I), (II), (III), (IIIa), (IV), (IVa), (V), (Va), (Vb), (VI), (VIa), or (VIb), Z¹ is

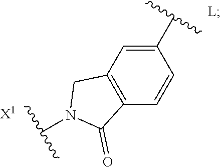

### and Z² is

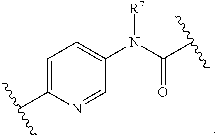

In certain embodiments of Formula (I), (II), (III), (IIIa), (IV), (IVa), (V), (Va), (Vb), (VI), (VIa), or (VIb), Z¹ is

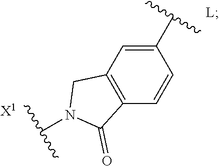

### and Z² is

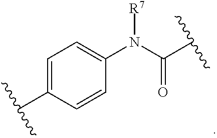

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

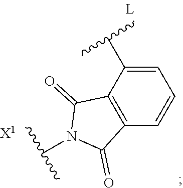

### and Z² is

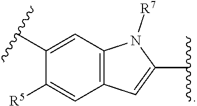

In certain embodiments of Formula (I), (II), (III), (IIIa), (IV), (IVa), (V), (Va), (Vb), (VI), (VIa), or (VIb), Z¹ is

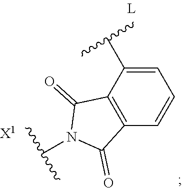

### and Z² is

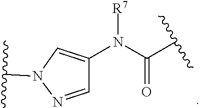

In certain embodiments of Formula (I), (II), (III), (IIIa), (IV), (IVa), (V), (Va), (Vb), (VI), (VIa), or (VIb), Z¹ is

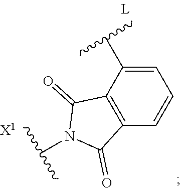

### and Z² is

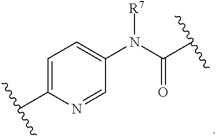

In certain embodiments of Formula (I), (II), (III), (IIIa), (IV), (IVa), (V), (Va), (Vb), (VI), (VIa), or (VIb), Z¹ is

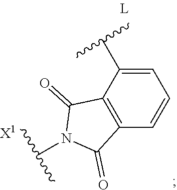

### and Z² is

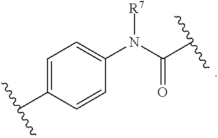

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

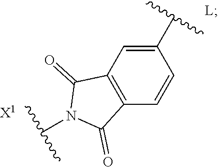

### and Z² is

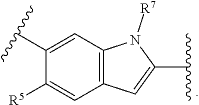

In certain embodiments of Formula (I), (II), (III), (IIIa), (IV), (IVa), (V), (Va), (Vb), (VI), (VIa), or (VIb), Z¹ is

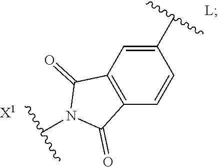

### and Z² is

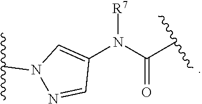

In certain embodiments of Formula (I), (II), (III), (IIIa), (IV), (IVa), (V), (Va), (Vb), (VI), (VIa), or (VIb), Z¹ is

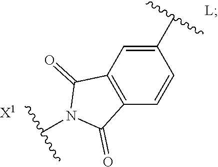

### and Z² is

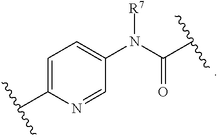

In certain embodiments of Formula (I), (II), (III), (IIIa), (IV), (IVa), (V), (Va), (Vb), (VI), (VIa), or (VIb), Z¹ is

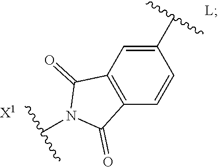

### and Z² is

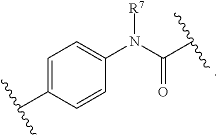

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl.

In certain embodiments of Formula (I), (II), (III), (IIIa), (IV), (IVa), (V), (Va), (Vb), (VI), (VIa), or (VIb), Z¹ is

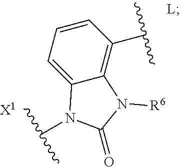

### and Z² is

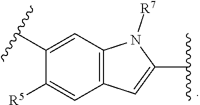

In certain embodiments of Formula (I), (II), (III), (IIIa), (IV), (IVa), (V), (Va), (Vb), (VI), (VIa), or (VIb), Z¹ is

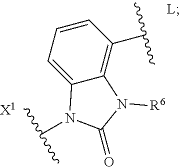

### and Z² is

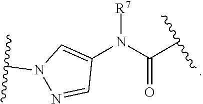

In certain embodiments of Formula (I), (II), (III), (IIIa), (IV), (IVa), (V), (Va), (Vb), (VI), (VIa), or (VIb), Z¹ is

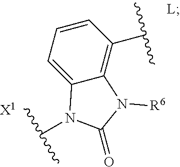

### and Z² is

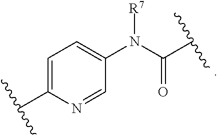

In certain embodiments of Formula (I), (II), (III), (IIIa), (IV), (IVa), (V), (Va), (Vb), (VI), (VIa), or (VIb), Z¹ is

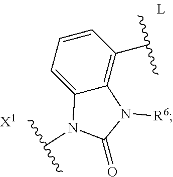

### and Z² is

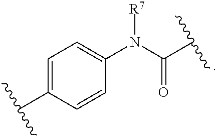

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

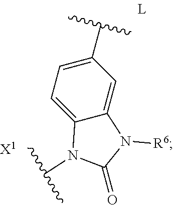

### and Z² is

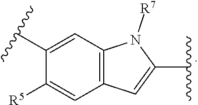

In certain embodiments of Formula (I), (II), (III), (IIIa), (IV), (IVa), (V), (Va), (Vb), (VI), (VIa), or (VIb), Z¹ is

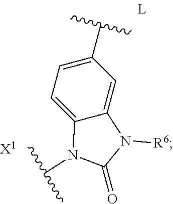

### and Z² is

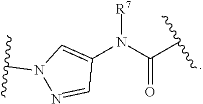

In certain embodiments of Formula (I), (II), (III), (IIIa), (IV), (IVa), (V), (Va), (Vb), (VI), (VIa), or (VIb), Z¹ is

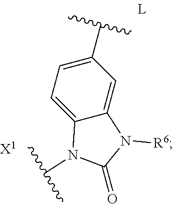

### and Z² is

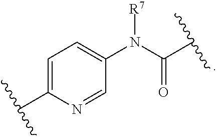

In certain embodiments of Formula (I), (II), (III), (IIIa), (IV), (IVa), (V), (Va), (Vb), (VI), (VIa), or (VIb), Z¹ is

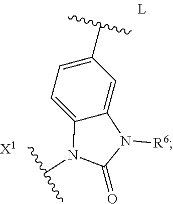

### and Z² is

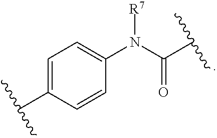

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

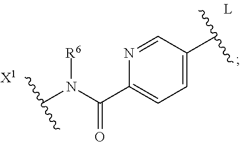

### and Z² is

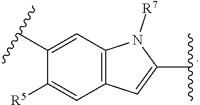

In certain embodiments of Formula (I), (II), (III), (IIIa), (IV), (IVa), (V), (Va), (Vb), (VI), (VIa), or (VIb), Z¹ is

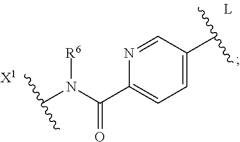

### and Z² is

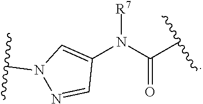

In certain embodiments of Formula (I), (II), (III), (IIIa), (IV), (IVa), (V), (Va), (Vb), (VI), (VIa), or (VIb), Z¹ is

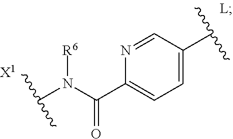

### and Z² is

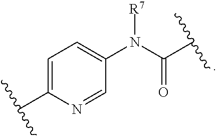

In certain embodiments of Formula (I), (II), (III), (IIIa), (IV), (IVa), (V), (Va), (Vb), (VI), (VIa), or (VIb), Z¹ is

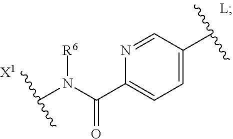

### and Z² is

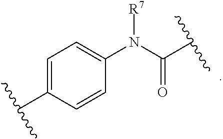

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R is halogen; and R⁷ is hydrogen. In certain embodiments of this paragraph, R¹ is hydrogen; R¹⁰ is methyl; X¹ is C—H; R¹ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R is hydrogen; and R⁷ is hydrogen. In certain embodiments of this paragraph, R¹ is methyl; R¹⁰ is hydrogen; X¹ is C—H; R¹ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R¹ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R¹ is halogen; and R⁷ is methyl. In certain embodiments of this paragraph, R¹ is methyl; R¹⁰ is methyl; X¹ is C—H; TI is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R¹ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

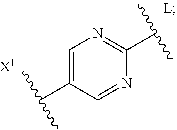 [SMILES: "X1" ; *c1cnc(*)nc1]

### and Z² is

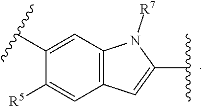

In certain embodiments of Formula (I), (II), (III), (IIIa), (IV), (IVa), (V), (Va), (Vb), (VI), (VIa), or (VIb), Z¹ is

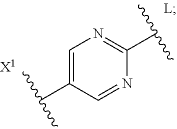 [SMILES: "X1" ; *c1cnc(*)nc1]

### and Z² is

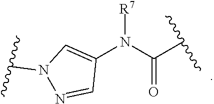

In certain embodiments of Formula (I), (II), (III), (IIIa), (IV), (IVa), (V), (Va), (Vb), (VI), (VIa), or (VIb), Z¹ is

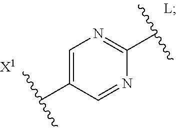 [SMILES: "X1" ; *c1cnc(*)nc1]

### and Z² is

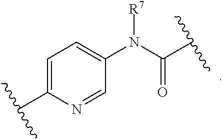

In certain embodiments of Formula (I), (II), (III), (IIIa), (IV), (IVa), (V), (Va), (Vb), (VI), (VIa), or (VIb), Z¹ is

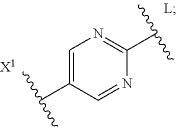 [SMILES: "X1" ; *c1cnc(*)nc1]

### and Z² is

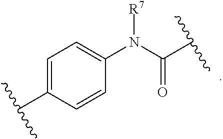

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

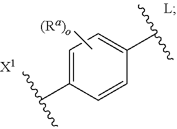

### and Z² is

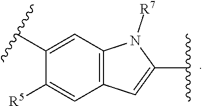

In certain embodiments of Formula (I), (II), (III), (IIIa), (IV), (IVa), (V), (Va), (Vb), (VI), (VIa), or (VIb), Z¹ is

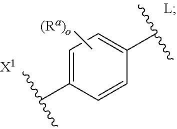

### and Z² is

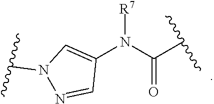

In certain embodiments of Formula (I), (II), (III), (IIIa), (IV), (IVa), (V), (Va), (Vb), (VI), (VIa), or (VIb), Z¹ is

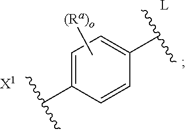

### and Z² is

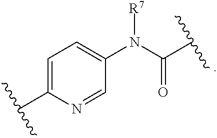

In certain embodiments of Formula (I), (II), (III), (IIIa), (IV), (IVa), (V), (Va), (Vb), (VI), (VIa), or (VIb), Z¹ is

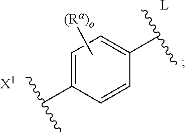

### and Z² is

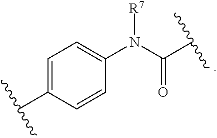

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl. In certain embodiments of this paragraph, R^{a} is halogen, —C₁-C₃ alkyl, or —C₁-C₃ alkaoxy. In certain embodiments of this paragraph, R^{a} is halogen. In certain embodiments of this paragraph, R^{a} is —C₁-C₃ alkyl. In certain embodiments of this paragraph, R^{a} is —C₁-C₃ alkaoxy. In certain embodiments of this paragraph, o is 1, 2, 3, or 4. In certain embodiments of this paragraph, o is 1. In certain embodiments of this paragraph, o is 2. In certain embodiments of this paragraph, o is 3. In certain embodiments of this paragraph, o is 4.

In certain embodiments of Formula (I), (II), (III), (IIIa), (IV), (IVa), (V), (Va), (Vb), (VI), (VIa), or (VIb), Z¹ is

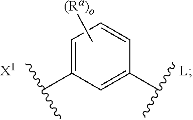

### and Z² is

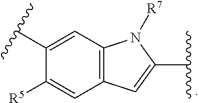

In certain embodiments of Formula (I), (II), (III), (IIIa), (IV), (IVa), (V), (Va), (Vb), (VI), (VIa), or (VIb), Z¹ is

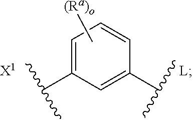

### and Z² is

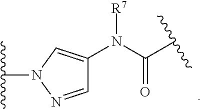

In certain embodiments of Formula (I), (II), (III), (IIIa), (IV), (IVa), (V), (Va), (Vb), (VI), (VIa), or (VIb), Z¹ is

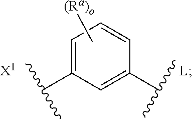

### and Z² is

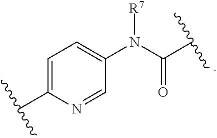

In certain embodiments of Formula (I), (II), (III), (IIIa), (IV), (IVa), (V), (Va), (Vb), (VI), (VIa), or (VIb), Z¹ is

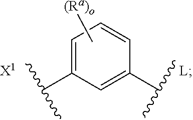

### and Z² is

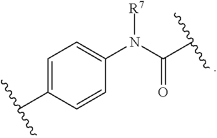

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl. In certain embodiments of this paragraph, R^{a} is halogen, —C₁-C₃ alkyl, or —C₁-C₃ alkaoxy. In certain embodiments of this paragraph, R^{a} is halogen. In certain embodiments of this paragraph, R^{a} is —C₁-C₃ alkyl. In certain embodiments of this paragraph, R^{a} is —C₁-C₃ alkaoxy. In certain embodiments of this paragraph, o is 1, 2, 3, or 4. In certain embodiments of this paragraph, o is 1. In certain embodiments of this paragraph, o is 2. In certain embodiments of this paragraph, o is 3. In certain embodiments of this paragraph, o is 4.

In certain embodiments of Formula (I), (II), (III), (IIIa), (IV), (IVa), (V), (Va), (Vb), (VI), (VIa), or (VIb), Z¹ is

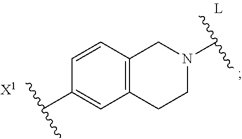

### and Z² is

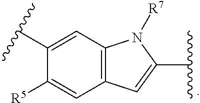

In certain embodiments of Formula (I), (II), (III), (IIIa), (IV), (IVa), (V), (Va), (Vb), (VI), (VIa), or (VIb), Z¹ is

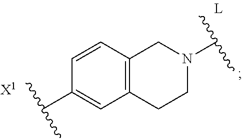

### and Z² is

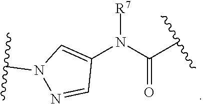

In certain embodiments of Formula (I), (II), (III), (IIIa), (IV), (IVa), (V), (Va), (Vb), (VI), (VIa), or (VIb), Z¹ is

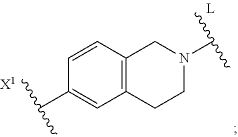

### and Z² is

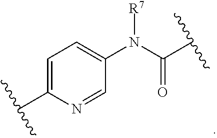

In certain embodiments of Formula (I), (II), (III), (IIIa), (IV), (IVa), (V), (Va), (Vb), (VI), (VIa), or (VIb), Z¹ is

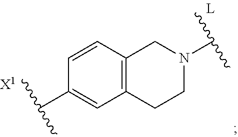

### and Z² is

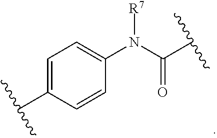

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

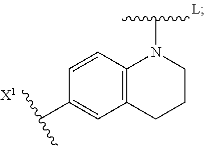

### and Z² is

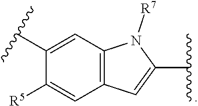

In certain embodiments of Formula (I), (II), (III), (IIIa), (IV), (IVa), (V), (Va), (Vb), (VI), (VIa), or (VIb), Z¹ is

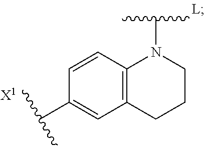

### and Z² is

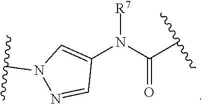

In certain embodiments of Formula (I), (II), (III), (IIIa), (IV), (IVa), (V), (Va), (Vb), (VI), (VIa), or (VIb), Z¹ is

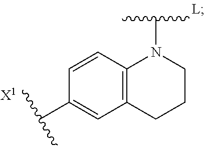

### and Z² is

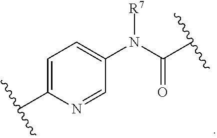

In certain embodiments of Formula (I), (II), (III), (IIIa), (IV), (IVa), (V), (Va), (Vb), (VI), (VIa), or (VIb), Z¹ is

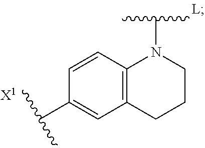

### and Z² is

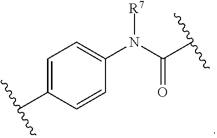

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

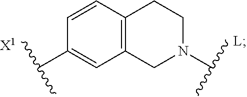

### and Z² is

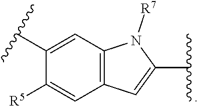

In certain embodiments of Formula (I), (II), (III), (IIIa), (IV), (IVa), (V), (Va), (Vb), (VI), (VIa), or (VIb), Z¹ is

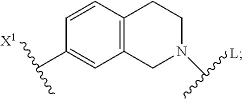

### and Z² is

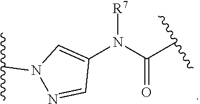

In certain embodiments of Formula (I), (II), (III), (IIIa), (IV), (IVa), (V), (Va), (Vb), (VI), (VIa), or (VIb), Z¹ is

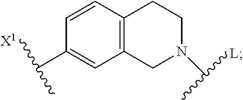

### and Z² is

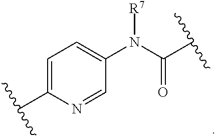

In certain embodiments of Formula (I), (II), (III), (IIIa), (IV), (IVa), (V), (Va), (Vb), (VI), (VIa), or (VIb), Z¹ is

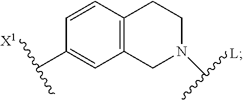

### and Z² is

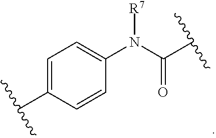

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

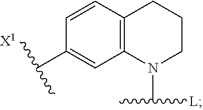

### and Z² is

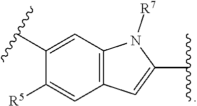

In certain embodiments of Formula (I), (II), (III), (IIIa), (IV), (IVa), (V), (Va), (Vb), (VI), (VIa), or (VIb), Z¹ is

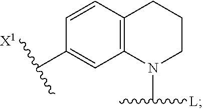

### and Z² is

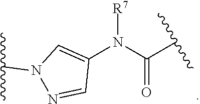

In certain embodiments of Formula (I), (II), (III), (IIIa), (IV), (IVa), (V), (Va), (Vb), (VI), (VIa), or (VIb), Z¹ is

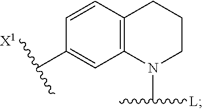

### and Z² is

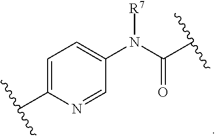

In certain embodiments of Formula (I), (II), (III), (IIIa), (IV), (IVa), (V), (Va), (Vb), (VI), (VIa), or (VIb), Z¹ is

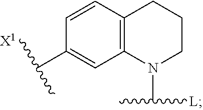

### and Z² is

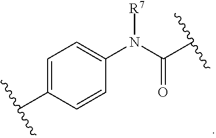

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

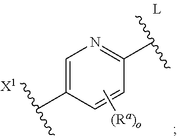

### and Z² is

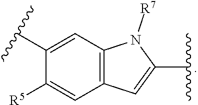

In certain embodiments of Formula (I), (II), (III), (IIIa), (IV), (IVa), (V), (Va), (Vb), (VI), (VIa), or (VIb), Z¹ is

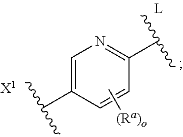

### and Z² is

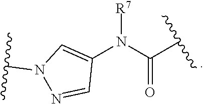

In certain embodiments of Formula (I), (II), (III), (IIIa), (IV), (IVa), (V), (Va), (Vb), (VI), (VIa), or (VIb), Z¹ is

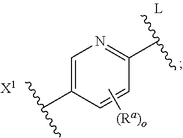

### and Z² is

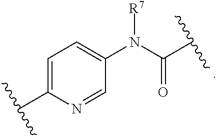

In certain embodiments of Formula (I), (II), (III), (IIIa), (IV), (IVa), (V), (Va), (Vb), (VI), (VIa), or (VIb), Z¹ is

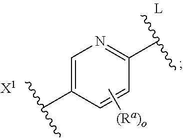

### and Z² is

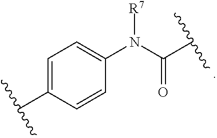

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl. In certain embodiments of this paragraph, R^{a} is halogen, —C₁-C₃ alkyl, or —C₁-C₃ alkaoxy. In certain embodiments of this paragraph, R^{a} is halogen. In certain embodiments of this paragraph, R^{a} is —C₁-C₃ alkyl. In certain embodiments of this paragraph, R^{a} is —C₁-C₃ alkaoxy. In certain embodiments of this paragraph, o is 1, 2, 3, or 4. In certain embodiments of this paragraph, o is 1. In certain embodiments of this paragraph, o is 2. In certain embodiments of this paragraph, o is 3. In certain embodiments of this paragraph, o is 4.

In certain embodiments of Formula (I), (II), (III), (IIIa), (IV), (IVa), (V), (Va), (Vb), (VI), (VIa), or (VIb), Z¹ is

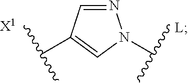

### and Z² is

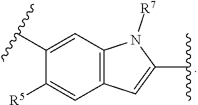

In certain embodiments of Formula (I), (II), (III), (IIIa), (IV), (IVa), (V), (Va), (Vb), (VI), (VIa), or (VIb), Z¹ is

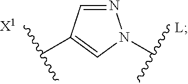

### and Z² is

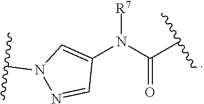

In certain embodiments of Formula (I), (II), (III), (IIIa), (IV), (IVa), (V), (Va), (Vb), (VI), (VIa), or (VIb), Z¹ is

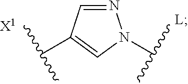

### and Z² is

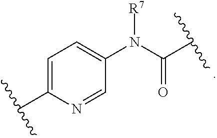

In certain embodiments of Formula (I), (II), (III), (IIIa), (IV), (IVa), (V), (Va), (Vb), (VI), (VIa), or (VIb), Z¹ is

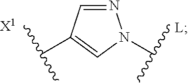

### and Z² is

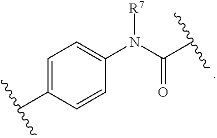

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R^{Z} is hydrogen; R¹⁰ is methyl; X¹ is C—H; R⁵ is hydrogen; and R⁷ is hydrogen. In certain embodiments of this paragraph, R^{Z} is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments of Formula (I), (II), (III), (IIIa), (IV), (IVa), (V), (Va), (Vb), (VI), (VIa), or (VIb), Z¹ is

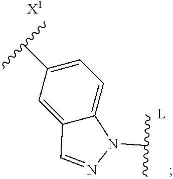

### and Z² is

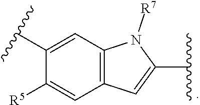

In certain embodiments of Formula (I), (II), (III), (IIIa), (IV), (IVa), (V), (Va), (Vb), (VI), (VIa), or (VIb), Z¹ is

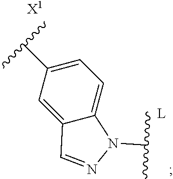

### and Z² is

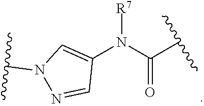

In certain embodiments of Formula (I), (II), (III), (IIIa), (IV), (IVa), (V), (Va), (Vb), (VI), (VIa), or (VIb), Z¹ is

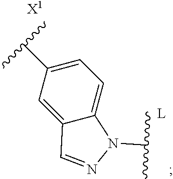

### and Z² is

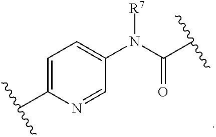

In certain embodiments of Formula (I), (II), (III), (IIIa), (IV), (IVa), (V), (Va), (Vb), (VI), (VIa), or (VIb), Z¹ is

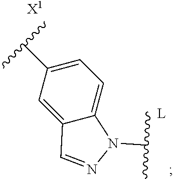

### and Z² is

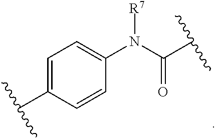

In certain embodiments of this paragraph, R¹ is hydrogen; R¹⁰ is hydrogen; X¹ is C—H; R⁵ is hydrogen; and R⁷ is hydrogen. In certain embodiments of this paragraph, R¹ is hydrogen; R¹⁰ is hydrogen; X¹ is C—H; R⁵ is hydrogen; and R⁷ is methyl. In certain embodiments of this paragraph, R¹ is hydrogen; R¹⁰ is hydrogen; X¹ is C—H; R⁵ is halogen; and R⁷ is hydrogen. In certain embodiments of this paragraph, R¹ is hydrogen; R¹⁰ is hydrogen; X¹ is C—H; R⁵ is halogen; and R⁷ is methyl. In certain embodiments of this paragraph, R¹ is hydrogen; R¹⁰ is hydrogen; X¹ is N; R⁵ is hydrogen; and R⁷ is hydrogen. In certain embodiments of this paragraph, R¹ is hydrogen; R¹⁰ is hydrogen; X¹ is N; R⁵ is hydrogen; and R⁷ is methyl. In certain embodiments of this paragraph, R¹ is hydrogen; R¹⁰ is hydrogen; X¹ is N; R⁵ is halogen; and R⁷ is hydrogen. In certain embodiments of this paragraph, R¹ is hydrogen; R¹⁰ is hydrogen; X¹ is N; R⁵ is halogen; and R⁷ is methyl. In certain embodiments of this paragraph, R¹ is hydrogen; R¹⁰ is methyl; X¹ is C—H; R⁵ is hydrogen; and R⁷ is hydrogen. In certain embodiments of this paragraph, R¹ is hydrogen; R¹⁰ is methyl; X¹ is C—H; R⁵ is hydrogen; and R⁷ is methyl. In certain embodiments of this paragraph, R¹ is hydrogen; R¹⁰ is methyl; X¹ is C—H; R⁵ is halogen; and R⁷ is hydrogen. In certain embodiments of this paragraph, R¹ is hydrogen; R¹⁰ is methyl; X¹ is C—H; R⁵ is halogen; and R⁷ is methyl. In certain embodiments of this paragraph, R¹ is hydrogen; R¹⁰ is methyl; X¹ is N; R⁵ is hydrogen; and R⁷ is hydrogen. In certain embodiments of this paragraph, R¹ is hydrogen; R¹⁰ is methyl; X¹ is N; R⁵ is hydrogen; and R⁷ is methyl. In certain embodiments of this paragraph, R¹ is hydrogen; R¹⁰ is methyl; X¹ is N; R⁵ is halogen; and R⁷ is hydrogen. In certain embodiments of this paragraph, R¹ is hydrogen; R¹⁰ is methyl; X¹ is N; R⁵ is halogen; and R⁷ is methyl. In certain embodiments of this paragraph, R¹ is methyl; R¹⁰ is hydrogen; X¹ is C—H; R⁵ is hydrogen; and R⁷ is hydrogen. In certain embodiments of this paragraph, R¹ is methyl; R¹⁰ is hydrogen; X¹ is C—H; R⁵ is hydrogen; and R⁷ is methyl. In certain embodiments of this paragraph, R¹ is methyl; R¹⁰ is hydrogen; X¹ is C—H; R⁵ is halogen; and R⁷ is hydrogen. In certain embodiments of this paragraph, R¹ is methyl; R¹⁰ is hydrogen; X¹ is C—H; R⁵ is halogen; and R⁷ is methyl. In certain embodiments of this paragraph, R¹ is methyl; R¹⁰ is hydrogen; X¹ is N; R⁵ is hydrogen; and R⁷ is hydrogen. In certain embodiments of this paragraph, R¹ is methyl; R¹⁰ is hydrogen; X¹ is N; R⁵ is hydrogen; and R⁷ is methyl. In certain embodiments of this paragraph, R¹ is methyl; R¹⁰ is hydrogen; X¹ is N; R⁵ is halogen; and R⁷ is hydrogen. In certain embodiments of this paragraph, R¹ is methyl; R¹⁰ is hydrogen; X¹ is N; R⁵ is halogen; and R⁷ is methyl. In certain embodiments of this paragraph, R¹ is methyl; R¹⁰ is methyl; X¹ is C—H; R⁵ is hydrogen; and R⁷ is hydrogen. In certain embodiments of this paragraph, R¹ is methyl; R¹⁰ is methyl; X¹ is C—H; R⁵ is hydrogen; and R⁷ is methyl. In certain embodiments of this paragraph, R¹ is methyl; R¹⁰ is methyl; X¹ is C—H; R⁵ is halogen; and R⁷ is hydrogen. In certain embodiments of this paragraph, R¹ is methyl; R¹⁰ is methyl; X¹ is C—H; R⁵ is halogen; and R⁷ is methyl. In certain embodiments of this paragraph, R¹ is methyl; R¹⁰ is methyl; X¹ is N; R⁵ is hydrogen; and R⁷ is hydrogen. In certain embodiments of this paragraph, R¹ is methyl; R¹⁰ is methyl; X¹ is N; R⁵ is hydrogen; and R⁷ is methyl. In certain embodiments of this paragraph, R¹ is methyl; R¹⁰ is methyl; X¹ is N; R⁵ is halogen; and R⁷ is hydrogen. In certain embodiments of this paragraph, R¹ is methyl; R¹⁰ is methyl; X¹ is N; R⁵ is halogen; and R⁷ is methyl. In certain embodiments of this paragraph, R⁶ is hydrogen. In certain embodiments of this paragraph, R⁶ is methyl.

In certain embodiments, provided herein are compounds of the following Table 1, and stereoisomers and pharmaceutically acceptable salt thereof:

TABLE 1

| Com- | |
|---|---|
| pound | |
| No. | Structure |
| 1 | 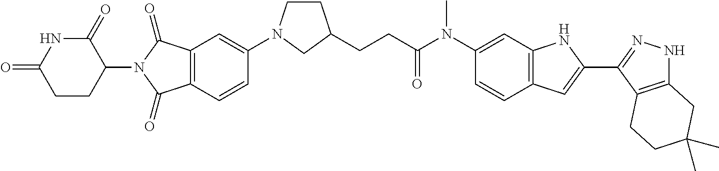 [SMILES: CN(C(=O)CCC1CCN(c2ccc3c(c2)C(=O)N(C2CCC(=O)NC2=O)C3=O)C1)c1ccc2cc(-c3n[nH]c4c3CCC(C)(C)C4)[nH]c2c1] |
| 2 | 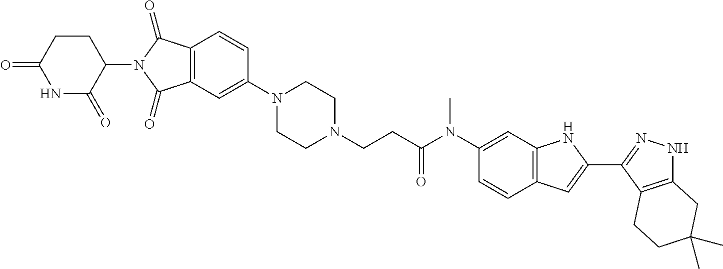 [SMILES: CN(C(=O)CCN1CCN(c2ccc3c(c2)C(=O)N(C2CCC(=O)NC2=O)C3=O)CC1)c1ccc2cc(-c3n[nH]c4c3CCC(C)(C)C4)[nH]c2c1] |
| 3 | 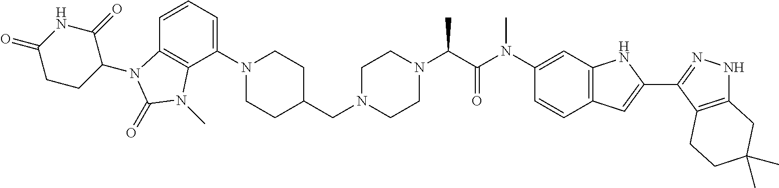 [SMILES: C[C@@H](C(=O)N(C)c1ccc2cc(-c3n[nH]c4c3CCC(C)(C)C4)[nH]c2c1)N1CCN(CC2CCN(c3cccc4c3n(C)c(=O)n4C3CCC(=O)NC3=O)CC2)CC1] |
| 4 | 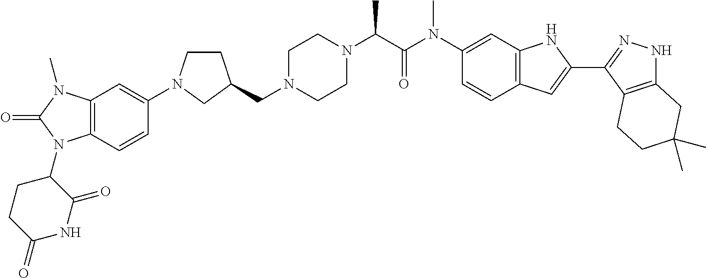 [SMILES: C[C@@H](C(=O)N(C)c1ccc2cc(-c3n[nH]c4c3CCC(C)(C)C4)[nH]c2c1)N1CCN(C[C@@H]2CCN(c3ccc4c(c3)n(C)c(=O)n4C3CCC(=O)NC3=O)C2)CC1] |
| 5 | 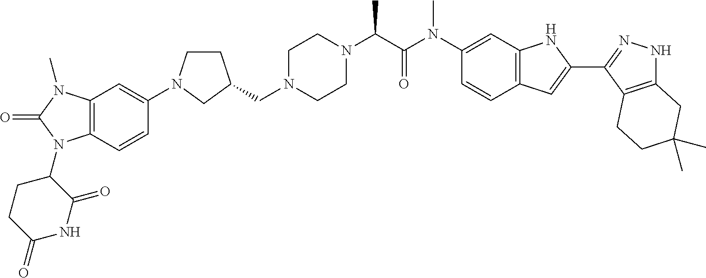 [SMILES: C[C@@H](C(=O)N(C)c1ccc2cc(-c3n[nH]c4c3CCC(C)(C)C4)[nH]c2c1)N1CCN(C[C@H]2CCN(c3ccc4c(c3)n(C)c(=O)n4C3CCC(=O)NC3=O)C2)CC1] |
| 6 | 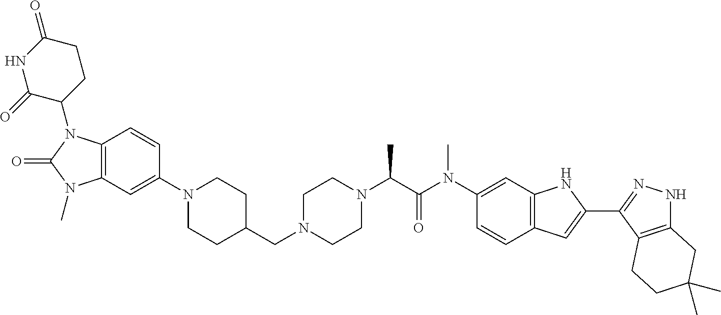 [SMILES: C[C@@H](C(=O)N(C)c1ccc2cc(-c3n[nH]c4c3CCC(C)(C)C4)[nH]c2c1)N1CCN(CC2CCN(c3ccc4c(c3)n(C)c(=O)n4C3CCC(=O)NC3=O)CC2)CC1] |
| 7 | 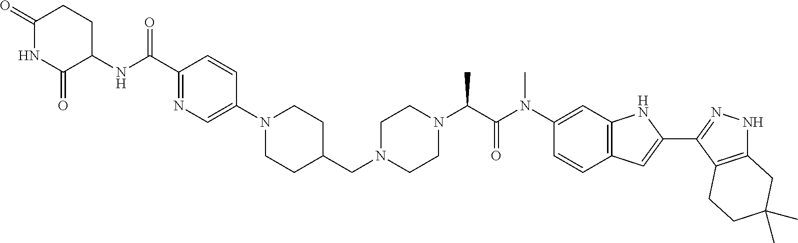 [SMILES: C[C@@H](C(=O)N(C)c1ccc2cc(-c3n[nH]c4c3CCC(C)(C)C4)[nH]c2c1)N1CCN(CC2CCN(c3ccc(C(=O)NC4CCC(=O)NC4=O)nc3)CC2)CC1] |
| 8 | 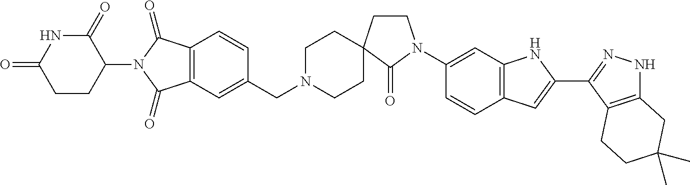 [SMILES: CC1(C)CCc2c(-c3cc4ccc(N5CCC6(CCN(Cc7ccc8c(c7)C(=O)N(C7CCC(=O)NC7=O)C8=O)CC6)C5=O)cc4[nH]3)n[nH]c2C1] |
| 9 | 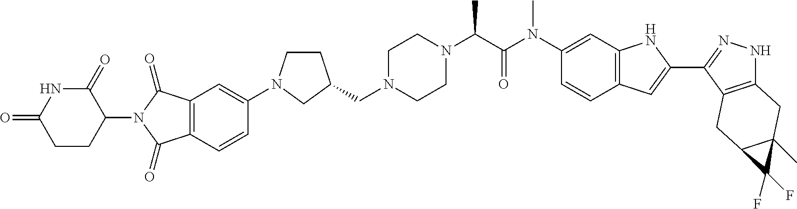 [SMILES: C[C@@H](C(=O)N(C)c1ccc2cc(-c3n[nH]c4c3C[C@@H]3C(F)(F)[C@]3(C)C4)[nH]c2c1)N1CCN(C[C@H]2CCN(c3ccc4c(c3)C(=O)N(C3CCC(=O)NC3=O)C4=O)C2)CC1] |
| 10 | 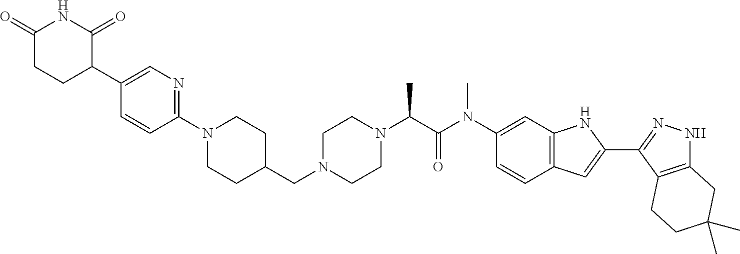 [SMILES: C[C@@H](C(=O)N(C)c1ccc2cc(-c3n[nH]c4c3CCC(C)(C)C4)[nH]c2c1)N1CCN(CC2CCN(c3ccc(C4CCC(=O)NC4=O)cn3)CC2)CC1] |
| 11 | 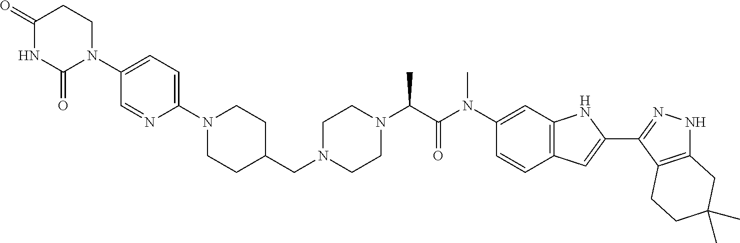 [SMILES: C[C@@H](C(=O)N(C)c1ccc2cc(-c3n[nH]c4c3CCC(C)(C)C4)[nH]c2c1)N1CCN(CC2CCN(c3ccc(N4CCC(=O)NC4=O)cn3)CC2)CC1] |
| 12 | 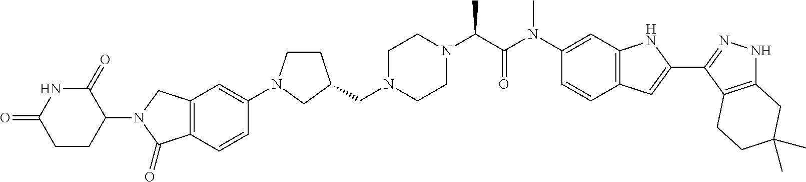 [SMILES: C[C@@H](C(=O)N(C)c1ccc2cc(-c3n[nH]c4c3CCC(C)(C)C4)[nH]c2c1)N1CCN(C[C@H]2CCN(c3ccc4c(c3)CN(C3CCC(=O)NC3=O)C4=O)C2)CC1] |
| 13 | 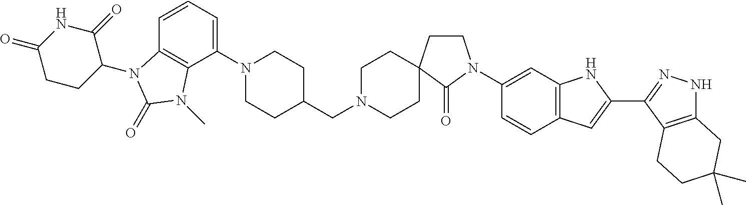 [SMILES: Cn1c(=O)n(C2CCC(=O)NC2=O)c2cccc(N3CCC(CN4CCC5(CC4)CCN(c4ccc6cc(-c7n[nH]c8c7CCC(C)(C)C8)[nH]c6c4)C5=O)CC3)c21] |
| 14 | 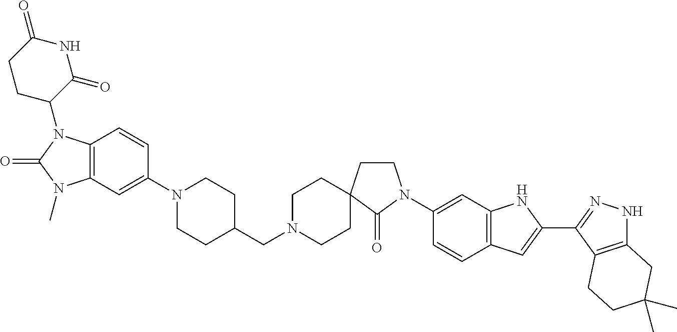 [SMILES: Cn1c(=O)n(C2CCC(=O)NC2=O)c2ccc(N3CCC(CN4CCC5(CC4)CCN(c4ccc6cc(-c7n[nH]c8c7CCC(C)(C)C8)[nH]c6c4)C5=O)CC3)cc21] |
| 15 | 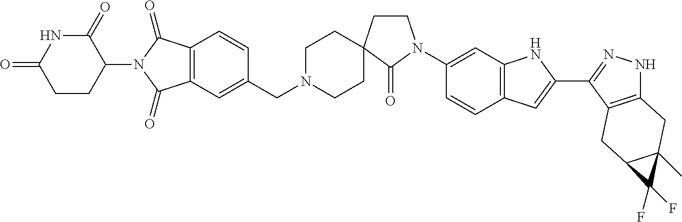 [SMILES: C[C@@]12Cc3[nH]nc(-c4cc5ccc(N6CCC7(CCN(Cc8ccc9c(c8)C(=O)N(C8CCC(=O)NC8=O)C9=O)CC7)C6=O)cc5[nH]4)c3C[C@@H]1C2(F)F] |
| 16 | 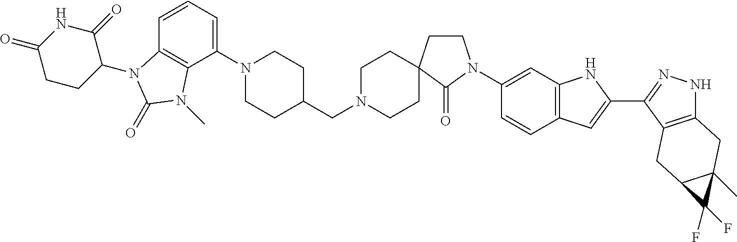 [SMILES: Cn1c(=O)n(C2CCC(=O)NC2=O)c2cccc(N3CCC(CN4CCC5(CC4)CCN(c4ccc6cc(-c7n[nH]c8c7C[C@@H]7C(F)(F)[C@]7(C)C8)[nH]c6c4)C5=O)CC3)c21] |
| 17 | 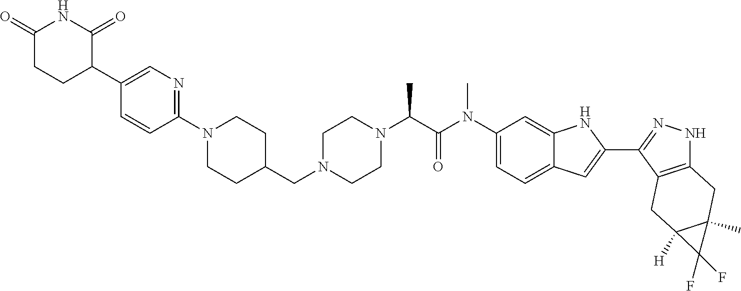 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(C4CCC(=O)NC4=O)cn3)CC2)CC1] |
| 18 | 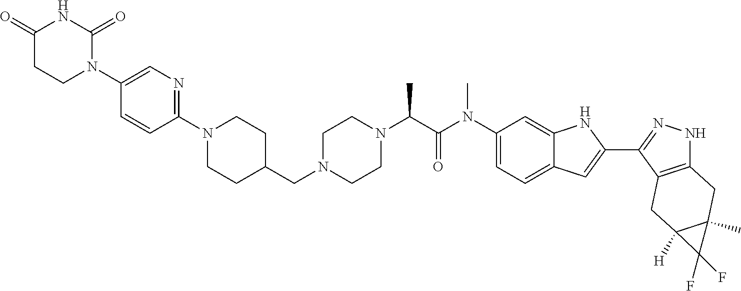 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(N4CCC(=O)NC4=O)cn3)CC2)CC1] |
| 19 | 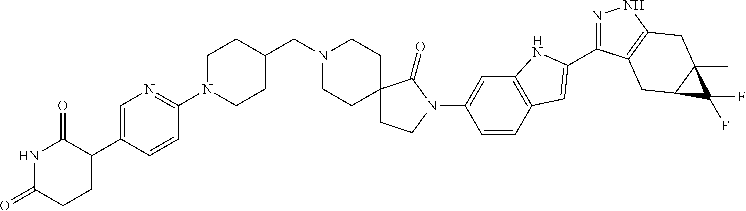 [SMILES: C[C@@]12Cc3[nH]nc(-c4cc5ccc(N6CCC7(CCN(CC8CCN(c9ccc(C%10CCC(=O)NC%10=O)cn9)CC8)CC7)C6=O)cc5[nH]4)c3C[C@@H]1C2(F)F] |
| 20 | 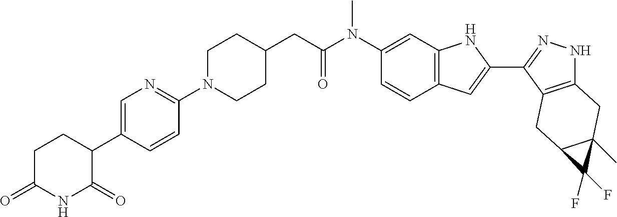 [SMILES: CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1] |
| 21 | 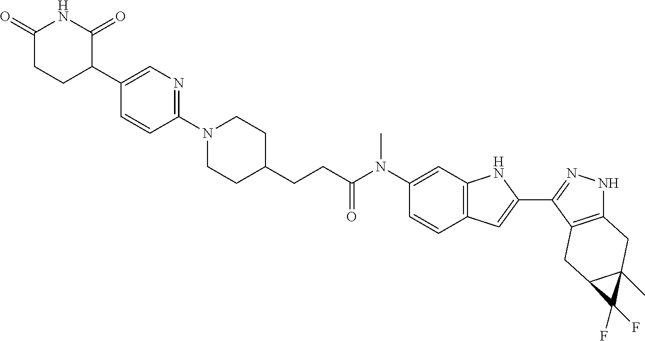 [SMILES: CN(C(=O)CCC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1] |
| 22 | 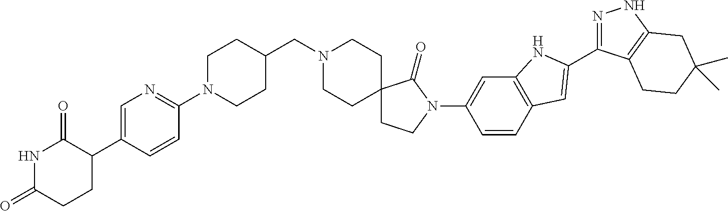 [SMILES: CC1(C)CCc2c(-c3cc4ccc(N5CCC6(CCN(CC7CCN(c8ccc(C9CCC(=O)NC9=O)cn8)CC7)CC6)C5=O)cc4[nH]3)n[nH]c2C1] |
| 23 | 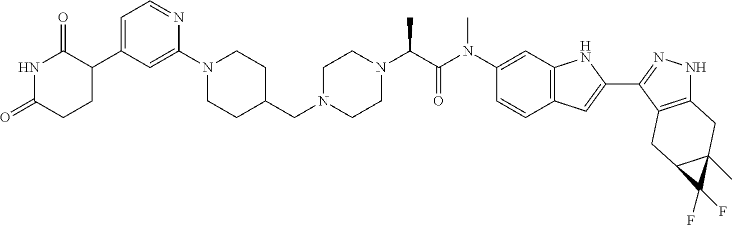 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3cc(C4CCC(=O)NC4=O)ccn3)CC2)CC1] |
| 24 | 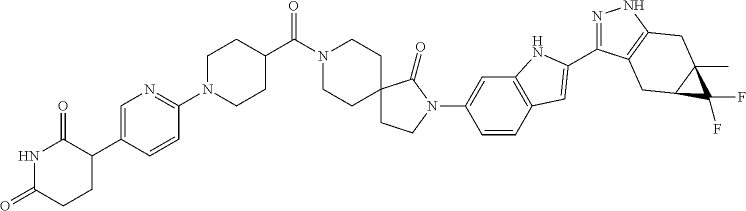 [SMILES: C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F] |
| 25 | 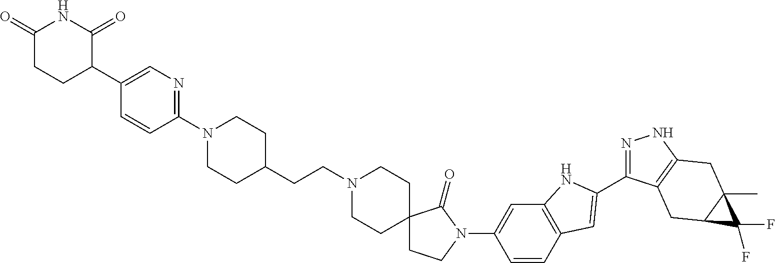 [SMILES: C[C@@]12Cc3[nH]nc(-c4cc5ccc(N6CCC7(CCN(CCC8CCN(c9ccc(C%10CCC(=O)NC%10=O)cn9)CC8)CC7)C6=O)cc5[nH]4)c3C[C@@H]1C2(F)F] |
| 26 | 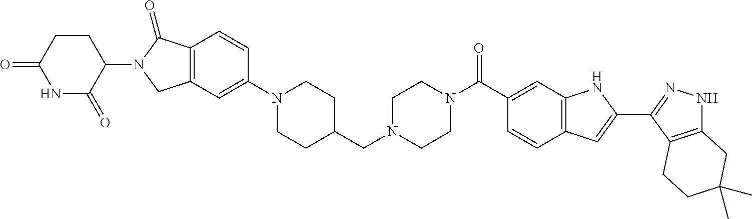 [SMILES: CC1(C)CCc2c(-c3cc4ccc(C(=O)N5CCN(CC6CCN(c7ccc8c(c7)CN(C7CCC(=O)NC7=O)C8=O)CC6)CC5)cc4[nH]3)n[nH]c2C1] |
| 27 | 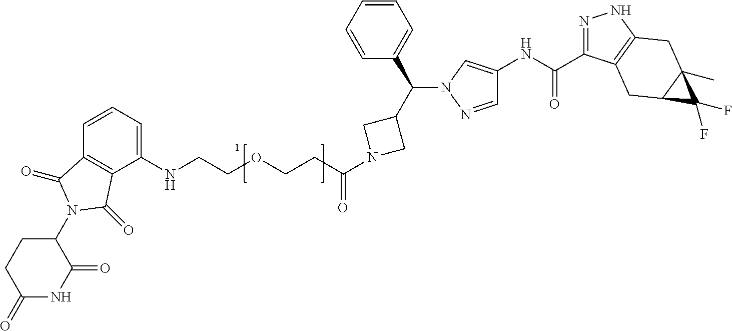 |
| 28 | 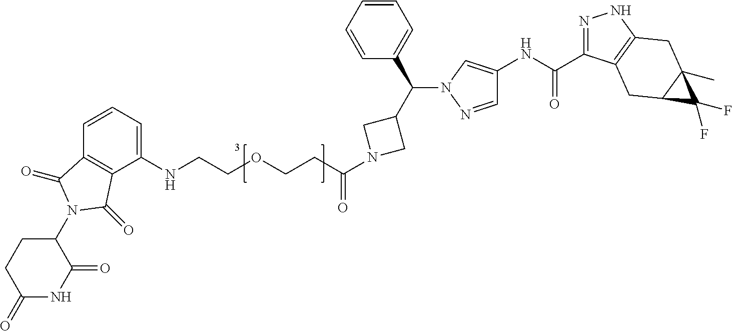 |
| 29 | 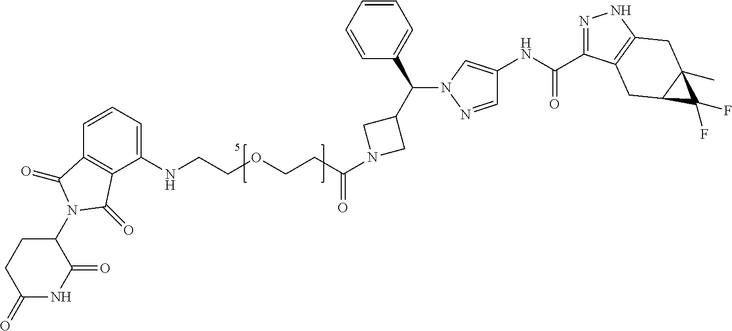 |
| 30 | 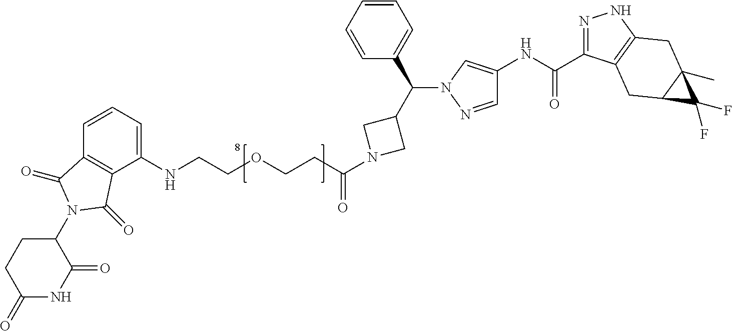 |
| 31 | 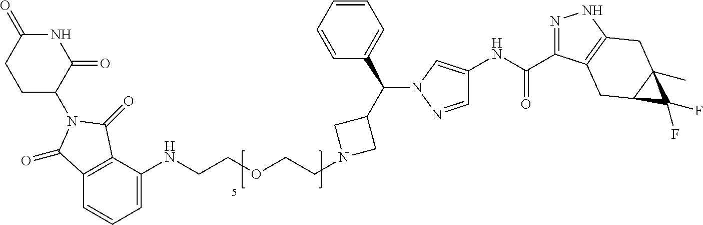 |
| 32 | 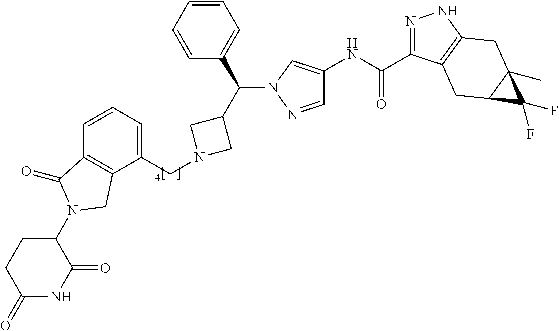 |
| 33 | 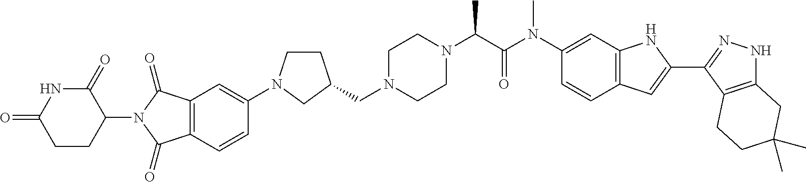 [SMILES: C[C@@H](C(=O)N(C)c1ccc2cc(-c3n[nH]c4c3CCC(C)(C)C4)[nH]c2c1)N1CCN(C[C@H]2CCN(c3ccc4c(c3)C(=O)N(C3CCC(=O)NC3=O)C4=O)C2)CC1] |
| 34 | 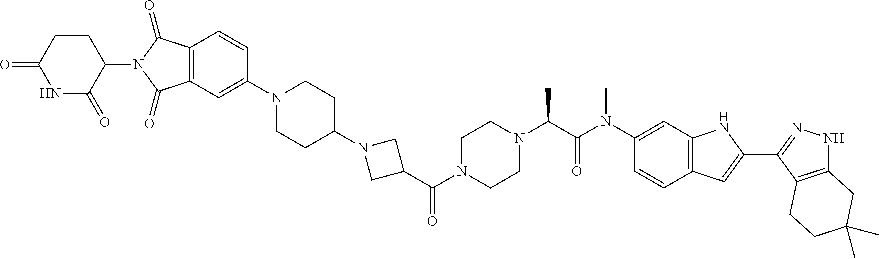 [SMILES: C[C@@H](C(=O)N(C)c1ccc2cc(-c3n[nH]c4c3CCC(C)(C)C4)[nH]c2c1)N1CCN(C(=O)C2CN(C3CCN(c4ccc5c(c4)C(=O)N(C4CCC(=O)NC4=O)C5=O)CC3)C2)CC1] |
| 35 | 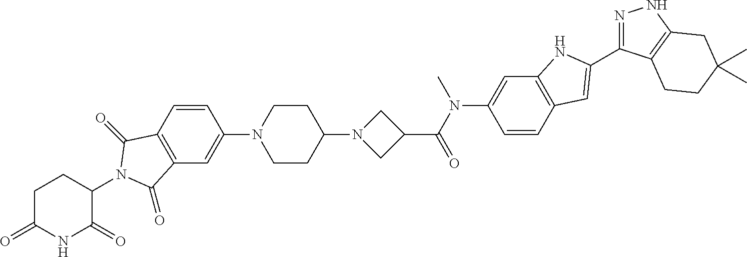 [SMILES: CN(C(=O)C1CN(C2CCN(c3ccc4c(c3)C(=O)N(C3CCC(=O)NC3=O)C4=O)CC2)C1)c1ccc2cc(-c3n[nH]c4c3CCC(C)(C)C4)[nH]c2c1] |
| 36 | 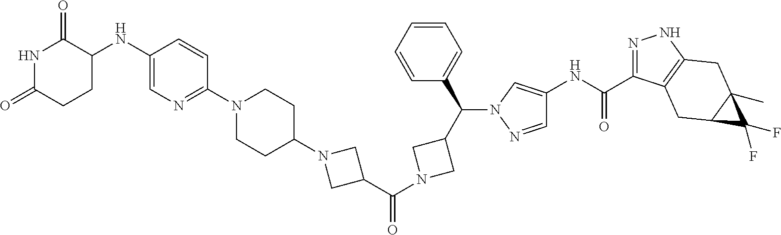 [SMILES: C[C@@]12Cc3[nH]nc(C(=O)Nc4cnn([C@H](c5ccccc5)C5CN(C(=O)C6CN(C7CCN(c8ccc(NC9CCC(=O)NC9=O)cn8)CC7)C6)C5)c4)c3C[C@@H]1C2(F)F] |
| 37 | 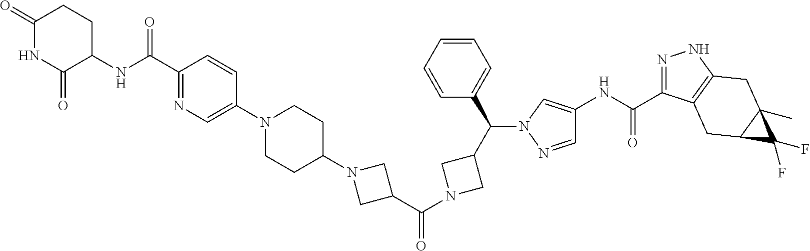 [SMILES: C[C@@]12Cc3[nH]nc(C(=O)Nc4cnn([C@H](c5ccccc5)C5CN(C(=O)C6CN(C7CCN(c8ccc(C(=O)NC9CCC(=O)NC9=O)nc8)CC7)C6)C5)c4)c3C[C@@H]1C2(F)F] |
| 38 | 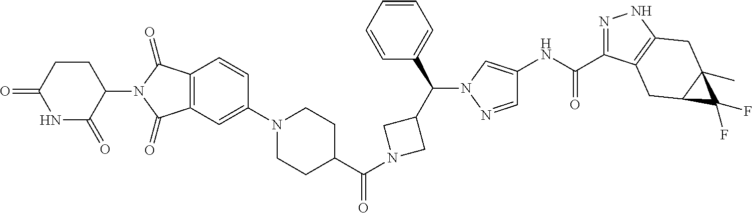 [SMILES: C[C@@]12Cc3[nH]nc(C(=O)Nc4cnn([C@H](c5ccccc5)C5CN(C(=O)C6CCN(c7ccc8c(c7)C(=O)N(C7CCC(=O)NC7=O)C8=O)CC6)C5)c4)c3C[C@@H]1C2(F)F] |
| 39 | 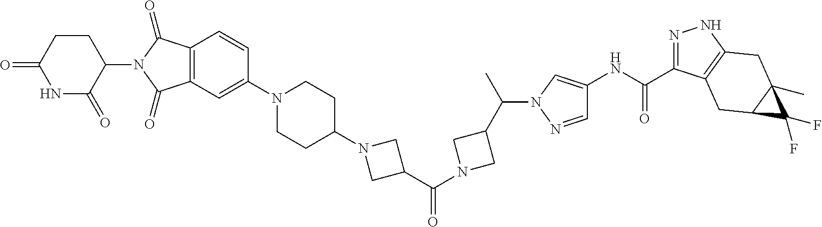 [SMILES: CC(C1CN(C(=O)C2CN(C3CCN(c4ccc5c(c4)C(=O)N(C4CCC(=O)NC4=O)C5=O)CC3)C2)C1)n1cc(NC(=O)c2n[nH]c3c2C[C@@H]2C(F)(F)[C@]2(C)C3)cn1] |
| 40 | 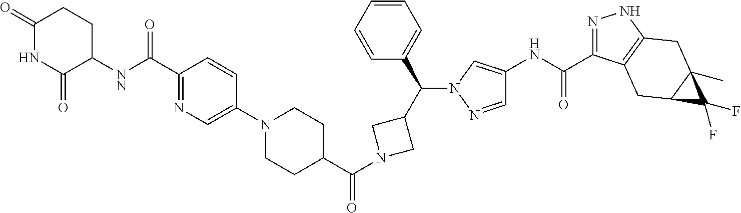 |
| 41 | 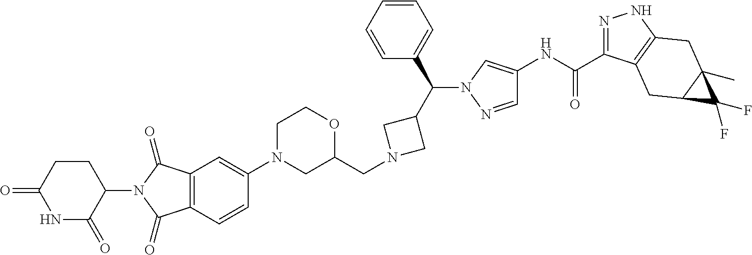 [SMILES: C[C@@]12Cc3[nH]nc(C(=O)Nc4cnn([C@H](c5ccccc5)C5CN(CC6CN(c7ccc8c(c7)C(=O)N(C7CCC(=O)NC7=O)C8=O)CCO6)C5)c4)c3C[C@@H]1C2(F)F] |
| 42 | 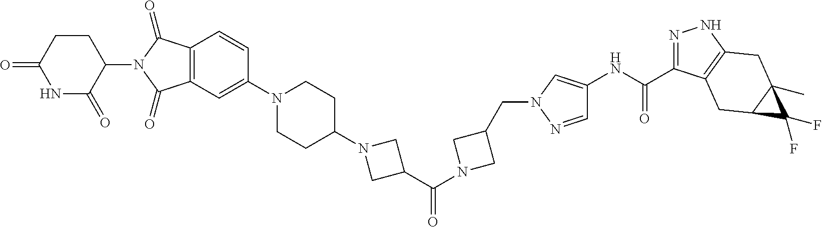 [SMILES: C[C@@]12Cc3[nH]nc(C(=O)Nc4cnn(CC5CN(C(=O)C6CN(C7CCN(c8ccc9c(c8)C(=O)N(C8CCC(=O)NC8=O)C9=O)CC7)C6)C5)c4)c3C[C@@H]1C2(F)F] |
| 43 | 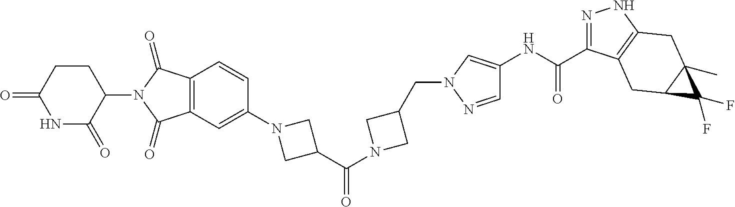 [SMILES: C[C@@]12Cc3[nH]nc(C(=O)Nc4cnn(CC5CN(C(=O)C6CN(c7ccc8c(c7)C(=O)N(C7CCC(=O)NC7=O)C8=O)C6)C5)c4)c3C[C@@H]1C2(F)F] |
| 44 | 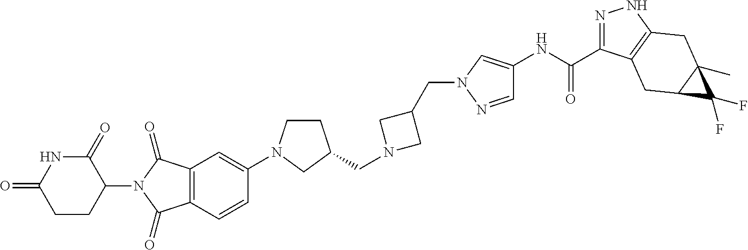 [SMILES: C[C@@]12Cc3[nH]nc(C(=O)Nc4cnn(CC5CN(C[C@H]6CCN(c7ccc8c(c7)C(=O)N(C7CCC(=O)NC7=O)C8=O)C6)C5)c4)c3C[C@@H]1C2(F)F] |
| 45 | 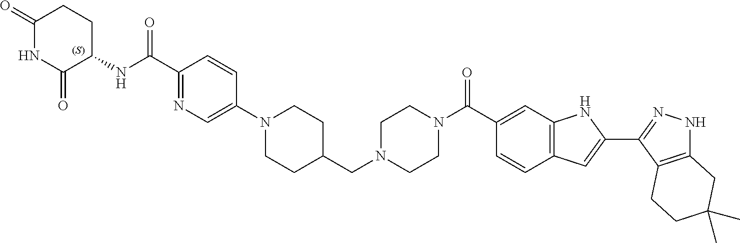 |
| 46 | 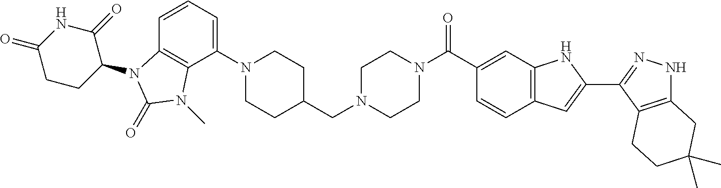 [SMILES: Cn1c(=O)n([C@H]2CCC(=O)NC2=O)c2cccc(N3CCC(CN4CCN(C(=O)c5ccc6cc(-c7n[nH]c8c7CCC(C)(C)C8)[nH]c6c5)CC4)CC3)c21] |
| 47 | 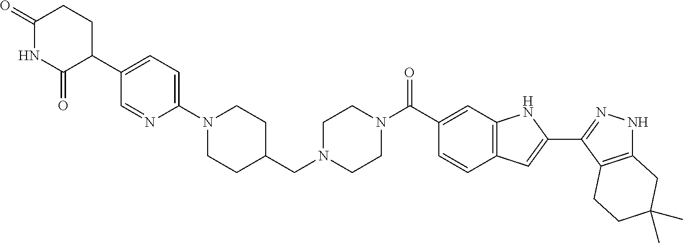 [SMILES: CC1(C)CCc2c(-c3cc4ccc(C(=O)N5CCN(CC6CCN(c7ccc(C8CCC(=O)NC8=O)cn7)CC6)CC5)cc4[nH]3)n[nH]c2C1] |
| 48 | 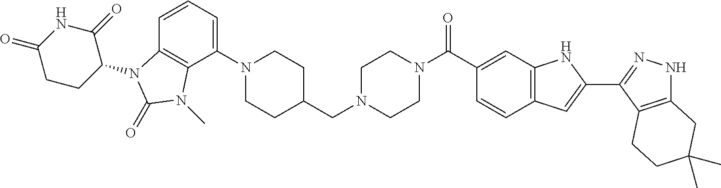 [SMILES: Cn1c(=O)n([C@@H]2CCC(=O)NC2=O)c2cccc(N3CCC(CN4CCN(C(=O)c5ccc6cc(-c7n[nH]c8c7CCC(C)(C)C8)[nH]c6c5)CC4)CC3)c21] |
| 49 | 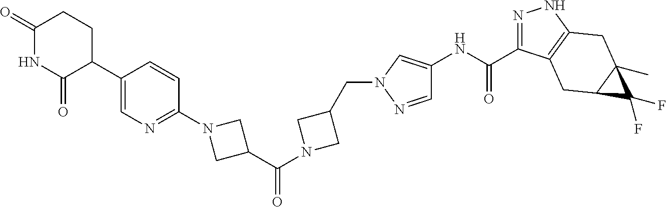 [SMILES: C[C@@]12Cc3[nH]nc(C(=O)Nc4cnn(CC5CN(C(=O)C6CN(c7ccc(C8CCC(=O)NC8=O)cn7)C6)C5)c4)c3C[C@@H]1C2(F)F] |
| 50 | 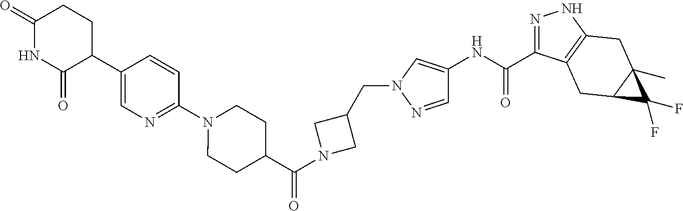 [SMILES: C[C@@]12Cc3[nH]nc(C(=O)Nc4cnn(CC5CN(C(=O)C6CCN(c7ccc(C8CCC(=O)NC8=O)cn7)CC6)C5)c4)c3C[C@@H]1C2(F)F] |
| 51 | 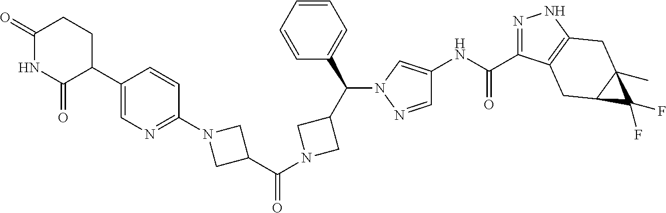 [SMILES: C[C@@]12Cc3[nH]nc(C(=O)Nc4cnn([C@H](c5ccccc5)C5CN(C(=O)C6CN(c7ccc(C8CCC(=O)NC8=O)cn7)C6)C5)c4)c3C[C@@H]1C2(F)F] |
| 52 | 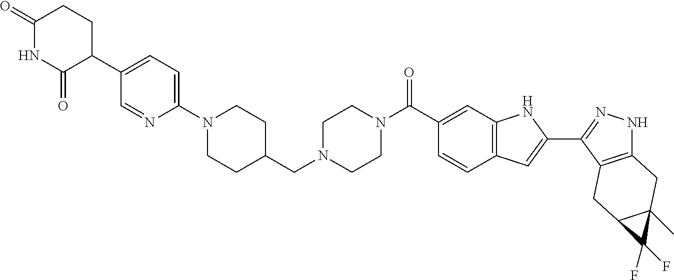 [SMILES: C[C@@]12Cc3[nH]nc(-c4cc5ccc(C(=O)N6CCN(CC7CCN(c8ccc(C9CCC(=O)NC9=O)cn8)CC7)CC6)cc5[nH]4)c3C[C@@H]1C2(F)F] |
| 53 | 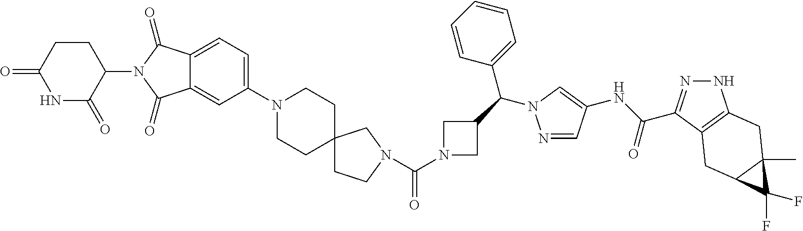 [SMILES: C[C@@]12Cc3[nH]nc(C(=O)Nc4cnn([C@H](c5ccccc5)C5CN(C(=O)N6CCC7(CCN(c8ccc9c(c8)C(=O)N(C8CCC(=O)NC8=O)C9=O)CC7)C6)C5)c4)c3C[C@@H]1C2(F)F] |
| 54 | 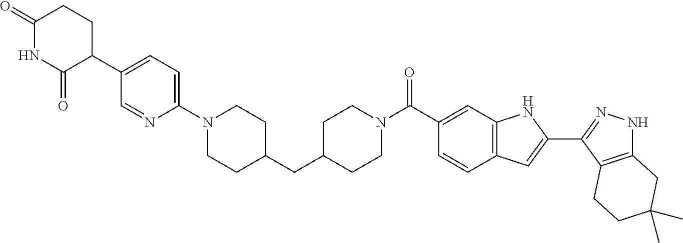 [SMILES: CC1(C)CCc2c(-c3cc4ccc(C(=O)N5CCC(CC6CCN(c7ccc(C8CCC(=O)NC8=O)cn7)CC6)CC5)cc4[nH]3)n[nH]c2C1] |
| 55 | 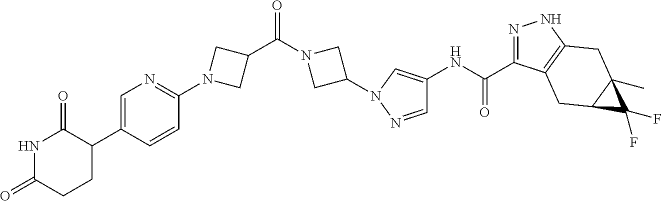 [SMILES: C[C@@]12Cc3[nH]nc(C(=O)Nc4cnn(C5CN(C(=O)C6CN(c7ccc(C8CCC(=O)NC8=O)cn7)C6)C5)c4)c3C[C@@H]1C2(F)F] |
| 56 | 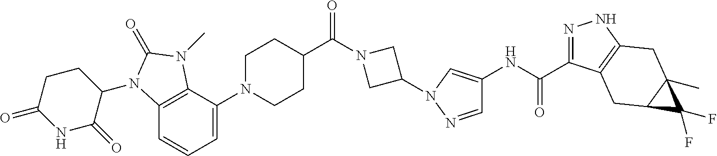 [SMILES: Cn1c(=O)n(C2CCC(=O)NC2=O)c2cccc(N3CCC(C(=O)N4CC(n5cc(NC(=O)c6n[nH]c7c6C[C@@H]6C(F)(F)[C@]6(C)C7)cn5)C4)CC3)c21] |
| 57 | 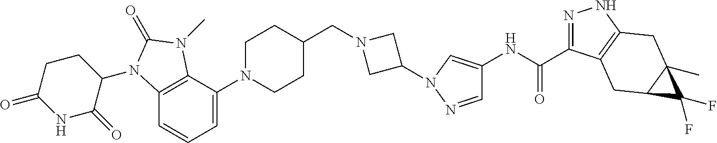 [SMILES: Cn1c(=O)n(C2CCC(=O)NC2=O)c2cccc(N3CCC(CN4CC(n5cc(NC(=O)c6n[nH]c7c6C[C@@H]6C(F)(F)[C@]6(C)C7)cn5)C4)CC3)c21] |
| 58 | 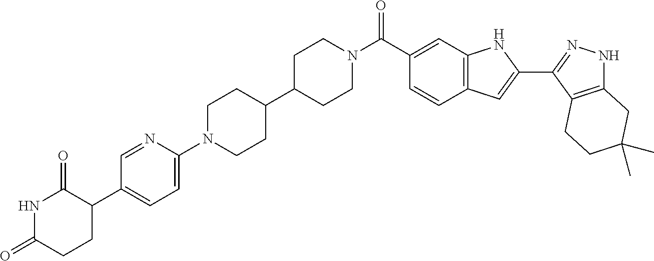 [SMILES: CC1(C)CCc2c(-c3cc4ccc(C(=O)N5CCC(C6CCN(c7ccc(C8CCC(=O)NC8=O)cn7)CC6)CC5)cc4[nH]3)n[nH]c2C1] |
| 59 | 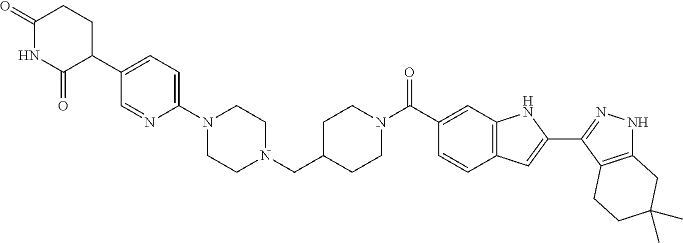 [SMILES: CC1(C)CCc2c(-c3cc4ccc(C(=O)N5CCC(CN6CCN(c7ccc(C8CCC(=O)NC8=O)cn7)CC6)CC5)cc4[nH]3)n[nH]c2C1] |
| 60 | 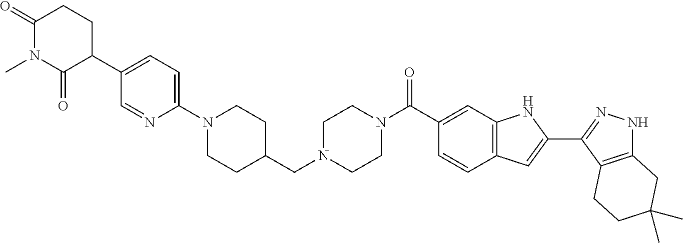 [SMILES: CN1C(=O)CCC(c2ccc(N3CCC(CN4CCN(C(=O)c5ccc6cc(-c7n[nH]c8c7CCC(C)(C)C8)[nH]c6c5)CC4)CC3)nc2)C1=O] |
| 61 | 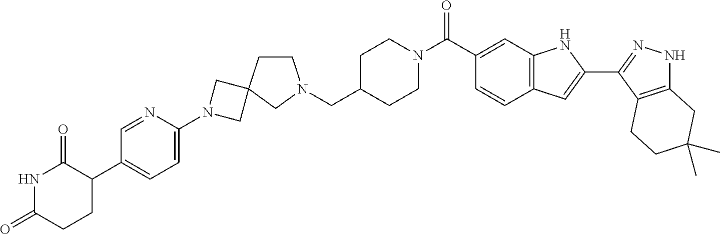 [SMILES: CC1(C)CCc2c(-c3cc4ccc(C(=O)N5CCC(CN6CCC7(C6)CN(c6ccc(C8CCC(=O)NC8=O)cn6)C7)CC5)cc4[nH]3)n[nH]c2C1] |
| 62 | 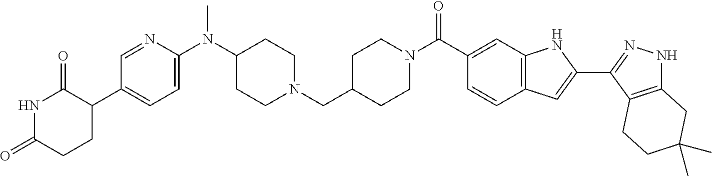 [SMILES: CN(c1ccc(C2CCC(=O)NC2=O)cn1)C1CCN(CC2CCN(C(=O)c3ccc4cc(-c5n[nH]c6c5CCC(C)(C)C6)[nH]c4c3)CC2)CC1] |
| 63 | 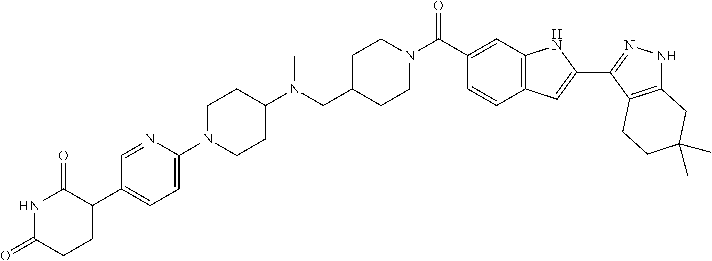 [SMILES: CN(CC1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1] |
| 64 | 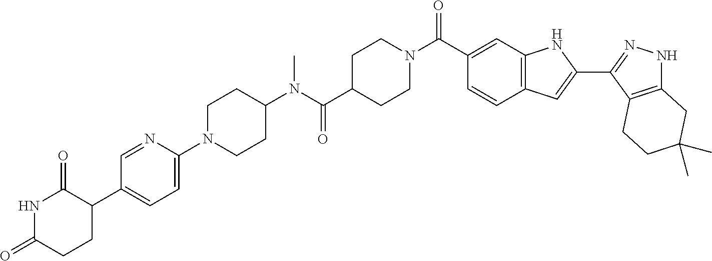 [SMILES: CN(C(=O)C1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1] |
| 65 | 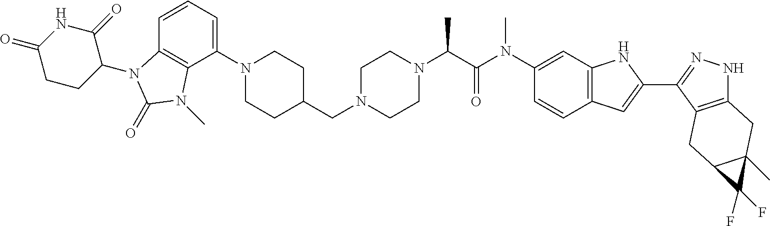 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3cccc4c3n(C)c(=O)n4C3CCC(=O)NC3=O)CC2)CC1] |
| 66 | 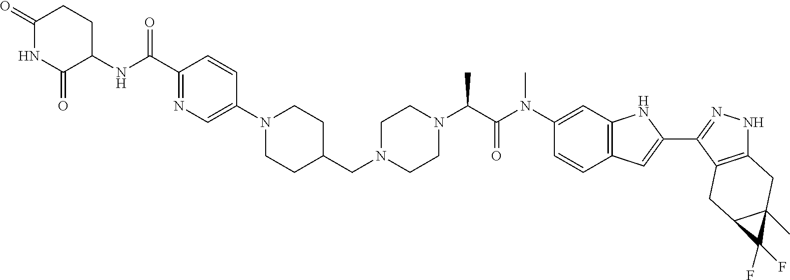 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(C(=O)NC4CCC(=O)NC4=O)nc3)CC2)CC1] |
| 67 | 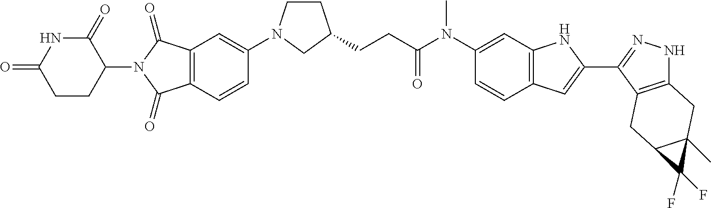 [SMILES: CN(C(=O)CC[C@H]1CCN(c2ccc3c(c2)C(=O)N(C2CCC(=O)NC2=O)C3=O)C1)c1ccc2cc(-c3n[nH]c4c3C[C@@H]3C(F)(F)[C@]3(C)C4)[nH]c2c1] |
| 68 | 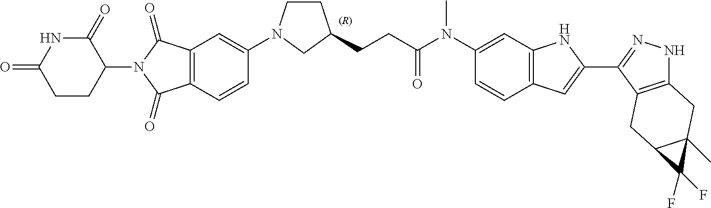 |
| 69 | 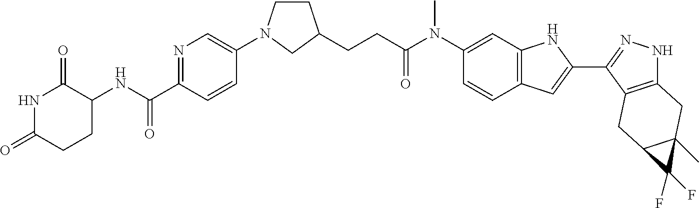 [SMILES: CN(C(=O)CCC1CCN(c2ccc(C(=O)NC3CCC(=O)NC3=O)nc2)C1)c1ccc2cc(-c3n[nH]c4c3C[C@@H]3C(F)(F)[C@]3(C)C4)[nH]c2c1] |
| 70 | 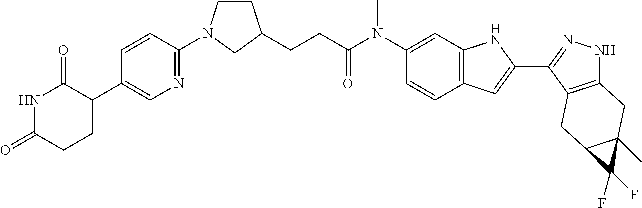 [SMILES: CN(C(=O)CCC1CCN(c2ccc(C3CCC(=O)NC3=O)cn2)C1)c1ccc2cc(-c3n[nH]c4c3C[C@@H]3C(F)(F)[C@]3(C)C4)[nH]c2c1] |
| 71 | 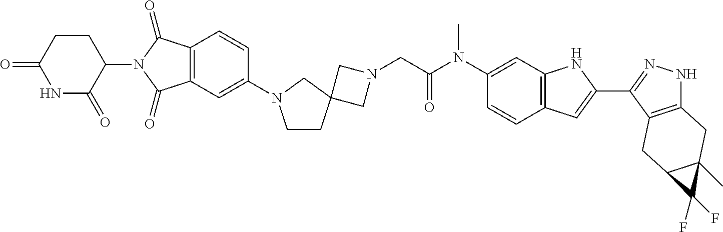 [SMILES: CN(C(=O)CN1CC2(CCN(c3ccc4c(c3)C(=O)N(C3CCC(=O)NC3=O)C4=O)C2)C1)c1ccc2cc(-c3n[nH]c4c3C[C@@H]3C(F)(F)[C@]3(C)C4)[nH]c2c1] |
| 71 | 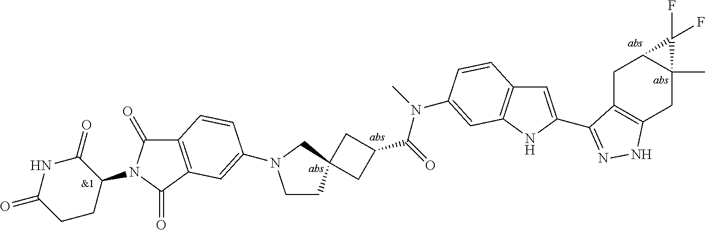 [SMILES: CN(c1ccc2cc(-c3n[nH]c4c3C[C@@H]3C(F)(F)[C@]3(C)C4)[nH]c2c1)C(=O)[C@H]1C[C@@]2(CCN(c3ccc4c(c3)C(=O)N([C@H]3CCC(=O)NC3=O)C4=O)C2)C1] |
| 71 | 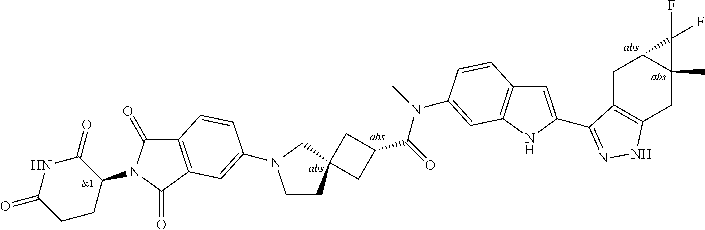 [SMILES: CN(c1ccc2cc(-c3n[nH]c4c3C[C@@H]3C(F)(F)[C@]3(C)C4)[nH]c2c1)C(=O)[C@H]1C[C@]2(CCN(c3ccc4c(c3)C(=O)N([C@H]3CCC(=O)NC3=O)C4=O)C2)C1] |
| 72 | 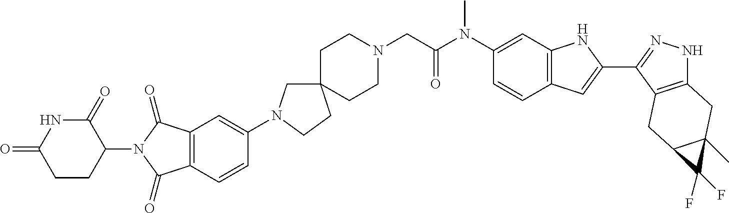 [SMILES: CN(C(=O)CN1CCC2(CC1)CCN(c1ccc3c(c1)C(=O)N(C1CCC(=O)NC1=O)C3=O)C2)c1ccc2cc(-c3n[nH]c4c3C[C@@H]3C(F)(F)[C@]3(C)C4)[nH]c2c1] |
| 73 | 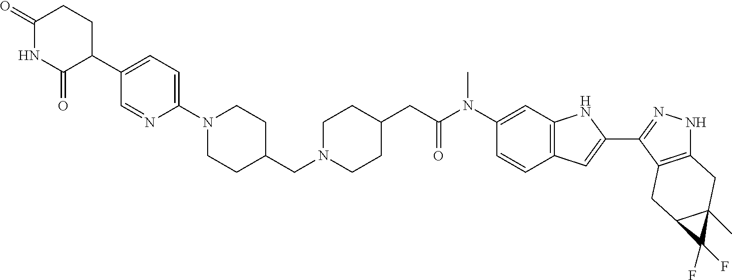 [SMILES: CN(C(=O)CC1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1] |
| 74 | 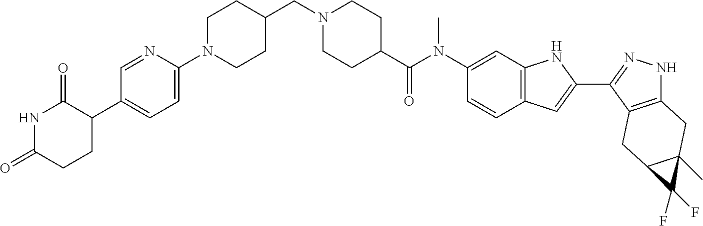 [SMILES: CN(C(=O)C1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1] |
| 75 | 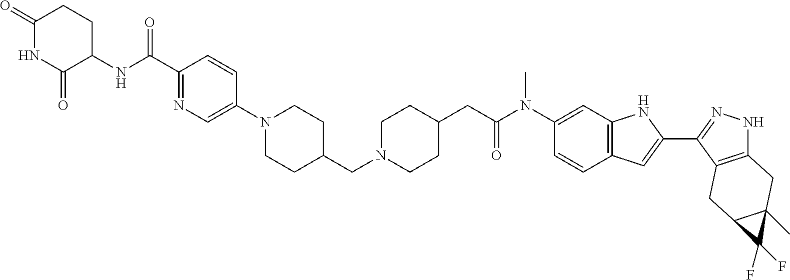 [SMILES: CN(C(=O)CC1CCN(CC2CCN(c3ccc(C(=O)NC4CCC(=O)NC4=O)nc3)CC2)CC1)c1ccc2cc(-c3n[nH]c4c3C[C@@H]3C(F)(F)[C@]3(C)C4)[nH]c2c1] |
| 76 | 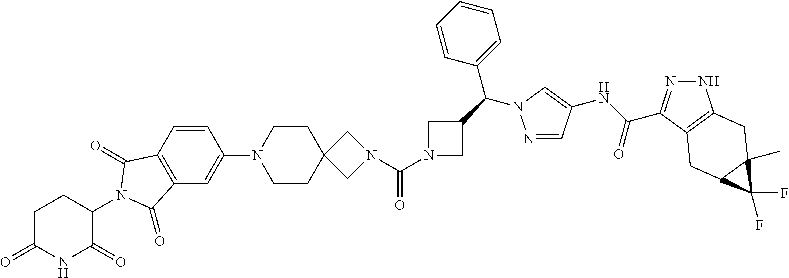 |
| 77 | 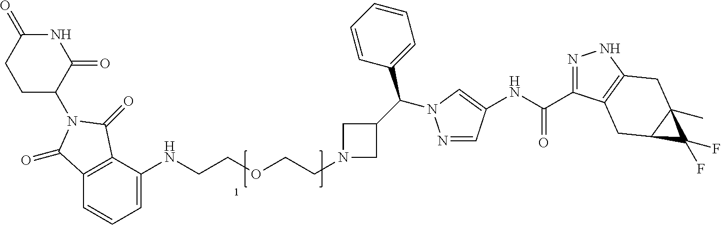 |
| 78 | 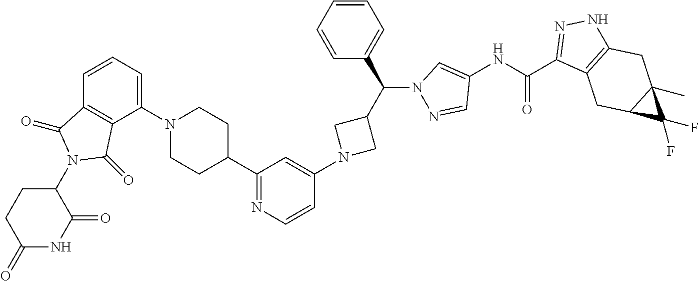 [SMILES: C[C@@]12Cc3[nH]nc(C(=O)Nc4cnn([C@H](c5ccccc5)C5CN(c6ccnc(C7CCN(c8cccc9c8C(=O)N(C8CCC(=O)NC8=O)C9=O)CC7)c6)C5)c4)c3C[C@@H]1C2(F)F] |
| 79 | 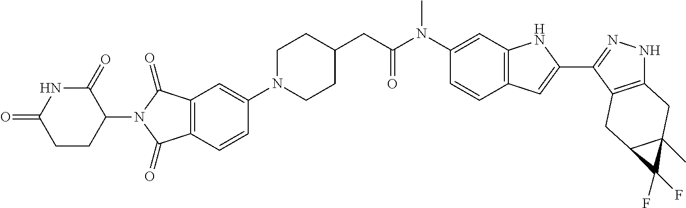 [SMILES: CN(C(=O)CC1CCN(c2ccc3c(c2)C(=O)N(C2CCC(=O)NC2=O)C3=O)CC1)c1ccc2cc(-c3n[nH]c4c3C[C@@H]3C(F)(F)[C@]3(C)C4)[nH]c2c1] |
| 80 | 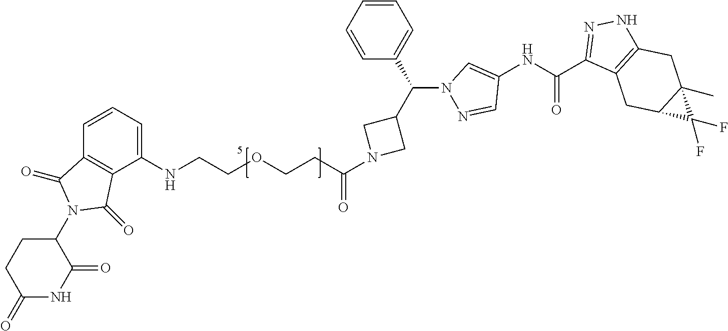 |
| 81 | 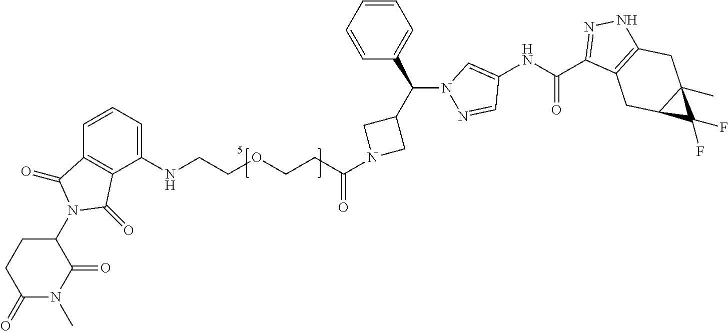 |
| 82 | 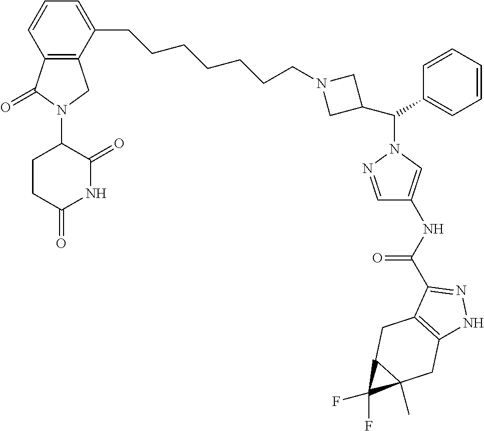 |
| 83 | 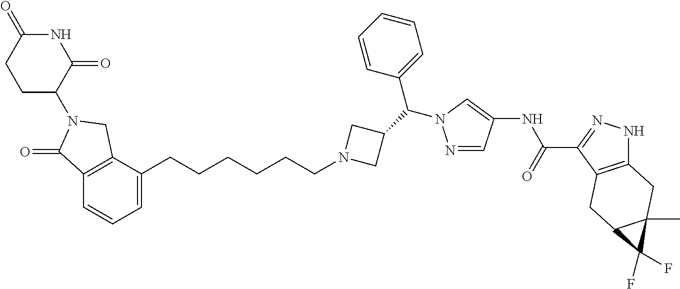 |
| 84 | 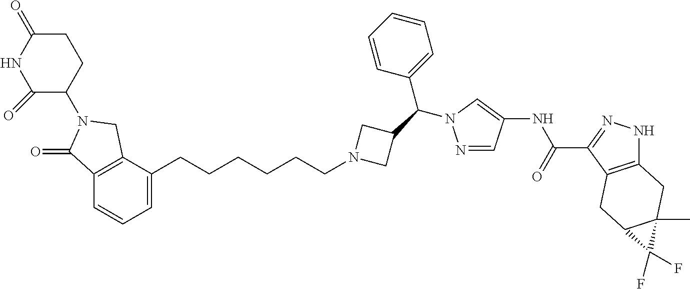 |
| 85 | 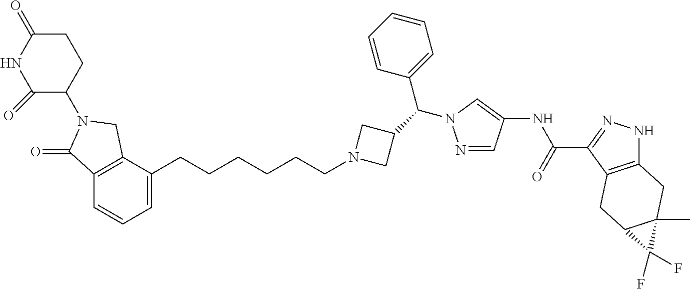 |
| 86 | 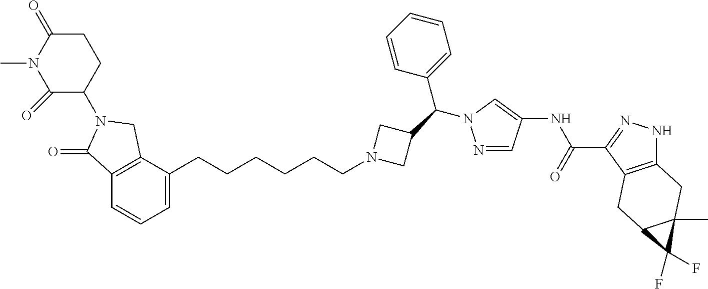 |
| 87 | 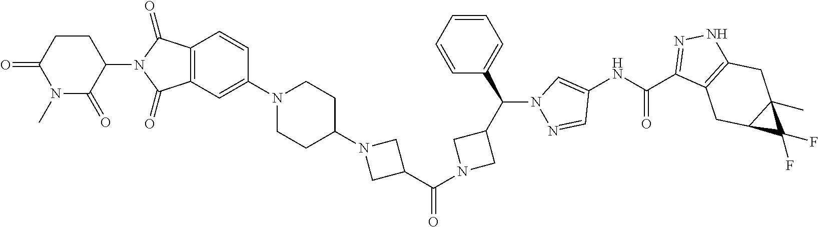 [SMILES: CN1C(=O)CCC(N2C(=O)c3ccc(N4CCC(N5CC(C(=O)N6CC([C@@H](c7ccccc7)n7cc(NC(=O)c8n[nH]c9c8C[C@@H]8C(F)(F)[C@]8(C)C9)cn7)C6)C5)CC4)cc3C2=O)C1=O] |
| 88 | 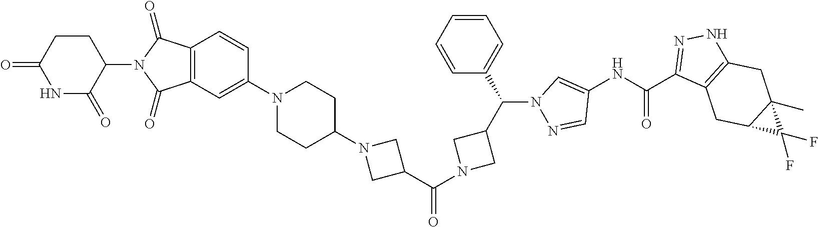 [SMILES: C[C@]12Cc3[nH]nc(C(=O)Nc4cnn([C@@H](c5ccccc5)C5CN(C(=O)C6CN(C7CCN(c8ccc9c(c8)C(=O)N(C8CCC(=O)NC8=O)C9=O)CC7)C6)C5)c4)c3C[C@H]1C2(F)F] |
| 89 | 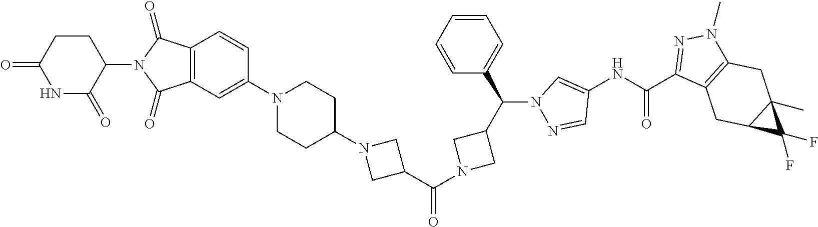 [SMILES: Cn1nc(C(=O)Nc2cnn([C@H](c3ccccc3)C3CN(C(=O)C4CN(C5CCN(c6ccc7c(c6)C(=O)N(C6CCC(=O)NC6=O)C7=O)CC5)C4)C3)c2)c2c1C[C@]1(C)[C@H](C2)C1(F)F] |
| 90 | 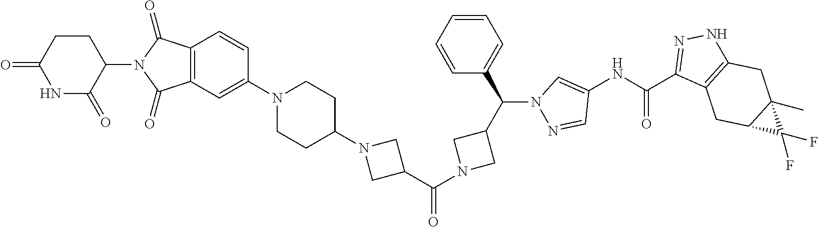 [SMILES: C[C@]12Cc3[nH]nc(C(=O)Nc4cnn([C@H](c5ccccc5)C5CN(C(=O)C6CN(C7CCN(c8ccc9c(c8)C(=O)N(C8CCC(=O)NC8=O)C9=O)CC7)C6)C5)c4)c3C[C@H]1C2(F)F] |
| 91 | 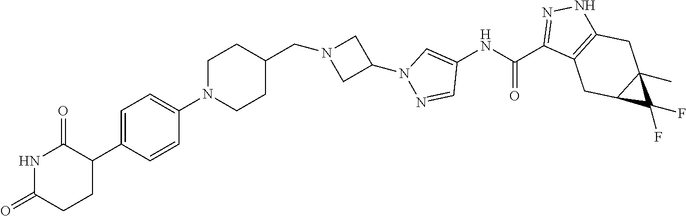 [SMILES: C[C@@]12Cc3[nH]nc(C(=O)Nc4cnn(C5CN(CC6CCN(c7ccc(C8CCC(=O)NC8=O)cc7)CC6)C5)c4)c3C[C@@H]1C2(F)F] |
| 92 | 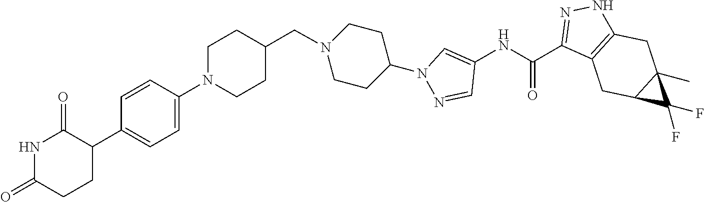 [SMILES: C[C@@]12Cc3[nH]nc(C(=O)Nc4cnn(C5CCN(CC6CCN(c7ccc(C8CCC(=O)NC8=O)cc7)CC6)CC5)c4)c3C[C@@H]1C2(F)F] |
| 93 | 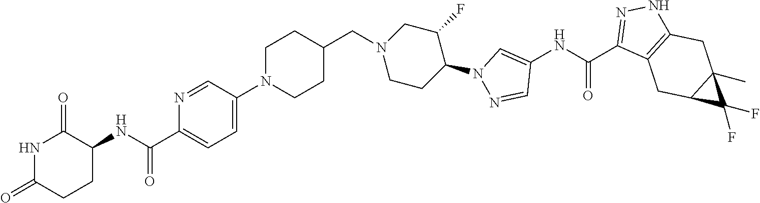 [SMILES: C[C@@]12Cc3[nH]nc(C(=O)Nc4cnn([C@H]5CCN(CC6CCN(c7ccc(C(=O)N[C@H]8CCC(=O)NC8=O)nc7)CC6)C[C@@H]5F)c4)c3C[C@@H]1C2(F)F] |
| 94 | 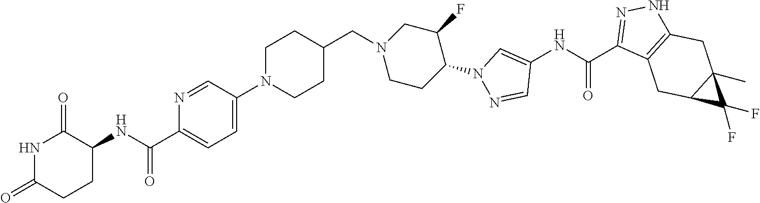 [SMILES: C[C@@]12Cc3[nH]nc(C(=O)Nc4cnn([C@@H]5CCN(CC6CCN(c7ccc(C(=O)N[C@H]8CCC(=O)NC8=O)nc7)CC6)C[C@H]5F)c4)c3C[C@@H]1C2(F)F] |
| 95 | 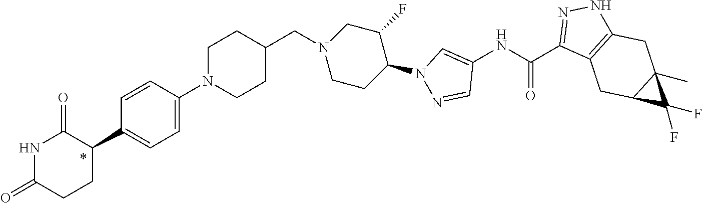 [SMILES: C[C@@]12Cc3[nH]nc(C(=O)Nc4cnn([C@H]5CCN(CC6CCN(c7ccc([C@H]8CCC(=O)NC8=O)cc7)CC6)C[C@@H]5F)c4)c3C[C@@H]1C2(F)F] |
| 96 | 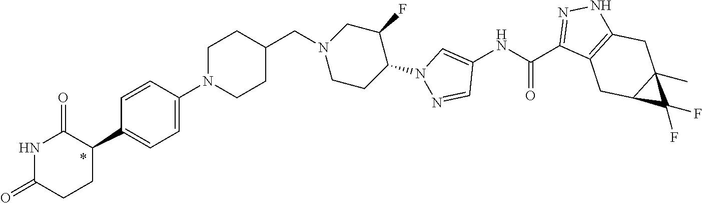 [SMILES: C[C@@]12Cc3[nH]nc(C(=O)Nc4cnn([C@@H]5CCN(CC6CCN(c7ccc([C@H]8CCC(=O)NC8=O)cc7)CC6)C[C@H]5F)c4)c3C[C@@H]1C2(F)F] |
| 97 | 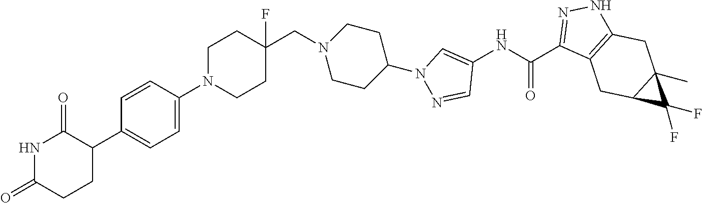 [SMILES: C[C@@]12Cc3[nH]nc(C(=O)Nc4cnn(C5CCN(CC6(F)CCN(c7ccc(C8CCC(=O)NC8=O)cc7)CC6)CC5)c4)c3C[C@@H]1C2(F)F] |
| 98 | 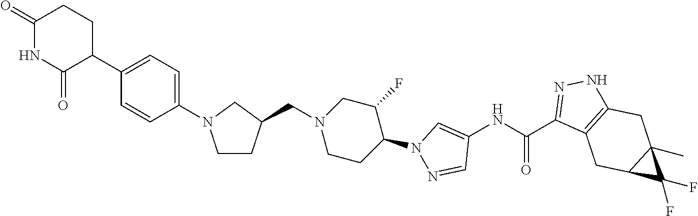 [SMILES: C[C@@]12Cc3[nH]nc(C(=O)Nc4cnn([C@H]5CCN(C[C@H]6CCN(c7ccc(C8CCC(=O)NC8=O)cc7)C6)C[C@@H]5F)c4)c3C[C@@H]1C2(F)F] |
| 99 | 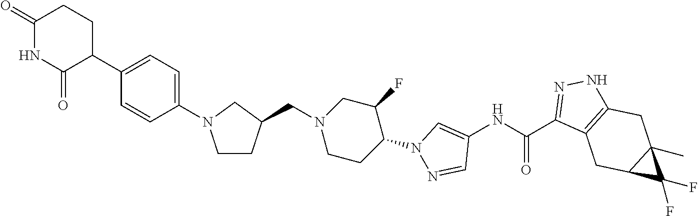 [SMILES: C[C@@]12Cc3[nH]nc(C(=O)Nc4cnn([C@@H]5CCN(C[C@H]6CCN(c7ccc(C8CCC(=O)NC8=O)cc7)C6)C[C@H]5F)c4)c3C[C@@H]1C2(F)F] |
| 100 | 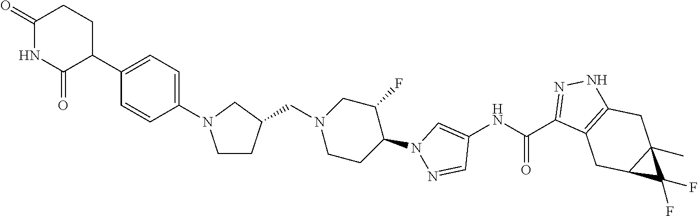 [SMILES: C[C@@]12Cc3[nH]nc(C(=O)Nc4cnn([C@H]5CCN(C[C@@H]6CCN(c7ccc(C8CCC(=O)NC8=O)cc7)C6)C[C@@H]5F)c4)c3C[C@@H]1C2(F)F] |
| 101 | 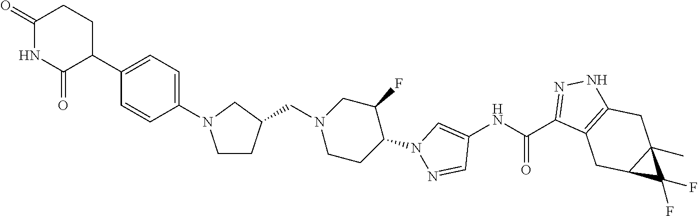 [SMILES: C[C@@]12Cc3[nH]nc(C(=O)Nc4cnn([C@@H]5CCN(C[C@@H]6CCN(c7ccc(C8CCC(=O)NC8=O)cc7)C6)C[C@H]5F)c4)c3C[C@@H]1C2(F)F] |
| 102 | 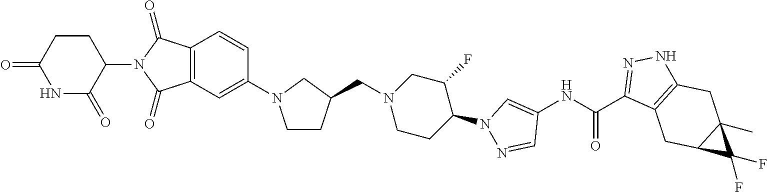 [SMILES: C[C@@]12Cc3[nH]nc(C(=O)Nc4cnn([C@H]5CCN(C[C@H]6CCN(c7ccc8c(c7)C(=O)N(C7CCC(=O)NC7=O)C8=O)C6)C[C@@H]5F)c4)c3C[C@@H]1C2(F)F] |
| 103 | 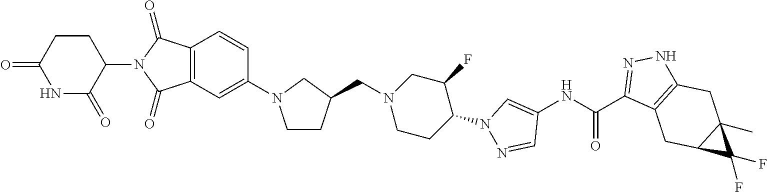 [SMILES: C[C@@]12Cc3[nH]nc(C(=O)Nc4cnn([C@@H]5CCN(C[C@H]6CCN(c7ccc8c(c7)C(=O)N(C7CCC(=O)NC7=O)C8=O)C6)C[C@H]5F)c4)c3C[C@@H]1C2(F)F] |
| 104 | 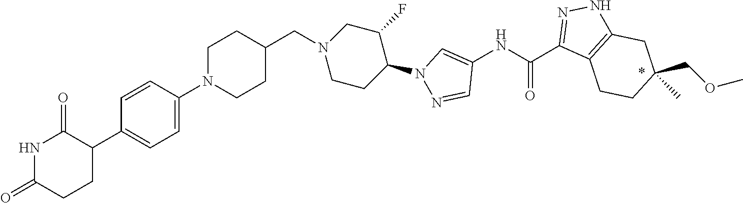 [SMILES: COC[C@@]1(C)CCc2c(C(=O)Nc3cnn([C@H]4CCN(CC5CCN(c6ccc(C7CCC(=O)NC7=O)cc6)CC5)C[C@@H]4F)c3)n[nH]c2C1] |
| 105 | 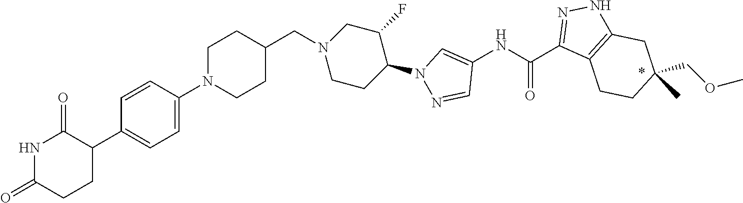 [SMILES: COC[C@]1(C)CCc2c(C(=O)Nc3cnn([C@H]4CCN(CC5CCN(c6ccc(C7CCC(=O)NC7=O)cc6)CC5)C[C@@H]4F)c3)n[nH]c2C1] |
| 106 | 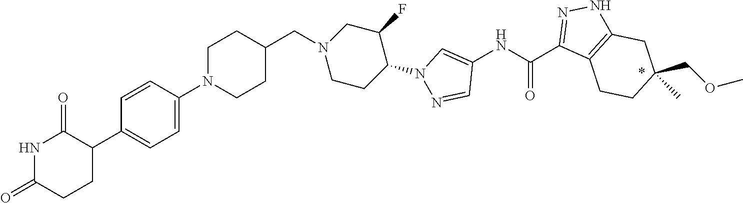 [SMILES: COC[C@@]1(C)CCc2c(C(=O)Nc3cnn([C@@H]4CCN(CC5CCN(c6ccc(C7CCC(=O)NC7=O)cc6)CC5)C[C@H]4F)c3)n[nH]c2C1] |
| 107 | 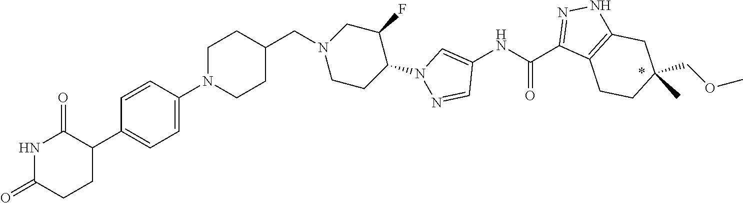 [SMILES: COC[C@]1(C)CCc2c(C(=O)Nc3cnn([C@@H]4CCN(CC5CCN(c6ccc(C7CCC(=O)NC7=O)cc6)CC5)C[C@H]4F)c3)n[nH]c2C1] |
| 108 | 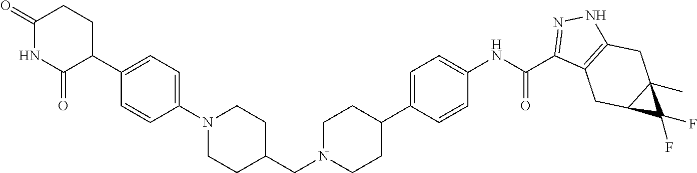 [SMILES: C[C@@]12Cc3[nH]nc(C(=O)Nc4ccc(C5CCN(CC6CCN(c7ccc(C8CCC(=O)NC8=O)cc7)CC6)CC5)cc4)c3C[C@@H]1C2(F)F] |
| 109 | 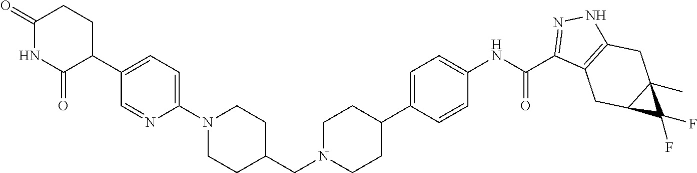 [SMILES: C[C@@]12Cc3[nH]nc(C(=O)Nc4ccc(C5CCN(CC6CCN(c7ccc(C8CCC(=O)NC8=O)cn7)CC6)CC5)cc4)c3C[C@@H]1C2(F)F] |
| 110 | 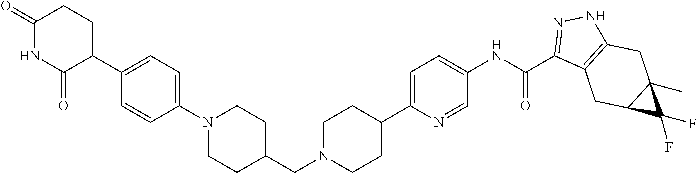 [SMILES: C[C@@]12Cc3[nH]nc(C(=O)Nc4ccc(C5CCN(CC6CCN(c7ccc(C8CCC(=O)NC8=O)cc7)CC6)CC5)nc4)c3C[C@@H]1C2(F)F] |
| 111 | 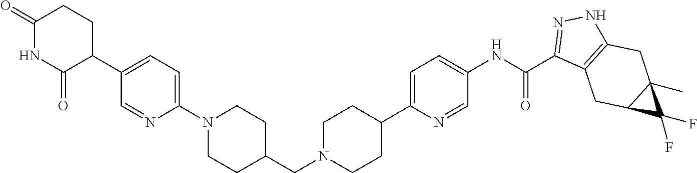 [SMILES: C[C@@]12Cc3[nH]nc(C(=O)Nc4ccc(C5CCN(CC6CCN(c7ccc(C8CCC(=O)NC8=O)cn7)CC6)CC5)nc4)c3C[C@@H]1C2(F)F] |
| 112 | 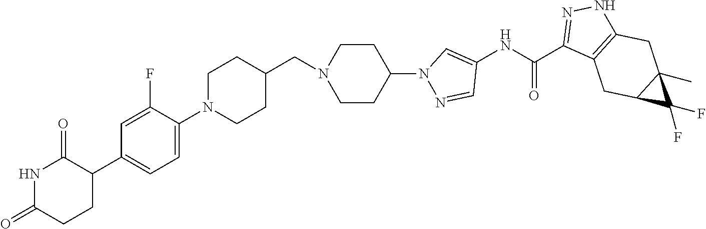 [SMILES: C[C@@]12Cc3[nH]nc(C(=O)Nc4cnn(C5CCN(CC6CCN(c7ccc(C8CCC(=O)NC8=O)cc7F)CC6)CC5)c4)c3C[C@@H]1C2(F)F] |
| 113 | 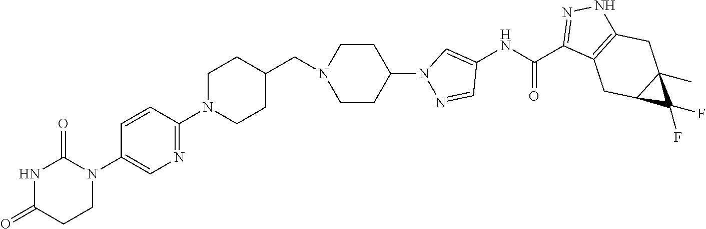 [SMILES: C[C@@]12Cc3[nH]nc(C(=O)Nc4cnn(C5CCN(CC6CCN(c7ccc(N8CCC(=O)NC8=O)cn7)CC6)CC5)c4)c3C[C@@H]1C2(F)F] |
| 114 | 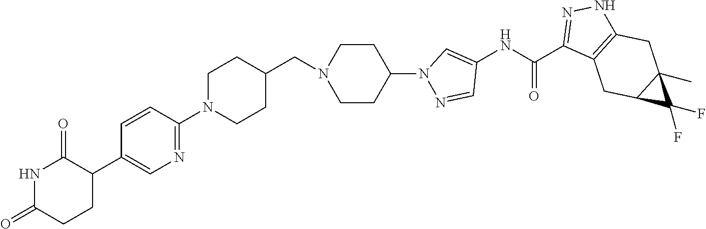 [SMILES: C[C@@]12Cc3[nH]nc(C(=O)Nc4cnn(C5CCN(CC6CCN(c7ccc(C8CCC(=O)NC8=O)cn7)CC6)CC5)c4)c3C[C@@H]1C2(F)F] |
| 115 | 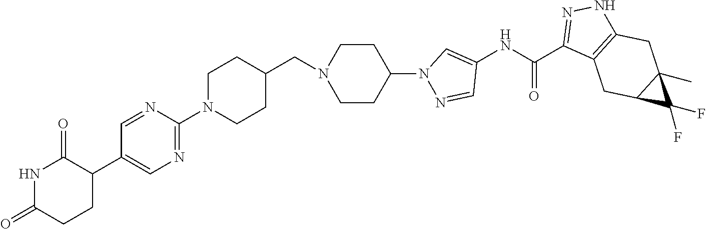 [SMILES: C[C@@]12Cc3[nH]nc(C(=O)Nc4cnn(C5CCN(CC6CCN(c7ncc(C8CCC(=O)NC8=O)cn7)CC6)CC5)c4)c3C[C@@H]1C2(F)F] |
| 116 | 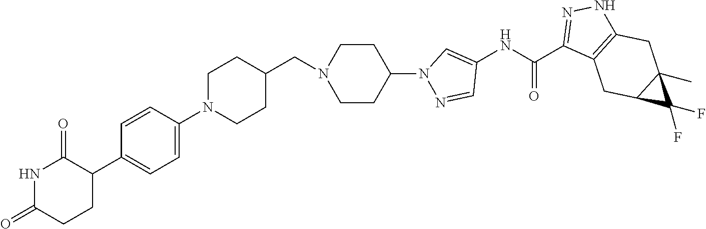 [SMILES: C[C@@]12Cc3[nH]nc(C(=O)Nc4cnn(C5CCN(CC6CCN(c7ccc(C8CCC(=O)NC8=O)cc7)CC6)CC5)c4)c3C[C@@H]1C2(F)F] |
| 117 | 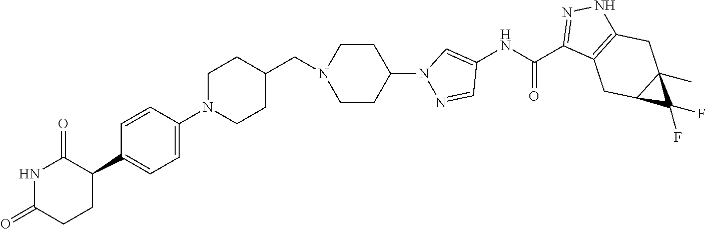 [SMILES: C[C@@]12Cc3[nH]nc(C(=O)Nc4cnn(C5CCN(CC6CCN(c7ccc([C@H]8CCC(=O)NC8=O)cc7)CC6)CC5)c4)c3C[C@@H]1C2(F)F] |
| 118 | 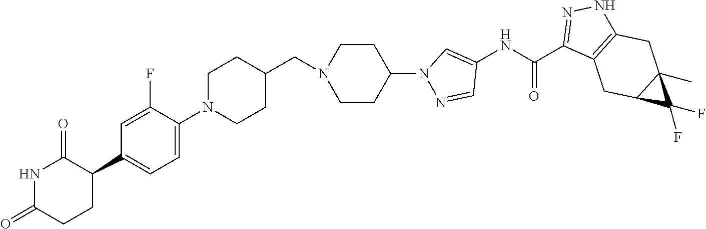 [SMILES: C[C@@]12Cc3[nH]nc(C(=O)Nc4cnn(C5CCN(CC6CCN(c7ccc([C@H]8CCC(=O)NC8=O)cc7F)CC6)CC5)c4)c3C[C@@H]1C2(F)F] |
| 119 | 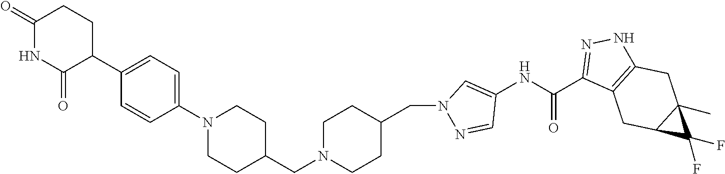 [SMILES: C[C@@]12Cc3[nH]nc(C(=O)Nc4cnn(CC5CCN(CC6CCN(c7ccc(C8CCC(=O)NC8=O)cc7)CC6)CC5)c4)c3C[C@@H]1C2(F)F] |
| 120 | 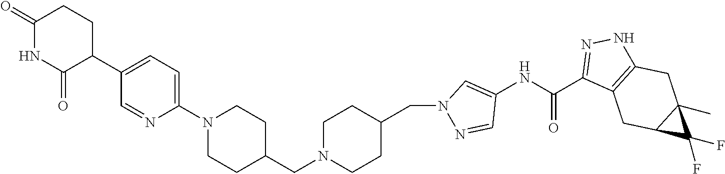 [SMILES: C[C@@]12Cc3[nH]nc(C(=O)Nc4cnn(CC5CCN(CC6CCN(c7ccc(C8CCC(=O)NC8=O)cn7)CC6)CC5)c4)c3C[C@@H]1C2(F)F] |
| 121 | 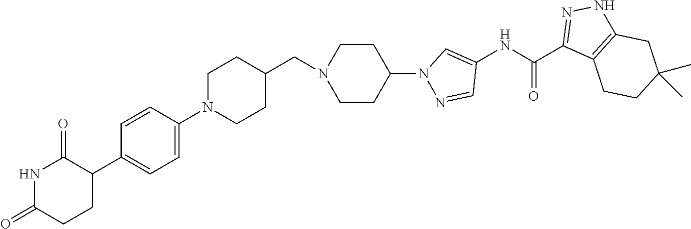 [SMILES: CC1(C)CCc2c(C(=O)Nc3cnn(C4CCN(CC5CCN(c6ccc(C7CCC(=O)NC7=O)cc6)CC5)CC4)c3)n[nH]c2C1] |
| 122 | 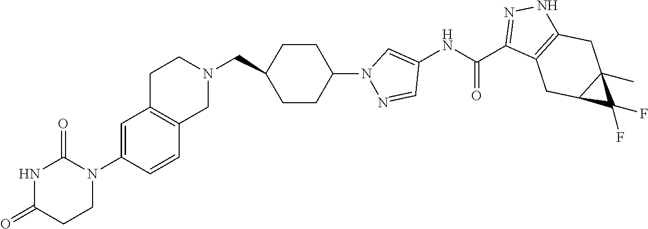 |
| 123 | 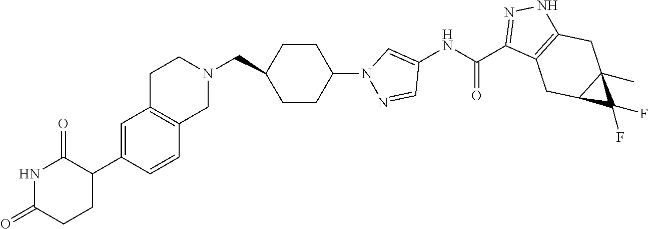 |
| 124 | 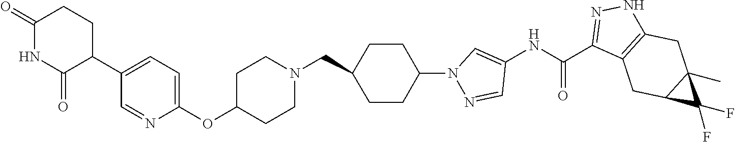 |
| 125 | 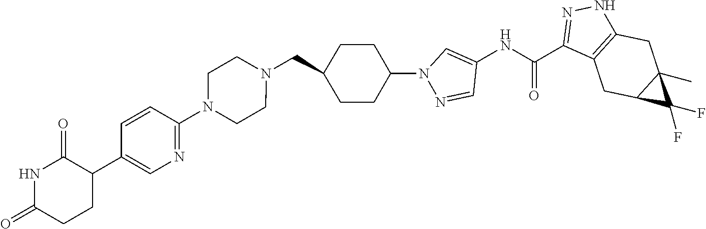 |
| 126 | 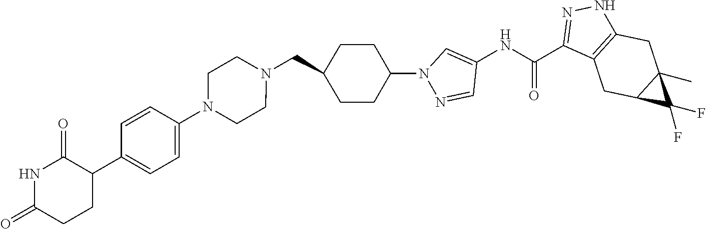 |
| 127 | 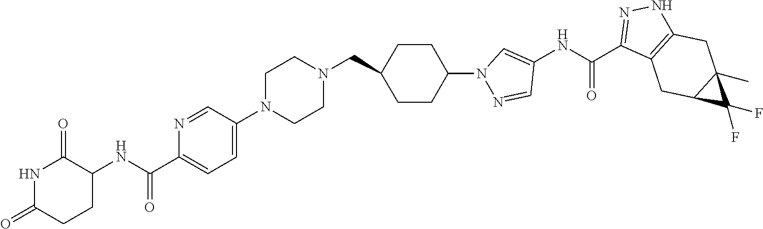 |
| 128 | 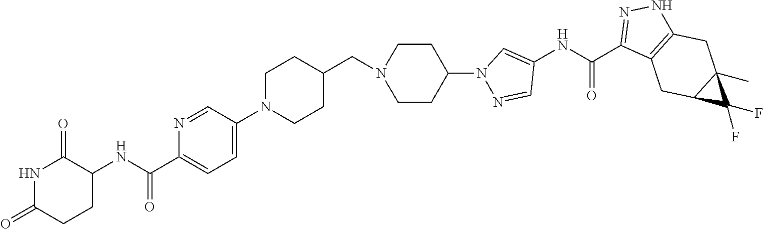 [SMILES: C[C@@]12Cc3[nH]nc(C(=O)Nc4cnn(C5CCN(CC6CCN(c7ccc(C(=O)NC8CCC(=O)NC8=O)nc7)CC6)CC5)c4)c3C[C@@H]1C2(F)F] |
| 129 | 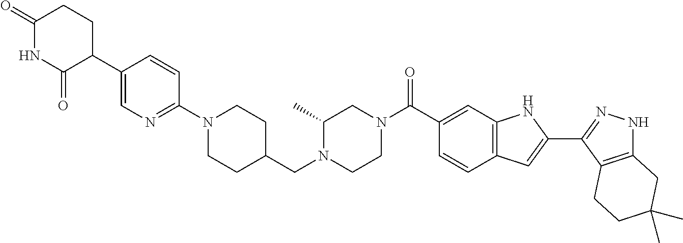 [SMILES: C[C@@H]1CN(C(=O)c2ccc3cc(-c4n[nH]c5c4CCC(C)(C)C5)[nH]c3c2)CCN1CC1CCN(c2ccc(C3CCC(=O)NC3=O)cn2)CC1] |
| 130 | 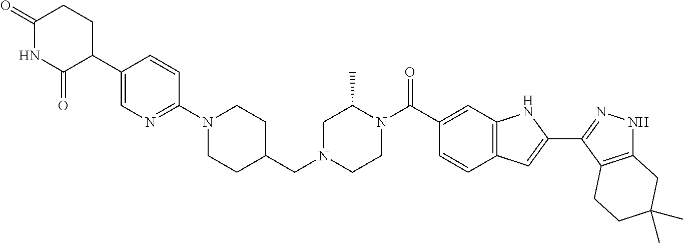 [SMILES: C[C@H]1CN(CC2CCN(c3ccc(C4CCC(=O)NC4=O)cn3)CC2)CCN1C(=O)c1ccc2cc(-c3n[nH]c4c3CCC(C)(C)C4)[nH]c2c1] |
| 131 | 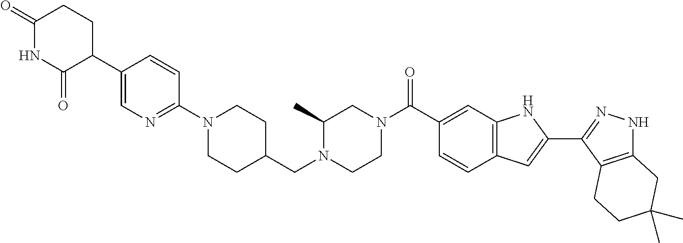 [SMILES: C[C@H]1CN(C(=O)c2ccc3cc(-c4n[nH]c5c4CCC(C)(C)C5)[nH]c3c2)CCN1CC1CCN(c2ccc(C3CCC(=O)NC3=O)cn2)CC1] |
| 132 | 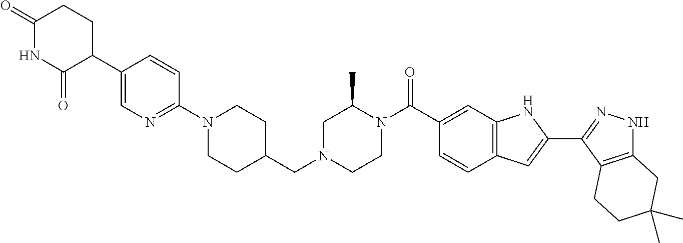 [SMILES: C[C@@H]1CN(CC2CCN(c3ccc(C4CCC(=O)NC4=O)cn3)CC2)CCN1C(=O)c1ccc2cc(-c3n[nH]c4c3CCC(C)(C)C4)[nH]c2c1] |
| 133 | 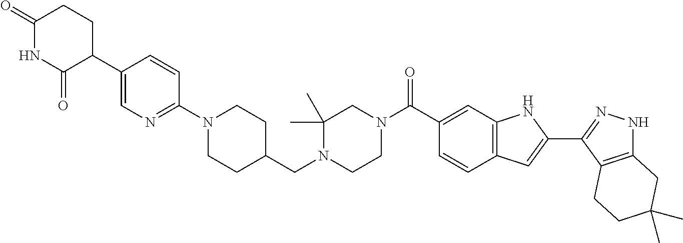 [SMILES: CC1(C)CCc2c(-c3cc4ccc(C(=O)N5CCN(CC6CCN(c7ccc(C8CCC(=O)NC8=O)cn7)CC6)C(C)(C)C5)cc4[nH]3)n[nH]c2C1] |
| 134 | 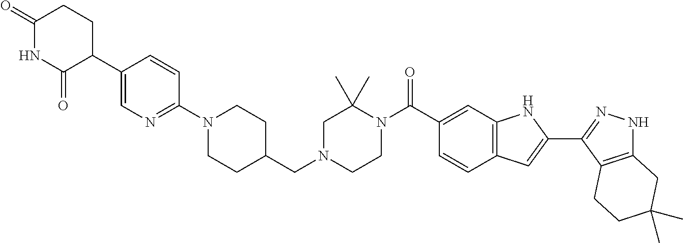 [SMILES: CC1(C)CCc2c(-c3cc4ccc(C(=O)N5CCN(CC6CCN(c7ccc(C8CCC(=O)NC8=O)cn7)CC6)CC5(C)C)cc4[nH]3)n[nH]c2C1] |
| 135 | 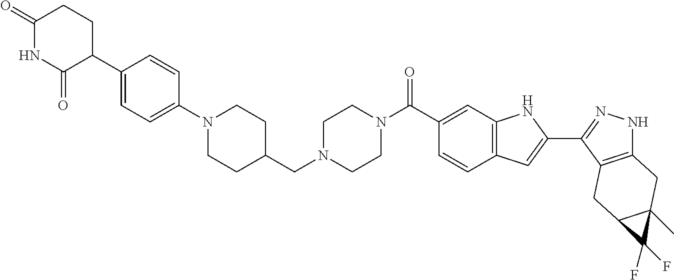 [SMILES: C[C@@]12Cc3[nH]nc(-c4cc5ccc(C(=O)N6CCN(CC7CCN(c8ccc(C9CCC(=O)NC9=O)cc8)CC7)CC6)cc5[nH]4)c3C[C@@H]1C2(F)F] |
| 136 | 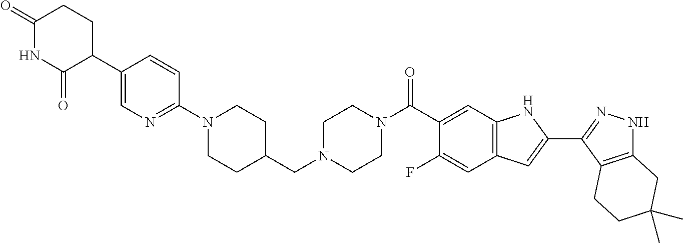 [SMILES: CC1(C)CCc2c(-c3cc4cc(F)c(C(=O)N5CCN(CC6CCN(c7ccc(C8CCC(=O)NC8=O)cn7)CC6)CC5)cc4[nH]3)n[nH]c2C1] |
| 137 | 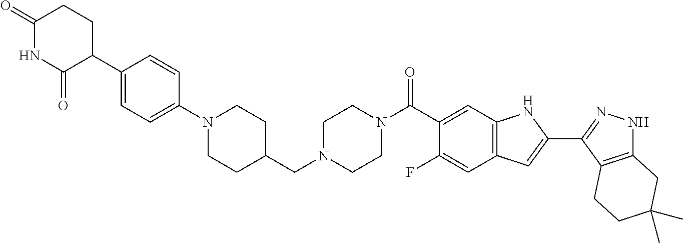 [SMILES: CC1(C)CCc2c(-c3cc4cc(F)c(C(=O)N5CCN(CC6CCN(c7ccc(C8CCC(=O)NC8=O)cc7)CC6)CC5)cc4[nH]3)n[nH]c2C1] |
| 138 | 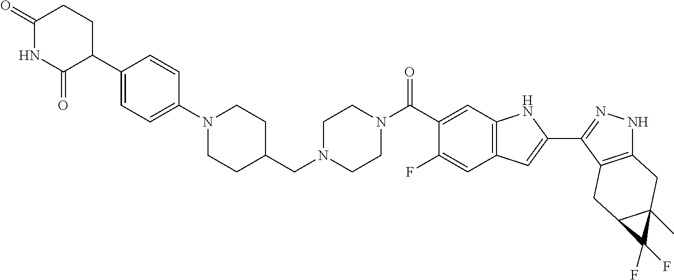 [SMILES: C[C@@]12Cc3[nH]nc(-c4cc5cc(F)c(C(=O)N6CCN(CC7CCN(c8ccc(C9CCC(=O)NC9=O)cc8)CC7)CC6)cc5[nH]4)c3C[C@@H]1C2(F)F] |
| 139 | 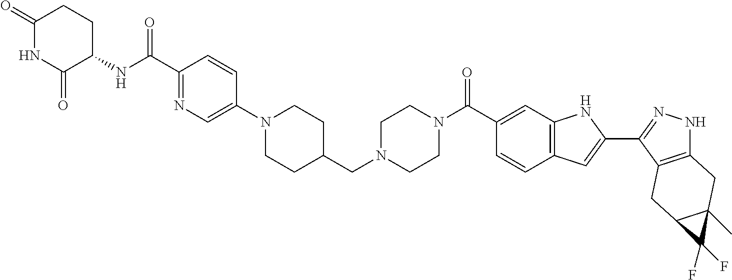 [SMILES: C[C@@]12Cc3[nH]nc(-c4cc5ccc(C(=O)N6CCN(CC7CCN(c8ccc(C(=O)N[C@H]9CCC(=O)NC9=O)nc8)CC7)CC6)cc5[nH]4)c3C[C@@H]1C2(F)F] |
| 140 | 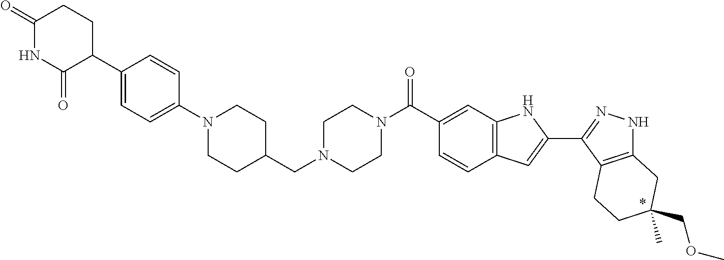 [SMILES: COC[C@@]1(C)CCc2c(-c3cc4ccc(C(=O)N5CCN(CC6CCN(c7ccc(C8CCC(=O)NC8=O)cc7)CC6)CC5)cc4[nH]3)n[nH]c2C1] |
| 141 | 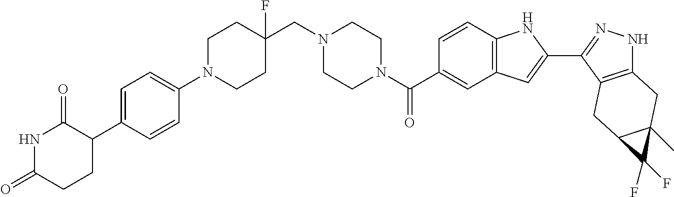 [SMILES: C[C@@]12Cc3[nH]nc(-c4cc5cc(C(=O)N6CCN(CC7(F)CCN(c8ccc(C9CCC(=O)NC9=O)cc8)CC7)CC6)ccc5[nH]4)c3C[C@@H]1C2(F)F] |
| 142 | 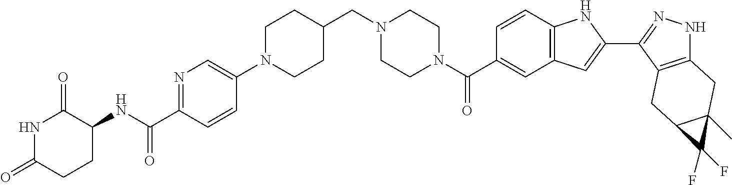 [SMILES: C[C@@]12Cc3[nH]nc(-c4cc5cc(C(=O)N6CCN(CC7CCN(c8ccc(C(=O)N[C@H]9CCC(=O)NC9=O)nc8)CC7)CC6)ccc5[nH]4)c3C[C@@H]1C2(F)F] |
| 143 | 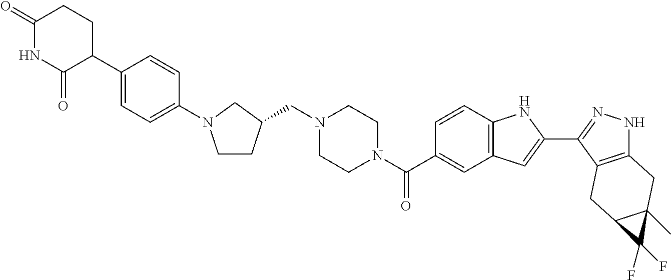 [SMILES: C[C@@]12Cc3[nH]nc(-c4cc5cc(C(=O)N6CCN(C[C@@H]7CCN(c8ccc(C9CCC(=O)NC9=O)cc8)C7)CC6)ccc5[nH]4)c3C[C@@H]1C2(F)F] |
| 144 | 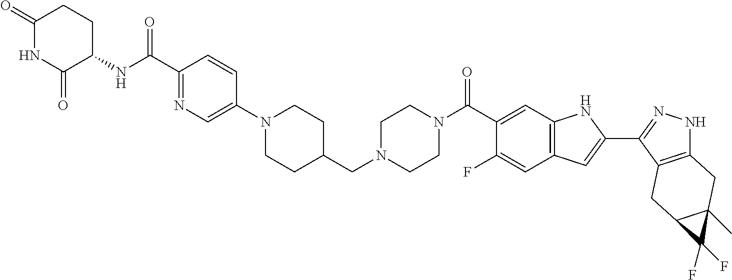 [SMILES: C[C@@]12Cc3[nH]nc(-c4cc5cc(F)c(C(=O)N6CCN(CC7CCN(c8ccc(C(=O)N[C@H]9CCC(=O)NC9=O)nc8)CC7)CC6)cc5[nH]4)c3C[C@@H]1C2(F)F] |
| 145 | 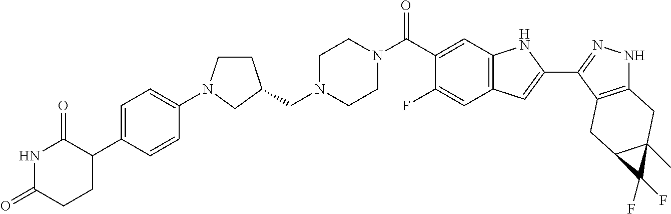 [SMILES: C[C@@]12Cc3[nH]nc(-c4cc5cc(F)c(C(=O)N6CCN(C[C@H]7CCN(c8ccc(C9CCC(=O)NC9=O)cc8)C7)CC6)cc5[nH]4)c3C[C@@H]1C2(F)F] |
| 146 | 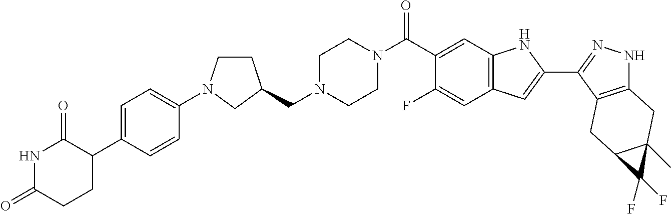 [SMILES: C[C@@]12Cc3[nH]nc(-c4cc5cc(F)c(C(=O)N6CCN(C[C@@H]7CCN(c8ccc(C9CCC(=O)NC9=O)cc8)C7)CC6)cc5[nH]4)c3C[C@@H]1C2(F)F] |
| 147 | 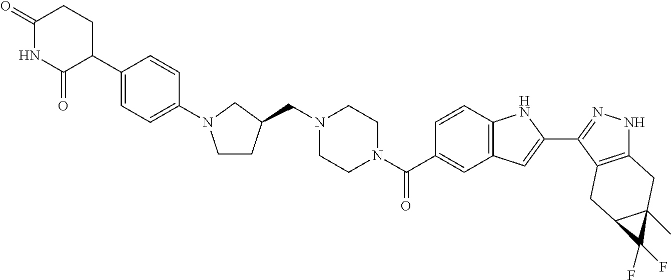 [SMILES: C[C@@]12Cc3[nH]nc(-c4cc5cc(C(=O)N6CCN(C[C@H]7CCN(c8ccc(C9CCC(=O)NC9=O)cc8)C7)CC6)ccc5[nH]4)c3C[C@@H]1C2(F)F] |
| 148 | 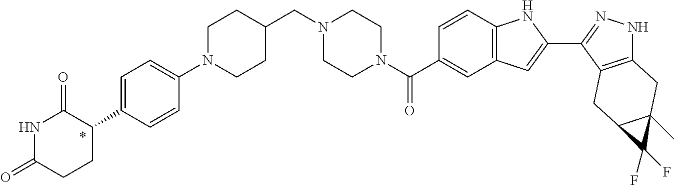 [SMILES: C[C@@]12Cc3[nH]nc(-c4cc5cc(C(=O)N6CCN(CC7CCN(c8ccc([C@@H]9CCC(=O)NC9=O)cc8)CC7)CC6)ccc5[nH]4)c3C[C@@H]1C2(F)F] |
| 149 | 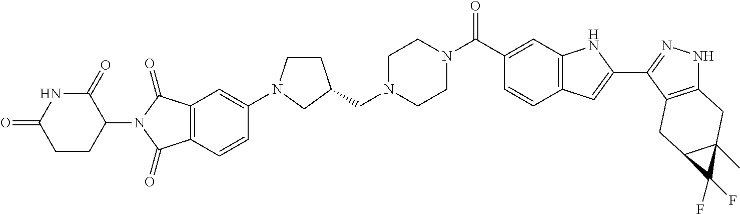 [SMILES: C[C@@]12Cc3[nH]nc(-c4cc5ccc(C(=O)N6CCN(C[C@H]7CCN(c8ccc9c(c8)C(=O)N(C8CCC(=O)NC8=O)C9=O)C7)CC6)cc5[nH]4)c3C[C@@H]1C2(F)F] |
| 150 | 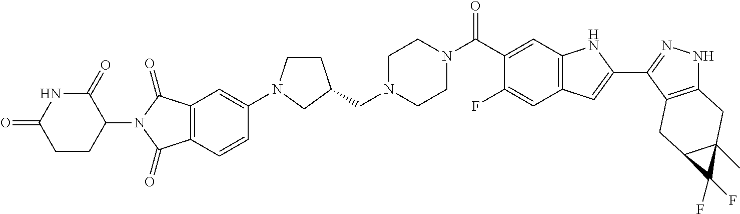 [SMILES: C[C@@]12Cc3[nH]nc(-c4cc5cc(F)c(C(=O)N6CCN(C[C@H]7CCN(c8ccc9c(c8)C(=O)N(C8CCC(=O)NC8=O)C9=O)C7)CC6)cc5[nH]4)c3C[C@@H]1C2(F)F] |
| 151 | 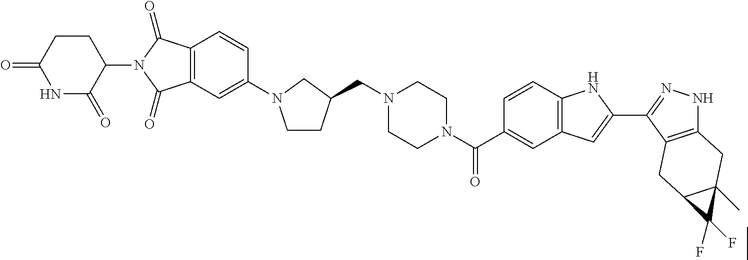 |
| 152 | 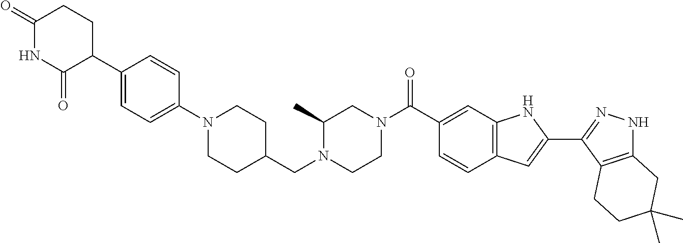 [SMILES: C[C@H]1CN(C(=O)c2ccc3cc(-c4n[nH]c5c4CCC(C)(C)C5)[nH]c3c2)CCN1CC1CCN(c2ccc(C3CCC(=O)NC3=O)cc2)CC1] |
| 153 | 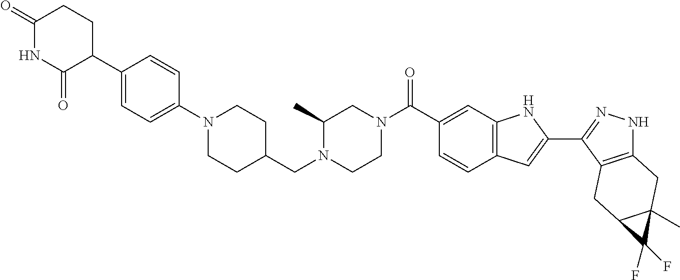 [SMILES: C[C@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)cc2)CC1] |
| 154 | 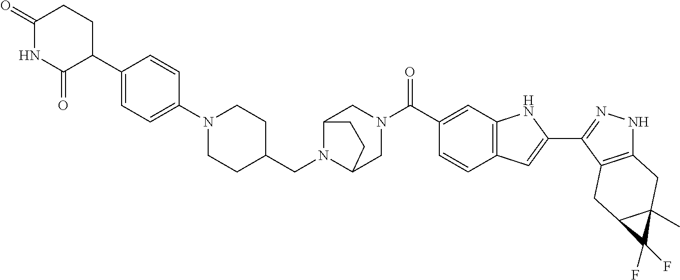 [SMILES: C[C@@]12Cc3[nH]nc(-c4cc5ccc(C(=O)N6CC7CCC(C6)N7CC6CCN(c7ccc(C8CCC(=O)NC8=O)cc7)CC6)cc5[nH]4)c3C[C@@H]1C2(F)F] |
| 155 | 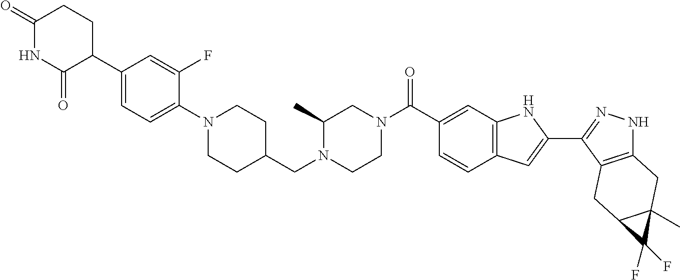 [SMILES: C[C@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)cc2F)CC1] |
| 156 | 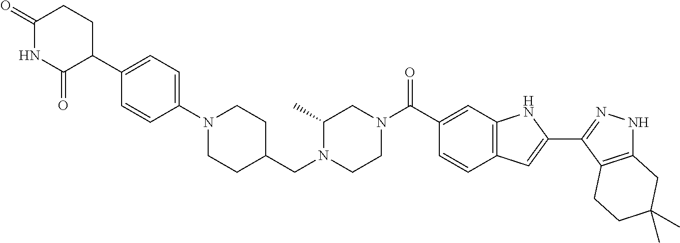 [SMILES: C[C@@H]1CN(C(=O)c2ccc3cc(-c4n[nH]c5c4CCC(C)(C)C5)[nH]c3c2)CCN1CC1CCN(c2ccc(C3CCC(=O)NC3=O)cc2)CC1] |
| 157 | 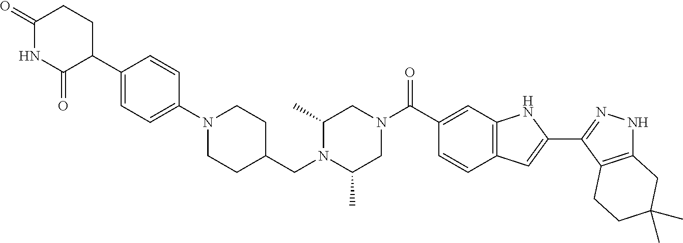 [SMILES: C[C@@H]1CN(C(=O)c2ccc3cc(-c4n[nH]c5c4CCC(C)(C)C5)[nH]c3c2)C[C@H](C)N1CC1CCN(c2ccc(C3CCC(=O)NC3=O)cc2)CC1] |
| 158 | 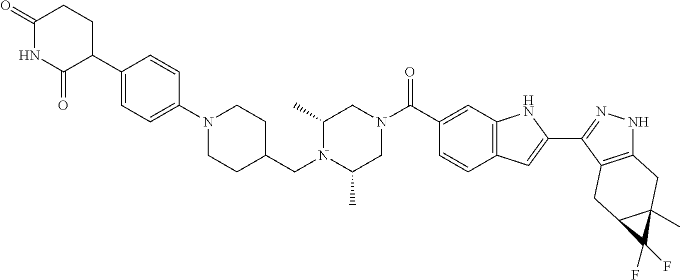 [SMILES: C[C@@H]1CN(C(=O)c2ccc3cc(-c4n[nH]c5c4C[C@@H]4C(F)(F)[C@]4(C)C5)[nH]c3c2)C[C@H](C)N1CC1CCN(c2ccc(C3CCC(=O)NC3=O)cc2)CC1] |
| 159 | 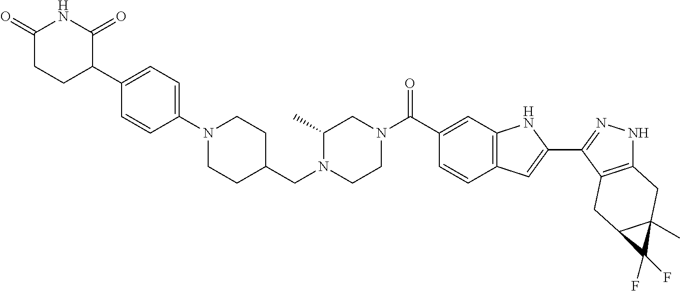 [SMILES: C[C@@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)cc2)CC1] |
| 160 | 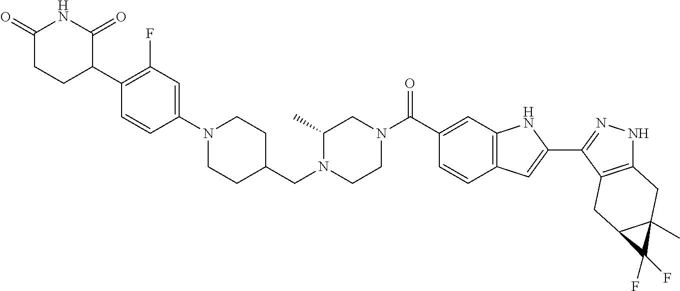 [SMILES: C[C@@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)c(F)c2)CC1] |
| 161 | 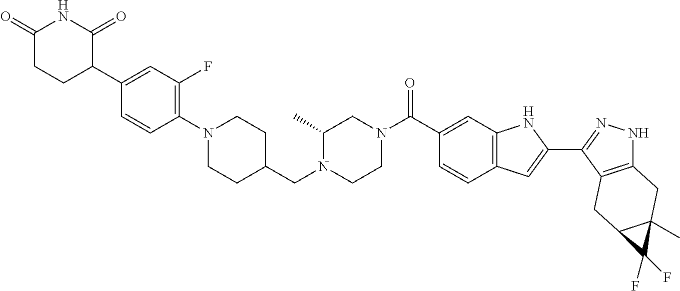 [SMILES: C[C@@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)cc2F)CC1] |
| 162 | 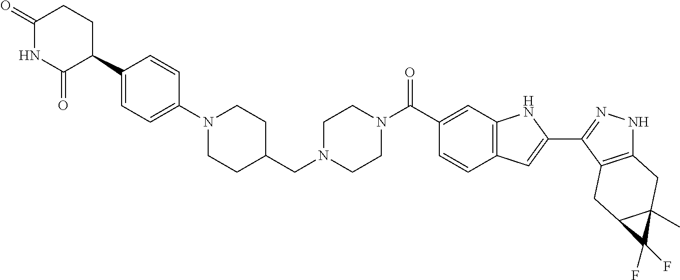 [SMILES: C[C@@]12Cc3[nH]nc(-c4cc5ccc(C(=O)N6CCN(CC7CCN(c8ccc([C@@H]9CCC(=O)NC9=O)cc8)CC7)CC6)cc5[nH]4)c3C[C@@H]1C2(F)F] |
| 163 | 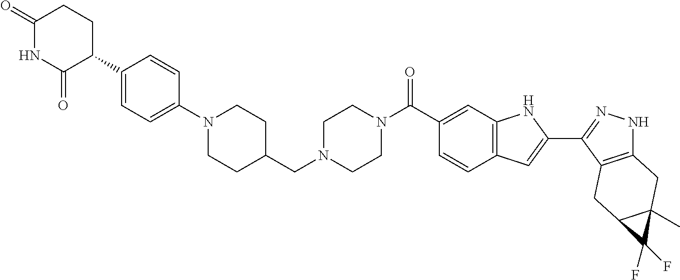 [SMILES: C[C@@]12Cc3[nH]nc(-c4cc5ccc(C(=O)N6CCN(CC7CCN(c8ccc([C@H]9CCC(=O)NC9=O)cc8)CC7)CC6)cc5[nH]4)c3C[C@@H]1C2(F)F] |
| 164 | 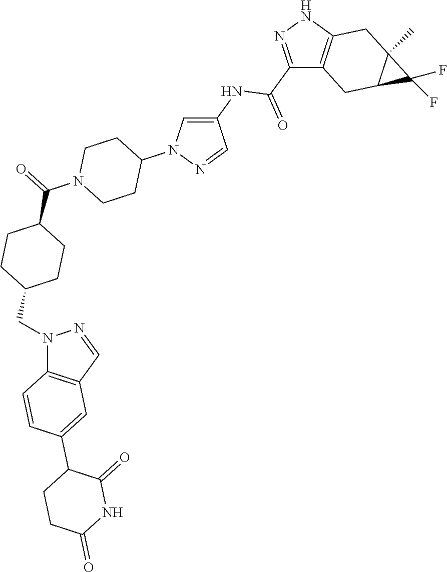 [SMILES: C[C@@]12Cc3[nH]nc(C(=O)Nc4cnn(C5CCN(C(=O)[C@H]6CC[C@H](Cn7ncc8cc(C9CCC(=O)NC9=O)ccc87)CC6)CC5)c4)c3C[C@@H]1C2(F)F] |
| 165 | 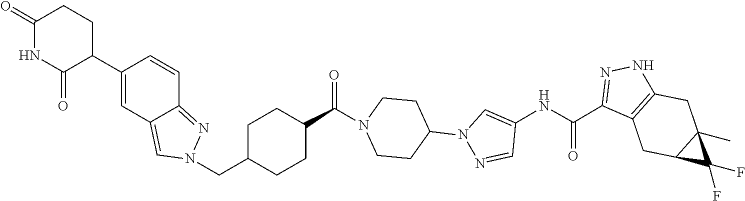 |
| 166 | 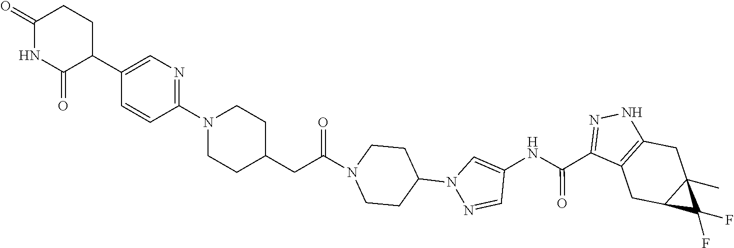 [SMILES: C[C@@]12Cc3[nH]nc(C(=O)Nc4cnn(C5CCN(C(=O)CC6CCN(c7ccc(C8CCC(=O)NC8=O)cn7)CC6)CC5)c4)c3C[C@@H]1C2(F)F] |
| 167 | 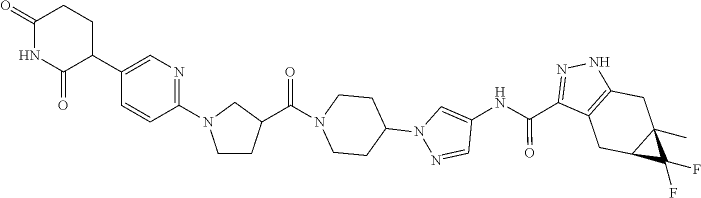 [SMILES: C[C@@]12Cc3[nH]nc(C(=O)Nc4cnn(C5CCN(C(=O)C6CCN(c7ccc(C8CCC(=O)NC8=O)cn7)C6)CC5)c4)c3C[C@@H]1C2(F)F] |
| 168 | 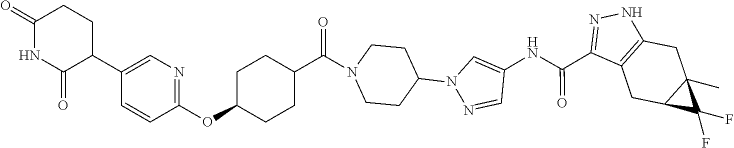 |
| 169 | 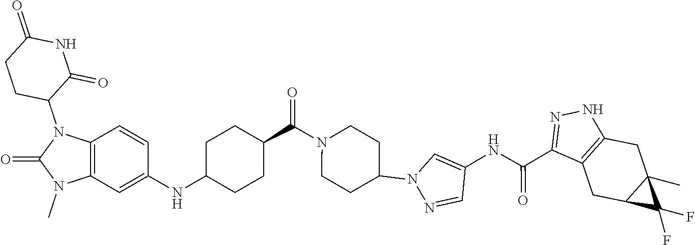 |
| 170 | 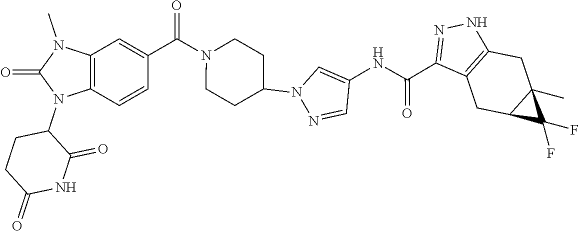 [SMILES: Cn1c(=O)n(C2CCC(=O)NC2=O)c2ccc(C(=O)N3CCC(n4cc(NC(=O)c5n[nH]c6c5C[C@@H]5C(F)(F)[C@]5(C)C6)cn4)CC3)cc21] |
| 171 | 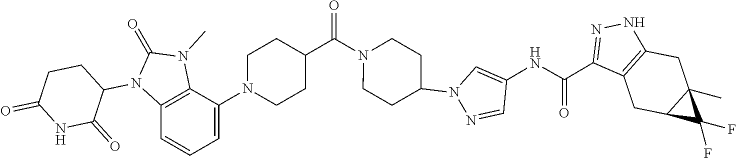 [SMILES: Cn1c(=O)n(C2CCC(=O)NC2=O)c2cccc(N3CCC(C(=O)N4CCC(n5cc(NC(=O)c6n[nH]c7c6C[C@@H]6C(F)(F)[C@]6(C)C7)cn5)CC4)CC3)c21] |
| 172 | 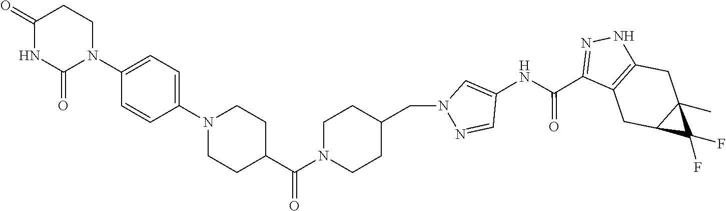 [SMILES: C[C@@]12Cc3[nH]nc(C(=O)Nc4cnn(CC5CCN(C(=O)C6CCN(c7ccc(N8CCC(=O)NC8=O)cc7)CC6)CC5)c4)c3C[C@@H]1C2(F)F] |
| 173 | 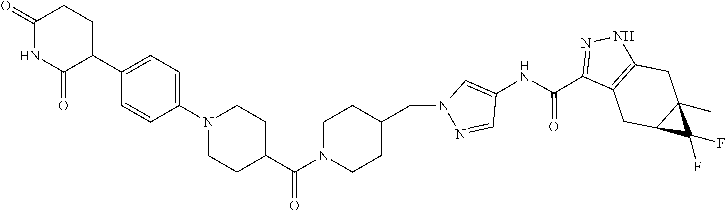 [SMILES: C[C@@]12Cc3[nH]nc(C(=O)Nc4cnn(CC5CCN(C(=O)C6CCN(c7ccc(C8CCC(=O)NC8=O)cc7)CC6)CC5)c4)c3C[C@@H]1C2(F)F] |
| 174 | 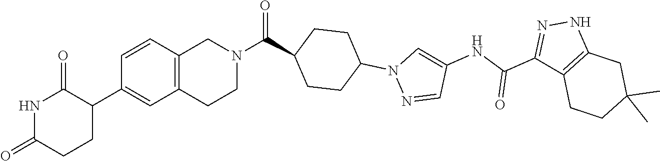 |
| 175 | 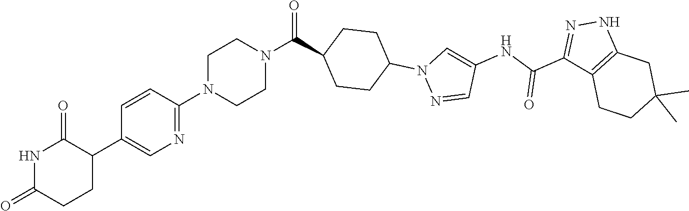 |
| 176 | 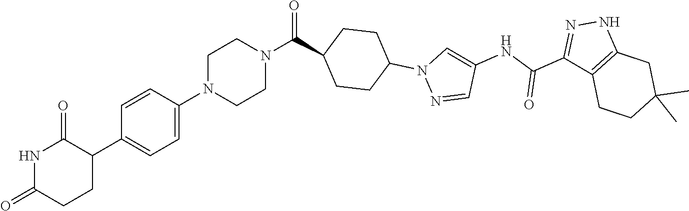 |
| 177 | 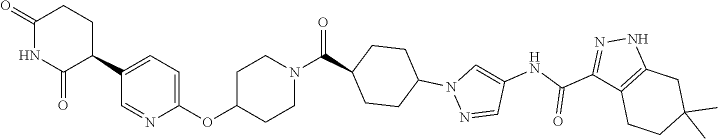 |
| 178 | 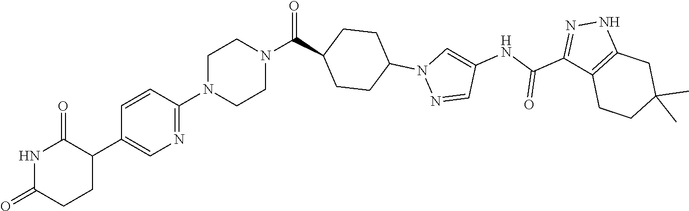 |
| 179 | 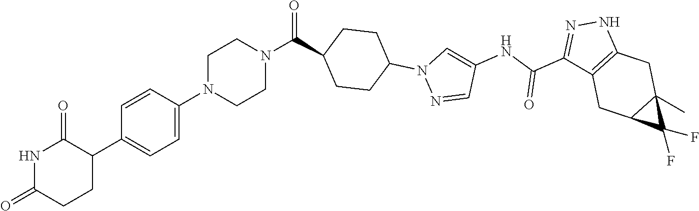 |
| 180 | 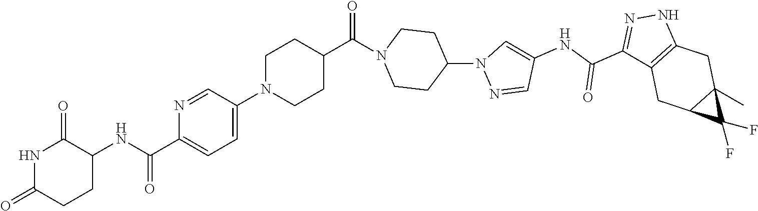 [SMILES: C[C@@]12Cc3[nH]nc(C(=O)Nc4cnn(C5CCN(C(=O)C6CCN(c7ccc(C(=O)NC8CCC(=O)NC8=O)nc7)CC6)CC5)c4)c3C[C@@H]1C2(F)F] |
| 181 | 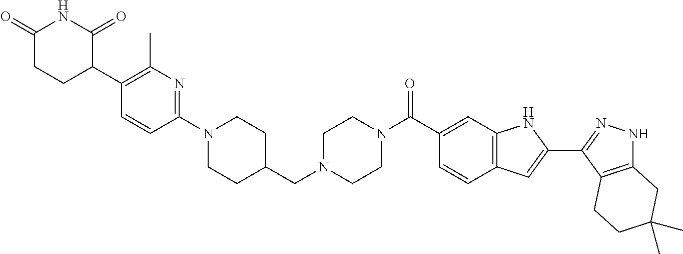 [SMILES: Cc1nc(N2CCC(CN3CCN(C(=O)c4ccc5cc(-c6n[nH]c7c6CCC(C)(C)C7)[nH]c5c4)CC3)CC2)ccc1C1CCC(=O)NC1=O] |
| 182 | 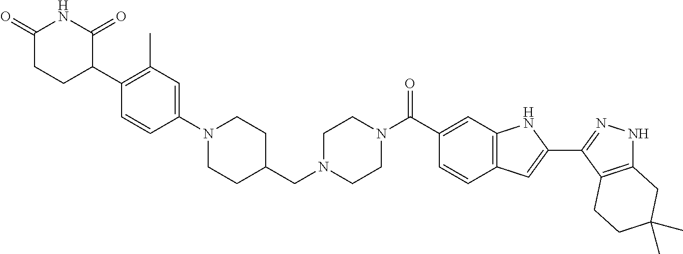 [SMILES: Cc1cc(N2CCC(CN3CCN(C(=O)c4ccc5cc(-c6n[nH]c7c6CCC(C)(C)C7)[nH]c5c4)CC3)CC2)ccc1C1CCC(=O)NC1=O] |
| 183 | 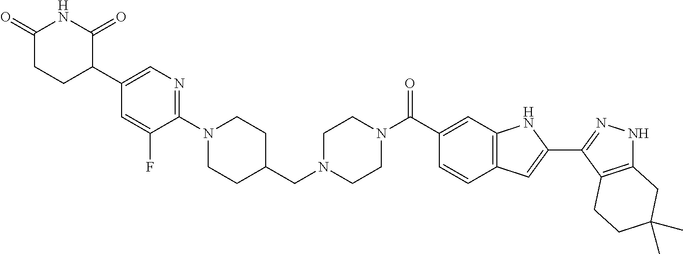 [SMILES: CC1(C)CCc2c(-c3cc4ccc(C(=O)N5CCN(CC6CCN(c7ncc(C8CCC(=O)NC8=O)cc7F)CC6)CC5)cc4[nH]3)n[nH]c2C1] |
| 184 | 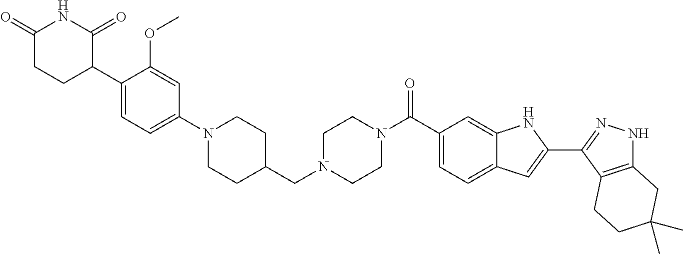 [SMILES: COc1cc(N2CCC(CN3CCN(C(=O)c4ccc5cc(-c6n[nH]c7c6CCC(C)(C)C7)[nH]c5c4)CC3)CC2)ccc1C1CCC(=O)NC1=O] |
| 185 | 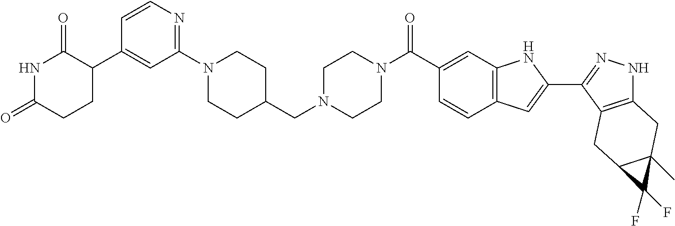 [SMILES: C[C@@]12Cc3[nH]nc(-c4cc5ccc(C(=O)N6CCN(CC7CCN(c8cc(C9CCC(=O)NC9=O)ccn8)CC7)CC6)cc5[nH]4)c3C[C@@H]1C2(F)F] |
| 186 | 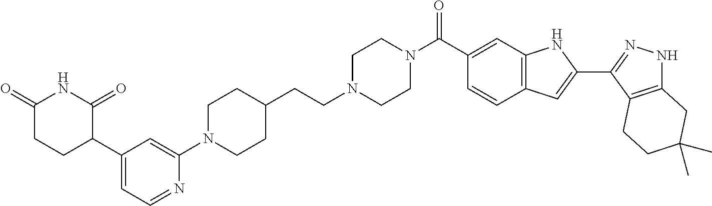 [SMILES: CC1(C)CCc2c(-c3cc4ccc(C(=O)N5CCN(CCC6CCN(c7cc(C8CCC(=O)NC8=O)ccn7)CC6)CC5)cc4[nH]3)n[nH]c2C1] |
| 187 | 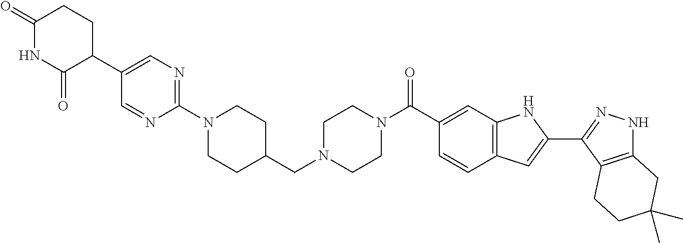 [SMILES: CC1(C)CCc2c(-c3cc4ccc(C(=O)N5CCN(CC6CCN(c7ncc(C8CCC(=O)NC8=O)cn7)CC6)CC5)cc4[nH]3)n[nH]c2C1] |
| 188 | 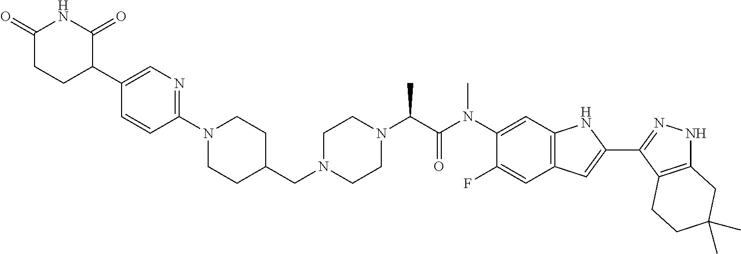 [SMILES: C[C@@H](C(=O)N(C)c1cc2[nH]c(-c3n[nH]c4c3CCC(C)(C)C4)cc2cc1F)N1CCN(CC2CCN(c3ccc(C4CCC(=O)NC4=O)cn3)CC2)CC1] |
| 189 | 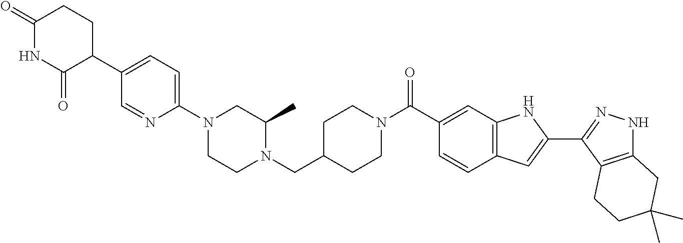 [SMILES: C[C@@H]1CN(c2ccc(C3CCC(=O)NC3=O)cn2)CCN1CC1CCN(C(=O)c2ccc3cc(-c4n[nH]c5c4CCC(C)(C)C5)[nH]c3c2)CC1] |
| 190 | 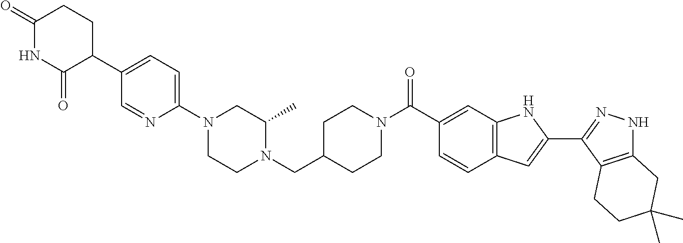 [SMILES: C[C@H]1CN(c2ccc(C3CCC(=O)NC3=O)cn2)CCN1CC1CCN(C(=O)c2ccc3cc(-c4n[nH]c5c4CCC(C)(C)C5)[nH]c3c2)CC1] |
| 191 | 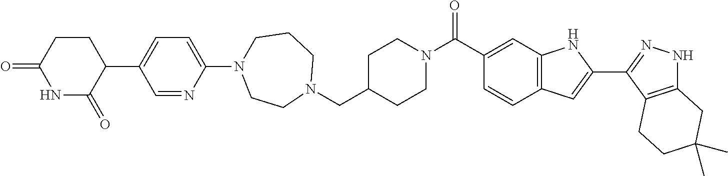 [SMILES: CC1(C)CCc2c(-c3cc4ccc(C(=O)N5CCC(CN6CCCN(c7ccc(C8CCC(=O)NC8=O)cn7)CC6)CC5)cc4[nH]3)n[nH]c2C1] |
| 192 | 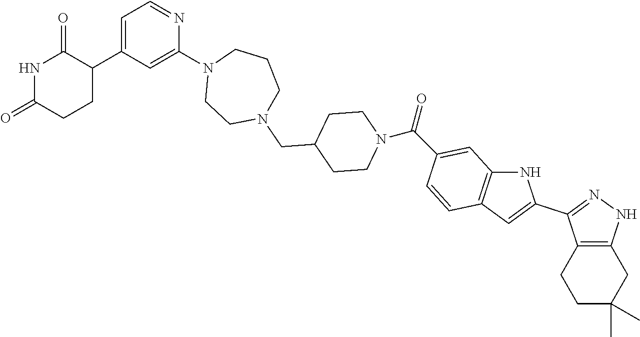 [SMILES: CC1(C)CCc2c(-c3cc4ccc(C(=O)N5CCC(CN6CCCN(c7cc(C8CCC(=O)NC8=O)ccn7)CC6)CC5)cc4[nH]3)n[nH]c2C1] |
| 193 | 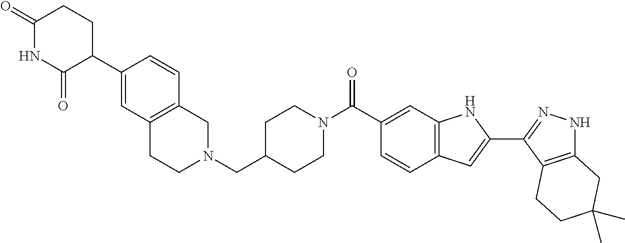 [SMILES: CC1(C)CCc2c(-c3cc4ccc(C(=O)N5CCC(CN6CCc7cc(C8CCC(=O)NC8=O)ccc7C6)CC5)cc4[nH]3)n[nH]c2C1] |
| 194 | 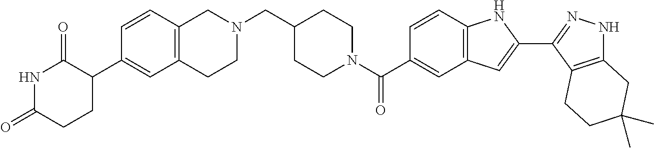 [SMILES: CC1(C)CCc2c(-c3cc4cc(C(=O)N5CCC(CN6CCc7cc(C8CCC(=O)NC8=O)ccc7C6)CC5)ccc4[nH]3)n[nH]c2C1] |
| 195 | 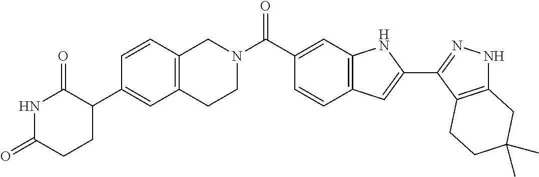 [SMILES: CC1(C)CCc2c(-c3cc4ccc(C(=O)N5CCc6cc(C7CCC(=O)NC7=O)ccc6C5)cc4[nH]3)n[nH]c2C1] |
| 196 | 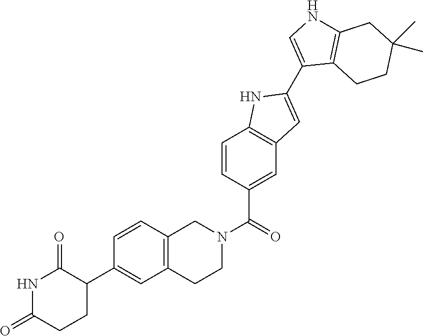 [SMILES: CC1(C)CCc2c(-c3cc4cc(C(=O)N5CCc6cc(C7CCC(=O)NC7=O)ccc6C5)ccc4[nH]3)c[nH]c2C1] |
| 197 | 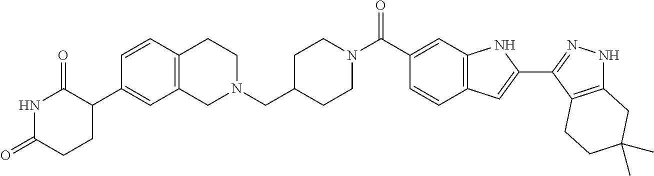 [SMILES: CC1(C)CCc2c(-c3cc4ccc(C(=O)N5CCC(CN6CCc7ccc(C8CCC(=O)NC8=O)cc7C6)CC5)cc4[nH]3)n[nH]c2C1] |
| 198 | 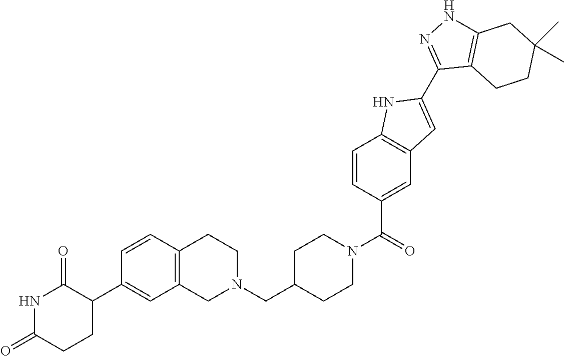 [SMILES: CC1(C)CCc2c(-c3cc4cc(C(=O)N5CCC(CN6CCc7ccc(C8CCC(=O)NC8=O)cc7C6)CC5)ccc4[nH]3)n[nH]c2C1] |
| 199 | 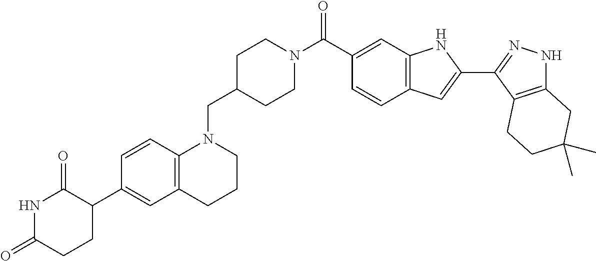 [SMILES: CC1(C)CCc2c(-c3cc4ccc(C(=O)N5CCC(CN6CCCc7cc(C8CCC(=O)NC8=O)ccc76)CC5)cc4[nH]3)n[nH]c2C1] |
| 200 | 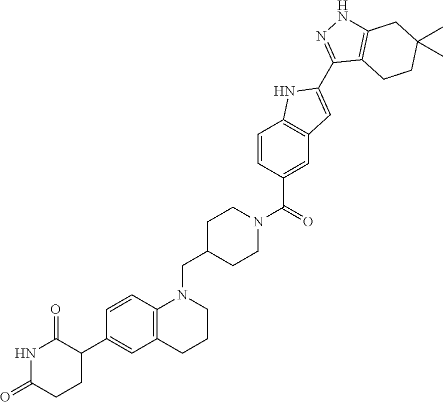 [SMILES: CC1(C)CCc2c(-c3cc4cc(C(=O)N5CCC(CN6CCCc7cc(C8CCC(=O)NC8=O)ccc76)CC5)ccc4[nH]3)n[nH]c2C1] |
| 201 | 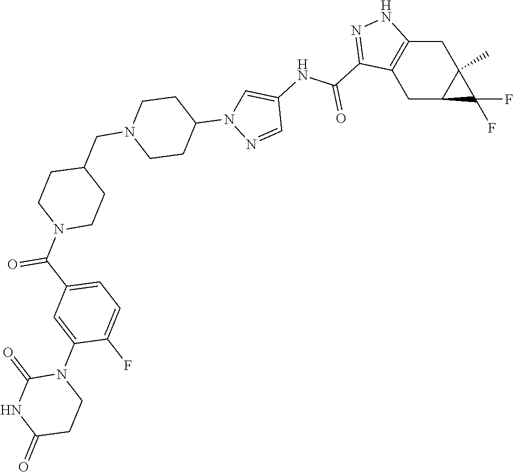 [SMILES: C[C@@]12Cc3[nH]nc(C(=O)Nc4cnn(C5CCN(CC6CCN(C(=O)c7ccc(F)c(N8CCC(=O)NC8=O)c7)CC6)CC5)c4)c3C[C@@H]1C2(F)F] |
| 202 | 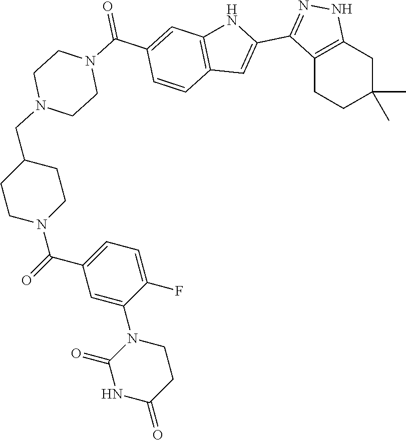 [SMILES: CC1(C)CCc2c(-c3cc4ccc(C(=O)N5CCN(CC6CCN(C(=O)c7ccc(F)c(N8CCC(=O)NC8=O)c7)CC6)CC5)cc4[nH]3)n[nH]c2C1] |
| 203 | 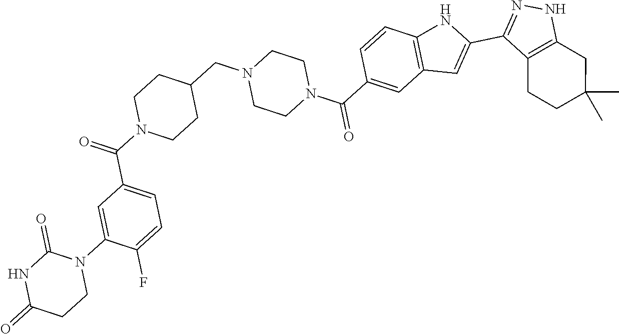 [SMILES: CC1(C)CCc2c(-c3cc4cc(C(=O)N5CCN(CC6CCN(C(=O)c7ccc(F)c(N8CCC(=O)NC8=O)c7)CC6)CC5)ccc4[nH]3)n[nH]c2C1] |
| 204 | 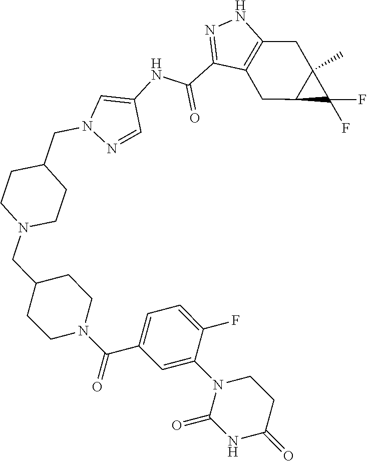 [SMILES: C[C@@]12Cc3[nH]nc(C(=O)Nc4cnn(CC5CCN(CC6CCN(C(=O)c7ccc(F)c(N8CCC(=O)NC8=O)c7)CC6)CC5)c4)c3C[C@@H]1C2(F)F] |
| 205 | 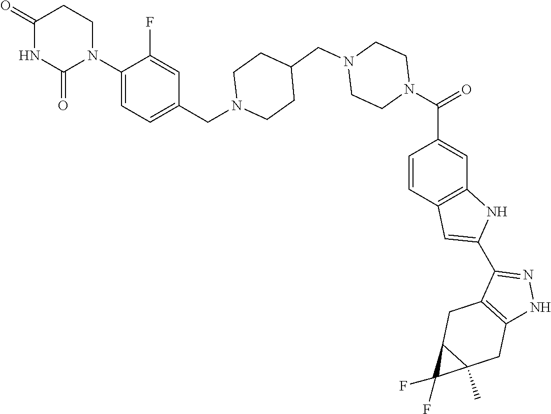 [SMILES: C[C@@]12Cc3[nH]nc(-c4cc5ccc(C(=O)N6CCN(CC7CCN(Cc8ccc(N9CCC(=O)NC9=O)c(F)c8)CC7)CC6)cc5[nH]4)c3C[C@@H]1C2(F)F] |
| 206 | 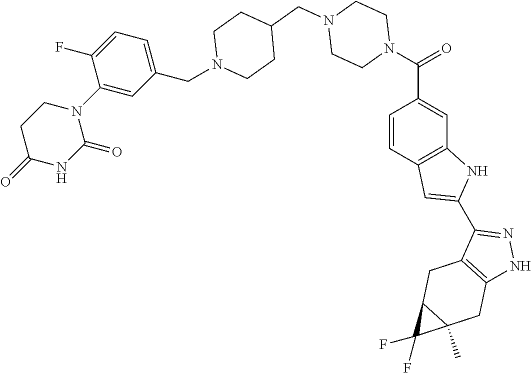 [SMILES: C[C@@]12Cc3[nH]nc(-c4cc5ccc(C(=O)N6CCN(CC7CCN(Cc8ccc(F)c(N9CCC(=O)NC9=O)c8)CC7)CC6)cc5[nH]4)c3C[C@@H]1C2(F)F] |
| 207 | 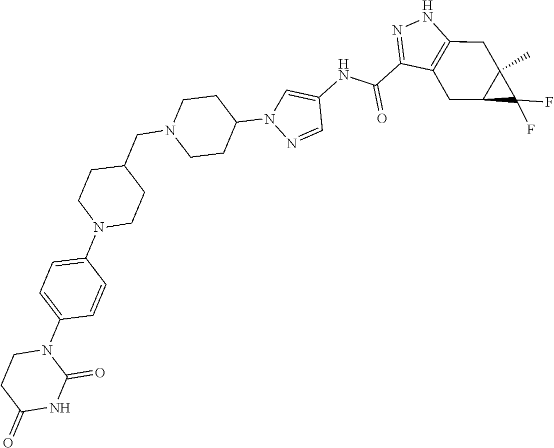 [SMILES: C[C@@]12Cc3[nH]nc(C(=O)Nc4cnn(C5CCN(CC6CCN(c7ccc(N8CCC(=O)NC8=O)cc7)CC6)CC5)c4)c3C[C@@H]1C2(F)F] |
| 208 | 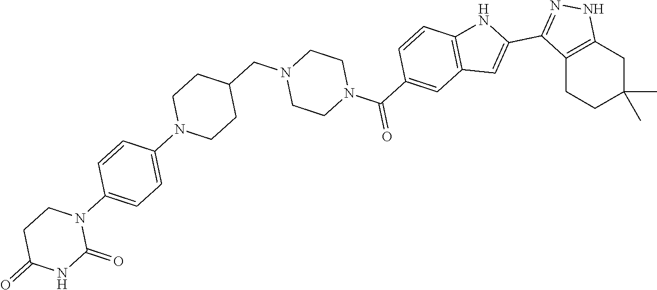 [SMILES: CC1(C)CCc2c(-c3cc4cc(C(=O)N5CCN(CC6CCN(c7ccc(N8CCC(=O)NC8=O)cc7)CC6)CC5)ccc4[nH]3)n[nH]c2C1] |
| 209 | 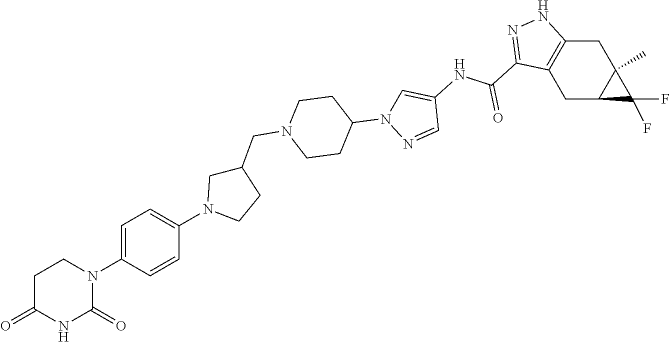 [SMILES: C[C@@]12Cc3[nH]nc(C(=O)Nc4cnn(C5CCN(CC6CCN(c7ccc(N8CCC(=O)NC8=O)cc7)C6)CC5)c4)c3C[C@@H]1C2(F)F] |
| 210 | 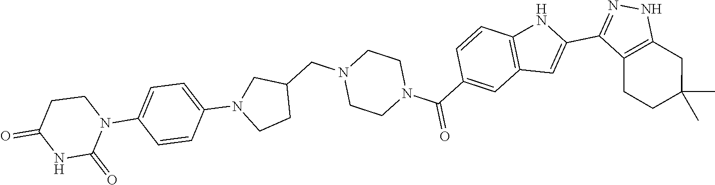 [SMILES: CC1(C)CCc2c(-c3cc4cc(C(=O)N5CCN(CC6CCN(c7ccc(N8CCC(=O)NC8=O)cc7)C6)CC5)ccc4[nH]3)n[nH]c2C1] |
| 211 | 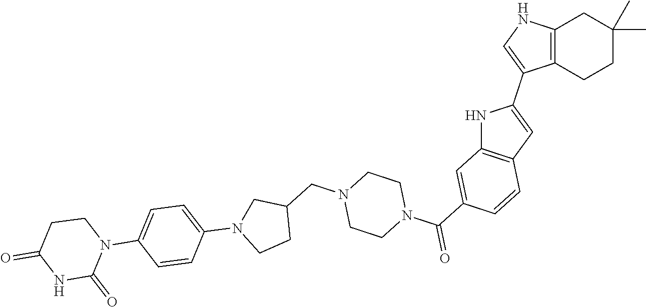 [SMILES: CC1(C)CCc2c(-c3cc4ccc(C(=O)N5CCN(CC6CCN(c7ccc(N8CCC(=O)NC8=O)cc7)C6)CC5)cc4[nH]3)c[nH]c2C1] |
| 212 | 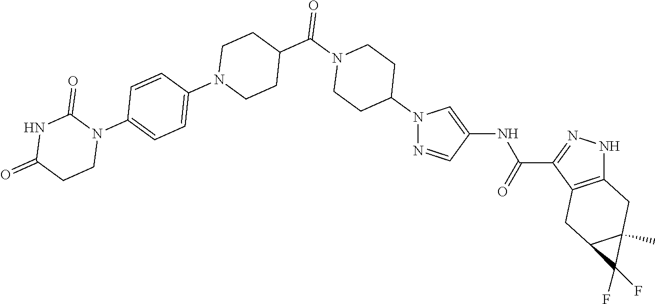 [SMILES: C[C@@]12Cc3[nH]nc(C(=O)Nc4cnn(C5CCN(C(=O)C6CCN(c7ccc(N8CCC(=O)NC8=O)cc7)CC6)CC5)c4)c3C[C@@H]1C2(F)F] |
| 213 | 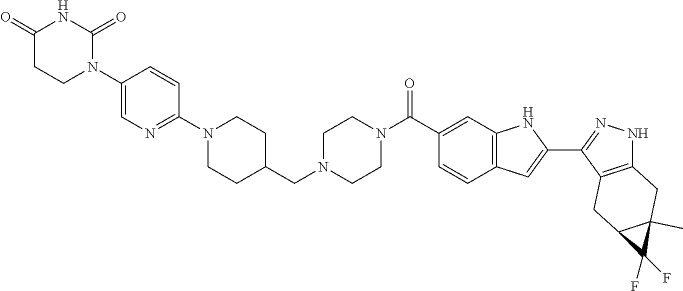 [SMILES: C[C@@]12Cc3[nH]nc(-c4cc5ccc(C(=O)N6CCN(CC7CCN(c8ccc(N9CCC(=O)NC9=O)cn8)CC7)CC6)cc5[nH]4)c3C[C@@H]1C2(F)F] |
| 214 | 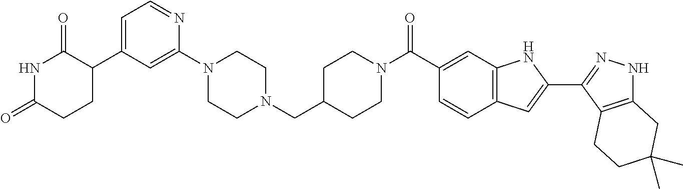 [SMILES: CC1(C)CCc2c(-c3cc4ccc(C(=O)N5CCC(CN6CCN(c7cc(C8CCC(=O)NC8=O)ccn7)CC6)CC5)cc4[nH]3)n[nH]c2C1] |
| 215 | 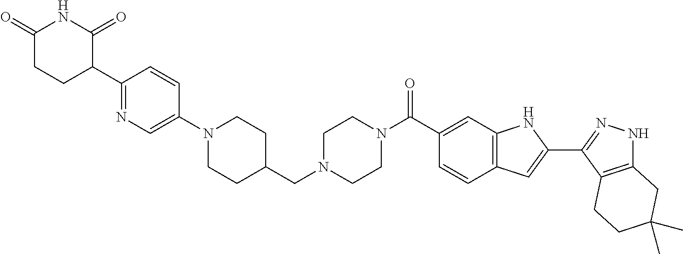 [SMILES: CC1(C)CCc2c(-c3cc4ccc(C(=O)N5CCN(CC6CCN(c7ccc(C8CCC(=O)NC8=O)nc7)CC6)CC5)cc4[nH]3)n[nH]c2C1] |
| 216 | 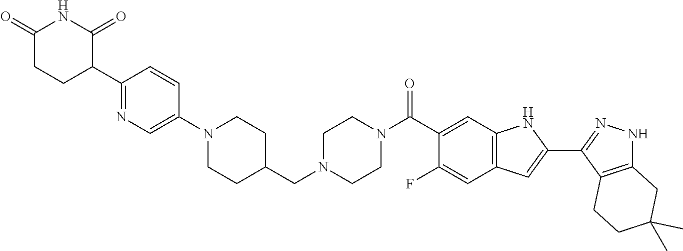 [SMILES: CC1(C)CCc2c(-c3cc4cc(F)c(C(=O)N5CCN(CC6CCN(c7ccc(C8CCC(=O)NC8=O)nc7)CC6)CC5)cc4[nH]3)n[nH]c2C1] |
| 217 | 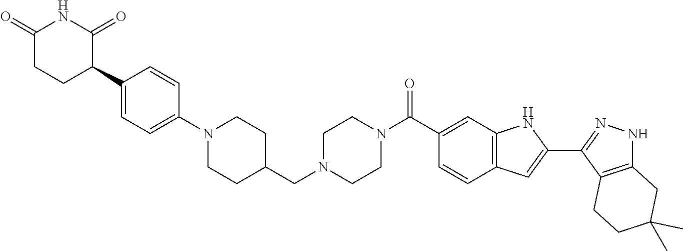 [SMILES: CC1(C)CCc2c(-c3cc4ccc(C(=O)N5CCN(CC6CCN(c7ccc([C@H]8CCC(=O)NC8=O)cc7)CC6)CC5)cc4[nH]3)n[nH]c2C1] |
| 218 | 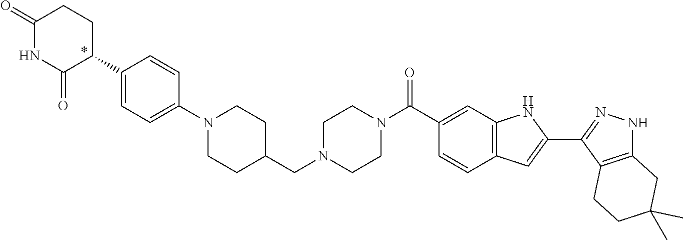 [SMILES: CC1(C)CCc2c(-c3cc4ccc(C(=O)N5CCN(CC6CCN(c7ccc([C@H]8CCC(=O)NC8=O)cc7)CC6)CC5)cc4[nH]3)n[nH]c2C1] |
| 219 | 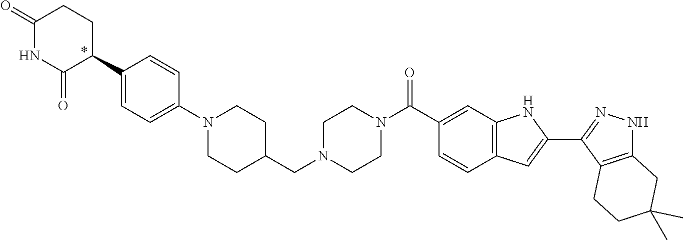 [SMILES: CC1(C)CCc2c(-c3cc4ccc(C(=O)N5CCN(CC6CCN(c7ccc([C@@H]8CCC(=O)NC8=O)cc7)CC6)CC5)cc4[nH]3)n[nH]c2C1] |
| 220 | 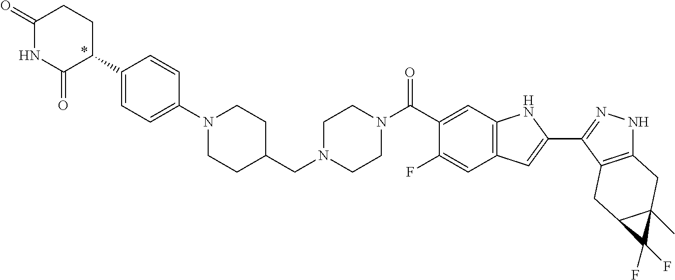 [SMILES: C[C@@]12Cc3[nH]nc(-c4cc5cc(F)c(C(=O)N6CCN(CC7CCN(c8ccc([C@H]9CCC(=O)NC9=O)cc8)CC7)CC6)cc5[nH]4)c3C[C@@H]1C2(F)F] |
| 221 | 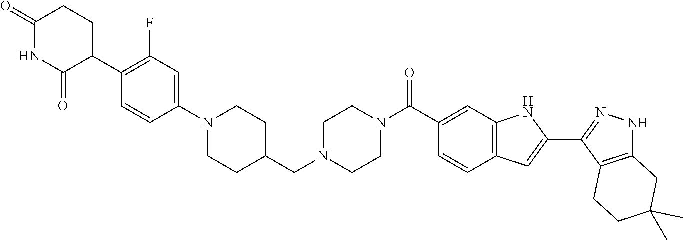 [SMILES: CC1(C)CCc2c(-c3cc4ccc(C(=O)N5CCN(CC6CCN(c7ccc(C8CCC(=O)NC8=O)c(F)c7)CC6)CC5)cc4[nH]3)n[nH]c2C1] |
| 222 | 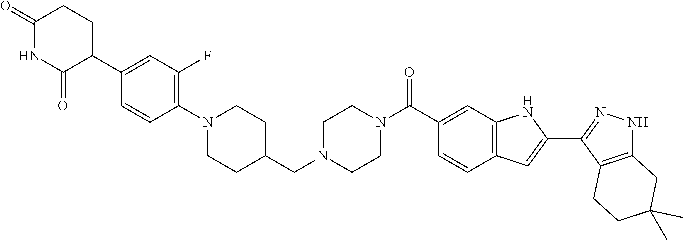 [SMILES: CC1(C)CCc2c(-c3cc4ccc(C(=O)N5CCN(CC6CCN(c7ccc(C8CCC(=O)NC8=O)cc7F)CC6)CC5)cc4[nH]3)n[nH]c2C1] |
| 223 | 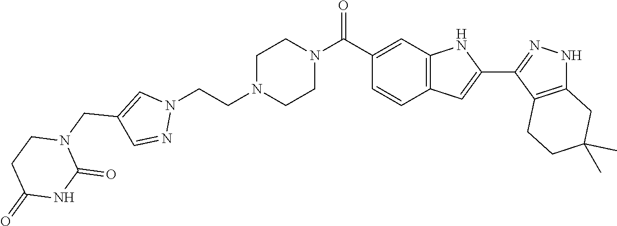 [SMILES: CC1(C)CCc2c(-c3cc4ccc(C(=O)N5CCN(CCn6cc(CN7CCC(=O)NC7=O)cn6)CC5)cc4[nH]3)n[nH]c2C1] |
| 224 | 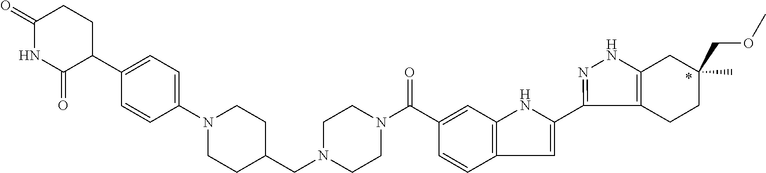 [SMILES: COC[C@@]1(C)CCc2c(-c3cc4ccc(C(=O)N5CCN(CC6CCN(c7ccc(C8CCC(=O)NC8=O)cc7)CC6)CC5)cc4[nH]3)n[nH]c2C1] |
| 225 | 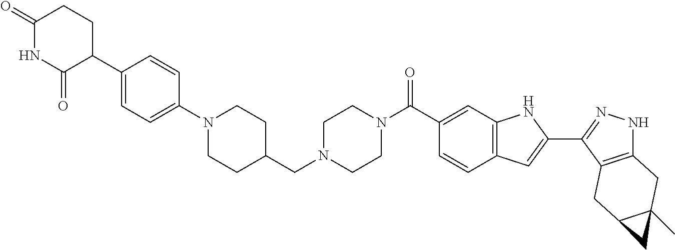 [SMILES: C[C@@]12Cc3[nH]nc(-c4cc5ccc(C(=O)N6CCN(CC7CCN(c8ccc(C9CCC(=O)NC9=O)cc8)CC7)CC6)cc5[nH]4)c3C[C@@H]1C2] |
| 226 | 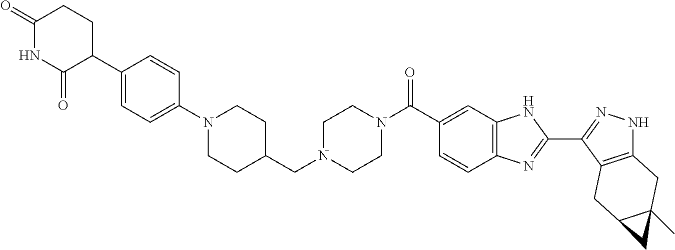 [SMILES: C[C@@]12Cc3[nH]nc(-c4nc5ccc(C(=O)N6CCN(CC7CCN(c8ccc(C9CCC(=O)NC9=O)cc8)CC7)CC6)cc5[nH]4)c3C[C@@H]1C2] |
| 227 | 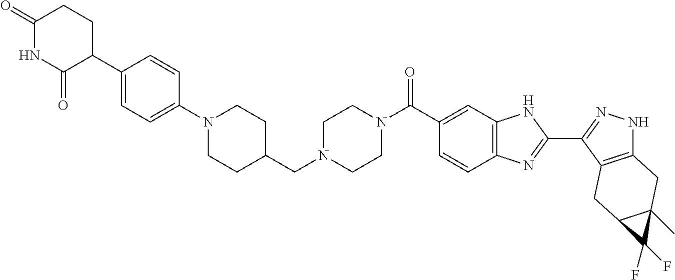 [SMILES: C[C@@]12Cc3[nH]nc(-c4nc5ccc(C(=O)N6CCN(CC7CCN(c8ccc(C9CCC(=O)NC9=O)cc8)CC7)CC6)cc5[nH]4)c3C[C@@H]1C2(F)F] |

In certain embodiments, the compound is selected from the compounds in Table 1.

### Pharmaceutical Compositions

The compounds described herein can be formulated into pharmaceutical compositions that further comprise a pharmaceutically acceptable carrier, diluent, adjuvant, or vehicle. In one embodiment, this disclosure provides a pharmaceutical composition comprising a compound described above, and a pharmaceutically acceptable carrier, diluent, adjuvant, or vehicle. In one embodiment, this disclosure is a pharmaceutical composition comprising an effective amount of a compound of this disclosure, or a pharmaceutically acceptable salt thereof and a pharmaceutically acceptable carrier, diluent, adjuvant, or vehicle. Pharmaceutically acceptable carriers include, for example, pharmaceutical diluents, excipients, or carriers suitably selected with respect to the intended form of administration, and consistent with conventional pharmaceutical practices.

According to another embodiment, the description provides a composition comprising a compound herein, or a pharmaceutically acceptable salt thereof, and a pharmaceutically acceptable carrier, adjuvant, or vehicle. Pharmaceutical compositions of this description comprise a therapeutically effective amount of a compound of Formula (I), (II), (III), (IIIa), (IV), (IVa), (V), (Va), (Vb), (VI), (VIa), and/or (VIb), wherein a “therapeutically effective amount” is an amount that is (a) effective to measurably degrade ITK (or reduce the amount of ITK) in a biological sample or in a patient; or (b) effective in treating and/or ameliorating a disease or disorder that is mediated by ITK.

The term “patient,” as used herein, means an animal, alternatively a mammal, and alternatively a human.

It also will be appreciated that certain compounds of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite, or residue thereof.

As used herein, the term “pharmaceutically acceptable salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic, and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion, or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions, and known techniques for the preparation thereof. Except insofar as any conventional carrier medium is incompatible with the compounds described herein, such as by producing any undesirable biological effect, or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic, or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade,” means a measurable reduction in (a) ITK activity, between a sample comprising a compound of this description and an ITK and an equivalent sample comprising an ITK in the absence of said compound; or (b) the concentration of the ITK in a sample over time.

### Administration

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate, and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a certain part of the intestinal tract, optionally, in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds, and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

Depending upon the particular condition, or disease, to be treated or prevented, additional therapeutic agents, which are normally administered to treat or prevent that condition, also may be present in the compositions of this disclosure. As used herein, additional therapeutic agents that are normally administered to treat or prevent a particular disease, or condition, are known as “appropriate for the disease, or condition, being treated.”

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically agent is a cancer agent (e.g., a biotherapeutic or chemo therapeutic cancer agent). In other embodiments, the additional therapeutically active agent is an anti-inflammatory agent.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### Methods of Use

The bifunctional compounds described herein are useful for degrading ITK in biological samples, or in patients via an ubiquitin proteolytic pathway. Thus, an embodiment of this disclosure provides a method of treating an ITK-mediated disease or disorder. As used herein, the term “ITK-mediated disease or disorder” means any disease, disorder, or other deleterious condition in which an ITK is known to play a role. In some instances, an ITK-mediated disease or disorder is a proliferative disorder, or an autoimmune disorder. Examples of proliferative disorders include cancer.

In one aspect, provided herein are methods of treating or preventing cancer in a subject in need thereof. In certain embodiments, the methods comprise the step of orally administering to the subject an amount of a bifunctional compound capable of inducing proteolytic degradation of ITK. In certain embodiments, the amount is effective to treat or prevent the cancer.

In certain embodiments, the cancer is any cancer described below. In particular embodiments, the cancer comprises a solid tumor. In certain embodiments, the cancer is a B cell malignancy. In certain embodiments, the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), and central nervous system (CNS) lymphoma. In certain embodiments, the cancer is chronic lymphocytic leukemia. In certain embodiments, the cancer is small cell lymphoma. In certain embodiments, the cancer is follicular lymphoma. In certain embodiments, the cancer is diffuse large B-cell lymphoma. In certain embodiments, the cancer is non-Hodgkin lymphoma. In certain embodiments, the cancer is mantle cell lymphoma. In certain embodiments, the cancer is marginal zone lymphoma. In certain embodiments, the cancer is Waldenstrom macroglobulinemia. In certain embodiments, the cancer is small lymphocytic lymphoma (SLL). In certain embodiments, the cancer is CNS lymphoma. In certain embodiments, the cancer is transformed CLL or Richter's transformation.

In another aspect, provided herein are methods of degrading ITK in a subject in need thereof. The methods comprise the step of orally administering to the subject an amount of a bifunctional compound capable of inducing proteolytic degradation of ITK. In certain embodiments, the amount is effective to degrade ITK in the subject. The ITK can be expressed in any cells or tissues of the subject. In certain embodiments, the ITK is expressed in splenocytes. In certain embodiments, the ITK is expressed in peripheral blood mononuclear cells.

In another aspect, provided herein are methods of preventing B cell activation in a subject in need thereof. The methods comprise the step of orally administering to the subject an amount of a bifunctional compound capable of inducing proteolytic degradation of ITK. In certain embodiments, the amount is effective to prevent B cell activation. In certain embodiments, the B cell expresses CD69. In certain embodiments, the B cell expresses CD86. In certain embodiments, the B cell expresses CD69 and CD86.

In the methods, the bifunctional compounds comprise a moiety capable of specifically binding ITK and further comprise a moiety capable of recruiting an ubiquitin ligase to degrade the ITK. Particular compounds are described herein. The compounds can be administered in any form, including pharmaceutically acceptable salt and pharmaceutical compositions.

The bifunctional compound can be administered in any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg. In certain embodiments, the dose is selected from the group consisting of 100 mg/kg, 200 mg/kg, 300 mg/kg, 450 mg/kg, 600 mg/kg, 800 mg/kg, and 1000 mg/kg. In certain embodiments, the dose is about 25 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The dose can be administered on a schedule deemed suitable by the person of skill in the art. In certain embodiments, the dose is administered once per day. In certain embodiments, the dose is administered twice per day. In certain embodiments, the dose is administered three times per day. In certain embodiments, the dose is administered four times per day. In certain embodiments, the dose is administered in divided doses. In certain embodiments, the dose is administered in two divided doses per day. In certain embodiments, the dose is administered in three divided doses per day. In certain embodiments, the dose is administered in four divided doses per day.

Dosing can continue for any length of time deemed suitable by the person of skill in the art. In certain embodiments, the dose is administered daily for fourteen days. In certain embodiments, the dose is administered daily for thirteen days. In certain embodiments, the dose is administered daily for twelve days. In certain embodiments, the dose is administered daily for eleven days. In certain embodiments, the dose is administered daily for ten days. In certain embodiments, the dose is administered daily for nine days. In certain embodiments, the dose is administered daily for eight days. In certain embodiments, the dose is administered daily for seven days. In certain embodiments, the dose is administered daily for six days. In certain embodiments, the dose is administered daily for five days. In certain embodiments, the dose is administered daily for four days. In certain embodiments, the dose is administered daily for three days. In certain embodiments, the dose is administered daily for two days. In certain embodiments, the dose is administered for one day.

In the dosing schedule, the doses can be administered on consecutive days or cyclicly, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the dose is administered weekly. In certain embodiments, the dose is administered twice per week. In certain embodiments, the dose is administered three times per week.

In certain embodiments, the dose(s) are administered for a period of time with a first interval between dose(s), and then the dose(s) are re-administered for a period of time following the first interval between dose(s), wherein this dosing regimen can be repeated (i.e., cyclicly or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered by any route of administration deemed suitable by the practioner of skill. In certain embodiments, the dose is administered orally. Formulations and techniques for administration are described in detail below.

In certain embodiments, term “cancer” includes, but is not limited to, the following cancers: epidermoid Oral: buccal cavity, lip, tongue, mouth, pharynx, squamous cell carcinoma of the head and neck (HNSCC); Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); Gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma (MM), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenstrom's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis; Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metatstaic melanoma.

In certain embodiments, term “autoimmune disease” includes, but is not limited to, the following autoimmune diseases: uticaria, graft-versus-host disease (GVHD), acute graft-versus-host disease, pemphigus vulgaris, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, axonal and neuronal neuropathy (AMAN), Balö disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive Arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopenic purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)).

In certain embodiments, term “inflammatory disease” includes, but is not limited to, the following inflammatory diseases: encephalitis, myelitis, meningitis, arachnoiditis, neuritis, dacryoadenitis, scleritis, episcleritis, keratitis, retinitis, chorioretinitis, blepharitis, conjunctivitis, uveitis, otitisexterna, otitismedia, labyrinthitis, mastoiditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascendingcholangitis, cholecystitis, pancreatitis, peritonitis, dermatitis, folliculitis, cellulitis, hidradenitis, arthritis, dermatomyositis, softtissue, myositis, synovitis/tenosynovitis, bursitis, enthesitis, fasciitis, capsulitis, epicondylitis, tendinitis, panniculitis, osteochondritis:osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminalvesiculitis, balanitis, posthitis, balanoposthitis, chorioamnionitis, funisitis, omphalitis, insulitis, hypophysitis, thyroiditis, parathyroiditis, adrenalitis, lymphangitis, and lymphadenitis.

### Articles of Manufacture and Kits.

Also provided are articles of manufacture comprising any of the compounds or pharmaceutical compositions described herein. The articles of manufacture include suitable containers or packaging materials for the compounds or pharmaceutical compositions. Examples of a suitable container include, but are not limited to, a bottle, a vial, a syringe, an intravenous bag, or a tube.

Also provided are kits comprising any of the compounds or pharmaceutical compositions described herein. The kits can contain the compounds or pharmaceutical compositions in suitable containers or packaging materials, including, but not limited to, a bottle, a vial, a syringe, an intravenous bag, or a tube. The kits can comprise the compounds or pharmaceutical compositions for administration to an individual in single-dose form or in multiple-dose form. The kits can further comprise instructions or a label for administering the compounds or pharmaceutical compositions to an individual according to any of the methods disclosed herein. The kits can further comprise equipment for administering the compounds or pharmaceutical compositions to an individual, including, but not limited to, needles, syringes, tubing, or intravenous bags. The kits can further comprise instructions for producing any of the compounds or pharmaceutical compositions disclosed herein.

Also provided are articles of manufacture comprising any of the compounds, vaccines, or pharmaceutical compositions described herein. The articles of manufacture include suitable containers or packaging materials for the compounds or pharmaceutical compositions. The articles of manufacture include suitable containers or packaging materials for the compounds, oncolytic viruses, or pharmaceutical compositions. Examples of a suitable container include, but are not limited to, a bottle, a vial, a syringe, an intravenous bag, or a tube.

The disclosure will be more fully understood by reference to the following examples. They should not, however, be construed as limiting the scope of this disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional embodiments are disclosed in further detail in the following examples, which are not in any way intended to limit the scope of the claims.

### Analytical Methods and Instrumentation

Proton nuclear magnetic resonance (NMR) spectra were obtained on Bruker Ascend™ 500 MHz spectrometer. NMR spectra are reported as follows: chemical shift δ (ppm), multiplicity, coupling constant J(Hz), and integration. The abbreviations s=singlet, d=doublet, t=triplet, q=quartet, m=multiplet and br=broad are used throughout. Mass spectral data were measured using the following systems: Waters Acquity i-class ultra-performance liquid chromatography (UPLC) system with Acquity Photo Diode Array Detector, Acquity Evaporative Light Scattering Detector (ELSD) and Waters ZQ Mass Spectrometer. Data was acquired using Waters MassLynx 4.1 software and purity characterized by UV wavelength 220 nm, evaporative light scattering detection (ELSD) and electrospray positive ion (ESI) (column: Acquity UPLC BEH C18 1.7μ τ 2.1×50 mm). Solvents used: acetonitrile/water, containing 0.1% formic acid; flow rate 0.7 mL/min. Preparatory HPLC purifications were conducted with a flow rate of 15 mL/min and detection by UV wavelength 214 nm and 254 nm (Column: Jupiter© 10 μM Proteo 90 Å, 250×21.2 mm A, solvent: acetonitrile/water, containing modifier such as 0.1% trifluoroacetic acid).”

Abbreviations used in the examples include:

| Abbreviation | Name |
|---|---|
| CH₃CN | acetonitrile |
| aq. | aqueous |
| atm | atmospheres |
| BINAP | (1,1′-binaphthalene-2,2′-diyl)bis(di- |
| | phenylphosphine) |
| Boc | t-butoxycarbonyl |
| CCl₄ | carbon tetrachloride |
| CDCl₃ | deuterated chloroform |
| CO | carbon monoxide gas |
| CO₂ | carbon dioxide |
| Cs₂CO₃ | cesium carbonate |
| CuBr | copper(I) bromide |
| Cu(OAc)₂ | copper(II) acetate |
| DCM | dichloromethane |
| DEAD | diethyl azodicarboxylate |
| DIPEA | diisopropylethylamine |
| DMF | N,N-dimethylformamide |
| DMSO | dimethylsulfoxide |
| ESI | electrospray ionization |
| Et₃N | triethylamine |
| EtOAc | ethyl acetate |
| EtOH | ethanol |
| h | hours |
| H₂O | water |
| HATU | 1-[bis(dimethylamino)methylene]-1H- |
| | 1,2,3-triazolo[4,5-b]pyridinium 3- |
| | oxide hexafluorophosphate |
| HCl | hydrogen chloride |
| HOAc | acetic acid |
| K₂CO₃ | potassium carbonate |
| KI | potassium iodide |
| KOH | potassium hydroxide |
| KOtBu | potassium tert-butoxide |
| LiAlH₄ | lithium aluminum hydride |
| LiOH | lithium hydroxide |
| MeOH | methanol |
| min | minutes |
| N₂ | nitrogen |
| Na₂CO₃ | sodium carbonate |
| Na₂SO₄ | sodium sulfate |
| NaH | sodium hydride |
| NaHCO₃ | sodium bicarbonate |
| NaOH | sodium hydroxide |
| NBS | N-bromosuccinimide |
| NH₄Cl | ammonium chloride |
| NMR | nuclear magnetic resonance |
| Pd₂(dba)₃ | tris(dibenzylideneacetone)dipalladium(0) |
| Pd(dppf)Cl₂ | [1,1′-bis(diphenylphosphino)ferrocene]di- |
| | chloropalladium(II) |
| Pd/C | palladium on carbon |
| Pd(OAc)₂ | palladium(II) acetate |
| Prep-TLC | preparatory thin layer chromatography |
| RuPhos-Pd- | chloro(2-dicyclohexylphosphino-2′,6′- |
| G2 | diisopropoxy-1,1′-biphenyl)[2-(2′- |
| | amino-1,1′-biphenyl)]palladium(II) |
| sat. | saturated |
| t-BuOH | tert-butanol |
| THF | Tetrahydrofuran |
| TLC | Thin layer chromatography |
| Pre-TLC | Pre-Thin layer chromatography |

### General Schemes For Preparing LHP Building Blocks

CRBN-targeting LHM can be generally prepared according to Scheme B1

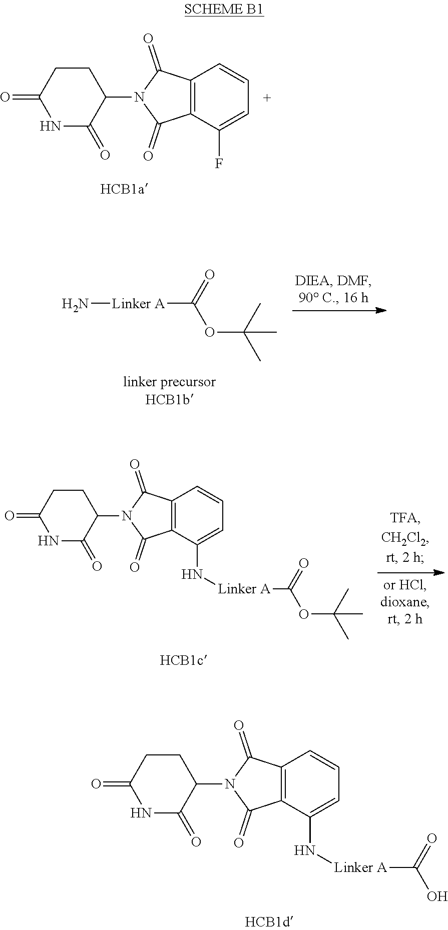

In Scheme B1, a functionalized thalidomide was first coupled to a linker precursor. The linker precursor (an amino tert-butyl ester) comprises “linker A” (i.e., representing one or more linker segments, including L₅) and two terminal reactive groups, the amine and the protected carboxylic acid in ester form. Step 1 below describes in more detail the initial coupling step using the exemplary aminoester linker precursor.

Step 1: A mixture of 2-(2,6-dioxopiperidin-3-yl)-4-fluoro-2,3-dihydro-1H-isoindole-1,3-dione (0.26 mmol), aminoester (0.26 mmol), ethylbis(propan-2-yl)amine (0.52 mmol) and DMF (1 mL) was allowed to stir at 90° C. overnight. The mixture was cooled and purified by HPLC (5-95% MeCN in H₂O with 0.1% TFA) to afford the tert-butyl ester intermediate.

The tert-butyl ester intermediate was then hydrolyzed (see Step 2) to provide a CRBN-targeting LHP building block having “linker A” terminating in a carboxylic acid, which may be further coupled to another moiety.

Step 2: A mixture of tert-butyl 4-{[2-(2,6-dioxopiperidin-3-yl)-1,3-dioxo-2,3-dihydro-1H-isoindol-4-yl]amino}butanoate (0.10 mmol), CH₂Cl₂ (1 mL), and TFA (1 mL) was allowed to stir at room temperature for 2 h. The mixture was concentrated to afford the carboxylic acid product.

Described below are additional examples of CRBN-targeting LHM building blocks that may be prepared according to Scheme B1.

### Example 1. 3-(2-((2-(2,6-dioxopiperidin-3-yl)-1,3-dioxoisoindolin-4-yl)amino)ethoxy)propanoic acid (HCB1)

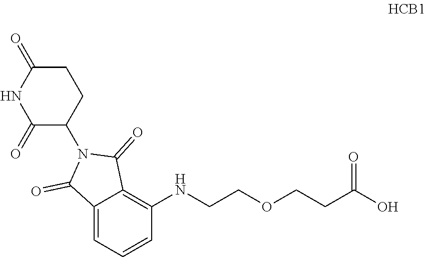 [SMILES: O=C(O)CCOCCNc1cccc2c1C(=O)N(C1CCC(=O)NC1=O)C2=O]

Step 1 product: tert-butyl 3-[2-[[2-(2,6-dioxo-3-piperidyl)-1,3-dioxo-isoindolin-4-yl]amino]ethoxy]propanoate (1.8 g, 51.9%). LCMS: C₂₂H₂₇N₃O₇ requires: 445, found: m/z=468 [M+Na]⁺.

Step 2 product: 3-[2-[[2-(2,6-dioxo-3-piperidyl)-1,3-dioxo-isoindolin-4-yl]amino]ethoxy]propanoic acid (HCB1) (526 mg, 32%). LCMS: C₁₈H₁₉N₃O₇ requires: 389, found: m/z=390 [M+H]⁺.

### Example 2. 3-(2-(2-(2-((2-(2,6-dioxopiperidin-3-yl)-1,3-dioxoisoindolin-4-yl)amino)ethoxy)ethoxy)ethoxy)propanoic acid (HCB2)

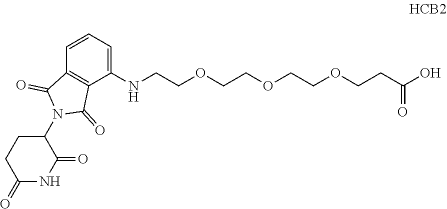 [SMILES: O=C(O)CCOCCOCCOCCNc1cccc2c1C(=O)N(C1CCC(=O)NC1=O)C2=O]

Step 1 product: tert-butyl 3-[2-[2-[2-[[2-(2,6-dioxo-3-piperidyl)-1,3-dioxoisoindolin-4-yl]amino]ethoxy]ethoxy]ethoxy]propanoate (1.6 g, 41%). LCMS: C₂₆H₃₅N₃O₉ requires: 533, found: m/z=534 [M+H]⁺.

Step 2 product: 3-[2-[2-[2-[[2-(2,6-dioxo-3-piperidyl)-1,3-dioxo-isoindolin-4-yl]amino]ethoxy]ethoxy]ethoxy]propanoic acid (HCB2) (1.2 g, 73.62%). LCMS: C₂₂H₂₇N₃O₉ requires: 477, found: m/z=478 [M+H]⁺.

### Example 3. 3-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]propanic acid (HCB3)

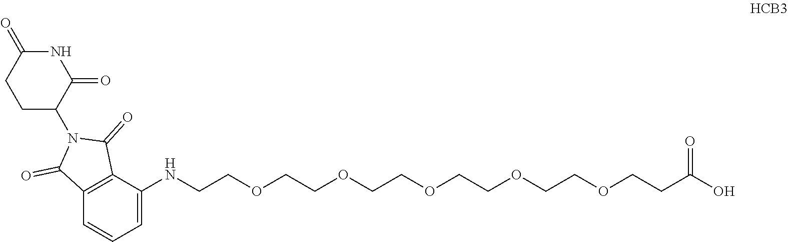 [SMILES: O=C(O)CCOCCOCCOCCOCCOCCNc1cccc2c1C(=O)N(C1CCC(=O)NC1=O)C2=O]

Step 1 product: 2-(2,6-dioxo-3-piperidyl)-4-fluoro-isoindoline-1,3-dione (1.75 g, 6.32 mmol) treated with tert-butyl 3-[2-[2-[2-[2-(2-aminoethoxy)ethoxy]ethoxy]ethoxy]ethoxy]propanoate (2.1 g, 5.75 mmol) provided tert-butyl 3-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]propanoate (1.8 g, 2.9 mmol, 50.39%) as a yellow oil. LCMS: C₃₀H₄₃N₃O₁₁ requires: 621, found: m/z=622 [M+H]⁺.

Step 2 product: Starting from tert-butyl 3-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]propanoate (1.8 g, 2.9 mmol) provided the desired product (HCB3) (1.2 g, 2.07 mmol, 71.51%) as a yellow oil. LCMS: C₂₆H₃₅N₃O₁₁ requires: 565, found: m/z=566 [M+H]⁺.

### Example 4. 3-[2-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]ethoxy]ethoxy]propanic acid (HCB4)

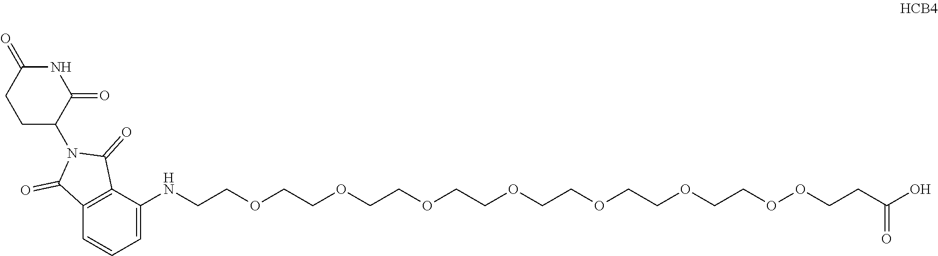 [SMILES: O=C(O)CCOOCCOCCOCCOCCOCCOCCOCCNc1cccc2c1C(=O)N(C1CCC(=O)NC1=O)C2=O]

Step 1 product: 2-(2,6-dioxo-3-piperidyl)-4-fluoro-isoindoline-1,3-dione (2 g, 7.24 mmol) treated with tert-butyl 3-[2-[2-[2-[2-[2-[2-[2-(2-aminoethoxy)ethoxy]ethoxy]ethoxy]ethoxy]ethoxy]ethoxy]ethoxy]propanoate (3.6 g, 7.24 mmol) provided tert-butyl 3-[2-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]ethoxy]ethoxy]propanoate (1.5 g, 1.93 mmol, 26.66%) as a yellow oil. LCMS: C₃₆H₅₅N₃O₁₄ requires: 753, found: m/z=754 [M+H]⁺.

Step 2 product: Starting from tert-butyl 3-[2-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]ethoxy]ethoxy]propanoate (HCB4) (1.5 g, 1.93 mmol) provided the desired product (1.21 g, 1.53 mmol, 80%) as a yellow gum. LCMS: C₃₂H₄₇N₃O₁₄ requires: 697, found: m/z=698 [M+H]⁺.

### Example 5. 6-((2-(2,6-dioxopiperidin-3-yl)-1,3-dioxoisoindolin-5-yl)amino)hexanoic acid (HCB5)

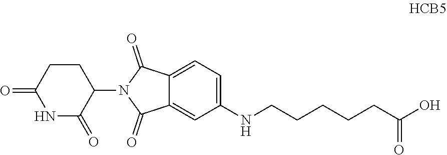 [SMILES: O=C(O)CCCCCNc1ccc2c(c1)C(=O)N(C1CCC(=O)NC1=O)C2=O]

Step 1: tert-butyl 6-{[2-(2,6-dioxopiperidin-3-yl)-1,3-dioxoisoindol-5-yl]amino}hexanoate

To a mixture of 2-(2,6-dioxopiperidin-3-yl)-5-fluoroisoindole-1,3-dione (250 mg, 0.91 mmol), tert-butyl 6-aminohexanoate hydrochloride (203 mg, 0.91 mmol) in NMP (3 mL), was added N,N-diisopropylethylamine (0.6 mL) with heating to 85° C. overnight. The crude reaction mixture was purified by silica gel chromatography using EtOAc:Hexane (0-100%), to give tert-butyl 6-{[2-(2,6-dioxopiperidin-3-yl)-1,3-dioxoisoindol-5-yl]amino}hexanoate (111 mg, 28%). LCMS: C₂₃H₂₉N₃O₆, requires: 443.5, found: m/z=444.4 [M⁺H]⁺.

Step 2: 6-((2-(2,6-dioxopiperidin-3-yl)-1,3-dioxoisoindolin-5-yl)amino)hexanoic acid To a solution of tert-butyl 6-{[2-(2,6-dioxopiperidin-3-yl)-1,3-dioxoisoindol-5-yl]amino}hexanoate (111 mg, 0.25 mmol) in DCM (2 mL) was added TFA (0.5 mL). The reaction mixture was stirred at room temperature for 30 min, and then the reaction mixture was concentrated to give 6-((2-(2,6-dioxopiperidin-3-yl)-1,3-dioxoisoindolin-5-yl)amino)hexanoic acid (HCB5) (78 mg, 78%). ¹H NMR (500 MHz, DMSO-d₆) δ 12.00 (s, 1H), 11.06 (s, 1H), 7.57 (d, J=8.3 Hz, 1H), 7.11 (s, 1H), 6.95 (d, J=2.1 Hz, 1H), 6.85 (dd, J=8.4, 2.1 Hz, 1H), 5.04 (dd, J=12.7, 5.4 Hz, 1H), 3.16 (q, J=6.4 Hz, 2H), 2.23 (t, J=7.4 Hz, 2H), 2.03-1.97 (m, 1H), 1.56 (dq, J=14.8, 7.2 Hz, 4H), 1.39 (q, J=7.9 Hz, 2H). LCMS: C₁₉H₂₁N₃O₆ requires: 387.4, found: m/z=388.4 [M+H]⁺.

### Example 6. 3-{4-[2-(2,6-dioxopiperidin-3-yl)-1,3-dioxoisoindol-5-yl]piperazin-1-yl}propanoic acid (HCB6)

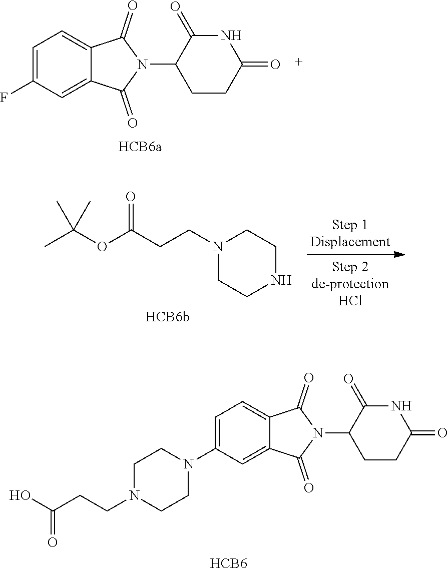

### Step 1: tert-butyl 3-(4-(2-(2,6-dioxopiperidin-3-yl)-1,3-dioxoisoindolin-5-yl)piperazin-1-yl)propanoate

To a solution of tert-butyl 3-(piperazin-1-yl)propanoate (HCB6b) (400.00 mg, 1.87 mmol) and 2-(2,6-dioxopiperidin-3-yl)-5-fluoroisoindole-1,3-dione (HCB6a) (515.56 mg, 1.87 mmol) in NMP (10 mL) was added N,N-diisopropylethylamine (0.65 mL, 0.48 g, 3.73 mmol) with heating at 85-90° C. for 16 hr. The reaction was partitioned between EtOAC:water (2×). The organic layer was then washed with brine, dried over sodium sulfate, and concentrated. Silica gel column purification using 10-100% EtOAc:Hexanes, provided the desired product (823 mg). LCMS: C₂₄H₃₀N₄O₆ requires: 470.5, found: m/z=471.8 [M+H]⁺.

### Step 2: 3-{4-[2-(2,6-dioxopiperidin-3-yl)-1,3-dioxoisoindol-5-yl]piperazin-1-yl}propanoic acid (HCB6)

Tert-butyl 3-{4-[2-(2,6-dioxopiperidin-3-yl)-1,3-dioxoisoindol-5-yl]piperazin-1-yl}propanoate (820.00 mg, 1.74 mmol) was dissolved in trifluoroacetic acid (9.94 g, 87.14 mmol), and after one hour the TFA was removed. Lyophilization provided 3-{4-[2-(2,6-dioxopiperidin-3-yl)-1,3-dioxoisoindol-5-yl]piperazin-1-yl}propanoic acid (HCB6) (722 mg, 100%). LCMS: C₂₀H₂₂N₄O₆ requires: 414.4, found: m/z=415.4 [M+H]⁺.

### Example 7. 2-(2-(2-(2,6-dioxopiperidin-3-yl)-1,3-dioxoisoindolin-5-yl)-2,7-diazaspiro[3.5]nonan-7-yl)acetic acid (HCB7)

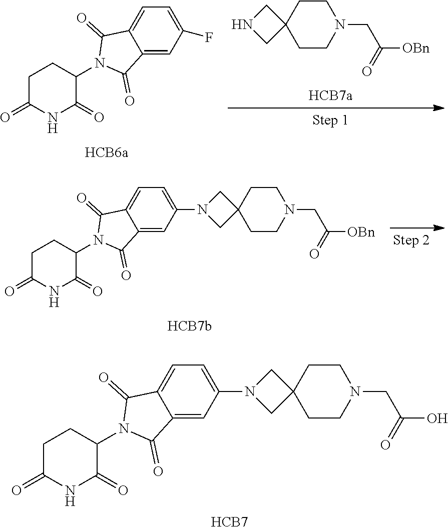

### Step 1: benzyl 2-{2-[2-(2,6-dioxopiperidin-3-yl)-1,3-dioxoisoindol-5-yl]-2,7-diazaspiro[3.5]nonan-7-yl}acetate (HCB7b)

To a mixture of 2-(2,6-dioxopiperidin-3-yl)-5-fluoroisoindole-1,3-dione (HCB6a) (70.00 mg, 0.25 mmol) and benzyl 2-{2,7-diazaspiro[3.5]nonan-7-yl}acetate (HCB7a) (69.53 mg, 0.25 mmol) in NMP (2 mL) was added N,N-diisopropylethylamine (0.13 mL) and the reaction was heated to 85° C. overnight. The crude mixture was purified by column chromatography eluting with EtOAc:Hexane (10-100%) to give benzyl 2-{2-[2-(2,6-dioxopiperidin-3-yl)-1,3-dioxoisoindol-5-yl]-2,7-diazaspiro[3.5]nonan-7-yl}acetate (HCB7b) (68 mg, 51%). LCMS: C₂₉H₃₀N₄O₆ requires: 530, found: m/z=531 [M+H]⁺.

### Step 2: 2-(2-(2-(2,6-dioxopiperidin-3-yl)-1,3-dioxoisoindolin-5-yl)-2,7-diazaspiro[3.5]nonan-7-yl)acetic acid (HCB7)

To a solution of benzyl 2-{2-[2-(2,6-dioxopiperidin-3-yl)-1,3-dioxoisoindol-5-yl]-2,7-diazaspiro[3.5]nonan-7-yl}acetate (HCB7b) (68.00 mg, 0.13 mmol) in EtOH (5 mL) and DCM (2 mL) was added Palladium on carbon (6 mg, 0.06 mmol). The reaction mixture was sparged with hydrogen and kept under one atmosphere of hydrogen using a balloon. The reaction was stirred at room temperature for 48 h. The reaction mixture was then filtered through a pad of Celite and concentrated to give benzyl 2-{2-[2-(2,6-dioxopiperidin-3-yl)-1,3-dioxoisoindol-5-yl]-2,7-diazaspiro[3.5]nonan-7-yl}acetate (HCB7b) (56 mg, 99%). LCMS: C₂₂H₂₄N₄O₆ requires: 440, found: m/z=441 [M+H]⁺.

### Example 8. 3-(2-((2-(2,6-dioxopiperidin-3-yl)-1,3-dioxoisoindolin-5-yl)amino)ethoxy)propanoic acid (HCB8)

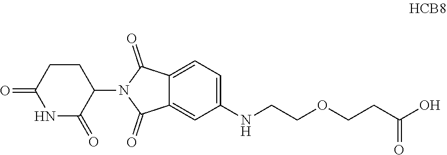 [SMILES: O=C(O)CCOCCNc1ccc2c(c1)C(=O)N(C1CCC(=O)NC1=O)C2=O]

HCB8 was prepared in a similar manner to HCB1 by substituting 2-(2,6-dioxopiperidin-3-yl)-5-fluoroisoindoline-1,3-dione for 2-(2,6-dioxopiperidin-3-yl)-4-fluoroisoindoline-1,3-dione to give the title compound. LCMS: C₁₈H₁₉N₃O₇ requires: 389.1, found: m/z=387.8 [M−H]⁻.

### Example 9. 1-(1-(2-(2,6-dioxopiperidin-3-yl)-1,3-dioxoisoindolin-5-yl)piperidin-4-yl)azetidine-3-carboxylic acid (HCB9)

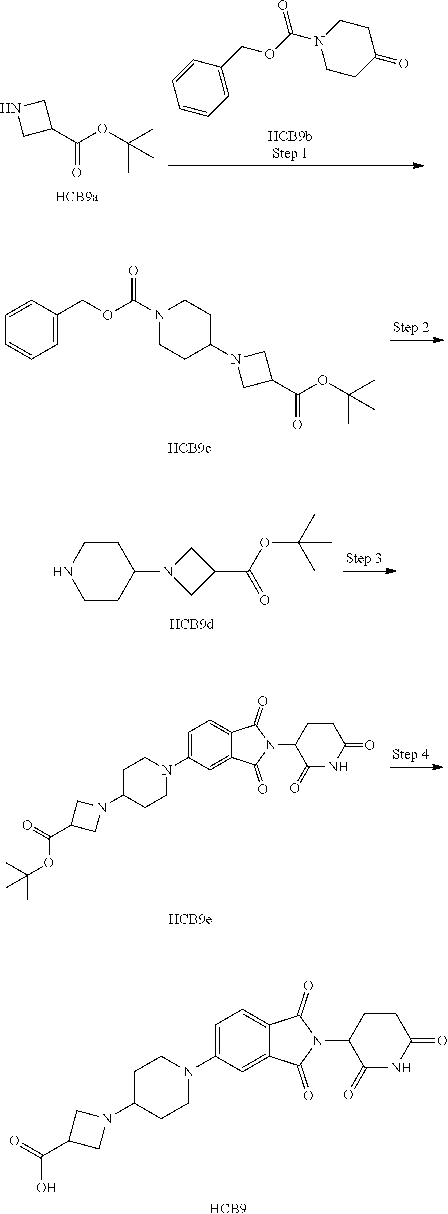

### Step 1: benzyl 4-{3-[(tert-butoxy)carbonyl]azetidin-1-yl}piperidine-1-carboxylate (HCB9c)

To a solution of tert-butyl azetidine-3-carboxylate (4.5 g, 28.62 mmol, 1.0 equiv) (HCB9a) and 1-(benzyloxycarbonyl)-4-piperidinone (HCB9b) (7.35 g, 31.49 mmol, 1.10 equiv) in DCE (136 mL, 0.2 M) was added acetic acid (2.46 mL, 42.94 mmol, 1.5 equiv) and the reaction was stirred at room temperature for one hour. Then NaBH(OAc)₃ (9.71 g, 45.8 mmol, 1.6 equiv) was added and the reaction was stirred at room temperature overnight. The reaction mixture was quenched with aqueous NaHCO₃, extracted with DCM (3×), washed with brine, dried over Na₂SO₄ and concentrated to dryness. The colorless oil was purified by flash column chromatography eluted with DCM:MeOH (0-10%) to give the desired product as a white solid (9.39 g, 88% yield). ESI [M+H]⁺=375.6

### Step 2: tert-butyl 1-(piperidin-4-yl)azetidine-3-carboxylate (HCB9d)

A solution of benzyl 4-{3-[(tert-butoxy)carbonyl]azetidin-1-yl}piperidine-1-carboxylate (HCB9c) (9.39 g, 25.07 mmol, 1.0 equiv) in MeOH (250 mL, 0.1 M) was degassed and back-filled with argon three times. Then Pd(OH)₂ (0.7 g, 5.0 mmol, 0.2 equiv) was added and the mixture was again degassed and back-field with argon three times. Then the reaction was degassed and charged with H₂ via a balloon and stirred at room temperature overnight. The reaction was monitored by UPLC which confirmed Cbz cleavage. The reaction mixture was then filtered through a celite pad and the filtrate was concentrated to afford the desired product (5.81 g, 96%).

### Step 3: tert-butyl 1-{1-[2-(2,6-dioxopiperidin-3-yl)-1,3-dioxo-2,3-dihydro-1H-isoindol-5-yl]piperidin-4-yl}azetidine-3-carboxylate (HCB9e)

To a solution of 2-(2,6-dioxopiperidin-3-yl)-5-fluoro-2,3-dihydro-1H-isoindole-1,3-dione (HCB6a) (6.05 g, 21.9 mmol, 1.0 equiv) in DMSO (43.8 mL, 0.5 M) was added tert-butyl 1-(piperidin-4-yl)azetidine-3-carboxylate (HCB9d) (5.79 g, 24.09 mmol, 1.1 equiv) and DIPEA (7.63 mL, 43.8 mmol, 2.0 equiv). The reaction mixture was then moved to pre-heated bath at 90° C., and stirred overnight under an argon atmosphere. The reaction was monitored by UPLC and showed the formation of the desired product. The reaction mixture was quenched with water, extracted with DCM (3×), and the organic phase was washed with ice-cold water. The crude material was purified by flash chromatography eluted with DCM:Acetone (0-10%) to provide the product (HCB9e) as a yellow solid (6.95 g, 64%). ESI [M+H]⁺=497.4.

### Step 4: 1-{1-[2-(2,6-dioxopiperidin-3-yl)-1,3-dioxo-2,3-dihydro-1H-isoindol-5-yl]piperidin-4-yl}azetidine-3-carboxylic acid hydrochloride (HCB9.HCl)

To a solution of tert-butyl 1-{1-[2-(2,6-dioxopiperidin-3-yl)-1,3-dioxo-2,3-dihydro-1H-isoindol-5-yl]piperidin-4-yl}azetidine-3-carboxylate (HCB9e) (4.95 g, 9.97 mmol, 1.0 equiv) in anhydrous DCM (100 mL, 0.1 M) was added 2 M HCl in Et₂O (50 mL, 99.69 mmol, 10.0 equiv). The reaction mixture was then stirred at room temperature for 2 h. The reaction was monitored by UPLC and showed that starting material remained. Another portion of HCl in Et₂O (50 mL, 99.69 mmol, 10.0 equiv) was added and the reaction mixture stirred for another 3 h. UPLC showed 10% of the starting material remained. The precipitate was filtered and the crude material was dissolved in DCM followed by the addition of 2 M HCl in Et₂O (50 mL, 99.69 mmol, 10.0 equiv). The reaction was sonicated for 45 min. The precipitated solids were filtered, washed with Et₂O, and dried under vacuum to provide the desired product as an HCl salt (4.83 g, quant). ¹H NMR (300 MHz, D₂O) δ 7.68 (d, J=8.5 Hz, 1H), 7.37 (s, 1H), 7.22 (dd, J=8.6, 2.3 Hz, 1H), 5.14 (dd, J=12.8, 5.6 Hz, 1H), 4.49-4.28 (m, 4H), 4.08 (d, J=13.6 Hz, 2H), 3.80 (t, J=9.0 Hz, 1H), 3.69-3.56 (m, 1H), 3.03 (t, J=12.8 Hz, 2H), 2.92-2.73 (m, 2H), 2.61 (qd, J=12.8, 5.6 Hz, 1H), 2.24-2.09 (m, 2H), 1.59-1.42 (m, 2H). LCMS: (254 nm) Rt=2.83 min, 94.5%, ESI [M+H]⁺=441.07.

### Example 10. 2-(4-(2-(2,6-dioxopiperidin-3-yl)-1,3-dioxoisoindolin-5-yl)piperazin-1-yl)acetic acid (HCB10)

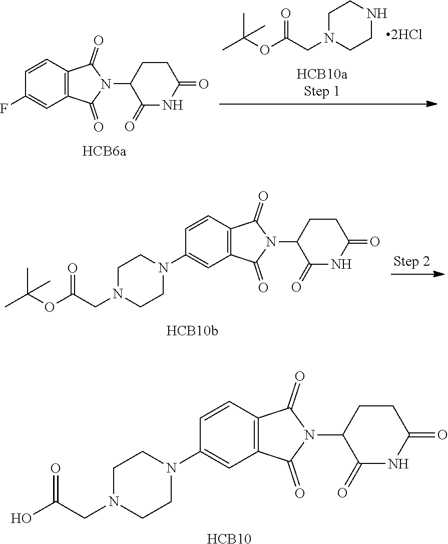

### Step 1: tert-Butyl 2-{4-[2-(2,6-dioxopiperidin-3-yl)-1,3-dioxo-2,3-dihydro-1H-isoindol-5-yl]piperazin-1-yl}acetate (HCB10b)

To a solution of tert-butyl piperazin-1-yl-acetate dihydrochloride (HCB10a) (4.46 g, 0.0163 mmol, 1.1 equiv) in DMSO (29.7 mL, 0.5 M) were added DIPEA (3.93 mL, 0.0297 mmol, 2 equiv) and 2-(2,6-dioxopiperidin-3-yl)-5-fluoroisoindoline-1,3-dione (HCB6a) (4.1 g, 0.0148 mmol, 1 equiv). The reaction mixture was heated to 90° C. under argon for 40 h. The reaction mixture was cooled down to rt and water (5 mL) was added dropwise. A bright-yellow precipitate was formed, filtered off, and washed water (2×) on the filter. The filtrate was extracted with DCM (2×). The combined DCM layers were concentrated in vacuo and the crude oil was combined with bright-yellow precipitate for purification. The crude was purified by flash column chromatography to give tert-butyl 2-{4-[2-(2,6-dioxopiperidin-3-yl)-1,3-dioxo-2,3-dihydro-1H-isoindol-5-yl]piperazin-1-yl}acetate (HCB10b) as a yellow solid (5.49 g, 81%). ESI [M+H]⁺=457.7. ¹H NMR (300 MHz, Chloroform-d) δ 8.07 (s, 1H), 7.72 (d, J=8.5 Hz, 1H), 7.31 (d, J=2.3 Hz, 1H), 7.08 (dd, J=8.6, 2.4 Hz, 1H), 4.96 (dd, J=12.2, 5.2 Hz, 1H), 3.55-3.45 (m, 4H), 3.21 (s, 2H), 2.98-2.81 (m, 2H), 2.81-2.72 (m, 5H), 2.24-2.09 (m, 1H), 1.50 (s, 9H).

### Step 2: 2-{4-[2-(2,6-dioxopiperidin-3-yl)-1,3-dioxo-2,3-dihydro-1H-isoindol-5-yl]piperazin-1-yl}acetic acid trifluoroacetate (HCB10.TFA)

To a solution of tert-butyl 2-{4-[2-(2,6-dioxopiperidin-3-yl)-1,3-dioxo-2,3-dihydro-1H-isoindol-5-yl]piperazin-1-yl}acetate (5.49 g, 12.03 mmol, 1 equiv) (HCB10b) in DCM (100 mL, 0.12 M) was added TFA (50.6 mL, 661 mmol, 55 equiv). The reaction mixture was stirred 16 h at rt and then concentrated under reduced pressure. The resulting bright-yellow sticky solid was sonicated with anhydrous diethyl ether (200 mL) and additionally stirred for one hour. The resulting precipitate was filtered, washed twice with anhydrous Et₂O, and dried under reduced pressure to give a bright-yellow solid (HCB10.TFA) (6.55 g, quant;). LCMS (254 nm): Rt=2.69 min, 98.59%, ESI [M+H]⁺=401.14. ¹H NMR (400 MHz, DMSO-d₆) δ 11.11 (s, 1H), 10.87 (br s, 1H), 7.77 (d, J=8.4 Hz, 1H), 7.48 (d, J=2.3 Hz, 1H), 7.34 (dd, J=8.6, 2.3 Hz, 1H), 6.27 (br s, 2H), 5.10 (dd, J=12.9, 5.4 Hz, 1H), 4.22 (s, 2H), 4.11 (br s, 2H), 3.45 (br s, 6H), 3.38 (dd, J=139.7, 7.0 Hz, 8H), 2.90 (ddd, J=17.4, 14.1, 5.5 Hz, 1H), 2.65-2.52 (m, 2H), 2.10-1.97 (m, 1H).

### Example 11. 1-(2-(2,6-dioxopiperidin-3-yl)-1,3-dioxoisoindolin-5-yl)azetidine-3-carboxylic acid (HCB11)

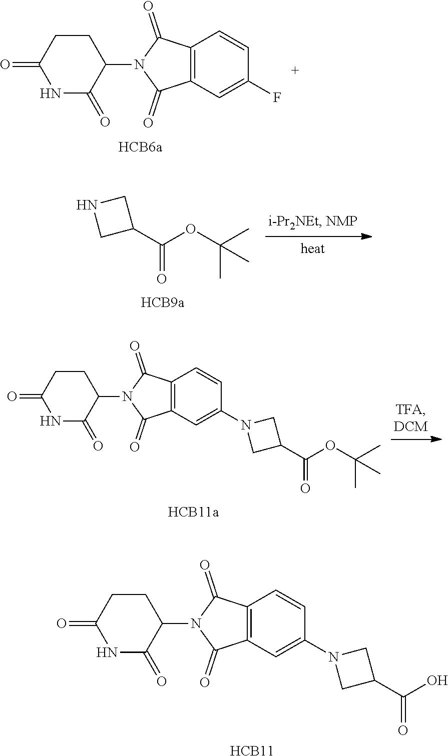

### Step 1: tert-butyl 1-(2-(2,6-dioxopiperidin-3-yl)-1,3-dioxoisoindolin-5-yl)azetidine-3-carboxylate (HCB11a)

A solution of HCB6a (200 mg, 0.72 mmol), HCB9a (114 mg, 0.72 mmol), and i-Pr₂NEt (441 μL, 2.53 mmol) in NMP (1.8 mL) was stirred at 90° C. overnight. The reaction mixture was diluted with H₂O and extracted with EtOAc. The combined organic layers were dried over Na₂SO₄ and concentrated. Flash chromatography (SiO₂, zero to 5% MeOH:DCM, gradient elution) provided the arylamine product (285 mg, 95%). LCMS: C₂₁H₂₃N₃O₆ requires: 413, found: m/z=414 [M+H]⁺.

### Step 2: 1-(2-(2,6-dioxopiperidin-3-yl)-1,3-dioxoisoindolin-5-yl)azetidine-3-carboxylic acid (HCB11)

TFA (496 μL, 6.48 mmol) was added to a solution of tert-butyl 1-(2-(2,6-dioxopiperidin-3-yl)-1,3-dioxoisoindolin-5-yl)azetidine-3-carboxylate (HCB11a) (134 mg, 0.32 mmol) in DCM (1.6 mL). After stirring for 2 h, the reaction mixture was concentrated under reduced pressure. Reverse phase flash chromatography (0-100% MeCN in H₂O) provided the desired carboxylic acid (30 mg, 0.08 mmol, 26%). LCMS: C₁₇H₁₅N₃O₆ requires: 357, found: m/z=358 [M+H]⁺.

### Example 12. 1-(2-(2,6-dioxopiperidin-3-yl)-1,3-dioxoisoindolin-5-yl)piperidine-4-carboxylic acid (HCB 12)

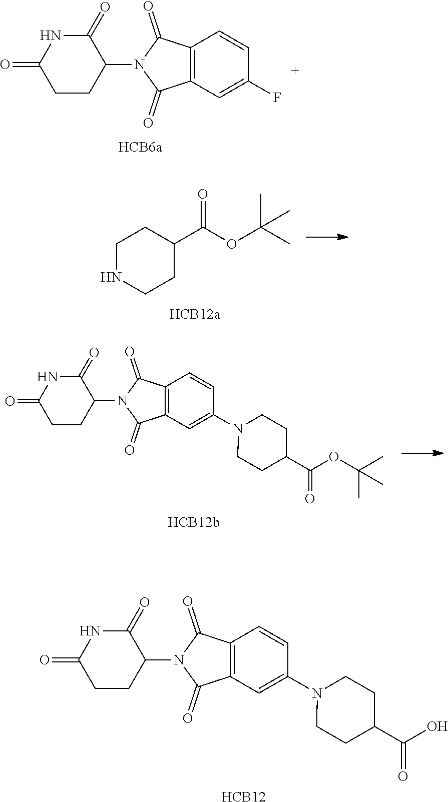

### Step 1: tert-butyl 1-(2-(2,6-dioxopiperidin-3-yl)-1,3-dioxoisoindolin-5-yl)piperidine-4-carboxylate (HCB 12b)

2-(2,6-dioxopiperidin-3-yl)-5-fluoroisoindole-1,3-dione (50 mg, 0.18 mmol) was treated with tert-butyl piperidine-4-carboxylate (34 mg, 0.18 mmol) to afford the desired product (107 mg, quant.). LCMS: C₂₃H₂₇N₃O₆ requires: 441, found: m/z=442 [M+H]⁺.

### Step 2: 1-(2-(2,6-dioxopiperidin-3-yl)-1,3-dioxoisoindolin-5-yl)piperidine-4-carboxylic acid (HCB 12)

Prepared in a similar manner as (HCB1) to give the title compound. LCMS C₁₉H₁₉N₃O₆ requires: 386, found: m/z=386 [M+H]⁺.

### Example 13. tert-butyl 1-(2-(2,6-dioxopiperidin-3-yl)-1,3-dioxoisoindolin-4-yl)piperidine-4-carboxylate (HCB 12′)

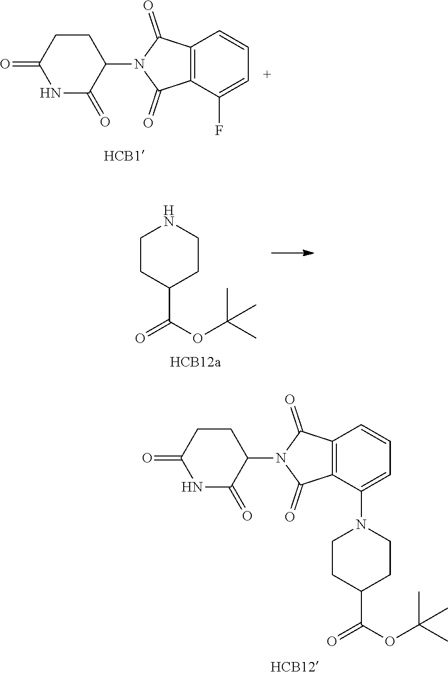 [SMILES: O=C1CCC(N2C(=O)c3cccc(F)c3C2=O)C(=O)N1 ; CC(C)(C)OC(=O)C1CCNCC1 ; CC(C)(C)OC(=O)C1CCN(c2cccc3c2C(=O)N(C2CCC(=O)NC2=O)C3=O)CC1]

Using the (HCB1) procedure, 2-(2,6-dioxopiperidin-3-yl)-4-fluoroisoindole-1,3-dione (HCB1′) (50 mg, 0.18 mmol) was treated with tert-butyl piperidine-4-carboxylate (HCB 12a) (34 mg, 0.18 mmol) to afford the desired product (133 mg, quant.). LCMS: C₂₃H₂₇N₃O₆ requires: 441, found: m/z=442 [M+H]⁺.

### Example 14. tert-butyl 1-(2-(2,6-dioxopiperidin-3-yl)-1,3-dioxoisoindolin-4-yl)azetidine-3-carboxylate (HCB 12″)

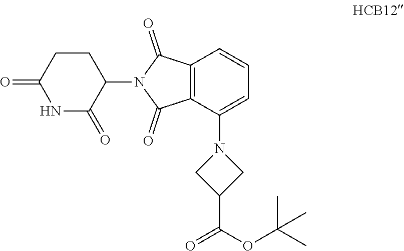 [SMILES: CC(C)(C)OC(=O)C1CN(c2cccc3c2C(=O)N(C2CCC(=O)NC2=O)C3=O)C1]

Using the (HCB1) procedure, 2-(2,6-dioxopiperidin-3-yl)-4-fluoroisoindole-1,3-dione (HCB1′) (200 mg, 0.72 mmol) was treated with tert-butyl azetidine-3-carboxylate (HCB9a) (28 mg, 0.18 mmol) to afford the desired product (152 mg, quant). LCMS: C₂₁H₂₃N₃O₆ requires: 413, found: m/z=414 [M+H]⁺.

### Example 15. 4-(4-(2-(2,6-dioxopiperidin-3-yl)-1,3-dioxoisoindolin-5-yl)piperazin-1-yl)butanoic acid (HCB13)

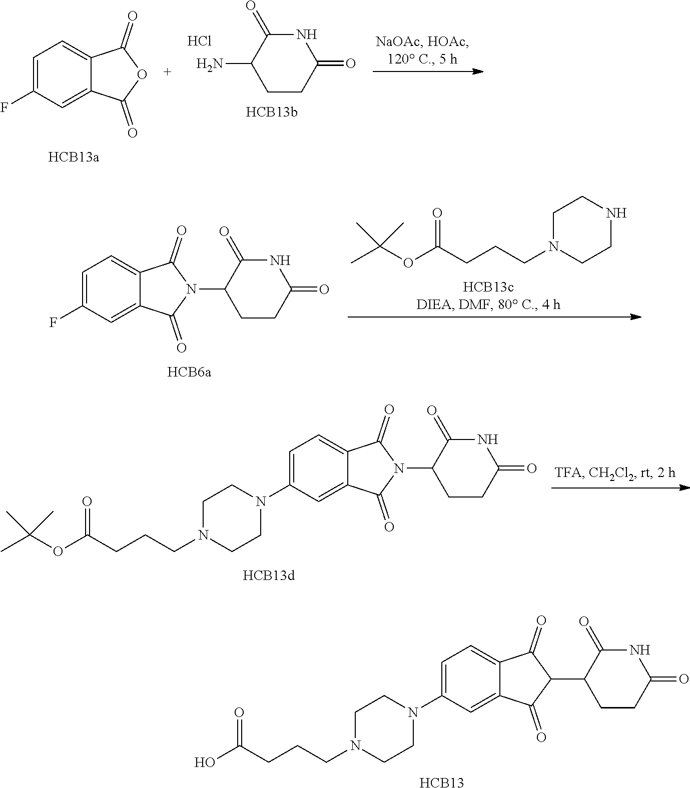

Step 1: To a solution of fluoro-benzofuran-1,3-dione (HCB13a) (27.16 mmol) in HOAc (50 mL) were added sodium acetate (46.17 mmol) and 3-aminopiperidine-2,6-dione hydrochloride (HCB13b) (38.02 mmol). The reaction mixture was stirred at 120° C. for 5 h. The mixture was cooled to room temperature and diluted with water. The solids were collected by filtration and dried to afford the fluoroimide intermediate 2-(2,6-dioxopiperidin-3-yl)-5-fluoroisoindoline-1,3-dione (3.0 g, 50%). LCMS: C₁₃H₉FN₂O₄ requires: 276, found: m/z=277 [M+H]⁺.

Step 2: To a solution of HCB6a (0.68 mmol) in DMF (30 mL) was added tert-butyl 4-(piperazin-1-yl)butanoate (HCB13c) (0.68 mmol) and N-ethyl-N-isopropylpropan-2-amine (1.4 mmol). The reaction mixture was stirred at 80° C. for 4 h. The resulting mixture was cooled to room temperature and diluted with water. The aqueous phase was extracted with ethyl acetate. The combined organic layer was washed with brine and water, dried over anhydrous sodium sulfate, filtered, and concentrated under vacuum to afford the tert-butyl ester intermediate (3.3 g, crude) which was used in the next step without further purification. Crude product tert-butyl 4-(4-(2-(2,6-dioxopiperidin-3-yl)-1,3-dioxoisoindolin-5-yl)piperazin-1-yl)butanoate (HCB13d) (4.4 g, 84%). LCMS: C₂₅H₃₂N₄O₆ requires: 484, found: m/z=485 [M+H]⁺.

Step 3: To a solution of the tert-butyl ester intermediate (6.57 mmol) in dichloromethane (20 mL) was added trifluoroacetic acid (10 mL). The reaction mixture was stirred at room temperature for 2 h, and then the solvent was removed under vacuum. The residue was purified by reverse phase flash column chromatography (20-80% acetonitrile in water) to afford the acid product 4-(4-(2-(2,6-dioxopiperidin-3-yl)-1,3-dioxoisoindolin-5-yl)piperazin-1-yl)butanoic acid (HCB13) TFA salt (3.35 g, 56%). LCMS: C₂₁H₂₄N₄O₆ requires: 428, found: m/z=429 [M+H]⁺.

### Synthesis of tert-butyl 4-(piperazin-1-yl)butanoate (HCB13c)

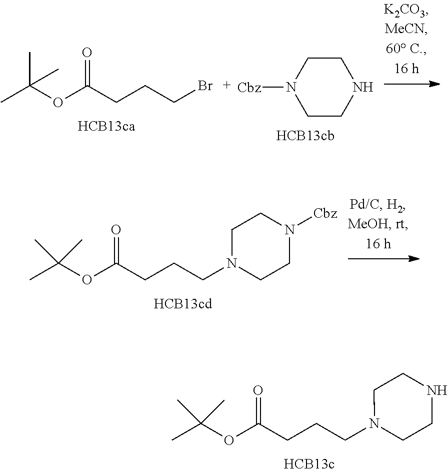 [SMILES: CC(C)(C)OC(=O)CCCBr ; O=C(OCc1ccccc1)N1CCNCC1 ; CC(C)(C)OC(=O)CCCN1CCN(C(=O)OCc2ccccc2)CC1 ; CC(C)(C)OC(=O)CCCN1CCNCC1]

Step 1: To a solution of tert-butyl 4-bromobutanoate (HCB13ca) (8.5 g, 38.10 mmol) and benzyl piperazine-1-carboxylate (HCB13cb) (10.1 g, 45.72 mmol) in acetonitrile (100 mL) was added K₂CO₃ (10.5 g, 76.20 mmol). The resulting mixture was stirred at 60° C. for 16 h under nitrogen atmosphere. The solids were filtered and the filtrate was concentrated under vacuum. The residue was purified by flash column chromatography with 10-70% ethyl acetate in petroleum ether to afford benzyl 4-(4-(tert-butoxy)-4-oxobutyl)piperazine-1-carboxylate (HCB13cd) (11.0 g, 79%) as colorless oil. LCMS: C₂₀H₃₀N₂O₄ requires: 362, found: m/z=363 [M+H]⁺.

Step 2: To a solution of benzyl 4-(4-(tert-butoxy)-4-oxobutyl)piperazine-1-carboxylate (HCB13cd) (11.0 g, 30.39 mmol) in methanol (150 mL) was added Pd/C (10%, 2 g) under nitrogen atmosphere. The mixture was stirred at room temperature for 16 h under hydrogen atmosphere (2 atm). The solids were filtered and the filtrate was concentrated under vacuum to afford tert-butyl 4-(piperazin-1-yl)butanoate (HCB13c) (6.6 g, crude) which was used in the next step without further purification. LCMS: C₁₂H₂₄N₂O₂ requires: 228, found: m/z=229 [M+H]⁺.

### Example 16. 6-((2-(2,6-dioxopiperidin-3-yl)-1,3-dioxoisoindolin-4-yl)amino)hexanoic acid (HCB14)

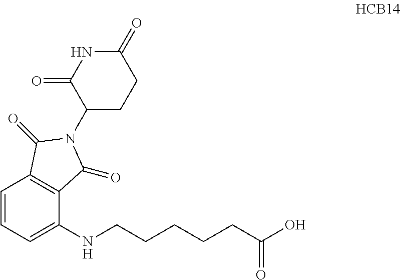 [SMILES: O=C(O)CCCCCNc1cccc2c1C(=O)N(C1CCC(=O)NC1=O)C2=O]

HCB14 was prepared in a similar manner as HCB3 by substituting 2-(2,6-dioxopiperidin-3-yl)-4-fluoroisoindoline-1,3-dione (HCB1a′) for 2-(2,6-dioxopiperidin-3-yl)-5-fluoroisoindoline-1,3-dione (HCB6a) to give the title compound. LCMS C₁₉H₂₁N₃O₆ requires: 387.1, found: m/z=385.9[M−H]⁻

### Example 17. 8-((2-(2,6-dioxopiperidin-3-yl)-1,3-dioxoisoindolin-5-yl)amino)octanoic acid (HCB15)

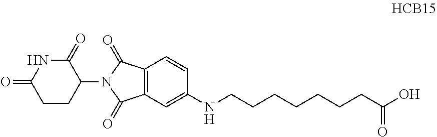 [SMILES: O=C(O)CCCCCCCNc1ccc2c(c1)C(=O)N(C1CCC(=O)NC1=O)C2=O]

HCB15 was prepared in a similar manner as HCB3 by substituting tert-butyl 6-aminohexanoate hydrochloride for tert-butyl 8-aminooctanoate to give the title compound. LCMS: C₂₁H₂₅N₃O₆ requires: 415.2, found: m/z=414.2[M−H]

### Example 18. 2-(2,6-dioxopiperidin-3-yl)-4-(piperazin-1-yl)isoindole-1,3-dione (HCB16)

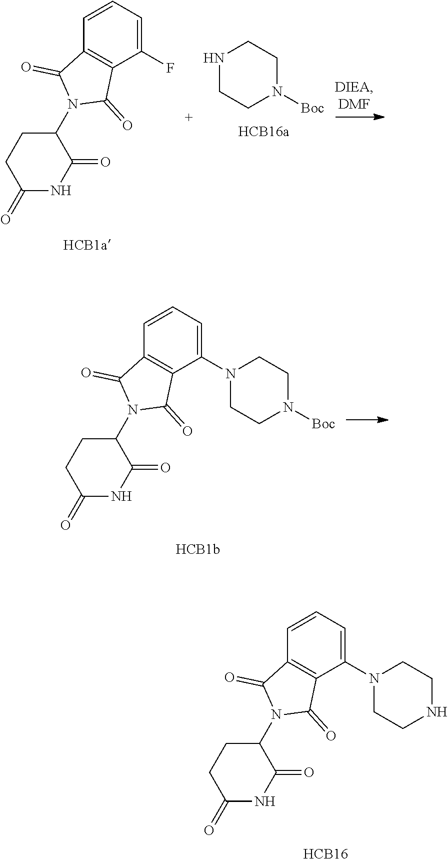

Step 1: A mixture of 2-(2,6-dioxopiperidin-3-yl)-4-fluoroisoindole-1,3-dione (497 mg, 1.8 mmol), tert-butyl piperazine-1-carboxylate (346 mg, 1.86 mmol), and i-Pr₂NEt (1 mL, 5.73 mmol) in DMF (6 mL) was allowed to stir at 90° C. overnight. The reaction was diluted with H₂O and extracted with EtOAc three times. The combined organic layers were dried over Na₂SO₄, filtered, concentrated, and purified by flash chromatography on a 24 g column, eluted by gradient elution with zero to 10% MeOH:CH₂Cl₂, to provide tert-butyl 4-[2-(2,6-dioxopiperidin-3-yl)-1,3-dioxoisoindol-4-yl]piperazine-1-carboxylate (0.536 g, 67.3%) as a yellow solid. LCMS: C₂₂H₂₆N₄O₆ requires: 442, found m/z=443 [M+H]⁺.

Step 2: A solution of 4 M hydrogen chloride (1.4 mL, 5.6 mmol) in dioxane was added dropwise to a stirring solution of tert-butyl 4-[2-(2,6-dioxopiperidin-3-yl)-1,3-dioxoisoindol-4-yl]piperazine-1-carboxylate (306 mg, 0.69 mmol) in methylene chloride (1.4 mL). The reaction stirred at room temperature for two hours. The volatiles were removed to provide 2-(2,6-dioxopiperidin-3-yl)-4-(piperazin-1-yl)isoindole-1,3-dione (0.2362 g, 99.8%) as a white solid. LCMS: C₁₇H₁₈N₄O₄ requires: 342.1, found m/z=343.3 [M+H]⁺.

### CRBN-Targeting LHP Generally Prepared According to Scheme B2

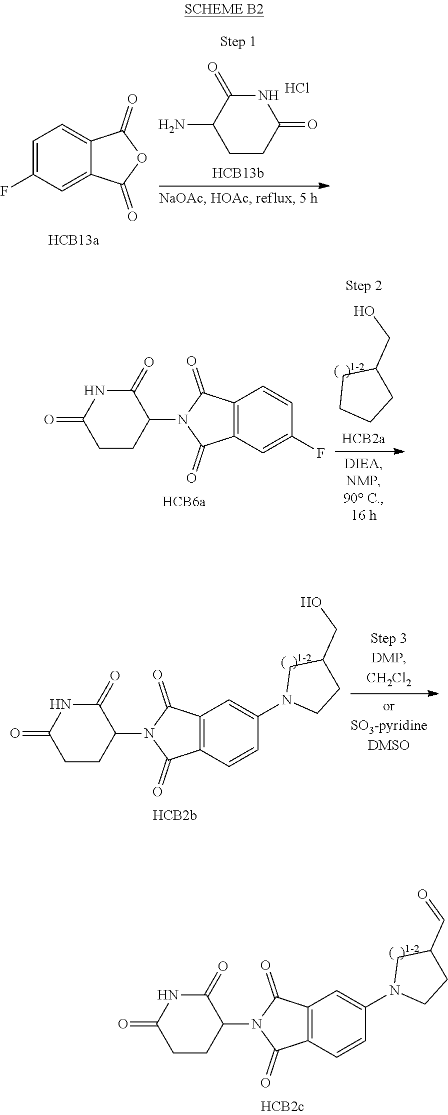

### Step 1: 2-(2,6-dioxopiperidin-3-yl)-5-fluoroisoindoline-1,3-dione (HCB6a)

A mixture of 5-fluoro-1,3-dihydro-2-benzofuran-1,3-dione (5.0 g, 30.10 mmol), 3-aminopiperidine-2,6-dione hydrochloride (6.9 g, 42.14 mmol), and NaOAc (4.2 g, 51.17 mmol) in HOAc (50 mL) was stirred at 120° C. for 5 h before concentration under vacuum. The residue was washed with water and the solid was collected by filtration. The crude product was washed with water twice, ethyl acetate twice, and dried in an oven to afford 2-(2,6-dioxopiperidin-3-yl)-5-fluoroisoindoline-1,3-dione (7.7 g, 92%) as a light brown solid. ¹H NMR (300 MHz, DMSO-d₆) δ 11.16 (s, 1H), 8.03-8.00 (m, 1H), 7.87-7.85 (m, 1H), 7.75-7.70 (m, 1H), 5.19-5.15 (m, 1H), 2.94-2.86 (m, 1H), 2.63-2.48 (m, 2H), 2.12-2.06 (m, 1H). ¹⁹F NMR (300 MHz, DMSO-d₆) δ-102.078.

### Step 2: Amine Displacement of Aryl Fluoride

To a solution of 2-(2,6-dioxopiperidin-3-yl)-5-fluoro-2,3-dihydro-1H-isoindole-1,3-dione (1.0 g, 3.62 mmol) in N-Methyl pyrrolidone (10 mL) were added the amine (3.60 mmol) and DIEA (1.4 g, 10.83 mmol). The resulting solution was stirred at 80° C. for 16 h. The reaction mixture was cooled down to room temperature and purified by reverse phase flash chromatography to afford the corresponding final product.

### Step 3: Alcohol Oxidation to the Aldehyde

To a mixture of the alcohol (1.06 mmol) in CH₂Cl₂ (10 mL) was added Dess-Martin periodinane (2.12 mmol). The mixture was allowed to stir at room temperature for one hour. The mixture was purified by column chromatography to afford the desired aldehyde.

Described below are additional examples of CRBN-targeting LHP building blocks prepared according to Scheme B2.

### Example 19. (3S)-1-(2-(2,6-dioxopiperidin-3-yl)-1,3-dioxoisoindolin-5-yl)pyrrolidine-3-carbaldehyde (HCB17)

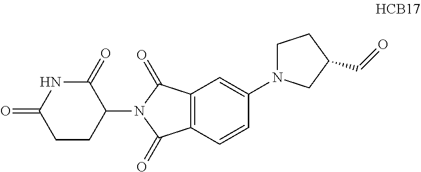 [SMILES: O=C[C@H]1CCN(c2ccc3c(c2)C(=O)N(C2CCC(=O)NC2=O)C3=O)C1]

### Step 1: 2-(2,6-dioxopiperidin-3-yl)-5-((S)-3-(hydroxymethyl)pyrrolidin-1-yl)isoindoline-1,3-dione

Following Step 1 of Scheme B2, 2-(2,6-dioxopiperidin-3-yl)-5-fluoroisoindoline-1,3-dione was treated with (S)-pyrrolidin-3-ylmethanol to afford 2-(2,6-dioxopiperidin-3-yl)-5-((S)-3-(hydroxymethyl)pyrrolidin-1-yl)isoindoline-1,3-dione (643.1 mg, 33%) as a yellow solid. ¹H NMR (300 MHz, DMSO-d₆) δ 11.08 (s, 1H), 7.64 (d, J 8.4 Hz, 1H), 6.89 (d, J 2.1 Hz, 1H), 6.80 (dd, J 8.4, 2.1 Hz, 1H), 5.06 (dd, J 12.9, 5.4 Hz, 1H), 4.78 (t, J 5.4 Hz, 1H), 3.59-3.41 (m, 5H), 3.22-3.17 (m, 1H), 2.95-2.83 (m, 1H), 2.67-2.44 (m, 3H), 2.12-1.88 (m, 2H), 1.87-1.76 (m, 1H). MS (ESI) calc'd for C₁₈H₁₉N₃O₅ [M+H]⁺: 358.1; found 358.1.

### Step 2: (3S)-1-(2-(2,6-dioxopiperidin-3-yl)-1,3-dioxoisoindolin-5-yl)pyrrolidine-3-carbaldehyde

To a mixture of 2-(2,6-dioxopiperidin-3-yl)-5-[(3S)-3-(hydroxymethyl)pyrrolidin-1-yl]isoindole-1,3-dione (258 mg, 0.72 mmol) in DCM (5 mL) was added 1,1-bis(acetyloxy)-3-oxo-1λ⁵,2-benziodaoxol-1-yl acetate (0.61 g, 1.44 mmol). After 90 minutes, silica gel was added and the mixture was concentrated to dryness. The resulting powder was transferred to a loading cartridge and the mixture was purified by flash chromatography on a 24 g column eluted with zero to 100% ethyl acetate:hexanes to provide (3S)-1-[2-(2,6-dioxopiperidin-3-yl)-1,3-dioxoisoindol-5-yl]pyrrolidine-3-carbaldehyde (198 mg, 77%). LCMS: C₁₈H₁₇N₃O₅ requires: 355, found: m/z=356 [M+H]⁺.

### Example 20. 1-(2-(2,6-dioxopiperidin-3-yl)-1,3-dioxoisoindolin-5-yl)piperidine-4-carbaldehyde (HCB18)

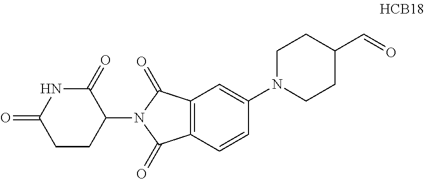 [SMILES: O=CC1CCN(c2ccc3c(c2)C(=O)N(C2CCC(=O)NC2=O)C3=O)CC1]

### Step 1: 2-(2,6-dioxopiperidin-3-yl)-5-(4-(hydroxymethyl)piperidin-1-yl)isoindoline-1,3-dione

Following Step 1 of Scheme B2, 2-(2,6-dioxopiperidin-3-yl)-5-fluoroisoindoline-1,3-dione was treated with piperidin-4-ylmethanol to afford 2-(2,6-dioxopiperidin-3-yl)-5-(4-(hydroxymethyl)piperidin-1-yl)isoindoline-1,3-dione (939 mg, 70%) as a yellow solid. ¹H NMR (300 MHz, DMSO-d₆) δ 11.09 (s, 1H), 7.65 (d, J 8.4 Hz, 1H), 7.30 (d, J 2.4 Hz, 1H), 7.23 (dd, J 8.4, 2.4 Hz, 1H), 5.07 (dd, J 12.6, 5.4 Hz, 1H), 4.51 (t, J 5.1 Hz, 1H), 4.07 (d, J 13.2 Hz, 2H), 3.27 (t, J=5.7 Hz, 2H), 2.99-2.80 (m, 3H), 2.62-2.55 (m, 2H), 2.17-1.95 (m, 1H), 1.76-1.67 (m, 3H), 1.24-1.12 (m, 2H). MS (ESI) calc'd for C₁₉H₂₁N₃O₅ [M+H]⁺: 372.1; found 372.2.

### Step 2: 1-(2-(2,6-dioxopiperidin-3-yl)-1,3-dioxoisoindolin-5-yl)piperidine-4-carbaldehyde

According to Scheme B2, 2-(2,6-dioxopiperidin-3-yl)-5-(4-(hydroxymethyl)piperidin-1-yl)isoindoline-1,3-dione was oxidized to afford 1-(2-(2,6-dioxopiperidin-3-yl)-1,3-dioxoisoindolin-5-yl)piperidine-4-carbaldehyde. LCMS: C₁₉H₁₉N₃O₅ requires: 369, found: m/z=370 [M+H]⁺.

### Example 21. (2S)-4-(2-(2,6-dioxopiperidin-3-yl)-1,3-dioxoisoindolin-5-yl)morpholine-2-carbaldehyde (HCB19)

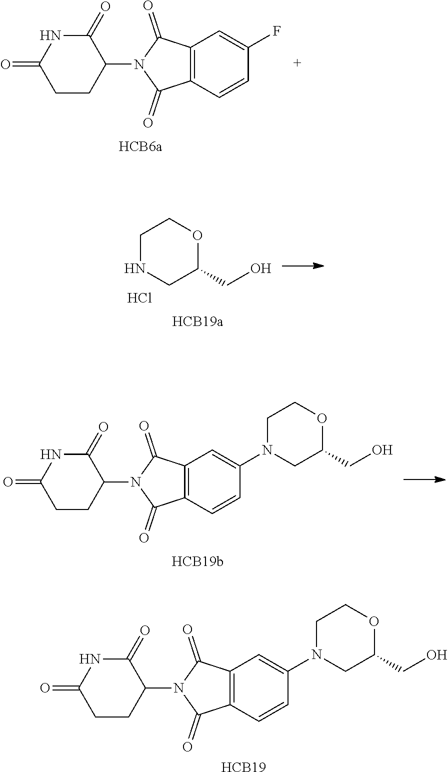

### Step 1: (2S)-4-(2-(2,6-dioxopiperidin-3-yl)-1,3-dioxoisoindolin-5-yl)morpholine-2-carbaldehyde

Following the Scheme B2 Step 2 procedure, (2S)-morpholin-2-ylmethanol hydrochloride (100 mg, 0.65 mmol) was treated with rac-2-[(3R)-2,6-dioxopiperidin-3-yl]-5-fluoroisoindole-1,3-dione (180 mg, 0.65 mmol). Reverse phase flash chromatography (zero to 50% MeCN:H₂O, gradient elution) provided the desired product (101 mg, 0.27 mmol, 42%). LCMS: C₁₈H₁₉N₃O₆ requires: 373, found: m/z=374 [M+H]⁺.

### Step 2: (2S)-4-(2-(2,6-dioxopiperidin-3-yl)-1,3-dioxoisoindolin-5-yl)morpholine-2-carbaldehyde

To a mixture of 2-[(3RS)-2,6-dioxopiperidin-3-yl]-5-[(2S)-2-(hydroxymethyl)morpholin-4-yl]isoindole-1,3-dione (20 mg, 0.05 mmol) in DMSO (0.2 mL) and DCM (0.2 mL) was added Et₃N (82 μL, 0.55 mmol) and SO₃-pyridine (40 mg, 0.25 mmol). The reaction mixture was allowed to stir at room temperature for two hours, and concentrated under a positive flow of N₂. Flash chromatography afforded the desired product (17 mg, 0.05 mmol, 86%). LCMS: C₁₈H₁₇N₃O₆ requires: 371, found: m/z=372 [M+H]⁺.

### Example 22. 1-{[(3R)-1-[2-(2,6-dioxopiperidin-3-yl)-1,3-dioxoisoindol-5-yl]pyrrolidin-3-yl]methyl}piperidine-4-carboxylic acid (HCB 20)

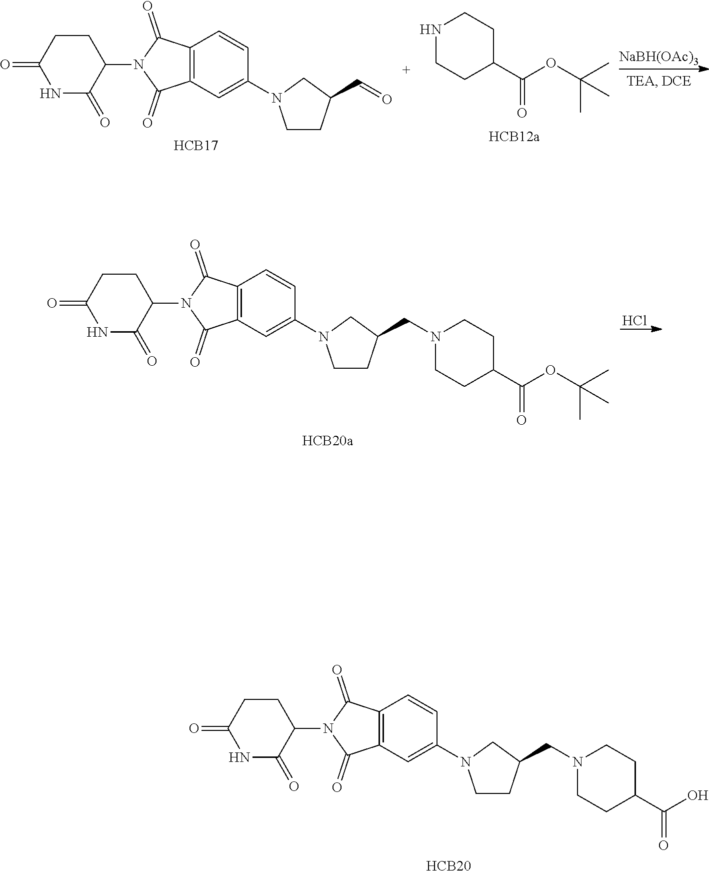

Step 1: To a solution of (3S)-1-[2-(2,6-dioxopiperidin-3-yl)-1,3-dioxoisoindol-5-yl]pyrrolidine-3-carbaldehyde (50 mg, 0.14 mmol) and tert-butyl piperidine-4-carboxylate (26 mg, 0.14 mmol) in DCE (1.4 mL) was added TEA (97.5 mL, 0.7 mmol, 5 equiv) and sodium triacetoxyborohydride (89 mg, 0.42 mmol, 3 equiv) and the reaction was stirred for one hour. The reaction was quenched with water, extracted with DCM, dried over Na₂SO₄, and concentrated under reduced pressure. Flash chromatography (SiO₂, 0-5% MeOH:DCM gradient elution) to afford the desired product (69 mg, 0.13 mmol, 94%). LCMS: C₂₈H₃₆N₄O₆ requires: 524, found: m/z=525 [M+H]⁺.

Step 2: tert-butyl 1-{[(3R)-1-[2-(2,6-dioxopiperidin-3-yl)-1,3-dioxoisoindol-5-yl]pyrrolidin-3-yl]methyl}piperidine-4-carboxylate (34 mg, 0.06 mmol) was dissolved in DCM (1 mL), followed by the addition of 2 M HCl in dioxane (1 mL, 2 mmol) and the reaction mixture was stirred for 45 min. Then the solvent was removed under reduced pressure. The crude product (44.7 mg, quant.) was used without purification. LCMS: C₂₄H₂₈N₄O₆ requires: 469, found: m/z=470 [M+H]⁺.

### Example 23. 2-(2,6-dioxopiperidin-3-yl)-4-((17-hydroxy-3,6,9,12,15-pentaoxaheptadecyl)amino)isoindoline-1,3-dione (HCB21)

### Synthesis of 2-(2-((2-(2,6-dioxopiperidin-3-yl)-1,3-dioxoisoindolin-4-yl)amino)ethoxy)ethyl 4-methylbenzenesulfonate (HCB21)

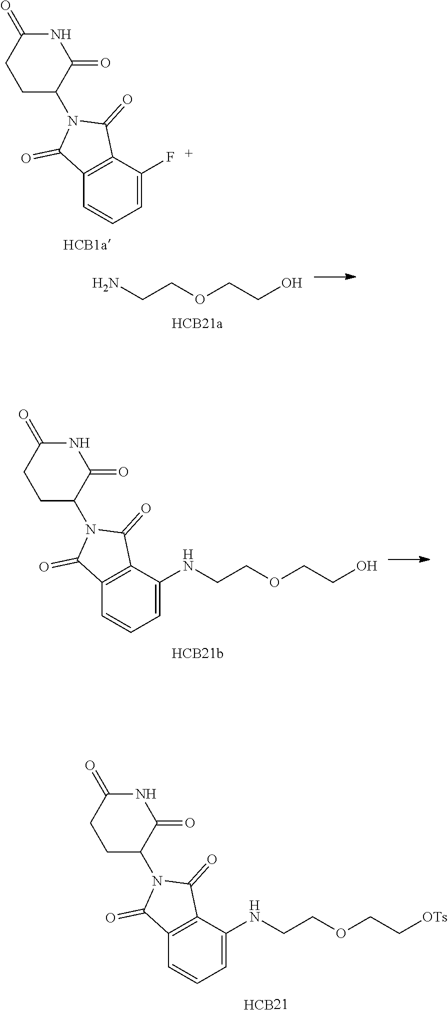 [SMILES: O=C1CCC(N2C(=O)c3cccc(F)c3C2=O)C(=O)N1 ; NCCOCCO ; O=C1CCC(N2C(=O)c3cccc(NCCOCCO)c3C2=O)C(=O)N1 ; Cc1ccc(S(=O)(=O)OCCOCCNc2cccc3c2C(=O)N(C2CCC(=O)NC2=O)C3=O)cc1]

Step 1: Using the general procedure described in Scheme B2 Step 2, 2-(2,6-dioxo-3-piperidyl)-4-fluoro-isoindoline-1,3-dione (4 g, 14.21 mmol) and 2-(2-aminoethoxy)ethanol (1.42 mL, 14.21 mmol) was substituted to provide 2-(2,6-dioxo-3-piperidyl)-4-[2-(2-hydroxyethoxy)ethylamino]isoindoline-1,3-dione (2.8 g, 7.07 mmol, 49.75%) as a yellow oil. LCMS: C₁₇H₁₉N₃O₆ requires: 361, found: m/z=362 [M+H]⁺.

Step 2: To a solution of 2-(2,6-dioxo-3-piperidyl)-4-[2-(2-hydroxyethoxy)ethylamino]isoindoline-1,3-dione (2.8 g, 7.07 mmol) and pyridine (9 mL, 111.5 mmol) in DCM (20 mL) was added TsCl (2.79 g, 14.63 mmol). After stirring at rt overnight, the reaction mixture was washed with saturated aqueous NaCl, dried over Na₂SO₄, and concentrated under reduced pressure. HPLC (H₂O:MeCN with 0.1% TFA) afforded the tosylated product. 2-(2,6-dioxo-3-piperidyl)-4-[2-[2-(2-hydroxyethoxy)ethoxy]ethylamino]isoindoline-1,3-dione was treated with TsCl as described above to provide 2-[2-[[2-(2,6-dioxo-3-piperidyl)-1,3-dioxo-isoindolin-4-yl]amino]ethoxy]ethyl 4-methylbenzenesulfonate (1.2 g, 2.39 mmol, 34%). LCMS: C₂₄H₂₅N₃O₈S requires: 515, found: m/z=516 [M+H]⁺.

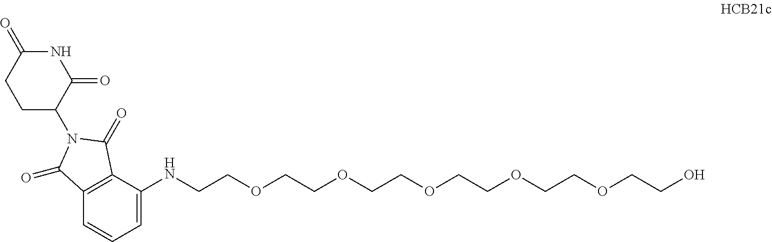 [SMILES: O=C1CCC(N2C(=O)c3cccc(NCCOCCOCCOCCOCCOCCO)c3C2=O)C(=O)N1]

Using Step 1 of Example 23, 2-(2,6-dioxo-3-piperidyl)-4-fluoro-isoindoline-1,3-dione (2 g, 7.10 mmol) was treated with 2-[2-[2-[2-[2-(2-aminoethoxy)ethoxy]ethoxy]ethoxy]ethoxy]ethanol (2 g, 7.10 mmol) to afford 2-(2,6-dioxo-3-piperidyl)-4-[2-[2-[2-[2-[2-(2-hydroxyethoxy)ethoxy]ethoxy]ethoxy]ethoxy]ethylamino]isoindoline-1,3-dione (2.2 g, 4.01 mmol, 56.41%) as a yellow oil. LCMS: C₂₅H₃₅N₃O₁₀ requires: 537, found: m/z=538 [M+H]⁺.

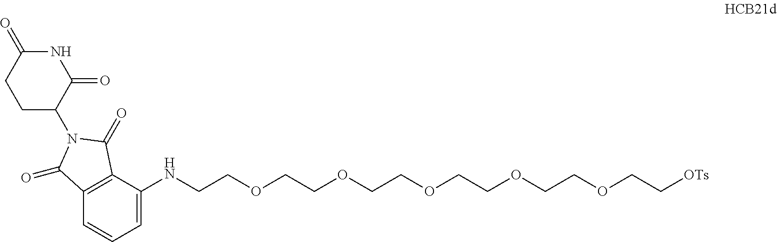 [SMILES: Cc1ccc(S(=O)(=O)OCCOCCOCCOCCOCCOCCNc2cccc3c2C(=O)N(C2CCC(=O)NC2=O)C3=O)cc1]

Using the general procedure in Step 2 of Example 23, 2-(2,6-dioxo-3-piperidyl)-4-[2-[2-[2-[2-[2-(2-hydroxyethoxy)ethoxy]ethoxy]ethoxy]ethoxy]ethylamino]isoindoline-1,3-dione (2.2 g, 4.01 mmol) was used to provide 2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl 4-methylbenzenesulfonate (0.507 g, 717.68 μmol, 17.91%) as a green oil. LCMS: C₃₂H₄₁N₃O₁₂S requires: 691, found: m/z=692 [M+H]⁺.

### Example 24. 3-(1-(2-(2,6-dioxopiperidin-3-yl)-1,3-dioxoisoindolin-5-yl)pyrrolidin-3-yl)propanoic acid (HCB51)

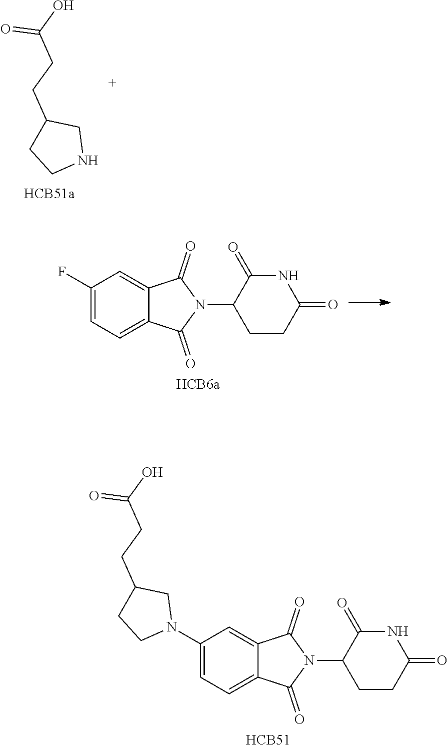

rac-3-[(3R)-pyrrolidin-3-yl]propanoic acid (100 mg, 0.70 mmol), rac-2-[(3R)-2,6-dioxopiperidin-3-yl]-5-fluoroisoindole-1,3-dione (193 mg, 0.70 mmol), and N,N-diisopropylethylamine (0.49 mL, 2.79 mmol) in DMF (2.00 mL) were heated at 90° C. for three hours. After cooling and sitting at room temperature for two days, the mixture was concentrated and then purified by flash chromatography on a 40 g column eluted with 0 to 10% MeOH:DCM

to provide 3-(1-(2-(2,6-dioxopiperidin-3-yl)-1,3-dioxoisoindolin-5-yl)pyrrolidin-3-yl)propanoic acid (0.0852 g, 30.5%). LCMS: C₂₀H₂₁N₃O₆ requires 399, found: m/z=400 [M+H]⁺.

### Example 25. 3-(6-(4-(2-hydroxyethyl)piperidin-1-yl)pyridin-3-yl)piperidine-2,6-dione (HCB54)

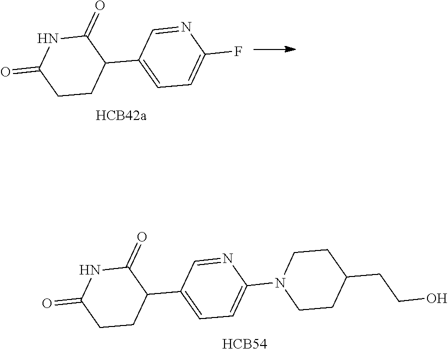 [SMILES: O=C1CCC(c2ccc(F)nc2)C(=O)N1 ; O=C1CCC(c2ccc(N3CCC(CCO)CC3)nc2)C(=O)N1 ; "HCB54"]

A mixture of 3-(6-fluoropyridin-3-yl)piperidine-2,6-dione (43.4 mg, 0.21 mmol), 4-piperidineethanol (26.9 mg, 0.21 mmol), and N,N-diisopropylethylamine (0.07 mL, 0.05 g, 0.42 mmol) in DMSO (1.00 mL) was heated in a 100° C. heating block for two days. Water was added and the mixture was extracted three times with ethyl acetate. The combined organic layers were concentrated. The crude residue was purified by preparative TLC in 5% MeOH:DCM to provide 3-(6-(4-(2-hydroxyethyl)piperidin-1-yl)pyridin-3-yl)piperidine-2,6-dione (0.0149 g, 22.5%). LCMS: C₁₇H₂₃N₃O₃ requires: 317, found: m/z=318 [M+H]⁺.

### General Schemes for Preparing LHP Building Blocks

CRBN-targeting LHL can be generally prepared according to Scheme C1:

### Scheme C1

### Synthesis of 3-(4-bromo-1-oxoisoindolin-2-yl)piperidine-2,6-dione (HCBC3)

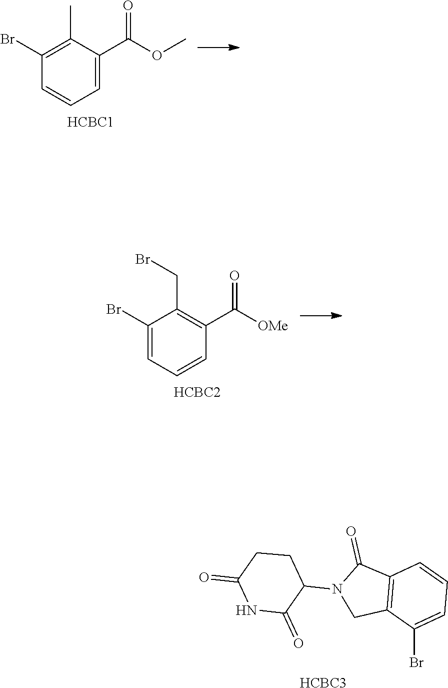

### Step 1: Synthesis of methyl 3-bromo-2-(bromomethyl)benzoate (HCBC2)

To a solution of methyl 3-bromo-2-methyl-benzoate (50 g, 218.27 mmol, 1 equiv) and NBS (46.62 g, 261.93 mmol, 1.2 equiv) in CHCl₃ (400 mL) was added AIBN (3.58 g, 21.83 mmol, 0.1 equiv). The mixture was stirred at 70° C. for 12 h, and then the reaction was concentrated under vacuum, diluted with DCM (400 mL), washed with H₂O (100 mL) and brine (100 mL), extracted with DCM (100 mL), and re-washed with brine (50 mL). The organic phase was combined, dried over Na₂SO₄, and concentrated in vacuum. The residue was purified by silica gel flash chromatography (Petroleum ether:Ethyl acetate=100:1) to yield 3-bromo-2-(bromomethyl)benzoate (63 g, 204.57 mmol, 93.72%) as a light yellow solid.

### Step 2: Synthesis of 3-(4-bromo-1-oxo-isoindolin-2-yl)piperidine-2,6-dione (HCBC3)

To a solution of methyl 3-bromo-2-(bromomethyl)benzoate (88.2 g, 286.39 mmol, 1 equiv) in MeCN (600 mL) was added DIEA (49.23 g, 380.91 mmol, 66.35 mL, 1.33 equiv) and 3-aminopiperidine-2,6-dione hydrochloride (51.01 g, 309.94 mmol, 1.08 equiv). The mixture was stirred at 80° C. for 16 hr. The reaction mixture was then filtered. and the filter cake was triturated by a mixture solution (EtOAc:H₂O=100 mL:200 mL) to yield 3-(4-bromo-1-oxo-isoindolin-2-yl)piperidine-2,6-dione (56.5 g, 174.85 mmol, 61.05%) as a purple powder.

### Example 26. 2-(4-(3-(2-(2,6-dioxopiperidin-3-yl)-1-oxoisoindolin-4-yl)propyl)piperazin-1-yl)acetic acid (HBC22)

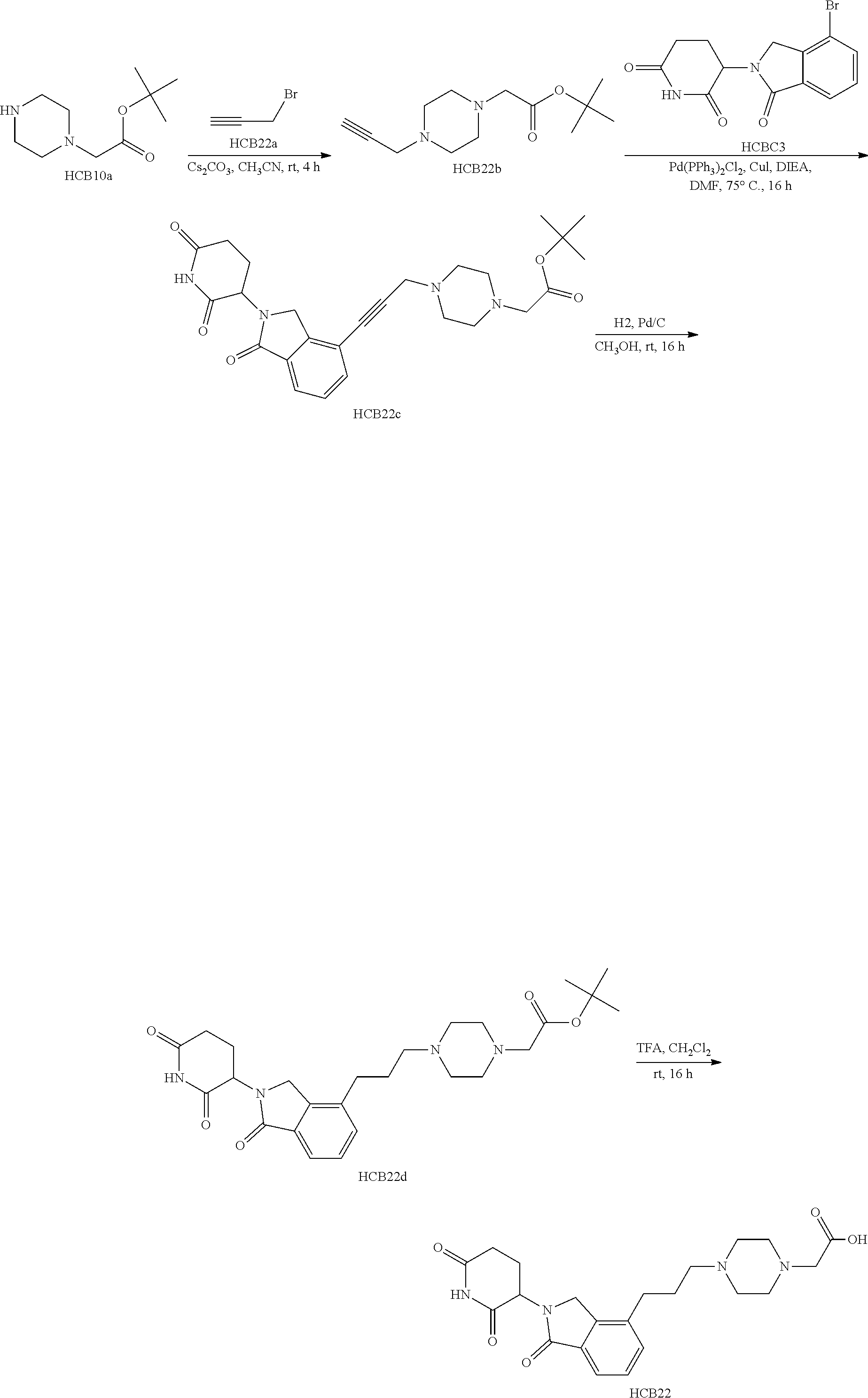

### Step 1: Synthesis of tert-butyl 2-(4-(prop-2-ynyl)piperazin-1-yl)acetate (HCB22b)

To a solution of tert-butyl 2-(piperazin-1-yl)acetate (1.5 g, 7.49 mmol) in acetonitrile (50 mL) were added 3-bromoprop-1-yne (892.5 mg, 7.50 mmol) and Cs₂CO₃ (2.4 g, 7.50 mmol). The resulting solution was stirred at room temperature for 4 h. The solids were filtered and the filtrate was evaporated under vacuum. The residue was purified by ISCO silica gel column with 0-30% ethyl acetate in petroleum ether to afford tert-butyl 2-(4-(prop-2-ynyl)piperazin-1-yl)acetate (1.1 g, 62%) as a yellow oil. MS (ESI) calculated for C₁₃H₂₂N₂O₂ [M+H]⁺: 239.2; found, 239.1.

### Step 2: Synthesis of tert-butyl 2-(4-(3-(2-(2,6-dioxopiperidin-3-yl)-1-oxoisoindolin-4-yl)prop-2-ynyl)piperazin-1-yl)acetate

To a degassed solution of 3-(4-bromo-1-oxoisoindolin-2-yl)piperidine-2,6-dione (1.5 g, 4.64 mmol) in N,N-dimethylformamide (30 mL) were added tert-butyl 2-(4-(prop-2-ynyl)piperazin-1-yl)acetate (1.5 g, 6.29 mmol), Pd(PPh₃)₂Cl₂ (489.0 mg, 0.70 mmol), DIEA (20 mL), and CuI (221.7 mg, 1.16 mmol). The resulting solution was stirred at 75° C. for 16 h under nitrogen. The reaction was quenched by the addition of water, and then extracted with ethyl acetate. The combined organic layer was washed with brine, dried over anhydrous sodium sulfate and filtered. The filtrate was evaporated under vacuum. The residue was purified by flash column chromatography with 0-10% methanol in dichloromethane to afford tert-butyl 2-(4-(3-(2-(2,6-dioxopiperidin-3-yl)-1-oxoisoindolin-4-yl)prop-2-ynyl)piperazin-1-yl)acetate (1.5 g, 68%) as a yellow solid. MS (ESI) calculated for C₂₆H₃₂N₄O₅ [M+H]⁺: 481.2; found, 481.1.

### Step 3: Synthesis of tert-butyl 2-(4-(3-(2-(2,6-dioxopiperidin-3-yl)-1-oxoisoindolin-4-yl)propyl)piperazin-1-yl)acetate (HCB22c)

To a solution of tert-butyl 2-(4-(3-(2-(2,6-dioxopiperidin-3-yl)-1-oxoisoindolin-4-yl)prop-2-ynyl)piperazin-1-yl)acetate (2.2 g, 4.58 mmol) in methanol (50 mL) was added Pd/C (dry, 0.44 g). The resulting solution was stirred at room temperature for 16 h under hydrogen (2 atm). The solids were filtered and the filtrate was evaporated under vacuum to afford tert-butyl 2-(4-(3-(2-(2,6-dioxopiperidin-3-yl)-1-oxoisoindolin-4-yl)propyl)piperazin-1-yl)acetate (1.4 g, crude) as a yellow oil, which was used in the next step without further purification. MS (ESI) calculated for C₂₆H₃₆N₄O₅ [M+H]⁺: 485.3; found, 485.2.

### Step 4: Synthesis of 2-(4-(3-(2-(2,6-dioxopiperidin-3-yl)-1-oxoisoindolin-4-yl)propyl)piperazin-1-yl)acetic acid TFA salt (HCB22c.TFA)

To a solution of tert-butyl 2-(4-(3-(2-(2,6-dioxopiperidin-3-yl)-1-oxoisoindolin-4-yl)propyl)piperazin-1-yl)acetate (1.4 g, 2.89 mmol) in dichloromethane (20 mL) was added trifluoroacetic acid (20 mL). The resulting solution was stirred at room temperature for 16 h before concentration under vacuum. The residue was purified by Prep-HPLC under the following conditions: [Column: XSelect CSH Prep C18 OBD Column, 5 Dim, 19*150 mm; Mobile Phase A: Water (0.05% TFA), Mobile Phase B: MeCN; Flow rate: 25 mL/min; Gradient: 5% B to 20% B in 7 min; 254/220 nm] to afford 2-(4-(3-(2-(2,6-dioxopiperidin-3-yl)-1-oxoisoindolin-4-yl)propyl)piperazin-1-yl)acetic acid TFA salt (434.3 mg, 35%) as a yellow solid. MS (ESI) calculated for C₂₂H₂₈N₄O₅ [M+H]⁺: 429.2; found, 429.0. ¹H NMR (300 MHz, DMSO-d₆) δ 11.08 (s, 1H), 7.60-7.65 (m, 1H), 7.52-7.47 (m, 2H), 5.20-5.13 (m, 1H), 4.52-4.46 (m, 1H), 4.35-4.29 (m, 1H), 3.51 (s, 3H), 3.47-2.84 (m, 9H), 2.72-2.50 (m, 4H), 2.49-2.31 (m, 1H), 2.05-1.97 (m, 3H).

### Example 27. (1R,3R)-3-(3-(2-(2,6-dioxopiperidin-3-yl)-1-oxoisoindolin-4-yl)propoxy)cyclobutane-1-carboxylic acid (HCB23)

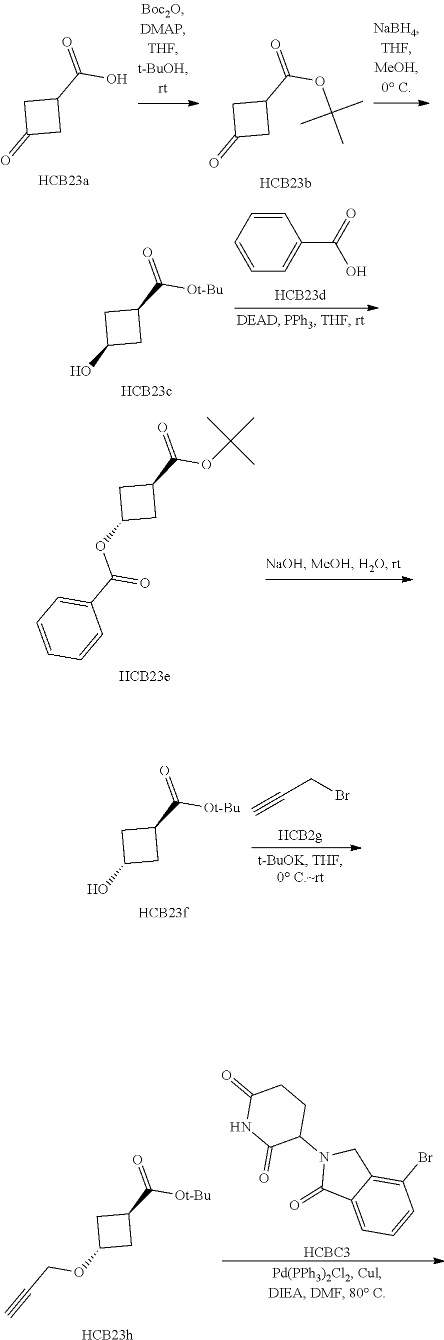

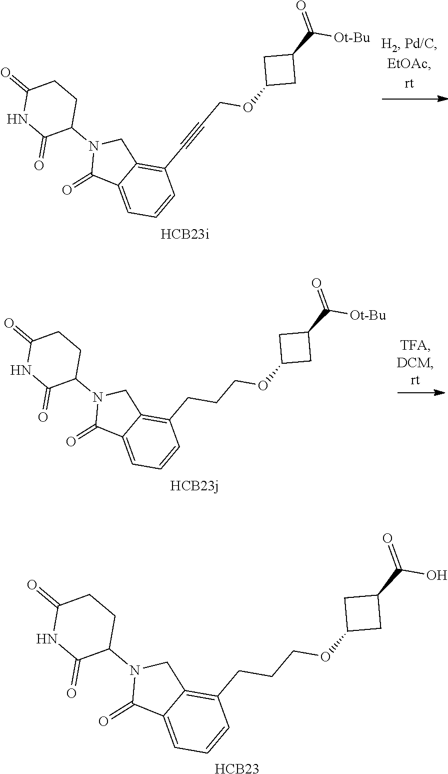

### Step 1: Synthesis of tert-butyl 3-oxocyclobutane-1-carboxylate (HCB23a)

To a solution of 3-oxocyclobutane-1-carboxylic acid (35.0 g, 306.7 mmol) in tetrahydrofuran (350 mL) and 2-methylpropan-2-ol (350 mL) was added N,N-dimethylpyridin-4-amine (100.4 g, 460.1 mmol) and Boc₂O (49.8 g, 228.6 mmol) at room temperature. After stirring for 5 h at room temperature under nitrogen, the reaction mixture was poured into water and extracted with ethyl acetate. The organic layers were dried over anhydrous sodium sulfate, filtered, and concentrated under vacuum. The residue was purified by flash column chromatography with 0-20% ethyl acetate in petroleum ether to afford tert-butyl 3-oxocyclobutane-1-carboxylate (17.0 g, 23%) as a yellow oil. ¹H NMR (300 MHz, DMSO-d₆) δ 3.37-3.11 (m, 5H), 1.44 (s, 9H).

### Step 2: Synthesis of cis-tert-butyl-3-hydroxycyclobutane-1-carboxylate (HCB23b)

To a solution of tert-butyl 3-oxocyclobutane-1-carboxylate (17.0 g, 100.0 mmol) in tetrahydrofuran (170 mL) and methanol (20 mL) was added sodium borohydride (1.9 g, 50.00 mmol) in portions at 0° C. After stirring for 15 min at 0° C., the reaction mixture was concentrated under vacuum. The residue was diluted with water and extracted with ethyl acetate three times. The organic layers were dried over anhydrous sodium sulfate, filtered, and concentrated under vacuum to afford cis-tert-butyl-3-hydroxycyclobutane-1-carboxylate (18.5 g, crude) as a yellow oil, which was used in the next step without further purification. ¹H NMR (300 MHz, Chloroform-d) δ 4.20-4.03 (m, 1H), 2.63-2.39 (m, 4H), 2.18-1.97 (m, 2H), 1.43 (s, 9H).

### Step 3: Synthesis of Trans-3-(Tert-Butoxycarbonyl)Cyclobutyl Benzoate (HCB23e)

To a solution of cis-tert-butyl-3-hydroxycyclobutane-1-carboxylate (12.0 g, 69.77 mmol), benzoic acid (17.5 g, 143.7 mmol), and triphenylphosphine (35.6 g, 136.0 mmol) in tetrahydrofuran (100 mL) was added DEAD (25.0 g, 143.7 mmol) dropwise at room temperature. After stirring for 16 h at room temperature, the reaction mixture was quenched by the addition of water, and then extracted with ethyl acetate three times. The organic layers were dried over anhydrous sodium sulfate, filtered, and concentrated under vacuum. The residue was purified by flash column chromatography with 0-20% ethyl acetate in petroleum ether to afford trans-3-(tert-butoxycarbonyl)cyclobutyl benzoate (14.3 g, 74%) as a yellow oil. ¹H NMR (300 MHz, DMSO-d₆) δ 8.03-7.92 (m, 2H), 7.74-7.61 (m, 1H), 7.61-7.48 (m, 2H), 5.34-5.19 (m, 1H), 3.18-2.99 (m, 1H), 2.66-2.34 (m, 4H), 1.44 (s, 9H).

### Step 4: Synthesis of trans-tert-butyl (1r,3r)-3-hydroxycyclobutane-1-carboxylate (HCB23f)

To a solution of trans-3-(tert-butoxycarbonyl)cyclobutyl benzoate (14.3 g, 51.81 mmol) in methanol (150 mL) and water (75 mL) was added sodium hydroxide (2.3 g, 57.5 mmol) at room temperature. After stirring for 16 h at room temperature, the reaction mixture was quenched by the addition of water and extracted with ethyl acetate three times. The organic layers were dried over anhydrous sodium sulfate, filtered, and concentrated under vacuum. The residue was purified by flash column chromatography with 0˜20% ethyl acetate in petroleum ether to afford trans-tert-butyl-3-hydroxycyclobutane-1-carboxylate (6.3 g, 71%) as a light yellow solid. ¹H NMR (300 MHz, Chloroform-d) δ 4.61-4.41 (m, 1H), 2.97-2.79 (m, 1H), 2.54-2.44 (m, 2H), 2.24-2.07 (m, 2H), 1.43 (s, 9H).

### Step 5: Synthesis of trans-tert-butyl-3-(prop-2-yn-1-yloxy)cyclobutane-1-carboxylate (HCB23 h)

To a solution of trans-tert-butyl-3-hydroxycyclobutane-1-carboxylate (6.0 g, 34.88 mmol) and 3-bromoprop-1-yne (6.2 g, 52.32 mmol) in tetrahydrofuran (30 mL) was added t-BuOK (53 mL, 1 M in THF) dropwise at 0° C. The mixture was stirred at room temperature for 48 h before concentration under vacuum. The residue was diluted with water and extracted with ethyl acetate three times. The organic layers were dried over anhydrous sodium sulfate, filtered, and concentrated under vacuum. The residue was purified by flash column chromatography with 0˜50% ethyl acetate in petroleum ether to afford trans-tert-butyl-3-(prop-2-yn-1-yloxy)cyclobutane-1-carboxylate (6.0 g, 82%) as a yellow oil. ¹H NMR (300 MHz, Chloroform-d) δ 4.42-4.29 (m, 1H), 4.14-4.01 (m, 2H), 3.03-2.87 (m, 1H), 2.56-2.45 (m, 2H), 2.42 (t, J=2.4 Hz, 1H), 2.37-2.12 (m, 2H), 1.45 (s, 9H).

### Step 6: Synthesis of trans-tert-butyl-3-((3-(2-(2,6-dioxopiperidin-3-yl)-1-oxoisoindolin-4-yl)prop-2-yn-1-yl)oxy)cyclobutane-1-carboxylate (HCB23i)

To a degassed solution of 3-(4-bromo-1-oxoisoindolin-2-yl)piperidine-2,6-dione (6.0 g, 18.58 mmol) in anhydrous DMF (100 mL) were added [1,1′-Bis(diphenylphosphino)ferrocene]dichloropalladium(II) dichloromethane adduct (2.0 g, 2.79 mmol), copper(I) iodide (0.9 g, 4.64 mmol), N,N-diisopropylethylamine (70 mL), and trans-tert-butyl-3-(prop-2-yn-1-yloxy)cyclobutane-1-carboxylate (5.9 g, 27.86 mmol) at room temperature. After stirring for 16 h at 80° C., the reaction mixture was poured into water and extracted with ethyl acetate. The organic layers were dried over anhydrous sodium sulfate, filtered, and concentrated under vacuum. The residue was purified by reversed phase flash column chromatography with 15-100% acetonitrile in water to afford trans-tert-butyl-3-((3-(2-(2,6-dioxopiperidin-3-yl)-1-oxoisoindolin-4-yl)prop-2-yn-1-yl)oxy)cyclobutane-1-carboxylate (1.6 g, 19%) as a pink solid. MS (ESI) calculated for C₂₅H₂₈N₂O₆ [M+H]⁺: 453.2; found: 453.2.

### Step 7: Synthesis of trans-tert-butyl-3-(3-(2-(2,6-dioxopiperidin-3-yl)-1-oxoisoindolin-4-yl)propoxy)cyclobu-tane-1-carboxylate (HCB23j)

A mixture of trans-tert-butyl-3-((3-(2-(2,6-dioxopiperidin-3-yl)-1-oxoisoindolin-4-yl)prop-2-yn-1-yl)oxy)cyclobutane-1-carboxylate (1.4 g, 3.10 mmol) and Pd/C (0.3 g, 10%) in ethyl acetate (50 mL) was stirred at room temperature for 16 h under H₂ atmosphere (2 atm). The solids were filtered and the filtrate was concentrated under vacuum to afford trans-tert-butyl-3-(3-(2-(2,6-dioxopiperidin-3-yl)-1-oxoisoindolin-4-yl)propoxy)cyclobu-tane-1-carboxylate (1.1 g, crude) as a brown solid, which was used in the next step without further purification. MS (ESI) calculated for C₂₅H₃₂N₂O₆ [M+H]⁺: 457.2; found: 457.2.

### Step 8: Synthesis of trans-3-(3-(2-(2,6-dioxopiperidin-3-yl)-1-oxoisoindolin-4-yl)propoxy)cyclobutane-1-carboxylic acid (HCB23)

To a solution of trans-3-(3-(2-(2,6-dioxopiperidin-3-yl)-1-oxoisoindolin-4-yl)propoxy)cyclobutane-1-carboxylate (1.1 g, 2.41 mmol) in dichloromethane (10 mL) was added trifluoroacetic acid (10 mL) at room temperature. After stirring at room temperature for 16 h, the reaction mixture was concentrated under vacuum. The residue was purified by reversed phase flash column chromatography with 15˜50% acetonitrile in water to afford trans-3-(3-(2-(2,6-dioxopiperidin-3-yl)-1-oxoisoindolin-4-yl)propoxy)cyclobutane-1-carboxylic acid (460.3 mg, 48%) as a white solid, which contained ˜60% cis-isomer by ¹H NMR. MS (ESI) calculated for C₂₁H₂₄N₂O₆ [M+H]⁺: 401.2; found: 401.1. ¹H NMR (400 MHz, DMSO-d₆) δ 12.17 (br s, 1H), 11.00 (s, 1H), 7.63-7.54 (m, 1H), 7.51-7.42 (m, 2H), 5.22-5.06 (m, 1H), 4.47 (d, J=17.2 Hz, 1H), 4.37-4.25 (m, 1H), 4.10-3.75 (m, 1H), 3.33-3.23 (m, 2H), 3.03-2.53 (m, 5H), 2.48-2.29 (m, 3H), 2.18-1.75 (m, 5H).

### Example 28. 3-(4-(5-hydroxypentyl)-1-oxoisoindolin-2-yl)piperidine-2,6-dione (HCB24)

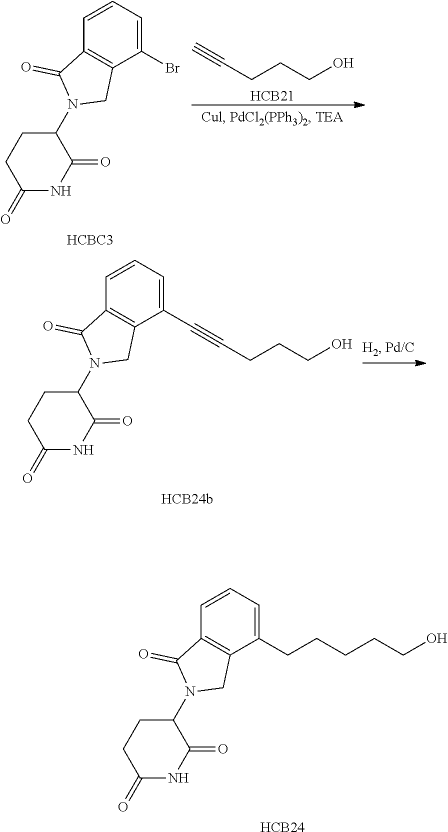

Step 1: A suspension of 3-(4-bromo-1-oxoisoindolin-2-yl)piperidine-2,6-dione (50 mg, 0.15 mmol), CuI (3.0 mg, 0.02 mmol), PdCl₂(PPh₃)₂(6.5 mg, 0.01 mmol), 4-pentyn-1-ol (20 mg, 0.23 mmol), and Et₃N (290 μL, 1.9 mmol) in DMF (0.75 mL) was flushed with N₂, and stirred at 90° C. for 16 h. The reaction mixture was cooled to rt, diluted with EtOAc and MeOH, and filtered through a pad of Celite. Flash chromatography (SiO₂, 0-10% MeOH:DCM gradient elution) afforded the desired product. LCMS: C₁₈H₁₈N₂O₄ requires: 326, found: m/z=349 [M+Na]⁺.

Step2: A solution of 3-(4-(5-hydroxypent-1-yn-1-yl)-1-oxoisoindolin-2-yl)piperidine-2,6-dione (110 mg, 0.34 mmol) in i-PrOH (5 mL) and DCM (2 mL) was stirred under H₂ in the presence of Pd/C (10 wt. %, 11 mg) for 16 h. The reaction mixture was filtered through Celite, and concentrated under reduced pressure to afford the desired product (100 mg, 89%). LCMS: C₁₈H₂₂N₂O₄ requires: 330, found: m/z=331 [M+H]⁺.

### Example 29. 5-(2-(2,6-dioxopiperidin-3-yl)-1-oxoisoindolin-4-yl)pentanoic acid (HCB 25)

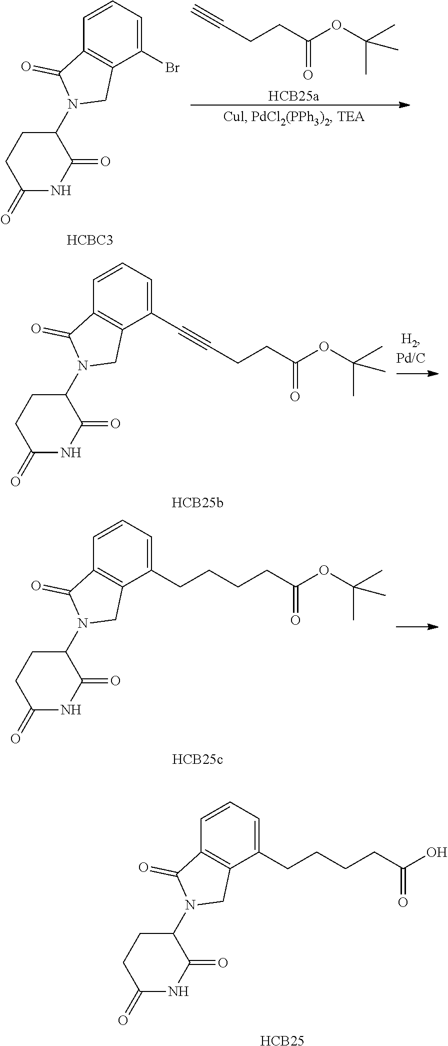

### Step 1 and Step 2: Used a similar procedure as Example 28, HBC24

Step 3: A solution of benzyl 5-[2-(2,6-dioxopiperidin-3-yl)-1-oxo-3H-isoindol-4-yl]pent-4-ynoate (81 mg, 0.19 mmol) in EtOAc (5 mL) and DCM (3 mL) was stirred under H₂ in the presence of Pd/C (10 wt. %, 8 mg) for 16 h. The reaction mixture was filtered through Celite and concentrated under reduced pressure to afford the desired product (60 mg, 93%). LCMS: C₁₈H₂₀N₂O₅ requires: 344, found: m/z=367 [M+Na]⁺.

### Example 30. 3-(3-(2-(2,6-dioxopiperidin-3-yl)-1-oxoisoindolin-4-yl)propoxy)propanoic acid (HCB 26)

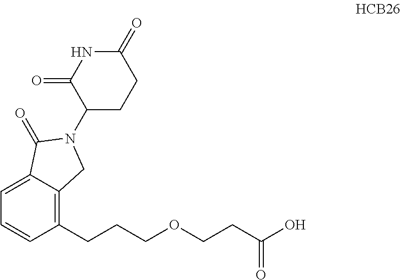 [SMILES: O=C(O)CCOCCCc1cccc2c1CN(C1CCC(=O)NC1=O)C2=O]

The synthetic procedures were similar to the HBC24 procedure.

LCMS: C₁₉H₂₂N₂O₆ requires: 374, found: m/z=375 [M+H]⁺.

### Example 31. 1-(2-(2,6-dioxopiperidin-3-yl)-1-oxoisoindolin-5-yl)piperidine-4-carbaldehyde (HCB27)

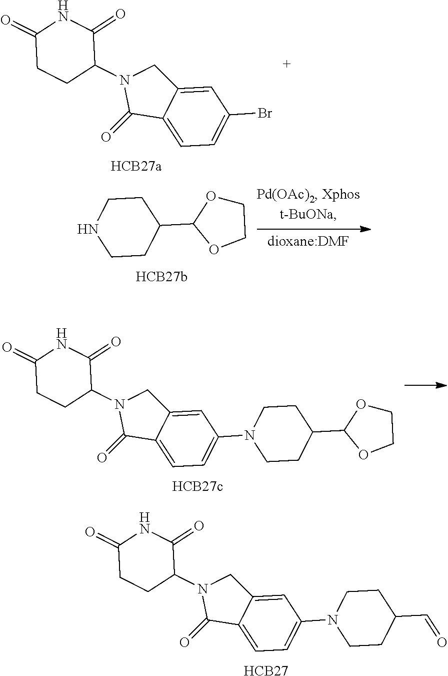

### Step 1: 3-{5-[4-(1,3-dioxolan-2-yl)piperidin-1-yl]-1-oxo-2,3-dihydro-1H-isoindol-2-yl}piperidine-2,6-dione (HCB27c)

A flask was charged with 4-(1,3-dioxolan-2-yl)piperidine (4.4 g, 28.22 mmol, 1.2 equiv), 3-(5-bromo-1-oxo-2,3-dihydro-1H-isoindol-2-yl)piperidine-2,6-dione (7.6 g, 23.52 mmol, 1 equiv), solid sodium tert-butoxide (6.7 g, 70.55 mmol, 3 equiv), and degassed dioxane (150 mL) with DMF (50 mL, 0.55 M). The flask was evacuated and backfilled with argon three times. XPhos (2.3 g, 4.7 mmol, 0.2 equiv) and Pd(OAc)₂ (0.79 mg, 3.5 mmol, 0.15 equiv) were added and argon was bubbled through the mixture for 25 min. The reaction mixture was monitored by LCMS and stirred at 100° C. overnight. Afterward, all volatiles were evaporated at reduced pressure and the resulting residue was purified via flash chromatography (20 to 70% EtOAc in hexane) to give the title product (0.7 g, 7%). ESI(+) [M+H]⁺=400.25. ¹H NMR (300 MHz, DMSO-d₆), δ 10.94 (s, 1H), 7.50 (d, J=8.9 Hz, 1H), 7.05 (m, 2H), 5.12-4.97 (m, 1H), 4.60 (d, J=4.6 Hz, 1H), 4.40-4.12 (m, 2H), 4.00-3.72 (m, 6H), 3.00-2.70 (m, 3H), 2.64-2.54 (m, 1H), 2.33 (m, 1H), 1.96 m, 1H), 1.82-1.67 (m, 3H), 1.47-1.28 (m, 2H).

### Step 2: 1-(2-(2,6-dioxopiperidin-3-yl)-1-oxoisoindolin-5-yl)piperidine-4-carbaldehyde (HCB27)

To a suspension of 3-{5-[4-(1,3-dioxolan-2-yl)piperidin-1-yl]-1-oxo-2,3-dihydro-1H-isoindol-2-yl}piperidine-2,6-dione (0.244 g, 0.6 mmol, 1 equiv) in THF:Water (4:1, 5 mL, 0.13 M) was added PPTS (0.31 g, 1.2 mmol, 2 equiv). The reaction mixture was heated to 70° C. for two days under argon. The volatiles were removed at reduced pressure and crude was purified by flash chromatography (zero to 20% acetone in DCM) to provide the title compound as a white crystalline solid (0.153 g, 68%). ¹H NMR (300 MHz, DMSO-d₆) δ 10.94 (s, 1H), 9.63 (s, 1H), 7.51 (d, J=8.5 Hz, 1H), 7.06 (m, 2H), 5.07 (m, 1H), 4.37-4.15 (m, 2H), 3.80 (m, 2H), 3.09-2.83 (m, 3H), 2.59 (m, 3H), 2.37 (m, 1H), 1.93 (m, 2H), 1.59 (m, 2H). LCMS: 205 nm, Rt=2.93 min, 90.66% purity; ESI(+) [M+H]⁺=357.23.

### Example 32. N-(2,6-dioxopiperidin-3-yl)-5-(4-formylpiperidin-1-yl)pyridine-2-carboxamide (HCB28)

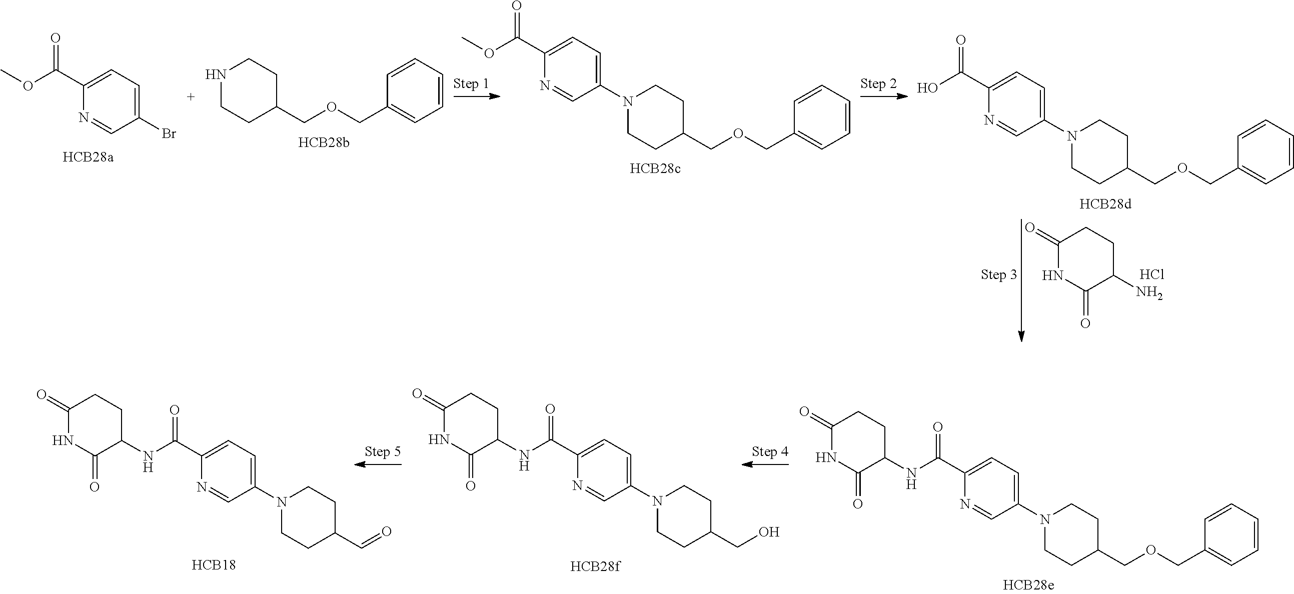

### Step 1: methyl 5-{4-[(benzyloxy)methyl]piperidin-1-yl}pyridine-2-carboxylate (HCB28c)

A mixture of methyl 5-bromopyridine-2-carboxylate (1.0 g, 4.63 mmol, 1.0 equiv), 4-[(benzyloxy)methyl]piperidine (950 mg, 4.63 mmol, 1.0 equiv), rac-BINAP (288 mg, 463 μmol, 0.1 equiv), Pd₂(dba)₃ (432 mg, 463 μmol, 0.1 equiv), and Cs₂CO₃ (4.52 g, 13.9 mmol, 3.0 equiv) was suspended in toluene (30 mL) and the mixture was heated to 100° C. for 12 h. The reaction mixture was cooled to rt and diluted with EtOAc (100 mL) before being filtered and purified (SiO₂, 10→100% EtOAc:Hexanes, product elutes at 70%) afforded methyl 5-{4-[(benzyloxy)methyl]piperidin-1-yl}pyridine-2-carboxylate (1.1 g, 67%). LCMS: C₂₀H₂₄N₂O₃ requires: 340, found: m/z=341 [M+H]⁺.

### Step 2: 5-{4-[(benzyloxy)methyl]piperidin-1-yl}pyridine-2-carboxylic acid (HCB28d)

Methyl 5-{4-[(benzyloxy)methyl]piperidin-1-yl}pyridine-2-carboxylate (510 mg, 1.50 mmol, 1.0 equiv) was suspended in MeOH:H₂O (1:4) and NaOH (90 mg, 2.25 mmol, 1.5 equiv) was added in one portion at rt. After 16 h, aq. HCl (1 M) was added to adjust the pH to five. The solids were collected by filtration to afford 5-{4-[(benzyloxy)methyl]piperidin-1-yl}pyridine-2-carboxylic acid (800 mg, 83%). LCMS: C₁₉H₂₂N₂O₃ requires: 326, found: m/z=327 [M+H]⁺.

### Step 3: 5-{4-[(benzyloxy)methyl]piperidin-1-yl}-N-(2,6-dioxopiperidin-3-yl)pyridine-2-carboxamide (HCB28e)

To a solution of 5-{4-[(benzyloxy)methyl]piperidin-1-yl}pyridine-2-carboxylic acid (510 mg, 1.56 mmol, 1.0 equiv) in DMF (1 mL) was added HATU (594 mg, 1.56 mmol, 1.0 equiv) at rt. After 5 min, 3-aminopiperidine-2,6-dione hydrochloride (257 mg, 1.56 mmol, 1.0 equiv) and DIPEA (1.09 mL, 6.25 mmol, 4.0 equiv) were added. The mixture was stirred for 16 h and partitioned between EtOAc:H₂O (20 mL each). The organic phase was washed with H₂O (5 mL×2), brine (5 mL×1), and the organic phase was dried over Na₂SO₄, filtered, and concentrated. Purification (SiO₂, 0-4% MeOH:CH₂Cl₂) afforded 5-{4-[(benzyloxy)methyl]piperidin-1-yl}-N-(2,6-dioxopiperidin-3-yl)pyridine-2-carboxamide (625 mg, 92%) as a white solid. LCMS: C₂₄H₂₈N₄O₄ requires: 436, found: m/z=437 [M+H]⁺.

### Step 4: N-(2,6-dioxopiperidin-3-yl)-5-[4-(hydroxymethyl)piperidin-1-yl]pyridine-2-carboxamide (HCB28f)

5-{4-[(benzyloxy)methyl]piperidin-1-yl}-N-(2,6-dioxopiperidin-3-yl)pyridine-2-carboxamide (500 mg, 1.15 mmol, 1.0 equiv), acetic acid (196 μL, 3.44 mmol, 3.0 equiv), Pd(OH)₂ (50 mg), and Pd/C (50 mg) were suspended in EtOH (100 mL) under an atmosphere of H₂ (balloon). The mixture was heated to 40° C. for 18 h before being cooled, filtered, and concentrated. Purification (SiO₂, 0→8% MeOH:CH₂Cl₂) afforded N-(2,6-dioxopiperidin-3-yl)-5-[4-(hydroxymethyl)piperidin-1-yl]pyridine-2-carboxamide (233 mg, 58%) as a white solid. ¹H NMR (500 MHz, DMSO-d₆) δ 10.84 (s, 1H), 8.69 (d, J=8.3 Hz, 1H), 8.30 (d, J=3.0 Hz, 1H), 7.83 (d, J=8.8 Hz, 1H), 7.40 (dd, J=9.0, 3.0 Hz, 1H), 4.74 (dq, J=13.3, 6.4, 5.8 Hz, 1H), 3.95 (d, J=12.7 Hz, 2H), 3.28 (d, J=6.1 Hz, 2H), 2.88-2.73 (m, 3H), 2.53 (s, 2H), 2.23-2.11 (m, 1H), 2.01 (d, J=13.1 Hz, 1H), 1.78-1.71 (m, 2H), 1.61 (s, 1H), 1.21 (h, J=11.0, 10.6 Hz, 2H). LCMS: C₁₇H₂₂N₄O₄ requires: 346, found: m/z=347 [M+H]⁺.

### Step 5: N-(2,6-dioxopiperidin-3-yl)-5-(4-formylpiperidin-1-yl)pyridine-2-carboxamide (HCB28)

A mixture of N-(2,6-dioxopiperidin-3-yl)-5-[4-(hydroxymethyl)piperidin-1-yl]pyridine-2-carboxamide (200 mg, 580 μmol, 1.0 equiv) and Et₃N (321 μL, 2.31 mmol, 4.0 equiv) was dissolved in DMSO (500 μL) and CH₂Cl₂ (500 μL). The reaction mixture was cooled to 0° C. and SO₃·pyridine (184 mg, 1.15 equiv, 2.0 equiv, solution in 300 μL DMSO) was added dropwise. The reaction mixture was warmed to rt and stirred for 30 min before sat. aq. NaHCO₃ (5 mL) was added. After one minute the suspension was diluted with CH₂Cl₂ (10 mL) and the aqueous phase was extracted with CH₂Cl₂ (3×10 mL). The combined organics were washed with H₂O (5 mL×2), brine (5 mL×1), dried over Na₂SO₄, filtered, and concentrated. Purification (SiO₂, 0-10% MeOH:CH₂Cl₂, product elutes at 5%) afforded N-(2,6-dioxopiperidin-3-yl)-5-(4-formylpiperidin-1-yl)pyridine-2-carboxamide as a white foam (190 mg, 95%). LCMS: C₁₇H₂₀N₄O₄ requires: 344, found: m/z=345 [M+H]⁺.

### Example 33. 1-(1-(6-((2,6-dioxopiperidin-3-yl)carbamoyl)pyridin-3-yl)piperidin-4-yl)azetidine-3-carboxylic acid (HCB30)

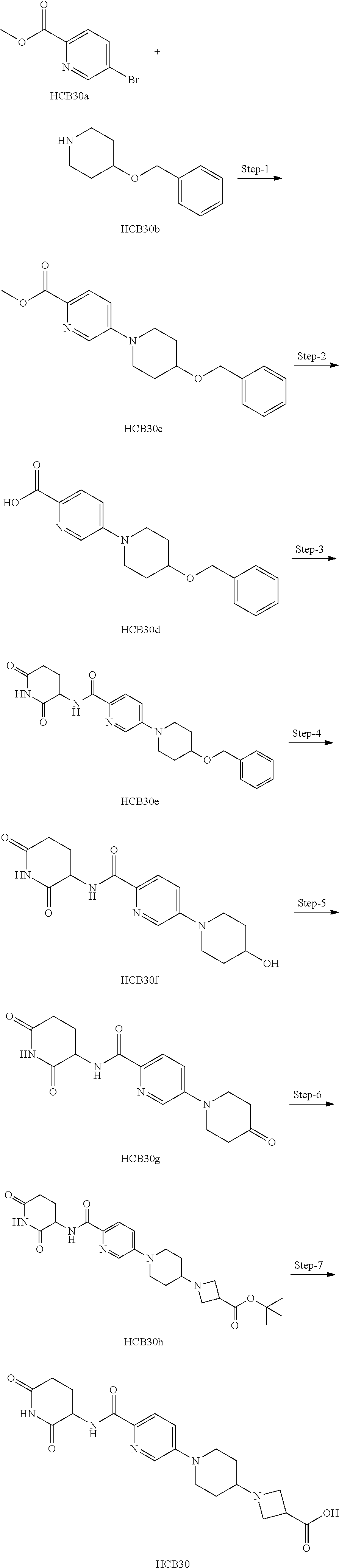

Step 1: Methyl 5-bromopyridine-2-carboxylate (339 mg, 1.6 mmol), 4-(benzyloxy)piperidine (300 mg, 1.6 mmol), Xantphos (91 mg, 0.16 mmol), Pd₂(dba)₃ (144 mg, 0.16 mmol), and Cs₂CO₃ (1.5 g, 4.7 mmol) were deposited in dioxane (3 mL). A vacuum was pulled on the flask until the mixture bubbled, and the headspace was backfilled with Ar for three cycles. The reaction mixture was heated at 100° C. for 30 min and then filtered through Celite. The solid was washed with EtOActhen the filtrate was concentrated in vacuo. Flash chromatography (SiO₂, 0-75% EtOAc:hexanes gradient elution) provided the desired product (205 mg, 0.63 mmol, 40%). LCMS: C₁₉H₂₂N₂O₃ requires: 326, found: m/z=327 [M+H]⁺.

Step 2: A solution of methyl 5-[4-(benzyloxy)piperidin-1-yl]pyridine-2-carboxylate (205 mg, 0.63 mmol) in MeOH (1.5 mL), THE (1.5 mL), and water (0.42 mL) was added to LiOH·H₂O (100 mg, 2.5 mmol). After stirring for 16 h, the volatiles were removed under reduced pressure, the crude material was acidified with 1 N aqueous HCl, and then extracted with EtOAc. The combined organic layer was dried over Na₂SO₄, and concentrated under reduced pressure to afford the desired product (139 mg, 0.45 mmol, 71%). LCMS: C₁₈H₂₀N₂O₃ requires: 312, found: m/z=313 [M+H]⁺.

Step 3: To a solution of 5-[4-(benzyloxy)piperidin-1-yl]-N-(2,6-dioxopiperidin-3-yl)pyridine-2-carboxamide (139 mg, 0.44 mmol), 3-aminopiperidine-2,6-dione hydrochloride (73 mg, 0.44 mmol), and HATU (254 mg, 0.67 mmol) in DMF (1.0 mL) was added i-Pr₂NEt (388 μL, 2.2 mmol). After stirring at room temperature for 2 h, the reaction was quenched with H₂O, and the reaction mixture was extracted with EtOAc. The combined organic layer was washed with saturated aqueous NaCl, dried over Na₂SO₄, and concentrated under reduced pressure. Flash chromatography (SiO₂, 0-5% MeOH:DCM gradient elution) afforded the desired product (207 mg, 0.49 mmol, 50%). LCMS: C₂₃H₂₆N₄O₄ requires: 422, found: m/z=423 [M+H]⁺.

Step 4: A solution of 5-[4-(benzyloxy)piperidin-1-yl]-N-(2,6-dioxopiperidin-3-yl)pyridine-2-carboxamide (206 mg, 0.49 mmol) in EtOH (5 mL) and DCM (5 mL) was stirred under H₂ (1 atm) in the presence of Pd/C (103 mg) and Pd(OH)₂ (103 mg) at rt for three days. The reaction mixture was filtered through Celite and concentrated under reduced pressure to afford the desired product (81 mg, 0.24 mmol, 50%). LCMS: C₁₆H₂₀N₄O₄ requires: 332, found: m/z=333 [M+H]⁺.

Step 5: To a mixture of 2-(2,6-dioxopiperidin-3-yl)-5-(3-hydroxyazetidin-1-yl)isoindole-1,3-dione (39 mg, 0.12 mmol) in DMSO (0.3 mL) and DCM (0.3 mL) was added Et₃N (164 μL, 1.1 mmol) and SO₃ pyridine (83 mg, 0.52 mmol). The reaction mixture was allowed to stir at room temperature for 20 h, and was then concentrated under a positive flow of N₂. Flash chromatography afforded the desired product (22 mg, 0.066 mmol, 30%). LCMS: C₁₆H₁₈N₄O₄ requires: 330, found: m/z=331 [M+H]⁺.

Step 6: A solution of N-(2,6-dioxopiperidin-3-yl)-5-(4-oxopiperidin-1-yl)pyridine-2-carboxamide (23 mg, 0.07 mmol) and tert-butyl azetidine-3-carboxylate (11 mg, 0.07 mmol) in THF (0.20 mL) was allowed to stir at rt for 10 min. NaBH(OAc)₃ (22 mg, 0.1 mmol) was added, and the reaction mixture was allowed to stir at rt for 16 h. The reaction was quenched with 28-30% aqueous NH₄OH, extracted with EtOAc, washed with saturated aqueous NaCl, dried over Na₂SO₄, and concentrated under reduced pressure. Flash chromatography (SiO₂, 0-10% MeOH:DCM gradient elution) provided the desired product (18 mg, 57%). LCMS: C₂₄H₃₃N₅O₅ requires: 472, found: m/z=473 [M+H]⁺.

Step 7: Using the general t-Bu deprotection procedure and treating tert-butyl 1-(1-{6-[(2,6-dioxopiperidin-3-yl)carbamoyl]pyridin-3-yl}piperidin-4-yl)azetidine-3-carboxylate (10 mg, 0.02 mmol) with 2 N HCl in dioxane provided the desired product which was used without further purification. LCMS: C₂₀H₂₅N₅O₅ requires: 414, found: m/z=415 [M+H]⁺.

### Example 34. 1-(6-((2,6-dioxopiperidin-3-yl)carbamoyl)pyridin-3-yl)piperidine-4-carboxylic acid (HCB31)

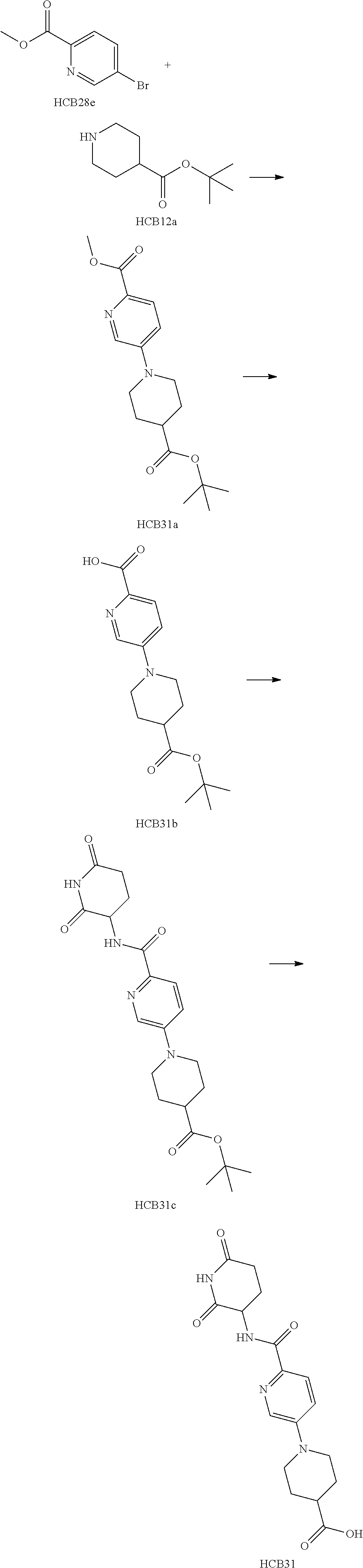

Step 1: Methyl 5-bromopyridine-2-carboxylate (583 mg, 2.7 mmol), tert-butyl piperidine-4-carboxylate (500 mg, 2.7 mmol), Xantphos (156 mg, 0.27 mmol), Pd₂(dba)₃ (247 mg, 0.27 mmol), and Cs₂CO₃ (2.6 g, 8.1 mmol) were deposited in dioxane (5.4 mL). A vacuum was pulled on the flask until the mixture bubbled, and the headspace was backfilled with Ar for three cycles. The reaction mixture was heated at 100° C. for 30 min and then filtered through Celite. The solid was washed with EtOAc. The volatiles were concentrated under reduce pressure. Flash chromatography (SiO₂, 0-75% EtOAc:hexanes gradient elution) provided the desired product (380 mg, 1.2 mmol, 44%). LCMS: C₁₇H₂₄N₂O₄ requires: 320, found: m/z=321 [M+H]⁺.

Step 2: To a solution of methyl 5-(4-(tert-butoxycarbonyl)piperidin-1-yl)picolinic acid (379 mg, 1.18 mmol) in MeOH (3 mL), THE (3 mL), and water (0.8 mL) was added NaOH (189 mg, 4.7 mmol). After stirring for one hour, the volatiles were removed under reduced pressure, and the reaction mixture was acidified with 1 N aqueous HCl, and extracted with EtOAc. The combined organic layer was dried over Na₂SO₄, and concentrated under reduced pressure to afford the desired product (198 mg, 0.65 mmol, 55%). LCMS: C₁₆H₂₂N₂O₄ requires: 306, found: m/z=307 [M+H]⁺.

Step 3: To a solution of 5-(4-(tert-butoxycarbonyl)piperidin-1-yl)picolinic acid (198 mg, 0.65 mmol), 3-aminopiperidine-2,6-dione hydrochloride (106 mg, 0.65 mmol), and HATU (369 mg, 0.97 mmol) in DMF (1.6 mL) was added i-Pr₂NEt (563 μL, 3.2 mmol). After stirring at room temperature for 16 h, the reaction was quenched with H₂O, and extracted with EtOAc. The combined organic layer was washed with saturated aqueous NaCl, dried over Na₂SO₄, and concentrated under reduced pressure. Flash chromatography (SiO₂, 0-5% MeOH:DCM gradient elution) afforded the desired product (539 mg, 1.3 mmol, quant.). LCMS: C₂₁H₂₈N₄O₅ requires: 416, found: m/z=417 [M+H]⁺.

Step 4: Using the general t-Bu deprotection procedure and treating tert-butyl 1-(6-((2,6-dioxopiperidin-3-yl)carbamoyl)pyridin-3-yl)piperidine-4-carboxylate (71 mg, 0.17 mmol) with 2N HCl in dioxane provided the desired product. The crude product was used without purification. LCMS: C₁₇H₂₀N₄O₅ requires: 360, found: m/z=361 [M+H]⁺.

### Example 35. 1-(1-(5-((2,6-dioxopiperidin-3-yl)amino)pyridin-2-yl)piperidin-4-yl)azetidine-3-carboxylic acid (HCB32)

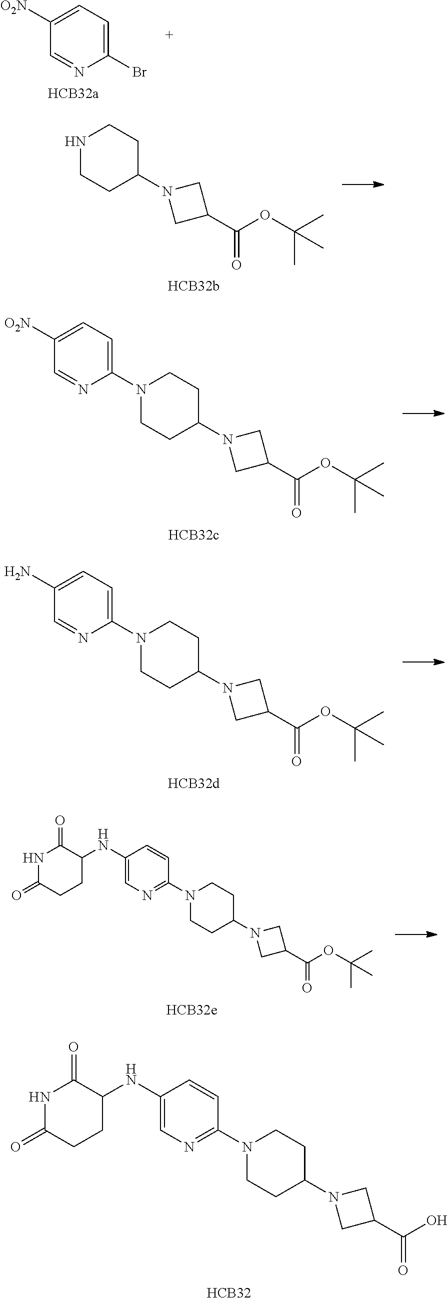

Step 1: To a solution of tert-butyl 1-(piperidin-4-yl) azetidine-3-carboxylate (43 mg, 0.18 mmol) and 2-chloro-5-nitropyridine (45 mg, 0.12 mmol) in THE (360 μL) was added i-Pr₂NEt (93 μL, 0.54 mmol). The reaction mixture was stirred at rt for 16 h, and concentrated under reduced pressure. Silica gel flash chromatography with 0-100% EtOAc:hexanes gradient elution, provided the desired product (45 mg, 0.12 mmol, 69%). LCMS: C₁₈H₂₆N₄O₄ requires: 362, found: m/z=363 [M+H]⁺.

Step 2: A solution of tert-butyl 1-[1-(5-nitropyridin-2-yl)piperidin-4-yl]azetidine-3-carboxylate (45 mg, 0.12 mmol) in EtOH (0.6 mL) and EtOAc (0.6 mL) was stirred under H₂ (one atm) in the presence of Pd/C (12 mg) at rt for one hour. The reaction mixture was filtered through Celite and concentrated under reduced pressure to afford the desired product (46 mg, 0.14 mmol, quant.). LCMS: C₁₈H₂MN₄O₂ requires: 332, found: m/z=333 [M+H]⁺.

Step 3: To a solution of tert-butyl 1-[1-(5-aminopyridin-2-yl)piperidin-4-yl]azetidine-3-carboxylate (46 mg, 0.14 mmol) and 3-bromopiperidine-2,6-dione (35 mg, 0.18 mmol) in DMF (0.35 mL) was added i-Pr₂NEt (96 μL, 0.55 mmol). The reaction mixture was stirred at rt for 16 h, quenched with H₂O, extracted with EtOAc, and concentrated under reduced pressure. Flash chromatography (SiO₂, 0-10% MeOH:DCM gradient elution) provided the desired product (29 mg, 0.06 mmol, 47%). LCMS: C₂₃H₃₃N₅O₄ requires: 444, found: m/z=445 [M+H]⁺.

Step 4: Using the general t-Bu deprotection procedure and treating tert-butyl 1-(1-{5-[(2,6-dioxopiperidin-3-yl)amino]pyridin-2-yl}piperidin-4-yl)azetidine-3-carboxylate (29 mg, 0.06 mmol) with HCl provided the desired product (23.5 mg, 0.06 mmol, 94%). LCMS: C₁₉H₂₅N₅O₄ requires: 387, found: m/z=388 [M+H]⁺.

### Intermediate Synthesis: tert-butyl 1-(piperidin-4-yl)azetidine-3-carboxylate

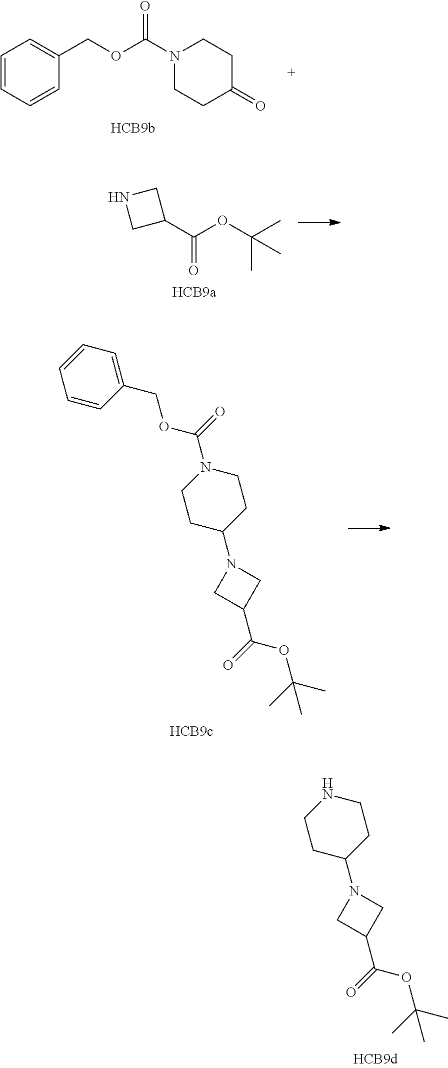 [SMILES: O=C1CCN(C(=O)OCc2ccccc2)CC1 ; CC(C)(C)OC(=O)C1CNC1 ; CC(C)(C)OC(=O)C1CN(C2CCN(C(=O)OCc3ccccc3)CC2)C1 ; CC(C)(C)OC(=O)C1CN(C2CCNCC2)C1]

Step 1: A solution of tert-butyl azetidine-3-carboxylate (1.0 g, 6.4 mmol) and benzyl 4-oxopiperidine-1-carboxylate (1.5 g, 6.4 mmol) in THE (12.7 mL) was stirred at rt for 10 min. NaBH(OAc)₃ (2.0 g, 9.5 mmol) was added, and the reaction mixture was allowed to stir at rt for 16 h. The reaction was quenched with 28-30% aqueous NH₄OH, extracted with EtOAc, washed with saturated aqueous NaCl, dried over Na₂SO₄, and concentrated under reduced pressure. Flash chromatography (SiO₂, 0-100% EtOAc:hexanes gradient elution) provided the desired product (1.8 g, 4.9 mmol, 78%). LCMS: C₂₁H₃₀N₂O₄ requires: 374, found: m/z=375 [M+H]⁺.

Step 2: A solution of benzyl 4-[3-(tert-butoxycarbonyl) azetidin-1-yl]piperidine-1-carboxylate (300 mg, 0.80 mmol) in MeOH (8 mL) was stirred under H₂ (one atm) in the presence of Pd/C (30 mg) at room temperature for one hour. The reaction mixture was filtered through Celite and concentrated under reduced pressure to afford the desired product (177 mg, 0.74 mmol, 92%). LCMS: C₁₃H₂₄N₂O₂ requires: 240, found: m/z=241 [M+H]⁺.

### Example 36. 3-(4-(1-(piperidin-4-ylmethyl)piperidin-4-yl)phenyl)piperidine-2,6-dione (HCB33)

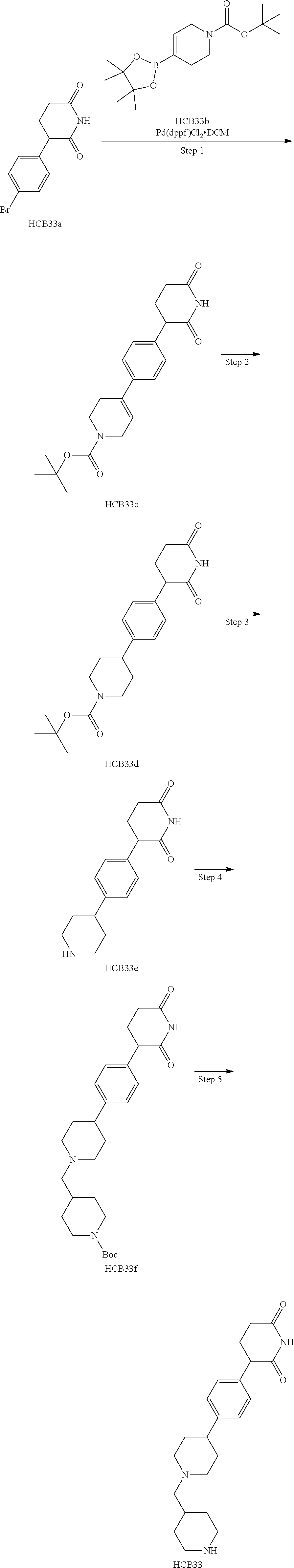

### Step 1: tert-butyl 4-[4-(2,6-dioxopiperidin-3-yl)phenyl]-1,2,3,6-tetrahydropyridine-1-carboxylate (HCB33c)

To an argon purged flask containing N-Boc-1,2,5,6-tetrahydropyridine-4-boronic acid pinacol ester (1.7 g, 5.22 mmol, 1.0 equiv), K₂CO₃ (2.2 g, 15.66 mmol, 3.0 equiv), and Pd(dppf)Cl₂·DCM (0.2 g, 0.26 mmol, 0.05 equiv) in DMF (15 mL, 0.35 M) was added 3-(4-bromophenyl)-piperidine-2,6-dione (1.4 g, 5.22 mmol, 1.0 equiv). The mixture was heated at 85° C. under argon overnight (the reaction was monitored by UPLC). The mixture was filtered from insoluble materials, and all volatiles were evaporated in vacuo. The resulting residue was purified by flash chromatography (20 to 100% EtOAc in Hexane) to give the title product as a white solid (1.35 g, 66%). ESI (−) [M−H]⁻=369.10. ¹H NMR (300 MHz, Chloroform-d), δ 7.98 (s, 1H), 7.40 (d, J=8.4 Hz, 2H), 7.20 (d, J=8.4 Hz, 2H), 6.06 (m, 1H), 4.11 (m, 2H), 3.78 (m, 1H), 3.65 (t, J=5.7 Hz, 2H), 2.75 (m, 2H), 2.53 (s, 2H), 2.34 (s, 2H), 1.51 (s, 9H).

### Step 2: tert-Butyl 4-[4-(2,6-dioxopiperidin-3-yl)phenyl]piperidine-1-carboxylate (HCB33d)

A suspension of benzyl 4-{4-[(tert-butoxy)carbonyl]phenyl}-1,2,3,6-tetrahydropyridine-1-carboxylate (1.411 g, 3.618 mmol, 1.0 equiv) and 5% Pd(OH)₂ (0.07 equiv) in THF (80.38 mL, 0.045 M) was vigorously stirred under H₂ atmosphere (balloon) for 3 h. Reduction progress was monitored by UPLC and NMR. The solids were filtered, washed with THF and the solution was evaporated to give a white crystalline product (1.26 g, 89%). LCMS: 254 nm, Rt=3.73 min, 96.21%, ESI (−) [M−H]⁻=371.59. ¹H NMR (300 MHz, Chloroform-d), δ 8.04 (s, 1H), 7.22 (d, J=8.2 Hz, 2H), 7.16 (d, J=8.2 Hz, 2H), 4.26 (d, J=13.3 Hz, 2H), 3.78 (m, 1H), 2.88-2.61 (m, 5H), 2.31 (m, 2H), 1.65 (m, 2H), 1.65 (m, 2H), 1.50 (s, 9H).

Step 3: tert-butyl 4-{4-[(3R)-2,6-dioxopiperidin-3-yl]phenyl}piperidine-1-carboxylate (40.00 mg, 0.11 mmol) was dissolved in 4 N HCl in dioxane (3 mL) and stirred for 30 minutes. The volatiles were removed, and the desired product was placed under vacuum to dryness. The crude material was used in the next step without purification. LCMS: C₁₆H₂₀N₂O₂ requires: 272, found: m/z=273 [M+H]⁺.

Step 4: To a solution of (3R)-3-[4-(piperidin-4-yl)phenyl]piperidine-2,6-dione (38.00 mg, 0.14 mmol) and t-butyl 4-formylpiperidine-1-carboxylate (30 mg, 0.14 mmol) in DMSO (1 mL), TEA (20 mL, 0.14 mmol) and sodium triacetoxyborohydride (89 mg, 0.42 mmol) were added and the reaction was stirred for 30 mins. LCMS indicated the completion of reaction. The reaction was quenched with water and extracted with EtOAc. The organic layer was washed with saturated aqueous NaCl, dried over Na₂SO₄, and concentrated under reduced pressure. Silica gel chromatography purification provided the desired product (35 mg, 0.07 mmol, 53%). LCMS: C₂₇H₃₉N₃O₄ requires: 469, found: m/z=470 [M+H]⁺.

Step 5: tert-butyl 4-[(4-{4-[(3R)-2,6-dioxopiperidin-3-yl]phenyl}piperidin-1-yl)methyl]piperidine-1-carboxylate as a colorless oil (35 mg, 0.16 mmol) was dissolved in DCM (1 mL) and 4N HCl in dioxane (2 mL) was added, The reaction was stirred for one hour. All the solvents were removed under reduced pressure to afford 3-{4-[1-(piperidin-4-ylmethyl)piperidin-4-yl]phenyl}piperidine-2,6-dione hydrogen chloride salt (quantitative yield). LCMS: C₂₄H₃₁N₃O₂ requires: 369, found: m/z=370 [M+H]⁺.

### Example 37. 3-4-bromophenyl)piperidine-2,6-dione synthesis HCB33a)

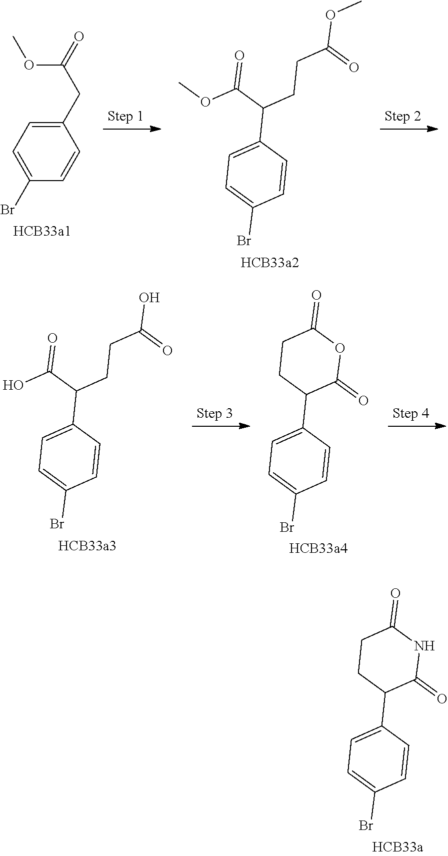

### Step 1: 1,5-dimethyl 2-(4-bromophenyl)pentanedioate (HCB33a2)

A solution of methyl 4-bromophenylacetate (10.2 g, 43.65 mmol, 1.0 equiv) in anhydrous toluene (30 mL) was added dropwise to a suspension of potassium tert-butoxide (1.01 g, 8.731 mmol, 0.2 equiv) in toluene (50.0 mL) under nitrogen atmosphere at −70° C. The resulting mixture was stirred for fifteen minutes and a solution of 18-crown-6-ether (1.1 g, 4.36 mmol, 0.1 equiv) in toluene (10 mL) was added. Then, a solution of methyl acrylate (3.8 g, 43.65 mmol, 1 equiv) in toluene (20 mL) was added and the slurry reaction mixture was stirred for one hour at low temperature and two hours at ambient temperature. Afterward, the reaction was poured into a saturated aqueous HCl solution (100 mL), and the product was extracted with diethyl ether, dried over Na₂SO₄, concentrated in vacuo, and purified by flash chromoatography (0 to 15% EtOAc in Hexane) to give the desired compound as a colorless oil (9.2 g, 64%). ¹H NMR (300 MHz, Chloroform-d), δ 7.45 (d, J=8.4 Hz, 2H), 7.16 (d, J=8.4 Hz, 2H), 3.74-3.50 (m, 7H), 2.37 (m, 1H), 2.26 (m, 2H), 2.10 (m, 1H).

### Step 2: 2-(4-bromophenyl)pentanedioic acid (HCB33a3)

A solution of 1,5-dimethyl-2-(4-bromophenyl)pentanedioate (4.7 g, 14.27 mmol, 1 equiv) and potassium hydroxide (2.1 g, 35.69 mmol, 2.5 equiv) in THF:Water (1:1) (30 mL, 0.4 M) was stirred at room temperature for 2 h, and then one hour at 90° C. The reaction progress was monitored by NMR or UPLC. Upon completion, the resulting solution was neutralized with dilute HCl to pH=5, extracted with diethyl ether, and dried over Na₂SO₄. All volatiles were evaporated at reduced pressure to give an off-white solid product (3.95 g, 92%). ESI (−) [M−H]⁻=286.75. ¹H NMR (300 MHz, Chloroform-d), δ 7.49 (d, J=8.4 Hz, 2H), 7.21 (d, J=8.4 Hz, 2H), 3.65 (m, 1H), 2.50-2.36 (m, 3H), 2.10 (m, 1H).

### Step 3: 3-(4-bromophenyl)oxane-2,6-dione (HCB33a4)

A solution of 2-(4-bromophenyl)-pentanedioic acid (0.526 g, 1.74 mmol, 1 equiv) in acetic anhydride (3.28 ml, 34.82 mmol, 20 equiv) was heated at reflux for 1.5 h. All volatiles were removed by distillation at low pressure and the crude residue was co-evaporated twice with toluene to give the desired compound (0.43 g, 83%). ¹H NMR (300 MHz, Chloroform-d), δ 7.55 (d, J=8.4 Hz, 2H), 7.14 (d, J=8.4 Hz, 2H), 3.88 (m, 1H), 3.09-2.77 (m, 2H), 2.30 (m, 2H).

### Step 4: 3-(4-bromophenyl)piperidine-2,6-dione (HCB33a)

A mixture of the 3-(4-bromophenyl)oxane-2,6-dione (3.0 g, 10.0 mmol, 1.0 equiv) and well-ground ammonium carbonate (4.9 g, 50.17 mmol, 5.0 equiv) was heated to 190-200° C. for 120 minutes in a 100 mL flask equipped with an air condenser. The reaction mixture became a clear liquid that solidified upon cooling. The residue was triturated with warm 50% aqueous ethanol which converted to a slurry of crystals. The crystals were collected, washed with 50% aqueous ethanol, and dried to give a gray crystalline solid (2.0 g, 71%). LCMS: 254 nm, Rt=2.42 min, 98.68%, ESI (−) [M−H]⁻=267.70. ¹H NMR (300 MHz, DMSO-d₆), δ 10.86 (s, 1H), 7.53 (d, J=8.4 Hz, 2H), 7.20 (d, J=8.4 Hz, 2H), 3.88 (m, 1H), 2.62 (m, 1H), 2.54 (s, 1H), 2.17 (m, 1H), 2.01 (m, 1H).

### Example 38. 3-(4-(4-(piperazin-1-yl)but-1-yn-1-yl)phenyl)piperidine-2,6-dione (HCB34)

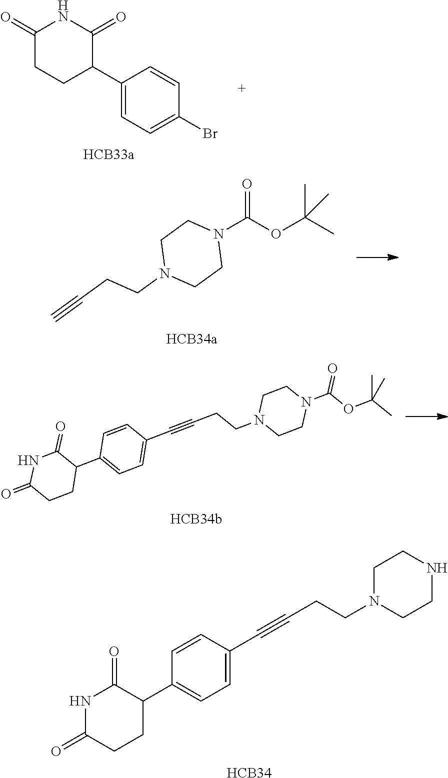

Step-1: To a solution of 3-(4-bromophenyl)piperidine-2,6-dione (50.00 mg, 0.19 mmol) and tert-butyl 4-(but-3-yn-1-yl)piperazine-1-carboxylate (89 mg, 0.37 mmol) in THE (2 mL) was added palladium bis(triphenylphosphine) chloride dichloromethane (13.09 mg, 0.02 mmol), copper(I) iodide (7.10 mg, 0.04 mmol), and triethylamine (0.36 mL, 0.26 g, 2.61 mmol). Then, nitrogen gas was bubbled through the mixture for five minutes. The reaction mixture was heated at 60° C. for one hour. Additional equivalent of tert-butyl 4-(but-3-yn-1-yl)piperazine-1-carboxylate (45 mg, 0.19 mmol) was added, and the reaction was heated at 60° C. for 16 hours. The reaction mixture was diluted with DCM (25 mL) and washed sequentially with water (3 mL), 10% citric acid in water (3 mL), and saturated brine (3 mL). The organic layer was dried with Na₂SO₄, filtered, and evaporated under reduced pressure to afford crude product. The crude product was purified by silica gel column chromatography (ISCO elution gradient 0 to 5% MeOH in DCM) to afford tert-butyl 4-{4-[4-(2,6-dioxopiperidin-3-yl)phenyl]but-3-yn-1-yl}piperazine-1-carboxylate (36 mg, 45%). LCMS: C₂₄H₃₁N₃O₄ requires: 425.2, found: m/z=426.5 [M+H]⁺.

Step-2: tert-butyl 4-{4-[4-(2,6-dioxopiperidin-3-yl)phenyl]but-3-yn-1-yl}piperazine-1-carboxylate (36 mg, 0.08 mmol) was dissolved in 5% trifluoroacetic acid in hexafluoro isopropanol solution (5 mL), and left to stand for 30 minutes. Trifluoroacetic acid and hexafluoroisopropanol were removed. The residue was placed under vacuum to dryness to provide 3-{4-[4-(piperazin-1-yl)but-1-yn-1-yl]phenyl}piperidine-2,6-dione (quantitative yield). LCMS: C₁₉H₂₃N₃O₂ requires: 325.2, found: m/z=326.5 [M+H]⁺.

### Example 39. 3-(4-(4-(piperidin-4-ylmethyl)piperazin-1-yl)phenyl)piperidine-2,6-dione synthesis (HCB35)

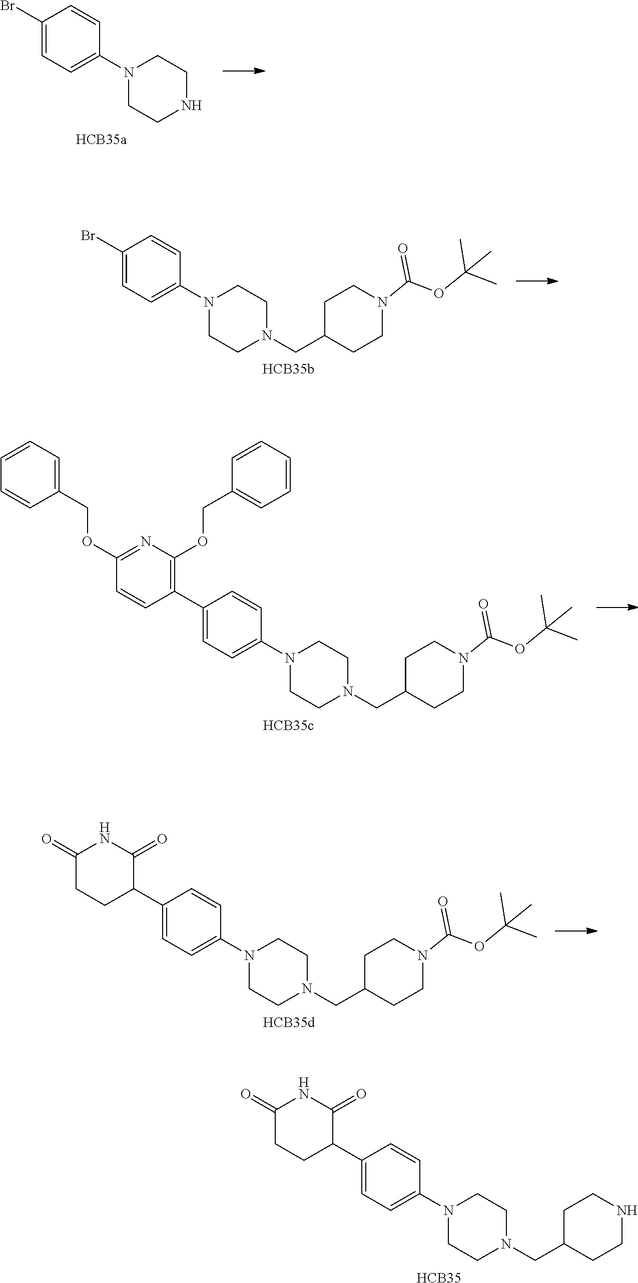

Step 1: To a solution of 1-(4-bromophenyl)piperazine (75.00 mg, 0.31 mmol) and tert-butyl 4-formylpiperidine-1-carboxylate (66.34 mg, 0.31 mmol) in DCM (2 mL) was added N,N-diisopropylethylamine (0.16 mL, 120.60 mg, 0.93 mmol), and sodium triacetoxyborohydride (396 mg, 1.86 mmol) as powder. The reaction was stirred for 30 mins when LCMS indicated completion of reaction. The crude product was dissolved in DCM (15 mL), washed with water, dried, and concentrated. Silica gel column purification (MeOH:DCM (2% TEA) 0-8% elution) provided the desired product (111 mg, 0.25 mmol, 81%). LCMS: C₂₁H₃₂BrN₃O₂ requires: 438, found: m/z=439 [M+H]⁺.

Step 2: To a solution of tert-butyl 4-{[4-(4-bromophenyl)piperazin-1-yl]methyl}piperidine-1-carboxylate (111 mg, 0.25 mmol) and tert-butyl 4-{[4-(4-bromophenyl)piperazin-1-yl]methyl}piperidine-1-carboxylate (85 mg, 0.25 mmol) in dioxane (2 mL) and water (0.5 mL) was added Cs₂CO₃ (247 mg, 0.76 mmol) and Pd(dppf)Cl₂·DCM (41 mg, 0.05 mmol). Nitrogen gas was bubbled through the reaction mixture and then reaction was heated at 100° C. for one hour when LCMS indicated reaction completion. The reaction mixture was dissolved in EtOAc, washed with brine, dried over Na₂SO₄, and concentrated. ISCO silica gel column purification eluting with MeOH:DCM (0-10%) provided the desired product (68 mg, 0.1 mmol, 41%). LCMS: C₄₀H₄₈N₄O₄ requires: 648.4, found: m/z=649.7 [M+H]⁺.

Step 3: tert-butyl 4-[(4-{4-[2,6-bis(benzyloxy)pyridin-3-yl]phenyl}piperazin-1-yl)methyl]piperidine-1-carboxylate (45 mg, 0.07 mmol) was added to a 25 mL round bottom flask. Then wet Pd on carbon (15 mg) and EtOH (3 mL) was added to dissolve starting material. The solution was stirred under a hydrogen balloon overnight. The reaction mixture was filtered through celite and the filtrate concentrated under reduced pressure. The crude product was purified by ISCO silica gel column chromatography eluting with MeOH:DCM having 2% TEA (0-12%) which provided the desired product. (18 mg, 0.04 mmol, 55%). LCMS: C₂₆H₂₈N₄O₄ requires: 470, found: m/z=471 [M+H]⁺.

Step 4: tert-butyl 4-({4-[4-(2,6-dioxopiperidin-3-yl)phenyl]piperazin-1-yl}methyl)piperidine-1-carboxylate (18 mg, 0.04 mmol) was dissolved in 4 N HCl in dioxane (2 mL). After 1 hr, the volatiles were removed and the residue was placed under vacuum to dryness to afford 3-{4-[4-(piperidin-4-ylmethyl)piperazin-1-yl]phenyl}piperidine-2,6-dione (quantitative yield). LCMS: C₂₁H₃₀N₄O₂ requires: 370, found: m/z=371 [M+H]⁺.

### Example 40. 3-(4-(4-(piperazin-1-ylmethyl)piperidin-1-yl)phenyl)piperidine-2,6-dione (HCB36)

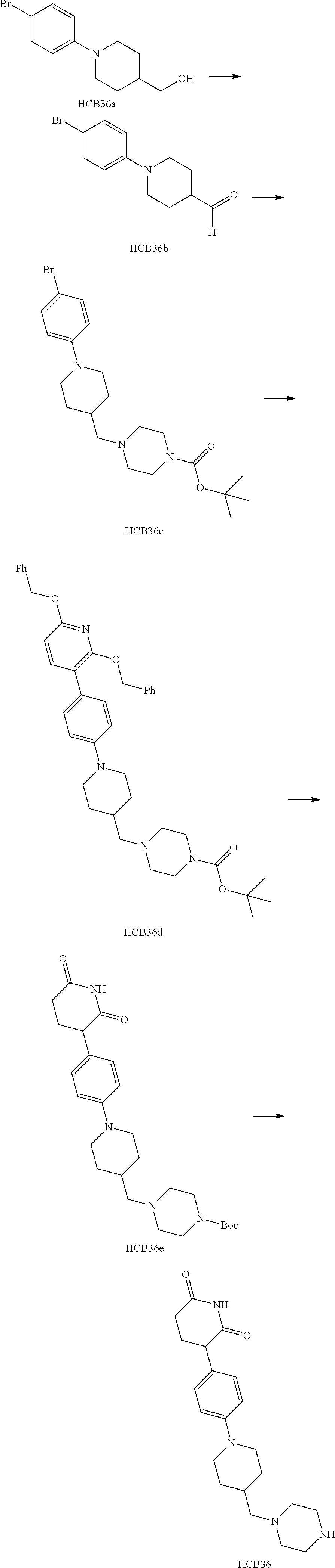

Step 1: To a solution of [1-(4-bromophenyl)piperidin-4-yl]methanol (240.00 mg, 0.89 mmol) in DMSO (1.5 mL) was added triethylamine (2.50 mL, 1.80 g, 17.77 mmol) and then sulfur trioxide pyridine complex (1.41 g, 8.88 mmol). The reaction was stirred at rt for 30 mins when TLC showed no starting material left. The product was dissolved in EtOAc (50 mL) and the organic layer was washed water (2 mL×2). The organic layer was dried and concentrated. The crude material was used in the next step without purification.

Step 2: To a solution of 1-(4-bromophenyl)piperidine-4-carbaldehyde (230.00 mg, 0.86 mmol) and tert-butyl piperazine-1-carboxylate (0.16 g, 0.86 mmol) in DCM (10 mL) was added sodium triacetoxyborohydride (0.55 g, 0.257 mmol), and the reaction was stirred for 0.5 hr. The reaction mixture was quenched with 5% sodium bicarbonate solution and extracted with CH₂Cl₂. The organic layer was dried and concentrated under reduced pressure. Silica gel column purification eluting with 0-80% EtOAc in hexane provided the desired product (221 mg, 0.5 mmol, 59%). LCMS: C₂₁H₃₂BrN₃O₂ requires: 437.2, found: m/z=439 [M+H]⁺.

Step 3: tert-butyl 4-[(1-{4-[2,6-bis(benzyloxy)pyridin-3-yl]phenyl}piperidin-4-yl)methyl]piperazine-1-carboxylate. The compound was prepared following the same reaction procedure as in Step 2 of Example 38. LCMS: C₄₀H₄₈N₄O₄ requires: 648.4, found: m/z=649.7 [M+H]⁺.

Step 4: tert-butyl 4-((1-(4-(2,6-dioxopiperidin-3-yl)phenyl)piperidin-4-yl)methyl)piperazine-1-carboxylate. The compound was prepared following the same reaction procedure as in Step 2 of Example 38. LCMS: C₂₆H₃₈N₄O₄ requires: 470.3, found: m/z=471.6 [M+H]⁺.

Step 5: 3-(4-(4-(piperazin-1-ylmethyl)piperidin-1-yl)phenyl)piperidine-2,6-dione. The compound was prepared following the same reaction procedure as in Step 2 of Example 38. LCMS: C₂₁H₃₀N₄O₂ requires: 370.2, found: m/z=371.3 [M+H]⁺.

### Example 41. 3-(5-(4-(piperidin-4-ylmethyl)piperazin-1-yl)pyridin-2-yl)piperidine-2,6-dione (HCB37)

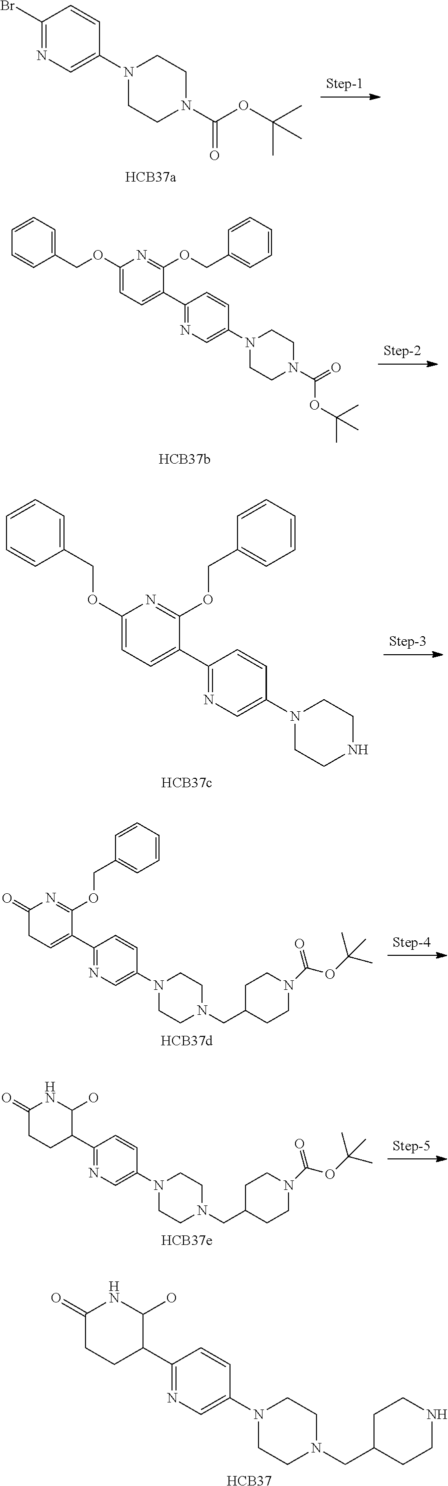

Step 1: A solution of tert-butyl 4-(6-bromopyridin-3-yl)piperazine-1-carboxylate (250.00 mg, 0.73 mmol), 2,6-bis(benzyloxy)pyridin-3-ylboronic acid (244.84 mg, 0.73 mmol), cesium carbonate (714.02 mg, 2.19 mmol), and Pd(dppf)Cl₂·DCM in dioxane (4 mL) and water (1 mL) was heated at 100° C. for two hours. The reaction mixture was cooled, dissolved in EtOAc (30 mL), and washed with brine. The organic layer was dried over sodium sulfate and concentrated. ISCO silica gel column purification eluting with EtOAc in hexane (5-100%) provided tert-butyl 4-[2′,6′-bis(benzyloxy)-[2,3′-bipyridin]-5-yl]piperazine-1-carboxylate (168 mg, 41%). LCMS: C₃₃H₃₆N₄O₄ requires: 552.3, found: m/z=553.7 [M+H]⁺.

Step 2: tert-butyl 4-[2′,6′-bis(benzyloxy)-[2,3′-bipyridin]-5-yl]piperazine-1-carboxylate (80.00 mg, 0.14 mmol) was dissolved in 4 N HCl in dioxane (4 mL) and the reaction was left to stand for one hour. The volatiles were removed and the crude material was placed under vacuum until dryness to provide 2′,6′-bis(benzyloxy)-5-(piperazin-1-yl)-2,3′-bipyridine HCl salt (68 mg, quantitative yield).

Step 3: To a solution of 2′,6′-bis(benzyloxy)-5-(piperazin-1-yl)-2,3′-bipyridine (60.00 mg, 0.13 mmol) and tert-butyl 4-formylpiperidine-1-carboxylate (28.28 mg, 0.13 mmol) in DCM (1 mL) and DMSO (0.3 mL) as added N,N-diisopropylethylamine (23.16 μL, 0.02 g, 0.13 mmol) and sodium triacetoxyborohydride (84.30 mg, 0.40 mmol). The reaction was stirred for 30 minutes when LCMS showed no starting material remained. The crude was dissolved in DCM (30 mL), washed with water, dried over Na₂SO₄, and concentrated. The crude product was purified by silica gel column chromatography eluting with MeOH in DCM having 2% TEA (0-8%) which provided tert-butyl 4-({4-[2′-(benzyloxy)-6′-oxo-5′H-[2,3′-bipyridin]-5-yl]piperazin-1-yl}methyl)piperidine-1-carboxylate (32 mg, 43%). LCMS: C₃₉H₄₇N₅O₄ requires: 559.3, found: m/z=560.7 [M+H]⁺.

Step 4: A suspension of tert-butyl 4-({4-[2′-(benzyloxy)-6′-oxo-5′H-[2,3′-bipyridin]-5-yl]piperazin-1-yl}methyl)piperidine-1-carboxylate (32 mg, 0.06 mmol) and 10% Pd/C (10 mg) in ethanol (5 mL) was vigorously stirred under a H₂ atmosphere (balloon) for sixteen hours. The solids were filtered, washed with DCM, and the volatiles were removed. The crude product was purified by silica gel column chromatography eluting with MeOH in DCM with 2% TEA (0-10%) which provided tert-butyl 4-((4-(6-(2,6-dioxopiperidin-3-yl)pyridin-3-yl)piperazin-1-yl)methyl)piperidine-1-carboxylate (28 mg, 92%). LCMS: C₂₅H₃₇N₅O₄ requires: 471.6, found: m/z=472.5 [M+H]⁺.

Step 5: tert-butyl 4-((4-(6-(2,6-dioxopiperidin-3-yl)pyridin-3-yl)piperazin-1-yl)methyl)piperidine-1-carboxylate (28 mg, 0.06 mmol) was dissolved in 4 N HCl in dioxane (4 mL) and the reaction was left to stand for one hour. The volatiles were removed and the material was placed under vacuum until dryness to provide 3-(5-(4-(piperidin-4-ylmethyl)piperazin-1-yl)pyridin-2-yl)piperidine-2,6-dione (quantitative yield). LCMS: C₂₀H₂₉N₅O₂ requires: 371.2, found: m/z=372.5 [M+H]⁺.

### Example 42. 3-(5-(4-(piperazin-1-ylmethyl)piperidin-1-yl)pyridin-2-yl)piperidine-2,6-dione synthesis (HCB38)

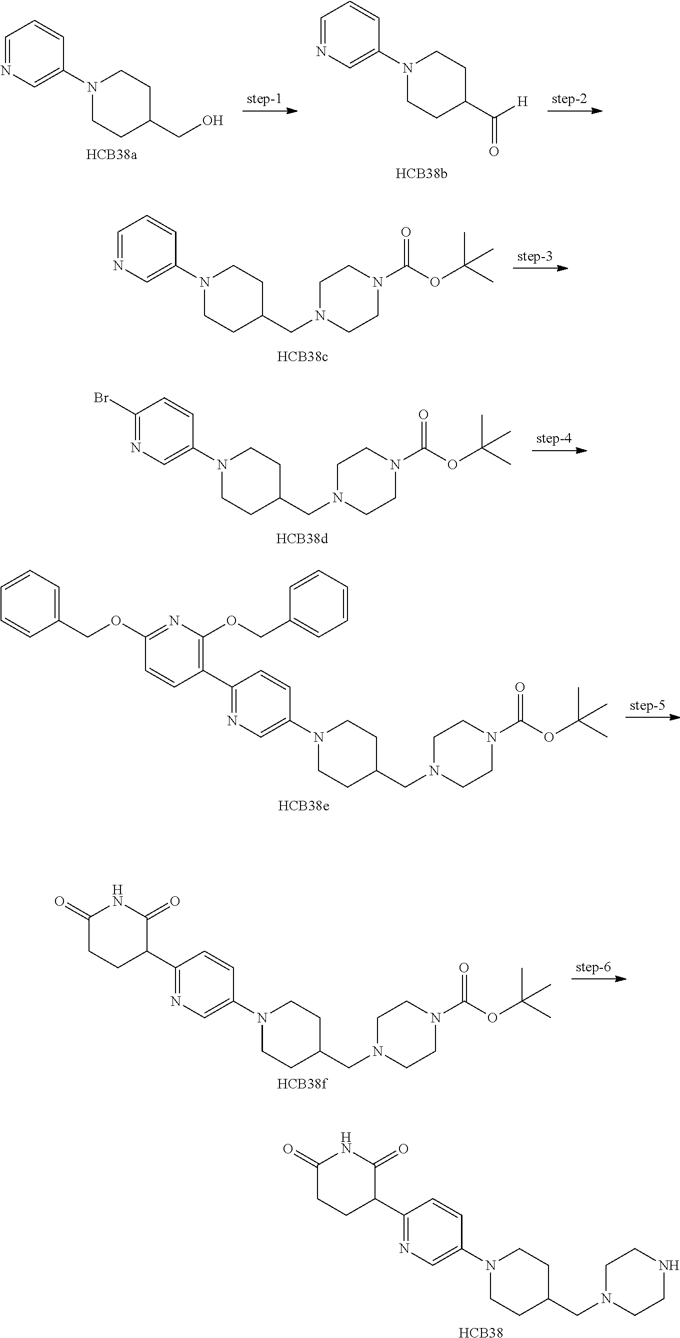

Step 1: To a solution of [1-(pyridin-3-yl)piperidin-4-yl]methanol (300.00 mg, 1.56 mmol) in DMSO (2 mL) was added triethylamine (4.39 mL, 3.16 g, 31.21 mmol), and then sulfur trioxide pyridine complex (2.48 g, 15.60 mmol, powder) was added slowly. The reaction was stirred at rt for 30 minutes when TLC showed no starting material remained. The product was dissolved in EtOAc (50 mL) and the organic solution was washed with water (3 mL×2). The organic layer was dried over Na₂SO₄, filtered, and concentrated to provide 1-(pyridin-3-yl)piperidine-4-carbaldehyde (0.2800 g, 94.3%) as crude product. The crude product was used in the next step without purification.

Step 2: To a solution of 1-(pyridin-3-yl)piperidine-4-carbaldehyde (280.00 mg, 1.47 mmol) and tert-butyl piperazine-1-carboxylate (0.27 g, 1.47 mmol) in DCM (10 mL) was added sodium triacetoxyborohydride (0.94 g, 4.42 mmol) and the reaction was stirred for 30 minutes. The reaction was quenched with sodium bicarbonate solution (2 mL), and the mixture was extracted DCM (50 mL). The organic layer was dried over Na₂SO₄, filtered, and concentrated. Silica gel column purification eluting with 0-10% MeOH:DCM provided the desired product (403 mg, 1.12 mmol, 76%). LCMS: C₂₀H₃₂N₄O₂ requires: 360.3, found: m/z=361.6 [M+H]⁺. ¹H NMR (500 MHz, CDCl₃) δ 8.34 (d, J=2.9 Hz, 1H), 8.09 (dd, J=4.6, 1.4 Hz, 1H), 7.23-7.20 (m, 1H), 7.16 (dd, J=8.5, 4.5 Hz, 1H), 3.72 (d, J=12.4 Hz, 2H), 3.45 (t, J=5.0 Hz, 4H), 2.77 (td, J=12.1, 2.6 Hz, 2H), 2.64 (s, 3H), 2.38 (d, J=5.5 Hz, 5H), 2.25 (d, J=7.1 Hz, 2H), 1.91 (d, J=13.2 Hz, 2H), 1.70 (ddd, J=11.2, 7.5, 3.8 Hz, 1H), 1.49 (s, 9H), 1.36 (qd, J=12.2, 4.1 Hz, 2H).

Step 3: To a solution of tert-butyl 4-{[1-(pyridin-3-yl)piperidin-4-yl]methyl}piperazine-1-carboxylate (403.00 mg, 1.12 mmol) in MeCN (10 mL) was slowly added NBS (238 mg, 1.34 mmol) in MeCN (1 mL) and the reaction was stirred at rt for one hour. The reaction was monitored by LCMS which showed no starting material remained. The volatiles were removed and the crude product was purified by silica gel column chromatography eluting with EtOAc:DCM (0-100%) to afford desired product (242 mg, 0.55 mmol, 49%). LCMS: C₂₀H₃₁BrN₄O₂ requires: 439.2, found: m/z=441.3 [M+H]⁺. ¹H NMR (500 MHz, CDCl₃) δ 8.03 (d, J=3.2 Hz, 1H), 7.30 (d, J=8.1 Hz, 1H), 7.10 (dd, J=8.8, 3.2 Hz, 1H), 3.67 (dt, J=13.0, 3.4 Hz, 2H), 3.45 (t, J=5.1 Hz, 4H), 2.85-2.69 (m, 2H), 2.37 (d, J=5.2 Hz, 4H), 2.24 (d, J=7.2 Hz, 2H), 1.90 (d, J=13.2 Hz, 2H), 1.69 (ddt, J=11.2, 7.6, 3.7 Hz, 1H), 1.49 (s, 9H), 1.42-1.27 (m, 2H).

Step 4: A solution of tert-butyl 4-{[1-(6-bromopyridin-3-yl)piperidin-4-yl]methyl}piperazine-1-carboxylate (242.00 mg, 0.55 mmol) and 2,6-bis(benzyloxy)pyridin-3-ylboronic acid (184 mg, 0.55 mmol) in dioxane (5 mL) and water (1.25 mL) was added cesium carbonate (714.02 mg, 2.19 mmol) and Pd(dppf)Cl₂·DCM. Nitrogen was bubbled through the reaction for five minutes and then the reaction was heated at 100° C. for two hours. The reaction was cooled, diluted with EtOAc (40 mL), and washed with brine. The organic layer was dried over Na₂SO₄, filtered, and concentrated. ISCO silica gel column purification eluting with EtOAc in DCM (20-100%) provided tert-butyl 4-({1-[2′,6′-bis(benzyloxy)-[2,3′-bipyridin]-5-yl]piperidin-4-yl}methyl)piperazine-1-carboxylate (238 mg, 0.37 mmol, 67%). LCMS: C₃₉H₄₇N₅O₄ requires: 649.4, found: m/z=650.8 [M+H]⁺.

Step 5: A suspension of tert-butyl 4-({1-[2′,6′-bis(benzyloxy)-[2,3′-bipyridin]-5-yl]piperidin-4-yl}methyl)piperazine-1-carboxylate (238 mg, 0.37 mmol) and Pd on carbon (90 mg) in ethanol (8 mL) and THE (4 mL) was stirred under hydrogen gas overnight. The solids were filtered and the volatiles were removed. Silica gel column purification eluting with MeOH:DCM (0-8%), provided the desired product (46 mg, 0.1 mmol, 27%). LCMS: C₂₅H₃₇N₅O₄ requires: 471.3, found: m/z=472.5 [M+H]⁺.

Step 6: tert-butyl 4-[(1-{6-[(3R)-2,6-dioxopiperidin-3-yl]pyridin-3-yl}piperidin-4-yl)methyl]piperazine-1-carboxylate was dissolved in DCM (1 mL). 4 N HCl in dioxane (1 mL) was added and the reaction was left to stand for one hour. After the volatiles were removed the desired product was obtained (quantitative yield). LCMS: C₂₀H₂₉N₅O₂ requires: 371.2, found: m/z=372.4 [M+H]⁺.

### Example 43. 3-(6-(4-(piperidin-4-ylmethyl)piperazin-1-yl)pyridin-2-yl)piperidine-2,6-dione (HCB39)

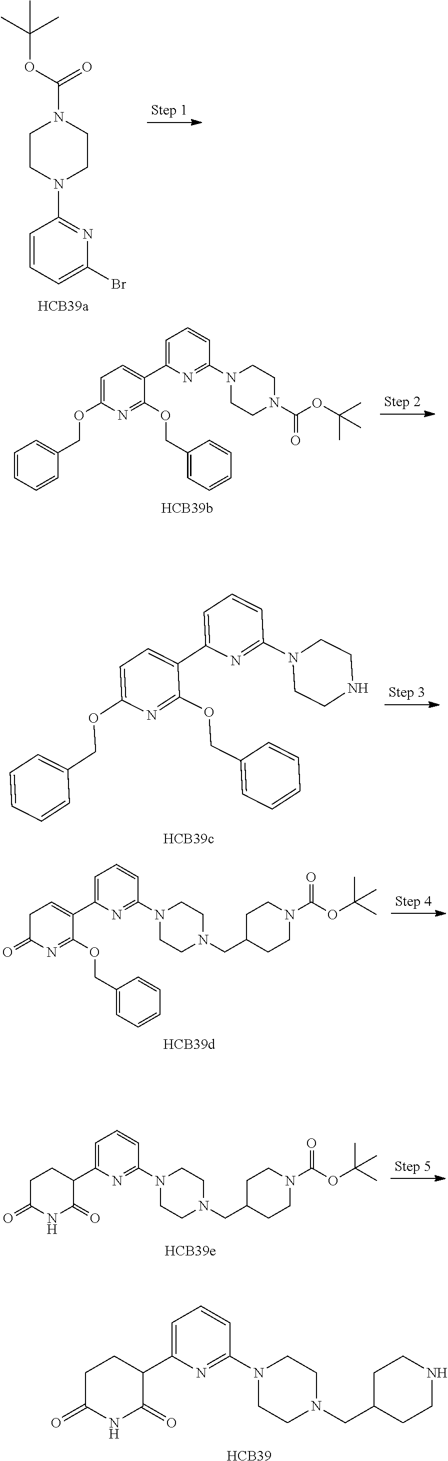

Step 1: The HBC38 Step 4 procedure was adapted to provide tert-butyl 4-(2′,6′-bis(benzyloxy)-[2,3′-bipyridin]-6-yl)piperazine-1-carboxylate. LCMS: C₃₃H₃₆N₄O₄ requires: 552.3, found: m/z=553.5 [M+H]⁺.

Step 2: The HBC38 Step 6 procedure was adapted to provide 2′,6′-bis(benzyloxy)-6-(piperazin-1-yl)-2,3′-bipyridine. LCMS: C₂₈H₂₈N₄O₂ requires: 452.2, found: m/z=453.5 [M+H]⁺.

Step 3: 2′,6′-bis(benzyloxy)-6-(piperazin-1-yl)-2,3′-bipyridine (96.00 mg, 0.21 mmol) and tert-butyl 4-formylpiperidine-1-carboxylate (45.24 mg, 0.21 mmol) were dissolved in 4 ml DCM, sodium triacetoxyborohydride (179.8 mg, 0.85 mmol) was added, stirred for 30 mins. The crude was dissolved in DCM (30 mL), washed with water, dried over Na₂SO₄, and concentrated. The crude product was purified by silica gel column chromatography eluting with 0-80% EtOAc/Hexane, obtained tert-butyl 4-((4-(2′-(benzyloxy)-6′-oxo-5′,6′-dihydro-[2,3′-bipyridin]-6-yl)piperazin-1-yl)methyl)piperidine-1-carboxylate (76 mg, 0.14 mmol, 64%). LCMS: C₃₂H₄₁N₅O₄ requires: 559.3, found: m/z=560.8 [M+H]⁺.

Step 4: A suspension of tert-butyl 4-((4-(2′-(benzyloxy)-6′-oxo-5′,6′-dihydro-[2,3′-bipyridin]-6-yl)piperazin-1-yl)methyl)piperidine-1-carboxylate (76 mg, 0.14 mmol,) and Pd on carbon (22 mg) in ethanol (3 mL) and THF (3 mL) was stirred under hydrogen gas overnight. The solids were filtered and the volatiles were removed. Silica gel column purification eluting with EtOAc, provided the desired product (34 mg, 0.07 mmol, 53%). tert-butyl 4-((4-(2′-(benzyloxy)-6′-oxo-5′,6′-dihydro-[2,3′-bipyridin]-6-yl)piperazin-1-yl)methyl)piperidine-1-carboxylate. LCMS: C₂₅H₃₇N₅O₄ requires: 471.3, found: m/z=472.6 [M+H]⁺.

Step 5: tert-butyl 4-((4-(2′-(benzyloxy)-6′-oxo-5′,6′-dihydro-[2,3′-bipyridin]-6-yl)piperazin-1-yl)methyl)piperidine-1-carboxylate (34 mg, 0.07 mmol) in DCM (1 mL). 4 N HCl in dioxane (2 mL) was added and the reaction was left to stand for one hour. After the volatiles were removed the desired product was obtained (quantitative yield). 3-(6-(4-(piperidin-4-ylmethyl)piperazin-1-yl)pyridin-2-yl)piperidine-2,6-dione. LCMS: C₂₀H₂₉N₅O₂ requires: 371.2, found: m/z=372.5 [M+H]⁺.

### Example 44. 3-(5-(4-(piperidin-4-ylmethyl)piperazin-1-yl)pyridin-3-yl)piperidine-2,6-dione (HCB340)

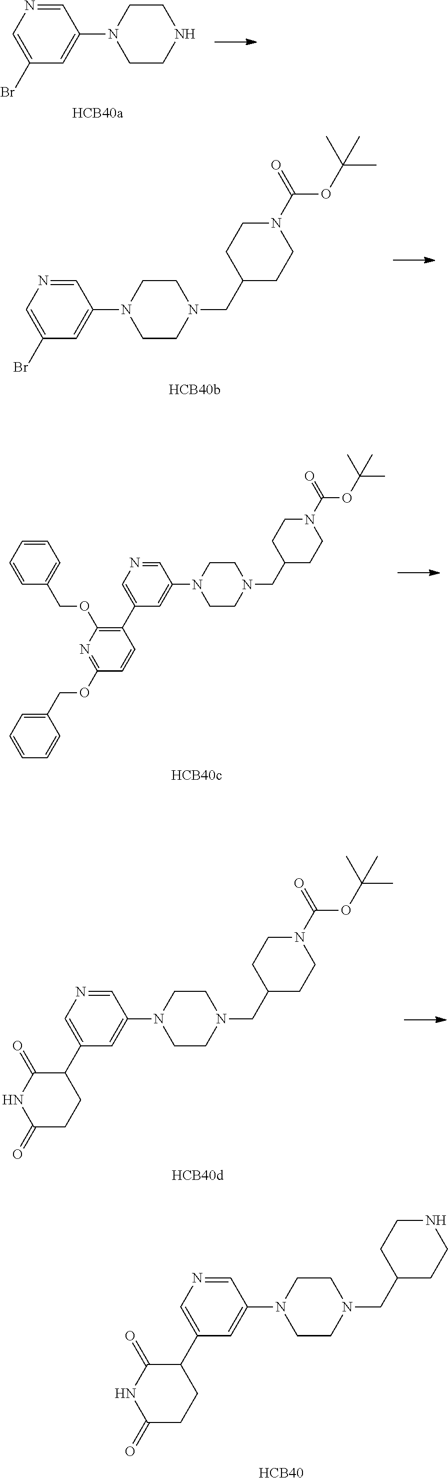

Step 1: To a solution of tert-butyl 4-formylpiperidine-1I-carboxylate (154.15 mg, 0.72 mmol) and 1-(5-bromopyridin-3-yl)piperazine (175.00 mg, 0.72 mmol) in DCM (5 ml), was added trientylamine (73 mg, 0.72 mmol) and sodium triacetoxyborohydride (0.46 g, 2.17 mmol), stirred for 30 minutes. The reaction was quenched with sodium bicarbonate solution (2 mL), and the mixture was extracted DCM (50 mL). The organic layer was dried over Na₂SO₄, filtered, and concentrated. Silica gel column purification eluting with 50-100% EtOAc in DCM provided desired product (251 mg, 0.57 mmol, 78%). tert-butyl 4-((4-(5-bromopyridin-3-yl)piperazin-1-yl)methyl)piperidine-1-carboxylate. LCMS: C₂₀H₃₁BrN₄O₂ requires: 439.2, found: m/z=441.3 [M+H]⁺.

Step 2: To a solution of tert-butyl 4-{[4-(5-bromopyridin-3-yl)piperazin-1-yl]methyl}piperidine-1-carboxylate (172.0 mg, 0.39 mmol), 2,6-bis(benzyloxy)pyridin-3-ylboronic acid (131.2 mg, 0.39 mmol) and Pd(dppf)Cl2-DCM (63.93 mg, 0.08 mmol) in dioxane (3.00 mL) and water (0.75 mL), was added caesium carbonate (382.62 mg, 1.17 mmol), the solution was bubbled in nitrogen gas and heated at 100° C. for 2 hours. The reaction was cooled, diluted with EtOAc (40 mL), and washed with brine. The organic layer was dried over Na₂SO₄, filtered, and concentrated. Silica gel column purification eluting with EtOAc in DCM (60-100%) afforeded desired product (132 mg, 0.2 mmol, 52%). tert-butyl 4-((4-(2′,6′-bis(benzyloxy)-[3,3′-bipyridin]-5-yl)piperazin-1-yl)methyl)piperidine-1-carboxylate. LCMS: C₃₉H₄₇N₅O₄ requires: 649.4, found: m/z=650.7 [M+H]⁺.

Step 3: A suspension of tert-butyl 4-((4-(2′,6′-bis(benzyloxy)-[3,3′-bipyridin]-5-yl)piperazin-1-yl)methyl)piperidine-1-carboxylate (130 mg, 0.20 mmol,) and Pd on carbon (40 mg) in ethanol (3 mL) and THE (3 mL) was stirred under hydrogen gas overnight. The solids were filtered and the volatiles were removed. Silica gel column purification eluting with MeOH in DCM (0-8%), provided the desired product (10 mg, 0.02 mmol, 10.6%). tert-butyl 4-((4-(5-(2,6-dioxopiperidin-3-yl)pyridin-3-yl)piperazin-1-yl)methyl)piperidine-1-carboxylate. LCMS: C₂₅H₃₇N₅O₄ requires: 471.3, found: m/z=472.5 [M+H]⁺.

Step 4: tert-butyl 4-((4-(5-(2,6-dioxopiperidin-3-yl)pyridin-3-yl)piperazin-1-yl)methyl)piperidine-1-carboxylate (10 mg, 0.02 mmol) was dissolved in 1 ml 4N HCl in dioxane, and the reaction was left to stand for 30 minutes. After the volatiles were removed the desired product was obtained (quantitative yield). 3-(5-(4-(piperidin-4-ylmethyl)piperazin-1-yl)pyridin-3-yl)piperidine-2,6-dione carboxylate. LCMS: C₂₀H₂₉N₅O₂ requires: 371.2, found: m/z=372.4 Example 45. 3-(6-(4-((methyl(piperidin-4-yl)amino)methyl)piperidin-1-yl)pyridin-3-yl)piperidine-2,6-dione (HCB41)

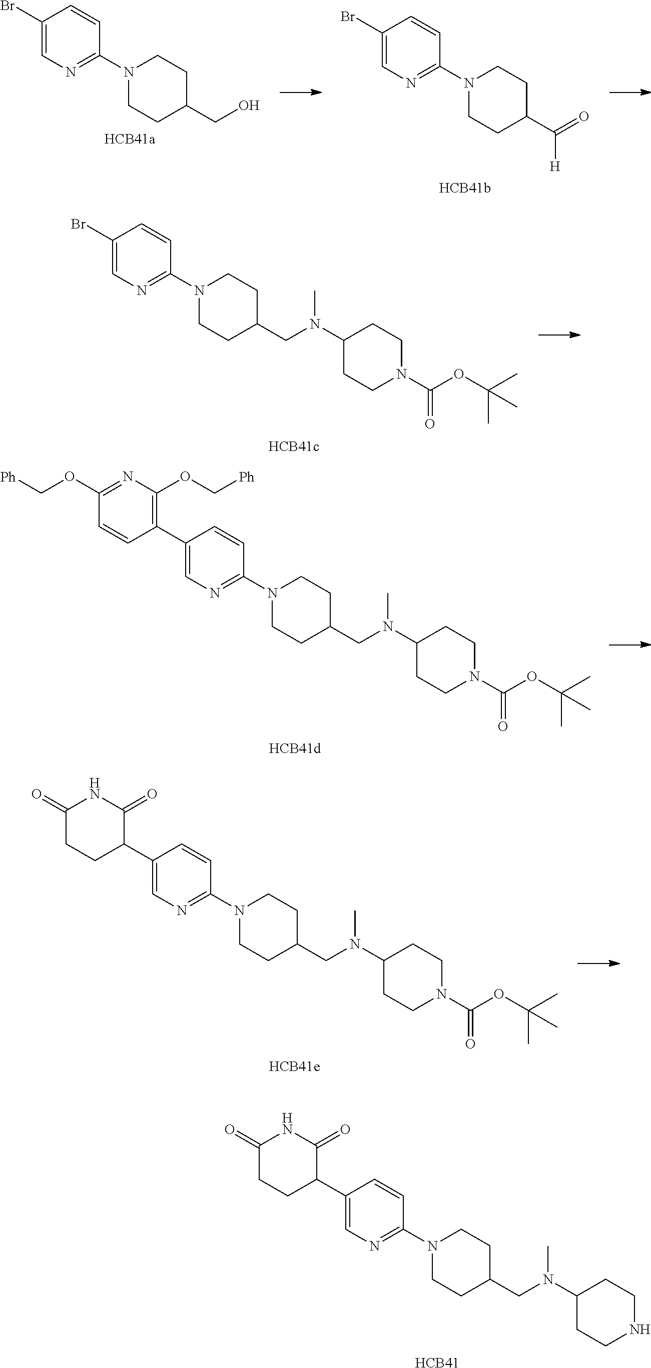

Step 1 and 2: [1-(5-bromopyridin-2-yl)piperidin-4-yl]methanol (150 mg, 0.55 mmol) was dissolved in DCM (4 mL) and DMSO (1 mL), and then triethylamine (0.77 mL, 5.53 mmol) and sulfur trioxide pyridine complex (440 mg, 2.77 mmol) was added. The reaction was stirred for 30 min when TLC indicated no more starting material remained. The reaction mixture was diluted with DCM (40 mL), and the organic solution was washed with water (2 mL×2). The organic solution was dried over sodium sulfate, concentrated, and the crude product was then re-dissolved in DCM (10 mL).Tert-butyl 4-(methylamino)piperidine-1-carboxylate (119 mg, 0.55 mmol) and sodium triacetoxyborohydride (0.3 g, 1.5 mmol) was added, and the reaction was stirred for thirty minutes. The reaction was quenched with sodium bicarbonate solution (2 mL) and extracted with DCM (50 mL). The organic layer was dried over Na₂SO₄, filtered, and concentrated. Silica gel column purification eluting with 0-6% MeOH in DCM provided the desired product (75 mg, 0.16 mmol, 29% over two steps). LCMS: C₂₂H₃₅BrN₄O₂ requires: 467, found: m/z=469 [M+H]⁺.

Step 3: 2,6-bis(benzyloxy)pyridin-3-ylboronic acid (52.34 mg, 0.16 mmol), tert-butyl 4-({[1-(5-bromopyridin-2-yl)piperidin-4-yl]methyl}(methyl)amino)piperidine-1-carboxylate (73.00 mg, 0.16 mmol) and caesium carbonate (152.65 mg, 0.47 mmol), Pd(dppf)C₁₂-DCM (25.51 mg, 0.03 mmol) were dissolved in 2 ml dioxane and 0.5 ml water, bubbled nitrogen gas for 3 minutes. The reaction mixture was heated at 100° C. for 1 hour. LCMS showed no starting material left. Evaporated solvent under reduced pressure. Dissolved product in 30 ml EtOAc, washed with brine, dried, concentrated. ISCO silica gel column purification eluting with MeOH/DCM (0-10%), isolated tert-butyl 4-[({1-[2′,6′-bis(benzyloxy)-[3,3′-bipyridin]-6-yl]piperidin-4-yl}methyl)(methyl)amino]piperidine-1-carboxylate carboxylate (68 mg, 64%). LCMS: C₄₁H₅₁N₅O₄ requires: 677.4, found: m/z=678.6 [M+H]⁺.

Step 4: The suspension of tert-butyl 4-[({1-[2′,6′-bis(benzyloxy)-[3,3′-bipyridin]-6-yl]piperidin-4-yl}methyl)(methyl)amino]piperidine-1-carboxylate (68.00 mg, 0.10 mmol) and 20 mg palldium on carbon (wet) in 5 ml ethanol was stirred under hydrogen gas overnight. The solids were filtered and the volatiles were removed. Silica gel column purification eluting with MeOH:DCM with 2% TEA) (0-10%), provided the desired product (28 mg, 0.06 mmol, 56%). tert-butyl 4-[({1-[5-(2,6-dioxopiperidin-3-yl)pyridin-2-yl]piperidin-4-yl}methyl)(methyl)amino]piperidine-1-carboxylate. LCMS: C₂₇H₄₁N₅O₄ requires: 499.3, found: m/z=500.5 [M+H]⁺.

Step 5: 4-[({1-[5-(2,6-dioxopiperidin-3-yl)pyridin-2-yl]piperidin-4-yl}methyl)(methyl)amino]piperidine-1-carboxylate (28 mg, 0.06 mmol) was dissolved in DCM (1 mL). 4 N HCl in dioxane (1 mL) was added and the reaction was left to stand for one hour. After the volatiles were removed the desired product was obtained (quantitative yield). 3-(6-(4-((methyl(piperidin-4-yl)amino)methyl)piperidin-1-yl)pyridin-3-yl)piperidine-2,6-dione. LCMS: C₂₂H₃₃N₅O₄ requires: 399.3, found: m/z=400.5 [M+H]⁺.

### Example 46. 3-(6-(6-(piperidin-4-ylmethyl)-2,6-diazaspiro[3.4]octan-2-yl)pyridin-3-yl)piperidine-2,6-dione (HCB42)

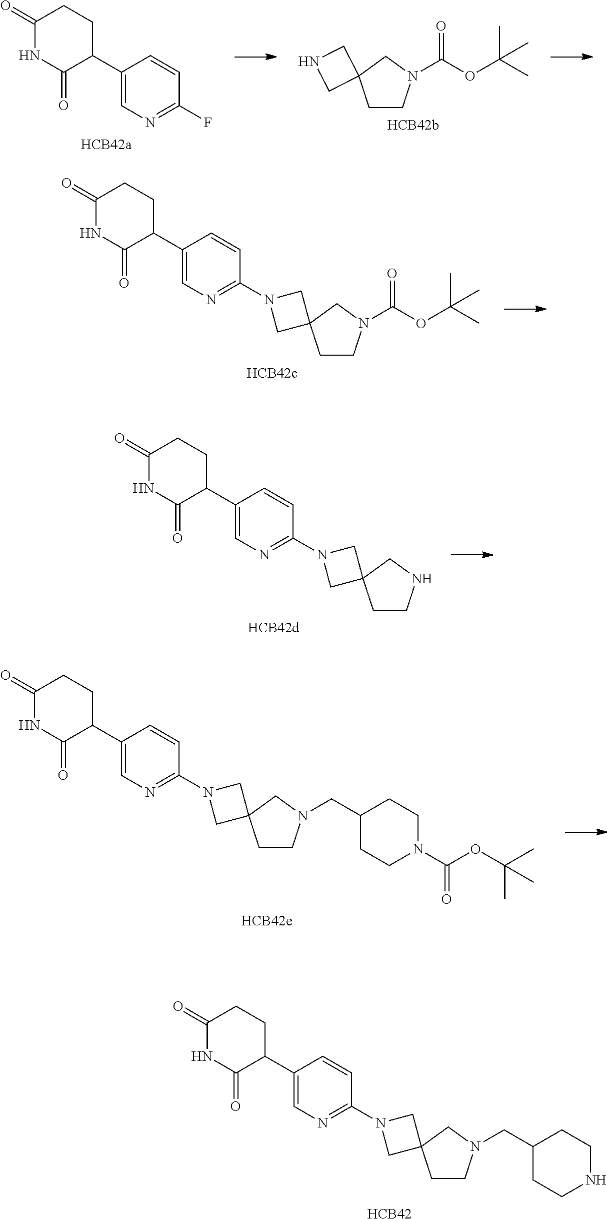

Step 1: A mixture of 3-(6-fluoropyridin-3-yl)piperidine-2,6-dione (28 mg, 0.13 mmol), tert-butyl 2,6-diazaspiro[3.4]octane-6-carboxylate (31 mg, 0.15 mmol), NMP (0.33 mL), and i-Pr₂NEt (93.7 μL, 0.07 g, 0.54 mmol) was stirred at 150° C. for one hour. The reaction was diluted with EtOAc (5 mL) and H₂O (10 mL) and the organic layer was concentrated under reduced pressure. Reverse phase flash chromatography (25-70% MeCN:H₂O with 0.1% TFA) provided the desired compound. LCMS: C₂₁H₂₈N₄O₄ requires: 400, found: m/z=401 [M+H]⁺.

Step 2: tert-butyl 2-[5-(2,6-dioxopiperidin-3-yl)pyridin-2-yl]-2,6-diazaspiro[3.4]octane-6-carboxylate (54 mg, 0.13 mmol) was dissolved in DCM (0.6 mL) and then TFA (387 μL, 0.61 g, 5.39 mmol) was added. After 20 min the volatiles were removed under reduced pressure. LCMS: C₁₆H₂₀N₄O₂ requires: 300.2, found: m/z=301.6 [M+H]⁺.

Step 3: A mixture of 3-(6-(2,6-diazaspiro[3.4]octan-2-yl)pyridin-3-yl)piperidine-2,6-dione (45 mg, 0.14 mmol), DCM (1.15 mL), and tert-butyl 4-formylpiperidine-1-carboxylate (33.6 mg, 0.16 mmol) was treated with NaBH(OAc)₃ (45 mg, 0.21 mmol). After one hour, the reaction was diluted with DCM (5 mL) and saturated aqueous NaHCO₃ (10 mL), and the organic phase was concentrated under reduced pressure to give the desired product (69 mg, 99%) which was used in next step without further manipulation. LCMS: C₂₇H₃₉N₅O₄ requires: 497.3, found: m/z=498.5 [M+H]⁺.

Step 4: A mixture of 3-(6-(6-(piperidin-4-ylmethyl)-2,6-diazaspiro[3.4]octan-2-yl)pyridin-3-yl)piperidine-2,6-dione (69 mg, 0.14 mmol) in DCM (0.47 mL) was treated with TFA (470 μL, 6.14 mmol) and the reaction was stirred for 20 min. The volatiles were removed under reduced pressure and the crude material was placed under vacuum to give the desired product (0.125 g, 0.157 mmol), which was used in the next step without further purification. LCMS: C₂₂H₃₁N₅O₂ requires: 397.3, found: m/z=398.6 [M+H]⁺.

### Example 47. 3-(6-(4-(hydroxymethyl)piperidin-1-yl)pyridin-3-yl)piperidine-2,6-dione (HCB43)

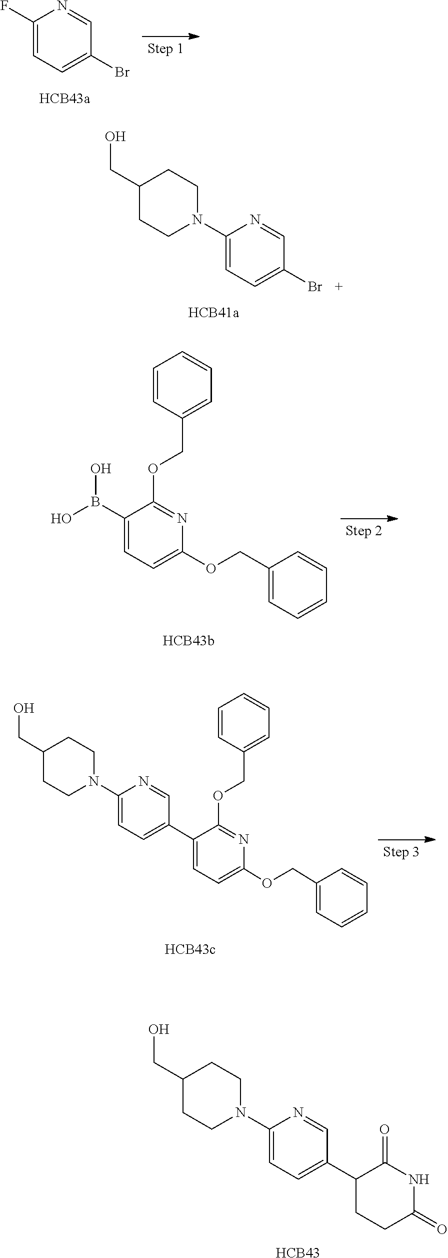 [SMILES: Fc1ccc(Br)cn1 ; OCC1CCN(c2ccc(Br)cn2)CC1 ; OB(O)c1ccc(OCc2ccccc2)nc1OCc1ccccc1 ; OCC1CCN(c2ccc(-c3ccc(OCc4ccccc4)nc3OCc3ccccc3)cn2)CC1 ; "HCB43" ; O=C1CCC(c2ccc(N3CCC(CO)CC3)nc2)C(=O)N1]

### Step 1: Synthesis of [1-(5-bromopyridin-2-yl)piperidin-4-yl]methanol (HCB41a)

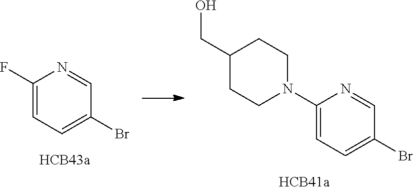 [SMILES: Fc1ccc(Br)cn1 ; OCC1CCN(c2ccc(Br)cn2)CC1]

To a mixture of 5-bromo-2-fluoropyridine (1.06 g, 6.00 mmol) in DMF (13.00 mL) was added piperidin-4-ylmethanol (691.10 mg, 6.00 mmol) and potassium carbonate (1.66 g, 12.00 mmol). The mixture was heated at 90° C. overnight. The mixture was partitioned between ethyl acetate and water, and the organic layer was dried over Na₂SO₄, filtered, and concentrated. The crude material was purified by flash chromatography on a 40 g column eluted with zero to 10% MeOH:DCM to provide [1-(5-bromopyridin-2-yl)piperidin-4-yl]methanol (829 mg, 51.7%). LCMS: CnH₁₅BrN₂O requires 270, found: m/z=271 [M+H]⁺.

### Step 2: Synthesis of {1-[2′,6′-bis(benzyloxy)-[3,3′-bipyridin]-6-yl]piperidin-4-yl}methanol (HCB43c)

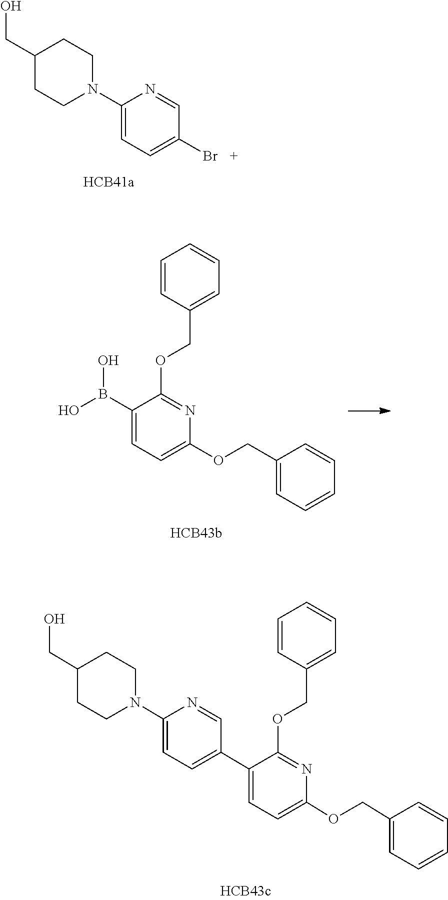 [SMILES: OCC1CCN(c2ccc(Br)cn2)CC1 ; OB(O)c1ccc(OCc2ccccc2)nc1OCc1ccccc1 ; OCC1CCN(c2ccc(-c3ccc(OCc4ccccc4)nc3OCc3ccccc3)cn2)CC1]

[1-(5-bromopyridin-2-yl)piperidin-4-yl]methanol (282 mg, 1.04 mmol), 2,6-bis(benzyloxy)pyridin-3-ylboronic acid (523 mg, 1.56 mmol), tetrakis(triphenylphosphine)palladium(0) (120 mg, 0.10 mmol), and potassium carbonate (287 mg, 2.08 mmol) were deposited in a microwave vial in THE (3.00 mL) and water (1.00 mL). The mixture was microwaved at 120° C. for forty minutes. The organic layer was loaded directly onto a silica gel cartridge and the mixture was purified by flash chromatography on a 40 g column (gradient elution with zero to 50% EtOAc:DCM) to provide {1-[2′,6′-bis(benzyloxy)-[3,3′-bipyridin]-6-yl]piperidin-4-yl}methanol (0.198 g, 39.5%). LCMS: C₃₀H₃₁N₃O₃ requires 481, found: m/z=482 [M+H]⁺.

### Step 3: Synthesis of 3-(6-(4-(hydroxymethyl)piperidin-1-yl)pyridin-3-yl)piperidine-2,6-dione (HCB43)

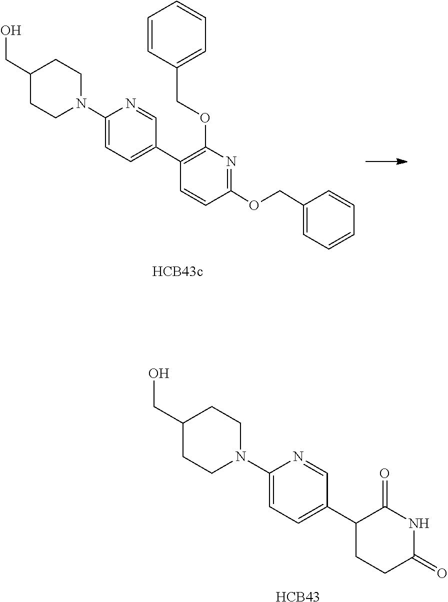 [SMILES: OCC1CCN(c2ccc(-c3ccc(OCc4ccccc4)nc3OCc3ccccc3)cn2)CC1 ; "HCB43" ; O=C1CCC(c2ccc(N3CCC(CO)CC3)nc2)C(=O)N1]

{1-[2′,6′-bis(benzyloxy)-[3,3′-bipyridin]-6-yl]piperidin-4-yl}methanol (198 mg, 0.41 mmol) and 10% Pd/C (198 mg) were stirred in EtOH (6.00 mL) under a balloon of H₂ for five hours. The mixture was diluted with THF and filtered through celite. The mixture was concentrated in vacuo and purified by flash chromatography on a 12 g column (gradient elution with zero to 20% MeOH:DCM) to provide 3-(6-(4-(hydroxymethyl)piperidin-1-yl)pyridin-3-yl)piperidine-2,6-dione (0.0339 g, 27.2%). LCMS: C₁₆H₂₁N₃O₃ requires 303, found: m/z=304 [M+H]⁺.

### Example 48. 3-(2-(4-(hydroxymethyl)piperidin-1-yl)pyridin-4-yl)piperidine-2,6-dione (HCB44)

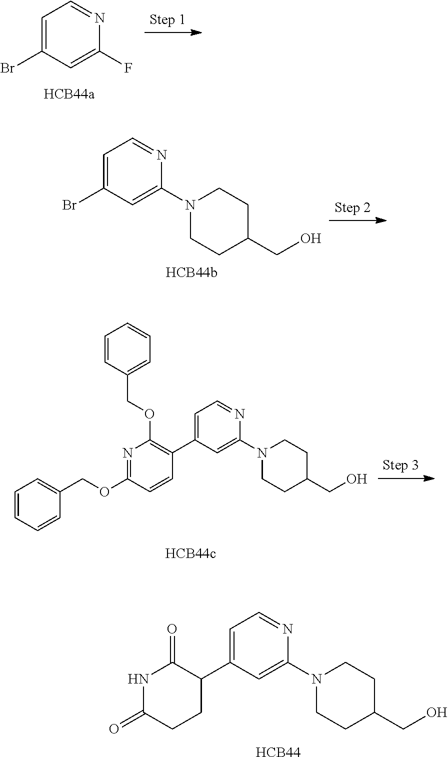 [SMILES: Fc1cc(Br)ccn1 ; OCC1CCN(c2cc(Br)ccn2)CC1 ; OCC1CCN(c2cc(-c3ccc(OCc4ccccc4)nc3OCc3ccccc3)ccn2)CC1 ; "HCB44" ; O=C1CCC(c2ccnc(N3CCC(CO)CC3)c2)C(=O)N1]

### Step 1: [1-(4-bromopyridin-2-yl)piperidin-4-yl]methanol (HCB44b)

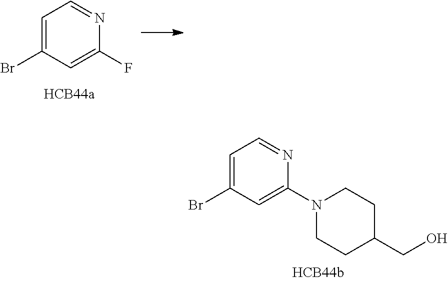 [SMILES: Fc1cc(Br)ccn1 ; OCC1CCN(c2cc(Br)ccn2)CC1]

4-bromo-2-fluoropyridine (1.02 g, 5.78 mmol), potassium carbonate (2.40 g, 17.3 mmol), and piperidin-4-ylmethanol (798 mg, 6.93 mmol) in DMSO (14.00 mL) was heated at 100° C. for one hour. The mixture was poured into water and then extracted with ethyl acetate. The organic layer was dried over Na₂SO₄, filtered, and concentrated in vacuo. The crude residue was purified by flash chromatography on a 40 g column (gradient elution with zero to 50% ethyl acetate:DCM to provide [1-(4-bromopyridin-2-yl)piperidin-4-yl]methanol (1.3200 g, 84.2%). LCMS: CnH₁₅BrN₂O requires: 270, found: m/z=271 [M+H]⁺.

### Step 2: {1-[2,6-bis(benzyloxy)-[3,4′-bipyridin]-2′-yl]piperidin-4-yl}methanol (HCB44c)

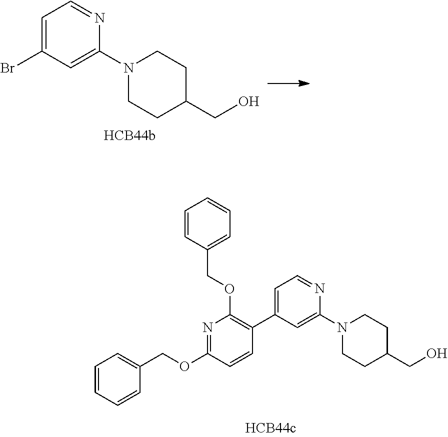 [SMILES: OCC1CCN(c2cc(Br)ccn2)CC1 ; OCC1CCN(c2cc(-c3ccc(OCc4ccccc4)nc3OCc3ccccc3)ccn2)CC1]

A mixture of [1-(4-bromopyridin-2-yl)piperidin-4-yl]methanol (402 mg, 1.48 mmol), 2,6-bis(benzyloxy)pyridin-3-ylboronic acid (497 mg, 1.48 mmol), tetrakis(triphenylphosphine)palladium(0) (171 mg, 0.15 mmol), and potassium carbonate (410 mg, 2.97 mmol) in THF (3.00 mL) and water (1.00 mL) was microwaved at 120° C. for forty minutes. The water layer was removed and the organic layer was concentrated. The crude residue was purified by flash chromatography on a 40 g column (gradient elution with zero to 100% ethyl acetate:DCM) to provide {1-[2,6-bis(benzyloxy)-[3,4′-bipyridin]-2′-yl]piperidin-4-yl}methanol (0.3150 g, 44.1%). LCMS: C₃₀H₃₁N₃O₃ requires: 481, found: m/z=482 [M+H]⁺.

### Step 3: 3-(2-(4-(hydroxymethyl)piperidin-1-yl)pyridin-4-yl)piperidine-2,6-dione (HCB44)

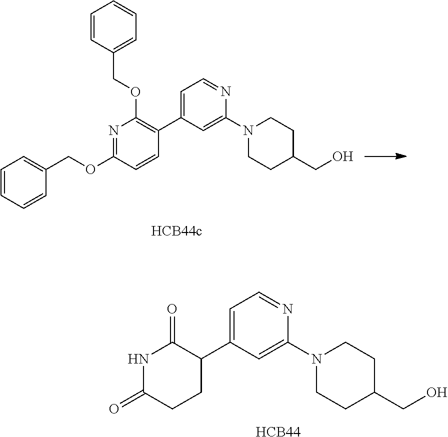 [SMILES: OCC1CCN(c2cc(-c3ccc(OCc4ccccc4)nc3OCc3ccccc3)ccn2)CC1 ; "HCB44" ; O=C1CCC(c2ccnc(N3CCC(CO)CC3)c2)C(=O)N1]

{1-[2,6-bis(benzyloxy)-[3,4′-bipyridin]-2′-yl]piperidin-4-yl}methanol (315 mg, 0.65 mmol) and 10% Pd/C (315.00 mg) were stirred in EtOH (6.00 mL) and THE (3.00 mL) under a balloon of H₂ for four hours. The mixture was diluted with THF (100 mL) and filtered through celite. The celite pad was washed with MeOH and DCM and the resulting solution was concentrated. The crude residue was purified by flash chromatography on a 24 g column (gradient elution with zero to 10% MeOH:DCM) to provide 3-(2-(4-(hydroxymethyl)piperidin-1-yl)pyridin-4-yl)piperidine-2,6-dione (0.0729 g, 36.7%). LCMS: C₁₆H₂₁N₃O₃ requires: 303, found: m/z=304 [M+H]⁺.

### Example 49. (3R)-1-(1-(2,6-dioxopiperidin-3-yl)-3-methyl-2-oxo-2,3-dihydro-1H-benzo[d]imidazol-5-yl)pyrrolidine-3-carbaldehyde (HCB45)

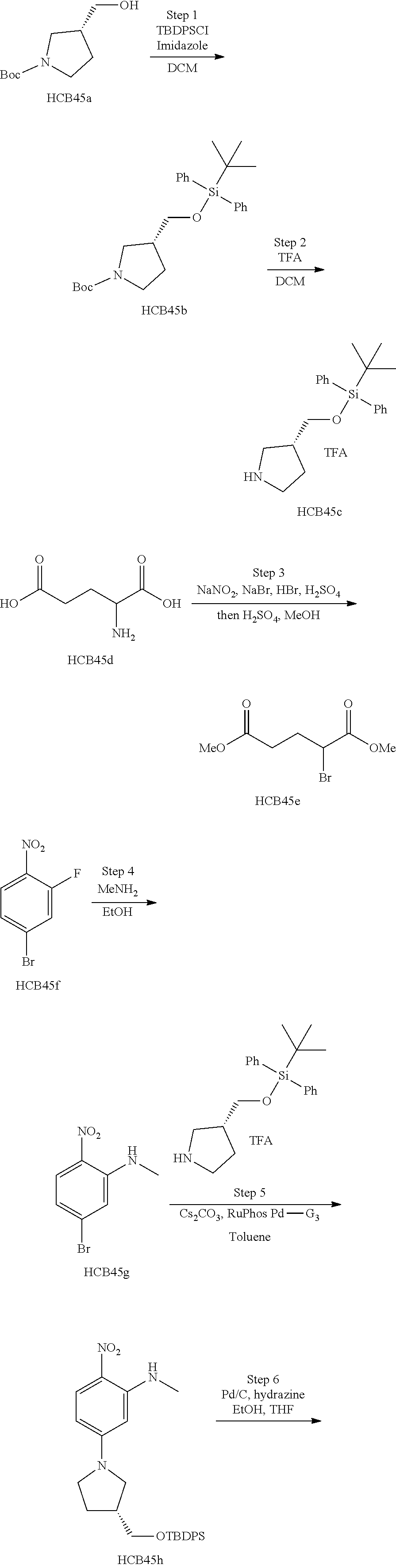

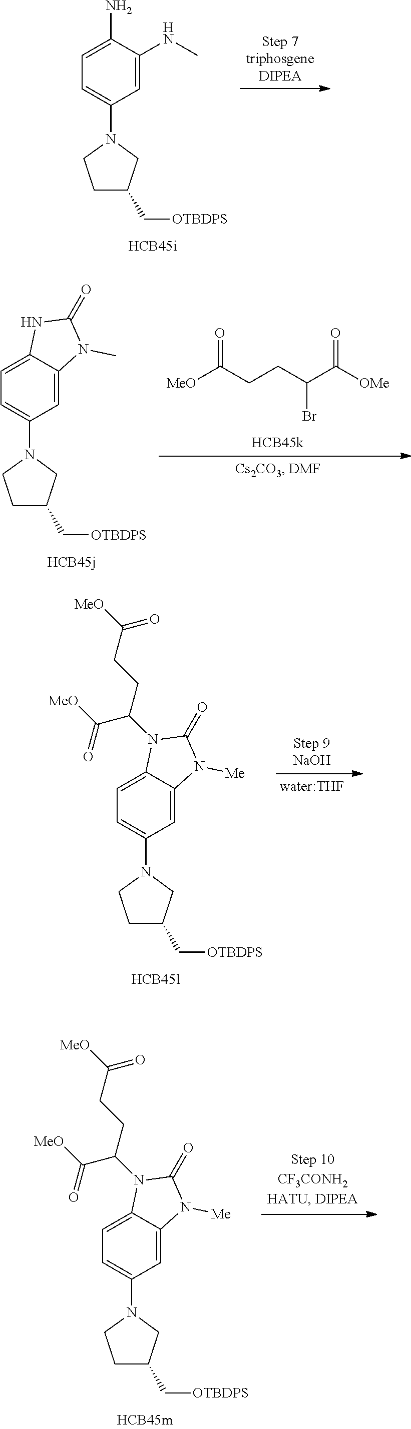

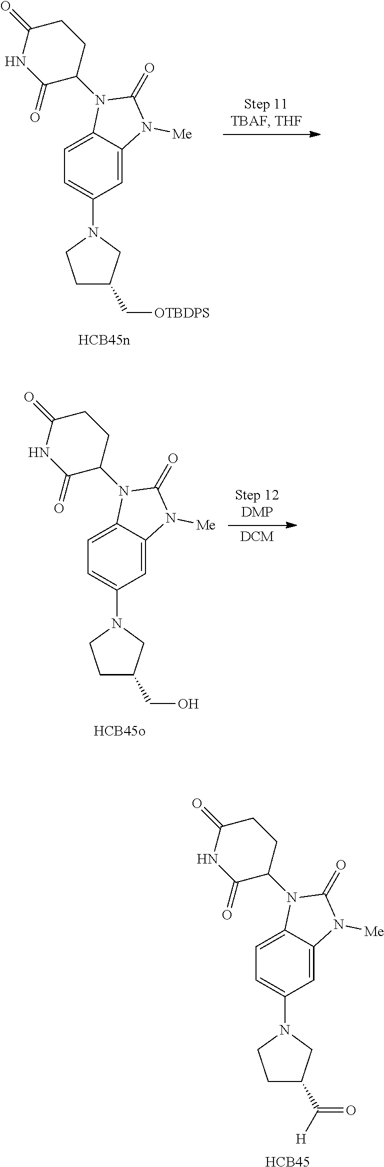

### Step 1: tert-butyl (3R)-3-[[tert-butyl(diphenyl)silyl]oxymethyl]pyrrolidine-1-carboxylate (HCB45b)

TBDPSCI (32.3 mL, 124 mmol) was added to a mixture of tert-butyl (3R)-3-(hydroxymethyl)pyrrolidine-1-carboxylate (25.0 g, 124 mmol) and imidazole (10.1 g, 149 mmol) in DCM (500 mL) at 0° C. under nitrogen. The mixture was stirred at 23° C. for 16 h and diluted with water (300 mL). The organic phase was washed with water (100 mL), brine (3×100 mL), dried (Na₂SO₄), filtered, and concentrated to provide the title product as an oil (54.0 g, 99%). m/z: ES⁺[M-C₆H₅-tBu+H]⁺=306.2, LCMS (A05); Rt=2.47 min. ¹H NMR (500 MHz, CDCl₃) δ 7.67-7.60 (m, 4H), 7.46-7.34 (m, 6H), 3.64-3.55 (m, 2H), 3.45-3.37 (m, 1H), 3.37-3.22 (m, 1H), 3.17-3.07 (m, 1H), 2.42 (m, 1H), 1.97-1.86 (m, 1H), 1.74-1.62 (m, 1H), 1.60 (s, 1H), 1.46 (s, 9H), 1.08-1.02 (m, 9H).

### Step 2: tert-butyl-diphenyl-[[(3R)-pyrrolidin-3-yl]methoxy]silane 2,2,2-trifluoroacetic acid (HCB45c)

TFA (50 mL) was added to a mixture of tert-butyl (3R)-3-[[tert-butyl(diphenyl)silyl]oxymethyl]pyrrolidine-1-carboxylate (54.0 g, 123 mmol) in DCM (200 mL) at 23° C. under nitrogen. The mixture was stirred at 23° C. for 1.5 h and concentrated. The residue was diluted with PhMe (150 mL) and concentrated (process repeated twice) to provide the title compound as an oil (55.7 g, quant.). m/z: ES⁺[M+H-TFA]+=340.3, LCMS (A05); Rt=2.32 min. ¹H NMR (500 MHz, CDCl₃) δ 8.82 (s, 2H), 7.65-7.57 (m, 4H), 7.52-7.40 (m, 6H), 3.65 (d, J=6.4 Hz, 2H), 3.35-3.27 (m, 1H), 3.24-3.10 (m, 2H), 3.01-2.91 (m, 1H), 2.58-2.52 (m, 1H), 2.04-1.93 (m, 1H), 1.74-1.63 (m, 1H), 1.01 (s, 9H).

### Step 3: dimethyl 2-bromopentanedioate acid (HCB45e)

A solution of NaNO₂ (25.5 g, 370 mmol) in water (50 mL) was added to a mixture of (2S)-2-aminopentanedioic acid (30 g, 204 mmol), NaBr (73.2 g, 711 mol), and HBr (50 mL, 48% in water) in water (100 mL) at 0° C. (keeping the internal temperature below 10° C.) under nitrogen. The mixture was stirred at 23° C. for 6 h and then H₂SO₄ (25.0 mL) was added at 23° C. The mixture was extracted with Et₂O (4×70.0 mL) and the combined organic phases were washed with brine (2×50.0 mL), dried (Na₂SO₄), filtered, and concentrated. H₂SO₄ (10.0 mL) was added to the mixture of the residue in MeOH (80.0 mL) at 23° C. under nitrogen. The mixture was refluxed for 16 h, cooled to 23° C., and concentrated. The residue was diluted with Et₂O (100 mL) and water (100 mL). The aq. phase was extracted with Et₂O (4×50.0 mL). The combined organic layers were washed with water (60.0 mL), NaHCO₃ (2×60.0 mL), brine (2×50.0 mL), dried (Na₂SO₄), filtered, and concentrated to provide the title compound as an oil (19 g, 39%). ¹H NMR (400 MHz, CDCl₃) δ 4.34 (dd, J=8.5, 5.8 Hz, 1H), 3.75 (s, 3H), 3.65 (s, 3H), 2.52-2.45 (m, 2H), 2.40-2.30 (m, 1H), 2.26 (m, 1H).

### Step 4: 5-bromo-N-methyl-2-nitro-aniline (HCB45 g)

Methylamine (56.6 mL, 455 mmol, 33% wt in EtOH) was added to a mixture of 4-bromo-2-fluoro-1-nitro-benzene (50.0 g, 227 mmol) in EtOH (455 mL) at 23° C. under nitrogen. The mixture was stirred at 23° C. for 30 min, filtered, and washed with cold EtOH (200 mL) to provide the title compound as a solid (48.2 g, 92%). m/z (ES⁺) [M+H]⁺=231.0, LCMS (A05); Rt=2.51 min. ¹H NMR (400 MHz, DMSO-d₆) δ 8.23 (d, J=4.3 Hz, 1H), 7.98 (d, J=9.1 Hz, 1H), 7.17 (d, J=2.0 Hz, 1H), 6.82 (dd, J=9.1, 2.1 Hz, 1H), 2.95 (d, J=5.0 Hz, 3H).

### Step 5: (R)-5-(3-(((tert-butyldiphenylsilyl)oxy)methyl)pyrrolidin-1-yl)-N-methyl-2-nitroaniline (HCB45 h)

RuPhos-Pd-G₃ (2.71 g, 3.25 mmol) was added to a mixture of 5-bromo-N-methyl-2-nitro-aniline, (25 g, 108 mmol), tert-butyl-diphenyl-[[(3R)-pyrrolidin-3-yl]methoxy]silane 2,2,2-trifluoroacetic acid (60.0 g, 119 mmol, 90% purity), and Cs₂CO₃ (106 g, 325 mmol) in PhMe (600 mL) at 23° C. under nitrogen. The mixture was degassed by bubbling nitrogen for 15 min at 23° C., then stirred at 100° C. for 19 h, cooled to 23° C., filtered, and concentrated. The product was purified by silica gel chromatography (2×330 g in-series cartridges) with hexanes and EtOAc (0-50%) to provide the title compound as a solid (41.0 g, 77%). m/z: ES⁺[M+H]=490.4. ¹H NMR (400 MHz, DMSO-d₆) δ 8.36 (d, J=4.9 Hz, 1H), 7.91 (d, J=9.6 Hz, 1H), 7.64-7.57 (m, 4H), 7.50-7.37 (m, 6H), 6.07 (dd, J=9.6, 2.5 Hz, 1H), 5.50 (d, J=2.4 Hz, 1H), 3.68 (d, J=6.6 Hz, 2H), 3.54-3.46 (m, 1H), 3.46-3.37 (m, 2H), 3.27-3.21 (m, 1H), 2.90 (d, J=5.0 Hz, 3H), 2.64-2.55 (m, 1H), 2.16-2.04 (m, 1H), 1.90-1.79 (m, 1H), 1.01 (s, 9H).

### Step 6: (R)-5-(3-(((tert-butyldiphenylsilyl)oxy)methyl)pyrrolidin-1-yl)-N₁-methylbenzene-1,2-diamine (HCB45i)

A solution of (R)-5-(3-(((tert-butyldiphenylsilyl)oxy)methyl)pyrrolidin-1-yl)-N-methyl-2-nitroaniline, (20.0 g, 40.8 mmol) in THE (100 mL) and EtOH (100 mL) was added to 10% Pd/C (4.4 g, 4.1 mmol, 50% wet.) at 23° C. under nitrogen. The mixture was refluxed and then hydrazine hydrate (16 mL, 163 mmol) was added over 30 min. The mixture was refluxed for 2 h, cooled to 23° C., filtered (Celite), washed with EtOAc (200 mL) and EtOH (200 mL), and concentrated to provide the title compound as an oil (18.0 g, 96%). m/z ESI⁺[M-Ph-tBu+H]⁺=328.16. ¹H NMR (400 MHz, DMSO-d₆) δ 7.65-7.59 (m, 4H), 7.48-7.38 (m, 6H), 6.45-6.40 (m, 1H), 5.71 (d, J=2.5 Hz, 1H), 5.66 (dd, J=8.1, 2.5 Hz, 1H), 4.50 (d, J=4.9 Hz, 1H), 3.65 (d, J=6.8 Hz, 2H), 3.33 (br s, 2H), 3.21 (dd, J=9.1, 7.6 Hz, 1H), 3.14-3.07 (m, 2H), 2.97 (dd, J=9.2, 5.9 Hz, 1H), 2.68 (d, J=4.2 Hz, 3H), 2.56-2.51 (m, 1H), 2.05-1.95 (m, 1H), 1.76-1.67 (m, 1H), 1.01 (s, 9H).

### Step 7: (R)-6-(3-(((tert-butyldiphenylsilyl)oxy)methyl)pyrrolidin-1-yl)-1-methyl-1,3-dihydro-2H-benzo[d]imidazol-2-one (HCB45j)

A mixture of triphosgene (8.09 g, 27.3 mmol) in DCM (30 mL) was added to a mixture of (R)-5-(3-(((tert-butyldiphenylsilyl)oxy)methyl)pyrrolidin-1-yl)-N₁-methylbenzene-1,2-diamine (38.0 g, 82.7 mmol) and DIPEA (115 mL, 661 mmol) in DCM (300 mL) at 0° C. under nitrogen. The mixture was stirred at 0° C. for 30 min and diluted with water (300 mL). The aqueous phase was extracted with DCM (2×100 mL), and the combined organic phases were washed with brine (50.0 mL), dried (MgSO₄), filtered, and concentrated. The product was purified by silica gel chromatography (2×330 g cartridge) with DCM and MeOH (0-10%) to provide the title compound as a solid (21 g, 52%). m/z: ES⁺[M+H]=486.4,

### Step 8: 2-(5-((R)-3-(((tert-butyldiphenylsilyl)oxy)methyl)pyrrolidin-1-yl)-3-methyl-2-oxo-2,3-dihydro-1H-benzo[d]imidazol-1-yl)pentanedioate (HCB451)

Dimethyl 2-bromopentanedioate (10.9 g, 30.9 mmol, 68% purity) was added to a mixture of (R)-6-(3-(((tert-butyldiphenylsilyl)oxy)methyl)pyrrolidin-1-yl)-1-methyl-1,3-dihydro-2H-benzo[d]imidazol-2-one, (10.0 g, 20.6 mmol) and Cs₂CO₃ (20.3 g, 62.3 mmol) in DMF (100 mL) at 23° C. under nitrogen. The mixture was stirred at 100° C. for 18 h, cooled to 23° C. and diluted with EtOAc (200 mL) and water (100 mL). The aqueous phase was extracted with EtOAc (2×100 mL), and the combined organic phases were washed with brine (2×50 mL), dried (MgSO₄), filtered, and concentrated. The product was purified by silica gel chromatography (220 g cartridge) with hexanes and EtOAc (0-50%) to provide the title compound as a solid (9.00 g, 68%). m/z: ES⁺[M+H]=644.4, LCMS (A05); Rt=2.33 min.

### Step 9: 2-(5-((R)-3-(((tert-butyldiphenylsilyl)oxy)methyl)pyrrolidin-1-yl)-3-methyl-2-oxo-2,3-dihydro-1H-benzo[d]imidazol-1-yl)pentanedioic acid (HCB45m)

Aq. NaOH (5 M, 14.0 mL, 70.0 mmol) was added to a mixture of dimethyl 2-(5-((R)-3-(((tert-butyldiphenylsilyl)oxy)methyl)pyrrolidin-1-yl)-3-methyl-2-oxo-2,3-dihydro-1H-benzo[d]imidazol-1-yl)pentanedioate, (9.00 g, 14.0 mmol) in a mixture of THE and water (200 mL, 1:1 v/v) at 23° C. under nitrogen. The mixture was stirred at 23° C. for one hour and diluted with EtOAc (100 mL) and aq. HCl (1 M, 80.0 mL). The aqueous phase was extracted with EtOAc (3×50.0 mL) and the combined organic phases were washed with brine (2×50.0 mL), dried (Na₂SO₄), filtered, and concentrated to provide the title compound as a solid (8.6 g, quant.). ¹H NMR (500 MHz, DMSO-d₆) δ 7.67-7.58 (m, 4H), 7.51-7.36 (m, 6H), 6.93-6.81 (m, 1H), 6.41-6.30 (m, 1H), 6.27-6.17 (m, 1H), 4.95 (dd, J=10.8, 5.0 Hz, 1H), 3.69 (d, J=6.6 Hz, 2H), 3.38-3.31 (m, 1H), 3.29 (s, 3H), 3.27-3.19 (m, 2H), 3.12-3.03 (m, 1H), 2.65-2.55 (m, 1H), 2.41-2.21 (m, 2H), 2.21-2.02 (m, 3H), 1.86-1.79 (m, 1H), 1.02 (s, 9H).

### Step 10: 3-(5-((R)-3-(((tert-butyldiphenylsilyl)oxy)methyl)pyrrolidin-1-yl)-3-methyl-2-oxo-2,3-dihydro-1H-benzo[d]imidazol-1-yl)piperidine-2,6-dione (HCB45n)

HATU (6.792 g, 17.9 mmol) was added to a mixture of 2-(5-((R)-3-(((tert-butyldiphenylsilyl)oxy)methyl)pyrrolidin-1-yl)-3-methyl-2-oxo-2,3-dihydro-1H-benzo[d]imidazol-1-yl)pentanedioic acid, (5.0 g, 8.12 mmol), trifluoroacetamide (1.01 g, 8.93 mmol), and DIPEA (5.66 mL, 32.5 mmol) in DMF (50.0 mL) at 23° C. under nitrogen. The mixture was stirred at 23° C. for 18 h and concentrated. The product was purified by silica gel chromatography (120 g cartridge) with DCM and MeOH (0-5%) to provide the title compound as a solid (3.30 g, 68%). ¹H NMR (500 MHz, DMSO-d₆) δ 11.04 (s, 1H), 7.65-7.60 (m, 4H), 7.50-7.39 (m, 6H), 6.93-6.87 (m, 1H), 6.35 (d, J=2.1 Hz, 1H), 6.20 (dd, J=8.6, 2.2 Hz, 1H), 5.26 (dd, J=12.8, 5.4 Hz, 1H), 3.69 (d, J=6.6 Hz, 2H), 3.29 (s, 3H), 3.26-3.20 (m, 2H), 3.08-3.02 (m, 1H), 2.93-2.85 (m, 1H), 2.70 (s, 2H), 2.67-2.55 (m, 2H), 2.13-2.04 (m, 1H), 1.98-1.95 (m, 1H), 1.86-1.76 (m, 1H), 1.02 (s, 9H).

### Step 11: 3-[5-[(3R)-3-(hydroxymethyl)pyrrolidin-1-yl]-3-methyl-2-oxo-benzimidazol-1-yl]piperidine-2,6-dione (HCB45o)

TBAF (8.00 mL, 8.00 mmol, 1 M in THF) was added to a mixture of 3-(5-((R)-3-(((tert-butyldiphenylsilyl)oxy)methyl)pyrrolidin-1-yl)-3-methyl-2-oxo-2,3-dihydro-1H-benzo[d]imidazol-1-yl)piperidine-2,6-dione, (3.20 g, 5.36 mmol) in THF (20 mL) at 23° C. under nitrogen. The mixture was stirred at 23° C. for 3 h and then concentrated. The product was purified by silica gel chromatography (220 g cartridge) with DCM and MeOH (0-12%) to provide the title compound as a solid (1.30 g, 67%). m/z: ES⁺[M]⁺=358.2. ¹H NMR (400 MHz, DMSO-d₆) δ 11.03 (s, 1H), 6.89 (d, J=8.5 Hz, 1H), 6.37 (d, J=2.2 Hz, 1H), 6.21 (dd, J=8.6, 2.2 Hz, 1H), 5.25 (dd, J=12.9, 5.4 Hz, 1H), 4.69 (t, J=5.2 Hz, 1H), 3.48-3.36 (m, 2H), 3.37-3.32 (m, 1H), 3.29 (s, 3H), 3.27-3.15 (m, 2H), 3.05-2.97 (m, 1H), 2.95-2.83 (m, 1H), 2.73-2.55 (m, 2H), 2.48-2.37 (m, 1H), 2.08-1.92 (m, 2H), 1.79-1.68 (m, 1H).

### Step 12: (3R)-1-(1-(2,6-dioxopiperidin-3-yl)-3-methyl-2-oxo-2,3-dihydro-1H-benzo[d]imidazol-5-yl)pyrrolidine-3-carbaldehyde (HCB45)

To a mixture of (3RS)-3-{5-[(3R)-3-(hydroxymethyl)pyrrolidin-1-yl]-3-methyl-2-oxo-1,3-benzodiazol-1-yl}piperidine-2,6-dione (33.50 mg, 0.09 mmol) in DMSO (1.00 mL) was added triethylamine (0.26 mL, 0.19 g, 1.87 mmol) followed by sulfur trioxide pyridine complex (148.77 mg, 0.93 mmol). After 25 min, water was added and the mixture was extracted with DCM twice. The combined organic layers were concentrated to give the title compound without further purification. m/z: ES⁺[M]⁺=357.2.

### Example 50. 3-(5-(4-(hydroxymethyl)piperidin-1-yl)-3-methyl-2-oxo-2,3-dihydro-1H-benzo[d]imidazol-1-yl)piperidine-2,6-dione (HCB46)

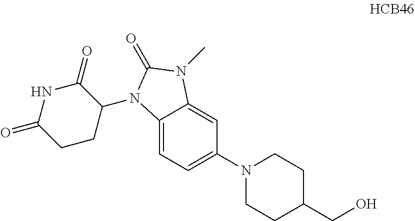 [SMILES: Cn1c(=O)n(C2CCC(=O)NC2=O)c2ccc(N3CCC(CO)CC3)cc21]

3-(5-(4-(hydroxymethyl)piperidin-1-yl)-3-methyl-2-oxo-2,3-dihydro-1H-benzo[I]imidazol-1-yl)piperidine-2,6-dione was prepared by analogous procedures in Example 48 above starting from 4-piperidinemethanol. LCMS: C₁₉H₂₄N₄O₄ requires: 372, found: m/z=373 Example 51. 3-(5-((S)-3-(hydroxymethyl)pyrrolidin-1-yl)-3-methyl-2-oxo-2,3-dihydro-1H-benzo[d]imidazol-1-yl)piperidine-2,6-dione (HCB47)

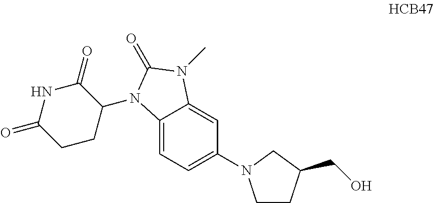 [SMILES: Cn1c(=O)n(C2CCC(=O)NC2=O)c2ccc(N3CC[C@H](CO)C3)cc21]

3-(5-((S)-3-(hydroxymethyl)pyrrolidin-1-yl)-3-methyl-2-oxo-2,3-dihydro-1H-benzo[d]imidazol-1-yl)piperidine-2,6-dione was prepared by procedures analogous to Example 48 above starting form (S)-pyrrolidin-3-ylmethanol. LCMS: C₁₈H₂₂N₄O₄ requires: 358, found: m/z=359 [M+H]⁺.

### Example 52. 3-(4-(4-(hydroxymethyl)piperidin-1-yl)-3-methyl-2-oxo-2,3-dihydro-1H-benzo[d]imidazol-1-yl)piperidine-2,6-dione (HCB48)

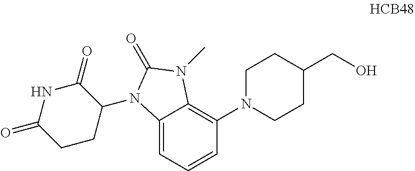 [SMILES: Cn1c(=O)n(C2CCC(=O)NC2=O)c2cccc(N3CCC(CO)CC3)c21]

3-(4-(4-(hydroxymethyl)piperidin-1-yl)-3-methyl-2-oxo-2,3-dihydro-1H-benzo[d]imidazol-1-yl)piperidine-2,6-dione was prepared by procedures analogous to Example 48 above starting from 2-bromo-N-methyl-6-nitroaniline and 4-piperidinemethanol. LCMS: C₁₉H₂₄N₄O₄ requires: 372, found: m/z=373 [M+H]⁺.

### Example 53. 1-(2,6-dioxopiperidin-3-yl)-3-methyl-2-oxo-1,3-benzodiazole-5-carbaldehyde (HCB55)

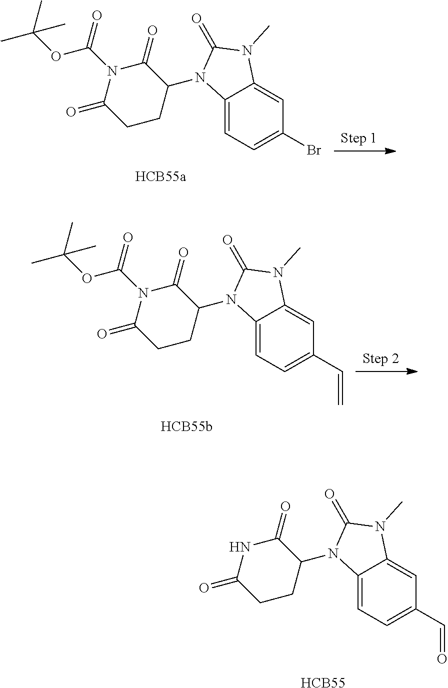 [SMILES: Cn1c(=O)n(C2CCC(=O)N(C(=O)OC(C)(C)C)C2=O)c2ccc(Br)cc21 ; C=Cc1ccc2c(c1)n(C)c(=O)n2C1CCC(=O)N(C(=O)OC(C)(C)C)C1=O ; "HCB55" ; Cn1c(=O)n(C2CCC(=O)NC2=O)c2ccc(C=O)cc21]

### Step 1: tert-butyl 3-(5-ethenyl-3-methyl-2-oxo-1,3-benzodiazol-1-yl)-2,6-dioxopiperidine-1-carboxylate (HCB55b)

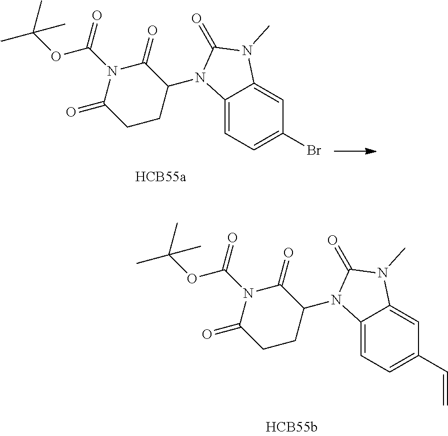 [SMILES: Cn1c(=O)n(C2CCC(=O)N(C(=O)OC(C)(C)C)C2=O)c2ccc(Br)cc21 ; C=Cc1ccc2c(c1)n(C)c(=O)n2C1CCC(=O)N(C(=O)OC(C)(C)C)C1=O]

tert-butyl 3-(5-bromo-3-methyl-2-oxo-1,3-benzodiazol-1-yl)-2,6-dioxopiperidine-1-carboxylate (70.0 mg, 0.16 mmol), potassium ethenyltrifluoroboranuide (42.8 mg, 0.32 mmol), and cesium carbonate (104 mg, 0.32 mmol) were suspended in dioxane (1.00 mL) and water (0.20 mL). A vacuum was applied and the headspace was backfilled with argon for five cycles. Then Pd(dppf)Cl₂ CH₂Cl₂ (13.0 mg, 0.02 mmol) was added. A vacuum was applied and the headspace was backfilled with argon for five cycles. The mixture was heated at 80° C. for three hours. Water was added and the mixture was extracted three times with DCM. The combined organic layers were concentrated. The crude residue was purified by flash chromatography on a 24 g column (gradient elution with zero to 10% MeOH:DCM to provide tert-butyl 3-(5-ethenyl-3-methyl-2-oxo-1,3-benzodiazol-1-yl)-2,6-dioxopiperidine-1-carboxylate (0.0470 g, 76.4%). LCMS: C₂₀H₂₃N₃O₅ requires: 385, found: m/z=408 [M+Na]⁺.

### Step 2: 1-(2,6-dioxopiperidin-3-yl)-3-methyl-2-oxo-1,3-benzodiazole-5-carbaldehyde (HCB55)

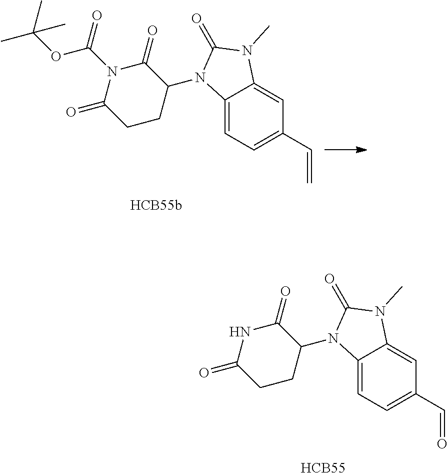 [SMILES: C=Cc1ccc2c(c1)n(C)c(=O)n2C1CCC(=O)N(C(=O)OC(C)(C)C)C1=O ; "HCB55" ; Cn1c(=O)n(C2CCC(=O)NC2=O)c2ccc(C=O)cc21]

To a mixture of tert-butyl 3-(5-ethenyl-3-methyl-2-oxo-1,3-benzodiazol-1-yl)-2,6-dioxopiperidine-1-carboxylate (47.0 mg, 0.12 mmol) in dioxane (2.50 mL) and water (0.25 mL) was added sodium metaperiodate (51.3 mg, 0.24 mmol) followed by osmium tetroxide (29.4 mg, 0.12 mmol) and methyl morpholine oxide (7.14 mg, 0.06 mmol). After thirty minutes, water was added and the mixture was extracted twice with ethyl acetate. Brine was added to get the layers to separate. The combined organic layers were dried over Na₂SO₄, filtered, and concentrated in vacuo. The crude residue was purified by flash chromatography on a 12 g column (gradient elution with zero to 10% MeOH:DCM) to provide 1-(2,6-dioxopiperidin-3-yl)-3-methyl-2-oxo-1,3-benzodiazole-5-carbaldehyde (0.0142 g, 40.5%). LCMS C₁₄H₁₃N₃O₄ requires: 287, found: m/z=288 [M+H]⁺.

### General Schemes For Preparing LHP Building Blocks

CRBN-targeting harnesses can be generally prepared according to Scheme D1:

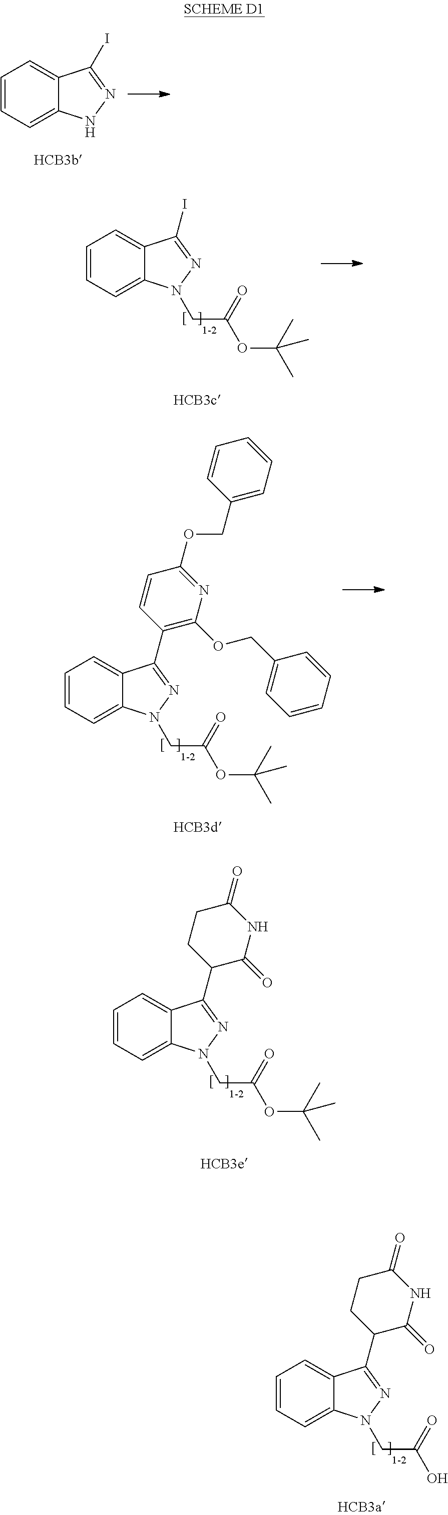

### Example 54. 2-(3-(2,6-dioxopiperidin-3-yl)-1H-indazol-1-yl)acetic acid (HCB49)

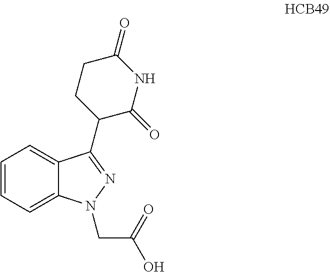 [SMILES: O=C(O)Cn1nc(C2CCC(=O)NC2=O)c2ccccc21]

Step 1: To a solution of 3-iodo-1H-indazole (300.00 mg, 1.23 mmol) in THF (8 mL) was added potassium tert-butoxide (0.21 g, 1.84 mmol) and the mixture was cooled to 0° C. The mixture was stirred at 0° C. for 30 min and then tert-butyl 2-bromoacetate (0.36 mL, 0.48 g, 2.46 mmol) was added. The reaction mixture was then stirred at room temperature overnight. The reaction mixture was diluted with EtOAc (50 mL), washed with water, and the aqueous layer was washed with EtOAc once. The combined organic layers were dried over sodium sulfate and concentrated. Silica gel column purification eluting with EtOAc:hexanes (0-70%) provided tert-butyl 2-(3-iodoindazol-1-yl)acetate (414 mg, 94%). LCMS: C₁₃H₁₅IN₂O₂ requires: 358, found: m/z=381 [M+Na]⁺.

Step 2: To a solution of 2,6-bis(benzyloxy)pyridin-3-ylboronic acid (50.00 mg, 0.15 mmol), tert-butyl 2-(3-iodoindazol-1-yl)acetate (53.43 mg, 0.15 mmol), and potassium carbonate (61.85 mg, 0.45 mmol) in dioxane (2 mL) and water (0.5 ml) was added tetrakis(triphenylphosphine)palladium(0) (34.48 mg, 0.03 mmol), and the reaction mixture was heated at 100° C. for one hour. The reaction was monitored by LCMS which indicated completion of the reaction. The reaction mixture was diluted with EtOAc (50 mL) and the organic layer was washed with brine, dried over sodium sulfate, filtered, and concentrated. Silica gel column purification eluting with EtOAc:hexane (5-100%) provided tert-butyl 2-{3-[2,6-bis(benzyloxy)pyridin-3-yl]indazol-1-yl}acetate (68 mg, 87%). LCMS: C₃₂H₃₁N₃O₄ requires: 521.2, found: m/z=522.6 [M+H]⁺.

Step 3: To tert-butyl 2-{3-[2,6-bis(benzyloxy)pyridin-3-yl]indazol-1-yl}acetate (510.00 mg, 0.98 mmol) and palladium on carbon (50 mg) in a 250 mL round-bottom flask was added THE (6 mL) and EtOH (9 mL) and the solution was purged and stirred under a hydrogen balloon for two hours. The reaction was monitored by LCMS which showed no starting material remained and desired product was the major product. The solids were filtered and the solvent was evaporated to afford tert-butyl 2-[3-(2,6-dioxopiperidin-3-yl)indazol-1-yl]acetate (310 mg, 92% yield). LCMS: C₁₈H₂₁N₃O₄ requires: 343.2, found: m/z=344.4 [M+H]⁺. ¹H NMR (500 MHz, Chloroform-d) δ 7.96 (s, 1H), 7.75 (d, J=8.2 Hz, 1H), 7.45 (t, J=7.7 Hz, 1H), 7.34 (d, J=8.5 Hz, 1H), 7.22 (t, J=7.6 Hz, 1H), 5.09-4.94 (m, 2H), 3.75 (qd, J=7.0, 1.5 Hz, 1H), 3.13-2.91 (m, 1H), 2.70 (dt, J=17.6, 6.1 Hz, 1H), 2.56 (dt, J=13.0, 6.6 Hz, 1H), 2.43 (dtd, J=14.4, 5.5, 2.7 Hz, 1H), 1.51-1.41 (m, 9H).

Step 4: Tert-butyl 2-[3-(2,6-dioxopiperidin-3-yl)indazol-1-yl]acetate (310.00 mg, 0.90 mmol) was dissolved in DCM (10 mL) and trifluoroacetic acid (5 mL) and the reaction stirred for two hours. The solution was concentrated under reduced pressure to afford 2-(3-(2,6-dioxopiperidin-3-yl)-1H-indazol-1-yl)acetic acid (HCB49) (259 mg, 100%). LCMS: C₁₄H₁₃N₃O₄ requires: 287.1, found: m/z=288.2 [M+H]⁺.

### Example 55. 3-(3-(2,6-dioxopiperidin-3-yl)-1H-indazol-1-yl)propanoic acid (HCB50)

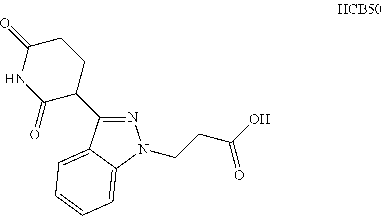 [SMILES: O=C(O)CCn1nc(C2CCC(=O)NC2=O)c2ccccc21]

3-(3-(2,6-dioxopiperidin-3-yl)-1H-indazol-1-yl)propanoic acid was synthesized with similar procedures immediately above. LCMS: C₁₅H₁₅N₃O₄ requires: 301.1, found: m/z=302.3 [M+H]. tert-butyl 2-[1-(5-bromopyridin-2-yl)piperidin-4-yl]acetate.

### Example 56. {1-[5-(2,6-dioxopiperidin-3-yl)pyridin-2-yl]piperidin-4-yl}acetic acid (HCB52)

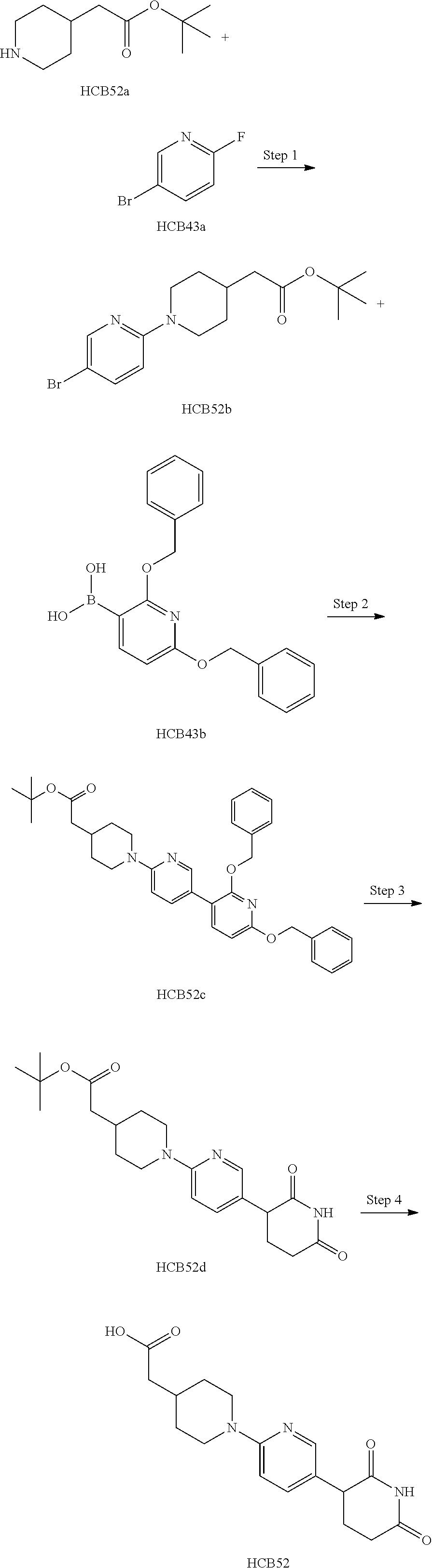

### Step 1: tert-butyl 2-[1-(5-bromopyridin-2-yl)piperidin-4-yl]acetate (HCB52b)

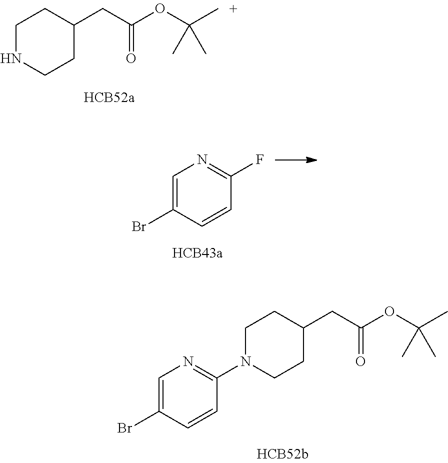

tert-butyl 2-(piperidin-4-yl)acetate (765 mg, 3.84 mmol), 5-bromo-2-fluoropyridine (0.40 mL, 676 mg, 3.84 mmol), and potassium carbonate (1.06 g, 7.68 mmol) were stirred in DMF (10.00 mL) overnight. Then the mixture was heated at 50° C. for six hours. The mixture was then stirred at room temperature for five days. The mixture was transferred to a separatory funnel with ethyl acetate and washed with two portions of water. The organic layer was dried over Na₂SO₄, filtered, and concentrated. The crude residue was purified by flash chromatography on a 40 g column (gradient elution with zero to 20% ethylacetate:hexanes) to provide tert-butyl 2-[1-(5-bromopyridin-2-yl)piperidin-4-yl]acetate (0.655 g, 48.0%). LCMS: C₁₆H₂₃BrN₂O₂ requires: 355, found: m/z=356 [M+H]⁺.

### Step 2: tert-butyl 2-{1-[2′,6′-bis(benzyloxy)-[3,3′-bipyridin]-6-yl]piperidin-4-yl}acetate (HCB52c)

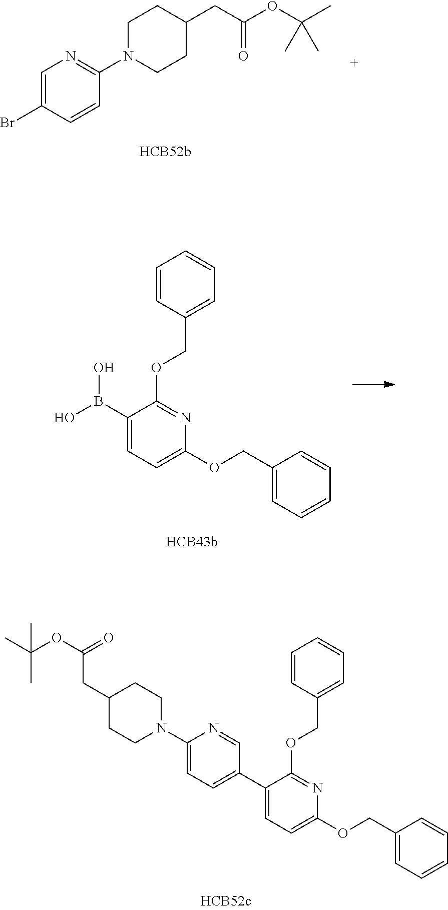

A mixture of 2,6-bis(benzyloxy)pyridin-3-ylboronic acid (495 mg, 1.48 mmol), tert-butyl 2-[1-(5-bromopyridin-2-yl)piperidin-4-yl]acetate (525 mg, 1.48 mmol), tetrakis(triphenylphosphine)palladium(0) (171 mg, 0.15 mmol), and potassium carbonate (408 mg, 2.96 mmol) in water (1.00 mL) and THE (3.00 mL) was microwaved at 120° C. for forty minutes. The water layer was removed by pipette. The organic layer was concentrated and then purified by flash chromatography on a 40 g column (gradient elution with zero to 35% ethyl acetate:hexanes) to provide tert-butyl 2-{1-[2′,6′-bis(benzyloxy)-[3,3′-bipyridin]-6-yl]piperidin-4-yl}acetate (0.303 g, 36.2%). LCMS: C₃₅H₃₉N₃O₄ requires: 565, found: m/z=566 [M+H]⁺.

### Step 3: tert-butyl 2-{1-[5-(2,6-dioxopiperidin-3-yl)pyridin-2-yl]piperidin-4-yl}acetate (HCB52d)

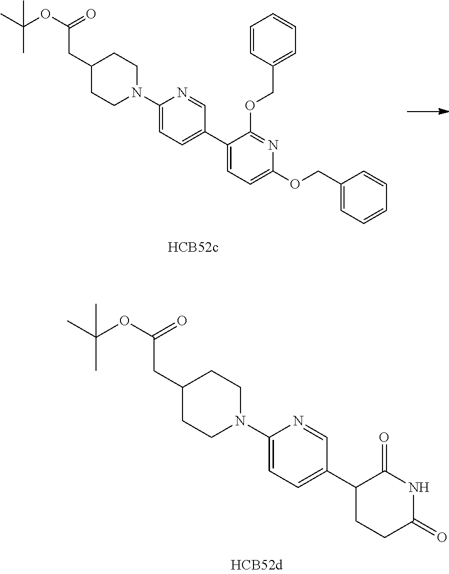 [SMILES: CC(C)(C)OC(=O)CC1CCN(c2ccc(-c3ccc(OCc4ccccc4)nc3OCc3ccccc3)cn2)CC1 ; CC(C)(C)OC(=O)CC1CCN(c2ccc(C3CCC(=O)NC3=O)cn2)CC1]

tert-butyl 2-{1-[2′,6′-bis(benzyloxy)-[3,3′-bipyridin]-6-yl]piperidin-4-yl}acetate (303 mg, 0.54 mmol) was suspended in EtOH (9.00 mL). THF (4 mL) was then added. 10% Pd/C (303 mg) was then added and the mixture was stirred under a balloon of H₂ for two hours. The mixture was diluted with THE (100 mL) and filtered through a pad of celite. The filtrate was concentrated. The crude residue was purified by flash chromatography on a 24 g column (gradient elution with zero to 10% MeOH:DCM) to provide tert-butyl 2-{1-[5-(2,6-dioxopiperidin-3-yl)pyridin-2-yl]piperidin-4-yl}acetate (0.152 g, 73.2%). LCMS: C₂₁H₂₉N₃O₄ requires: 387, found: m/z=388 [M+H]⁺.

### Step 4: {1-[5-(2,6-dioxopiperidin-3-yl)pyridin-2-yl]piperidin-4-yl}acetic acid (HCB52)

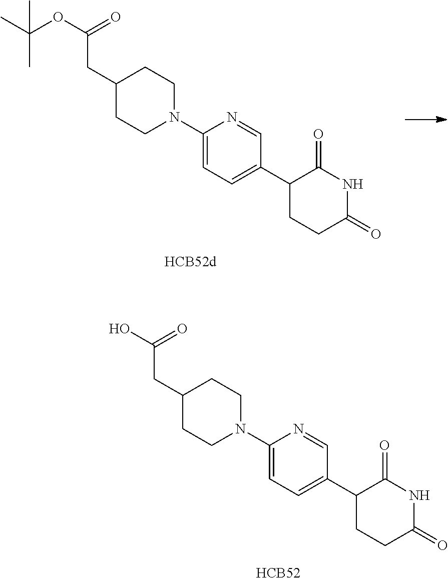 [SMILES: CC(C)(C)OC(=O)CC1CCN(c2ccc(C3CCC(=O)NC3=O)cn2)CC1 ; "HCB52" ; O=C(O)CC1CCN(c2ccc(C3CCC(=O)NC3=O)cn2)CC1]

tert-butyl 2-{1-[5-(2,6-dioxopiperidin-3-yl)pyridin-2-yl]piperidin-4-yl}acetate (29.00 mg, 0.07 mmol) was stirred in DCM (0.50 mL) and 4 M HCl solution in dioxane (0.50 mL, 0.07 g, 2.00 mmol). After stirring overnight, the mixture was concentrated in vacuo to provide {1-[5-(2,6-dioxopiperidin-3-yl)pyridin-2-yl]piperidin-4-yl}acetic acid (0.0248 g, 100%). LCMS: C₁₇H₂₁N₃O₄ requires: 331, found: m/z=332 [M+H]⁺.

### Example 57. 1-(5-(2,6-dioxopiperidin-3-yl)pyridin-2-yl)piperidine-4-carboxylic acid (HCB53)

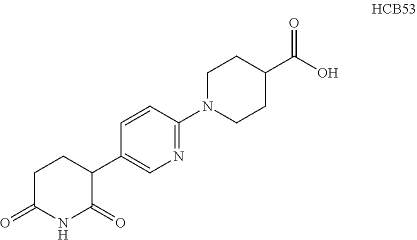 [SMILES: O=C1CCC(c2ccc(N3CCC(C(=O)O)CC3)nc2)C(=O)N1]

1-(5-(2,6-dioxopiperidin-3-yl)pyridin-2-yl)piperidine-4-carboxylic acid (HCB53) was prepared by procedures analogous to Example 54 starting from tert-butyl piperidine-4-carboxylate. LCMS: C₁₆H₁₉N₃O₄ requires: 317, found: m/z=318 [M+H]⁺.

### Example 58. 3-(4-(4-(((2R,6S)-2,6-dimethylpiperazin-1-yl)methyl)piperidin-1-yl)phenyl)piperidine-2,6-dione (HCB61)

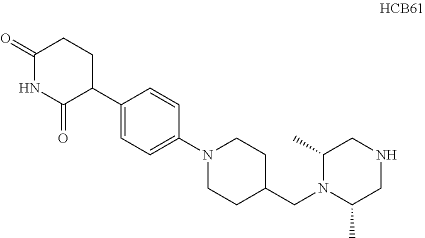 [SMILES: C[C@@H]1CNC[C@H](C)N1CC1CCN(c2ccc(C3CCC(=O)NC3=O)cc2)CC1]

### Step 1: Synthesis of tert-butyl (3R,5S)-4-((1-(4-(2,6-dioxopiperidin-3-yl)phenyl)piperidin-4-yl)methyl)-3,5-dimethylpiperazine-1-carboxylate (HCB61a)

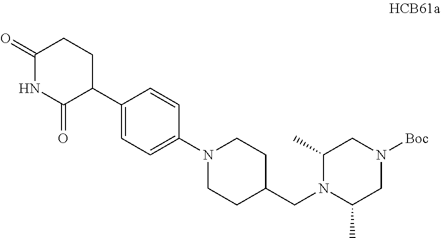 [SMILES: C[C@@H]1CN(C(=O)OC(C)(C)C)C[C@H](C)N1CC1CCN(c2ccc(C3CCC(=O)NC3=O)cc2)CC1]

A mixture of tert-butyl (3R,5S)-3,5-dimethylpiperazine-1-carboxylate (86 mg, 0.40 mmol), 1-(4-(2,6-dioxopiperidin-3-yl)phenyl)piperidine-4-carbaldehyd (100 mg, 0.40 mmol), i-Pr₂NEt (232 μL, 1.3 mmol) in DCM (3 mL) was allowed to stir at rt for 15 min. NaBH(OAc)₃ (212 mg, 1.0 mmol) was added, and the reaction mixture was allowed to stir at rt for 16 hr. The reaction was quenched with H₂O. The reaction mixture was extracted with 10% MeOH/DCM, and concentrated under reduced pressure. Flash chromatography (SiO₂, 0 to 5% MeOH/DCM gradient elution) provided tert-butyl (3R,5S)-4-((1-(4-(2,6-dioxopiperidin-3-yl)phenyl)piperidin-4-yl)methyl)-3,5-dimethylpiperazine-1-carboxylate (HCB 61a) (149 mg, 0.30 mmol, 90%). LCMS: C₂₈H₄₂N₄O₄ requires: 499, found: m/z=500 [M+H]⁺.

### Step 2: Synthesis of 3-(4-(4-(((2R,6S)-2,6-dimethylpiperazin-1-yl)methyl)piperidin-1-yl)phenyl)piperidine-2,6-dione (HCB61a)

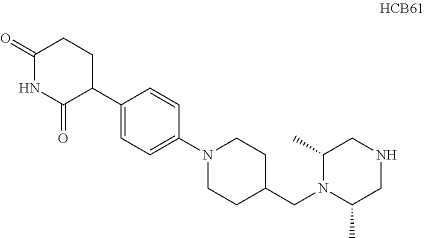 [SMILES: C[C@@H]1CNC[C@H](C)N1CC1CCN(c2ccc(C3CCC(=O)NC3=O)cc2)CC1]

Step 2: A mixture of tert-butyl (3R,5S)-4-((1-(4-(2,6-dioxopiperidin-3-yl)phenyl)piperidin-4-yl)methyl)-3,5-dimethylpiperazine-1-carboxylate (HCB 61a) (43 mg, 0.087 mmol) and HCl (4M in dioxane, 216 μL, 0.87 mmol) was stirred at rt for 16 hr. The resulting mixture was concentrated under a positive flow of N₂ to afford the title compound. LCMS: C₂₃H₃₄N₄O₂ requires: 398, found: m/z=399 [M+H]⁺.

### Example 59. 1-(4-(2,6-dioxopiperidin-3-yl)phenyl)piperidine-4-carbaldehyde (HCB62) (HCB62), (S)-1-(4-(2,6-dioxopiperidin-3-yl)phenyl)piperidine-4-carbaldehyde (HCB63), and (R)-1-(4-(2,6-dioxopiperidin-3-yl)phenyl)piperidine-4-carbaldehyde (HCB64)

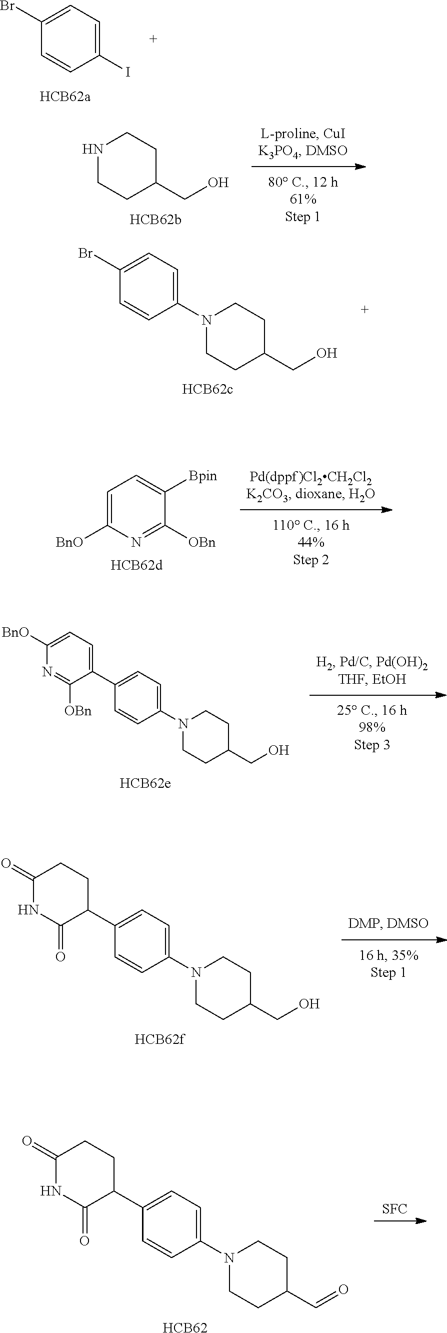

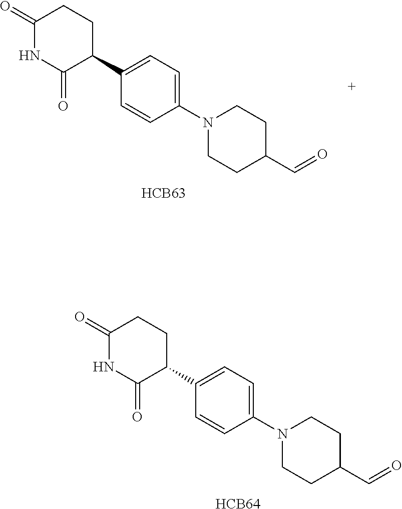

### Step 1: Synthesis of (1-(4-bromophenyl)piperidin-4-yl)methanol (HCB62c)

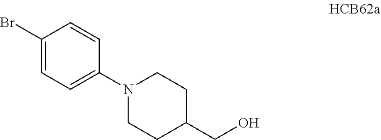 [SMILES: OCC1CCN(c2ccc(Br)cc2)CC1]

To a solution of piperidin-4-ylmethanol (264 g, 2.3 mol) in DMSO (2.5 L) was added 1-bromo-4-iodobenzene (500 g, 1.8 mol), K₃PO₄ (750 g, 3.5 mol), L-proline (46.3 g, 353 mmol) and CuI (67.3 g, 353 mmol) under N₂. The reaction was stirred at 80° C. for 12 hrs under N₂. LCMS showed that 1-bromo-4-iodobenzene was consumed and 63% of the desired mass was detected. The mixture was cooled to 20° C. and poured into water (3 L). The mixture was extracted with ethyl acetate (2.00 L×3) and washed with NH₃·H₂O in H₂O (500 ml NH₃·H₂O in 3.5 L H₂O) (1.50 L*2). The organic layers were washed with brine (2.00 L*2), dried over Na₂SO₄, filtered, concentrated, and purified by column chromatography (SiO₂, Petroleum ether/Ethyl acetate=20/1 to 2/1, R_{f}=0.44). (1-(4-bromophenyl)piperidin-4-yl)methanol (HCB62c) (1.2 kg, 3.2 mol, 61%, 72% purity) was obtained as light yellow solid. ¹H NMR (400 MHz, (CD₃)₂SO) δ 7.53-7.24 (m, 2H), 6.95-6.69 (m, 2H), 4.53-4.40 (m, 1H), 3.67 (d, J=12.4 Hz, 2H), 3.29-3.23 (m, 2H), 2.71-2.56 (m, 2H), 1.71 (d, J=13.2 Hz, 2H), 1.58-1.44 (m, 1H), 1.29-1.07 (m, 2H). LCMS: C₁₂H₁₆BrNO requires: 269, found: m/z=270 [M+H]⁺.

### Step 2: Synthesis of (1-(4-(2,6-bis(benzyloxy)pyridin-3-yl)phenyl)piperidin-4-yl)methanol (HCB62e)

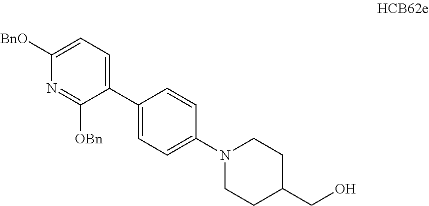 [SMILES: OCC1CCN(c2ccc(-c3ccc(OCc4ccccc4)nc3OCc3ccccc3)cc2)CC1]

To a solution of (1-(4-bromophenyl)piperidin-4-yl)methanol (100 g, 370 mmol), 2,6-bis(benzyloxy)-3-(4,4,5,5-tetramethyl-1,3,2-dioxaborolan-2-yl)pyridine (169 g, 407 mmol) in dioxane (1000 mL) and H₂O (200 mL) was added Pd(dppf)Cl₂ CH₂Cl₂ (30.2 g, 37 mmol) and K₂CO₃ (153 g, 1.1 mol) under N₂. Then the mixture was stirred at 110° C. for 16 hrs. LCMS showed (1-(4-bromophenyl)piperidin-4-yl)methanol was consumed and 41% desired mass was detected. The mixture was filtered. The filtrate was extracted with ethyl acetate (2000 mL*3). The combined organic layers were washed with brine (1000 mL), dried over Na₂SO₄, filtrated and concentrated under vacuum. The residue was purified by column chromatography (SiO₂, Petroleum ether/Ethyl acetate=3/1, R_{f}=0.1). The crude product was triturated with petroleum ether (1000 mL) at 25° C. for 30 mins. (1-(4-(2,6-bis(benzyloxy)pyridin-3-yl)phenyl)piperidin-4-yl)methanol (HCB62e) (325 g, 657 mmol, 44%, 97% purity) was obtained as white solid. ¹H NMR (400 MHz, (CD₃)₂SO) δ 7.62-7.72 (m, 1H), 7.23-7.48 (m, 12H), 6.86-6.99 (m, 2H), 6.43-6.55 (m, 1H), 5.30-5.43 (m, 4H), 4.44-4.52 (m, 1H), 3.65-3.77 (m, 2H), 3.24-3.32 (m, 2H), 2.59-2.69 (m, 2H), 1.68-1.78 (m, 2H), 1.46-1.57 (m, 1H), 1.17-1.29 (m, 2H). LCMS: C₃₁H₃₂N₂O₃ requires: 480, found: m/z=481 [M+H]⁺.

### Step 3: Synthesis of 3-(4-(4-(hydroxymethyl)piperidin-1-yl)phenyl)piperidine-2,6-dione (HCB62f)

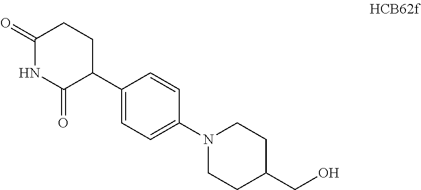 [SMILES: O=C1CCC(c2ccc(N3CCC(CO)CC3)cc2)C(=O)N1]

To a solution of (1-(4-(2,6-bis(benzyloxy)pyridin-3-yl)phenyl)piperidin-4-yl)methanol (70 g, 142 mmol, 98% purity) in THE (700 mL) and EtOH (700 mL) was added Pd/C (14.5 g, 10% purity) and Pd(OH)₂ (14.5 g, 20% purity) under N₂ atmosphere. The suspension was degassed and purged with H₂ for 3 times. The mixture was stirred under H₂ (15 psi) at 25° C. for 16 hr. LCMS showed the starting material was consumed and 90.4% desired mass was detected. The mixture was filtered through a pad of celite and the filtrate was concentrated under reduced pressure to give the crude product without further purification. 3-(4-(4-(hydroxymethyl)piperidin-1-yl)phenyl)piperidine-2,6-dione (HCB62f) (178 g, 556 mmol, 98%, 95% purity) was obtained as white solid. The title compound (325 g, 657 mmol, 44% yield, 97% purity) was obtained as white solid. ¹H NMR (400 MHz, (CD₃)₂SO) δ 10.71-10.81 (m, 1H), 6.97-7.09 (m, 2H), 6.81-6.92 (m, 2H), 4.41-4.50 (m, 1H), 4.28-4.38 (m, 1H), 3.66-3.75 (m, 2H), 3.37-3.49 (m, 2H), 3.24-3.31 (m, 2H), 2.56-2.68 (m, 3H), 1.96-2.20 (m, 2H), 1.67-1.77 (m, 2H), 1.42-1.57 (m, 1H), 1.15-1.28 (m, 2H). LCMS: C₁₇H₂₂N₂O₃ requires: 302, found: m/z=303 [M+H]⁺.

### Step 4: 1-(4-(2,6-dioxopiperidin-3-yl)phenyl)piperidine-4-carbaldehyde (HCB62)

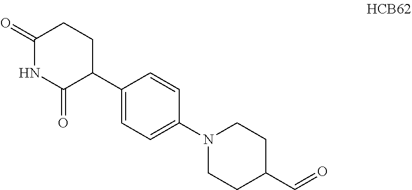 [SMILES: O=CC1CCN(c2ccc(C3CCC(=O)NC3=O)cc2)CC1]

To a solution of 3-(4-(4-(hydroxymethyl)piperidin-1-yl)phenyl)piperidine-2,6-dione (53.0 g, 165 mmol, 95% purity) in DMSO (530 mL) was slowly added DMP (140 g, 331 mmol, 102 mL) at 25° C. Then the mixture was stirred at 25° C. for 16 hrs. The mixture was adjusted with saturated aqueous Na₂CO₃ until pH=10 and the aqueous layer was extracted with ethyl acetate (3000 mL*3). The combined organic layers were washed with Na₂S₂O₃ solution (1000 mL) and brine (1000 mL), dried over Na₂SO₄, concentrated under vacuum. The crude product was triturated with ethyl acetate (500 mL) at 25° C. for 30 min. 1-(4-(2,6-dioxopiperidin-3-yl)phenyl)piperidine-4-carbaldehyde (HCB62) (54.0 g, 175 mmol, 35% yield, 97% purity) was obtained as yellow solid. ¹H NMR (400 MHz, (CD₃)₂SO) δ 10.77 (s, 1H), 9.53-9.70 (m, 1H), 6.99-7.12 (m, 2H), 6.79-6.96 (m, 2H), 3.67-3.77 (m, 1H), 3.48-3.63 (m, 2H), 2.72-2.88 (m, 2H), 2.57-2.68 (m, 1H), 2.38-2.49 (m, 2H), 1.88-2.20 (m, 4H), 1.49-1.66 (m, 2H).

### (S)-1-(4-(2,6-dioxopiperidin-3-yl)phenyl)piperidine-4-carbaldehyde (HCB63) (*Arbitrarily Assigned)

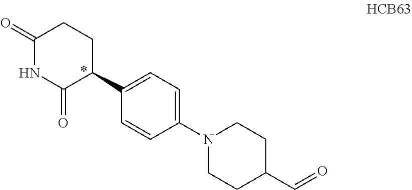 [SMILES: O=CC1CCN(c2ccc([C@@H]3CCC(=O)NC3=O)cc2)CC1]

The crude enantiomer was purified by prep-SFC (column: DAICEL CHIRALPAK AD (250 mm*30 mm, 10 μm); mobile phase: [IPA-ACN]; B %: 65%-65%, 4; 580 mins) to get the title compound (8.13 g, 27.07 mmol, 29%, 100% ee) as off-white solid.

### (R)-1-(4-(2,6-dioxopiperidin-3-yl)phenyl)piperidine-4-carbaldehyde (HCB64) (*Arbitrarily Assigned)

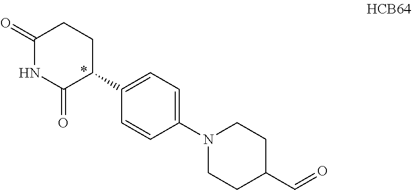 [SMILES: O=CC1CCN(c2ccc([C@H]3CCC(=O)NC3=O)cc2)CC1]

1-(4-(2,6-Dioxopiperidin-3-yl)phenyl)piperidine-4-carbaldehyde (HCB62) was purified by prep-SFC (column: DAICEL CHIRALPAK AD (250 mm×30 mm, 10 μm); mobile phase: [Neu-IPA]; B %: 65%-65%, 4.5; 650 min) to give (R)-1-(4-(2,6-dioxopiperidin-3-yl)phenyl)piperidine-4-carbaldehyde (HCB64) (*Arbitrarily Assigned) (13.64 g, 45.41 mmol, 49%, 98% ee) as yellow solid and crude enantiomer.

### Example 60. 1-(4-(2,6-dioxopiperidin-3-yl)-3-fluorophenyl)piperidine-4-carbaldehyde (HCB65)

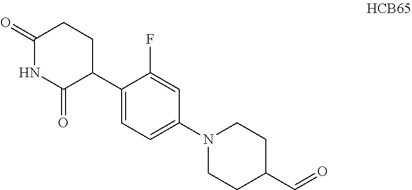 [SMILES: O=CC1CCN(c2ccc(C3CCC(=O)NC3=O)c(F)c2)CC1]

### Step 1: Synthesis of (1-(4-bromo-3-fluorophenyl)piperidin-4-yl)methanol (HCB65a)

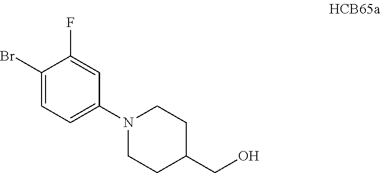 [SMILES: OCC1CCN(c2ccc(Br)c(F)c2)CC1]

Followed the procedure described in the procedure of (HCB62c) starting from 1-bromo-2-fluoro-4-iodobenzene (30 g, 99.7 mmol) to afford the title compound (11 g, 38.1 mmol, 38%). LCMS; C₁₂H₁₅BrFNO requires: 287, found: m/z=288 [M+H]⁺.

### Step 2: Synthesis of 3-(2-fluoro-4-(4-(hydroxymethyl)piperidin-1-yl)phenyl)piperidine-2,6-dione (HCB65b)

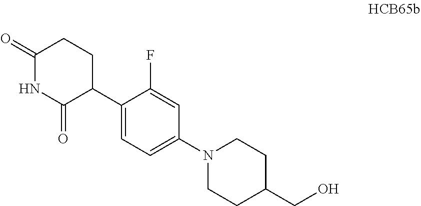 [SMILES: O=C1CCC(c2ccc(N3CCC(CO)CC3)cc2F)C(=O)N1]

Followed the procedure described in the procedure of (HCB62f) from (1-(4-(2,6-bis(benzyloxy)pyridin-3-yl)-3-fluorophenyl)piperidin-4-yl)methanol (17 g, 34.1 mmol) to afford the title compound (11.4 g, crude). LCMS: C₁₇H₂₁FN₂O₃ requires: 320, found: m/z=321 [M+H]⁺.

### Step 3: Synthesis of 1-(4-(2,6-dioxopiperidin-3-yl)-3-fluorophenyl)piperidine-4-carbaldehyde (HCB65)

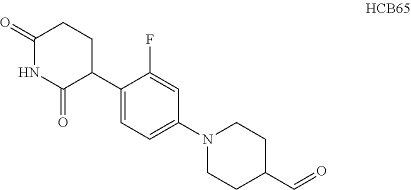 [SMILES: O=CC1CCN(c2ccc(C3CCC(=O)NC3=O)c(F)c2)CC1]

Followed the procedure described in the procedure of (HCB62) from 3-(2-fluoro-4-(4-(hydroxymethyl)piperidin-1-yl)phenyl)piperidine-2,6-dione (11.4 g, 35.5 mmol) to afford the title compound (2.06 g, 6.26 mmol, 18%). ¹H NMR (400 MHz, (CD₃)₂SO) δ 10.79 (s, 1H), 9.62 (s, 1H), 7.07 (t, J=8.8 Hz, 1H), 6.77-6.69 (m, 2H), 3.91-3.84 (m, 1H), 3.67-3.59 (m, 2H), 2.91-2.81 (m, 2H), 2.76-2.65 (m, 1H), 2.56-2.52 (m, 1H), 2.49-2.45 (m, 1H), 2.20-2.07 (m, 1H), 1.97-1.86 (m, 3H), 1.61-1.49 (m, 2H).

### Example 61. 1-(4-(2,6-dioxopiperidin-3-yl)-2-fluorophenyl)piperidine-4-carbaldehyde (HCB66)

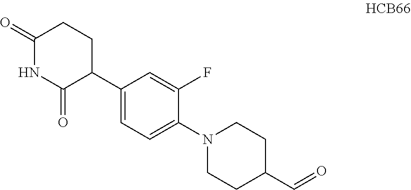 [SMILES: O=CC1CCN(c2ccc(C3CCC(=O)NC3=O)cc2F)CC1]

### Step 1: Synthesis of (1-(4-bromo-2-fluorophenyl)piperidin-4-yl)methanol (HCB66a)

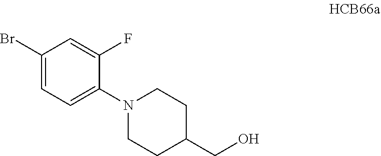 [SMILES: OCC1CCN(c2ccc(Br)cc2F)CC1]

Followed the procedure described in the procedure of (HCB62c) from 4-bromo-2-fluoro-1-iodobenzene (100 g, 332 mmol) to afford the title compound (10.7 g, 35.6 mmol, 11%). ¹H NMR (400 MHz, (CD₃)₂SO) δ 7.42-7.36 (m, 1H), 7.30-7.24 (m, 1H), 6.98 (t, J=9.2 Hz, 1H), 4.48 (t, J=5.2 Hz, 1H), 3.31-3.27 (m, 3H), 2.68-2.57 (m, 2H), 1.79-1.69 (m, 2H), 1.56-1.41 (m, 1H), 1.34-1.20 (m, 2H). LCMS; C₁₂H₁₅BrFNO requires: 287, found: m/z=288 [M+H]⁺.

### Step 2: Synthesis of 3-(3-fluoro-4-(4-(hydroxymethyl)piperidin-1-yl)phenyl)piperidine-2,6-dione (HCB66b)

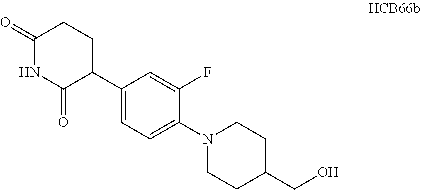 [SMILES: O=C1CCC(c2ccc(N3CCC(CO)CC3)c(F)c2)C(=O)N1]

Followed the procedure described in the procedure of (HCB62f) from (1-(4-(2,6-bis(benzyloxy)pyridin-3-yl)-2-fluorophenyl)piperidin-4-yl)methanol (11.6 g, 23.2 mmol) to afford the title compound 8.4 g, crude). LCMS: C₁₇H₂₁FN₂O₃ requires: 320, found: m/z=321 [M+H]⁺.

### Step 3: Synthesis of Synthesis of 1-(4-(2,6-dioxopiperidin-3-yl)-2-fluorophenyl)piperidine-4-carbaldehyde (HCB66)

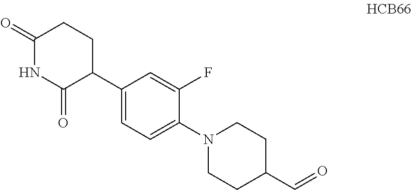 [SMILES: O=CC1CCN(c2ccc(C3CCC(=O)NC3=O)cc2F)CC1]

Followed the procedure described in the procedure of (HCB62) from 3-(3-fluoro-4-(4-(hydroxymethyl)piperidin-1-yl)phenyl)piperidine-2,6-dione (8.4 g, 26.2 mmol) to afford the title compound (2.5 g, 7.6 mmol, 29%). ¹H NMR (400 MHz, (CD₃)₂SO) δ 10.8 (s, 1H), 9.64 (s, 1H), 7.06-6.90 (s, 3H), 3.83-3.76 (m, 1H), 3.29-3.24 (m, 2H), 2.76 (t, J=10.0 Hz, 2H), 2.69-2.60 (m, 1H), 2.48-2.41 (m, 2H), 2.24-2.13 (m, 1H), 2.04-1.91 (m, 3H), 1.71-1.58 (m, 2H).

### Example 62. 3-(6-(4-(hydroxymethyl)piperidin-1-yl)-2-methylpyridin-3-yl)piperidine-2,6-dione (HCB67)

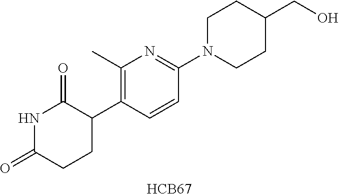 [SMILES: "HCB67" ; Cc1nc(N2CCC(CO)CC2)ccc1C1CCC(=O)NC1=O]

### Step 1: Synthesis of (1-(5-bromo-6-methylpyridin-2-yl)piperidin-4-yl)methanol

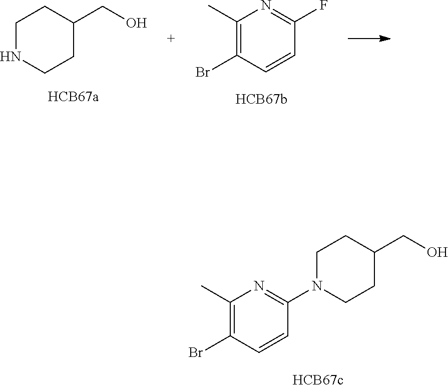

A mixture of 3-bromo-6-fluoro-2-methylpyridine (889 mg, 4.68 mmol), piperidin-4-ylmethanol (539 mg, 4.68 mmol), and potassium carbonate (1.29 g, 9.36 mmol) in DMF (10.00 mL) was heated at 70C for 4 hours. The mixture was partitioned between water and ethyl acetate. The organic layer was concentrated. The crude residue was purified by flash chromatography on a 40 g column eluted with 0 to 70% ethylacetate/hexanes to provide [1-(5-bromo-6-methylpyridin-2-yl)piperidin-4-yl]methanol (0.524 g, 39.3%). LCSM C₁₂H₁₇BrN₂O requires: 284, found: m/z=285 [M+H]⁺.

### Step 2: Synthesis of (1-(2′,6′-bis(benzyloxy)-2-methyl-[3,3′-bipyridin]-6-yl)piperidin-4-yl)methanol

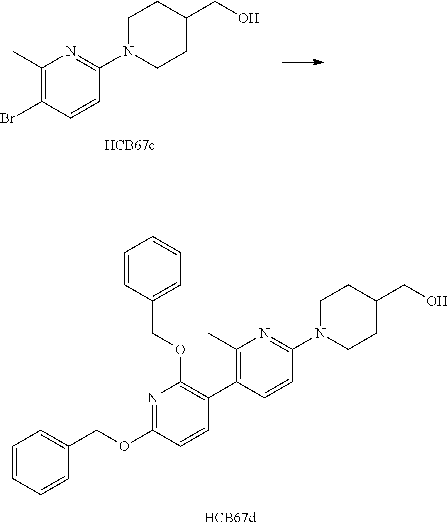 [SMILES: Cc1nc(N2CCC(CO)CC2)ccc1Br ; Cc1nc(N2CCC(CO)CC2)ccc1-c1ccc(OCc2ccccc2)nc1OCc1ccccc1]

A mixture of (1-(5-bromo-6-methylpyridin-2-yl)piperidin-4-yl)methanol (509 mg, 1.78 mmol), 2,6-bis(benzyloxy)pyridin-3-ylboronic acid (598 mg, 1.78 mmol), tetrakis(triphenylphosphine)palladium(0) (206 mg, 0.18 mmol), and potassium carbonate (493 mg, 3.57 mmol) in THE (3.00 mL) and water (1.00 mL) was microwaved at 120C for 1 hour. The water layer was removed with a pipette. The organic layer was loaded onto a silica loading cartridge and purified by flash chromatography on a 40 g column eluted with 0 to 70% ethylacetate/hexanes to provide (1-(2′,6′-bis(benzyloxy)-2-methyl-[3,3′-bipyridin]-6-yl)piperidin-4-yl)methanol (0.550 g, 62.2%). LCSM C₃₁H₃₃N₃O₃ requires: 495, found: m/z=496 [M+H]⁺.

### Step 3: Synthesis of 3-(6-(4-(hydroxymethyl)piperidin-1-yl)-2-methylpyridin-3-yl)piperidine-2,6-dione (HCB67)

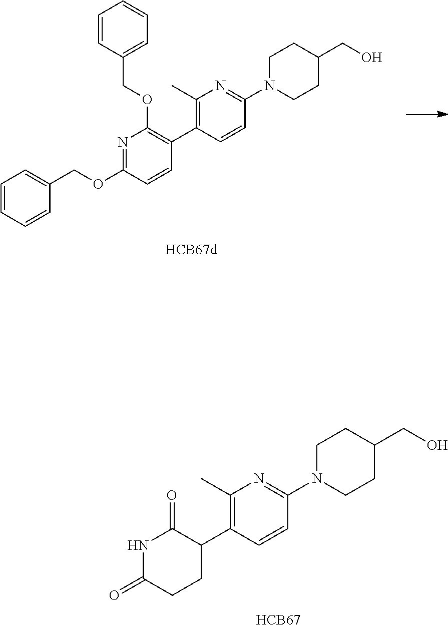 [SMILES: Cc1nc(N2CCC(CO)CC2)ccc1-c1ccc(OCc2ccccc2)nc1OCc1ccccc1 ; "HCB67" ; Cc1nc(N2CCC(CO)CC2)ccc1C1CCC(=O)NC1=O]

(1-(2′,6′-bis(benzyloxy)-2-methyl-[3,3′-bipyridin]-6-yl)piperidin-4-yl)methanol (550 mg, 1.11 mmol) and 10% Pd/C (550.00 mg) in EtOH (10.00 mL) and THE (10.00 mL) were stirred under a balloon of H₂ overnight. The mixture was diluted with 100 mL THE and filtered through a pad of celite. The mixture was concentrated. The crude residue was purified by flash chromatography on a 24 g column eluted with 0 to 10% MeOH/DCM to provide 3-(6-(4-(hydroxymethyl)piperidin-1-yl)-2-methylpyridin-3-yl)piperidine-2,6-dione (0.112 g, 31.8%). LCSM C₁₇H₂₃N₃O₃ requires: 317, found: m/z=318 [M+H]⁺.

### Example 63. 3-(4-(4-(hydroxymethyl)piperidin-1-yl)-2-methylphenyl)piperidine-2,6-dione (HCB68)

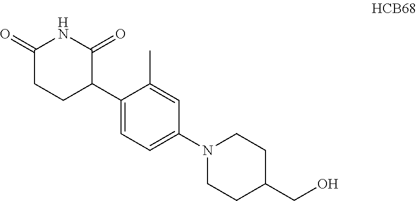 [SMILES: Cc1cc(N2CCC(CO)CC2)ccc1C1CCC(=O)NC1=O]

### Step 1: Synthesis of (1-(4-bromo-3-methylphenyl)piperidin-4-yl)methanol

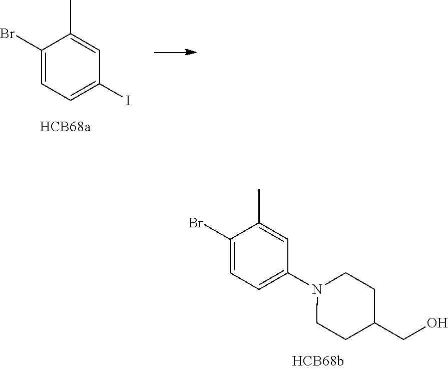

To a mixture of 1-bromo-4-iodo-2-methylbenzene (1.00 g, 3.37 mmol) in DMSO (5.00 mL) was added piperidin-4-ylmethanol (0.50 g, 4.38 mmol), L-proline (78 mg, 0.67 mmol), copper(I) iodide (128 mg, 0.67 mmol), and tripotassium phosphate (1.43 g, 6.74 mmol). The mixture was heated in an 85° C. heating block for 4 hours. The mixture was poured into water and extracted twice with ethyl acetate. The combined organic layers were dried over Na₂SO₄ and concentrated. The crude residue was purified by flash chromatography on a 40 g column eluted with 0 to 100% ethylacetate/hexanes to provide (1-(4-bromo-3-methylphenyl)piperidin-4-yl)methanol (0.445 g, 46.5%). LCSM C₁₃H₁₈BrNO requires: 283, found: m/z=284 [M+H]⁺.

### Step 2: Synthesis of (1-(4-(2,6-bis(benzyloxy)pyridin-3-yl)-3-methylphenyl)piperidin-4-yl)methanol

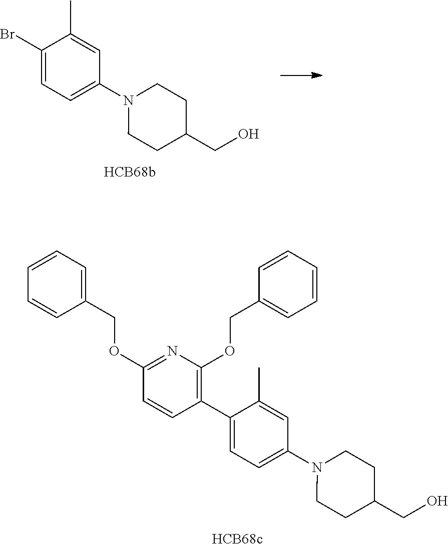 [SMILES: Cc1cc(N2CCC(CO)CC2)ccc1Br ; Cc1cc(N2CCC(CO)CC2)ccc1-c1ccc(OCc2ccccc2)nc1OCc1ccccc1]

(1-(4-bromo-3-methylphenyl)piperidin-4-yl)methanol (445 mg, 1.57 mmol), 2,6-bis(benzyloxy)pyridin-3-ylboronic acid (525 mg, 1.57 mmol), potassium carbonate (433 mg, 3.13 mmol) and tetrakis(triphenylphosphine)palladium(0) (181 mg, 0.16 mmol) in THE (3.00 mL) and water (1.00 mL) was microwaved at 120° C. for 1 hour. The aq. layer was removed with a pipette. The organic layer was loaded onto a silica loading cartridge. The mixture was purified by flash chromatography on a 40 g column eluted with 0 to 75% ethylacetate/hexanes to provide (1-(4-(2,6-bis(benzyloxy)pyridin-3-yl)-3-methylphenyl)piperidin-4-yl)methanol (0.538 g, 69.5%). LCSM C₃₂H₃₄N₂O₃ requires: 494, found: m/z=495 [M+H]⁺.

### Step 3: Synthesis of 3-(4-(4-(hydroxymethyl)piperidin-1-yl)-2-methylphenyl)piperidine-2,6-dione (HCB68)

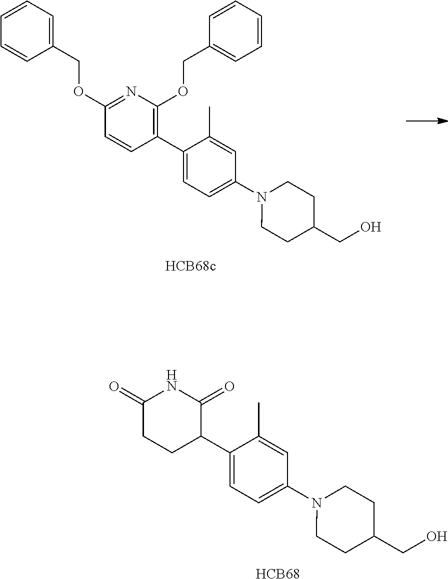 [SMILES: Cc1cc(N2CCC(CO)CC2)ccc1-c1ccc(OCc2ccccc2)nc1OCc1ccccc1 ; "HCB68" ; Cc1cc(N2CCC(CO)CC2)ccc1C1CCC(=O)NC1=O]

(1-(4-(2,6-bis(benzyloxy)pyridin-3-yl)-3-methylphenyl)piperidin-4-yl)methanol (538 mg, 1.09 mmol) and 10% Pd/C (538 mg) were stirred in THE (10.00 mL) and EtOH (10.00 mL) under a balloon of H₂ for 24 hours. The mixture was diluted with THE and filtered through celite. The resulting mixture was concentrated in vacuo. The crude residue was purified by flash chromatography on a 24 g column eluted with 0 to 20% MeOH/DCM to provide 3-(4-(4-(hydroxymethyl)piperidin-1-yl)-2-methylphenyl)piperidine-2,6-dione (0.129 g, 37.5%). LCSM C₁₈H₂₄N₂O₃ requires: 316, found: m/z=317 [M+H]⁺.

### Example 64. 3-(5-fluoro-6-(4-(hydroxymethyl)piperidin-1-yl)pyridin-3-yl)piperidine-2,6-dione (HCB69)

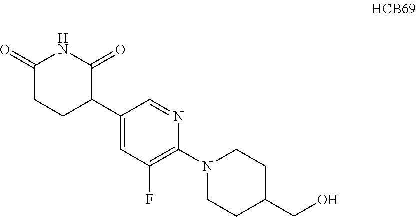 [SMILES: O=C1CCC(c2cnc(N3CCC(CO)CC3)c(F)c2)C(=O)N1]

### Step 1: Synthesis of (1-(5-bromo-3-fluoropyridin-2-yl)piperidin-4-yl)methanol

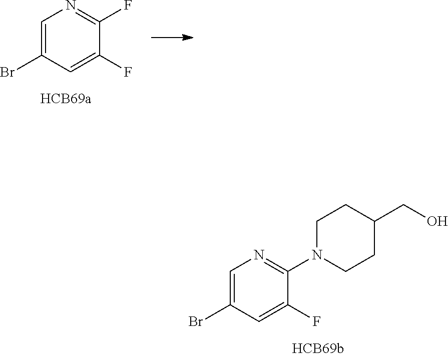 [SMILES: Fc1cc(Br)cnc1F ; OCC1CCN(c2ncc(Br)cc2F)CC1]

5-bromo-2,3-difluoropyridine (993 mg, 5.12 mmol), piperidin-4-ylmethanol (590 mg, 5.12 mmol), and potassium carbonate (1.41 g, 10.24 mmol) were stirred in DMF (10.00 mL) for 1 hour. The mixture was poured into water then extracted with ethyl acetate. The organic layer was dried over Na₂SO₄ and concentrated. The crude residue was purified by flash chromatography on a 40 g column eluted with 0 to 50% ethyl acetate/hexanes to provide (1-(5-bromo-3-fluoropyridin-2-yl)piperidin-4-yl)methanol (0.983 g, 66.4%). LCSM C₁₁H₁₄BrFN₂O requires: 288, found: m/z=289 [M+H]⁺.

### Step 2: Synthesis of (1-(2′,6′-bis(benzyloxy)-5-fluoro-[3,3′-bipyridin]-6-yl)piperidin-4-yl)methanol

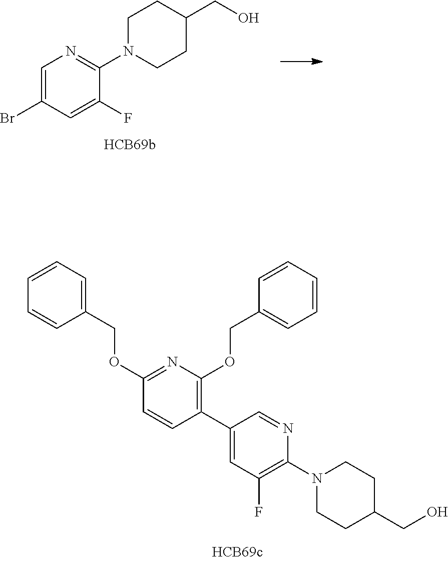 [SMILES: OCC1CCN(c2ncc(Br)cc2F)CC1 ; OCC1CCN(c2ncc(-c3ccc(OCc4ccccc4)nc3OCc3ccccc3)cc2F)CC1]

(1-(5-bromo-3-fluoropyridin-2-yl)piperidin-4-yl)methanol (507 mg, 1.75 mmol), 2,6-bis(benzyloxy)pyridin-3-ylboronic acid (588 mg, 1.75 mmol), potassium carbonate (485 mg, 3.51 mmol), and tetrakis(triphenylphosphine)palladium(0) (203 mg, 0.18 mmol) were deposited in a microwave vial followed by THE (3.00 mL) and water (1.00 mL). The headspace was flushed with N₂ and the vial was capped then microwaved at 120° C. for 1 hour. The aq. layer was removed. The organic layer was loaded onto a silica loading cartridge and purified by flash chromatography on a 40 g column eluted with 0 to 75% ethyl acetate/hexanes to provide (1-(2′,6′-bis(benzyloxy)-5-fluoro-[3,3′-bipyridin]-6-yl)piperidin-4-yl)methanol (0.636 g, 72.6%). LCSM C₃₀H₃₀FN₃O₃ requires: 499, found: m/z=500 [M+H]⁺.

### Step 3: Synthesis of 3-(5-fluoro-6-(4-(hydroxymethyl)piperidin-1-yl)pyridin-3-yl)piperidine-2,6-dione (HCB69)

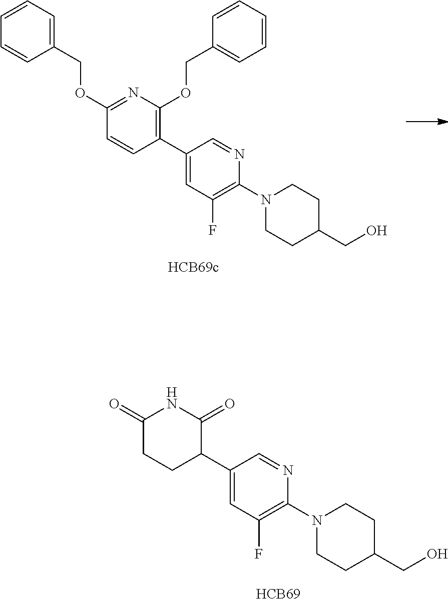 [SMILES: OCC1CCN(c2ncc(-c3ccc(OCc4ccccc4)nc3OCc3ccccc3)cc2F)CC1 ; "HCB69" ; O=C1CCC(c2cnc(N3CCC(CO)CC3)c(F)c2)C(=O)N1]

To a mixture of (1-(2′,6′-bis(benzyloxy)-5-fluoro-[3,3′-bipyridin]-6-yl)piperidin-4-yl)methanol (829 mg, 1.66 mmol) in THE (15.00 mL) and EtOH (15.00 mL) was added 10% Pd/C (829 mg). The mixture stirred under a balloon of H₂ for 3.5 hours. 100 mL THE was added and the mixture was filtered through a pad of celite. The resulting solution was concentrated. The crude residue was purified by flash chromatography on a 40 g column eluted with 0 to 10% MeOH/DCM to provide 3-(5-fluoro-6-(4-(hydroxymethyl)piperidin-1-yl)pyridin-3-yl)piperidine-2,6-dione (0.306 g, 57.4%). LCSM C₁₆H₂₀FN₃O₃ requires: 321, found: m/z=322 [M+H]⁺.

### Example 65. 3-(4-(4-(hydroxymethyl)piperidin-1-yl)-2-methoxyphenyl)piperidine-2,6-dione (HCB70)

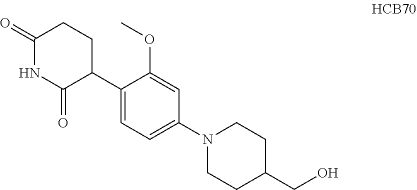 [SMILES: COc1cc(N2CCC(CO)CC2)ccc1C1CCC(=O)NC1=O]

### Step 1: Synthesis of (1-(4-bromo-3-methoxyphenyl)piperidin-4-yl)methanol

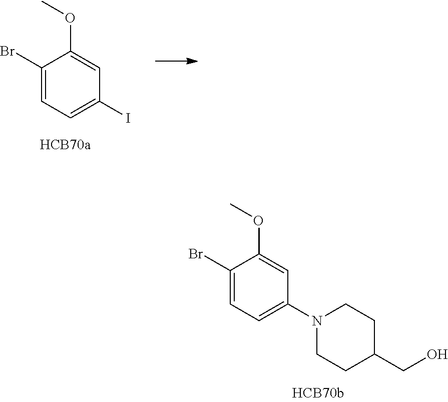

1-bromo-4-iodo-2-methoxybenzene (1.13 g, 3.61 mmol), piperidin-4-ylmethanol (540 mg, 4.69 mmol), copper(I) iodide (137 mg, 0.72 mmol), L-proline (83 mg, 0.72 mmol), and tripotassium phosphate (1.53 g, 7.22 mmol) in DMSO (5.00 mL) were deposited into a vial which was flushed with N₂ then heated at 85C overnight. After sitting for 2 days, water and ethyl acetate were added. The organic layer was dried over Na₂SO₄ and concentrated. The crude residue was purified by flash chromatography on a 40 g column eluted with 0 to 75% ethylacetate/hexanes to provide (1-(4-bromo-3-methoxyphenyl)piperidin-4-yl)methanol (0.763 g, 70.4%). LCSM C₁₃H₁₈BrNO₂ requires: 299, found: m/z=300 [M+H]⁺.

### Step 2: Synthesis of (1-(4-(2,6-bis(benzyloxy)pyridin-3-yl)-3-methoxyphenyl)piperidin-4-yl)methanol

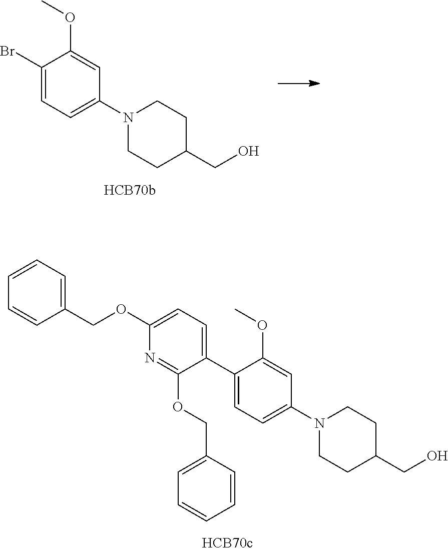 [SMILES: COc1cc(N2CCC(CO)CC2)ccc1Br ; COc1cc(N2CCC(CO)CC2)ccc1-c1ccc(OCc2ccccc2)nc1OCc1ccccc1]

(1-(4-bromo-3-methoxyphenyl)piperidin-4-yl)methanol (760 mg, 2.53 mmol), 2,6-bis(benzyloxy)pyridin-3-ylboronic acid (849 mg, 2.53 mmol), tetrakis(triphenylphosphine)palladium(0) (293 mg, 0.25 mmol), and potassium carbonate (700 mg, 5.06 mmol) were loaded in a microwave vial with THF (3.00 mL) and water (1.00 mL). The mixture was microwaved at 120C for 1 hour. The aq. layer was removed with a pipette. The organic layer was loaded onto a silica cartridge and purified by flash chromatography on a 40 g column eluted with 0 to 50% Ethylacetate/DCM to provide (1-(4-(2,6-bis(benzyloxy)pyridin-3-yl)-3-methoxyphenyl)piperidin-4-yl)methanol (0.363 g, 28.1%). LCSM C₃₂H₃₄N₂O₄ requires: 510, found: m/z=511 [M+H]⁺.

### Step 3: Synthesis of 3-(4-(4-(hydroxymethyl)piperidin-1-yl)-2-methoxyphenyl)piperidine-2,6-dione (HCB70)

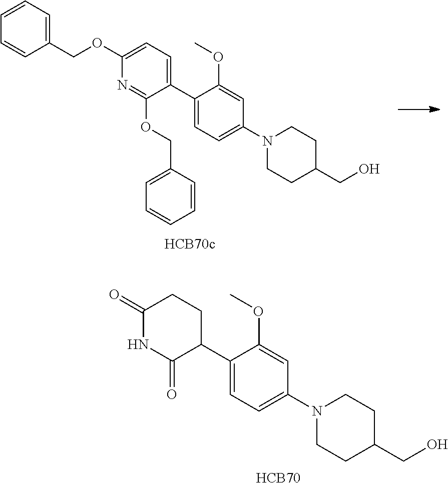 [SMILES: COc1cc(N2CCC(CO)CC2)ccc1-c1ccc(OCc2ccccc2)nc1OCc1ccccc1 ; "HCB70" ; COc1cc(N2CCC(CO)CC2)ccc1C1CCC(=O)NC1=O]

To a mixture of (1-(4-(2,6-bis(benzyloxy)pyridin-3-yl)-3-methoxyphenyl)piperidin-4-yl)methanol (363 mg, 0.71 mmol) in THF (10.00 mL) and EtOH (10.00 mL) was added 10% Pd/C (363 mg). The mixture was stirred under a balloon of H₂ for 4 hours. The mixture was diluted with 100 mL THE and filtered through a pad of celite. The resulting solution was concentrated to provide 3-(4-(4-(hydroxymethyl)piperidin-1-yl)-2-methoxyphenyl)piperidine-2,6-dione (0.233 g, 98.6%). LCSM C₁₈H₂₄N₂O₄ requires: 332, found: m/z=333 [M+H]⁺.

### Example 66. 3-(2-(4-(hydroxymethyl)piperidin-1-yl)pyridin-4-yl)piperidine-2,6-dione (HCB71)

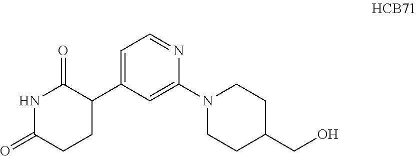 [SMILES: O=C1CCC(c2ccnc(N3CCC(CO)CC3)c2)C(=O)N1]

### Step 1: Synthesis of (1-(4-bromopyridin-2-yl)piperidin-4-yl)methanol

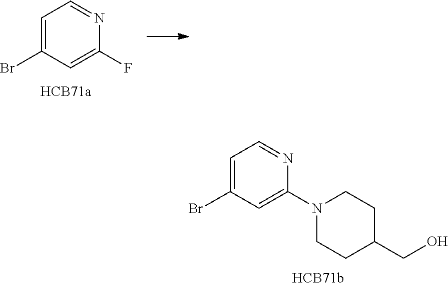 [SMILES: Fc1cc(Br)ccn1 ; OCC1CCN(c2cc(Br)ccn2)CC1]

4-Bromo-2-fluoropyridine (1.02 g, 5.78 mmol), potassium carbonate (2.40 g, 17.3 mmol), and piperidin-4-ylmethanol (799 mg, 6.93 mmol) in DMSO (14.00 mL) was heated at 100° C. for 1 hour. The mixture was poured into water then extracted with ethyl acetate. The organic layer was dried over Na₂SO₄ and concentrated in vacuo. The crude residue was purified by flash chromatography on a 40 g column eluted with 0 to 50% ethylacetate/DCM to provide (1-(4-bromopyridin-2-yl)piperidin-4-yl)methanol (1.32 g, 84.2%). LCSM C_{II}H₁₅BrN₂O requires: 270, found: m/z=271 [M+H]⁺.

### Step 2: Synthesis of (1-(2,6-bis(benzyloxy)-[3,4′-bipyridin]-2′-yl)piperidin-4-yl)methanol

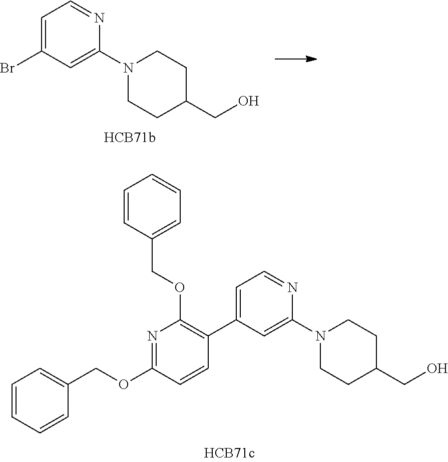 [SMILES: OCC1CCN(c2cc(Br)ccn2)CC1 ; OCC1CCN(c2cc(-c3ccc(OCc4ccccc4)nc3OCc3ccccc3)ccn2)CC1]

A mixture of (1-(4-bromopyridin-2-yl)piperidin-4-yl)methanol (402 mg, 1.48 mmol), 2,6-bis(benzyloxy)pyridin-3-ylboronic acid (497 mg, 1.48 mmol), tetrakis(triphenylphosphine)palladium(0) (171 mg, 0.15 mmol) and potassium carbonate (410 mg, 2.97 mmol) in THE (3.00 mL) and Water (1.00 mL) was microwaved at 120° C. for 40 minutes. The water layer was removed and the organic layer was concentrated. The organic layer was purified by flash chromatography on a 40 g column eluted with 0 to 100% ethyl acetate/DCM to provide (1-(2,6-bis(benzyloxy)-[3,4′-bipyridin]-2′-yl)piperidin-4-yl)methanol (0.315 g, 44.1%). LCSM C₃₀H₃₁N₃O₃ requires: 481, found: m/z=482 [M+H]⁺.

### Step 3: Synthesis of 3-(2-(4-(hydroxymethyl)piperidin-1-yl)pyridin-4-yl)piperidine-2,6-dione

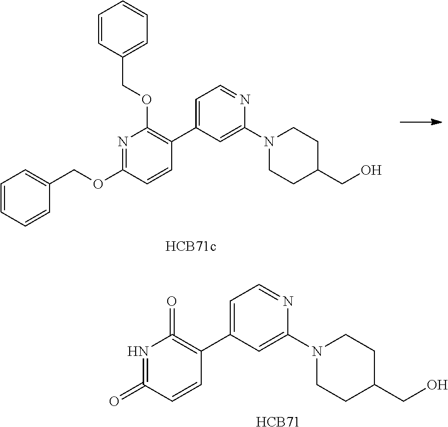

(1-(2,6-bis(benzyloxy)-[3,4′-bipyridin]-2′-yl)piperidin-4-yl)methanol (315 mg, 0.65 mmol) and 10% Pd/C (315 mg) were stirred in EtOH (6.00 mL) and THE (3.00 mL) under a balloon of H₂ for 4 hours. The mixture was diluted with 100 mL THE and filtered through celite. The celite pad was washed with MeOH and DCM. The resulting solution was concentrated. The crude residue purified by flash chromatography on a 24 g column eluted with 0 to 10% MeOH/DCM to provide 3-(2-(4-(hydroxymethyl)piperidin-1-yl)pyridin-4-yl)piperidine-2,6-dione (0.0729 g, 36.7%). LCSM C₁₆H₂₁N₃O₃ requires: 303, found: m/z=304 [M+H]⁺.

### Example 67. 3-(2-(4-(hydroxymethyl)piperidin-1-yl)pyrimidin-5-yl)piperidine-2,6-dione (HCB72)

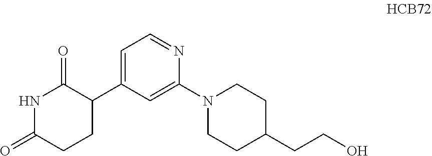 [SMILES: O=C1CCC(c2ccnc(N3CCC(CCO)CC3)c2)C(=O)N1]

### Step 1: Synthesis of 2-(1-(4-bromopyridin-2-yl)piperidin-4-yl)ethan-1-ol

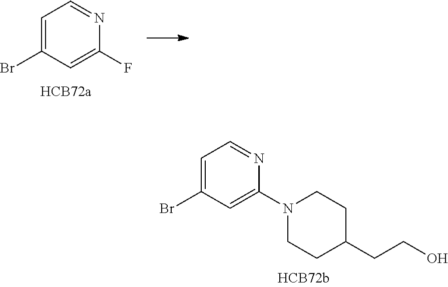 [SMILES: Fc1cc(Br)ccn1 ; OCCC1CCN(c2cc(Br)ccn2)CC1]

A mixture of 4-bromo-2-fluoropyridine (1.05 g, 5.97 mmol), 4-piperidineethanol (925 mg, 7.16 mmol), and potassium carbonate (2.47 g, 17.9 mmol) in DMSO (14.00 mL) was heated at 100° C. for 45 minutes. The mixture was cooled and transferred to a separatory funnel with ethyl acetate and water. The organic layer was dried over Na₂SO₄ and concentrated. The crude residue was purified by flash chromatography on an 80 g column eluted with 0 to 100% ethylacetate/DCM to provide2-(1-(4-bromopyridin-2-yl)piperidin-4-yl)ethan-1-ol (1.44 g, 84.3%). LCSM C₁₂H₁₇BrN₂O requires: 284, found: m/z=285 [M+H]⁺.

### Step 2: Synthesis of 2-(1-(2,6-bis(benzyloxy)-[3,4′-bipyridin]-2′-yl)piperidin-4-yl)ethan-1-ol

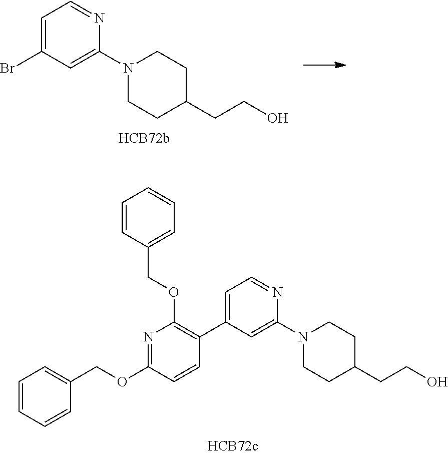 [SMILES: OCCC1CCN(c2cc(Br)ccn2)CC1 ; OCCC1CCN(c2cc(-c3ccc(OCc4ccccc4)nc3OCc3ccccc3)ccn2)CC1]

A mixture of 2-(1-(4-bromopyridin-2-yl)piperidin-4-yl)ethan-1-ol (440.00 mg, 1.54 mmol), 2,6-bis(benzyloxy)pyridin-3-ylboronic acid (517 mg, 1.54 mmol), tetrakis(triphenylphosphine)palladium(0) (178 mg, 0.15 mmol), and potassium carbonate (426 mg, 3.09 mmol) in THE (3.00 mL) and Water (1.00 mL) was microwaved at 120° C. for 40 minutes. The aqueous layer was removed with a pipette. The organic layer was loaded directly onto a silica gel loading cartridge. The material was purified by flash chromatography on a 40 g column eluted with 0 to 100% ethylacetate/DCM to provide 2-(1-(2,6-bis(benzyloxy)-[3,4′-bipyridin]-2′-yl)piperidin-4-yl)ethan-1-ol (0.4750 g, 62.1%). LCSM C₃₁H₃₃N₃O₃ requires: 495, found: m/z=496 [M+H]⁺.

### Step 3: Synthesis of 3-(2-(4-(2-hydroxyethyl)piperidin-1-yl)pyridin-4-yl)piperidine-2,6-dione (HCB72)

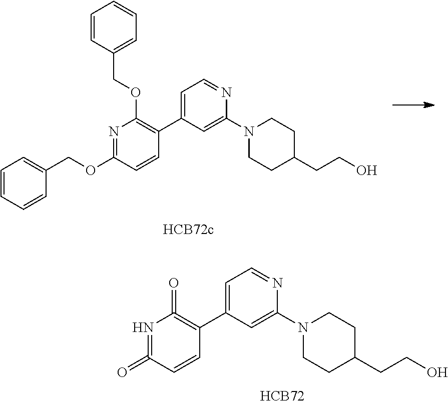

To a mixture of 2-(1-(2,6-bis(benzyloxy)-[3,4′-bipyridin]-2′-yl)piperidin-4-yl)ethan-1-ol (475 mg, 0.96 mmol) in EtOH (10.00 mL) and THE (5.00 mL) was added 10% Pd/C (475 mg). The mixture was stirred under a balloon of H₂ for 4 hours. The mixture was diluted with 50 mL THE and was filtered through celite. The pad of celite was washed with additional THE and DCM. The resulting solution was concentrated. The crude residue was purified by flash chromatography on a 40 g column eluted with 0 to 10% MeOH/DCM to provide 3-(2-(4-(2-hydroxyethyl)piperidin-1-yl)pyridin-4-yl)piperidine-2,6-dione (0.0213 g, 7.0%). LCSM C₁₇H₂₃N₃O₃ requires: 317, found: m/z=318 [M+H]⁺.

### Example 68. 3-(6-((S)-3-methyl-4-(piperidin-4-ylmethyl)piperazin-1-yl)pyridin-3-yl)piperidine-2,6-dione (HCB73)

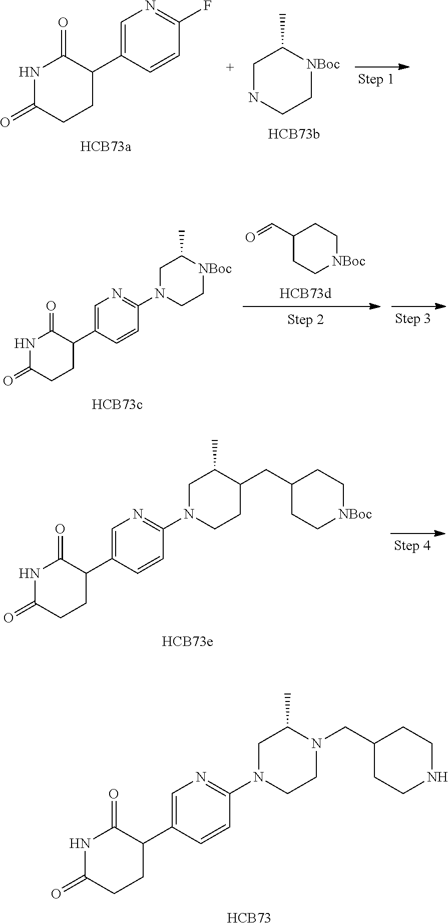

### Step 1: Synthesis of tert-butyl (2S)-4-(5-(2,6-dioxopiperidin-3-yl)pyridin-2-yl)-2-methylpiperazine-1-carboxylate

To a 20 mL microwave reaction vial was added rac-(3R)-3-(6-fluoropyridin-3-yl)piperidine-2,6-dione (500.00 mg, 2.40 mmol), tert-butyl (2S)-2-methylpiperazine-1-carboxylate (577.20 mg, 2.88 mmol), N,N-diisopropylethylamine (1.68 mL, 1.24 g, 9.61 mmol), and DMSO (5.00 mL). The reaction mixture was stirred at 180 C for 5 h by MW. The reaction mixture was concentrated under reduced pressure, and the crude product carried forward to next reaction without further purification. LCMS: C₂₀H₂₈N₄O₄ requires: 388.2, found: m/z=389.2 [M+H]⁺.

### Step 2: Synthesis of 3-(6-((S)-3-methylpiperazin-1-yl)pyridin-3-yl)piperidine-2,6-dione

To a 100 mL round bottom flask was added tert-butyl (2S)-4-{5-[(3RS)-2,6-dioxopiperidin-3-yl]pyridin-2-yl}-2-methylpiperazine-1-carboxylate (932.00 mg, 2.40 mmol) and DCM (3 mL). To the reaction mixture was added trifluoroacetic acid (2.77 mL, 4.10 g, 35.99 mmol). After 30 min, reaction mixture was concentrated under reduced pressure. The resulting residue was purified by reverse phase column chromatography (415 g C18 silica, 0-20% MeCN+0.1% TFA/H₂O+0.1% TFA) to yield the title compound as a white solid (151 mg, 22%). LCMS: C₁₅H₂₀N₄O₂ requires: 288.2, found: m/z=289.5 [M+H]⁺.

### Step 3: Synthesis of tert-butyl 4-(((2S)-4-(5-(2,6-dioxopiperidin-3-yl)pyridin-2-yl)-2-methylpiperazin-1-yl)methyl)piperidine-1-carboxylate

To a 2 dram vial was added (3R,S)-3-{6-[(3S)-3-methylpiperazin-1-yl]pyridin-3-yl}piperidine-2,6-dione (20.00 mg, 0.07 mmol), tert-butyl 4-formylpiperidine-1-carboxylate (16.27 mg, 0.08 mmol), DMSO (0.10 mL), and DCM (1.00 mL). To the reaction mixture was added sodium triacetoxyborohydride (0.04 g, 0.21 mmol) in one portion. After 1 h, the reaction mixture was quenched with sat. NaHCO₃ (aq), and the product was extracted 3× with DCM. The resulting residue was purified by column chromatography (4 g silica, 50-100% EtOAc/DCM) to yield the title compound as a white solid (29 mg, 86%). LCMS: C₂₆H₃₉N₅O₄ requires: 485.3, found: m/z=486.4 [M+H]⁺.

### Step 4: Synthesis of 3-(6-((S)-3-methyl-4-(piperidin-4-ylmethyl)piperazin-1-yl)pyridin-3-yl)piperidine-2,6-dione (HCB73)

To a 20 mL vial was added tert-butyl 4-{[(2S)-4-{5-[(3RS)-2,6-dioxopiperidin-3-yl]pyridin-2-yl}-2-methylpiperazin-1-yl]methyl}piperidine-1-carboxylate (29.00 mg, 0.06 mmol) and DCM (0.30 mL). To the mixture was added 4N hydrogen chloride in dioxane (1.34 mL, 0.20 g, 5.37 mmol) in a dropwise fashion. After 1 h, the reaction mixture was concentrated under reduced pressure to yield the title compound as a white solid (44 mg, 100%). LCMS: C₂₁H₃₁N₅O₂ requires: 385.2, found: m/z=386.3 [M+H]⁺.

### Example 69. 3-(6-(4-(piperidin-4-ylmethyl)-1,4-diazepan-1-yl)pyridin-3-yl)piperidine-2,6-dione (HCB74)

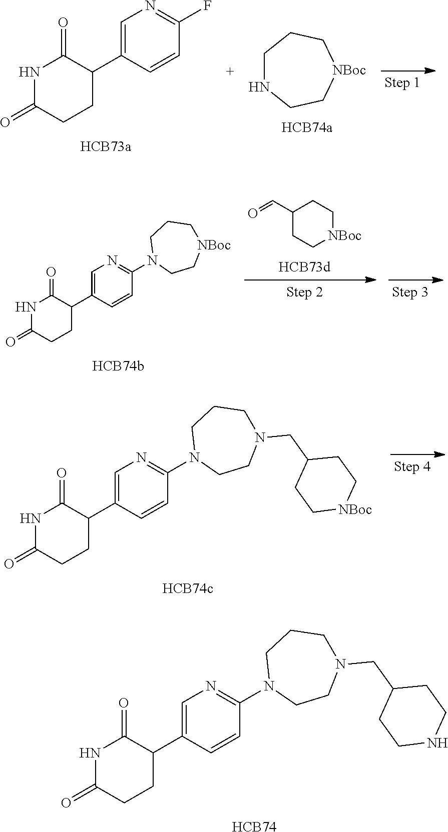

### Step 1: Synthesis of tert-butyl 4-(5-(2,6-dioxopiperidin-3-yl)pyridin-2-yl)-1,4-diazepane-1-carboxylate

To a 20 mL microwave reaction vial was added rac-(3R)-3-(6-fluoropyridin-3-yl)piperidine-2,6-dione (500.00 mg, 2.40 mmol), tert-butyl 1,4-diazepane-1-carboxylate (962.01 mg, 4.80 mmol), N,N-diisopropylethylamine (1.68 mL, 1.24 g, 9.61 mmol), and DMSO (5.00 mL). The reaction mixture was stirred at 150 C by MW for 16 h. The reaction mixture was concentrated, and the resulting residue was used in the next step without further purification. LCMS: C₂₀H₂₈N₄O₄ requires: 388.2, found: m/z=389.3 [M+H]⁺.

### Step 2: Synthesis of 3-(6-(1,4-diazepan-1-yl)pyridin-3-yl)piperidine-2,6-dione

To a 20 mL vial was added rac-tert-butyl 4-{5-[(3R)-2,6-dioxopiperidin-3-yl]pyridin-2-yl}-1,4-diazepane-1-carboxylate (932.00 mg, 2.40 mmol) and DCM (3 mL). To the reaction mixture was added trifluoroacetic acid (2.77 mL, 4.10 g, 35.99 mmol). After 30 min, reaction mixture was concentrated under reduced pressure. The resulting residue was purified by reverse phase column chromatography (100 g C18 silica, 0-30% MeCN+0.1% TFA/H₂O+0.1% TFA) to yield the title compound as a white solid (235 mg, 34%). LCMS: C₁₅H₂₀N₄O₂ requires: 288.2, found: m/z=289.3 [M+H]⁺.

### Step 3: Synthesis of tert-butyl 4-((4-(5-(2,6-dioxopiperidin-3-yl)pyridin-2-yl)-1,4-diazepan-1-yl)methyl)piperidine-1-carboxylate

To a 2 dram vial was added rac-(3R)-3-[6-(1,4-diazepan-1-yl)pyridin-3-yl]piperidine-2,6-dione (20.00 mg, 0.07 mmol), tert-butyl 4-formylpiperidine-1-carboxylate (16.27 mg, 0.08 mmol), DCM (0.50 mL), and DMSO (0.10 mL). To the reaction mixture was added sodium triacetoxyborohydride (44.10 mg, 0.21 mmol) in one portion. After 1 h, the reaction mixture was quenched with sat. NaHCO₃(aq). The product was extracted 3× w/DCM. The combined organic layers were dried over MgSO4, then concentrated. The resulting residue was purified by column chromatography (4 g silica, 0-15% MeOH/DCM) to yield the title compound as a white solid (25 mg, 75%). LCMS: C₂₆H₃₉N₅O₄ requires: 485.3, found: m/z=486.3 [M+H]⁺.

### Step 4: Synthesis of 3-(6-(4-(piperidin-4-ylmethyl)-1,4-diazepan-1-yl)pyridin-3-yl)piperidine-2,6-dione (HCB74)

To a 20 mL vial was added rac-tert-butyl 4-[(4-{5-[(3R)-2,6-dioxopiperidin-3-yl]pyridin-2-yl}-1,4-diazepan-1-yl)methyl]piperidine-1-carboxylate (25.00 mg, 0.05 mmol) and DCM (0.30 mL). To the mixture was added 4N hydrogen chloride in dioxane (1.34 mL, 0.20 g, 5.37 mmol) in a dropwise fashion. After 1 h, the reaction mixture was concentrated under reduced pressure to yield the title compound as a white solid (20 mg, 100%). LCMS: C₂₁H₃₁N₅O₂ requires: 385.2, found: m/z=386.3 [M+H]⁺.

### Example 70. 2,6-bis(benzyloxy)-2′-fluoro-3,4′-bipyridine (HCB75)

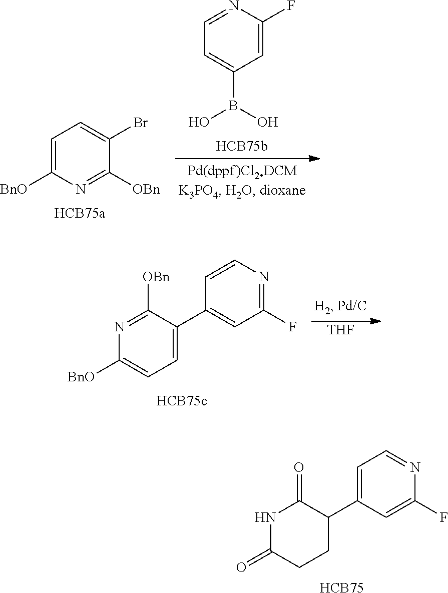

### Step 1: Synthesis of 3-(2-fluoropyridin-4-yl)piperidine-2,6-dione

Into a 5-L 4-necked round-bottom flask purged and maintained with an inert atmosphere of nitrogen, was placed 2,6-bis(benzyloxy)-3-bromopyridine (408 g, 1101.97 mmol, 1.00 equiv), dioxane (4080 mL), water (816 mL), K₃PO₄ (701.73 g, 3305.91 mmol, 3.00 equiv), 2-fluoropyridin-4-ylboronic acid (163.04 g, 1157.06 mmol, 1.05 equiv), Pd(dppf)C₁₂ (40.32 g, 55.09 mmol, 0.05 equiv). The resulting solution was stirred for 3 h at 100° C. The mixture was allowed to cool down to RT. The solids were filtered out. The resulting mixture was concentrated. The residue was applied onto a silica gel column with ethyl acetate/petroleum ether (8%). This resulted in 2,6-bis(benzyloxy)-2′-fluoro-3,4′-bipyridine as a white solid (310 g, 72.8%). LCMS: C₂₄H₁₉FN₂O₂ requires: 386, found: m/z=387 [M+H]⁺.

### Step 2: Synthesis of 2,6-bis(benzyloxy)-2′-fluoro-3,4′-bipyridine (HCB75)

Into a 10-L 4-necked round-bottom flask, was placed 2,6-bis(benzyloxy)-2′-fluoro-3,4′-bipyridine (340 g, 879.85 mmol, 1.00 equiv), THE (6800 mL), Pd/C (34 g, 319.48 mmol, 0.36 equiv). To the above H₂ (gas) was introduced to keep pressure about 3 atm. The resulting solution was stirred for overnight at room temperature. After 16 h, additional Pd/C (10 g, 93.96 mmol, 0.11 equiv) was added, the resulting solution was stirred 30 h under the same condition as before. Then the solids were filtered out. The resulting mixture was concentrated. This resulted in 3-(2-fluoropyridin-4-yl)piperidine-2,6-dione as a pale yellow solid (103 g, 56.0%). LCMS: C₁₀H₉FN₂O₂ requires: 208, found: m/z=209 [M+H]⁺.

### Example 71. 3-(2-(4-(piperidin-4-ylmethyl)-1,4-diazepan-1-yl)pyridin-4-yl)piperidine-2,6-dione (HCB76)

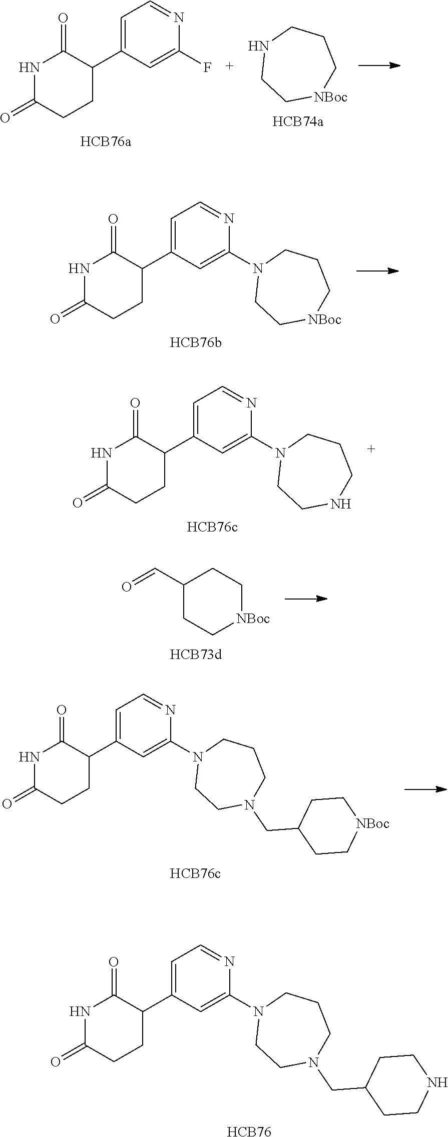

### Step 1: Synthesis of tert-butyl 4-(4-(2,6-dioxopiperidin-3-yl)pyridin-2-yl)-1,4-diazepane-1-carboxylate

To a 40 mL vial was added rac-(3R)-3-(2-fluoropyridin-4-yl)piperidine-2,6-dione (1000.00 mg, 4.80 mmol), tert-butyl 1,4-diazepane-1-carboxylate (1443.01 mg, 7.20 mmol), N,N-diisopropylethylamine (3.36 mL, 2.48 g, 19.21 mmol), and DMSO (7.00 mL). The reaction mixture was stirred at 115 C for 3 d. The reaction mixture was then diluted with EtOAc and brine. The product was extracted with EtOAc (2×). The combined organic layers were washed with brine (3×), dried over MgSO4, then concentrated. The resulting residue was purified by column chromatography (80 g silica, 0-80% EtOAc/DCM) to yield the title compound as a light brown solid (1.04 g, 56%). LCMS: C₂₀H₂₈N₄O₄ requires: 388.2, found: m/z=389.3 [M+H]⁺.

### Step 2: Synthesis of 3-(2-(1,4-diazepan-1-yl)pyridin-4-yl)piperidine-2,6-dione

To a 40 mL vial was added rac-tert-butyl 4-{4-[(3R)-2,6-dioxopiperidin-3-yl]pyridin-2-yl}-1,4-diazepane-1-carboxylate (1039.00 mg, 2.67 mmol) and DCM (2.00 mL). To the reaction mixture was added 4 M hydrogen chloride in dioxane (6.69 mL, 0.98 g, 26.75 mmol). After 1 h, the reaction mixture was concentrated to yield the title compound as a white solid (952 mg, quantitative yield). LCMS: C₁₅H₂₀N₄O₂ requires: 288.2, found: m/z=289.1 [M+H]⁺.

### Step 3: Synthesis of tert-butyl 4-((4-(4-(2,6-dioxopiperidin-3-yl)pyridin-2-yl)-1,4-diazepan-1-yl)methyl)piperidine-1-carboxylate

To a 2 dram vial was added rac-(3R)-3-[2-(1,4-diazepan-1-yl)pyridin-4-yl]piperidine-2,6-dione hydrochloride (38.30 mg, 0.12 mmol), tert-butyl 4-formylpiperidine-1-carboxylate (25.15 mg, 0.12 mmol), DCM (1.00 mL), and triethylamine (0.11 mL, 0.08 g, 0.83 mmol). To the reaction mixture was added 1,1-bis(acetyloxy)-3-oxo-1lambda5,2-benziodaoxol-1-yl acetate (0.15 g, 0.35 mmol) in one portion. After 1 h, the reaction mixture was quenched with NaHCO₃(aq). The product was extracted with DCM (3×), dried over MgSO4, then concentrated. The resulting residue was purified by FC (4 g silica, 0-10% MeOH/DCM) to yield the title compound as a white solid (50 mg, 88%). LCMS: C₂₆H₃₉N₅O₄ requires: 485.3, found: m/z=486.4 [M+H]⁺.

### Step 4: Synthesis of 3-(2-(4-(piperidin-4-ylmethyl)-1,4-diazepan-1-yl)pyridin-4-yl)piperidine-2,6-dione (HCB76)

To a 20 mL vial was added rac-tert-butyl 4-[(4-{4-[(3R)-2,6-dioxopiperidin-3-yl]pyridin-2-yl}-1,4-diazepan-1-yl)methyl]piperidine-1-carboxylate (50.30 mg, 0.10 mmol) and DCM (0.50 mL). To the reaction mixture was added 4N hydrogen chloride in dioxane (2.33 mL, 0.34 g, 9.32 mmol). After 1 h, the reaction mixture was concentrated under reduced pressure to yield the title compound as a white solid (75.6 mg, quantitative yield). LCMS: C₂₁H₃₁N₅O₂ requires: 385.2, found: m/z=386.2 [M+H]⁺.

### Example 72. 3-(1,2,3,4-tetrahydroisoquinolin-6-yl)piperidine-2,6-dione (HCB77)

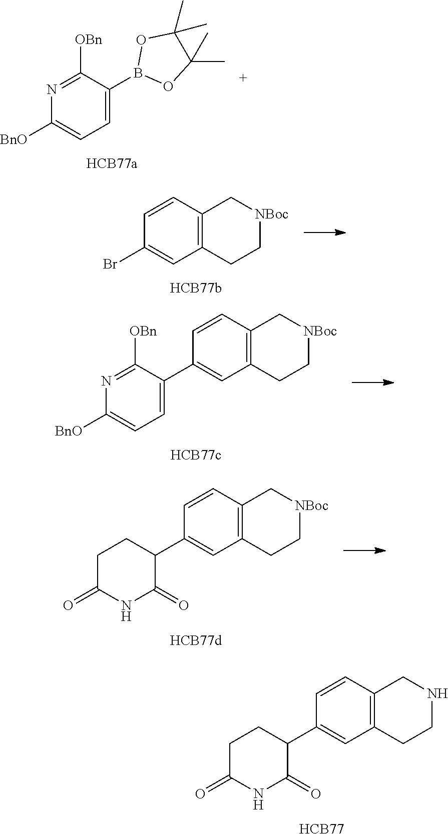 [SMILES: CC1(C)OB(c2ccc(OCc3ccccc3)nc2OCc2ccccc2)OC1(C)C ; CC(C)(C)OC(=O)N1CCc2cc(Br)ccc2C1 ; CC(C)(C)OC(=O)N1CCc2cc(-c3ccc(OCc4ccccc4)nc3OCc3ccccc3)ccc2C1 ; CC(C)(C)OC(=O)N1CCc2cc(C3CCC(=O)NC3=O)ccc2C1 ; "HCB77" ; O=C1CCC(c2ccc3c(c2)CCNC3)C(=O)N1]

### Step 1: Synthesis of tert-butyl 6-bromo-3,4-dihydroisoquinoline-2(1H)-carboxylate

To a 40 mL vial was added tert-butyl 6-bromo-3,4-dihydro-1H-isoquinoline-2-carboxylate (1000.00 mg, 3.20 mmol), 2,6-bis(benzyloxy)-3-(4,4,5,5-tetramethyl-1,3,2-dioxaborolan-2-yl)pyridine (1470.31 mg, 3.52 mmol), tripotassium phosphate (2.04 g, 9.61 mmol), Pd(dppf)C₁₂·DCM (0.26 g, 0.32 mmol), dioxane (7.00 mL),and water (2.50 mL). The reaction mixture was degassed with nitrogen for 15 min, then stirred at 90 C for 16 h. The reaction mixture was then diluted with water and EtOAc, then filtered through celite. The product was extracted with EtOAc (3×), dried over MgSO₄, then concentrated. The resulting residue was purified by FC (80 g silica, 0-25% EtOAc/hexanes) to yield the title compound as a colorless oil (1.48 g, 88%). LCMS: C₃₃H₃₄N₂O₄ requires: 522.3, found: m/z=523.3 [M+H]⁺.

### Step 2: Synthesis of tert-butyl 6-(2,6-bis(benzyloxy)pyridin-3-yl)-3,4-dihydroisoquinoline-2(1H)-carboxylate

To a 40 mL vial was added tert-butyl 6-[2,6-bis(benzyloxy)pyridin-3-yl]-3,4-dihydro-1H-isoquinoline-2-carboxylate (1480.00 mg, 2.83 mmol), Pd/C (700.00 mg), THF (10.00 mL), and EtOH (10.00 mL). The reaction mixture was sparged with hydrogen for 5 min, then the reaction was stirred under hydrogen atmosphere (balloon) for 16 h. The reaction mixture was filtered through celite, then concentrated. The resulting residue was purified by FC (40 g silica, 0-10% MeOH/DCM) to yield the title compound as a white solid (852 mg, 87%). LCMS: C₁₉H₂₄N₂O₄ requires: 344.2, found: m/z=345.2 [M+H]⁺.

### Step 3: Synthesis of tert-butyl 6-(2,6-dioxopiperidin-3-yl)-3,4-dihydroisoquinoline-2(1H)-carboxylate (HCB77)

To a 40 mL vial was added rac-tert-butyl 6-[(3R)-2,6-dioxopiperidin-3-yl]-3,4-dihydro-1H-isoquinoline-2-carboxylate (852.00 mg, 2.47 mmol) and DCM (2.00 mL). To the reaction mixture was added 4 M hydrogen chloride in dioxane (6.18 mL, 0.90 g, 24.74 mmol) in a dropwise fashion. After 1 h, the reaction mixture was concentrated to yield the title compound as a white solid (707 mg, quantitative yield). LCMS: C₁₄H₁₆N₂O₂ requires: 244.1, found: m/z=245.0 [M+H]⁺.

### Example 73. 3-(2-(piperidin-4-ylmethyl)-1,2,3,4-tetrahydroisoquinolin-6-yl)piperidine-2,6-dione (HCB78)

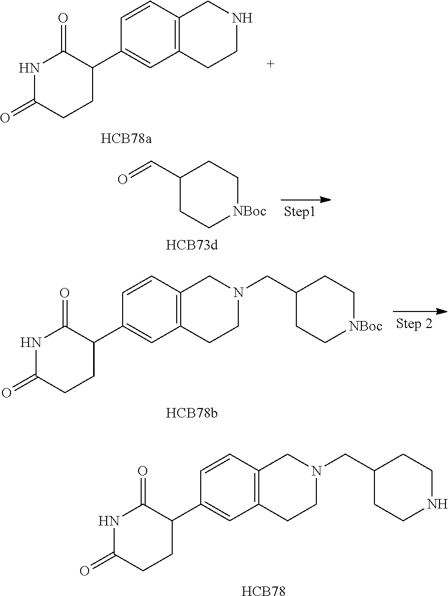

### Step 1: Synthesis of tert-butyl 4-((6-(2,6-dioxopiperidin-3-yl)-3,4-dihydroisoquinolin-2(1H)-yl)methyl)piperidine-1-carboxylate

To a 20 mL vial was added rac-(3R)-3-(1,2,3,4-tetrahydroisoquinolin-6-yl)piperidine-2,6-dione hydrochloride (150.00 mg, 0.53 mmol), tert-butyl 4-formylpiperidine-1-carboxylate (113.95 mg, 0.53 mmol), triethylamine (0.52 mL, 0.38 g, 3.74 mmol), DMSO (2.00 mL) and DCM (4.00 mL). To the reaction mixture was added sodium triacetoxyborohydride (0.34 g, 1.60 mmol). The reaction mixture was stirred for 1 h, then quenched with sat. NaHCO₃(aq). The product was extracted with DCM (3×), dried over MgSO4, then concentrated. The resulting residue was purified by FC (24 g silica, 0-10% MeOH/DCM) to yield the title compound as a light purple amorphous solid (188 mg, 80%). LCMS: C₂₅H₃₅N₃O₄ requires: 441.3, found: m/z=442.3 [M+H]⁺.

### Step 2: Synthesis of 3-(2-(piperidin-4-ylmethyl)-1,2,3,4-tetrahydroisoquinolin-6-yl)piperidine-2,6-dione (HCB78)

To a 20 mL vial was added rac-tert-butyl 4-({6-[(3R)-2,6-dioxopiperidin-3-yl]-3,4-dihydro-1H-isoquinolin-2-yl}methyl)piperidine-1-carboxylate (187.90 mg, 0.43 mmol) and DCM (1.00 mL). To this solution was added 4N hydrogen chloride in dioxane (2.13 mL, 0.31 g, 8.51 mmol). After 2 h, the reaction mixture was concentrated under reduced pressure to yield the title compound as a white solid (194.2 mg, quantitative yield). LCMS: C₂₀H₂₇N₃O₂ requires: 341.2, found: m/z=342.2 [M+H]⁺.

### Example 74. 3-(2-(piperidin-4-ylmethyl)-1,2,3,4-tetrahydroisoquinolin-7-yl)piperidine-2,6-dione (HCB79)

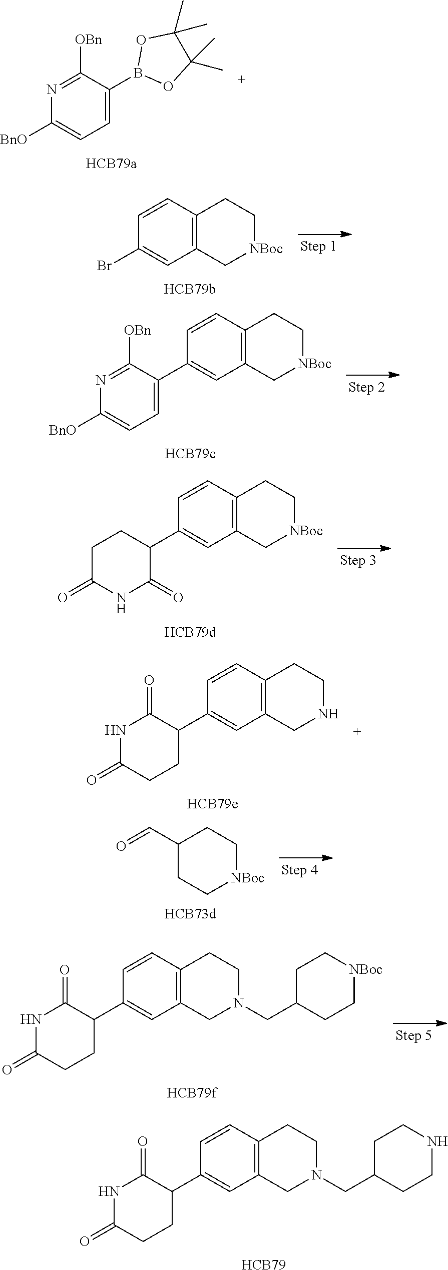

### Step 1: Synthesis of tert-butyl 7-(2,6-bis(benzyloxy)pyridin-3-yl)-3,4-dihydroisoquinoline-2(1H)-carboxylate

To a 40 mL vial was added tert-butyl 7-bromo-3,4-dihydro-1H-isoquinoline-2-carboxylate (1000.00 mg, 3.20 mmol), 2,6-bis(benzyloxy)-3-(4,4,5,5-tetramethyl-1,3,2-dioxaborolan-2-yl)pyridine (1470.31 mg, 3.52 mmol), Pd(dppf)Cl2 (0.26 g, 0.32 mmol), tripotassium phosphate (2.04 g, 9.61 mmol), water (2.50 mL), and dioxane (7.00 mL). The reaction mixture was stirred at 90 C for 16 h. The reaction mixture was diluted with EtOAc and brine. The product was extracted with EtOAc (2×), dried over MgSO4, then concentrated. The resulting residue was purified by FC (80 g silica, 0-25% EtOAc/hex) to yield the title compound as a colorless amorphous solid (1.31 g, 78%). LCMS: C₃₃H₃₄N₂O₄ requires: 522.3, found: m/z=523.3 [M+H]⁺.

### Step 2: Synthesis of tert-butyl 7-(2,6-dioxopiperidin-3-yl)-3,4-dihydroisoquinoline-2(1H)-carboxylate

To a 40 mL vial was added tert-butyl 7-[2,6-bis(benzyloxy)pyridin-3-yl]-3,4-dihydro-1H-isoquinoline-2-carboxylate (1310.00 mg, 2.51 mmol), Pd/C (650.00 mg), EtOH (8.00 mL), and THE (8.00 mL). The reaction mixture was sparged with hydrogen for 5 min, then stirred under hydrogen atmosphere (balloon) for 16 h. The reaction mixture was concentrated, and the resulting residue was purified by FC (40 g silica, 0-10% MeOH/DCM) to yield the title compound as a white solid (1.04 g, quantitative yield). LCMS: C₁₉H₂₄N₂O₄ requires: 344.2, found: m/z=367.1 [M+Na]⁺.

### Step 3: Synthesis of 3-(1,2,3,4-tetrahydroisoquinolin-7-yl)piperidine-2,6-dione

To a 40 mL vial was added rac-tert-butyl 7-[(3R)-2,6-dioxopiperidin-3-yl]-3,4-dihydro-1H-isoquinoline-2-carboxylate (1040.00 mg, 3.02 mmol) and DCM (2.00 mL). To the reaction mixture was added 4N hydrogen chloride in dioxane (7.55 mL, 1.10 g, 30.20 mmol). After 2 h, the reaction mixture was concentrated to yield the title compound as a white solid (829.0 mg, quantitative yield). LCMS: C₁₄H₁₆N₂O₂ requires: 244.1, found: m/z=245.3 [M+H]⁺.

### Step 4: Synthesis of tert-butyl 4-((7-(2,6-dioxopiperidin-3-yl)-3,4-dihydroisoquinolin-2(1H)-yl)methyl)piperidine-1-carboxylate

To a 20 mL vial was added rac-(3R)-3-(1,2,3,4-tetrahydroisoquinolin-7-yl)piperidine-2,6-dione hydrochloride (150.00 mg, 0.53 mmol), tert-butyl 4-formylpiperidine-1-carboxylate (113.95 mg, 0.53 mmol), triethylamine (0.52 mL, 0.38 g, 3.74 mmol), DCM (4.00 mL), and DMSO (1.00 mL). To the reaction mixture was added sodium triacetoxyborohydride (0.34 g, 1.60 mmol). The reaction mixture was stirred for 1 h, then quenched with sat. NaHCO₃(aq). The product was extracted with DCM (3×), dried over MgSO4, then concentrated. The resulting residue was purified by FC (24 g silica, 0-10% MeOH/DCM) to yield the title compound as a white solid (246 mg, quantitative yield). LCMS: C₂₅H₃₅N₃O₄ requires: 441.3, found: m/z=442.3 [M+H]⁺.

### Step 5: Synthesis of 3-(2-(piperidin-4-ylmethyl)-1,2,3,4-tetrahydroisoquinolin-7-yl)piperidine-2,6-dione (HCB79)

To a 20 mL vial was added rac-tert-butyl 4-({7-[(3R)-2,6-dioxopiperidin-3-yl]-3,4-dihydro-1H-isoquinolin-2-yl}methyl)piperidine-1-carboxylate (245.50 mg, 0.56 mmol) and DCM (1.00 mL). To the reaction mixture was added 4N hydrogen chloride in dioxane (2.08 mL, 0.30 g, 8.34 mmol). After 2 h, the reaction mixture was concentrated under reduced pressure to yield the title compound as a white solid (194.2 mg, quantitative yield). LCMS: C₂₀H₂₇N₃O₂ requires: 341.2, found: m/z=342.3 [M+H]⁺.

### Example 75. 3-(1-(piperidin-4-ylmethyl)-1,2,3,4-tetrahydroquinolin-6-yl)piperidine-2,6-dione (HCB80)

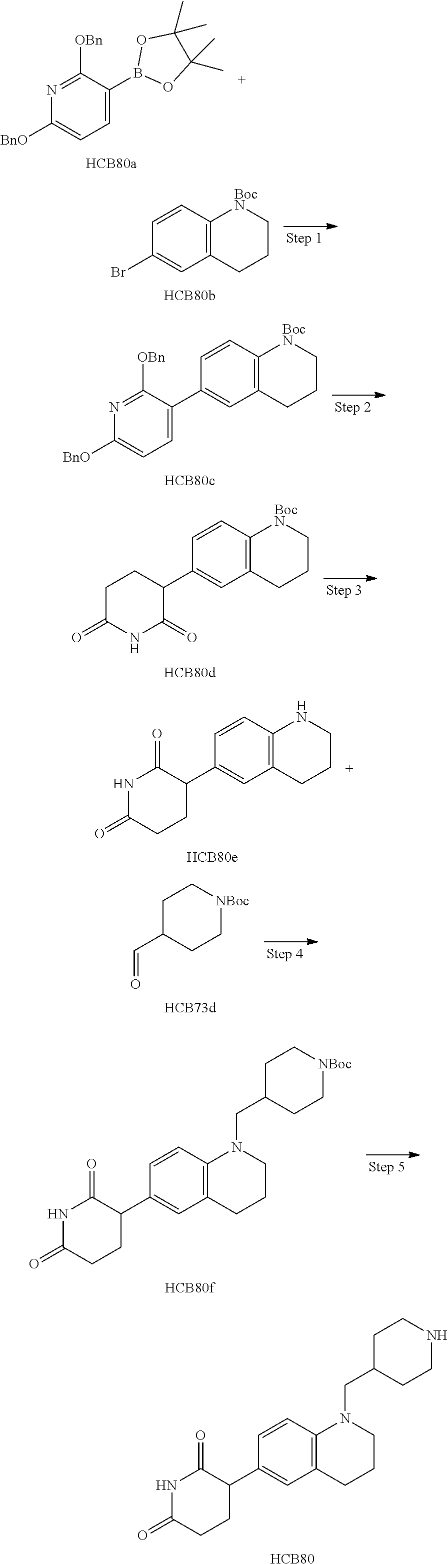

### Step 1: Synthesis of tert-butyl 6-(2,6-bis(benzyloxy)pyridin-3-yl)-3,4-dihydroquinoline-1(2H)-carboxylate

To a 40 mL vial was added tert-butyl 6-bromo-3,4-dihydro-2H-quinoline-1-carboxylate (1000.00 mg, 3.20 mmol), 2,6-bis(benzyloxy)-3-(4,4,5,5-tetramethyl-1,3,2-dioxaborolan-2-yl)pyridine (1470.31 mg, 3.52 mmol), Pd(dppf)C₁₂*DCM (0.26 g, 0.32 mmol), tripotassium phosphate (2.04 g, 9.61 mmol), dioxane (7.00 mL), and water (2.50 mL). The reaction mixture was sparged with nitrogen for 10 min, then stirred at 90 C for 2 d. The reaction mixture was then diluted with water and EtOAc, then filtered through celite. The product was extracted with EtOAc, dried over MgSO4, then concentrated. The resulting residue was purified by column chromatography (80 g silica, 0-30% EtOAc/hex) to yield the title compound as an off-white amorphous solid (1.56 g, 93%). LCMS: C₃₃H₃₄N₂O₄ requires: 522.3, found: m/z=523.3 [M+H]⁺.

### Step 2: Synthesis of tert-butyl 6-(2,6-dioxopiperidin-3-yl)-3,4-dihydroquinoline-1(2H)-carboxylate

To a 40 mL vial was added tert-butyl 6-[2,6-bis(benzyloxy)pyridin-3-yl]-3,4-dihydro-2H-quinoline-1-carboxylate (1560.00 mg, 2.98 mmol), Pd/C (650.00 mg), EtOH (8.00 mL), and THE (8.00 mL). The reaction mixture was sparged with H₂ for 30 min before stirring under H₂ atmosphere for 16 h. The reaction mixture was then filtered through celite with DCM, then concentrated. The resulting residue was purified by FC (40 g silica, 0-10% MeOH/DCM) to yield the title compound as a white solid (976 mg, 95%). LCMS: C₁₉H₂₄N₂O₄ requires: 344.2, found: m/z=367.1 [M+Na]⁺.

### Step 3: Synthesis of 3-(1,2,3,4-tetrahydroquinolin-6-yl)piperidine-2,6-dione

To a 40 mL vial was added tert-butyl 6-(2,6-dioxopiperidin-3-yl)-3,4-dihydro-2H-quinoline-1-carboxylate (976.00 mg, 2.83 mmol) and dioxane (2.00 mL). To the slurry was added 4N hydrogen chloride in dioxane (7.08 mL, 1.03 g, 28.34 mmol). The reaction mixture was stirred for 2 h, then concentrated as a white solid (819.8 mg, quantitative yield). LCMS: C₁₄H₁₆N₂O₂ requires: 244.1, found: m/z=245.4 [M+H]⁺.

### Step 4: Synthesis of tert-butyl 4-((6-(2,6-dioxopiperidin-3-yl)-3,4-dihydroquinolin-1(2H)-yl)methyl)piperidine-1-carboxylate

To a 20 mL vial was added 3-(1,2,3,4-tetrahydroquinolin-6-yl)piperidine-2,6-dione hydrochloride (150.00 mg, 0.53 mmol), tert-butyl 4-formylpiperidine-1-carboxylate (113.95 mg, 0.53 mmol), triethylamine (0.52 mL, 0.38 g, 3.74 mmol), and DCM (4.00 mL). To the reaction mixture was added sodium triacetoxyborohydride (339.71 mg, 1.60 mmol). The reaction mixture was stirred for 1 h, then quenched with sat. NaHCO₃ (aq). The product was extracted with DCM (2×), dried over MgSO4, then concentrated. The resulting residue was purified by FC (24 g silica, 0-10% MeOH/DCM) to yield the title compound as a white solid (226 mg, 96%). LCMS: C₂₅H₃₅N₃O₄ requires: 441.3, found: m/z=442.3 [M+H]⁺.

### Step 5: Synthesis of 3-(1-(piperidin-4-ylmethyl)-1,2,3,4-tetrahydroquinolin-6-yl)piperidine-2,6-dione (HCB80)

To a 20 mL vial was added rac-tert-butyl 4-({6-[(3R)-2,6-dioxopiperidin-3-yl]-3,4-dihydro-2H-quinolin-1-yl}methyl)piperidine-1-carboxylate (226.10 mg, 0.51 mmol) and DCM (1.00 mL). To the reaction mixture was added 4N hydrogen chloride in dioxane (1.92 mL, 0.28 g, 7.68 mmol). Stirred for 1 h, then the reaction mixture was concentrated. After 2 h, the reaction mixture was concentrated under reduced pressure to yield the title compound as a white solid (247 mg, quantitative yield). LCMS: C₂₀H₂₇N₃O₂ requires: 341.2, found: m/z=342.3 [M+H]⁺.

### Example 76. 1-(3-(2,4-dioxotetrahydropyrimidin-1(2H)-yl)-4-fluorobenzoyl)piperidine-4-carbaldehyde (HCB81)

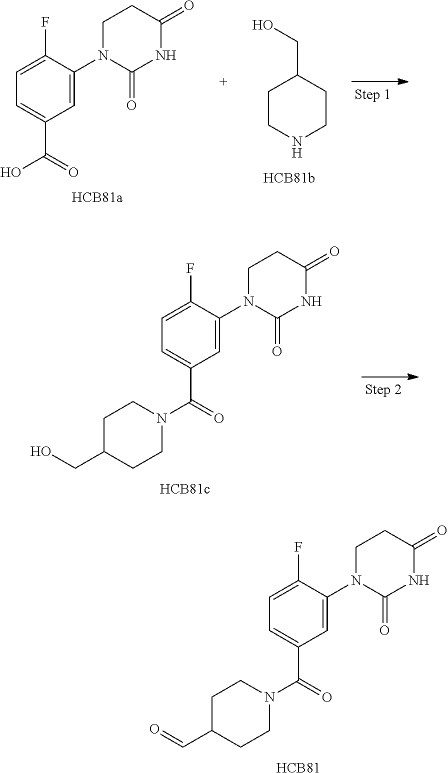 [SMILES: O=C1CCN(c2cc(C(=O)O)ccc2F)C(=O)N1 ; OCC1CCNCC1 ; O=C1CCN(c2cc(C(=O)N3CCC(CO)CC3)ccc2F)C(=O)N1 ; "HCB81" ; O=CC1CCN(C(=O)c2ccc(F)c(N3CCC(=O)NC3=O)c2)CC1]

### Step 1: Synthesis of 1-(2-fluoro-5-(4-(hydroxymethyl)piperidine-1-carbonyl)phenyl)dihydropyrimidine-2,4(1H,3H)-dione

To a 20 mL vial was added 3-(2,4-dioxo-1,3-diazinan-1-yl)-4-fluorobenzoic acid (150.00 mg, 0.59 mmol), piperidin-4-ylmethanol (68.50 mg, 0.59 mmol), N,N-diisopropylethylamine (0.31 mL, 0.23 g, 1.78 mmol), and DMF (2 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (226.15 mg, 0.59 mmol) in DMF (1 mL). The reaction mixture was stirred for 16 h, and the crude reaction mixture was purified by RP-FC (100 g C18 silica, 0-30% MeCN/water+0.1% TFA) to yield the title compound as a white solid (263 mg, quantitative yield). LCMS: C₁₇H₂₀FN₃O₄ requires: 349.1, found: m/z=350.2 [M+H]⁺.

### Step 2: Synthesis of 1-(3-(2,4-dioxotetrahydropyrimidin-1(2H)-yl)-4-fluorobenzoyl)piperidine-4-carbaldehyde (HCB81)

To a 2 dram vial was added 1-{2-fluoro-5-[4-(hydroxymethyl)piperidine-1-carbonyl]phenyl}-1,3-diazinane-2,4-dione (40.00 mg, 0.11 mmol), DMSO (0.50 mL), DCM (1.50 mL) 0, and IBX polystrene (1.22 mmol/g, 0.3 g). The reaction mixture was stirred for 1 d, then filtered with additional 20 mL DCM. The crude mixture was concentrated and carried forward to the next step as a solution in DMSO without further purification. LCMS: C₁₇H₁₈FN₃O₄ requires: 347.1, found: m/z=348.3 [M+H]⁺.

### Example 77. 1-(4-(2,4-dioxotetrahydropyrimidin-1(2H)-yl)phenyl)piperidine-4-carbaldehyde (HCB82)

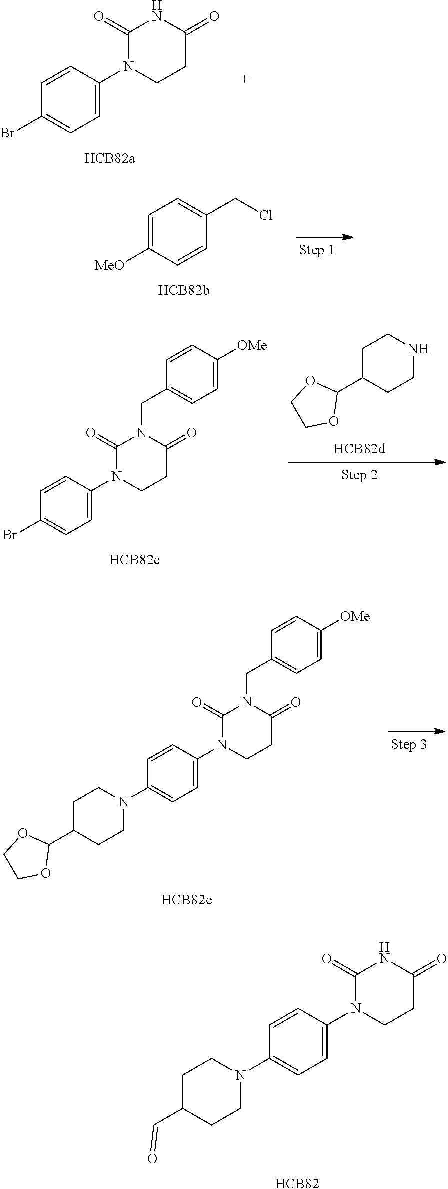

### Step 1: 1-(4-bromophenyl)-3-(4-methoxybenzyl)dihydropyrimidine-2,4(1H,3H)-dione (HCB82c)

To a 40 mL vial was added 1-(4-bromophenyl)-1,3-diazinane-2,4-dione (6400.00 mg, 23.78 mmol), caesium carbonate (9.30 g, 28.54 mmol), and DMF (60.00 mL). To the reaction mixture was added 4-methoxybenzyl chloride (4.17 mL, 4842.08 mg, 30.92 mmol). The reaction mixture was stirred overnight, then quenched with water. The product was extracted with DCM (3×), dried over Na₂SO₄, then concentrated. The resulting residue was purified by FC (330 g silica, 0-50% EtOAc/DCM). The resulting purified residue was concentrated under reduced pressure into an amorphous solid and triturated with hexanes to yield the title compound a white solid (9.26 g, quantitative yield). LCMS: C₁₈H₁₇BrN₂O₃ requires: 388.0, found: m/z=389.2 [M+H]⁺.

### Step 2: Synthesis of 1-(4-(4-(1,3-dioxolan-2-yl)piperidin-1-yl)phenyl)-3-(4-methoxybenzyl)dihydropyrimidine-2,4(1H,3H)-dione

To a 2 dram vial was added 1-(4-bromophenyl)-3-[(4-methoxyphenyl)methyl]-1,3-diazinane-2,4-dione (550.00 mg, 1.41 mmol), 4-(1,3-dioxolan-2-yl)piperidine (666.42 mg, 4.24 mmol), di-tert-butyl({2′,4′,6′-triisopropyl-[1,1′-biphenyl]-2-yl})phosphane (240.01 mg, 0.57 mmol), (acetyloxy)palladio acetate (63.45 mg, 0.28 mmol), caesium carbonate (1.38 g, 4.24 mmol), and dioxane (5.00 mL). The reaction mixture was sparged with N₂ for 10 min, then stirred at 95 C overnight. The crude reaction mixture was diluted with DCM and water, then filtered through celite. The product was extracted with DCM (3×), dried over MgSO4, then concentrated. The resulting residue was purified by FC (40 g silica, 0-100% EtOAc/hex) to yield the title compound as a white solid (395 mg, 60%). LCMS: C₂₆H₃₁N₃O₅ requires: 465.2, found: m/z=466.4 [M+H]⁺.

### Step 3: Synthesis of 1-(4-(2,4-dioxotetrahydropyrimidin-1(2H)-yl)phenyl)piperidine-4-carbaldehyde (HCB82)

To a 20 mL vial was added 1-{4-[4-(1,3-dioxolan-2-yl)piperidin-1-yl]phenyl}-3-[(4-methoxyphenyl)methyl]-1,3-diazinane-2,4-dione (395.00 mg, 0.85 mmol) and TFA (5.00 mL). To the reaction mixture was added trifluoromethanesulfonic acid (374.49 uL, 636.64 mg, 4.24 mmol). Full conversion after 10 min. The reaction mixture was diluted with minimal water, then purified by RP-FC (415 g C18 silica, 0-50% MeCN/water+0.1% TFA) to yield the title compound as a white solid (40 mg, 16%). LCMS: C₁₆H₂₁N₃O₃ requires: 301.1, found: m/z=302.2 [M+H]⁺.

### Example 78. 1-(4-(2,4-dioxotetrahydropyrimidin-1(2H)-yl)phenyl)pyrrolidine-3-carbaldehyde (HCB83)

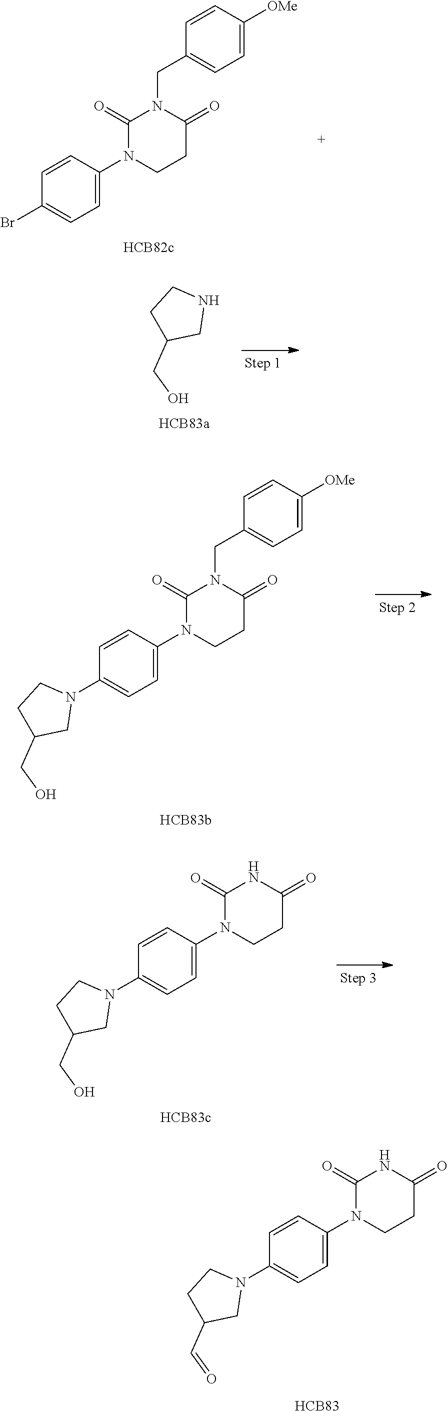 [SMILES: COc1ccc(CN2C(=O)CCN(c3ccc(Br)cc3)C2=O)cc1 ; OCC1CCNC1 ; COc1ccc(CN2C(=O)CCN(c3ccc(N4CCC(CO)C4)cc3)C2=O)cc1 ; O=C1CCN(c2ccc(N3CCC(CO)C3)cc2)C(=O)N1 ; "HCB83" ; O=CC1CCN(c2ccc(N3CCC(=O)NC3=O)cc2)C1]

### Step 1: Synthesis of 1-(4-(3-(hydroxymethyl)pyrrolidin-1-yl)phenyl)-3-(4-methoxybenzyl)dihydropyrimidine-2,4(1H,3H)-dione

To a 40 mL vial was added 1-(4-bromophenyl)-3-[(4-methoxyphenyl)methyl]-1,3-diazinane-2,4-dione (500.00 mg, 1.28 mmol), rac-(3R)-pyrrolidin-3-ylmethanol (259.86 mg, 2.57 mmol), caesium carbonate (1.26 g, 3.85 mmol), (acetyloxy)palladio acetate (57.68 mg, 0.26 mmol), di-tert-butyl({2′,4′,6′-triisopropyl-[1,1′-biphenyl]-2-yl})phosphane (0.22 g, 0.51 mmol), and dioxane (5.00 mL). The reaction mixture was stirred at 90 C overnight. The reaction mixture was diluted with water, and the product was extracted with EtOAc (3×), dried over MgSO4, then concentrated. The resulting residue was purified by FC (24 g silica, 0-100% DCM/EtOAc) to yield the title compound as an off-white solid (295 mg, 56%). LCMS: C₂₃H₂₇N₃O₄ requires: 409.2, found: m/z=410.3 [M+H]⁺.

### Step 2: Synthesis of 1-(4-(3-(hydroxymethyl)pyrrolidin-1-yl)phenyl)dihydropyrimidine-2,4(1H,3H)-dione

To a 40 mL vial was added rac-1-{4-[(3R)-3-(hydroxymethyl)pyrrolidin-1-yl]phenyl}-3-[(4-methoxyphenyl)methyl]-1,3-diazinane-2,4-dione (645.00 mg, 1.58 mmol) and TFA (10.00 mL). To the reaction mixture was added trifluoromethanesulfonic acid (0.70 mL, 1.18 g, 7.88 mmol). The reaction mixture was stirred for 1.5 h, then diluted with water (˜5 mL). The resulting mixture was purified by RP-FC (400 g C18 silica, 0-50% MeCN/water+0.1% TFA) to yield the title compound as a white solid (513 mg, quantitative yield). LCMS: C₁₅H₁₉N₃O₃ requires: 289.1, found: m/z=290.2 [M+H]⁺.

### Step 3: Synthesis of 1-(4-(2,4-dioxotetrahydropyrimidin-1(2H)-yl)phenyl)pyrrolidine-3-carbaldehyde (HCB83)

To a 40 mL vial was added rac-1-{4-[(3R)-3-(hydroxymethyl)pyrrolidin-1-yl]phenyl}-1,3-diazinane-2,4-dione (456.00 mg, 1.58 mmol), DCM (7.50 mL) and DMSO (2.50 mL). The reaction mixture was cooled to 0° C., then sulfur trioxide pyridine complex (2508.41 mg, 15.76 mmol) was added to the reaction mixture. The reaction mixture was stirred warming to room temperature for 1 h; full conversion by LCMS. After 2 h, the reaction mixture was concentrated, and the resulting residue was purified by RP-FC (100 g C18 silica, 0-50% MeCN/water+0.1% TFA) to yield the title compound as a light brown solid (245 mg, 54%). LCMS: C₁₅H₁₇N₃O₃ requires: 287.1, found: m/z=288.2 [M+H]⁺.

### Example 79. 1-(4-(3-(piperazin-1-ylmethyl)pyrrolidin-1-yl)phenyl)dihydropyrimidine-2,4(1H,3H)-dione (HCB84)

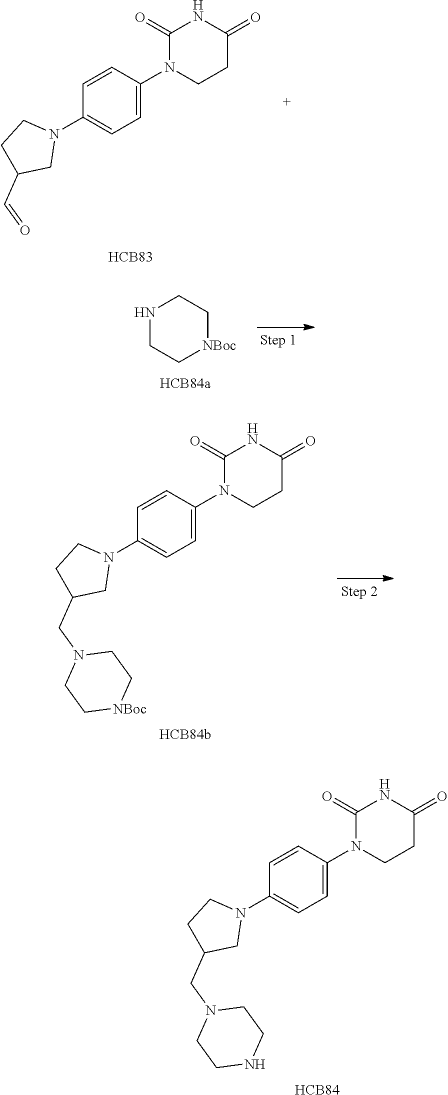 [SMILES: "HCB83" ; O=CC1CCN(c2ccc(N3CCC(=O)NC3=O)cc2)C1 ; CC(C)(C)OC(=O)N1CCN(CC2CCN(c3ccc(N4CCC(=O)NC4=O)cc3)C2)CC1 ; CC(C)(C)OC(=O)N1CCNCC1 ; "HCB84" ; O=C1CCN(c2ccc(N3CCC(CN4CCNCC4)C3)cc2)C(=O)N1]

### Step 1: Synthesis of tert-butyl 4-((1-(4-(2,4-dioxotetrahydropyrimidin-1(2H)-yl)phenyl)pyrrolidin-3-yl)methyl)piperazine-1-carboxylate

To a 2 dram vial was added rac-(3R)-1-[4-(2,4-dioxo-1,3-diazinan-1-yl)phenyl]pyrrolidine-3-carbaldehyde (30.00 mg, 0.10 mmol), tert-butyl piperazine-1-carboxylate (19.45 mg, 0.10 mmol), triethylamine (0.14 mL, 0.11 g, 1.04 mmol), DMSO (0.25 mL), and DCM (0.75 mL). To the reaction mixture was added sodium triacetoxyborohydride (66.39 mg, 0.31 mmol). The reaction mixture was stirred overnight, then diluted with sat. NaHCO₃(aq). The product was extracted with 10% MeOH/DCM (3×), dried over NaHCO₃, then concentrated. The resulting residue was purified by FC (4 g silica, 0-20% MeOH/DCM) to yield the title compound as an off-white solid (39.6 mg, 83%). LCMS: C₂₄H₃₅N₅O₄ requires: 457.3, found: m/z=458.4 [M+H]⁺.

### Step 2: Synthesis of 1-(4-(3-(piperazin-1-ylmethyl)pyrrolidin-1-yl)phenyl)dihydropyrimidine-2,4(1H,3H)-dione (HCB84)

To a 20 mL vial was added rac-tert-butyl 4-{[(3R)-1-[4-(2,4-dioxo-1,3-diazinan-1-yl)phenyl]pyrrolidin-3-yl]methyl}piperazine-1-carboxylate (39.60 mg, 0.09 mmol) and DCM (1.00 mL). To the reaction mixture was added 4N hydrogen chloride in dioxane (0.32 mL, 0.05 g, 1.30 mmol), resulting in the formation of a white precipitate. The reaction mixture was stirred for 1 h, then concentrated under reduced pressure to yield the title compound as a white solid (60 mg, quantitative yield). LCMS: C₁₉H₂₇N₅O₂ requires: 357.2, found: m/z=358.3 [M+H]⁺.

### Example 80. 3-{4-[4-(Piperazin-1-ylmethyl)piperidin-1-yl]phenyl}piperidine-2,6-dione (HCB85)

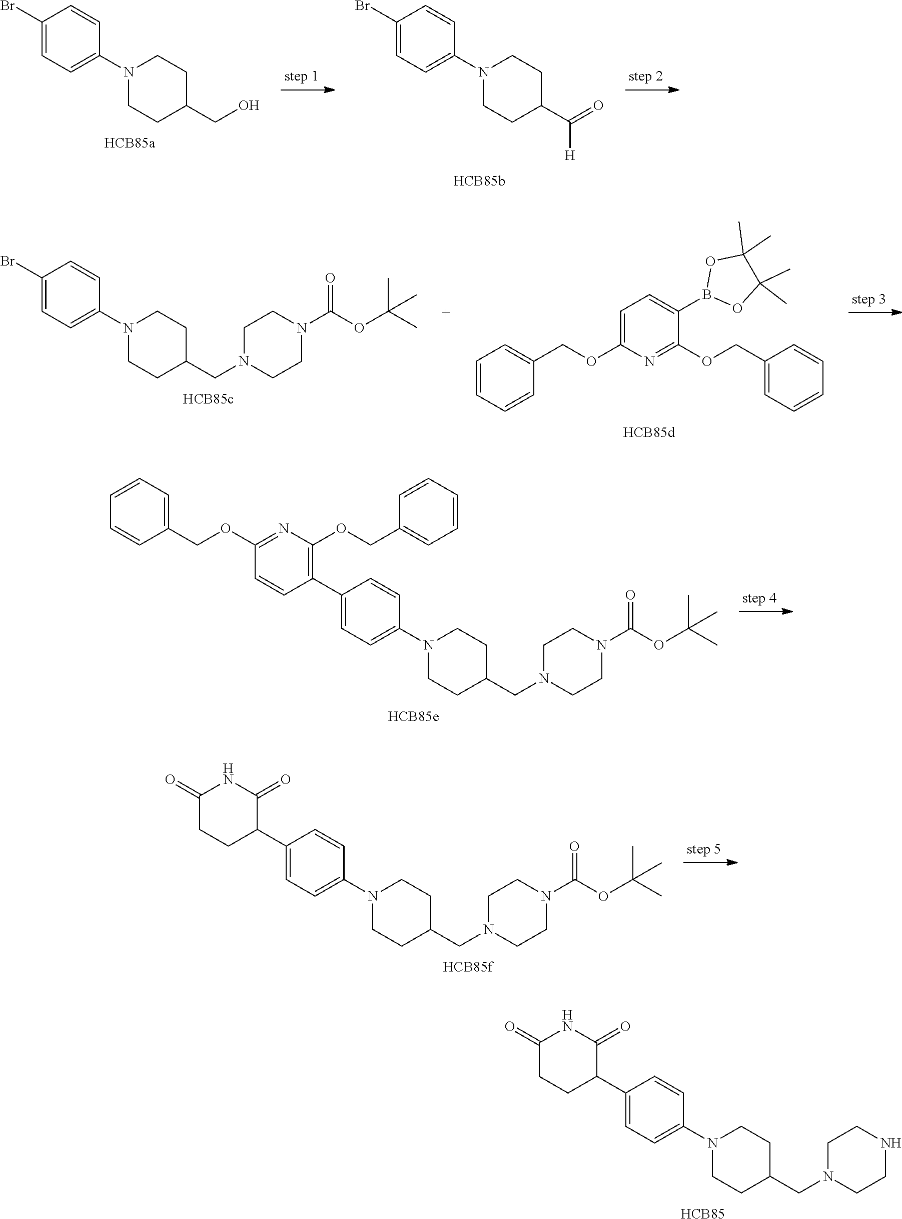

Step 1: To a solution of [1-(4-bromophenyl)piperidin-4-yl]methanol (6.70 g, 24.80 mmol) in 15 ml DMSO, triethylamine (69.71 mL, 50.19 g, 495.98 mmol) was added, cooled reaction mixture in ice-bath, sulfur trioxide pyridine complex (21.71 g, 136.40 mmol) powder was added slowly, the reaction mixture was stirred at room temperature for 1 hour. TLC indicated no SM left. Dissolved product in EtOAc, the organic layer was washed by water twice. The organic solution was dried over Na₂SO₄ and concentrated. The crude product was used to the next step without purification.

Step 2: To a solution of 1-(4-bromophenyl)piperidine-4-carbaldehyde (6.60 g, 24.61 mmol), triethylamine (3.43 mL, 2.49 g, 24.61 mmol) and tert-butyl piperazine-1-carboxylate (5.04 g, 27.07 mmol) in DCM, sodium triacetoxyborohydride (8.35 g, 39.38 mmol) was added in portion, the reaction mixture was stirred for 0.5 hour. The product was quenched with 10% NaHCO₃ solution, the solution was extracted by DCM twice, the organic solution was dried over Na₂SO₄ and concentrated. The crude product was purified by ISCO silica gel column eluted with 5-60% EtOAc in hexane to afford tert-butyl 4-{[1-(4-bromophenyl)piperidin-4-yl]methyl}piperazine-1-carboxylate (7.9 g, 73.2%) LCMS C₂₁H₃₂BrN₃O₂ required: 437.2 found: m/z=439.2 [M+H]⁺.

Step 3: tert-butyl 4-{[1-(4-bromophenyl)piperidin-4-yl]methyl}piperazine-1-carboxylate (2.40 g, 5.47 mmol), 2,6-bis(benzyloxy)-3-(4,4,5,5-tetramethyl-1,3,2-dioxaborolan-2-yl)pyridine (2.40 g, 5.75 mmol) and caesium carbonate (5.35 g, 16.42 mmol), Pd(dppf)C₁₂—CH₂Cl₂ (0.89 g, 1.09 mmol) were dissolved in 42 ml dioxane and 20 ml water, bubbled nitrogen gas. The reaction mixture was heated at 120° C. for 4 h hours. Dissolved product in EtOAc, washed with brine, the organic layer was dried over Na₂SO₄ and concentrated. The crude product was purified by ISCO silica gel column eluted with EtOAc/hexane to afford tert-butyl 4-[(1-{4-[2,6-bis(benzyloxy)pyridin-3-yl]phenyl}piperidin-4-yl)methyl]piperazine-1-carboxylate (2.33 g, 65.6%) LCMS C₄₀H₄₈N₄O₄ required: 648.4, found: m/z=649.5 [M+H]⁺.

Step 4: tert-butyl 4-[(1-{4-[2,6-bis(benzyloxy)pyridin-3-yl]phenyl}piperidin-4-yl)methyl]piperazine-1-carboxylate (2.33 g, 3.59 mmol) was in 250 ml round bottom flask, 10% wet Pd/C (1 g) was added, 40 ml EtOH and 20 ml THE were added, after purging, the solution was stirred under hydrogen balloon for 2 day. LCMS indicated reaction is completed. Filtered off Pd/C, washed the solid with DCM, the filtrate was evaporated under reduced pressure. The crude product was purified with ISCO silica gel column eluted with MeOH/DCM (0-6%), to afford tert-butyl 4-({1-[4-(2,6-dioxopiperidin-3-yl)phenyl]piperidin-4-yl}methyl)piperazine-1-carboxylate (1.17 g, 69.2%) LCMS C₂₆H₃₈N₄O₄ required: 470.3 found: m/z=471.5 [M+H]⁺.

Step 5: To a solution of tert-butyl 4-({1-[4-(2,6-dioxopiperidin-3-yl)phenyl]piperidin-4-yl}methyl)piperazine-1-carboxylate (0.4 g, 0.85 mmol) in 5 ml hexafluoro isopropanol, 1 ml TFA was added, the solution was left for 30 minutes. Evaporated all the solvent to afford 3-{4-[4-(piperazin-1-ylmethyl)piperidin-1-yl]phenyl}piperidine-2,6-dione as TFA salt. Quantitative yield.

### Example 81. 3-(2,4-dioxo-1,3-diazinan-1-yl)-4-fluorobenzoic acid (HCB86)

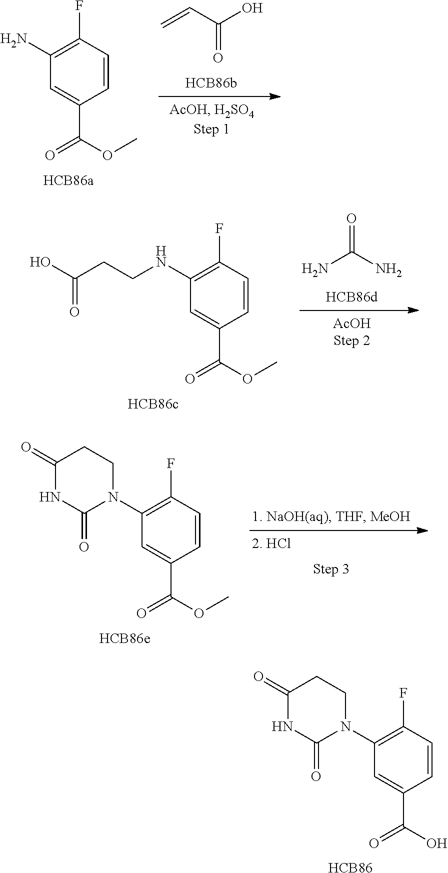

### Step 1: Synthesis of 3-{[2-fluoro-5-(methoxycarbonyl)phenyl]amino}propanoic acid

To a mixture of methyl 3-amino-4-fluorobenzoate (9 g, 53.206 mmol, 1 equiv) in AcOH (40 mL) was added acrylic acid (11.5 g, 159.618 mmol, 3 equiv) and H₂SO₄ (0.5 mL). The resulting mixture was stirred at 100° C. for 5 h. The resulting mixture was concentrated under vacuum. This resulted in 3-{[2-fluoro-5-(methoxycarbonyl)phenyl]amino}propanoic acid (13 g, crude). The crude product was used in the next step directly without further purification. MS (ESI) calc'd for (C₁₁H₁₂FNO₄) [M+1]⁺, 242.1; found, 242.1.

### Step 2: Synthesis of methyl 3-(2,4-dioxo-1,3-diazinan-1-yl)-4-fluorobenzoate

To a mixture of 3-[[2-fluoro-5-(methoxycarbonyl)phenyl]amino]propanoic acid (13 g, 53.893 mmol, 1 equiv) in AcOH (60 mL) was added urea (16.18 g, 269.465 mmol, 5 equiv). The resulting mixture was stirred at 120° C. overnight. The resulting mixture was concentrated under vacuum. The residue was purified by C18 reverse phase column with ACN/H₂O (1/1). This resulted in methyl 3-(2,4-dioxo-1,3-diazinan-1-yl)-4-fluorobenzoate as a white solid (3 g, 18.82%). MS (ESI) calc'd for (C₁₂H₁₁FN₂O₄) [M+1]⁺, 267.1; found, 267.1.

### Step 3: Synthesis of 3-(2,4-dioxo-1,3-diazinan-1-yl)-4-fluorobenzoic acid (HCB86)

To a mixture of methyl 3-(2,4-dioxo-1,3-diazinan-1-yl)-4-fluorobenzoate (3 g, 11.269 mmol, 1 equiv) in MeOH (20 mL) and THF (20 mL) were added NaOH (2.25 g, 56.254 mmol, 5 equiv) in H₂O (66 mL). The mixture was stirred at room temperature for 1 h. The resulting mixture was concentrated under vacuum. The residue was treated with 90 mL of 1M HCl. The resulting mixture was extracted with EtOAc, dried over anhydrous Na₂SO₄. After filtration, the filtrate was concentrated under reduced pressure. To the above mixture was added HCl (20 mL, 4N). The resulting mixture was stirred at 100° C. for 1 h, cooled and diluted with 75 mL of ice water. The precipitated solids were collected by filtration and washed with H₂O. This resulted in 3-(2,4-dioxo-1,3-diazinan-1-yl)-4-fluorobenzoic acid as an off-white solid (827.1 mg, 24.32%). MS (ESI) calc'd for (C₁₁H₉FN₂O₄) [M+1]⁺, 253.1; found, 253.2. ¹H NMR (400 MHz, DMSO-d₆) δ 13.18 (s, 1H), 10.54 (s, 1H), 8.06-7.99 (m, 1H), 7.98-7.90 (m, 1H), 7.49-7.40 (m, 1H), 3.81-3.73 (m, 2H), 2.78-2.71 (m, 2H)

### Example 82. 1-[4-(2,6-Dioxopiperidin-3-yl)phenyl]-4-fluoropiperidine-4-carbaldehyde (HCB87)

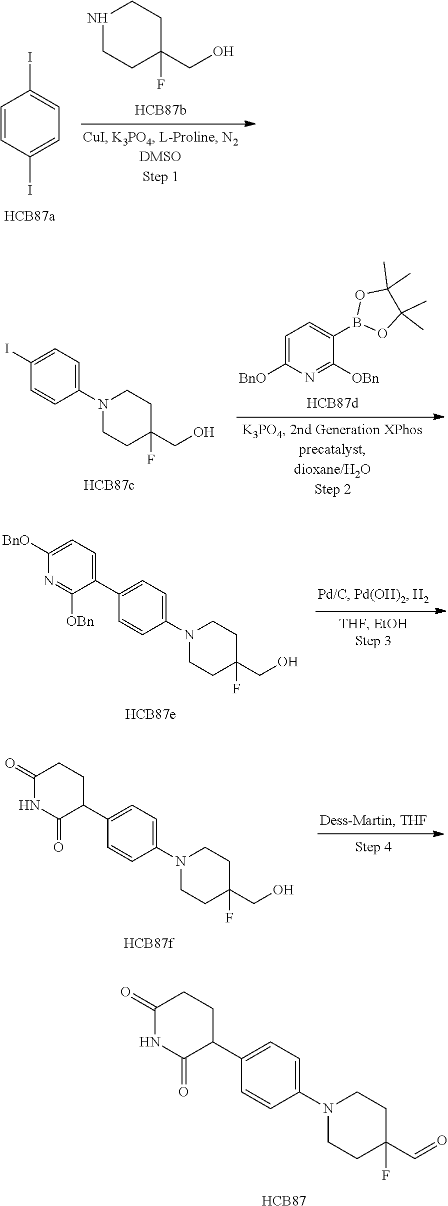

### Step 1: Synthesis of [4-fluoro-1-(4-iodophenyl)piperidin-4-yl]methanol

To a mixture of 1,4-diiodobenzene (1.5 g, 4.547 mmol, 1 equiv), (4-fluoropiperidin-4-yl)methanol (302.7 mg, 2.273 mmol, 0.5 equiv) in DMSO (20 mL) was added K₃PO₄ (2895.3 mg, 13.641 mmol, 3.0 equiv) and L-proline (0.104 g, 0.9118 mmol, 0.2 equiv) in portions at room temperature under nitrogen atmosphere. The resulting mixture was stirred for 2 h at 80° C. under nitrogen atmosphere. The resulting mixture was extracted with EtOAc. The combined organic layers were washed with water, dried over anhydrous Na₂SO₄. After filtration, the filtrate was concentrated under reduced pressure. The residue was purified by silica gel column chromatography, eluted with PE/EA (2/1) to afford [4-fluoro-1-(4-iodophenyl)piperidin-4-yl]methanol (800 mg, 52.50%) as a yellow solid. MS (ESI) calc'd for (C₁₂H₁₅FINO) [M+1]⁺, 336.1; found, 336.1 [M+H]⁺.

### Step 2: Synthesis of (1-{4-[2,6-bis(benzyloxy)pyridin-3-yl]phenyl}-4-fluoropiperidin-4-yl)methanol

To a mixture of [1-(4-bromophenyl)-4-fluoropiperidin-4-yl]methanol (2 g, 6.941 mmol, 1 equiv) and 2,6-bis(benzyloxy)-3-(4,4,5,5-tetramethyl-1,3,2-dioxaborolan-2-yl)pyridine (11.6 g, 27.764 mmol, 4 equiv) in dioxane (15 mL) and H₂O (3 mL) was added K₃PO₄ (4.4 g, 20.823 mmol, 3 equiv) and 2nd Generation XPhos precatalyst (1.1 g, 1.388 mmol, 0.2 equiv) at room temperature under nitrogen atmosphere. The reaction mixture was irradiated with microwave radiation for 2 h at 80° C. The resulting mixture was concentrated under vacuum. The residue was purified by silica gel column chromatography, eluted with CH₂Cl₂/EA (95/5) to afford (1-{4-[2,6-bis(benzyloxy)pyridin-3-yl]phenyl}-4-fluoropiperidin-4-yl)methanol (980 mg, 28.32%) as a yellow oil. MS (ESI) calc'd for (C₃₁H₃₁FN₂O₃) [M+1]⁺, 499.2; found, 499.2

### Step 3: Synthesis of 3-{4-[4-fluoro-4-(hydroxymethyl)piperidin-1-yl]phenyl}piperidine-2,6-dione

To a mixture of (1-{4-[2,6-bis(benzyloxy)pyridin-3-yl]phenyl}-4-fluoropiperidin-4-yl)methanol (950 mg, 1.905 mmol, 1 equiv) in THE (12 mL) was added Pd(OH)₂/C (475.3 mg) and Pd/C (474.5 mg) at room temperature. The resulting mixture was stirred for 3 h at room temperature under hydrogen atmosphere. The resulting mixture was filtered. The filtrate was concentrated under reduced pressure. This resulted in 3-{4-[4-fluoro-4-(hydroxymethyl)piperidin-1-yl]phenyl}piperidine-2,6-dione (480 mg, crude) as a white solid. MS (ESI) calc'd for (C₁₇H₂₁FN₂O₃)[M+1], 321.2; found, 321.1.

### Step 4: Synthesis of 1-[4-(2,6-dioxopiperidin-3-yl)phenyl]-4-fluoropiperidine-4-carbaldehyde (HCB87)

To a mixture of 3-{4-[4-fluoro-4-(hydroxymethyl)piperidin-1-yl]phenyl}piperidine-2,6-dione (300 mg, 0.936 mmol, 1 equiv) in THE (5 mL) was added Dess-Martin (794.4 mg, 1.872 mmol, 2 equiv) in portions at 0° C. under nitrogen atmosphere. The resulting mixture was stirred for 4 h at 0° C. under nitrogen atmosphere. The resulting mixture was concentrated under vacuum. The residue was purified by silica gel column chromatography, eluted with CH₂Cl₂/THE ( 88/12) to afford 1-[4-(2,6-dioxopiperidin-3-yl)phenyl]-4-fluoropiperidine-4-carbaldehyde (128.3 mg, 34.43%) as a white solid. MS (ESI) calc'd for (C₁₇H₁₉FN₂O₃)[M+1]⁺, 319.1; found, 319.1. ¹H NMR (400 MHz, DMSO-d₆) δ 10.77 (d, J=3.6 Hz, 1H), 9.71 (d, J=6.4 Hz, 1H), 7.08-7.03 (m, 2H), 6.97-6.91 (m, 2H), 6.00 (d, J=6.0 Hz, 1H), 3.76-3.72 (m 1H), 3.65-3.58 (m, 2H), 2.98-2.94 (m, 1H), 2.91-2.78 (m, 1H), 2.67-2.60 (m, 1H), 2.22-2.08 (m, 1H), 2.04-2.01 (m, 2H), 1.99-1.76 (m, 3H).

### Example 83. 3-(2-(4-(hydroxymethyl)piperidin-1-yl)pyrimidin-5-yl)piperidine-2,6-dione

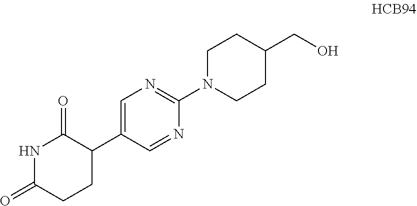 [SMILES: O=C1CCC(c2cnc(N3CCC(CO)CC3)nc2)C(=O)N1]

### Step 1: (1-(5-bromopyrimidin-2-yl)piperidin-4-yl)methanol

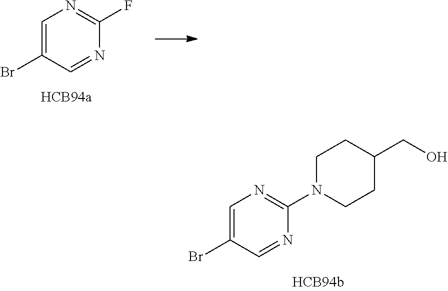 [SMILES: Fc1ncc(Br)cn1 ; OCC1CCN(c2ncc(Br)cn2)CC1]

A mixture of 5-bromo-2-fluoropyrimidine (1.15 g, 6.48 mmol), piperidin-4-ylmethanol (895 mg, 7.78 mmol), and potassium carbonate (2.69 g, 19.4 mmol) in DMSO (14.00 mL) was heated at 100° C. for 45 minutes. The mixture was transferred to a separatory funnel with ethylacetate and water. The organic layer was dried over Na₂SO₄ and concentrated. The crude residue was purified by flash chromatography on a 40 g column eluted with 0 to 100% ethylacetate/DCM to provide (1-(5-bromopyrimidin-2-yl)piperidin-4-yl)methanol (1.65 g, 93.4%). LCSM C₁₀H₁₄BrN₃O requires: 271, found: m/z=272 [M+H]⁺.

### Step 2: (1-(5-(2,6-bis(benzyloxy)pyridin-3-yl)pyrimidin-2-yl)piperidin-4-yl)methanol

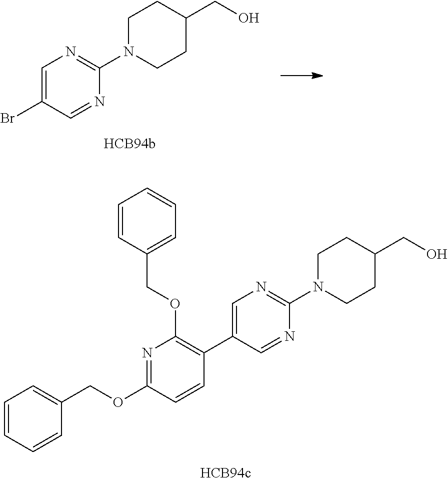 [SMILES: OCC1CCN(c2ncc(Br)cn2)CC1 ; OCC1CCN(c2ncc(-c3ccc(OCc4ccccc4)nc3OCc3ccccc3)cn2)CC1]

A mixture of (1-(5-bromopyrimidin-2-yl)piperidin-4-yl)methanol (510 mg, 1.87 mmol), 2,6-bis(benzyloxy)pyridin-3-ylboronic acid (628 mg, 1.87 mmol), tetrakis(triphenylphosphine)palladium(0) (217 mg, 0.19 mmol), and potassium carbonate (518 mg, 3.75 mmol) in THE (3.00 mL) and Water (1.00 mL) was microwaved at 120° C. for 40 minutes. The aq. layer was removed with a pipette. The organic layer was loaded directly onto a silica gel loading cartridge and purified by flash chromatography on a 40 g column eluted with 0 to 100% ethylacetate/DCM to provide (1-(5-(2,6-bis(benzyloxy)pyridin-3-yl)pyrimidin-2-yl)piperidin-4-yl)methanol (0.518 g, 57.3%). LCSM C₂₉H₃₀N₄03 requires: 482, found: m/z=483 [M+H]⁺.

### Step 3: 3-(2-(4-(hydroxymethyl)piperidin-1-yl)pyrimidin-5-yl)piperidine-2,6-dione

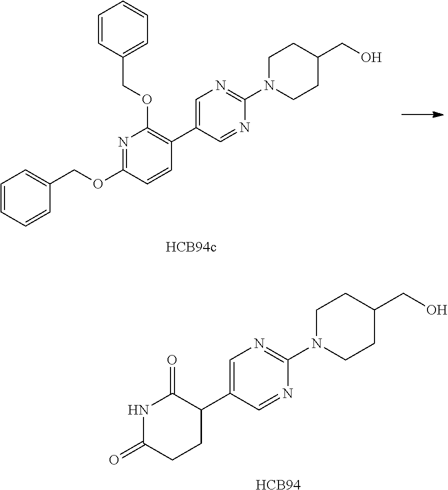 [SMILES: OCC1CCN(c2ncc(-c3ccc(OCc4ccccc4)nc3OCc3ccccc3)cn2)CC1 ; "HCB94" ; O=C1CCC(c2cnc(N3CCC(CO)CC3)nc2)C(=O)N1]

A mixture of (1-(5-(2,6-bis(benzyloxy)pyridin-3-yl)pyrimidin-2-yl)piperidin-4-yl)methanol (518 mg, 1.07 mmol) and 10% Pd/C (518 mg) in THE (5.00 mL) and EtOH (10.00 mL) was stirred under a balloon of H₂ for 2.5 hours. The mixture was diluted with 50 mL THE and filtered through a pad of celite. The resulting solution was concentrated in vacuo. The crude residue was purified by flash chromatography on a 40 g column eluted with 0 to 10% MeOH/DCM to provide 3-(2-(4-(hydroxymethyl)piperidin-1-yl)pyrimidin-5-yl)piperidine-2,6-dione (0.053 g, 16.2%). LCSM C₁₅H₂₀N₄O₃ requires: 304, found: m/z=305 [M+H]⁺.

### Step 4: Benzyl N-(4-fluoro-3-nitrophenyl)carbamate (Xsg)

Under a nitrogen atmosphere, sodium hydrogencarbonate (6.45 g, 76.86 mmol, 1.2 equiv) was dissolved in water (150 mL) and ethyl acetate (75 mL), and 4-fluoro-3-nitroaniline (10.0 g, 64 mmol, 1.0 equiv) was then added. Benzyl chloroformate (12.1 g, 70.45 mmol, 1.1 equiv) was then added dropwise at an internal temperature <30° C. The mixture was stirred at room temperature for 3 hr, and then left to stand until the layers separated. The organic phase was filtered, washed twice with 5 w/w % brine (150 mL), and concentrated under reduced pressure until the volume was about 20 mL. To this concentrated residue was added heptane (60 mL) at room temperature and the mixture was then stirred 30 min at room temperature. Additional heptane (250 mL) was added, and the mixture was stirred at room temperature for 2 hr. The precipitated crystals were collected by filtration and washed with a mixed solution of ethyl acetate (6 mL) and heptane (54 mL). The wet crystals were dried under reduced pressure to give benzyl 4-fluoro-3-nitrophenylcarbamate (16 g, yield 86%).

¹H NMR (300 MHz, DMSO-d₆) δ 10.28 (s, 1H), 8.34 (dd, J=6.8, 2.8 Hz, 1H), 7.76 (ddd, J=9.1, 3.9, 2.8 Hz, 1H), 7.54 (dd, J=11.2, 9.1 Hz, 1H), 7.48-7.32 (m, 5H), 5.19 (s, 2H).

### Step 5: Benzyl N-(4-fluoro-3-nitrophenyl)-N-methylcarbamate (X₈ h)

To a solution of benzyl 4-fluoro-3-nitrophenylcarbamate (16.0 g, 55.125 mmol, 1.0 equiv) in N,N-dimethylformamide (160 mL, 0.57 M) was added cesium carbonate (21.553 g, 66.15 mmol, 1.2 equiv). Methyl iodide (9.389 g, 66.148 mmol, 1.2 equiv) was then added dropwise at room temperature and the mixture was stirred at room temperature for 3 hr. Toluene (160 mL) and water (80 mL) were then added and this solution was stirred for about 10 min. The mixture was then left to stand until the layers separated. The organic layer was washed twice with water (100 mL), and concentrated under reduced pressure until the volume was about 50 mL. To this concentrated volume was added heptane (40 mL) and the mixture was stirred at room temperature for 30 min. Additional heptane (60 mL) was added, and the mixture was stirred for one hour at room temperature. The precipitated crystals were collected by filtration, and the obtained wet crystals were washed with a mixed solution of toluene (3 mL) and heptane (25 mL). The wet crystals were then dried under reduced pressure to give benzyl N-(4-fluoro-3-nitrophenyl)-N-methylcarbamate (16.0 g, 52.584 mmol, 95% yield). ¹H NMR (300 MHz, DMSO-d₆) δ 8.19 (dd, J=6.7, 2.8 Hz, 1H), 7.82 (ddd, J=9.0, 4.0, 2.8 Hz, 1H), 7.60 (dd, J=11.1, 9.0 Hz, 1H), 7.35 (d, J=3.8 Hz, 5H), 5.15 (s, 2H), 3.30 (s, 3H).

### Step 6: benzyl 2-(4-(((benzyloxy)carbonyl)(methyl)amino)-2-nitrophenyl)-3-((4aS,5aR)-5,5-difluoro-5a-methyl-1-(tetrahydro-2H-pyran-2-yl)-1,4,4a,5,5a,6-hexahydrocyclopropa[f]indazol-3-yl)-3-oxopropanoate (BBX₁₄)

To a solution of benzyl 3-[(4aS,5aR)-5,5-difluoro-5a-methyl-1-(oxan-2-yl)-1H,4H,4aH,5H,5aH,6H-cyclopropa[f]indazol-3-yl)-3-oxopropanoate (6.0 g, 13.499 mmol, 1.0 equiv) in anhydrous dimethyl sulfoxide (60.0 mL, 0.22 M) were successively added benzyl N-(4-fluoro-3-nitrophenyl)-N-methylcarbamate (4.929 g, 16.199 mmol, 1.2 equiv) and potassium carbonate (3.731 g, 26.996 mmol, 2.0 equiv). The mixture was stirred in an oil bath at 90° C. for 2 hr and 20 min. The reaction mixture was then cooled to room temperature, poured into 5% aq. citric acid solution (100 mL), and extracted with ethyl acetate. The organic layer was washed with water, brine, dried over anhydrous sodium sulfate, and concentrated under reduced pressure. The obtained residue was purified by silica gel flash chromatography (ethyl acetate:hexane 2:1), to give the title product as a light yellow foamy solid (8.24 g, 84% yield). ¹H NMR (300 MHz, DMSO-d₆) δ 8.21-8.01 (m, 1H), 7.74 (ddt, J=9.4, 6.4, 2.8 Hz, 1H), 7.53-7.13 (m, 10H), 6.37-6.22 (m, 1H), 5.44 (dd, J=16.9, 8.0 Hz, 1H), 5.31-5.08 (m, 4H), 3.74 (s, 1H), 3.68-3.48 (m, 2H), 3.23-3.12 (m, 1H), 3.00 (s, 3H), 2.76 (s, 1H), 2.03 (s, 1H), 1.78 (t, J=15.4 Hz, 3H), 1.51 (s, 3H), 1.34 (s, 3H). LCMS: C₃₉H₃₈F₂N₄O₈ requires: 728, found: m/z=729 [M+H]⁺.

### Example 100. 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-N-methyl-1H-indol-6-amine (BBX₁₅)

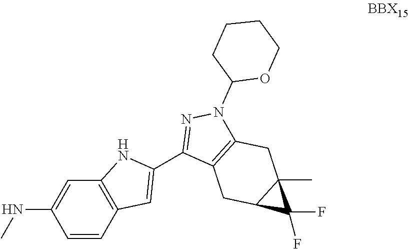 [SMILES: CNc1ccc2cc(-c3nn(C4CCCCO4)c4c3C[C@@H]3C(F)(F)[C@]3(C)C4)[nH]c2c1]

Synthesis of BBX₁₅ is shown in Step 2 of Example 73 below.

### Example 101. (S)—N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperazin-1-yl)propenamide (BBX₁₆)

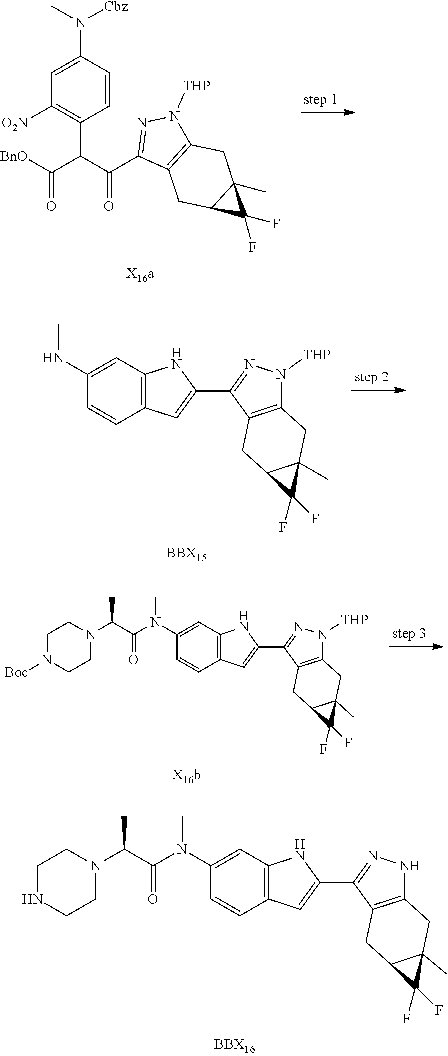

### Step 1: 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-N-methyl-1H-indol-6-amine (BBX₁₅)

A mixture of benzyl N-{2-[(4aS,5aR)-5,5-difluoro-5a-methyl-1-(oxan-2-yl)-4H,4aH,6H-cyclopropa[f]indazol-3-yl)-1H-indol-6-yl}-N-methylcarbamate (46 mg, 0.08 mmol) and 10% Pd/C (5 mg) in EtOH (1 mL) and toluene (1 mL) was stirred under a balloon of H₂. After three hours, 10% Pd/C (5 mg) was added. After four hours the mixture was diluted with THF, filtered through celite, and toluene (5 mL) was added. The mixture was concentrated to about one milliliter and toluene (5 mL) was added. The mixture was then concentrated to about one milliliter and taken into the next step without further purification as a solution of 2-[(4aS,5aR)-5,5-difluoro-5a-methyl-1-(oxan-2-yl)-4H,4aH,6H-cyclopropa[f]indazol-3-yl]-N-methyl-1H-indol-6-amine in toluene. LCMS: C₂₃H₂₆F₂N₄O requires: 412, found: m/z=413 [M+H]⁺.

### Step 2: tert-butyl 4-((2S)-1-((2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)(methyl)amino)-1-oxopropan-2-yl)piperazine-1-carboxylate (X₁₆b)

To a mixture of (2S)-2-[4-(tert-butoxycarbonyl)piperazin-1-yl]propanoic acid (21 mg, 0.08 mmol) in DMF (1 mL) was added HATU (32 mg, 0.08 mmol) and N,N-diisopropylethylamine (0.06 mL, 0.34 mmol). After five minutes, the mixture was added to a solution of 2-[(4aS,5aR)-5,5-difluoro-5a-methyl-1-(oxan-2-yl)-4H,4aH,6H-cyclopropa[ ]indazol-3-yl]-N-methyl-1H-indol-6-amine (35 mg, 0.08 mmol) in toluene (1 mL). After stirring overnight, the mixture was transferred to a separatory funnel containing ethyl acetate. The resulting solution was washed with water, and brine was added to get the layers to separate. The organic layer was dried over Na₂SO₄ and concentrated. The crude residue was purified by flash chromatography on a 12 g column eluted with 0 to 5% MeOH:DCM to provide tert-butyl 4-[(1S)-1-({2-[(4aS,5aR)-5,5-difluoro-5a-methyl-1-(oxan-2-yl)-4H,4aH,6H-cyclopropa[indazol-3-yl]-1H-indol-6-yl}(methyl)carbamoyl)ethyl]piperazine-1-carboxylate (0.052 g, quant.). LCMS: C₃₅H₄₆F₂N₆O₄ requires: 652, found: m/z=653 [M+H]⁺.

### Step 3: (S)—N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperazin-1-yl)propanamide (BBX₁₆)

Following the procedure described at Step 6, in the above procedure for BBX₉, and starting from tert-butyl 4-((2S)-1-((2-((4aS,5aR)-5,5-difluoro-5a-methyl-1-(tetrahydro-2H-pyran-2-yl)-1,4,4a,5,5a,6-hexahydrocyclopropa[ ]indazol-3-yl)-1H-indol-6-yl)(methyl)amino)-1-oxopropan-2-yl)piperazine-1-carboxylate (600 mg, 0.919 mmol) provided (S)—N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperazin-1-yl)propanamide (150 mg, 0.321 mmol, 35%).

### Example 102. N-(2-((4aS,5aR)-5,5-difluoro-5a-methyl-1,4,4a,5,5a,6-hexahydrocyclopropa[f]indazol-3-yl)-1H-indol-6-yl)-N-methyl-2-(piperidin-4-yl)acetamide (BBX₁₇)

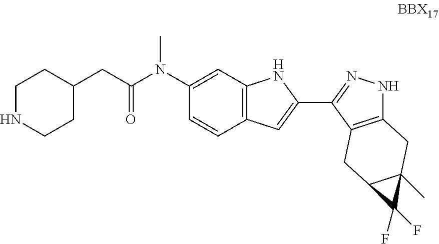 [SMILES: CN(C(=O)CC1CCNCC1)c1ccc2cc(-c3n[nH]c4c3C[C@@H]3C(F)(F)[C@]3(C)C4)[nH]c2c1]

Following the procedure for BBX₁₆ and starting from [1-(tert-butoxycarbonyl)piperidin-4-yl]acetic acid (50 mg, 0.21 mmol) provided N-{2-[(4aS,5aR)-5,5-difluoro-5a-methyl-1H,4H,4aH,6H-cyclopropa[indazol-3-yl]-1H-indol-6-yl}-N-methyl-2-(piperidin-4-yl)acetamide (BBX₁₇) (0.082 g, quant.). LCMS: C₂₅H₂₉F₂N₅O requires: 453.2, found: m/z=454.4 [M+H]⁺.

### Example 103. N-{2-[(4aS,5aR)-5,5-difluoro-5a-methyl-1H,4H,4aH,6H-cyclopropa[f]indazol-3-yl]-1H-indol-6-yl}-N-methylpiperidine-4-carboxamide (BBX₁₈)

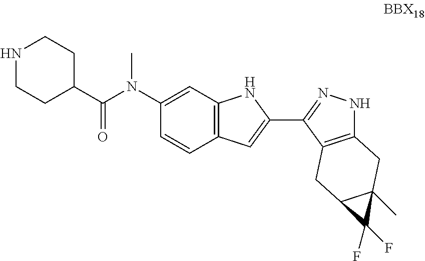 [SMILES: CN(C(=O)C1CCNCC1)c1ccc2cc(-c3n[nH]c4c3C[C@@H]3C(F)(F)[C@]3(C)C4)[nH]c2c1]

Following the procedure for BBX₁₄ and starting from 1-(tert-butoxycarbonyl)piperidine-4-carboxylic acid (72 mg, 0.31 mmol) provided N-{2-[(4aS,5aR)-5,5-difluoro-5a-methyl-1H,4H,4aH,6H-cyclopropa[indazol-3-yl]-1H-indol-6-yl}-N-methylpiperidine-4-carboxamide (0.072 g, quant.). LCMS: C₂₄H₂₇F₂N₅O requires: 439.2, found: m/z=440.4 [M+H]⁺.

### Example 104. 2-(6,6-dimethyl-4,5,6,7-tetrahydro-1H-indazol-3-yl)-1H-indole-6-carboxylic acid (BBX₁₉)

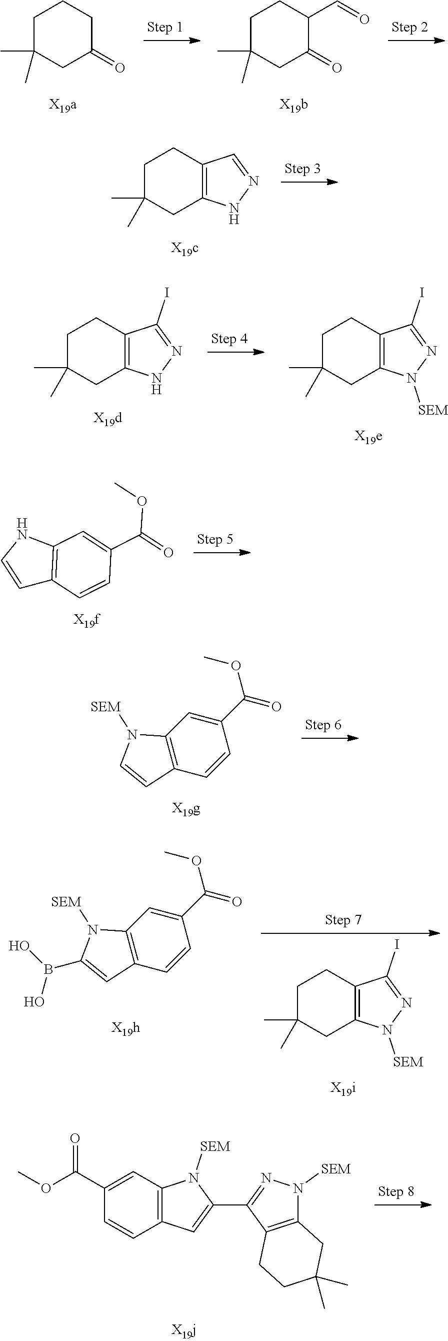

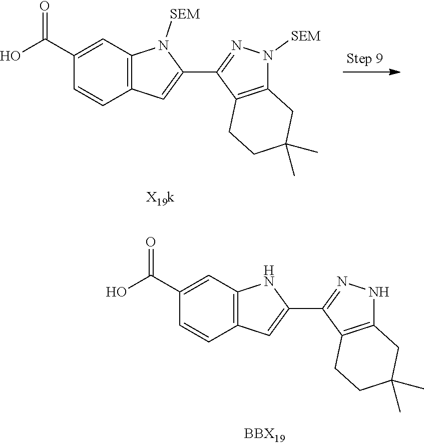 [SMILES: CC1(C)CCc2c(-c3cc4ccc(C(=O)O)cc4n3COCC[Si](C)(C)C)nn(COCC[Si](C)(C)C)c2C1 ; CC1(C)CCc2c(-c3cc4ccc(C(=O)O)cc4[nH]3)n[nH]c2C1]

### Step 1: 4,4-Dimethyl-2-oxocyclohexanecarboxaldehyde (X₁₉b)

To a suspension of NaH (17.431 g, 436 mmol, 1.1 equiv) in THF (310 mL), under nitrogen atmosphere, was added a solution of 3,3-dimethylcyclohexanone (50.0 g, 396 mmol, 1.0 equiv) in THE (160 mL) dropwise at 0° C. over about one hour, and the mixture was stirred for one hour. Then, a solution of ethyl formate (60 g, 812 mmol, 2.05 equiv) in THE (160 mL) was added dropwise at 0° C. for one hour and stirred for another two hours from 0° C. to room temperature. At completion, to the reaction mixture was added water and diethyl ether, and the organic layer was separated and extracted with 2 N NaOH solution. The aqueous layer was acidified with concentrated HCl and extracted with diethyl ether. Then, the organic layer was washed with brine, dried over sodium sulfate, filtered, and concentrated under reduced pressure to afford the title compound (50 g, 82% yield). ¹H NMR (300 MHz, DMSO-d₆) δ 11.42 (s, 1H), 10.01-8.98 (m, 1H), 2.19 (d, J=7.7 Hz, 2H), 2.06 (s, 2H), 1.37 (t, J=6.7 Hz, 2H), 0.91 (s, 6H).

### Step 2: 6,6-dimethyl-4,5,6,7-tetrahydro-1H-indazole (X₁₉c)

To a solution of 4,4-dimethyl-2-oxocyclohexanecarboxaldehyde (50.0 g, 324 mmol, 1.0 equiv) in MeOH (235 mL) was added a solution of hydrazine hydrate (25 mL, 327 mmol, 1.01 equiv) in MeOH (20 mL) dropwise with heating under reflux over one hour and the mixture was then stirred for 15 min. The reaction mixture was concentrated under reduced pressure, ethyl acetate and water were added, and the organic layer was separated. Then the organic layer was washed with brine, dried over sodium sulfate, filtered, and concentrated under reduced pressure to afford the title compound (46.6 g, 96% yield). ¹H NMR (300 MHz, DMSO-d₆) δ 12.20 (s, 1H), 7.25 (s, 1H), 2.61-2.35 (m, 4H), 1.44 (t, J=6.5 Hz, 2H), 1.18 (t, J=7.1 Hz, 1H), 0.94 (s, 6H).

### Step 3: 3-iodo-6,6-dimethyl-4,5,6,7-tetrahydro-1H-indazole (X₁₉a)

To a solution of 6,6-dimethyl-4,5,6,7-tetrahydro-1H-indazole (50 g, 333 mmol, 1.0 equiv) in DMF (950 mL, 0.35 M) was added iodine (212 g, 835 mmol, 2.5 equiv) and KOH (92 g, 1647 mmol, 4.95 equiv) at rt and the mixture was stirred for 4 h. At completion, the reaction was cooled in an ice bath and an aqueous solution of sodium hydrogensulfite (25 g in 250 mL water) was added dropwise. More water (625 mL) was then added. A precipitate appeared and was filtered to obtain the title compound (56 g, 65% yield). ¹H NMR (300 MHz, DMSO-d₆) δ 12.69 (s, 1H), 2.33 (s, 2H), 2.21 (t, J=6.4 Hz, 2H), 1.47 (t, J=6.4 Hz, 2H), 0.94 (s, 6H).

### Step 4: 3-iodo-6,6-dimethyl-1-{[2-(trimethylsilyl)ethoxy]methyl}-4,5,6,7-tetrahydro-1H-indazole (X₁₉e)

To a solution of 3-iodo-6,6-dimethyl-4,5,6,7-tetrahydro-1H-indazole (56 g, 203 mmol, 1.0 equiv) in DMF (450 mL, 0.45 M), under an argon atmosphere, was added NaH (8.9 g, 223 mmol, 1.1 equiv) in 500 mg portions at an internal temperature of −10° C. The mixture was then stirred for 30 min. Then, (2-(Chloromethoxy)ethyl)trimethylsilane (35.9 mL, 203 mmol, 1.0 equiv) was added dropwise over 15 min, and the mixture was further stirred for 2 h. At completion, ethyl acetate and water were added, the organic layer was separated, washed with ice-cold water and brine, dried over sodium sulfate, filtered, and concentrated under reduced pressure to afford the title compound (71.5 g, 97% yield). ¹H NMR (300 MHz, DMSO-d₆) δ 5.30 (d, J=17.8 Hz, 2H), 3.49 (dt, J=15.7, 7.9 Hz, 2H), 2.40 (s, 1H), 2.31 (s, 1H), 2.30-2.16 (m, 2H), 1.48 (q, J=6.2 Hz, 2H), 0.94 (d, J=6.5 Hz, 6H), 0.79 (t, J=7.9 Hz, 2H), −0.06 (d, J=1.2 Hz, 9H).

### Step 5: methyl 1-{[2-(trimethylsilyl)ethoxy]methyl}-1H-indole-6-carboxylate (X_{19g})

To a solution of methyl 1H-indole-6-carboxylate (60 g, 342.5 mmol, 1.0 equiv) in DMF (685 mL, 0.5 M), under an argon atmosphere, was added NaH (14.5 g, 376.8 mmol, 1.1 equiv) in portions at an internal temperature of −10° C., and the mixture was then stirred for one hour. Then, (2-(Chloromethoxy)ethyl)trimethylsilane (60.6 ml, 342.5 mmol, 1.0 equiv) was added dropwise over 1 h, and the mixture was further stirred overnight. At completion, ethyl acetate and water were added to the reaction mixture, and the organic layer was separated. Then, the organic layer was washed with brine, dried over sodium sulfate, filtered, and concentrated under reduced pressure. The residue was used in the next step without purification. ¹H NMR (300 MHz, DMSO-d₆) δ 8.21 (s, 1H), 7.76-7.65 (m, 3H), 6.61-6.55 (m, 1H), 5.64 (s, 2H), 3.87 (s, 3H), 3.50-3.38 (m, 2H), 0.87-0.74 (m, 2H), −0.11 (s, 9H).

### Step 6: [6-(methoxycarbonyl)-1-{[2-(trimethylsilyl)ethoxy]methyl}-1H-indol-2-yl]boronic acid (X₁₉ h)

To a solution of methyl 1-{[2-(trimethylsilyl)ethoxy]methyl}-1H-indole-6-carboxylate (20 g, 65.5 mmol, 1.0 equiv) in THE (66 mL, 1 M) was added triisopropylborate (23 mL, 19 mmol, 1.5 equiv), and the reaction mixture was then cooled to −10° C. Then, LDA (2 M solution in hexane, 44.2 mL, 88.4 mmol, 1.35 equiv) was added dropwise over 20 min while maintaining the internal temperature at −10° C. The mixture was further stirred for one hour. Then, the reaction mixture was cooled in an ice bath and 10% aqueous NaHCO₃ solution was added dropwise until pH 9. The aqueous layer was extracted three times with ethyl acetate, and the combined organic layers were washed with brine, dried over magnesium sulfate, filtered, and the filtrate was concentrated under reduced pressure. The crude material was used in the next step without further purification.

### Step 7: methyl 2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carboxylate (X₁₉i)

Under an argon atmosphere, 3-iodo-6,6-dimethyl-1-{[2-(trimethylsilyl)ethoxy]methyl}-4,5,6,7-tetrahydro-1H-indazole (36.7 g, 90.4 mmol, 1.0 equiv), potassium phosphate tribasic (76.74 g, 361.5 mmol, 4 equiv), and Pd(dppf)Cl₂·DCM complex (7.42 g, 9.04 mmol, 0.1 equiv) were mixed in a mixture of dioxane (532 mL) and water (226 mL). Then, [6-(methoxycarbonyl)-1-{[2-(trimethylsilyl)ethoxy]methyl}-1H-indol-2-yl]boronic acid (38 g, 108.5 mmol, 1.2 equiv) was added in portions with heating at 110° C. over 15 min, and the mixture was stirred for another 5 min. After cooling, water and ethyl acetate were added to the reaction mixture, and the organic layer was separated. Then, the organic layer was washed with water and saturated brine, dried over sodium sulfate, filtered, and concentrated under reduced pressure. The residue was purified by flash chromatography (5% to 15% ethyl acetate in hexane) to give the title compound (28 g, 48% yield over three steps). ¹H NMR (300 MHz, DMSO-d₆) δ 8.22 (s, 1H), 7.74-7.64 (m, 2H), 6.83 (s, 1H), 6.14 (s, 2H), 5.42 (s, 2H), 3.88 (s, 3H), 3.66-3.52 (m, 4H), 2.68-2.56 (m, 2H), 1.54 (t, J=5.9 Hz, 2H), 1.18 (t, J=7.1 Hz, 2H), 1.03 (s, 6H), 0.92-0.83 (m, 2H), 0.73-0.63 (m, 2H), −0.03 (s, 9H), −0.21 (s, 9H).

### Step 8: 2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carboxylic acid (X₁₉j)

To a solution of methyl 2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carboxylate (16 g, 27.4 mmol, 1.0 equiv) in a mixture of THF (80 mL) and MeOH (80 mL) was added 4N NaOH (34.2 mL, 137 mmol, 5 equiv), and the mixture was heated at 60° C. for 2 h. After cooling, the reaction mixture was concentrated, and 10% aqueous citric acid solution was added to the residue to adjust the pH to five. The mixture was extracted with ethyl acetate, washed with brine, dried over sodium sulfate, filtered, and concentrated under reduced pressure. The compound was purified via flash chromatography (hexane:ethyl acetate 8:2) to give the title compound (13.4 g, 85% yield). ¹H NMR (300 MHz, DMSO-d₆) δ 12.63 (s, 1H), 8.22 (s, 1H), 7.74-7.64 (m, 2H), 6.83 (s, 1H), 6.14 (s, 2H), 5.42 (s, 2H), 3.66-3.52 (m, 4H), 2.68-2.56 (m, 2H), 1.54 (t, J=5.9 Hz, 2H), 1.18 (t, J=7.1 Hz, 2H), 1.03 (s, 6H), 0.92-0.83 (m, 2H), 0.73-0.63 (m, 2H), −0.03 (s, 9H), −0.21 (s, 9H). LCMS: C₃₀H₄₇N₃O₄Si₂ requires: 569, found: m/z=570 [M+H]⁺.

### Step 9: 2-(6,6-dimethyl-4,5,6,7-tetrahydro-1H-indazol-3-yl)-1H-indole-6-carboxylic acid (BBX₁₉)

Bu₄NF (1.0 M in THF, 2.63 mL, 2.63 mmol) and ethylenediamine (528 μL, 7.9 mmol) was added to a solution of 2-(6,6-dimethyl-1-{[2-(trimethylsilyl)ethoxy]methyl}-5,7-dihydro-4H-indazol-3-yl)-1-{[2-(trimethylsilyl)ethoxy]methyl}indole-6-carboxylic acid (300 mg, 0.53 mmol) in DME (2.65 mL). The reaction mixture was stirred at 90° C. for 16 h, and then was concentrated under reduced pressure. The residue was dissolved in EtOAc, and acidified with 10% aqueous citric acid (pH −4-5). The aqueous layer was extracted with EtOAc. The combined organic layer was dried over Na₂SO₄, and concentrated under reduced pressure. Flash chromatography (SiO₂, 0-100% H₂O:MeCN gradient elution) afforded the desired product (87.4 mg, 0.28 mmol, 54%). LCMS: C₁₈H₁₉N₃O₂ requires: 309, found: m/z=310 [M+H]⁺.

### Example 105. (2-(6,6-dimethyl-4,5,6,7-tetrahydro-1H-indazol-3-yl)-1H-indol-6-yl)(piperazin-1-yl)methanone (BBX₂₀)

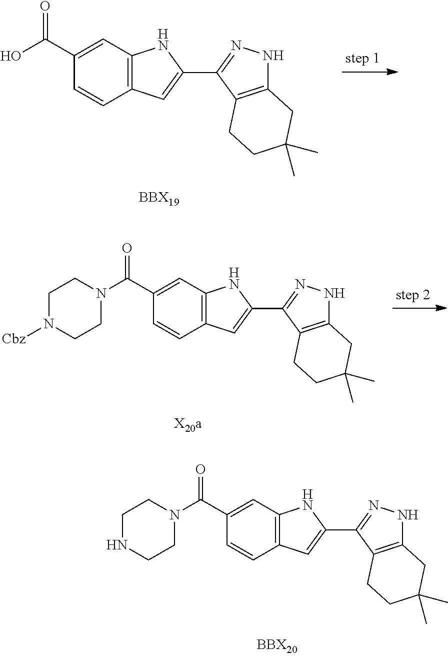 [SMILES: CC1(C)CCc2c(-c3cc4ccc(C(=O)O)cc4[nH]3)n[nH]c2C1 ; CC1(C)CCc2c(-c3cc4ccc(C(=O)N5CCN(C(=O)OCc6ccccc6)CC5)cc4[nH]3)n[nH]c2C1 ; CC1(C)CCc2c(-c3cc4ccc(C(=O)N5CCNCC5)cc4[nH]3)n[nH]c2C1]

### Step 1: Benzyl 4-[2-(6,6-dimethyl-4,5,6,7-tetrahydro-1H-indazol-3-yl)-1H-indole-6-carbonyl]piperazine-1-carboxylate (X₂₀ₐ)

To a solution of 2-(6,6-dimethyl-4,5,6,7-tetrahydro-1H-indazol-3-yl)-1H-indole-6-carboxylic acid (1 g, 3.235 mmol), i-Pr₂NEt (1.69 mL, 9.702 mmol), benzyl 1-piperazinecarboxylate (0.624 ml, 3.235 mmol) in DMF (16 mL) was added HATU (1.845 g, 4.852 mmol) in DMF (5 mL) at 0° C. After stirred at room temperature for 1 hour, the reaction mixture was poured onto crushed ice and extracted with EtOAc. The combined organic layers were dried over Na₂SO₄, and concentrated under reduced pressure. Flash chromatography using EtOAc:hexanes (4:6) afforded the desired product (0.92 g, 1.8 mmol, 56%). ¹H NMR (300 MHz, (CD₃)₂SO) δ 12.58 (s, 1H), 11.48 (s, 1H), 7.54 (d, J=8.1 Hz, 1H), 7.47 (s, 1H), 7.41-7.28 (m, 5H), 7.03 (d, J=7.8 Hz, 1H), 6.62 (s, 1H), 5.11 (s, 2H), 3.51 (d, J=20.5 Hz, 8H), 2.68 (d, J=4.5 Hz, 2H), 2.42 (s, 2H), 1.58 (t, J=6.2 Hz, 2H), 1.01 (s, 6H). LCMS: C₃₀H₃₃N₅O₃ requires: 511, found: m/z=512 [M+H]⁺.

### Step 2: (2-(6,6-dimethyl-4,5,6,7-tetrahydro-1H-indazol-3-yl)-1H-indol-6-yl)(piperazin-1-yl)methanone (BBX₂₀)

A solution of benzyl 4-[2-(6,6-dimethyl-1,4,5,7-tetrahydroindazol-3-yl)-1H-indole-6-carbonyl]piperazine-1-carboxylate (250 mg, 0.49 mmol) in MeOH (5 mL) and DCM (5 mL) was stirred under H₂ (1 atm) in the presence of Pd/C (50 mg) for one hour. The reaction mixture was filtered through Celite, and concentrated under reduced pressure to afford the desired product (104 mg, 0.27 mmol, 55%). LCMS: C₂₂H₂₇N₅O requires: 377, found: m/z=378 [M+H]⁺.

### Example 106. (2-(6,6-dimethyl-4,5,6,7-tetrahydro-1H-indazol-3-yl)-1H-indol-6-yl)(piperazin-1-yl)methanone (BBX₂₁)

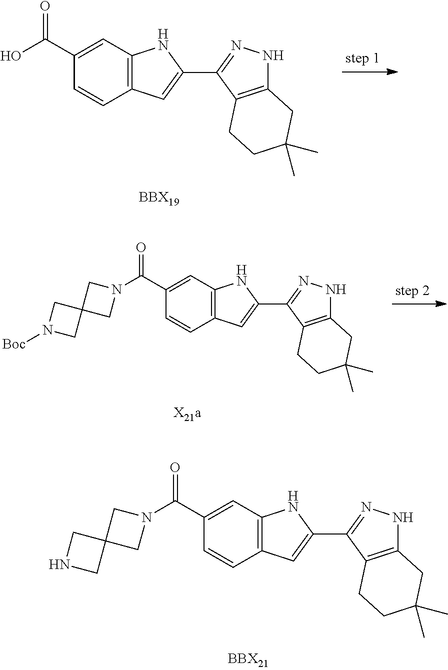 [SMILES: CC1(C)CCc2c(-c3cc4ccc(C(=O)O)cc4[nH]3)n[nH]c2C1 ; CC1(C)CCc2c(-c3cc4ccc(C(=O)N5CC6(CN(C(=O)OC(C)(C)C)C6)C5)cc4[nH]3)n[nH]c2C1 ; CC1(C)CCc2c(-c3cc4ccc(C(=O)N5CC6(CNC6)C5)cc4[nH]3)n[nH]c2C1]

### Step 1: tert-butyl 6-[2-(6,6-dimethyl-1,4,5,7-tetrahydroindazol-3-yl)-1H-indole-6-carbonyl]-2,6-diazaspiro[3.3]heptane-2-carboxylate (X₂₁a)

BOP (61.9 mg, 0.14 mmol) was added to a mixture of 2-(6,6-dimethyl-1,4,5,7-tetrahydroindazol-3-yl)-1H-indole-6-carboxylic acid (43.3 mg, 0.14 mmol), tert-butyl 2,6-diazaspiro[3.3]heptane-2-carboxylate (27.8 mg, 0.14 mmol), andN,N-diisopropylethylamine (0.07 mL, 0.05 g, 0.42 mmol) in DMF (0.70 mL). After stirring overnight, water (1 mL) was added. The mixture was extracted twice with DCM (1 mL). The combined organic layers were concentrated to provide tert-butyl 6-[2-(6,6-dimethyl-1,4,5,7-tetrahydroindazol-3-yl)-1H-indole-6-carbonyl]-2,6-diazaspiro[3.3]heptane-2-carboxylate (68 mg, 100%). LCMS: C₂₈H₃₅N₅O₃ requires: 490, found: m/z=318 [M+H]⁺ 491.

### Step 2: 3-(6-{2,6-diazaspiro[3.3]heptane-2-carbonyl}-1H-indol-2-yl)-6,6-dimethyl-1,4,5,7-tetrahydroindazole (BBX₂₁)

tert-butyl 6-[2-(6,6-dimethyl-1,4,5,7-tetrahydroindazol-3-yl)-1H-indole-6-carbonyl]-2,6-diazaspiro[3.3]heptane-2-carboxylate (68.6 mg, 0.14 mmol) was stirred in DCM (0.50 mL) and TFA (0.50 mL) for fifteen minutes. The mixture was then concentrated to provide 3-(6-{2,6-diazaspiro[3.3]heptane-2-carbonyl}-1H-indol-2-yl)-6,6-dimethyl-1,4,5,7-tetrahydroindazole (52 mg, 0.14 mmol). LCMS: C₂₃H₂₇N₅O requires 389, found: m/z=390 [M+H]⁺.

### General Procedure H1: Amidation

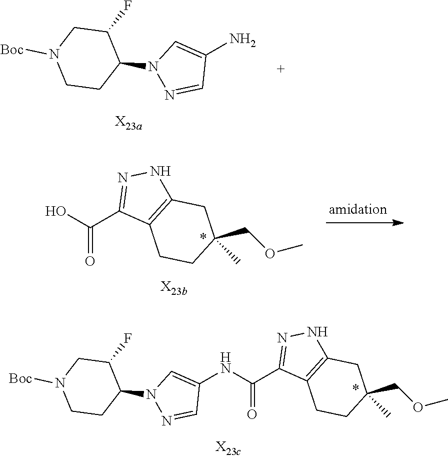 [SMILES: CC(C)(C)OC(=O)N1CC[C@H](n2cc(N)cn2)[C@@H](F)C1 ; COC[C@@]1(C)CCc2c(C(=O)O)n[nH]c2C1 ; COC[C@@]1(C)CCc2c(C(=O)Nc3cnn([C@H]4CCN(C(=O)OC(C)(C)C)C[C@@H]4F)c3)n[nH]c2C1]

Scheme H₁: Synthesis of BBX₂₃ Via Amide Formation

A mixture of amine X²³a (45 mg, 0.16 mmol), acid X²³n (30 mg, 0.14 mmol), HATU (54 mg, 0.14 mmol), i-Pr₂NEt (75 μL, 0.43 mmol) in DMF (500 μL) was allowed to stir at rt for 16 hr. The reaction was quenched with H₂O. The reaction mixture was extracted with EtOAc, washed with saturated aqueous NaCl, dried over Na₂SO₄, and concentrated under reduced pressure. Flash chromatography (SiO₂, 0 to 80% EtOAc/hexanes gradient elution) provided the amide product X²³c (57 mg, 0.12 mmol, 81%). LCMS: C₂₄H₃₅FN₆O₄ requires: 490, found: m/z=491 [M+H]⁺. An exemplary amide coupling is provided in the Scheme H₁ below where tert-butyl (3S,4S)-4-(4-amino-1H-pyrazol-1-yl)-3-fluoropiperidine-1-carboxylate and (S)-6-(methoxymethyl)-6-methyl-4,5,6,7-tetrahydro-1H-indazole-3-carboxylic acid were reacted as described above to provide tert-butyl (3S,4S)-3-fluoro-4-(4-((S)-6-(methoxymethyl)-6-methyl-4,5,6,7-tetrahydro-1H-indazole-3-carboxamido)-1H-pyrazol-1-yl)piperidine-1-carboxylate (X_{23c}, *arbitrarily assigned).

### Example 107. tert-butyl (3S,4S)-3-fluoro-4-(4-((R)-6-(methoxymethyl)-6-methyl-4,5,6,7-tetrahydro-1H-indazole-3-carboxamido)-1H-pyrazol-1-yl)piperidine-1-carboxylate (X₂₄a,)

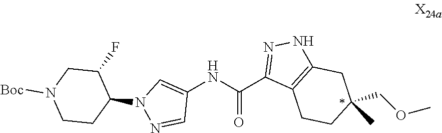 [SMILES: COC[C@]1(C)CCc2c(C(=O)Nc3cnn([C@H]4CCN(C(=O)OC(C)(C)C)C[C@@H]4F)c3)n[nH]c2C1]

Using general procedure H₁, (R)-6-(methoxymethyl)-6-methyl-4,5,6,7-tetrahydro-1H-indazole-3-carboxylic acid (30 mg, 0.14 mmol) was treated with tert-butyl (3S,4S)-4-(4-amino-1H-pyrazol-1-yl)-3-fluoropiperidine-1-carboxylate (45 mg, 0.16 mmol) to afford the title compound X²⁴a, *arbitrarily assigned (60 mg, 0.12 mmol, 85%). LCMS: C₂₄H₃₅FN₆O₄ requires: 490, found: m/z=491 [M+H]⁺.

### Example 108. tert-butyl (3R,4R)-3-fluoro-4-(4-((S)-6-(methoxymethyl)-6-methyl-4,5,6,7-tetrahydro-1H-indazole-3-carboxamido)-1H-pyrazol-1-yl)piperidine-1-carboxylate (X₂₅a)

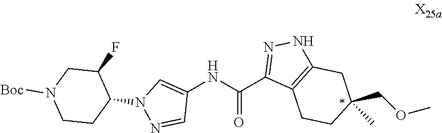 [SMILES: COC[C@@]1(C)CCc2c(C(=O)Nc3cnn([C@@H]4CCN(C(=O)OC(C)(C)C)C[C@H]4F)c3)n[nH]c2C1]

Using general procedure H₁, (S)-6-(methoxymethyl)-6-methyl-4,5,6,7-tetrahydro-1H-indazole-3-carboxylic acid (30 mg, 0.14 mmol) was treated with tert-butyl (3R,4R)-4-(4-amino-1H-pyrazol-1-yl)-3-fluoropiperidine-1-carboxylate (44 mg, 0.16 mmol) to afford the title compound X²⁵a, *arbitrarily assigned (52 mg, 0.11 mmol, 74%). LCMS: C₂₄H₃₅FN₆O₄ requires: 490, found: m/z=491 [M+H]⁺.

### Example 109 tert-butyl (3R,4R)-3-fluoro-4-(4-((R)-6-(methoxymethyl)-6-methyl-4,5,6,7-tetrahydro-1H-indazole-3-carboxamido)-1H-pyrazol-1-yl)piperidine-1-carboxylate (X₂₆a)

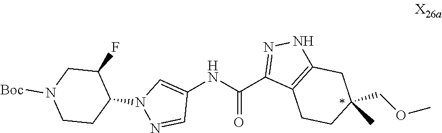 [SMILES: COC[C@]1(C)CCc2c(C(=O)Nc3cnn([C@@H]4CCN(C(=O)OC(C)(C)C)C[C@H]4F)c3)n[nH]c2C1]

Using general procedure H1, (R)-6-(methoxymethyl)-6-methyl-4,5,6,7-tetrahydro-1H-indazole-3-carboxylic acid (30 mg, 0.14 mmol) was treated with tert-butyl (3R,4R)-4-(4-amino-1H-pyrazol-1-yl)-3-fluoropiperidine-1-carboxylate (45 mg, 0.16 mmol) to afford the title compound X²⁶a (64 mg, 0.13 mmol, 91%). LCMS: C₂₄H₃₅FN₆O₄ requires: 490, found: m/z=491 [M+H]⁺.

### Example 110. tert-butyl 4-(4-((4aS,5aR)-5,5-difluoro-5a-methyl-1,4,4a,5,5a,6-hexahydrocyclopropa[f]indazole-3-carboxamido)phenyl)piperidine-1-carboxylate (X₂₇a)

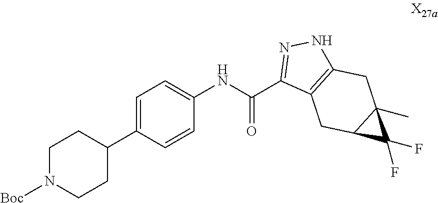 [SMILES: CC(C)(C)OC(=O)N1CCC(c2ccc(NC(=O)c3n[nH]c4c3C[C@@H]3C(F)(F)[C@]3(C)C4)cc2)CC1]

Using general procedure H₁, (4aS,5aR)-5,5-difluoro-5a-methyl-1,4,4a,5,5a,6-hexahydrocyclopropa[f]indazole-3-carboxylic acid (100 mg, 0.44 mmol) was treated with tert-butyl 4-(4-aminophenyl)piperidine-1-carboxylate (182 mg, 0.66 mmol) to afford the title compound X²⁷a (107 mg, 0.22 mmol, 50%). LCMS: C₂₆H₃₂F₂N₄O₃ requires: 486, found: m/z=487 [M+H]⁺.

### Example 111. tert-butyl 4-(5-((4aS,5aR)-5,5-difluoro-5a-methyl-1,4,4a,5,5a,6-hexahydrocyclopropa[f]indazole-3-carboxamido)pyridin-2-yl)piperidine-1-carboxylate (X₂₈ₐ)

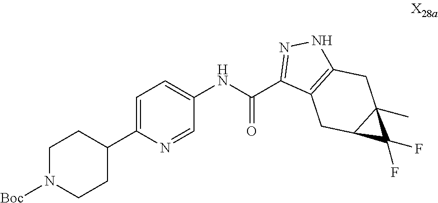 [SMILES: CC(C)(C)OC(=O)N1CCC(c2ccc(NC(=O)c3n[nH]c4c3C[C@@H]3C(F)(F)[C@]3(C)C4)cn2)CC1]

Using general procedure H₁, (4aS,5aR)-5,5-difluoro-5a-methyl-1,4,4a,5,5a,6-hexahydrocyclopropa[f]indazole-3-carboxylic acid (100 mg, 0.44 mmol) was treated with tert-butyl 4-(5-aminopyridin-2-yl)piperidine-1-carboxylate (182 mg, 0.66 mmol) to afford the title compound X₂₈ₐ (174 mg, 0.36 mmol, 81%). LCMS: C₂₅H₃₁F₂N₅O₃ requires: 487, found: m/z=488 [M+H]⁺.

### General Procedure H2: Boc Deprotection

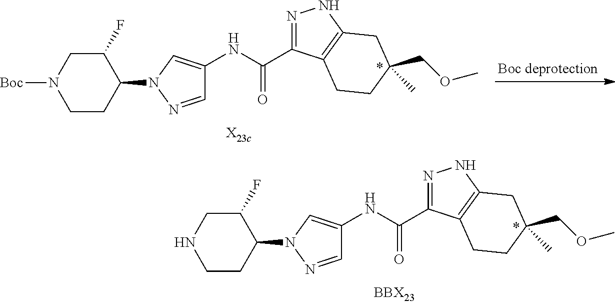 [SMILES: COC[C@@]1(C)CCc2c(C(=O)Nc3cnn([C@H]4CCN(C(=O)OC(C)(C)C)C[C@@H]4F)c3)n[nH]c2C1 ; COC[C@@]1(C)CCc2c(C(=O)Nc3cnn([C@H]4CCNC[C@@H]4F)c3)n[nH]c2C1]

### Scheme H2: Synthesis of BBX₂₈ via Boc Deprotection

A mixture of t-Bu carbamate X²³c (56 mg, 0.11 mmol) and TFA (175 μL) was stirred at rt for 1.5 hr. The resulting mixture was concentrated under a positive flow of N₂. Flash chromatography (C18, 5 to 75% MeCN/H₂O gradient elution) provided the amine product BBX₂₃ (49 mg, quant.). LCMS: C₁₉H₂₇FN₆O₂ requires: 390, found: m/z=391 [M+H]⁺. An exemplary Boc deprotection is provided in the Scheme H2 below where tert-butyl (3S,4S)-3-fluoro-4-(4-((S)-6-(methoxymethyl)-6-methyl-4,5,6,7-tetrahydro-1H-indazole-3-carboxamido)-1H-pyrazol-1-yl)piperidine-1-carboxylate (X_{23c}) was reacted as described above to provide (S)—N-(1-((3S,4S)-3-fluoropiperidin-4-yl)-1H-pyrazol-4-yl)-6-(methoxymethyl)-6-methyl-4,5,6,7-tetrahydro-1H-indazole-3-carboxamide (BBX₂₃) (*Arbitrarily Assigned)

### Example 112. (R)—N-(1-((3S,4S)-3-fluoropiperidin-4-yl)-1H-pyrazol-4-yl)-6-(methoxymethyl)-6-methyl-4,5,6,7-tetrahydro-1H-indazole-3-carboxamide (BBX₂₄) (*Arbitrarily Assigned)

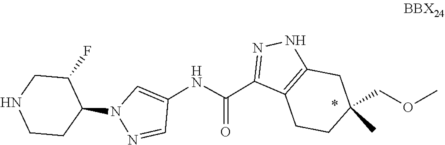 [SMILES: COC[C@]1(C)CCc2c(C(=O)Nc3cnn([C@H]4CCNC[C@@H]4F)c3)n[nH]c2C1]

Using general procedure H₂, tert-butyl (3S,4S)-3-fluoro-4-(4-((R)-6-(methoxymethyl)-6-methyl-4,5,6,7-tetrahydro-1H-indazole-3-carboxamido)-1H-pyrazol-1-yl)piperidine-1-carboxylate X²⁴a (59 mg, 0.12 mmol) was treated to afford the title compound BBX₂₄ (34 mg, 0.10 mmol, 83%). LCMS: C₁₉H₂₇FN₆O₂ requires: 390, found: m/z=391 [M+H]⁺.

### Example 113. (S)—N-(1-((3R,4R)-3-fluoropiperidin-4-yl)-1H-pyrazol-4-yl)-6-(methoxymethyl)-6-methyl-4,5,6,7-tetrahydro-1H-indazole-3-carboxamide (BBX₂₅) (*Arbitrarily Assigned)

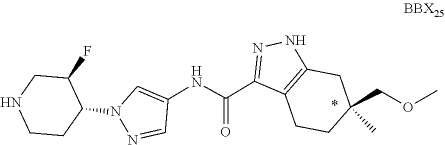 [SMILES: COC[C@@]1(C)CCc2c(C(=O)Nc3cnn([C@@H]4CCNC[C@H]4F)c3)n[nH]c2C1]

Using general procedure H₂, tert-butyl (3R,4R)-3-fluoro-4-(4-((S)-6-(methoxymethyl)-6-methyl-4,5,6,7-tetrahydro-1H-indazole-3-carboxamido)-1H-pyrazol-1-yl)piperidine-1-carboxylate X²⁵a (52 mg, 0.11 mmol) was treated to afford the title compound BBX₂₅ 38 mg, 0.098 mmol, 93%). LCMS: C₁₉H₂₇FN₆O₂ requires: 390, found: m/z=391 [M+H]⁺.

### Example 114. (R)—N-(1-((3R,4R)-3-fluoropiperidin-4-yl)-1H-pyrazol-4-yl)-6-(methoxymethyl)-6-methyl-4,5,6,7-tetrahydro-1H-indazole-3-carboxamide (BBX₂₆) (*Arbitrarily Assigned)

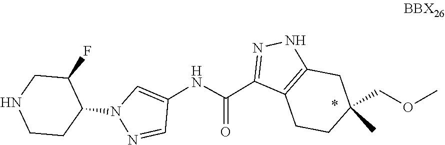 [SMILES: COC[C@]1(C)CCc2c(C(=O)Nc3cnn([C@@H]4CCNC[C@H]4F)c3)n[nH]c2C1]

Using general procedure H₂, tert-butyl (3R,4R)-3-fluoro-4-(4-((R)-6-(methoxymethyl)-6-methyl-4,5,6,7-tetrahydro-1H-indazole-3-carboxamido)-1H-pyrazol-1-yl)piperidine-1-carboxylate X²⁶a (64 mg, 0.13 mmol) was treated to afford the title compound BBX₂₆ (44 mg, 0.11 mmol, 87%). LCMS: C₁₉H₂₇FN₆O₂ requires: 390, found: m/z=391 [M+H]⁺.

### Example 115. (4aS,5aR)-5,5-difluoro-5a-methyl-N-(4-(piperidin-4-yl)phenyl)-1,4,4a,5,5a,6-hexahydrocyclopropa[f]indazole-3-carboxamide (BBX₂₇)

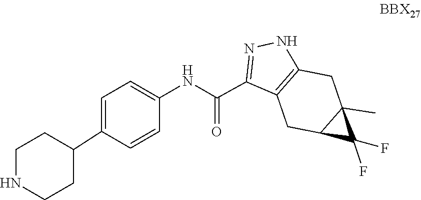 [SMILES: C[C@@]12Cc3[nH]nc(C(=O)Nc4ccc(C5CCNCC5)cc4)c3C[C@@H]1C2(F)F]

Using general procedure H₂, tert-butyl 4-(4-((4aS,5aR)-5,5-difluoro-5a-methyl-1,4,4a,5,5a,6-hexahydrocyclopropa[f]indazole-3-carboxamido)phenyl)piperidine-1-carboxylate X²⁷a (107 mg, 0.22 mmol) was treated to afford the title compound BBX₂₇. LCMS: C₂₁H₂₄F₂N₄O requires: 386, found: m/z=387 [M+H]⁺.

### Example 116. (4aS,5aR)-5,5-difluoro-5a-methyl-N-(6-(piperidin-4-yl)pyridin-3-yl)-1,4,4a,5,5a,6-hexahydrocyclopropa[f]indazole-3-carboxamide (BBX₂₈)

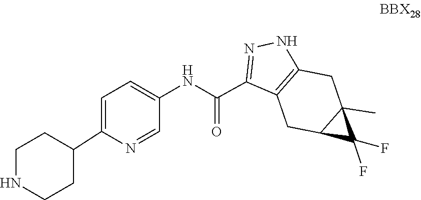 [SMILES: C[C@@]12Cc3[nH]nc(C(=O)Nc4ccc(C5CCNCC5)nc4)c3C[C@@H]1C2(F)F]

Using general procedure H₂, tert-butyl 4-(5-((4aS,5aR)-5,5-difluoro-5a-methyl-1,4,4a,5,5a,6-hexahydrocyclopropa[f]indazole-3-carboxamido)pyridin-2-yl)piperidine-1-carboxylate X₂₈ₐ (173 mg, 0.35 mmol) was treated to afford the title compound BBX₂₈. LCMS: C₂₀H₂₃F₂N₅O requires: 387, found: m/z=388 [M+H]⁺.

### Example 117. 2-[(4aS,5aR)-5,5-difluoro-5a-methyl-1H,4H,4aH,5H,5aH,6H-cyclopropa[f]indazol-3-yl]-6-[(3S)-3-methylpiperazine-1-carbonyl]-1H-indole (BBX₂₉)

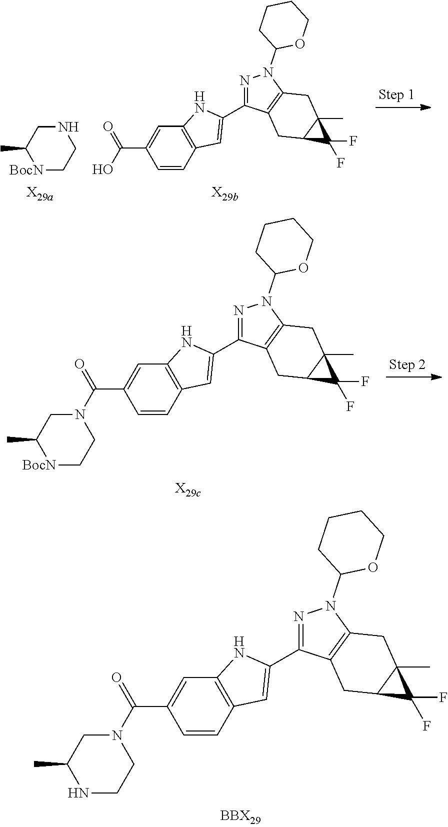

### Step 1: Synthesis of tert-butyl (2S)-4-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indole-6-carbonyl)-2-methylpiperazine-1-carboxylate (X_{29c})

To a 20 mL vial was added tert-butyl (2S)-2-methylpiperazine-1-carboxylate (X₂₉a) (75 mg, 0.37 mmol), 2-[(4aS,5aR)-5,5-difluoro-5a-methyl-1-(oxan-2-yl)-4H,4aH,6H-cyclopropa[f]indazol-3-yl]-1H-indole-6-carboxylic acid (X₂₉b) (160 mg, 1 eq), DIEA (0.56 mL, 8.5 eq), and DMF (1.5 mL). The mixture was stirred until full dissolution of solids. To the reaction mixture was added a solution of [(dimethylamino)({[1,2,3]triazolo[4,5-b]pyridin-3-yloxy})methylidene]dimethylazanium; hexafluoro-lambda5-phosphanuide (145 mg, 0.38 mmol) in DMF (0.4 mL). After 1 h, the reaction mixture was diluted with water. The product was extracted with EtOAC (3×), dried over MgSO₄, then concentrated. The resulting residue was purified by column chromatography (silica, 0-70% ethyl acetate in hexane) to afford tert-butyl (2S)-4-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indole-6-carbonyl)-2-methylpiperazine-1-carboxylate (X_{29c}).

### Step 2: Synthesis of 2-[(4aS,5aR)-5,5-difluoro-5a-methyl-1H,4H,4aH,5H,5aH,6H-cyclopropa[f]indazol-3-yl]-6-[(3S)-3-methylpiperazine-1-carbonyl]-1H-indole (BBX₂₉)

tert-butyl (2S)-4-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indole-6-carbonyl)-2-methylpiperazine-1-carboxylate (X_{29c}) and para-toluene sulfonate hydrate (0.10 mL, 124.79 mg, 0.66 mmol) were stirred in at 75C for 5 h. The mixture was cooled. Water and a little sat. aq. NaHCO₃ was added. The mixture was extracted three times with 10% MeOH/DCM. The combined organic layers were dried over Na₂SO₄ and concentrated to provide crude material. Chromatography (silica, 0-10% methanol in DCM) provided 2-[(4aS,5aR)-5,5-difluoro-5a-methyl-1H,4H,4aH,5H,5aH,6H-cyclopropa[f]indazol-3-yl]-6-[(3S)-3-methylpiperazine-1-carbonyl]-1H-indole (BBX₂₉) (50 mg, 36%). LCMS: C₂₃H₂₅F₂N₅O requires: 425.2, found: m/z=426.3 [M+H]⁺. ¹H NMR (500 MHz, Methanol-d₄) δ 7.66 (d, J=8.1 Hz, 1H), 7.56 (s, 1H), 7.13 (d, J=8.1 Hz, 1H), 6.74 (s, 1H), 3.26-3.16 (m, 3H), 3.16-3.07 (m, 3H), 3.04-2.93 (m, 2H), 2.85 (dd, J=16.9, 3.2 Hz, 1H), 1.85-1.68 (m, 1H), 1.44 (s, 3H), 1.27-1.13 (m, 3H).

### Example 118. 2-[(4aS,5aR)-5,5-difluoro-5a-methyl-1H,4H,4aH,5H,5aH,6H-cyclopropa[f]indazol-3-yl]-6-{3,8-diazabicyclo[3.2.1]octane-3-carbonyl}-1H-indole (BBX₃₀)

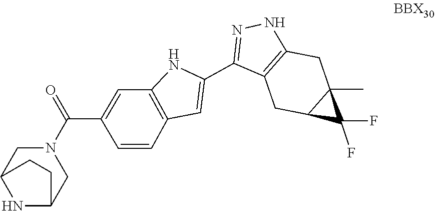 [SMILES: C[C@@]12Cc3[nH]nc(-c4cc5ccc(C(=O)N6CC7CCC(C6)N7)cc5[nH]4)c3C[C@@H]1C2(F)F]

Following the procedure for BBX₂₉ and starting from tert-butyl 3,8-diazabicyclo[3.2.1]octane-8-carboxylate provided 2-[(4aS,5aR)-5,5-difluoro-5a-methyl-1H,4H,4aH,5H,5aH,6H-cyclopropa[f]indazol-3-yl]-6-{3,8-diazabicyclo[3.2.1]octane-3-carbonyl}-1H-indole (BBX₃₀). LCMS: C₂₄H₂₅F₂N₅O requires: 437.2, found: m/z=438.3 [M+H]⁺. ¹H NMR (500 MHz, Methanol-d₄) δ 7.67 (d, J=8.1 Hz, 1H), 7.57 (s, 1H), 7.12 (d, J=8.1 Hz, 1H), 6.74 (s, 1H), 4.61 (s, 1H), 3.96 (s, 2H), 3.26-3.01 (m, 3H), 2.85 (dd, J=16.9, 3.2 Hz, 1H), 2.14-1.86 (m, 4H), 1.85-1.66 (m, 1H), 1.45 (s, 3H).

### Example 119. (4aS,5aR)-5,5-difluoro-5a-methyl-N-{1-[(1rs,4rs)-4-formylcyclohexyl]pyrazol-4-yl}-1H,4H,4aH,6H-cyclopropa[f]indazole-3-carboxamide (BBX₃₁)

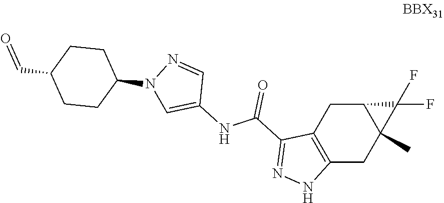 [SMILES: C[C@@]12Cc3[nH]nc(C(=O)Nc4cnn([C@H]5CC[C@H](C=O)CC5)c4)c3C[C@@H]1C2(F)F]

Following the procedure for BBX₂₉) and starting from (4aS,5aR)-5,5-difluoro-5a-methyl-1,4,4a,5,5a,6-hexahydrocyclopropa[f]indazole-3-carboxylic acid provided (4aS,5aR)-5,5-difluoro-5a-methyl-N-{1-[(1rs,4rs)-4-formylcyclohexyl]pyrazol-4-yl}-1H,4H,4aH,6H-cyclopropa[f]indazole-3-carboxamide (BBX₃₁). LCMS: C₂₀H₂₃F₂N₅O₂ requires: 403.4, found: m/z=404.4 [M+H]⁺.

### Example 120. (2-((4aS,5aR)-5,5-difluoro-5a-methyl-1,4,4a,5,5a,6-hexahydrocyclopropa[f]indazol-3-yl)-1H-indol-6-yl)((R)-3-methylpiperazin-1-yl)methanone (BBX₃₂)

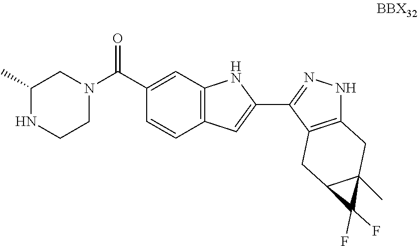

### Step 1: Synthesis of tert-butyl (2R)-4-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indole-6-carbonyl)-2-methylpiperazine-1-carboxylate (X₃₂b)

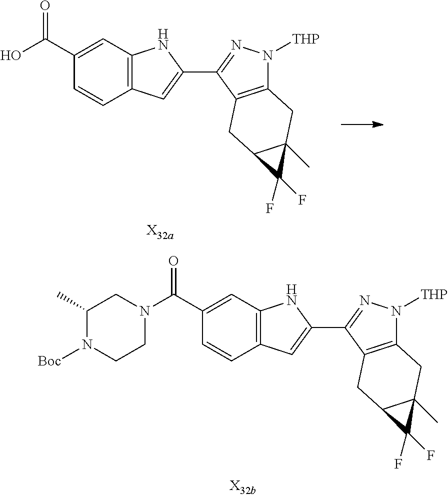

A mixture of 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indole-6-carboxylic acid (X₃₂ₐ) (300 mg, 0.70 mmol), tert-butyl (R)-2-methylpiperazine-1-carboxylate (155 mg, 0.77 mmol), EDCI (148 mg, 0.77 mmol) and HOBt (118 mg, 0.77 mmol) in DMF (3.5 mL) was allowed to stir at rt for 16 hr. NaBH(OAc)₃ (212 mg, 1.0 mmol) was added, and the reaction mixture was allowed to stir at rt for 16 hr. The reaction was quenched with H₂O. The reaction mixture was extracted with EtOAc, washed with saturated aqueous NaCl, dried over Na₂SO₄, and concentrated under reduced pressure. Flash chromatography (SiO₂, 0 to 100% EtOAc/hexanes gradient elution) provided tert-butyl (2R)-4-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indole-6-carbonyl)-2-methylpiperazine-1-carboxylate (BBX₃₂) (407 mg, 0.67 mmol, 95%). LCMS: C₃₃H₄₁F₂N₅O₄ requires: 609, found: m/z=610 [M+H]⁺.

### Step 2: Synthesis of (2-((4aS,5aR)-5,5-difluoro-5a-methyl-1,4,4a,5,5a,6-hexahydrocyclopropa[f]indazol-3-yl)-1H-indol-6-yl)((R)-3-methylpiperazin-1-yl)methanone (BBX₃₂)

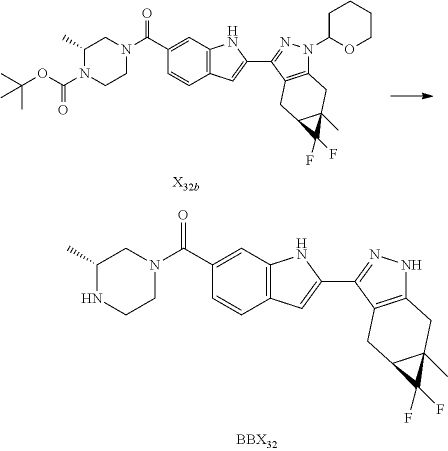

tert-butyl (2R)-4-{2-[(4aS,5aR)-5,5-difluoro-5a-methyl-1-(oxan-2-yl)-4H,4aH,6H-cyclopropa[f]indazol-3-yl]-1H-indole-6-carbonyl}-2-methylpiperazine-1-carboxylate (X₃₂b) (401 mg, 0.66 mmol) was stirred in hydrogen chloride (5.00 mL, 0.73 g, 20.00 mmol) 4.0M solution in dioxane for 90 minutes. The mixture was concentrated to provide (2-((4aS,5aBBX53R)-5,5-difluoro-5a-methyl-1,4,4a,5,5a,6-hexahydrocyclopropa[f]indazol-3-yl)-1H-indol-6-yl)((R)-3-methylpiperazin-1-yl)methanone hydrochloride (0.304 g, 100%). LCSM C₂₃H₂₅F₂N₅O requires: 425, found: m/z=426 [M+H]⁺.

### Example 121. (4aS,5aR)-5,5-difluoro-5a-methyl-N-[1-(piperidin-4-yl)-1H-pyrazol-4-yl]-1H,4H, 4aH,5H,5aH,6H-cyclopropa[f]indazole-3-carboxamide hydrochloride (BBX₃₃)

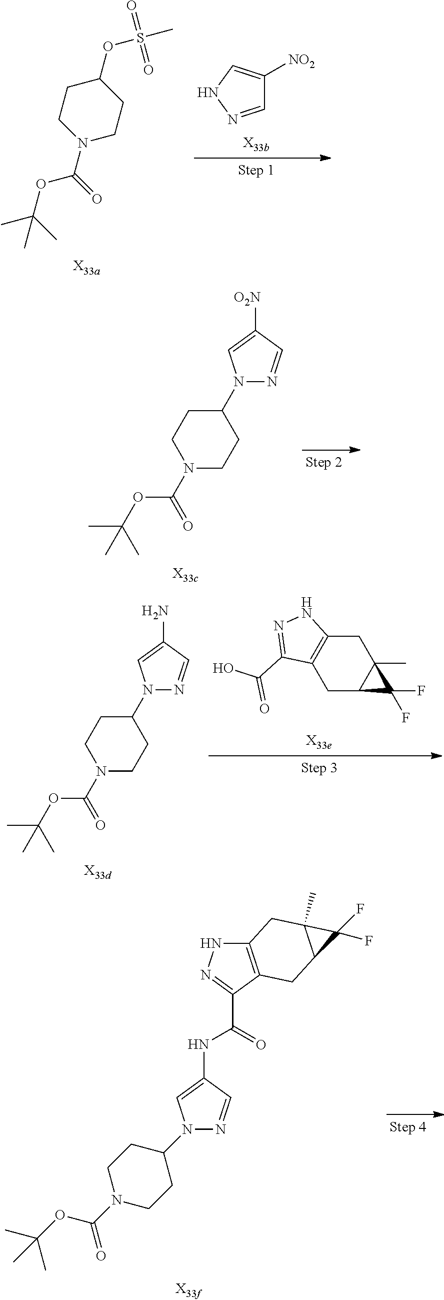

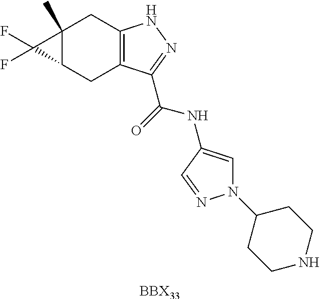

### Step 1: Synthesis of tert-butyl 4-(4-nitro-1H-pyrazol-1-yl)piperidine-1-carboxylate (X_{33c})

Under an argon atmosphere, to a solution of 4-nitro-1H-pyrazole (X_{33b}) (10 g, 88.45 mmol, 1.2 eq) in N-methyl-2-pyrrolidone (184 mL, 0.4 M) was added cesium carbonate (48.03 g, 147.41 mmol, 2.0 eq) at RT. Then, to the reaction mixture was added tert-butyl 4-[(methanesulfonyloxy)methyl]piperidine-1-carboxylate (X₃₃a) (20.59 g, 73.70 mmol, 1.0 eq), and the mixture was further stirred for 16 hr at 120° C. After completion of the reaction, it was diluted with ethyl acetate (250 mL) and water (250 mL), and the organic layer was separated. Then, the organic layer was washed successively with water and saturated brine, dried over sodium sulfate. Sodium sulfate was removed by filtration, and the filtrate was concentrated under reduced pressure to yield tert-butyl 4-(4-nitro-1H-pyrazol-1-yl)piperidine-1-carboxylate (X_{33c}) (74 g) of the crude. The residue was purified by silica gel chromatography (hexane/EtOAc 0 to 100%) to give tert-butyl 4-(4-nitro-1H-pyrazol-1-yl)piperidine-1-carboxylate (X_{33c}) (16.3 g, 55 mmol, 74% yield) as a white solid. Note: By using Et₂O for extraction, we can eliminate unreacted 4-nitro-pyrazole and column purification can be avoided. However, the elimination product derived from mesyl derivative will be present in the product which can be removed by trituration with Et₂O after reduction or after amide coupling. ¹H NMR (300 MHz, Chloroform-d) δ 8.18 (s, 1H), 8.13-8.07 (m, 1H), 4.31 (m, 3H), 3.02-2.80 (m, 2H), 2.26-2.08 (m, 2H), 1.92 (dd, J=12.2, 4.4 Hz, 2H), 1.50 (s, 9H). LCMS (254 nm): method: 205_254_m_z_100-1000 ALT ig45_6 min_nonpolar.1 cm, RT: 2.82 min, purity: 98.90%, ESI(−) [M−H]⁻ 295.1

### Step 2: Synthesis of tert-butyl 4-(4-amino-1H-pyrazol-1-yl)piperidine-1-carboxylate (X_{33d})

Palladium hydroxide on carbon (1.63 g, 10% wt) was added to a solution of tert-butyl 4-(4-nitro-1H-pyrazol-1-yl)piperidine-1-carboxylate (X_{33c}) (16.3 g, 54.46 mmol, 1 eq) in methanol (16.3 mL, 0.04 M). The mixture reaction was purged 3 times with vacuum/hydrogen cycles and left stirring under a hydrogen atmosphere (1 atm) for 10 hours at room temperature. The reaction was filtered through a celite pad, washed with MeOH (3×100 mL), evaporated to dryness to give dark oil which was dissolved with Et₂O (100 mL), and concentrated back (×2) to remove MeOH. The resulting crude, tert-butyl 4-(4-amino-1H-pyrazol-1-yl)piperidine-1-carboxylate (X_{33d}) (15 g) was used for the next step without further purification. ¹H NMR (300 MHz, Chloroform-d) δ 7.18 (d, J=1.0 Hz, 1H), 7.06 (d, J=0.9 Hz, 1H), 4.32-4.07 (m, 3H), 2.98-2.80 (m, 3H), 2.09 (d, J=12.8 Hz, 2H), 1.86 (m, 3H), 1.49 (s, 9H). LCMS (254 nm): method: 254_280_m_z_100-1000 ALT ig45_6 min_standard.1 cm, ESI(+) [M+H]⁺=267.4 [M+H]⁺.

### Step 3: Synthesis of tert-butyl 4-{4-[(4aS,5aR)-5,5-difluoro-5a-methyl-1H,4H,4aH,5H,5aH,6H-cyclopropa[f]indazole-3-amido]-1H-pyrazol-1-yl}piperidine-1-carboxylate (X_{33f})

To a mixture of the (4aS,5aR)-5,5-difluoro-5a-methyl-1H,4H,4aH,5H,5aH,6H-cyclopropa[f]indazole-3-carboxylic acid (X₃₃ₑ) (1.8 g, 7.57 mmol, 1.0 eq), triethylamine (3.2 ml, 22.72 mmol, 3.0 eq) and the tert-butyl 4-(4-amino-1H-pyrazol-1-yl)piperidine-1-carboxylate (X₃₃a) (2.5 g, 9.09 mmol, 1.2 eq) in ethyl acetate (38 ml, 0.2 M) was slowly added T3P (50% in EtOAc, 3.61 ml, 11.36 mmol, 1.5 eq) at 0° C. The reaction mixture was slowly allowed to warm to room temperature and stirred for 10 h. LCMS and TLC analyses showed the product formation. The reaction mixture was chilled on an ice-water bath, water (100 mL) was added dropwise. The mixture was left stirring for 25 min at room temperature, diluted with EtOAc (100 mL), and layers were separated (aq pH-6). The organic layer was washed with sat aq NaHCO₃ (2×35 mL), brine (35 mL), dried over Na₂SO₄, and evaporated to give 4.1 g of crude as a pale brown foam. The residue was purified to silica gel flash chromatography (MeOH/DCM 0 to 2.5%) to give two lots with a slight difference in purity to the total of 2.3 g (4.82 mmol, 63% yield) of tert-butyl 4-{4-[(4aS,5aR)-5,5-difluoro-5a-methyl-1H,4H,4aH,5H,5aH,6H-cyclopropa[f]indazole-3-amido]-1H-pyrazol-1-yl}piperidine-1-carboxylate (X_{33f}) as a pale brown foam. Note: Flash chromatography can be avoided by careful trituration of the crude with Et₂O. The two lots were subjected to Boc deprotection separately. ¹H NMR (300 MHz, DMSO-d6) δ 12.97 (s, 1H), 10.14 (s, 1H), 8.02 (s, 1H), 7.64 (s, 1H), 4.40-4.22 (m, 1H), 4.03 (d, J=13.2 Hz, 2H), 3.07 (s, 2H), 3.03-2.97 (m, 1H), 2.97-2.76 (m, 3H), 2.03-1.88 (m, 3H), 1.73 (qd, J=12.2, 4.2 Hz, 3H), 1.42 (s, 10H), 1.35 (d, J=2.5 Hz, 3H). LCMS (254 nm): method: LCMS2-036-20-80-95-6-1-25-UV-BCM, Rt=3.10 min, purity: lot #1: 92%; lot #2: 96%. ESI (+) [M+Na]=499.42.

### Step 4: Synthesis of (4aS,5aR)-5,5-difluoro-5a-methyl-N-[1-(piperidin-4-yl)-1H-pyrazol-4-yl]-1H,4H, 4aH,5H,5aH,6H-cyclopropa[f]indazole-3-carboxamide hydrochloride (BBX₃₃)

To a solution of the tert-butyl 4-{4-[(4aS,5aR)-5,5-difluoro-5a-methyl-1H,4H,4aH,5H,5aH,6H-cyclopropa[f]indazole-3-amido]-1H-pyrazol-1-yl}piperidine-1-carboxylate (X_{33f}) (1.2 g, 2.42 mmol, 1.0 eq) in a mixture of anhydrous dichloromethane (30 ml, 0.08 M) and methanol (3 mL) was slowly added 4M HCl in 1,4-dioxane (4.23 mL, 16.92 mmol, 7.0 eq). The reaction mixture was stirred at room temperature for 4 h. The solid was filtered off, washed 3 times with Et₂O on the filter, and dried in vacuo overnight to yield (4aS,5aR)-5,5-difluoro-5a-methyl-N-[1-(piperidin-4-yl)-1H-pyrazol-4-yl]-1H,4H, 4aH,5H,5aH,6H-cyclopropa[f]indazole-3-carboxamide hydrochloride (BBX₃₃) (890 mg, 2.15 mmol, 89%) as a white solid. Note: Similarly, another lot of product (1.1 g) was subjected to deprotection to yield (4aS, 5aR)-5,5-difluoro-5a-methyl-N-[1-(piperidin-4-yl)-1H-pyrazol-4-yl]-1H,4H, 4aH,5H,5aH,6H-cyclopropa[f]indazole-3-carboxamide hydrochloride (BBX₃₃) (822 mg). ¹H NMR (300 MHz, DMSO-d6) δ 10.24 (s, 1H), 9.25 (s, 1H), 9.00 (d, J=11.2 Hz, 1H), 8.03 (s, 1H), 7.68 (s, 1H), 4.46 (p, J=7.6 Hz, 1H), 3.37 (d, J=12.5 Hz, 2H), 3.13-2.98 (m, 5H), 2.82 (dd, J=16.8, 3.4 Hz, 1H), 2.14 (m, 4H), 1.85-1.72 (m, 1H), 1.35 (d, J=2.5 Hz, 3H). LCMS (254 nm): method: LCMS2-036-5-80-80-7-1-25-UV-Rot, Rt=3.06 min, purity: 96%. ESI (+) [M+Na]=377.24.

### Example 122. (4aS,5aR)-5,5-difluoro-5a-methyl-N-{1-[(piperidin-4-yl)methyl]-1H-pyrazol-4-yl}-1H,4H,4aH,5H,5aH,6H-cyclopropa[f]indazole-3-carboxamide (BBX₃₄)

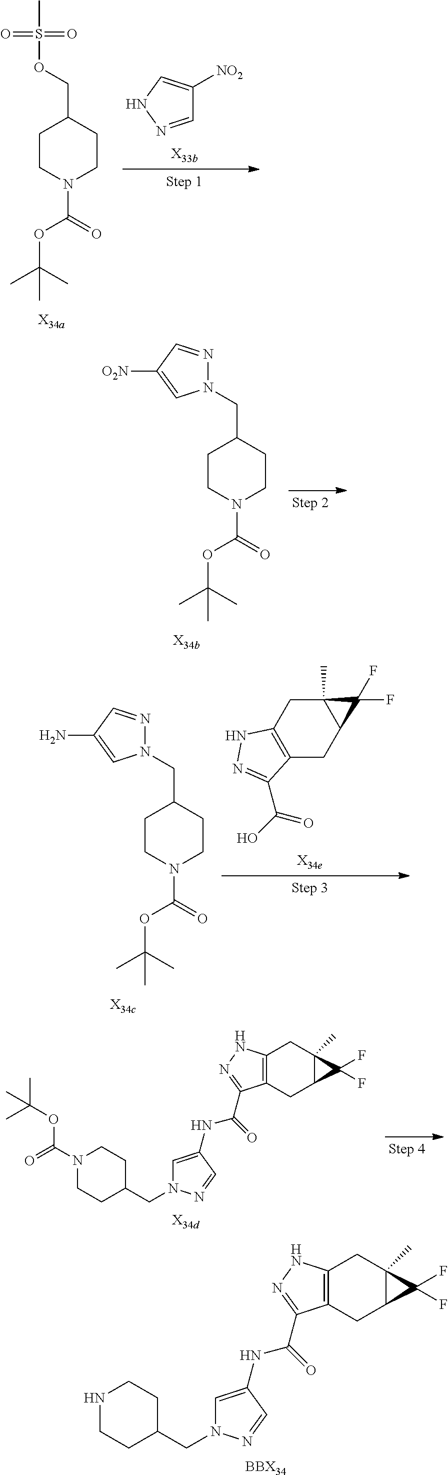

### Step 1: Synthesis of tert-butyl 4-[(4-nitro-1H-pyrazol-1-yl)methyl]piperidine-1-carboxylate (X_{34b})

Under an argon atmosphere, to a solution of 4-nitro-1H-pyrazole (X_{33b}) (0.925 g, 8.18 mmol, 1.2 eq) in dimethylformamide anhydrous (17.04 ml, 0.4 M) was added cesium carbonate (4.442 g, 13.633 mmol, 2.0 eq) at room temperature. Then, to the reaction mixture was added tert-butyl 4-[(methanesulfonyloxy)methyl]piperidine-1-carboxylate (X₃₄a) (2.0 g, 6.817 mmol, 1.0 eq), and the mixture was further stirred for 16 hr at 120° C. To the reaction mixture were added ethyl acetate and water, and the organic layer was separated. Then, the organic layer was washed successively with water and saturated brine, and dried over sodium sulfate. Sodium sulfate was removed by filtration, and the filtrate was concentrated under reduced pressure. The residue was purified by silica gel chromatography to give tert-butyl 4-[(4-nitro-1H-pyrazol-1-yl)methyl]piperidine-1-carboxylate (X_{34b}) (1.58 g, 75%) as a white solid. ESI(+) [M+H-Boc]⁺=211.2. ¹H NMR (300 MHz, Chloroform-d) δ 8.11 (d, J=2.0 Hz, 2H), 4.17 (d, J=13.4 Hz, 2H), 4.04 (d, J=7.2 Hz, 2H), 2.78-2.63 (m, 2H), 2.13 (ttt, J=10.5, 6.8, 3.4 Hz, 1H), 1.59 (d, J=11.7 Hz, 3H), 1.47 (s, 9H), 1.21 (qd, J=12.4, 4.0 Hz, 3H).

### Step 2: Synthesis of tert-butyl 4-[(4-amino-1H-pyrazol-1-yl)methyl]piperidine-1-carboxylate (X_{34c})

To a solution of tert-butyl 4-[(4-nitro-1H-pyrazol-1-yl)methyl]piperidine-1-carboxylate (X_{34b}) (1.58 g, 5.09 mmol, 1.0 eq) in methanol (196 ml, 0.026 M) was added 10% wt Pd(OH)₂/C (0.363 g, 0.255 mmol, 0.05 eq). The mixture reaction was purged 3 times with vacuum/hydrogen cycles and left stirring under hydrogen atmosphere for 60 hours at room temperature. The mixture was filtered through celite pad, washed with MeOH (3×25 mL), evaporated to give 1.5 g of crude as a dark grey solid (NMR shows −20% of semireduced intermediate). The residue was purified by silica gel chromatography (80 g, DCM/MeOH 0-10%) to give tert-butyl 4-[(4-amino-1H-pyrazol-1-yl)methyl]piperidine-1-carboxylate (X_{34c}) (1.15 g, 81%) as a yellow solid. ESI(+) [M+H]⁺=281.5. ¹H NMR (300 MHz, Chloroform-d) δ 7.20 (d, J=0.9 Hz, 1H), 7.02 (d, J=0.9 Hz, 1H), 4.20-4.07 (m, 2H), 3.87 (d, J=7.2 Hz, 2H), 3.19 (d, J=46.1 Hz, 2H), 2.67 (t, J=12.7 Hz, 2H), 2.00 (m, 1H), 1.56 (d, J=12.9 Hz, 2H), 1.46 (s, 9H), 1.16 (qd, J=12.3, 4.4 Hz, 2H).

### Step 3: Synthesis of tert-butyl 4-({4-[(4aS,5aR)-5,5-difluoro-5a-methyl-1H,4H,4aH,5H,5aH,6H-cyclopropa[f]indazole-3-amido]-1H-pyrazol-1-yl}methyl)piperidine-1-carboxylate (X₃₄a)

To a mixture of the (4aS,5aR)-5,5-difluoro-5a-methyl-1H,4H,4aH,5H,5aH,6H-cyclopropa[f]indazole-3-carboxylic acid (X₃₃ₑ) (0.894 g, 3.918 mmol, 1.0 eq), triethylamine (1.638 ml, 11.752 mmol, 3.0 eq) and the tert-butyl 4-[(4-amino-1H-pyrazol-1-yl)methyl]piperidine-1-carboxylate (X_{34c}) (1.153 g, 4.112 mmol, 1.05 eq) in ethyl acetate (19.59 ml, 0.2 M) was slowly added T3P (50% in EtOAc, 3.498 ml, 5.876 mmol, 1.5 eq) at 0° C. The reaction mixture was slowly allowed to warm to RT and stirred for 10 h. The reaction mixture was chilled on ice-water bath, water (10 mL) was added dropwise, let stirring for 25 min at RT, diluted with EtOAc (50 mL) and layers were separated (aq pH-6). The organic layer was washed with sat NaHCO₃ (2×25 mL), brine (25 mL), dried over Na₂SO₄, filtered, and evaporated to give 1.56 g of semisolid crude. The crude material was subjected to flash chromatography (Hex/EtOAc 50->100%); fraction containing the product was evaporated to dryness to provide tert-butyl 4-({4-[(4aS,5aR)-5,5-difluoro-5a-methyl-1H,4H,4aH,5H,5aH,6H-cyclopropa[f]indazole-3-amido]-1H-pyrazol-1-yl}methyl)piperidine-1-carboxylate (X₃₄a) (1.0 g, 50%) as a colorless solid. ESI (+) [M+H]⁺=491.2. ¹H NMR (300 MHz, DMSO-d6) δ 8.72 (s, 1H), 8.04 (s, 1H), 7.56 (d, J=0.8 Hz, 1H), 5.96 (s, 2H), 4.14 (m, 2H), 3.99 (d, J=7.2 Hz, 2H), 3.35 (d, J=17.7 Hz, 1H), 3.29-3.07 (m, 2H), 2.79 (dd, J=17.0, 3.3 Hz, 1H), 2.68 (t, J=12.7 Hz, 2H), 2.17-2.00 (m, 1H), 1.66 (d, J=12.6 Hz, 1H), 1.58 (d, J=14.3 Hz, 3H), 1.47 (s, 9H), 1.18 (m, 2H).

### Step 4: Synthesis of (4aS,5aR)-5,5-difluoro-5a-methyl-N-{1-[(piperidin-4-yl)methyl]-1H-pyrazol-4-yl}-1H,4H,4aH,5H,5aH,6H-cyclopropa[f]indazole-3-carboxamide (BBX₃₄)

To a solution of the tert-butyl 4-({4-[(4aS,5aR)-5,5-difluoro-5a-methyl-1H,4H,4aH,5H,5aH,6H-cyclopropa[f]indazole-3-amido]-1H-pyrazol-1-yl}methyl)piperidine-1-carboxylate (1.0 g, 2.039 mmol, 1.0 eq) in dichloromethane anhydrous (25.48 ml, 0.08 M) was slowly added 2M HCl in diethyl ether (7.135 ml, 14.27 mmol, 7.0 eq). The reaction mixture was stirred at RT for 2 h. The solid was filtered off, washed 3 times with Et₂O on the filter and dried in vacuo overnight to provide 688 mg (86% yield) of the title compound as a white solid. LCMS: C₁₉H₂₄F₂N₆O requires: 390.2, found: m/z=391.0 [M+H]⁺, method: LCMS2-036-10-60-95-6-1-25-UV, RT=2.067 min, 95.33% purity (254 nm); ¹H NMR (300 MHz, DMSO-d6) δ 10.20 (s, 1H), 8.83 (d, J=11.1 Hz, 1H), 8.48 (d, J=11.4 Hz, 1H), 8.05 (s, 1H), 7.63 (s, 1H), 4.01 (d, J=6.8 Hz, 2H), 3.23 (d, J=12.5 Hz, 2H), 3.10-3.05 (m, 2H), 3.01 (s, 1H), 2.88-2.73 (m, 3H), 2.08 (d, J=2.8 Hz, 1H), 1.83-1.72 (m, 1H), 1.68-1.57 (m, 2H), 1.35 (m, 5H).

### Example 123. Synthesis of (4aS,5aR)-5,5-difluoro-N-{1-[(3S,4S)-3-fluoropiperidin-4-yl]pyrazol-4-yl}-5a-methyl-1H,4H,4aH,6H-cyclopropa[f]indazole-3-carboxamide (BBX₃₅)

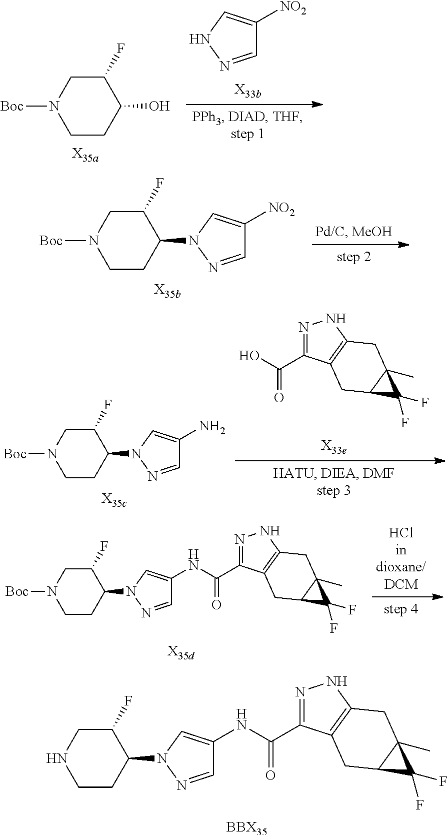

### Step 1: Synthesis of tert-butyl (3S,4S)-3-fluoro-4-(4-nitropyrazol-1-yl)piperidine-1-carboxylate (X_{35b})

To a stirred mixture of tert-butyl (3S,4R)-3-fluoro-4-hydroxypiperidine-1-carboxylate (X₃₅a) (1 g, 4.561 mmol, 1 equiv), 4-nitropyrazole (X_{33b}) (515.73 mg, 4.561 mmol, 1 equiv) and PPh₃ (1.44 g, 5.473 mmol, 1.2 equiv) in THF (10 mL) were added DIAD (1.20 g, 5.929 mmol, 1.3 equiv) at room temperature. The resulting mixture was stirred at room temperature overnight. The resulting mixture was concentrated under reduced pressure. The residue was purified by silica gel column chromatography, eluted with PE/EA (2/1) to afford tert-butyl (3S,4S)-3-fluoro-4-(4-nitropyrazol-1-yl)piperidine-1-carboxylate (X_{35b}) (1 g, 69.78%) as a yellow oil.

### Step 2: Synthesis of tert-butyl (3S,4S)-4-(4-aminopyrazol-1-yl)-3-fluoropiperidine-1-carboxylate (X_{35b})

To a stirred mixture of tert-butyl (3S,4S)-3-fluoro-4-(4-nitropyrazol-1-yl)piperidine-1-carboxylate (X_{35b}) (1 g, 3.182 mmol, 1 equiv) in MeOH (10 mL) was added Pd/C (500 mg). The resulting mixture was stirred at room temperature for 3.5 h under hydrogen atmosphere. The resulting mixture was filtered. The filtrate was concentrated under reduced pressure. This resulted in tert-butyl (3S,4S)-4-(4-aminopyrazol-1-yl)-3-fluoropiperidine-1-carboxylate (X_{35c}) (700 mg, crude) as a yellow oil. MS (ESI) calc'd for (C₁₃H₂₁FN₄O₂) [M+1]+, 285.1; found, 285.2.

### Step 3: Synthesis of tert-butyl (3S,4S)-4-{4-[(4aS,5aR)-5,5-difluoro-5a-methyl-1H,4H,4aH,6H-cyclopropa[f]indazole-3-amido]pyrazol-1-yl}-3-fluoropiperidine-1-carboxylate (X_{35d})

To a stirred mixture of tert-butyl (3S,4S)-4-(4-aminopyrazol-1-yl)-3-fluoropiperidine-1-carboxylate (X_{35c}) (373.80 mg, 1.315 mmol, 1.2 equiv), (4aS,5aR)-5,5-difluoro-5a-methyl-1H,4H,4aH,6H-cyclopropa[f]indazole-3-carboxylic acid (X₃₃ₑ) (250 mg, 1.096 mmol, 1.0 equiv) and HATU (624.8 mg, 1.644 mmol, 1.5 equiv) in DMF (3 mL) were added DIEA (424.8 mg, 3.288 mmol, 3 equiv) at room temperature. The mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The crude product was purified by reverse phase flash with the following conditions (H₂O/MeCN=1/1) to afford tert-butyl (3S,4S)-4-{4-[(4aS,5aR)-5,5-difluoro-5a-methyl-1H,4H,4aH,6H-cyclopropa[f]indazole-3-amido]pyrazol-1-yl}-3-fluoropiperidine-1-carboxylate (X_{35d}) (120 mg, 20.82%) as a white solid. MS (ESI) calc'd for (C₂₃H₂₉F₃N₆O₃) [M+1]⁺, 495.2; found, 495.2.

### Step 4: Synthesis of (4aS,5aR)-5,5-difluoro-N-{1-[(3S,4S)-3-fluoropiperidin-4-yl]pyrazol-4-yl}-5a-methyl-1H,4H,4aH,6H-cyclopropa[f]indazole-3-carboxamide (BBX₃₅)

To a stirred mixture of tert-butyl (3S,4S)-4-{4-[(4aS,5aR)-5,5-difluoro-5a-methyl-1H,4H,4aH,6H-cyclopropa[f]indazole-3-amido]pyrazol-1-yl}-3-fluoropiperidine-1-carboxylate (X_{35d}) (110 mg, 0.222 mmol, 1 equiv) in DCM (2 mL) was added HCl (gas) in 1,4-dioxane (2 mL, 4N) at room temperature. The mixture was stirred for 2 h at room temperature. This resulted in (4aS,5aR)-5,5-difluoro-N-{1-[(3S,4S)-3-fluoropiperidin-4-yl]pyrazol-4-yl}-5a-methyl-1H,4H,4aH,6H-cyclopropa[f]indazole-3-carboxamide (BBX₃₅) (91.2 mg, HCl salt) as a light yellow solid. MS (ESI) calc'd for (C₁₈H₂₁F₃N₆O) [M+1]⁺, 395.2; found, 395.2. ¹H NMR (400 MHz, Methanol-d₄) δ 8.16 (s, 1H), 7.77 (s, 1H), 5.22-5.12 (m, 1H), 4.77-4.75 (m, 1H), 3.86-3.82 (m, 1H), 3.68-3.60 (m, 2H), 3.49-3.39 (m, 1H), 3.32-3.31 (m, 1H), 3.19-3.17 (m, 1H), 3.17-3.09 (m, 1H), 2.85-2.81 (m, 1H), 2.45-2.43 (m, 2H), 1.73-1.71 (m, 1H), 1.44-1.43 (m, 3H).

### Example 124. (4aS,5aR)-5,5-difluoro-N-{1-[(3R,4R)-3-fluoropiperidin-4-yl]pyrazol-4-yl}-4,5a-dimethyl-1H,4H,4aH,6H-cyclopropa[f]indazole-3-carboxamide (BBX₃₆)

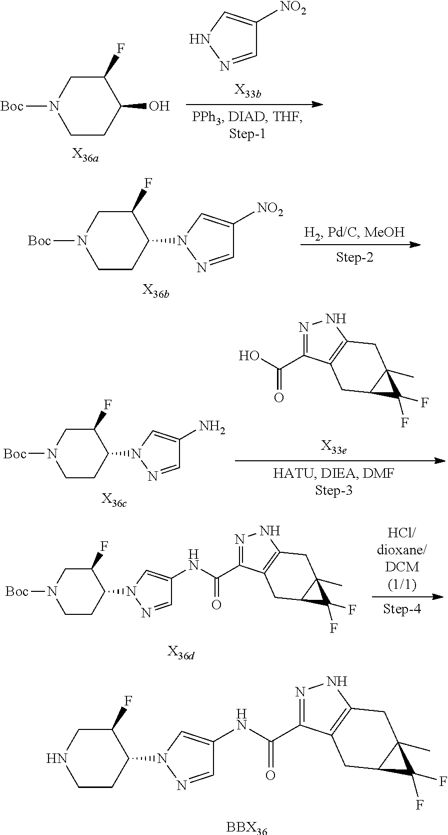

### Step 1: Synthesis of tert-butyl (3R,4R)-3-fluoro-4-(4-nitropyrazol-1-yl) piperidine-1-carboxylate (X_{36b})

To a stirred mixture of tert-butyl (3R,4S)-3-fluoro-4-hydroxypiperidine-1-carboxylate (X₃₆ₐ) (500 mg, 2.280 mmol, 1 equiv), 4-nitropyrazole (X_{33b}) (257.9 mg, 2.280 mmol, 1 equiv) and PPh₃ (717.8 mg, 2.736 mmol, 1.2 equiv) in THE (5 mL) added DIAD (599.5 mg, 2.964 mmol, 1.3 equiv) dropwise at 0° C. The resulting mixture was stirred at room temperature overnight. The resulting mixture was extracted with EtOAc. The combined organic layers were washed with water, dried over anhydrous Na₂SO₄. After filtration, the filtrate was concentrated under reduced pressure. The residue was purified by Prep-TLC (PE/EA 2/1) to afford tert-butyl (3R,4R)-3-fluoro-4-(4-nitropyrazol-1-yl) piperidine-1-carboxylate (X_{36b}) (420 mg, 58.60%) as a yellow oil.

### Step 2: Synthesis of tert-butyl (3R,4R)-4-(4-aminopyrazol-1-yl)-3-fluoropiperidine-1-carboxylate (X_{3rc})

To a stirred mixture of tert-butyl (3R,4R)-3-fluoro-4-(4-nitropyrazol-1-yl) piperidine-1-carboxylate (X_{36b}) (420 mg, 0.038 mmol, 1 equiv) in MeOH (5 mL) was added Pd/C (210 mg). The resulting mixture was stirred at room temperature for 3.5 h under hydrogen atmosphere. The resulting mixture was filtered. The filtrate was concentrated under reduced pressure. The residue was purified by silica gel column chromatography, eluted with CH₂Cl₂/MeOH (10/1) to afford tert-butyl (3R,4R)-4-(4-aminopyrazol-1-yl)-3-fluoropiperidine-1-carboxylate (X_{36c}) (310 mg, 81.59%) as a white solid. MS (ESI) calc'd for (C₁₃H₂₁FN₄O₂) [M+1]⁺, 285.2; found, 285.3.

### Step 3: Synthesis of tert-butyl (3R,4R)-4-{4-[(4aS,5aR)-5,5-difluoro-5a-methyl-1H,4H,4aH,6H-cyclopropa[f]indazole-3-amido]pyrazol-1-yl}-3-fluoropiperidine-1-carboxylate (X₃₆d)

To a stirred mixture of tert-butyl (3R,4R)-4-(4-aminopyrazol-1-yl)-3-fluoropiperidine-1-carboxylate (X₃₆d) (310 mg, 1.090 mmol, 1 equiv), (4aS,5aR)-5,5-difluoro-5a-methyl-1H,4H,4aH,6H-cyclopropa[f]indazole-3-carboxylic acid (X₃₃ₑ) (298.6 mg, 1.308 mmol, 1.2 equiv) and HATU (621.8 mg, 1.635 mmol, 1.5 equiv) in DMF (3 mL) were added DIEA (422.7 mg, 3.270 mmol, 3 equiv) at room temperature. The resulting mixture was stirred at room temperature for 2 h. The resulting mixture was extracted with EtOAc. The combined organic layers were washed with water, dried over anhydrous Na₂SO₄. After filtration, the filtrate was concentrated under reduced pressure. The crude product was purified by reverse phase flash with the following conditions (H₂O/MeCN=1/1) to afford tert-butyl (3R,4R)-4-{4-[(4aS,5aR)-5,5-difluoro-5a-methyl-1H,4H,4aH,6H-cyclopropa[f]indazole-3-amido]pyrazol-1-yl}-3-fluoropiperidine-1-carboxylate (X₃₆d) (125 mg, 23.18%) as a white solid. MS (ESI) calc'd for (C₂₃H₂₉F₃N₆O₃) [M+1]⁺, 495.2; found, 495.4.

### Step 4: Synthesis of (4aS,5aR)-5,5-difluoro-N-{1-[(3R,4R)-3-fluoropiperidin-4-yl]pyrazol-4-yl}-4,5a-dimethyl-1H,4H,4aH,6H-cyclopropa[f]indazole-3-carboxamide (BBX₃₆)

To a stirred mixture of tert-butyl (3R,4R)-4-{4-[(4aS,5aR)-5,5-difluoro-5a-methyl-1H,4H,4aH,6H-cyclopropa[f]indazole-3-amido]pyrazol-1-yl}-3-fluoropiperidine-1-carboxylate (X₃₆d) (120 mg, 0.243 mmol, 1 equiv) in DCM (2 mL) was added HCl (gas) in 1,4-dioxane (2 mL, 4N) at room temperature. The mixture was stirred for 2 h at room temperature. The reaction was concentrated under reduced pressure, then lyophilized. This resulted in (4aS,5aR)-5,5-difluoro-N-{1-[(3R,4R)-3-fluoropiperidin-4-yl]pyrazol-4-yl}-4,5a-dimethyl-1H,4H,4aH,6H-cyclopropa[f]indazole-3-carboxamide (BBX₃₆) (104.3 mg, 67.73%) as a white solid. MS (ESI) calc'd for (C₁₈H₂₁F₃N₆O) [M+1]⁺, 395.2; found, 395.2. ¹H NMR (400 MHz, DMSO-d₆) δ 10.24 (s, 1H), 8.09 (s, 1H), 7.75 (s, 1H), 5.28-5.00 (m, 1H), 4.74-4.70 (m, 1H), 3.92-3.81 (m, 3H), 3.21-3.19 (m, 5H), 3.08-3.02 (m, 1H), 2.33-2.28 (m, 2H), 1.80-1.76 (m, 1H), 1.36 (s, 3H).

### Example 125. 6,6-dimethyl-3-{6-[(2R)-2-methylpiperazine-1-carbonyl]-1H-indol-2-yl}-4,5,6,7-tetrahydro-1H-indazole trihydrochloride (BBX₃₇)

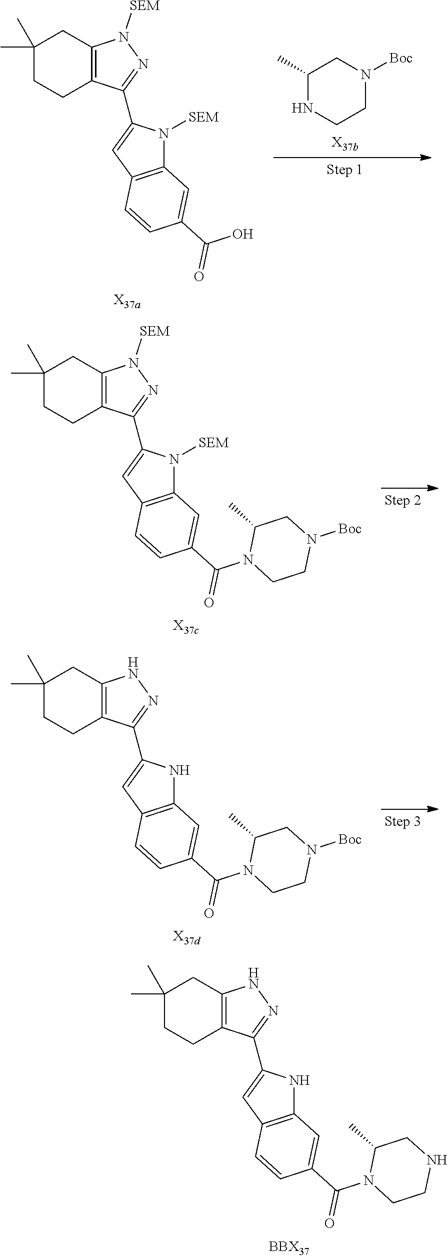 [SMILES: CC1(C)CCc2c(-c3cc4ccc(C(=O)O)cc4n3COCC[Si](C)(C)C)nn(COCC[Si](C)(C)C)c2C1 ; C[C@@H]1CN(C(=O)OC(C)(C)C)CCN1 ; C[C@@H]1CN(C(=O)OC(C)(C)C)CCN1C(=O)c1ccc2cc(-c3nn(COCC[Si](C)(C)C)c4c3CCC(C)(C)C4)n(COCC[Si](C)(C)C)c2c1 ; C[C@@H]1CN(C(=O)OC(C)(C)C)CCN1C(=O)c1ccc2cc(-c3n[nH]c4c3CCC(C)(C)C4)[nH]c2c1 ; C[C@@H]1CNCCN1C(=O)c1ccc2cc(-c3n[nH]c4c3CCC(C)(C)C4)[nH]c2c1]

### Step 1: Synthesis of tert-butyl (3R)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3-methylpiperazine-1-carboxylate (X_{37c})

To a solution of 2-(6,6-dimethyl-1-((2-(trimethylsilyl)ethoxy)methyl)-4,5,6,7-tetrahydro-1H-indazol-3-yl)-1-((2-(trimethylsilyl)ethoxy)methyl)-1H-indole-6-carboxylic acid (X₃₇ₐ) (0.569 g, 0.998 mmol, 1.0 eq) in DMF (10 ml, 0.1 M), DMAP (0.146 g, 1.195 mmol, 1.2 eq) and Et₃N (0.166 ml, 1.198 mmol, 1.2 eq) were added. EDC (0.287 g, 1.497 mmol, 1.5 eq) was added and the reaction mixture was stirred for 5 min. Then, (R)-tert-butyl 3-methylpiperazine-1-carboxylate (X₃₇b) (0.2 g, 1 mmol, 1.0 eq) was added and the reaction was stirred at room temperature until completion (2 days). The bases were quenched with 10% citric acid solution (until pH 5) and extracted with AcOEt. Organic layers were filtered, concentrated in vacuo, and purified by silica gel flash chromatography (Hex/AcOEt 10 to 50%) to provide tert-butyl (3R)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3-methylpiperazine-1-carboxylate (X_{37c}) (0.75, 99%). ¹H NMR (300 MHz, DMSO-d₆) δ 7.66-7.56 (m, 2H), 7.11 (dd, J=8.2, 1.0 Hz, 1H), 6.77 (s, 1H), 6.08 (s, 2H), 5.41 (s, 2H), 3.74 (s, 2H), 3.66-3.51 (m, 5H), 2.62 (t, J=5.9 Hz, 2H), 2.42 (s, 3H), 1.81-1.72 (m, 2H), 1.54 (t, J=5.7 Hz, 2H), 1.41 (s, 9H), 1.16 (dd, J=6.9, 2.0 Hz, 4H), 1.03 (s, 6H), 0.86 (t, J=8.0 Hz, 2H), 0.73-0.61 (m, 2H), −0.03 (s, 9H), −0.20 (s, 9H).

### Step 2: Synthesis of tert-butyl (3R)-4-[2-(6,6-dimethyl-4,5,6,7-tetrahydro-1H-indazol-3-yl)-1H-indole-6-carbonyl]-3-methylpiperazine-1-carboxylate (X₃₇ₐ)

To a solution of tert-butyl (3R)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3-methylpiperazine-1-carboxylate (X_{37c}) (0.74 g, 0.984 mmol, 1.0 eq) in DMF (6 ml, 0.18 M), were added TBAF in THE (5 ml, 4.8 mmol, 5 eq) concentrated in advance under reduced pressure, and ethylenediamine (0.987 ml, 14.764 mmol, 15 eq). The mixture was stirred at 90° C. overnight. After cooling, the mixture was diluted with H₂O and AcOEt and the organic layer was separated. Then, the aqueous layer was extracted twice with AcOEt. The combined organic layers were washed successively with water, 10% aqueous citric acid solution, and brine, dried over Na₂SO₄, filtered, and concentrated under reduced pressure. The residue was purified by silica gel flash chromatography (DCM/MeOH, 9.5:0.5 to 9:1) to afford tert-butyl (3R)-4-[2-(6,6-dimethyl-4,5,6,7-tetrahydro-1H-indazol-3-yl)-1H-indole-6-carbonyl]-3-methylpiperazine-1-carboxylate (X₃₇ₐ) (0.2 g, 41%). ¹H NMR (300 MHz, DMSO-d₆) δ 12.58 (s, 1H), 11.47 (s, 1H), 7.54 (d, J=8.2 Hz, 1H), 7.43 (s, 1H), 6.99 (d, J=7.4 Hz, 1H), 6.62 (s, 1H), 4.08-3.81 (m, 2H), 3.76 (d, J=11.9 Hz, 1H), 3.23-2.98 (m, 2H), 2.71-2.62 (m, 2H), 2.42 (s, 3H), 1.58 (t, J=5.9 Hz, 2H), 1.41 (s, 9H), 1.16 (d, J=6.5 Hz, 4H), 1.01 (s, 6H).

### Step 3: Synthesis of 6,6-dimethyl-3-{6-[(2R)-2-methylpiperazine-1-carbonyl]-1H-indol-2-yl}-4,5,6,7-tetrahydro-1H-indazole trihydrochloride (BBX₃₇)

tert-Butyl (3R)-4-[2-(6,6-dimethyl-4,5,6,7-tetrahydro-1H-indazol-3-yl)-1H-indole-6-carbonyl]-3-methylpiperazine-1-carboxylate (X₃₇ₐ) (0.2 g, 0.407 mmol, 1.0 eq) was dissolved in 2M HCl in Et₂O (1.22 ml, 2.44 mmol, 6 eq) at 0° C. After 2 h, the reaction was filtered and washed with fresh Et₂O to provide 6,6-dimethyl-3-{6-[(2R)-2-methylpiperazine-1-carbonyl]-1H-indol-2-yl}-4,5,6,7-tetrahydro-1H-indazole trihydrochloride (BBX₃₇) (0.201, 91%). LCMS method: LCMS2-036-10-60-95-6-1-25-UV, RT: 2.717 min, purity: 92.00% (254 nm), ESI(+) [M+H]⁺=392.0. ¹H NMR (300 MHz, DMSO-d₆) δ 11.52 (s, 1H), 9.49-9.39 (m, 1H), 8.99 (s, 1H), 7.56 (d, J=8.0 Hz, 1H), 7.49 (s, 1H), 7.05 (d, J=8.4 Hz, 1H), 6.64 (s, 1H), 3.46-3.23 (m, 3H), 3.17 (s, 2H), 2.69 (dd, J=13.2, 7.2 Hz, 2H), 2.42 (s, 3H), 1.58 (t, J=5.8 Hz, 2H), 1.36 (d, J=7.0 Hz, 3H), 1.09 (t, J=7.0 Hz, 1H), 1.01 (s, 6H).

### Example 126. 6,6-dimethyl-3-{6-[(2S)-2-methylpiperazine-1-carbonyl]-1H-indol-2-yl}-4,5,6,7-tetrahydro-1H-indazole trihydrochloride (BBX₃₈)

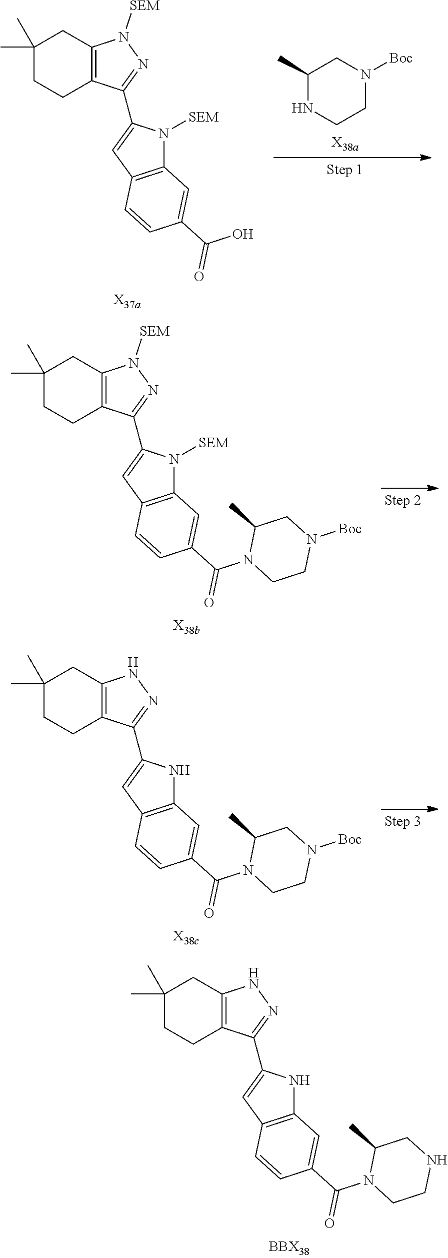 [SMILES: CC1(C)CCc2c(-c3cc4ccc(C(=O)O)cc4n3COCC[Si](C)(C)C)nn(COCC[Si](C)(C)C)c2C1 ; C[C@H]1CN(C(=O)OC(C)(C)C)CCN1 ; C[C@H]1CN(C(=O)OC(C)(C)C)CCN1C(=O)c1ccc2cc(-c3nn(COCC[Si](C)(C)C)c4c3CCC(C)(C)C4)n(COCC[Si](C)(C)C)c2c1 ; C[C@H]1CN(C(=O)OC(C)(C)C)CCN1C(=O)c1ccc2cc(-c3n[nH]c4c3CCC(C)(C)C4)[nH]c2c1 ; C[C@H]1CNCCN1C(=O)c1ccc2cc(-c3n[nH]c4c3CCC(C)(C)C4)[nH]c2c1]

### Step 1: Synthesis of tert-butyl (3S)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3-methylpiperazine-1-carboxylate (X_{38b})

To a solution of 2-(6,6-dimethyl-1-((2-(trimethylsilyl)ethoxy)methyl)-4,5,6,7-tetrahydro-1H-indazol-3-yl)-1-((2-(trimethylsilyl)ethoxy)methyl)-1H-indole-6-carboxylic acid (X₃₇ₐ) (0.569 g, 1 mmol, 1.0 eq) in DMF (10 ml, 0.1 M), DMAP (0.146 g, 1.2 mmol, 1.2 eq) and Et₃N (0.166 ml, 1.2 mmol, 1.2 eq) were added. EDC (0.287 g, 1.5 mmol, 1.5 eq) was added and reaction mixture was stirred for 5 min, then tert-butyl (S)-3-methylpiperazine-1-carboxylate (X₃₈ₐ) (0.2 g, 1 mmol, 1.0 eq) was added and the reaction was stirred at room temperature until completion (2 days). The bases were quenched with 10% citric acid solution (until pH 5) and extracted with AcOEt. The organic layers were filtered, concentrated in vacuo, and purified by silica gel flash chromatography (Hex/AcOEt 10 to 50%) to provide tert-butyl (3S)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3-methylpiperazine-1-carboxylate (X_{38b}) (0.75 g, 99%). ¹H NMR (300 MHz, DMSO-d₆) δ 7.68-7.56 (m, 2H), 7.11 (d, J=9.1 Hz, 1H), 6.77 (s, 1H), 6.08 (s, 2H), 5.41 (s, 2H), 3.76 (d, J=14.9 Hz, 2H), 3.59 (t, J=8.0 Hz, 3H), 2.63 (t, J=5.9 Hz, 2H), 2.42 (s, 3H), 1.54 (t, J=5.7 Hz, 2H), 1.41 (s, 9H), 1.16 (dd, J=6.9, 2.0 Hz, 4H), 1.03 (s, 6H), 0.87 (t, J=8.0 Hz, 2H), 0.77-0.61 (m, 2H), −0.03 (s, 9H), −0.20 (s, 9H).

### Step 2: Synthesis of tert-butyl (3S)-4-[2-(6,6-dimethyl-4,5,6,7-tetrahydro-1H-indazol-3-yl)-1H-indole-6-carbonyl]-3-methylpiperazine-1-carboxylate (X_{33c})

To a solution of tert-butyl (3S)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3-methylpiperazine-1-carboxylate (X_{33b}) (0.52 g, 0.691 mmol, 1.0 eq) in DMF (4 ml, 0.18 M) was added 1 M TBAF in THE (3.381 ml, 3.381 mmol, 5 eq), concentrated in advance under reduced pressure, and ethylenediamine (0.693 ml, 10.366 mmol, 15 eq). The mixture was stirred at 90° C. for overnight. After cooling, H₂O and AcOEt were added, and the organic layer was separated. Then, the aqueous layer was extracted twice with ethyl acetate. The combined organic layers were washed successively with H₂O, 10% aqueous citric acid solution and brine, dried over sodium sulfate, filtrated, and concentrated under reduced pressure. The residue was purified by silica gel flash chromatography (DCM/MeOH, 9.5:0.5 to 9:1) to provide tert-butyl (3S)-4-[2-(6,6-dimethyl-4,5,6,7-tetrahydro-1H-indazol-3-yl)-1H-indole-6-carbonyl]-3-methylpiperazine-1-carboxylate (X_{33c}) (0.22 g, 45%). ¹H NMR (300 MHz, DMSO-d₆) δ 12.58 (s, 1H), 11.47 (s, 1H), 7.54 (d, J=8.3 Hz, 1H), 7.44 (s, 1H), 6.99 (d, J=7.8 Hz, 1H), 6.62 (s, 1H), 3.98-3.86 (m, 2H), 3.76 (d, J=13.9 Hz, 2H), 3.24-3.01 (m, 2H), 2.68 (t, J=6.1 Hz, 2H), 2.42 (s, 3H), 1.59 (t, J=6.0 Hz, 2H), 1.42 (s, 9H), 1.16 (d, J=6.5 Hz, 4H), 1.02 (s, 6H).

### Step 3: Synthesis of 6,6-dimethyl-3-{6-[(2S)-2-methylpiperazine-1-carbonyl]-1H-indol-2-yl}-4,5,6,7-tetrahydro-1H-indazole trihydrochloride (BBX₃₈)

tert-Butyl (3S)-4-[2-(6,6-dimethyl-4,5,6,7-tetrahydro-1H-indazol-3-yl)-1H-indole-6-carbonyl]-3-methylpiperazine-1-carboxylate (X₃8c) (0.22 g, 0.447 mmol, 1.0 eq) was dissolved in HCl in Et₂O (1.4 ml, 2.68 mmol, 6 eq) at 0° C. After 2 h, the reaction was filtered and washed with fresh Et₂O to provide 6,6-dimethyl-3-{6-[(2S)-2-methylpiperazine-1-carbonyl]-1H-indol-2-yl}-4,5,6,7-tetrahydro-1H-indazole trihydrochloride (BBX₃₈) (0.229 g, 95% yield) as a grey powder. LCMS method: LCMS2-036-10-60-95-6-1-25-UV, RT=2.717 min, purity: 93.11% (254 nm), ESI(+) [M+H]⁺=392.27. ¹H NMR (300 MHz, DMSO-d₆) δ 11.59 (s, 1H), 9.66 (d, J=11.5 Hz, 1H), 9.22 (s, 1H), 7.58 (d, J=8.2 Hz, 1H), 7.50 (s, 1H), 7.07 (d, J=8.2 Hz, 1H), 6.71 (s, 1H), 4.08 (d, J=13.2 Hz, 2H), 3.47-3.27 (m, 3H), 3.27-3.06 (m, 2H), 2.70 (t, J=6.3 Hz, 2H), 2.44 (s, 3H), 1.59 (t, J=6.1 Hz, 2H), 1.37 (d, J=7.0 Hz, 3H), 1.09 (t, J=7.0 Hz, 1H), 1.01 (s, 6H).

### Example 127. 3-[6-(2,2-dimethylpiperazine-1-carbonyl)-1H-indol-2-yl]-6,6-dimethyl-4,5,6,7-tetrahydro-1H-indazole trihydrochloride (BBX₃₉)

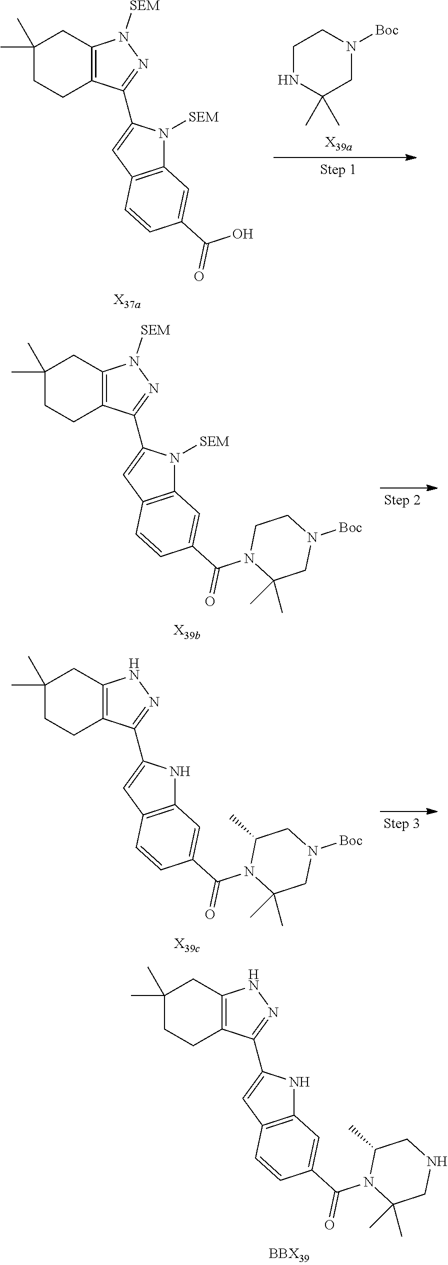 [SMILES: CC1(C)CCc2c(-c3cc4ccc(C(=O)O)cc4n3COCC[Si](C)(C)C)nn(COCC[Si](C)(C)C)c2C1 ; CC1(C)CN(C(=O)OC(C)(C)C)CCN1 ; CC1(C)CCc2c(-c3cc4ccc(C(=O)N5CCN(C(=O)OC(C)(C)C)CC5(C)C)cc4n3COCC[Si](C)(C)C)nn(COCC[Si](C)(C)C)c2C1 ; C[C@@H]1CN(C(=O)OC(C)(C)C)CC(C)(C)N1C(=O)c1ccc2cc(-c3n[nH]c4c3CCC(C)(C)C4)[nH]c2c1 ; C[C@@H]1CNCC(C)(C)N1C(=O)c1ccc2cc(-c3n[nH]c4c3CCC(C)(C)C4)[nH]c2c1]

### Step 1: Synthesis of tert-butyl 4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3,3-dimethylpiperazine-1-carboxylate (X₃₉n)

To a solution of 2-(6,6-dimethyl-1-((2-(trimethylsilyl)ethoxy)methyl)-4,5,6,7-tetrahydro-1H-indazol-3-yl)-1-((2-(trimethylsilyl)ethoxy)methyl)-1H-indole-6-carboxylic acid (X₃₇ₐ) (0.665 g, 1.17 mmol) in anhydrous DMF (11.7 ml, 0.1 M) were added DMAP (0.171 g, 1.4 mmol) and Et₃N (0.194 ml, 1.4 mmol), EDC (0.335 g, 1.75 mmol). The reaction mixture was stirred at room temperature for 5 min. The product was precipitated by adding ice. The suspension was stirred at room temperature for 15 min. Then, tert-butyl 3,3-dimethylpiperazine-1-carboxylate (X₃₉a) (0.25 g, 1.17 mmol) was added and the reaction was stirred at room temperature for 48 h. The bases were quenched with 10% citric acid solution (until pH 5) and extracted with EtOAc. The solvents were evaporated in vacuo. The residue was purified by silica gel flash chromatography eluted with hexane/EtOAc (10 to 50% of EtOAc) to provide tert-butyl 4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3,3-dimethylpiperazine-1-carboxylate (X₃₉n) (0.89 g, quantitative) as a yellow oil. LCMS: C₄₁H₆₇N₅O₅Si₂, desired mass=766.19, observed mass=767.5 [M+H]⁺. ¹H NMR (300 MHz, DMSO-d₆) δ 7.96 (s, 4H), 7.66-7.56 (m, 1H), 6.77 (d, J=10.0 Hz, 1H), 6.08 (s, 1H), 5.42 (d, J=7.1 Hz, 2H), 3.56 (dt, J=15.7, 6.9 Hz, 4H), 2.64 (d, J=13.8 Hz, 3H), 1.53 (d, J=12.1 Hz, 5H), 1.42 (s, 5H), 1.03 (d, J=2.3 Hz, 6H), 0.87 (t, J=8.0 Hz, 2H), 0.71 (dt, J=15.8, 7.5 Hz, 2H), −0.14-0.30 (m, 9H).

### Step 2: Synthesis of tert-butyl 4-[2-(6,6-dimethyl-4,5,6,7-tetrahydro-1H-indazol-3-yl)-1H-indole-6-carbonyl]-3,3-dimethylpiperazine-1-carboxylate (X_{39c})

To a solution of tert-butyl 4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-3,3-dimethylpiperazine-1-carboxylate (X₃₉n) (0.89 g, 1.16 mmol) in anhydrous DMF (6.45 ml, 0.18 M) were added TBAF in THE (5.81 ml, 5.81 mmol) concentrated in advance under reduced pressure, and ethylenediamine (1.17 ml, 17.4 mmol). The reaction mixture was stirred at 90° C. overnight. After cooling down, water and ethyl acetate were added, and the organic layer was separated. The aqueous layer was extracted with ethyl acetate. The combined organic layers were washed successively with water, 10% aqueous citric acid solution, saturated brine, and dried over sodium sulfate. Sodium sulfate was removed by filtration, and the solvents were evaporated in vacuo. The residue was purified by silica gel flash chromatography eluted with DCM/MeOH (5 to 10% of MeOH) to provide tert-butyl 4-[2-(6,6-dimethyl-4,5,6,7-tetrahydro-1H-indazol-3-yl)-1H-indole-6-carbonyl]-3,3-dimethylpiperazine-1-carboxylate (X_{39c}) (0.33 g, 56%) as a yellow oil. LCMS: C₂₉H₃₉N₅O₃, desired mass=505.66, observed mass=506.25 [M+H]⁺. ¹H NMR (300 MHz, DMSO-d₆) δ 12.57 (s, 1H), 11.44 (s, 1H), 7.52 (d, J=8.1 Hz, 1H), 7.41 (s, 1H), 6.99 (d, J=8.0 Hz, 1H), 6.61 (s, 1H), 3.53 (s, 2H), 3.45 (s, 2H), 3.38 (s, 2H), 2.68 (s, 2H), 2.42 (s, 2H), 1.58 (t, J=6.4 Hz, 3H), 1.50 (s, 6H), 1.43 (s, 9H), 1.01 (s, 6H).

### Step 3: Synthesis of 3-[6-(2,2-dimethylpiperazine-1-carbonyl)-1H-indol-2-yl]-6,6-dimethyl-4,5,6,7-tetrahydro-1H-indazole trihydrochloride (BBX₃₉)

tert-Butyl 4-[2-(6,6-dimethyl-4,5,6,7-tetrahydro-1H-indazol-3-yl)-1H-indole-6-carbonyl]-3,3-dimethylpiperazine-1-carboxylate (X_{39c}) (0.055 g, 0.109 mmol) was dissolved in HCl in Et₂O (0.33 ml, 0.653 mmol) at 0° C. After 2 h, the reaction was filtered, and the residue was washed with fresh Et₂O to provide 3-[6-(2,2-dimethylpiperazine-1-carbonyl)-1H-indol-2-yl]-6,6-dimethyl-4,5,6,7-tetrahydro-1H-indazole trihydrochloride (BBX₃₉) (0.04 g, 71% yield) as a light-yellow solid. LCMS: C₂₄H₃₄C₁₃N₅₀, desired mass=405.55, observed mass=406.0 [M+H]⁺, method: LCMS2-036-20-80-95-6-1-25-UV-BCM, RT=1.983 min, 95.72% purity (254 nm); ¹H NMR (300 MHz, DMSO-d₆) δ 11.46 (d, J=2.1 Hz, 1H), 8.21 (s, 1H), 7.58-7.42 (m, 2H), 7.06 (dd, J=8.2, 1.4 Hz, 1H), 6.61 (d, J=1.9 Hz, 1H), 3.30 (t, J=5.1 Hz, 3H), 2.87 (q, J=5.5, 4.1 Hz, 2H), 2.71 (d, J=16.8 Hz, 4H), 2.42 (s, 2H), 1.58 (t, J=6.3 Hz, 2H), 1.46 (s, 6H), 1.01 (s, 6H).

### Example 128. 6,6-dimethyl-3-{6-[(3S)-3-methylpiperazin-4-ium-1-carbonyl]-1H-indol-1-ium-2-yl}-4,5,6,7-tetrahydro-1H-indazol-1-ium trichloride (BBX₄₀)

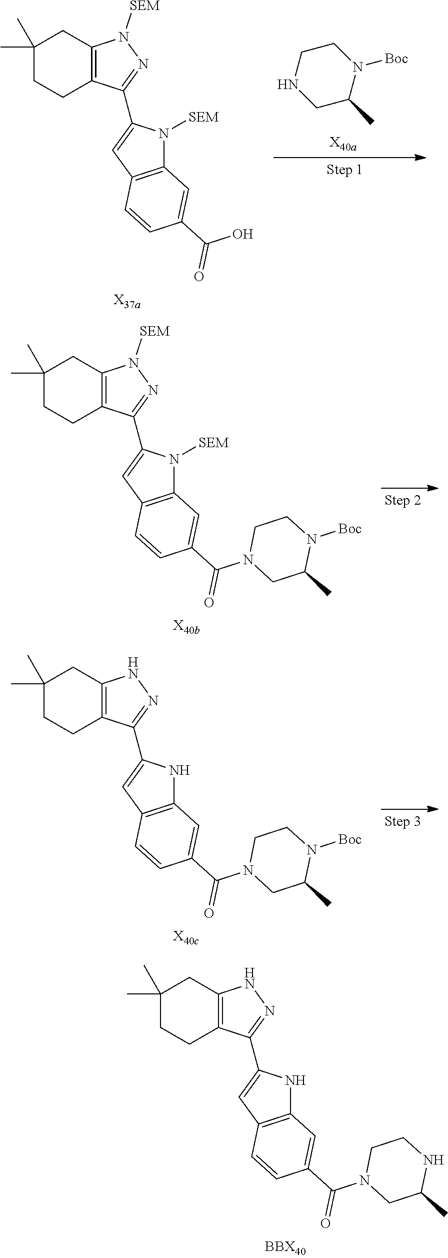

### Step 1: Synthesis of tert-butyl (2S)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2-methylpiperazine-1-carboxylate (X_{40b})

To a solution of 2-(6,6-dimethyl-1-((2-(trimethylsilyl)ethoxy)methyl)-4,5,6,7-tetrahydro-1H-indazol-3-yl)-1-((2-(trimethylsilyl)ethoxy)methyl)-1H-indole-6-carboxylic acid (X₃₇ₐ) (0.569 g, 0.998 mmol, 1.0 eq) in DMF (10 ml, 0.1 M), DMAP (0.146 g, 1.195 mmol, 1.2 eq) and Et₃N (0.166 ml, 1.2 mmol, 1.2 eq) were added. EDC (0.287 g, 1.5 mmol, 1.5 eq) was added and reaction mixture was stirred for 5 min, then tert-butyl (S)-2-methylpiperazine-1-carboxylate (X₄₀a) (0.2 g, 0.94 mmol, 1.0 eq) was added and the reaction was stirred at RT overnight. The bases were quenched with 10% citric acid solution (until pH 5) and extracted with AcOEt. The organic layers were washed with brine, dried with Na₂SO₄, filtered, concentrated in vacuo, and purified by silica gel flash chromatography (hexane/AcOEt, 10 to 50%) to provide tert-butyl (2S)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2-methylpiperazine-1-carboxylate (X_{40b}) (858 mg, 98%). ¹H NMR (300 MHz, DMSO-d₆) δ 7.63 (d, J=7.7 Hz, 2H), 7.14 (d, J=8.9 Hz, 1H), 6.77 (s, 1H), 6.08 (s, 2H), 5.41 (s, 2H), 4.12-4.21 (m, 1H), 3.77-3.68 (m, 2H), 3.59 (t, J=8.0 Hz, 2H), 2.62 (s, 3H), 1.54 (s, 2H), 1.41 (s, 9H), 1.09 (s, 4H), 1.03 (s, 6H), 0.87 (t, J=8.0 Hz, 2H), 0.75-0.61 (m, 2H), −0.03 (s, 9H), −0.19 (s, 9H).

### Step 2: Synthesis of tert-butyl (2S)-4-[2-(6,6-dimethyl-4,5,6,7-tetrahydro-1H-indazol-3-yl)-1H-indole-6-carbonyl]-2-methylpiperazine-1-carboxylate (X_{40c})

To a solution of tert-butyl (2S)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2-methylpiperazine-1-carboxylate (X_{40b}) (0.858 g, 0.9 mmol, 1.0 eq) in DMF (6 ml, 0.18 M) was added 1M TBAF in THF (5 ml, 4.9 mmol, 5 eq), concentrated in advance under reduced pressure, and ethylenediamine (1 ml, 14.7 mmol, 15 eq). The mixture was stirred at 90° C. overnight. After cooling, mixture was diluted with H₂O and AcOEt and the organic layer was separated. Then, the aqueous layer was extracted twice with AcOEt. The combined organic layers were washed successively with water, 10% aqueous citric acid solution and brine, dried over Na₂SO₄, filtered, and concentrated under reduced pressure. The residue was purified by silica gel flash chromatography (DCM/MeOH, 95:5 to 90:10) to provide tert-butyl (2S)-4-[2-(6,6-dimethyl-4,5,6,7-tetrahydro-1H-indazol-3-yl)-1H-indole-6-carbonyl]-2-methylpiperazine-1-carboxylate (X_{40c}) (0.484 g, 51%). ¹H NMR (300 MHz, DMSO-d₆) δ 12.58 (s, 1H), 11.48 (s, 1H), 7.54 (d, J=8.0 Hz, 1H), 7.46 (s, 1H), 7.01 (d, J=7.6 Hz, 1H), 6.62 (s, 1H), 4.28-4.01 (m, 2H), 3.84-3.66 (m, 1H), 3.23-2.89 (m, 3H), 2.68 (t, J=5.9 Hz, 2H), 2.42 (s, 3H), 1.58 (t, J=6.5 Hz, 2H), 1.41 (s, 9H), 1.08 (d, J=6.5 Hz, 3H), 1.01 (s, 6H).

### Step 3: Synthesis of 6,6-dimethyl-3-{6-[(3S)-3-methylpiperazin-4-ium-1-carbonyl]-1H-indol-1-ium-2-yl}-4,5,6,7-tetrahydro-1H-indazol-1-ium trichloride (BBX₄₀)

tert-Butyl (2S)-4-[2-(6,6-dimethyl-4,5,6,7-tetrahydro-1H-indazol-3-yl)-1H-indole-6-carbonyl]-2-methylpiperazine-1-carboxylate (X_{40c}) (0.25 g, 0.509 mmol, 1.0 eq) was dissolved in 2 M HCl in Et₂O (1.526 ml, 3.052 mmol, 6 eq) at 0° C. After 2 h, the reaction was filtered and washed with fresh Et₂O to provide 6,6-dimethyl-3-{6-[(3S)-3-methylpiperazin-4-ium-1-carbonyl]-1H-indol-1-ium-2-yl}-4,5,6,7-tetrahydro-1H-indazol-1-ium trichloride (BBX₄₀) (0.267 g, 99%) as light brown powder. LCMS method: LCMS2-036-10-60-95-6-1-25-UV, RT: 2.703 min, purity: 94.29% (254 nm), ESI(+) [M+H]⁺=392.03. ¹H NMR (300 MHz, DMSO-d₆) δ 11.53 (s, 1H), 9.39 (s, 1H), 9.16 (s, 1H), 7.56 (d, J=8.0 Hz, 1H), 7.52 (s, 1H), 7.08 (d, J=8.3 Hz, 1H), 6.65 (s, 1H), 3.46-3.21 (m, 3H), 3.17 (s, 1H), 3.15-2.99 (m, 2H), 2.68 (t, J=5.4 Hz, 2H), 2.42 (s, 3H), 1.58 (t, J=6.3 Hz, 2H), 1.23 (d, J=5.5 Hz, 3H), 1.01 (s, 6H).

### Example 129. 6,6-dimethyl-3-{6-[(3R)-3-methylpiperazine-1-carbonyl]-1H-indol-2-yl}-4,5,6,7-tetrahydro-1H-indazole (BBX₄₁)

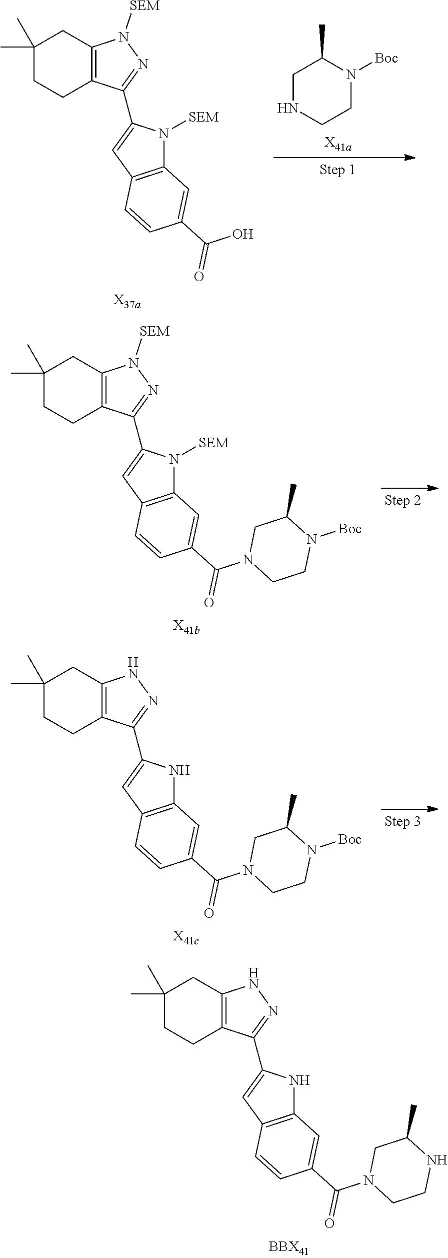 [SMILES: CC1(C)CCc2c(-c3cc4ccc(C(=O)O)cc4n3COCC[Si](C)(C)C)nn(COCC[Si](C)(C)C)c2C1 ; C[C@@H]1CNCCN1C(=O)OC(C)(C)C ; C[C@@H]1CN(C(=O)c2ccc3cc(-c4nn(COCC[Si](C)(C)C)c5c4CCC(C)(C)C5)n(COCC[Si](C)(C)C)c3c2)CCN1C(=O)OC(C)(C)C ; C[C@@H]1CN(C(=O)c2ccc3cc(-c4n[nH]c5c4CCC(C)(C)C5)[nH]c3c2)CCN1C(=O)OC(C)(C)C ; C[C@@H]1CN(C(=O)c2ccc3cc(-c4n[nH]c5c4CCC(C)(C)C5)[nH]c3c2)CCN1]

### Step 1: Synthesis of tert-butyl (2R)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2-methylpiperazine-1-carboxylate (X_{41b})

To solution of 2-(6,6-dimethyl-1-((2-(trimethylsilyl)ethoxy)methyl)-4,5,6,7-tetrahydro-1H-indazol-3-yl)-1-((2-(trimethylsilyl)ethoxy)methyl)-1H-indole-6-carboxylic acid (X₃₇ₐ) (5.0 g, 8.774 mmol, 1.0 eq) in anhydrous dimethylformamide (87.74 ml, 0.1 M), were added 4-(dimethylamino)pyridine (1.286 g, 10.526 mmol, 1.2 eq), triethylamine (1.459 ml, 10.525 mmol, 1.2 eq) and EDC hydrochloride (2.656 g, 13.162 mmol, 1.5 eq). After 5 min, (R)-1-N-Boc-2-methylpiperazine (X₄₁a) (1.845 g, 9.212 mmol, 1.05 eq) was added to this solution at room temperature. Reaction mixture was allowed to stir at room temperature for 16 h under argon. Ice cold water (400 ml) was added to the mixture and extracted with ethyl acetate (3×200 mL). The combined organic layers were washed with 5% aq. KHSO₄ (300 ml×2), brine (300 mL×2), dried over Na₂SO₄, filtered and concentrated under reduced pressure to give crude which was purified by flash chromatography (hexane/ethyl acetate, gradient 10-50%) to give tert-butyl (2R)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2-methylpiperazine-1-carboxylate (X_{41b}) (5.7 g, 86%) as a colourless viscous oil. ESI(+)[M+H]⁺ not observed. ¹H NMR (300 MHz, DMSO-d₆) δ 7.67-7.58 (m, 2H), 7.14 (d, J=8.5 Hz, 1H), 6.77 (s, 1H), 6.08 (s, 2H), 5.41 (s, 2H), 4.17 (s, 2H), 3.72 (s, 1H), 3.65-3.53 (m, 2H), 3.41-3.31 (m, 1H), 3.16-3.01 (m, 2H), 2.63 (s, 2H), 2.29-2.18 (m, 1H), 1.54 (t, J=6.1 Hz, 2H), 1.41 (s, 9H), 1.24 (s, 1H), 1.18-1.06 (m, 4H), 1.03 (s, 6H), 0.92-0.81 (m, 2H), 0.68 (dd, J=8.6, 7.4 Hz, 2H), −0.3 (s, 9H), −0.20 (s, 9H).

### Step 2: Synthesis of tert-butyl (2R)-4-[2-(6,6-dimethyl-4,5,6,7-tetrahydro-1H-indazol-3-yl)-1H-indole-6-carbonyl]-2-methylpiperazine-1-carboxylate (X_{41c})

To a solution of tert-butyl (2R)-4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2-methylpiperazine-1-carboxylate (X_{41b}) (5.6 g, 7.445 mmol, 1.0 eq), in anhydrous dimethylformamide (41.36 ml, 0.18 M) was added to TBAF (36.407 ml, 36.407 mmol, 4.89 eq) obtained after concentration of 1M solution in THF under argon atmosphere. Ethylenediamine (7.466 ml, 111.679 mmol, 15.0 eq) was added. Reaction mixture was allowed to stir at 90° C. overnight. After cooling, mixture was diluted with water (300 ml) and extracted with ethyl acetate. The combined organic layers were washed successively with water (200 mL×2), and brine (200 mL×2), dried over Na₂SO₄, filtered and concentrated under reduced pressure. The residue was purified by silica gel chromatography twice (DCM:MeOH, gradient 0.1%-0.5% MeOH in DCM) to afford tert-butyl (2R)-4-[2-(6,6-dimethyl-4,5,6,7-tetrahydro-1H-indazol-3-yl)-1H-indole-6-carbonyl]-2-methylpiperazine-1-carboxylate (X_{41c}) (2.45 g, 67%) as foamy solid. ESI(+) [M+H]⁺=492.2. ¹H NMR (300 MHz, DMSO-d₆) δ 12.58 (s, 1H), 11.53 (s, 1H), 7.54 (d, J=8.2 Hz, 1H), 7.46 (s, 1H), 7.01 (d, J=7.8 Hz, 1H), 6.62 (s, 1H), 4.20-4.11 (m, 2H), 3.81-3.66 (m, 1H), 3.23-2.86 (m, 2H), 2.68 (t, J=6.1 Hz, 2H), 2.42 (s, 2H), 1.58 (t, J=6.1 Hz, 2H), 1.41 (s, 9H), 1.28-1.18 (m, 2H), 1.08 (d, J=6.8 Hz, 3H), 1.01 (s, 6H).

### Step 3: Synthesis of 6,6-dimethyl-3-{6-[(3R)-3-methylpiperazine-1-carbonyl]-1H-indol-2-yl}-4,5,6,7-tetrahydro-1H-indazole (BBX₄₁)

tert-Butyl (2R)-4-[2-(6,6-dimethyl-4,5,6,7-tetrahydro-1H-indazol-3-yl)-1H-indole-6-carbonyl]-2-methylpiperazine-1-carboxylate (X_{41b}) (2.4 g, 4.882 mmol, 1.0 eq) was dissolved in 4 M HCl in dioxane (61.02 ml, 244.08 mmol, 50.0 eq) at 0° C. Reaction mixture was stirred at room temperature for 2 h. Volatiles were removed under reduced pressure. Obtained residue was triturated with diethyl ether (70 mL×3) to give 6,6-dimethyl-3-{6-[(3R)-3-methylpiperazine-1-carbonyl]-1H-indol-2-yl}-4,5,6,7-tetrahydro-1H-indazole (BBX₄₁) (1.98 g, 95%) as HCl salt (mono) in the form of light pink dry solid. LCMS (254 nm): RT=1.807 min, 97.57% purity, ESI(+)[M+H]⁺=392.30. ¹H NMR (300 MHz, DMSO-d₆) δ 11.54 (s, 1H), 9.67-9.51 (m, 1H), 9.51-9.30 (m, 1H), 7.57 (d, J=8.2 Hz, 1H), 7.52 (s, 1H), 7.09 (dd, J=8.1, 1.2 Hz, 1H), 6.68 (s, 1H), 4.24-4.12 (m, 2H), 3.46-3.22 (m, 2H), 3.2-2.96 (m, 2H), 2.69 (t, J=5.9 Hz, 2H), 2.43 (s, 2H), 1.59 (t, J=6.1 Hz, 2H), 1.25 (d, J=6.1 Hz, 3H), 1.01 (s, 6H).

### Example 130. 3-[6-(3,3-dimethylpiperazine-1-carbonyl)-1H-indol-2-yl]-6,6-dimethyl-4,5,6,7-tetrahydro-1H-indazole (BBX₄₂)

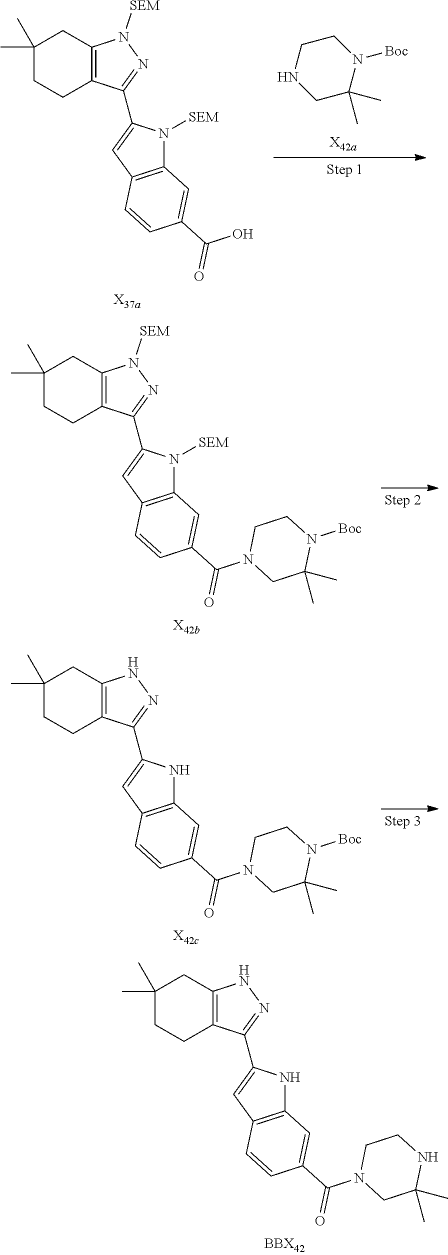

### Step 1: Synthesis of tert-Butyl 4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2,2-dimethylpiperazine-1-carboxylate (X₄₂b)

To a solution of 2-(6,6-dimethyl-1-((2-(trimethylsilyl)ethoxy)methyl)-4,5,6,7-tetrahydro-1H-indazol-3-yl)-1-((2-(trimethylsilyl)ethoxy)methyl)-1H-indole-6-carboxylic acid (X₃₇ₐ) (0.532 g, 0.933 mmol, 1.0 eq) in DMF (10 ml, 0.1 M), DMAP (0.137 g, 1.12 mmol, 1.2 eq) and Et₃N (0.155 ml, 1.2 mmol, 1.2 eq) were added. EDC (0.27 g, 1.4 mmol, 1.5 eq) was added and the reaction mixture was stirred for 5 min. Then, tert-butyl 2,2-dimethylpiperazine-1-carboxylate (X₄₂a) (0.2 g, 0.94 mmol, 1.0 eq) was added and the reaction was stirred at room temperature overnight. The bases were quenched with 10% citric acid solution (until pH 5) and extracted with AcOEt. The organic layers were washed with brine, dried with Na₂SO₄, filtered, concentrated in vacuo, and purified by silica gel flash chromatography (hexane/AcOEt, 10 to 50%) to provide tert-butyl 4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2,2-dimethylpiperazine-1-carboxylate (X₄₂b) (800 mg, 93%). ¹H NMR (300 MHz, DMSO-d₆) δ 7.70-7.55 (m, 2H), 7.15 (d, J=8.6 Hz, 1H), 6.77 (s, 1H), 6.08 (s, 2H), 5.40 (s, 2H), 3.67-3.45 (m, 6H), 2.62 (s, 2H), 1.54 (s, 2H), 1.41 (s, 9H), 1.02 (s, 6H), 0.86 (t, J=8.0 Hz, 2H), 0.66 (t, J=7.9 Hz, 2H), 0.04 (s, 9H), 0.20 (s, 9H).

### Step 2: Synthesis of tert-Butyl 4-[2-(6,6-dimethyl-4,5,6,7-tetrahydro-1H-indazol-3-yl)-1H-indole-6-carbonyl]-2,2-dimethylpiperazine-1-carboxylate (X_{42c})

To a solution of tert-butyl 4-[2-(6,6-dimethyl-1-{[2-(trimethylsilyl)ethoxy]methyl}-4,5,6,7-tetrahydro-1H-indazol-3-yl)-1-{[2-(trimethylsilyl)ethoxy]methyl}-1H-indole-6-carbonyl]-2,2-dimethylpiperazine-1-carboxylate (X₄₂b) (0.8 g, 0.9 mmol, 1.0 eq) in DMF (5 ml, 0.18 M) was added to TBAF in THE (4.5 ml, 4.4 mmol, 5 eq) concentrated in advance under reduced pressure, and ethylenediamine (0.9 ml, 13.3 mmol, 15 eq). The mixture was stirred at 90° C. overnight. After cooling, water and ethyl acetate were added, and the organic layer was separated. Then, the aqueous layer was extracted twice with AcOEt. The combined organic layers were washed successively with water, 10% aqueous citric acid solution and saturated brine and dried over sodium sulfate. Sodium sulfate was removed by filtration, the filtrate was concentrated under reduced pressure. The residue was purified by silica gel chromatography (DCM/MeOH, 5 to 10%) to provide tert-butyl 4-[2-(6,6-dimethyl-4,5,6,7-tetrahydro-1H-indazol-3-yl)-1H-indole-6-carbonyl]-2,2-dimethylpiperazine-1-carboxylate (X_{42c}) (155 mg, 33%). ¹H NMR (300 MHz, DMSO-d₆) δ 12.58 (s, 1H), 11.47 (s, 1H), 7.54 (d, J=8.1 Hz, 1H), 7.46 (s, 1H), 7.03 (d, J=8.0 Hz, 1H), 6.62 (s, 1H), 3.55 (s, 3H), 2.70 (d, J=16.0 Hz, 2H), 1.58 (s, 2H), 1.41 (s, 9H), 1.24 (s, 6H), 1.01 (s, 6H).

### Step 3: Synthesis of 3-[6-(3,3-dimethylpiperazine-1-carbonyl)-1H-indol-2-yl]-6,6-dimethyl-4,5,6,7-tetrahydro-1H-indazole (BBX₄₂)

tert-Butyl 4-[2-(6,6-dimethyl-4,5,6,7-tetrahydro-1H-indazol-3-yl)-1H-indole-6-carbonyl]-2,2-dimethylpiperazine-1-carboxylate (X_{42c}) (155 mg, 0.3 mmol, 1.0 eq) was dissolved in 2 M HCl in Et₂O (1 ml, 0.3 M) at room temperature. Reaction was completed after 2 h, product was filtered off and washed with fresh Et₂O to provide 3-[6-(3,3-dimethylpiperazine-1-carbonyl)-1H-indol-2-yl]-6,6-dimethyl-4,5,6,7-tetrahydro-1H-indazole (BBX₄₂) (179 mg, 94%) as a pink powder. ESI(+) [M+H]⁺=505.80. ¹H NMR (300 MHz, DMSO-d₆) δ 11.51 (s, 1H), 9.21 (s, 2H), 7.57 (d, J=8.1 Hz, 1H), 7.50 (s, 1H), 7.08 (d, J=9.4 Hz, 1H), 6.64 (s, 1H), 3.19 (d, J=9.7 Hz, 3H), 2.68 (s, 2H), 2.42 (s, 4H), 1.59 (s, 2H), 1.32 (s, 6H), 1.01 (s, 6H).

### Example 131. 3-[5-fluoro-6-(piperazine-1-carbonyl)-1H-indol-2-yl]-6,6-dimethyl-4,5,6,7-tetrahydro-1H-indazole (BBX₄₃)

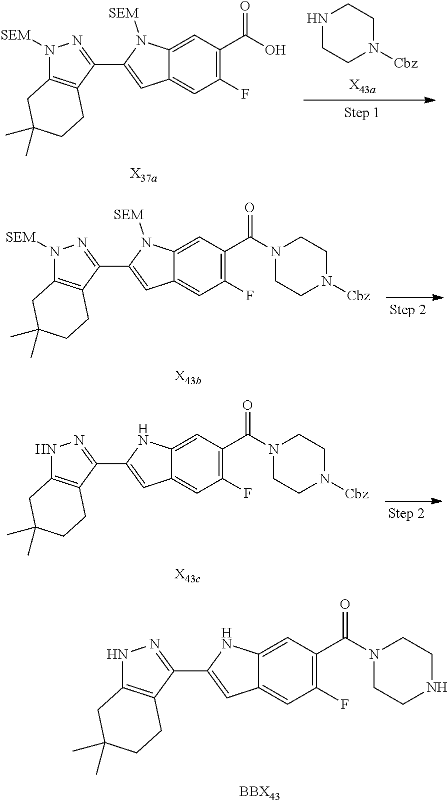

### Step 1: Synthesis of benzyl 4-[2-(6,6-dimethyl-1-{[2-(trimethylsilyl)ethoxy]methyl}-4,5,6,7-tetrahydro-1H-indazol-3-yl)-5-fluoro-1-{[2-(trimethylsilyl)ethoxy]methyl}-1H-indole-6-carbonyl]piperazine-1-carboxylate (X_{43b})

To a solution of 2-(6,6-dimethyl-1-{[2-(trimethylsilyl)ethoxy]methyl}-4,5,6,7-tetrahydro-1H-indazol-3-yl)-5-fluoro-1-{[2-(trimethylsilyl)ethoxy]methyl}-1H-indole-6-carboxylic acid (X₃₇ₐ) (P-43) (0.432 g, 0.735 mmol, 1.0 eq) in anhydrous dimethylformamide (7.35 ml, 0.1 M) were added DMAP (0.108 g, 0.884 mmol, 1.203 eq) and Et₃N (0.122 ml, 0.88 mmol, 1.198 eq). EDC (0.211 g, 1.101 mmol, 1.498 eq) was added and reaction mixture was stirred for 5 min. Then, benzyl 1-piperazinecarboxylate (X₄₃a) (0.142 ml, 0.736 mmol, 1.002 eq) was added and the reaction was stirred at room temperature for 2 days. The bases were quenched with 10% citric acid solution to get pH-5 and extracted three times with 25 mL of AcOEt. The combined organic layers were washed with water (2×30 mL) and then brine. The solution was dried on sodium sulfate, filtered, and concentrated under reduced pressure. The residue was purified by silica gel chromatography (Hexane/AcOEt, 7/3) affording benzyl 4-[2-(6,6-dimethyl-1-{[2-(trimethylsilyl)ethoxy]methyl}-4,5,6,7-tetrahydro-1H-indazol-3-yl)-5-fluoro-1-{[2-(trimethylsilyl)ethoxy]methyl}-1H-indole-6-carbonyl]piperazine-1-carboxylate (X_{43b}) (340 mg, 59%). ¹H NMR (300 MHz, Chloroform-d) δ 7.38 (s, 7H), 6.68 (s, 1H), 5.44 (s, 2H), 5.18 (s, 3H), 3.64 (dd, J=10.5, 5.8 Hz, 5H), 3.47-3.39 (m, 3H), 2.67 (t, J=6.2 Hz, 2H), 2.51 (s, 2H), 2.07 (s, 2H), 1.61 (t, J=6.2 Hz, 4H), 1.09 (s, 7H), 0.12 (d, J=3.2 Hz, 9H).

### Step 2: Synthesis of benzyl 4-[2-(6,6-dimethyl-4,5,6,7-tetrahydro-1H-indazol-3-yl)-5-fluoro-1H-indole-6-carbonyl] piperazine-1-carboxylate (X_{43c})

To a solution of benzyl 4-[2-(6,6-dimethyl-1-{[2-(trimethylsilyl)ethoxy]methyl}-4,5,6,7-tetrahydro-1H-indazol-3-yl)-5-fluoro-1-{[2-(trimethylsilyl)ethoxy]methyl}-1H-indole-6-carbonyl]piperazine-1-carboxylate (X_{43b}) (0.757 g, 0.958 mmol, 1.0 eq) in anhydrous dimethylformamide (5.32 ml, 0.18 M) was added TBAF in THE (5 ml, 5 mmol, 4.89 eq), concentrated in advance under reduced pressure, and ethylenediamine (0.961 ml, 14.4 mmol, 15.0 eq). The mixture was stirred at 90° C. for 4 hours. After cooling, water and ethyl acetate were added, and the organic layer was separated. Then, the aqueous layer was extracted with ethyl acetate (3×25 mL). The combined organic layers were washed successively with water (2×25 mL), 10% aqueous citric acid solution (25 mL) and saturated brine and dried over sodium sulfate.

Sodium sulfate was removed by filtration, and the filtrate was concentrated under reduced pressure. The residue was purified by silica gel chromatography (Hexane/AcOEt, 0 to 100%) to give benzyl 4-[2-(6,6-dimethyl-4,5,6,7-tetrahydro-1H-indazol-3-yl)-5-fluoro-1H-indole-6-carbonyl] piperazine-1-carboxylate (X_{43c}) (200 mg, 39%). ¹HNMR (300 MHz, DMSO-d₆) δ 12.62 (s, 1H), 11.54 (s, 1H), 7.49-7.27 (m, 7H), 6.62 (s, 1H), 5.11 (s, 2H), 3.68 (s, 2H), 3.51 (s, 2H), 2.79-2.57 (m, 3H), 2.42 (s, 2H), 1.91 (s, 1H), 1.58 (t, J=6.3 Hz, 2H), 1.24 (s, 3H), 1.01 (s, 6H).

### Step 3: Synthesis of 3-[5-fluoro-6-(piperazine-1-carbonyl)-1H-indol-2-yl]-6,6-dimethyl-4,5,6,7-tetrahydro-1H-indazole (BBX₄₃)

A solution of benzyl 4-[2-(6,6-dimethyl-4,5,6,7-tetrahydro-1H-indazol-3-yl)-5-fluoro-1H-indole-6-carbonyl] piperazine-1-carboxylate (X_{43c}) (0.200 g, 0.378 mmol, 1.0 eq) in methanol (9.44 ml, 0.04 M) with 10 wt % Palladium on carbon 60-65% wet (0.02 g, 0.2 mmol, 0.5 eq) in one portion and stir the mixture under 1 atmosphere of hydrogen gas for 24 h. No conversion was observed so 15% by weight of Pd(OH)₂ was added and the reaction was stirred for 3 days. No significant conversion was observed so the reaction was diluted in 80 mL of MeOH and 70% by weight of Pd/C was added then the reaction was stirred for another one hour. It was observed the complete conversion of benzyl 4-[2-(6,6-dimethyl-4,5,6,7-tetrahydro-1H-indazol-3-yl)-5-fluoro-1H-indole-6-carbonyl] piperazine-1-carboxylate (X_{43c}) but only 20% into the desired product and the rest into mono, di and trimethylated side-products. The solution was filtered through a celite pad and concentrated. The residue was purified by preparative HPLC to give 3-[5-fluoro-6-(piperazine-1-carbonyl)-1H-indol-2-yl]-6,6-dimethyl-4,5,6,7-tetrahydro-1H-indazole (BBX₄₃) (3.2 mg, 2%) as a yellow solid. LCMS method: ESI(+) [M+H]⁺=395.48. ¹H NMR (300 MHz, Acetonitrile-d₃) δ 10.09 (s, 1H), 8.15 (s, 2H), 7.41 (d, J=5.6 Hz, 1H), 7.33 (d, J=10.8 Hz, 1H), 6.71 (d, J=2.4 Hz, 1H), 3.87-3.77 (m, 2H), 3.50-3.40 (m, 2H), 3.10-2.99 (m, 2H), 2.96-2.84 (m, 3H), 2.75 (t, J=6.4 Hz, 3H), 2.11 (s, 1H), 1.65 (t, J=6.4 Hz, 2H), 1.29 (s, 2H), 1.05 (s, 6H).

### Example 132. N-(2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)pivalamide (X₃₇ₐ-1)

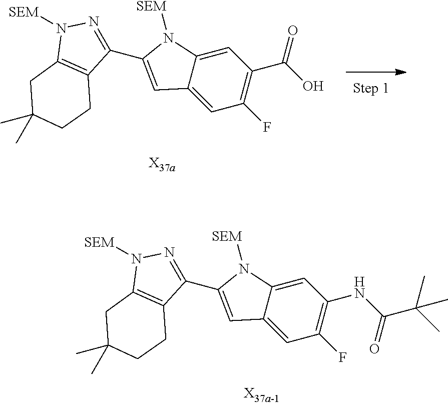 [SMILES: CC1(C)CCc2c(-c3cc4cc(F)c(C(=O)O)cc4n3COCC[Si](C)(C)C)nn(COCC[Si](C)(C)C)c2C1 ; CC1(C)CCc2c(-c3cc4cc(F)c(NC(=O)C(C)(C)C)cc4n3COCC[Si](C)(C)C)nn(COCC[Si](C)(C)C)c2C1]

### Step 1: Synthesis of N-[2-(6,6-dimethyl-1-{[2-(trimethylsilyl)ethoxy]methyl}-4,5,6,7-tetrahydro-1H-indazol-3-yl)-5-fluoro-1-{[2-(trimethylsilyl)ethoxy]methyl}-1H-indol-6-yl]-2,2-dimethylpropanamide

To a mixture of 2-(6,6-dimethyl-1-{[2-(trimethylsilyl)ethoxy]methyl}-4,5,6,7-tetrahydro-1H-indazol-3-yl)-5-fluoro-1-{[2-(trimethylsilyl)ethoxy]methyl}-1H-indole-6-carboxylic acid (1.15 g, 1.96 mmol) in toluene (25 ml) was added DPPA (0.7 ml, 3 mmol), triethylamine (0.7 ml, 5 mmol) and t-BuOH (0.73 ml, 7.6 mmol). The mixture was stirred under nitrogen at 70° C. for 30 min then at 100° C. for 4 h. After cooling, water was added to the reaction mixture, and the organic layer was separated. Then, the organic layer was washed successively with water and saturated brine, dried over magnesium sulfate, filtrated, and concentrated under reduced pressure. The crude material was purified by silica gel chromatography. Diphenyl phosphonate was contaminating the product that was precipitated from hexane to provide 0.778 g (60% yield) of the title compound. LCMS: C₃₄H₅₅FN₄O₃Si₂ requires: 642.4, found: m/z=541.4, method: LCMS2-036-95-95-95-6-1-40-UV, RT=1.903 min, 99.33% purity (254 nm); ¹H NMR (300 MHz, DMSO-d₆) δ 8.80 (s, 1H), 7.77 (d, J=5.6 Hz, 1H), 7.35 (d, J=11.0 Hz, 2H), 6.65 (s, 1H), 5.99 (s, 2H), 5.40 (s, 2H), 3.58 (t, J=8.0 Hz, 2H), 3.28 (d, J=8.3 Hz, 2H), 2.60 (t, J=5.6 Hz, 2H), 1.53 (t, J=6.0 Hz, 2H), 1.48 (s, 9H), 1.02 (s, 6H), 0.86 (t, J=8.0 Hz, 2H), 0.76-0.63 (m, 2H), −0.03 (s, 9H), −0.19 (s, 9H).

### Example 133. 2-[(4aS,5aR)-5,5-Difluoro-5a-methyl-1-(oxan-2-yl)-1H,4H,4aH,5H,5aH,6H-cyclopropa[f]indazol-3-yl]-5-fluoro-1H-indole-6-carboxylic acid (BBX₄₄)

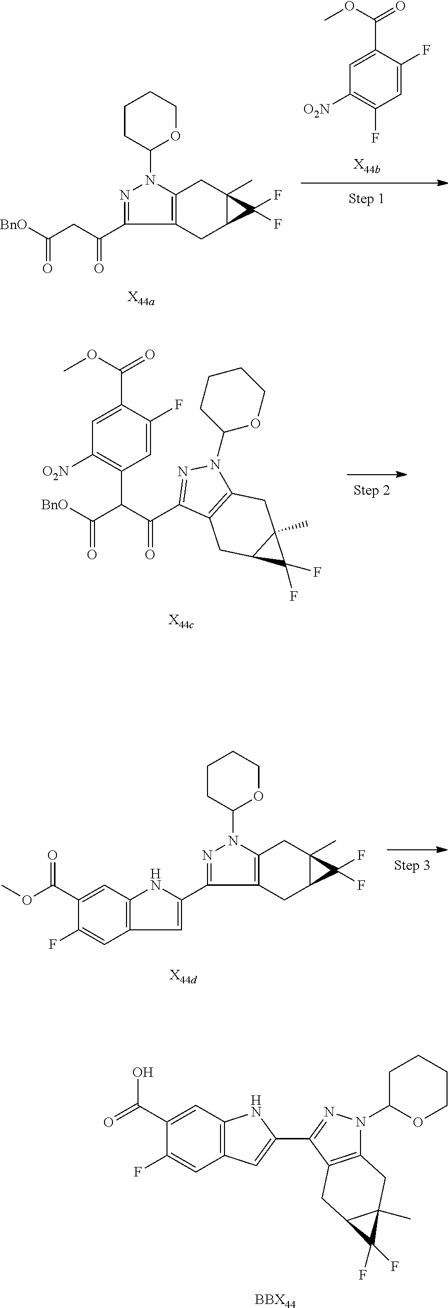

### Step 1: Synthesis of methyl 4-{1-[(4aS,5aR)-5,5-difluoro-5a-methyl-1-(oxan-2-yl)-1H,4H,4aH,5H,5aH, 6H-cyclopropa[f]indazol-3-yl]-3-(benzyloxy)-1,3-dioxopropan-2-yl}-2-fluoro-5-nitrobenzoate (X₄₄c)

To a solution of benzyl 3-[(4aS,5aR)-5,5-difluoro-5a-methyl-1-(oxan-2-yl)-1H,4H,4aH,5H,5aH,6H-cyclopropa[f]indazol-3-yl]-3-oxopropanoate (X₄₄a) (0.91 g, 2.047 mmol) in anhydrous dimethyl sulfoxide (8.19 mL, 0.25 M) were successively added methyl 2,4-difluoro-5-nitrobenzoate (X₄₄b) (0.533 g, 2.457 mmol) and potassium carbonate anhydrous (0.566 g, 4.095 mmol). The mixture was stirred at 90° C. for 2 h. The reaction solution was cooled to room temperature and poured into 30 ml of cold water. Then the mixture was extracted with ethyl acetate (30 mL×4), and combined organic layers were washed successively with water (20 mL×2) and saturated brine (20 mL×2). The obtained organic layer was dried over anhydrous sodium sulfate, and the filtrate was concentrated under reduced pressure. The residue was purified by flash chromatography (Hexane and 5% EtOAc in DCM mixture, gradient 5 to 20%) to provide methyl 4-{1-[(4aS,5aR)-5,5-difluoro-5a-methyl-1-(oxan-2-yl)-1H,4H,4aH,5H,5aH, 6H-cyclopropa[f]indazol-3-yl]-3-(benzyloxy)-1,3-dioxopropan-2-yl}-2-fluoro-5-nitrobenzoate (X_{44c}) (0.66 g, 45%) as a white solid. LCMS: C₃₂H₃₀F₃N₃O₅, desired mass=641.2 observed mass=642.1 [M+H]⁺. ¹H NMR (300 MHz, DMSO-d6) δ 8.60-8.48 (m, 1H), 7.46-7.40 (m, 1H), 7.40-7.20 (m, 4H), 6.52-6.42 (m, 1H), 5.54-5.40 (m, 1H), 5.25 (dd, J=7.3, 4.3 Hz, 2H), 3.95-3.85 (m, 3H), 3.75 (d, J=12.4 Hz, 1H), 3.62 (t, J=10.7 Hz, 1H), 3.48 (s, 1H), 3.00 (s, 3H), 2.25 (d, J=3.8 Hz, 1H), 1.99 (s, 2H), 1.80 (d, J=15.6 Hz, 2H), 1.52 (s, 3H), 1.39-1.27 (m, 3H), 1.25 (d, J=7.0 Hz, 1H).

### Step 2: Synthesis of methyl 2-[(4aS,5aR)-5,5-difluoro-5a-methyl-1-(oxan-2-yl)-1H,4H,4aH,5H,5aH,6H-cyclopropa[f]indazol-3-yl]-5-fluoro-1H-indole-6-carboxylate (X₄₄a)

To a solution of methyl 4-{1-[(4aS,5aR)-5,5-difluoro-5a-methyl-1-(oxan-2-yl)-1H,4H,4aH, 5H,5aH,6H-cyclopropa[f]indazol-3-yl]-3-(benzyloxy)-1,3-dioxopropan-2-yl}-2-fluoro-5-nitrobenzoate (X_{44c}) (0.65 g, 0.912 mmol) in anhydrous toluene (9.12 ml, 0.1 M) and ethanol (9.12 ml, 0.1 M) was added 10 wt % palladium on carbon 60-65% wet (0.102 g, 0.957 mmol). The reaction mixture was purged trice with vacuum/argon followed by vacuum/hydrogen trice and stirred at room temperature overnight. The reaction mixture was filtered through a celite pad. The filter cake was washed with methanol (10 mL×2). The filtrate was evaporated to dryness. The crude was purified by silica gel flash chromatography using hexane/EtOAc to give methyl 2-[(4aS,5aR)-5,5-difluoro-5a-methyl-1-(oxan-2-yl)-1H,4H,4aH,5H,5aH,6H-cyclopropa[f]indazol-3-yl]-5-fluoro-1H-indole-6-carboxylate (X₄₄a) (0.163 g, 31%) as a white solid. LCMS: C₂₄H₂₄F₃N₃O₃, desired mass=459.2 observed mass=460.1 [M+H]⁺.

### Step 3: Synthesis of 2-[(4aS,5aR)-5,5-difluoro-5a-methyl-1-(oxan-2-yl)-1H,4H,4aH,5H,5aH,6H-cyclopropa[f]indazol-3-yl]-5-fluoro-1H-indole-6-carboxylic acid (BBX₄₄)

To a solution of methyl 2-[(4aS,5aR)-5,5-difluoro-5a-methyl-1-(oxan-2-yl)-1H,4H,4aH,5H,5aH,6H-cyclopropa[f]indazol-3-yl]-5-fluoro-1H-indole-6-carboxylate (X₄₄a) (133 mg, 0.28 mmol) in a mixture of methanol/THF/water (1:1:1, v/v/v; 3 mL). was added LiOH*H₂O (176 mg, 4.2 mmol). The reaction mixture was stirred at 50° C. for 16 h. Additional 5 eq of LiOH·H₂O (58 mg) was added and the reaction mixture was stirred at 50° C. for another 5 h. After completion of the reaction, the solvent was evaporated, and the residue was partitioned between EtOAc (15 mL) and 1N HCl (10 mL). The layers were separated and the aqueous layer back-extracted with EtOAc (2×15 mL). The combined organic layer was washed with brine (10 mL). The organic layer was collected, dried over Na₂SO₄, filtered, and concentrated in vacuo. It was then triturated with DEE (2×5 mL) to yield 107 mg of the crude, which was purified by prep-HPLC to obtain 2-[(4aS,5aR)-5,5-difluoro-5a-methyl-1-(oxan-2-yl)-1H,4H,4aH,5H,5aH,6H-cyclopropa[f]indazol-3-yl]-5-fluoro-1H-indole-6-carboxylic acid (BBX₄₄) (44 mg, 42%) as a white solid. LCMS: C₂₃H₂₂F₃N₃O₃, desired mass=445.2 observed mass=444.3 [M−H]⁻. H NMR (300 MHz, DMSO-dS) δ 11.58 (d, J=5.1 Hz, 1H), 7.95 (d, J=6.3 Hz, 1H), 7.30 (d, J=12.0 Hz, 1H), 6.67 (d, J=2.3 Hz, 1H), 5.42 (ddd, J=18.1, 9.9, 2.5 Hz, 1H), 3.91 (d, J=9.2 Hz, 1H), 3.68 (d, J=13.4 Hz, 2H), 3.13-2.93 (m, 4H), 2.81 (d, J=17.3 Hz, 1H), 2.06 (d, J=18.7 Hz, 1H), 1.90 (t, J=11.1 Hz, 2H), 1.70 (s, 1H), 1.56 (s, 2H), 1.40 (s, 3H).

### Example 134. (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅)

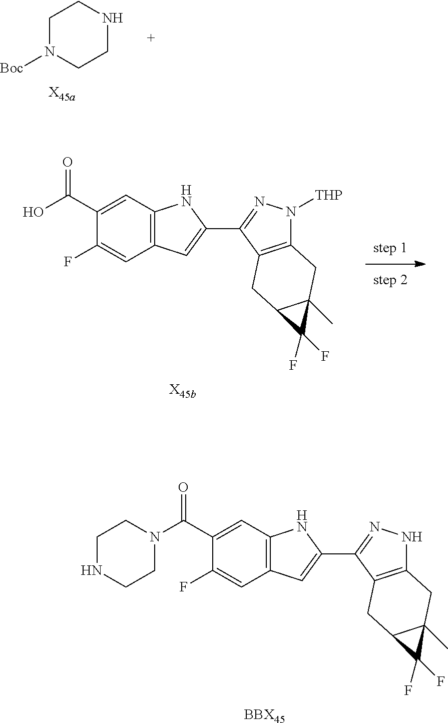

### Step 1: Synthesis of tert-butyl 4-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-5-fluoro-1H-indole-6-carbonyl)piperazine-1-carboxylate

To a vial was added tert-butyl piperazine-1-carboxylate (X₄₅ₐ) (16 mg, 0.084 mmol), 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-5-fluoro-1H-indole-6-carboxylic acid (X₄₅b) (35 mg, 0.079 mmol), EDCI (17 mg, 0.086 mmol) and HOBt (13 mg, 0.086 mmol) in DMF (0.4 mL). The reaction mixture was stirred for 16 hr, diluted with H₂O, extracted with EtOAc, dried over Na₂SO₄, and concentrated under reduced pressure. Flash chromatography (SiO₂, 0 to70% EtOAc/hexanes gradient elution) provided the title compound (51 mg, 0.083 mmol, quantative)

### Step 2: Synthesis of (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅)

Product from step 1 and HCl (4 M in dioxane, 0.8 mL, 3.3 mmol) were stirred at for 1.5 hr. The reaction mixture was concentrated under a positive flow of N₂. Flash chromatography (C18, 0 to 75% MeCN in H₂O) provided (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (26 mg, 0.061 mmol, 74%). ¹H NMR (400 MHz, MeOD) δ 7.44-7.39 (m, 1H), 7.37-7.30 (m, 1H), 6.71 (s, 1H), 3.84-3.77 (m, 2H), 3.44-3.39 (m, 2H), 3.25-3.08 (m, 3H), 2.98-2.91 (m, 2H), 2.89-2.80 (m, 3H), 1.82-1.72 (m, 1H), 1.47-1.42 (m, 3H). LCMS: C₂₂H₂₂F₃N₅O requires: 429, found: m/z=430 [M+H]⁺.

### Example 135. 6,6-dimethyl-N-[1-(piperidin-4-yl)-1H-pyrazol-4-yl]-4,5,6,7-tetrahydro-1H-indazole-3-carboxamide (BBX₄₆)

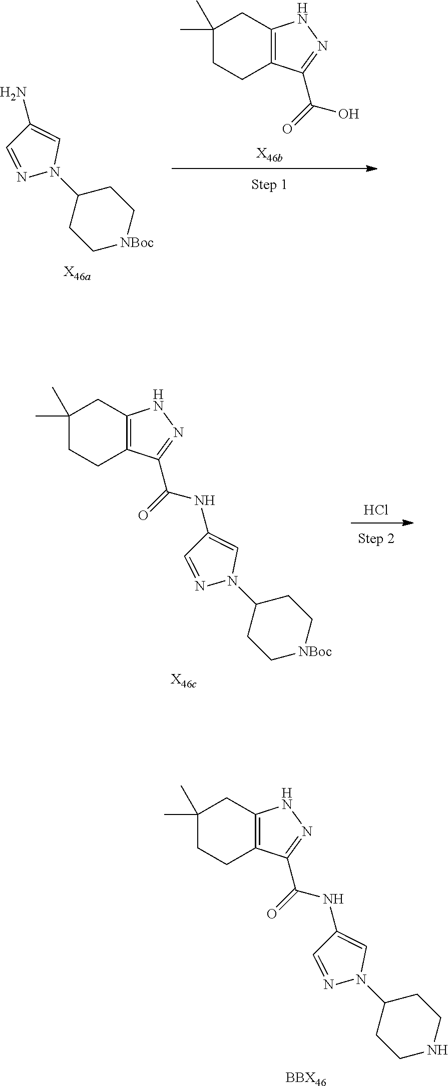

### Step 1: Synthesis of tert-butyl 4-[4-(6,6-dimethyl-4,5,6,7-tetrahydro-1H-indazole-3-amido)-1H-pyrazol-1-yl]piperidine-1-carboxylate (X_{46c})

To a mixture of 6,6-dimethyl-4,5,6,7-tetrahydro-1H-indazole-3-carboxylic acid (0.8 g, 3.82 mmol), trimethylamine (X_{46b}) (1.6 mL, 11.45 mmol) and the tert-butyl 4-(4-amino-1H-pyrazole-1-yl)piperidine-1-carboxylate (X₄₆a) (1.27 g, 9.09 mmol) in ethyl acetate (19 mL, 0.2 M) was slowly added T3P (50% in EtOAc, 3.65 ml, 5.73 mmol) at 0° C. The reaction mixture was slowly allowed to warm to room temperature and stirred for 10 h. The reaction mixture was chilled on an ice-water bath, and water (100 mL) was added dropwise and let stir for 25 min at room temperature, diluted with EtOAc (100 mL), and separated layers (aq pH-6). The organic layer was washed with sat NaHCO₃ (2×35 mL), brine (35 mL), dried over Na₂SO₄ and evaporated to give 4.1 g of crude as a pale brown foam. It was then purified by silica gel flash chromatography (MeOH/DCM, 0 to 5%) to give tert-butyl 4-[4-(6,6-dimethyl-4,5,6,7-tetrahydro-1H-indazole-3-amido)-1H-pyrazol-1-yl]piperidine-1-carboxylate (X_{46c}) (775 mg, 44%) as a white solid. LCMS: C₂₃H₃₄N₆O₃, desired mass=442.3 observed mass=441.1 [M−H]⁻. ¹H NMR (300 MHz, DMSO-d₆) δ 12.83 (s, 1H), 10.08 (s, 1H), 8.02 (s, 1H), 7.64 (s, 1H), 4.31 (td, J=11.4, 5.8 Hz, 1H), 4.10-3.94 (m, 2H), 3.17 (d, J=5.2 Hz, 1H), 2.89 (s, 2H), 2.67 (t, J=6.3 Hz, 2H), 2.39 (s, 2H), 1.97 (d, J=12.0 Hz, 2H), 1.74 (qd, J=12.2, 4.3 Hz, 2H), 1.48 (t, J=6.2 Hz, 2H), 1.42 (s, 9H), 0.97 (s, 6H).

### Step 2: Synthesis of 6,6-dimethyl-N-[1-(piperidin-4-yl)-1H-pyrazol-4-yl]-4,5,6,7-tetrahydro-1H-indazole-3-carboxamide (BBX₄₆)

To a solution of tert-butyl 4-[4-(6,6-dimethyl-4,5,6,7-tetrahydro-1H-indazole-3-amido)-1H-pyrazol-1-yl]piperidine-1-carboxylate (X_{46c}) (775 mg, 1.68 mmol, 1.0 eq) in a mixture of dichloromethane anhydrous (21 mL) and methanol (2.1 mL) was slowly added 4 M HCl in 1,4-dioxane (3.87 mL, 15.51 mmol, 7.0 eq). The reaction mixture was stirred at room temperature for 4 h. The solid was filtered off, washed 3 times with Et₂O on the filter and dried in vacuo overnight to yield 6,6-dimethyl-N-[1-(piperidin-4-yl)-1H-pyrazol-4-yl]-4,5,6,7-tetrahydro-1H-indazole-3-carboxamide (BBX₄₆) (618 mg, 96%, as hydrochloride) as white solid. LCMS: C₁₈H₂₆N₆O requires 342.2 observed mass=343.7 [M+H]⁺. ¹H NMR (300 MHz, DMSO-d₆) δ 10.17 (s, 1H), 9.32 (d, J=11.1 Hz, 1H), 9.07 (d, J=10.8 Hz, 1H), 8.03 (s, 1H), 7.68 (s, 1H), 4.44 (q, J=7.5 Hz, 1H), 3.41-3.27 (m, 2H), 3.03 (s, 2H), 2.67 (t, J=6.4 Hz, 2H), 2.39 (s, 2H), 2.14 (q, J=5.6, 5.1 Hz, 4H), 1.47 (t, J=6.4 Hz, 2H), 0.96 (s, 6H).

### Example 136. (1R,4R)-4-[4-(6,6-dimethyl-4,5,6,7-tetrahydro-1H-indazole-3-amido)-1H-pyrazol-1-yl]cyclohexane-1-carboxylic acid (BBX₄₇)

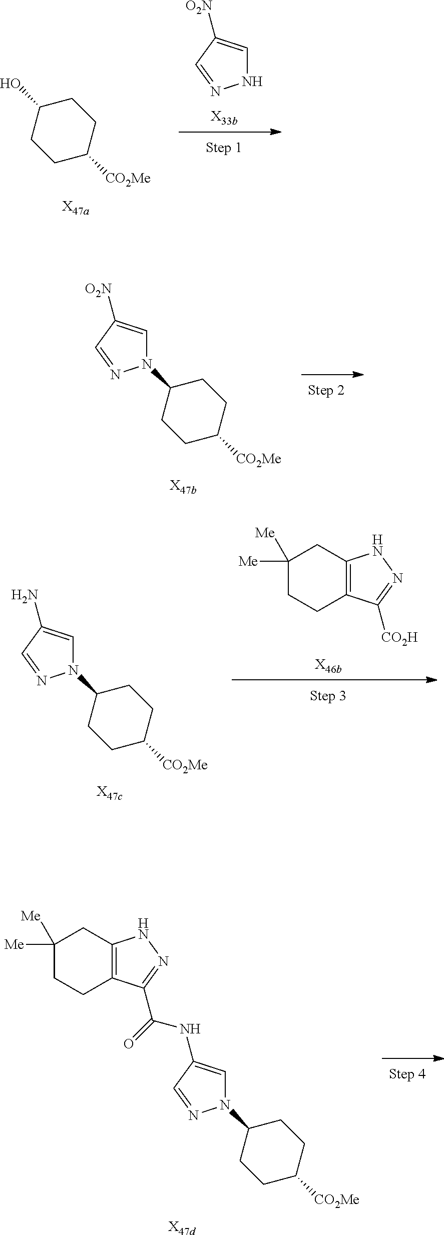

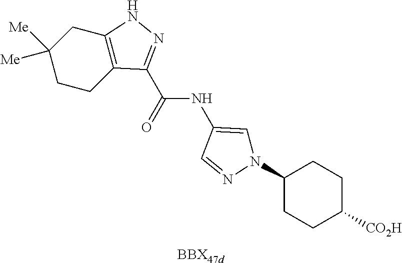 [SMILES: CC1(C)CCc2c(C(=O)Nc3cnn([C@H]4CC[C@H](C(=O)O)CC4)c3)n[nH]c2C1 ; "BBX47d"]

### Step 1: Synthesis of methyl (1R,4R)-4-(4-nitro-1H-pyrazol-1-yl)cyclohexane-1-carboxylate (X_{47b})

To a stirred solution of 4-nitro-1H-pyrazole (X_{33b}) (1.20 g, 10.612 mmol, 1.0 equiv.), methyl (1S,4S)-4-hydroxycyclohexane-1-carboxylate (X₄₇a) (2.02 g, 12.735 mmol, 1.2 equiv.), triphenylphosphine (5.57 g, 21.225 mmol, 2.0 equiv.) and triethylamine (1.8 mL, 12.735 mmol, 1.2 equiv.) in anhydrous tetrahydrofuran (53 mL, 0.2 M), was added diisopropyl azodicarboxylate (4.3 g, 21.225 mmol, 2.0 equiv.) slowly over three hours at 0° C. under argon atmosphere. The reaction mixture was allowed to warm up to room temperature and stirred overnight. Next, the solvent was removed under reduced pressure and the crude residue was diluted in DCM and precipitated by the addition of n-hexane to provide methyl (1R,4R)-4-(4-nitro-1H-pyrazol-1-yl)cyclohexane-1-carboxylate (X_{47b}) (0.824 g, 31%) as a pale-yellow solid. LCMS: m/z (ESI+) calculated for [C₁₁H₁₅N₃O₄+H+MeCN]⁺: 295.14, found: 295.10. ¹H NMR (300 MHz, DMSO-d₆): δ 8.90 (d, J=0.7 Hz, 1H), 8.27 (d, J=0.7 Hz, 1H), 4.28 (tt, J=12.0, 3.7 Hz, 1H), 3.61 (s, 3H), 2.39 (tt, J=11.9, 3.5 Hz, 1H), 2.06 (td, J=15.5, 3.6 Hz, 4H), 1.81 (qd, J=13.8, 13.0, 3.9 Hz, 2H), 1.60-1.44 (m, 2H).

### Step 2: Synthesis of methyl (1R,4R)-4-(4-amino-1H-pyrazol-1-yl)cyclohexane-1-carboxylate (X_{47c})

A solution of methyl (1R,4R)-4-(4-nitro-1H-pyrazol-1-yl)cyclohexane-1-carboxylate (X_{47b}) (0.824 g, 3.2407 mmol, 1.0 equiv.) in methanol (108 ml, 0.03 M) was degassed under argon. To this, palladium hydroxide on carbon (0.100 g, 0.7130 mmol, 0.22 equiv.) was added. The reaction mixture was purged 3 times with vacuum/hydrogen cycles and left stirring under hydrogen (1 atm) over weekend at room temperature. The mixture was filtered through a celite pad, washed with MeOH (3×50 mL), the filtrate was evaporated to dryness to give crude Methyl (1R,4R)-4-(4-amino-1H-pyrazol-1-yl)cyclohexane-1-carboxylate (X_{47c}) (709.4 mg, 98% yield) that was used for the next step without further purification. LCMS: m/z (ESI+) calculated for [C₁₁H₁₇N₃O₂+H]⁺: 224.14, found: 224.24. ¹H NMR (300 MHz, DMSO-d₆): δ 7.03 (d, J=0.9 Hz, 1H), 6.89 (d, J=0.9 Hz, 1H), 4.06 (br-s, 2H), 3.94 (tt, J=11.7, 3.6 Hz, 1H), 3.61 (s, 3H), 2.37 (tt, J=11.9, 3.3 Hz, 1H), 2.05-1.92 (m, 4H), 1.76-1.60 (m, 2H), 1.57-1.41 (m, 2H).

### Step 3: Synthesis of methyl (1R,4R)-4-[4-(6,6-dimethyl-4,5,6,7-tetrahydro-1H-indazole-3-amido)-1H-pyrazol-1-yl]cyclohexane-1-carboxylate (X₄₇a)

To a solution of 6,6-dimethyl-4,5,6,7-tetrahydro-1H-indazole-3-carboxylic acid (X_{46b}) (1.36 g, 6.49 mmol, 1.0 equiv.), triethylamine (2.7 ml, 19.47 mmol, 3.0 equiv.), and methyl (1R,4R)-4-(4-amino-1H-pyrazol-1-yl)cyclohexane-1-carboxylate (X_{47c}) (1.65 g, 6.49 mmol, 1.0 equiv.) in anhydrous dimethylformamide (32.5 mL, 0.2 M) was slowly added propanephosphonic acid anhydride (50% solution in EtOAc, 12.4 mL, 20.83 mmol, 3.21 equiv.) at 0° C. The reaction mixture was slowly allowed to warm to room temperature and stirred for 16 h. Then, the reaction mixture was chilled on an ice-water bath, water (50 mL) was added dropwise and left stirring for 25 min at room temperature, diluted with EtOAc (50 mL) and layers were separated (aq pH-6). The organic layer was washed with saturated NaHCO₃ (3×50 mL), brine (50 mL), dried over Na₂SO₄, and concentrated under reduced pressure. The crude was purified by silica gel chromatography (0 to 2.5% OMeOH in DCM) and subsequent precipitation using DCM and hexanes to provide methyl (1R,4R)-4-[4-(6,6-dimethyl-4,5,6,7-tetrahydro-1H-indazole-3-amido)-1H-pyrazol-1-yl]cyclohexane-1-carboxylate (X₄₇a) 9920 mg, 35%) as a pale-pink solid. LCMS: m/z (ESI+) calculated for [C₂₁H₂₉N₅O₃+H]⁺: 400.23, found: 400.55. ¹H NMR (300 MHz, methanol-d₄): δ 8.04 (s-ap, 1H), 7.67 (s-ap, 1H), 4.14 (tt, J=12.1, 3.9 Hz, 1H), 3.68 (s, 3H), 2.79 (t, J=6.4 Hz, 2H), 2.49-2.44 (m, 2H), 2.16 (m, 5H), 1.92-1.76 (m, 2H), 1.73-1.60 (m, 2H), 1.57 (t, J=6.4 Hz, 2H), 1.03 (s, 6H).

### Step 4: Synthesis of (1R,4R)-4-[4-(6,6-dimethyl-4,5,6,7-tetrahydro-1H-indazole-3-amido)-1H-pyrazol-1-yl]cyclohexane-1-carboxylic acid (BBX₄₇)

To a solution of methyl (1R,4R)-4-[4-(6,6-dimethyl-4,5,6,7-tetrahydro-1H-indazole-3-amido)-1H-pyrazol-1-yl]cyclohexane-1-carboxylate (X₄₇a) (0.36 g, 0.9 mmol, 1.0 equiv.) in a mixture of methanol: tetrahydrofuran: water (1:1:1 v/v/v; 13.5 mL, 0.2 M) was added lithium hydroxide monohydrate (0.756 g, 18.02 mmol, 20.0 equiv.). The reaction mixture was stirred at 50° C. for 16 h. After completion of the reaction, the solvent was evaporated and HCl 1N was added to the aqueous phase until pH 4-6. The compound was extracted using EtOAc (3×30 mL) and the organic phase was washed with brine, dried over Na₂SO₄, filtered, and concentrated in vacuo. The crude was purified by trituration using water, diethyl ether, and pentane to provide (1R,4R)-4-[4-(6,6-dimethyl-4,5,6,7-tetrahydro-1H-indazole-3-amido)-1H-pyrazol-1-yl]cyclohexane-1-carboxylic acid (BBX₄₇) (224 mg, 63% yield) as a pale-pink solid. LCMS: Method: LCMS-019-10-70-95-6-1-25-UV; RT 2.99 min; purity: 98.67% at 254 nm; m/z (ESI+) calculated for [C₂₀H₂₇N₅O₃+H]⁺: 386.22, found: 385.93. ¹H NMR (300 MHz, methanol-d₄): δ 8.02 (s, 1H), 7.65 (s, 1H), 4.21-4.09 (m, 1H), 2.79 (t, J=6.4 Hz, 2H), 2.46-2.33 (m, 3H), 2.24-2.10 (m, 4H), 1.85 (qd, J=12.8, 3.4 Hz, 2H), 1.71-1.61 (m, 2H), 1.57 (t, J=6.4 Hz, 2H), 1.03 (s, 6H).

### Example 137. 6,6-dimethyl-N-{1-[(1R,4R)-4-formylcyclohexyl]-1H-pyrazol-4-yl}-4,5,6,7-tetrahydro-1H-indazole-3-carboxamide (BBX₄₈)

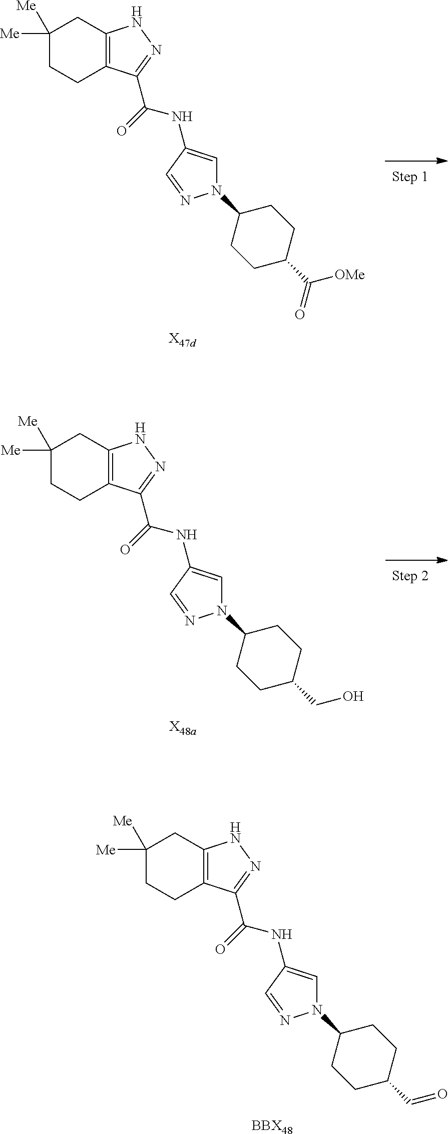

### Step 1: Synthesis of 6,6-dimethyl-N-{1-[(1R,4R)-4-(hydroxymethyl)cyclohexyl]-1H-pyrazol-4-yl}-4,5,6,7-tetrahydro-1H-indazole-3-carboxamide (X₄₈ₐ)

To a solution of methyl (1R,4R)-4-[4-(6,6-dimethyl-4,5,6,7-tetrahydro-1H-indazole-3-amido)-1H-pyrazol-1-yl]cyclohexane-1-carboxylate (X₄₇a) (0.64 g, 1.6 mmol, 1.0 equiv.) in a mixture of anhydrous tetrahydrofuran (16 mL, 0.1 M) and methanol (3.2 mL, 0.5 M) was added lithium borohydride (0.098 g, 4.5 mmol, 3.21 equiv.) at 0° C. Then, the mixture was stirred at 50° C. for 3 hours. After completion of the reaction, it was quenched by the addition of water (30 mL), extracted with DCM (3×30 mL). The organic layers were combined, washed with brine (2×40 mL), dried with anhydrous Na₂SO₄, filtered, and concentrated in vacuo. The resulting crude was purified by silica gel chromatography (0 to 5% MeOH in DCM) to give 6,6-dimethyl-N-{1-[(1R,4R)-4-(hydroxymethyl)cyclohexyl]-1H-pyrazol-4-yl}-4,5,6,7-tetrahydro-1H-indazole-3-carboxamide (X₄₈ₐ) (440 mg, 74%) as a white solid. LCMS: m/z (ESI+) calculated for [C₂₀H₂₉N₅O₂+H]⁺: 372.24, found: 372.55. ¹H NMR (300 MHz, Methanol-d₄): δ 8.02 (d, J=0.7 Hz, 1H), 7.64 (d, J=0.7 Hz, 1H), 4.11 (tt, J=12.0, 3.9 Hz, 1H), 3.43 (d, J=6.3 Hz, 2H), 2.79 (t, J=6.4 Hz, 2H), 2.44 (s, 2H), 2.16 (d, J=11.9 Hz, 2H), 1.98 (d, J=13.3 Hz, 2H), 1.81 (m, 2H), 1.57 (t, J=6.4 Hz, 3H), 1.19 (m, 2H), 1.03 (s, 6H).

### Step 2: Synthesis of 6,6-dimethyl-N-{1-[(1R,4R)-4-formylcyclohexyl]-1H-pyrazol-4-yl}-4,5,6,7-tetrahydro-1H-indazole-3-carboxamide (BBX₄₈)

To a solution of 6,6-dimethyl-N-{1-[(1R,4R)-4-(hydroxymethyl) cyclohexyl]-1H-pyrazol-4-yl}-4,5,6,7-tetrahydro-1H-indazole-3-carboxamide (X₄₈ₐ) (0.44 g, 1.18 mmol, 1.0 equiv.) in dichloromethane anhydrous (11.8 mL, 0.1 M) at 0° C. was added Dess-Martin periodinane (0.41 g, 0.973 mmol, 1.3 equiv.). Then, the mixture was stirred at room temperature for 30 min under argon. On the completion, the reaction was concentrated under reduced pressure. The resulting crude was purified by prep-HPLC using C18 column and ACN+0.1% FA, H₂O+0.1% FA as mobile phase to provide 6,6-dimethyl-N-{1-[(1R,4R)-4-formylcyclohexyl]-1H-pyrazol-4-yl}-4,5,6,7-tetrahydro-1H-indazole-3-carboxamide (BBX₄₈) (41.9 mg, 10%) as white solid. LCMS: Method: LCMS2-043-20-50-95-10-05-55-UV; RT=5.78 min; purity at 254 nm: 95.07%; m/z (ESI+) calculated for [C₂₀H₂₈N₅O₂+H]⁺: 370.2, found: 370.2. ¹H NMR (300 MHz, Methanol-d₄): δ 8.01 (s, 1H), 7.64 (d, J=0.7 Hz, 1H), 4.26 (d, J=5.8 Hz, 1H), 4.16-4.00 (m, 2H), 2.79 (t, J=6.3 Hz, 2H), 2.44 (s, 2H), 2.15 (d, J=7.3 Hz, 2H), 2.03 (d, J=12.7 Hz, 2H), 1.85-1.69 (m, 3H), 1.57 (t, J=6.4 Hz, 3H), 1.31 (d, J=8.6 Hz, 2H), 1.03 (s, 6H).

### Example 138. (1R,4R)-4-{4-[(4aS,5aR)-5,5-difluoro-5a-methyl-1H,4H,4aH,5H,5aH,6H-cyclopropa[f] indazole-3-amido]-1H-pyrazol-1-yl}cyclohexane-1-carboxylic acid (BBX₄₉)

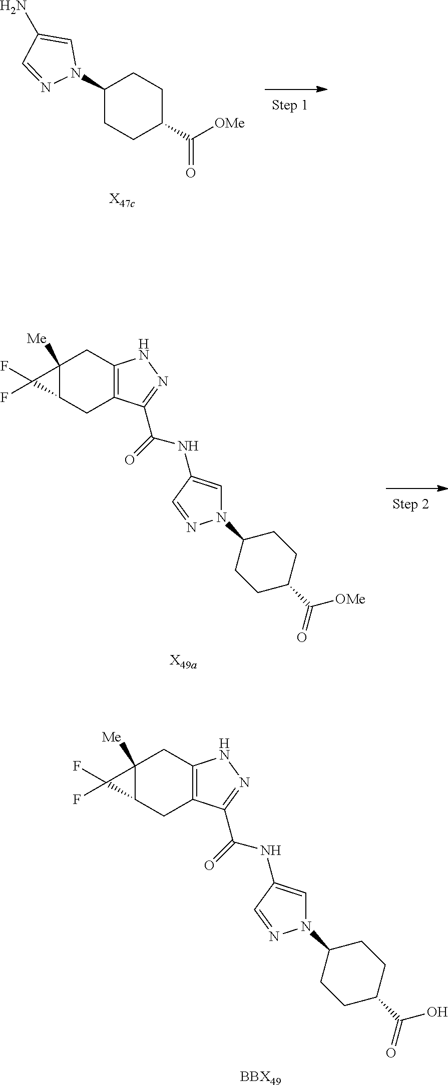

### Step 1: Synthesis of methyl (1R,4R)-4-{4-[(4aS,5aR)-5,5-difluoro-5a-methyl-1H,4H,4aH,5H,5aH,6H-cyclopropa[f]indazole-3-amido]-1H-pyrazol-1-yl}cyclohexane-1-carboxylate (X₄₉ₐ)

To a mixture of the (4aS,5aR)-5,5-difluoro-5a-methyl-1H,4H,4aH,5H,5aH,6H-cyclopropa[f]indazole-3-carboxylic acid (X₃₃ₑ) (0.92 g, 3.87 mmol, 1.0 equiv.), triethylamine (1.6 mL, 11.5 mmol, 3.0 equiv.) and methyl (1R,4R)-4-(4-amino-1H-pyrazol-1-yl)cyclohexane-1-carboxylate (X_{47c}) (1.2 g, 4.72 mmol, 1.2 equiv.) in ethyl acetate (19.4 mL, 0.2 M) was slowly added propanephosphonic acid anhydride (4.9 mL, 7.6 mmol, 2.0 equiv.) at 0° C. The reaction mixture was slowly allowed to warm to room temperature and stirred for 16 h. On completion, the reaction mixture was chilled on an ice-water bath, water (40 mL) was added dropwise, and let stirring for 10 min at room temperature. Then, the aqueous phase was extracted with ethyl acetate (3×40 mL) and layers were separated (aq pH-6). The organic layer was washed with a saturated aqueous solution of NaHCO₃ (30 mL), brine (30 mL), dried over Na₂SO₄, and concentrated in vacuo. The crude was diluted in ethyl acetate and precipitated by the addition of n-hexane to provide methyl (1R,4R)-4-{4-[(4aS,5aR)-5,5-difluoro-5a-methyl-1H,4H,4aH,5H,5aH,6H-cyclopropa[f]indazole-3-amido]-1H-pyrazol-1-yl}cyclohexane-1-carboxylate (X₄₉ₐ) (1.28 g, 76%) as a pale-yellow solid. LCMS: m/z (ESI+) calculated for [C₂₁H₂₅N₅O₃+H]⁺: 434.2, found: 434.2. ¹H NMR (300 MHz, DMSO-d₆): δ 12.96 (s, 1H), 10.14 (s, 1H), 8.00 (s, 1H), 7.60 (s, 1H), 4.17-4.04 (m, 1H), 3.61 (s, 3H), 3.03 (d, J=18.6 Hz, 3H), 2.87-2.76 (m, 1H), 2.44-2.34 (m, 1H), 2.02 (d, J=10.6 Hz, 4H), 1.84-1.64 (m, 3H), 1.61-1.43 (m, 2H), 1.35 (s, 3H).

### Step 2: Synthesis of (1R,4R)-4-{4-[(4aS,5aR)-5,5-difluoro-5a-methyl-1H,4H,4aH,5H,5aH,6H-cyclopropa[f] indazole-3-amido]-1H-pyrazol-1-yl}cyclohexane-1-carboxylic acid (BBX₄₉)

To a mixture of methyl (1R,4R)-4-{4-[(4aS,5aR)-5,5-difluoro-5a-methyl-1H,4H,4aH,5H,5aH,6H-cyclopropa[f]indazole-3-amido]-1H-pyrazol-1-yl}cyclohexane-1-carboxylate (X₄₉ₐ) (0.3 g, 0.692 mmol, 1 equiv.) in mixture methanol/tetrahydrofuran/water (1:1:1 v:v:v; 10.4 mL, 0.07 M) at 0° C. was added lithium hydroxide monohydrate (0.58 g, 13.84 mmol, 20.0 equiv.) and the reaction mixture was stirred at 50° C. for 16 h. After completion of the reaction, the volatiles was evaporated, and the residue was partitioned between EtOAc and 1N aq. HCl. The organic layer was separated and concentrated in vacuo. The residue was taken into water (30 mL) and extracted with ethyl acetate (3×30 mL). The organic layers were washed with brine, dried over Na₂SO₄, and concentrated in vacuo. The crude was triturated using water, diethyl ether, and pentane and to provide (1R,4R)-4-{4-[(4aS,5aR)-5,5-difluoro-5a-methyl-1H,4H,4aH,5H,5aH,6H-cyclopropa[f] indazole-3-amido]-1H-pyrazol-1-yl}cyclohexane-1-carboxylic acid (BBX₄₉) (181 mg, 62%) as a tan solid. LCMS: m/z (ESI+) calculated for [C₂₀H₂₃F₂N₅O₂+H]⁺: 420.1, found: 420.2. ¹H NMR (300 MHz, Methanol-d₄) δ 8.02 (s, 1H), 7.65 (s, 1H), 4.20-4.09 (m, 1H), 3.18-3.03 (m, 3H), 2.80 (dd, J=17.0, 3.5 Hz, 1H), 2.44-2.33 (m, 1H), 2.22-2.12 (m, 4H), 1.92-1.77 (m, 2H), 1.71-1.56 (m, 3H), 1.39 (d, J=2.2 Hz, 3H).

### Example 139. 2-(6,6-Dimethyl-1,4,5,7-tetrahydroindazol-3-yl)-1H-indole-5-carboxylic acid

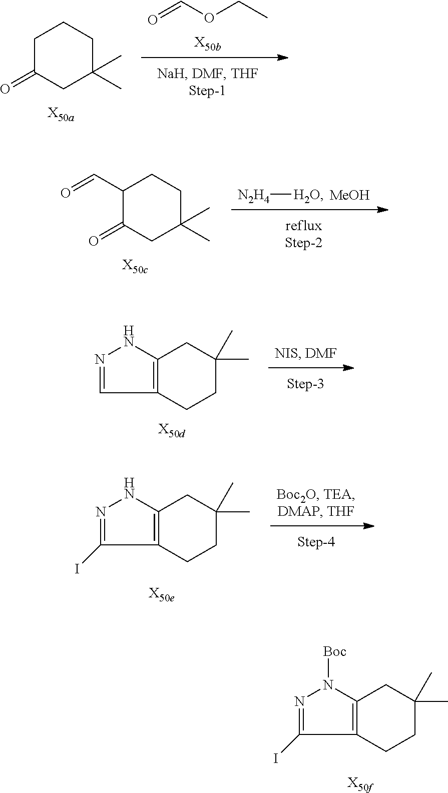

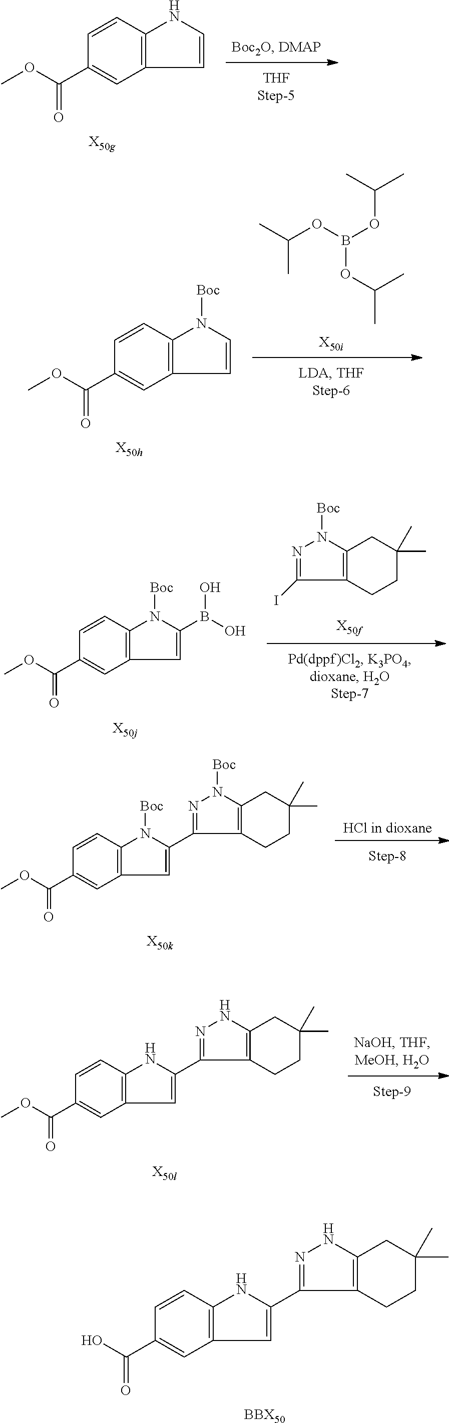

### Step 1: Synthesis of 4,4-dimethyl-2-oxocyclohexane-1-carbaldehyde (X_{50c})

To a mixture of cyclohexanone, 3,3-dimethyl (X₅₀ₐ) (22 g, 174.328 mmol, 1 equiv) in THF (100 mL) was added NaH (4.6 g, 191.761 mmol, 1.1 equiv) for 1 h at 0° C. under nitrogen atmosphere followed by the addition of ethyl formate (X_{50b}) (25.83 g, 348.656 mmol, 2 equiv) dropwise at 0° C. The resulting mixture was stirred at room temperature for 1 h under nitrogen atmosphere. To the above mixture was added DMF (20 mL) at room temperature. The resulting mixture was stirred at room temperature overnight. The reaction was quenched by the addition of saturated NH₄Cl aqueous solution. The resulting mixture was extracted with EtOAc. The combined organic layers were washed with brine, dried over anhydrous Na₂SO₄. After filtration, the filtrate was concentrated under reduced pressure. This resulted in 4,4-dimethyl-2-oxocyclohexane-1-carbaldehyde (X_{50c}) as a yellow oil (27 g, crude). MS (ESI) calc'd for (C₉H₁₄O₂) [M+1]⁺, 155.1; found, 155.0.

### Step 2: Synthesis of 6,6-dimethyl-1,4,5,7-tetrahydroindazole (X₅₀ₐ)

To a mixture of 4,4-dimethyl-2-oxocyclohexane-1-carbaldehyde (X_{50c}) (21 g, 136.179 mmol, 1 equiv) in MeOH (100 mL) was added hydrazine hydrate (98%) (8.18 g, 163.415 mmol, 1.2 equiv). The resulting mixture was stirred at 80° C. for 3 h. The resulting mixture was concentrated under vacuum. The residue was dissolved in water (200 mL). The resulting mixture was extracted with EtOAc. The combined organic layers were washed with brine, dried over anhydrous Na₂SO₄. After filtration, the filtrate was concentrated under reduced pressure. This resulted in 6,6-dimethyl-1,4,5,7-tetrahydroindazole (X₅₀ₐ) as a light yellow solid (16 g, crude). MS (ESI) calc'd for (C₉H₁₄N₂) [M+1]⁺, 151.1; found, 151.2.

### Step 3: Synthesis of 3-iodo-6,6-dimethyl-1,4,5,7-tetrahydroindazole (X_{50c})

To a mixture of 6,6-dimethyl-1,4,5,7-tetrahydroindazole (X₅₀ₐ) (16 g, 106.507 mmol, 1 equiv) in DMF (100 mL) was added NIS (35.94 g, 159.761 mmol, 1.5 equiv) at 0° C. The resulting mixture was stirred at 80° C. for 2 h. The mixture was allowed to cool down to room temperature. The resulting mixture was added saturated Na₂S₂O₃ aqueous solution. The resulting mixture was extracted with CH₂Cl₂. The combined organic layers were washed with brine, dried over anhydrous Na₂SO₄. After filtration, the filtrate was concentrated under reduced pressure. This resulted in 3-iodo-6,6-dimethyl-1,4,5,7-tetrahydroindazole ((X_{50c}) as a yellow solid (14 g, 38.08%). MS (ESI) calc'd for (C₉H₁₃IN₂) [M+1]⁺, 277.1; found, 277.2.

### Step 4: Synthesis of tert-butyl 3-iodo-6,6-dimethyl-5,7-dihydro-4H-indazole-1-carboxylate (X₅₀f)

To a mixture of 3-iodo-6,6-dimethyl-1,4,5,7-tetrahydroindazole (X_{50c}) (32 g, 115.891 mmol, 1 equiv) and Boc₂O (25.29 g, 115.891 mmol, 1 equiv) in THE (150 mL) were added TEA (11.73 g, 115.891 mmol, 1 equiv) and DMAP (0.57 g, 4.636 mmol, 0.04 equiv) at 0° C. The resulting mixture was stirred at room temperature for 2 h. The resulting mixture was concentrated under vacuum. The residue was purified by silica gel column chromatography, eluted with PE/EA (1/1). This resulted in tert-butyl 3-iodo-6,6-dimethyl-5,7-dihydro-4H-indazole-1-carboxylate (X₅₀f) as a yellow solid (36 g, 74.31%). MS (ESI) calc'd for (C₁₄H₂₁ IN₂02) [M+1]⁺, 377.1; found, 377.2

### Step 5: Synthesis of tert-butyl-5-methyl indole-1,5-dicarboxylate (X₅₀ₕ)

To a mixture of methyl 1H-indole-5-carboxylate (X_{50g}) (25 g, 142.705 mmol, 1 equiv) and Boc₂O (34.26 g, 156.976 mmol, 1.1 equiv) in THF (20 mL) was added DMAP (1.74 g, 14.271 mmol, 0.1 equiv) at 0° C. The resulting mixture was stirred at room temperature for 1 h. The resulting mixture was concentrated under vacuum. The residue was purified by silica gel column chromatography, eluted with PE/EA (1/1). This resulted in 1-tert-butyl 5-methyl indole-1,5-dicarboxylate (X₅₀ₕ) as a colorless oil (37.2 g, 85.22%). MS (ESI) calc'd for (C₁₅H₁₇NO₄) [M+1]⁺, 276.1; found, 276.1.

Step 6: Synthesis of 1-(tert-butoxycarbonyl)-5-(methoxycarbonyl)indol-2-ylboronic acid (X₅₀ⱼ)

To a mixture of 1-tert-butyl 5-methyl indole-1,5-dicarboxylate (X₅₀ₕ) (20 g, 72.647 mmol, 1 equiv) and triisopropyl borate (X₅₀ᵢ) (20.49 g, 108.971 mmol, 1.5 equiv) in THE (100 mL) was added LDA (47.2 mL, 94.441 mmol, 1.3 equiv, 2M) dropwise at 0° C. under nitrogen atmosphere. The resulting mixture was stirred at 0° C. for 2 h under nitrogen atmosphere. The mixture was neutralized to pH 7 with HCl (2 N) aqueous. The organic layer was separated and dried over anhydrous Na₂SO₄. After filtration, the filtrate was concentrated under reduced pressure. This resulted in 1-(tert-butoxycarbonyl)-5-(methoxycarbonyl)indol-2-ylboronic acid (X₅₀ⱼ) as a yellow solid (20 g, crude). MS (ESI) calc'd for (C₁₅H₁₈BNO₆) [M+1]⁺, 320.1; found, 320.1.

### Step 7: Synthesis of 1-tert-butyl 5-methyl 2-[1-(tert-butoxycarbonyl)-6,6-dimethyl-5,7-dihydro-4H-indazol-3-yl]indole-1,5-dicarboxylate (X₅₀ₙ)

To a mixture of 1-(tert-butoxycarbonyl)-5-(methoxycarbonyl)indol-2-ylboronic acid (X₅₀ⱼ) (20 g, 62.672 mmol, 1 equiv) and tert-butyl 3-iodo-6,6-dimethyl-5,7-dihydro-4H-indazole-1-carboxylate (X₅₀f) (23.58 g, 62.672 mmol, 1 equiv) in dioxane (200 mL) were added K₃PO₄ (53.21 g, 250.688 mmol, 4 equiv) in H₂O (20 mL) and Pd(dppf)C₁₂ (4.59 g, 6.267 mmol, 0.1 equiv). The resulting mixture was stirred at 110° C. for 4 h under nitrogen atmosphere. The resulting mixture was concentrated under vacuum. The residue was purified by silica gel column chromatography, eluted with PE/EA (1/1). This resulted in 1-tert-butyl 5-methyl 2-[1-(tert-butoxycarbonyl)-6,6-dimethyl-5,7-dihydro-4H-indazol-3-yl]indole-1,5-dicarboxylate (X_{50ck}) as a brown solid (10 g, 25.90%). MS (ESI) calc'd for (C₂₉H₃₇N₃O₆) [M+1]⁺, 524.3; found, 524.2.

### Step 8: Synthesis of methyl 2-(6,6-dimethyl-1,4,5,7-tetrahydroindazol-3-yl)-1H-indole-5-carboxylate (X₅₀ᵢ)

A mixture of 1-tert-butyl 5-methyl 2-[1-(tert-butoxycarbonyl)-6,6-dimethyl-5,7-dihydro-4H-indazol-3-yl]indole-1,5-dicarboxylate (X₅₀ₙ) (20 g, 38.195 mmol, 1 equiv) in HCl (gas)/1,4-dioxane (400 mL, 4N) was stirred at room temperature overnight. The resulting mixture was concentrated under vacuum. This resulted in methyl 2-(6,6-dimethyl-1,4,5,7-tetrahydroindazol-3-yl)-1H-indole-5-carboxylate (X₅₀ᵢ) as a yellow solid (16.5 g, crude). MS (ESI) calc'd for (C₁₉H₂₁N₃O₂) [M+1]⁺, 324.2; found, 324.1.

### Step 9: Synthesis of 2-(6,6-dimethyl-1,4,5,7-tetrahydroindazol-3-yl)-1H-indole-5-carboxylic acid (BBX₅₀)

To a mixture of methyl 2-(6,6-dimethyl-1,4,5,7-tetrahydroindazol-3-yl)-1H-indole-5-carboxylate (X₅₀ᵢ) (16.5 g, 51.021 mmol, 1 equiv) in THE (100 mL), MeOH (100 mL) and H₂O (100 mL) was added NaOH (20.41 g, 510.210 mmol, 10 equiv) at 0° C. The resulting mixture was stirred at 65° C. overnight. The resulting mixture was concentrated under vacuum. The residue was dissolved in water. The mixture was acidified to pH 7 with HCl (2N) aqueous. The precipitated solids were collected by filtration and washed with water. This resulted in 2-(6,6-dimethyl-1,4,5,7-tetrahydroindazol-3-yl)-1H-indole-5-carboxylic acid (BBX₅₀) as a yellow solid (11 g, 68.85%). MS (ESI) calc'd for (C₁₈H₁₉N₃O₂) [M+1]⁺, 310.2; found, 310.1. ¹H NMR (400 MHz, DMSO-d₆) δ 11.74 (s, 1H), 8.19 (s, 1H), 7.74-7.67 (m, 1H), 7.41 (d, J=8.4 Hz, 1H), 6.69 (s, 1H), 2.73-2.65 (m, 2H), 2.42 (s, 2H), 1.63-1.55 (m, 2H), 1.01 (s, 6H).

### Example 140. 2-[(4aS,5aR)-5,5-Difluoro-5a-methyl-1H,4H,4aH,6H-cyclopropa[f]indazol-3-yl]-1H-indole-5-carboxylic acid (BBX₅₁)

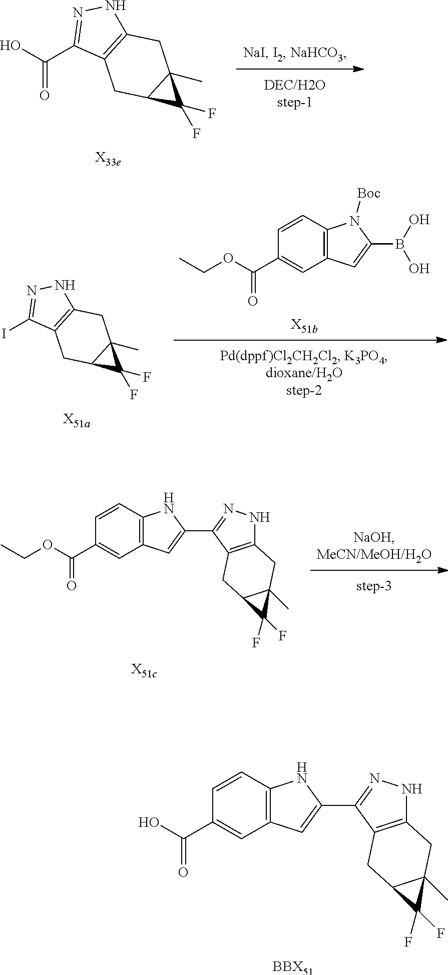

### Step 1: Synthesis of (4aS,5aR)-5,5-Difluoro-3-iodo-5a-methyl-1H,4H,4aH,6H-cyclopropa[f]indazole (X₅₁ₐ)

To a mixture of (4aS,5aR)-5,5-difluoro-5a-methyl-1H,4H,4aH,6H-cyclopropa[f]indazole-3-carboxylic acid (X₃₃ₑ) (1 g, 4.382 mmol, 1 equiv) and NaHCO₃ (1.2 g, 14.461 mmol, 3.3 equiv) in DCE (6 mL), H₂O (6 mL) were added NaI (1.6 g, 10.955 mmol, 2.5 equiv) and 1₂ (1.4 g, 5.697 mmol, 1.3 equiv) at room temperature. The resulting mixture was stirred for 4 h at 100° C. under nitrogen atmosphere. The resulting mixture was extracted with CH₂Cl₂. The combined organic layers were washed with aqueous Na₂S₂O₃, dried over anhydrous Na₂SO₄. After filtration, the filtrate was concentrated under reduced pressure. The residue was purified by silica gel column chromatography, eluted with CH₂Cl₂/MeOH (10/1) to afford (4aS,5aR)-5,5-difluoro-3-iodo-5a-methyl-1H,4H,4aH,6H-cyclopropa[f]indazole (X₅₁ₐ) (1.1 g, 80.95%) as a white solid. MS (ESI) calc'd for (C₉H₉F₂IN₂) [M+1]⁺, 311.0; found, 310.9.

### Step 2: Synthesis of 1-tert-Butyl 5-ethyl 2-[(4aS,5aR)-5,5-difluoro-5a-methyl-1H,4H,4aH,6H-cyclopropa[f]indazol-3-yl]indole-1,5-dicarboxylate (X_{51c})

To a mixture of (4aS,5aR)-5,5-difluoro-3-iodo-5a-methyl-1H,4H,4aH,6H-cyclopropa[f]indazole (X_{51b}) (1.8 g, 5.805 mmol, 1 equiv) in dioxane (21 mL) and H₂O (9 mL) were added 1-(tert-butoxycarbonyl)-5-(ethoxycarbonyl)indol-2-ylboronic acid (X_{51b}) (3.8 g, 11.610 mmol, 2 equiv), Pd(dppf)C₁₂CH₂Cl₂ (0.5 g, 0.581 mmol, 0.1 equiv) and K₃PO₄ (4.9 g, 23.220 mmol, 4 equiv) at room temperature. The resulting mixture was stirred for 30 min at 110° C. under nitrogen atmosphere. The resulting mixture was concentrated under reduced pressure. The residue was purified by silica gel column chromatography, eluted with CH₂Cl₂/EA (1/1) to afford 1-tert-butyl 5-ethyl 2-[(4aS,5aR)-5,5-difluoro-5a-methyl-1H,4H,4aH,6H-cyclopropa[f]indazol-3-yl]indole-1,5-dicarboxylate (X_{51c}) (650 mg, 23.75%) as a yellow solid. MS (ESI) calc'd for (C₂₀H₁₉F₂N₃O₂) [M+1]⁺, 372.1; found, 372.2.

### Step-3: Synthesis of 2-[(4aS,5aR)-5,5-Difluoro-5a-methyl-1H,4H,4aH,6H-cyclopropa[f]indazol-3-yl]-1H-indole-5-carboxylic acid (BBX₅₁)

To a mixture of methyl 2-[(4aS,5aR)-5,5-difluoro-5a-methyl-1H,4H,4aH,6H-cyclopropa[f]indazol-3-yl]-1H-indole-5-carboxylate (X_{51c}) (1.5 g, 4.197 mmol, 1 equiv) in THF/MeOH/H₂O (5 mL/5 mL/5 mL) was added NaOH (1.6 g, 41.970 mmol, 10 equiv) at 0° C. The resulting mixture was stirred at 65° C. overnight. The product was precipitated by the addition of HCl. The product was purified by reverse phase column chromatography, eluted with CH₃CN/H₂O (35/65) to afford 2-[(4aS,5aR)-5,5-difluoro-5a-methyl-1H,4H,4aH,6H-cyclopropa[f]indazol-3-yl]-1H-indole-5-carboxylic acid (BBX₅₁) (700 mg, 48.3%) as a brown solid. MS (ESI) calc'd for (C₁₈H₁₅F₂N₃O₂) [M+1]⁺, 344.1; found, 344.1. ¹H NMR (400 MHz, DMSO-d₆) δ 11.64 (s, 1H), 8.20-7.71 (m, 1H), 7.71-7.69 (m, 1H), 7.68-7.42 (m, 1H), 7.42-5.10 (m, 1H), 3.20-2.96 (m, 3H), 2.86-2.82 (m, 1H), 1.89-1.84 (m, 1H), 1.39 (s, 3H).

### Example 141. 2-[(4aS,5aR)-5,5-Difluoro-5a-methyl-1H,4H,4aH,6H-cyclopropa[f]indazol-3-yl]-5-(piperazine-1-carbonyl)-1H-indole (BBX₅₂)

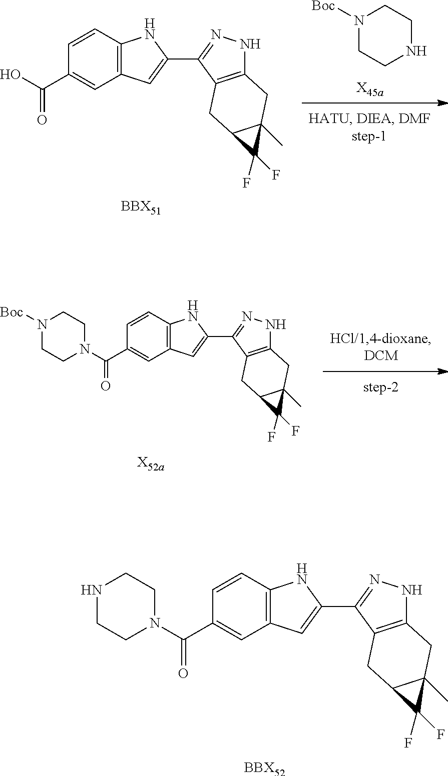

### Step-1: Synthesis of tert-Butyl 4-{2-[(4aS,5aR)-5,5-difluoro-5a-methyl-1H,4H,4aH,6H-cyclopropa[f]indazol-3-yl]-1H-indole-5-carbonyl}piperazine-1-carboxylate (X₅₂ₐ)

To a mixture of 2-[(4aS,5aR)-5,5-difluoro-5a-methyl-1H,4H,4aH,6H-cyclopropa[f]indazol-3-yl]-1H-indole-5-carboxylic acid (BBX₅₁) (450 mg, 1.311 mmol, 1 equiv) and tert-butyl piperazine-1-carboxylate (X₄₅ₐ) (366.1 mg, 1.966 mmol, 1.5 equiv) in DMF (5 mL) were added HATU (747.5 mg, 1.966 mmol, 1.5 equiv) and DIEA (508.2 mg, 3.933 mmol, 3 equiv) at room temperature. The resulting mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The residue was purified by C18 reverse phase column with CH₃CN/H₂O (35/65). This resulted in tert-butyl 4-{2-[(4aS,5aR)-5,5-difluoro-5a-methyl-1H,4H,4aH,6H-cyclopropa[f]indazol-3-yl]-1H-indole-5-carbonyl}piperazine-1-carboxylate (X₅₂ₐ) (230 mg, 34.30%) as a light yellow solid.MS (ESI) calc'd for (C₂₇H₃₁F₂N₅O₃) [M+1]⁺, 512.2; found, 512.2.

### Step-2: Synthesis of 2-[(4aS,5aR)-5,5-Difluoro-5a-methyl-1H,4H,4aH,6H-cyclopropa[f]indazol-3-yl]-5-(piperazine-1-carbonyl)-1H-indole (BBX₅₂)

To a mixture of tert-butyl 4-{2-[(4aS,5aR)-5,5-difluoro-5a-methyl-1H,4H,4aH,6H-cyclopropa[f]indazol-3-yl]-1H-indole-5-carbonyl}piperazine-1-carboxylate (X₅₂ₐ) (200 mg, 0.391 mmol, 1 equiv) in DCM (2 mL) was added HCl (gas) in 1,4-dioxane (2 mL, 4 N) at room temperature. The resulting mixture was stirred for 1 h at room temperature. The resulting mixture was concentrated under reduced pressure, then lyophilized. This resulted in 2-[(4aS,5aR)-5,5-difluoro-5a-methyl-1H,4H,4aH,6H-cyclopropa[f]indazol-3-yl]-5-(piperazine-1-carbonyl)-1H-indole (BBX₅₂) (121.8 mg, 74.89%) as a yellow solid. MS (ESI) calc'd for (C₂₂H₂₃F₂N₅O) [M+1]⁺, 412.2; found, 412.2. ¹H NMR (400 MHz, DMSO-d₆) δ 11.57 (d, J=2.0 Hz, 1H), 9.36 (s, 2H), 7.67 (s, 1H), 7.44 (d, J=8.4 Hz, 1H), 7.20-7.18 (m, 1H), 6.94-5.51 (m, 1H), 3.76-3.68 (m, 4H), 3.23-3.08 (m, 5H), 3.03-2.99 (m, 2H), 2.92-2.81 (m, 1H), 1.89-1.84 (m, 1H), 1.39 (s, 3H).

### Example 142. 6-(Methoxymethyl)-6-methyl-1,4,5,7-tetrahydroindazole-3-carboxylic acid (BBX₅₃)

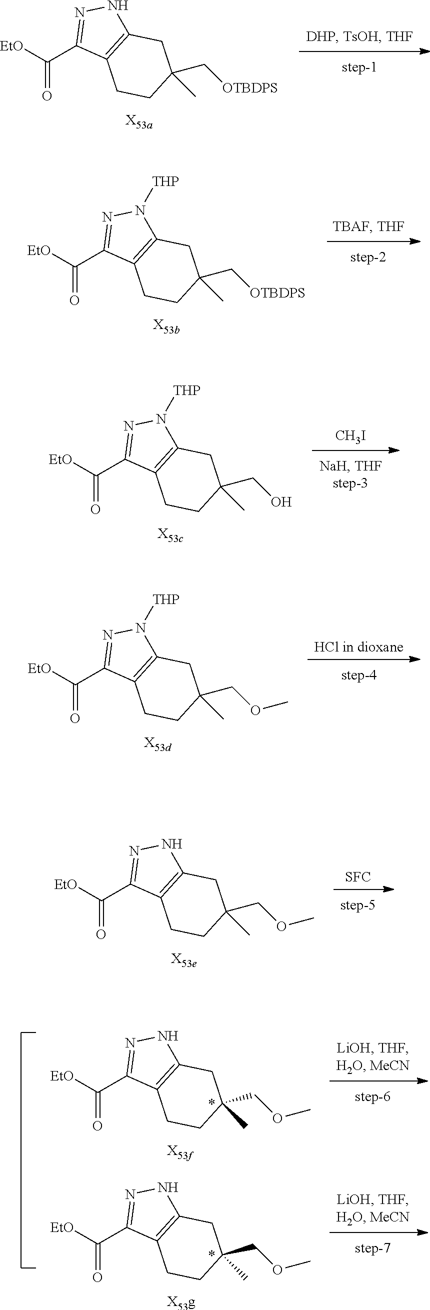

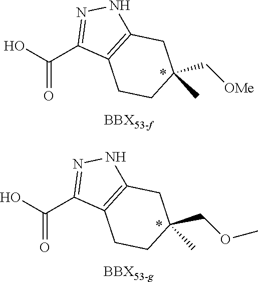

### Step 1: Synthesis of ethyl 6-{[(tert-butyldiphenylsilyl)oxy]methyl}-6-methyl-1-(oxan-2-yl)-5,7-dihydro-4H-indazole-3-carboxylate (X₅₃b)

To a mixture of ethyl 6-{[(tert-butyldiphenylsilyl)oxy]methyl}-6-methyl-1,4,5,7-tetrahydroindazole-3-carboxylate (X₅₃ₐ) (3 g, 6.293 mmol, 1 equiv) and DHP (1.06 g, 12.586 mmol, 2 equiv) in THF (30 mL) were added TsOH (541.86 mg, 3.147 mmol, 0.5 equiv) at room temperature. The resulting mixture was stirred for additional 2 h at room temperature. The resulting mixture was concentrated under reduced pressure. The residue was purified by silica gel column chromatography, eluted with PE/EA (7/1) to afford ethyl 6-{[(tert-butyldiphenylsilyl)oxy]methyl}-6-methyl-1-(oxan-2-yl)-5,7-dihydro-4H-indazole-3-carboxylate (X₅₃ₙ) (2.4 g, 62.56%) as a yellow oil. MS (ESI) calc'd for (C₃₃H₄₄N₂O₄Si) [M+1]⁺, 561.3; found, 561.0.

### Step 2: Synthesis of ethyl 6-(hydroxymethyl)-6-methyl-1-(oxan-2-yl)-5,7-dihydro-4H-indazole-3-carboxylate (X_{53c})

To a mixture of ethyl 6-{[(tert-butyldiphenylsilyl)oxy]methyl}-6-methyl-1-(oxan-2-yl)-5,7-dihydro-4H-indazole-3-carboxylate (X₅₃ₙ) (2.4 g, 4.28 mmol, 1 equiv) in THE (30 mL) was added TBAF (4.48 g, 17.12 mmol, 4 equiv) at room temperature. The resulting mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The residue was purified by silica gel column chromatography, eluted with PE/EA (1/1) to afford ethyl 6-(hydroxymethyl)-6-methyl-1-(oxan-2-yl)-5,7-dihydro-4H-indazole-3-carboxylate (X_{53c}) (1.5 g, 97.84%) as a yellow oil. MS (ESI) calc'd for (C₁₇H₂₆N₂O₄) [M+1]⁺, 323.2; found, 323.2.

Step 3: Ethyl 6-(methoxymethyl)-6-methyl-1-(oxan-2-yl)-5,7-dihydro-4H-indazole-3-carboxylate (X₅₃ₐ)

To a mixture of NaH (0.41 g, 17.214 mmol, 1.5 equiv) in THE (30 mL) was added ethyl 6-(hydroxymethyl)-6-methyl-1-(oxan-2-yl)-5,7-dihydro-4H-indazole-3-carboxylate (X_{53c}) (3.7 g, 11.47 mmol, 1 equiv) at 0° C. under nitrogen atmosphere. The resulting mixture was stirred for 1 h at room temperature. Then was added methyl iodide (2.4 g, 17.21 mmol, 1.5 equiv) at 0° C. The resulting mixture was stirred for 2 h at room temperature. The reaction was quenched with water at 0° C. The resulting mixture was extracted with EtOAc. The combined organic layers were washed with water, dried over anhydrous Na₂SO₄. After filtration, the filtrate was concentrated under reduced pressure. The residue was purified by silica gel column chromatography, eluted with PE/EA (1/1) to afford ethyl 6-(methoxymethyl)-6-methyl-1-(oxan-2-yl)-5,7-dihydro-4H-indazole-3-carboxylate (X₅₃ₐ) (1 g, 25.90%) as a yellow oil. MS (ESI) calc'd for (C₁₈H₂NN₂O₄) [M+1]⁺, 337.2; found, 337.2.

### Step 4: Synthesis of ethyl 6-(methoxymethyl)-6-methyl-1,4,5,7-tetrahydroindazole-3-carboxylate (X₅₃ₑ)

A mixture of ethyl 6-(methoxymethyl)-6-methyl-1-(oxan-2-yl)-5,7-dihydro-4H-indazole-3-carboxylate (X₅₃ₐ) (760 mg, 2.25 mmol, 1 equiv) in HCl (gas)/1,4-dioxane (5 mL, 4 N) was stirred for 1 h at room temperature. The resulting mixture was concentrated under reduced pressure to afford ethyl 6-(methoxymethyl)-6-methyl-1,4,5,7-tetrahydroindazole-3-carboxylate (X₅₃ₑ) (500 mg, crude) as a yellow oil. MS (ESI) calc'd for (C₁₃H₂₀N₂O₃) [M+1]⁺, 253.1; found, 253.2.

### Step 5: Synthesis of ethyl (6R)-6-(methoxymethyl)-6-methyl-1,4,5,7-tetrahydroindazole-3-carboxylate and ethyl (6S)-6-(methoxymethyl)-6-methyl-1,4,5,7-tetrahydroindazole-3-carboxylate (X_{53f}) (*Arbitrarily Assigned)

The racemic product X₅₃ₑ (300 mg) was separated by Chiral-HPLC with the following conditions (Column: CHIRAL ART Cellulose-SC, 2*25 cm, 5 m; Mobile Phase A: Hex(0.5% 2M NH₃-MeOH)-HPLC, Mobile Phase B: IPA-HPLC; Flow rate: 20 mL/min; Gradient: 8% B to 8% B in 20 min; Wave Length: 220/254 nm; RT1(min): 15.55; RT2(min): 18.29; Sample Solvent: MeOH:DCM=1:1-HPLC) to afford ethyl (6R)-6-(methoxymethyl)-6-methyl-1,4,5,7-tetrahydroindazole-3-carboxylate (X_{53f}) (100 mg, 33.33%) as a white solid. MS (ESI) calc'd for (C₁₃H₂₀N₂O₃) [M+1]⁺, 253.1; found, 253.2. and ethyl (6S)-6-(methoxymethyl)-6-methyl-1,4,5,7-tetrahydroindazole-3-carboxylate (X_{53g}) (160 mg, 53.33%) as a white solid. MS (ESI) calc'd for (C₁₃H₂₀N₂O₃) [M+1]⁺, 253.2; found, 253.1.

### Step 6: Synthesis of (6R)-6-(Methoxymethyl)-6-methyl-1,4,5,7-tetrahydroindazole-3-carboxylic acid (BBX₅₃-f) (*Arbitrarily Assigned)

To a mixture of ethyl (6R)-6-(methoxymethyl)-6-methyl-1,4,5,7-tetrahydroindazole-3-carboxylate (X_{53f}) (100 mg, 0.04 mmol, 1 equiv) in THF (2 mL), MeCN (2 mL) and H₂O (2 mL) was added LiGH (199.5 mg, 4.7 mmol, 12 equiv). The resulting mixture was stirred for 3 h at 50° C. The mixture was acidified to pH=3 with HCl (2 N) aqueous. The precipitated solids were collected by filtration and washed with water to afford (6R)-6-(methoxymethyl)-6-methyl-1,4,5,7-tetrahydroindazole-3-carboxylic acid (BBX₅₃-f) (99.0 mg, crude) as a white solid. MS (ESI) calc'd for (C₁₁H₁₆N₂O₃) [M+1]⁺, 225.2; found, 225.0. ¹H NMR (400 MHz, Methanol-d₄) δ 3.37-3.33 (m, 3H), 3.24-3.21 (m, 2H), 2.82-2.73 (m, 2H), 2.71-2.60 (m, 1H), 2.41-2.37 (m, 1H), 1.75-1.66 (m, 1H), 1.65-1.49 (m, 1H), 1.01 (s, 3H).

### Step 7: Synthesis of (6S)-6-(Methoxymethyl)-6-methyl-1,4,5,7-tetrahydroindazole-3-carboxylic acid (BBX_{53-g}) (*Arbitrarily Assigned)

To a mixture of ethyl (6S)-6-(methoxymethyl)-6-methyl-1,4,5,7-tetrahydroindazole-3-carboxylate (X_{53g}) (160 mg, 0.04 mmol, 1 equiv) in THE (2 mL), MeCN (2 mL) and H₂O (2 mL) was added LiGH (319.3 mg, 7.6 mmol, 12 equiv) The resulting mixture was stirred for 3 h at 50° C. The mixture was acidified to pH=3 with HCl (2 N) aqueous. The precipitated solids were collected by filtration and washed with water to afford (6S)-6-(methoxymethyl)-6-methyl-1,4,5,7-tetrahydroindazole-3-carboxylic acid (BBX_{53-g}) (141.4 mg, crude) as a white solid. MS (ESI) calc'd for (C₁₁H₁₆N₂O₃) [M+1]⁺, 225.2; found, 225.0. ¹H NMR (400 MHz, Methanol-d₄) δ 3.37-3.32 m, 3H), 3.30-3.21 (m, 2H), 2.82-2.73 (m, 2H), 2.68-2.60 (m, 1H), 2.41-2.37 (m, 1H), 1.75-1.65 (m, 1H), 1.60-1.51 (m, 1H), 1.01 (s, 3H).

The following General Procedures Schemes 1-14 illustrate the bond formations by which the CRBN harnesses may be coupled with the ITK hooks to afford the CRBN-based ITK CTMs of name and structure.

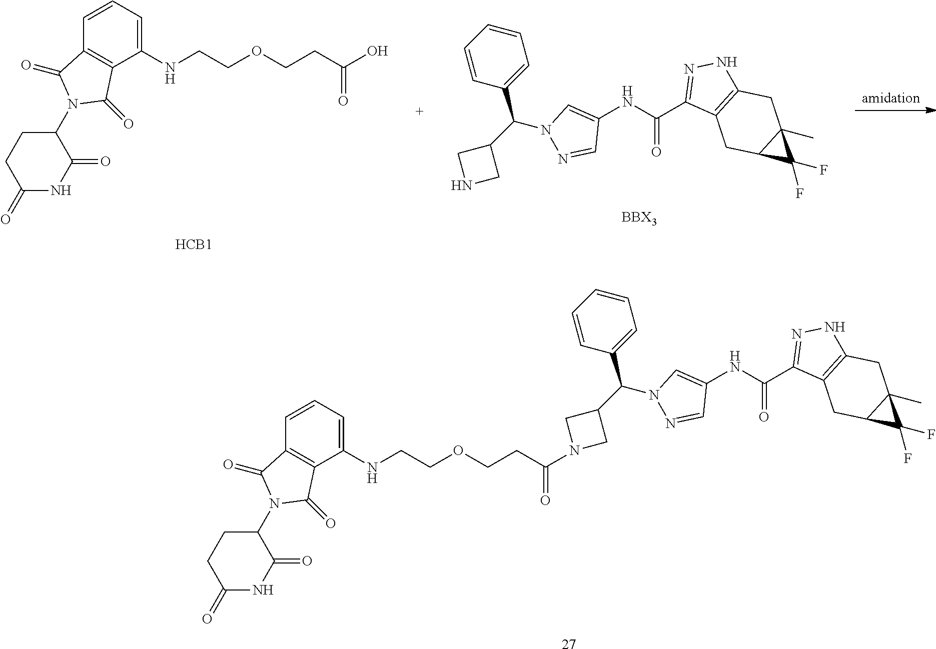

### Scheme 1: Synthesis of Compound 27 Via Amide Formation

A mixture of amine (15 mg, 0.03 mmol), carboxylic acid (20 mg, 0.05 mmol), HATU (10 mg, 0.05 mmol), and i-Pr₂NEt (18 μL, 0.10 mmol) in DMF (200 μL) was allowed to stir at room temperature for one hour. The mixture was purified by HPLC (H₂O:MeCN with 0.1% TFA) to afford the amide product (14 mg, 0.0175 mmol, 51%). An exemplary amide coupling is provided in Scheme 1 above where (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₃) was treated with 3-(2-((2-(2,6-dioxopiperidin-3-yl)-1,3-dioxoisoindolin-4-yl)amino)ethoxy)propanoic acid (HCB1) as described above to provide (4aS,5aR)-N-(1-((1S)-(1-(3-(2-((2-(2,6-dioxopiperidin-3-yl)-1,3-dioxoisoindolin-4-yl)amino)ethoxy)propanoyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (Compound 27).

HATU or BOP was typically used as a coupling reagent, but other coupling agents have been contemplated as would be appreciated by a person having skill in the art.

Other amide containing compounds of this disclosure that were synthesized using General Procedure 1 are Compounds 28-30, 32, 36-40, 42, 43, 49-51, 55, 56, 81, 82, and 88-91.

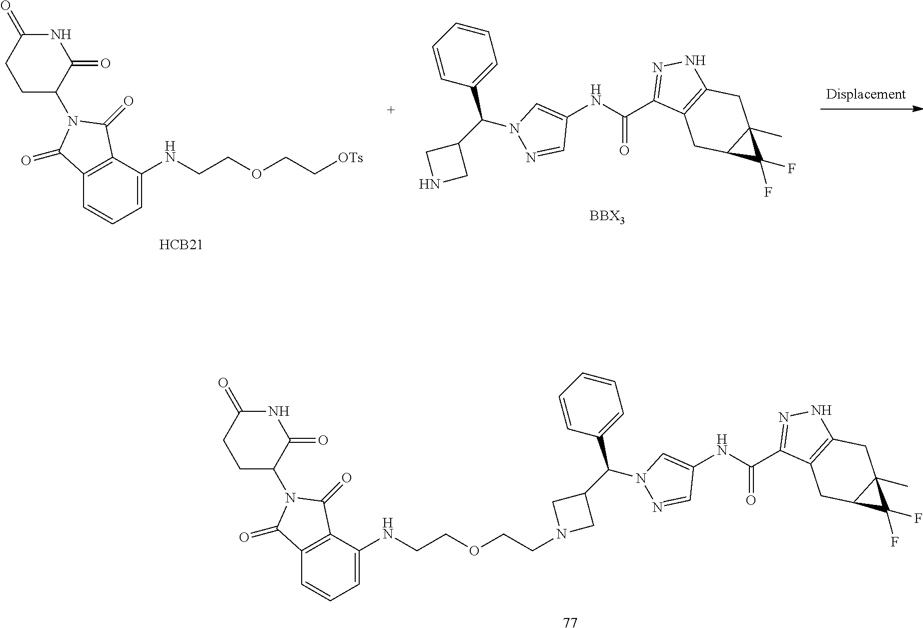

### Scheme 2: Synthesis of Compound 77 via Displacement

A mixture of amine (20 mg, 0.05 mmol), tosylate (24 mg, 0.05 mmol), and i-Pr₂NEt (23.7 μL, 0.14 mmol) in DMF (200 μL) was stirred at 70° C. for one hour. Purification via HPLC (20-70% MeCN:H₂O) afforded the amine product (5 mg, 0.01 mmol, 14%). An exemplary displacement is provided in Scheme 2 above where (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₃) was treated with 2-(2-((2-(2,6-dioxopiperidin-3-yl)-1,3-dioxoisoindolin-4-yl)amino)ethoxy)ethyl 4-methylbenzenesulfonate (HCB21) as described above to provide (4aS,5aR)-N-(1-((1S)-(1-(2-(2-((2-(2,6-dioxopiperidin-3-yl)-1,3-dioxoisoindolin-4-yl)amino)ethoxy)ethyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (Compound 77).

Another amine containing compound of this disclosure that was synthesized by using General Procedure 2 is Compound 31.

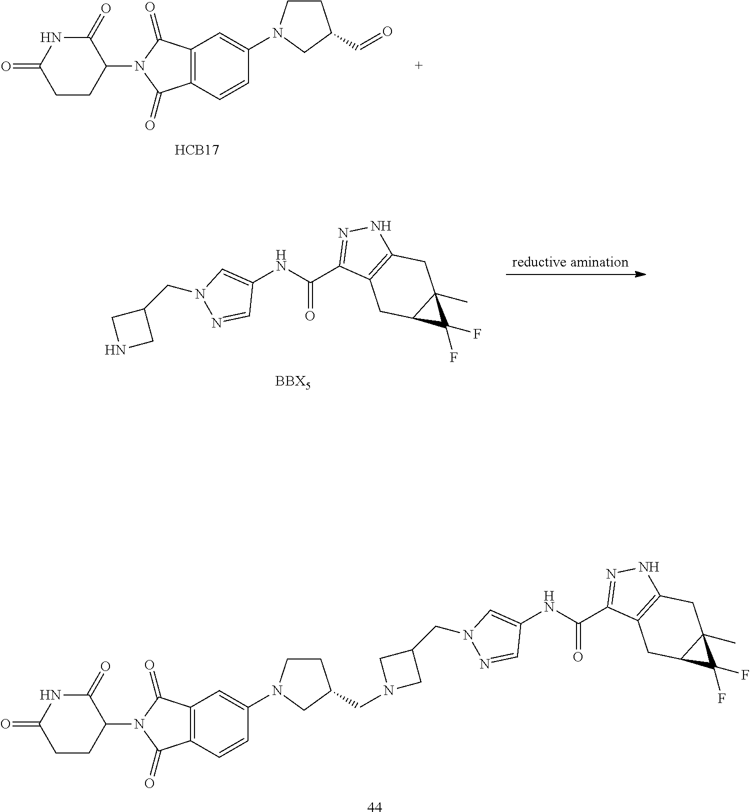

### Scheme 3: Synthesis of Compound 44 via Reductive Amination

A mixture of amine (39 mg, 0.11 mmol), aldehyde (38 mg, 0.11 mmol), and Et₃N (75 μL, 0.54 mmol) in DCE (1.1 mL) was allowed to stir at rt for 10 min. NaBH(OAc)₃ (68 mg, 0.32 mmol) was added, and the reaction mixture was allowed to stir at rt for one hour. The reaction was then quenched with H₂O, extracted with DCM, concentrated, and purified by HPLC (H₂O:MeCN with 0.1% TFA) to afford the amine product (11 mg, 0.01 mmol, 13%). An exemplary reductive amination is provided in Scheme 3 where (4aS,5aR)-N-(1-(azetidin-3-ylmethyl)-1H-pyrazol-4-yl)-5,5-difluoro-5a-methyl-1,4,4a,5,5a,6-hexahydrocyclopropafindazole-3-carboxamide (BBX₅) was treated with (3S)-1-(2-(2,6-dioxopiperidin-3-yl)-1,3-dioxoisoindolin-5-yl)pyrrolidine-3-carbaldehyde (HCB17) as described above to provide (4aS,5aR)-N-(1-((1-(((3R)-1-(2-(2,6-dioxopiperidin-3-yl)-1,3-dioxoisoindolin-5-yl)pyrrolidin-3-yl)methyl)azetidin-3-yl)methyl)-1H-pyrazol-4-yl)-5, 5-difluoro-5a-methyl-1,4,4a,5,5a,6-hexahydrocyclopropa[f]indazole-3-carboxamide (Compound 44). DCE or DCM was typically used as a solvent. Et₃N or i-Pr₂NEt was typically used as a base.

Other amine containing compounds of this disclosure that were synthesized by using General Procedure 3 are Compounds 41, and 57.

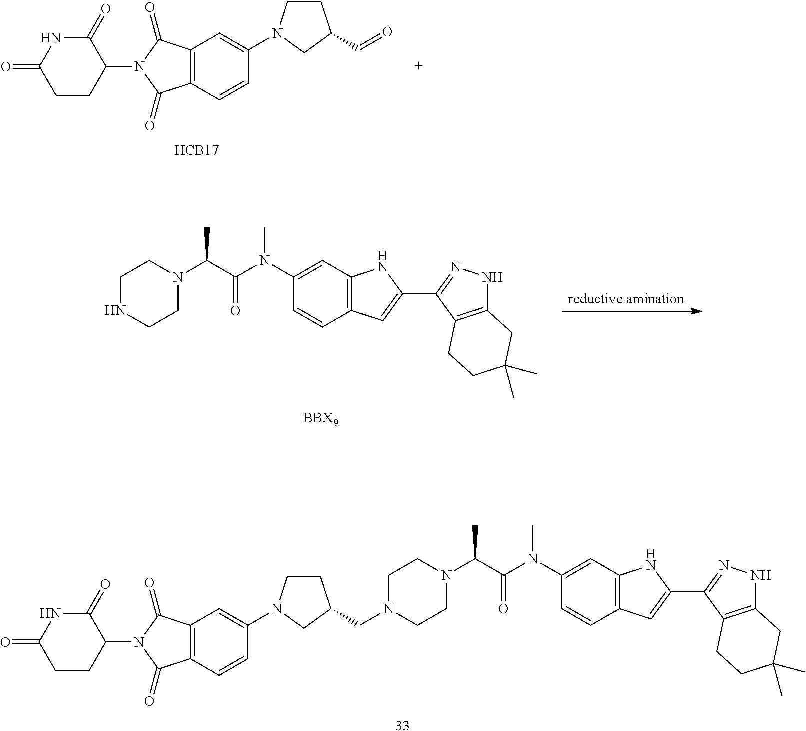

### Scheme 4: Synthesis of Compound 37 Via Reductive Amination

A mixture of amine (16 mg, 0.04 mmol), aldehyde (13 mg, 0.04 mmol), and Et₃N (26 μL, 0.18 mmol) in DCE (0.4 mL) was allowed to stir at rt for 10 min. NaBH(OAc)₃ (23 mg, 0.11 mmol) was added, and the reaction mixture was allowed to stir at rt for one hour. The reaction was quenched with H₂O, extracted with DCM, concentrated, and purified by HPLC (H₂O:MeCN with 0.1% TFA) to afford the amine product (19 mg, 0.02 mmol, 62%). An exemplary reductive amination is provided in Scheme 4 where (S)—N-(2-(6,6-dimethyl-4,5,6,7-tetrahydro-1H-indazol-3-yl)-1H-indol-6-yl)-N-methyl-2-(piperazin-1-yl)propanamide (BBX₉) was treated with (3S)-1-(2-(2,6-dioxopiperidin-3-yl)-1,3-dioxoisoindolin-5-yl)pyrrolidine-3-carbaldehyde (HCB17) as described above to provide (2S)—N-(2-(6,6-dimethyl-4,5,6,7-tetrahydro-1H-indazol-3-yl)-1H-indol-6-yl)-2-(4-(((3R)-1-(2-(2,6-dioxopiperidin-3-yl)-1,3-dioxoisoindolin-5-yl)pyrrolidin-3-yl)methyl)piperazin-1-yl)-N-methylpropanamide (Compound 37).

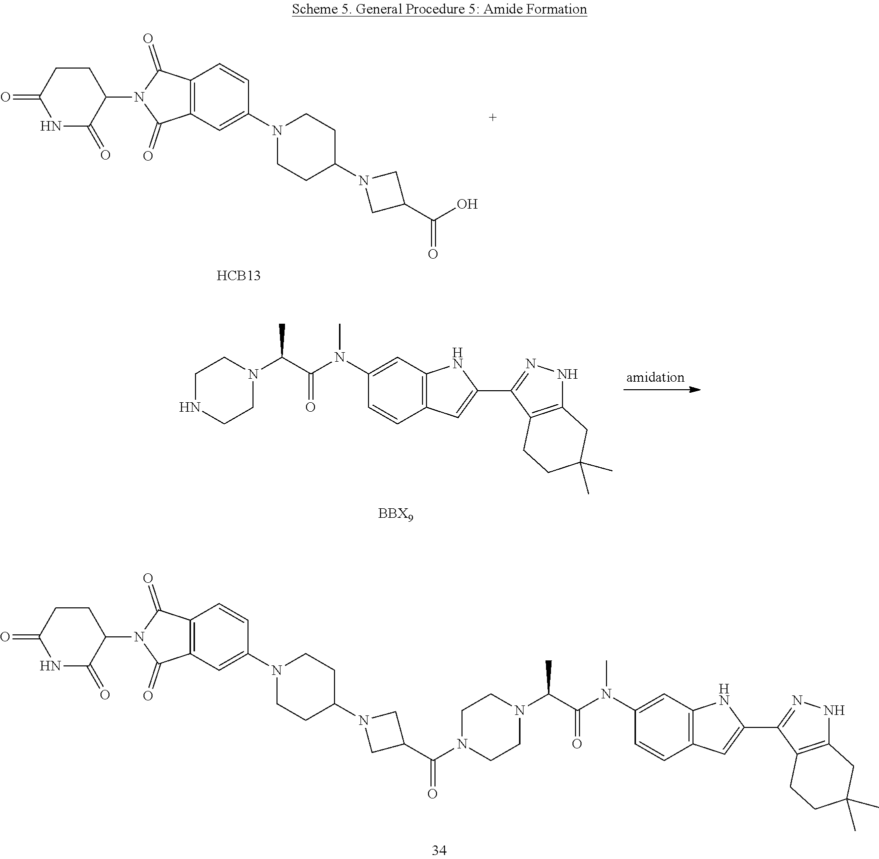 [SMILES: O=C1CCC(N2C(=O)c3ccc(N4CCC(N5CC(C(=O)O)C5)CC4)cc3C2=O)C(=O)N1 ; "HCB13" ; C[C@@H](C(=O)N(C)c1ccc2cc(-c3n[nH]c4c3CCC(C)(C)C4)[nH]c2c1)N1CCNCC1 ; "+" ; C[C@@H](C(=O)N(C)c1ccc2cc(-c3n[nH]c4c3CCC(C)(C)C4)[nH]c2c1)N1CCN(C(=O)C2CN(C3CCN(c4ccc5c(c4)C(=O)N(C4CCC(=O)NC4=O)C5=O)CC3)C2)CC1]

### Scheme 5: Synthesis of Compound 34 via Amide Formation

A mixture of amine (20 mg, 0.050 mmol), carboxylic acid (20 mg, 0.050 mmol), BOP (24 mg, 0.05 mmol), and i-Pr₂NEt (39 μL, 0.23 mmol) in DMF (200 μL) was allowed to stir at room temperature for 0.5 h. The reaction mixture was purified by HPLC (H₂O:MeCN with 0.1% TFA) to afford the amide product (9.4 mg, 0.01 mmol, 24%). An exemplary amide coupling is provided in Scheme 5 above where (S)—N-(2-(6,6-dimethyl-4,5,6,7-tetrahydro-1H-indazol-3-yl)-1H-indol-6-yl)-N-methyl-2-(piperazin-1-yl)propanamide (BBX₉) was treated with 1-(1-(2-(2,6-dioxopiperidin-3-yl)-1,3-dioxoisoindolin-5-yl)piperidin-4-yl)azetidine-3-carboxylic acid (HCB13) as described above to provide (2S)—N-(2-(6,6-dimethyl-4,5,6,7-tetrahydro-1H-indazol-3-yl)-1H-indol-6-yl)-2-(4-(1-(1-(2-(2,6-dioxopiperidin-3-yl)-1,3-dioxoisoindolin-5-yl)piperidin-4-yl)azetidine-3-carbonyl)piperazin-1-yl)-N-methylpropanamide (Compound 34).

HATU or BOP was typically used as a coupling reagent, but other coupling agents have been contemplated as would be appreciated by a person having skill in the art.

Another amide containing compound of this disclosure that was synthesized by using General Procedure 5 is Compound 48.

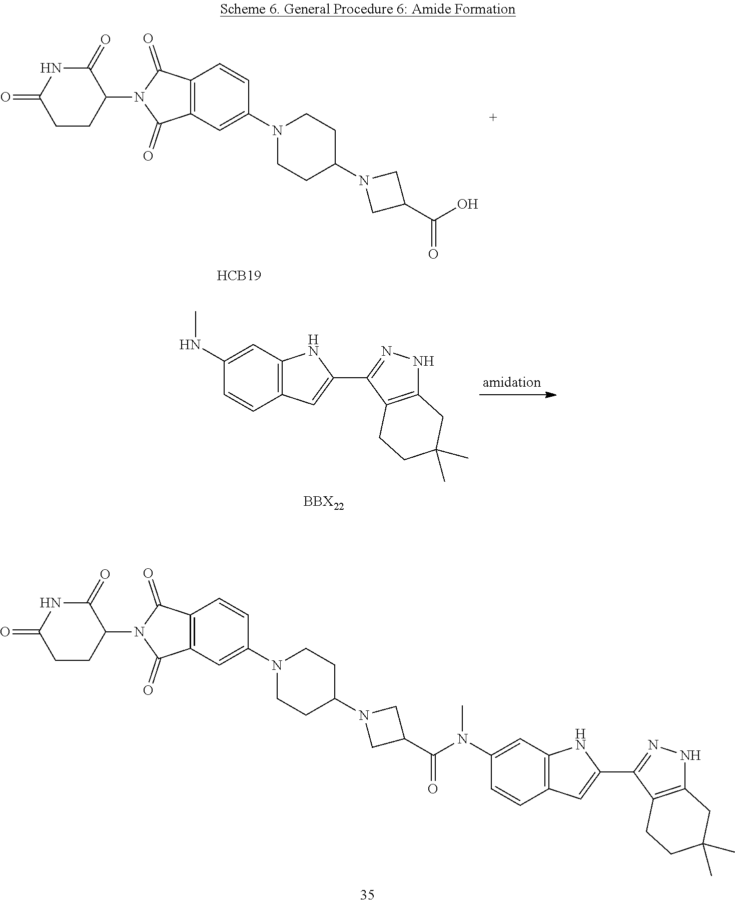

### Scheme 6: Synthesis of Compound 35 via Amide Formation

A mixture of amine (20 mg, 0.070 mmol), carboxylic acid (30 mg, 0.070 mmol), BOP (36 mg, 0.08 mmol), and i-Pr₂NEt (59 μL, 0.34 mmol) in DMF (350 μL) was allowed to stir at room temperature for 0.5 h. The reaction mixture was purified by HPLC (H₂O:MeCN with 0.1% TFA) to afford the amide product (8.4 mg, 0.01 mmol, 17%). An exemplary amide coupling is provided in Scheme 6 above where 2-(6,6-dimethyl-4,5,6,7-tetrahydro-1H-indazol-3-yl)-N-methyl-1H-indol-6-amine (BBX₂₂) was treated with 1-(1-(2-(2,6-dioxopiperidin-3-yl)-1,3-dioxoisoindolin-5-yl)piperidin-4-yl)azetidine-3-carboxylic acid (HCB19) as described above to provide N-(2-(6,6-dimethyl-4,5,6,7-tetrahydro-1H-indazol-3-yl)-1H-indol-6-yl)-1-(1-(2-(2,6-dioxopiperidin-3-yl)-1,3-dioxoisoindolin-5-yl)piperidin-4-yl)-N-methylazetidine-3-carboxamide (Compound 35). BBX₂₂ was prepared consistent with WO 2011/065402, the content of which is incorporated herein by reference in its entirety.

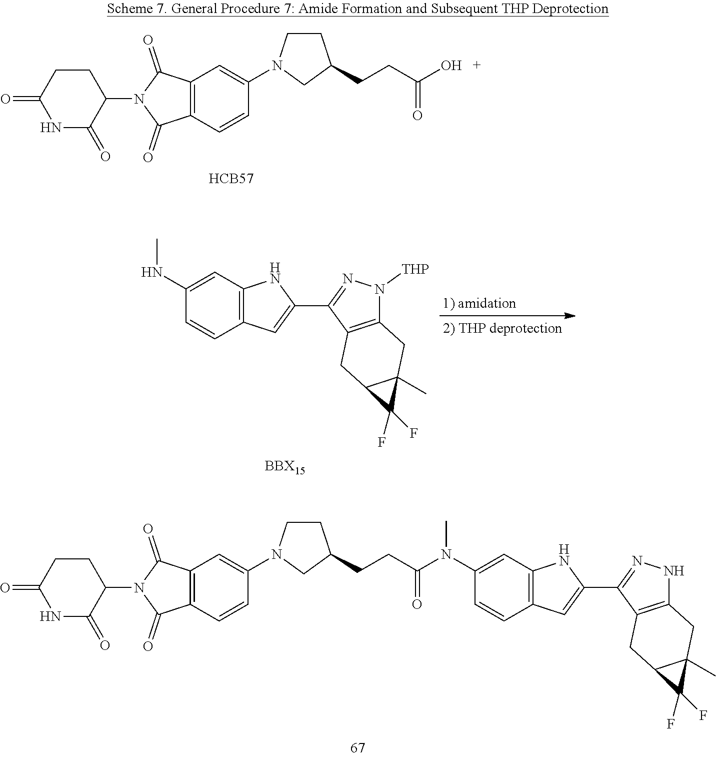

### Synthesis of 3-((3R)-1-(2-(2,6-dioxopiperidin-3-yl)-1,3-dioxoisoindolin-5-yl)pyrrolidin-3-yl)propanoic acid (HCB57)

Followed the procedure described for Example 24 (HCB51 synthesis) starting from (R)-3-(pyrrolidin-3-yl)propanoic acid (3 g, 21 mmol, crude) and 2-(2,6-dioxopiperidin-3-yl)-5-fluoroisoindoline-1,3-dione (3.5 g, 13 mmol) to afford the desired product (4.4 g, 11 mmol, 50%). LCMS: C₂₀H₂₁N₃O₆ requires 399, found: m/z=400 [M+H]⁺.

### Scheme 7: Synthesis of Compound 67 via Amide Formation and Subsequent THP Deprotection

To a mixture of carboxylic acid (110 mg, 0.275 mmol), HATU (150 mg, 0.396 mmol), and i-Pr₂NEt (102 mg, 0.793 mmol) in DMF (3 mL) was added amine (109 mg, 0.264 mmol). The reaction mixture was stirred at rt for 16 h, quenched with H₂O, and extracted with EtOAc. The combined organic layers were washed with H₂O, dried over MgSO₄, and concentrated under reduced pressure. Reverse phase flash chromatography (C18, MeCN:H₂O) afforded the amide product (120 mg, 57%).

HATU, T3P, or BOP was typically used as a coupling reagent, but other coupling agents have been contemplated as would be appreciated by a person having skill in the art.

A mixture of tetrahydropyranyl ether (140 mg, 0.176 mmol, 1 equiv) and HCl (4 M in dioxane, 8 mL) was stirred at room temperature for one hour. The resulting mixture was concentrated under vacuum. The crude product (60 mg) was purified by Prep-HPLC under the following conditions: Column: Xselect CSH OBD Column 30*150 mm 5 μm; Mobile Phase A: Water (0.1% formic acid), Mobile Phase B: MeCN; Flow rate: 60 mL/min; Gradient: 48% B to 56% B in 8 min, 56% B; Wave Length: 254/220 nm to afford the desired product (12 mg, 9.59% yield) as a yellow solid. An exemplary amide coupling is provided in Scheme 7 above where 2-((4aS,5aR)-5,5-difluoro-5a-methyl-1-(tetrahydro-2H-pyran-2-yl)-1,4,4a,5,5a,6-hexahydrocyclopropa[f]indazol-3-yl)-N-methyl-1H-indol-6-amine (BBX₁₅) was treated with 3-((3R)-1-(2-(2,6-dioxopiperidin-3-yl)-1,3-dioxoisoindolin-5-yl)pyrrolidin-3-yl)propanoic acid (HCB57) as described above to provide N-(2-((4aS,5aR)-5,5-difluoro-5a-methyl-1,4,4a,5,5a,6-hexahydrocyclopropafindazol-3-yl)-1H-indol-6-yl)-3-((3R)-1-(2-(2,6-dioxopiperidin-3-yl)-1,3-dioxoisoindolin-5-yl)pyrrolidin-3-yl)-N-methylpropanamide (Compound 67).

Other amide containing compounds of this disclosure that was synthesized by using General Procedure 7 are Compounds 1, 2, 20, 21, 36, 58, 61, 69, and 70.

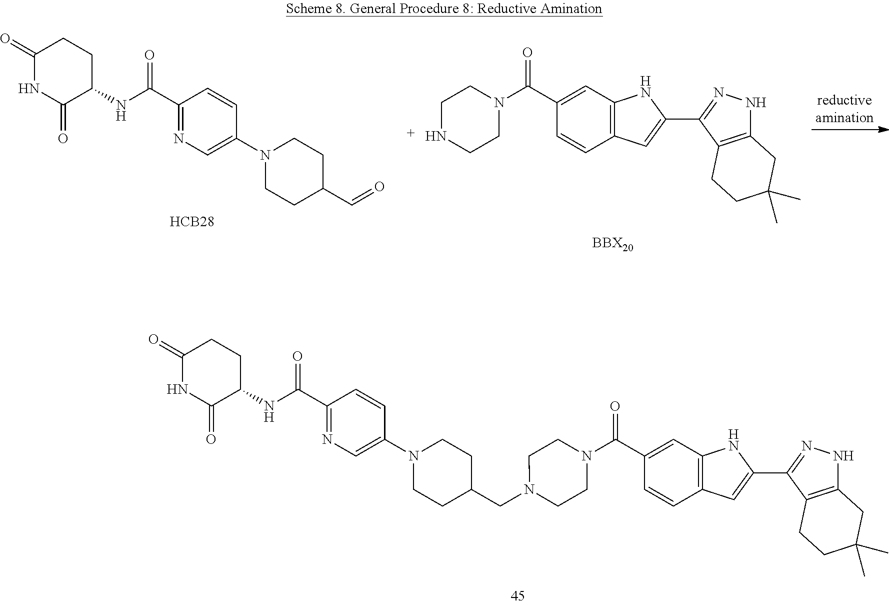

### Scheme 8: Synthesis of Compound 45 via Reductive Amination

A mixture of amine (25 mg, 0.07 mmol), aldehyde (24 mg, 0.07 mmol), and Et₃N (45 μL, 0.18 mmol) in DCE (0.4 mL) was allowed to stir at rt for 10 min. NaBH(OAc)₃ (23 mg, 0.33 mmol) was added, and the reaction mixture was allowed to stir at rt for one hour. The reaction was quenched with H₂O, extracted with DCM, concentrated, and purified by HPLC (H₂O:MeCN with 0.1% TFA) to afford the amine product (6.1 mg, 0.01 mmol, 12%). An exemplary reductive amination is provided in Scheme 8 where (2-(6,6-dimethyl-4,5,6,7-tetrahydro-1H-indazol-3-yl)-1H-indol-6-yl)(piperazin-1-yl)methanone (BBX₂₀) was treated with (S)—N-(2,6-dioxopiperidin-3-yl)-5-(4-formylpiperidin-1-yl)picolinamide (HCB28) as described above to provide (S)-5-(4-((4-(2-(6,6-dimethyl-4,5,6,7-tetrahydro-1H-indazol-3-yl)-1H-indole-6-carbonyl)piperazin-1-yl)methyl)piperidin-1-yl)-N-(2,6-dioxopiperidin-3-yl)picolinamide (45). ¹H NMR (500 MHz, (CD₃)₂SO) δ 11.54 (s, 1H), 10.86 (d, J=4.0 Hz, 2H), 9.42 (s, 1H), 8.71 (dd, J=8.3, 4.2 Hz, 2H), 8.33 (dd, J=16.4, 2.9 Hz, 2H), 7.87 (t, J=8.9 Hz, 2H), 7.58 (d, J=8.1 Hz, 1H), 7.55 (s, 1H), 7.43 (ddd, J=21.1, 9.1, 2.9 Hz, 2H), 7.10 (d, J=8.0 Hz, 1H), 6.66 (s, 1H), 4.83 (d, J=5.4 Hz, 1H), 4.75 (ddd, J=13.1, 7.9, 5.3 Hz, 2H), 3.99 (t, J=13.9 Hz, 3H), 3.12 (s, 4H), 2.87 (dt, J=42.7, 13.0 Hz, 4H), 2.69 (t, J=6.5 Hz, 2H), 2.55 (s, 2H), 2.43 (s, 2H), 2.18 (qd, J=12.8, 4.1 Hz, 1H), 2.06-1.99 (m, 1H), 1.83 (dd, J=22.9, 12.8 Hz, 4H), 1.59 (t, J=6.3 Hz, 2H), 1.48-1.28 (m, 3H), 1.02 (s, 6H). LCMS: C₃₉H₄₇N₉O₄ requires: 706, found: m/z=707 [M+H]⁺. DCE or DCM was typically used as a solvent. Et₃N or i-Pr₂NEt was typically used as a base.

Other amine containing compounds of this disclosure that were synthesized by using General Procedure 8 are Compounds 26, 46-48 52, and 60.

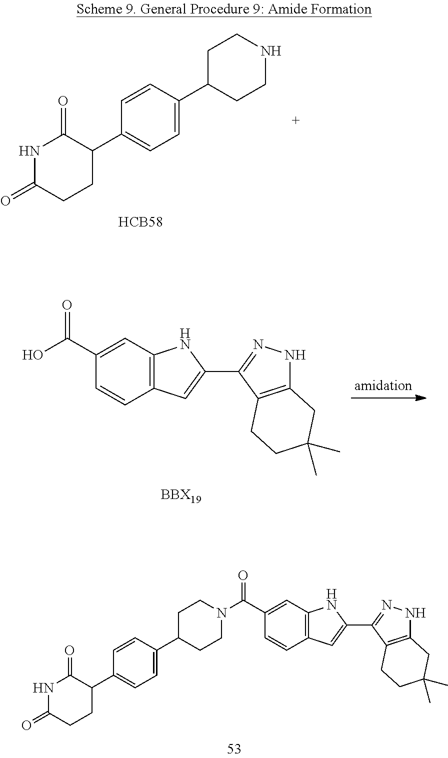 [SMILES: "HCB58" ; O=C1CCC(c2ccc(C3CCNCC3)cc2)C(=O)N1 ; CC1(C)CCc2c(-c3cc4ccc(C(=O)O)cc4[nH]3)n[nH]c2C1 ; "+" ; CC1(C)CCc2c(-c3cc4ccc(C(=O)N5CCC(c6ccc(C7CCC(=O)NC7=O)cc6)CC5)cc4[nH]3)n[nH]c2C1]

### Scheme 9: Synthesis of Compound 53 via Amide Formation

A mixture of amine (29 mg, 0.11 mmol), carboxylic acid (40 mg, 0.13 mmol), BOP (57 mg, 0.13 mmol), and i-Pr₂NEt (93 μL, 0.54 mmol) in DMF (550 μL) was allowed to stir at room temperature for 16 h. The reaction mixture was purified by HPLC (H₂O:MeCN with 0.1% TFA) to afford the amide product (20 mg, 0.04 mmol, 33%). An exemplary amide coupling is provided in Scheme 9 above where 3-(4-(piperidin-4-yl)phenyl)piperidine-2,6-dione (HCB58) was treated with 2-(6,6-dimethyl-4,5,6,7-tetrahydro-1H-indazol-3-yl)-1H-indole-6-carboxylic acid (BBX₁₉) as described above to provide 3-(4-(1-(2-(6,6-dimethyl-4,5,6,7-tetrahydro-1H-indazol-3-yl)-1H-indole-6-carbonyl)piperidin-4-yl)phenyl)piperidine-2,6-dione (Compound 53). ¹H NMR (500 MHz, (CD3)2SO) δ 11.40 (s, 1H), 10.82 (s, 1H), 7.54 (d, J=8.1 Hz, 1H), 7.49 (s, 1H), 7.25 (d, J=7.9 Hz, 2H), 7.16 (d, J=7.9 Hz, 2H), 7.05 (dd, J=8.1, 1.5 Hz, 1H), 6.62 (d, J=2.1 Hz, 1H), 3.82 (dd, J=11.5, 5.0 Hz, 1H), 2.82 (tt, J=12.4, 3.6 Hz, 1H), 2.71-2.60 (m, 3H), 2.46 (t, J=4.2 Hz, OH), 2.41 (s, 2H), 2.17 (ddt, J=16.0, 11.8, 5.8 Hz, 1H), 2.03 (dq, J=13.8, 4.9 Hz, 1H), 1.81 (s, 2H), 1.65 (dd, J=12.2, 4.0 Hz, 1H), 1.59 (dt, J=12.9, 5.2 Hz, 3H), 1.01 (s, 6H). LCMS: C₃₄H₃₇N₅O₃ requires: 563, found: m/z=564 [M+H]⁺.

HATU or BOP was typically used as a coupling reagent, but other coupling agents have been contemplated as would be appreciated by a person having skill in the art.

Other amine containing compounds of this disclosure that were synthesized by using General Procedure 9 are Compounds 37, 54, 58, 59, and 61-63.

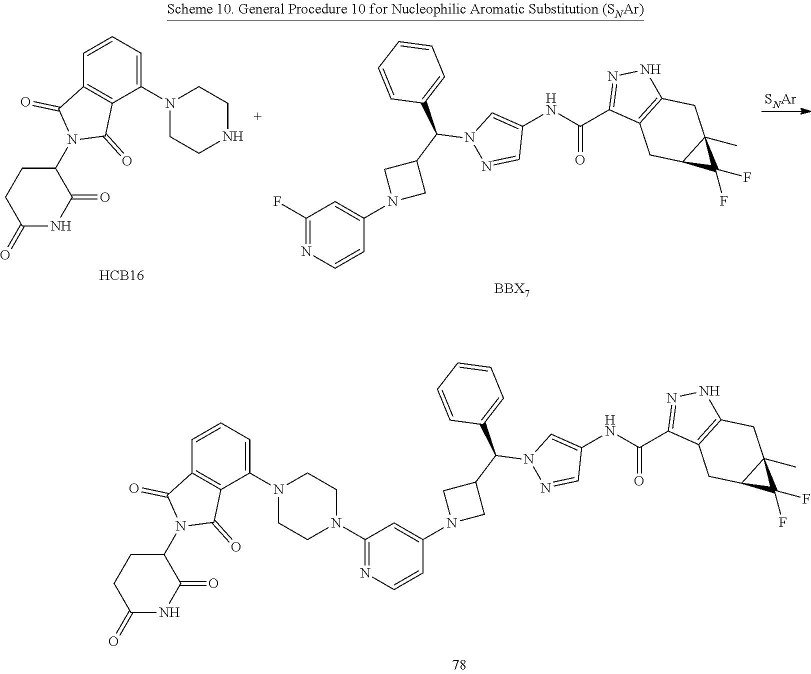

### Scheme 10: Synthesis of Compound 78 via S_{N}Ar

2-(2,6-dioxopiperidin-3-yl)-4-(piperazin-1-yl)isoindole-1,3-dione (HCB16) (138.25 mg, 0.4 mmol) and (4aS,5aR)-5,5-difluoro-N-{1-[(S)-[1-(2-fluoropyridin-4-yl)azetidin-3-yl](phenyl)methyl]pyrazol-4-yl}-5a-methyl-1H,4H,4aH,6H-cyclopropa[f]indazole-3-carboxamide (BBX₇) (106 mg, 0.2 mmol) were dissolved in dimethyl sulfoxide (1.01 mL, 1.11 g, 14.21 mmol), and triethylamine (137.7 L, 0.1 g, 0.99 mmol) was added dropwise. The reaction was transferred to a 0.5-2 mL microwave vial and heated to 120° C. in a microwave reactor for 7.5 h. An additional equivalent of triethylamine (27.5 L, 0.02 g, 0.2 mmol) was added and the reaction was heated to 120° C. in a microwave reactor for another 10 h. The reaction was diluted with dichloromethane and washed with water. The crude material was concentrated and purified by reverse phase-HPLC (5-95% MeCN in water) to provide (4aS,5aR)-N-{1-[(S)-[1-(2-{4-[2-(2,6-dioxopiperidin-3-yl)-1,3-dioxoisoindol-4-yl]piperazin-1-yl}pyridin-4-yl)azetidin-3-yl](phenyl)methyl]pyrazol-4-yl}-5,5-difluoro-5a-methyl-1H,4H,4aH,6H-cyclopropa[f]indazole-3-carboxamide (Compound 78) (4 mg, 2%) as a yellow solid. LCMS: C₄₅H₄₃F₂NiO₅ requires: 855.3, found: m/z=856.7 [M+H]⁺.

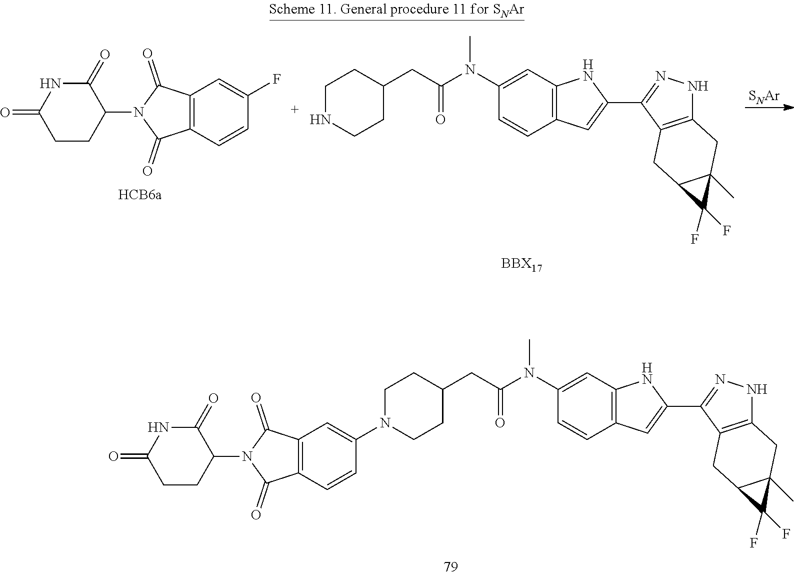

### Scheme 11: Synthesis of Compound 79 via SNAr

A mixture of rac-2-[(3R)-2,6-dioxopiperidin-3-yl]-5-fluoroisoindole-1,3-dione (HCB6a) (14.4 mg, 0.05 mmol), i-Pr₂NEt (28 μL, 20.78 mg, 0.16 mmol), and N-{2-[(4aS,5aR)-5,5-difluoro-5a-methyl-1H,4H,4aH,6H-cyclopropa[indazol-3-yl]-1H-indol-6-yl}-N-methyl-2-(piperidin-4-yl)acetamide (22.68 mg, 0.05 mmol) (BBX₁₇) was dissolved in NMP (1.00 mL, 1.03 g, 10.39 mmol) and heated to 65° C. for 2 h. The reaction was then cooled, concentrated, and purified by flash chromatography on a 4 g column, eluting with 0 to 20% MeOH:DCM to provide N-{2-[(4aS,5aR)-5,5-difluoro-5a-methyl-1H,4H,4aH,6H-cyclopropa[ ]indazol-3-yl]-1H-indol-6-yl}-2-(1-{2-[(3RS)-2,6-dioxopiperidin-3-yl]-1,3-dioxoisoindol-5-yl}piperidin-4-yl)-N-methylacetamide (Compound 79) (9.3 g, 25%) as a yellow solid. LCMS: C₃₈H₃₇F₂N₇O₅ requires: 709.3, found: m/z=710.5 [M+H]⁺.

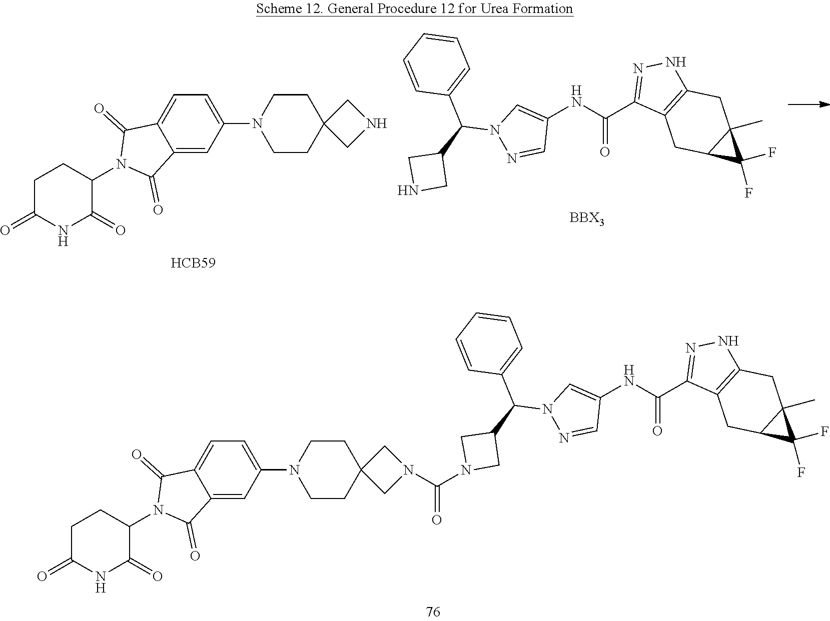 [SMILES: O=C1CCC(N2C(=O)c3ccc(N4CCC5(CC4)CNC5)cc3C2=O)C(=O)N1 ; "HCB59" ; C[C@@]12Cc3[nH]nc(C(=O)Nc4cnn([C@H](c5ccccc5)C5CN(C(=O)N6CC7(CCN(c8ccc9c(c8)C(=O)N(C8CCC(=O)NC8=O)C9=O)CC7)C6)C5)c4)c3C[C@@H]1C2(F)F ; C[C@@]12Cc3[nH]nc(C(=O)Nc4cnn([C@H](c5ccccc5)C5CNC5)c4)c3C[C@@H]1C2(F)F]

### Synthesis of 5-{2,7-diazaspiro[3.5]nonan-7-yl}-2-(2,6-dioxopiperidin-3-yl)isoindole-1,3-dione (HCB59)

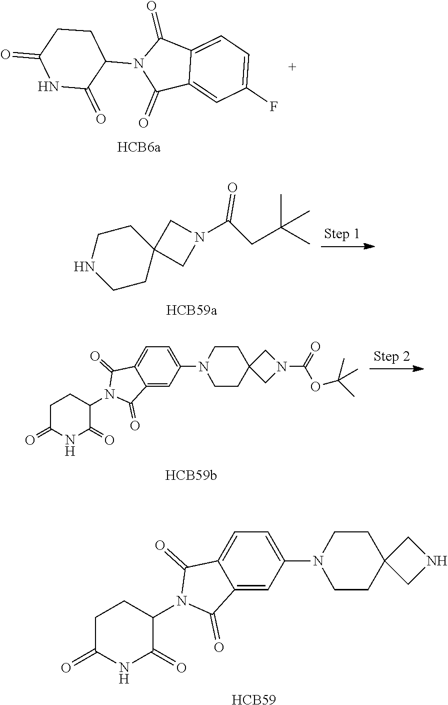

Step-1: To a solution of 2-(2,6-dioxopiperidin-3-yl)-5-fluoroisoindole-1,3-dione (75.00 mg, 0.27 mmol) and tert-butyl 2,7-diazaspiro[3.5]nonane-2-carboxylate (61.45 mg, 0.27 mmol) in 2 ml NMP was added N, N-diisopropylethylamine (142 ul, 0.81 mmol), the mixture was heated at 85° C. overnight. The solution was cooled down. ISCO silica gel column purification eluting with EtOAc in hexane (5-100%) to afford desired product (68 mg, 0.14 mmol, 51.9%). tert-butyl 7-[2-(2,6-dioxopiperidin-3-yl)-1,3-dioxoisoindol-5-yl]-2,7-diazaspiro[3.5]nonane-2-carboxylate. LCMS: C₂₅H₃₀N₄O₆ requires: 483, found: m/z=484 [M+H]⁺.

Step-2: A solution of tert-butyl 7-[2-(2,6-dioxopiperidin-3-yl)-1,3-dioxoisoindol-5-yl]-2,7-diazaspiro[3.5]nonane-2-carboxylate (68.00 mg, 0.14 mmol) in 1 ml DCM was added 1 ml 4N HCl in dioxane, the solution was stirred for 45 minutes. The volatiles were evaporated to provide desired product. The crude product was used to the next step without further purification. 5-{2,7-diazaspiro[3.5]nonan-7-yl}-2-(2,6-dioxopiperidin-3-yl)isoindole-1,3-dione (HCB59) LCMS: C₂₀H₂₂N₄O₄ requires: 383, found: m/z=384 [M+H]⁺.

### Scheme 12: Synthesis of Compound 76 via Urea Formation

To (4aS,5aR)-N-{1-[(S)-azetidin-3-yl(phenyl)methyl]pyrazol-4-yl}-5,5-difluoro-5a-methyl-1H,4H,4aH,6H-cyclopropafindazole-3-carboxamide (BBX₃) (8.00 mg, 0.02 mmol) in DCM (1 mL), was added trimethylamine (TEA) (5 μL, 0.04 mmol) and the solution was cooled in an ice-bath under nitrogen. Then 4-nitrophenyl chloroformate (3.68 mg, 0.02 mmol) in DCM (0.5 mL) was added and the reaction was stirred for 15 minutes. The product was washed with water, dried over Na₂SO₄, and concentrated. ISCO silica gel column (4 g) chromatography provided pure intermediate. The intermediate was dissolved in NMP (0.5 mL) and 5-{2,7-diazaspiro[3.5]nonan-7-yl}-2-(2,6-dioxopiperidin-3-yl)isoindole-1,3-dione (HCB59) (6.98 mg, 0.02 mmol) in NMP (0.5 mL) and triethylamine (5.06 uL, 3.69 mg, 0.04 mmol) were each added, and the reaction was stirred at 65° C. overnight. The crude product was purified by Prep-HPLC to afford (4aS,5aR)-N-{1-[(S)-(1-{7-[2-(2,6-dioxopiperidin-3-yl)-1,3-dioxoisoindol-5-yl]-2,7-diazaspiro[3.5]nonane-2-carbonyl}azetidin-3-yl)(phenyl)methyl]pyrazol-4-yl}-5,5-difluoro-5a-methyl-1H,4H,4aH,6H-cyclopropa[f]indazole-3-carboxamide (Compound 76) (4.1 mg, 26.5% yield). ¹H NMR (500 MHz, DMSO-d₆) δ 12.96 (s, 1H), 11.08 (s, 1H), 10.17 (s, 1H), 8.08 (s, 1H), 7.66 (d, J=2.8 Hz, 1H), 7.42-7.28 (m, 6H), 7.22 (dd, J=8.7, 2.3 Hz, 1H), 5.65 (d, J=10.1 Hz, 1H), 5.07 (dd, J=12.8, 5.4 Hz, 1H), 4.04 (s, 7H), 3.86 (dt, J=43.0, 8.0 Hz, 2H), 3.68 (dt, J=13.5, 6.4 Hz, 1H), 3.06 (d, J=5.1 Hz, 2H), 2.10-1.96 (m, 1H), 1.83-1.66 (m, 5H), 1.47 (q, J=6.7 Hz, 2H), 1.35 (s, 3H), 1.32-1.11 (m, 6H), 0.91-0.66 (m, 2H). LCMS: C₄₄H₄₄F₂N₁₀O₆ requires: 846, found: m/z=847 [M+H]⁺.

Another amine containing compound of this disclosure that was synthesized by using General Procedure 12 is Compound 53.

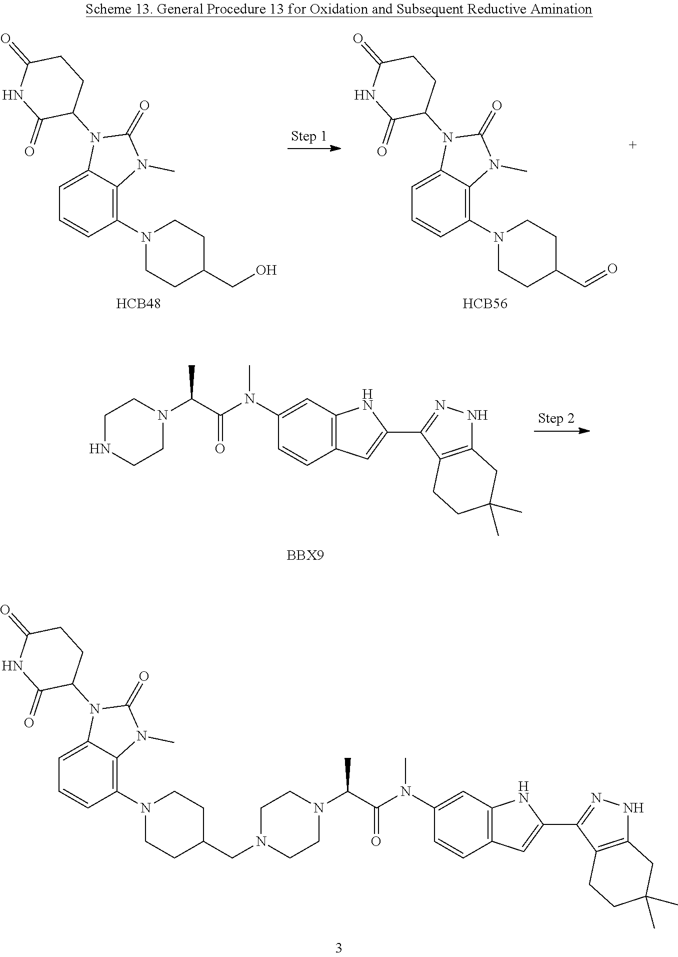

### Scheme 13: Synthesis of Compound 3 via Oxidation and Subsequent Reductive Amination

Sulfur trioxide pyridine complex (141 mg, 0.89 mmol) was added to a mixture of alcohol (33 mg, 0.09 mmol) and triethylamine (0.25 mL, 1.8 mmol) in DMSO (1.00 mL). The reaction mixture was allowed to stir for 20 min and was quenched with H₂O. The reaction mixture was extracted with DCM, and concentrated to afford the aldehyde product. An exemplary oxidation is provided in Scheme 13 where 3-(4-(4-(hydroxymethyl)piperidin-1-yl)-3-methyl-2-oxo-2,3-dihydro-1H-benzo[d]imidazol-1-yl)piperidine-2,6-dione (BBX₉) was oxidized as described above to provide a mixture containing 1-(1-(2,6-dioxopiperidin-3-yl)-3-methyl-2-oxo-2,3-dihydro-1H-benzo[d]imidazol-4-yl)piperidine-4-carbaldehyde (HCB56) and DMSO (Compound 3).

A mixture of amine (38.7 mg, 0.09 mmol), aldehyde (0.09 mmol), and N,N-diisopropylethylamine (0.06 mL, 0.36 mmol) in DCM (1 mL) was allowed to stir at rt for 10 min. NaBH(OAc)₃ (38 mg, 0.18 mmol) was added, and the reaction mixture was allowed to stir at rt for 0.5 h. The reaction was quenched with H₂O, extracted with DCM, concentrated, and purified by prep HPLC (10 to 95% MeCN in water with 0.1% TFA) to afford the amine product (0.0284 g, 38.8%). An exemplary reductive amination is provided in Scheme Xu where (2S)—N-[2-(6,6-dimethyl-1,4,5,7-tetrahydroindazol-3-yl)-1H-indol-6-yl]-N-methyl-2-(piperazin-1-yl)propanamide was treated with 1-(1-(2,6-dioxopiperidin-3-yl)-3-methyl-2-oxo-2,3-dihydro-1H-benzo[d]imidazol-4-yl)piperidine-4-carbaldehyde as described above to provide (2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-4-yl)piperidin-4-yl)methyl)piperazin-1-yl)-N-methylpropanamide (Compound 3).

SO₃·Pyr or DMP was typically used as an oxidant.

DCE or DCM was typically used as a solvent. Et₃N or i-Pr₂NEt was typically used as a base.

Other amine containing compounds of this disclosure that were synthesized by using General Procedure 13 are Compounds 10-14, 16-19, 22, 23, and 25.

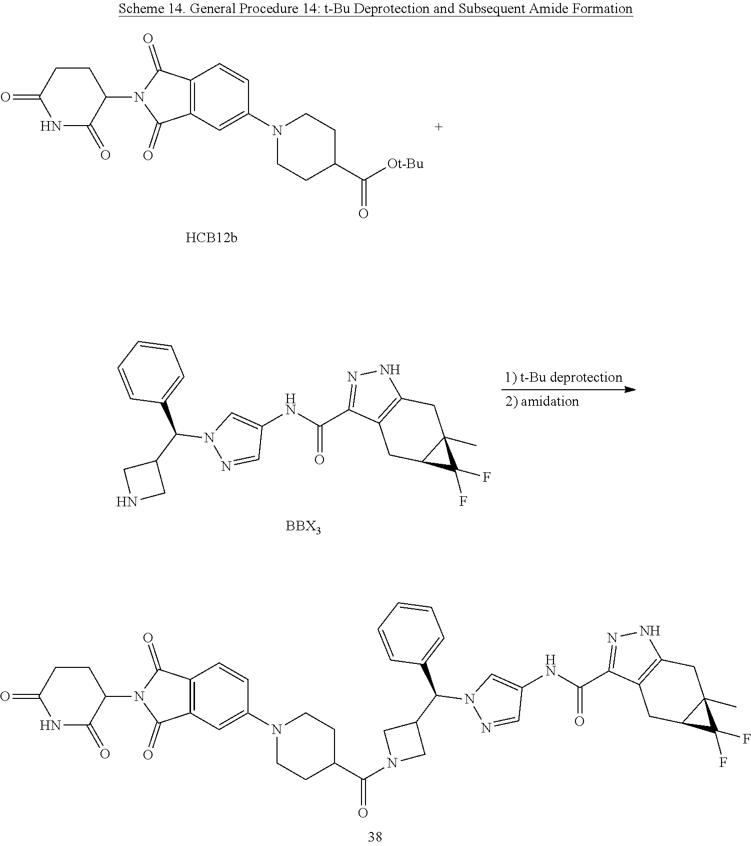 [SMILES: CC(C)(C)OC(=O)C1CCN(c2ccc3c(c2)C(=O)N(C2CCC(=O)NC2=O)C3=O)CC1 ; "+" ; C[C@@]12Cc3[nH]nc(C(=O)Nc4cnn([C@H](c5ccccc5)C5CNC5)c4)c3C[C@@H]1C2(F)F ; C[C@@]12Cc3[nH]nc(C(=O)Nc4cnn([C@H](c5ccccc5)C5CN(C(=O)C6CCN(c7ccc8c(c7)C(=O)N(C7CCC(=O)NC7=O)C8=O)CC6)C5)c4)c3C[C@@H]1C2(F)F]

Scheme 14: Synthesis of Compound 38 via t-Bu Deprotection and Subsequent Amide Formation

A mixture of t-Bu ester (40 mg, 0.09 mmol) and HCl (4 M in dioxane, 250 μL) was stirred at room temperature for 2 h. The resulting mixture was concentrated under reduced pressure. The crude product was used in the next step without purification.

To a mixture of the resulting carboxylic acid, BOP (48 mg, 0.11 mmol), and i-Pr₂NEt (157 mg, 0.91 mmol) in DMF (0.5 mL) was added amine (39.7 mg, 0.09 mmol). The reaction mixture was stirred at rt for 16 h, concentrated under reduced pressure, and purified by flash chromatography, eluting with 0 to 10% MeOH:DCM to provide the amide product (5.7 mg, 0.01 mmol, 7.4%). HATU or BOP was typically used as a coupling reagent, but other coupling agents have been contemplated as would be appreciated by a person having skill in the art.

An exemplary amide coupling is provided in Scheme 14 above where tert-butyl 1-(2-(2,6-dioxopiperidin-3-yl)-1,3-dioxoisoindolin-5-yl)piperidine-4-carboxylate (HCB12b) was treated with (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₃) as described above to provide (4aS,5aR)-N-(1-((1S)-(1-(1-(2-(2,6-dioxopiperidin-3-yl)-1,3-dioxoisoindolin-5-yl)piperidine-4-carbonyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (Compound 38).

Other amide containing compounds of this disclosure that were synthesized by using General Procedure 14 are Compound 36-38, and 40.

### General Procedure 15: Reductive Amination

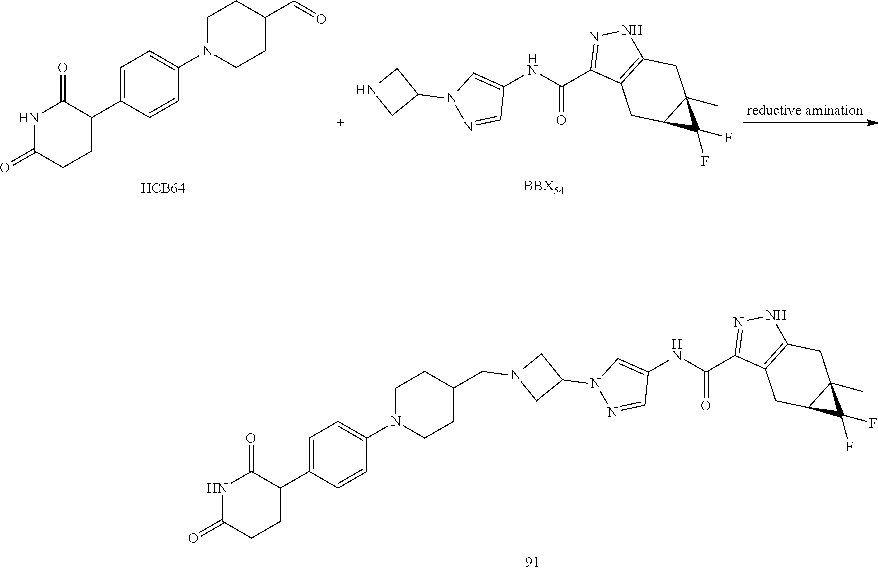

Scheme 15: Synthesis of Compound 91 via amine formation

A mixture of amine (30 mg, 0.086 mmol), aldehyde (28 mg, 0.095 mmol), i-Pr₂NEt (75 μL, 0.43 mmol) in DCM (0.9 mL) was allowed to stir at rt for 15 min. NaBH(OAc)₃ (55 mg, 0.26 mmol) was added, and the reaction mixture was allowed to stir at rt for 1.5 hr. The reaction was quenched with H₂O. The reaction mixture was extracted with 10% MeOH/DCM, and concentrated under reduced pressure. PTLC (SiO₂, 10% MeOH/DCM) provided the amine product (13 mg, 0.020 mmol, 23%). ¹H NMR (500 MHz, (CD₃)₂SO) δ 12.98 (s, 1H), 10.77 (s, 1H), 10.19 (s, 1H), 8.16 (s, 1H), 7.70 (s, 1H), 7.04 (d, J=8.3 Hz, 2H), 6.89 (d, J=8.4 Hz, 2H), 4.95 (p, J=6.9 Hz, 1H), 3.76-3.63 (m, 4H), 3.10-3.01 (m, 2H), 2.83 (d, J=17.3 Hz, 1H), 2.69-2.58 (m, 3H), 2.50-2.40 (m, 2H), 2.22-2.07 (m, 1H), 2.02 (dq, J=8.4, 4.9 Hz, 1H), 1.79 (d, J=12.3 Hz, 2H), 1.50-1.42 (m, 1H), 1.37 (s, 3H), 1.26 (d, J=7.2 Hz, 5H). LCMS: C₃₃H₃₈F₂N₈O₃ requires: 632, found: m/z=633 [M+H]⁺. An exemplary reductive amination is provided in Scheme 15 where (4aS,5aR)-N-(1-(azetidin-3-yl)-1H-pyrazol-4-yl)-5,5-difluoro-5a-methyl-1,4,4a,5,5a,6-hexahydrocyclopropa[f]indazole-3-carboxamide and 1-(4-(2,6-dioxopiperidin-3-yl)phenyl)piperidine-4-carbaldehyde were reacted as described above to provide (4aS,5aR)-N-(1-(1-((1-(4-(2,6-dioxopiperidin-3-yl)phenyl)piperidin-4-yl)methyl)azetidin-3-yl)-1H-pyrazol-4-yl)-5,5-difluoro-5a-methyl-1,4,4a,5,5a,6-hexahydrocyclopropa[f]indazole-3-carboxamide (91).

### General Procedure 16: Reductive Amination

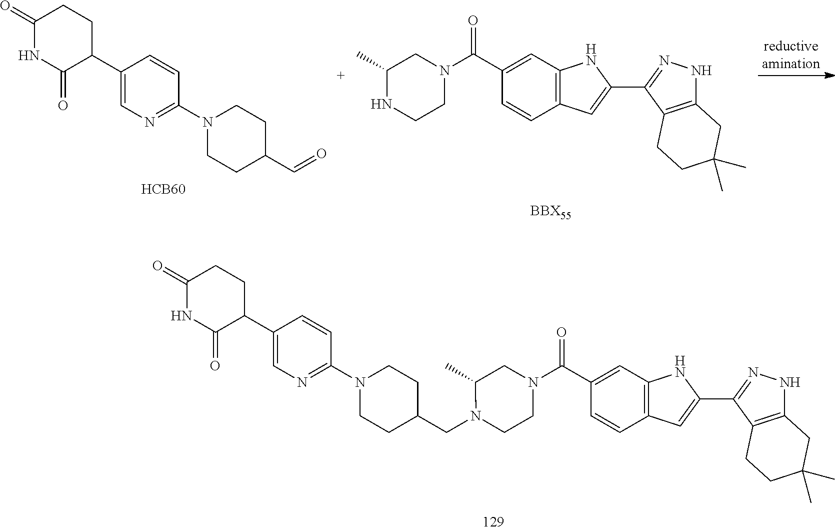

Scheme 16: Synthesis of Compound 129 via reductive amination

A mixture of amine (20 mg, 0.051 mmol), aldehyde (15 mg, 0.051 mmol), i-Pr₂NEt (89 μL, 0.51 mmol) in DCM (0.5 mL) was allowed to stir at rt for 15 min. NaBH(OAc)₃ (33 mg, 0.15 mmol) was added, and the reaction mixture was allowed to stir at rt for 1.5 h. The reaction was quenched with H₂O. The reaction mixture was extracted with 10% MeOH/DCM, and concentrated under reduced pressure. PTLC (SiO₂, 7.5% MeOH/DCM) provided the amine product (11 mg, 0.016 mmol, 31%). ¹H NMR (500 MHz, CDCl₃) δ 10.48 (s, 1H), 10.13 (s, 1H), 8.06 (d, J=2.5 Hz, 1H), 7.59 (d, J=8.2 Hz, 1H), 7.51 (s, 1H), 7.31 (dt, J=8.4, 4.0 Hz, 1H), 7.15-7.09 (m, 1H), 6.71 (d, J=2.1 Hz, 1H), 6.70-6.64 (m, 1H), 4.27 (d, J=14.8 Hz, 3H), 3.72-3.63 (m, 2H), 3.60-2.94 (m, 2H), 2.87 (d, J=12.7 Hz, 1H), 2.85-2.74 (m, 4H), 2.69 (ddd, J=17.2, 10.4, 5.4 Hz, 1H), 2.54 (s, 1H), 2.44 (s, 2H), 2.24 (qd, J=14.5, 11.8, 6.3 Hz, 2H), 1.94 (d, J=12.8 Hz, 1H), 1.76 (s, 1H), 1.65 (t, J=6.4 Hz, 2H), 1.27-1.17 (m, 3H), 1.05 (s, 6H). LCMS: C₃₉H₄₈N₈O₃ requires: 676, found: m/z=677 [M+H]⁺. An exemplary reductive amination is provided in Scheme 16 where (R)-(2-(6,6-dimethyl-4,5,6,7-tetrahydro-1H-indazol-3-yl)-1H-indol-6-yl)(3-methylpiperazin-1-yl)methanone and 1-(5-(2,6-dioxopiperidin-3-yl)pyridin-2-yl)piperidine-4-carbaldehyde were reacted as described above to provide 3-(6-(4-(((R)-4-(2-(6,6-dimethyl-4,5,6,7-tetrahydro-1H-indazol-3-yl)-1H-indole-6-carbonyl)-2-methylpiperazin-1-yl)methyl)piperidin-1-yl)pyridin-3-yl)piperidine-2,6-dione (129).

### Example 143. N-(2-(6,6-dimethyl-4,5,6,7-tetrahydro-1H-indazol-3-yl)-1H-indol-6-yl)-3-(1-(2-(2,6-dioxopiperidin-3-yl)-1,3-dioxoisoindolin-5-yl)pyrrolidin-3-yl)-N-methylpropanamide (1)

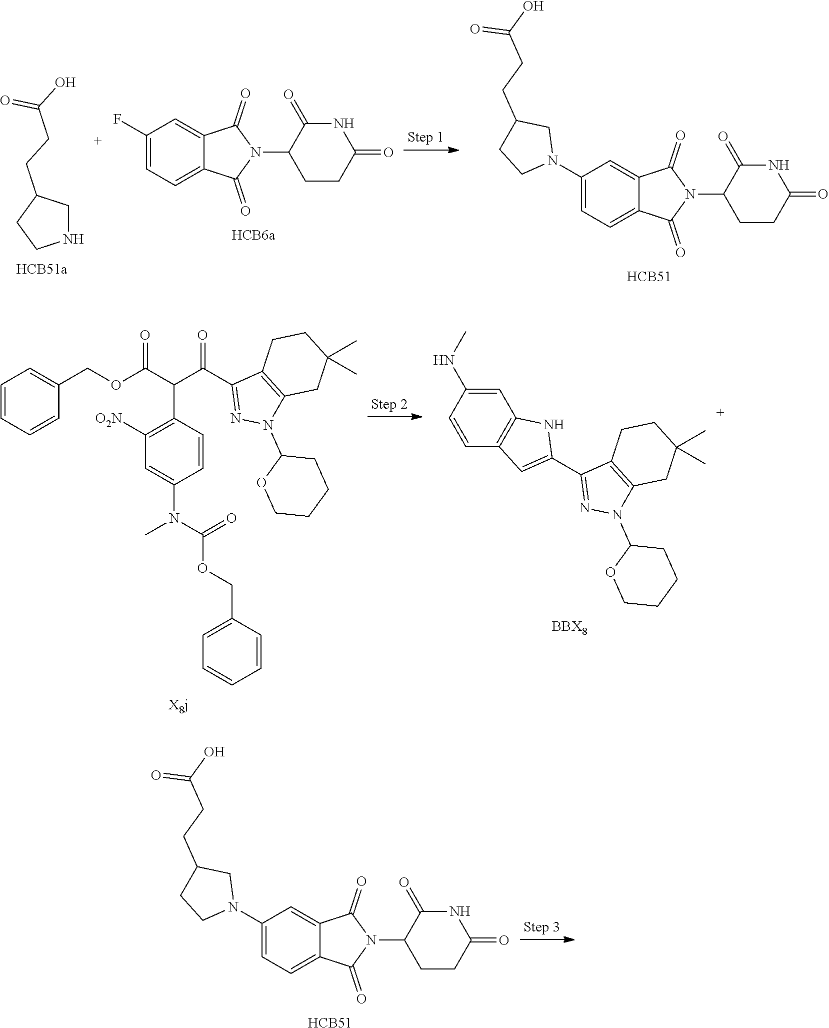

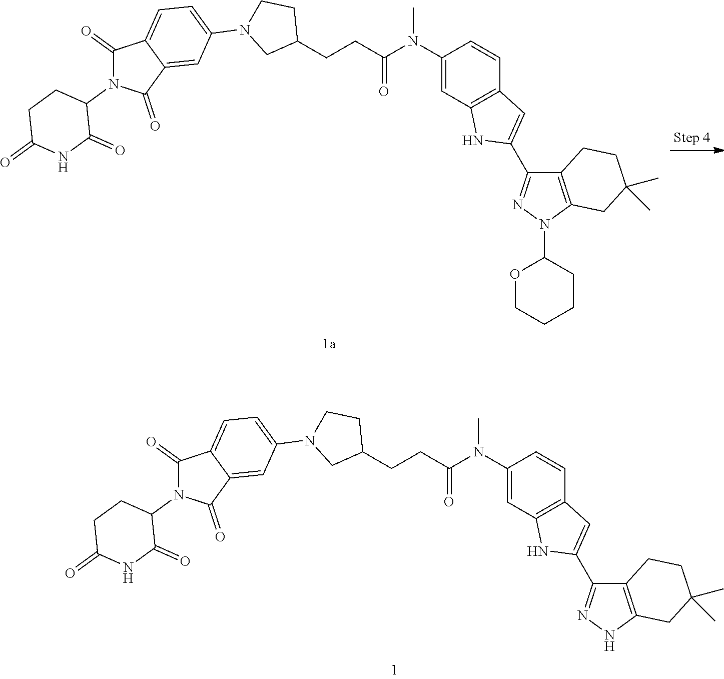 [SMILES: CN(C(=O)CCC1CCN(c2ccc3c(c2)C(=O)N(C2CCC(=O)NC2=O)C3=O)C1)c1ccc2cc(-c3nn(C4CCCCO4)c4c3CCC(C)(C)C4)[nH]c2c1 ; CN(C(=O)CCC1CCN(c2ccc3c(c2)C(=O)N(C2CCC(=O)NC2=O)C3=O)C1)c1ccc2cc(-c3n[nH]c4c3CCC(C)(C)C4)[nH]c2c1]

### Step 1: 3-(1-(2-(2,6-dioxopiperidin-3-yl)-1,3-dioxoisoindolin-5-yl)pyrrolidin-3-yl)propanoic acid (HCB51)

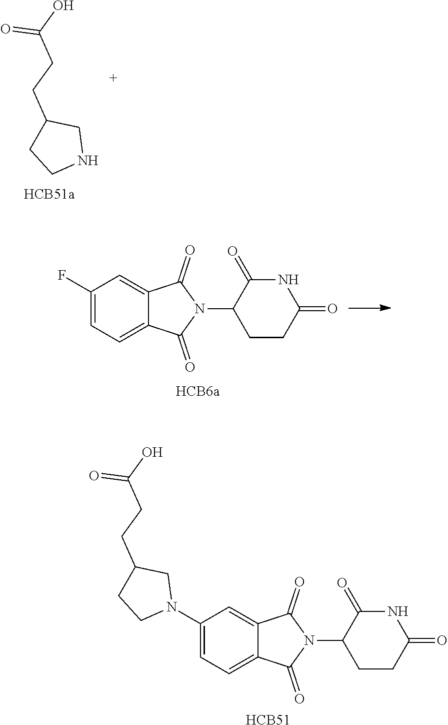

3-(pyrrolidin-3-yl)propanoic acid (100 mg, 0.70 mmol), 2-(2,6-dioxopiperidin-3-yl)-5-fluoroisoindoline-1,3-dione (193 mg, 0.70 mmol), and N,N-diisopropylethylamine (0.49 mL, 2.79 mmol) in DMF (2.00 mL) were heated at 90° C. for three hours. After sitting at room temperature for two days, the mixture was concentrated and then purified by flash chromatography on a 40 g column eluted with zero to 10% MeOH:DCM to provide 3-(1-(2-(2,6-dioxopiperidin-3-yl)-1,3-dioxoisoindolin-5-yl)pyrrolidin-3-yl)propanoic acid (0.0852 g, 30.5%). LCMS: C₂₀H₂₁N₃O₆ requires: 399, found: m/z=400 [M+H]⁺.

### Step 2: 2-(6,6-dimethyl-1-(tetrahydro-2H-pyran-2-yl)-4,5,6,7-tetrahydro-1H-indazol-3-yl)-N-methyl-1H-indol-6-amine (BBXs)

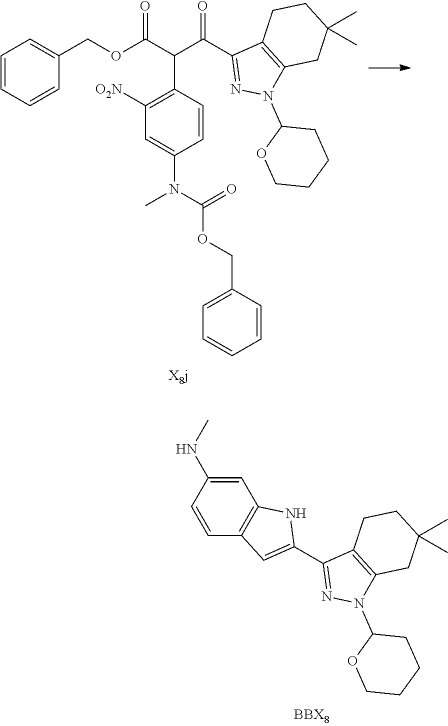 [SMILES: CN(C(=O)OCc1ccccc1)c1ccc(C(C(=O)OCc2ccccc2)C(=O)c2nn(C3CCCCO3)c3c2CCC(C)(C)C3)c([N+](=O)[O-])c1 ; CNc1ccc2cc(-c3nn(C4CCCCO4)c4c3CCC(C)(C)C4)[nH]c2c1]

Benzyl 2-(4-{[(benzyloxy)carbonyl](methyl)amino}-2-nitrophenyl)-3-[6,6-dimethyl-1-(oxan-2-yl)-5,7-dihydro-4H-indazol-3-yl]-3-oxopropanoate (112 mg, 0.16 mmol) (see US 2017/0253577 A1, the contents of which are incorporated herein by reference in their entirety) and 10% Pd/C (11 mg) in toluene (1.00 mL) and EtOH (1.00 mL) were stirred under a balloon of H₂ overnight. The mixture was diluted with THE and then filtered through celite. The mixture was concentrated until about one milliliter of solvent remained, when toluene (3 mL) was then added. The mixture was concentrated down to about one milliliter and the resulting solution of 2-(6,6-dimethyl-1-(tetrahydro-2H-pyran-2-yl)-4,5,6,7-tetrahydro-1H-indazol-3-yl)-N-methyl-1H-indol-6-amine was used in the next step without purification. LCMS: C₂₃H₃₀N₄₀ requires: 378, found: m/z=379 [M+H]⁺.

### Step 3: N-(2-(6,6-dimethyl-1-(tetrahydro-2H-pyran-2-yl)-4,5,6,7-tetrahydro-1H-indazol-3-yl)-1H-indol-6-yl)-3-(1-(2-(2,6-dioxopiperidin-3-yl)-1,3-dioxoisoindolin-5-yl)pyrrolidin-3-yl)-N-methylpropanamide (1a)

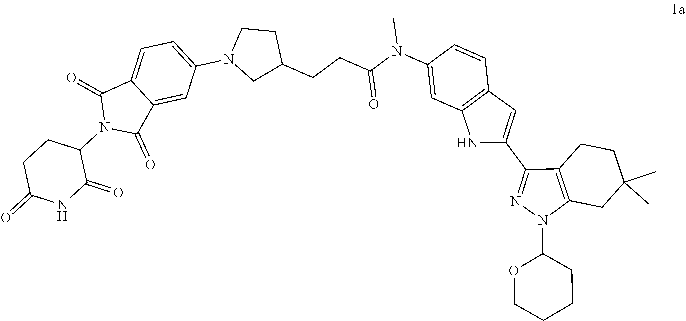 [SMILES: CN(C(=O)CCC1CCN(c2ccc3c(c2)C(=O)N(C2CCC(=O)NC2=O)C3=O)C1)c1ccc2cc(-c3nn(C4CCCCO4)c4c3CCC(C)(C)C4)[nH]c2c1]

To a mixture of 3-(1-(2-(2,6-dioxopiperidin-3-yl)-1,3-dioxoisoindolin-5-yl)pyrrolidin-3-yl)propanoic acid (64 mg, 0.16 mmol) and HATU (61 mg, 0.16 mmol) in DMF (2.00 mL) was added N,N-diisopropylethylamine (0.11 mL, 0.64 mmol). After five minutes, the mixture was added to a solution of 2-(6,6-dimethyl-1-(tetrahydro-2H-pyran-2-yl)-4,5,6,7-tetrahydro-1H-indazol-3-yl)-N-methyl-1H-indol-6-amine (60 mg, 0.16 mmol) in toluene (1 mL). After one hour, the mixture was transferred to a separatory funnel with ethyl acetate. The mixture was washed with water and brine was added to get the layers to separate. The mixture was washed with brine, dried over Na₂SO₄, filtered, and concentrated. The crude residue was purified by flash chromatography on a 24 g column (gradient elution with zero to 10% MeOH:DCM) to provide N-(2-(6,6-dimethyl-1-(tetrahydro-2H-pyran-2-yl)-4,5,6,7-tetrahydro-1H-indazol-3-yl)-1H-indol-6-yl)-3-(1-(2-(2,6-dioxopiperidin-3-yl)-1,3-dioxoisoindolin-5-yl)pyrrolidin-3-yl)-N-methylpropanamide (0.121 g, 100%). LCMS: C₄₃H₄₉N₇O₆ requires: 759, found: m/z=760 [M+H]⁺.

### Step 4: N-(2-(6,6-dimethyl-4,5,6,7-tetrahydro-1H-indazol-3-yl)-1H-indol-6-yl)-3-(1-(2-(2,6-dioxopiperidin-3-yl)-1,3-dioxoisoindolin-5-yl)pyrrolidin-3-yl)-N-methylpropanamide

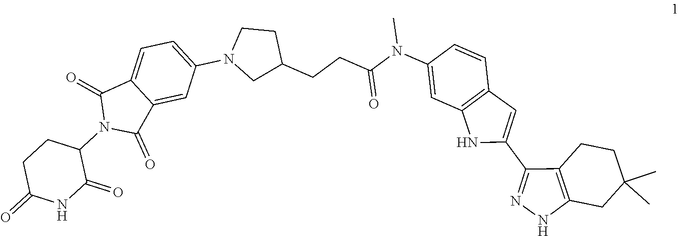 [SMILES: CN(C(=O)CCC1CCN(c2ccc3c(c2)C(=O)N(C2CCC(=O)NC2=O)C3=O)C1)c1ccc2cc(-c3n[nH]c4c3CCC(C)(C)C4)[nH]c2c1]

To a mixture of N-(2-(6,6-dimethyl-1-(tetrahydro-2H-pyran-2-yl)-4,5,6,7-tetrahydro-1H-indazol-3-yl)-1H-indol-6-yl)-3-(1-(2-(2,6-dioxopiperidin-3-yl)-1,3-dioxoisoindolin-5-yl)pyrrolidin-3-yl)-N-methylpropanamide (121 mg, 0.16 mmol) in dioxane (1.00 mL) and EtOH (1.00 mL) was added 4 M hydrogen chloride in dioxane (0.13 mL, 0.53 mmol). The mixture was heated at 50° C. for thirty minutes. The mixture was concentrated then purified by HPLC (5 to 95% MeCN in water with 0.1% TFA) to provide N-(2-(6,6-dimethyl-4,5,6,7-tetrahydro-1H-indazol-3-yl)-1H-indol-6-yl)-3-(1-(2-(2,6-dioxopiperidin-3-yl)-1,3-dioxoisoindolin-5-yl)pyrrolidin-3-yl)-N-methylpropanamide (0.0169 g, 15.6%). ¹H NMR (500 MHz, DMSO-d₆) δ 11.40 (s, 1H), 11.07 (s, 1H), 7.57 (dd, J=11.5, 8.1 Hz, 2H), 7.29 (s, 1H), 6.92 (d, J=8.2 Hz, 1H), 6.82 (s, 1H), 6.69-6.62 (m, 2H), 5.05 (dd, J=12.7, 5.4 Hz, 1H), 3.41 (t, J=8.7 Hz, 2H), 3.33-3.24 (m, 1H), 3.21 (s, 3H), 2.95-2.78 (m, 2H), 2.69 (t, J=6.5 Hz, 2H), 2.63-2.35 (m, 4H), 2.19-2.11 (m, 3H), 2.05-1.95 (m, 2H), 1.72-1.56 (m, 4H), 1.55-1.42 (m, 1H), 1.02 (s, 6H). LCMS: C₃₈H₄₁N₇O₅ requires: 675, found: m/z=676 [M+H]⁺.

### Example 144. N-(2-(6,6-dimethyl-4,5,6,7-tetrahydro-1H-indazol-3-yl)-1H-indol-6-yl)-3-(4-(2-(2,6-dioxopiperidin-3-yl)-1,3-dioxoisoindolin-5-yl)piperazin-1-yl)-N-methylpropanamide (2)

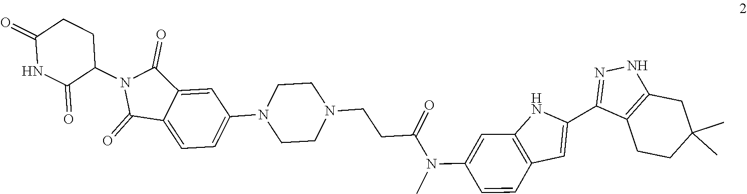 [SMILES: CN(C(=O)CCN1CCN(c2ccc3c(c2)C(=O)N(C2CCC(=O)NC2=O)C3=O)CC1)c1ccc2cc(-c3n[nH]c4c3CCC(C)(C)C4)[nH]c2c1]

N-(2-(6,6-dimethyl-4,5,6,7-tetrahydro-1H-indazol-3-yl)-1H-indol-6-yl)-3-(4-(2-(2,6-dioxopiperidin-3-yl)-1,3-dioxoisoindolin-5-yl)piperazin-1-yl)-N-methylpropanamide (8.9 mg, 25%) was prepared by procedures analogous to Example 79 starting from 2-[6,6-dimethyl-1-(oxan-2-yl)-5,7-dihydro-4H-indazol-3-yl]-N-methyl-1H-indol-6-amine (19.00 mg, 0.05 mmol) and 3-{4-[2-(2,6-dioxopiperidin-3-yl)-1,3-dioxoisoindol-5-yl]piperazin-1-yl}propanoic acid (20.80 mg, 0.05 mmol). ¹H NMR (500 MHz, DMSO-d₆) δ 11.50 (s, 1H), 11.10 (s, 1H), 9.28 (s, 1H), 7.75 (d, J=8.4 Hz, 1H), 7.62 (d, J=8.2 Hz, 1H), 7.45 (s, 1H), 7.36-7.29 (m, 2H), 6.96 (d, J=8.2 Hz, 1H), 6.65 (s, 1H), 5.09 (dd, J=12.8, 5.4 Hz, 1H), 4.16 (d, J=12.5 Hz, 2H), 3.26 (s, 3H), 3.22-3.09 (m, 4H), 2.89 (ddd, J=17.9, 13.8, 5.4 Hz, 1H), 2.73-2.34 (m, 12H), 2.06-2.00 (m, 1H), 1.59 (t, J=6.4 Hz, 2H), 1.02 (s, 6H). LCMS: C₃₈H₄₂N₈O₅ requires: 690, found: m/z=691 [M+H]⁺.

### Intermediate BBX₉

### Example 145. (S)—N-(2-(6,6-dimethyl-4,5,6,7-tetrahydro-1H-indazol-3-yl)-1H-indol-6-yl)-N-methyl-2-(piperazin-1-yl)propanamide (BBX₉)

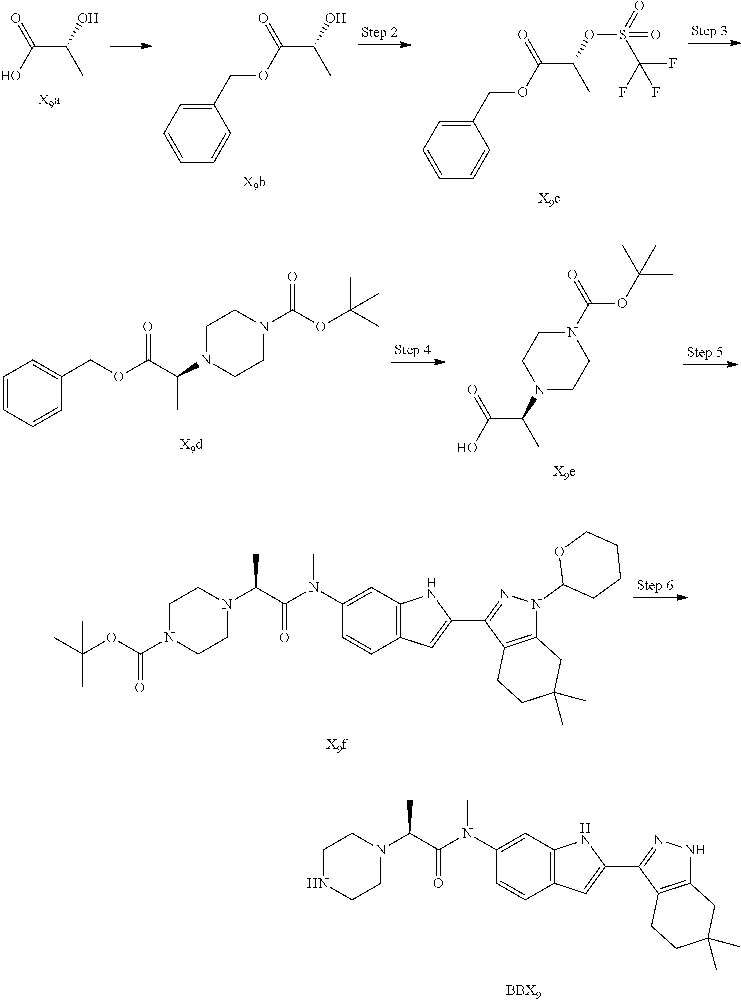

### Step 1: benzyl (2R)-2-hydroxypropanoate (X₉b)

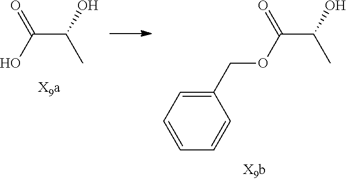

D-lactic acid (4.06 g, 45.07 mmol) was dissolved in MeOH (40 mL). A 20% solution of cesium carbonate (7.34 g, 22.5 mmol) in water was added until the solution reached pH 7. The mixture was then concentrated and the resulting material was dissolved in toluene and concentrated in vacuo twice. The resulting material was dissolved in DMF (100 mL) and benzyl bromide (6.43 mL, 54.1 mmol) was added. After stirring overnight, the mixture was concentrated. Ethyl acetate (100 mL) was added and the mixture was filtered. The resulting solution was washed with water, brine, and concentrated in vacuo. The crude residue was purified by flash chromatography on an 80 g column eluted with zero to 30% ethyl acetate:DCM then was repurified by flash chromatography on an 80 g column eluted with zero to 35% ethyl acetate:hexanes to provide benzyl (2R)-2-hydroxypropanoate (4.360 g, 53.7%). LCMS: C₁₀H1203 requires: 180, found: m/z=203 [M+Na]⁺.

### Step 2: benzyl (2R)-2-(trifluoromethanesulfonyloxy)propanoate (X₉c)

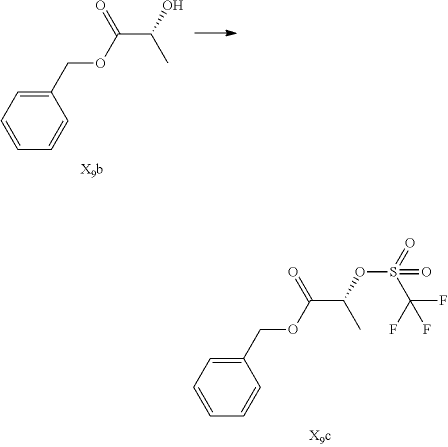 [SMILES: C[C@@H](O)C(=O)OCc1ccccc1 ; C[C@@H](OS(=O)(=O)C(F)(F)F)C(=O)OCc1ccccc1]

To a 0° C. mixture of benzyl (2R)-2-hydroxypropanoate (2.79 g, 15.5 mmol) and lutidine (1.90 mL, 16.3 mmol) in DCM (75 mL) was added triflic anhydride (2.67 mL, 16.3 mmol). After one hour, the mixture was concentrated in vacuo. The crude material was purified by flash chromatography on a 120 g column eluted with zero to 100% ethyl acetate:hexanes to provide benzyl (2R)-2-(trifluoromethanesulfonyloxy)propanoate (3.94 g, 81.3%). ¹H NMR (500 MHz, CDCl₃) δ 7.45-7.35 (m, 5H), 5.34-5.25 (m, 3H), 1.74 (d, J=7.0 Hz, 3H).

### Step 3: tert-butyl 4-[(2S)-1-(benzyloxy)-1-oxopropan-2-yl]piperazine-1-carboxylate (X₉d)

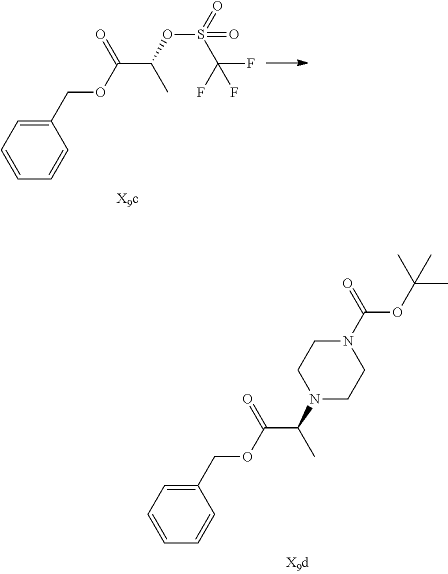 [SMILES: C[C@@H](OS(=O)(=O)C(F)(F)F)C(=O)OCc1ccccc1 ; C[C@@H](C(=O)OCc1ccccc1)N1CCN(C(=O)OC(C)(C)C)CC1]

A mixture of benzyl (2R)-2-(trifluoromethanesulfonyloxy)propanoate (3.94 g, 12.6 mmol) in MeCN (30 mL) was added to a mixture of tert-butyl piperazine-1-carboxylate (2.35 g, 12.6 mmol) and potassium carbonate (3.49 g, 25.2 mmol) in MeCN (30 mL). The resulting mixture was heated at 60° C. for one hour. The resulting mixture was filtered and the solids were washed with DCM. The resulting solution was concentrated onto silica gel and purified by flash chromatography on an 80 g column eluted with zero to 50% ethyl acetate:hexanes to provide tert-butyl 4-[(2S)-1-(benzyloxy)-1-oxopropan-2-yl]piperazine-1-carboxylate (4.100 g, 93.3%). LCMS: C₁₉H₂₈N₂O₄ requires: 348, found: m/z=349 [M+H]⁺.

### Step 4: (2S)-2-[4-(tert-butoxycarbonyl)piperazin-1-yl]propanoic acid (X₉e)

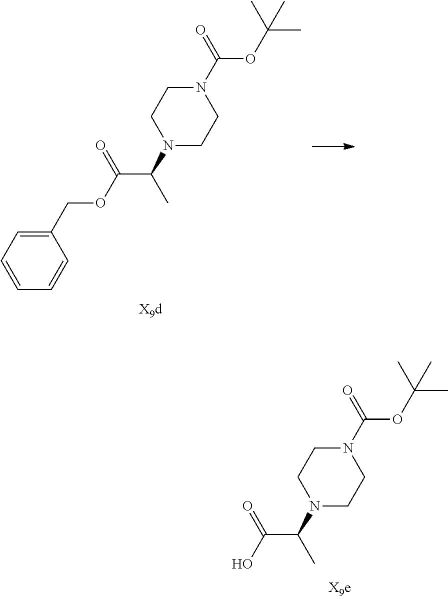 [SMILES: C[C@@H](C(=O)OCc1ccccc1)N1CCN(C(=O)OC(C)(C)C)CC1 ; C[C@@H](C(=O)O)N1CCN(C(=O)OC(C)(C)C)CC1]

A mixture of tert-butyl 4-[(2S)-1-(benzyloxy)-1-oxopropan-2-yl]piperazine-1-carboxylate (4.10 g, 11.8 mmol) and 10% Pd/C (410 mg) was stirred in EtOH (50 mL) under a balloon of H₂ for ninety minutes. The mixture was filtered through celite which was washed with MeOH and DCM. The combined solutions were concentrated to provide (2S)-2-[4-(tert-butoxycarbonyl)piperazin-1-yl]propanoic acid (2.876 g, 94.6%). LCMS: C₁₂H₂₂N₂O₄ requires: 258, found: m/z=259 [M+H]⁺.

### Step 5: tert-butyl 4-[(1S)-1-({2-[6,6-dimethyl-1-(oxan-2-yl)-5,7-dihydro-4H-indazol-3-yl]-1H-indol-6-yl}(methyl)carbamoyl)ethyl]piperazine-1-carboxylate (X₉f)

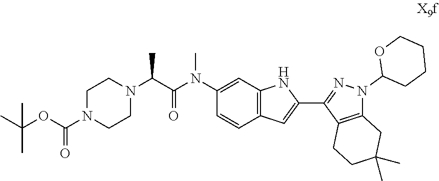 [SMILES: C[C@@H](C(=O)N(C)c1ccc2cc(-c3nn(C4CCCCO4)c4c3CCC(C)(C)C4)[nH]c2c1)N1CCN(C(=O)OC(C)(C)C)CC1]

N,N-diisopropylethylamine (0.45 mL, 2.60 mmol) was added to a mixture of (2S)-2-[4-(tert-butoxycarbonyl)piperazin-1-yl]propanoic acid (168 mg, 0.65 mmol) and HATU (247 mg, 0.65 mmol) in DMF (3.00 mL). After five minutes, the resulting mixture was added to a solution of 2-[6,6-dimethyl-1-(oxan-2-yl)-5,7-dihydro-4H-indazol-3-yl]-N-methyl-1H-indol-6-amine (246.17 mg, 0.65 mmol) in toluene (1 mL). After ninety minutes, water was added and the mixture was extracted with ethyl acetate. Brine was added to get the layers to separate and the organic layer was dried over Na₂SO₄, filtered, and concentrated in vacuo. The crude residue was purified by flash chromatography on a 24 g column eluted with zero to 5% MeOH:DCM to provide tert-butyl 4-[(1S)-1-({2-[6,6-dimethyl-1-(oxan-2-yl)-5,7-dihydro-4H-indazol-3-yl]-1H-indol-6-yl}(methyl)carbamoyl)ethyl]piperazine-1-carboxylate (0.401 g, 100%). LCMS: C₃₅H₅₀N₆O₄ requires: 618, found: m/z=619 [M+H]⁺.

### Step 6: (S)—N-(2-(6,6-dimethyl-4,5,6,7-tetrahydro-1H-indazol-3-yl)-1H-indol-6-yl)-N-methyl-2-(piperazin-1-yl)propanamide (BBX₉)

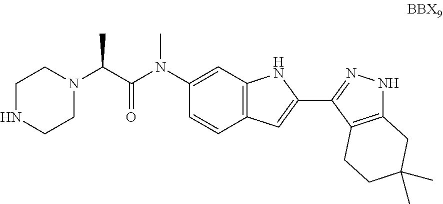 [SMILES: C[C@@H](C(=O)N(C)c1ccc2cc(-c3n[nH]c4c3CCC(C)(C)C4)[nH]c2c1)N1CCNCC1]

tert-butyl 4-[(1S)-1-({2-[6,6-dimethyl-1-(oxan-2-yl)-5,7-dihydro-4H-indazol-3-yl]-1H-indol-6-yl}(methyl)carbamoyl)ethyl]piperazine-1-carboxylate (401 mg, 0.65 mmol) was stirred in EtOH (2.00 mL) and 4 M HCl in dioxane (2.00 mL, mg, 8.00 mmol) for fifteen minutes. The mixture was concentrated in vacuo to provide (S)—N-(2-(6,6-dimethyl-4,5,6,7-tetrahydro-1H-indazol-3-yl)-1H-indol-6-yl)-N-methyl-2-(piperazin-1-yl)propanamide (0.281 g, 100%) as an HCl salt. LCMS: C₂₅H₃₄N₆O requires: 434, found: m/z=435 [M+H]⁺.

### Example 146. (2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-4-yl)piperidin-4-yl)methyl)piperazin-1-yl)-N-methylpropanamide (3)

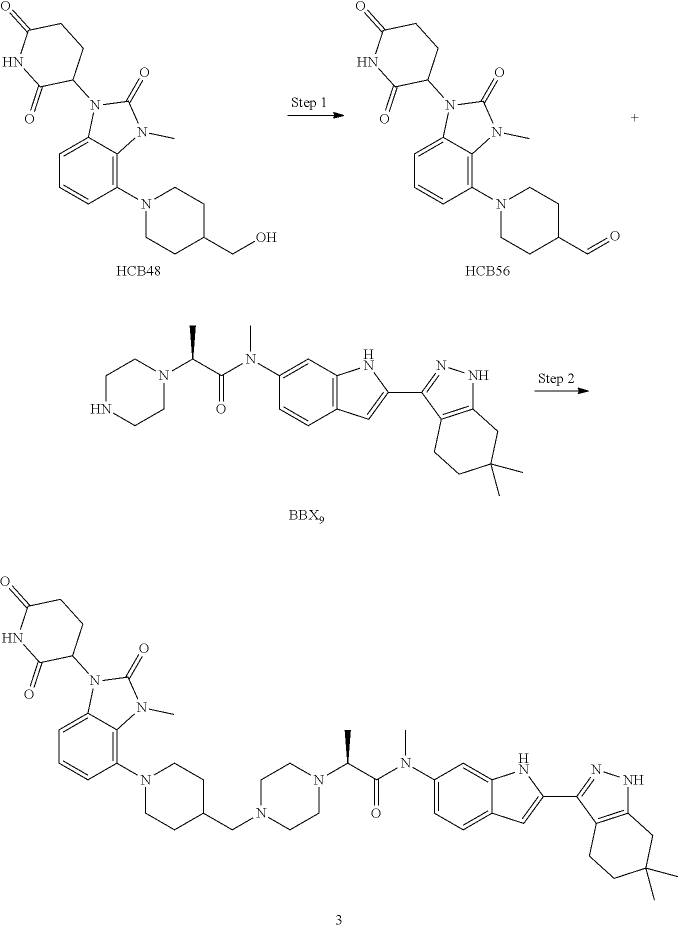

### Step 1: 1-(1-(2,6-dioxopiperidin-3-yl)-3-methyl-2-oxo-2,3-dihydro-1H-benzo[d]imidazol-4-yl)piperidine-4-carbaldehyde (HCB56)

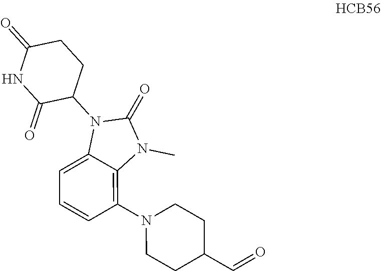 [SMILES: Cn1c(=O)n(C2CCC(=O)NC2=O)c2cccc(N3CCC(C=O)CC3)c21]

To a mixture of 3-(4-(4-(hydroxymethyl)piperidin-1-yl)-3-methyl-2-oxo-2,3-dihydro-1H-benzo[d]imidazol-1-yl)piperidine-2,6-dione (33 mg, 0.09 mmol) and triethylamine (0.25 mL, 1.8 mmol) in DMSO (1.00 mL) was added sulfur trioxide pyridine complex (141 mg, 0.89 mmol). After twenty minutes, the starting material was consumed as seen by TLC in 10% MeOH:DCM. Water was added and the mixture was extracted twice with DCM. The combined organic layers were concentrated to provide a mixture containing 1-(1-(2,6-dioxopiperidin-3-yl)-3-methyl-2-oxo-2,3-dihydro-1H-benzo[d]imidazol-4-yl)piperidine-4-carbaldehyde and DMSO. LCMS: C₁₉H₂₂N₄O₄ requires: 371, found: m/z=371 [M+H]⁺.

### Step 2: (2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-4-yl)piperidin-4-yl)methyl)piperazin-1-yl)-N-methylpropanamide (3)

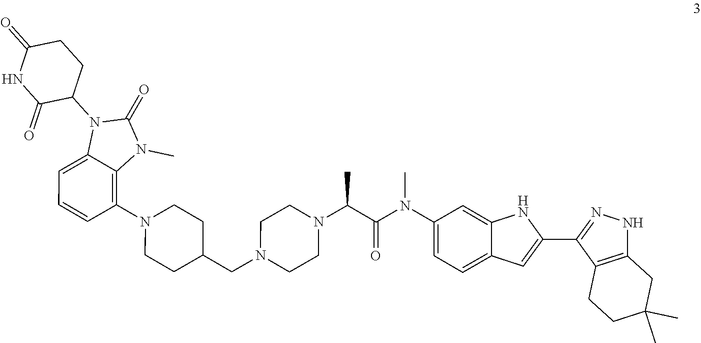 [SMILES: C[C@@H](C(=O)N(C)c1ccc2cc(-c3n[nH]c4c3CCC(C)(C)C4)[nH]c2c1)N1CCN(CC2CCN(c3cccc4c3n(C)c(=O)n4C3CCC(=O)NC3=O)CC2)CC1]

To a mixture of (2S)—N-[2-(6,6-dimethyl-1,4,5,7-tetrahydroindazol-3-yl)-1H-indol-6-yl]-N-methyl-2-(piperazin-1-yl)propanamide (38.7 mg, 0.09 mmol) in DCM (1.00 mL) was added N,N-diisopropylethylamine (0.06 mL, 0.36 mmol). The resulting mixture was added to 1-(1-(2,6-dioxopiperidin-3-yl)-3-methyl-2-oxo-2,3-dihydro-1H-benzo[d]imidazol-4-yl)piperidine-4-carbaldehyde (0.09 mmol) followed by sodium triacetoxyborohydride (38 mg, 0.18 mmol). After thirty minutes, water was added and the mixture was extracted twice with DCM. The combined organic layers were concentrated then purified by prep-HPLC (10 to 95% MeCN in water with 0.1% TFA) to provide (2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-4-yl)piperidin-4-yl)methyl)piperazin-1-yl)-N-methylpropanamide (0.0284 g, 38.8%). ¹H NMR (500 MHz, DMSO-d₆) δ 11.43 (s, 1H), 11.10 (s, 1H), 9.00 (s, 1H), 7.60 (d, J=8.3 Hz, 1H), 7.32 (s, 1H), 7.03-6.87 (m, 4H), 6.65 (s, 1H), 5.36 (dd, J=13.0, 5.4 Hz, 1H), 3.63 (s, 3H), 3.57-3.32 (m, 2H), 3.20-2.81 (m, 8H), 2.80-2.58 (m, 7H), 2.59-2.29 (m, 7H), 2.03-1.97 (m, 1H), 1.86-1.80 (m, 3H), 1.62-1.56 (m, 2H), 1.52-1.32 (m, 2H), 1.30-1.07 (m, 4H), 1.02 (s, 6H). LCMS: C₄₄H₅₆N₁₀O₄ requires: 788, found: m/z=789 [M+H]⁺.

### Example 147. (2S)—N-(2-(6,6-dimethyl-4,5,6,7-tetrahydro-1H-indazol-3-yl)-1H-indol-6-yl)-2-(4-(((3S)-1-(1-(2,6-dioxopiperidin-3-yl)-3-methyl-2-oxo-2,3-dihydro-1H-benzo[d]imidazol-5-yl)pyrrolidin-3-yl)methyl)piperazin-1-yl)-N-methylpropanamide (4)

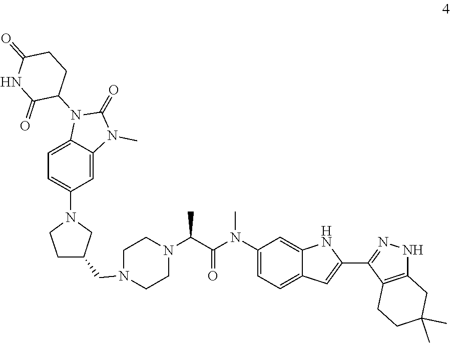 [SMILES: C[C@@H](C(=O)N(C)c1ccc2cc(-c3n[nH]c4c3CCC(C)(C)C4)[nH]c2c1)N1CCN(C[C@@H]2CCN(c3ccc4c(c3)n(C)c(=O)n4C3CCC(=O)NC3=O)C2)CC1]

(2S)—N-(2-(6,6-dimethyl-4,5,6,7-tetrahydro-1H-indazol-3-yl)-1H-indol-6-yl)-2-(4-(((3S)-1-(1-(2,6-dioxopiperidin-3-yl)-3-methyl-2-oxo-2,3-dihydro-1H-benzo[d]imidazol-5-yl)pyrrolidin-3-yl)methyl)piperazin-1-yl)-N-methylpropanamide (0.0287 g, 38.2%) was prepared by procedures analogous to Example 82 starting from 3-(5-((R)-3-(hydroxymethyl)pyrrolidin-1-yl)-3-methyl-2-oxo-2,3-dihydro-1H-benzo[d]imidazol-1-yl)piperidine-2,6-dione (33.5 mg, 0.09 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (40.4 mg, 0.09 mmol). ¹H NMR (500 MHz, DMSO-d₆) δ 11.44 (s, 1H), 11.06 (s, 1H), 9.25 (s, 1H), 7.61 (d, J=8.2 Hz, 1H), 7.33 (s, 1H), 6.98-6.90 (m, 2H), 6.65 (d, J=2.0 Hz, 1H), 6.38 (d, J=2.1 Hz, 1H), 6.24 (d, J=8.7 Hz, 1H), 5.28 (dd, J=12.9, 5.3 Hz, 1H), 3.51-3.39 (m, 2H), 3.36-3.21 (m, 8H), 3.05-2.87 (m, 4H), 2.77-2.59 (m, 4H), 2.57-2.36 (m, 11H), 2.20-2.16 (m, 1H), 2.02-1.96 (m, 1H), 1.76-1.72 (m, 1H), 1.59 (t, J=6.5 Hz, 2H), 1.27-1.09 (m, 3H), 1.02 (s, 6H). LCMS: C₄₃H₅₄N₁₀O₄ requires: 774, found: m/z=775 [M+H]⁺.

### Example 148. (2S)—N-(2-(6,6-dimethyl-4,5,6,7-tetrahydro-1H-indazol-3-yl)-1H-indol-6-yl)-2-(4-(((3R)-1-(1-(2,6-dioxopiperidin-3-yl)-3-methyl-2-oxo-2,3-dihydro-1H-benzo[d]imidazol-5-yl)pyrrolidin-3-yl)methyl)piperazin-1-yl)-N-methylpropanamide (5)

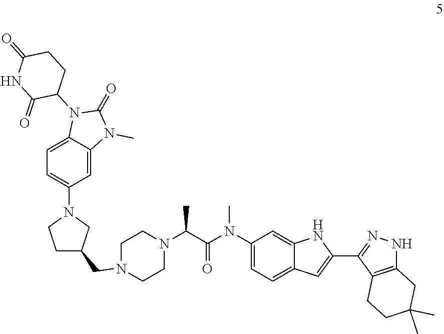 [SMILES: C[C@@H](C(=O)N(C)c1ccc2cc(-c3n[nH]c4c3CCC(C)(C)C4)[nH]c2c1)N1CCN(C[C@H]2CCN(c3ccc4c(c3)n(C)c(=O)n4C3CCC(=O)NC3=O)C2)CC1]

(2S)—N-(2-(6,6-dimethyl-4,5,6,7-tetrahydro-1H-indazol-3-yl)-1H-indol-6-yl)-2-(4-(((3R)-1-(1-(2,6-dioxopiperidin-3-yl)-3-methyl-2-oxo-2,3-dihydro-1H-benzo[d]imidazol-5-yl)pyrrolidin-3-yl)methyl)piperazin-1-yl)-N-methylpropanamide (0.0371 g, 48.9%) was prepared by procedures analogous to Example 82 starting from 3-(5-((S)-3-(hydroxymethyl)pyrrolidin-1-yl)-3-methyl-2-oxo-2,3-dihydro-1H-benzo[d]imidazol-1-yl)piperidine-2,6-dione (33.4 mg, 0.09 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (40.42 mg, 0.09 mmol). ¹H NMR (500 MHz, DMSO-d₆) δ 11.44 (s, 1H), 11.06 (s, 1H), 9.21 (s, 1H), 7.61 (d, J=8.2 Hz, 1H), 7.33 (s, 1H), 6.98-6.90 (m, 2H), 6.65 (d, J=2.0 Hz, 1H), 6.39 (d, J=2.2 Hz, 1H), 6.24 (d, J=8.4 Hz, 1H), 5.28 (dd, J=12.9, 5.3 Hz, 1H), 3.66-3.38 (m, 3H), 3.37-3.28 (m, 4H), 3.28-3.20 (m, 4H), 3.06-2.83 (m, 5H), 2.80-2.60 (m, 7H), 2.59-2.33 (m, 6H), 2.20-2.16 (m, 1H), 2.01-1.96 (m, 1H), 1.76-1.73 (m, 1H), 1.59 (t, J=6.4 Hz, 2H), 1.31-1.08 (m, 3H), 1.02 (s, 6H). LCMS: C₄₃H₅₄N₁₀O₄ requires: 774, found: m/z=775 [M+H]⁺.

### Example 149. (2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-methylpropanamide (6)

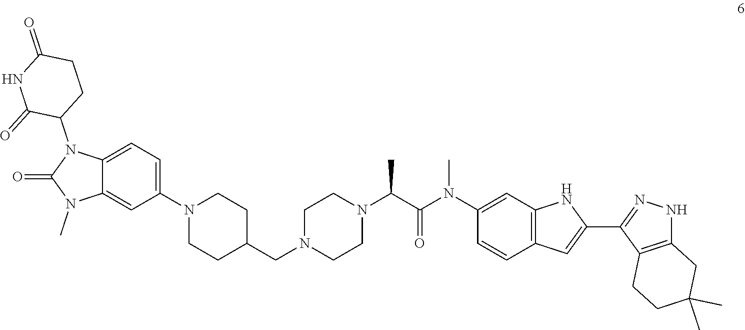 [SMILES: C[C@@H](C(=O)N(C)c1ccc2cc(-c3n[nH]c4c3CCC(C)(C)C4)[nH]c2c1)N1CCN(CC2CCN(c3ccc4c(c3)n(C)c(=O)n4C3CCC(=O)NC3=O)CC2)CC1]

(2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-methylpropanamide (2S)—N-(2-(6,6-dimethyl-4,5,6,7-tetrahydro-1H-indazol-3-yl)-1H-indol-6-yl)-2-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-methylpropanamide (0.0399 g, 43.5%) was prepared by procedures analogous to Example 82 starting from 3-(5-(4-(hydroxymethyl)piperidin-1-yl)-3-methyl-2-oxo-2,3-dihydro-1H-benzo[d]imidazol-1-yl)piperidine-2,6-dione (48.0 mg, 0.13 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (50.0 mg, 0.12 mmol). ¹H NMR (500 MHz, DMSO-d₆) δ 11.44 (s, 1H), 11.10 (s, 1H), 9.12 (s, 1H), 7.61 (d, J=8.4 Hz, 1H), 7.33 (s, 1H), 7.19-7.00 (m, 3H), 6.96 (d, J=8.5 Hz, 1H), 6.65 (s, 1H), 5.35 (d, J=13.2 Hz, 1H), 3.63 (d, J=11.3 Hz, 2H), 3.55-3.31 (m, 4H), 3.30-3.19 (m, 3H), 3.14-2.59 (m, 10H), 2.59-2.34 (m, 9H), 2.04-1.98 (m, 1H), 1.90-1.87 (m, 3H), 1.59 (t, J=6.4 Hz, 2H), 1.42 (s, 2H), 1.30-1.08 (m, 3H), 1.02 (s, 6H). LCMS: C₄₄H₅₆N₁₀O₄ requires: 788, found: m/z=789 [M+H]⁺.

### Example 150. 5-(4-((4-((S)-1-((2-(6,6-dimethyl-4,5,6,7-tetrahydro-1H-indazol-3-yl)-1H-indol-6-yl)(methyl)amino)-1-oxopropan-2-yl)piperazin-1-yl)methyl)piperidin-1-yl)-N-(2,6-dioxopiperidin-3-yl)picolinamide (7)

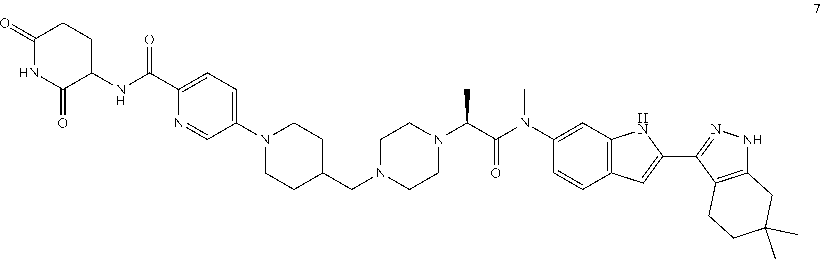 [SMILES: C[C@@H](C(=O)N(C)c1ccc2cc(-c3n[nH]c4c3CCC(C)(C)C4)[nH]c2c1)N1CCN(CC2CCN(c3ccc(C(=O)NC4CCC(=O)NC4=O)nc3)CC2)CC1]

5-(4-((4-((S)-1-((2-(6,6-dimethyl-4,5,6,7-tetrahydro-1H-indazol-3-yl)-1H-indol-6-yl)(methyl)amino)-1-oxopropan-2-yl)piperazin-1-yl)methyl)piperidin-1-yl)-N-(2,6-dioxopiperidin-3-yl)picolinamide (0.0336 g, 41.2%) was prepared by procedures analogous to Example 82 starting from N-(2,6-dioxopiperidin-3-yl)-5-(4-formylpiperidin-1-yl)picolinamide (36.45 mg, 0.11 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (46.00 mg, 0.11 mmol). ¹H NMR (500 MHz, DMSO-d₆) δ 11.44 (s, 1H), 10.86 (s, 1H), 9.04 (s, 1H), 8.70 (dd, J=8.3, 2.4 Hz, 1H), 8.33 (d, J=2.9 Hz, 1H), 7.87 (dd, J=8.9, 2.4 Hz, 1H), 7.61 (d, J=8.2 Hz, 1H), 7.43 (dd, J=8.8, 2.9 Hz, 1H), 7.33 (s, 1H), 6.95 (d, J=8.3 Hz, 1H), 6.65 (d, J=2.3 Hz, 1H), 4.75 (ddd, J=13.1, 8.3, 5.3 Hz, 1H), 3.97 (d, J=12.7 Hz, 2H), 3.57-3.34 (m, 2H), 3.25 (s, 3H), 3.10-2.74 (m, 8H), 2.69 (t, J=6.4 Hz, 2H), 2.59-2.53 (m, 2H), 2.43 (s, 2H), 2.24-2.12 (m, 1H), 2.10-1.98 (m, 3H), 1.80 (d, J=12.7 Hz, 2H), 1.59 (t, J=6.3 Hz, 2H), 1.33-1.10 (m, 7H), 1.03 (s, 6H). LCMS: C₄₂H₅₄N₁₀O₄ requires: 762, found: m/z=763 [M+H]⁺.

### Intermediate BBX₁₀

### 2-[2-(6,6-dimethyl-1,4,5,7-tetrahydroindazol-3-yl)-1H-indol-6-yl]-2,8-diazaspiro[4.5]decan-1-one (BBX₁₀)

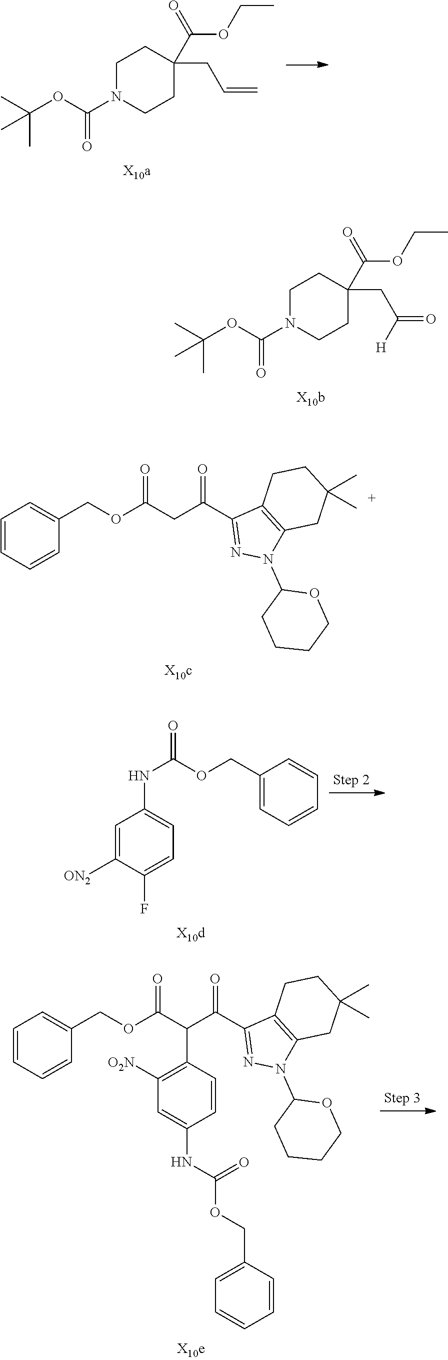

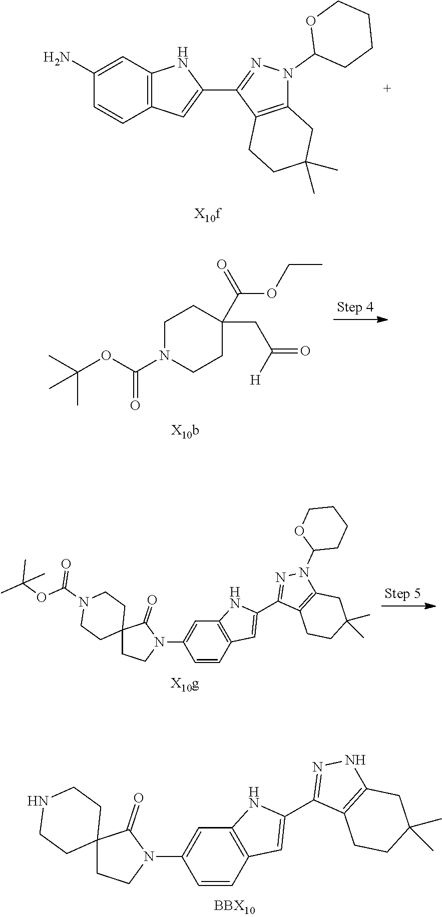

### Step 1: 1-tert-butyl 4-ethyl 4-(2-oxoethyl)piperidine-1,4-dicarboxylate (X_{10b})

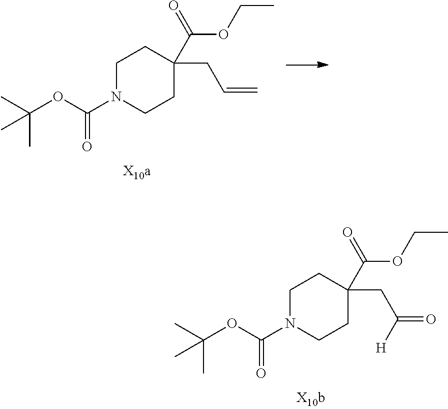

To a mixture of 1-tert-butyl 4-ethyl 4-(prop-2-en-1-yl)piperidine-1,4-dicarboxylate (2.23 g, 7.51 mmol) in THF (11.00 mL) and water (11.00 mL) was added osmium tetroxide (80.3 mg, 0.32 mmol). Five minutes later sodium periodate (4.01 g, 18.77 mmol) was added. After three hours, the mixture was partitioned between ethyl acetate and aq. NaHCO₃. The organic layer was dried over Na₂SO₄, filtered, and concentrated. The crude residue was purified by flash chromatography on a 40 g column with gradient elution zero to 40% ethyl acetate:hexanes to provide (1.7670 g, 78.6%). LCMS: C₁₅H₂₅NO₅ requires: 299, found: m/z=322 [M+Na]⁺.

### Step 2: benzyl 2-(4-{[(benzyloxy)carbonyl]amino}-2-nitrophenyl)-3-[6,6-dimethyl-1-(oxan-2-yl)-5,7-dihydro-4H-indazol-3-yl]-3-oxopropanoate (X₁₀e)

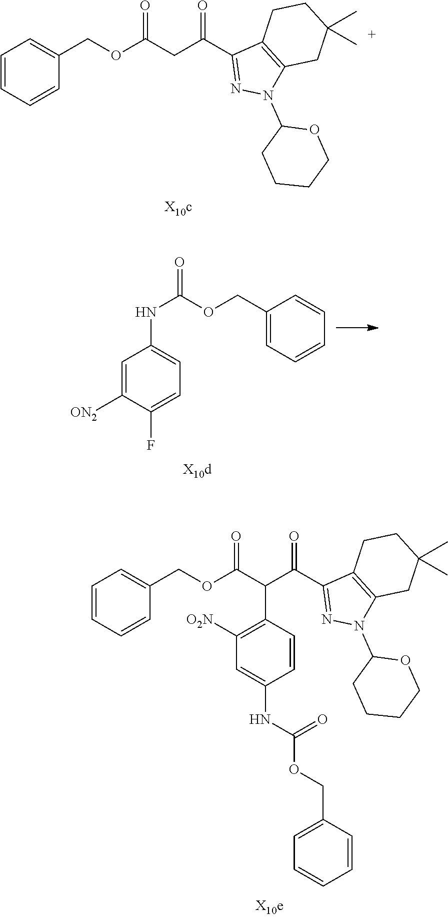

To a mixture of benzyl 3-[6,6-dimethyl-1-(oxan-2-yl)-5,7-dihydro-4H-indazol-3-yl]-3-oxopropanoate (606 mg, 1.48 mmol) in DMSO (4.00 mL) was added benzyl N-(4-fluoro-3-nitrophenyl)carbamate (428 mg, 1.48 mmol) and potassium carbonate (408 mg, 2.95 mmol). The mixture was heated at 90° C. for two hours. The mixture was cooled and partitioned between ethyl acetate and 5% aq. citric acid. The organic layer was washed with brine, dried over Na₂SO₄, filtered, and concentrated in vacuo. The crude residue was purified by flash chromatography on a 40 g column eluted with zero to 40% ethyl acetate:hexanes to provide benzyl 2-(4-{[(benzyloxy)carbonyl]amino}-2-nitrophenyl)-3-[6,6-dimethyl-1-(oxan-2-yl)-5,7-dihydro-4H-indazol-3-yl]-3-oxopropanoate (0.375 g, 37.3%). LCMS: C₃₈H₄₀N₄O₈ requires: 680, found: m/z=681 [M+H]⁺.

### Step 3: of 2-[6,6-dimethyl-1-(oxan-2-yl)-5,7-dihydro-4H-indazol-3-yl]-1H-indol-6-amine (X₁₀f)

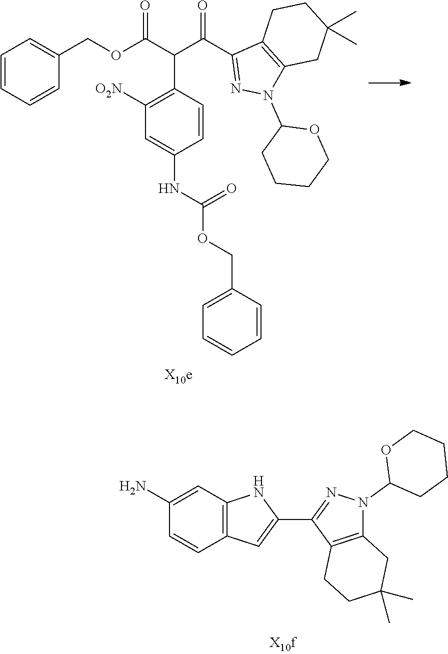 [SMILES: CC1(C)CCc2c(C(=O)C(C(=O)OCc3ccccc3)c3ccc(NC(=O)OCc4ccccc4)cc3[N+](=O)[O-])nn(C3CCCCO3)c2C1 ; CC1(C)CCc2c(-c3cc4ccc(N)cc4[nH]3)nn(C3CCCCO3)c2C1]

To a mixture of benzyl 2-(4-{[(benzyloxy)carbonyl]amino}-2-nitrophenyl)-3-[6,6-dimethyl-1-(oxan-2-yl)-5,7-dihydro-4H-indazol-3-yl]-3-oxopropanoate (210 mg, 0.31 mmol) in EtOH (4.00 mL) and toluene (4.00 mL) was added 10% Pd/C (21 mg). The mixture was stirred under a balloon of H₂ for eighteen hours. The mixture was diluted with THF and filtered through celite. Toluene (5 mL) was added and the mixture was concentrated down to about one milliliter. Toluene (5 mL) was added and the mixture was again concentrated down to about one milliliter and was taken on to the next step immediately as a solution of 2-[6,6-dimethyl-1-(oxan-2-yl)-5,7-dihydro-4H-indazol-3-yl]-1H-indol-6-amine in toluene. LCMS: C₂₂H₂₈N₄₀ requires: 364, found: m/z=365 [M+H]⁺.

### Step 4: tert-butyl 2-{2-[6,6-dimethyl-1-(oxan-2-yl)-5,7-dihydro-4H-indazol-3-yl]-1H-indol-6-yl}-1-oxo-2,8-diazaspiro[4.5]decane-8-carboxylate (X_{10g})

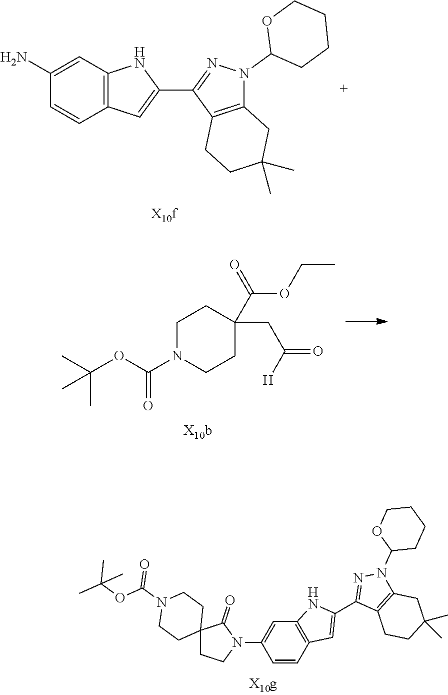

A solution of 2-[6,6-dimethyl-1-(oxan-2-yl)-5,7-dihydro-4H-indazol-3-yl]-1H-indol-6-amine (113 mg, 0.31 mmol) in toluene (1 mL) was added to 1-tert-butyl 4-ethyl 4-(2-oxoethyl)piperidine-1,4-dicarboxylate (92 mg, 0.31 mmol) with THF (2.00 mL) followed by acetic acid (9.3 mg, 0.15 mmol) and sodium triacetoxyborohydride (131 mg, 0.62 mmol). After one hour, the mixture was diluted with toluene (3 mL) and heated at 100° C. for 2.5 hours. The mixture was cooled and diluted with ethyl acetate. The mixture was washed with dilute aqueous sodium bicarbonate solution and the organic layer was concentrated. The crude residue was purified by flash chromatography on a 24 g column eluted with zero to 75% ethyl acetate:DCM to provide tert-butyl 2-{2-[6,6-dimethyl-1-(oxan-2-yl)-5,7-dihydro-4H-indazol-3-yl]-1H-indol-6-yl}-1-oxo-2,8-diazaspiro[4.5]decane-8-carboxylate (0.0690 g, 37.0%). LCMS: C₃₅H₄₇N₅O₄ requires: 601, found: m/z=602 [M+H]⁺.

### Step 5: 2-[2-(6,6-dimethyl-1,4,5,7-tetrahydroindazol-3-yl)-1H-indol-6-yl]-2,8-diazaspiro[4.5]decan-1-one (BBX₁₀)

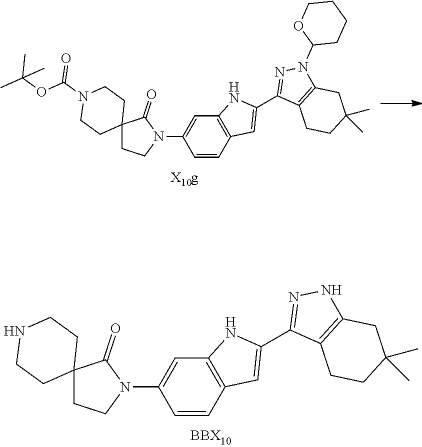 [SMILES: CC1(C)CCc2c(-c3cc4ccc(N5CCC6(CCN(C(=O)OC(C)(C)C)CC6)C5=O)cc4[nH]3)nn(C3CCCCO3)c2C1 ; CC1(C)CCc2c(-c3cc4ccc(N5CCC6(CCNCC6)C5=O)cc4[nH]3)n[nH]c2C1]

tert-butyl 2-{2-[6,6-dimethyl-1-(oxan-2-yl)-5,7-dihydro-4H-indazol-3-yl]-1H-indol-6-yl}-1-oxo-2,8-diazaspiro[4.5]decane-8-carboxylate (69 mg, 0.11 mmol) and para-toluene sulfonate hydrate (44 mg, 0.23 mmol) were dissolved in EtOH (2.00 mL) and heated to 85° C. for 25 minutes. The mixture was cooled, dilute aqueous sodium bicarbonate was added, and the mixture was extracted three times with DCM. The combined organic layers were dried over Na₂SO₄, filtered, and concentrated to provide 2-[2-(6,6-dimethyl-1,4,5,7-tetrahydroindazol-3-yl)-1H-indol-6-yl]-2,8-diazaspiro[4.5]decan-1-one (0.043 g, 90%). LCMS: C₂₅H₃₁N₅₀ requires: 417, found: m/z=418 [M+H]⁺.

### Example 151. tert-butyl 2-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-1-oxo-2,8-diazaspiro[4.5]decane-8-carboxylate (BBX₁₁)

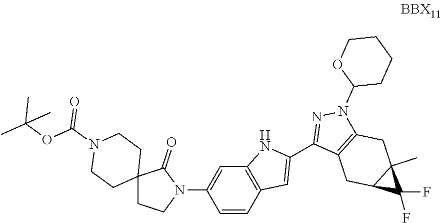 [SMILES: CC(C)(C)OC(=O)N1CCC2(CC1)CCN(c1ccc3cc(-c4nn(C5CCCCO5)c5c4C[C@@H]4C(F)(F)[C@]4(C)C5)[nH]c3c1)C2=O]

tert-butyl 2-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-1-oxo-2,8-diazaspiro[4.5]decane-8-carboxylate was prepared by procedures analogous to Example 86 starting from (4aS,5aR)-5,5-difluoro-5a-methyl-1,4,4a,5,5a,6-hexahydrocyclopropafindazole-3-carboxylic acid. LCMS: C₃₅H₄₃F₂N₅O₄ requires: 635, found: m/z=636 [M+H]⁺.

### Example 152. 2-{2-[(4aS,5aR)-5,5-difluoro-5a-methyl-1H,4H,4aH,6H-cyclopropa[f]indazol-3-yl]-1H-indol-6-yl}-2,8-diazaspiro[4.5]decan-1-one (BBX₁₃)

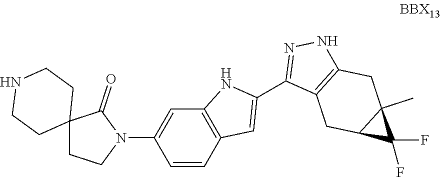 [SMILES: C[C@@]12Cc3[nH]nc(-c4cc5ccc(N6CCC7(CCNCC7)C6=O)cc5[nH]4)c3C[C@@H]1C2(F)F]

To a mixture of tert-butyl 2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carboxylate (52.0 mg, 0.08 mmol) in EtOH (1.00 mL) was added para-toluene sulfonate hydrate (31.1 mg, 0.16 mmol). The mixture was heated at 90° C. for ninety minutes. The mixture was partitioned between dilute aqueous sodium bicarbonate and 20% MeOH:DCM. The aqueous layer was extracted two more times with 20% MeOH:DCM. The combined organic layers were dried over Na₂SO₄, filtered, and concentrated to provide 2-{2-[(4aS,5aR)-5,5-difluoro-5a-methyl-1H,4H,4aH,6H-cyclopropa[f]indazol-3-yl)-1H-indol-6-yl}-2,8-diazaspiro[4.5]decan-1-one (0.0350 g, 94.8%). LCMS: C₂₅H₂₇F₂N₅O requires: 451, found: m/z=452 [M+H]⁺.

### Example 153. 5-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)-2-(2,6-dioxopiperidin-3-yl)isoindoline-1,3-dione (8)

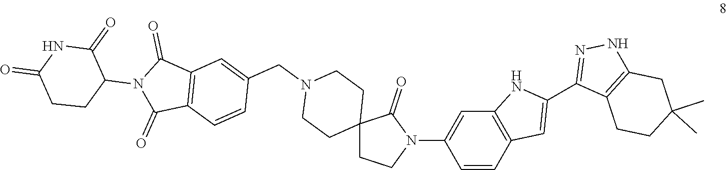 [SMILES: CC1(C)CCc2c(-c3cc4ccc(N5CCC6(CCN(Cc7ccc8c(c7)C(=O)N(C7CCC(=O)NC7=O)C8=O)CC6)C5=O)cc4[nH]3)n[nH]c2C1]

To a mixture of 2-[2-(6,6-dimethyl-1,4,5,7-tetrahydroindazol-3-yl)-1H-indol-6-yl]-2,8-diazaspiro[4.5]decan-1-one (13 mg, 0.03 mmol) in DCM (1.00 mL) was added N,N-diisopropylethylamine (0.04 mL, 0.26 mmol). The resulting mixture was added to 2-(2,6-dioxopiperidin-3-yl)-1,3-dioxoisoindoline-5-carbaldehyde (9.2 mg, 0.03 mmol) followed by sodium triacetoxyborohydride (14 mg, 0.06 mmol). After stirring overnight, an additional portion of 2-(2,6-dioxopiperidin-3-yl)-1,3-dioxoisoindoline-5-carbaldehyde (9.2 mg, 0.03 mmol) was added followed by sodium triacetoxyborohydride (14 mg, 0.06 mmol). After two hours, water was added and the mixture was extracted twice with DCM. The combined organic layers were concentrated then purified by prep-TLC eluted with 5% MeOH:DCM then re-purified by HPLC (5 to 95% MeCN in water with 0.1% TFA) to provide 5-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)-2-(2,6-dioxopiperidin-3-yl)isoindoline-1,3-dione (0.0026 g, 12%). ¹H NMR (500 MHz, DMSO-d₆) δ 11.26 (s, 1H), 11.17 (s, 1H), 9.65 (s, 1H), 8.15 (s, 1H), 8.12-8.06 (m, 1H), 7.99 (d, J=7.7 Hz, 1H), 7.75-7.69 (m, 1H), 7.49 (d, J=8.6 Hz, 1H), 7.24 (dd, J=8.7, 2.0 Hz, 1H), 6.56 (s, 1H), 5.21 (dd, J=12.9, 5.4 Hz, 1H), 4.61-4.56 (m, 1H), 3.92-3.83 (m, 2H), 3.47-3.41 (m, 2H), 3.21-3.14 (m, 2H), 2.92 (ddd, J=17.5, 14.1, 5.4 Hz, 1H), 2.70-2.64 (m, 3H), 2.44-2.40 (m, 1H), 2.19 (t, J=7.0 Hz, 2H), 2.13-1.97 (m, 3H), 1.88-1.82 (m, 2H), 1.58 (t, J=6.5 Hz, 3H), 1.38-1.27 (m, 1H), 1.02 (s, 6H), 0.95 (t, J=7.3 Hz, 1H). LCMS: C₃₉H₄₁N₇O₅ requires: 687, found: m/z=688 [M+H]⁺.

### Example 154. (2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-(((3R)-1-(2-(2,6-dioxopiperidin-3-yl)-1,3-dioxoisoindolin-5-yl)pyrrolidin-3-yl)methyl)piperazin-1-yl)-N-methylpropanamide (9)

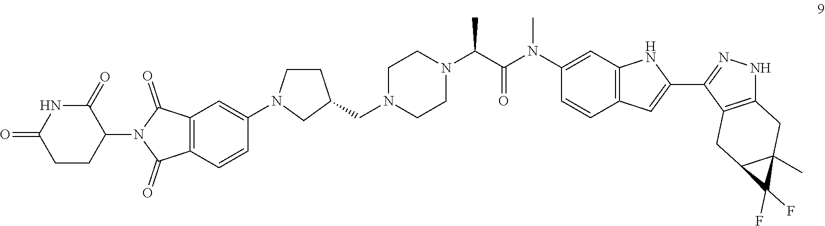 [SMILES: C[C@@H](C(=O)N(C)c1ccc2cc(-c3n[nH]c4c3C[C@@H]3C(F)(F)[C@]3(C)C4)[nH]c2c1)N1CCN(C[C@H]2CCN(c3ccc4c(c3)C(=O)N(C3CCC(=O)NC3=O)C4=O)C2)CC1]

tert-butyl 4-[(1S)-1-({2-[(4aS,5aR)-5,5-difluoro-5a-methyl-1-(oxan-2-yl)-4H,4aH,6H-cyclopropa[indazol-3-yl]-1H-indol-6-yl}(methyl)carbamoyl)ethyl]piperazine-1-carboxylate (60 mg, 0.09 mmol) was stirred in EtOH (1.00 mL) and 4 M HCl solution in dioxane (1.00 mL, 4.00 mmol) for twenty minutes. The mixture was heated at 50° C. for fifteen minutes and then was concentrated. DCM (1.00 mL) was added followed by N,N-diisopropylethylamine (0.06 mL, 0.36 mmol). (3S)-1-(2-(2,6-dioxopiperidin-3-yl)-1,3-dioxoisoindolin-5-yl)pyrrolidine-3-carbaldehyde (32 mg, 0.09 mmol) was added followed by sodium triacetoxyborohydride (38 mg, 0.18 mmol). After stirring overnight, the reaction was quenched with water and extracted twice with DCM. The combined organic layers were concentrated then purified by prep-HPLC (10 to 95% MeCN in water with 0.1% TFA) to provide (2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-(((3R)-1-(2-(2,6-dioxopiperidin-3-yl)-1,3-dioxoisoindolin-5-yl)pyrrolidin-3-yl)methyl)piperazin-1-yl)-N-methylpropanamide (0.0059 g, 7.7%). ¹H NMR (500 MHz, DMSO-d₆) δ 11.47 (s, 1H), 11.08 (s, 1H), 7.69 (d, J=8.4 Hz, 1H), 7.63 (d, J=8.4 Hz, 1H), 7.34 (s, 1H), 7.03-6.88 (m, 2H), 6.82 (d, J=8.7 Hz, 1H), 6.67 (s, 1H), 5.07 (dd, J=12.8, 5.4 Hz, 1H), 4.56-3.34 (m, 10H), 3.26 (s, 3H), 3.22-2.97 (m, 3H), 2.95-2.81 (m, 3H), 2.51 (d, J=2.9 Hz, 5H), 2.29-2.12 (m, 1H), 2.08-1.96 (m, 1H), 1.95-1.71 (m, 2H), 1.40 (s, 3H), 1.34-1.00 (m, 5H). LCMS: C₄₃H₄₇F₂N₉O₅ requires: 807, found: m/z=808 [M+H]⁺.

### Example 155. (2S)—N-(2-(6,6-dimethyl-4,5,6,7-tetrahydro-1H-indazol-3-yl)-1H-indol-6-yl)-2(4-((1-(5-(2,6-dioxopiperidin-3-yl)pyridin-2-yl)piperidin-4-yl)methyl)piperazin-1-yl)-N-methylpropanamide (10)

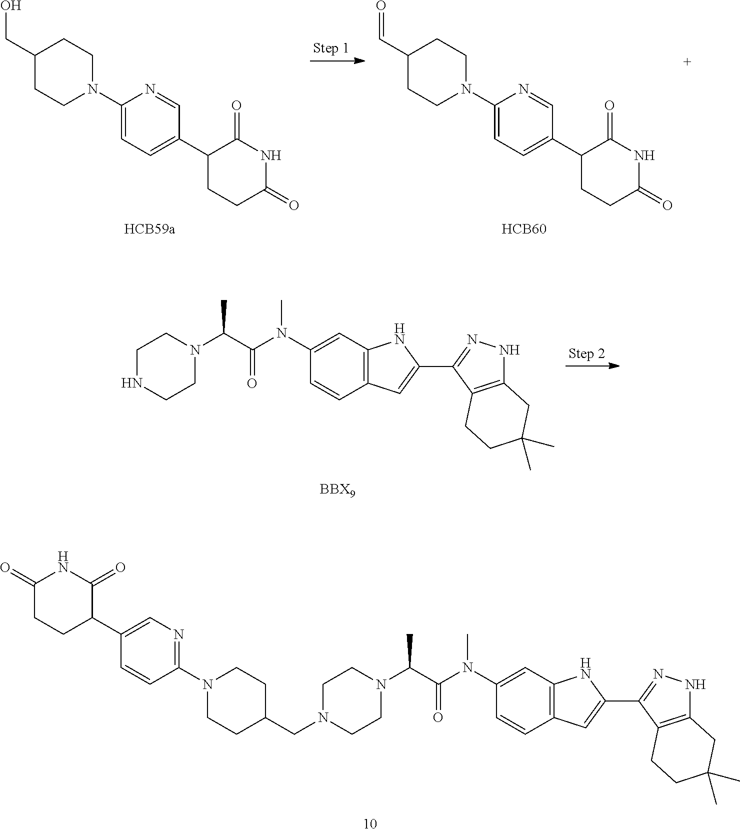

### Step 1: Synthesis of 1-(5-(2,6-dioxopiperidin-3-yl)pyridin-2-yl)piperidine-4-carbaldehyde (HCB60)

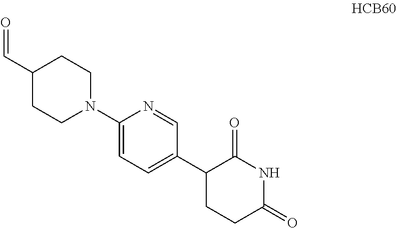 [SMILES: O=CC1CCN(c2ccc(C3CCC(=O)NC3=O)cn2)CC1]

To a mixture of 3-(6-(4-(hydroxymethyl)piperidin-1-yl)pyridin-3-yl)piperidine-2,6-dione (25 mg, 0.08 mmol) in DMSO (1.00 mL) was added triethylamine (0.23 mL, 0.17 g, 1.65 mmol) followed by sulfur trioxide pyridine complex (131 mg, 0.82 mmol). After fifteen minutes, TLC (10% MeOH:DCM) indicated complete consumption of the starting material. Water was added and the mixture was extracted twice with DCM. The combined organic layers were concentrated in vacuo to provide 1-(5-(2,6-dioxopiperidin-3-yl)pyridin-2-yl)piperidine-4-carbaldehyde as a mixture containing DMSO which was used without further purification. LCMS: C₁₆H₁₉N₃O₃ requires: 301, found: m/z=302 [M+H]⁺.

### Step 2: Synthesis of (2S)—N-(2-(6,6-dimethyl-4,5,6,7-tetrahydro-1H-indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (10)

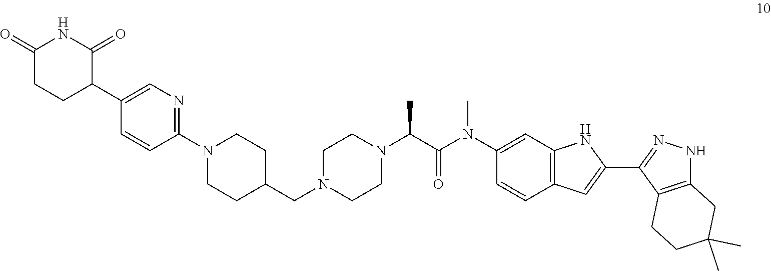 [SMILES: C[C@@H](C(=O)N(C)c1ccc2cc(-c3n[nH]c4c3CCC(C)(C)C4)[nH]c2c1)N1CCN(CC2CCN(c3ccc(C4CCC(=O)NC4=O)cn3)CC2)CC1]

To a mixture of (2S)—N-[2-(6,6-dimethyl-1,4,5,7-tetrahydroindazol-3-yl)-1H-indol-6-yl]-N-methyl-2-(piperazin-1-yl)propanamide (36 mg, 0.08 mmol) in DCM (1.00 mL) was added N,N-diisopropylethylamine (0.06 mL, 0.04 g, 0.33 mmol). The resulting mixture was added to 1-(5-(2,6-dioxopiperidin-3-yl)pyridin-2-yl)piperidine-4-carbaldehyde (25 mg, 0.08 mmol) followed by sodium triacetoxyborohydride (35 mg, 0.17 mmol). After one hour, water was added and the mixture was extracted twice with DCM. The combined organic layers were concentrated and the crude residue was purified by preparative HPLC (5 to 95% MeCN:water with 0.1% TFA) to provide (2S)—N-(2-(6,6-dimethyl-4,5,6,7-tetrahydro-1H-indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (0.0258 g, 43.3). ¹H NMR (500 MHz, DMSO-d₆) δ 11.44 (s, 1H), 10.89 (s, 1H), 9.05 (s, 1H), 7.92 (s, 1H), 7.61 (d, J=8.3 Hz, 1H), 7.33 (s, 1H), 7.12 (s, 1H), 6.95 (d, J=8.3 Hz, 1H), 6.65 (s, 1H), 4.26-4.20 (m, 2H), 3.87-3.81 (m, 1H), 3.61-3.32 (m, 3H), 3.25 (s, 3H), 3.10-2.78 (m, 7H), 2.75-2.64 (m, 3H), 2.61-2.34 (m, 11H), 2.25 (td, J=14.5, 14.0, 10.2 Hz, 1H), 2.01-1.93 (m, 1H), 1.84-1.77 (m, 2H), 1.62-1.56 (m, 2H), 1.33-1.07 (m, 4H), 1.02 (s, 6H). LCMS: C₄₁H₅₃N₉O₃ requires: 719, found: m/z=720 [M+H]⁺.

### Example 156. (S)—N-(2-(6,6-dimethyl-4,5,6,7-tetrahydro-1H-indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (11)

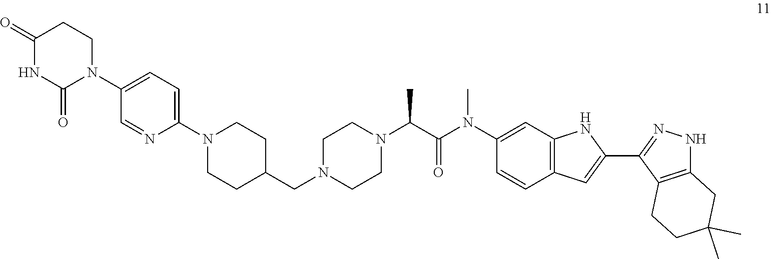 [SMILES: C[C@@H](C(=O)N(C)c1ccc2cc(-c3n[nH]c4c3CCC(C)(C)C4)[nH]c2c1)N1CCN(CC2CCN(c3ccc(N4CCC(=O)NC4=O)cn3)CC2)CC1]

(S)—N-(2-(6,6-dimethyl-4,5,6,7-tetrahydro-1H-indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (0.0248 g, 37.8%) was prepared by procedures analogous to Example 91 starting from 1-{6-[4-(hydroxymethyl)piperidin-1-yl]pyridin-3-yl}-1,3-diazinane-2,4-dione (28 mg, 0.09 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (40 mg, 0.09 mmol). ¹H NMR (500 MHz, DMSO-d₆) δ 11.44 (s, 1H), 10.37 (s, 1H), 8.06 (d, J=2.6 Hz, 1H), 7.61 (d, J=8.3 Hz, 1H), 7.55 (dd, J=9.1, 2.6 Hz, 1H), 7.32 (s, 1H), 6.98-6.90 (m, 2H), 6.65 (d, J=2.1 Hz, 1H), 4.28 (d, J=13.0 Hz, 2H), 3.71 (t, J=6.7 Hz, 2H), 3.60-3.32 (m, 2H), 3.25 (s, 3H), 2.91-2.82 (m, 3H), 2.75-2.65 (m, 4H), 2.51 (t, J=1.9 Hz, 11H), 2.03-1.99 (m, 1H), 1.80-1.74 (m, 2H), 1.59 (t, J=6.4 Hz, 2H), 1.18-1.15 (m, 4H), 1.02 (s, 6H). LCMS: C₄₀H₅₂N₁₀O₃ requires: 720, found: m/z=721 [M+H]⁺.

### Example 157. (2S)—N-(2-(6,6-dimethyl-4,5,6,7-tetrahydro-1H-indazol-3-yl)-1H-indol-6-yl)-2-(4-(((3R)-1-(2-(2,6-dioxopiperidin-3-yl)-1-oxoisoindolin-5-yl)pyrrolidin-3-yl)methyl)piperazin-1-yl)-N-methylpropanamide (12)

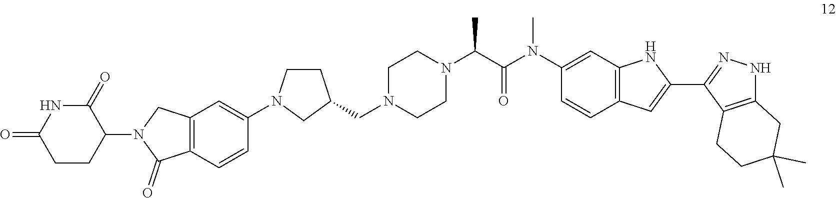 [SMILES: C[C@@H](C(=O)N(C)c1ccc2cc(-c3n[nH]c4c3CCC(C)(C)C4)[nH]c2c1)N1CCN(C[C@H]2CCN(c3ccc4c(c3)CN(C3CCC(=O)NC3=O)C4=O)C2)CC1]

(2S)—N-(2-(6,6-dimethyl-4,5,6,7-tetrahydro-1H-indazol-3-yl)-1H-indol-6-yl)-2-(4-(((3R)-1-(2-(2,6-dioxopiperidin-3-yl)-1-oxoisoindolin-5-yl)pyrrolidin-3-yl)methyl)piperazin-1-yl)-N-methylpropanamide (0.0244 g, 35.1%) was prepared by procedures analogous to Example 91 starting from (3RS)-3-{5-[(3S)-3-(hydroxymethyl)pyrrolidin-1-yl]-1-oxo-3H-isoindol-2-yl}piperidine-2,6-dione (30.0 mg, 0.09 mmol) and (S)—N-(2-(6,6-dimethyl-4,5,6,7-tetrahydro-1H-indazol-3-yl)-1H-indol-6-yl)-N-methyl-2-(piperazin-1-yl)propanamide (37.8 mg, 0.09 mmol). ¹H NMR (500 MHz, DMSO-d₆) δ 11.44 (s, 1H), 10.95 (s, 1H), 9.18 (s, 1H), 7.61 (d, J=8.3 Hz, 1H), 7.52 (d, J=8.8 Hz, 1H), 7.33 (s, 1H), 6.96 (d, J=8.3 Hz, 1H), 6.67-6.61 (m, 3H), 5.05 (dd, J=13.3, 5.2 Hz, 1H), 4.32 (d, J=16.6 Hz, 1H), 4.20 (d, J=16.7 Hz, 1H), 3.61-3.38 (m, 4H), 3.38-3.17 (m, 4H), 3.17-3.04 (m, 1H), 2.97-2.86 (m, 2H), 2.81-2.30 (m, 14H), 2.19 (s, 1H), 2.00-1.94 (m, 1H), 1.78 (s, 1H), 1.60 (t, J=6.4 Hz, 2H), 1.34-1.09 (m, 4H), 1.02 (s, 6H). LCMS: C₄₃H₅₃N₉O₄ requires: 759, found: m/z=760 [M+H]⁺.

### Example 158. 3-(4-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)-3-methyl-2-oxo-2,3-dihydro-1H-benzo[d]imidazol-1-yl)piperidine-2,6-dione (13)

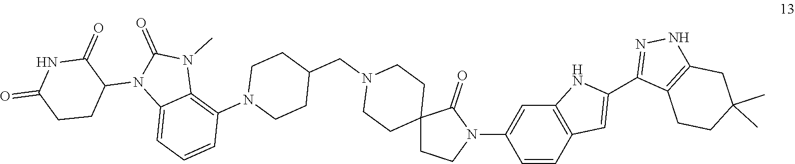 [SMILES: Cn1c(=O)n(C2CCC(=O)NC2=O)c2cccc(N3CCC(CN4CCC5(CC4)CCN(c4ccc6cc(-c7n[nH]c8c7CCC(C)(C)C8)[nH]c6c4)C5=O)CC3)c21]

3-(4-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)-3-methyl-2-oxo-2,3-dihydro-1H-benzo[d]imidazol-1-yl)piperidine-2,6-dione (0.0126 g, 23.3%) was prepared by procedures analogous to Example 91 starting from 3-(4-(4-(hydroxymethyl)piperidin-1-yl)-3-methyl-2-oxo-2,3-dihydro-1H-benzo[d]imidazol-1-yl)piperidine-2,6-dione (25.0 mg, 0.07 mmol) and 2-[2-(6,6-dimethyl-1,4,5,7-tetrahydroindazol-3-yl)-1H-indol-6-yl]-2,8-diazaspiro[4.5]decan-1-one (25.1 mg, 0.06 mmol). ¹H NMR (500 MHz, DMSO-d₆) δ 12.52 (s, 1H), 11.27 (s, 1H), 11.11 (s, 1H), 9.12 (s, 1H), 7.76 (s, 1H), 7.51 (d, J=8.5 Hz, 1H), 7.25 (d, J=8.6 Hz, 1H), 7.01 (t, J=7.9 Hz, 1H), 6.95-6.88 (m, 2H), 6.56 (s, 1H), 5.38 (dd, J=12.8, 5.5 Hz, 1H), 3.93-3.86 (m, 2H), 3.66 (s, 3H), 3.64-3.57 (m, 1H), 3.24-3.02 (m, 6H), 2.96-2.86 (m, 1H), 2.81-2.60 (m, 6H), 2.42 (s, 2H), 2.25-2.12 (m, 3H), 2.12-1.78 (m, 7H), 1.59 (t, J=6.3 Hz, 2H), 1.51-1.45 (m, 2H), 1.25 (s, 1H), 1.02 (s, 6H). LCMS: C₄₄H₅₃N₉O₄ requires: 771, found: m/z=772 [M+H]⁺.

### Example 159. 3-(5-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)-3-methyl-2-oxo-2,3-dihydro-1H-benzo[d]imidazol-1-yl)piperidine-2,6-dione (14)

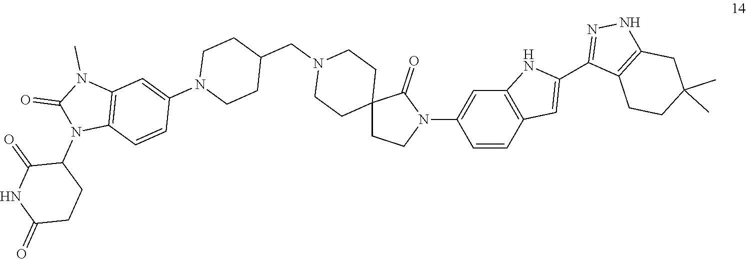 [SMILES: Cn1c(=O)n(C2CCC(=O)NC2=O)c2ccc(N3CCC(CN4CCC5(CC4)CCN(c4ccc6cc(-c7n[nH]c8c7CCC(C)(C)C8)[nH]c6c4)C5=O)CC3)cc21]

3-(5-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)-3-methyl-2-oxo-2,3-dihydro-1H-benzo[d]imidazol-1-yl)piperidine-2,6-dione (0.0178 g, 32.6%) was prepared by procedures analogous to Example 91 starting from 3-(5-(4-(hydroxymethyl)piperidin-1-yl)-3-methyl-2-oxo-2,3-dihydro-1H-benzo[d]imidazol-1-yl)piperidine-2,6-dione (25.0 mg, 0.07 mmol) and 2-[2-(6,6-dimethyl-1,4,5,7-tetrahydroindazol-3-yl)-1H-indol-6-yl]-2,8-diazaspiro[4.5]decan-1-one (20.0 mg, 0.05 mmol). ¹H NMR (500 MHz, DMSO-d₆) δ 12.51 (s, 1H), 11.28 (s, 1H), 11.08 (s, 1H), 9.18 (s, 1H), 7.77-7.71 (m, 1H), 7.50 (d, J=8.6 Hz, 1H), 7.28-7.22 (m, 1H), 6.96 (s, 1H), 6.87 (s, 1H), 6.67 (s, 1H), 6.56 (s, 1H), 5.34-5.30 (m, 1H), 3.93-3.85 (m, 2H), 3.68-3.57 (m, 4H), 3.10 (t, J=10.5 Hz, 4H), 2.93-2.86 (m, 1H), 2.78-2.59 (m, 5H), 2.58-2.35 (m, 4H), 2.23-2.13 (m, 4H), 2.09-1.79 (m, 7H), 1.59 (t, J=6.4 Hz, 2H), 1.42-1.39 (m, 3H), 1.25 (s, 1H), 1.02 (s, 6H). LCMS: C₄₄H₅₃N₉O₄ requires: 771, found: m/z=772 [M+H]⁺.

### Example 160. 5-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)-2-(2,6-dioxopiperidin-3-yl)isoindoline-1,3-dione hydrochloride (15)

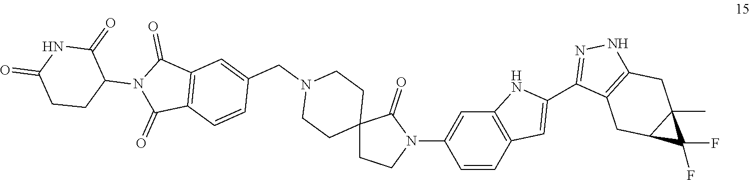 [SMILES: C[C@@]12Cc3[nH]nc(-c4cc5ccc(N6CCC7(CCN(Cc8ccc9c(c8)C(=O)N(C8CCC(=O)NC8=O)C9=O)CC7)C6=O)cc5[nH]4)c3C[C@@H]1C2(F)F]

A mixture of 2-{2-[(4aS,5aR)-5,5-difluoro-5a-methyl-1H,4H,4aH,6H-cyclopropa[f]indazol-3-yl]-1H-indol-6-yl}-2,8-diazaspiro[4.5]decan-1-one (25.0 mg, 0.06 mmol), 5-(bromomethyl)-2-(2,6-dioxopiperidin-3-yl)isoindoline-1,3-dione (25.0 mg, 0.07 mmol) and N,N-diisopropylethylamine (0.05 mL, 0.04 g, 0.28 mmol) in DMF (1.00 mL) were heated at 50° C. for thirty minutes. The mixture was concentrated in vacuo then purified by preparative TLC eluted with 8% MeOH:DCM. The resulting material was suspended MeCN (2 mL) and water (2 mL) and 0.1 N aq. HCl (1 equiv) was added. The mixture went homogeneous and was frozen and lyophilized to provide 5-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)-2-(2,6-dioxopiperidin-3-yl)isoindoline-1,3-dione hydrochloride (0.0126 g, 30.6%). ¹H NMR (500 MHz, DMSO-d₆) δ 12.64 (s, 1H), 11.29 (s, 1H), 11.16 (s, 1H), 10.06 (s, 1H), 8.19 (s, 1H), 8.08 (d, J=7.9 Hz, 1H), 8.04 (d, J=7.8 Hz, 1H), 7.75 (s, 1H), 7.51 (d, J=8.6 Hz, 1H), 7.28-7.21 (m, 1H), 6.57 (s, 1H), 5.21 (dd, J=12.8, 5.4 Hz, 1H), 4.63-4.56 (m, 2H), 3.92-3.83 (m, 2H), 3.23-2.97 (m, 5H), 2.97-2.79 (m, 2H), 2.72-2.34 (m, 3H), 2.19 (t, J=7.0 Hz, 2H), 2.13-1.92 (m, 4H), 1.90-1.81 (m, 3H), 1.40 (s, 3H). LCMS: C₃₉H₃₇F₂N₇O₅ requires: 721, found: m/z=722 [M+H]⁺.

### Example 161. 3-(4-(4-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)piperidin-1-yl)-3-methyl-2-oxo-2,3-dihydro-1H-benzo[d]imidazol-1-yl)piperidine-2,6-dione (16)

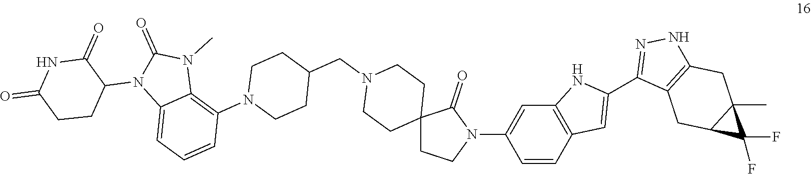 [SMILES: Cn1c(=O)n(C2CCC(=O)NC2=O)c2cccc(N3CCC(CN4CCC5(CC4)CCN(c4ccc6cc(-c7n[nH]c8c7C[C@@H]7C(F)(F)[C@]7(C)C8)[nH]c6c4)C5=O)CC3)c21]

3-(4-(4-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)piperidin-1-yl)-3-methyl-2-oxo-2,3-dihydro-1H-benzo[d]imidazol-1-yl)piperidine-2,6-dione (0.0103 g, 25.0%) was prepared by procedures analogous to Example 91 starting from 3-(4-(4-(hydroxymethyl)piperidin-1-yl)-3-methyl-2-oxo-2,3-dihydro-1H-benzo[d]imidazol-1-yl)piperidine-2,6-dione (18.0 mg, 0.05 mmol) and 2-{2-[(4aS,5aR)-5,5-difluoro-5a-methyl-1H,4H,4aH,6H-cyclopropa[f]indazol-3-yl)-1H-indol-6-yl}-2,8-diazaspiro[4.5]decan-1-one (25.0 mg, 0.06 mmol). ¹H NMR (500 MHz, DMSO-d₆) δ 12.65 (s, 1H), 11.31 (s, 1H), 11.10 (s, 1H), 9.17 (s, 1H), 7.78-7.72 (m, 1H), 7.52 (d, J=8.6 Hz, 1H), 7.27 (d, J=8.5 Hz, 1H), 7.04-6.96 (m, 1H), 6.95-6.88 (m, 2H), 6.57 (s, 1H), 5.37 (dd, J=12.8, 5.5 Hz, 1H), 3.90 (q, J=7.0 Hz, 2H), 3.67-3.57 (m, 5H), 3.50-3.46 (m, 1H), 3.22-2.98 (m, 7H), 2.96-2.58 (m, 5H), 2.23-2.13 (m, 3H), 2.09-1.74 (m, 8H), 1.57-1.35 (m, 5H), 1.33-1.12 (m, 2H). LCMS: C₄₄H₄₉F₂N₉O₄ requires: 805, found: m/z=806 [M+H]⁺.

### Example 162. (2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (17)

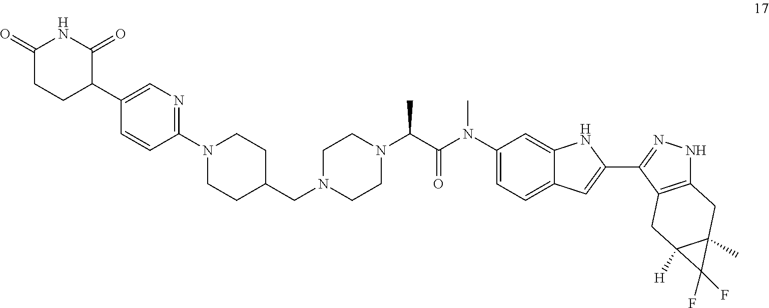 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(C4CCC(=O)NC4=O)cn3)CC2)CC1]

(2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,6-dioxopiperidin-3-yl)pyridin-2-yl)piperidin-4-yl)methyl)piperazin-1-yl)-N-methylpropanamide (0.0266 g, 34.6%) was prepared by procedures analogous to Example 91 starting from 3-(6-(4-(hydroxymethyl)piperidin-1-yl)pyridin-3-yl)piperidine-2,6-dione (30.0 mg, 0.10 mmol) and (2S)—N-{2-[(4aS,5aR)-5,5-difluoro-5a-methyl-1H,4H,4aH,6H-cyclopropa[ ]indazol-3-yl]-1H-indol-6-yl}-N-methyl-2-(piperazin-1-yl)propanamide (46.9 mg, 0.10 mmol). ¹HNMR (500 MHz, DMSO-d₆) δ 12.72 (s, 1H), 11.46 (s, 1H), 10.82 (s, 1H), 9.16 (s, 1H), 7.97-7.91 (m, 1H), 7.59 (d, J=8.3 Hz, 1H), 7.40-7.36 (m, 1H), 7.31 (s, 1H), 6.93 (d, J=8.3 Hz, 1H), 6.86-6.75 (m, 1H), 6.64 (s, 1H), 4.31-4.24 (m, 2H), 3.74 (dd, J=12.2, 5.0 Hz, 1H), 3.42 (t, J=11.7 Hz, 2H), 3.22 (s, 3H), 3.18-2.74 (m, 12H), 2.74-2.58 (m, 3H), 2.22-2.14 (m, 1H), 2.12-1.91 (m, 2H), 1.92-1.83 (m, 1H), 1.83-1.67 (m, 3H), 1.42-1.35 (m, 4H), 1.28-1.13 (m, 2H), 1.08 (d, J=6.8 Hz, 3H). LCMS: C₄₁H₄₉F₂N₉O₃ requires: 753, found: m/z=754 [M+H]⁺.

### Example 163. (S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (18)

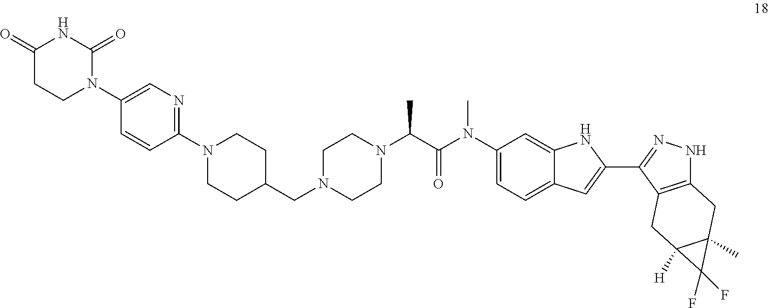 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(N4CCC(=O)NC4=O)cn3)CC2)CC1]

(S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(5-(2,4-dioxotetrahydropyrimidin-1(2H)-yl)pyridin-2-yl)piperidin-4-yl)methyl)piperazin-1-yl)-N-methylpropanamide (0.0250 g, 32.1%) was prepared by procedures analogous to Example 91 starting from 1-{6-[4-(hydroxymethyl)piperidin-1-yl]pyridin-3-yl}-1,3-diazinane-2,4-dione (30.0 mg, 0.10 mmol) and (2S)—N-{2-[(4aS,5aR)-5,5-difluoro-5a-methyl-1H,4H,4aH,6H-cyclopropa[ ]indazol-3-yl]-1H-indol-6-yl}-N-methyl-2-(piperazin-1-yl)propanamide (46.9 mg, 0.10 mmol). ¹H NMR (500 MHz, DMSO-d₆) δ 12.73 (s, 1H), 11.47 (s, 1H), 10.35 (s, 1H), 9.22 (s, 1H), 8.08-8.02 (m, 1H), 7.60 (d, J=8.3 Hz, 1H), 7.53-7.47 (m, 1H), 7.32 (s, 1H), 6.96-6.86 (m, 2H), 6.65 (s, 1H), 4.29 (d, J=13.3 Hz, 2H), 3.70 (t, J=6.7 Hz, 2H), 3.53-3.25 (m, 3H), 3.22 (s, 3H), 3.19-2.74 (m, 12H), 2.74-2.59 (m, 3H), 2.07-2.04 (m, 1H), 1.92-1.84 (m, 1H), 1.83-1.67 (m, 3H), 1.45-1.32 (m, 4H), 1.22-1.18 (m, 2H), 1.09 (d, J=6.7 Hz, 3H). LCMS: C₄₀H₄₈F₂N₁₀O₃ requires: 754, found: m/z=755 [M+H]⁺.

### Example 164. 3-(6-(4-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)piperidin-1-yl)pyridin-3-yl)piperidine-2,6-dione (19)

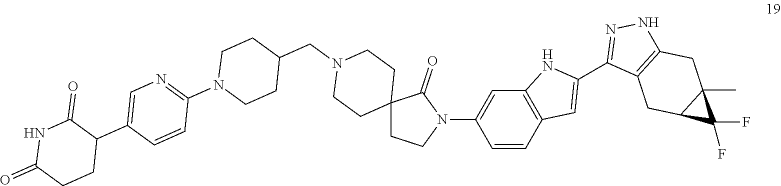 [SMILES: C[C@@]12Cc3[nH]nc(-c4cc5ccc(N6CCC7(CCN(CC8CCN(c9ccc(C%10CCC(=O)NC%10=O)cn9)CC8)CC7)C6=O)cc5[nH]4)c3C[C@@H]1C2(F)F]

3-(6-(4-((2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)methyl)piperidin-1-yl)pyridin-3-yl)piperidine-2,6-dione (0.0149 g, 33.4%) was prepared by procedures analogous to Example 91 starting from 3-(6-(4-(hydroxymethyl)piperidin-1-yl)pyridin-3-yl)piperidine-2,6-dione (18.7 mg, 0.06 mmol) and 2-{2-[(4aS,5aR)-5,5-difluoro-5a-methyl-1H,4H,4aH,6H-cyclopropa[indazol-3-yl]-1H-indol-6-yl}-2,8-diazaspiro[4.5]decan-1-one (22.0 mg, 0.05 mmol). ¹H NMR (500 MHz, DMSO-d₆) δ 12.65 (s, 1H), 11.30 (s, 1H), 10.85 (s, 1H), 9.16 (s, 1H), 7.97-7.93 (m, 1H), 7.78-7.72 (m, 1H), 7.54-7.48 (m, 1H), 7.39 (s, 1H), 7.26 (dd, J=8.4, 2.0 Hz, 1H), 6.90 (s, 1H), 6.59 (d, J=17.2 Hz, 1H), 4.31 (d, J=13.0 Hz, 2H), 3.93-3.85 (m, 2H), 3.79 (s, 1H), 3.62-3.56 (m, 2H), 3.48-3.44 (m, 1H), 3.17-2.97 (m, 7H), 2.97-2.80 (m, 1H), 2.75-2.65 (m, 1H), 2.57-2.53 (m, 2H), 2.22-2.12 (m, 5H), 2.02-1.95 (m, 1H), 1.89-1.80 (m, 5H), 1.40 (s, 3H), 1.27-1.23 (m, 3H). LCMS: C₄1H₄₆F₂N₈O₃ requires: 736, found: m/z=737 [M+H]⁺.

### Example 165. N-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-2-(1-(5-(2,6-dioxopiperidin-3-yl)pyridin-2-yl)piperidin-4-yl)-N-methylacetamide (20)

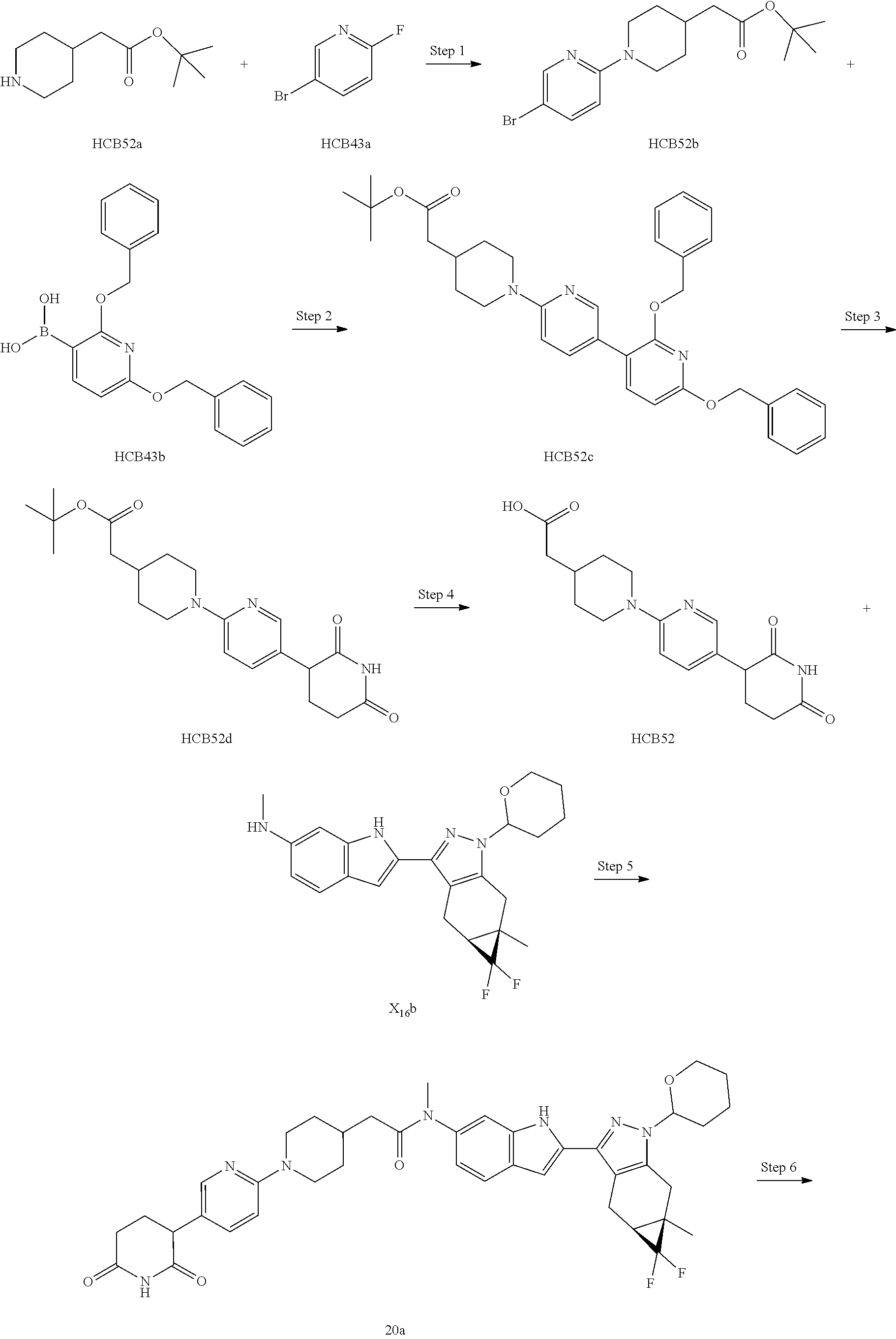

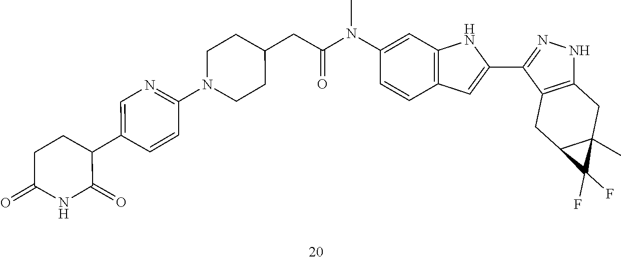 [SMILES: CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1]

### Step 1: tert-butyl 2-[1-(5-bromopyridin-2-yl)piperidin-4-yl] acetate (HCB52b)

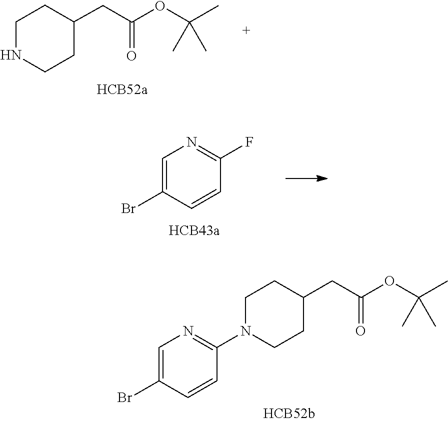 [SMILES: CC(C)(C)OC(=O)CC1CCNCC1 ; Fc1ccc(Br)cn1 ; CC(C)(C)OC(=O)CC1CCN(c2ccc(Br)cn2)CC1]

tert-butyl 2-(piperidin-4-yl)acetate (765 mg, 3.84 mmol), 5-bromo-2-fluoropyridine (0.40 mL, 676 mg, 3.84 mmol), and potassium carbonate (1.06 g, 7.68 mmol) were stirred in DMF (10.00 mL) overnight. The mixture was heated at 50° C. for six hours. The mixture was stirred at room temperature for five days, transferred to a separatory funnel with ethyl acetate, and then washed with two portions of water. The organic layer was dried over Na₂SO₄, filtered, and concentrated. The crude residue was purified by flash chromatography on a 40 g column e(gradient elution with zero to 20% ethyl acetate:hexanes) to provide tert-butyl 2-[1-(5-bromopyridin-2-yl)piperidin-4-yl]acetate (0.655 g, 48.0%). LCMS: C₁₆H₂₃BrN₂O₂ requires: 355, found: m/z=356 [M+H]⁺.

### Step 2: tert-butyl 2-{1-[2′,6′-bis(benzyloxy)-[3,3′-bipyridin]-6-yl]piperidin-4-yl}acetate (HCB52c)

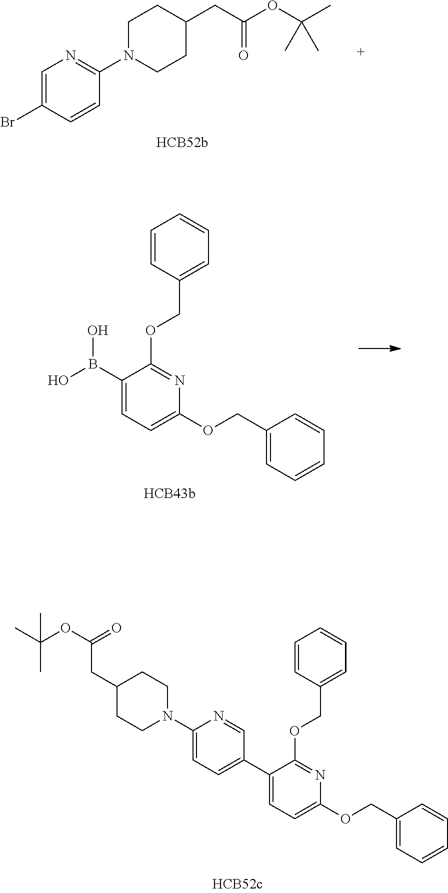

A mixture of 2,6-bis(benzyloxy)pyridin-3-ylboronic acid (495 mg, 1.48 mmol), tert-butyl 2-[1-(5-bromopyridin-2-yl)piperidin-4-yl]acetate (525 mg, 1.48 mmol), tetrakis(triphenylphosphine)palladium(0) (171 mg, 0.15 mmol), and potassium carbonate (408 mg, 2.96 mmol) in water (1.00 mL) and THE (3.00 mL) was microwaved at 120° C. for forty minutes. The water layer was removed by pipette. The organic layer was concentrated and then purified by flash chromatography on a 40 g column (gradient elution with zero to 35% ethyl acetate:hexanes) to provide tert-butyl 2-{1-[2′,6′-bis(benzyloxy)-[3,3′-bipyridin]-6-yl]piperidin-4-yl}acetate (0.303 g, 36.2%). LCMS: C₃₅H₃₉N₃O₄ requires: 565, found: m/z=566 [M+H]⁺.

### Step 3: tert-butyl 2-{1-[5-(2,6-dioxopiperidin-3-yl)pyridin-2-yl]piperidin-4-yl}acetate (HCB52d)

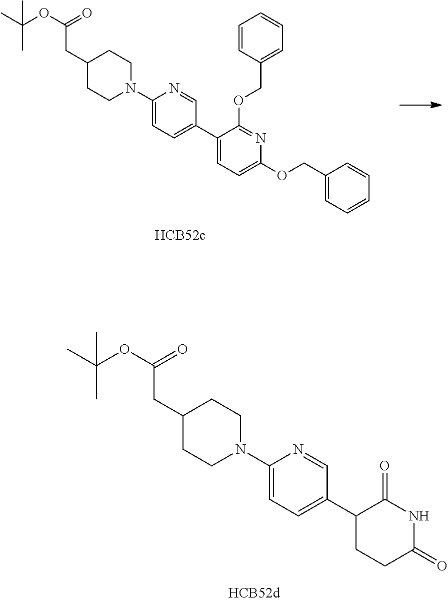 [SMILES: CC(C)(C)OC(=O)CC1CCN(c2ccc(-c3ccc(OCc4ccccc4)nc3OCc3ccccc3)cn2)CC1 ; CC(C)(C)OC(=O)CC1CCN(c2ccc(C3CCC(=O)NC3=O)cn2)CC1]

tert-butyl 2-{1-[2′,6′-bis(benzyloxy)-[3,3′-bipyridin]-6-yl]piperidin-4-yl}acetate (303 mg, 0.54 mmol) was suspended in EtOH (9.00 mL). THE (4 mL) was added along with 10% Pd/C (303 mg). The mixture was stirred under a balloon of H₂ for two hours. The mixture was diluted with THF (100 mL) and filtered through a pad of celite. The filtrate was concentrated and the crude residue was purified by flash chromatography on a 24 g column (gradient elution with zero to 10% MeOH:DCM) to provide tert-butyl 2-{1-[5-(2,6-dioxopiperidin-3-yl)pyridin-2-yl]piperidin-4-yl}acetate (0.152 g, 73.2%). LCMS: C₂₁H₂₉N₃O₄ requires: 387, found: m/z=388 [M+H]⁺.

### Step 4: {1-[5-(2,6-dioxopiperidin-3-yl)pyridin-2-yl]piperidin-4-yl}acetic acid (HCB52)

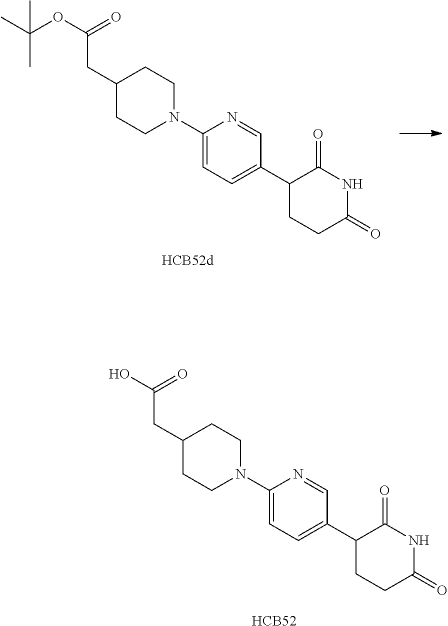 [SMILES: CC(C)(C)OC(=O)CC1CCN(c2ccc(C3CCC(=O)NC3=O)cn2)CC1 ; "HCB52" ; O=C(O)CC1CCN(c2ccc(C3CCC(=O)NC3=O)cn2)CC1]

tert-butyl 2-{1-[5-(2,6-dioxopiperidin-3-yl)pyridin-2-yl]piperidin-4-yl}acetate (29.00 mg, 0.07 mmol) was stirred in DCM (0.50 mL) and 4 M HCl solution in dioxane (0.50 mL, 0.07 g, 2.00 mmol). After stirring overnight, the mixture was concentrated in vacuo to provide {1-[5-(2,6-dioxopiperidin-3-yl)pyridin-2-yl]piperidin-4-yl}acetic acid (0.0248 g, 100%). LCMS: C₁₇H₂₁N₃O₄ requires: 331, found: m/z=332 [M+H]⁺.

### Step 5: N-{2-[(4aS,5aR)-5,5-difluoro-5a-methyl-1-(oxan-2-yl)-4H,4aH,6H-cyclopropa[f]indazol-3-yl]-1H-indol-6-yl}-2-(1-{5-[(3RS)-2,6-dioxopiperidin-3-yl]pyridin-2-yl}piperidin-4-yl)-N-methylacetamide (20a)

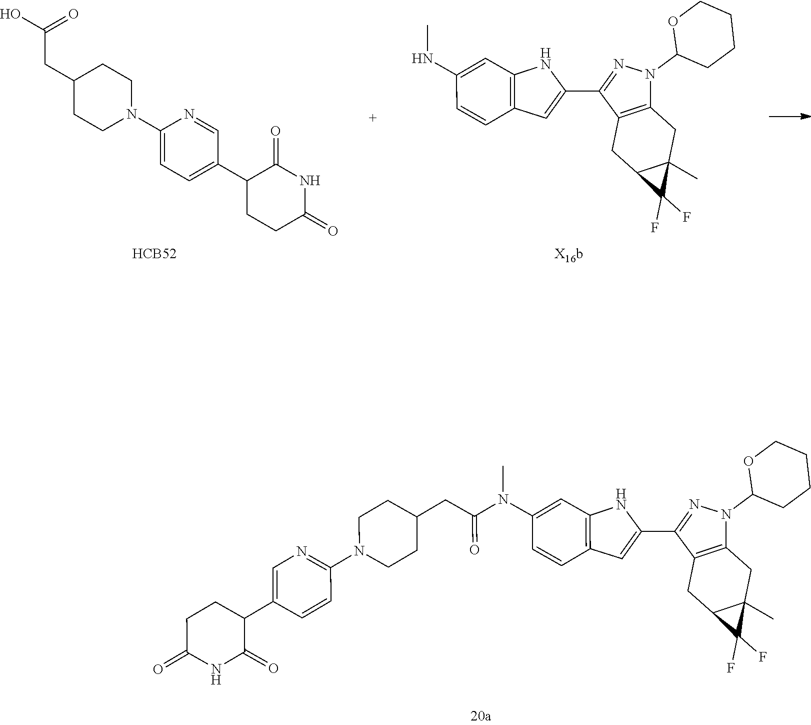 [SMILES: "HCB52" ; O=C(O)CC1CCN(c2ccc(C3CCC(=O)NC3=O)cn2)CC1 ; CNc1ccc2cc(-c3nn(C4CCCCO4)c4c3C[C@@H]3C(F)(F)[C@]3(C)C4)[nH]c2c1 ; "X16b" ; CN(C(=O)CC1CCN(c2ccc(C3CCC(=O)NC3=O)cn2)CC1)c1ccc2cc(-c3nn(C4CCCCO4)c4c3C[C@@H]3C(F)(F)[C@]3(C)C4)[nH]c2c1]

2-[(4aS,5aR)-5,5-difluoro-5a-methyl-1-(oxan-2-yl)-4H,4aH,6H-cyclopropa[f]indazol-3-yl]-N-methyl-1H-indol-6-amine (28.9 mg, 0.07 mmol) in DMF (2.00 mL) was added to a mixture of 2-(1-(5-(2,6-dioxopiperidin-3-yl)pyridin-2-yl)piperidin-4-yl)acetic acid (23.2 mg, 0.07 mmol), HATU (26.6 mg, 0.07 mmol), and N,N-diisopropylethylamine (0.05 mL, 0.04 g, 0.28 mmol) in DMF (2.00 mL). After stirring overnight, water was added and the mixture was extracted with ethyl acetate. The organic layer was concentrated and the crude residue was purified by preparative TLC eluted with 5% MeOH:DCM to provide N-{2-[(4aS,5aR)-5,5-difluoro-5a-methyl-1-(oxan-2-yl)-4H,4aH,6H-cyclopropa[f]indazol-3-yl]-1H-indol-6-yl}-2-(1-{5-[(3RS)-2,6-dioxopiperidin-3-yl]pyridin-2-yl}piperidin-4-yl)-N-methylacetamide (0.0428 g, 84.2%). LCMS: C₄₀H₄₅F₂N₇O₄ requires: 725, found: m/z=726 [M+H]⁺.

### Step 6: N-(2-((4aS,5aR)-5,5-difluoro-5a-methyl-1-(tetrahydro-2H-pyran-2-yl)-1,4,4a,5,5a,6-hexahydrocyclopropa[ ]indazol-3-yl)-1H-indol-6-yl)-2-(1-(5-(2,6-dioxopiperidin-3-yl)pyridin-2-yl)piperidin-4-yl)-N-methylacetamide (20)

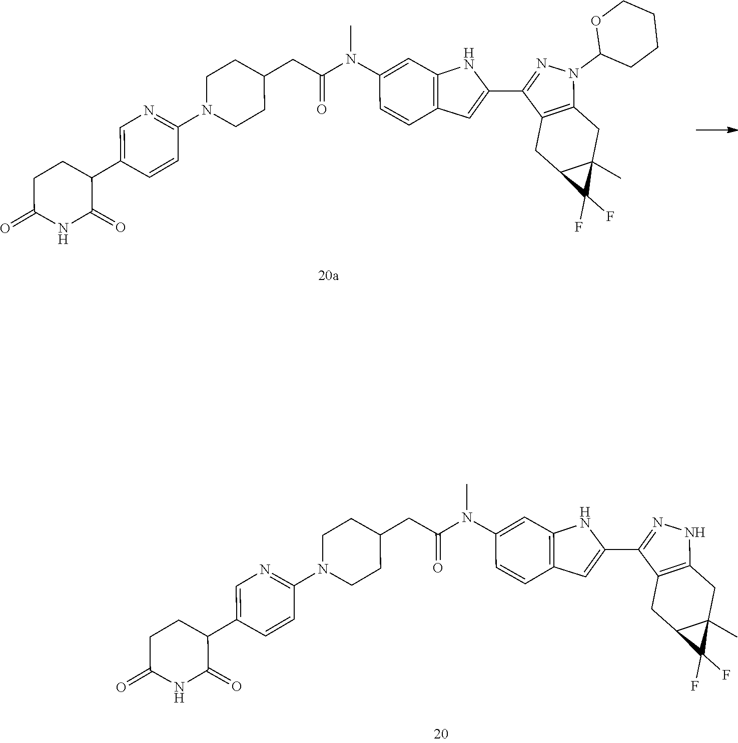 [SMILES: CN(C(=O)CC1CCN(c2ccc(C3CCC(=O)NC3=O)cn2)CC1)c1ccc2cc(-c3nn(C4CCCCO4)c4c3C[C@@H]3C(F)(F)[C@]3(C)C4)[nH]c2c1 ; CN(C(=O)CC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1]

N-{2-[(4aS,5aR)-5,5-difluoro-5a-methyl-1-(oxan-2-yl)-4H,4aH,6H-cyclopropa[f]indazol-3-yl]-1H-indol-6-yl}-2-(1-{5-[(3RS)-2,6-dioxopiperidin-3-yl]pyridin-2-yl}piperidin-4-yl)-N-methylacetamide (42.8 mg, 0.06 mmol) and para-toluene sulfonate hydrate (0.02 mL, 22.4 mg, 0.12 mmol) were stirred in EtOH (2.00 mL) at 50° C. for one hour. The mixture was diluted with water then extracted twice with 10% MeOH:DCM. The combined organic layers were concentrated and the crude residue was purified by preparative TLC eluted with 10% MeOH:DCM to provide N-(2-((4aS,5aR)-5,5-difluoro-5a-methyl-1-(tetrahydro-2H-pyran-2-yl)-1,4,4a,5,5a,6-hexahydrocyclopropa[f]indazol-3-yl)-1H-indol-6-yl)-2-(1-(5-(2,6-dioxopiperidin-3-yl)pyridin-2-yl)piperidin-4-yl)-N-methylacetamide (0.0227 g, 59.4%). ¹H NMR (500 MHz, DMSO-d₆) δ 12.67 (s, 1H), 11.43 (s, 1H), 10.79 (s, 1H), 7.94-7.88 (m, 1H), 7.56 (d, J=8.2 Hz, 1H), 7.35-7.29 (m, 1H), 7.23 (s, 1H), 6.87 (d, J=8.3 Hz, 1H), 6.73 (d, J=8.7 Hz, 1H), 6.62 (s, 1H), 4.17 (d, J=12.8 Hz, 2H), 3.69 (dd, J=12.0, 4.9 Hz, 1H), 3.56 (d, J=3.5 Hz, 1H), 3.21 (s, 3H), 3.17-2.96 (m, 3H), 2.87-2.80 (m, 1H), 2.79-2.58 (m, 3H), 2.20-2.08 (m, 1H), 2.04-1.91 (m, 4H), 1.86 (dd, J=15.1, 6.9 Hz, 1H), 1.65 (d, J=12.7 Hz, 2H), 1.39 (s, 3H), 0.98-0.92 (m, 2H). LCMS: C₃₅H₃₇F₂N₇O₃ requires: 641, found: m/z=642 [M+H]⁺.

### Example 166. N-(2-((4aS,5aR)-5,5-difluoro-5a-methyl-1,4,4a,5,5a,6-hexahydrocyclopropa[f]indazol-3-yl)-1H-indol-6-yl)-3-(1-(5-(2,6-dioxopiperidin-3-yl)pyridin-2-yl)piperidin-4-yl)-N-methylpropanamide (21)

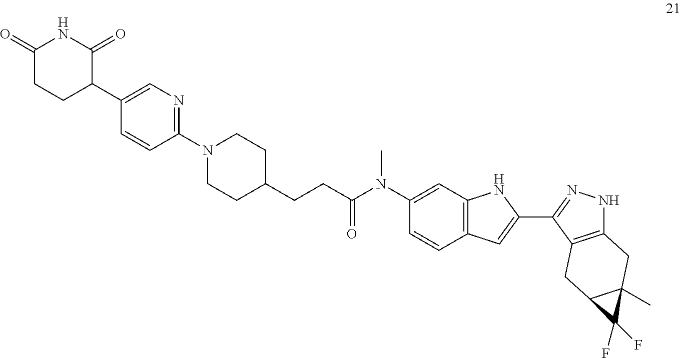 [SMILES: CN(C(=O)CCC1CCN(c2ccc(C3CCC(=O)NC3=O)cn2)CC1)c1ccc2cc(-c3n[nH]c4c3C[C@@H]3C(F)(F)[C@]3(C)C4)[nH]c2c1]

N-(2-((4aS,5aR)-5,5-difluoro-5a-methyl-1,4,4a,5,5a,6-hexahydrocyclopropa[f]indazol-3-yl)-1H-indol-6-yl)-3-(1-(5-(2,6-dioxopiperidin-3-yl)pyridin-2-yl)piperidin-4-yl)-N-methylpropanamide (0.0237 g, 68.4%) was prepared by procedures analogous to Example 101 starting from tert-butyl 3-(piperidin-4-yl)propanoate (400 mg, 1.88 mmol) and 5-bromo-2-fluoropyridine (0.330 g, 1.88 mmol). ¹H NMR (500 MHz, DMSO-d₆) δ 12.70 (s, 1H), 11.43 (s, 1H), 10.89 (s, 1H), 7.84 (s, 1H), 7.68 (s, 1H), 7.58 (d, J=8.2 Hz, 1H), 7.26 (s, 1H), 7.13 (s, 1H), 6.90 (d, J=8.3 Hz, 1H), 6.64 (s, 1H), 4.10 (d, J=13.1 Hz, 2H), 3.84 (d, J=12.5 Hz, 1H), 3.20 (s, 3H), 3.18-2.99 (m, 3H), 2.98-2.80 (m, 3H), 2.75-2.46 (m, 2H), 2.27-2.20 (m, 1H), 2.08 (t, J=7.2 Hz, 2H), 1.98-1.92 (m, 1H), 1.87 (dd, J=15.4, 7.0 Hz, 1H), 1.57 (d, J=13.0 Hz, 2H), 1.49-1.41 (m, 3H), 1.40 (s, 3H), 1.04-0.98 (m, 2H). LCMS: C₃₆H₃₉F₂N₇O₃ requires: 655, found: m/z=656 [M+H]⁺.

### Example 167. 3-(6-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)pyridin-3-yl)piperidine-2,6-dione (22)

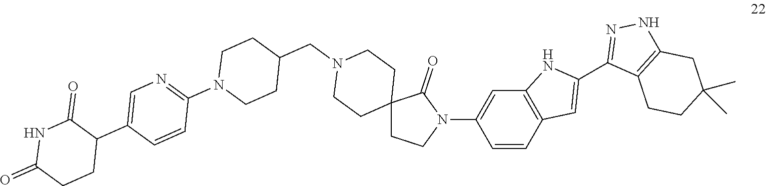 [SMILES: CC1(C)CCc2c(-c3cc4ccc(N5CCC6(CCN(CC7CCN(c8ccc(C9CCC(=O)NC9=O)cn8)CC7)CC6)C5=O)cc4[nH]3)n[nH]c2C1]

3-(6-(4-((2-(2-(6,6-dimethyl-4,5,6,7-tetrahydro-1H-indazol-3-yl)-1H-indol-6-yl)-1-oxo-2,8-diazaspiro[4.5]decan-8-yl)methyl)piperidin-1-yl)pyridin-3-yl)piperidine-2,6-dione (0.0221 g, 44.0%) was prepared by procedures analogous to Example 91 starting from 2-[2-(6,6-dimethyl-1,4,5,7-tetrahydroindazol-3-yl)-1H-indol-6-yl]-2,8-diazaspiro[4.5]decan-1-one (20.60 mg, 0.05 mmol) and 1-(5-(2,6-dioxopiperidin-3-yl)pyridin-2-yl)piperidine-4-carbaldehyde (21.1 mg, 0.07 mmol). ¹H NMR (500 MHz, DMSO-d₆) δ 12.51 (s, 1H), 11.28 (s, 1H), 10.83 (s, 1H), 8.94 (s, 1H), 7.97 (d, J=2.4 Hz, 1H), 7.74 (d, J=9.3 Hz, 1H), 7.50 (d, J=8.6 Hz, 1H), 7.42 (d, J=8.7 Hz, 1H), 7.25 (d, J=8.5 Hz, 1H), 6.87 (d, J=9.0 Hz, 1H), 6.56 (s, 1H), 4.31 (d, J=13.0 Hz, 2H), 3.93-3.85 (m, 2H), 3.75 (dd, J=12.1, 4.9 Hz, 1H), 3.60 (d, J=11.7 Hz, 2H), 3.48 (s, 1H), 3.13-3.04 (m, 4H), 2.86 (t, J=12.3 Hz, 2H), 2.75-2.64 (m, 3H), 2.42 (s, 2H), 2.25-2.08 (m, 5H), 2.03-1.95 (m, 1H), 1.87-1.80 (m, 4H), 1.59 (t, J=6.3 Hz, 2H), 1.35-1.16 (m, 3H), 1.02 (s, 6H). LCMS: C₄₁H₅₀N₈O₃ requires: 702, found: m/z=703 [M+H]⁺.

### Example 168. (2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(4-(2,6-dioxopiperidin-3-yl)pyridin-2-yl)piperidin-4-yl)methyl)piperazin-1-yl)-N-methylpropanamide (23)

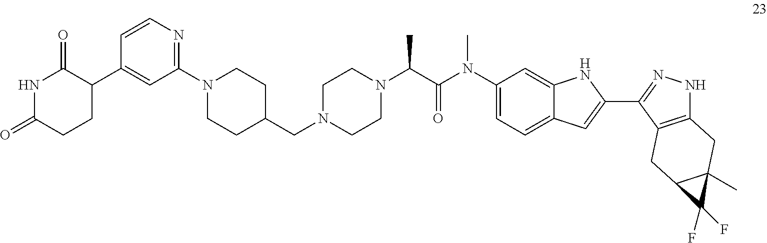 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3cc(C4CCC(=O)NC4=O)ccn3)CC2)CC1]

(2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(4-(2,6-dioxopiperidin-3-yl)pyridin-2-yl)piperidin-4-yl)methyl)piperazin-1-yl)-N-methylpropanamide (0.0307 g, 47.1%) was prepared by procedures analogous to Example 91 starting from 3-(2-(4-(hydroxymethyl)piperidin-1-yl)pyridin-4-yl)piperidine-2,6-dione (25.0 mg, 0.08 mmol) and (2S)—N-{2-[(4aS,5aR)-5,5-difluoro-5a-methyl-1H,4H,4aH,6H-cyclopropa[ ]indazol-3-yl]-1H-indol-6-yl}-N-methyl-2-(piperazin-1-yl)propanamide (38.9 mg, 0.08 mmol). ¹HNMR (500 MHz, DMSO-d₆) δ 12.72 (s, 1H), 11.45 (s, 1H), 10.89 (s, 1H), 8.97 (s, 1H), 8.03 (d, J=5.3 Hz, 1H), 7.60 (d, J=8.1 Hz, 1H), 7.31 (s, 1H), 6.96-6.92 (m, 1H), 6.78 (s, 1H), 6.65 (s, 1H), 6.55-6.51 (m, 1H), 4.29-4.25 (m, 2H), 3.82-3.75 (m, 1H), 3.63-3.57 (m, 1H), 3.23 (s, 3H), 3.17-2.73 (m, 14H), 2.72-2.56 (m, 2H), 2.32-2.12 (m, 1H), 2.10-1.95 (m, 2H), 1.92-1.84 (m, 1H), 1.77 (s, 3H), 1.42-1.38 (m, 4H), 1.30-1.00 (m, 5H). LCMS: C₄₁H₄₉F₂N₉O₃ requires: 753, found: m/z=754 [M+H]⁺.

### Example 169. 3-(6-(4-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decane-8-carbonyl)piperidin-1-yl)pyridin-3-yl)piperidine-2,6-dione (24)

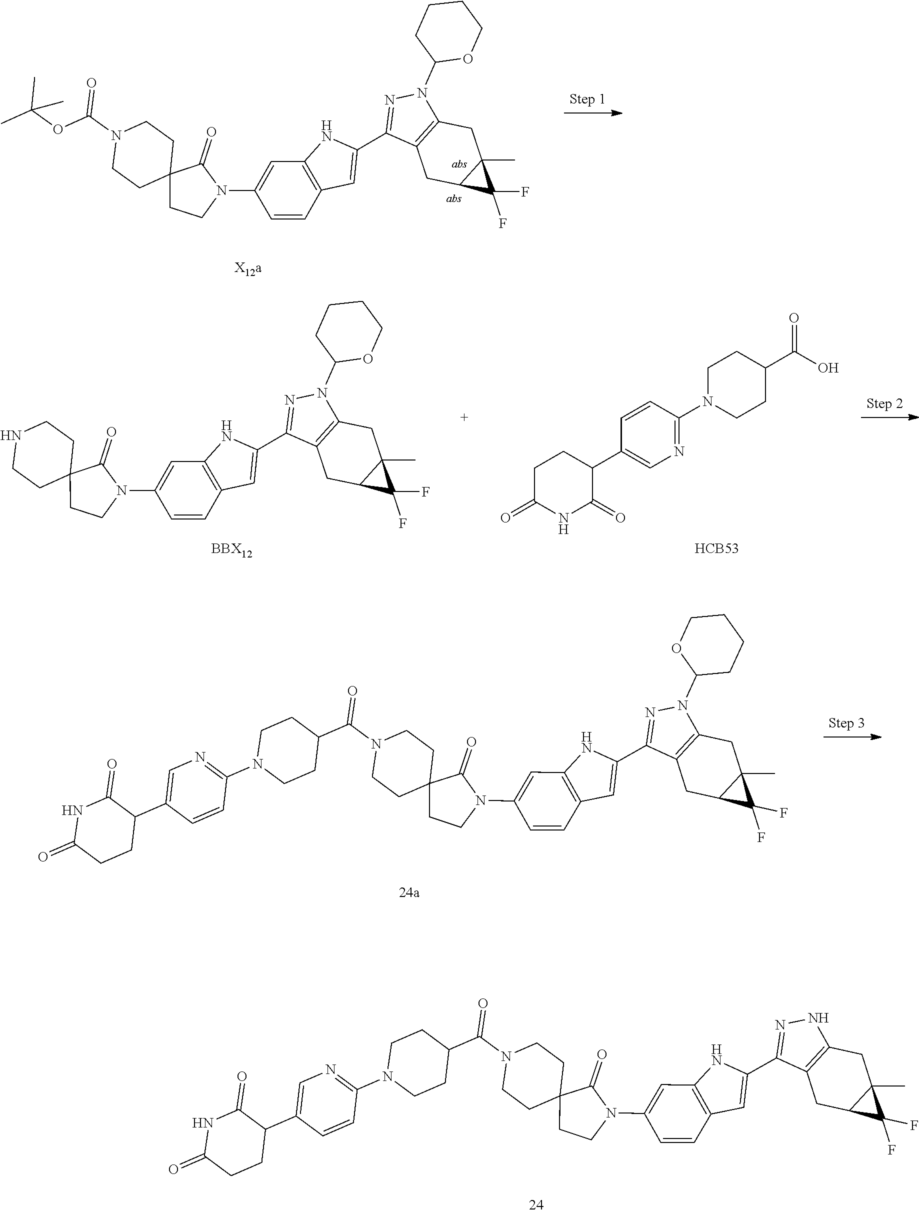 [SMILES: CC(C)(C)OC(=O)N1CCC2(CC1)CCN(c1ccc3cc(-c4nn(C5CCCCO5)c5c4C[C@@H]4C(F)(F)[C@]4(C)C5)[nH]c3c1)C2=O ; C[C@@]12Cc3c(c(-c4cc5ccc(N6CCC7(CCNCC7)C6=O)cc5[nH]4)nn3C3CCCCO3)C[C@@H]1C2(F)F ; C[C@@]12Cc3c(c(-c4cc5ccc(N6CCC7(CCN(C(=O)C8CCN(c9ccc(C%10CCC(=O)NC%10=O)cn9)CC8)CC7)C6=O)cc5[nH]4)nn3C3CCCCO3)C[C@@H]1C2(F)F ; O=C1CCC(c2ccc(N3CCC(C(=O)O)CC3)nc2)C(=O)N1 ; "HCB53" ; C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F]

### Step 1: 2-{2-[(4aS,5aR)-5,5-difluoro-5a-methyl-1-(oxan-2-yl)-4H,4aH,6H-cyclopropa[f]indazol-3-yl]-1H-indol-6-yl}-2,8-diazaspiro[4.5]decan-1-one (BBX₁₂)

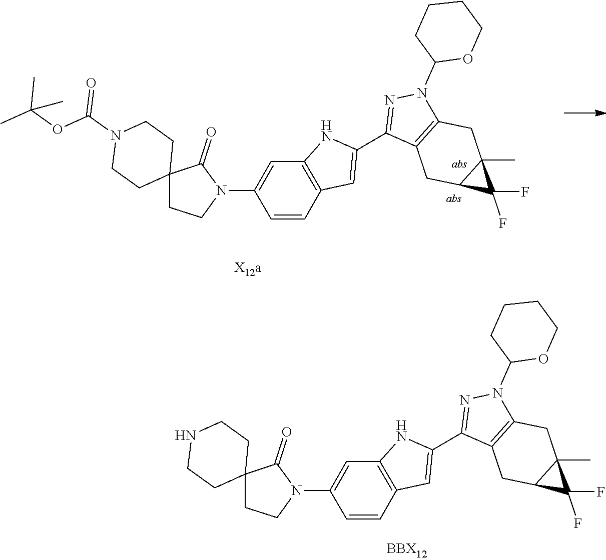 [SMILES: CC(C)(C)OC(=O)N1CCC2(CC1)CCN(c1ccc3cc(-c4nn(C5CCCCO5)c5c4C[C@@H]4C(F)(F)[C@]4(C)C5)[nH]c3c1)C2=O ; C[C@@]12Cc3c(c(-c4cc5ccc(N6CCC7(CCNCC7)C6=O)cc5[nH]4)nn3C3CCCCO3)C[C@@H]1C2(F)F]

tert-butyl 2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carboxylate (31.00 mg, 0.05 mmol) was stirred in DCM (1.50 mL) and TFA (0.50 mL) for ten minutes. The mixture was concentrated in vacuo to provide 2-{2-[(4aS,5aR)-5,5-difluoro-5a-methyl-1-(oxan-2-yl)-4H,4aH,6H-cyclopropa[f]indazol-3-yl)-1H-indol-6-yl}-2,8-diazaspiro[4.5]decan-1-one (26.8 mg, 100%). LCMS: C₃₀H₃₅F₂N₅O requires: 535, found: m/z=536 [M+H]⁺.

### Step 2: (3RS)-3-{6-[4-(2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carbonyl)piperidin-1-yl)pyridin-3-yl}piperidine-2,6-dione (25a)

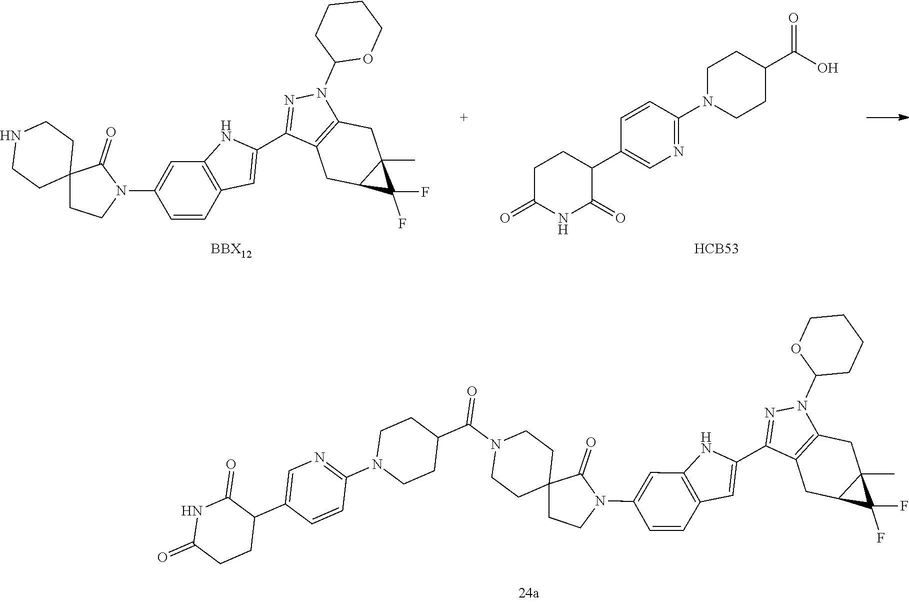 [SMILES: C[C@@]12Cc3c(c(-c4cc5ccc(N6CCC7(CCNCC7)C6=O)cc5[nH]4)nn3C3CCCCO3)C[C@@H]1C2(F)F ; C[C@@]12Cc3c(c(-c4cc5ccc(N6CCC7(CCN(C(=O)C8CCN(c9ccc(C%10CCC(=O)NC%10=O)cn9)CC8)CC7)C6=O)cc5[nH]4)nn3C3CCCCO3)C[C@@H]1C2(F)F ; O=C1CCC(c2ccc(N3CCC(C(=O)O)CC3)nc2)C(=O)N1 ; "HCB53"]

To a mixture of 2-{2-[(4aS,5aR)-5,5-difluoro-5a-methyl-1-(oxan-2-yl)-4H,4aH,6H-cyclopropa[f]indazol-3-yl)-1H-indol-6-yl}-2,8-diazaspiro[4.5]decan-1-one (26.8 mg, 0.05 mmol) in DCM (1.00 mL) was added N,N-diisopropylethylamine (0.05 mL, 0.04 g, 0.30 mmol) followed by 1-(5-(2,6-dioxopiperidin-3-yl)pyridin-2-yl)piperidine-4-carboxylic acid (15.8 mg, 0.05 mmol) then HATU (19.01 mg, 0.05 mmol) and DMF (1.00 mL). After stirring overnight, water was added and the mixture was extracted twice with 10% MeOH:DCM. The combined organic layers were concentrated and the crude residue was purified by preparative TLC eluted with 20% MeOH:DCM to provide (3RS)-3-{6-[4-(2-{2-[(4aS,5aR)-5,5-difluoro-5a-methyl-1-(oxan-2-yl)-4H,4aH,6H-cyclopropa[indazol-3-yl]-1H-indol-6-yl}-1-oxo-2,8-diazaspiro[4.5]decane-8-carbonyl)piperidin-1-yl]pyridin-3-yl}piperidine-2,6-dione (0.0145 g, 34.7%). LCMS: C₄₆H₅₂F₂N₈O₅ requires: 834, found: m/z=835 [M+H]⁺.

### Step 3: 3-(6-(4-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decane-8-carbonyl)piperidin-1-yl)pyridin-3-yl)piperidine-2,6-dione (24)

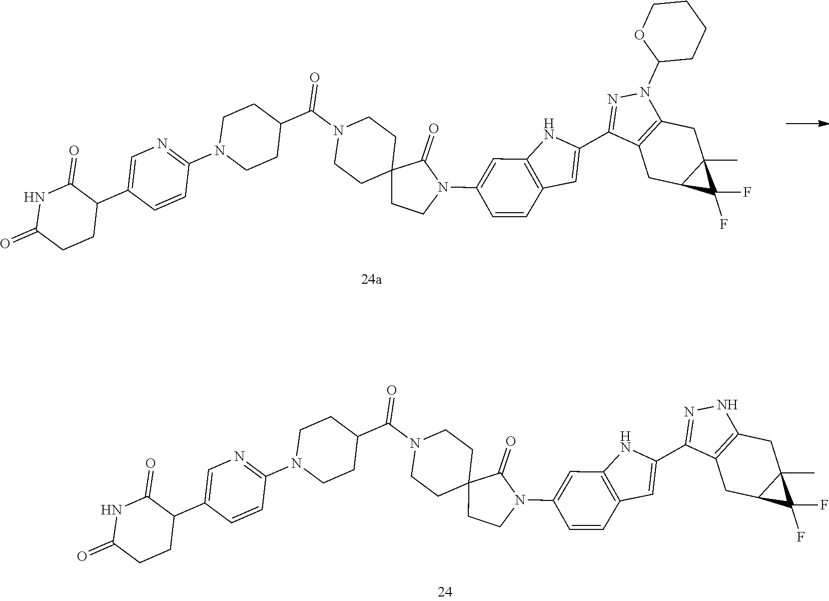 [SMILES: C[C@@]12Cc3c(c(-c4cc5ccc(N6CCC7(CCN(C(=O)C8CCN(c9ccc(C%10CCC(=O)NC%10=O)cn9)CC8)CC7)C6=O)cc5[nH]4)nn3C3CCCCO3)C[C@@H]1C2(F)F ; C[C@@]12Cc3[nH]nc(-c4cc5ccc(N6CCC7(CCN(C(=O)C8CCN(c9ccc(C%10CCC(=O)NC%10=O)cn9)CC8)CC7)C6=O)cc5[nH]4)c3C[C@@H]1C2(F)F]

A mixture of (3RS)-3-{6-[4-(2-{2-[(4aS,5aR)-5,5-difluoro-5a-methyl-1-(oxan-2-yl)-4H,4aH,6H-cyclopropa[f]indazol-3-yl]-1H-indol-6-yl}-1-oxo-2,8-diazaspiro[4.5]decane-8-carbonyl)piperidin-1-yl]pyridin-3-yl}piperidine-2,6-dione (14.5 mg, 0.02 mmol) and para-toluene sulfonate hydrate (6.6 mg, 0.03 mmol) in EtOH (2.00 mL) was heated in a 90° C. heating block for twenty minutes. The mixture was then cooled and dilute aq. sodium bicarbonate was added. The mixture was extracted with DCM then with 10% MeOH:DCM. The combined organic layers were concentrated and the crude residue was purified by preparative TLC eluted with 10% MeOH:DCM to provide 3-(6-(4-(2-(2-((4aS,5aR)-5,5-difluoro-5a-methyl-1,4,4a,5,5a,6-hexahydrocyclopropa[ ]indazol-3-yl)-1H-indol-6-yl)-1-oxo-2,8-diazaspiro[4.5]decane-8-carbonyl)piperidin-1-yl)pyridin-3-yl)piperidine-2,6-dione (0.0046 g, 35.3%). ¹HNMR (500 MHz, DMSO-d₆) δ 12.62 (s, 1H), 11.25 (s, 1H), 10.88 (s, 1H), 7.93 (d, J=2.4 Hz, 1H), 7.76 (s, 1H), 7.63 (s, 1H), 7.50 (d, J=8.5 Hz, 1H), 7.26 (d, J=8.6 Hz, 1H), 7.09 (s, 1H), 6.57 (s, 1H), 4.32-4.22 (m, 3H), 4.04 (d, J=13.7 Hz, 1H), 3.88 (t, J=6.9 Hz, 2H), 3.30 (t, J=12.4 Hz, 1H), 3.21-2.95 (m, 5H), 2.94-2.80 (m, 2H), 2.75-2.64 (m, 1H), 2.32-2.08 (m, 3H), 2.03-1.97 (m, 1H), 1.86 (dd, J=14.9, 7.1 Hz, 1H), 1.77-1.74 (m, 3H), 1.66-1.57 (m, 5H), 1.40 (s, 3H), 1.34-1.20 (m, 2H), 0.95-0.83 (m, 1H). LCMS: C₄₁H₄₄F₂N₈O₄ requires: 750, found: m/z=751 [M+H]⁺.

### Example 170. 3-(6-(4-(2-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)ethyl)piperidin-1-yl)pyridin-3-yl)piperidine-2,6-dione (25)

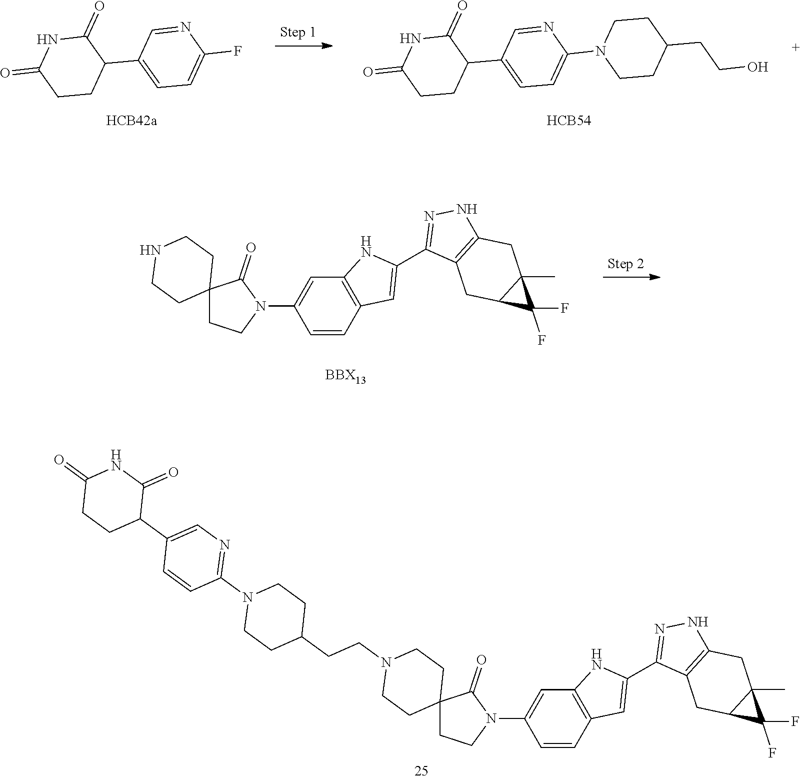

### Step 1: 3-(6-(4-(2-hydroxyethyl)piperidin-1-yl)pyridin-3-yl)piperidine-2,6-dione (HCB54)

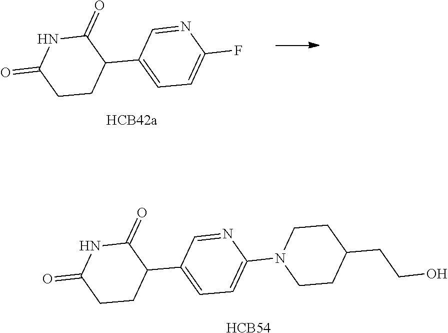

A mixture of 3-(6-fluoropyridin-3-yl)piperidine-2,6-dione (43.4 mg, 0.21 mmol), 4-piperidineethanol (26.9 mg, 0.21 mmol), and N,N-diisopropylethylamine (0.07 mL, 0.05 g, 0.42 mmol) in DMSO (1.00 mL) was heated in a 100° C. heating block for two days. Water was added and the mixture was extracted three times with ethyl acetate. The combined organic layers were concentrated and the crude residue was purified by preparative TLC in 5% MeOH:DCM to provide 3-(6-(4-(2-hydroxyethyl)piperidin-1-yl)pyridin-3-yl)piperidine-2,6-dione (0.0149 g, 22.5%). LCMS: C₁₇H₂₃N₃O₃ requires: 317, found: m/z=318 [M+H]⁺.

### Step 2: 3-(6-(4-(2-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)ethyl)piperidin-1-yl)pyridin-3-yl)piperidine-2,6-dione (25)

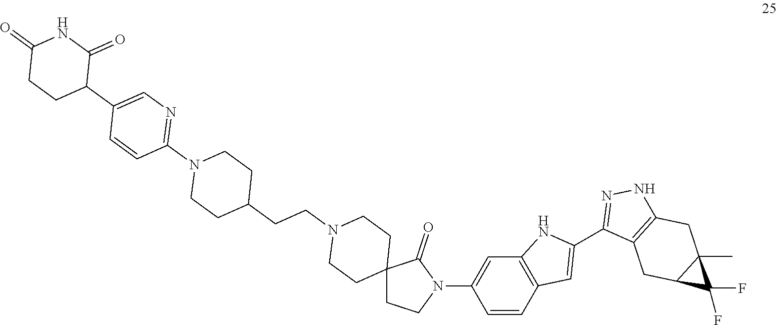 [SMILES: C[C@@]12Cc3[nH]nc(-c4cc5ccc(N6CCC7(CCN(CCC8CCN(c9ccc(C%10CCC(=O)NC%10=O)cn9)CC8)CC7)C6=O)cc5[nH]4)c3C[C@@H]1C2(F)F]

3-(6-(4-(2-(2-(2-((4aS,5aR)-5,5-difluoro-5a-methyl-1,4,4a,5,5a,6-hexahydrocyclopropa[f]indazol-3-yl)-1H-indol-6-yl)-1-oxo-2,8-diazaspiro[4.5]decan-8-yl)ethyl)piperidin-1-yl)pyridin-3-yl)piperidine-2,6-dione (0.0126 g, 93.7%) was prepared by procedures analogous to Example 91 starting from 3-(6-(4-(2-hydroxyethyl)piperidin-1-yl)pyridin-3-yl)piperidine-2,6-dione (14.9 mg, 0.05 mmol) and 2-{2-[(4aS,5aR)-5,5-difluoro-5a-methyl-1H,4H,4aH,6H-cyclopropa[indazol-3-yl]-1H-indol-6-yl}-2,8-diazaspiro[4.5]decan-1-one (22.6 mg, 0.05 mmol). ¹H NMR (500 MHz, DMSO-d₆) δ 12.64 (s, 1H), 11.31 (s, 1H), 10.82 (s, 1H), 9.13 (s, 1H), 7.95 (d, J=2.5 Hz, 1H), 7.74 (s, 1H), 7.51 (d, J=8.4 Hz, 1H), 7.41-7.35 (m, 1H), 7.25 (d, J=8.6 Hz, 1H), 6.82 (d, J=8.8 Hz, 1H), 6.58-6.52 (m, 1H), 4.29 (d, J=12.8 Hz, 2H), 3.88 (s, 2H), 3.74 (dd, J=12.3, 4.9 Hz, 1H), 3.60-3.56 (m, 1H), 3.30 (s, 2H), 3.23-2.93 (m, 4H), 2.90-2.61 (m, 4H), 2.20-2.16 (m, 2H), 2.10-1.93 (m, 2H), 1.87-1.83 (m, 3H), 1.80-1.72 (m, 2H), 1.70-1.52 (m, 4H), 1.40 (s, 3H), 1.33-1.10 (m, 4H). LCMS: C₄₂H₄₈F₂N₈O₃ requires: 750, found: m/z=751 [M+H]⁺.

### Example 171. 3-{5-[4-({4-[2-(6,6-dimethyl-1,4,5,7-tetrahydroindazol-3-yl)-1H-indole-6-carbonyl]piperazin-1-yl}methyl)piperidin-1-yl]-1-oxo-3H-isoindol-2-yl}piperidine-2,6-dione (26)

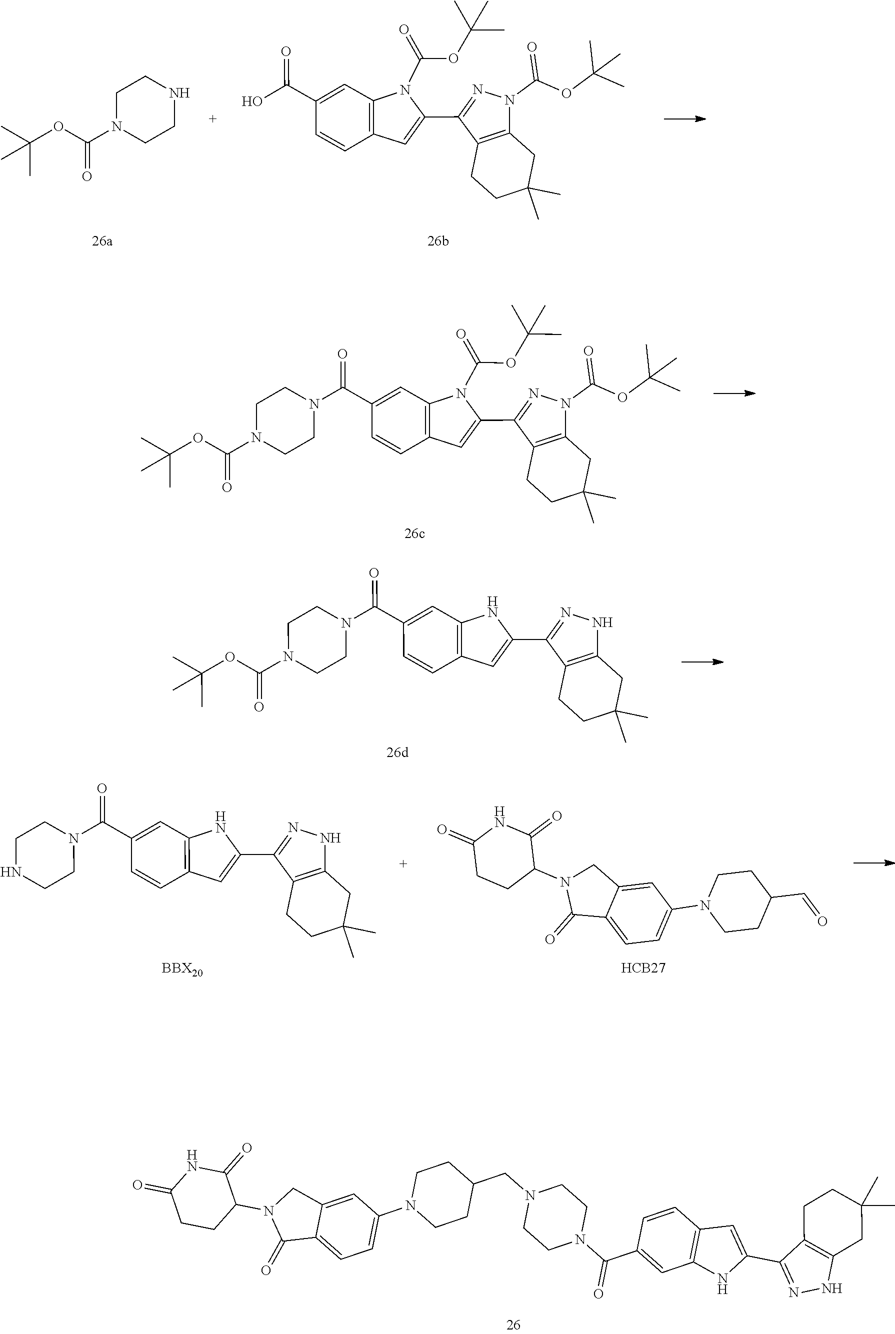

Step 1: 1-(tert-butoxycarbonyl)-2-[1-(tert-butoxycarbonyl)-6,6-dimethyl-5,7-dihydro-4H-indazol-3-yl]indole-6-carboxylic acid (46.00 mg, 0.09 mmol) and [(dimethylamino)({[1,2,3]triazolo[4,5-b]pyridin-3-yloxy})methylidene]dimethylazanium; hexafluoro-lambda5-phosphanuide (34.32 mg, 0.09 mmol) were dissolved in DMF (0.5 mL), and then N,N-diisopropylethylamine (63.06 uL, 46.67 mg, 0.36 mmol) was added. After the mixture was stirred for five minutes, tert-butyl piperazine-1-carboxylate (16.81 mg, 0.09 mmol) in DMF (0.25 mL) was added, and the reaction was stirred at room temperature for thirty minutes. The reaction mixture was dissolved in EtOAc (30 mL) and washed with water twice. The organic layer was dried over Na₂SO₄, filtered, and concentrated to afford tert-butyl 3-[1-(tert-butoxycarbonyl)-6-[4-(tert-butoxycarbonyl)piperazine-1-carbonyl]indol-2-yl]-6,6-dimethyl-5,7-dihydro-4H-indazole-1-carboxylate (61 mg, without purification). LCMS: C₃₇H₅₁N₅O₇ requires: 677.4, found: m/z=678.8 [M+H]⁺.

Step 2: To a solution of tert-butyl 3-[1-(tert-butoxycarbonyl)-6-[4-(tert-butoxycarbonyl)piperazine-1-carbonyl]indol-2-yl]-6,6-dimethyl-5,7-dihydro-4H-indazole-1-carboxylate (61 mg, 0.09 mmol) in EtOH (3 mL), was added 1 M NaOH (2.00 mL, 2.00 mmol) and the solution was heated at 90° C. for one hour. LCMS indicated completion of reaction. The reaction was cooled in an ice bath and 1 N HCl was added to adjust the pH to 4-5. Upon removal of EtOH a white solid precipitated. The aqueous layer was extracted with EtOAc (30 mL×2) and the organic layer was dried over Na₂SO₄, filtered, and concentrated under reduced pressure to afford tert-butyl 4-[2-(6,6-dimethyl-1,4,5,7-tetrahydroindazol-3-yl)-1H-indole-6-carbonyl]piperazine-1-carboxylate (39 mg, 91%). LCMS: C₂₇H₃₅N₅O₃ requires: 477.3, found: m/z=478.6 [M+H]⁺.

Step 3: tert-butyl 4-[2-(6,6-dimethyl-1,4,5,7-tetrahydroindazol-3-yl)-1H-indole-6-carbonyl]piperazine-1-carboxylate (39 mg, 0.08 mmol) was dissolved in DCM (4 mL), and trifluoroacetic acid (2 mL) was added. The reaction was stirred for thirty minutes and was then concentrated under reduced pressure to afford 6,6-dimethyl-3-[6-(piperazine-1-carbonyl)-1H-indol-2-yl]-1,4,5,7-tetrahydroindazole (30 mg, 97%). LCMS: C₂₂H₂₇N₅O requires: 377.2, found: m/z=378.5 [M+H]⁺.

Step 4: To a solution of 1-(2-(2,6-dioxopiperidin-3-yl)-1-oxoisoindolin-5-yl)piperidine-4-carbaldehyde (18.40 mg, 0.05 mmol) in DMSO (0.4 mL) was added 6,6-dimethyl-3-[6-(piperazine-1-carbonyl)-1H-indol-2-yl]-1,4,5,7-tetrahydroindazole trifluoroacetic acid salt (25.45 mg, 0.05 mmol) and triethylamine (10.5 mg, 0.1 mmol) and the reaction was stirred for 5 min. Then sodium triacetoxyborohydride (109.73 mg, 0.52 mmol, powder) was added, and the reaction was stirred for twenty minutes. LCMS indicated completion of the reaction. Water (0.5 mL) was added to the reaction mixtureand then the reaction mixture was purified by Prep-HPLC eluting with acetonitrile with 0.01% TFA and water with 0.01% TFA (5% to 95% gradient elution) to provide 3-{5-[4-({4-[2-(6,6-dimethyl-1,4,5,7-tetrahydroindazol-3-yl)-1H-indole-6-carbonyl]piperazin-1-yl}methyl)piperidin-1-yl]-1-oxo-3H-isoindol-2-yl}piperidine-2,6-dione (11.4 mg, 29%). LCMS: C₄₁H₄₈N₈O₄ requires: 716.4, found: m/z=717.8 [M+H]⁺. ¹H NMR (500 MHz, DMSO-d₆) δ 11.54 (s, 1H), 10.95 (s, 1H), 9.53 (s, 1H), 7.58 (d, J=8.1 Hz, 1H), 7.54 (d, J=9.4 Hz, 2H), 7.21 (s, 1H), 7.09 (t, J=7.5 Hz, 3H), 7.01 (s, 1H), 6.66 (s, 1H), 5.06 (dd, J=13.3, 5.1 Hz, 1H), 4.33 (d, J=16.8 Hz, 1H), 4.21 (d, J=16.8 Hz, 1H), 4.04 (s, 1H), 3.93 (d, J=12.7 Hz, 2H), 3.13 (d, J=13.2 Hz, 4H), 2.97-2.82 (m, 3H), 2.69 (t, J=6.4 Hz, 2H), 2.62 (t, J=16.2 Hz, 1H), 2.38 (td, J=14.4, 13.8, 9.5 Hz, 1H), 2.09 (d, J=11.8 Hz, 1H), 1.97 (dt, J=11.6, 5.9 Hz, 1H), 1.84 (d, J=12.5 Hz, 2H), 1.59 (t, J=6.4 Hz, 2H), 1.48 (t, J=6.8 Hz, 1H), 1.33 (d, J=12.2 Hz, 1H), 1.25 (s, 4H), 1.18 (t, J=7.0 Hz, 2H), 1.02 (s, 6H), 0.86 (t, J=6.7 Hz, 1H).

### Example 172. (4aS,5aR)-N-(1-((1S)-(1-(3-(2-((2-(2,6-dioxopiperidin-3-yl)-1,3-dioxoisoindolin-4-yl)amino)ethoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (27)

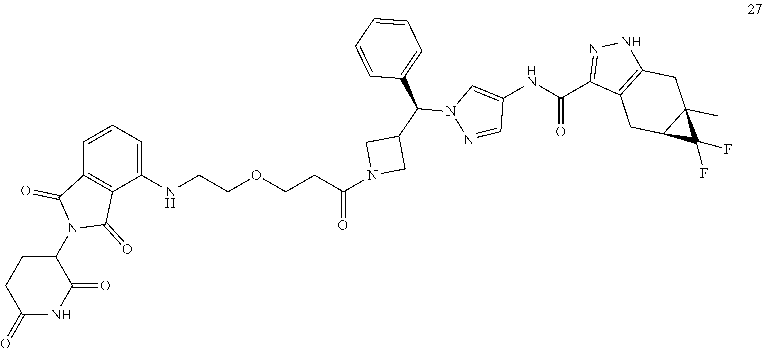 [SMILES: C[C@@]12Cc3[nH]nc(C(=O)Nc4cnn([C@H](c5ccccc5)C5CN(C(=O)CCOCCNc6cccc7c6C(=O)N(C6CCC(=O)NC6=O)C7=O)C5)c4)c3C[C@@H]1C2(F)F]

Using general procedure 1, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (15 mg, 0.034 mmol) was treated with 3-(2-((2-(2,6-dioxopiperidin-3-yl)-1,3-dioxoisoindolin-4-yl)amino)ethoxy)propanoic acid (20 mg, 0.051 mmol) to afford the desired product (14 mg, 0.018 mmol, 51%) ¹H NMR (500 MHz, (CD₃)₂SO) δ 12.95 (s, 1H), 11.09 (d, J=4.0 Hz, 1H), 10.17 (d, J=1.7 Hz, 1H), 8.05 (d, J=2.9 Hz, 1H), 7.64 (s, 1H), 7.57 (dd, J=8.6, 7.0 Hz, 1H), 7.38-7.27 (m, 5H), 7.13 (d, J=8.4 Hz, 1H), 7.03 (dd, J=7.1, 2.8 Hz, 1H), 6.58 (q, J=5.7 Hz, 1H), 5.63 (dd, J=10.9, 3.9 Hz, 1H), 5.04 (dt, J=11.1, 5.1 Hz, 1H), 4.13 (dt, J=44.1, 8.4 Hz, 1H), 3.85 (t, J=8.0 Hz, 1H), 3.78 (q, J=6.5, 4.0 Hz, 1H), 3.62 (td, J=6.4, 3.0 Hz, 2H), 3.60-3.48 (m, 3H), 3.46 (d, J=4.0 Hz, 2H), 3.09-2.98 (m, 3H), 2.92-2.77 (m, 1H), 2.27 (qd, J=16.2, 15.7, 6.7 Hz, 2H), 1.99 (td, J=9.0, 8.3, 4.9 Hz, 1H), 1.76 (dd, J=15.3, 6.2 Hz, 1H), 1.34 (s, 3H). LCMS: C₄₁H₄₁F₂N₉O₇ requires: 810, found: m/z=810 [M+H]⁺.

### Example 173. (4aS,5aR)-N-(1-((1S)-(1-(3-(2-(2-(2-((2-(2,6-dioxopiperidin-3-yl)-1,3-dioxoisoindolin-4-yl)amino)ethoxy)ethoxy)ethoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (28)

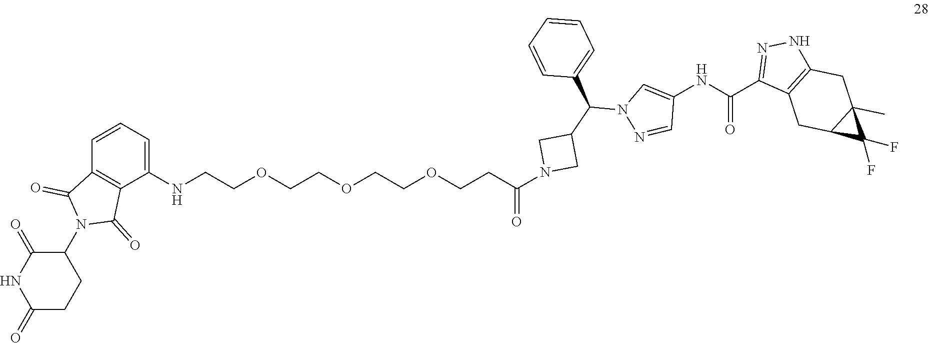 [SMILES: C[C@@]12Cc3[nH]nc(C(=O)Nc4cnn([C@H](c5ccccc5)C5CN(C(=O)CCOCCOCCOCCNc6cccc7c6C(=O)N(C6CCC(=O)NC6=O)C7=O)C5)c4)c3C[C@@H]1C2(F)F]

Using general procedure 1, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (15 mg, 0.034 mmol) was treated with 3-(2-(2-(2-((2-(2,6-dioxopiperidin-3-yl)-1,3-dioxoisoindolin-4-yl)amino)ethoxy)ethoxy)ethoxy)propanoic acid (16 mg, 0.034 mmol) to afford the desired product (8.1 mg, 0.0090 mmol, 26%)¹H NMR (500 MHz, CD₃OD) δ 8.03 (dt, J=5.1, 2.7 Hz, 1H), 7.66-7.63 (m, 1H), 7.49 (dddd, J=8.5, 7.3, 6.0, 2.0 Hz, 1H), 7.40-7.26 (m, 6H), 7.06-6.98 (m, 2H), 5.54 (dtd, J=11.3, 5.4, 3.2 Hz, 1H), 5.02 (dddd, J=9.7, 5.5, 4.1, 2.1 Hz, 1H), 4.88 (s, 4H), 4.80 (s, 1H), 4.34 (t, J=8.3 Hz, 1H), 4.24 (t, J=8.6 Hz, OH), 4.10-3.99 (m, 1H), 3.95 (td, J=9.8, 8.3, 3.7 Hz, 1H), 3.79-3.51 (m, 18H), 3.44 (dt, J=8.2, 5.3 Hz, 2H), 3.17-3.01 (m, 4H), 2.87-2.61 (m, 4H), 2.31 (tt, J=11.1, 4.1 Hz, 2H), 2.11-2.01 (m, 1H), 1.63 (dd, J=14.9, 6.8 Hz, 1H), 1.37 (s, 3H), 1.28 (s, 6H). LCMS: C₄₅H₄₉F₂N₉O₉ requires: 898, found: m/z=898 [M+H]⁺.

### Example 174. (4aS,5aR)-N-(1-((1S)-(1-(1-((2-(2,6-dioxopiperidin-3-yl)-1,3-dioxoisoindolin-4-yl)amino)-3,6,9,12,15-pentaoxaoctadecan-18-oyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (29)

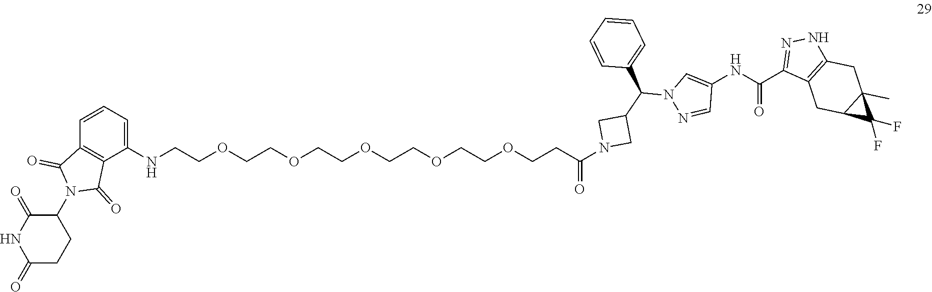 [SMILES: C[C@@]12Cc3[nH]nc(C(=O)Nc4cnn([C@H](c5ccccc5)C5CN(C(=O)CCOCCOCCOCCOCCOCCNc6cccc7c6C(=O)N(C6CCC(=O)NC6=O)C7=O)C5)c4)c3C[C@@H]1C2(F)F]

Using general procedure 1, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (15 mg, 0.034 mmol) was treated with 1-((2-(2,6-dioxopiperidin-3-yl)-1,3-dioxoisoindolin-4-yl)amino)-3,6,9,12,15-pentaoxaoctadecan-18-oic acid (16 mg, 0.034 mmol) to afford the desired product (8.1 mg, 0.0090 mmol, 26%). ¹H NMR (500 MHz, CD₃OD) δ 8.04 (d, J=5.6 Hz, 1H), 7.66 (d, J=2.4 Hz, 1H), 7.54-7.40 (m, 1H), 7.40-7.28 (m, 4H), 7.08-6.98 (m, 2H), 5.56 (dd, J=10.8, 3.7 Hz, 1H), 5.03 (dd, J=12.6, 5.4 Hz, 1H), 4.85-4.81 (m, 1H), 4.39-4.26 (m, 1H), 4.12-4.01 (m, 1H), 3.96 (d, J=7.2 Hz, 1H), 3.80-3.64 (m, 4H), 3.64-3.51 (m, 15H), 3.46 (dt, J=5.5, 2.7 Hz, 1H), 3.18-3.02 (m, 3H), 2.87-2.63 (m, 3H), 2.40-2.29 (m, 2H), 2.11-2.05 (m, 1H), 1.64 (dd, J=14.6, 6.6 Hz, 1H), 1.37 (s, 3H). LCMS: C₄₉H₅₇F₂N₉O₁₁ requires: 986, found: m/z=987 [M+H]⁺.

### Example 175. (4aS,5aR)-N-(1-((1S)-(1-(1-((2-(2,6-dioxopiperidin-3-yl)-1,3-dioxoisoindolin-4-yl)amino)-3,6,9,12,15,18,21,24-octaoxaheptacosan-27-oyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (30)

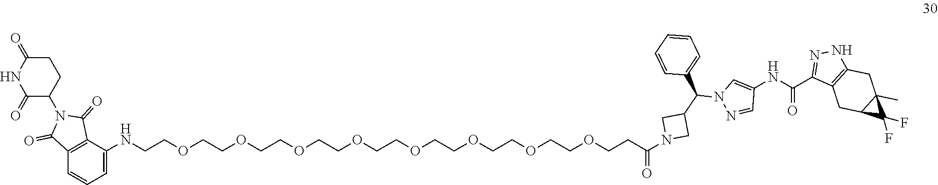 [SMILES: C[C@@]12Cc3[nH]nc(C(=O)Nc4cnn([C@H](c5ccccc5)C5CN(C(=O)CCOCCOCCOCCOCCOCCOCCOCCOCCNc6cccc7c6C(=O)N(C6CCC(=O)NC6=O)C7=O)C5)c4)c3C[C@@H]1C2(F)F]

using general procedure 1, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (15 mg, 0.034 mmol) was treated with 1-((2-(2,6-dioxopiperidin-3-yl)-1,3-dioxoisoindolin-4-yl)amino)-3,6,9,12,15,18,21,24-octaoxaheptacosan-27-oic acid (24 mg, 0.034 mmol) to afford the desired product (2.6 mg, 0.0023 mmol, 6.8%). ¹H NMR (500 MHz, CD₃OD) δ 8.04 (d, J=7.3 Hz, 1H), 7.67 (d, J=1.8 Hz, 1H), 7.53 (ddd, J=8.7, 7.1, 1.7 Hz, 1H), 7.39-7.29 (m, 4H), 7.10-7.01 (m, 2H), 5.56 (dd, J=10.8, 3.8 Hz, 1H), 5.03 (dd, J=12.5, 5.5 Hz, 1H), 4.88 (s, 1H), 4.80 (s, 1H), 4.32 (dt, J=49.2, 8.6 Hz, 1H), 4.12-4.02 (m, 1H), 3.98 (s, 1H), 3.80-3.66 (m, 4H), 3.64-3.51 (m, 25H), 3.47 (td, J=5.2, 2.8 Hz, 2H), 3.34 (p, J=1.7 Hz, 1H), 3.26 (p, J=1.6 Hz, 1H), 3.10 (dt, J=38.8, 17.4 Hz, 3H), 2.89-2.64 (m, 3H), 2.34 (dtd, J=8.6, 6.1, 3.4 Hz, 2H), 2.08 (ddd, J=13.0, 5.4, 2.8 Hz, 1H), 1.68-1.55 (m, 2H), 1.37 (d, J=2.1 Hz, 3H), 1.28 (s, 9H), 0.88 (q, J=9.7, 8.2 Hz, 3H). LCMS: C₅₅H₆₉F₂N₉O₁₄ requires: 1118, found: m/z=1119 [M+H]⁺.

### Example 176. (4aS,5aR)-N-(1-((1S)-(1-(17-((2-(2,6-dioxopiperidin-3-yl)-1,3-dioxoisoindolin-4-yl)amino)-3,6,9,12,15-pentaoxaheptadecyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (31)

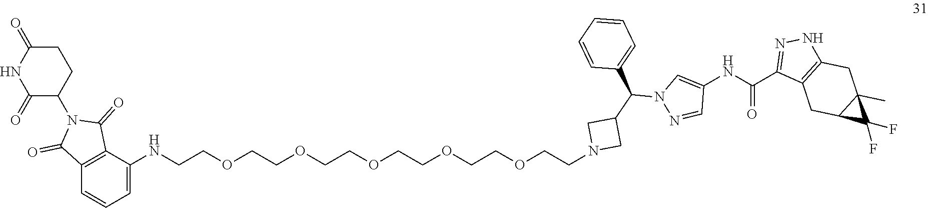 [SMILES: C[C@@]12Cc3[nH]nc(C(=O)Nc4cnn([C@H](c5ccccc5)C5CN(CCOCCOCCOCCOCCOCCNc6cccc7c6C(=O)N(C6CCC(=O)NC6=O)C7=O)C5)c4)c3C[C@@H]1C2(F)F]

Using general procedure 2, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (10 mg, 0.023 mmol) was treated with 17-((2-(2,6-dioxopiperidin-3-yl)-1,3-dioxoisoindolin-4-yl)amino)-3,6,9,12,15-pentaoxaheptadecyl 4-methylbenzenesulfonate (16 mg, 0.023 mmol) to afford the desired product (2.5 mg, 0.0026 mmol, 11%). ¹H NMR (500 MHz, CD₃OD) δ 8.02 (s, 1H), 7.65 (s, 1H), 7.52 (dd, J=8.6, 7.1 Hz, 1H), 7.37-7.26 (m, 5H), 7.05 (dd, J=13.8, 7.8 Hz, 2H), 5.55 (d, J=10.4 Hz, 1H), 5.03 (dd, J=12.5, 5.5 Hz, 1H), 4.88 (s, 1H), 4.80 (s, 1H), 3.69 (t, J=5.3 Hz, 2H), 3.62 (q, J=1.4 Hz, 4H), 3.60-3.51 (m, 9H), 3.48 (dt, J=10.6, 5.2 Hz, 3H), 3.34 (s, 3H), 3.26 (p, J=1.7 Hz, 1H), 3.18-3.01 (m, 4H), 2.87-2.64 (m, 6H), 2.12 -2.04 (m, 2H), 1.64 (dd, J=15.2, 7.2 Hz, 2H), 1.37 (d, J=2.2 Hz, 3H), 1.28 (s, 1H). LCMS: C₄₅H₅₇F₂N₉O₁₀ requires: 958, found: m/z=959 [M+H]⁺.

### Example 177. (4aS,5aR)-N-(1-((1S)-(1-(5-(2-(2,6-dioxopiperidin-3-yl)-1-oxoisoindolin-4-yl)pentanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (32)

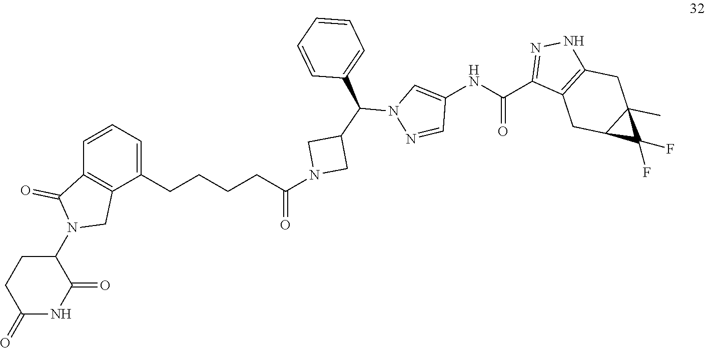 [SMILES: C[C@@]12Cc3[nH]nc(C(=O)Nc4cnn([C@H](c5ccccc5)C5CN(C(=O)CCCCc6cccc7c6CN(C6CCC(=O)NC6=O)C7=O)C5)c4)c3C[C@@H]1C2(F)F]

Using general procedure 1, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (20 mg, 0.050 mmol) was treated with 5-(2-(2,6-dioxopiperidin-3-yl)-1-oxoisoindolin-4-yl)pentanoic acid (16 mg, 0.050 mmol) to afford the desired product (19 mg, 0.02 mmol, 54%). ¹H NMR (500 MHz, (CD₃)₂SO) δ 12.95 (s, 1H), 10.98 (s, 1H), 10.17 (d, J=4.5 Hz, 1H), 8.06 (d, J=1.8 Hz, 1H), 7.65 (s, 1H), 7.56 (td, J=4.1, 2.0 Hz, 1H), 7.46-7.43 (m, 2H), 7.40-7.31 (m, 4H), 7.34-7.27 (m, 1H), 5.68-5.63 (m, 1H), 5.13 (d, J=13.4 Hz, 1H), 4.45 (d, J=17.2 Hz, 1H), 4.29 (d, J=17.1 Hz, 1H), 4.12 (dt, J=41.1, 8.2 Hz, 1H), 3.88-3.81 (m, 1H), 3.76 (dt, J=16.7, 8.5 Hz, 1H), 3.53 (ddd, J=25.5, 9.4, 5.7 Hz, 1H), 3.04 (s, 2H), 3.00 (s, 1H), 2.96-2.87 (m, 1H), 2.81 (d, J=17.5 Hz, 1H), 2.66-2.60 (m, 3H), 2.58 (s, 1H), 2.41 (td, J=13.2, 4.4 Hz, 1H), 2.04 (ddt, J=16.0, 10.7, 5.6 Hz, 4H), 1.80-1.74 (m, 1H), 1.58 (s, 2H), 1.53-1.46 (m, 2H), 1.34 (s, 3H). LCMS: C₄₁H₄₂F₂N₈O₅ requires: 765, found: m/z=788 [M+Na]⁺.

### Example 178. (2S)—N-(2-(6,6-dimethyl-4,5,6,7-tetrahydro-1H-indazol-3-yl)-1H-indol-6-yl)-2-(4-(((3R)-1-(2-(2,6-dioxopiperidin-3-yl)-1,3-dioxoisoindolin-5-yl)pyrrolidin-3-yl)methyl)piperazin-1-yl)-N-methylpropanamide (33

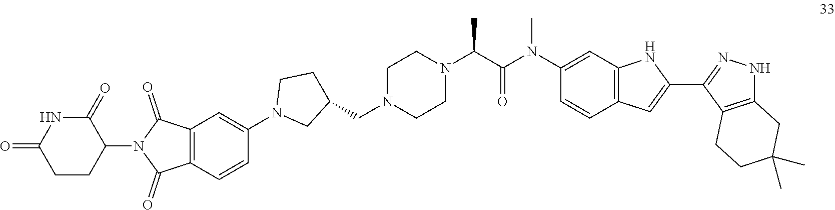 [SMILES: C[C@@H](C(=O)N(C)c1ccc2cc(-c3n[nH]c4c3CCC(C)(C)C4)[nH]c2c1)N1CCN(C[C@H]2CCN(c3ccc4c(c3)C(=O)N(C3CCC(=O)NC3=O)C4=O)C2)CC1]

Using general procedure 4, (S)—N-(2-(6,6-dimethyl-4,5,6,7-tetrahydro-1H-indazol-3-yl)-1H-indol-6-yl)-N-methyl-2-(piperazin-1-yl)propanamide (15 mg, 0.030 mmol) was treated with (3S)-1-(2-(2,6-dioxopiperidin-3-yl)-1,3-dioxoisoindolin-5-yl)pyrrolidine-3-carbaldehyde (12 mg, 0.030 mmol) to afford the desired product (14 mg, 0.02 mmol, 53%). ¹H NMR (500 MHz, CD₃CN) δ 9.86 (s, 1H), 8.90 (s, 1H), 7.59 (dd, J=8.4, 6.4 Hz, 2H), 7.35 (s, 1H), 6.94 (d, J=8.3 Hz, 1H), 6.90 (d, J=2.2 Hz, 1H), 6.75 (dd, J=8.5, 2.3 Hz, 1H), 6.68 (d, J=2.1 Hz, 1H), 4.93 (dd, J=12.1, 5.4 Hz, 1H), 3.51 (dt, J=33.2, 10.6 Hz, 2H), 3.38 (q, J=8.8, 8.4 Hz, 1H), 3.34-3.29 (m, 1H), 3.24 (s, 3H), 3.15 (t, J=8.7 Hz, 1H), 2.72 (dddd, J=21.8, 18.0, 13.2, 4.6 Hz, 4H), 2.63 (s, 2H), 2.45 (s, 3H), 1.76 (dt, J=15.7, 8.0 Hz, 1H), 1.63 (t, J=6.4 Hz, 2H), 1.09 (d, J=6.7 Hz, 3H), 1.04 (s, 6H). LCMS: C₄₃H₅₁N₉O₅ requires: 774, found: m/z=775 [M+H]⁺.

### Example 179. (2S)—N-(2-(6,6-dimethyl-4,5,6,7-tetrahydro-1H-indazol-3-yl)-1H-indol-6-yl)-2-(4-(1-(1-(2-(2,6-dioxopiperidin-3-yl)-1,3-dioxoisoindolin-5-yl)piperidin-4-yl)azetidine-3-carbonyl)piperazin-1-yl)-N-methylpropanamide (34)

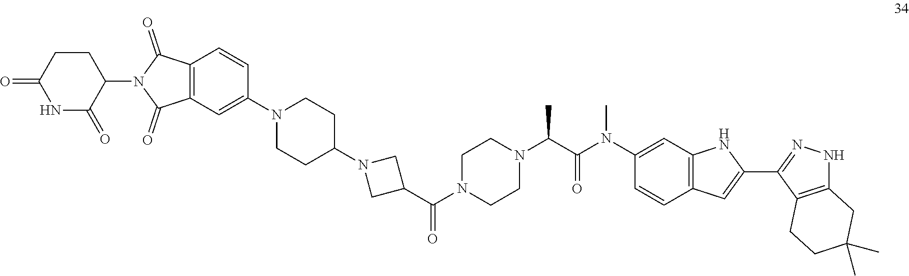 [SMILES: C[C@@H](C(=O)N(C)c1ccc2cc(-c3n[nH]c4c3CCC(C)(C)C4)[nH]c2c1)N1CCN(C(=O)C2CN(C3CCN(c4ccc5c(c4)C(=O)N(C4CCC(=O)NC4=O)C5=O)CC3)C2)CC1]

Using general procedure 5, (S)—N-(2-(6,6-dimethyl-4,5,6,7-tetrahydro-1H-indazol-3-yl)-1H-indol-6-yl)-N-methyl-2-(piperazin-1-yl)propanamide (20 mg, 0.050 mmol) was treated with 1-(1-(2-(2,6-dioxopiperidin-3-yl)-1,3-dioxoisoindolin-5-yl)piperidin-4-yl)azetidine-3-carboxylic acid (20 mg, 0.050 mmol) to afford the desired product (9.4 mg, 0.01 mmol, 24%). ¹H NMR (500 MHz, (CD₃)₂SO) δ 12.55 (s, 1H), 11.39 (s, 1H), 11.07 (s, 1H), 7.64 (d, J=8.5 Hz, 1H), 7.54 (d, J=8.3 Hz, 1H), 7.30 (s, 2H), 7.22 (d, J=8.6 Hz, 1H), 6.89 (d, J=8.1 Hz, 1H), 6.60 (s, 1H), 5.06 (dd, J=12.6, 5.4 Hz, 1H), 3.80 (s, 2H), 3.38 (s, 5H), 3.25 (d, J=6.8 Hz, 1H), 3.18 (s, 4H), 3.10 (s, 3H), 2.93-2.82 (m, 1H), 2.72-2.53 (m, 4H), 2.41 (s, 3H), 2.21 (s, 3H), 2.05-1.97 (m, 1H), 1.69 (s, 2H), 1.58 (t, J=6.3 Hz, 2H), 1.22 (d, J=15.8 Hz, 2H), 1.01 (d, J=7.7 Hz, 9H). LCMS: C₄₇H₅₆N₁₀O₆ requires: 857, found: m/z=858 [M+H]⁺.

### Example 180. N-(2-(6,6-dimethyl-4,5,6,7-tetrahydro-1H-indazol-3-yl)-1H-indol-6-yl)-1-(1-(2-(2,6-dioxopiperidin-3-yl)-1,3-dioxoisoindolin-5-yl)piperidin-4-yl)-N-methylazetidine-3-carboxamide (35)

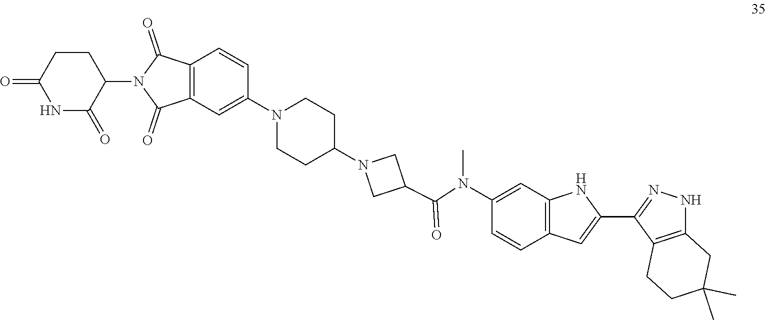 [SMILES: CN(C(=O)C1CN(C2CCN(c3ccc4c(c3)C(=O)N(C3CCC(=O)NC3=O)C4=O)CC2)C1)c1ccc2cc(-c3n[nH]c4c3CCC(C)(C)C4)[nH]c2c1]

Using general procedure 6, 2-(6,6-dimethyl-4,5,6,7-tetrahydro-1H-indazol-3-yl)-N-methyl-1H-indol-6-amine (20 mg, 0.070 mmol) was treated with 1-(1-(2-(2,6-dioxopiperidin-3-yl)-1,3-dioxoisoindolin-5-yl)piperidin-4-yl)azetidine-3-carboxylic acid (30 mg, 0.070 mmol) to afford the desired product (9.4 mg, 0.01 mmol, 17%). ¹H NMR (500 MHz, CD₃CN) δ 9.81 (s, 1H), 8.87 (s, 1H), 7.58 (d, J=8.4 Hz, 2H), 7.27-7.20 (m, 2H), 7.08 (dd, J=8.5, 2.3 Hz, 1H), 6.87 (dd, J=8.3, 1.9 Hz, 1H), 6.68 (d, J=2.1 Hz, 1H), 4.91 (dd, J=12.3, 5.4 Hz, 1H), 3.70 (d, J=13.0 Hz, 2H), 3.24 (s, 3H), 3.16 (d, J=24.6 Hz, 5H), 3.06 (d, J=9.2 Hz, 3H), 2.71 (tdd, J=22.8, 12.8, 8.3 Hz, 5H), 2.45 (s, 2H), 1.70-1.60 (m, 4H), 1.21 (d, J=10.5 Hz, 2H), 1.04 (s, 5H). LCMS: C₄₀H₄₄N₈O₅ requires: 717, found: m/z=718 [M+H]⁺.

### Example 181. (4aS,5aR)-N-(1-((1S)-(1-(1-(1-(5-((2,6-dioxopiperidin-3-yl)amino)pyridin-2-yl)piperidin-4-yl)azetidine-3-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (36)

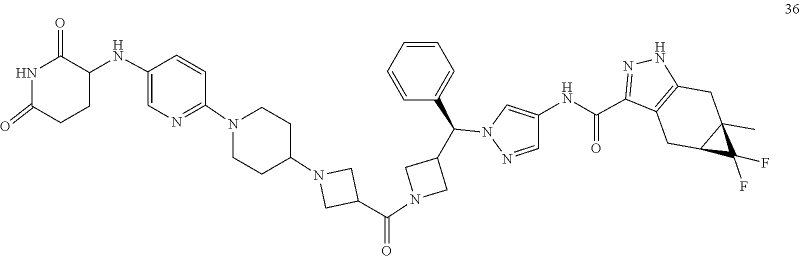 [SMILES: C[C@@]12Cc3[nH]nc(C(=O)Nc4cnn([C@H](c5ccccc5)C5CN(C(=O)C6CN(C7CCN(c8ccc(NC9CCC(=O)NC9=O)cn8)CC7)C6)C5)c4)c3C[C@@H]1C2(F)F]

Using general procedure 7, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (26 mg, 0.060 mmol) was treated with 1-(1-(5-((2,6-dioxopiperidin-3-yl)amino)pyridin-2-yl)piperidin-4-yl)azetidine-3-carboxylic acid (23 mg, 0.060 mmol) to afford the desired product (4.9 mg, 0.01 mmol, 10%). ¹H NMR (500 MHz, CD₃CN) δ 10.99 (s, 1H), 8.86 (d, J=5.7 Hz, 1H), 8.66 (s, 1H), 7.99 (s, 1H), 7.71 (d, J=3.0 Hz, 1H), 7.61 (d, J=3.6 Hz, 1H), 7.37 (t, J=2.8 Hz, 3H), 7.04 (dd, J=9.0, 3.0 Hz, 1H), 6.66 (d, J=9.0 Hz, 1H), 5.51 (d, J=10.4 Hz, 1H), 5.45 (s, 1H), 4.40 (d, J=6.1 Hz, 1H), 4.16 (t, J=8.4 Hz, OH), 4.07 (dd, J=11.9, 5.2 Hz, 1H), 4.01-3.84 (m, 3H), 3.75-3.59 (m, 2H), 3.19-3.03 (m, 3H), 2.82-2.61 (m, 4H), 2.32-2.24 (m, 1H), 1.83-1.73 (m, 2H), 1.66 (dd, J=14.7, 6.9 Hz, 1H), 1.37 (s, 3H), 1.27 (s, 1H). LCMS: C₄₂H₄₇F₂N₁₁O₄ requires: 808, found: m/z=809 [M+H]⁺.

### Example 182. (4aS,5aR)-N-(1-((1S)-(1-(1-(1-(6-((2,6-dioxopiperidin-3-yl)carbamoyl)pyridin-; 3-yl)piperidin-4-yl)azetidine-3-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (37)

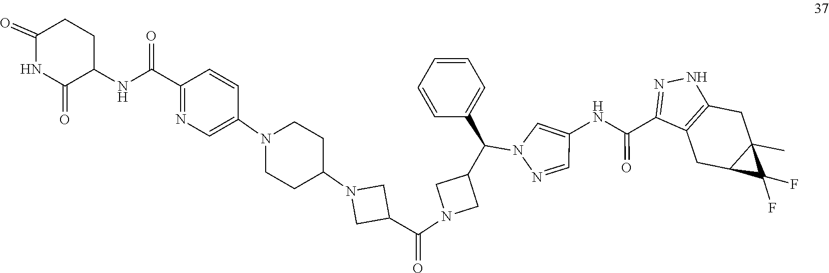 [SMILES: C[C@@]12Cc3[nH]nc(C(=O)Nc4cnn([C@H](c5ccccc5)C5CN(C(=O)C6CN(C7CCN(c8ccc(C(=O)NC9CCC(=O)NC9=O)nc8)CC7)C6)C5)c4)c3C[C@@H]1C2(F)F]

Using general procedure 9_{τ}(4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (9 mg, 0.020 mmol) was treated with 1-(1-(6-((2,6-dioxopiperidin-3-yl)carbamoyl)pyridin-3-yl)piperidin-4-yl)azetidine-3-carboxylic acid (8.6 mg, 0.020 mmol) to afford the desired product (4.7 mg, 0.01 mmol, 26%). ¹H NMR (500 MHz, CD₃CN) δ 10.99 (s, 1H), 8.86 (d, J=4.4 Hz, 1H), 8.75 (s, 1H), 8.26 (d, J=7.7 Hz, 1H), 8.22 (d, J=3.0 Hz, 1H), 7.99 (d, J=1.9 Hz, 1H), 7.88 (d, J=8.8 Hz, 1H), 7.61 (d, J=4.0 Hz, 1H), 7.39-7.30 (m, 5H), 7.28 (dd, J=8.8, 2.9 Hz, 1H), 5.51 (d, J=10.3 Hz, 1H), 5.45 (s, 2H), 4.72 (ddd, J=13.0, 7.8, 5.3 Hz, 1H), 4.15 (t, J=8.4 Hz, 1H), 4.10-4.02 (m, 1H), 4.00-3.94 (m, 1H), 3.90-3.82 (m, 1H), 3.71 (tdd, J=16.7, 11.6, 5.6 Hz, 4H), 3.60 (dd, J=9.6, 5.7 Hz, 1H), 3.39 (d, J=8.9 Hz, 2H), 3.20-2.96 (m, 8H), 2.81-2.61 (m, 3H), 2.27 (dddd, J=17.7, 12.6, 6.8, 3.7 Hz, 2H), 1.74 (dt, J=11.7, 4.1 Hz, 2H), 1.70-1.62 (m, 1H), 1.37 (s, 3H), 1.28 (d, J=5.8 Hz, 7H), 0.88 (q, J=5.7, 5.1 Hz, 1H). LCMS: C₄₃H₄₇F₂N₁₁O₅ requires: 836, found: m/z=837 [M+H]⁺.

### Example 183. (4aS,5aR)-N-(1-((1S)-(1-(1-(2-(2,6-dioxopiperidin-3-yl)-1,3-dioxoisoindolin-5-yl)piperidine-4-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (38)

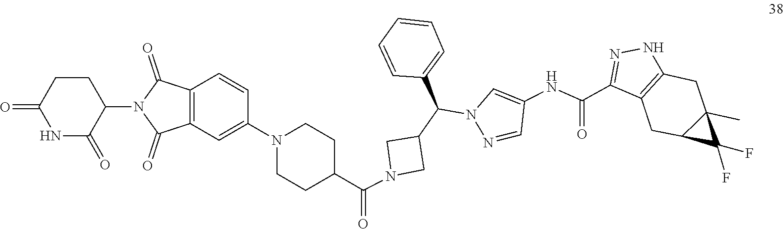 [SMILES: C[C@@]12Cc3[nH]nc(C(=O)Nc4cnn([C@H](c5ccccc5)C5CN(C(=O)C6CCN(c7ccc8c(c7)C(=O)N(C7CCC(=O)NC7=O)C8=O)CC6)C5)c4)c3C[C@@H]1C2(F)F]

Using general procedure 14, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (40 mg, 0.090 mmol) was treated with tert-butyl 1-(2-(2,6-dioxopiperidin-3-yl)-1,3-dioxoisoindolin-5-yl)piperidine-4-carboxylate (40 mg, 0.090 mmol) to afford the desired product (5.7 mg, 0.01 mmol, 7.4%). ¹H NMR (500 MHz, CD₃CN) δ 8.90-8.84 (m, 2H), 8.02 (d, J=3.9 Hz, 1H), 7.70 (d, J=8.4 Hz, 1H), 7.63 (s, 1H), 7.44-7.33 (m, 6H), 7.30 (s, 1H), 5.55 (d, J=10.7 Hz, 1H), 4.95 (dd, J=12.3, 5.4 Hz, 1H), 4.26 (dt, J=51.7, 8.5 Hz, 1H), 3.94 (td, J=25.2, 24.5, 8.1 Hz, 4H), 3.20-3.02 (m, 5H), 2.71 (dddd, J=31.0, 26.6, 12.7, 6.5 Hz, 4H), 2.13-2.06 (m, 1H), 1.77 (d, J=23.0 Hz, 4H), 1.66 (dd, J=15.1, 7.2 Hz, 1H), 1.37 (s, 3H). LCMS: C₄₂H₄₁F₂N₉O₆ requires: 806, found: m/z=807 [M+H]⁺.

### Example 184. (4aS,5aR)-N-(1-(1-(1-(1-(1-(2-(2,6-dioxopiperidin-3-yl)-1,3-dioxoisoindolin-5-yl)piperidin-4-yl)azetidine-3-carbonyl)azetidin-3-yl)ethyl)-1H-pyrazol-4-yl)-5,5-difluoro-5a-methyl-1,4,4a,5,5a,6-hexahydrocyclopropa[f]indazole-3-carboxamide (39)

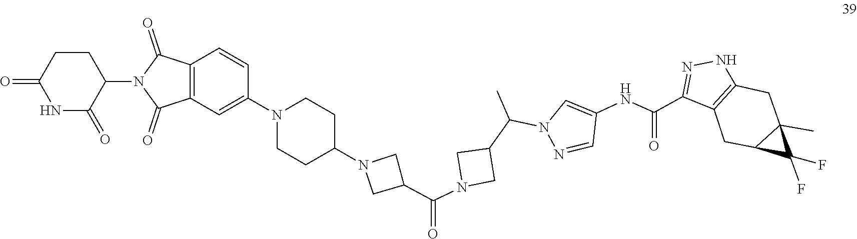 [SMILES: CC(C1CN(C(=O)C2CN(C3CCN(c4ccc5c(c4)C(=O)N(C4CCC(=O)NC4=O)C5=O)CC3)C2)C1)n1cc(NC(=O)c2n[nH]c3c2C[C@@H]2C(F)(F)[C@]2(C)C3)cn1]

Using general procedure 1, (4aS,5aR)-N-(1-(1-(azetidin-3-yl)ethyl)-1H-pyrazol-4-yl)-5,5-difluoro-5a-methyl-1,4,4a,5,5a,6-hexahydrocyclopropa[f]indazole-3-carboxamide (4 mg, 0.010 mmol) was treated with 1-{1-[2-(2,6-dioxopiperidin-3-yl)-1,3-dioxoisoindol-5-yl]piperidin-4-yl}azetidine-3-carboxylic acid (5 mg, 0.010 mmol) to afford the desired product (3.6 mg, 0.045 mmol, 37%). LCMS: C₄₀H₄₄F₂N₁₀O₆ requires: 799, found: m/z=800 [M+H]⁺.

### Example 185. (4aS,5aR)-N-(1-((1S)-(1-(1-(6-((2,6-dioxopiperidin-3-yl)carbamoyl)pyridin-3-yl)piperidine-4-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (40)

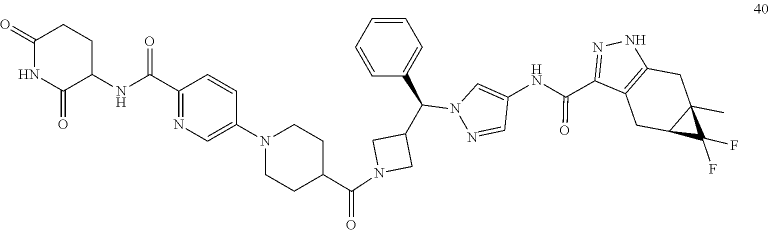 [SMILES: C[C@@]12Cc3[nH]nc(C(=O)Nc4cnn([C@H](c5ccccc5)C5CN(C(=O)C6CCN(c7ccc(C(=O)NC8CCC(=O)NC8=O)nc7)CC6)C5)c4)c3C[C@@H]1C2(F)F]

Using general procedure 1, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (75 mg, 0.17 mmol) was treated with 1-(6-((2,6-dioxopiperidin-3-yl)carbamoyl)pyridin-3-yl)piperidine-4-carboxylic acid (61 mg, 0.17 mmol) to afford the desired product (41 mg, 0.05 mmol, 29%). ¹H NMR (500 MHz, CD₃CN) δ 8.85 (d, J=7.0 Hz, 1H), 8.75 (s, 1H), 8.29-8.21 (m, 2H), 8.01 (d, J=6.3 Hz, 1H), 7.90 (d, J=8.8 Hz, 1H), 7.62 (s, 1H), 7.38 (t, J=5.2 Hz, 4H), 7.32 (d, J=10.2 Hz, 2H), 5.54 (d, J=10.8 Hz, 1H), 4.72 (dt, J=13.0, 6.5 Hz, 1H), 4.30 (t, J=8.3 Hz, 1H), 4.20 (t, J=8.5 Hz, 1H), 3.99 (q, J=9.2, 7.9 Hz, 1H), 3.90 (d, J=13.6 Hz, 3H), 3.71 (dd, J=20.9, 12.4 Hz, 1H), 3.60 (dd, J=9.5, 5.6 Hz, 1H), 3.16 (d, J=17.6 Hz, 1H), 3.12-3.05 (m, 2H), 2.93 (t, J=11.7 Hz, 2H), 2.81-2.61 (m, 3H), 2.51 (s, 1H), 2.12 (dd, J=13.0, 4.7 Hz, 1H), 1.82-1.61 (m, 5H), 1.37 (s, 3H). LCMS: C₄₀H₄₂F₂N₁₀O₅ requires: 781, found: m/z=782 [M+H]⁺.

### Example 186. (4aS,5aR)-N-(1-((1S)-(1-((4-(2-(2,6-dioxopiperidin-3-yl)-1,3-dioxoisoindolin-5-yl)morpholin-2-yl)methyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (41)

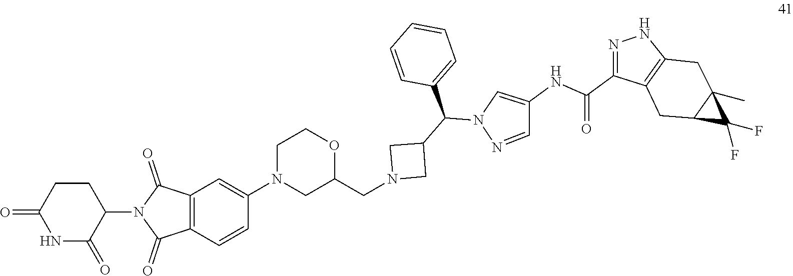 [SMILES: C[C@@]12Cc3[nH]nc(C(=O)Nc4cnn([C@H](c5ccccc5)C5CN(CC6CN(c7ccc8c(c7)C(=O)N(C7CCC(=O)NC7=O)C8=O)CCO6)C5)c4)c3C[C@@H]1C2(F)F]

Using general procedure 3, (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (20 mg, 0.05 mmol) was treated with (2S)-4-(2-(2,6-dioxopiperidin-3-yl)-1,3-dioxoisoindolin-5-yl)morpholine-2-carbaldehyde (17 mg, 0.05 mmol) to afford the desired product (5 mg, 0.01 mmol, 13%). ¹H NMR (500 MHz, CD₃CN) S 8.89 (d, J=10.1 Hz, 2H), 7.99 (d, J=54.5 Hz, 1H), 7.71-7.65 (m, 2H), 7.54-7.45 (m, 1H), 7.36 (dd, J=39.9, 11.8 Hz, 5H), 7.17 (s, 1H), 5.05-4.91 (m, 2H), 4.29-3.61 (m, 6H), 3.40-2.91 (m, 6H), 2.73 (dq, J=32.6, 12.9, 9.6 Hz, 5H), 1.66 (s, 2H), 1.36 (s, 3H). LCMS: C₄₁H₄₁F₂N₉O₆ requires: 794, found: m/z=795 [M+H]⁺.

### Example 187. (4aS,5aR)-N-(1-((1-(1-(1-(2-(2,6-dioxopiperidin-3-yl)-1,3-dioxoisoindolin-5-yl)piperidin-4-yl)azetidine-3-carbonyl)azetidin-3-yl)methyl)-1H-pyrazol-4-yl)-5,5-difluoro-5a-methyl-1,4,4a,5,5a,6-hexahydrocyclopropa[f]indazole-3-carboxamide (42)

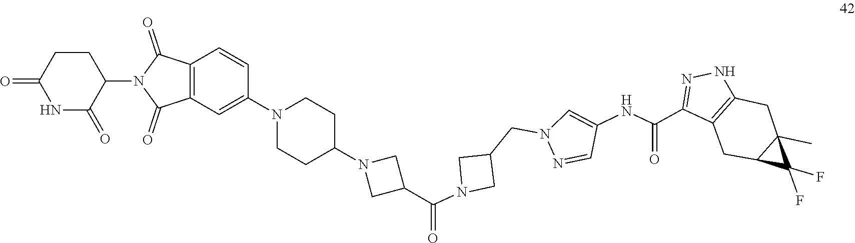 [SMILES: C[C@@]12Cc3[nH]nc(C(=O)Nc4cnn(CC5CN(C(=O)C6CN(C7CCN(c8ccc9c(c8)C(=O)N(C8CCC(=O)NC8=O)C9=O)CC7)C6)C5)c4)c3C[C@@H]1C2(F)F]

Using general procedure 1, (4aS,5aR)-N-[1-(azetidin-3-ylmethyl)pyrazol-4-yl]-5,5-difluoro-5a-methyl-1H,4H,4aH,6H-cyclopropafindazole-3-carboxamide (24 mg, 0.07 mmol) was treated with 1-(1-(2-(2,6-dioxopiperidin-3-yl)-1,3-dioxoisoindolin-5-yl)piperidin-4-yl)azetidine-3-carboxylic acid (30 mg, 0.070 mmol) to afford the desired product (17 mg, 0.02 mmol, 31%). ¹H NMR (500 MHz, (CD₃)₂SO) δ 12.98 (s, 1H), 11.08 (s, 1H), 10.81 (d, J=43.7 Hz, 1H), 10.19 (d, J=4.2 Hz, 1H), 8.07 (d, J=2.6 Hz, 1H), 7.71-7.64 (m, 2H), 7.42-7.25 (m, 6H), 5.66 (dd, J=16.7, 7.5 Hz, 1H), 5.07 (dd, J=12.8, 5.4 Hz, 1H), 4.15 (d, J=19.1 Hz, 6H), 3.99-3.35 (m, 3H), 3.19-2.77 (m, 5H), 2.66-2.53 (m, 1H), 2.01 (d, J=15.7 Hz, 2H), 1.81-1.71 (m, 1H), 1.37 (d, J=26.1 Hz, 5H). LCMS: C₃₉H₄₂F₂N₁₀O₆ requires: 785, found: m/z=786 [M+H]⁺.

### Example 188. (4aS,5aR)-N-(1-((1-(1-(2-(2,6-dioxopiperidin-3-yl)-1,3-dioxoisoindolin-5-yl)azetidine-3-carbonyl)azetidin-3-yl)methyl)-1H-pyrazol-4-yl)-5,5-difluoro-5a-methyl-1,4,4a,5,5a,6-hexahydrocyclopropa[f]indazole-3-carboxamide (43)

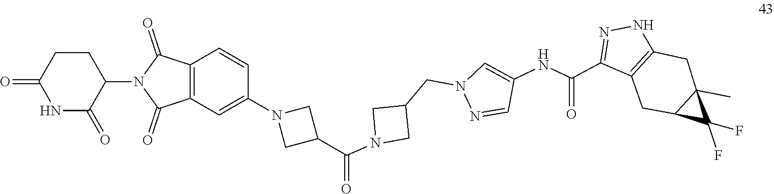 [SMILES: C[C@@]12Cc3[nH]nc(C(=O)Nc4cnn(CC5CN(C(=O)C6CN(c7ccc8c(c7)C(=O)N(C7CCC(=O)NC7=O)C8=O)C6)C5)c4)c3C[C@@H]1C2(F)F]

Using general procedure 1, (4aS,5aR)-N-[1-(azetidin-3-ylmethyl)pyrazol-4-yl]-5,5-difluoro-5a-methyl-1H,4H,4aH,6H-cyclopropafindazole-3-carboxamide (12 mg, 0.03 mmol) was treated with 1-(2-(2,6-dioxopiperidin-3-yl)-1,3-dioxoisoindolin-5-yl)azetidine-3-carboxylic acid (12 mg, 0.03 mmol) to afford the desired product (4.3 mg, 0.01 mmol, 17%). LCMS: C₃₄H₃₃F₂N₉O₆ requires: 702, found: m/z=703 [M+H]⁺.

### Example 189. (4aS,5aR)-N-(1-((1-(((3R)-1-(2-(2,6-dioxopiperidin-3-yl)-1,3-dioxoisoindolin-5-yl)pyrrolidin-3-yl)methyl)azetidin-3-yl)methyl)-1H-pyrazol-4-yl)-5,5-difluoro-5a-methyl-1,4,4a,5,5a,6-hexahydrocyclopropa[f]indazole-3-carboxamide (44)

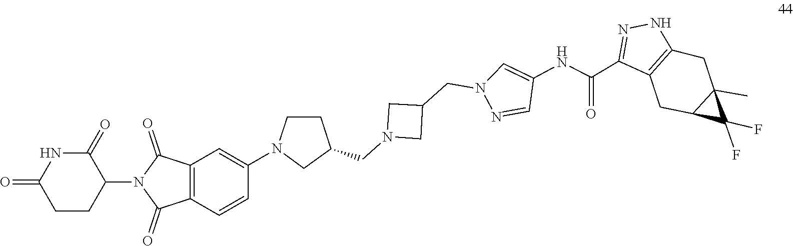 [SMILES: C[C@@]12Cc3[nH]nc(C(=O)Nc4cnn(CC5CN(C[C@H]6CCN(c7ccc8c(c7)C(=O)N(C7CCC(=O)NC7=O)C8=O)C6)C5)c4)c3C[C@@H]1C2(F)F]

Using general procedure 3, (4aS,5aR)-N-[1-(azetidin-3-ylmethyl)pyrazol-4-yl]-5,5-difluoro-5a-methyl-1H,4H,4aH,6H-cyclopropafindazole-3-carboxamide (39 mg, 0.11 mmol) was treated with (3S)-1-(2-(2,6-dioxopiperidin-3-yl)-1,3-dioxoisoindolin-5-yl)pyrrolidine-3-carbaldehyde (38 mg, 0.11 mmol) to afford the desired product (11 mg, 0.01 mmol, 13%). LCMS: C₃₅H₃₇F₂N₉O₅ requires: 702, found: m/z=703 [M+H]⁺.

### Example 190. (S)-6-(4-((4-(2-(6,6-dimethyl-4,5,6,7-tetrahydro-1H-indazol-3-yl)-1H-indole-6-carbonyl)piperazin-1-yl)methyl)piperidin-1-yl)-N-(2,6-dioxopiperidin-3-yl)nicotinamide (45)

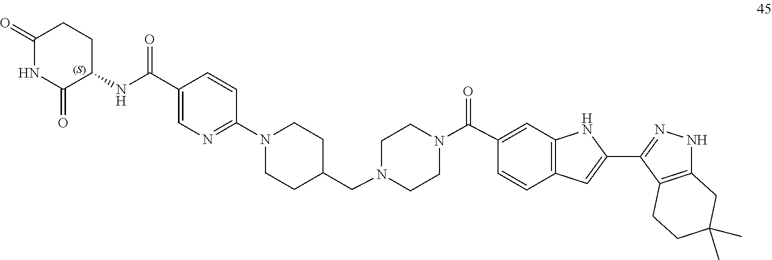

Using general procedure 8, (2-(6,6-dimethyl-4,5,6,7-tetrahydro-1H-indazol-3-yl)-1H-indol-6-yl)(piperazin-1-yl)methanone (25 mg, 0.07 mmol) was treated with (S)—N-(2,6-dioxopiperidin-3-yl)-6-(4-formylpiperidin-1-yl)nicotinamide (24 mg, 0.07 mmol) to afford the desired product (6.1 mg, 0.01 mmol, 12%). ¹H NMR (500 MHz, (CD₃)₂SO) δ 11.54 (s, 1H), 10.86 (d, J=4.0 Hz, 2H), 9.42 (s, 1H), 8.71 (dd, J=8.3, 4.2 Hz, 2H), 8.33 (dd, J=16.4, 2.9 Hz, 2H), 7.87 (t, J=8.9 Hz, 2H), 7.58 (d, J=8.1 Hz, 1H), 7.55 (s, 1H), 7.43 (ddd, J=21.1, 9.1, 2.9 Hz, 2H), 7.10 (d, J=8.0 Hz, 1H), 6.66 (s, 1H), 4.83 (d, J=5.4 Hz, 1H), 4.75 (ddd, J=13.1, 7.9, 5.3 Hz, 2H), 3.99 (t, J=13.9 Hz, 3H), 3.12 (s, 4H), 2.87 (dt, J=42.7, 13.0 Hz, 4H), 2.69 (t, J=6.5 Hz, 2H), 2.55 (s, 2H), 2.43 (s, 2H), 2.18 (qd, J=12.8, 4.1 Hz, 1H), 2.06-1.99 (m, 1H), 1.83 (dd, J=22.9, 12.8 Hz, 4H), 1.59 (t, J=6.3 Hz, 2H), 1.48-1.28 (m, 3H), 1.02 (s, 6H). LCMS: C₃₉H₄₇N₉O₄ requires: 706, found: m/z=707 [M+H]⁺.

### Example 191. (S)-3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-3-methyl-2-oxo-2,3-dihydro-1H-benzo[d]imidazol-1-yl)piperidine-2,6-dione (46)

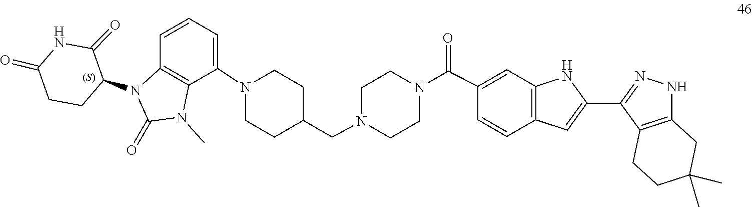

Using general procedure 8, (2-(6,6-dimethyl-4,5,6,7-tetrahydro-1H-indazol-3-yl)-1H-indol-6-yl)(piperazin-1-yl)methanone (30 mg, 0.08 mmol) was treated with (S)-1-(1-(2,6-dioxopiperidin-3-yl)-3-methyl-2-oxo-2,3-dihydro-1H-benzo[d]imidazol-4-yl)piperidine-4-carbaldehyde (30 mg, 0.08 mmol) to afford the desired product (8.6 mg, 0.01 mmol, 13%). ¹H NMR (500 MHz, (CD₃)₂SO) δ 11.53 (s, 1H), 11.09 (s, 1H), 9.44 (s, 1H), 7.60-7.52 (m, 2H), 7.09 (d, J=8.1 Hz, 1H), 6.99 (t, J=7.9 Hz, 1H), 6.89 (t, J=8.0 Hz, 2H), 6.65 (s, 1H), 5.35 (dd, J=12.7, 5.4 Hz, 1H), 3.63 (s, 3H), 3.15 (s, 6H), 2.94-2.84 (m, 1H), 2.77-2.58 (m, 6H), 2.42 (s, 2H), 1.98 (dd, J=12.8, 6.7 Hz, 2H), 1.87 (d, J=12.4 Hz, 2H), 1.58 (t, J=6.5 Hz, 2H), 1.48 (t, J=12.4 Hz, 2H), 1.01 (s, 6H). LCMS: C₄₁H₄₉N₉O₄ requires: 732, found: m/z=733 [M+H]⁺.

### Example 192. 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-3-yl)piperidine-2,6-dione (47)

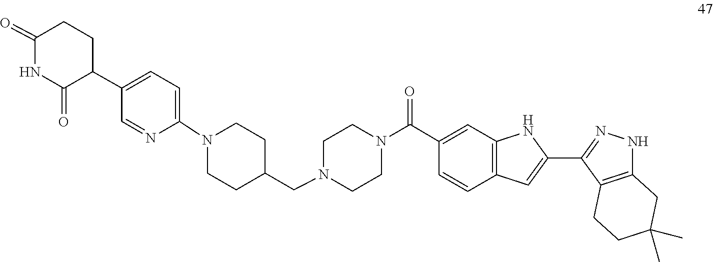 [SMILES: CC1(C)CCc2c(-c3cc4ccc(C(=O)N5CCN(CC6CCN(c7ccc(C8CCC(=O)NC8=O)cn7)CC6)CC5)cc4[nH]3)n[nH]c2C1]

Using general procedure 8, (2-(6,6-dimethyl-4,5,6,7-tetrahydro-1H-indazol-3-yl)-1H-indol-6-yl)(piperazin-1-yl)methanone (103 mg, 0.27 mmol) was treated with 1-(5-(2,6-dioxopiperidin-3-yl)pyridin-2-yl)piperidine-4-carbaldehyde (99 mg, 0.33 mmol) to afford the desired product (141 mg, 0.21 mmol, 76%). ¹H NMR (500 MHz, CDCl₃) δ 10.23 (s, 1H), 10.07 (s, 1H), 8.06 (d, J=2.5 Hz, 1H), 7.59 (d, J=8.1 Hz, 1H), 7.51 (s, 1H), 7.31 (dd, J=8.8, 2.5 Hz, 1H), 7.13 (dd, J=8.1, 1.4 Hz, 1H), 6.73-6.70 (m, 1H), 6.67 (d, J=8.9 Hz, 1H), 4.29 (d, J=12.7 Hz, 2H), 3.68 (dd, J=10.4, 5.2 Hz, 1H), 2.89-2.79 (m, 2H), 2.79-2.74 (m, 2H), 2.73-2.65 (m, 1H), 2.44 (s, 2H), 2.25 (qt, J=14.7, 7.7 Hz, 3H), 1.85 (d, J=13.0 Hz, 2H), 1.77 (s, 1H), 1.65 (t, J=6.3 Hz, 2H), 1.24 (d, J=11.9 Hz, 4H), 1.05 (s, 6H). LCMS: C₃₈H₄₆N₈O₃ requires: 663, found: m/z=664 [M+H]⁺.

### Example 193. (R)-3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-3-methyl-2-oxo-2,3-dihydro-1H-benzo[d]imidazol-1-yl)piperidine-2,6-dione (48)

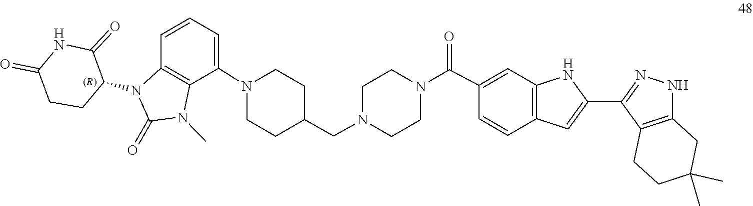

Using general procedure 8, (2-(6,6-dimethyl-4,5,6,7-tetrahydro-1H-indazol-3-yl)-1H-indol-6-yl)(piperazin-1-yl)methanone (30 mg, 0.08 mmol) was treated with (R)-1-(1-(2,6-dioxopiperidin-3-yl)-3-methyl-2-oxo-2,3-dihydro-1H-benzo[d]imidazol-4-yl)piperidine-4-carbaldehyde (30 mg, 0.08 mmol) to afford the desired product (9.2 mg, 0.01 mmol, 16%). ¹H NMR (500 MHz, (CD₃)₂SO) δ 11.54 (s, 1H), 11.10 (s, 1H), 9.46 (s, 1H), 7.59 (d, J=8.2 Hz, 1H), 7.55 (s, 1H), 7.11 (d, J=7.9 Hz, 1H), 7.00 (t, J=7.9 Hz, 1H), 6.91 (t, J=7.9 Hz, 2H), 6.66 (s, 1H), 5.37 (dd, J=12.8, 5.4 Hz, 1H), 3.18 (d, J=10.3 Hz, 6H), 2.90 (q, J=9.6, 5.6 Hz, 1H), 2.79-2.59 (m, 5H), 2.44 (s, 2H), 2.03-1.96 (m, 2H), 1.88 (d, J=12.3 Hz, 2H), 1.60 (t, J=6.4 Hz, 2H), 1.48 (d, J=11.9 Hz, 2H), 1.03 (s, 6H). LCMS: C₄₁H₄₉N₉O₄ requires: 732, found: m/z=733 [M+H]⁺.

### Example 194. (4aS,5aR)-N-(1-((1-(1-(5-(2,6-dioxopiperidin-3-yl)pyridin-2-yl)azetidine-3-carbonyl)azetidin-3-yl)methyl)-1H-pyrazol-4-yl)-5,5-difluoro-5a-methyl-1,4,4a,5,5a,6-hexahydrocyclopropa[f]indazole-3-carboxamide (49)

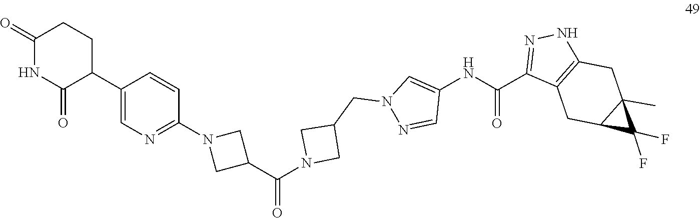 [SMILES: C[C@@]12Cc3[nH]nc(C(=O)Nc4cnn(CC5CN(C(=O)C6CN(c7ccc(C8CCC(=O)NC8=O)cn7)C6)C5)c4)c3C[C@@H]1C2(F)F]

Using general procedure 1, (4aS,5aR)-N-(1-(azetidin-3-ylmethyl)-1H-pyrazol-4-yl)-5,5-difluoro-5a-methyl-1,4,4a,5,5a,6-hexahydrocyclopropafindazole-3-carboxamide (20 mg, 0.06 mmol) was treated with 1-(5-(2,6-dioxopiperidin-3-yl)pyridin-2-yl)azetidine-3-carboxylic acid (16 mg, 0.06 mmol) to afford the desired product (19 mg, 0.03 mmol, 55%). ¹H NMR (500 MHz, CD₃CN) δ 8.91 (s, 1H), 8.83 (s, 1H), 8.01 (d, J=2.1 Hz, 1H), 7.74 (ddd, J=8.9, 5.6, 2.5 Hz, 1H), 7.67 (d, J=2.3 Hz, 1H), 7.61 (s, 1H), 6.72 (dt, J=9.5, 2.4 Hz, 1H), 4.39-4.29 (m, 5H), 4.26 (dd, J=9.1, 5.6 Hz, 1H), 4.16 (t, J=8.5 Hz, 1H), 4.01 (t, J=9.2 Hz, 1H), 3.93 (dd, J=8.7, 5.5 Hz, 1H), 3.82-3.73 (m, 2H), 3.58 (ddd, J=14.6, 8.6, 6.1 Hz, 1H), 3.21-3.05 (m, 4H), 2.79 (dd, J=17.3, 3.5 Hz, 1H), 2.70-2.65 (m, 1H), 2.15 (dddd, J=31.0, 13.0, 9.5, 3.9 Hz, 2H), 1.72-1.63 (m, 1H), 1.38 (d, J=2.6 Hz, 3H). LCMS: C₃₁H₃₃F₂N₉O₄ requires: 634, found: m/z=635 [M+H]⁺.

### Example 195. (4aS,5aR)-N-(1-((1-(1-(5-(2,6-dioxopiperidin-3-yl)pyridin-2-yl)piperidine-4-carbonyl)azetidin-3-yl)methyl)-1H-pyrazol-4-yl)-5,5-difluoro-5a-methyl-1,4,4a,5,5a,6-hexahydrocyclopropa[f]indazole-3-carboxamide (50)

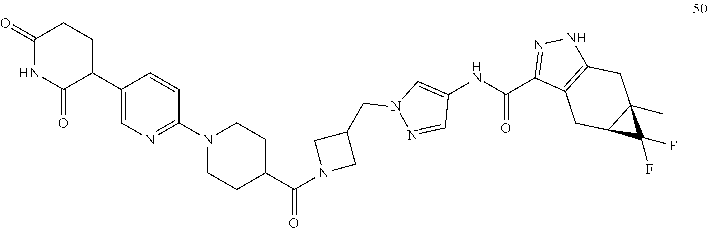 [SMILES: C[C@@]12Cc3[nH]nc(C(=O)Nc4cnn(CC5CN(C(=O)C6CCN(c7ccc(C8CCC(=O)NC8=O)cn7)CC6)C5)c4)c3C[C@@H]1C2(F)F]

Using general procedure 1, (4aS,5aR)-N-(1-(azetidin-3-ylmethyl)-1H-pyrazol-4-yl)-5,5-difluoro-5a-methyl-1,4,4a,5,5a,6-hexahydrocyclopropafindazole-3-carboxamide (27 mg, 0.07 mmol) was treated with 1-(5-(2,6-dioxopiperidin-3-yl)pyridin-2-yl)piperidine-4-carboxylic acid (24 mg, 0.07 mmol) to afford the desired product (19 mg, 0.03 mmol, 38%). ¹H NMR (500 MHz, CD₃CN) δ 8.89 (d, J=4.9 Hz, 1H), 8.83 (d, J=11.0 Hz, 1H), 8.02 (s, 1H), 7.82-7.75 (m, 2H), 7.59 (s, 1H), 7.17 (dd, J=9.6, 2.7 Hz, 1H), 4.31 (d, J=7.0 Hz, 2H), 4.25 (t, J=8.5 Hz, 1H), 4.07-4.00 (m, 3H), 3.95 (t, J=9.2 Hz, 1H), 3.79 (dd, J=12.8, 5.1 Hz, 1H), 3.70 (dd, J=10.0, 5.4 Hz, 1H), 3.34-3.24 (m, 2H), 3.18 (d, J=17.6 Hz, 1H), 3.14-3.04 (m, 3H), 2.79 (dd, J=17.3, 3.4 Hz, 1H), 2.68 (d, J=3.0 Hz, 1H), 2.62 (tt, J=10.1, 4.1 Hz, 1H), 2.27-2.07 (m, 2H), 1.83 (d, J=14.1 Hz, 2H), 1.76-1.63 (m, 3H), 1.37 (d, J=2.5 Hz, 3H). LCMS: C₃₃H₃₇F₂N₉O₄ requires: 662, found: m/z=663 [M+H]⁺.

### Example 196. (4aS,5aR)-N-(1-((1S)-(1-(1-(5-(2,6-dioxopiperidin-3-yl)pyridin-2-yl)azetidine-3-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (51)

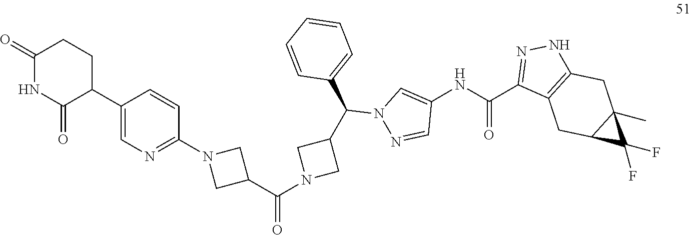 [SMILES: C[C@@]12Cc3[nH]nc(C(=O)Nc4cnn([C@H](c5ccccc5)C5CN(C(=O)C6CN(c7ccc(C8CCC(=O)NC8=O)cn7)C6)C5)c4)c3C[C@@H]1C2(F)F]

Using general procedure 1, starting from (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (23 mg, 0.05 mmol) was treated with 1-(5-(2,6-dioxopiperidin-3-yl)pyridin-2-yl)azetidine-3-carboxylic acid (15 mg, 0.05 mmol) to afford the desired product (20 mg, 0.03 mmol, 54%). ¹H NMR (500 MHz, CD₃CN) δ 8.90 (s, 1H), 8.82 (s, 1H), 8.02 (d, J=4.9 Hz, 1H), 7.78-7.70 (m, 2H), 7.63 (s, 1H), 7.41-7.30 (m, 5H), 6.71 (dd, J=9.2, 5.4 Hz, 1H), 5.57-5.51 (m, 1H), 5.45 (s, 1H), 4.42-4.18 (m, 5H), 4.14-4.01 (m, 1H), 3.99-3.89 (m, 1H), 3.77 (dp, J=11.8, 6.7, 5.9 Hz, 3H), 3.69-3.51 (m, 2H), 3.16 (d, J=17.6 Hz, 1H), 3.12-3.02 (m, 2H), 2.81-2.74 (m, 1H), 2.70-2.62 (m, 2H), 2.17 (dtd, J=13.5, 11.7, 10.1, 5.6 Hz, 1H), 2.14-2.05 (m, 1H), 1.66 (dd, J=15.2, 6.9 Hz, 1H), 1.37 (s, 3H). LCMS: C₃₇H₃₇F₂N₉O₄ requires: 709, found: m/z=710 [M+H]⁺.

### Example 197. 3-(6-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)pyridin-3-yl)piperidine-2,6-dione (52)

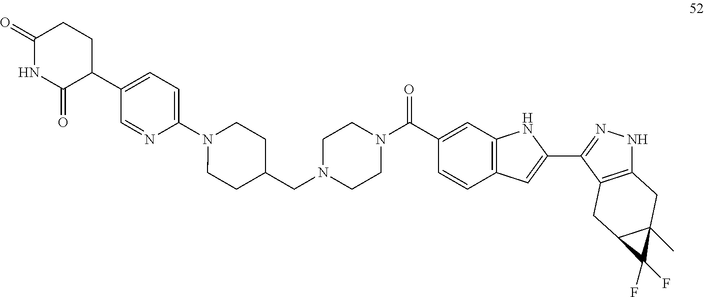 [SMILES: C[C@@]12Cc3[nH]nc(-c4cc5ccc(C(=O)N6CCN(CC7CCN(c8ccc(C9CCC(=O)NC9=O)cn8)CC7)CC6)cc5[nH]4)c3C[C@@H]1C2(F)F]

Using general procedure 8, (2-((4aS,5aR)-5,5-difluoro-5a-methyl-1,4,4a,5,5a,6-hexahydrocyclopropafindazol-3-yl)-1H-indol-6-yl)(piperazin-1-yl)methanone (20 mg, 0.05 mmol) was treated with 1-(5-(2,6-dioxopiperidin-3-yl)pyridin-2-yl)piperidine-4-carbaldehyde (15 mg, 0.05 mmol) to afford the desired product (16 mg, 0.02 mmol, 48%). ¹H NMR (500 MHz, CD₃CN) δ 10.01 (d, J=30.6 Hz, 1H), 8.87 (s, 1H), 7.84-7.77 (m, 2H), 7.64 (d, J=8.1 Hz, 1H), 7.55 (s, 1H), 7.18 (d, J=9.4 Hz, 1H), 7.14 (dd, J=8.2, 1.4 Hz, 1H), 6.72 (d, J=3.5 Hz, 1H), 4.10 (d, J=13.5 Hz, 2H), 3.80 (dd, J=12.8, 5.1 Hz, 1H), 3.53 (s, 3H), 3.25-3.06 (m, 5H), 3.00 (d, J=6.8 Hz, 2H), 2.82 (dd, J=16.8, 3.3 Hz, 1H), 2.70-2.64 (m, 2H), 2.30-2.05 (m, 2H), 2.03-1.96 (m, 2H), 1.81-1.74 (m, 1H), 1.41 (s, 5H). LCMS: C₃₈H₄₂F₂N₈O₃ requires: 696, found: m/z=697 [M+H]⁺.

### Example 198. (4aS,5aR)-N-(1-((1S)-(1-(8-(2-(2,6-dioxopiperidin-3-yl)-1,3-dioxoisoindolin-5-yl)-2,8-diazaspiro[4.5]decane-2-carbonyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (53)

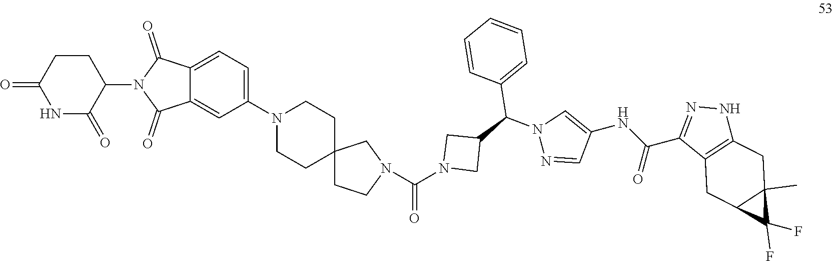 [SMILES: C[C@@]12Cc3[nH]nc(C(=O)Nc4cnn([C@H](c5ccccc5)C5CN(C(=O)N6CCC7(CCN(c8ccc9c(c8)C(=O)N(C8CCC(=O)NC8=O)C9=O)CC7)C6)C5)c4)c3C[C@@H]1C2(F)F]

Using general procedure 12, 2-(2,6-dioxopiperidin-3-yl)-5-(2,8-diazaspiro[4.5]decan-8-yl)isoindoline-1,3-Dione was treated with (4aS,5aR)-N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₃) to afford the desired product. ¹H NMR (500 MHz, DMSO-d₆) δ 12.96 (s, 1H), 11.07 (s, 1H), 10.16 (s, 1H), 8.08 (s, 1H), 7.66 (t, J=4.3 Hz, 2H), 7.46-7.11 (m, 7H), 5.65 (d, J=10.0 Hz, 1H), 5.07 (dd, J=12.8, 5.4 Hz, 1H), 3.89 (d, J=48.1 Hz, 1H), 3.70-3.58 (m, 2H), 3.52 (s, 3H), 3.03 (d, J=20.3 Hz, 4H), 2.85 (dd, J=30.0, 15.4 Hz, 1H), 2.74-2.62 (m, 3H), 2.06-1.96 (m, 1H), 1.83-1.64 (m, 3H), 1.54 (s, 4H), 1.35 (s, 3H), 1.24 (d, J=9.5 Hz, 4H), 0.91 (dd, J=48.2, 6.7 Hz, 1H). LCMS: C₄₅H₄₆F₂N₁₀O₆ requires: 860, found: m/z=861 [M+H]⁺.

### Example 199. 3-(6-(4-((1-(2-(6,6-dimethyl-4,5,6,7-tetrahydro-1H-indazol-3-yl)-1H-indole-6-carbonyl)piperidin-4-yl)methyl)piperidin-1-yl)pyridin-3-yl)piperidine-2,6-dione (54)

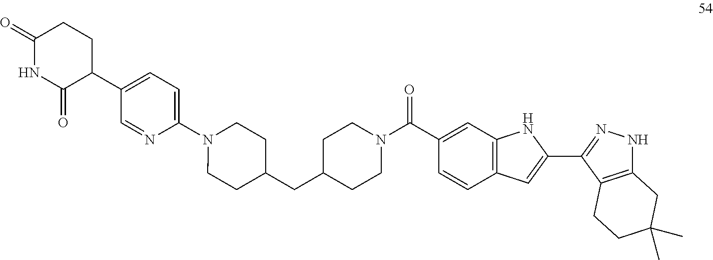 [SMILES: CC1(C)CCc2c(-c3cc4ccc(C(=O)N5CCC(CC6CCN(c7ccc(C8CCC(=O)NC8=O)cn7)CC6)CC5)cc4[nH]3)n[nH]c2C1]

Using general procedure 9, 2-(6,6-dimethyl-4,5,6,7-tetrahydro-1H-indazol-3-yl)-1H-indole-6-carboxylic acid (17 mg, 0.05 mmol) was treated with 3-(6-(4-(piperidin-4-ylmethyl)piperidin-1-yl)pyridin-3-yl)piperidine-2,6-dione (16 mg, 0.04 mmol) to afford the desired product (9.3 mg, 0.01 mmol, 30%). ¹H NMR (500 MHz, (CD₃)₂SO) δ 11.40 (s, 1H), 10.91 (s, 1H), 7.86 (s, 1H), 7.77 (s, 1H), 7.52 (d, J=8.2 Hz, 1H), 7.43 (s, 1H), 7.26 (s, 1H), 6.98 (d, J=8.2 Hz, 1H), 6.61 (s, 1H), 4.18 (d, J=13.0 Hz, 2H), 3.87 (s, 1H), 3.04 (t, J=64.3 Hz, 4H), 2.68 (d, J=5.4 Hz, 3H), 2.56 (d, J=17.7 Hz, 1H), 2.42 (s, 2H), 2.38-2.19 (m, 1H), 1.97 (s, 1H), 1.79 (d, J=12.9 Hz, 2H), 1.69 (s, 6H), 1.57 (d, J=7.0 Hz, 3H), 1.31 (q, J=7.4 Hz, 1H), 1.20 (s, 3H), 1.11 (dd, J=25.4, 12.0 Hz, 2H), 1.01 (s, 6H), 0.94 (t, J=7.3 Hz, 1H). LCMS: C₃₉H₄₇N₇O₃ requires: 662, found: m/z=663 [M+H]⁺.

### Example 200. (4aS,5aR)-N-(1-(1-(1-(5-(2,6-dioxopiperidin-3-yl)pyridin-2-yl)azetidine-3-carbonyl)azetidin-3-yl)-1H-pyrazol-4-yl)-5,5-difluoro-5a-methyl-1,4,4a,5,5a,6-hexahydrocyclopropa[f]indazole-3-carboxamide (55)

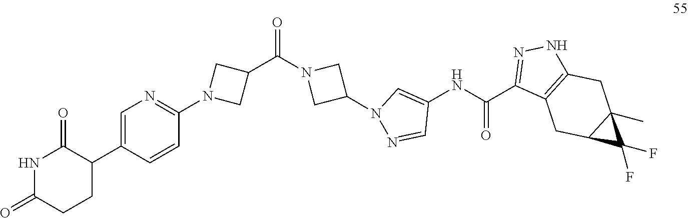 [SMILES: C[C@@]12Cc3[nH]nc(C(=O)Nc4cnn(C5CN(C(=O)C6CN(c7ccc(C8CCC(=O)NC8=O)cn7)C6)C5)c4)c3C[C@@H]1C2(F)F]

Using general procedure 1, (4aS,5aR)-N-(1-(azetidin-3-yl)-1H-pyrazol-4-yl)-5,5-difluoro-5a-methyl-1,4,4a,5,5a,6-hexahydrocyclopropa[f]indazole-3-carboxamide (18 mg, 0.05 mmol) was treated with 1-(5-(2,6-dioxopiperidin-3-yl)pyridin-2-yl)azetidine-3-carboxylic acid (15 mg, 0.05 mmol) to afford the desired product (21 mg, 0.03 mmol, 66%). ¹H NMR (500 MHz, (CD₃)₂SO) δ 12.99 (s, 1H), 10.92 (s, 1H), 10.23 (s, 1H), 8.19 (s, 1H), 7.90 (d, J=2.1 Hz, 1H), 7.84 (s, 1H), 7.77 (s, 1H), 6.84 (s, 1H), 5.27 (ddd, J=13.4, 8.1, 5.3 Hz, 1H), 4.56 (t, J=8.5 Hz, 1H), 4.42-4.32 (m, 4H), 4.24 (s, 2H), 4.15 (dd, J=10.1, 5.3 Hz, 1H), 3.92-3.85 (m, 1H), 3.69 (ddd, J=14.8, 8.7, 6.0 Hz, 1H), 3.12-3.00 (m, 3H), 2.82 (dd, J=17.6, 3.1 Hz, 1H), 2.74-2.62 (m, 1H), 2.61-2.53 (m, 1H), 2.32-2.21 (m, 1H), 2.01-1.94 (m, 1H), 1.78 (d, J=12.7 Hz, 1H), 1.35 (s, 3H). LCMS: C₃₀H₃₁F₂N₉O₄ requires: 619, found: m/z=620 [M+H]⁺.

### Example 201. (4aS,5aR)-N-(1-(1-(1-(1-(2,6-dioxopiperidin-3-yl)-3-methyl-2-oxo-2,3-dihydro-1H-benzo[d]imidazol-4-yl)piperidine-4-carbonyl)azetidin-3-yl)-1H-pyrazol-4-yl)-5,5-difluoro-5a-methyl-1,4,4a,5,5a,6-hexahydrocyclopropa[f]indazole-3-carboxamide (56)

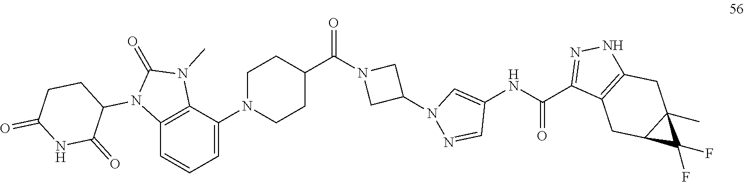 [SMILES: Cn1c(=O)n(C2CCC(=O)NC2=O)c2cccc(N3CCC(C(=O)N4CC(n5cc(NC(=O)c6n[nH]c7c6C[C@@H]6C(F)(F)[C@]6(C)C7)cn5)C4)CC3)c21]

Using general procedure 1, (4aS,5aR)-N-(1-(azetidin-3-yl)-1H-pyrazol-4-yl)-5,5-difluoro-5a-methyl-1,4,4a,5,5a,6-hexahydrocyclopropa[f]indazole-3-carboxamide (18 mg, 0.05 mmol) was treated with 1-(1-(2,6-dioxopiperidin-3-yl)-3-methyl-2-oxo-2,3-dihydro-1H-benzo[d]imidazol-4-yl)piperidine-4-carboxylic acid (16 mg, 0.05 mmol) to afford the desired product (17 mg, 0.03 mmol, 52%). ¹H NMR (500 MHz, (CD₃)₂SO) δ 10.93 (s, 1H), 10.23 (s, 1H), 8.18 (s, 1H), 7.88 (d, J=1.9 Hz, 1H), 7.86 (s, 1H), 7.77 (s, 1H), 7.36 (s, 1H), 5.25 (td, J=8.0, 4.0 Hz, 1H), 4.67 (t, J=8.5 Hz, 1H), 4.49 (dd, J=9.0, 5.2 Hz, 1H), 4.29 (t, J=9.1 Hz, 1H), 4.19 (d, J=13.4 Hz, 2H), 4.09 (dd, J=10.1, 5.3 Hz, 1H), 3.92 (dd, J=12.8, 4.8 Hz, 1H), 3.27-3.15 (m, 2H), 3.13-3.00 (m, 3H), 2.82 (dd, J=16.9, 3.3 Hz, 1H), 2.69 (ddt, J=17.7, 12.9, 5.2 Hz, 2H), 2.57 (dt, J=20.7, 5.5 Hz, 1H), 2.29 (qd, J=13.0, 4.4 Hz, 1H), 1.98 (ddq, J=10.4, 5.3, 2.9 Hz, 1H), 1.87-1.75 (m, 3H), 1.62 (d, J=12.8 Hz, 2H), 1.35 (s, 3H). LCMS: C₃₅H₃₈F₂N₁₀O₅ requires: 716, found: m/z=717 [M+H]⁺.

### Example 202. (4aS,5aR)-N-(1-(1-((1-(1-(2,6-dioxopiperidin-3-yl)-3-methyl-2-oxo-2,3-dihydro-1H-benzo[d]imidazol-4-yl)piperidin-4-yl)methyl)azetidin-3-yl)-1H-pyrazol-4-yl)-5,5-difluoro-5a-methyl-1,4,4a,5,5a,6-hexahydrocyclopropa[f]indazole-3-carboxamide (57)

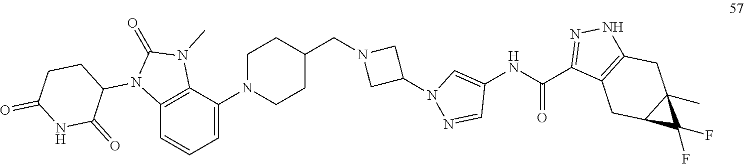 [SMILES: Cn1c(=O)n(C2CCC(=O)NC2=O)c2cccc(N3CCC(CN4CC(n5cc(NC(=O)c6n[nH]c7c6C[C@@H]6C(F)(F)[C@]6(C)C7)cn5)C4)CC3)c21]

Using general procedure 3, (4aS,5aR)-N-(1-(azetidin-3-yl)-1H-pyrazol-4-yl)-5,5-difluoro-5a-methyl-1,4,4a,5,5a,6-hexahydrocyclopropa[f]indazole-3-carboxamide (20 mg, 0.06 mmol) was treated with 1-(1-(2,6-dioxopiperidin-3-yl)-3-methyl-2-oxo-2,3-dihydro-1H-benzo[d]imidazol-4-yl)piperidine-4-carbaldehyde (21 mg, 0.06 mmol) to afford the desired product (14 mg, 0.02 mmol, 34%). ¹H NMR (500 MHz, (CD₃)₂SO) δ 13.01 (s, 1H), 11.08 (s, 1H), 10.29 (d, J=13.4 Hz, 1H), 9.78 (s, 1H), 8.28 (d, J=15.4 Hz, 1H), 7.84 (d, J=5.3 Hz, 1H), 6.98 (t, J=7.9 Hz, 1H), 6.89 (dd, J=13.9, 8.0 Hz, 2H), 5.42-5.31 (m, 2H), 4.69 (s, 1H), 4.65-4.49 (m, 1H), 4.44 (d, J=6.4 Hz, 1H), 3.63 (s, 3H), 3.34 (s, 1H), 3.20-2.99 (m, 5H), 2.94-2.78 (m, 2H), 2.74-2.58 (m, 5H), 2.02-1.94 (m, 1H), 1.79 (t, J=11.8 Hz, 4H), 1.47 (d, J=12.0 Hz, 2H), 1.35 (s, 3H). LCMS: C₃₅H₄₀F₂N₁₀O₄ requires: 702, found: m/z=703 [M+H]⁺.

### Example 203. 3-(6-(1′-(2-(6,6-dimethyl-4,5,6,7-tetrahydro-1H-indazol-3-yl)-1H-indole-6-carbonyl)-[4,4′-bipiperidin]-1-yl)pyridin-3-yl)piperidine-2,6-dione (58)

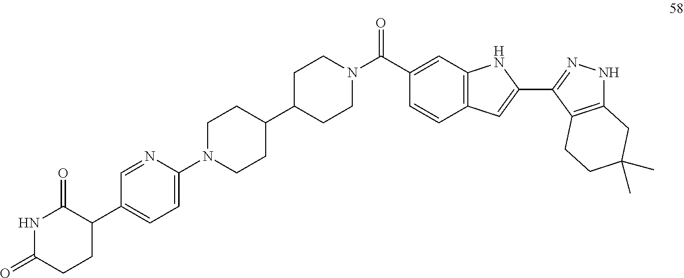 [SMILES: CC1(C)CCc2c(-c3cc4ccc(C(=O)N5CCC(C6CCN(c7ccc(C8CCC(=O)NC8=O)cn7)CC6)CC5)cc4[nH]3)n[nH]c2C1]

Using general procedure 9, 2-(6,6-dimethyl-4,5,6,7-tetrahydro-1H-indazol-3-yl)-1H-indole-6-carboxylic acid (29 mg, 0.09 mmol) was treated with 3-(6-([4,4′-bipiperidin]-1-yl)pyridin-3-yl)piperidine-2,6-dione (28 mg, 0.08 mmol) to afford the desired product (11 mg, 0.02 mmol, 22%). ¹H NMR (500 MHz, (CD₃)₂SO) δ 11.40 (s, 1H), 10.89 (s, 1H), 7.87 (s, 1H), 7.52 (d, J=8.1 Hz, 1H), 7.43 (s, 1H), 6.98 (d, J=8.0 Hz, 1H), 6.61 (s, 1H), 4.24 (d, J=13.0 Hz, 2H), 3.85 (s, 1H), 3.20-3.13 (m, 4H), 2.67 (td, J=14.0, 8.7 Hz, 3H), 2.55 (d, J=14.1 Hz, 1H), 2.42 (s, 2H), 2.25 (d, J=13.3 Hz, 1H), 2.00-1.93 (m, 1H), 1.81 (d, J=11.7 Hz, 2H), 1.72 (s, 1H), 1.57 (p, J=8.0, 7.1 Hz, 6H), 1.41 (s, 1H), 1.36-1.10 (m, 7H), 0.94 (t, J=7.3 Hz, 6H). LCMS: C₃₈H₄₅N₇O₃ requires: 647, found: m/z=648 [M+H]⁺.

### Example 204. 3-(6-(4-((1-(2-(6,6-dimethyl-4,5,6,7-tetrahydro-1H-indazol-3-yl)-1H-indole-6-carbonyl)piperidin-4-yl)methyl)piperazin-1-yl)pyridin-3-yl)piperidine-2,6-dione (59)

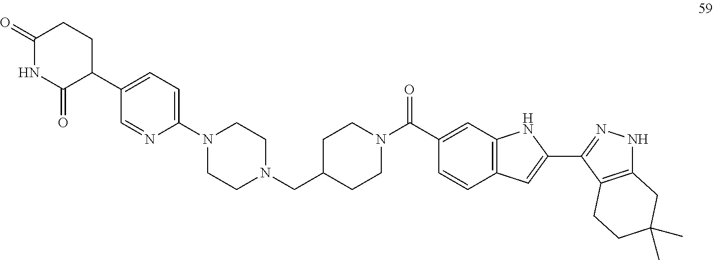 [SMILES: CC1(C)CCc2c(-c3cc4ccc(C(=O)N5CCC(CN6CCN(c7ccc(C8CCC(=O)NC8=O)cn7)CC6)CC5)cc4[nH]3)n[nH]c2C1]

Using general procedure 9, 2-(6,6-dimethyl-4,5,6,7-tetrahydro-1H-indazol-3-yl)-1H-indole-6-carboxylic acid (29 mg, 0.09 mmol) was treated with 3-(6-(4-(piperidin-4-ylmethyl)piperazin-1-yl)pyridin-3-yl)piperidine-2,6-dione (29 mg, 0.08 mmol) to afford the desired product (14 mg, 0.02 mmol, 27%). ¹H NMR (500 MHz, (CD₃)₂SO) δ 11.45 (s, 1H), 10.84 (s, 1H), 9.34 (s, 1H), 8.03 (d, J=2.4 Hz, 1H), 7.56-7.49 (m, 2H), 7.44 (s, 1H), 7.02-6.94 (m, 2H), 6.62 (d, J=2.0 Hz, 1H), 4.35 (d, J=13.7 Hz, 2H), 3.79 (dd, J=12.4, 4.9 Hz, 1H), 3.61 (d, J=11.8 Hz, 2H), 3.21 (t, J=12.7 Hz, 2H), 3.10 (dd, J=18.2, 8.2 Hz, 4H), 2.73-2.62 (m, 3H), 2.54 (s, 1H), 2.50-2.44 (m, 3H), 2.42 (s, 2H), 2.19 (ddd, J=25.6, 13.9, 8.0 Hz, 2H), 2.00-1.93 (m, 1H), 1.77 (d, J=19.2 Hz, 2H), 1.58 (t, J=6.4 Hz, 2H), 1.22 (d, J=13.4 Hz, 3H), 1.01 (s, 6H). LCMS: C₃₈H₄₆F₂N₈O₃ requires: 662, found: m/z=663 [M+H]⁺.

### Example 205. 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-3-yl)-1-methylpiperidine-2,6-dione (60)

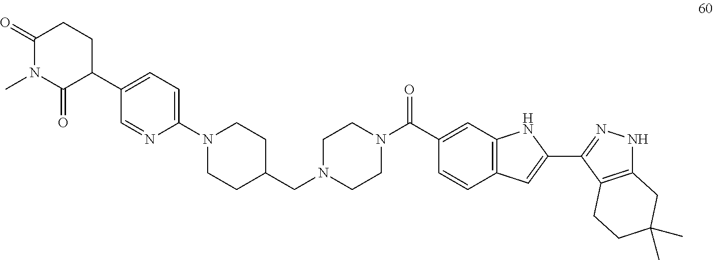 [SMILES: CN1C(=O)CCC(c2ccc(N3CCC(CN4CCN(C(=O)c5ccc6cc(-c7n[nH]c8c7CCC(C)(C)C8)[nH]c6c5)CC4)CC3)nc2)C1=O]

A mixture of 1-(5-(1-methyl-2,6-dioxopiperidin-3-yl)pyridin-2-yl)piperidine-4-carbaldehyde (50 mg, 0.16 mmol), DCM (1.60 mL), IPA (1.60 mL), and (2-(6,6-dimethyl-4,5,6,7-tetrahydro-1H-indazol-3-yl)-1H-indol-6-yl)(piperazin-1-yl)methanone (59.85 mg, 0.16 mmol) was treated with sodium triacetoxyborohydride (67.2 mg, 0.32 mmol). After sixty minutes, the reaction was purified by silica gel column chromatography (0-10% MeOH in DCM, step gradient). The desired compound was re-purified by RP-HPLC (10-95% MeCN in water wth 0.1% TFA) to provide the desired product (15.2 mg, 14.2%). ¹H NMR (500 MHz, CDCl₃) δ 10.23 (s, 1H), 10.07 (s, 1H), 8.06 (d, J=2.5 Hz, 1H), 7.59 (d, J=8.1 Hz, 1H), 7.51 (s, 1H), 7.31 (dd, J=8.8, 2.5 Hz, 1H), 7.13 (dd, J=8.1, 1.4 Hz, 1H), 6.73-6.70 (m, 1H), 6.67 (d, J=8.9 Hz, 1H), 4.29 (d, J=12.7 Hz, 2H), 3.68 (dd, J=10.4, 5.2 Hz, 1H), 2.89-2.79 (m, 2H), 2.79-2.74 (m, 2H), 2.73-2.65 (m, 1H), 2.44 (s, 2H), 2.25 (qt, J=14.7, 7.7 Hz, 3H), 1.85 (d, J=13.0 Hz, 2H), 1.77 (s, 1H), 1.65 (t, J=6.3 Hz, 2H), 1.24 (d, J=11.9 Hz, 4H), 1.05 (s, 6H). LCMS: C₃₉H₄₈N₈O₃ requires 676.4, found: m/z=677.7 [M+H]⁺.

### Example 206. 3-(6-(6-((1-(2-(6,6-dimethyl-4,5,6,7-tetrahydro-1H-indazol-3-yl)-1H-indole-6-carbonyl)piperidin-4-yl)methyl)-2,6-diazaspiro[3.4]octan-2-yl)pyridin-3-yl)piperidine-2,6-dione (61)

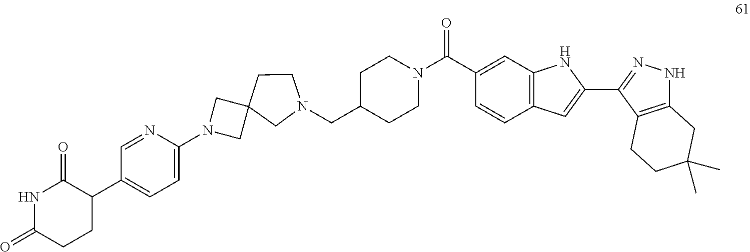 [SMILES: CC1(C)CCc2c(-c3cc4ccc(C(=O)N5CCC(CN6CCC7(C6)CN(c6ccc(C8CCC(=O)NC8=O)cn6)C7)CC5)cc4[nH]3)n[nH]c2C1]

A mixture of 2-(6,6-dimethyl-4,5,6,7-tetrahydro-1H-indazol-3-yl)-1H-indole-6-carboxylic acid (45 mg, 0.15 mmol), DMF (0.99 mL), N,N-diisopropylethylamine (127 μL, 0.09 g, 0.73 mmol), and HATU (61 mg, 0.16 mmol) was treated with a solution of 3-(6-(6-(piperidin-4-ylmethyl)-2,6-diazaspiro[3.4]octan-2-yl)pyridin-3-yl)piperidine-2,6-dione (58 mg, 0.15 mmol) and N,N-diisopropylethylamine (127 μL, 0.09 g, 0.73 mmol) in DMF (0.99 mL). After thirty minutes, the reaction was concentrated under reduced pressure and the residue was purified by RP-HPLC (10-95% MeCN in water) to give the desired product (0.035 g, 35%). ¹H NMR (500 MHz, (CD₃)₂SO) δ 12.57 (s, 1H), 11.43 (s, 1H), 10.80 (s, 1H), 7.91 (s, 1H), 7.53 (d, J=1.9 Hz, 1H), 7.44 (s, 1H), 7.38 (d, J=8.0 Hz, 1H), 7.00 (d, 1H), 6.62 (s, 1H), 6.37 (d, J=8.5 Hz, 1H), 3.87-3.81 (m, 3H), 3.73 (dd, J=12.6, 4.7 Hz, 1H), 3.07-2.79 (m, 2H), 2.78-2.62 (m, 5H), 2.45-2.40 (m, 3H), 2.30 (s, 1H), 2.20-2.12 (m, 1H), 2.09-1.91 (m, 3H), 1.77 (s, 3H), 1.62-1.56 (m, 2H), 1.25 (s, 1H), 1.16-1.05 (m, 2H), 1.02 (s, 6H). LCMS: C₄₀H₄₈N₈O₃ requires: 688.4, found: m/z=689.8 [M+H]⁺.

### Example 207. 3-(6-((1-((1-(2-(6,6-dimethyl-4,5,6,7-tetrahydro-1H-indazol-3-yl)-1H-indole-6-carbonyl)piperidin-4-yl)methyl)piperidin-4-yl)(methyl)amino)pyridin-3-yl)piperidine-2,6-dione (62)

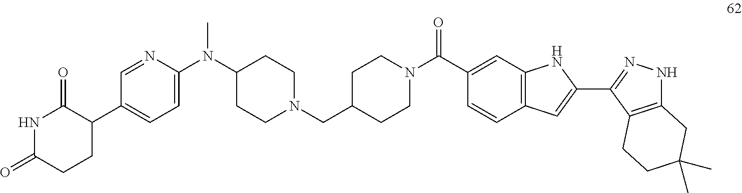 [SMILES: CN(c1ccc(C2CCC(=O)NC2=O)cn1)C1CCN(CC2CCN(C(=O)c3ccc4cc(-c5n[nH]c6c5CCC(C)(C)C6)[nH]c4c3)CC2)CC1]

A mixture of 3-(6-(methyl(1-(piperidin-4-ylmethyl)piperidin-4-yl)amino)pyridin-3-yl)piperidine-2,6-dione (20 mg, 0.05 mmol), DMF (0.5 mL) was added to a mixture of 2-(6,6-dimethyl-4,5,6,7-tetrahydro-1H-indazol-3-yl)-1H-indole-6-carboxylic acid (15 mg, 0.05 mmol), DMF (0.5 mL), N,N-diisopropylethylamine (8.7 μL, 0.05 mmol), and HATU (21 mg, 0.06 mmol) and the reaction was allowed to stir at rt for thirty minutes. The reaction was purified by RP-HPLC (10-80% MeCN in water with 0.1% TFA). The desired freebase was extracted from fractions using DCM and saturated Na₂CO₃. The organic layer was then concentrated under reduced pressure and lyophilized from MeCN:water to give the desired product (0.01 g, 0.01 mmol, 29%). ¹H NMR (500 MHz, (CD₃)₂SO) δ 12.57 (s, 1H), 11.43 (s, 1H), 10.81 (s, 1H), 7.94 (d, J=2.5 Hz, 1H), 7.52 (d, J=8.1 Hz, 1H), 7.43 (s, 1H), 7.37 (dd, J=8.8, 2.5 Hz, 1H), 6.98 (d, J=8.0 Hz, 1H), 6.81 (d, J=8.8 Hz, 1H), 6.61 (s, 1H), 4.33 (d, J=13.1 Hz, 2H), 3.73 (dd, J=12.1, 4.9 Hz, 1H), 2.98-2.82 (m, 2H), 2.79-2.65 (m, 6H), 2.43 (s, 2H), 2.27 (d, J=6.5 Hz, 2H), 2.19 (s, 3H), 2.02-1.95 (m, 1H), 1.76-1.70 (m, 4H), 1.59 (t, J=6.4 Hz, 2H), 1.37 (dd, J=14.1, 10.2 Hz, 2H), 1.25 (s, 1H), 1.06 (s, 2H), 1.02 (s, 6H). LCMS: C₄₀H₅₀N₈O₃ requires: 690.9, found: m/z=691.8 [M+H]⁺.

### Example 208. 3-(6-(4-(((1-(2-(6,6-dimethyl-4,5,6,7-tetrahydro-1H-indazol-3-yl)-1H-indole-6-carbonyl)piperidin-4-yl)methyl)(methyl)amino)piperidin-1-yl)pyridin-3-yl)piperidine-2,6-dione (63)

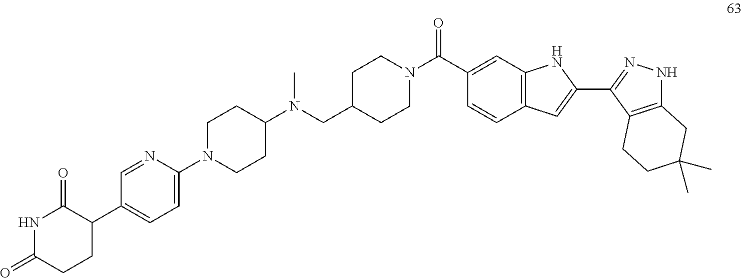 [SMILES: CN(CC1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1]

A mixture of 3-(6-(4-(methyl(piperidin-4-ylmethyl)amino)piperidin-1-yl)pyridin-3-yl)piperidine-2,6-dione (78.0 mg, 0.195 mmol), DMF (0.80 mL), and N,N-diisopropylethylamine (170 μL, 0.130 g, 0.976 mmol) was added to a mixture of 2-(6,6-dimethyl-4,5,6,7-tetrahydro-1H-indazol-3-yl)-1H-indole-6-carboxylic acid (60.4 mg, 0.195 mmol), DMF (0.80 mL), N,N-diisopropylethylamine (170 μL, 0.130 g, 0.976 mmol), and HATU (81.6 mg, 0.215 mmol) and the reaction was allowed to stir at rt for thirty minutes. The reaction was concentrated under reduced pressure and then purified by RP-HPLC (10-80% MeCN in water with 0.1% TFA). The desired freebase was extracted from fractions using DCM and saturated Na₂CO₃. The organic layer was then concentrated under reduced pressure and lyophilized from MeCN:water to give the desired product (0.017 g, mmol, 12.6%). ¹H NMR (500 MHz, (CD₃)₂SO) δ 12.57 (s, 1H), 11.43 (s, 1H), 10.81 (s, 1H), 7.94 (s, 1H), 7.53 (d, J=8.0 Hz, 1H), 7.43 (s, 1H), 7.37 (d, J=2.5 Hz, 1H), 6.98 (d, J=8.1 Hz, 1H), 6.81 (d, J=8.9 Hz, 1H), 6.61 (s, 1H), 4.33 (d, J=12.8 Hz, 2H), 3.73 (dd, J=12.1, 4.9 Hz, 1H), 2.75 (t, J=12.5 Hz, 3H), 2.71-2.65 (m, 3H), 2.43 (s, 2H), 2.27 (d, J=6.5 Hz, 2H), 2.19 (s, 3H), 2.18-2.13 (m, 1H), 2.02-1.95 (m, 1H), 1.73 (d, J=11.8 Hz, 4H), 1.59 (t, J=6.4 Hz, 2H), 1.43-1.33 (m, 2H), 1.11-1.04 (m, 2H), 1.02 (s, 6H). LCMS: C₄₀H₅₀N₈O₃ requires: 690.4, found: m/z=691.7 [M+H]⁺.

### Example 209. 1-(2-(6,6-dimethyl-4,5,6,7-tetrahydro-1H-indazol-3-yl)-1H-indole-6-carbonyl)-N-(1-(5-(2,6-dioxopiperidin-3-yl)pyridin-2-yl)piperidin-4-yl)-N-methylpiperidine-4-carboxamide (64)

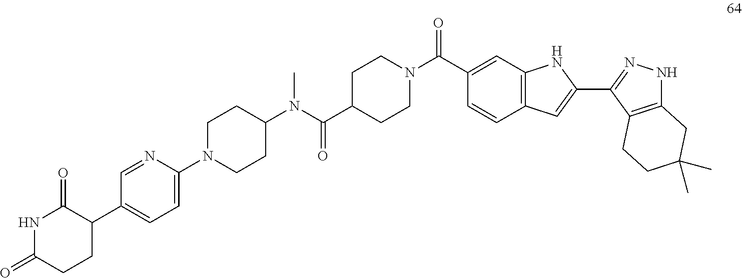 [SMILES: CN(C(=O)C1CCN(C(=O)c2ccc3cc(-c4n[nH]c5c4CCC(C)(C)C5)[nH]c3c2)CC1)C1CCN(c2ccc(C3CCC(=O)NC3=O)cn2)CC1]

A mixture of 2-(6,6-dimethyl-4,5,6,7-tetrahydro-1H-indazol-3-yl)-1H-indole-6-carboxylic acid (74.81 mg, 0.24 mmol), DMF (1.45 mL), N,N-diisopropylethylamine (210.61 μL, 0.16 g, 1.21 mmol), and HATU (91.95 mg, 0.24 mmol) was treated with a mixture of N-(1-(5-(2,6-dioxopiperidin-3-yl)pyridin-2-yl)piperidin-4-yl)-N-methylpiperidine-4-carboxamide (100.00 mg, 0.24 mmol), N,N-diisopropylethylamine (210.61 μL, 0.16 g, 1.21 mmol), and DMF (1.45 mL). After thirty minutes, the reaction was concentrated under reduced pressure and the residue was purified by RP-HPLC (10-95% MeCN in water with 0.1% TFA) to give the desired product (0.016 g, 9.4%). LCMS: C₄₀H₄₈N₈O₄ requires: 705, found: m/z=706 [M+H]⁺.

### Example 210. (2S)—N-(2-((4aS,5aR)-5,5-difluoro-5a-methyl-1,4,4a,5,5a,6-hexahydrocyclopropa[f]indazol-3-yl)-1H-indol-6-yl)-2-(4-((1-(1-(2,6-dioxopiperidin-3-yl)-3-methyl-2-oxo-2,3-dihydro-1H-benzo[d]imidazol-4-yl)piperidin-4-yl)methyl)piperazin-1-yl)-N-methylpropanamide (65)

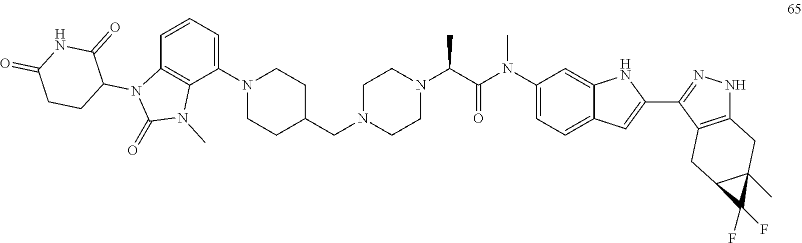 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3cccc4c3n(C)c(=O)n4C3CCC(=O)NC3=O)CC2)CC1]

To a mixture of (2S)—N-[2-[(4aR,5aS)-5,5-difluoro-5a-methyl-1H,4H,4aH,6H-cyclopropa[f]indazol-3-yl]-1H-indol-6-yl]-N-methyl-2-(piperazin-1-yl)propanamide (76 mg, 0.162 mmol, 1 equiv) in DCM (3 mL) were added DIEA (84 mg, 0.648 mmol, 4 equiv) and 1-[1-(2,6-dioxopiperidin-3-yl)-3-methyl-2-oxo-1,3-benzodiazol-4-yl]piperidine-4-carbaldehyde (60 mg, 0.162 mmol, 1 equiv) at room temperature. Then NaBH(OAc)₃ (68 mg, 0.324 mmol, 2 equiv) was added at 0° C. after 0.5 h. The resulting mixture was stirred at room temperature for one hour. The resulting mixture was extracted with CH₂Cl₂ (2×3 mL). The combined organic layers were washed with brine (1×2 mL), and dried over anhydrous Na₂SO₄. After filtration, the filtrate was concentrated under reduced pressure. The residue was purified by reverse phase flash chromatography with MeCN:H₂O (1:1) to afford the desired product (21.3 mg, 15.98%) as a yellow solid. ¹H NMR (300 MHz, (CD₃)₂SO) δ 12.68 (s, 1H), 11.42 (s, 1H), 11.10 (s, 1H), 7.56 (d, J=8.1 Hz, 1H), 7.28 (s, 1H), 7.06-6.76 (m, 4H), 6.66-6.60 (m, 1H), 5.41-5.29 (m, 1H), 3.61 (s, 3H), 3.26-3.15 (m, 5H), 3.15-2.99 (m, 5H), 2.99-2.79 (m, 3H), 2.77-2.56 (m, 5H), 2.34-2.28 (m, 5H), 2.20-2.14 (m, 2H), 2.04-1.94 (m, 1H), 1.93-1.73 (m, 3H), 1.63-1.57 (m, 1H), 1.39 (d, J=2.4 Hz, 3H), 1.33-1.21 (m, 2H), 1.07-0.99 (m, 3H). LCMS: C₄₄H₅₂F₂N₁₀O₄ requires: 822, found: m/z=823 [M+H]⁺.

### Example 211. 5-(4-((4-((S)-1-((2-((4aS,5aR)-5,5-difluoro-5a-methyl-1,4,4a,5,5a,6-hexahydrocyclopropa[f]indazol-3-yl)-1H-indol-6-yl)(methyl)amino)-1-oxopropan-2-yl)piperazin-1-yl)methyl)piperidin-1-yl)-N-(2,6-dioxopiperidin-3-yl)picolinamide (66)

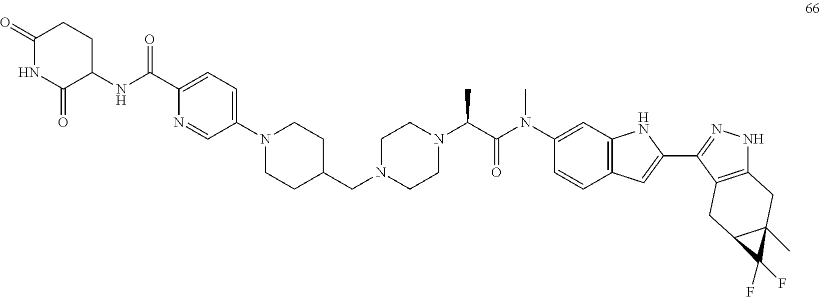 [SMILES: C[C@@H](C(=O)N(C)c1ccc2cc(-c3n[nH]c4c3C[C@@H]3C(F)(F)[C@]3(C)C4)[nH]c2c1)N1CCN(CC2CCN(c3ccc(C(=O)NC4CCC(=O)NC4=O)nc3)CC2)CC1]

To a mixture of (2S)—N-[2-[(4aS,5aR)-5,5-difluoro-5a-methyl-1H,4H,4aH,6H-cyclopropa[f]indazol-3-yl]-1H-indol-6-yl]-N-methyl-2-(piperazin-1-yl)propanamide (136 mg, 0.290 mmol, 1 equiv) in DCM (5 mL) was added DIEA (202 uL, 1.162 mmol, 4 equiv) and N-(2,6-dioxopiperidin-3-yl)-5-(4-formylpiperidin-1-yl)pyridine-2-carboxamide (100 mg, 0.290 mmol, 1 equiv) at room temperature. Then NaBH(OAc)₃ (123 mg, 0.581 mmol, 2 equiv) was added at 0° C. after 0.5 h. The resulting mixture was stirred at room temperature for one hour. The resulting mixture was extracted with CH₂Cl₂ (3×10 mL). The combined organic layers were washed with brine (1×5 mL) and dried over anhydrous Na₂SO₄. After filtration, the filtrate was concentrated under reduced pressure. The residue was purified by reverse phase flash chromatography with MeCN:H₂O (2:1) to afford the desired product (37.0 mg, 15.99%) as a white solid. ¹H NMR (300 MHz, (CD₃)₂SO) δ 12.76 (s, 1H), 11.53 (s, 1H), 10.86 (s, 1H), 9.76 (s, 1H), 8.71 (d, J=8.4 Hz, 1H), 8.36-8.29 (m, 1H), 7.86 (d, J=8.7 Hz, 1H), 7.64-7.55 (m, 1H), 7.47-7.38 (m, 1H), 7.34 (s, 1H), 6.98-6.92 (m, 1H), 6.65 (s, 1H), 4.82-4.67 (m, 1H), 4.00-3.90 (m, 2H), 3.51-3.35 (m, 3H), 3.23 (s, 3H), 3.16-2.65 (m, 13H), 2.55 (s, 1H), 2.30-1.94 (m, 3H), 1.92-1.82 (m, 3H), 1.45-0.97 (m, 9H). LCMS: C₄₂H₅₀F₂N₁₀O₄ requires: 796, found: m/z=797 [M+H]⁺

### Example 212. N-(2-((4aS,5aR)-5,5-difluoro-5a-methyl-1,4,4a,5,5a,6-hexahydrocyclopropa[f]indazol-3-yl)-1H-indol-6-yl)-3-((3R)-1-(2-(2,6-dioxopiperidin-3-yl)-1,3-dioxoisoindolin-5-yl)pyrrolidin-3-yl)-N-methylpropanamide (67)

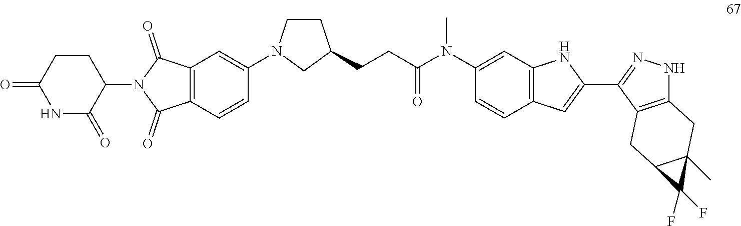 [SMILES: CN(C(=O)CC[C@@H]1CCN(c2ccc3c(c2)C(=O)N(C2CCC(=O)NC2=O)C3=O)C1)c1ccc2cc(-c3n[nH]c4c3C[C@@H]3C(F)(F)[C@]3(C)C4)[nH]c2c1]

A mixture of N-[2-[(4aS,5aR)-5,5-difluoro-5a-methyl-1-(oxan-2-yl)-4H,4aH,6H-cyclopropa[f]indazol-3-yl]-1H-indol-6-yl]-3-[(3R)-1-[2-(2,6-dioxopiperidin-3-yl)-1,3-dioxoisoindol-5-yl]pyrrolidin-3-yl]-N-methylpropanamide (140 mg, 0.176 mmol, 1 equiv) and HCl (4 M in dioxane, 8 mL) was stirred at room temperature for one hour. The resulting mixture was concentrated under vacuum. The crude product (60 mg) was purified by Prep-HPLC using the following conditions: Column: Xselect CSH OBD Column 30*150 mm 5 um; Mobile Phase A: Water (0.1% FA), Mobile Phase B: ACN; Flow rate: 60 mL/min; Gradient: 48% B to 56% B in 8 min, 56% B; Wavelength: 254/220 nm to afford the desired product (12 mg, 9.59%) as a yellow solid. ¹H NMR (300 MHz, (CD₃)₂SO) δ 11.80 (s, 1H), 10.59 (s, 1H), 9.85 (s, 1H), 7.76-7.60 (m, 1H), 7.59-7.50 (m, 1H), 7.50-7.41 (m, 1H), 7.05-6.92 (m, 1H), 6.92-6.78 (m, 1H), 6.78-6.69 (m, 1H), 6.68-6.55 (m, 1H), 5.11-4.83 (m, 1H), 3.55-3.43 (m, 2H), 3.43-3.32 (m, 1H), 3.32-3.26 (m, 3H), 3.26-3.10 (m, 3H), 3.01-2.85 (m, 3H), 2.79-2.71 (m, 3H), 2.31-2.20 (m, 3H), 1.90-1.74 (m, 3H), 1.68-1.55 (m, 1H), 1.49-1.39 (m, 3H), 1.32-1.25 (m, 1H). LCMS: C₃₈H₃₇F₂N₇O₅ requires: 709, found: m/z=710 [M+H]⁺.

### Example 213. N-(2-((4aS,5aR)-5,5-difluoro-5a-methyl-1,4,4a,5,5a,6-hexahydrocyclopropa[f]indazol-3-yl)-1H-indol-6-yl)-3-((3S)-1-(2-(2,6-dioxopiperidin-3-yl)-1,3-dioxoisoindolin-5-yl)pyrrolidin-3-yl −N-methylpropanamide (68)

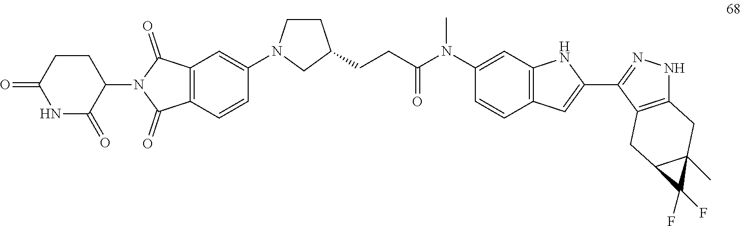 [SMILES: CN(C(=O)CC[C@H]1CCN(c2ccc3c(c2)C(=O)N(C2CCC(=O)NC2=O)C3=O)C1)c1ccc2cc(-c3n[nH]c4c3C[C@@H]3C(F)(F)[C@]3(C)C4)[nH]c2c1]

A mixture of N-[2-[(4aR,5aS)-5,5-difluoro-5a-methyl-1-(oxan-2-yl)-4H,4aH,6H-cyclopropa[f]indazol-3-yl]-1H-indol-6-yl]-3-[(3S)-1-[2-(2,6-dioxopiperidin-3-yl)-1,3-dioxoisoindol-5-yl]pyrrolidin-3-yl]-N-methylpropanamide (120 mg, 0.151 mmol, 1 equiv) and HCl (4 M in dioxane, 8 mL) was stirred for 2 h at room temperature. The resulting mixture was concentrated under vacuum. The crude product (90 mg) was purified by Prep-HPLC using the following conditions: Column: Xselect CSH OBD Column 30*150 mm Sum; Mobile Phase A: Water (0.1% FA), Mobile Phase B: ACN; Flow rate: 60 mL/min; Gradient: 46% B to 56% B in 9 min, 56% B; Wavelength: 254/220 nm to afford the desired product as a yellow solid. (21.2 mg, 18.12%). ¹H NMR (400 MHz, (CD₃)₂SO) δ 7.72-7.61 (m, 1H), 7.60-7.50 (m, 1H), 7.49-7.39 (m, 1H), 7.05-6.92 (m, 1H), 6.85-6.82 (m, 1H), 6.80-6.60 (m, 2H), 5.12-4.98 (m, 1H), 3.55-3.42 (m, 2H), 3.42-3.32 (m, 1H), 3.32-3.21 (m, 3H), 3.21-3.08 (m, 3H), 3.02-2.81 (m, 3H), 2.81-2.70 (m, 2H), 2.28-2.15 (m, 3H), 2.15-2.08 (m, 3H), 1.79-1.69 (m, 2H), 1.65-1.55 (m, 1H), 1.50-1.45 (m, 1H), 1.45-1.39 (m, 3H), 1.31-1.28 (m, 2H). LCMS: C₃₈H₃₇F₂N₇O₅ requires: 709, found: m/z=710 [M+H]⁺.

### Example 214. 5-(3-(3-((2-((4aS,5aR)-5,5-difluoro-5a-methyl-1,4,4a,5,5a,6-hexahydrocyclopropa[f]indazol-3-yl)-1H-indol-6-yl)(methyl)amino)-3-oxopropyl)pyrrolidin-1-yl)-N-(2,6-dioxopiperidin-3-yl)picolinamide (69)

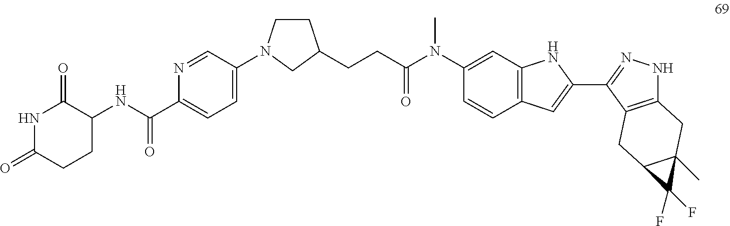 [SMILES: CN(C(=O)CCC1CCN(c2ccc(C(=O)NC3CCC(=O)NC3=O)nc2)C1)c1ccc2cc(-c3n[nH]c4c3C[C@@H]3C(F)(F)[C@]3(C)C4)[nH]c2c1]

To a mixture of 5-[3-[2-([2-[(4aS,5aR)-5,5-difluoro-5a-methyl-1-(oxan-2-yl)-4H,4aH,6H-cyclopropa[indazol-3-yl]-1H-indol-6-yl](methyl)carbamoyl)ethyl]pyrrolidin-1-yl]-N-(2,6-dioxopiperidin-3-yl)pyridine-2-carboxamide (100 mg, 0.130 mmol, 1 equiv) in DCM (8 mL) was added TFA (0.8 mL) at 0° C. The resulting mixture was stirred at 0° C. for 6 h. The resulting mixture was concentrated under vacuum. The crude product (100 mg) was purified by Prep-HPLC using the following conditions (Column: Xselect CSH OBD Column 30*150 mm Sum; Mobile Phase A: Water (10 mmoL/L NH₄HCO₃+0.1% NH₃H₂O), Mobile Phase B: MeCN; Flow rate: 60 mL/min; Gradient: 40% B to 50% B in 9 min, 50% B; Wavelength: 254/220 nm) to afford the desired product (7.6 mg, 8.32%) as a white solid. ¹H NMR (300 MHz, (CD₃)₂CO) δ 10.65 (s, 1H), 8.43 (d, J=7.5 Hz, 2H), 7.88-7.77 (m, 2H), 7.63 (d, J=9.0 Hz, 1H), 7.41 (s, 1H), 7-6.90 (m, 1H), 6.81 (d, J=6.0 Hz, 1H), 6.73 (s, 1H), 4.88-4.74 (m, 1H), 3.40 (m, 2H), 3.21-3.09 (m, 3H), 2.93-2.77 (m, 2H), 2.75-2.61 (m, 2H), 2.37-2.36 (m, 1H), 2.25-2.20 (m, 4H), 2.08-2.00 (m, 1H), 1.87-1.68 (m, 4H), 1.60-1.47 (m, 2H), 1.43 (s, 3H), 1.28 (s, 2H). LCMS: C₃₆H₃₈F₂N₈O₄ requires: 684, found: m/z=685 [M+H]⁺.

### Example 215. N-(2-((4aS,5aR)-5,5-difluoro-5a-methyl-1,4,4a,5,5a,6-hexahydrocyclopropa[f]indazol-3-yl)-1H-indol-6-yl)-3-(1-(5-(2,6-dioxopiperidin-3-yl)pyridin-2-yl)pyrrolidin-3-yl)-N-methylpropanamide (70)

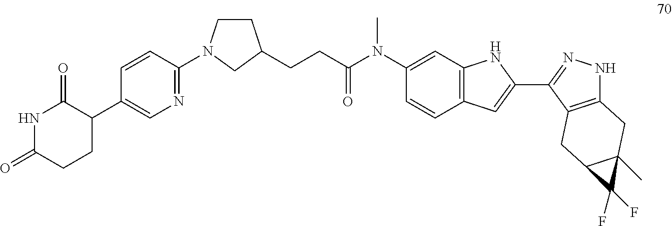 [SMILES: CN(C(=O)CCC1CCN(c2ccc(C3CCC(=O)NC3=O)cn2)C1)c1ccc2cc(-c3n[nH]c4c3C[C@@H]3C(F)(F)[C@]3(C)C4)[nH]c2c1]

A mixture of N-[2-[(4aS,5aR)-5,5-difluoro-5a-methyl-1-(oxan-2-yl)-4H,4aH,6H-cyclopropa[f]indazol-3-yl]-1H-indol-6-yl]-3-[1-[5-(2,6-dioxopiperidin-3-yl)pyridin-2-yl]pyrrolidin-3-yl]-N-methylpropanamide (25 mg, 0.034 mmol, 1 equiv) and HCl (4 M in dioxane, 3 mL) was stirred for one hour at room temperature. The resulting mixture was concentrated under vacuum to afford the desired product (2.3 mg, 4.52%) as a white solid. MS (ESI) calc'd for C₃₅H₃₇F₂N₇O₃[M+1]⁺: 642.2, found, 642.2. ¹HNMR (300 MHz, (CD₃)₂C₀) δ 10.83 (s, 1H), 9.58 (s, 1H), 7.97-7.91 (m, 1H), 7.68-7.59 (m, 1H), 7.48-7.35 (m, 2H), 7.01-6.91 (m, 1H), 6.76-6.69 (m, 1H), 6.42-6.33 (m, 1H), 3.82-3.71 (m, 1H), 3.53 (s, 2H), 3.40-3.29 (m, 1H), 3.26 (s, 3H), 3.24-3.10 (m, 4H), 2.77-2.56 (m, 3H), 2.26-2.14 (m, 6H), 1.84-1.68 (m, 2H), 1.60-1.44 (m, 1H), 1.48-1.40 (m, 3H), 1.30 (s, 1H). LCMS: C₃₅H₃₇F₂N₇O₃ requires: 641, found: m/z=642 [M+H]⁺.

### Example 216. (2r,4r)-N-[2-[(4aS,5aR)-5,5-difluoro-5a-methyl-1H,4H,4aH,6H-cyclopropa[f]indazol-3-yl]-1H-indol-6-yl]-6-[2-[(3RS)-2,6-dioxopiperidin-3-yl]-1,3-dioxoisoindol-5-yl]-N-methyl-6-azaspiro[3.4]octane-2-carboxamide (71a) and (2s,4s)-N-[2-[(4aS,5aR)-5,5-difluoro-5a-methyl-1H,4H,4aH,6H-cyclopropa[f]indazol-3-yl]-1H-indol-6-yl]-6-[2-[(3RS)-2,6-dioxopiperidin-3-yl]-1,3-dioxoisoindol-5-yl]-N-methyl-6-azaspiro[3.4]octane-2-carboxamide (71b)

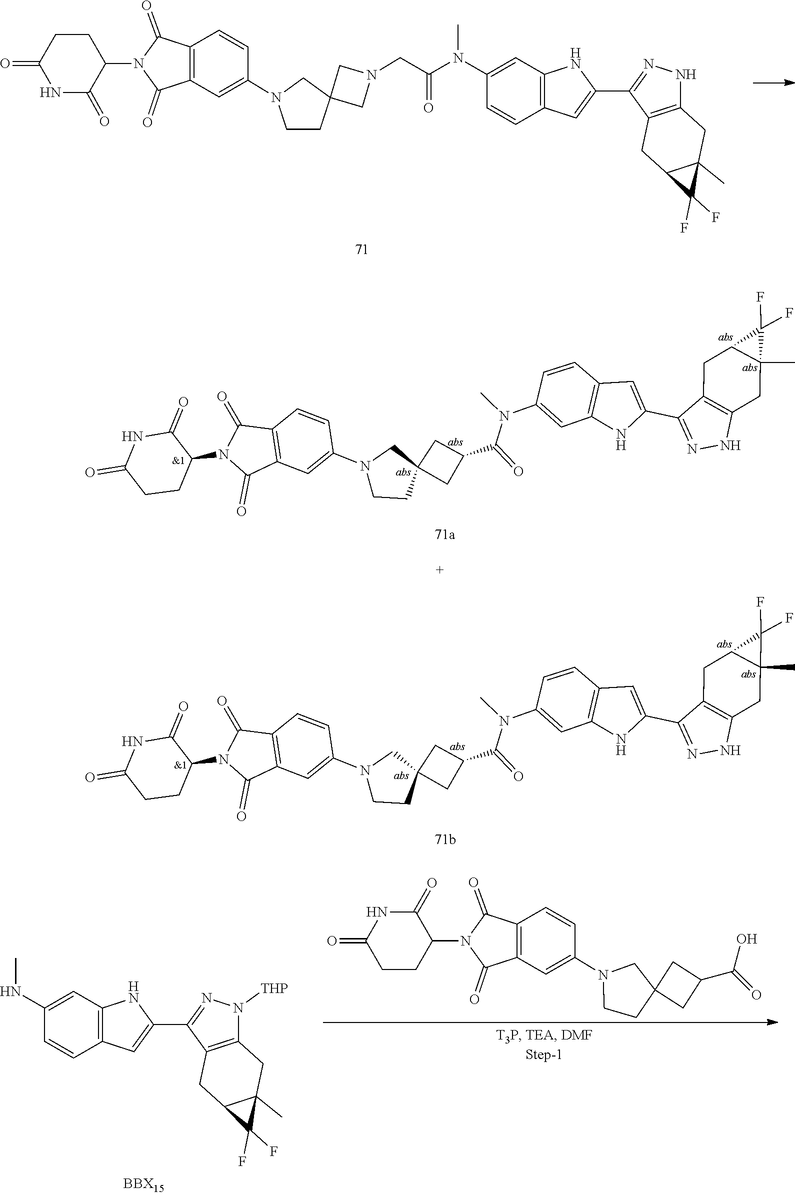

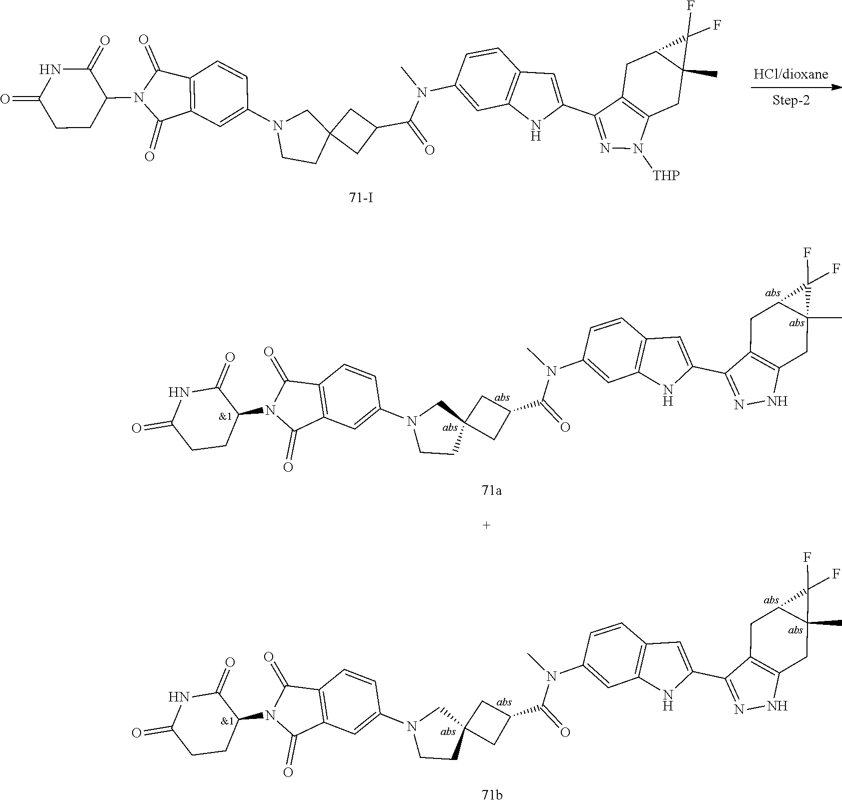

### Step-1: N-[2-[(4aS,5aR)-5,5-difluoro-5a-methyl-1-(oxan-2-yl)-4H,4aH,6H-cyclopropa[f]indazol-3-yl]-1H-indol-6-yl]-6-[2-(2,6-dioxopiperidin-3-yl)-1,3-dioxoisoindol-5-yl]-N-methyl-6-azaspiro[3.4]octane-2-carboxamide (71-I)

To a mixture of 6-[2-(2,6-dioxopiperidin-3-yl)-1,3-dioxoisoindol-5-yl]-6-azaspiro[3.4]octane-2-carboxylic acid (169 mg, 0.412 mmol, 1 equiv) in DMF (3 mL) is added TEA (250 mg, 2.473 mmol, 6 equiv). The resulting mixture was stirred at room temperature for 15 min under nitrogen atmosphere. To the above mixture is added 2-[(4aS,5aR)-5,5-difluoro-5a-methyl-1-(oxan-2-yl)-4H,4aH,6H-cyclopropa[f]indazol-3-yl]-N-methyl-1H-indol-6-amine (BBX₁₅) (170 mg, 0.412 mmol, 1 equiv) and T₃P (209 mg, 0.659 mmol, 1.5 equiv). The resulting mixture is stirred at room temperature overnight. The residue is purified using reverse phase column with ACN/H₂O (2/1) to afford N-[2-[(4aS,5aR)-5,5-difluoro-5a-methyl-1-(oxan-2-yl)-4H,4aH,6H-cyclopropa[f]indazol-3-yl]-1H-indol-6-yl]-6-[2-(2,6-dioxopiperidin-3-yl)-1,3-dioxoisoindol-5-yl]-N-methyl-6-azaspiro[3.4]octane-2-carboxamide (80 mg, 24.09% yield) as a brown solid. MS (ESI) calc'd for (C₄₄H₄₅F₂N₇O₆) [M+1]⁺, 806.3; found, 806.4

### Step-2: (2r,4r)-N-[2-[(4aS,5aR)-5,5-difluoro-5a-methyl-1H,4H,4aH,6H-cyclopropa[f]indazol-3-yl]-1H-indol-6-yl]-6-[2-[(3RS)-2,6-dioxopiperidin-3-yl]-1,3-dioxoisoindol-5-yl]-N-methyl-6-azaspiro[3.4]octane-2-carboxamide (71a) and (2s,4s)-N-[2-[(4aS,5aR)-5,5-difluoro-5a-methyl-1H,4H,4aH,6H-cyclopropa[f]indazol-3-yl]-1H-indol-6-yl]-6-[2-[(3RS)-2,6-dioxopiperidin-3-yl]-1,3-dioxoisoindol-5-yl]-N-methyl-6-azaspiro[3.4]octane-2-carboxamide (71b)

A mixture of N-[2-[(4aS,5aR)-5,5-difluoro-5a-methyl-1-(oxan-2-yl)-4H,4aH,6H-cyclopropa[f]indazol-3-yl]-1H-indol-6-yl]-6-[2-(2,6-dioxopiperidin-3-yl)-1,3-dioxoisoindol-5-yl]-N-methyl-6-azaspiro[3.4]octane-2-carboxamide (71b) (65 mg, 0.081 mmol, 1 equiv) in HCl/dioxane (2 mL, 4M) was stirred at 0° C. for 1 h. The resulting mixture was concentrated under vacuum. The crude product (40 mg) was purified by Prep-HPLC with the following conditions (Column, Xselect CSH OBD Column 30*150 mm 5 um; Mobile Phase A: Water (0.1% FA), Mobile Phase B: ACN; Flow rate: 60 mL/min; Gradient: 47 B to 55 B in 10 min; 254/220 nm) to afford (2r,4r)-N-[2-[(4aS,5aR)-5,5-difluoro-5a-methyl-1H,4H,4aH,6H-cyclopropa[f]indazol-3-yl]-1H-indol-6-yl]-6-[2-[(3RS)-2,6-dioxopiperidin-3-yl]-1,3-dioxoisoindol-5-yl]-N-methyl-6-azaspiro[3.4]octane-2-carboxamide (5.6 mg, 14.0% yield) as a yellow solid and (2s,4s)-N-[2-[(4aS,5aR)-5,5-difluoro-5a-methyl-1H,4H,4aH,6H-cyclopropa[f]indazol-3-yl]-1H-indol-6-yl]-6-[2-[(3RS)-2,6-dioxopiperidin-3-yl]-1,3-dioxoisoindol-5-yl]-N-methyl-6-azaspiro[3.4]octane-2-carboxamide (11.7 mg, 29.25% yield) as a yellow solid.

Compound 71a: MS (ESI) calc'd for (C₃₉H₃₇F₂N₇O₅) [M+1]⁺, 722.3; found, 722.3

Compound 71b: MS (ESI) calc'd for (C₃₉H₃₇F₂N₇O₅) [M+1]⁺, 722.3; found, 722.3

Compound 71a: ¹H NMR (300 MHz, CD₃COCD₃) δ 11.9 (s, 1H), δ 10.63 (s, 1H), 9.88 (s, 1H), 7.61 (d, J=8.4 Hz, 1H), 7.54 (d, J=8.4 Hz, 1H), 7.33 (d, J=1.8 Hz, 1H), 6.94-6.84 (m, 1H), 6.83-6.76 (m, 1H), 6.76-6.66 (m, 2H), 5.08-4.96 (m, 1H), 3.47-3.37 (m, 2H), 3.35-3.24 (m, 5H), 3.23-3.11 (m, 4H), 3.02-2.87 (m, 2H), 2.83-2.66 (m, 2H), 2.44-2.30 (m, 2H), 2.21-2.09 (m, 1H), 2.07-2.01 (m, 2H), 1.99-1.77 (m, 2H), 1.50-1.39 (m, 3H), 1.31 (s, 1H).

Compound 71b: ¹H NMR (300 MHz, CD₃COCD₃) δ 11.89 (s, 1H), 10.66 (s, 1H), 9.94 (s, 1H), 7.68-7.55 (m, 2H), 7.38-7.31 (m, 1H), 6.95-6.84 (m, 2H), 6.82-6.71 (m, 2H), 5.12-5.00 (m, 1H), 3.42-3.32 (m, 4H), 3.27 (s, 3H), 3.24-3.06 (m, 4H), 3.05-2.90 (m, 2H), 2.82-2.77 (m, 1H), 2.77-2.69 (m, 1H), 2.47-2.34 (m, 2H), 2.24-2.11 (m, 1H), 1.98-1.84 (m, 4H), 1.84-1.77 (m, 1H), 1.50-1.41 (m, 3H).

### Example 217. N-(2-((4aS,5aR)-5,5-difluoro-5a-methyl-1,4,4a,5,5a,6-hexahydrocyclopropa[f]indazol-3-yl)-1H-indol-6-yl)-2-(2-(2-(2,6-dioxopiperidin-3-yl)-1,3-dioxoisoindolin-5-yl)-2,8-diazaspiro[4.5]decan-8-yl)-N-methylacetamide (72)

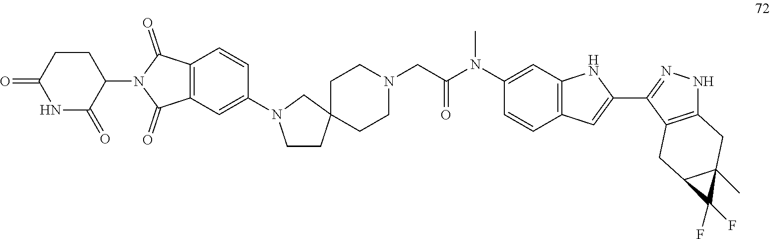 [SMILES: CN(C(=O)CN1CCC2(CC1)CCN(c1ccc3c(c1)C(=O)N(C1CCC(=O)NC1=O)C3=O)C2)c1ccc2cc(-c3n[nH]c4c3C[C@@H]3C(F)(F)[C@]3(C)C4)[nH]c2c1]

A mixture of N-[2-[(4aS,5aR)-5,5-difluoro-5a-methyl-1-(oxan-2-yl)-4H,4aH,6H-cyclopropa[f]indazol-3-yl]-1H-indol-6-yl]-2-[2-[2-(2,6-dioxopiperidin-3-yl)-1,3-dioxoisoindol-5-yl]-2,8-diazaspiro[4.5]decan-8-yl]-N-methylacetamide (85 mg, 0.1 mmol, 1 equiv) in HCl (4 M in dioxane, 5 mL) was stirred at 0° C. for 2 h. The resulting mixture was concentrated under vacuum. The crude product (80 mg) was purified by Prep-HPLC using the following conditions: Column, Xselect CSH OBD Column 30*150 mm 5 um; Mobile Phase A: Water (0.1% formic acid), Mobile Phase B: MeCN; Flow rate: 60 mL/min; Gradient: 25 B to 35 B in 7 min; 254/220 nm to afford the desired product as a yellow solid (12 mg, 15.67%). ¹H NMR (300 MHz, (CD₃)₂SO) δ 11.45 (s, 1H), 11.06 (s, 1H), 8.21 (s, 1H), 7.65-7.51 (m, 2H), 7.27 (s, 1H), 6.94-6.85 (m, 2H), 6.82-6.72 (m, 1H), 6.62 (s, 1H), 5.10-4.98 (m, 1H), 3.49-3.30 (m, 3H), 3.25-3.18 (m, 5H), 3.17-2.95 (m, 3H), 2.94-2.71 (m, 4H), 2.63-2.51 (m, 2H), 2.46-2.39 (m, 1H), 2.35-2.25 (m, 2H), 2.05-1.94 (m, 1H), 1.92-1.72 (m, 3H), 1.54-1.45 (m, 4H), 1.38 (s, 3H). LCMS: C₄₁H₄₂F₂N₈O₅ requires 764, found: m/z=765 [M+H]⁺.

### Example 218. N-(2-((4aS,5aR)-5,5-difluoro-5a-methyl-1,4,4a,5,5a,6-hexahydrocyclopropa[f]indazol-3-yl)-1H-indol-6-yl)-2-(1-((1-(5-(2,6-dioxopiperidin-3-yl)pyridin-2-yl)piperidin-4-yl)methyl)piperidin-4-yl)-N-methylacetamide (73)

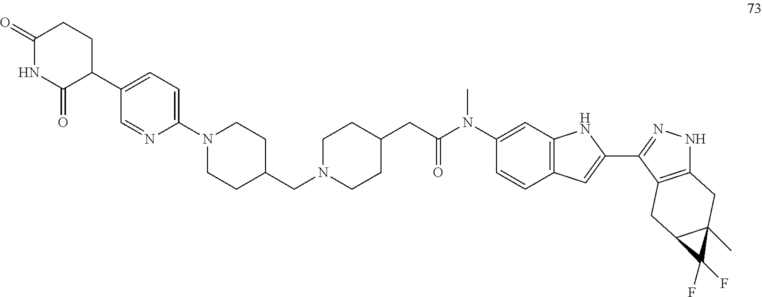 [SMILES: CN(C(=O)CC1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1]

A mixture of N-{2-[(4aS,5aR)-5,5-difluoro-5a-methyl-1H,4H,4aH,6H-cyclopropa[f]indazol-3-yl]-1H-indol-6-yl}-N-methyl-2-(piperidin-4-yl)acetamide (20.50 mg, 0.05 mmol) and N,N-diisopropylethylamine (32.00 uL, 0.02 g, 0.18 mmol) in DCM (1.00 mL) was added to rac-1-{5-[(3R)-2,6-dioxopiperidin-3-yl]pyridin-2-yl}piperidine-4-carbaldehyde (15.50 mg, 0.05 mmol) followed by sodium triacetoxyborohydride (19.16 mg, 0.09 mmol). After 30 min, the reaction was diluted with DCM, concentrated, and purified by flash chromatography on a 4 g column, eluting by gradient elution with 0 to 20% MeOH/DCM to provide N-{2-[(4aS,5aR)-5,5-difluoro-5a-methyl-1H,4H,4aH,6H-cyclopropa[f]indazol-3-yl]-1H-indol-6-yl}-2-{1-[(1-{5-[(3RS)-2,6-dioxopiperidin-3-yl]pyridin-2-yl}piperidin-4-yl)methyl]piperidin-4-yl}-N-methylacetamide (0.017 g, 42.1%) as a white solid. ¹H NMR (500 MHz, (CD₃)₂SO) δ 12.68 (s, 1H), 11.42 (s, 1H), 10.78 (s, 1H), 7.91 (d, J=2.5 Hz, 1H), 7.57 (d, J=8.2 Hz, 1H), 7.33 (dd, J=8.8, 2.5 Hz, 1H), 7.22 (s, 1H), 6.85 (d, J=8.2 Hz, 1H), 6.74 (d, J=8.8 Hz, 1H), 6.63 (s, 1H), 4.20 (d, J=12.7 Hz, 2H), 3.70 (dd, J=12.1, 4.9 Hz, 1H), 3.18 (s, 3H), 3.12 (t, J=8.0 Hz, 1H), 3.08-2.97 (m, 2H), 2.87-2.61 (m, 7H), 2.14 (tt, J=12.5, 6.4 Hz, 3H), 1.96 (dd, J=11.5, 5.8 Hz, 3H), 1.86 (dd, J=14.8, 7.0 Hz, 1H), 1.68 (d, J=12.6 Hz, 5H), 1.56 (d, J=12.5 Hz, 2H), 1.39 (s, 3H), 0.99 (p, J=12.8 Hz, 5H). LCMS: C₄₁H₄₈F₂N₈O₃ requires: 738.4, found: m/z=739.6 [M+H]⁺.

### Example 219. N-(2-((4aS,5aR)-5,5-difluoro-5a-methyl-1,4,4a,5,5a,6-hexahydrocyclopropa[f]indazol-3-yl)-1H-indol-6-yl)-1-((1-(5-(2,6-dioxopiperidin-3-yl)pyridin-2-yl)piperidin-4-yl)methyl)-N-methylpiperidine-4-carboxamide (74)

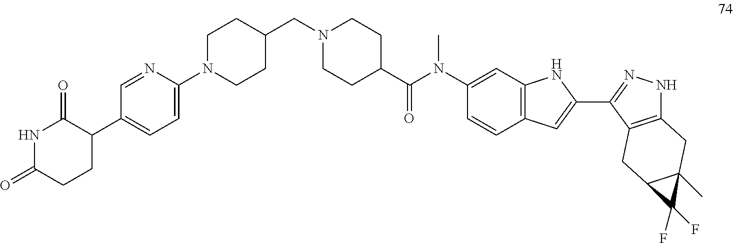 [SMILES: CN(C(=O)C1CCN(CC2CCN(c3ccc(C4CCC(=O)NC4=O)cn3)CC2)CC1)c1ccc2cc(-c3n[nH]c4c3C[C@@H]3C(F)(F)[C@]3(C)C4)[nH]c2c1]

A mixture of N-{2-[(4aS,5aR)-5,5-difluoro-5a-methyl-1H,4H,4aH,6H-cyclopropa[f]indazol-3-yl]-1H-indol-6-yl}-N-methylpiperidine-4-carboxamide (22.38 mg, 0.05 mmol) and N,N-diisopropylethylamine (35.00 uL, 0.03 g, 0.20 mmol) in DCM (1.00 mL) was added to rac-1-{5-[(3R)-2,6-dioxopiperidin-3-yl]pyridin-2-yl}piperidine-4-carbaldehyde (15.34 mg, 0.05 mmol) followed by sodium triacetoxyborohydride (21.58 mg, 0.10 mmol). After 1 h, the reaction was quenched with water. The mixture was extracted twice with DCM. The combined organic layers were concentrated and purified by flash chromatography on a 4 g column, eluting by gradient elution with 0 to 20% MeOH/DCM to provide N-{2-[(4aS,5aR)-5,5-difluoro-5a-methyl-1H,4H,4aH,6H-cyclopropa[f]indazol-3-yl]-1H-indol-6-yl}-1-[(1-{5-[(3RS)-2,6-dioxopiperidin-3-yl]pyridin-2-yl}piperidin-4-yl)methyl]-N-methylpiperidine-4-carboxamide (0.0032 g, 6.0%) as a white solid. ¹H NMR (500 MHz, (CD₃)₂SO) δ 12.68 (s, 1H), 11.41 (s, 1H), 10.78 (s, 1H), 7.91 (d, J=2.4 Hz, 1H), 7.58 (d, J=8.5 Hz, 1H), 7.47 (d, J=7.9 Hz, 1H), 7.36-7.31 (m, 1H), 7.25 (s, 1H), 7.11 (d, J=7.8 Hz, 1H), 6.89 (d, J=8.4 Hz, 1H), 6.75 (d, J=8.8 Hz, 1H), 6.63 (s, 1H), 4.21 (d, J=12.7 Hz, 2H), 3.70 (dd, J=12.2, 4.9 Hz, 1H), 3.17 (s, 4H), 3.08-2.97 (m, 2H), 2.84 (d, J=17.2 Hz, 1H), 2.76-2.62 (m, 3H), 2.29 (s, 1H), 2.17 (dd, J=12.7, 8.5 Hz, 1H), 2.01-1.92 (m, 1H), 1.86 (d, J=9.3 Hz, 1H), 1.67 (d, J=12.5 Hz, 5H), 1.55 (br s, 3H), 1.39 (s, 3H), 1.23 (s, 1H), 1.22-1.14 (m, 1H), 1.08-0.95 (m, 2H), 0.90-0.76 (m, 1H). LCMS: C₄₀H₄₆F₂N₈O₃ requires: 724.4, found: m/z=725.6 [M+H]⁺.

### Example 220. 5-(4-((4-(2-((2-((4aS,5aR)-5,5-difluoro-5a-methyl-1,4,4a,5,5a,6-hexahydrocyclopropa[f]indazol-3-yl)-1H-indol-6-yl)(methyl)amino)-2-oxoethyl)piperidin-1-yl)methyl)piperidin-1-yl)-N-(2,6-dioxopiperidin-3-yl)picolinamide (75)

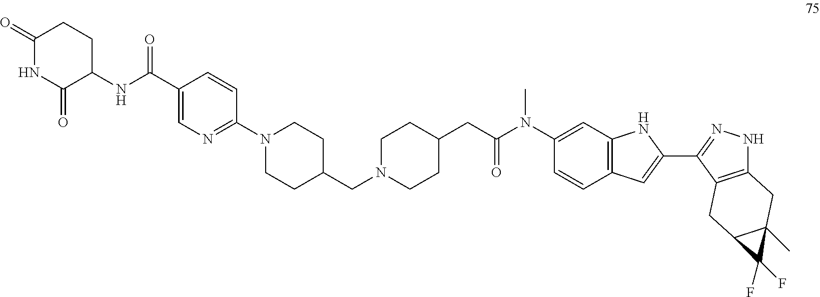 [SMILES: CN(C(=O)CC1CCN(CC2CCN(c3ccc(C(=O)NC4CCC(=O)NC4=O)cn3)CC2)CC1)c1ccc2cc(-c3n[nH]c4c3C[C@@H]3C(F)(F)[C@]3(C)C4)[nH]c2c1]

A mixture of N-{2-[(4aS,5aR)-5,5-difluoro-5a-methyl-1H,4H,4aH,6H-cyclopropa[f]indazol-3-yl]-1H-indol-6-yl}-N-methyl-2-(piperidin-4-yl)acetamide (32.08 mg, 0.07 mmol) and N,N-diisopropylethylamine (48.00 uL, 0.04 g, 0.28 mmol) in DCM (1.50 mL) was added to rac-N-[(3R)-2,6-dioxopiperidin-3-yl]-5-(4-formylpiperidin-1-yl)pyridine-2-carboxamide (25.00 mg, 0.07 mmol) followed by sodium triacetoxyborohydride (30.77 mg, 0.15 mmol). After 1 h, the reaction was quenched with water. The mixture was extracted twice with DCM. The combined organic layers were concentrated and purified by flash chromatography on a 4 g column, eluting by gradient elution with 0 to 20% MeOH/DCM to provide 5-[4-({4-[({2-[(4aS,5aR)-5,5-difluoro-5a-methyl-1H,4H,4aH,6H-cyclopropa[f]indazol-3-yl]-1H-indol-6-yl}(methyl)carbamoyl)methyl]piperidin-1-yl}methyl)piperidin-1-yl]-N-[(3RS)-2,6-dioxopiperidin-3-yl]pyridine-2-carboxamide (0.0210 g, 36.4%) as a white solid. ¹H NMR (500 MHz, (CD₃)₂SO) δ 12.67 (s, 1H), 11.42 (s, 1H), 10.83 (s, 1H), 8.68 (d, J=8.3 Hz, 1H), 8.27 (d, J=2.8 Hz, 1H), 7.82 (d, J=8.8 Hz, 1H), 7.57 (d, J=8.3 Hz, 1H), 7.36 (d, J=8.9 Hz, 1H), 7.21 (s, 1H), 6.85 (d, J=8.3 Hz, 1H), 6.63 (s, 1H), 4.73 (dt, J=13.1, 6.9 Hz, 1H), 4.09 (q, J=5.3 Hz, 2H), 3.89 (d, J=12.6 Hz, 2H), 3.18 (s, 3H), 3.11 (d, J=6.3 Hz, 1H), 3.08-2.98 (m, 2H), 2.89-2.68 (m, 5H), 2.16 (qd, J=12.9, 4.3 Hz, 1H), 2.08 (d, J=6.2 Hz, 1H), 2.04-1.97 (m, 1H), 1.95 (d, J=6.5 Hz, 2H), 1.91 (s, 2H), 1.86 (dd, J=15.3, 6.8 Hz, 1H), 1.73 (d, J=12.5 Hz, 4H), 1.56 (s, 2H), 1.39 (s, 3H), 1.12 (d, J=12.2 Hz, 2H), 0.97 (s, 2H). LCMS: C₄₂H₄₉F₂N₉O₄ requires: 781.4, found: m/z=782.6 [M+H]⁺.

### Example 221. (4aS,5aR)-N-(1-(1-((1-(4-(2,6-dioxopiperidin-3-yl)phenyl)piperidin-4-yl)methyl)piperidin-4-yl)-1H-pyrazol-4-yl)-5,5-difluoro-5a-methyl-1,4,4a,5,5a,6-hexahydrocyclopropa[f]indazole-3-carboxamide (92)

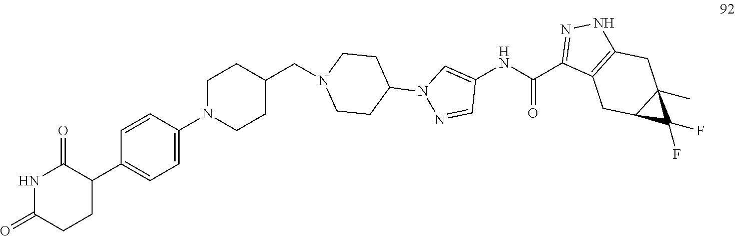 [SMILES: C[C@@]12Cc3[nH]nc(C(=O)Nc4cnn(C5CCN(CC6CCN(c7ccc(C8CCC(=O)NC8=O)cc7)CC6)CC5)c4)c3C[C@@H]1C2(F)F]

Using general procedure 15, (4aS,5aR)-5,5-difluoro-5a-methyl-N-(1-(piperidin-4-yl)-1H-pyrazol-4-yl)-1,4,4a,5,5a,6-hexahydrocyclopropa[f]indazole-3-carboxamide (30 mg, 0.080 mmol) was treated with 1-(4-(2,6-dioxopiperidin-3-yl)phenyl)piperidine-4-carbaldehyde (HCB64) (26 mg, 0.088 mmol) to afford the title compound (16 mg, 0.024 mmol, 30%). ¹H NMR (500 MHz, (CD₃)₂SO) δ 12.96 (s, 1H), 10.77 (s, 1H), 10.12 (s, 1H), 8.01 (s, 1H), 7.64 (s, 1H), 7.04 (d, J=8.0 Hz, 2H), 6.90 (d, J=8.1 Hz, 2H), 4.09 (s, 1H), 3.73 (dd, J=10.7, 4.6 Hz, 1H), 3.67 (d, J=12.0 Hz, 2H), 3.07 (d, J=9.6 Hz, 2H), 3.03 (s, 1H), 2.94 (d, J=10.9 Hz, 2H), 2.83 (d, J=17.1 Hz, 1H), 2.66 (d, J=12.1 Hz, 2H), 2.62 (s, 1H), 2.47 (d, J=21.0 Hz, 2H), 2.21 (s, 1H), 2.17-1.85 (m, 5H), 1.81 (d, J=11.8 Hz, 3H), 1.67 (s, 1H), 1.36 (s, 3H), 1.22 (q, J=12.5, 11.3 Hz, 2H). LCMS: C₃₅H₄₂F₂N₈O₃ requires: 660, found: m/z=661 [M+H]⁺.

### Example 222. (4aS,5aR)-N-(1-((3S,4S)-1-((1-(6-(((S)-2,6-dioxopiperidin-3-yl)carbamoyl)pyridin-3-yl)piperidin-4-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (93)

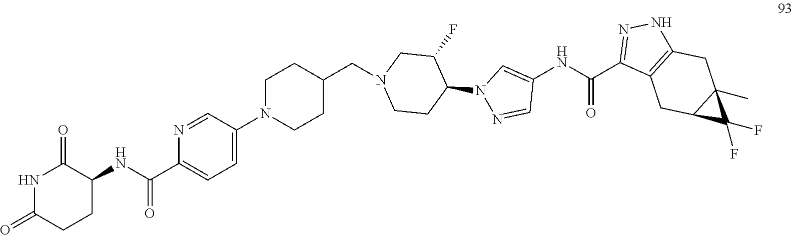 [SMILES: C[C@@]12Cc3[nH]nc(C(=O)Nc4cnn([C@H]5CCN(CC6CCN(c7ccc(C(=O)N[C@H]8CCC(=O)NC8=O)nc7)CC6)C[C@@H]5F)c4)c3C[C@@H]1C2(F)F]

Using general procedure 15, (4aS,5aR)-5,5-difluoro-N-(1-((3S,4S)-3-fluoropiperidin-4-yl)-1H-pyrazol-4-yl)-5a-methyl-1,4,4a,5,5a,6-hexahydrocyclopropa[f]indazole-3-carboxamide (32 mg, 0.081 mmol) was treated with (S)—N-(2,6-dioxopiperidin-3-yl)-5-(4-formylpiperidin-1-yl)picolinamide (HCB28) (31 mg, 0.089 mmol) to afford the title compound (22 mg, 0.030 mmol, 36%). LCMS: C₃₅H₄₁F₃N₁₀O₄ requires: 722, found: m/z=723 [M+H]⁺.

### Example 223. (4aS,5aR)-N-(1-((3R,4R)-1-((1-(6-(((S)-2,6-dioxopiperidin-3-yl)carbamoyl)pyridin-3-yl)piperidin-4-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (94)

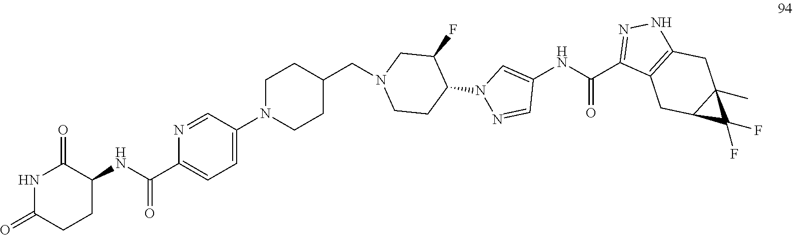 [SMILES: C[C@@]12Cc3[nH]nc(C(=O)Nc4cnn([C@@H]5CCN(CC6CCN(c7ccc(C(=O)N[C@H]8CCC(=O)NC8=O)nc7)CC6)C[C@H]5F)c4)c3C[C@@H]1C2(F)F]

Using general procedure 15, (4aS,5aR)-5,5-difluoro-N-(1-((3R,4R)-3-fluoropiperidin-4-yl)-1H-pyrazol-4-yl)-5a-methyl-1,4,4a,5,5a,6-hexahydrocyclopropa[f]indazole-3-carboxamide (30 mg, 0.076 mmol) was treated with (S)—N-(2,6-dioxopiperidin-3-yl)-5-(4-formylpiperidin-1-yl)picolinamide (HCB28) (29 mg, 0.084 mmol) to afford the title compound (27 mg, 0.036 mmol, 47%). LCMS: C₃₅H₄₁F₃N₁₀O₄ requires: 722, found: m/z=723 [M+H]⁺.

### Example 224. (4aS,5aR)-N-(1-((3S,4S)-1-((1-(4-((R)-2,6-dioxopiperidin-3-yl)phenyl)piperidin-4-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (*arbitrarily assigned)

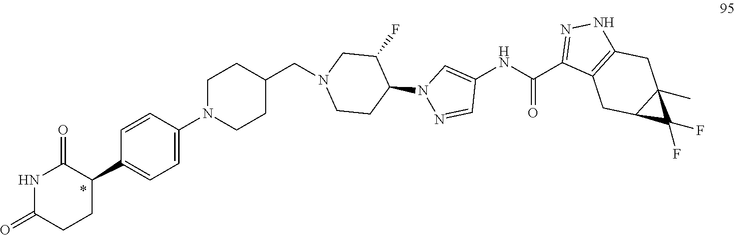 [SMILES: C[C@@]12Cc3[nH]nc(C(=O)Nc4cnn([C@H]5CCN(CC6CCN(c7ccc([C@H]8CCC(=O)NC8=O)cc7)CC6)C[C@@H]5F)c4)c3C[C@@H]1C2(F)F]

Using general procedure 15, (4aS,5aR)-5,5-difluoro-N-(1-((3S,4S)-3-fluoropiperidin-4-yl)-1H-pyrazol-4-yl)-5a-methyl-1,4,4a,5,5a,6-hexahydrocyclopropa[f]indazole-3-carboxamide (150 mg, 0.38 mmol) was treated with (R)-1-(4-(2,6-dioxopiperidin-3-yl)phenyl)piperidine-4-carbaldehyde (HCB64) (114 mg, 0.38 mmol) to afford the title compound (30 mg, 0.044 mmol, 12%). ¹H NMR (400 MHz, (CD₃)₂SO) δ 12.97 (s, 1H), 10.78 (s, 1H), 10.17 (s, 1H), 8.09 (s, 1H), 7.70 (s, 1H), 7.02-7.05 (m, 2H), 6.90-6.88 (m, 2H), 4.93-4.70 (m, 1H), 4.39-4.20 (m, 1H), 3.74-3.65 (m, 3H), 3.33-3.20 (m, 1H), 3.20-3.12 (m, 2H), 3.10-3.01 (m, 1H), 2.92-2.80 (m, 2H), 2.70-2.60 (m, 3H), 2.48-2.44 (m, 1H), 2.30-2.20 (m, 2H), 2.08-2.02 (m, 4H), 2.01-1.90 (m, 2H), 1.82-1.78 (m, 3H), 1.72-1.62 (m, 1H), 1.38-1.35 (m, 3H), 1.22-1.10 (m, 2H). LCMS: C₃₅H₄₁F₃N₈O₃ requires: 678, found: m/z=679 [M+H]⁺.

### Example 225. (4aS,5aR)-N-(1-((3R,4R)-1-((1-(4-((R)-2,6-dioxopiperidin-3-yl)phenyl)piperidin-4-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (96) (*Arbitrarily Assigned)

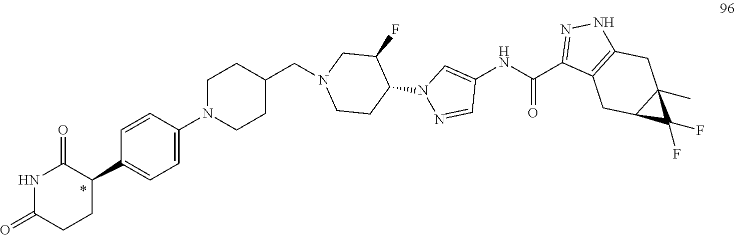 [SMILES: C[C@@]12Cc3[nH]nc(C(=O)Nc4cnn([C@@H]5CCN(CC6CCN(c7ccc([C@H]8CCC(=O)NC8=O)cc7)CC6)C[C@H]5F)c4)c3C[C@@H]1C2(F)F]

Using general procedure 15, (4aS,5aR)-5,5-difluoro-N-(1-((3R,4R)-3-fluoropiperidin-4-yl)-1H-pyrazol-4-yl)-5a-methyl-1,4,4a,5,5a,6-hexahydrocyclopropa[f]indazole-3-carboxamide (100 mg, 0.25 mmol) was treated with (R)-1-(4-(2,6-dioxopiperidin-3-yl)phenyl)piperidine-4-carbaldehyde (HCB64) (76 mg, 0.25 mmol) to afford the title compound (22 mg, 0.032 mmol, 13%). ¹H NMR (400 MHz, (CD₃)₂SO) δ 12.97 (s, 1H), 10.77 (s, 1H), 10.16 (s, 1H), 8.09 (s, 1H), 7.71 (s, 1H), 7.04 (d, J=8.4 Hz, 2H), 6.95-6.75 (m, 2H), 4.90-4.73 (m, 1H), 4.39-4.21 (m, 1H), 3.74-3.65 (m, 3H), 3.32-3.28 (m, 1H), 3.13-3.02 (m, 3H), 2.96-2.85 (m, 2H), 2.67-2.60 (m, 3H), 2.50-2.44 (m, 1H), 2.35-2.29 (m, 2H), 2.20-1.97 (m, 6H), 1.98-1.80 (m, 3H), 1.78-1.60 (m, 1H), 1.36 (s, 3H), 1.32-1.20 (m, 2H). LCMS: C₃₅H₄₁F₃N₈O₃ requires: 678, found: m/z=679 [M+H]⁺.

### Example 226. (4aS,5aR)-N-(1-(1-((1-(4-(2,6-dioxopiperidin-3-yl)phenyl)-4-fluoropiperidin-4-yl)methyl)piperidin-4-yl)-1H-pyrazol-4-yl)-5,5-difluoro-5a-methyl-1,4,4a,5,5a,6-hexahydrocyclopropa[f]indazole-3-carboxamide (97)

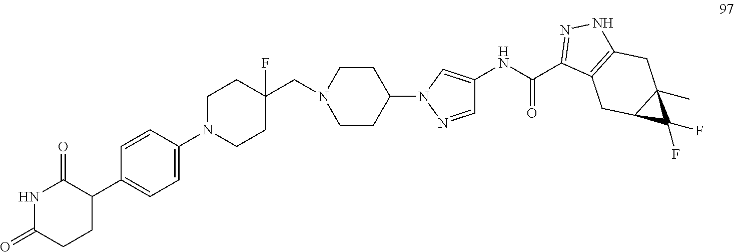 [SMILES: C[C@@]12Cc3[nH]nc(C(=O)Nc4cnn(C5CCN(CC6(F)CCN(c7ccc(C8CCC(=O)NC8=O)cc7)CC6)CC5)c4)c3C[C@@H]1C2(F)F]

Using general procedure 15, (4aS,5aR)-5,5-difluoro-5a-methyl-N-(1-(piperidin-4-yl)-1H-pyrazol-4-yl)-1,4,4a,5,5a,6-hexahydrocyclopropa[f]indazole-3-carboxamide (30 mg, 0.080 mmol) was treated with 1-(4-(2,6-dioxopiperidin-3-yl)phenyl)-4-fluoropiperidine-4-carbaldehyde (26 mg, 0.088 mmol) to afford the title compound (35 mg, 0.051 mmol, 64%). ¹H NMR (500 MHz, (CD₃)₂SO) δ 12.97 (s, 1H), 10.78 (s, 1H), 10.12 (s, 1H), 8.02 (s, 1H), 7.64 (s, 1H), 7.06 (d, J=8.3 Hz, 2H), 6.94 (d, J=8.4 Hz, 2H), 4.08 (p, J=7.9 Hz, 1H), 3.74 (dd, J=11.0, 4.9 Hz, 1H), 3.47 (dt, J=13.1, 4.2 Hz, 2H), 3.07 (d, J=9.8 Hz, 2H), 3.00 (t, J=11.6 Hz, 5H), 2.83 (d, J=15.8 Hz, 1H), 2.69-2.60 (m, 2H), 2.57 (s, 1H), 2.51-2.43 (m, 1H), 2.34-2.25 (m, 2H), 2.14 (qd, J=11.5, 4.4 Hz, 1H), 2.02 (dq, J=13.5, 5.0 Hz, 1H), 1.93 (dt, J=9.8, 5.7 Hz, 6H), 1.87-1.71 (m, 3H), 1.36 (s, 3H). LCMS: C₃₅H₄₁F₃N₈O₃ requires: 679, found: m/z=680 [M+H]⁺.

### Example 227. (4aS,5aR)-N-(1-((3S,4S)-1-(((3R)-1-(4-(2,6-dioxopiperidin-3-yl)phenyl)pyrrolidin-3-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (98)

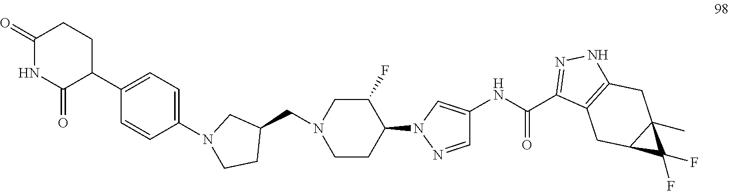 [SMILES: C[C@@]12Cc3[nH]nc(C(=O)Nc4cnn([C@H]5CCN(C[C@H]6CCN(c7ccc(C8CCC(=O)NC8=O)cc7)C6)C[C@@H]5F)c4)c3C[C@@H]1C2(F)F]

Using general procedure 15, (4aS,5aR)-5,5-difluoro-N-(1-((3S,4S)-3-fluoropiperidin-4-yl)-1H-pyrazol-4-yl)-5a-methyl-1,4,4a,5,5a,6-hexahydrocyclopropa[f]indazole-3-carboxamide (30 mg, 0.076 mmol) was treated with (3S)-1-(4-(2,6-dioxopiperidin-3-yl)phenyl)pyrrolidine-3-carbaldehyde (24 mg, 0.084 mmol) to afford the title compound (27 mg, 0.041 mmol, 53%). ¹H NMR (500 MHz, (CD₃)₂SO) δ 12.96 (s, 1H), 10.74 (s, 1H), 10.16 (s, 1H), 8.09 (s, 1H), 7.70 (s, 1H), 6.99 (d, J=8.1 Hz, 2H), 6.50 (d, J=8.1 Hz, 2H), 4.84 (d, J=50.2 Hz, 1H), 4.29 (s, 1H), 3.68 (dd, J=10.7, 5.0 Hz, 1H), 3.38 (s, 1H), 3.21 (d, J=8.2 Hz, 1H), 3.12-2.88 (m, 5H), 2.86-2.78 (m, 1H), 2.61 (ddt, J=19.4, 13.8, 6.8 Hz, 2H), 2.44 (dt, J=17.3, 4.7 Hz, 3H), 2.20-1.91 (m, 3H), 1.78 (dd, J=15.5, 5.7 Hz, 1H), 1.71 (dd, J=12.5, 7.6 Hz, 1H), 1.35 (s, 3H). LCMS: C₃₄H₃₉F₃N₈O₃ requires: 665, found: m/z=666 [M+H]⁺.

### Example 228. (4aS,5aR)-N-(1-((3R,4R)-1-(((3R)-1-(4-(2,6-dioxopiperidin-3-yl)phenyl)pyrrolidin-3-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (99)

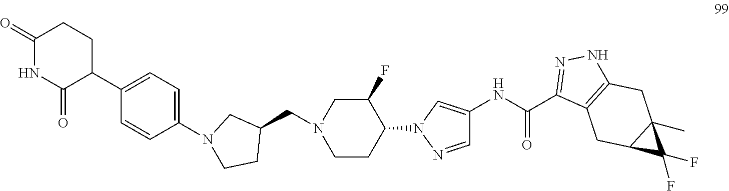 [SMILES: C[C@@]12Cc3[nH]nc(C(=O)Nc4cnn([C@@H]5CCN(C[C@H]6CCN(c7ccc(C8CCC(=O)NC8=O)cc7)C6)C[C@H]5F)c4)c3C[C@@H]1C2(F)F]

Using general procedure 15, (4aS,5aR)-5,5-difluoro-N-(1-((3R,4R)-3-fluoropiperidin-4-yl)-1H-pyrazol-4-yl)-5a-methyl-1,4,4a,5,5a,6-hexahydrocyclopropa[f]indazole-3-carboxamide (30 mg, 0.076 mmol) was treated with (3S)-1-(4-(2,6-dioxopiperidin-3-yl)phenyl)pyrrolidine-3-carbaldehyde (24 mg, 0.084 mmol) to afford the title compound (26 mg, 0.038 mmol, 49%). ¹HNMR (500 MHz, (CD₃)₂SO) δ 12.96 (s, 1H), 10.74 (s, 1H), 10.15 (s, 1H), 8.08 (s, 1H), 7.70 (s, 1H), 6.99 (d, J=8.2 Hz, 2H), 6.49 (d, J=8.2 Hz, 2H), 5.75 (s, 1H), 4.94-4.72 (m, 1H), 4.35-4.24 (m, 1H), 4.09 (q, J=5.3 Hz, 1H), 3.68 (dd, J=10.6, 5.0 Hz, 1H), 3.19 (dd, J=21.2, 6.1 Hz, 2H), 3.13-2.94 (m, 5H), 2.86-2.78 (m, 1H), 2.61 (ddt, J=19.4, 13.8, 6.6 Hz, 2H), 2.49-2.40 (m, 3H), 2.21-1.92 (m, 5H), 1.78 (dd, J=15.3, 5.9 Hz, 1H), 1.70 (dt, J=11.8, 7.6 Hz, 1H), 1.35 (s, 3H). LCMS: C₃₄H₃₉F₃N₈O₃ requires: 665, found: m/z=666 [M+H]⁺.

### Example 229. (4aS,5aR)-N-(1-((3S,4S)-1-(((3S)-1-(4-(2,6-dioxopiperidin-3-yl)phenyl)pyrrolidin-3-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (100)

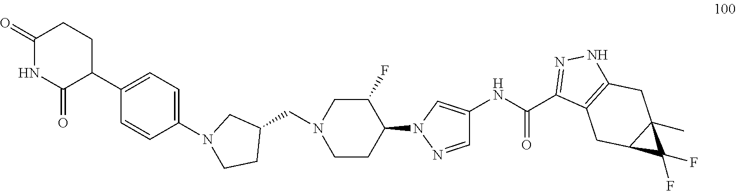 [SMILES: C[C@@]12Cc3[nH]nc(C(=O)Nc4cnn([C@H]5CCN(C[C@@H]6CCN(c7ccc(C8CCC(=O)NC8=O)cc7)C6)C[C@@H]5F)c4)c3C[C@@H]1C2(F)F]

Using general procedure 15, (4aS,5aR)-5,5-difluoro-N-(1-((3S,4S)-3-fluoropiperidin-4-yl)-1H-pyrazol-4-yl)-5a-methyl-1,4,4a,5,5a,6-hexahydrocyclopropa[f]indazole-3-carboxamide (15 mg, 0.038 mmol) was treated with (3R)-1-(4-(2,6-dioxopiperidin-3-yl)phenyl)pyrrolidine-3-carbaldehyde (12 mg, 0.042 mmol) to afford the title compound (16 mg, 0.024 mmol, 62%). ¹H NMR (500 MHz, (CD₃)₂SO) δ 12.98 (s, 1H), 10.75 (s, 1H), 10.17 (s, 1H), 8.10 (s, 1H), 7.71 (s, 1H), 7.00 (d, J=8.2 Hz, 2H), 6.51 (d, J=8.2 Hz, 2H), 5.77 (s, 3H), 4.86 (ddt, J=50.3, 10.0, 5.3 Hz, 1H), 4.34-4.25 (m, 1H), 4.10 (q, J=5.2 Hz, OH), 3.69 (dd, J=10.6, 5.0 Hz, 1H), 3.39 (dd, J=10.0, 7.0 Hz, 1H), 3.20 (dd, J=20.4, 6.6 Hz, 2H), 3.14-2.89 (m, 5H), 2.83 (d, J=16.6 Hz, 1H), 2.62 (ddd, J=24.8, 12.4, 6.6 Hz, 2H), 2.50-2.41 (m, 2H), 2.23-1.92 (m, 3H), 1.79 (dd, J=15.5, 5.9 Hz, 1H), 1.72 (dq, J=14.9, 7.7 Hz, 1H), 1.37 (s, 3H). LCMS: C₃₄H₃₉F₃N₈O₃ requires: 665, found: m/z=666 [M+H]⁺.

### Example 230. (4aS,5aR)-N-(1-((3R,4R)-1-(((3S)-1-(4-(2,6-dioxopiperidin-3-yl)phenyl)pyrrolidin-3-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (101)

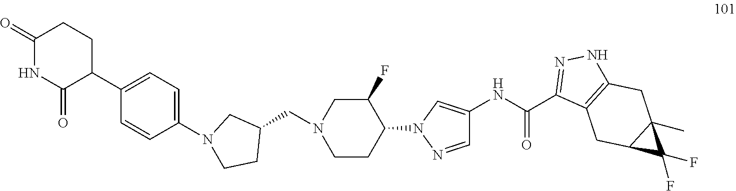 [SMILES: C[C@@]12Cc3[nH]nc(C(=O)Nc4cnn([C@@H]5CCN(C[C@@H]6CCN(c7ccc(C8CCC(=O)NC8=O)cc7)C6)C[C@H]5F)c4)c3C[C@@H]1C2(F)F]

Using general procedure 15, (4aS,5aR)-5,5-difluoro-N-(1-((3R,4R)-3-fluoropiperidin-4-yl)-1H-pyrazol-4-yl)-5a-methyl-1,4,4a,5,5a,6-hexahydrocyclopropa[f]indazole-3-carboxamide (15 mg, 0.038 mmol) was treated with (3R)-1-(4-(2,6-dioxopiperidin-3-yl)phenyl)pyrrolidine-3-carbaldehyde (12 mg, 0.042 mmol) to afford the title compound (18 mg, 0.027 mmol, 70%). ¹H NMR (500 MHz, (CD₃)₂SO) δ 12.96 (s, 1H), 10.74 (s, 1H), 10.16 (s, 1H), 8.09 (s, 1H), 7.70 (s, 1H), 6.99 (d, J=8.2 Hz, 2H), 6.49 (d, J=8.1 Hz, 2H), 4.96-4.73 (m, 1H), 4.28 (dt, J=15.6, 8.3 Hz, 1H), 3.68 (dd, J=10.7, 5.0 Hz, 1H), 3.40-3.34 (m, 1H), 3.25-3.15 (m, 1H), 3.13-2.89 (m, 5H), 2.86-2.78 (m, 1H), 2.61 (ddt, J=19.3, 13.7, 6.6 Hz, 2H), 2.44 (dt, J=17.4, 4.9 Hz, 3H), 2.22-1.89 (m, 4H), 1.78 (dd, J=15.6, 5.7 Hz, 1H), 1.71 (dq, J=14.8, 7.7 Hz, 1H), 1.35 (s, 3H). LCMS: C₃₄H₃₉F₃N₈O₃ requires: 665, found: m/z=666 [M+H]⁺.

### Example 231. (4aS,5aR)-N-(1-((3S,4S)-1-(((3R)-1-(2-(2,6-dioxopiperidin-3-yl)-1,3-dioxoisoindolin-5-yl)pyrrolidin-3-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (102)

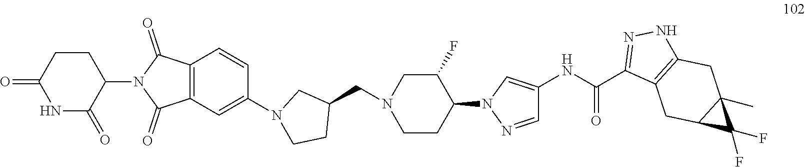 [SMILES: C[C@@]12Cc3[nH]nc(C(=O)Nc4cnn([C@H]5CCN(C[C@H]6CCN(c7ccc8c(c7)C(=O)N(C7CCC(=O)NC7=O)C8=O)C6)C[C@@H]5F)c4)c3C[C@@H]1C2(F)F]

Using general procedure 15, (4aS,5aR)-5,5-difluoro-N-(1-((3S,4S)-3-fluoropiperidin-4-yl)-1H-pyrazol-4-yl)-5a-methyl-1,4,4a, 5, 5a,6-hexahydrocyclopropa[f]indazole-3-carboxamide (15 mg, 0.038 mmol) was treated with (3S)-1-(2-(2,6-dioxopiperidin-3-yl)-1,3-dioxoisoindolin-5-yl)pyrrolidine-3-carbaldehyde (12 mg, 0.042 mmol) to afford the title compound (17 mg, 0.023 mmol, 61%). ¹H NMR (500 MHz, (CD3)₂SO) δ 12.96 (s, 1H), 11.06 (s, 1H), 10.16 (s, 1H), 8.09 (s, 1H), 7.70 (s, 1H), 7.64 (d, J=8.3 Hz, 1H), 6.93 (d, J=2.3 Hz, 1H), 6.83 (dd, J=8.6, 2.2 Hz, 1H), 5.05 (dd, J=12.9, 5.4 Hz, 1H), 4.96-4.77 (m, 1H), 4.29 (dt, J=16.3, 11.3 Hz, 1H), 3.61 (dd, J=10.3, 7.2 Hz, 1H), 3.51 (td, J=9.3, 8.2, 4.1 Hz, 1H), 3.45-3.36 (m, 1H), 3.15 (dd, J=10.3, 6.9 Hz, 1H), 3.10-2.99 (m, 2H), 2.96-2.79 (m, 2H), 2.69-2.53 (m, 2H), 2.22-1.92 (m, 5H), 1.82-1.71 (m, 2H), 1.35 (s, 2H). LCMS: C₃₆H₃₈F₃N₉O₅ requires: 734, found: m/z=735 [M+H]⁺.

### Example 232. (4aS,5aR)-N-(1-((3R,4R)-1-(((3R)-1-(2-(2,6-dioxopiperidin-3-yl)-1,3-dioxoisoindolin-5-yl)pyrrolidin-3-yl)methyl)-3-fluoropiperidin-4-yl)-1H-pyrazol-4-yl)-5,5-difluoro-5a-methyl-1,4,4a,5,5a,6-hexahydrocyclopropa[f]indazole-3-carboxamide (103)

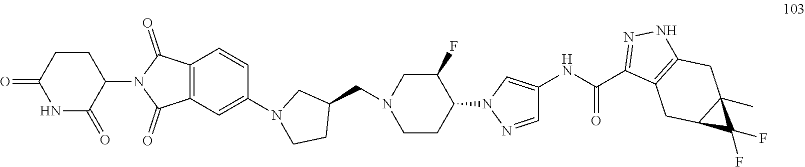 [SMILES: C[C@@]12Cc3[nH]nc(C(=O)Nc4cnn([C@@H]5CCN(C[C@H]6CCN(c7ccc8c(c7)C(=O)N(C7CCC(=O)NC7=O)C8=O)C6)C[C@H]5F)c4)c3C[C@@H]1C2(F)F]

Using general procedure 15, (4aS,5aR)-5,5-difluoro-N-(1-((3R,4R)-3-fluoropiperidin-4-yl)-1H-pyrazol-4-yl)-5a-methyl-1,4,4a,5,5a,6-hexahydrocyclopropa[f]indazole-3-carboxamide (14 mg, 0.036 mmol) was treated with (3S)-1-(2-(2,6-dioxopiperidin-3-yl)-1,3-dioxoisoindolin-5-yl)pyrrolidine-3-carbaldehyde (14 mg, 0.039 mmol) to afford the title compound (17 mg, 0.021 mmol, 60%). ¹H NMR (500 MHz, (CD₃)₂SO) δ 12.98 (s, 1H), 11.08 (s, 1H), 10.17 (s, 1H), 8.10 (s, 1H), 7.71 (s, 1H), 7.65 (d, J=8.4 Hz, 1H), 6.93 (d, J=2.3 Hz, 1H), 6.84 (dd, J=8.6, 2.2 Hz, 1H), 5.07 (dd, J=12.9, 5.4 Hz, 1H), 4.87 (dtd, J=50.1, 9.6, 4.9 Hz, 1H), 4.36-4.25 (m, 1H), 3.64-3.49 (m, 2H), 3.46-3.37 (m, 1H), 3.21-3.11 (m, 1H), 3.08 (d, J=10.4 Hz, 2H), 3.05-2.97 (m, 1H), 2.87 (tdd, J=20.3, 14.4, 5.7 Hz, 2H), 2.70-2.54 (m, 2H), 2.25-1.94 (m, 3H), 1.78 (dq, J=15.8, 8.6, 7.9 Hz, 2H), 1.37 (s, 2H), 1.25 (s, 1H). LCMS: C₃₆H₃₈F₃N₉O₅ requires: 734, found: m/z=735 [M+H]⁺.

### Example 233. (6S)—N-(1-((3S,4S)-1-((1-(4-(2,6-dioxopiperidin-3-yl)phenyl)piperidin-4-yl)methyl)-3-fluoropiperidin-4-yl)-1H-pyrazol-4-yl)-6-(methoxymethyl)-6-methyl-4,5,6,7-tetrahydro-1H-indazole-3-carboxamide (104) (*Arbitrarily Assigned)

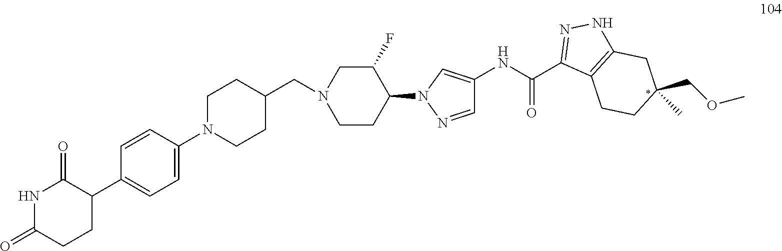 [SMILES: COC[C@@]1(C)CCc2c(C(=O)Nc3cnn([C@H]4CCN(CC5CCN(c6ccc(C7CCC(=O)NC7=O)cc6)CC5)C[C@@H]4F)c3)n[nH]c2C1]

Using general procedure 15, (S)—N-(1-((3S,4S)-3-fluoropiperidin-4-yl)-1H-pyrazol-4-yl)-6-(methoxymethyl)-6-methyl-4,5,6,7-tetrahydro-1H-indazole-3-carboxamide (BBX₂₃) (17 mg, 0.043 mmol) was treated with 1-(4-(2,6-dioxopiperidin-3-yl)phenyl)piperidine-4-carbaldehyde (HCB62) (13 mg, 0.043 mmol) to afford the title compound (16 mg, 0.017 mmol, 55%). ¹H NMR (500 MHz, (CD₃)₂SO) δ 12.82 (s, 1H), 10.76 (s, 1H), 10.09 (s, 1H), 8.08 (s, 1H), 7.70 (s, 1H), 7.03 (d, J=8.1 Hz, 2H), 6.89 (d, J=8.1 Hz, 2H), 4.91-4.72 (m, 1H), 4.27 (d, J=9.1 Hz, 1H), 3.71 (dd, J=10.9, 4.9 Hz, 1H), 3.66 (d, J=12.0 Hz, 2H), 3.27 (s, 4H), 3.15 (s, 2H), 2.91-2.85 (m, 1H), 2.74-2.57 (m, 5H), 2.54 (d, J=16.1 Hz, 2H), 2.45 (dd, J=17.3, 4.6 Hz, 1H), 2.38-2.23 (m, 3H), 2.11 (ddd, J=20.9, 14.4, 10.9 Hz, 3H), 2.05-1.93 (m, 1H), 1.80 (s, 2H), 1.59 (p, J=6.5 Hz, 1H), 1.48 (dt, J=12.7, 6.0 Hz, 1H), 1.22 (d, J=19.4 Hz, 3H), 0.93 (s, 3H). LCMS: C₃₆H₄₇FN₈O₄ requires: 675, found: m/z=676 [M+H]⁺.

### Example 234. (6R)—N-(1-((3S,4S)-1-((1-(4-(2,6-dioxopiperidin-3-yl)phenyl)piperidin-4-yl)methyl)-3-fluoropiperidin-4-yl)-1H-pyrazol-4-yl)-6-(methoxymethyl)-6-methyl-4,5,6,7-tetrahydro-1H-indazole-3-carboxamide (105) (*Arbitrarily Assigned)

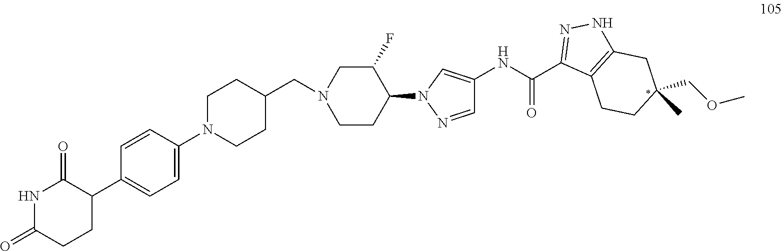 [SMILES: COC[C@]1(C)CCc2c(C(=O)Nc3cnn([C@H]4CCN(CC5CCN(c6ccc(C7CCC(=O)NC7=O)cc6)CC5)C[C@@H]4F)c3)n[nH]c2C1]

Using general procedure 15, (R)—N-(1-((3S,4S)-3-fluoropiperidin-4-yl)-1H-pyrazol-4-yl)-6-(methoxymethyl)-6-methyl-4,5,6,7-tetrahydro-1H-indazole-3-carboxamide (BBX₂₄) (15 mg, 0.038 mmol) was treated with 1-(4-(2,6-dioxopiperidin-3-yl)phenyl)piperidine-4-carbaldehyde (HCB62) (12 mg, 0.038 mmol) to afford the title compound (18 mg, 0.027 mmol, 70%). 1H NMR (500 MHz, (CD₃)₂SO) δ 12.82 (s, 1H), 10.76 (s, 1H), 10.09 (s, 1H), 8.08 (s, 1H), 7.70 (s, 1H), 7.03 (d, J=8.2 Hz, 2H), 6.89 (d, J=8.3 Hz, 2H), 4.91-4.73 (m, 1H), 4.31-4.25 (m, 1H), 3.71 (dd, J=10.9, 4.9 Hz, 1H), 3.66 (d, J=12.0 Hz, 2H), 3.30-3.29 (m, OH), 3.27 (s, 3H), 3.15 (s, 2H), 2.88 (d, J=10.1 Hz, 1H), 2.75-2.54 (m, 5H), 2.46 (d, J=17.3 Hz, 1H), 2.38-2.24 (m, 3H), 2.17-2.05 (m, 3H), 2.05-1.95 (m, 1H), 1.80 (s, 2H), 1.68 (s, 1H), 1.59 (dt, J=13.9, 7.0 Hz, 1H), 1.52-1.45 (m, 1H), 1.27-1.14 (m, 3H), 0.93 (s, 3H). LCMS: C₃₆H₄₇FN₈O₄ requires: 675, found: m/z=676 [M+H]⁺.

### Example 235. (6S)—N-(1-((3R,4R)-1-((1-(4-(2,6-dioxopiperidin-3-yl)phenyl)piperidin-4-yl)methyl)-3-fluoropiperidin-4-yl)-1H-pyrazol-4-yl)-6-(methoxymethyl)-6-methyl-4,5,6,7-tetrahydro-1H-indazole-3-carboxamide (106) (*Arbitrarily Assigned)

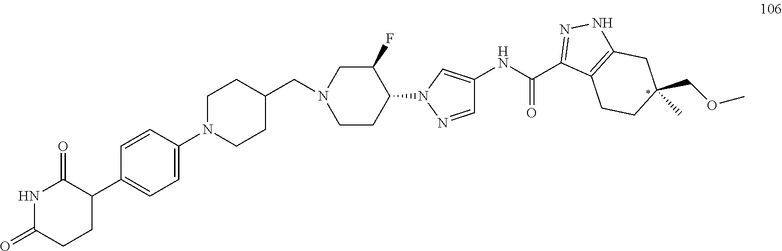 [SMILES: COC[C@@]1(C)CCc2c(C(=O)Nc3cnn([C@@H]4CCN(CC5CCN(c6ccc(C7CCC(=O)NC7=O)cc6)CC5)C[C@H]4F)c3)n[nH]c2C1]

Using general procedure 15, (S)—N-(1-((3R,4R)-3-fluoropiperidin-4-yl)-1H-pyrazol-4-yl)-6-(methoxymethyl)-6-methyl-4,5,6,7-tetrahydro-1H-indazole-3-carboxamide (BBX₂₅) (15 mg, 0.038 mmol) was treated with 1-(4-(2,6-dioxopiperidin-3-yl)phenyl)piperidine-4-carbaldehyde (HCB62) (12 mg, 0.038 mmol) to afford the title compound (14 mg, 0.021 mmol, 53%). ¹H NMR (500 MHz, (CD₃)₂SO) δ 12.82 (s, 1H), 10.76 (s, 1H), 10.09 (s, 1H), 8.08 (s, 1H), 7.70 (s, 1H), 7.03 (d, J=8.1 Hz, 2H), 6.89 (d, J=8.2 Hz, 2H), 4.92-4.71 (m, 1H), 4.28 (s, 1H), 3.72 (dd, J=10.8, 5.0 Hz, 1H), 3.66 (d, J=12.0 Hz, 2H), 3.27 (s, 3H), 3.15 (s, 2H), 2.88 (d, J=10.0 Hz, 1H), 2.62 (ddd, J=31.7, 24.1, 18.7 Hz, 5H), 2.44 (d, J=4.7 Hz, OH), 2.33 (d, J=16.2 Hz, 1H), 2.29 (s, 2H), 2.18-1.91 (m, 4H), 1.80 (s, 1H), 1.68 (s, 1H), 1.58 (q, J=6.9 Hz, 1H), 1.52-1.45 (m, 1H), 1.25-1.18 (m, 2H), 0.93 (s, 3H). LCMS: C₃₆H₄₇FN₈O₄ requires: 675, found: m/z=676 [M+H]⁺.

### Example 236. (6R)—N-(1-((3R,4R)-1-((1-(4-(2,6-dioxopiperidin-3-yl)phenyl)piperidin-4-yl)methyl)-3-fluoropiperidin-4-yl)-1H-pyrazol-4-yl)-6-(methoxymethyl)-6-methyl-4,5,6,7-tetrahydro-1H-indazole-3-carboxamide (107) (*Arbitrarily Assigned)

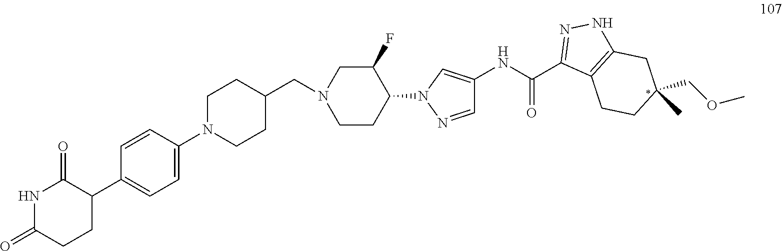 [SMILES: COC[C@]1(C)CCc2c(C(=O)Nc3cnn([C@@H]4CCN(CC5CCN(c6ccc(C7CCC(=O)NC7=O)cc6)CC5)C[C@H]4F)c3)n[nH]c2C1]

Using general procedure 15, (R)—N-(1-((3R,4R)-3-fluoropiperidin-4-yl)-1H-pyrazol-4-yl)-6-(methoxymethyl)-6-methyl-4,5,6,7-tetrahydro-1H-indazole-3-carboxamide (BBX₂₆) (15 mg, 0.038 mmol) was treated with 1-(4-(2,6-dioxopiperidin-3-yl)phenyl)piperidine-4-carbaldehyde (HCB62) (12 mg, 0.038 mmol) to afford the title compound (13.3 mg, 0.019 mmol, 50%). ¹H NMR (500 MHz, (CD₃)₂SO) δ 12.82 (s, 1H), 10.76 (s, 1H), 10.09 (s, 1H), 8.08 (s, 1H), 7.70 (s, 1H), 7.03 (d, J=8.1 Hz, 2H), 6.89 (d, J=8.2 Hz, 2H), 4.91-4.72 (m, 1H), 4.31-4.25 (m, 1H), 3.72 (dd, J=10.9, 4.9 Hz, 1H), 3.66 (d, J=12.0 Hz, 2H), 3.27 (s, 3H), 3.15 (s, 2H), 2.88 (d, J=10.1 Hz, 1H), 2.74-2.57 (m, 5H), 2.54 (d, J=15.7 Hz, 1H), 2.49-2.41 (m, 1H), 2.38-2.24 (m, 3H), 2.19-1.89 (m, 4H), 1.80 (s, 2H), 1.68 (s, 1H), 1.59 (dt, J=13.9, 6.8 Hz, 1H), 1.52-1.45 (m, 1H), 1.25-1.18 (m, 2H), 0.93 (s, 3H). LCMS: C₃₆H₄₇FN₈O₄ requires: 675, found: m/z=676 [M+H]⁺.

### Example 237. (4aS,5aR)-N-(4-(1-((1-(4-(2,6-dioxopiperidin-3-yl)phenyl)piperidin-4-yl)methyl)piperidin-4-yl)phenyl)-5,5-difluoro-5a-methyl-1,4,4a,5,5a,6-hexahydrocyclopropa[f]indazole-3-carboxamide (108)

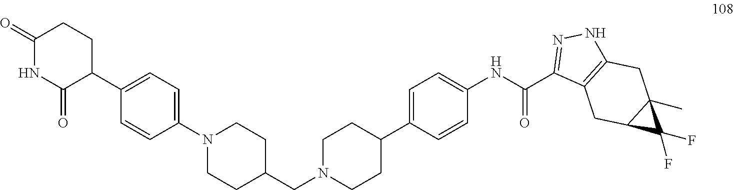 [SMILES: C[C@@]12Cc3[nH]nc(C(=O)Nc4ccc(C5CCN(CC6CCN(c7ccc(C8CCC(=O)NC8=O)cc7)CC6)CC5)cc4)c3C[C@@H]1C2(F)F]

Using general procedure 15, (4aS,5aR)-5,5-difluoro-5a-methyl-N-(4-(piperidin-4-yl)phenyl)-1,4,4a,5,5a,6-hexahydrocyclopropa[f]indazole-3-carboxamide (BBX₂₇) (17 mg, 0.044 mmol) was treated with 1-(4-(2,6-dioxopiperidin-3-yl)phenyl)piperidine-4-carbaldehyde (HCB62) (15 mg, 0.048 mmol) to afford the title compound (4.7 mg, 0.0066 mmol, 15%). LCMS: C₃₈H₄₄F₂N₆O₃ requires: 670, found: m/z=671 [M+H]⁺.

### Example 238. (4aS,5aR)-N-(4-(1-((1-(5-(2,6-dioxopiperidin-3-yl)pyridin-2-yl)piperidin-4-yl)methyl)piperidin-4-yl)phenyl)-5,5-difluoro-5a-methyl-1,4,4a,5,5a,6-hexahydrocyclopropa[f]indazole-3-carboxamide (109)

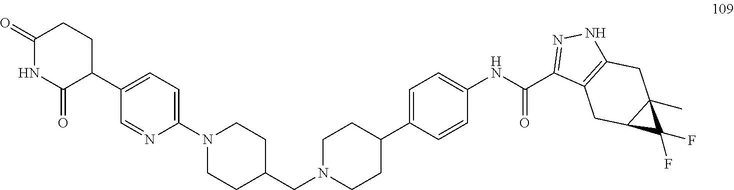 [SMILES: C[C@@]12Cc3[nH]nc(C(=O)Nc4ccc(C5CCN(CC6CCN(c7ccc(C8CCC(=O)NC8=O)cn7)CC6)CC5)cc4)c3C[C@@H]1C2(F)F]

Using general procedure 15, (4aS,5aR)-5,5-difluoro-5a-methyl-N-(4-(piperidin-4-yl)phenyl)-1,4,4a,5,5a,6-hexahydrocyclopropa[f]indazole-3-carboxamide (BBX₂₇) (27 mg, 0.069 mmol) was treated with 1-(5-(2,6-dioxopiperidin-3-yl)pyridin-2-yl)piperidine-4-carbaldehyde (HCB60) (23 mg, 0.075 mmol) to afford the title compound (3.8 mg, 0.055 mmol, 8%). LCMS: C₃₇H₄₃F₂N₇O₃ requires: 671, found: m/z=672 [M+H]⁺.

### Example 239. (4aS,5aR)-N-(6-(1-((1-(4-(2,6-dioxopiperidin-3-yl)phenyl)piperidin-4-yl)methyl)piperidin-4-yl)pyridin-3-yl)-5,5-difluoro-5a-methyl-1,4,4a,5,5a,6-hexahydrocyclopropa[f]indazole-3-carboxamide (110)

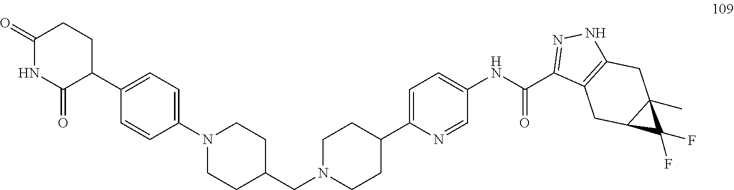 [SMILES: C[C@@]12Cc3[nH]nc(C(=O)Nc4ccc(C5CCN(CC6CCN(c7ccc(C8CCC(=O)NC8=O)cc7)CC6)CC5)nc4)c3C[C@@H]1C2(F)F]

Using general procedure 15, (4aS,5aR)-5,5-difluoro-5a-methyl-N-(6-(piperidin-4-yl)pyridin-3-yl)-1,4,4a,5,5a,6-hexahydrocyclopropa[f]indazole-3-carboxamide (BBX₂₈) (27 mg, 0.070 mmol) was treated with 1-(4-(2,6-dioxopiperidin-3-yl)phenyl)piperidine-4-carbaldehyde (HCB62) (23 mg, 0.077 mmol) to afford the title compound (2.2 mg, 0.0032 mmol, 4.6%). LCMS: C₃₇H₄₃F₂N₇O₃ requires: 671, found: m/z=672 [M+H]⁺ Example 240. (4aS,5aR)-N-(6-(1-((1-(5-(2,6-dioxopiperidin-3-yl)pyridin-2-yl)piperidin-4-yl)methyl)piperidin-4-yl)pyridin-3-yl)-5,5-difluoro-5a-methyl-1,4,4a,5,5a,6-hexahydrocyclopropa[f]indazole-3-carboxamide (111)

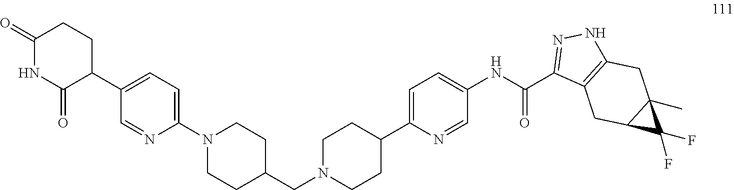 [SMILES: C[C@@]12Cc3[nH]nc(C(=O)Nc4ccc(C5CCN(CC6CCN(c7ccc(C8CCC(=O)NC8=O)cn7)CC6)CC5)nc4)c3C[C@@H]1C2(F)F]

Using general procedure 15, (4aS,5aR)-5,5-difluoro-5a-methyl-N-(6-(piperidin-4-yl)pyridin-3-yl)-1,4,4a,5,5a,6-hexahydrocyclopropa[f]indazole-3-carboxamide (BBX₂₈) (29 mg, 0.074 mmol) was treated with 1-(5-(2,6-dioxopiperidin-3-yl)pyridin-2-yl)piperidine-4-carbaldehyde (HCB60) (24 mg, 0.081 mmol) to afford the title compound (2.1 mg, 0.0030 mmol, 4.0%). LCMS: C₃₆H₄₂F₂N₈O₃ requires: 672, found: m/z=673 [M+H]⁺.

### Example 241. (4aS,5aR)-N-(1-{1-[(1-{4-[(3RS)-2,6-dioxopiperidin-3-yl]-2-fluorophenyl}piperidin-4-yl)methyl]piperidin-4-yl}-1H-pyrazol-4-yl)-5,5-difluoro-5a-methyl-1H,4H,4aH,5H,5aH,6H-cyclopropa[f]indazole-3-carboxamide (112)

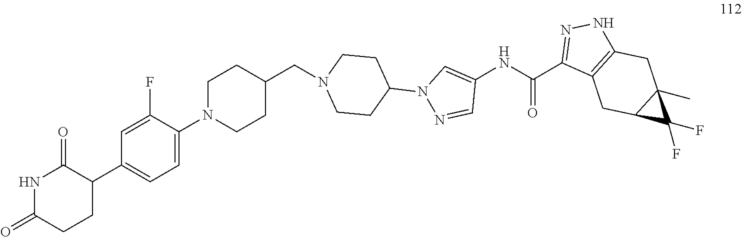 [SMILES: C[C@@]12Cc3[nH]nc(C(=O)Nc4cnn(C5CCN(CC6CCN(c7ccc(C8CCC(=O)NC8=O)cc7F)CC6)CC5)c4)c3C[C@@H]1C2(F)F]

This compound was synthesized following the general procedure 15. LCMS: C₃₅H₄₁F₃N₈O₃ requires: 678.3, found: m/z=679.7 [M+H]⁺.

### Example 242. (4aS,5aR)-N-{1-[1-({1-[5-(2,4-dioxo-1,3-diazinan-1-yl)pyridin-2-yl]piperidin-4-yl}methyl)piperidin-4-yl]-1H-pyrazol-4-yl}-5,5-difluoro-5a-methyl-1H,4H,4aH,5H,5aH,6H-cyclopropa[f]indazole-3-carboxamide (113)

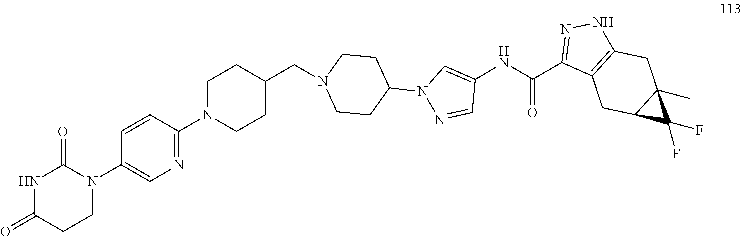 [SMILES: C[C@@]12Cc3[nH]nc(C(=O)Nc4cnn(C5CCN(CC6CCN(c7ccc(N8CCC(=O)NC8=O)cn7)CC6)CC5)c4)c3C[C@@H]1C2(F)F]

This compound was synthesized following the general procedure 15. LCMS: C₃₃H₄₀F₂N₁₀O₃ requires: 662.3, found: m/z=663.6 [M+H]⁺.

### Example 243. (4aS,5aR)-N-(1-{1-[(1-{5-[(3RS)-2,6-dioxopiperidin-3-yl]pyridin-2-yl}piperidin-4-yl)methyl]piperidin-4-yl}-1H-pyrazol-4-yl)-5,5-difluoro-5a-methyl-1H,4H,4aH,5H,5aH,6H-cyclopropa[f]indazole-3-carboxamide (114)

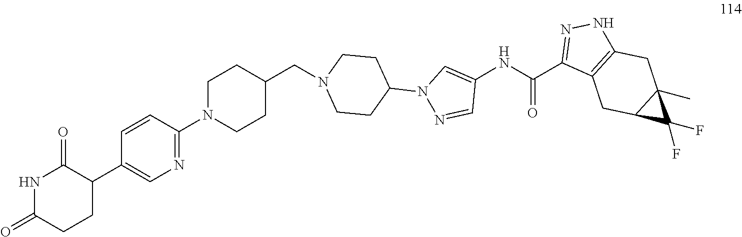 [SMILES: C[C@@]12Cc3[nH]nc(C(=O)Nc4cnn(C5CCN(CC6CCN(c7ccc(C8CCC(=O)NC8=O)cn7)CC6)CC5)c4)c3C[C@@H]1C2(F)F]

This compound was synthesized following the general procedure 15. LCMS: C₃₄H₄₁F₂N₉O₃ requires: 661.3, found: m/z=662.4 [M+H]⁺.

### Example 244. (4aS,5aR)-N-(1-{1-[(1-{5-[(3RS)-2,6-dioxopiperidin-3-yl]pyrimidin-2-yl}piperidin-4-yl)methyl]piperidin-4-yl}-1H-pyrazol-4-yl)-5,5-difluoro-5a-methyl-1H,4H,4aH,5H,5aH,6H-cyclopropa[f]indazole-3-carboxamide (115)

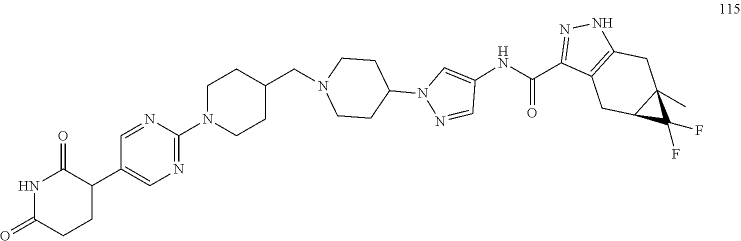 [SMILES: C[C@@]12Cc3[nH]nc(C(=O)Nc4cnn(C5CCN(CC6CCN(c7ncc(C8CCC(=O)NC8=O)cn7)CC6)CC5)c4)c3C[C@@H]1C2(F)F]

This compound was synthesized following the general procedure 15. LCMS: C₃₃H₄₀F₂N₁₀O₃ requires: 662.3, found: m/z=663.4 [M+H]⁺. ¹H NMR (500 MHz, DMSO-d₆) δ 12.99 (s, 1H), 10.88 (s, 1H), 10.20 (s, 1H), 8.94 (s, 1H), 8.25 (s, 2H), 8.09 (s, 1H), 7.69 (s, 1H), 4.68 (d, J=13.0 Hz, 2H), 4.46 (s, 1H), 3.75 (dd, J=12.9, 4.8 Hz, 2H), 3.14 (t, J=10.6 Hz, 4H), 3.09-3.02 (m, 5H), 2.94 (t, J=12.7 Hz, 3H), 2.84 (d, J=17.4 Hz, 1H), 2.77-2.61 (m, 2H), 2.24 (t, J=6.6 Hz, 6H), 2.09 (s, 1H), 1.99 (d, J=12.5 Hz, 2H), 1.82 (t, J=17.8 Hz, 4H), 1.37 (s, 4H), 1.23 (d, J=15.5 Hz, 1H), 1.20-1.07 (m, 2H).

### Example 245. (4aS,5aR)-N-(1-{1-[(1-{4-[(3S)-2,6-dioxopiperidin-3-yl]phenyl}piperidin-4-yl)methyl]piperidin-4-yl}-1H-pyrazol-4-yl)-5,5-difluoro-5a-methyl-1H,4H,4aH,5H,5aH,6H-cyclopropa[f]indazole-3-carboxamide (116)

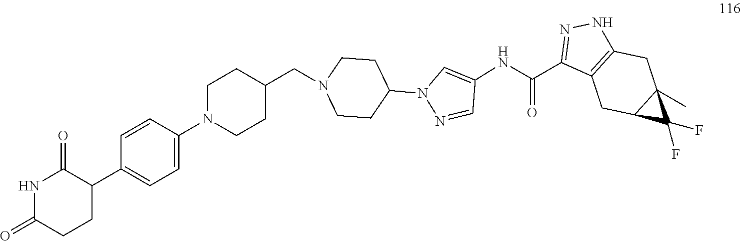 [SMILES: C[C@@]12Cc3[nH]nc(C(=O)Nc4cnn(C5CCN(CC6CCN(c7ccc(C8CCC(=O)NC8=O)cc7)CC6)CC5)c4)c3C[C@@H]1C2(F)F]

This compound was synthesized following the general procedure 15. ¹H NMR (500 MHz, DMSO-d₆) δ 12.96 (s, 1H), 11.92 (s, 1H), 10.77 (s, 1H), 10.11 (s, 1H), 8.01 (s, 1H), 7.64 (s, 1H), 7.04 (d, J=8.4 Hz, 2H), 6.90 (d, J=8.4 Hz, 2H), 5.77 (s, 2H), 4.10 (q, J=7.7, 6.5 Hz, 1H), 3.73 (dd, J=11.0, 4.9 Hz, 1H), 3.67 (d, J=12.1 Hz, 2H), 3.18 (d, J=5.1 Hz, 1H), 3.10-2.98 (m, 3H), 2.94 (d, J=11.1 Hz, 2H), 2.83 (d, J=17.1 Hz, 1H), 2.65 (dd, J=13.6, 9.8 Hz, 4H), 2.21 (d, J=7.1 Hz, 2H), 2.18-1.94 (m, 7H), 1.92 (s, 4H), 1.79 (d, J=9.0 Hz, 4H), 1.67 (s, 1H), 1.36 (s, 3H), 1.30-1.16 (m, 3H).

### Example 246. (4aS,5aR)-N-(1-{1-[(1-{4-[(3R)-2,6-dioxopiperidin-3-yl]phenyl}piperidin-4-yl)methyl]piperidin-4-yl}-1H-pyrazol-4-yl)-5,5-difluoro-5a-methyl-1H,4H,4aH,5H,5aH,6H-cyclopropa[f]indazole-3-carboxamide (117)

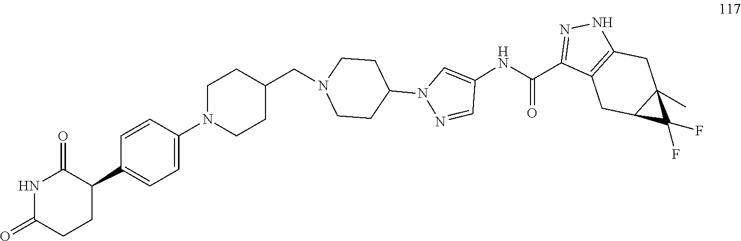 [SMILES: C[C@@]12Cc3[nH]nc(C(=O)Nc4cnn(C5CCN(CC6CCN(c7ccc([C@H]8CCC(=O)NC8=O)cc7)CC6)CC5)c4)c3C[C@@H]1C2(F)F]

This compound was synthesized following the general procedure 15. LCMS: C₃₅H₄₂F₂N₈O₃ requires: 660.3, found: m/z=661.7 [M+H]⁺. ¹H NMR (500 MHz, DMSO-d₆) δ 12.97 (s, 1H), 10.78 (s, 1H), 10.12 (s, 1H), 8.01 (s, 1H), 7.64 (s, 1H), 7.04 (d, J=8.0 Hz, 2H), 6.90 (d, J=8.4 Hz, 2H), 4.10 (dd, J=10.4, 5.2 Hz, 1H), 3.84-3.48 (m, 5H), 3.21-3.12 (m, 2H), 3.12-2.98 (m, 4H), 2.94 (d, J=10.9 Hz, 2H), 2.83 (d, J=17.0 Hz, 1H), 2.65 (td, J=11.7, 4.7 Hz, 3H), 2.21 (d, J=7.2 Hz, 2H), 2.13 (t, J=10.5 Hz, 1H), 2.10-1.84 (m, 7H), 1.84-1.73 (m, 3H), 1.67 (s, 1H), 1.37 (s, 4H), 1.30-1.21 (m, 9H).

### Example 247. (4aS,5aR)-N-(1-{1-[(1-{4-[(3R)-2,6-dioxopiperidin-3-yl]-2-fluorophenyl}piperidin-4-yl)methyl]piperidin-4-yl}-1H-pyrazol-4-yl)-5,5-difluoro-5a-methyl-1H,4H,4aH,5H,5aH,6H-cyclopropa[f]indazole-3-carboxamide (118)

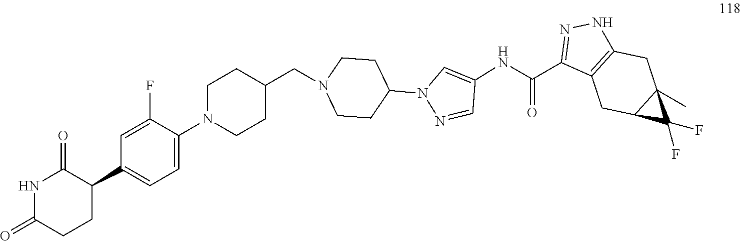 [SMILES: C[C@@]12Cc3[nH]nc(C(=O)Nc4cnn(C5CCN(CC6CCN(c7ccc([C@H]8CCC(=O)NC8=O)cc7F)CC6)CC5)c4)c3C[C@@H]1C2(F)F]

This compound was synthesized following the general procedure 15. LCMS: C₃₅H₄₁F₃N₈O₃ requires: 678.3, found: m/z=679.4 [M+H]⁺.

### Example 248. (4aS,5aR)-N-[1-({1-[(1-{4-[(3RS)-2,6-dioxopiperidin-3-yl]phenyl}piperidin-4-yl)methyl]piperidin-4-yl}methyl)-1H-pyrazol-4-yl]-5,5-difluoro-5a-methyl-1H,4H,4aH,5H,5aH,6H-cyclopropa[f]indazole-3-carboxamide (119)

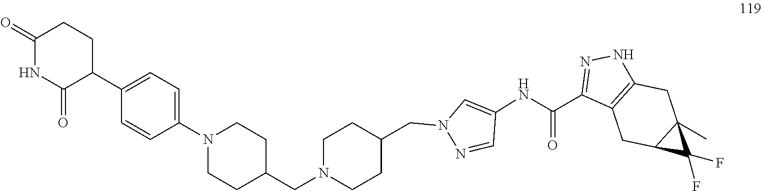 [SMILES: C[C@@]12Cc3[nH]nc(C(=O)Nc4cnn(CC5CCN(CC6CCN(c7ccc(C8CCC(=O)NC8=O)cc7)CC6)CC5)c4)c3C[C@@H]1C2(F)F]

This compound was synthesized following the general procedure 15. LCMS: C₃₆H₄₄F₂N₈O₃ requires: 674.4, found: m/z=675.7 [M+H]⁺. ¹H NMR (500 MHz, DMSO-d₆) δ 12.98 (s, 1H), 10.78 (s, 1H), 10.15 (s, 1H), 8.01 (s, 1H), 7.62 (s, 1H), 7.04 (d, J=8.1 Hz, 2H), 6.89 (d, J=8.2 Hz, 2H), 5.77 (d, J=2.4 Hz, 3H), 4.22-3.86 (m, 3H), 3.69 (dd, J=33.8, 10.6 Hz, 4H), 3.25-3.14 (m, 3H), 3.14-2.97 (m, 4H), 2.84 (d, J=17.0 Hz, 3H), 2.63 (t, J=12.0 Hz, 3H), 2.24-2.00 (m, 4H), 1.79 (t, J=13.0 Hz, 6H), 1.48 (s, 2H), 1.37 (s, 4H), 1.25 (s, 5H), 1.11 (t, J=7.3 Hz, 2H).

### Example 249. (4aS,5aR)-N-[1-({1-[(1-{5-[(3RS)-2,6-dioxopiperidin-3-yl]pyridin-2-yl}piperidin-4-yl)methyl]piperidin-4-yl}methyl)-1H-pyrazol-4-yl]-5,5-difluoro-5a-methyl-1H,4H,4aH,5H,5aH,6H-cyclopropa[f]indazole-3-carboxamide (120)

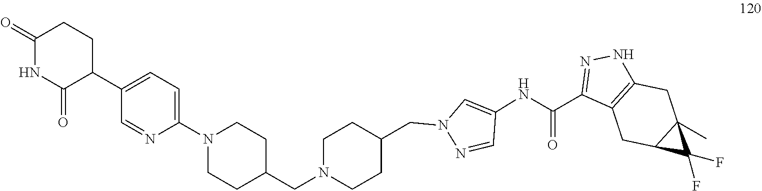 [SMILES: C[C@@]12Cc3[nH]nc(C(=O)Nc4cnn(CC5CCN(CC6CCN(c7ccc(C8CCC(=O)NC8=O)cn7)CC6)CC5)c4)c3C[C@@H]1C2(F)F]

This compound was synthesized following the general procedure 15. LCMS: C₃₅H₄₃F₂N₉O₃ requires: 675.3, found: m/z=676.7 [M+H]⁺. ¹H NMR (500 MHz, DMSO-d₆) δ 12.97 (s, 1H), 11.96 (s, 1H), 10.81 (s, 1H), 10.15 (s, 1H), 7.97 (d, J=30.8 Hz, 2H), 7.62 (s, 1H), 7.36 (d, J=8.7 Hz, 1H), 6.78 (d, J=8.9 Hz, 1H), 4.25 (d, J=12.7 Hz, 2H), 4.10 (d, J=5.8 Hz, 1H), 3.96 (d, J=6.9 Hz, 2H), 3.72 (dd, J=11. 1, 4.3 Hz, 1H), 3.18 (d, J=4.2 Hz, 3H), 3.13-3.01 (m, 3H), 2.76 (ddd, J=52.3, 36.8, 14.9 Hz, 7H), 2.26-2.07 (m, 3H), 2.07-1.88 (m, 6H), 1.77 (dd, J=27.5, 13.4 Hz, 7H), 1.48 (s, 2H), 1.37 (s, 3H), 1.23 (s, 4H), 1.16-1.04 (m, 3H).

### Example 250. N-(1-{1-[(1-{4-[(3S)-2,6-dioxopiperidin-3-yl]phenyl}piperidin-4-yl)methyl]piperidin-4-yl}-1H-pyrazol-4-yl)-6,6-dimethyl-4,5,6,7-tetrahydro-1H-indazole-3-carboxamide (121)

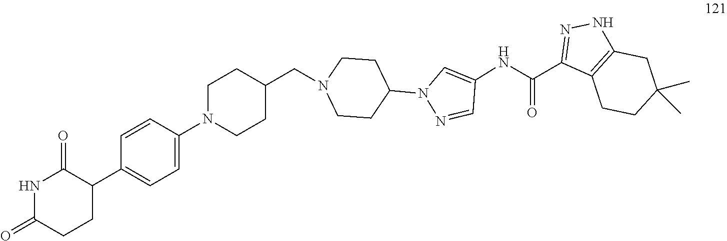 [SMILES: CC1(C)CCc2c(C(=O)Nc3cnn(C4CCN(CC5CCN(c6ccc(C7CCC(=O)NC7=O)cc6)CC5)CC4)c3)n[nH]c2C1]

This compound was synthesized following the general procedure 15. LCMS: C₃₅H₄₆N₈O₃ requires: 626.4, found: m/z=627.6 [M+H]⁺.

### Example 251. (4aS,5aR)-5,5-difluoro-5a-methyl-N-{1-[(1rs,4rs)-4-{[6-(2,4-dioxo-1,3-diazinan-1-yl)-1,2,3,4-tetrahydroisoquinolin-2-yl]methyl}cyclohexyl]-1H-pyrazol-4-yl}-1H,4H,4aH,5H,5aH,6H-cyclopropa[f]indazole-3-carboxamide (122)

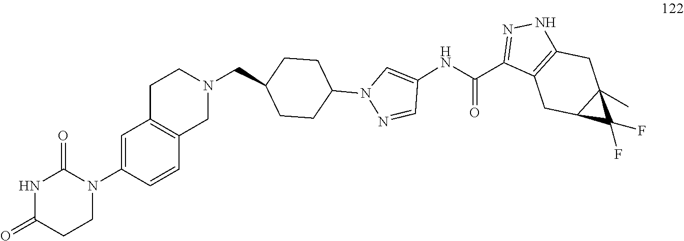

This compound was synthesized following the general procedure 15. LCMS: C₃₃H₃₈F₂N₈O₃ requires: 632.3, found: m/z=633.5 [M+H]⁺.

### Example 252. (4aS,5aR)-5,5-difluoro-5a-methyl-N-{1-[(lsr,4sr)-4-({6-[(3RS)-2,6-dioxopiperidin-3-yl]-1,2,3,4-tetrahydroisoquinolin-2-yl}methyl)cyclohexyl]-1H-pyrazol-4-yl}-1H,4H,4aH,5H,5aH,6H-cyclopropa[f]indazole-3-carboxamide (123)

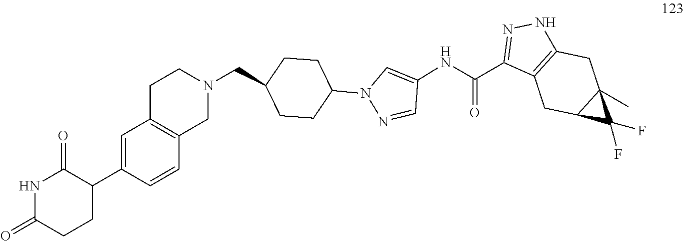

This compound was synthesized following the general procedure 15. LCMS: C₃₄H₃₉F₂N₇O₃ requires: 631.3, found: m/z=632.5 [M+H]⁺.

### Example 253. (4aS,5aR)-5,5-difluoro-5a-methyl-N-{1-[(1rs,4rs)-4-{[4-({5-[(3R)-2,6-dioxopiperidin-3-yl]pyridin-2-yl}oxy)piperidin-1-yl]methyl}cyclohexyl]-1H-pyrazol-4-yl}-1H,4H,4aH,5H,5aH,6H-cyclopropa[f]indazole-3-carboxamide (124)

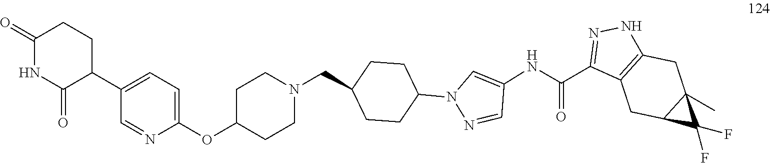

This compound was synthesized following the general procedure 15. LCMS: C₃₅H₄₂F₂N₈O₄ requires: 676.3, found: m/z=677.6 [M+H]⁺.

### Example 254. (4aS,5aR)-5,5-difluoro-5a-methyl-N-{1-[(1rs,4rs)-4-[(4-{5-[(3RS)-2,6-dioxopiperidin-3-yl]pyridin-2-yl}piperazin-1-yl)methyl]cyclohexyl]-1H-pyrazol-4-yl}-1H,4H,4aH,5H,5aH,6H-cyclopropa[f]indazole-3-carboxamide (125)

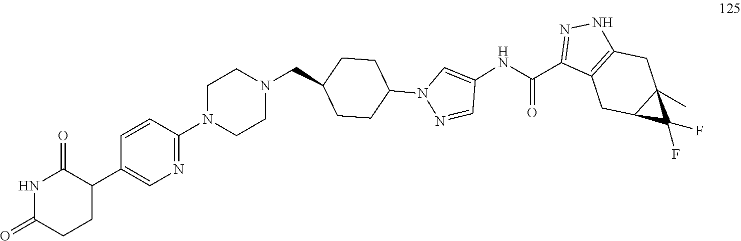

This compound was synthesized following the general procedure 15. LCMS: C₃₄H₄₁F₂N₉O₃ requires: 661.3, found: m/z=662.5 [M+H]⁺.

### Example 255. (4aS,5aR)-5,5-difluoro-5a-methyl-N-{1-[(1rs,4rs)-4-[(4-{4-[(3RS)-2,6-dioxopiperidin-3-yl]phenyl}piperazin-1-yl)methyl]cyclohexyl]-1H-pyrazol-4-yl}-1H,4H,4aH,5H,5aH,6H-cyclopropa[f]indazole-3-carboxamide (126)

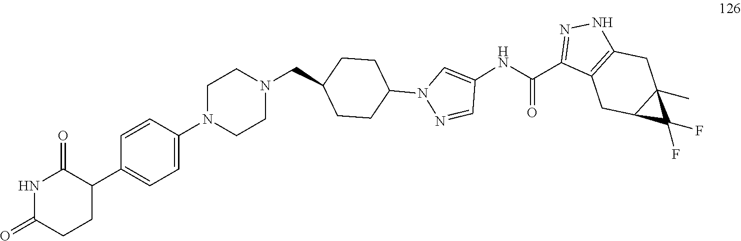

This compound was synthesized following the general procedure 15. LCMS: C₃₅H₄₂F₂N₈O₃ requires: 660.3, found: m/z=661.5 [M+H]⁺.

### Example 256. N-[(3SR)-2,6-dioxopiperidin-3-yl]-5-(4-{[(1rs,4rs)-4-{4-[(4aS,5aR)-5,5-difluoro-5a-methyl-1H,4H,4aH,5H,5aH,6H-cyclopropa[f]indazole-3-amido]-1H-pyrazol-1-yl}cyclohexyl]methyl}piperazin-1-yl)pyridine-2-carboxamide (127)

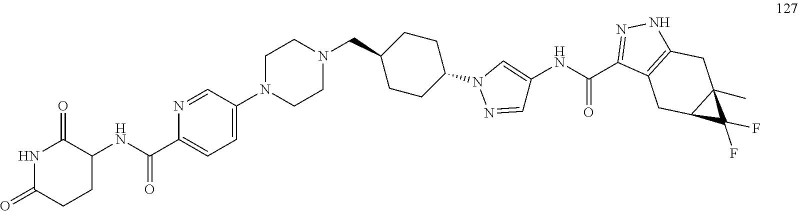 [SMILES: C[C@@]12Cc3[nH]nc(C(=O)Nc4cnn([C@H]5CC[C@H](CN6CCN(c7ccc(C(=O)NC8CCC(=O)NC8=O)nc7)CC6)CC5)c4)c3C[C@@H]1C2(F)F]

This compound was synthesized following the general procedure 15. LCMS: C₃₅H₄₂F₂N₁₀O₄ requires: 704.3, found: m/z=705.5 [M+H]⁺.

### Example 257. (4aS,5aR)-N-[1-(1-{[1-(4-{[(3RS)-2,6-dioxopiperidin-3-yl]carbamoyl}phenyl)piperidin-4-yl]methyl}piperidin-4-yl)-1H-pyrazol-4-yl]-5,5-difluoro-5a-methyl-1H,4H,4aH,5H,5aH,6H-cyclopropa[f]indazole-3-carboxamide (128)

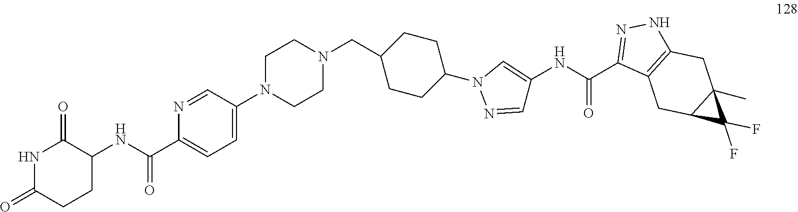 [SMILES: C[C@@]12Cc3[nH]nc(C(=O)Nc4cnn(C5CCC(CN6CCN(c7ccc(C(=O)NC8CCC(=O)NC8=O)nc7)CC6)CC5)c4)c3C[C@@H]1C2(F)F]

This compound was synthesized following the general procedure 15. LCMS: C₃₆H₄₃F₂N₉O₄ requires: 703.3, found: m/z=704.6 [M+H]⁺.

### Example 258. 3-(6-(4-(((S)-4-(2-(6,6-dimethyl-4,5,6,7-tetrahydro-1H-indazol-3-yl)-1H-indole-6-carbonyl)-3-methylpiperazin-1-yl)methyl)piperidin-1-yl)pyridin-3-yl)piperidine-2,6-dione (130)

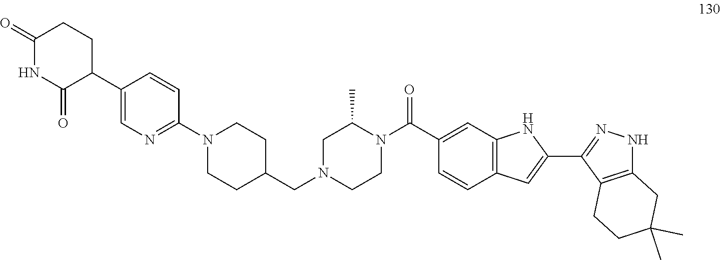 [SMILES: C[C@H]1CN(CC2CCN(c3ccc(C4CCC(=O)NC4=O)cn3)CC2)CCN1C(=O)c1ccc2cc(-c3n[nH]c4c3CCC(C)(C)C4)[nH]c2c1]

Using general procedure 16, (S)-(2-(6,6-dimethyl-4,5,6,7-tetrahydro-1H-indazol-3-yl)-1H-indol-6-yl)(2-methylpiperazin-1-yl)methanone (20 mg, 0.051 mmol) was treated with 1-(5-(2,6-dioxopiperidin-3-yl)pyridin-2-yl)piperidine-4-carbaldehyde (HCB60) (20 mg, 0.066 mmol) to afford the title compound (12 mg, 0.017 mmol, 33%). ¹H NMR (500 MHz, CDCl₃) δ 10.73 (s, 1H), 10.11 (s, 1H), 8.06 (d, J=2.5 Hz, 1H), 7.60 (d, J=8.1 Hz, 1H), 7.46 (s, 1H), 7.34 (d, J=8.9 Hz, 1H), 7.13 (d, J=8.1 Hz, 1H), 6.73-6.66 (m, 2H), 4.33 (s, 1H), 4.28 (d, J=15.0 Hz, 1H), 3.67 (dd, J=11.4, 5.0 Hz, 1H), 3.29 (s, 1H), 2.92-2.55 (m, 5H), 2.45 (s, 2H), 2.31-2.04 (m, 4H), 1.94-1.70 (m, 2H), 1.65 (t, J=6.4 Hz, 2H), 1.61 (s, 1H), 1.42-1.18 (m, 6H), 1.06 (s, 5H). LCMS: C₃₉H₄₈N₈O₃ requires: 676, found: m/z=677 [M+H]⁺.

### Example 259. 3-(6-(4-(((S)-4-(2-(6,6-dimethyl-4,5,6,7-tetrahydro-1H-indazol-3-yl)-1H-indole-6-carbonyl)-2-methylpiperazin-1-yl)methyl)piperidin-1-yl)pyridin-3-yl)piperidine-2,6-dione (131)

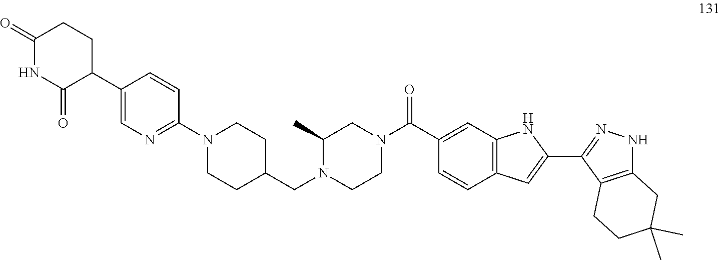 [SMILES: C[C@H]1CN(C(=O)c2ccc3cc(-c4n[nH]c5c4CCC(C)(C)C5)[nH]c3c2)CCN1CC1CCN(c2ccc(C3CCC(=O)NC3=O)cn2)CC1]

Using general procedure 16, (S)-(2-(6,6-dimethyl-4,5,6,7-tetrahydro-1H-indazol-3-yl)-1H-indol-6-yl)(3-methylpiperazin-1-yl)methanone (20 mg, 0.051 mmol) was treated with 1-(5-(2,6-dioxopiperidin-3-yl)pyridin-2-yl)piperidine-4-carbaldehyde (HCB60) (20 mg, 0.066 mmol) to afford the title compound (22 mg, 0.032 mmol, 62%). ¹H NMR (500 MHz, CDCl₃) δ 10.87 (s, 1H), 10.73 (s, 1H), 8.04 (s, 1H), 7.58-7.51 (m, 2H), 7.28 (d, J=8.9 Hz, 1H), 7.08 (d, J=8.2 Hz, 1H), 6.70 (s, 1H), 6.64 (d, J=8.6 Hz, 1H), 4.27 (t, J=14.1 Hz, 3H), 3.69-3.61 (m, 1H), 3.37 (s, 1H), 3.11-2.95 (m, 1H), 2.90-2.60 (m, 6H), 2.49 (t, J=10.8 Hz, 1H), 2.40 (s, 3H), 2.28-2.10 (m, 2H), 2.09-1.97 (m, 1H), 1.96-1.83 (m, 1H), 1.82-1.35 (m, 9H), 1.30-1.10 (m, 2H), 1.03 (s, 5H). LCMS: C₃₉H₄₈N₈O₃ requires: 676, found: m/z=677 [M+H]⁺.

### Example 260. 3-(6-(4-(((R)-4-(2-(6,6-dimethyl-4,5,6,7-tetrahydro-1H-indazol-3-yl)-1H-indole-6-carbonyl)-3-methylpiperazin-1-yl)methyl)piperidin-1-yl)pyridin-3-yl)piperidine-2,6-dione (132)

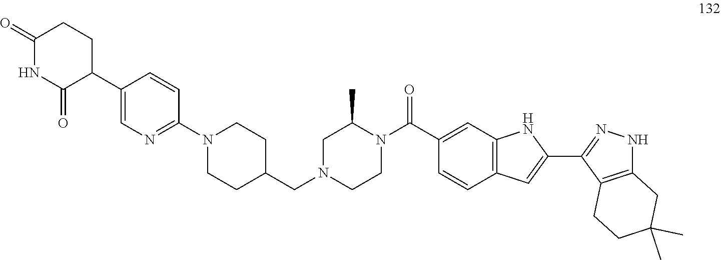 [SMILES: C[C@@H]1CN(CC2CCN(c3ccc(C4CCC(=O)NC4=O)cn3)CC2)CCN1C(=O)c1ccc2cc(-c3n[nH]c4c3CCC(C)(C)C4)[nH]c2c1]

Using general procedure 16, (R)-(2-(6,6-dimethyl-4,5,6,7-tetrahydro-1H-indazol-3-yl)-1H-indol-6-yl)(2-methylpiperazin-1-yl)methanone (20 mg, 0.051 mmol) was treated with 1-(5-(2,6-dioxopiperidin-3-yl)pyridin-2-yl)piperidine-4-carbaldehyde (HCB60) (20 mg, 0.066 mmol) to afford the title compound (10 mg, 0.010 mmol, 29%). ¹H NMR (500 MHz, CDCl₃) δ 10.89 (s, 1H), 10.76-10.60 (m, 1H), 8.04 (d, J=2.6 Hz, 1H), 7.57 (d, J=8.2 Hz, 1H), 7.51 (s, 1H), 7.29 (dq, J=8.0, 3.0, 2.5 Hz, 1H), 7.08 (d, J=8.1 Hz, 1H), 6.70 (d, J=2.0 Hz, 1H), 6.65 (dd, J=8.9, 5.2 Hz, 1H), 4.28 (ddt, J=16.7, 9.0, 3.9 Hz, 3H), 3.64 (dd, J=10.5, 5.3 Hz, 1H), 2.88-2.63 (m, 7H), 2.40 (s, 2H), 2.28-2.00 (m, 7H), 1.82 (t, J=14.9 Hz, 2H), 1.72 (t, J=8.0 Hz, 1H), 1.62 (t, J=6.5 Hz, 2H), 1.40-1.10 (m, 5H), 1.03 (s, 8H). LCMS: C₃₉H₄₈N₈O₃ requires: 676, found: m/z=677 [M+H]⁺.

### Example 261. 3-(6-(4-((4-(2-(6,6-dimethyl-4,5,6,7-tetrahydro-1H-indazol-3-yl)-1H-indole-6-carbonyl)-2,2-dimethylpiperazin-1-yl)methyl)piperidin-1-yl)pyridin-3-yl)piperidine-2,6-dione (133)

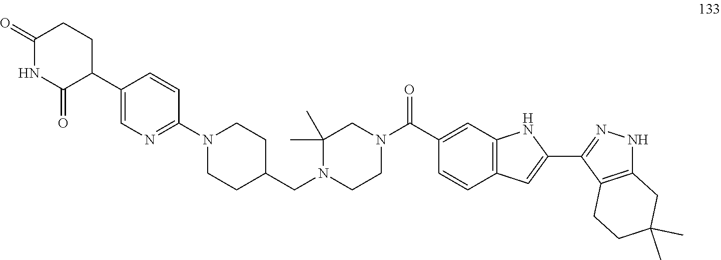 [SMILES: CC1(C)CCc2c(-c3cc4ccc(C(=O)N5CCN(CC6CCN(c7ccc(C8CCC(=O)NC8=O)cn7)CC6)C(C)(C)C5)cc4[nH]3)n[nH]c2C1]

Using general procedure 16, (2-(6,6-dimethyl-4,5,6,7-tetrahydro-1H-indazol-3-yl)-1H-indol-6-yl)(3,3-dimethylpiperazin-1-yl)methanone (30 mg, 0.074 mmol) was treated with 1-(5-(2,6-dioxopiperidin-3-yl)pyridin-2-yl)piperidine-4-carbaldehyde (25 mg, 0.081 mmol) to afford the title compound (22 mg, 0.032 mmol, 43%). ¹H NMR (500 MHz, (CD₃)₂SO) δ 12.56 (s, 1H), 11.44 (s, 1H), 10.79 (s, 1H), 7.92 (s, 1H), 7.53 (d, J=8.0 Hz, 1H), 7.44 (s, 1H), 7.34 (d, J=8.6 Hz, 1H), 6.99 (d, J=7.9 Hz, 1H), 6.77 (d, J=8.7 Hz, 1H), 6.61 (s, 1H), 4.26 (d, J=12.6 Hz, 2H), 3.71 (dd, J=11.9, 5.1 Hz, 1H), 3.52 (s, 3H), 2.81-2.61 (m, 5H), 2.54 (s, 1H), 2.42 (s, 2H), 2.16 (d, J=7.8 Hz, 3H), 1.97 (d, J=12.7 Hz, 1H), 1.77 (d, J=12.6 Hz, 2H), 1.58 (d, J=6.1 Hz, 2H), 1.23 (s, 2H), 1.10-0.70 (m, 13H). LCMS: C₄₀H₅₀N₈O₃ requires: 690, found: m/z=691 [M+H]⁺.

### Example 262. 3-(6-(4-((4-(2-(6,6-dimethyl-4,5,6,7-tetrahydro-1H-indazol-3-yl)-1H-indole-6-carbonyl)-3,3-dimethylpiperazin-1-yl)methyl)piperidin-1-yl)pyridin-3-yl)piperidine-2,6-dione (134)

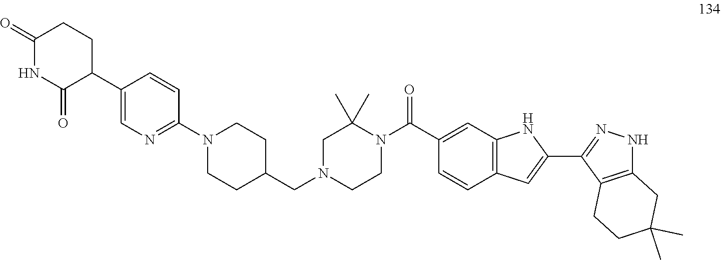 [SMILES: CC1(C)CCc2c(-c3cc4ccc(C(=O)N5CCN(CC6CCN(c7ccc(C8CCC(=O)NC8=O)cn7)CC6)CC5(C)C)cc4[nH]3)n[nH]c2C1]

Using general procedure 16, 2-(6,6-dimethyl-4,5,6,7-tetrahydro-1H-indazol-3-yl)-1H-indol-6-yl)(2,2-dimethylpiperazin-1-yl)methanone (30 mg, 0.074 mmol) was treated with 1-(5-(2,6-dioxopiperidin-3-yl)pyridin-2-yl)piperidine-4-carbaldehyde (HCB60) (25 mg, 0.081 mmol) to afford the title compound (3.8 mg, 0.005 mmol, 6.7%). LCMS: C₄₀H₅₀N₈O₃ requires: 690, found: m/z=691 [M+H]⁺.

### Example 263. 3-(4-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)phenyl)piperidine-2,6-dione (135)

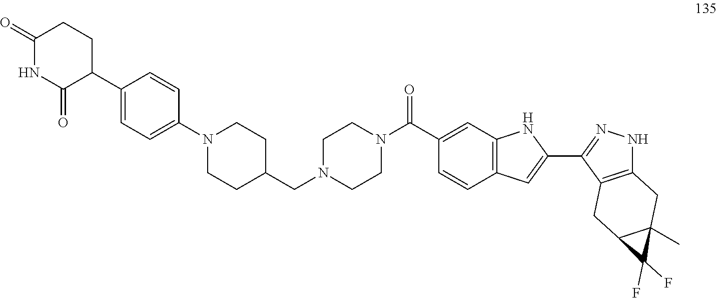 [SMILES: C[C@@]12Cc3[nH]nc(-c4cc5ccc(C(=O)N6CCN(CC7CCN(c8ccc(C9CCC(=O)NC9=O)cc8)CC7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-6-yl)(piperazin-1-yl)methanone (20 mg, 0.049 mmol) was treated with 1-(4-(2,6-dioxopiperidin-3-yl)phenyl)piperidine-4-carbaldehyde (HCB62) (16 mg, 0.054 mmol) to afford the title compound (16 mg, 0.023 mmol, 47%). ¹H NMR (500 MHz, (CD₃)₂SO) δ 12.70 (s, 1H), 11.48 (s, 1H), 10.76 (s, 1H), 7.54 (d, J=7.8 Hz, 1H), 7.44 (s, 1H), 7.02 (d, J=8.5 Hz, 3H), 6.87 (d, J=8.2 Hz, 2H), 6.62 (s, 1H), 3.71 (dd, J=10.7, 4.7 Hz, 1H), 3.65 (d, J=11.5 Hz, 2H), 3.53 (s, 4H), 3.17-2.95 (m, 2H), 2.84 (d, J=17.0 Hz, 1H), 2.62 (t, J=12.1 Hz, 3H), 2.44 (s, 1H), 2.39 (s, 5H), 2.20 (d, J=7.0 Hz, 2H), 2.12 (q, J=13.1, 12.4 Hz, 1H), 2.00 (dd, J=13.1, 6.0 Hz, 1H), 1.92-1.73 (m, 3H), 1.67 (s, 1H), 1.39 (s, 3H), 1.29-1.10 (m, 3H). LCMS: C₃₉H₄₃F₂N₇O₃ requires: 695, found: m/z=696 [M+H]⁺.

### Example 264. 3-(6-(4-((4-(2-(6,6-dimethyl-4,5,6,7-tetrahydro-1H-indazol-3-yl)-5-fluoro-1H-indole-6-carbonyl)piperazin-1-yl)methyl)piperidin-1-yl)pyridin-3-yl)piperidine-2,6-dione (136)

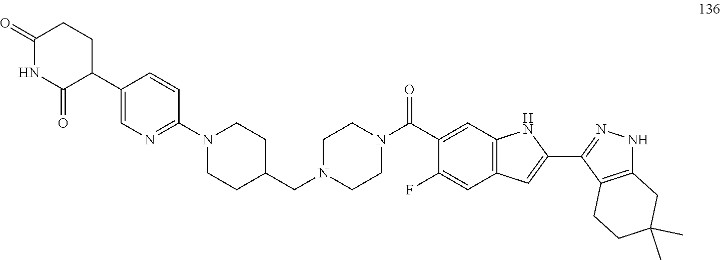 [SMILES: CC1(C)CCc2c(-c3cc4cc(F)c(C(=O)N5CCN(CC6CCN(c7ccc(C8CCC(=O)NC8=O)cn7)CC6)CC5)cc4[nH]3)n[nH]c2C1]

Using general procedure 16, (2-(6,6-dimethyl-4,5,6,7-tetrahydro-1H-indazol-3-yl)-5-fluoro-1H-indol-6-yl)(piperazin-1-yl)methanone (30 mg, 0.076 mmol) was treated with 1-(5-(2,6-dioxopiperidin-3-yl)pyridin-2-yl)piperidine-4-carbaldehyde (HCB60) (23 mg, 0.076 mmol) to afford the title compound (19 mg, 0.028 mmol, 36%). ¹H NMR (500 MHz, (CD₃)₂SO) δ 11.79 (s, 1H), 10.69 (s, 1H), 9.97 (s, 1H), 7.11 (d, J=2.5 Hz, 1H), 6.56-6.45 (m, 3H), 5.96 (d, J=8.9 Hz, 1H), 5.78 (s, 1H), 3.43 (d, J=12.7 Hz, 2H), 2.89 (dd, J=12.1, 4.9 Hz, 1H), 2.85 (s, 2H), 2.46 (s, 3H), 1.93 (t, J=12.3 Hz, 2H), 1.84 (td, J=12.0, 11.5, 5.9 Hz, 3H), 1.60 (s, 4H), 1.55 (q, J=1.9 Hz, 1H), 1.50 (s, 2H), 1.35 (dd, J=12.8, 5.1 Hz, 3H), 1.15 (dt, J=13.1, 4.4 Hz, 1H), 0.95 (d, J=11.9 Hz, 3H), 0.76 (t, J=6.4 Hz, 2H), 0.26 (q, J=12.3, 10.9 Hz, 2H), 0.19 (s, 6H). LCMS: C₃₈H₄₅FN₈O₃ requires: 680, found: m/z=681 [M+H]⁺.

### Example 265. 3-(4-(4-((4-(2-(6,6-dimethyl-4,5,6,7-tetrahydro-1H-indazol-3-yl)-5-fluoro-1H-indole-6-carbonyl)piperazin-1-yl)methyl)piperidin-1-yl)phenyl)piperidine-2,6-dione (137)

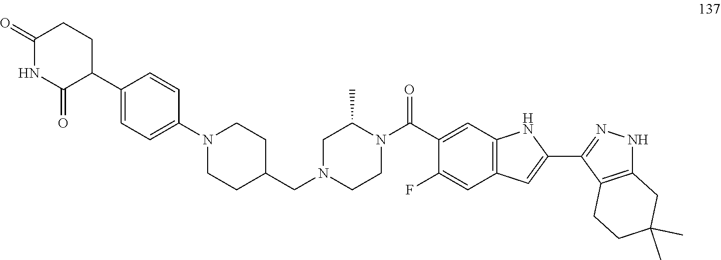 [SMILES: C[C@H]1CN(CC2CCN(c3ccc(C4CCC(=O)NC4=O)cc3)CC2)CCN1C(=O)c1cc2[nH]c(-c3n[nH]c4c3CCC(C)(C)C4)cc2cc1F]

Using general procedure 16, (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (26 mg, 0.061 mmol) was treated with 1-(4-(2,6-dioxopiperidin-3-yl)phenyl)piperidine-4-carbaldehyde (HCB62) (20 mg, 0.067 mmol) to afford the title compound (23 mg, 0.032 mmol, 52%). ¹H NMR (500 MHz, (CD₃)₂SO) δ 12.61 (s, 1H), 11.51 (s, 1H), 10.76 (s, 1H), 7.31 (dd, J=14.5, 8.0 Hz, 2H), 7.02 (d, J=8.3 Hz, 2H), 6.87 (d, J=8.4 Hz, 2H), 6.60 (s, 1H), 3.74-3.55 (m, 5H), 3.29 (d, J=13.2 Hz, 3H), 2.71-2.57 (m, 5H), 2.48-2.45 (m, 12H), 2.42 (d, J=5.4 Hz, 4H), 2.31 (s, 2H), 2.19 (d, J=7.2 Hz, 2H), 2.16-2.05 (m, 1H), 2.00 (dq, J=13.7, 5.1 Hz, 1H), 1.79 (d, J=12.7 Hz, 2H), 1.57 (t, J=6.4 Hz, 2H), 1.25-1.15 (m, 2H), 1.00 (s, 5H). LCMS: C₃₈H₄₆FN₇O₃ requires: 679, found: m/z=680 [M+H]⁺.

### Example 266. 3-(4-(4-((4-(2-((4aS,5aR)-5,5-difluoro-5a-methyl-1,4,4a,5,5a,6-hexahydrocyclopropa[f]indazol-3-yl)-5-fluoro-1H-indole-6-carbonyl)piperazin-1-yl)methyl)piperidin-1-yl)phenyl)piperidine-2,6-dione (138)

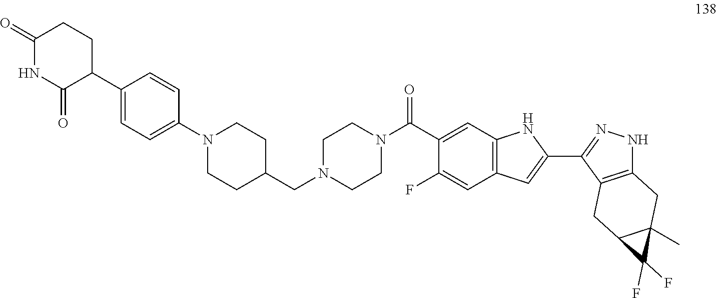 [SMILES: C[C@@]12Cc3[nH]nc(-c4cc5cc(F)c(C(=O)N6CCN(CC7CCN(c8ccc(C9CCC(=O)NC9=O)cc8)CC7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (26 mg, 0.061 mmol) was treated with 1-(4-(2,6-dioxopiperidin-3-yl)phenyl)piperidine-4-carbaldehyde (HCB62) (20 mg, 0.067 mmol) to afford the title compound (23 mg, 0.032 mmol, 52%). ¹H NMR (500 MHz, (CD₃)₂SO) δ 12.74 (s, 1H), 11.54 (s, 1H), 10.76 (s, 1H), 7.31 (dd, J=19.8, 8.1 Hz, 2H), 7.02 (d, J=8.1 Hz, 2H), 6.87 (d, J=8.2 Hz, 2H), 6.61 (s, 1H), 3.74-3.61 (m, 5H), 3.27 (s, 2H), 3.05 (dq, J=27.7, 16.4, 14.9 Hz, 3H), 2.84 (d, J=17.0 Hz, 1H), 2.61 (t, J=11.8 Hz, 3H), 2.48-2.38 (m, 2H), 2.31 (s, 2H), 2.19 (d, J=7.1 Hz, 2H), 2.16-2.06 (m, 1H), 2.00 (dd, J=13.3, 5.9 Hz, 1H), 1.90-1.82 (m, 1H), 1.79 (d, J=12.7 Hz, 2H), 1.66 (s, 1H), 1.38 (s, 3H), 1.27-1.13 (m, 2H). LCMS: C₃₉H₄₂F₃N₇O₃ requires: 713, found: m/z=714 [M+H]⁺.

### Example 267. 5-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)-N—((S)-2,6-dioxopiperidin-3-yl)picolinamide (139)

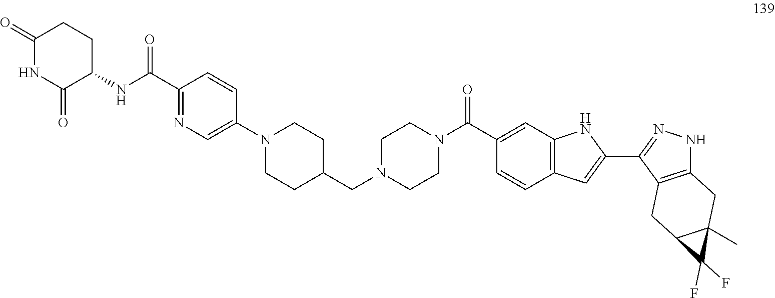 [SMILES: C[C@@]12Cc3[nH]nc(-c4cc5ccc(C(=O)N6CCN(CC7CCN(c8ccc(C(=O)N[C@H]9CCC(=O)NC9=O)nc8)CC7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-5-yl)(piperazin-1-yl)methanone (30 mg, 0.073 mmol) was treated with (S)—N-(2,6-dioxopiperidin-3-yl)-5-(4-formylpiperidin-1-yl)picolinamide (HCB28) (28 mg, 0.080 mmol) to afford the title compound (27 mg, 0.037 mmol, 51%). LCMS: C₃₉H₄₃F₂N₉O₄ requires: 739, found: m/z=740 [M+H]⁺.

### Example 268. 3-(4-(4-((4-(2-((S)-6-(methoxymethyl)-6-methyl-4,5,6,7-tetrahydro-1H-indazol-3-yl)-1H-indole-6-carbonyl)piperazin-1-yl)methyl)piperidin-1-yl)phenyl)piperidine-2,6-dione (140) (*Arbitrarily Assigned)

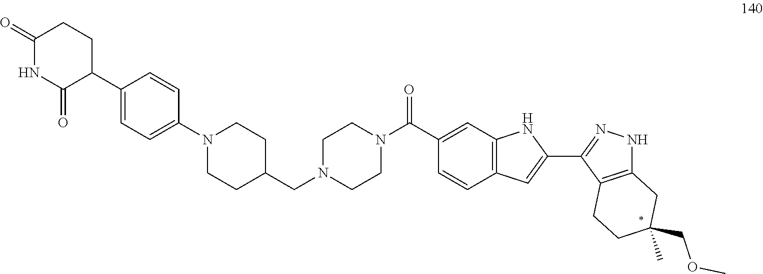 [SMILES: COC[C@@]1(C)CCc2c(-c3cc4ccc(C(=O)N5CCN(CC6CCN(c7ccc(C8CCC(=O)NC8=O)cc7)CC6)CC5)cc4[nH]3)n[nH]c2C1]

To a stirred mixture of (6S)-6-(methoxymethyl)-6-methyl-3-[6-(piperazine-1-carbonyl)-1H-indol-2-yl]-1,4,5,7-tetrahydroindazole (100 mg, 0.245 mmol, 1 equiv) in DMSO (1 mL) was added DIEA (0.5 mL) at room temperature. The resulting mixture was stirred for 15 min at room temperature. To the above mixture was added 1-[4-(2,6-dioxopiperidin-3-yl)phenyl]piperidine-4-carbaldehyde (HCB62) (73.7 mg, 0.245 mmol, 1 equiv) and HOAc (0.5 mL) at room temperature. The resulting mixture was stirred for 30 min at room temperature. To the above mixture was added NaBH(OAc)₃ (104.02 mg, 0.490 mmol, 2 equiv) at 0° C. The resulting mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The residue was purified by silica gel column chromatography, eluted with CH₃CN/H₂O (1/1) to afford (3RS)-3-(4-{4-[(4-{2-[(6S)-6-(methoxymethyl)-6-methyl-1,4,5,7-tetrahydroindazol-3-yl]-1H-indole-6-carbonyl}piperazin-1-yl)methyl]piperidin-1-yl}phenyl)piperidine-2,6-dione (36.8 mg, 20.72%) as an off-white solid. MS (ESI) calc'd for (C₄₀H₄₉N₇O₄)[M+1]⁺, 692.4; found, 692.3. ¹H NMR (400 MHz, (CD₃)₂SO) δ 12.56 (s, 1H), 11.43 (s, 1H), 10.76 (s, 1H), 7.54 (d, J=8.0 Hz, 1H), 7.45 (s, 1H), 7.04-7.02 (m, 3H), 6.99-6.89 (m, 2H), 6.62 (s, 1H), 3.73-3.67 (m, 3H), 3.54 (s, 3H), 3.29 (s, 3H), 3.19 (s, 2H), 2.68-2.58 (m, 7H), 2.46-2.31 (m, 5H), 2.31-2.22 (m, 2H), 2.22-2.19 (m, 1H), 2.10-2.01 (m, 1H), 1.82-1.71 (m, 2H), 1.79-1.70 (m, 2H), 1.69-1.60 (m, 1H), 1.24-1.20 (m, 3H), 0.98 (s, 3H). LCMS: C₄₀H₄₉N₇O₄ requires: 691, found: m/z=692 [M+H]⁺.

### Example 269. 3-(4-(4-((4-(2-((4aS,5aR)-5,5-difluoro-5a-methyl-1,4,4a,5,5a,6-hexahydrocyclopropa[f]indazol-3-yl)-1H-indole-5-carbonyl)piperazin-1-yl)methyl)-4-fluoropiperidin-1-yl)phenyl)piperidine-2,6-dione (141)

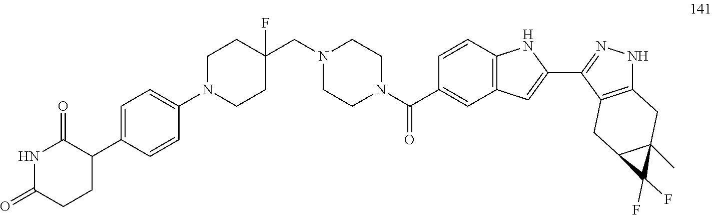 [SMILES: C[C@@]12Cc3[nH]nc(-c4cc5cc(C(=O)N6CCN(CC7(F)CCN(c8ccc(C9CCC(=O)NC9=O)cc8)CC7)CC6)ccc5[nH]4)c3C[C@@H]1C2(F)F]

Using general procedure 16, (S)-(2-(6-(methoxymethyl)-6-methyl-4,5,6,7-tetrahydro-1H-indazol-3-yl)-1H-indol-6-yl)(piperazin-1-yl)methanone (30 mg, 0.073 mmol) was treated with 1-(4-(2,6-dioxopiperidin-3-yl)phenyl)-4-fluoropiperidine-4-carbaldehyde (23 mg, 0.073 mmol) to afford the title compound (21 mg, 0.029 mmol, 40%). ¹H NMR (500 MHz, (CD₃)₂SO) δ 12.68 (s, 1H), 11.49 (s, 1H), 10.76 (s, 1H), 7.57 (s, 1H), 7.40 (d, J=8.3 Hz, 1H), 7.11 (d, J=8.3 Hz, 1H), 7.05 (d, J=8.3 Hz, 2H), 6.92 (d, J=8.7 Hz, 2H), 6.64 (s, 1H), 3.72 (dd, J=11.1, 4.9 Hz, 1H), 3.53 (s, 5H), 3.46 (d, J=12.5 Hz, 2H), 3.04 (ddd, J=39.6, 28.0, 12.9 Hz, 5H), 2.83 (d, J=16.9 Hz, 1H), 2.68-2.58 (m, 2H), 2.58-2.51 (m, 5H), 2.49-2.41 (m, 1H), 2.36 (s, 1H), 2.18-2.06 (m, 1H), 2.05-1.68 (m, 5H), 1.38 (s, 3H). LCMS: C₃₉H₄₂F₃N₇O₃ requires: 714, found: m/z=715 [M+H]⁺.

### Example 270. 5-(4-((4-(2-((4aS,5aR)-5,5-difluoro-5a-methyl-1,4,4a,5,5a,6-hexahydrocyclopropa[f]indazol-3-yl)-1H-indole-5-carbonyl)piperazin-1-yl)methyl)piperidin-1-yl)-N—((S)-2,6-dioxopiperidin-3-yl)picolinamide (142)

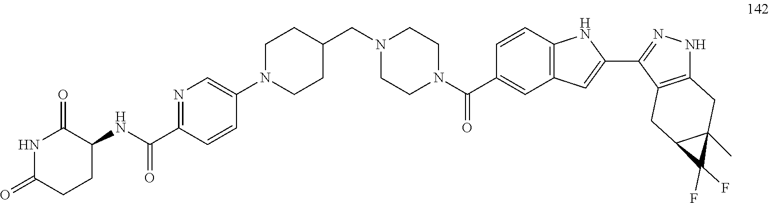 [SMILES: C[C@@]12Cc3[nH]nc(-c4cc5cc(C(=O)N6CCN(CC7CCN(c8ccc(C(=O)N[C@H]9CCC(=O)NC9=O)nc8)CC7)CC6)ccc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-5-yl)(piperazin-1-yl)methanone (30 mg, 0.073 mmol) was treated with (S)—N-(2,6-dioxopiperidin-3-yl)-5-(4-formylpiperidin-1-yl)picolinamide (HCB28) (28 mg, 0.080 mmol) to afford the title compound (33 mg, 0.042 mmol, 58%). ¹H NMR (500 MHz, (CD₃)₂SO) δ 12.69 (s, 1H), 11.49 (s, 1H), 10.84 (s, 1H), 8.69 (d, J=8.2 Hz, 1H), 8.30 (d, J=2.9 Hz, 1H), 7.57 (s, 1H), 7.40 (dt, J=9.2, 4.8 Hz, 2H), 7.11 (d, J=8.3 Hz, 1H), 6.64 (s, 1H), 4.73 (ddd, J=13.0, 8.2, 5.3 Hz, 1H), 3.93 (d, J=12.4 Hz, 2H), 3.61-3.43 (m, 5H), 3.05 (q, J=21.3, 19.4 Hz, 4H), 2.82 (dt, J=34.0, 12.8 Hz, 5H), 2.39 (s, 5H), 2.29 (d, J=6.5 Hz, OH), 2.22-2.10 (m, 4H), 2.00 (d, J=12.4 Hz, 1H), 1.81 (d, J=13.3 Hz, 5H), 1.38 (s, 4H), 1.27-1.12 (m, 3H). LCMS: C₃₉H₄₃F₂N₉O₄ requires: 740, found: m/z=741 [M+H]⁺.

### Example 271. 3-(4-((S)-3-((4-(2-((4aS,5aR)-5,5-difluoro-5a-methyl-1,4,4a,5,5a,6-hexahydrocyclopropa[f]indazol-3-yl)-1H-indole-5-carbonyl)piperazin-1-yl)methyl)pyrrolidin-1-yl)phenyl)piperidine-2,6-dione (143)

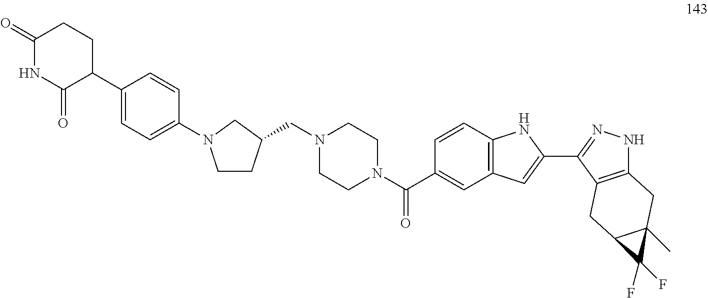 [SMILES: C[C@@]12Cc3[nH]nc(-c4cc5cc(C(=O)N6CCN(C[C@@H]7CCN(c8ccc(C9CCC(=O)NC9=O)cc8)C7)CC6)ccc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-5-yl)(piperazin-1-yl)methanone (15 mg, 0.037 mmol) was treated with (3R)-1-(4-(2,6-dioxopiperidin-3-yl)phenyl)pyrrolidine-3-carbaldehyde (11 mg, 0.040 mmol) to afford the title compound (11 mg, 0.040 mmol, 43%). ¹H NMR (500 MHz, (CD₃)₂SO) δ 12.68 (s, 1H), 11.49 (s, 1H), 10.73 (s, 1H), 7.57 (s, 1H), 7.41 (d, J=8.3 Hz, 1H), 7.11 (d, J=8.4 Hz, 1H), 6.98 (d, J=8.2 Hz, 2H), 6.64 (s, 1H), 6.48 (d, J=8.2 Hz, 2H), 3.67 (dd, J=10.7, 5.0 Hz, 1H), 3.61-3.45 (m, 4H), 3.38-3.31 (m, 1H), 3.29-3.05 (m, 3H), 3.03 (s, 1H), 2.97 (dd, J=9.5, 6.6 Hz, 1H), 2.83 (d, J=16.9 Hz, 1H), 2.60 (ddt, J=23.4, 13.1, 5.9 Hz, 2H), 2.48-2.35 (m, 3H), 2.14-2.04 (m, 2H), 1.99 (dq, J=13.6, 5.1 Hz, 1H), 1.90-1.83 (m, 1H), 1.70 (dq, J=15.2, 7.7 Hz, 1H), 1.38 (s, 3H). LCMS: C₃₈H₄₁F₂N₇O₃ requires: 682, found: m/z=683 [M+H]⁺.

### Example 272. 5-(4-((4-(2-((4aS,5aR)-5,5-difluoro-5a-methyl-1,4,4a,5,5a,6-hexahydrocyclopropa[f]indazol-3-yl)-5-fluoro-1H-indole-6-carbonyl)piperazin-1-yl)methyl)piperidin-1-yl)-N—((S)-2,6-dioxopiperidin-3-yl)picolinamide (144)

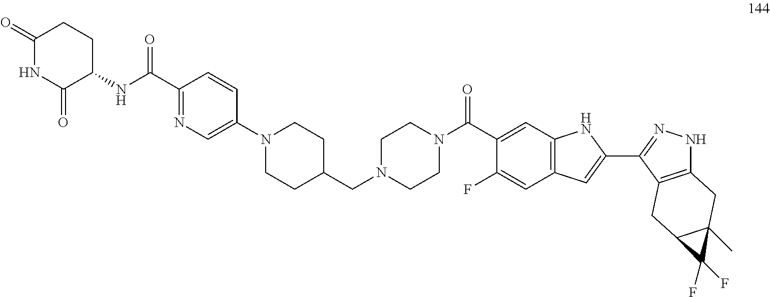 [SMILES: C[C@@]12Cc3[nH]nc(-c4cc5cc(F)c(C(=O)N6CCN(CC7CCN(c8ccc(C(=O)N[C@H]9CCC(=O)NC9=O)nc8)CC7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (15 mg, 0.035 mmol) was treated with (S)—N-(2,6-dioxopiperidin-3-yl)-5-(4-formylpiperidin-1-yl)picolinamide (HCB28) (13 mg, 0.038 mmol) to afford the title compound (20 mg, 0.027 mmol, 76%). ¹H NMR (500 MHz, (CD₃)₂SO) δ 12.74 (s, 1H), 11.54 (s, 1H), 10.83 (s, 1H), 8.68 (d, J=8.3 Hz, 1H), 8.29 (d, J=2.8 Hz, 1H), 7.83 (d, J=8.8 Hz, 1H), 7.39 (dd, J=9.0, 2.8 Hz, 1H), 7.33 (d, J=10.5 Hz, 1H), 7.29 (d, J=5.4 Hz, 1H), 6.61 (s, 1H), 4.73 (ddd, J=13.0, 8.2, 5.3 Hz, 1H), 3.92 (d, J=12.4 Hz, 2H), 3.66 (s, 2H), 3.27 (s, 2H), 3.05 (dq, J=27.1, 16.3, 14.3 Hz, 3H), 2.91-2.72 (m, 4H), 2.43 (s, 2H), 2.33 (d, J=23.3 Hz, 2H), 2.16 (dt, J=21.6, 7.6 Hz, 3H), 2.03-1.97 (m, 1H), 1.84 (td, J=27.4, 24.0, 14.0 Hz, 3H), 1.38 (s, 3H), 1.26-1.11 (m, 2H). LCMS: C₃₉H₄₂F₃N₉O₄ requires: 758, found: m/z=759 [M+H]⁺.

### Example 273. 3-(4-((R)-3-((4-(2-((4aS,5aR)-5,5-difluoro-5a-methyl-1,4,4a,5,5a,6-hexahydrocyclopropa[f]indazol-3-yl)-5-fluoro-1H-indole-6-carbonyl)piperazin-1-yl)methyl)pyrrolidin-1-yl)phenyl)piperidine-2,6-dione (145)

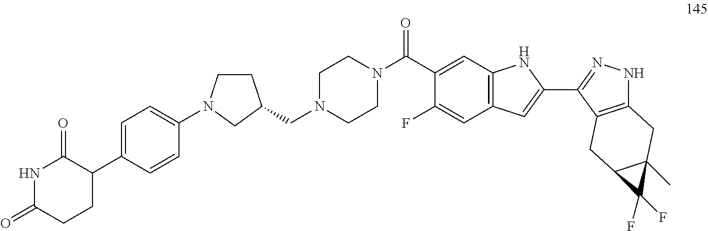 [SMILES: C[C@@]12Cc3[nH]nc(-c4cc5cc(F)c(C(=O)N6CCN(C[C@H]7CCN(c8ccc(C9CCC(=O)NC9=O)cc8)C7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (15 mg, 0.037 mmol) was treated with (3S)-1-(4-(2,6-dioxopiperidin-3-yl)phenyl)pyrrolidine-3-carbaldehyde (11 mg, 0.040 mmol) to afford the title compound (15 mg, 0.021 mmol, 57%). ¹H NMR (500 MHz, (CD₃)₂SO) δ 12.74 (s, 1H), 11.54 (s, 1H), 10.73 (s, 1H), 7.32 (dd, J=18.6, 8.1 Hz, 2H), 6.98 (d, J=8.1 Hz, 2H), 6.61 (s, 1H), 6.47 (d, J=8.2 Hz, 2H), 3.67 (dd, J=10.7, 5.1 Hz, 2H), 3.35 (d, J=7.3 Hz, 1H), 3.28 (s, 2H), 3.24-2.92 (m, 4H), 2.87-2.80 (m, 1H), 2.60 (ddt, J=22.8, 12.1, 6.1 Hz, 1H), 2.44 (dt, J=17.1, 4.7 Hz, 1H), 2.36 (d, J=7.1 Hz, 4H), 2.09 (tt, J=9.3, 5.9 Hz, 2H), 2.00 (dq, J=8.1, 5.0 Hz, 1H), 1.86 (dd, J=15.0, 7.0 Hz, 1H), 1.70 (dd, J=12.4, 7.3 Hz, 1H), 1.38 (s, 3H). LCMS: C₃₈H₄₀F₃N₇O₃ requires: 700, found: m/z=701 [M+H]⁺.

### Example 274. 3-(4-((S)-3-((4-(2-((4aS,5aR)-5,5-difluoro-5a-methyl-1,4,4a,5,5a,6-hexahydrocyclopropa[f]indazol-3-yl)-5-fluoro-1H-indole-6-carbonyl)piperazin-1-yl)methyl)pyrrolidin-1-yl)phenyl)piperidine-2,6-dione (146)

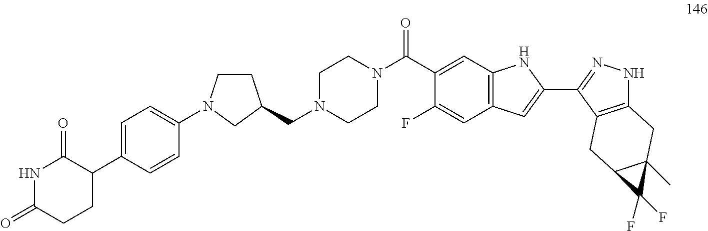 [SMILES: C[C@@]12Cc3[nH]nc(-c4cc5cc(F)c(C(=O)N6CCN(C[C@@H]7CCN(c8ccc(C9CCC(=O)NC9=O)cc8)C7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (15 mg, 0.037 mmol) was treated with (3R)-1-(4-(2,6-dioxopiperidin-3-yl)phenyl)pyrrolidine-3-carbaldehyde (11 mg, 0.040 mmol) to afford the title compound (22 mg, 0.031 mmol, 85%). ¹H NMR (500 MHz, (CD₃)₂SO) δ 12.74 (s, 1H), 11.54 (s, 1H), 10.73 (s, 1H), 7.32 (dd, J=18.6, 8.0 Hz, 2H), 6.98 (d, J=8.2 Hz, 2H), 6.61 (s, 1H), 6.47 (d, J=8.2 Hz, 2H), 3.67 (dd, J=10.7, 5.1 Hz, 2H), 3.39-3.34 (m, 1H), 3.30-3.25 (m, 2H), 3.23-2.92 (m, 4H), 2.87-2.80 (m, 1H), 2.65-2.52 (m, 1H), 2.44 (dt, J=17.2, 4.7 Hz, 1H), 2.36 (d, J=7.5 Hz, 2H), 2.08 (q, J=7.3, 5.2 Hz, 2H), 2.04-1.95 (m, 1H), 1.90-1.83 (m, 1H), 1.70 (dq, J=14.7, 7.5 Hz, 1H), 1.38 (s, 3H). LCMS: C₃₈H₄₀F₃N₇O₃ requires: 700, found: m/z=701 [M+H]⁺.

### Example 275. 3-(4-((R)-3-((4-(2-((4aS,5aR)-5,5-difluoro-5a-methyl-1,4,4a,5,5a,6-hexahydrocyclopropa[f]indazol-3-yl)-1H-indole-5-carbonyl)piperazin-1-yl)methyl)pyrrolidin-1-yl)phenyl)piperidine-2,6-dione (147)

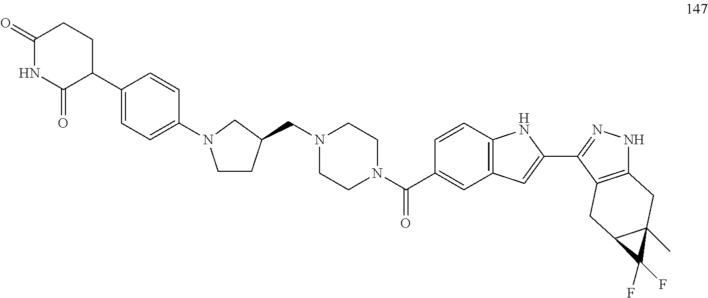 [SMILES: C[C@@]12Cc3[nH]nc(-c4cc5cc(C(=O)N6CCN(C[C@H]7CCN(c8ccc(C9CCC(=O)NC9=O)cc8)C7)CC6)ccc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-5-yl)(piperazin-1-yl)methanone (15 mg, 0.037 mmol) was treated with (3S)-1-(4-(2,6-dioxopiperidin-3-yl)phenyl)pyrrolidine-3-carbaldehyde (11 mg, 0.040 mmol) to afford the title compound (15 mg, 0.022 mmol, 61%). ¹H NMR (500 MHz, (CD₃)₂SO) δ 12.68 (s, 1H), 11.51-11.47 (m, 1H), 10.73 (s, 1H), 7.58 (s, 1H), 7.41 (d, J=8.2 Hz, 1H), 7.11 (d, J=8.4 Hz, 1H), 6.98 (d, J=8.1 Hz, 2H), 6.64 (s, 1H), 6.48 (d, J=8.2 Hz, 2H), 3.67 (dd, J=10.7, 5.0 Hz, 1H), 3.54 (s, 4H), 3.36 (d, J=9.0 Hz, 1H), 3.30-2.93 (m, 5H), 2.87-2.80 (m, 1H), 2.66-2.52 (m, 1H), 2.47-2.35 (m, 3H), 2.15-2.05 (m, 2H), 2.04-1.95 (m, 1H), 1.71 (dt, J=12.4, 7.5 Hz, 1H), 1.38 (s, 3H). LCMS: C₃₈H₄₁F₂N₇O₃ requires: 682, found: m/z=683 [M+H]⁺.

### Example 276. (S)-3-(4-(4-((4-(2-((4aS,5aR)-5,5-difluoro-5a-methyl-1,4,4a,5,5a,6-hexahydrocyclopropa[f]indazol-3-yl)-1H-indole-5-carbonyl)piperazin-1-yl)methyl)piperidin-1-yl)phenyl)piperidine-2,6-dione (148) (*Arbitrarily Assigned)

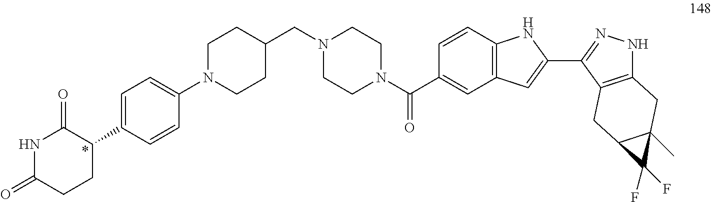 [SMILES: C[C@@]12Cc3[nH]nc(-c4cc5cc(C(=O)N6CCN(CC7CCN(c8ccc([C@@H]9CCC(=O)NC9=O)cc8)CC7)CC6)ccc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-5-yl)(piperazin-1-yl)methanone (31 mg, 0.075 mmol) was treated with (S)-1-(4-(2,6-dioxopiperidin-3-yl)phenyl)piperidine-4-carbaldehyde (HCB63) (25 mg, 0.083 mmol) to afford the title compound (24 mg, 0.034 mmol, 45%). ¹H NMR (500 MHz, (CD₃)₂SO) δ 12.61 (s, 1H), 11.47 (s, 1H), 10.76 (s, 1H), 7.57 (s, 1H), 7.41 (d, J=8.3 Hz, 1H), 7.11 (d, J=8.3 Hz, 1H), 7.02 (d, J=8.2 Hz, 2H), 6.87 (d, J=8.3 Hz, 2H), 6.65 (s, 1H), 3.71 (dd, J=11.0, 5.0 Hz, 1H), 3.65 (d, J=11.9 Hz, 2H), 3.53 (s, 4H), 3.12 (dd, J=16.6, 7.1 Hz, 1H), 3.08-2.98 (m, 2H), 2.83 (dd, J=16.5, 3.4 Hz, 1H), 2.62 (t, J=11.6 Hz, 3H), 2.48-2.40 (m, 1H), 2.38 (s, 4H), 2.20 (d, J=7.1 Hz, 2H), 2.17-2.05 (m, 1H), 2.00 (dq, J=13.9, 5.1 Hz, 1H), 1.86 (dd, J=15.0, 6.9 Hz, 1H), 1.82-1.76 (m, 2H), 1.71-1.64 (m, 1H), 1.38 (s, 3H), 1.21 (dq, J=20.6, 8.5, 6.6 Hz, 2H). LCMS: C₃₉H₄₃F₂N₇O₃ requires: 696, found: m/z=697 [M+H]⁺.

### Example 277. 5-((R)-3-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)pyrrolidin-1-yl)-2-(2,6-dioxopiperidin-3-yl)isoindoline-1,3-dione (149)

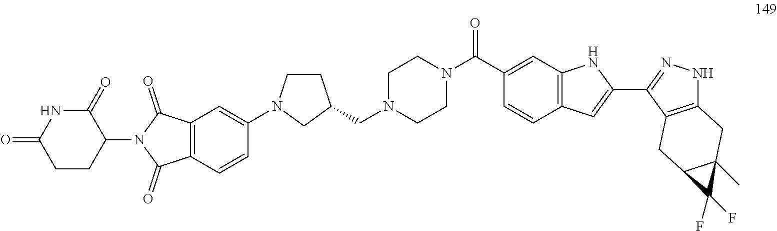 [SMILES: C[C@@]12Cc3[nH]nc(-c4cc5ccc(C(=O)N6CCN(C[C@H]7CCN(c8ccc9c(c8)C(=O)N(C8CCC(=O)NC8=O)C9=O)C7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-6-yl)(piperazin-1-yl)methanone (18 mg, 0.044 mmol) was treated with (3S)-1-(2-(2,6-dioxopiperidin-3-yl)-1,3-dioxoisoindolin-5-yl)pyrrolidine-3-carbaldehyde (HCB17) (17 mg, 0.048 mmol) to afford the title compound (14 mg, 0.018 mmol, 41%). ¹H NMR (500 MHz, (CD₃)₂SO) δ 12.70 (s, 1H), 11.48 (s, 1H), 11.06 (s, 1H), 7.64 (d, J=8.4 Hz, 1H), 7.54 (d, J=8.1 Hz, 1H), 7.45 (s, 1H), 7.01 (d, J=8.1 Hz, 1H), 6.90 (s, 1H), 6.81 (d, J=8.6 Hz, 1H), 6.63 (s, 1H), 5.05 (dd, J=12.9, 5.4 Hz, 1H), 3.53 (d, J=30.4 Hz, 5H), 3.40 (q, J=8.2 Hz, 1H), 3.20-2.96 (m, 4H), 2.93-2.79 (m, 2H), 2.67-2.53 (m, 2H), 2.48 (s, 3H), 2.46-2.34 (m, 3H), 2.14 (s, 1H), 2.00 (dd, J=9.9, 4.7 Hz, 1H), 1.38 (s, 3H). LCMS: C₄₀H₄₀F₂N₈O₅ requires: 751, found: m/z=752 [M+H]⁺.

### Example 278. 5-((R)-3-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)pyrrolidin-1-yl)-2-(2,6-dioxopiperidin-3-yl)isoindoline-1,3-dione (150)

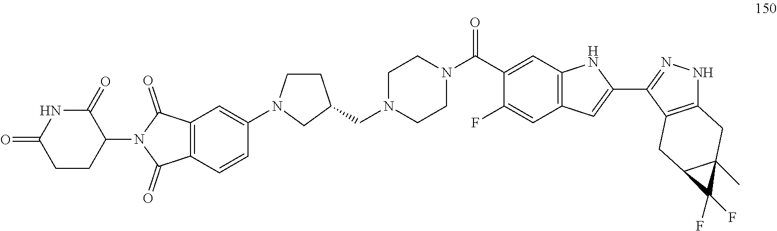 [SMILES: C[C@@]12Cc3[nH]nc(-c4cc5cc(F)c(C(=O)N6CCN(C[C@H]7CCN(c8ccc9c(c8)C(=O)N(C8CCC(=O)NC8=O)C9=O)C7)CC6)cc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-5-fluoro-1H-indol-6-yl)(piperazin-1-yl)methanone (BBX₄₅) (15 mg, 0.035 mmol) was treated with (3S)-1-(2-(2,6-dioxopiperidin-3-yl)-1,3-dioxoisoindolin-5-yl)pyrrolidine-3-carbaldehyde (14 mg, 0.039 mmol) to afford the title compound (21 mg, 0.027 mmol, 76%). ¹H NMR (500 MHz, (CD₃)₂SO) δ 12.75 (s, 1H), 11.55 (s, 1H), 11.06 (s, 1H), 7.63 (d, J=8.4 Hz, 1H), 7.37-7.29 (m, 2H), 6.90 (s, 1H), 6.81 (d, J=8.5 Hz, 1H), 6.62 (s, 1H), 5.05 (dd, J=12.9, 5.4 Hz, 1H), 3.68 (s, 2H), 3.61-3.45 (m, 2H), 3.39 (d, J=9.4 Hz, 1H), 3.33 (s, 1H), 3.29 (s, 1H), 3.13 (dd, J=17.9, 9.4 Hz, 2H), 3.09-2.96 (m, 2H), 2.93-2.80 (m, 2H), 2.67-2.53 (m, 2H), 2.39 (d, J=7.4 Hz, 2H), 2.13 (d, J=6.5 Hz, OH), 2.00 (d, J=11.9 Hz, 1H), 1.93-1.82 (m, 1H), 1.76 (t, J=10.2 Hz, 1H), 1.38 (s, 3H). LCMS: C₄₀H₃₉F₃N₈O₅ requires: 769, found: m/z=770 [M+H]⁺.

### Example 279. 5-((R)-3-((4-(2-((4aS,5aR)-5,5-difluoro-5a-methyl-1,4,4a,5,5a,6-hexahydrocyclopropa[f]indazol-3-yl)-1H-indole-5-carbonyl)piperazin-1-yl)methyl)pyrrolidin-1-yl)-2-(2,6-dioxopiperidin-3-yl)isoindoline-1,3-dione (151)

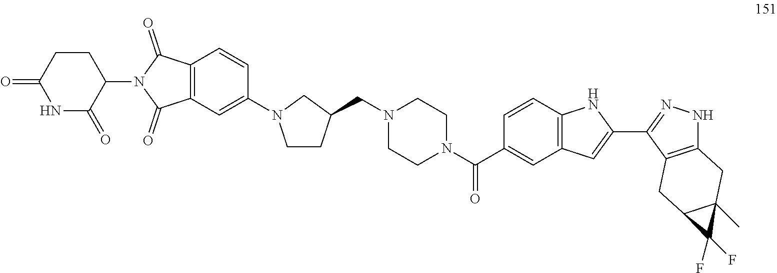 [SMILES: C[C@@]12Cc3[nH]nc(-c4cc5cc(C(=O)N6CCN(C[C@H]7CCN(c8ccc9c(c8)C(=O)N(C8CCC(=O)NC8=O)C9=O)C7)CC6)ccc5[nH]4)c3C[C@@H]1C2(F)F]

Using general procedure 16, (2-((4aS,5aR)-5,5-difluoro-5a-methyl-1,4,4a,5,5a,6-hexahydrocyclopropa[f]indazol-3-yl)-1H-indol-5-yl)(piperazin-1-yl)methanone (15 mg, 0.037 mmol) was treated with (3S)-1-(2-(2,6-dioxopiperidin-3-yl)-1,3-dioxoisoindolin-5-yl)pyrrolidine-3-carbaldehyde (15 mg, 0.041 mmol) to afford the title compound (9.6 mg, 0.013 mmol, 34%). ¹H NMR (500 MHz, (CD₃)₂SO) δ 12.68 (s, 1H), 11.49 (s, 1H), 11.06 (s, 1H), 7.64 (d, J=8.3 Hz, 1H), 7.58 (s, 1H), 7.41 (d, J=8.9 Hz, 1H), 7.14-7.09 (m, 1H), 6.90 (s, 1H), 6.81 (d, J=8.5 Hz, 1H), 6.64 (s, 1H), 5.05 (d, J=11.8 Hz, 1H), 3.61-3.53 (m, 3H), 3.52-3.48 (m, 1H), 3.40 (d, J=8.8 Hz, 1H), 3.20-2.96 (m, 3H), 2.86 (q, J=17.3, 16.3 Hz, 2H), 2.58 (dd, J=35.3, 18.0 Hz, 2H), 2.41 (p, J=18.2, 16.4 Hz, 5H), 2.19-1.96 (m, 2H), 1.91-1.70 (m, 2H), 1.38 (s, 2H). LCMS: C₄₀H₄₀F₂N₈O₅ requires: 751, found: m/z=752 [M+H]⁺.

### Example 280. (3RS)-3-[4-(4-{[(2S)-4-[2-(6,6-dimethyl-4,5,6,7-tetrahydro-1H-indazol-3-yl)-1H-indole-6-carbonyl]-2-methylpiperazin-1-yl]methyl}piperidin-1-yl)phenyl]piperidine-2,6-dione (152)

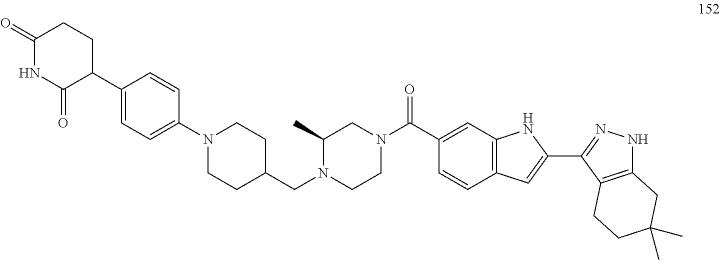 [SMILES: C[C@H]1CN(C(=O)c2ccc3cc(-c4n[nH]c5c4CCC(C)(C)C5)[nH]c3c2)CCN1CC1CCN(c2ccc(C3CCC(=O)NC3=O)cc2)CC1]

This compound was synthesized following the general procedure 16. LCMS: C₄₀H₄₉N₇O₃ requires: 675.4, found: m/z=676.7 [M+H]⁺.

### Example 281. (3RS)-3-[4-(4-{[(2S)-4-{2-[(4aS,5aR)-5,5-difluoro-5a-methyl-1H,4H,4aH,5H,5aH,6H-cyclopropa[f]indazol-3-yl]-1H-indole-6-carbonyl}-2-methylpiperazin-1-yl]methyl}piperidin-1-yl)phenyl]piperidine-2,6-dione (153)

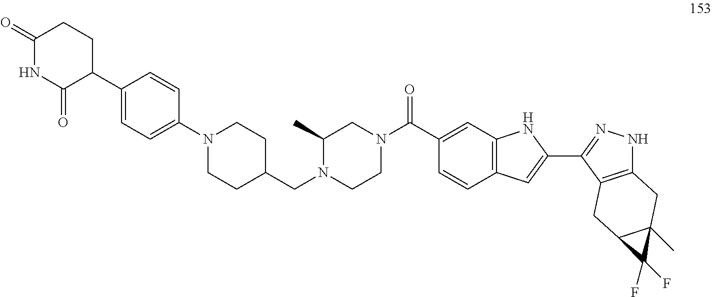 [SMILES: C[C@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)cc2)CC1]

This compound was synthesized following the general procedure 16. LCMS: C₄₀H₄₅F₂N₇O₃ requires: 709.4, found: m/z=710.7 [M+H]⁺.

### Example 282. (3RS)-3-(4-{4-[(3-{2-[(4aS,5aR)-5,5-difluoro-5a-methyl-1H,4H,4aH,5H,5aH,6H-cyclopropa[f]indazol-3-yl]-1H-indole-6-carbonyl}-3,8-diazabicyclo[3.2.1]octan-8-yl)methyl]piperidin-1-yl}phenyl)piperidine-2,6-dione (154)

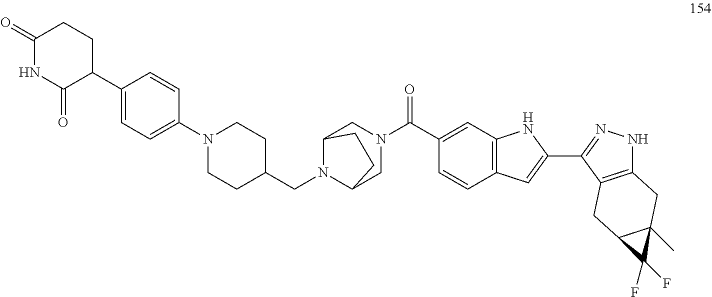 [SMILES: C[C@@]12Cc3[nH]nc(-c4cc5ccc(C(=O)N6CC7CCC(C6)N7CC6CCN(c7ccc(C8CCC(=O)NC8=O)cc7)CC6)cc5[nH]4)c3C[C@@H]1C2(F)F]

This compound was synthesized following the general procedure 16. LCMS: C₄₁H₄₅F₂N₇O₃ requires: 721.4, found: m/z=722.7 [M+H]⁺.

### Example 283. (3RS)-3-[4-(4-{[(2S)-4-{2-[(4aS,5aR)-5,5-difluoro-5a-methyl-1H,4H,4aH,5H,5aH,6H-cyclopropa[f]indazol-3-yl]-1H-indole-6-carbonyl}-2-methylpiperazin-1-yl]methyl}piperidin-1-yl)-3-fluorophenyl]piperidine-2,6-dione (155)

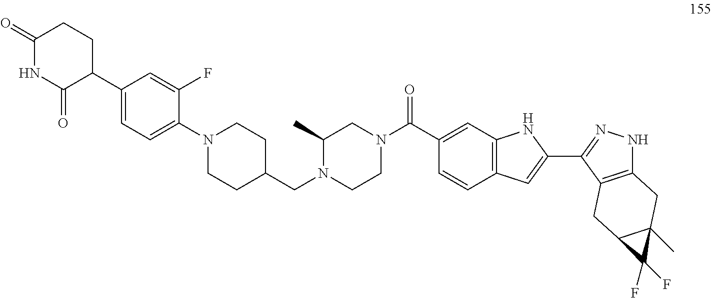 [SMILES: C[C@H]1CN(C(=O)c2ccc3cc(-c4n[nH]c5c4C[C@@H]4C(F)(F)[C@]4(C)C5)[nH]c3c2)CCN1CC1CCN(c2ccc(C3CCC(=O)NC3=O)cc2F)CC1]

This compound was synthesized following the general procedure 16. LCMS: C₄₀H₄₄F₃N₇O₃ requires: 727.3, found: m/z=728.7 [M+H]⁺.

### Example 284. 3-[4-(4-{[(2R)-4-[2-(6,6-Dimethyl-1,4,5,7-tetrahydroindazol-3-yl)-1H-indole-6-carbonyl]-2-methylpiperazin-1-yl]methyl}piperidin-1-yl)phenyl]piperidine-2,6-dione (156)

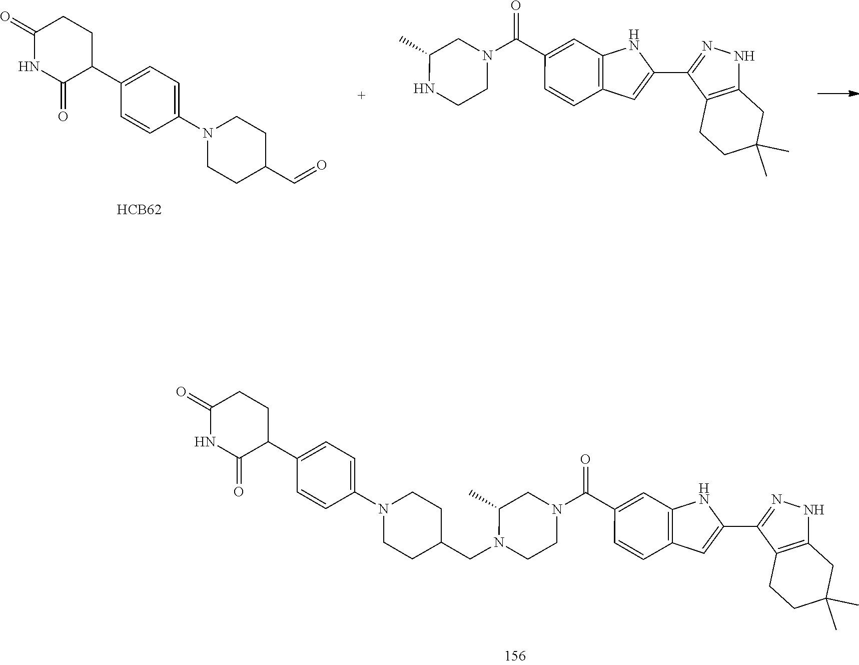

To a solution of rac-1-{4-2,6-dioxopiperidin-3-yl]phenyl}piperidine-4-carbaldehyde (HCB62) (27 mg, 0.0899 mmol) and (R)-(2-(6,6-dimethyl-4,5,6,7-tetrahydro-1H-indazol-3-yl)-1H-indol-6-yl)(3-methylpiperazin-1-yl)methanone (35.2 mg, 0.0899 mmol) and N,N-diisopropylethylamine (62 ul, 0.3596 mmol) in DCM, sodium triacetoxyborohydride (57.16 mg, 0.27 mmol) was added, stirred for 1 hour. The crude product was quenched with H₂O, extract with DCM twice. The organic layer was collected, dried over Na₂SO₄ and concentrated. ISCO silica gel column purification eluted with MeOH in DCM (0-12%) to afford 3-[4-(4-{[(2R)-4-[2-(6,6-dimethyl-1,4,5,7-tetrahydroindazol-3-yl)-1H-indole-6-carbonyl]-2-methylpiperazin-1-yl]methyl}piperidin-1-yl)phenyl]piperidine-2,6-dione (156) (26 mg, 42.8%) LCMS C₄₀H₄₉N₇O₃ required: 675.4 found m/z=676.5 [M+H]+¹H NMR (500 MHz, DMSO) δ 12.60 (s, 1H), 11.54 (s, 1H), 10.78 (s, 1H), 7.69-7.44 (m, 2H), 7.08 (dd, J=21.2, 8.1 Hz, 3H), 6.92 (d, J=8.3 Hz, 2H), 6.65 (s, 1H), 3.73 (dd, J=11.1, 5.0 Hz, 2H), 3.37 (d, J=25.2 Hz, 17H), 2.84-2.57 (m, 5H), 2.43 (s, 2H), 2.21-2.06 (m, 1H), 2.06-1.81 (m, 3H), 1.80-1.67 (m, 1H), 1.59 (t, J=6.4 Hz, 2H), 1.30 (d, J=48.6 Hz, 8H), 1.02 (s, 6H).

### General Procedure 17: Synthesis of (158) 3-(4-(4-(((2R,6S)-4-(2-(6,6-dimethyl-4,5,6,7-tetrahydro-1H-indazol-3-yl)-1H-indole-6-carbonyl)-2,6-dimethylpiperazin-1-yl)methyl)piperidin-1-yl)phenyl)piperidine-2,6-dione (157) and 3-(4-(4-(((2R,6S)-4-(2-((4aS,5aR)-5,5-difluoro-5a-methyl-1,4,4a,5,5a,6-hexahydrocyclopropa[f]indazol-3-yl)-1H-indole-6-carbonyl)-2,6-dimethylpiperazin-1-yl)methyl)piperidin-1-yl)phenyl)piperidine-2,6-dione (158)

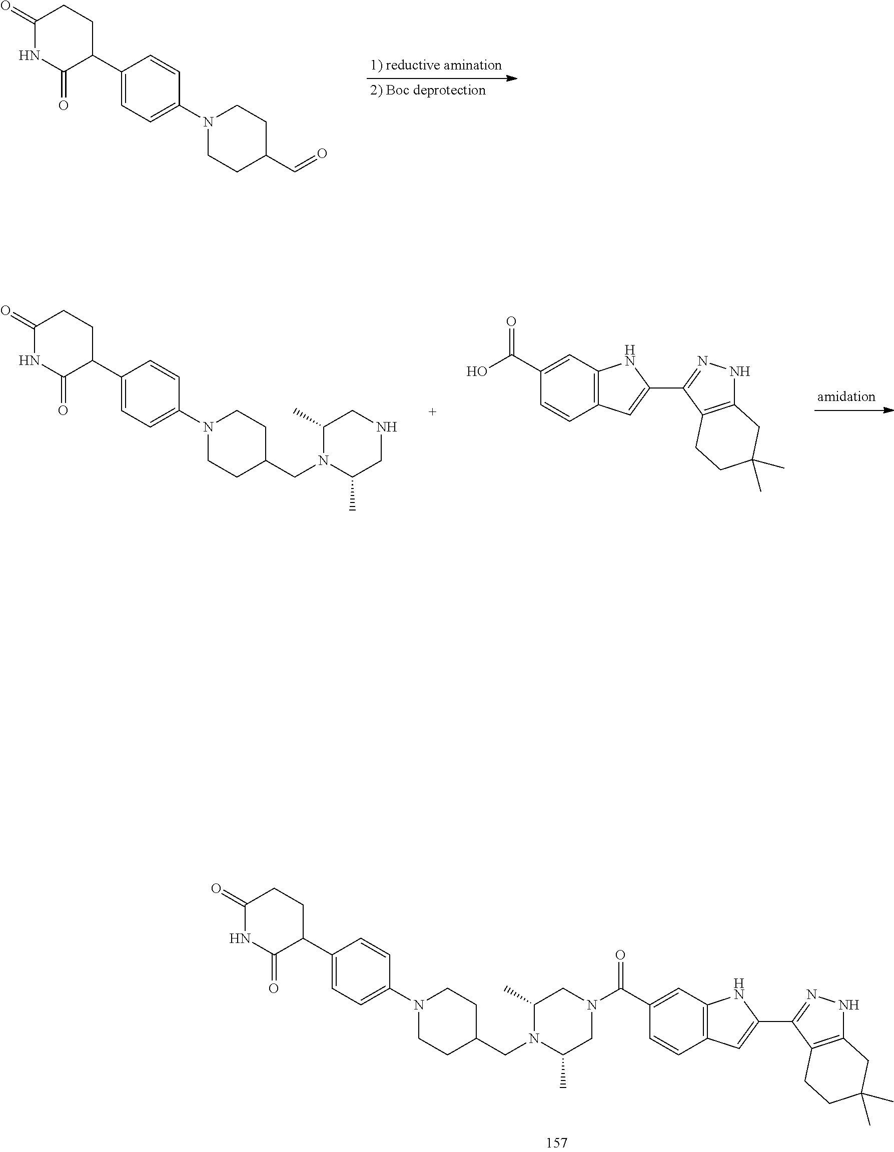 [SMILES: O=CC1CCN(c2ccc(C3CCC(=O)NC3=O)cc2)CC1 ; C[C@@H]1CNC[C@H](C)N1CC1CCN(c2ccc(C3CCC(=O)NC3=O)cc2)CC1 ; CC1(C)CCc2c(-c3cc4ccc(C(=O)O)cc4[nH]3)n[nH]c2C1 ; C[C@@H]1CN(C(=O)c2ccc3cc(-c4n[nH]c5c4CCC(C)(C)C5)[nH]c3c2)C[C@H](C)N1CC1CCN(c2ccc(C3CCC(=O)NC3=O)cc2)CC1]

### Scheme 17: Synthesis of Compound 157

### Example 285. 3-(4-(4-(((2R,6S)-4-(2-(6,6-dimethyl-4,5,6,7-tetrahydro-1H-indazol-3-yl)-1H-indole-6-carbonyl)-2,6-dimethylpiperazin-1-yl)methyl)piperidin-1-yl)phenyl)piperidine-2,6-dione (157)

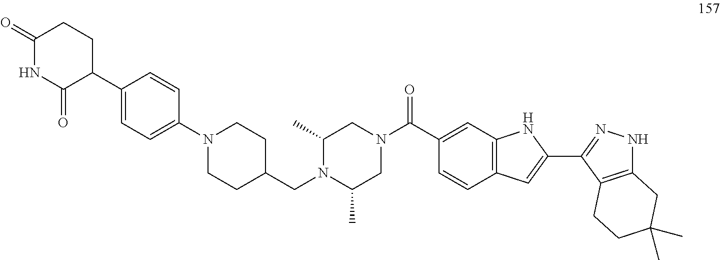 [SMILES: C[C@@H]1CN(C(=O)c2ccc3cc(-c4n[nH]c5c4CCC(C)(C)C5)[nH]c3c2)C[C@H](C)N1CC1CCN(c2ccc(C3CCC(=O)NC3=O)cc2)CC1]

A mixture of amine (34 mg, 0.087 mmol), acid (27 mg, 0.087 mmol), BOP (42 mg, 0.095 mmol), i-Pr₂NEt (75 μL, 0.43 mmol) in DMF (450 μL) was allowed to stir at rt for 16 hr. The reaction was quenched with H₂O. The reaction mixture was extracted with EtOAc, washed with saturated aqueous NaCl, dried over Na₂SO₄, and concentrated under reduced pressure. PTLC (SiO₂, 7.5% MeOH/DCM) provided the title compound (29 mg, 0.041 mmol, 47%). ¹H NMR (500 MHz, (CD₃)₂SO) δ 12.57 (s, 1H), 11.45 (s, 1H), 10.76 (s, 1H), 7.53 (d, J=8.1 Hz, 1H), 7.45 (s, 1H), 7.02 (d, J=8.4 Hz, 3H), 6.87 (d, J=8.3 Hz, 2H), 6.61 (s, 1H), 3.74-3.64 (m, 3H), 2.71-2.53 (m, 4H), 2.50 (p, J=1.8 Hz, 9H), 2.48-2.33 (m, 4H), 2.11 (ddt, J=15.5, 11.3, 5.8 Hz, 1H), 2.00 (dq, J=13.6, 5.0 Hz, 1H), 1.82 (d, J=12.4 Hz, 2H), 1.58 (t, J=6.4 Hz, 2H), 1.20 (dt, J=24.2, 10.6 Hz, 2H), 1.13 (s, 15H). LCMS: C₄₁H₅₁N₇O₃ requires: 690, found: m/z=691 [M+H]⁺.

### Example 286. 3-(4-(4-(((2R,6S)-4-(2-((4aS,5aR)-5,5-difluoro-5a-methyl-1,4,4a,5,5a,6-hexahydrocyclopropa[f]indazol-3-yl)-1H-indole-6-carbonyl)-2,6-dimethylpiperazin-1-yl)methyl)piperidin-1-yl)phenyl)piperidine-2,6-dione (158)

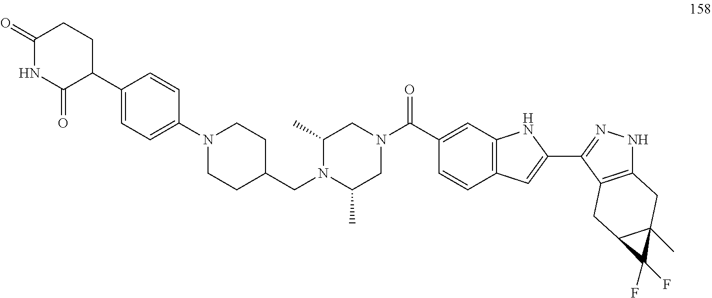 [SMILES: C[C@@H]1CN(C(=O)c2ccc3cc(-c4n[nH]c5c4C[C@@H]4C(F)(F)[C@]4(C)C5)[nH]c3c2)C[C@H](C)N1CC1CCN(c2ccc(C3CCC(=O)NC3=O)cc2)CC1]

Using general procedure 17, 3-(4-(4-(((2R,6S)-2,6-dimethylpiperazin-1-yl)methyl)piperidin-1-yl)phenyl)piperidine-2,6-dione (34 mg, 0.085 mmol) was treated with 2-((4aS,5aR)-5,5-difluoro-5a-methyl-1,4,4a,5,5a,6-hexahydrocyclopropa[f]indazol-3-yl)-1H-indole-6-carboxylic acid (29 mg, 0.085 mmol) to afford the title compound (30 mg, 0.040 mmol, 47%). ¹H NMR (500 MHz, (CD₃)₂SO) δ 12.70 (s, 1H), 11.48 (s, 1H), 10.76 (s, 1H), 7.54 (d, J=8.1 Hz, 1H), 7.45 (s, 1H), 7.02 (d, J=8.2 Hz, 3H), 6.87 (d, J=8.4 Hz, 2H), 6.63 (s, 1H), 3.74-3.63 (m, 3H), 3.05 (q, J=22.1, 19.1 Hz, 3H), 2.90-2.70 (m, 1H), 2.60 (ddd, J=25.8, 12.4, 8.1 Hz, 2H), 2.48-2.42 (m, 1H), 2.38 (d, J=6.6 Hz, 2H), 2.11 (ddt, J=16.3, 12.0, 5.9 Hz, 1H), 2.00 (dq, J=13.6, 5.1 Hz, 1H), 1.86 (dd, J=33.2, 16.5 Hz, 3H), 1.49 (s, 1H), 1.39 (s, 3H), 1.25-1.14 (m, 3H), 1.01 (s, 7H). LCMS: C₄₁H₄₇F₂N₇O₃ requires: 724, found: m/z=725 [M+H]⁺.

### Example 287. 3-(4-(4-(((R)-4-(2-((4aS,5aR)-5,5-difluoro-5a-methyl-1,4,4a,5,5a,6-hexahydrocyclopropa[f]indazol-3-yl)-1H-indole-6-carbonyl)-2-methylpiperazin-1-yl)methyl)piperidin-1-yl)phenyl)piperidine-2,6-dione (159)

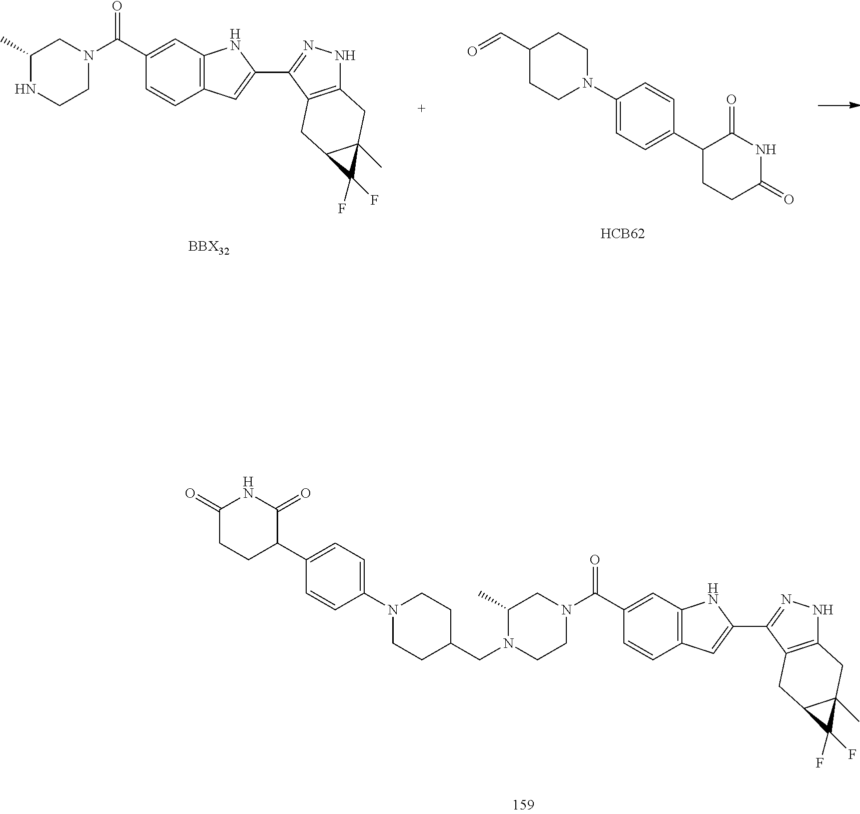

To a mixture of (2-((4aS,5aR)-5,5-difluoro-5a-methyl-1,4,4a,5,5a,6-hexahydrocyclopropa[f]indazol-3-yl)-1H-indol-6-yl)((R)-3-methylpiperazin-1-yl)methanone (BBX₃₂) (37 mg, 0.09 mmol) and 1-(4-(2,6-dioxopiperidin-3-yl)phenyl)piperidine-4-carbaldehyde (HCB62) (26 mg, 0.09 mmol) in DCM (2.00 mL) was added N,N-diisopropylethylamine (0.06 mL, 0.04 g, 0.35 mmol). The mixture was sonicated. Sodium triacetoxyborohydride (55 mg, 0.26 mmol) was added. After 1 hour, water was added. The mixture was extracted three times with DCM. The combined organic layers were concentrated. The crude residue was purified by preparative TLC eluted with 10% MeOH/DCM to provide 3-(4-(4-(((R)-4-(2-((4aS,5aR)-5,5-difluoro-5a-methyl-1,4,4a,5,5a,6-hexahydrocyclopropa[f]indazol-3-yl)-1H-indole-6-carbonyl)-2-methylpiperazin-1-yl)methyl)piperidin-1-yl)phenyl)piperidine-2,6-dione (0.0249 g, 39.5%). ¹H NMR (500 MHz, DMSO) δ 12.71 (s, 1H), 11.49 (s, 1H), 10.77 (s, 1H), 7.55 (d, J=8.1 Hz, 1H), 7.45 (s, 1H), 7.06-6.99 (m, 3H), 6.91-6.86 (m, 2H), 6.63 (s, 1H), 3.72 (dd, J=11.0, 4.9 Hz, 1H), 3.66 (d, J=11.8 Hz, 3H), 3.20-2.98 (m, 4H), 2.89-2.82 (m, 2H), 2.69-2.58 (m, 4H), 2.50-2.36 (m, 2H), 2.23-1.97 (m, 5H), 1.88 (d, J=13.6 Hz, 2H), 1.74 (d, J=12.6 Hz, 1H), 1.64-1.61 (m, 1H), 1.40 (s, 3H), 1.29-1.15 (m, 2H), 0.98-0.95 (m, 4H). LCSM C₄₀H₄₅F₂N₇O₃ requires: 709, found: m/z=710 [M+H]⁺.

### Example 288. 3-(4-(4-(((R)-4-(2-((4aS,5aR)-5,5-difluoro-5a-methyl-1,4,4a,5,5a,6-hexahydrocyclopropa[f]indazol-3-yl)-1H-indole-6-carbonyl)-2-methylpiperazin-1-yl)methyl)piperidin-1-yl)-2-fluorophenyl)piperidine-2,6-dione (160)

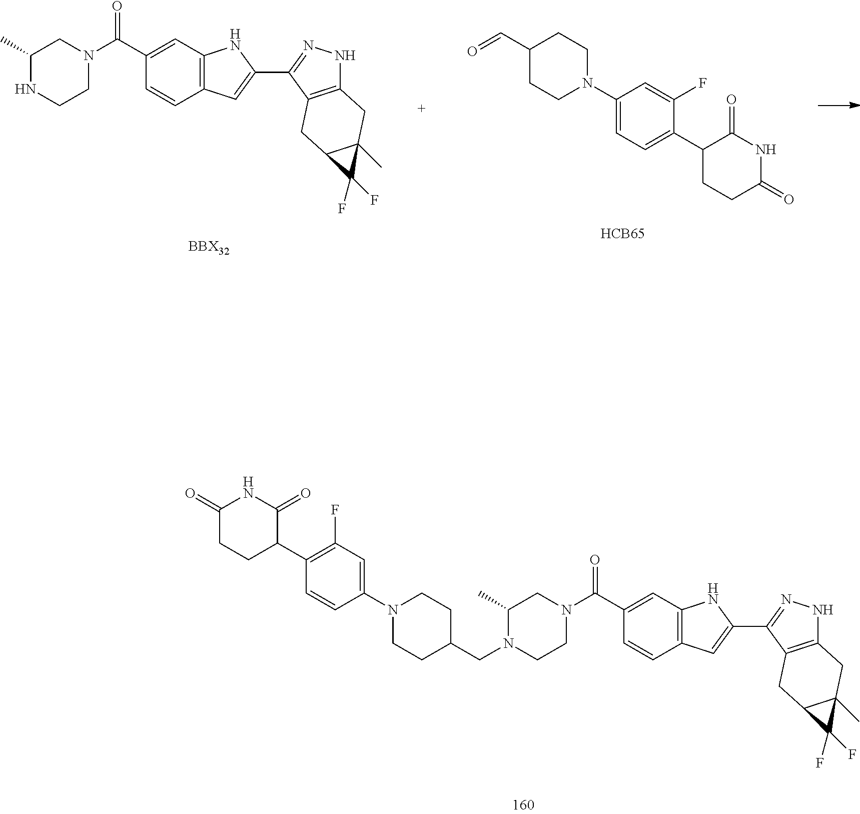

To a mixture of (2-((4aS,5aR)-5,5-difluoro-5a-methyl-1,4,4a,5,5a,6-hexahydrocyclopropa[f]indazol-3-yl)-1H-indol-6-yl)((R)-3-methylpiperazin-1-yl)methanone (BBX₃₂) (38 mg, 0.09 mmol) and N,N-diisopropylethylamine (0.06 mL, 0.05 g, 0.36 mmol) in DCM (2.00 mL) was added 1-(4-(2,6-dioxopiperidin-3-yl)-3-fluorophenyl)piperidine-4-carbaldehyde (HCB65) (28 mg, 0.09 mmol) followed by sodium triacetoxyborohydride (57 mg, 0.27 mmol). After 1 hour, water was added and the mixture was extracted 3× DCM. The combined organic layers were concentrated. The crude residue was purified by preparative TLC eluted with 10% MeOH/DCM to provide 3-(4-(4-(((R)-4-(2-((4aS,5aR)-5,5-difluoro-5a-methyl-1,4,4a,5,5a,6-hexahydrocyclopropa[f]indazol-3-yl)-1H-indole-6-carbonyl)-2-methylpiperazin-1-yl)methyl)piperidin-1-yl)-2-fluorophenyl)piperidine-2,6-dione (160) (0.0234 g, 34.6%). ¹HNMR (500 MHz, DMSO) δ 12.73 (s, 1H), 11.49 (s, 1H), 10.80 (s, 1H), 7.55 (d, J=8.0 Hz, 1H), 7.46 (s, 1H), 7.07 (t, J=8.7 Hz, 1H), 7.02 (d, J=8.1 Hz, 1H), 6.74-6.67 (m, 2H), 6.64 (s, 1H), 3.88 (dd, J=12.4, 5.0 Hz, 2H), 3.75-3.69 (m, 2H), 3.16-3.11 (m, 1H), 3.10-3.00 (m, 3H), 2.88-2.81 (m, 3H), 2.77-2.65 (m, 3H), 2.43-2.40 (m, 2H), 2.23-2.01 (m, 4H), 2.00-1.91 (m, 1H), 1.86 (d, J=13.3 Hz, 2H), 1.77-1.60 (m, 2H), 1.40 (s, 3H), 1.26-1.09 (m, 2H), 0.98-0.94 (m, 4H). LCSM C₄₀H₄₄F₃N₇O₃ requires: 727, found: m/z=728 [M+H]⁺.

### Example 289. 3-(4-(4-(((R)-4-(2-((4aS,5aR)-5,5-difluoro-5a-methyl-1,4,4a,5,5a,6-hexahydrocyclopropa[f]indazol-3-yl)-1H-indole-6-carbonyl)-2-methylpiperazin-1-yl)methyl)piperidin-1-yl)-3-fluorophenyl)piperidine-2,6-dione (161)

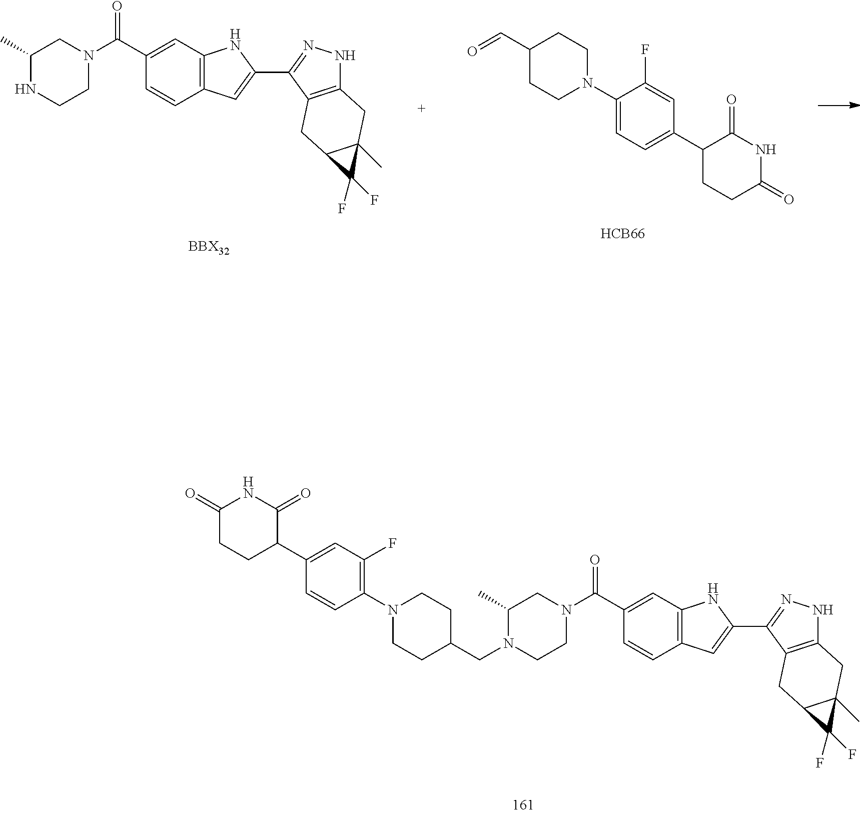

To a mixture of (2-((4aS,5aR)-5,5-difluoro-5a-methyl-1,4,4a,5,5a,6-hexahydrocyclopropa[f]indazol-3-yl)-1H-indol-6-yl)((R)-3-methylpiperazin-1-yl)methanone (BBX₃₂) (37 mg, 0.09 mmol) and N,N-diisopropylethylamine (0.06 mL, 0.04 g, 0.35 mmol) in DCM (2.00 mL) was added 1-(4-(2,6-dioxopiperidin-3-yl)-2-fluorophenyl)piperidine-4-carbaldehyde (HCB66) (28 mg, 0.09 mmol) followed by sodium triacetoxyborohydride (55 mg, 0.26 mmol). After 1 hour, water was added. The mixture was extracted three times with DCM. The combined organic layers were concentrated. The crude residue was purified by preparative TLC eluted with 10% MeOH/DCM to provide 3-(4-(4-(((R)-4-(2-((4aS,5aR)-5,5-difluoro-5a-methyl-1,4,4a,5,5a,6-hexahydrocyclopropa[f]indazol-3-yl)-1H-indole-6-carbonyl)-2-methylpiperazin-1-yl)methyl)piperidin-1-yl)-3-fluorophenyl)piperidine-2,6-dione (161) (0.0231 g, 35.0%). ¹H NMR (500 MHz, DMSO) δ 12.74 (s, 1H), 11.49 (s, 1H), 10.81 (s, 1H), 7.55 (d, J=8.1 Hz, 1H), 7.46 (s, 1H), 7.04-6.92 (m, 4H), 6.64 (s, 1H), 4.01-3.55 (m, 4H), 3.17-3.11 (m, 1H), 3.06-3.03 (m, 3H), 2.88-2.81 (m, 2H), 2.71-2.54 (m, 3H), 2.44-2.41 (m, 1H), 2.25-2.14 (m, 2H), 2.11-1.96 (m, 2H), 1.90-1.86 (m, 3H), 1.75 (d, J=12.6 Hz, 1H), 1.69 (s, 1H), 1.62-1.59 (m, 1H), 1.40 (s, 3H), 1.36-1.21 (m, 3H), 0.99-0.96 (m, 4H). LCSM C₄₀H₄₄F₃N₇O₃ requires: 727, found: m/z=728 [M+H]⁺.

### General Procedure 18: Chiral Resolution

### Example 290. (S)-3-(4-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)phenyl)piperidine-2,6-dione (162) (*Arbitrarily Assigned)

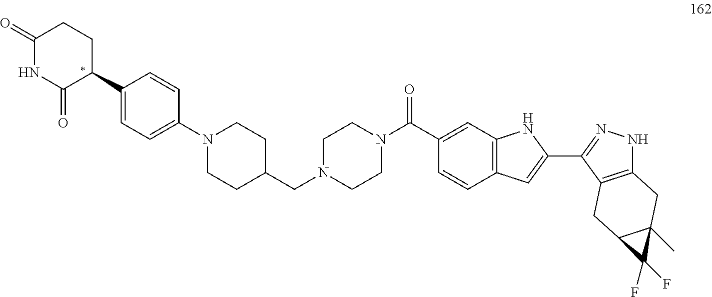 [SMILES: C[C@@]12Cc3[nH]nc(-c4cc5ccc(C(=O)N6CCN(CC7CCN(c8ccc([C@@H]9CCC(=O)NC9=O)cc8)CC7)CC6)cc5[nH]4)c3C[C@@H]1C2(F)F]

3-(4-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)phenyl)piperidine-2,6-dione were separated by SFC to provide the title compound (74 mg) and diastereomer below.

### (R)-3-(4-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)phenyl)piperidine-2,6-dione (163) (*Arbitrarily Assigned)

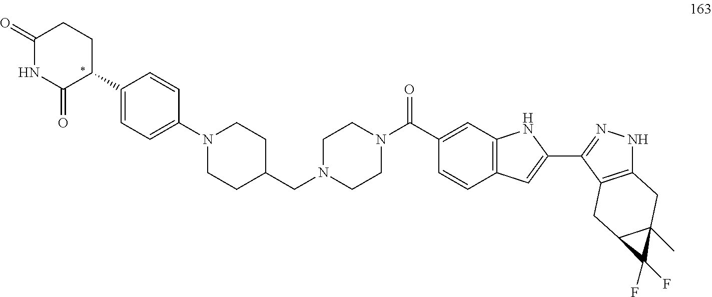 [SMILES: C[C@@]12Cc3[nH]nc(-c4cc5ccc(C(=O)N6CCN(CC7CCN(c8ccc([C@H]9CCC(=O)NC9=O)cc8)CC7)CC6)cc5[nH]4)c3C[C@@H]1C2(F)F]

(80 mg)

SFC conditions: Regis—(R,R)—Whelk-O 1, (5 μm Kromasil—4.6×150 mm), i-PrOH: MeCN (4:6)+0.1% NH₄OH, 6 ml/min, 100 bars, 40° C.

### Example 291. (4aS,5aR)-5,5-difluoro-5a-methyl-N-(1-{1-[(1rs&,4rs&)-4-({5-[(3RS)-2,6-dioxopiperidin-3-yl]-1H-indazol-1-yl}methyl)cyclohexanecarbonyl]piperidin-4-yl}-1H-pyrazol-4-yl)-1H,4H,4aH,5H,5aH,6H-cyclopropa[f]indazole-3-carboxamide (164)

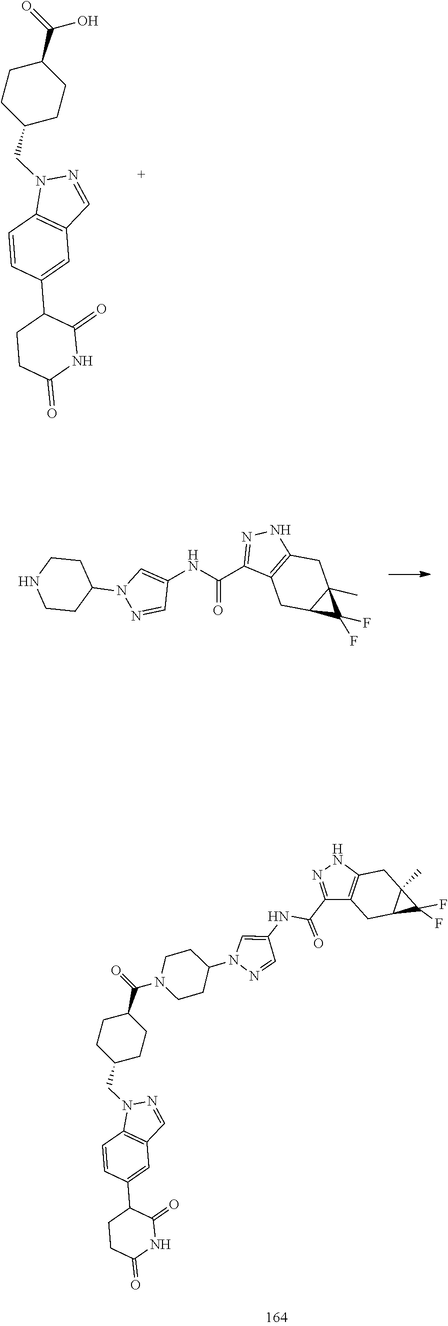 [SMILES: O=C1CCC(c2ccc3c(cnn3C[C@H]3CC[C@H](C(=O)O)CC3)c2)C(=O)N1 ; C[C@@]12Cc3[nH]nc(C(=O)Nc4cnn(C5CCNCC5)c4)c3C[C@@H]1C2(F)F ; C[C@@]12Cc3[nH]nc(C(=O)Nc4cnn(C5CCN(C(=O)[C@H]6CC[C@H](Cn7ncc8cc(C9CCC(=O)NC9=O)ccc87)CC6)CC5)c4)c3C[C@@H]1C2(F)F]

### General Procedure 19: Standard Conditions and Procedure for Amide Couplings

(1rs&,4rs&)-4-({5-[(3RS)-2,6-dioxopiperidin-3-yl]indazol-1-yl}methyl)cyclohexane-1-carboxylic acid (20 mg, 0.05 mmol), (4aS,5aR)-5,5-difluoro-5a-methyl-N-[1-(piperidin-4-yl)pyrazol-4-yl]-1H,4H,4aH,6H-cyclopropa[f]indazole-3-carboxamide (20 mg, 1 eq) and HATU (20 mg, 1 eq, dissolved in 0.1 M DMF) were combined in DIEA/DMF (0.25:1, 0.2 M) and stirred for 18 h. The reaction solution was pushed through a syringe filter and purified by ref phase HPLC to provide desired product (22 mg, 56%).LCMS: C₃₈H₄₃F₂N₉O₄ requires: 727.3, found: m/z=728.5 [M+H]⁺. ¹H NMR (500 MHz, DMSO-d₆) δ 12.96 (s, 1H), 10.85 (s, 1H), 10.12 (s, 1H), 8.03 (s, 2H), 7.69-7.52 (m, 3H), 7.25 (dd, J=8.7, 1.6 Hz, 1H), 4.51-4.30 (m, 2H), 4.27 (d, J=6.9 Hz, 2H), 4.06-3.90 (m, 2H), 3.06 (d, J=8.2 Hz, 2H), 3.02 (s, 1H), 2.83 (d, J=18.1 Hz, 1H), 2.78-2.60 (m, 3H), 2.28 (qd, J=12.3, 4.4 Hz, 1H), 2.09 (dq, J=8.0, 4.6 Hz, 1H), 2.06-1.94 (m, 2H), 1.90 (s, 1H), 1.83-1.73 (m, 2H), 1.67 (s, 3H), 1.63-1.52 (m, 2H), 1.36 (s, 4H), 1.32-1.24 (m, 2H), 1.17 (t, J=12.8 Hz, 2H).

### Example 292. (4aS,5aR)-5,5-difluoro-5a-methyl-N-(1-{1-[(1rs&,4rs&)-4-({5-[(3RS)-2,6-dioxopiperidin-3-yl]-2H-indazol-2-yl}methyl)cyclohexanecarbonyl]piperidin-4-yl}-1H-pyrazol-4-yl)-1H,4H,4aH,5H,5aH,6H-cyclopropa[f]indazole-3-carboxamide (165)

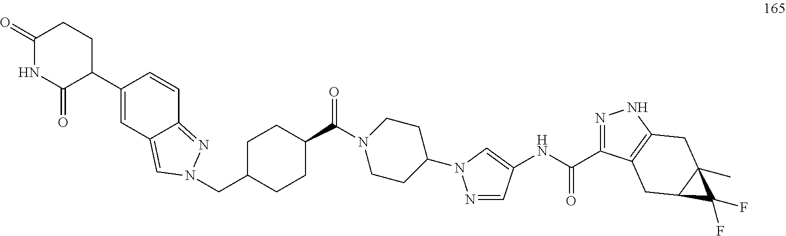

This compound was synthesized following the standard amide coupling conditions and procedure 19. LCMS: C₃₈H₄₃F₂N₉O₄ requires: 727.3, found: m/z=728.4 [M+H]⁺. ¹H NMR (500 MHz, DMSO-d₆) δ 12.96 (s, 1H), 10.84 (s, 1H), 10.12 (s, 1H), 8.31 (s, 1H), 8.03 (s, 1H), 7.63 (s, 1H), 7.56 (d, J=8.9 Hz, 1H), 7.51 (s, 1H), 7.09 (d, J=8.8 Hz, 1H), 4.46 (d, J=13.0 Hz, 1H), 4.41-4.31 (m, 1H), 4.28 (d, J=6.9 Hz, 2H), 4.03 (d, J=13.3 Hz, 1H), 3.91 (dd, J=11.4, 4.9 Hz, 1H), 3.16 (s, 2H), 3.07 (d, J=8.6 Hz, 2H), 3.02 (s, 1H), 2.83 (dd, J=17.0, 3.3 Hz, 1H), 2.70 (td, J=11.6, 5.4 Hz, 3H), 2.58 (s, 1H), 2.26 (qd, J=12.0, 4.4 Hz, 1H), 2.13-1.88 (m, 5H), 1.79 (dd, J=14.8, 6.9 Hz, 2H), 1.68 (d, J=13.2 Hz, 3H), 1.60 (q, J=15.3, 14.4 Hz, 3H), 1.36 (s, 5H), 1.14 (q, J=12.9, 12.3 Hz, 2H).

### Example 293. (4aS,5aR)-N-(1-{1-[2-(1-{5-[(3RS)-2,6-dioxopiperidin-3-yl]pyridin-2-yl}piperidin-4-yl)acetyl]piperidin-4-yl}-1H-pyrazol-4-yl)-5,5-difluoro-5a-methyl-1H,4H,4aH,5H,5aH,6H-cyclopropa[f]indazole-3-carboxamide (166)

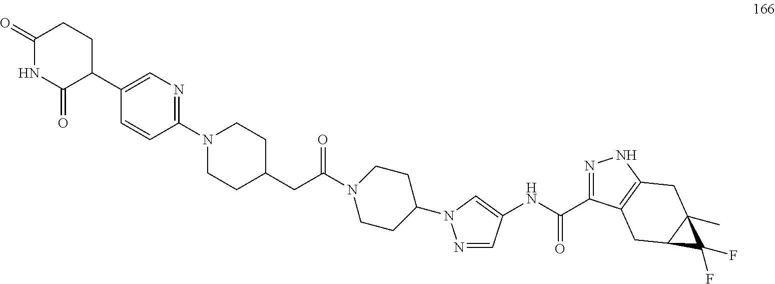 [SMILES: C[C@@]12Cc3[nH]nc(C(=O)Nc4cnn(C5CCN(C(=O)CC6CCN(c7ccc(C8CCC(=O)NC8=O)cn7)CC6)CC5)c4)c3C[C@@H]1C2(F)F]

This compound was synthesized following the standard amide coupling conditions and procedure 19. LCMS: C₃₅H₄₁F₂N₉O₄ requires: 689.3, found: m/z=690.4 [M+H]⁺. ¹H NMR (500 MHz, DMSO-d₆) δ 12.98 (s, 1H), 10.94 (s, 1H), 10.14 (s, 1H), 8.05 (s, 1H), 7.87 (d, J=2.2 Hz, 2H), 7.64 (s, 1H), 7.34 (s, 1H), 4.51 (d, J=13.0 Hz, 1H), 4.46-4.34 (m, 1H), 4.18 (d, J=13.3 Hz, 3H), 4.01 (d, J=13.9 Hz, 2H), 3.17 (s, 6H), 3.10-2.97 (m, 5H), 2.88-2.76 (m, 2H), 2.76-2.61 (m, 3H), 2.60 (s, 1H), 2.38-2.31 (m, 3H), 2.29 (dd, J=13.8, 5.2 Hz, 2H), 2.08 (s, 2H), 2.05-1.91 (m, 4H), 1.91-1.74 (m, 6H), 1.70 (dd, J=11.5, 4.2 Hz, 2H), 1.36 (s, 4H), 1.27 (d, J=19.3 Hz, 4H).

### Example 294. (4aS,5aR)-N-(1-{1-[(3RS)-1-{5-[(3RS&)-2,6-dioxopiperidin-3-yl]pyridin-2-yl}pyrrolidine-3-carbonyl]piperidin-4-yl}-1H-pyrazol-4-yl)-5,5-difluoro-5a-methyl-1H,4H,4aH,5H,5aH,6H-cyclopropa[f]indazole-3-carboxamide (167)

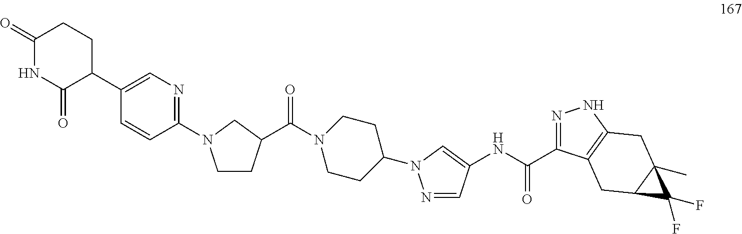 [SMILES: C[C@@]12Cc3[nH]nc(C(=O)Nc4cnn(C5CCN(C(=O)C6CCN(c7ccc(C8CCC(=O)NC8=O)cn7)C6)CC5)c4)c3C[C@@H]1C2(F)F]

This compound was synthesized following the standard amide coupling conditions and procedure 19. LCMS: C₃₃H₃₇F₂N₉O₄ requires: 661.3, found: m/z=662.4 [M+H]⁺. ¹H NMR (500 MHz, DMSO-d₆) δ 12.98 (s, 1H), 10.96 (s, 1H), 10.16 (s, 1H), 8.08 (d, J=6.2 Hz, 1H), 7.94 (s, 1H), 7.86 (s, 1H), 7.65 (s, 1H), 7.17 (d, J=25.0 Hz, 1H), 4.54-4.36 (m, 2H), 4.14 (d, J=13.8 Hz, 1H), 3.95 (dd, J=12.8, 4.8 Hz, 2H), 3.28 (s, 3H), 3.16-2.99 (m, 4H), 2.93-2.76 (m, 2H), 2.76-2.55 (m, 2H), 2.33 (dt, J=12.7, 6.1 Hz, 3H), 2.22-1.87 (m, 6H), 1.77 (t, J=16.7 Hz, 3H), 1.37 (s, 3H), 1.25 (s, 1H).

### Example 295. (4aS,5aR)-5,5-difluoro-5a-methyl-N-(1-{1-[(1r,4r)-4-({5-[(3RS)-2,6-dioxopiperidin-3-yl]pyridin-2-yl}oxy)cyclohexanecarbonyl]piperidin-4-yl}-1H-pyrazol-4-yl)-1H,4H,4aH,5H,5aH,6H-cyclopropa[f]indazole-3-carboxamide (168)

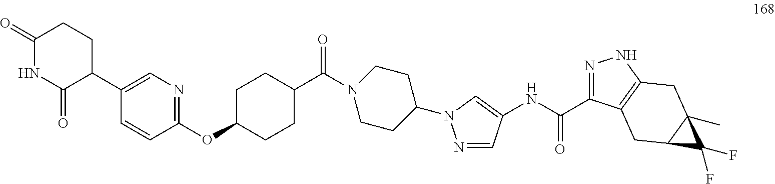

This compound was synthesized following the standard amide coupling conditions and procedure 19. LCMS: C₃₅H₄₀F₂N₈O₅ requires: 690.3, found: m/z=691.3 [M+H]⁺. ¹H NMR (500 MHz, DMSO-d₆) δ 12.96 (s, 1H), 10.86 (s, 1H), 10.13 (s, 1H), 8.12-7.92 (m, 2H), 7.64 (s, 1H), 7.56 (dd, J=8.6, 2.5 Hz, 1H), 6.75 (d, J=8.5 Hz, 1H), 4.92 (dt, J=10.6, 5.4 Hz, 1H), 4.53-4.26 (m, 2H), 4.10 (d, J=13.5 Hz, 1H), 3.85 (dd, J=12.5, 4.8 Hz, 2H), 3.14-2.95 (m, 4H), 2.83 (dd, J=17.0, 3.3 Hz, 2H), 2.71 (ddt, J=17.8, 12.4, 5.2 Hz, 4H), 2.24 (qd, J=12.9, 4.6 Hz, 2H), 2.17-2.10 (m, 2H), 2.06 (s, 1H), 2.01 (dq, J=8.2, 3.9 Hz, 2H), 1.87-1.73 (m, 4H), 1.69 (d, J=13.5 Hz, 1H), 1.54 (dt, J=20.8, 10.9 Hz, 5H), 1.36 (s, 3H), 1.25 (s, 1H).

### Example 296. (4aS,5aR)-5,5-difluoro-5a-methyl-N-(1-{1-[(1r,4r)-4-({1-[(3RS)-2,6-dioxopiperidin-3-yl]-3-methyl-2-oxo-2,3-dihydro-1H-1,3-benzodiazol-5-yl}amino)cyclohexanecarbonyl]piperidin-4-yl}-1H-pyrazol-4-yl)-1H,4H,4aH,5H,5aH,6H-cyclopropa[f]indazole-3-carboxamide (169)

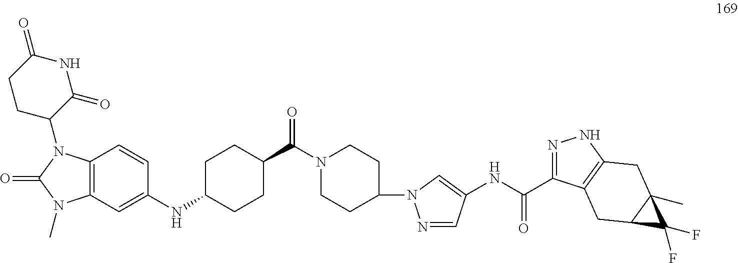 [SMILES: Cn1c(=O)n(C2CCC(=O)NC2=O)c2ccc(N[C@H]3CC[C@H](C(=O)N4CCC(n5cc(NC(=O)c6n[nH]c7c6C[C@@H]6C(F)(F)[C@]6(C)C7)cn5)CC4)CC3)cc21]

This compound was synthesized following the standard amide coupling conditions and procedure 19. LCMS: C₃₈H₄₄F₂N₁₀O₅ requires: 758.3, found: m/z=759.3 [M+H]⁺. ¹H NMR (500 MHz, DMSO-d₆) δ 12.97 (s, 1H), 11.13 (s, 1H), 10.14 (s, 1H), 8.05 (s, 1H), 7.64 (s, 1H), 5.39 (s, 1H), 4.45 (dd, J=34.1, 12.0 Hz, 3H), 4.07 (d, J=12.9 Hz, 3H), 3.19 (t, J=12.8 Hz, 2H), 3.07 (d, J=8.0 Hz, 3H), 3.03 (s, 1H), 2.90 (d, J=18.2 Hz, 1H), 2.83 (d, J=16.9 Hz, 1H), 2.70 (s, 5H), 2.09 (s, 2H), 2.02 (d, J=24.9 Hz, 5H), 1.80 (s, 4H), 1.68 (s, 1H), 1.48 (s, 3H), 1.36 (s, 3H), 1.25 (s, 1H).

### Example 297. (4aS,5aR)-N-[1-(1-{1-[(3RS)-2,6-dioxopiperidin-3-yl]-3-methyl-2-oxo-2,3-dihydro-1H-1,3-benzodiazole-5-carbonyl}piperidin-4-yl)-1H-pyrazol-4-yl]-5,5-difluoro-5a-methyl-1H,4H,4aH,5H,5aH,6H-cyclopropa[f]indazole-3-carboxamide (170)

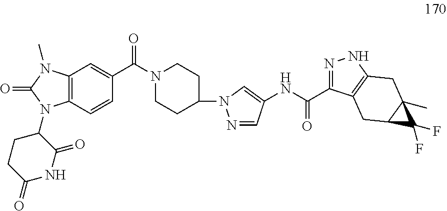 [SMILES: Cn1c(=O)n(C2CCC(=O)NC2=O)c2ccc(C(=O)N3CCC(n4cc(NC(=O)c5n[nH]c6c5C[C@@H]5C(F)(F)[C@]5(C)C6)cn4)CC3)cc21]

This compound was synthesized following the standard amide coupling conditions and procedure 19. LCMS: C₃₂H₃₃F₂N₉O₅ requires: 661.3, found: m/z=662.4 [M+H]⁺. ¹H NMR (500 MHz, DMSO-d₆) δ 12.97 (s, 1H), 11.14 (s, 1H), 10.14 (s, 1H), 8.09 (s, 1H), 7.65 (s, 1H), 7.33 (d, J=1.5 Hz, 1H), 7.26-7.07 (m, 2H), 5.43 (dd, J=12.9, 5.4 Hz, 1H), 4.44 (t, J=11.3 Hz, 2H), 3.12-2.94 (m, 4H), 2.94-2.86 (m, 1H), 2.86-2.58 (m, 4H), 2.06 (d, J=21.3 Hz, 3H), 1.90 (d, J=11.9 Hz, 2H), 1.84-1.70 (m, 1H), 1.36 (s, 3H), 1.25 (s, 1H).

### Example 298. (4aS,5aR)-N-{1-[1-(1-{1-[(3RS)-2,6-dioxopiperidin-3-yl]-3-methyl-2-oxo-2,3-dihydro-1H-1,3-benzodiazol-4-yl}piperidine-4-carbonyl)piperidin-4-yl]-1H-pyrazol-4-yl}-5,5-difluoro-5a-methyl-1H,4H,4aH,5H,5aH,6H-cyclopropa[f]indazole-3-carboxamide (171)

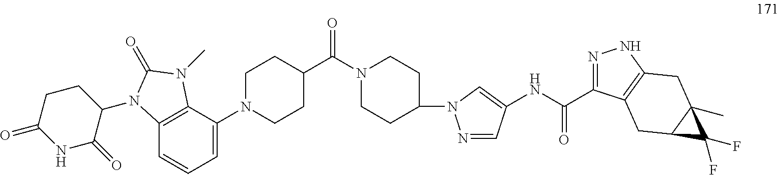 [SMILES: Cn1c(=O)n(C2CCC(=O)NC2=O)c2cccc(N3CCC(C(=O)N4CCC(n5cc(NC(=O)c6n[nH]c7c6C[C@@H]6C(F)(F)[C@]6(C)C7)cn5)CC4)CC3)c21]

This compound was synthesized following the standard amide coupling conditions and procedure 19. LCMS: C₃₇H₄₂F₂N₁₀O₅ requires: 744.3, found: m/z=745.4 [M+H]⁺. ¹H NMR (500 MHz, DMSO-d₆) δ 12.96 (s, 1H), 11.10 (s, 1H), 10.14 (s, 1H), 8.06 (s, 1H), 7.65 (s, 1H), 7.00 (t, J=8.0 Hz, 1H), 6.92-6.83 (m, 2H), 5.43-5.31 (m, 1H), 4.47 (d, J=47.8 Hz, 2H), 4.15 (s, 1H), 3.65 (s, 4H), 3.14 (s, 3H), 3.11-2.95 (m, 4H), 2.83 (d, J=15.0 Hz, 4H), 2.73 (d, J=14.2 Hz, 2H), 2.09 (s, 4H), 2.02 (s, 2H), 1.95-1.60 (m, 7H), 1.36 (s, 3H), 1.25 (s, 1H).

### Example 299. (4aS,5aR)-N-{1-[(1-{1-[4-(2,4-dioxo-1,3-diazinan-1-yl)phenyl]piperidine-4-carbonyl}piperidin-4-yl)methyl]-1H-pyrazol-4-yl}-5,5-difluoro-5a-methyl-1H,4H,4aH,5H,5aH,6H-cyclopropa[f]indazole-3-carboxamide (172)

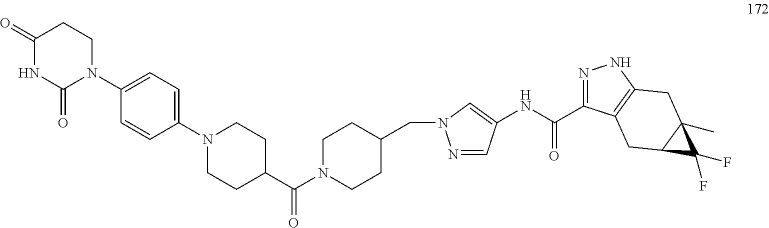 [SMILES: C[C@@]12Cc3[nH]nc(C(=O)Nc4cnn(CC5CCN(C(=O)C6CCN(c7ccc(N8CCC(=O)NC8=O)cc7)CC6)CC5)c4)c3C[C@@H]1C2(F)F]

This compound was synthesized following the standard amide coupling conditions and procedure 19. LCMS: C₃₅H₄₁F₂N₉O₄ requires: 689.3, found: m/z=690.4 [M+H]⁺.

### Example 300. (4aS,5aR)-N-(1-{[1-(1-{4-[(3RS)-2,6-dioxopiperidin-3-yl]phenyl}piperidine-4-carbonyl)piperidin-4-yl]methyl}-1H-pyrazol-4-yl)-5,5-difluoro-5a-methyl-1H,4H,4aH,5H,5aH,6H-cyclopropa[f]indazole-3-carboxamide (173)

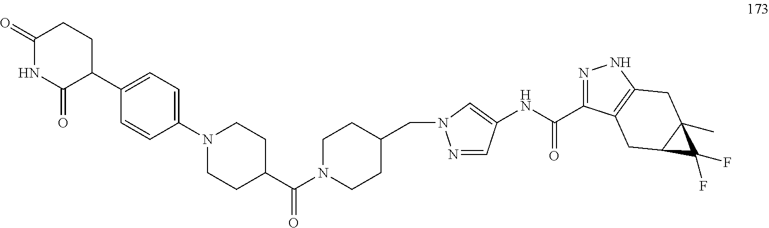 [SMILES: C[C@@]12Cc3[nH]nc(C(=O)Nc4cnn(CC5CCN(C(=O)C6CCN(c7ccc(C8CCC(=O)NC8=O)cc7)CC6)CC5)c4)c3C[C@@H]1C2(F)F]

This compound was synthesized following the standard amide coupling conditions and procedure 19. LCMS: C₃₆H₄₂F₂N₈O₄ requires: 688.3, found: m/z=689.3 [M+H]⁺.

### Example 301. 6,6-dimethyl-N-{1-[(1r,4r)-4-{6-[(3RS)-2,6-dioxopiperidin-3-yl]-1,2,3,4-tetrahydroisoquinoline-2-carbonyl}cyclohexyl]-1H-pyrazol-4-yl}-4,5,6,7-tetrahydro-1H-indazole-3-carboxamide (174)

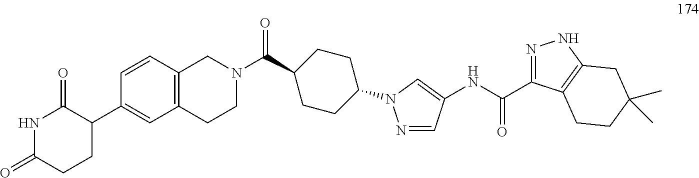 [SMILES: CC1(C)CCc2c(C(=O)Nc3cnn([C@H]4CC[C@H](C(=O)N5CCc6cc(C7CCC(=O)NC7=O)ccc6C5)CC4)c3)n[nH]c2C1]

This compound was synthesized following the standard amide coupling conditions and procedure 19. LCMS: C₃₄H₄₁N₇O₄ requires: 611.3, found: m/z=612.7 [M+H]⁺.

### Example 302. 6,6-dimethyl-N-{1-[(1r,4r)-4-(4-{5-[(3RS)-2,6-dioxopiperidin-3-yl]pyridin-2-yl}piperazine-1-carbonyl)cyclohexyl]-1H-pyrazol-4-yl}-4,5,6,7-tetrahydro-1H-indazole-3-carboxamide (175)

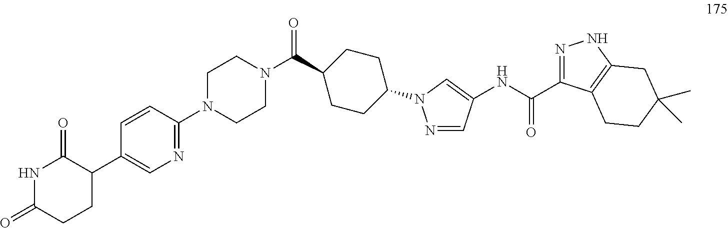 [SMILES: CC1(C)CCc2c(C(=O)Nc3cnn([C@H]4CC[C@H](C(=O)N5CCN(c6ccc(C7CCC(=O)NC7=O)cn6)CC5)CC4)c3)n[nH]c2C1]

This compound was synthesized following the standard amide coupling conditions and procedure 19. LCMS: C₃₄H₄₃N₉O₄ requires: 641.3, found: m/z=642.7 [M+H]⁺.

### Example 303. 6,6-dimethyl-N-{1-[(1r,4r)-4-(4-{4-[(3RS)-2,6-dioxopiperidin-3-yl]phenyl}piperazine-1-carbonyl)cyclohexyl]-1H-pyrazol-4-yl}-4,5,6,7-tetrahydro-1H-indazole-3-carboxamide (176)

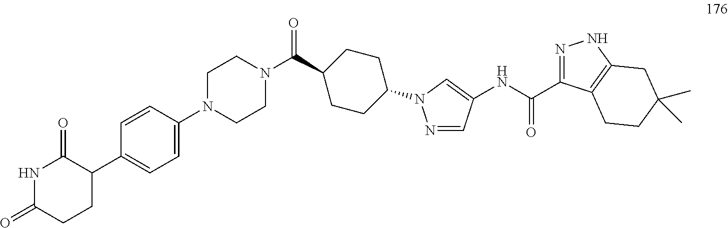 [SMILES: CC1(C)CCc2c(C(=O)Nc3cnn([C@H]4CC[C@H](C(=O)N5CCN(c6ccc(C7CCC(=O)NC7=O)cc6)CC5)CC4)c3)n[nH]c2C1]

This compound was synthesized following the standard amide coupling conditions and procedure 19. LCMS: C₃₅H₄₄N₈O₄ requires: 640.3, found: m/z=641.4 [M+H]⁺.

### Example 304. 6,6-dimethyl-N-{1-[(1r,4r)-4-[4-({5-[(3R)-2,6-dioxopiperidin-3-yl]pyridin-2-yl}oxy)piperidine-1-carbonyl]cyclohexyl]-1H-pyrazol-4-yl}-4,5,6,7-tetrahydro-1H-indazole-3-carboxamide (177)

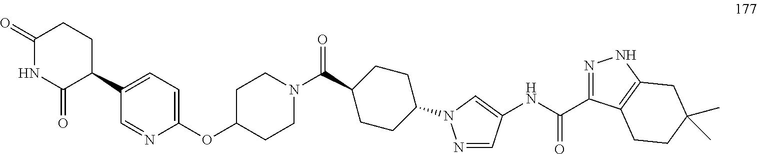 [SMILES: CC1(C)CCc2c(C(=O)Nc3cnn([C@H]4CC[C@H](C(=O)N5CCC(Oc6ccc([C@@H]7CCC(=O)NC7=O)cn6)CC5)CC4)c3)n[nH]c2C1]

This compound was synthesized following the standard amide coupling conditions and procedure 19. LCMS: C₃₅H₄₄N₈O₅ requires: 656.3, found: m/z=657.3 [M+H]⁺.

### Example 305. (4aS,5aR)-5,5-difluoro-5a-methyl-N-{1-[(1rs,4rs)-4-(4-{5-[(3RS)-2,6-dioxopiperidin-3-yl]pyridin-2-yl}piperazine-1-carbonyl)cyclohexyl]-1H-pyrazol-4-yl}-1H,4H,4aH,5H,5aH,6H-cyclopropa[f]indazole-3-carboxamide (178)

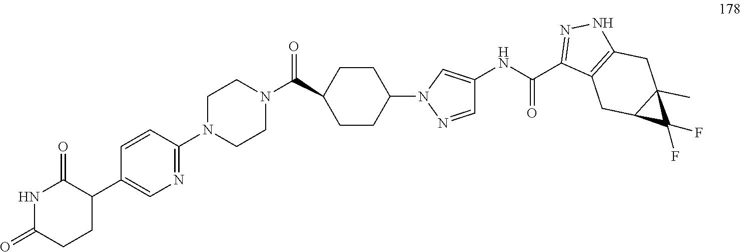

This compound was synthesized following the standard amide coupling conditions and procedure 19. LCMS: C₃₄H₃₉F₂N₉O₄ requires: 675.3, found: m/z=676.4 [M+H]⁺.

### Example 306. (4aS,5aR)-5,5-difluoro-5a-methyl-N-{1-[(1rs,4rs)-4-(4-{4-[(3RS)-2,6-dioxopiperidin-3-yl]phenyl}piperazine-1-carbonyl)cyclohexyl]-1H-pyrazol-4-yl}-1H,4H,4aH,5H,5aH,6H-cyclopropa[f]indazole-3-carboxamide (179)

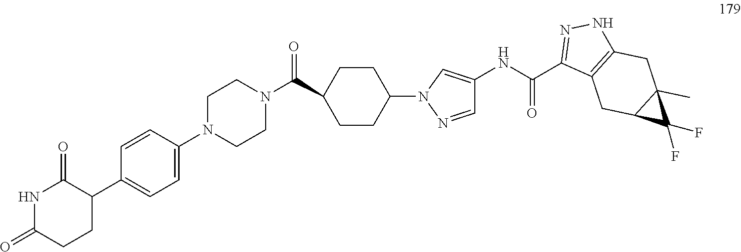

This compound was synthesized following the standard amide coupling conditions and procedure 19. LCMS: C₃₅H₄₀F₂N₈O₄ requires: 674.3, found: m/z=675.2 [M+H]⁺.

### Example 307. 5-[4-(4-{4-[(4aS,5aR)-5,5-difluoro-5a-methyl-1H,4H,4aH,5H,5aH,6H-cyclopropa[f]indazole-3-amido]-1H-pyrazol-1-yl}piperidine-1-carbonyl)piperidin-1-yl]-N-[(3RS)-2,6-dioxopiperidin-3-yl]pyridine-2-carboxamide (180)

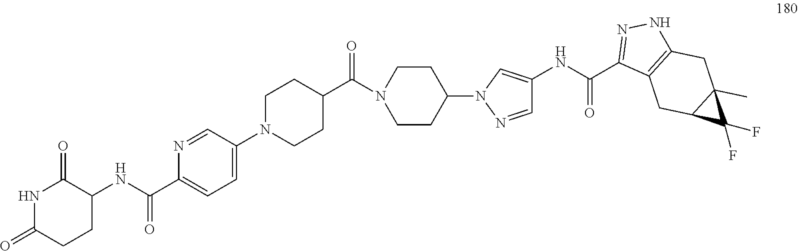 [SMILES: C[C@@]12Cc3[nH]nc(C(=O)Nc4cnn(C5CCN(C(=O)C6CCN(c7ccc(C(=O)NC8CCC(=O)NC8=O)nc7)CC6)CC5)c4)c3C[C@@H]1C2(F)F]

This compound was synthesized following the standard amide coupling conditions and procedure 19. LCMS: C₃₅H₄₀F₂N₁₀O₅ requires: 718.3, found: m/z=719.2 [M+H]⁺.

### Example 308. 3-(6-(4-((4-(2-(6,6-Dimethyl-4,5,6,7-tetrahydro-1H-indazol-3-yl)-1H-indole-6-carbonyl)piperazin-1-yl)methyl)piperidin-1-yl)-2-methylpyridin-3-yl)piperidine-2,6-dione (181)

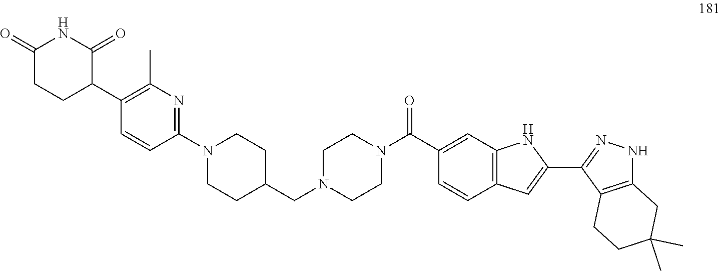 [SMILES: Cc1nc(N2CCC(CN3CCN(C(=O)c4ccc5cc(-c6n[nH]c7c6CCC(C)(C)C7)[nH]c5c4)CC3)CC2)ccc1C1CCC(=O)NC1=O]

### Step 1: Synthesis of 1-(5-(2,6-dioxopiperidin-3-yl)-6-methylpyridin-2-yl)piperidine-4-carbaldehyde

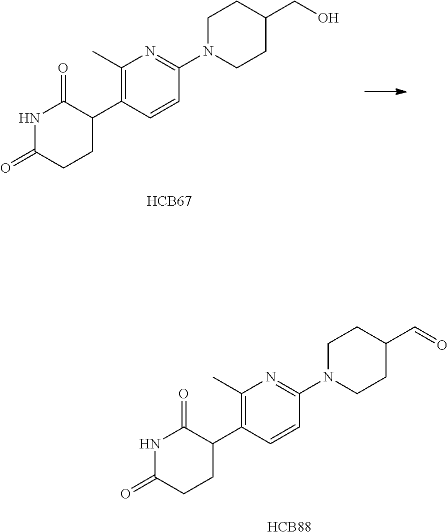 [SMILES: "HCB67" ; Cc1nc(N2CCC(CO)CC2)ccc1C1CCC(=O)NC1=O ; "HCB88" ; Cc1nc(N2CCC(C=O)CC2)ccc1C1CCC(=O)NC1=O]

To a mixture of 3-(6-(4-(hydroxymethyl)piperidin-1-yl)-2-methylpyridin-3-yl)piperidine-2,6-dione (HCB67)(19 mg, 0.06 mmol) in DMSO (1.00 mL) was added triethylamine (0.17 mL, 0.12 g, 1.20 mmol) followed by sulfur trioxide pyridine complex (96 mg, 0.60 mmol). After 30 minutes water was added. The mixture was extracted three times with DCM and concentrated to provide 1-(5-(2,6-dioxopiperidin-3-yl)-6-methylpyridin-2-yl)piperidine-4-carbaldehyde (HCB88) (19 mg, 100%). LCSM C₁₇H₂₁N₃O₃ requires: 315, found: m/z=316 [M+H]⁺.

### Step 2: 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methylpyridin-3-yl)piperidine-2,6-dione (HCB88)

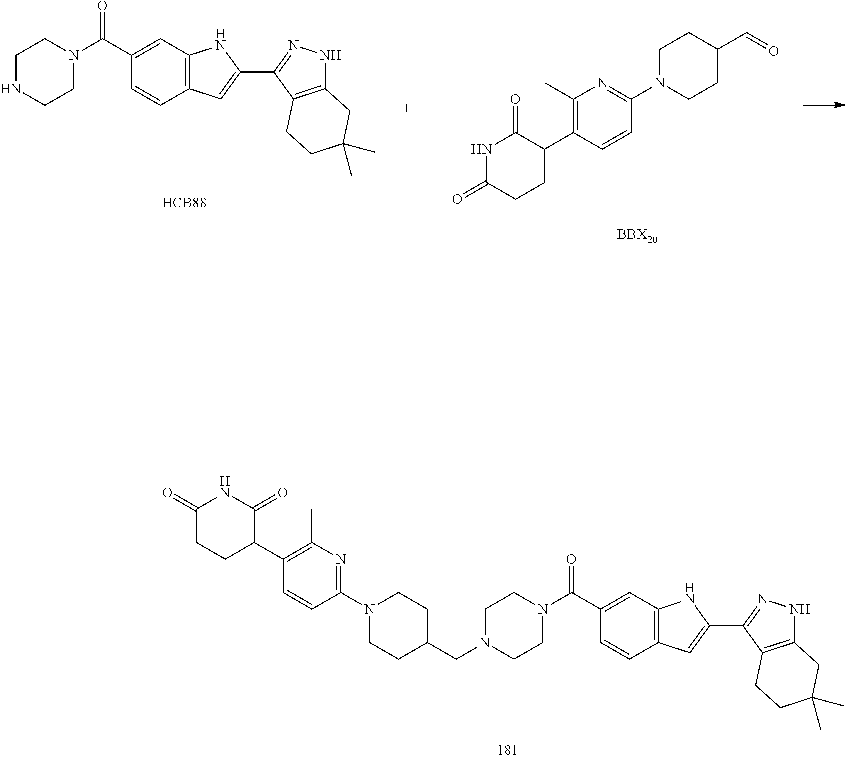 [SMILES: "HCB88" ; CC1(C)CCc2c(-c3cc4ccc(C(=O)N5CCNCC5)cc4[nH]3)n[nH]c2C1 ; Cc1nc(N2CCC(C=O)CC2)ccc1C1CCC(=O)NC1=O ; Cc1nc(N2CCC(CN3CCN(C(=O)c4ccc5cc(-c6n[nH]c7c6CCC(C)(C)C7)[nH]c5c4)CC3)CC2)ccc1C1CCC(=O)NC1=O]

1-(5-(2,6-dioxopiperidin-3-yl)-6-methylpyridin-2-yl)piperidine-4-carbaldehyde (19 mg, 0.06 mmol) was added to a mixture of (2-(6,6-dimethyl-4,5,6,7-tetrahydro-1H-indazol-3-yl)-1H-indol-6-yl)(piperazin-1-yl)methanone (BBX₂₀) (23 mg, 0.06 mmol) and N,N-diisopropylethylamine (0.08 mL, 0.06 g, 0.48 mmol) in DCM (2.00 mL) followed by sodium triacetoxyborohydride (38 mg, 0.18 mmol). After 1 hour, water was added. The mixture was extracted three times with DCM. The combined organic layers were concentrated. The crude residue was purified by preparative TLC eluted with 10% MeOH/DCM to provide 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methylpyridin-3-yl)piperidine-2,6-dione (0.0192 g, 46.3%). ¹H NMR (500 MHz, DMSO) δ 12.58 (s, 1H), 11.46 (s, 1H), 10.79 (s, 1H), 7.54 (d, J=8.1 Hz, 1H), 7.46 (s, 1H), 7.23 (d, J=8.7 Hz, 1H), 7.00 (d, J=8.1 Hz, 1H), 6.64-6.57 (m, 2H), 4.27 (d, J=12.4 Hz, 2H), 3.91 (dd, J=12.3, 4.9 Hz, 1H), 3.56-3.53 (m, 4H), 2.79-2.67 (m, 5H), 2.46-2.34 (m, 7H), 2.29 (s, 3H), 2.20 (d, J=6.7 Hz, 2H), 2.19-2.09 (m, 1H), 1.96-1.89 (m, 1H), 1.81-1.75 (m, 3H), 1.59 (t, J=6.4 Hz, 2H), 1.12-1.06 (m, 2H), 1.02 (s, 6H). LCSM C₃₉H₄₈N₈O₃ requires: 676, found: m/z=677 [M+H]⁺.

### Example 309. 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methylphenyl)piperidine-2,6-dione (182)

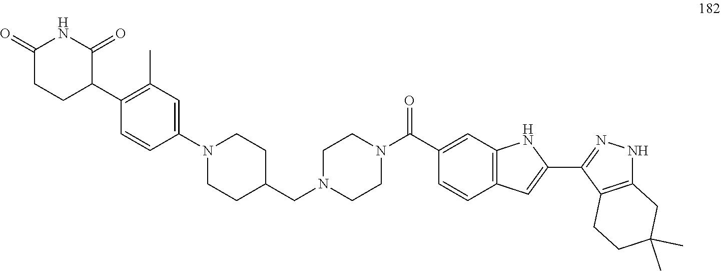 [SMILES: Cc1cc(N2CCC(CN3CCN(C(=O)c4ccc5cc(-c6n[nH]c7c6CCC(C)(C)C7)[nH]c5c4)CC3)CC2)ccc1C1CCC(=O)NC1=O]

### Step 1: Synthesis of 1-(4-(2,6-dioxopiperidin-3-yl)-3-methylphenyl)piperidine-4-carbaldehyde

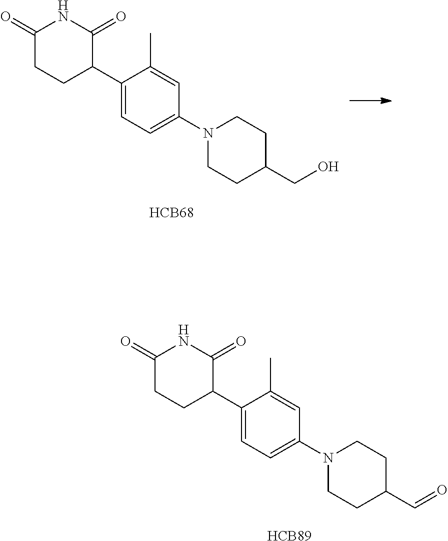 [SMILES: "HCB68" ; Cc1cc(N2CCC(CO)CC2)ccc1C1CCC(=O)NC1=O ; "HCB89" ; Cc1cc(N2CCC(C=O)CC2)ccc1C1CCC(=O)NC1=O]

To a mixture of 3-(4-(4-(hydroxymethyl)piperidin-1-yl)-2-methylphenyl)piperidine-2,6-dione (HCB68) (29 mg, 0.09 mmol) and triethylamine (0.25 mL, 0.19 g, 1.83 mmol) in DMSO (1.00 mL) was added sulfur trioxide pyridine complex (146 mg, 0.92 mmol). After 15 minutes, water was added. The mixture was extracted three times with DCM. The combined organic layers were concentrated to provide 1-(4-(2,6-dioxopiperidin-3-yl)-3-methylphenyl)piperidine-4-carbaldehyde (29 mg, 100%). LCSM C₁₈H₂₂N₂O₃ requires: 314, found: m/z=315 [M+H]⁺.

### Step 2: Synthesis of 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methylphenyl)piperidine-2,6-dione

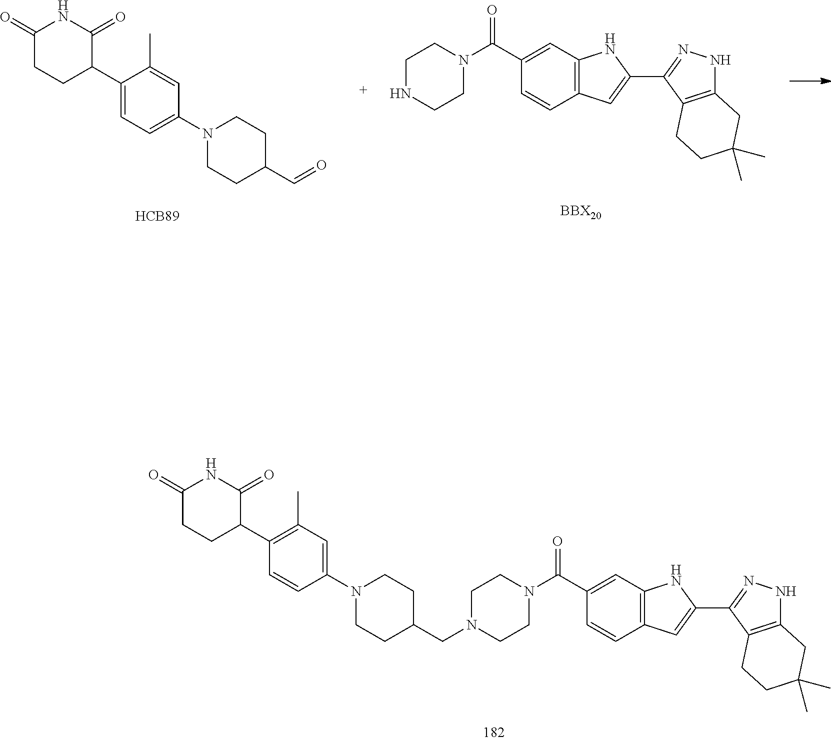 [SMILES: "HCB89" ; Cc1cc(N2CCC(C=O)CC2)ccc1C1CCC(=O)NC1=O ; CC1(C)CCc2c(-c3cc4ccc(C(=O)N5CCNCC5)cc4[nH]3)n[nH]c2C1 ; Cc1cc(N2CCC(CN3CCN(C(=O)c4ccc5cc(-c6n[nH]c7c6CCC(C)(C)C7)[nH]c5c4)CC3)CC2)ccc1C1CCC(=O)NC1=O]

To (2-(6,6-dimethyl-4,5,6,7-tetrahydro-1H-indazol-3-yl)-1H-indol-6-yl)(piperazin-1-yl)methanone (BBX₂₀) (34 mg, 0.09 mmol) in DCM (2.00 mL) was added N,N-diisopropylethylamine (HCB89) (0.13 mL, 0.09 g, 0.72 mmol) followed by 1-(4-(2,6-dioxopiperidin-3-yl)-3-methylphenyl)piperidine-4-carbaldehyde (29 mg, 0.09 mmol) in 1 mL DCM and finally sodium triacetoxyborohydride (57 mg, 0.27 mmol). After stirring for 1 hour, water was added. The mixture was extracted 3× DCM. The combined organic layers were concentrated. The crude residue was purified by preparative TLC eluted with 10% MeOH/DCM to provide 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methylphenyl)piperidine-2,6-dione (0.0312 g, 50.3%). ¹H NMR (500 MHz, DMSO) δ 12.59 (s, 1H), 11.46 (s, 1H), 10.77 (s, 1H), 7.54 (d, J=8.1 Hz, 1H), 7.46 (s, 1H), 7.01 (d, J=8.1 Hz, 1H), 6.90 (d, J=8.4 Hz, 1H), 6.77-6.69 (m, 2H), 6.62 (s, 1H), 3.90 (dd, J=12.3, 4.9 Hz, 1H), 3.66 (d, J=11.8 Hz, 2H), 3.60-3.47 (m, 4H), 2.75-2.58 (m, 5H), 2.45-2.34 (m, 7H), 2.24-2.19 (m, 5H), 2.17-2.06 (m, 1H), 1.99-1.92 (m, 1H), 1.80 (d, J=12.7 Hz, 2H), 1.68-1.65 (m, 1H), 1.62-1.56 (m, 2H), 1.27-1.18 (m, 2H), 1.02 (s, 6H). LCSM C₄₀H₄₉N₇O₃ requires: 675, found: m/z=676 [M+H]⁺.

### Example 310. 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)-5-fluoropyridin-3-yl)piperidine-2,6-dione (183)

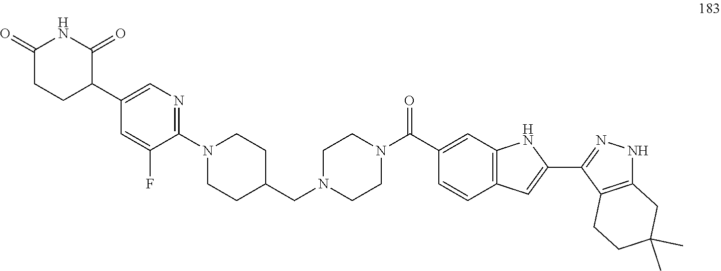 [SMILES: CC1(C)CCc2c(-c3cc4ccc(C(=O)N5CCN(CC6CCN(c7ncc(C8CCC(=O)NC8=O)cc7F)CC6)CC5)cc4[nH]3)n[nH]c2C1]

### Step 1: Synthesis of 1-(5-(2,6-dioxopiperidin-3-yl)-3-fluoropyridin-2-yl)piperidine-4-carbaldehyde

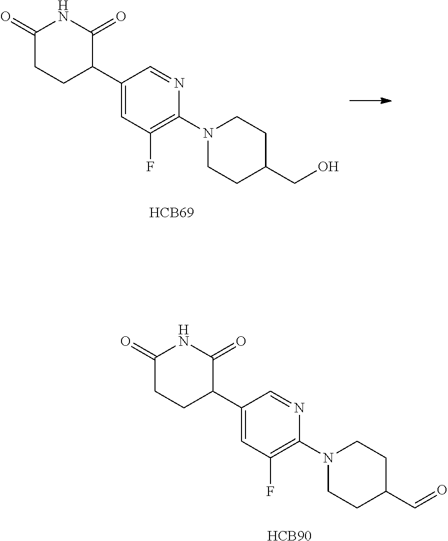 [SMILES: "HCB69" ; O=C1CCC(c2cnc(N3CCC(CO)CC3)c(F)c2)C(=O)N1 ; "HCB90" ; O=CC1CCN(c2ncc(C3CCC(=O)NC3=O)cc2F)CC1]

To a mixture of 3-(5-fluoro-6-(4-(hydroxymethyl)piperidin-1-yl)pyridin-3-yl)piperidine-2,6-dione (HCB69) (33 mg, 0.10 mmol) in DMSO (1.00 mL) was added triethylamine (0.29 mL, 0.21 g, 2.05 mmol) followed by sulfur trioxide pyridine complex (163 mg, 1.03 mmol). After 20 minutes, water was added and the mixture was extracted twice with DCM. The combined organic layers were concentrated to provide rac-1-{5-[(3R)-2,6-dioxopiperidin-3-yl]-3-fluoropyridin-2-yl}piperidine-4-carbaldehyde (33 mg, 100%). LCSM C₁₆H₁₈FN₃O₃ requires: 319, found: m/z=320 [M+H]⁺.

### Step 2: Synthesis of 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)-5-fluoropyridin-3-yl)piperidine-2,6-dione

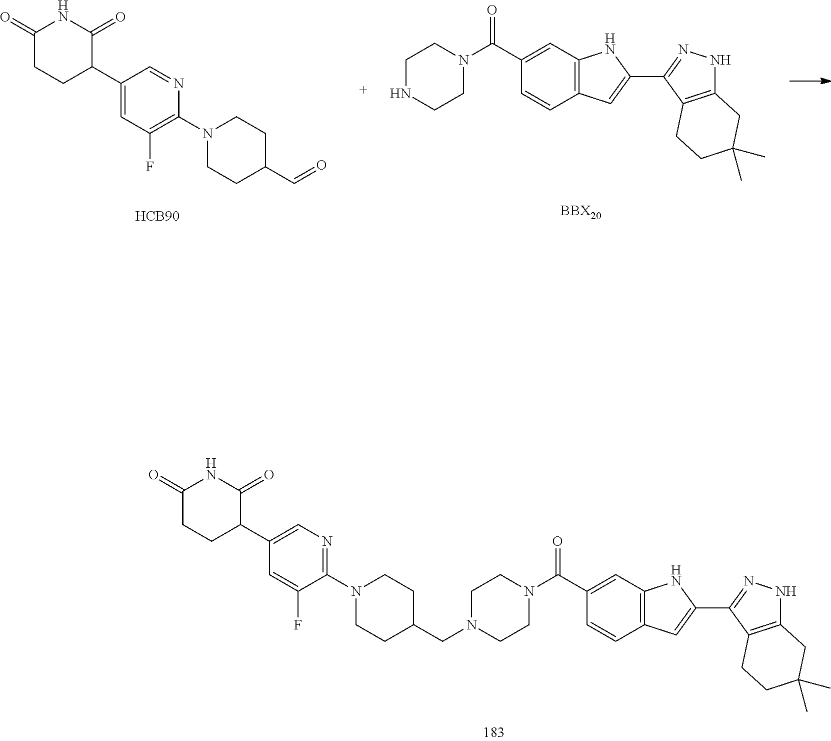 [SMILES: "HCB90" ; O=CC1CCN(c2ncc(C3CCC(=O)NC3=O)cc2F)CC1 ; CC1(C)CCc2c(-c3cc4ccc(C(=O)N5CCNCC5)cc4[nH]3)n[nH]c2C1 ; CC1(C)CCc2c(-c3cc4ccc(C(=O)N5CCN(CC6CCN(c7ncc(C8CCC(=O)NC8=O)cc7F)CC6)CC5)cc4[nH]3)n[nH]c2C1]

To a mixture of (2-(6,6-dimethyl-4,5,6,7-tetrahydro-1H-indazol-3-yl)-1H-indol-6-yl)(piperazin-1-yl)methanone (BBX₂₀) (38 mg, 0.10 mmol) in DCM (2.00 mL) was added N,N-diisopropylethylamine (0.14 mL, 0.10 g, 0.80 mmol). The mixture was added to 1-(5-(2,6-dioxopiperidin-3-yl)-3-fluoropyridin-2-yl)piperidine-4-carbaldehyde (HCB90) (32 mg, 0.10 mmol) followed by sodium triacetoxyborohydride (64 mg, 0.30 mmol). After stirring overnight, the mixture was diluted with water and extracted twice with DCM. The combined organic layers were concentrated. The crude residue was purified by preparative TLC eluted with 10% MeOH/DCM to provide 3-(6-(4-((4-(2-(6,6-dimethyl-4,5,6,7-tetrahydro-1H-indazol-3-yl)-1H-indole-6-carbonyl)piperazin-1-yl)methyl)piperidin-1-yl)-5-fluoropyridin-3-yl)piperidine-2,6-dione (183) (0.0451 g, 64.9%). ¹H NMR (500 MHz, DMSO) δ 12.58 (s, 1H), 11.46 (s, 1H), 10.86 (s, 1H), 7.87 (s, 1H), 7.54 (d, J=8.1 Hz, 1H), 7.48-7.40 (m, 2H), 7.01 (d, J=8.2 Hz, 1H), 6.62 (s, 1H), 3.94 (d, J=12.5 Hz, 2H), 3.85 (dd, J=11.9, 4.2 Hz, 1H), 3.64-3.48 (m, 4H), 2.83 (t, J=12.3 Hz, 2H), 2.75-2.62 (m, 4H), 2.42 (d, J=13.8 Hz, 6H), 2.31-2.19 (m, 3H), 2.02-1.99 (m, 1H), 1.85-1.72 (m, 3H), 1.59 (t, J=6.6 Hz, 2H), 1.27-1.19 (m, 2H), 1.02 (s, 6H). LCSM C₃₈H₄₅FN₈O₃ requires: 680, found: m/z=681 [M+H]⁺.

### Example 311. 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methoxyphenyl)piperidine-2,6-dione (184)

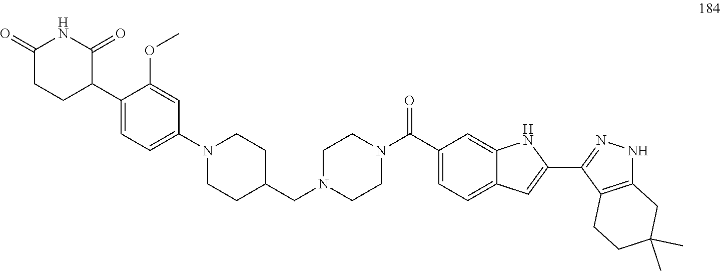 [SMILES: COc1cc(N2CCC(CN3CCN(C(=O)c4ccc5cc(-c6n[nH]c7c6CCC(C)(C)C7)[nH]c5c4)CC3)CC2)ccc1C1CCC(=O)NC1=O]

### Step 1: Synthesis of 1-(4-(2,6-dioxopiperidin-3-yl)-3-methoxyphenyl)piperidine-4-carbaldehyde

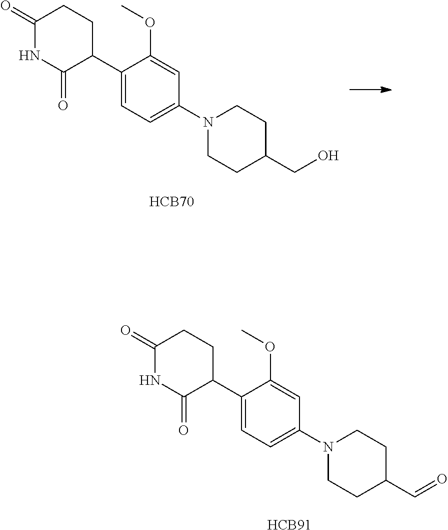 [SMILES: "HCB70" ; COc1cc(N2CCC(CO)CC2)ccc1C1CCC(=O)NC1=O ; "HCB91" ; COc1cc(N2CCC(C=O)CC2)ccc1C1CCC(=O)NC1=O]

Sulfur trioxide pyridine complex (158 mg, 0.99 mmol) was added to a mixture of 3-(4-(4-(hydroxymethyl)piperidin-1-yl)-2-methoxyphenyl)piperidine-2,6-dione (33 mg, 0.10 mmol) and triethylamine (0.28 mL, 0.20 g, 1.99 mmol) in DMSO (1.00 mL). After 25 minutes, water was added and the mixture was extracted 3× DCM. The combined organic layers were concentrated to provide 1-(4-(2,6-dioxopiperidin-3-yl)-3-methoxyphenyl)piperidine-4-carbaldehyde (33 mg, 100%). LCSM C₁₈H₂₂N₂O₄ requires: 330, found: m/z=331 [M+H]⁺.

### Step 2: 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methoxyphenyl)piperidine-2,6-dione

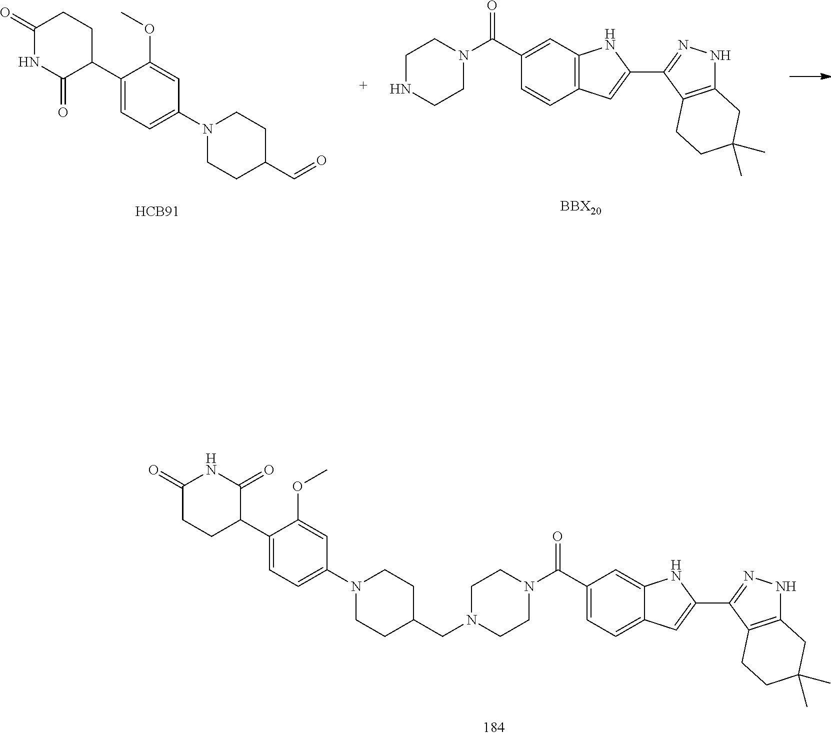 [SMILES: "HCB91" ; COc1cc(N2CCC(C=O)CC2)ccc1C1CCC(=O)NC1=O ; CC1(C)CCc2c(-c3cc4ccc(C(=O)N5CCNCC5)cc4[nH]3)n[nH]c2C1 ; COc1cc(N2CCC(CN3CCN(C(=O)c4ccc5cc(-c6n[nH]c7c6CCC(C)(C)C7)[nH]c5c4)CC3)CC2)ccc1C1CCC(=O)NC1=O]

N,N-diisopropylethylamine (0.14 mL, 0.10 g, 0.80 mmol) was added to a mixture of (2-(6,6-dimethyl-4,5,6,7-tetrahydro-1H-indazol-3-yl)-1H-indol-6-yl)(piperazin-1-yl)methanone (BBX₂₀) (38 mg, 0.10 mmol) in DCM (2.00 mL). 1-(4-(2,6-dioxopiperidin-3-yl)-3-methoxyphenyl)piperidine-4-carbaldehyde (HCB91) (33 mg, 0.10 mmol) in 1 mL DCM was added. After 90 minutes, water was added. The mixture was extracted twice with 10% MeOH/DCM. The combined organic layers were concentrated. The crude residue was purified by preparative TLC eluted with 10% MeOH/DCM to provide 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-methoxyphenyl)piperidine-2,6-dione (0.0305 g, 43.2%). ¹H NMR (500 MHz, DMSO) δ 12.58 (s, 1H), 11.46 (s, 1H), 10.67 (s, 1H), 7.54 (d, J=8.0 Hz, 1H), 7.46 (s, 1H), 7.01 (d, J=8.1 Hz, 1H), 6.91 (d, J=8.3 Hz, 1H), 6.62 (s, 1H), 6.54 (d, J=2.3 Hz, 1H), 6.45 (dd, J=8.5, 2.3 Hz, 1H), 3.78 (dd, J=11.4, 5.1 Hz, 1H), 3.74-3.66 (m, 5H), 3.63-3.44 (m, 4H), 2.75-2.58 (m, 5H), 2.49-2.31 (m, 7H), 2.22 (d, J=7.1 Hz, 2H), 2.19-2.07 (m, 1H), 1.91-1.83 (m, 1H), 1.81 (d, J=12.7 Hz, 2H), 1.75-1.63 (m, 1H), 1.59 (t, J=6.4 Hz, 2H), 1.26-1.17 (m, 2H), 1.02 (s, 6H). LCSM C₄₀H₄₉N₇O₄ requires: 691, found: m/z=692 [M+H]⁺.

### Example 312. 3-(2-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)pyridin-4-yl)piperidine-2,6-dione (185)

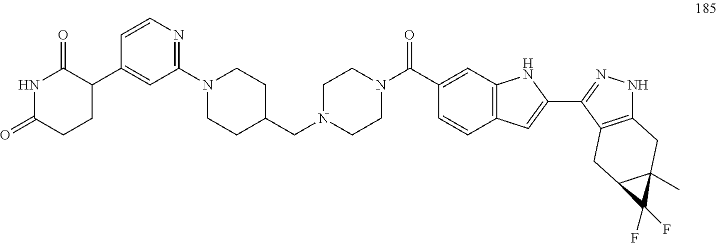 [SMILES: C[C@@]12Cc3[nH]nc(-c4cc5ccc(C(=O)N6CCN(CC7CCN(c8cc(C9CCC(=O)NC9=O)ccn8)CC7)CC6)cc5[nH]4)c3C[C@@H]1C2(F)F]

### Step 1: Synthesis of 1-(4-(2,6-dioxopiperidin-3-yl)pyridin-2-yl)piperidine-4-carbaldehyde

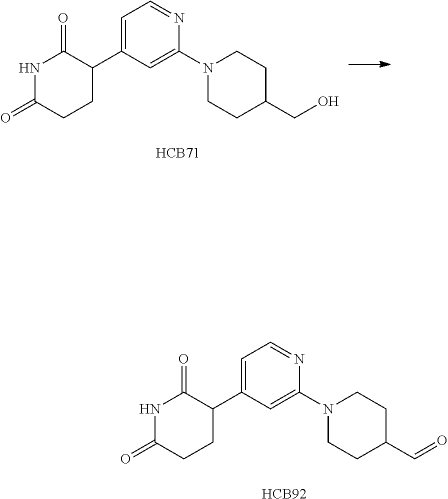 [SMILES: "HCB71" ; O=C1CCC(c2ccnc(N3CCC(CO)CC3)c2)C(=O)N1 ; "HCB92" ; O=CC1CCN(c2cc(C3CCC(=O)NC3=O)ccn2)CC1]

To a mixture of 3-(2-(4-(hydroxymethyl)piperidin-1-yl)pyridin-4-yl)piperidine-2,6-dione (HCB71) (12 mg, 0.04 mmol) in DMSO (1.00 mL) was added triethylamine (0.11 mL, 0.08 g, 0.79 mmol) followed by sulfur trioxide pyridine complex (63 mg, 0.40 mmol). After 15 minutes water was added. The mixture was extracted three times with DCM. The combined organic layers were concentrated to provide 1-(4-(2,6-dioxopiperidin-3-yl)pyridin-2-yl)piperidine-4-carbaldehyde (12 mg, 100%). LCSM C₁₆H₁₉N₃O₃ requires: 301, found: m/z=302 [M+H]⁺.

### Step 2: Synthesis of 3-(2-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)pyridin-4-yl)piperidine-2,6-dione

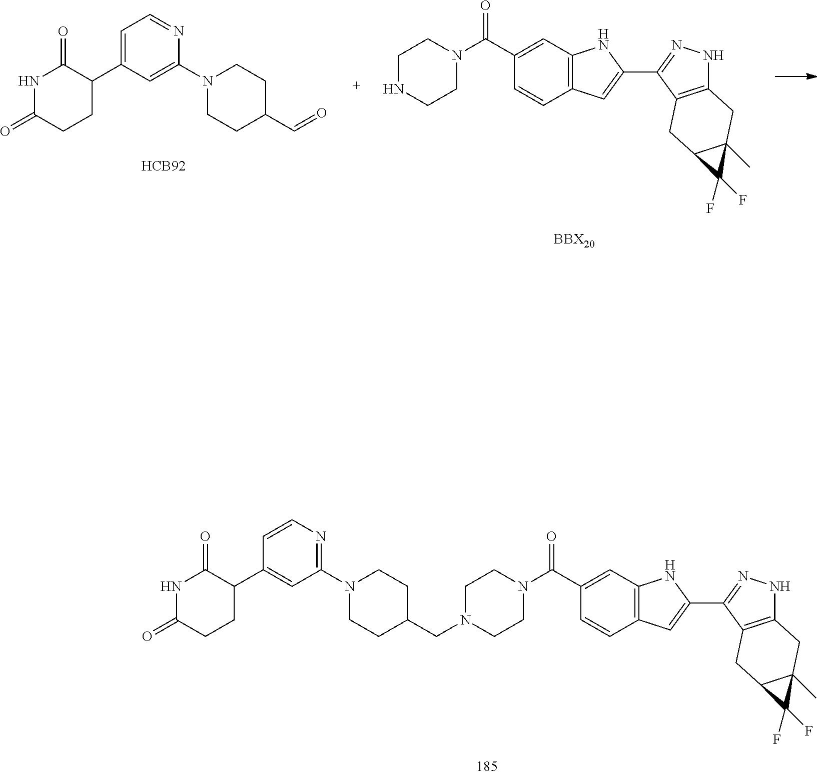 [SMILES: "HCB92" ; O=CC1CCN(c2cc(C3CCC(=O)NC3=O)ccn2)CC1 ; C[C@@]12Cc3[nH]nc(-c4cc5ccc(C(=O)N6CCNCC6)cc5[nH]4)c3C[C@@H]1C2(F)F ; C[C@@]12Cc3[nH]nc(-c4cc5ccc(C(=O)N6CCN(CC7CCN(c8cc(C9CCC(=O)NC9=O)ccn8)CC7)CC6)cc5[nH]4)c3C[C@@H]1C2(F)F]

To a mixture of (2-((4aS,5aR)-5,5-difluoro-5a-methyl-1,4,4a,5,5a,6-hexahydrocyclopropa[f]indazol-3-yl)-1H-indol-6-yl)(piperazin-1-yl)methanone (165 mg, 0.04 mmol) in DCM (1.00 mL) was added N,N-diisopropylethylamine (0.06 mL, 0.04 g, 0.32 mmol). The resulting mixture was added to 1-(4-(2,6-dioxopiperidin-3-yl)pyridin-2-yl)piperidine-4-carbaldehyde (12 mg, 0.04 mmol) followed by sodium triacetoxyborohydride (25 mg, 0.12 mmol). After 1 hour, water was added and the mixture was extracted three times with DCM. The combined organic layers were concentrated. The crude residue was purified by preparative TLC eluted with 10% MeOH/DCM to provide 3-(2-(4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)pyridin-4-yl)piperidine-2,6-dione (0.0097 g, 34.5%). ¹H NMR (500 MHz, DMSO) δ 12.71 (s, 1H), 11.49 (s, 1H), 10.86 (s, 1H), 8.02 (d, J=5.1 Hz, 1H), 7.55 (d, J=8.1 Hz, 1H), 7.46 (s, 1H), 7.02 (d, J=7.6 Hz, 1H), 6.69 (s, 1H), 6.64 (s, 1H), 6.46 (d, J=5.1 Hz, 1H), 4.28 (d, J=12.6 Hz, 2H), 3.76 (dd, J=11.8, 5.0 Hz, 1H), 3.63-3.48 (m, 4H), 3.18-2.97 (m, 2H), 2.90-2.73 (m, 3H), 2.65 (ddd, J=17.1, 12.1, 5.3 Hz, 1H), 2.55 (s, 1H), 2.42-2.38 (m, 4H), 2.30-2.16 (m, 3H), 2.04-1.96 (m, 1H), 1.92-1.74 (m, 3H), 1.40 (s, 3H), 1.30-1.23 (m, 2H), 1.13-1.07 (m, 2H). LCSM C₃₈H₄₂F₂N₈O₃ requires: 696, found: m/z=697 [M+H]⁺.

### Example 313. 3-(2-(4-(2-(4-(2-(6,6-dimethyl-4,5,6,7-tetrahydro-1H-indazol-3-yl)-1H-indole-6-carbonyl)piperazin-1-yl)ethyl)piperidin-1-yl)pyridin-4-yl)piperidine-2,6-dione (186)

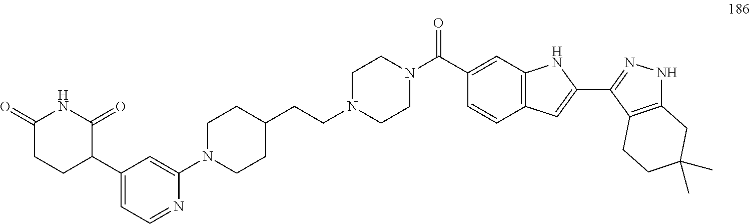 [SMILES: CC1(C)CCc2c(-c3cc4ccc(C(=O)N5CCN(CCC6CCN(c7cc(C8CCC(=O)NC8=O)ccn7)CC6)CC5)cc4[nH]3)n[nH]c2C1]

### Step 1: Synthesis of 2-(1-(4-(2,6-dioxopiperidin-3-yl)pyridin-2-yl)piperidin-4-yl)acetaldehyde

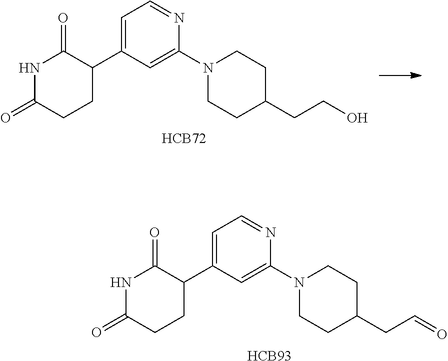

Sulfur trioxide pyridine complex (105 mg, 0.66 mmol) was added to a mixture of 3-(2-(4-(2-hydroxyethyl)piperidin-1-yl)pyridin-4-yl)piperidine-2,6-dione (21 mg, 0.07 mmol) and triethylamine (0.18 mL, 0.13 g, 1.32 mmol) in DMSO (1.00 mL). After 20 minutes, the mixture was diluted with water and extracted three times with DCM. The combined organic layers were concentrated and pumped on to provide 2-(1-(4-(2,6-dioxopiperidin-3-yl)pyridin-2-yl)piperidin-4-yl)acetaldehyde (21 mg, 100%). LCSM C₁₇H₂₁N₃O₃ requires: 315, found: m/z=316 [M+H]⁺.

### Step 2: Synthesis of 3-(2-(4-(2-(4-(2-(6,6-dimethyl-4,5,6,7-tetrahydro-1H-indazol-3-yl)-1H-indole-6-carbonyl)piperazin-1-yl)ethyl)piperidin-1-yl)pyridin-4-yl)piperidine-2,6-dione

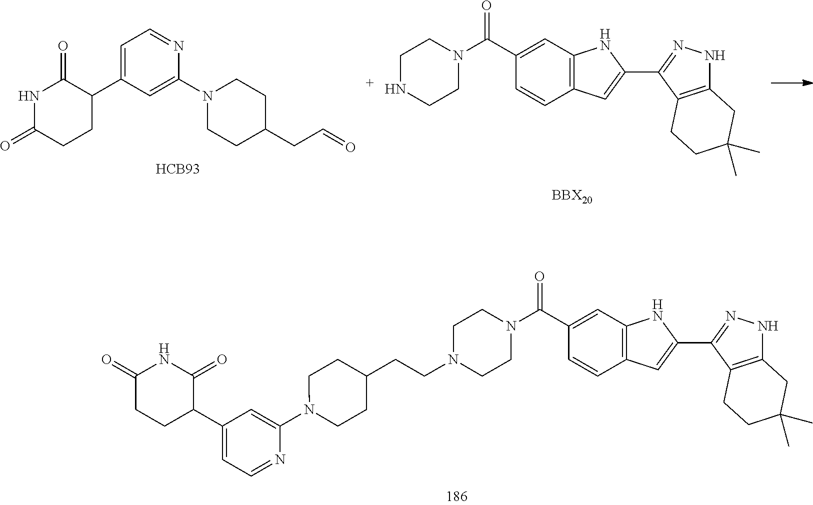

2-(1-(4-(2,6-dioxopiperidin-3-yl)pyridin-2-yl)piperidin-4-yl)acetaldehyde (HCB93) (21 mg, 0.07 mmol) in DCM (1.00 mL) was added to a mixture of 6,6-dimethyl-3-[6-(piperazine-1-carbonyl)-1H-indol-2-yl]-1,4,5,7-tetrahydroindazole (BBX₂₀) (26.4 mg, 0.07 mmol) and N,N-diisopropylethylamine (0.10 mL, 0.07 g, 0.56 mmol) in DCM (1.00 mL). The mixture was sonicated. After 5 minutes, sodium triacetoxyborohydride (45 mg, 0.21 mmol) was added. After 2 hours, water was added and the mixture was extracted twice with DCM. The combined organic layers were concentrated. The crude residue was purified by preparative TLC eluted with 10% MeOH/DCM to provide 3-(2-(4-(2-(4-(2-(6,6-dimethyl-4,5,6,7-tetrahydro-1H-indazol-3-yl)-1H-indole-6-carbonyl)piperazin-1-yl)ethyl)piperidin-1-yl)pyridin-4-yl)piperidine-2,6-dione (0.0195 g, 40.3%). ¹H NMR (500 MHz, DMSO) δ 12.57 (s, 1H), 11.45 (s, 1H), 10.86 (s, 1H), 8.02 (d, J=5.1 Hz, 1H), 7.54 (d, J=8.1 Hz, 1H), 7.45 (s, 1H), 7.00 (d, J=8.1 Hz, 1H), 6.69 (s, 1H), 6.62 (s, 1H), 6.46 (d, J=5.2 Hz, 1H), 4.27 (d, J=12.8 Hz, 2H), 3.76 (dd, J=11.8, 4.9 Hz, 1H), 3.55-3.51 (m, 4H), 2.80-2.60 (m, 5H), 2.45-2.36 (m, 10H), 2.30-2.18 (m, 1H), 2.05-1.96 (m, 1H), 1.73 (d, J=12.7 Hz, 2H), 1.62-1.54 (m, 2H), 1.45-1.39 (m, 2H), 1.18-1.07 (m, 2H), 1.02 (s, 6H). LCSM C₃₉H₄₈N₈O₃ requires: 676, found: m/z=677 [M+H]⁺.

### Example 314. 3-(2-(4-((4-(2-(6,6-dimethyl-4,5,6,7-tetrahydro-1H-indazol-3-yl)-1H-indole-6-carbonyl)piperazin-1-yl)methyl)piperidin-1-yl)pyrimidin-5-yl)piperidine-2,6-dione (187)

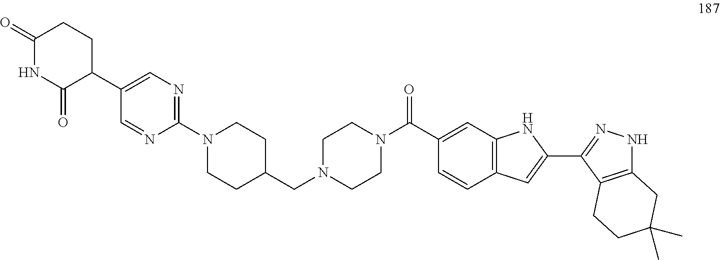 [SMILES: CC1(C)CCc2c(-c3cc4ccc(C(=O)N5CCN(CC6CCN(c7ncc(C8CCC(=O)NC8=O)cn7)CC6)CC5)cc4[nH]3)n[nH]c2C1]

### Step 1: Synthesis of 1-(5-(2,6-dioxopiperidin-3-yl)pyrimidin-2-yl)piperidine-4-carbaldehyde

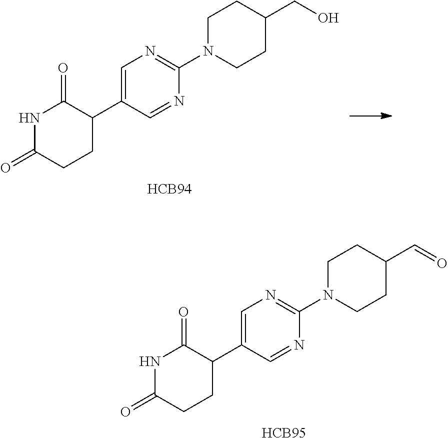

To a mixture of 3-(2-(4-(hydroxymethyl)piperidin-1-yl)pyrimidin-5-yl)piperidine-2,6-dione (HCB94) (24 mg, 0.08 mmol) and triethylamine (0.22 mL, 0.16 g, 1.58 mmol) in DMSO (1.00 mL) was added sulfur trioxide pyridine complex (126 mg, 0.79 mmol). After 15 minutes, water was added. The mixture was extracted three times with DCM. The combined organic layers were concentrated to provide 1-(5-(2,6-dioxopiperidin-3-yl)pyrimidin-2-yl)piperidine-4-carbaldehyde (24 mg, 100%). LCSM C₁₅H₁₈N₄O₃ requires: 302, found: m/z=303 [M+H]⁺.

### Step 2: Synthesis of 3-(2-(4-((4-(2-(6,6-dimethyl-4,5,6,7-tetrahydro-1H-indazol-3-yl)-1H-indole-6-carbonyl)piperazin-1-yl)methyl)piperidin-1-yl)pyrimidin-5-yl)piperidine-2,6-dione

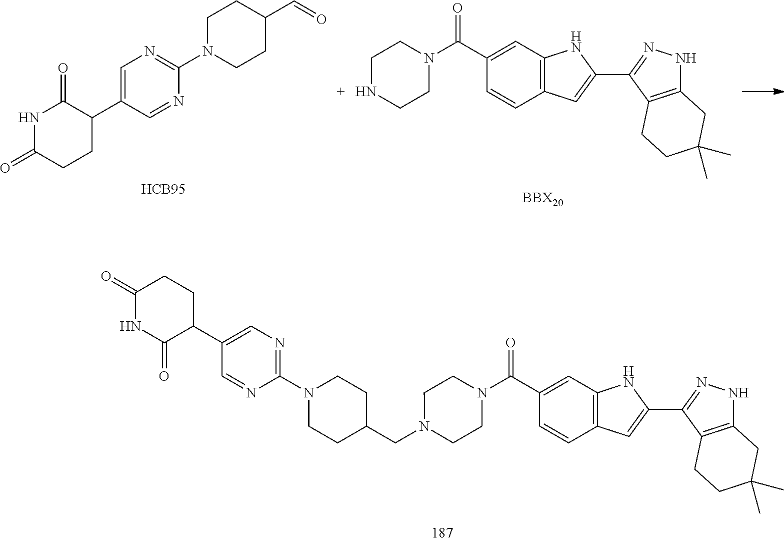

A mixture of 1-(5-(2,6-dioxopiperidin-3-yl)pyrimidin-2-yl)piperidine-4-carbaldehyde (24 mg, 0.08 mmol) in DCM (1.00 mL) was added to a mixture of 6,6-dimethyl-3-[6-(piperazine-1-carbonyl)-1H-indol-2-yl]-1,4,5,7-tetrahydroindazole (BBX₂₀) (23 mg, 0.06 mmol) and N,N-diisopropylethylamine (0.11 mL, 0.08 g, 0.64 mmol) in DCM (1.00 mL). The mixture was sonicated for a couple minutes then sodium triacetoxyborohydride (51 mg, 0.24 mmol) was added. After 1 hour 15 minutes, water was added. The mixture was extracted twice with DCM. The combined organic layers were concentrated in vacuo. The crude residue was purified by preparative TLC eluted with 10% MeOH/DCM to provide 3-(2-(4-((4-(2-(6,6-dimethyl-4,5,6,7-tetrahydro-1H-indazol-3-yl)-1H-indole-6-carbonyl)piperazin-1-yl)methyl)piperidin-1-yl)pyrimidin-5-yl)piperidine-2,6-dione (0.0072 g, 13.3%). ¹H NMR (500 MHz, DMSO) δ 12.58 (s, 1H), 11.46 (s, 1H), 10.86 (s, 1H), 8.22 (s, 2H), 7.54 (d, J=8.1 Hz, 1H), 7.46 (s, 1H), 7.01 (d, J=8.0 Hz, 1H), 6.62 (s, 1H), 4.64 (d, J=12.8 Hz, 2H), 3.73 (dd, J=12.8, 4.9 Hz, 1H), 3.56-3.53 (m, 4H), 2.87 (t, J=12.4 Hz, 2H), 2.75-2.64 (m, 3H), 2.60-2.54 (m, 1H), 2.47-2.33 (m, 6H), 2.29-2.15 (m, 3H), 2.02-1.95 (m, 1H), 1.91-1.74 (m, 3H), 1.59 (t, J=6.2 Hz, 2H), 1.25 (s, 1H), 1.11-0.97 (m, 8H). LCSM C₃₇H₄₅N₉O₃ requires: 663, found: m/z=664 [M+H]⁺.

### Example 315. (2S)—N-(2-(6,6-dimethyl-4,5,6,7-tetrahydro-1H-indazol-3-yl)-5-fluoro-1H-indol-6-yl)-2-(4-((1-(5-(2,6-dioxopiperidin-3-yl)pyridin-2-yl)piperidin-4-yl)methyl)piperazin-1-yl)-N-methylpropanamide (188)

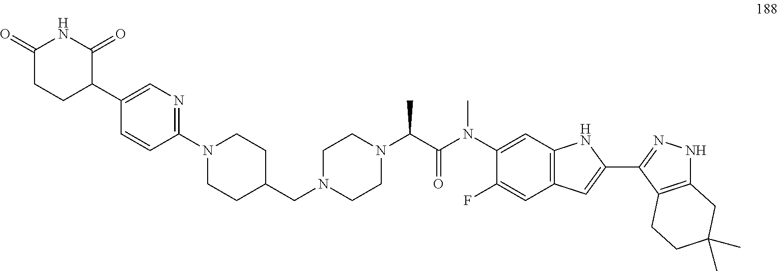 [SMILES: C[C@@H](C(=O)N(C)c1cc2[nH]c(-c3n[nH]c4c3CCC(C)(C)C4)cc2cc1F)N1CCN(CC2CCN(c3ccc(C4CCC(=O)NC4=O)cn3)CC2)CC1]

### Step 1: Synthesis of Synthesis of tert-butyl (2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)(methyl)carbamate

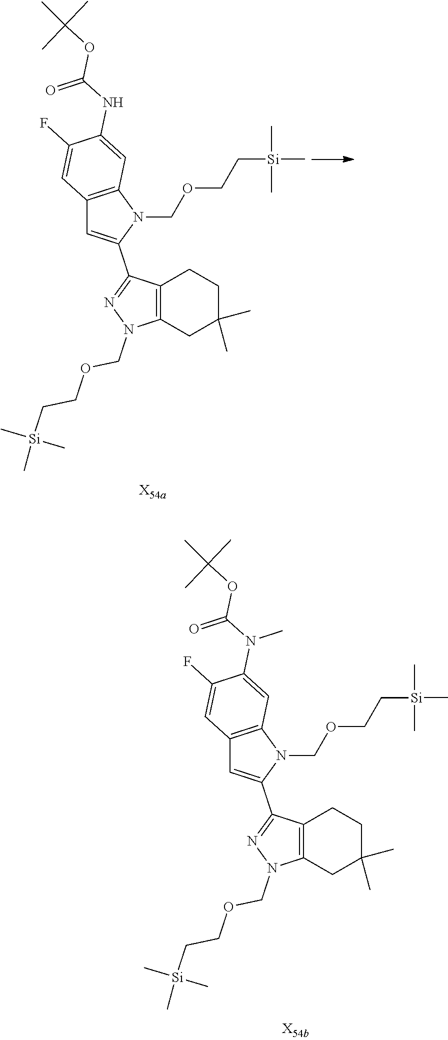

Sodium hydride 60% (15 mg, 0.36 mmol) was added to a 0° C. mixture of tert-butyl N-[2-(6,6-dimethyl-1-{[2-(trimethylsilyl)ethoxy]methyl}-5,7-dihydro-4H-indazol-3-yl)-5-fluoro-1-{[2-(trimethylsilyl)ethoxy]methyl}indol-6-yl]carbamate (200 mg, 0.30 mmol) in DMF (2.00 mL). After 25 minutes, methyl iodide (0.02 mL, 51.69 mg, 0.36 mmol) was added. After 1 hour, water was added and the mixture was extracted with ethylacetate. The organic layer was concentrated. The crude residue was purified by flash chromatography on a 24 g column eluted with 0 to 20% ethylacetate/hexanes to provide tert-butyl (2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)(methyl)carbamate (0.152 g, 74.4%). LCSM C₃₅H₅₇FN₄O₄Si₂ requires: 672, found: m/z=673 [M+H]⁺.

### Step 2: Synthesis of 2-(6,6-dimethyl-1-((2-(trimethylsilyl)ethoxy)methyl)-4,5,6,7-tetrahydro-1H-indazol-3-yl)-5-fluoro-N-methyl-1-((2-(trimethylsilyl)ethoxy)methyl)-1H-indol-6-amine

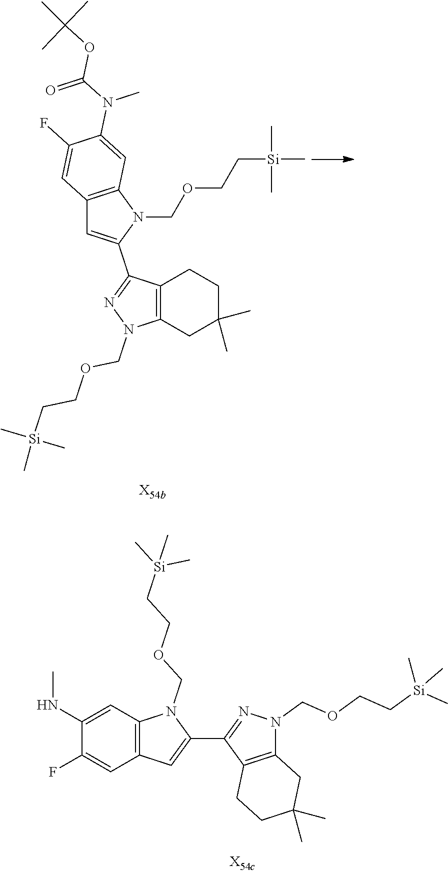

tert-Butyl (2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)(methyl)carbamate (126 mg, 0.19 mmol) was dissolved in DCM (1.00 mL) and TFA (1.00 mL). After 15 minutes, the mixture was concentrated to provide 2-(6,6-dimethyl-1-((2-(trimethylsilyl)ethoxy)methyl)-4,5,6,7-tetrahydro-1H-indazol-3-yl)-5-fluoro-N-methyl-1-((2-(trimethylsilyl)ethoxy)methyl)-1H-indol-6-amine (108 mg, 100%). LCSM C₃₀H₄₉FN₄O₂Si₂ requires: 572, found: m/z=573 [M+H]⁺.

### Step 3: Synthesis of tert-butyl (S)-4-(1-((2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)(methyl)amino)-1-oxopropan-2-yl)piperazine-1-carboxylate

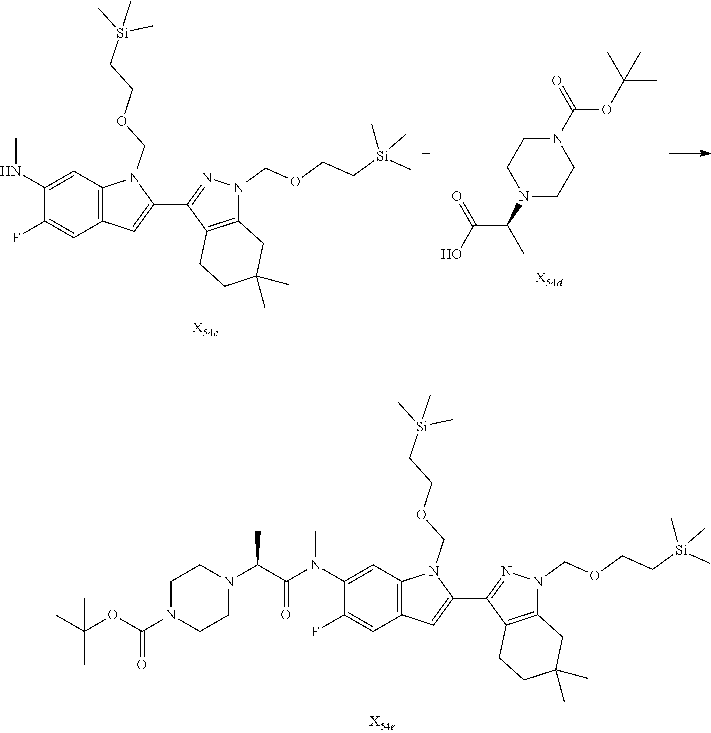 [SMILES: CNc1cc2c(cc1F)cc(-c1nn(COCC[Si](C)(C)C)c3c1CCC(C)(C)C3)n2COCC[Si](C)(C)C ; C[C@@H](C(=O)O)N1CCN(C(=O)OC(C)(C)C)CC1 ; C[C@@H](C(=O)N(C)c1cc2c(cc1F)cc(-c1nn(COCC[Si](C)(C)C)c3c1CCC(C)(C)C3)n2COCC[Si](C)(C)C)N1CCN(C(=O)OC(C)(C)C)CC1]

To a mixture of 2-(6,6-dimethyl-1-((2-(trimethylsilyl)ethoxy)methyl)-4,5,6,7-tetrahydro-1H-indazol-3-yl)-5-fluoro-N-methyl-1-((2-(trimethylsilyl)ethoxy)methyl)-1H-indol-6-amine (109 mg, 0.19 mmol) in DMF (1.00 mL) was added N,N-diisopropylethylamine (0.26 mL, 0.20 g, 1.52 mmol). (2S)-2-[4-(tert-butoxycarbonyl)piperazin-1-yl]propanoic acid (49 mg, 0.19 mmol) was added followed by HATU (72 mg, 0.19 mmol). After 2 hours, the mixture was heated to 60° C. for 18 hours. Water was added and the mixture was extracted with ethyl acetate. The organic layer was concentrated then purified by flash chromatography on a 24 g column eluted with 0 to 60% ethylacetate/hexanes to provide tert-butyl (S)-4-(1-((2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)(methyl)amino)-1-oxopropan-2-yl)piperazine-1-carboxylate (0.0319 g, 20.6%). LCSM C₄₂H₆₉FN₆O₅Si₂ requires: 812, found: m/z=813 [M+H]⁺.

### Step 4: Synthesis of tert-butyl (S)-4-(1-((2-(6,6-dimethyl-4,5,6,7-tetrahydro-1H-indazol-3-yl)-5-fluoro-1H-indol-6-yl)(methyl)amino)-1-oxopropan-2-yl)piperazine-1-carboxylate

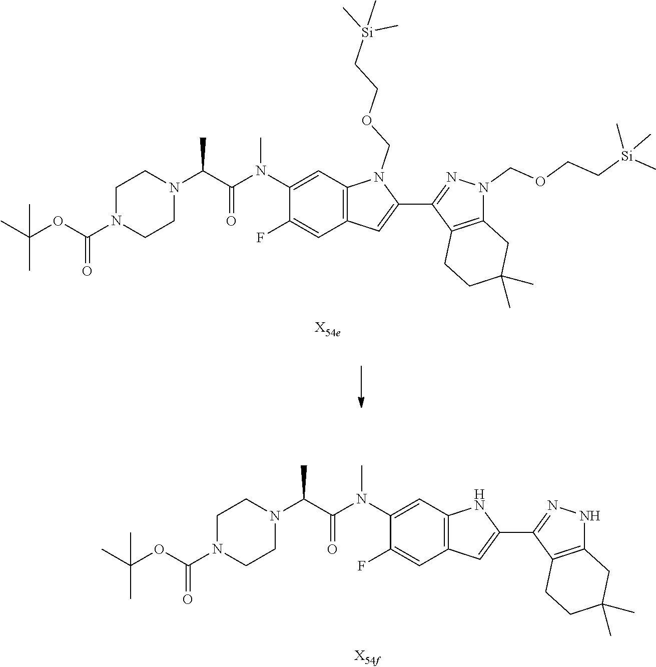

A mixture of tert-butyl (S)-4-(1-((2-(6,6-dimethyl-1-((2-(trimethylsilyl)ethoxy)methyl)-4,5,6,7-tetrahydro-1H-indazol-3-yl)-5-fluoro-1-((2-(trimethylsilyl)ethoxy)methyl)-1H-indol-6-yl)(methyl)amino)-1-oxopropan-2-yl)piperazine-1-carboxylate (31 mg, 0.04 mmol), ethylenediamine (0.15 mL, 0.14 g, 2.29 mmol) and tetra-n-butylammonium fluoride 1M solution in THE (0.38 mL, 0.38 mmol) in DMF (1.00 mL) was heated to 80° C. for 3 hours. The mixture was cooled. Water was added and the mixture was extracted twice with ethyl acetate. The combined organic layers were concentrated then purified by preparative TLC eluted with 10% MeOH/DCM to provide tert-butyl (S)-4-(1-((2-(6,6-dimethyl-4,5,6,7-tetrahydro-1H-indazol-3-yl)-5-fluoro-1H-indol-6-yl)(methyl)amino)-1-oxopropan-2-yl)piperazine-1-carboxylate (0.012 g, 57%). LCSM C₃₀H₄₁FN₆O₃ requires: 552, found: m/z=553 [M+H]⁺.

### Step 5: Synthesis of (S)—N-(2-(6,6-dimethyl-4,5,6,7-tetrahydro-1H-indazol-3-yl)-5-fluoro-1H-indol-6-yl)-N-methyl-2-(piperazin-1-yl)propanamide

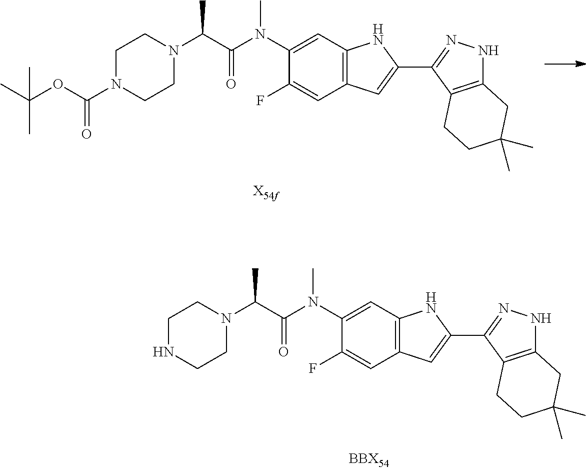 [SMILES: C[C@@H](C(=O)N(C)c1cc2[nH]c(-c3n[nH]c4c3CCC(C)(C)C4)cc2cc1F)N1CCN(C(=O)OC(C)(C)C)CC1 ; C[C@@H](C(=O)N(C)c1cc2[nH]c(-c3n[nH]c4c3CCC(C)(C)C4)cc2cc1F)N1CCNCC1]

tert-butyl (S)-4-(1-((2-(6,6-dimethyl-4,5,6,7-tetrahydro-1H-indazol-3-yl)-5-fluoro-1H-indol-6-yl)(methyl)amino)-1-oxopropan-2-yl)piperazine-1-carboxylate (12 mg, 0.02 mmol) was dissolved in DCM (1.00 mL) and TFA (1.00 mL). After 10 minutes, the mixture was concentrated to provide (S)—N-(2-(6,6-dimethyl-4,5,6,7-tetrahydro-1H-indazol-3-yl)-5-fluoro-1H-indol-6-yl)-N-methyl-2-(piperazin-1-yl)propenamide (9 mg, 100%). LCSM C₂₅H₃₃FN₆O requires: 452, found: m/z=453 [M+H]⁺.

### Step 6: Synthesis of (2S)—N-(2-(6,6-dimethyl-4,5,6,7-tetrahydro-1H-indazol-3-yl)-5-fluoro-1H-indol-6-yl)-2-(4-((1-(5-(2,6-dioxopiperidin-3-yl)pyridin-2-yl)piperidin-4-yl)methyl)piperazin-1-yl)-N-methylpropanamide

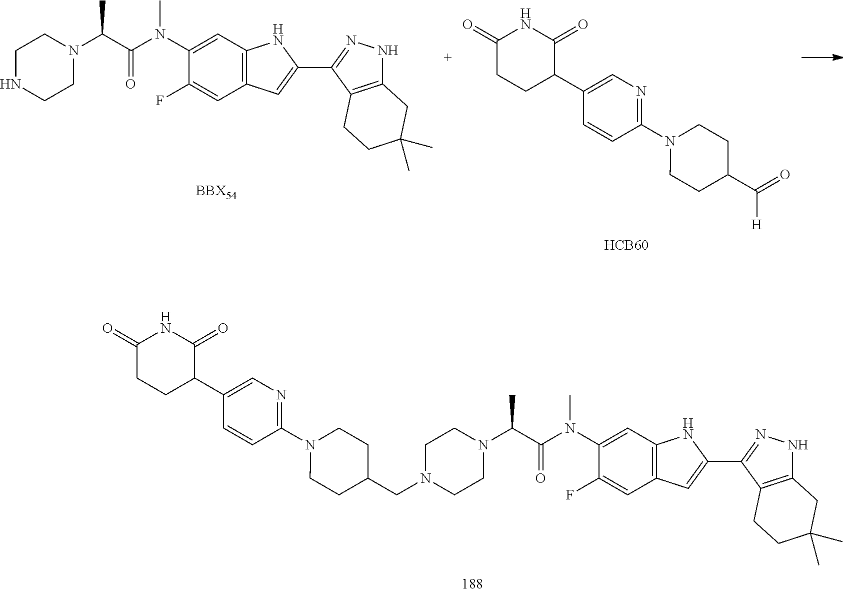 [SMILES: C[C@@H](C(=O)N(C)c1cc2[nH]c(-c3n[nH]c4c3CCC(C)(C)C4)cc2cc1F)N1CCNCC1 ; C[C@@H](C(=O)N(C)c1cc2[nH]c(-c3n[nH]c4c3CCC(C)(C)C4)cc2cc1F)N1CCN(CC2CCN(c3ccc(C4CCC(=O)NC4=O)cn3)CC2)CC1 ; "HCB60" ; O=CC1CCN(c2ccc(C3CCC(=O)NC3=O)cn2)CC1]

To a mixture of (S)—N-(2-(6,6-dimethyl-4,5,6,7-tetrahydro-1H-indazol-3-yl)-5-fluoro-1H-indol-6-yl)-N-methyl-2-(piperazin-1-yl)propanamide (BBX₅₄) (9 mg, 0.02 mmol) in DCM (1.00 mL) was added N,N-diisopropylethylamine (0.03 mL, 0.02 g, 0.16 mmol). 1-(5-(2,6-dioxopiperidin-3-yl)pyridin-2-yl)piperidine-4-carbaldehyde (HCB60) (6 mg, 0.02 mmol) was added followed by sodium triacetoxyborohydride (13 mg, 0.06 mmol). After 40 minutes, water was added. The mixture was extracted twice with DCM. The combined organic layers were concentrated then purified by preparative TLC to provide (2S)—N-(2-(6,6-dimethyl-4,5,6,7-tetrahydro-1H-indazol-3-yl)-5-fluoro-1H-indol-6-yl)-2-(4-((1-(5-(2,6-dioxopiperidin-3-yl)pyridin-2-yl)piperidin-4-yl)methyl)piperazin-1-yl)-N-methylpropanamide (0.0101 g, 67.1%). ¹H NMR (500 MHz, DMSO) δ 12.60 (s, 1H), 11.55-11.36 (m, 1H), 10.80 (s, 1H), 7.93 (d, J=2.5 Hz, 1H), 7.42 (d, J=10.6 Hz, 1H), 7.38-7.33 (m, 1H), 7.28 (s, 1H), 6.77 (d, J=8.9 Hz, 1H), 6.62 (s, 1H), 4.23 (d, J=13.0 Hz, 2H), 3.72 (dd, J=12.2, 4.9 Hz, 1H), 3.16 (d, J=13.8 Hz, 3H), 2.81-2.62 (m, 5H), 2.59-2.33 (m, 8H), 2.32-2.01 (m, 8H), 2.01-1.94 (m, 1H), 1.77-1.65 (m, 3H), 1.59 (t, J=6.4 Hz, 2H), 1.15-0.94 (m, 10H). LCSM C₄₁H₅₂FN₉O₃ requires: 737, found: m/z=738 [M+H]⁺.

### Example 316. 3-(6-((R)-4-((1-(2-(6,6-dimethyl-4,5,6,7-tetrahydro-1H-indazol-3-yl)-1H-indole-6-carbonyl)piperidin-4-yl)methyl)-3-methylpiperazin-1-yl)pyridin-3-yl)piperidine-2,6-dione (189)

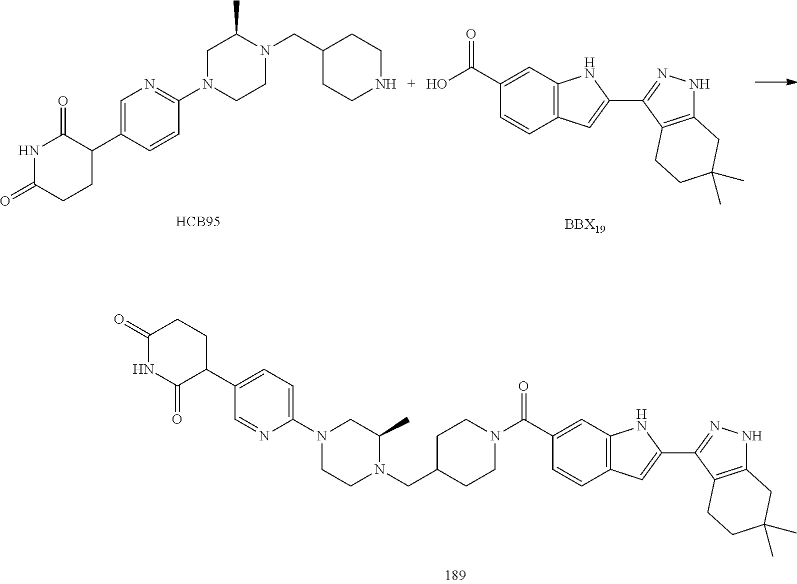

To a 20 mL vial was added (3RS)-3-{6-[(3R)-3-methyl-4-(piperidin-4-ylmethyl)piperazin-1-yl]pyridin-3-yl}piperidine-2,6-dione (HCB95) (20.70 mg, 0.05 mmol), 2-(6,6-dimethyl-1,4,5,7-tetrahydroindazol-3-yl)-1H-indole-6-carboxylic acid (BBX₁₉) (16.61 mg, 0.05 mmol), N,N-diisopropylethylamine (0.10 mL, 0.07 g, 0.57 mmol), and DMF (1.0 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (20.42 mg, 0.05 mmol) in DMF (0.4 mL). After 1 h, the reaction mixture was diluted with water. The product was extracted with DCM (3×), dried over MgSO4, then concentrated. The resulting residue was purified by column chromatography (4 g silica, 0-20% MeOH/DCM) to yield the title compound as a white solid (19.5 mg, 54%). LCMS: C₃₉H₄₈N₈O₃ requires: 676.4, found: m/z=677.6 [M+H]⁺. ¹H NMR (500 MHz, DMSO) δ 12.55 (s, 1H), 11.42 (s, 1H), 10.80 (s, 1H), 7.94 (d, J=2.5 Hz, 1H), 7.52 (d, J=8.1 Hz, 1H), 7.43 (s, 1H), 7.37 (dd, J=8.8, 2.5 Hz, 1H), 6.99 (d, J=8.1 Hz, 1H), 6.79 (d, J=8.9 Hz, 1H), 6.61 (s, 1H), 5.76 (s, 1H), 3.85 (d, J=12.1 Hz, 2H), 3.72 (dd, J=12.1, 4.9 Hz, 1H), 3.04 (t, J=10.6 Hz, 1H), 2.90 (d, J=10.9 Hz, 3H), 2.75 (t, J=10.8 Hz, 1H), 2.66 (dt, J=17.1, 6.2 Hz, 3H), 2.61-2.52 (m, 1H), 2.41 (s, 2H), 2.37 (s, 1H), 2.16 (s, 2H), 2.01-1.93 (m, 2H), 1.81 (s, 2H), 1.68 (s, 1H), 1.58 (t, J=6.4 Hz, 2H), 1.04 (d, J=6.1 Hz, 3H), 1.01 (s, 6H).

### Example 317. 3-(6-((S)-4-((1-(2-(6,6-dimethyl-4,5,6,7-tetrahydro-1H-indazol-3-yl)-1H-indole-6-carbonyl)piperidin-4-yl)methyl)-3-methylpiperazin-1-yl)pyridin-3-yl)piperidine-2,6-dione (190)

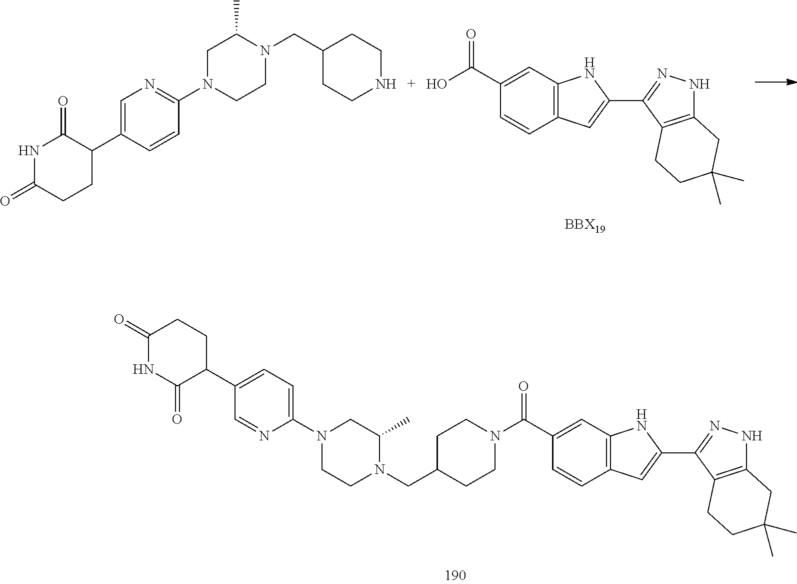

To a 20 mL vial was added (3RS)-3-{6-[(3S)-3-methyl-4-(piperidin-4-ylmethyl)piperazin-1-yl]pyridin-3-yl}piperidine-2,6-dione (23.00 mg, 0.06 mmol), 2-(6,6-dimethyl-1,4,5,7-tetrahydroindazol-3-yl)-1H-indole-6-carboxylic acid (BBX₁₉) (18.46 mg, 0.06 mmol), and DMF (1.0 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (22.69 mg, 0.06 mmol) in DMF (0.4 mL). After 1 h, the reaction mixture was diluted with water. The product was extracted with DCM (3×), dried over MgSO4, then concentrated. The resulting residue was purified by column chromatography (4 g silica, 0-20% MeOH/DCM) to yield the title compound as a white solid (11.1 mg, 27%). LCMS: C₃₉H₄₈N₈O₃ requires: 676.4, found: m/z=677.6 [M+H]⁺. ¹H NMR (500 MHz, DMSO) δ 12.56 (s, 1H), 11.42 (s, 1H), 10.80 (s, 1H), 7.94 (d, J=2.5 Hz, 1H), 7.52 (d, J=8.0 Hz, 1H), 7.43 (s, 1H), 7.37 (dd, J=8.8, 2.5 Hz, 1H), 6.99 (d, J=8.1 Hz, 1H), 6.79 (d, J=8.8 Hz, 1H), 6.61 (s, 1H), 3.85 (d, J=12.0 Hz, 2H), 3.72 (dd, J=12.2, 4.9 Hz, 1H), 3.05 (t, J=10.9 Hz, 1H), 2.90 (d, J=13.1 Hz, 2H), 2.80-2.69 (m, 1H), 2.67 (dt, J=12.1, 7.2 Hz, 3H), 2.62-2.52 (m, 1H), 2.40 (d, J=16.4 Hz, 3H), 2.17 (td, J=12.5, 8.4 Hz, 2H), 2.02-1.93 (m, 2H), 1.81 (s, 2H), 1.68 (s, 1H), 1.58 (t, J=6.3 Hz, 2H), 1.04 (d, J=6.2 Hz, 3H), 1.01 (s, 6H).

### Example 318. 3-(6-(4-((1-(2-(6,6-dimethyl-4,5,6,7-tetrahydro-1H-indazol-3-yl)-1H-indole-6-carbonyl)piperidin-4-yl)methyl)-1,4-diazepan-1-yl)pyridin-3-yl)piperidine-2,6-dione (191)

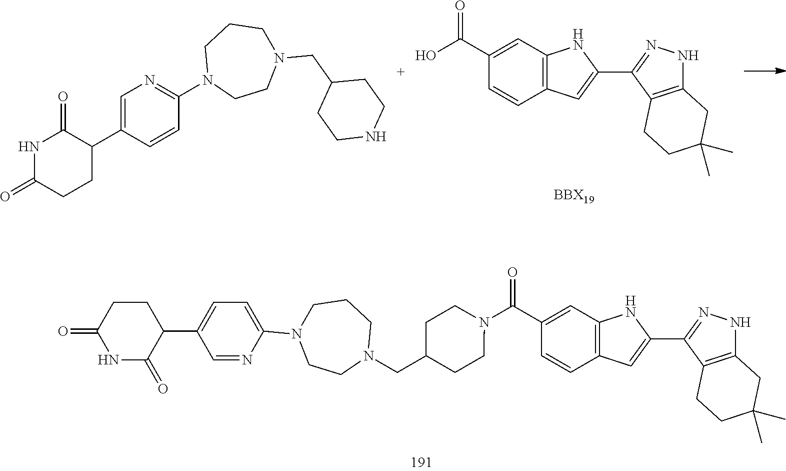 [SMILES: O=C1CCC(c2ccc(N3CCCN(CC4CCNCC4)CC3)nc2)C(=O)N1 ; CC1(C)CCc2c(-c3cc4ccc(C(=O)O)cc4[nH]3)n[nH]c2C1 ; CC1(C)CCc2c(-c3cc4ccc(C(=O)N5CCC(CN6CCCN(c7ccc(C8CCC(=O)NC8=O)cn7)CC6)CC5)cc4[nH]3)n[nH]c2C1]

To a 20 mL vial was added rac-(3R)-3-{6-[4-(piperidin-4-ylmethyl)-1,4-diazepan-1-yl]pyridin-3-yl}piperidine-2,6-dione (20.00 mg, 0.05 mmol), 2-(6,6-dimethyl-1,4,5,7-tetrahydroindazol-3-yl)-1H-indole-6-carboxylic acid (BBX₁₉) (16.05 mg, 0.05 mmol), N,N-diisopropylethylamine (0.10 mL, 0.07 g, 0.57 mmol), and DMF (1.0 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (19.73 mg, 0.05 mmol) in DMF (0.1 mL). After 1 h, the reaction mixture was diluted with water and extracted with DCM (3×). The combined organic layers were dried over MgSO4, then concentrated. The resulting residue was purified by column chromatography (4 g silica, 2-20% MeOH/DCM) to yield the title compound as a white solid (9.1 mg, 25%). LCMS: C₃₉H₄₈N₈O₃ requires: 676.4, found: m/z=677.5 [M+H]⁺. ¹H NMR (500 MHz, DMSO) δ 12.56 (s, 1H), 11.42 (s, 1H), 10.77 (s, 1H), 7.88 (s, 1H), 7.51 (d, J=8.1 Hz, 1H), 7.42 (s, 1H), 7.32 (d, J=8.4 Hz, 1H), 6.97 (d, J=8.2 Hz, 1H), 6.62-6.55 (m, 2H), 3.68 (s, 3H), 3.57 (d, J=6.5 Hz, 2H), 2.68 (s, 5H), 2.42 (s, 2H), 2.31 (s, 2H), 2.14 (d, J=12.6 Hz, 2H), 1.96 (s, 1H), 1.83 (s, 2H), 1.71 (s, 4H), 1.58 (t, J=6.3 Hz, 2H), 1.01 (s, 7H).

### Example 319. 3-(2-(4-((1-(2-(6,6-dimethyl-4,5,6,7-tetrahydro-1H-indazol-3-yl)-1H-indole-6-carbonyl)piperidin-4-yl)methyl)-1,4-diazepan-1-yl)pyridin-4-yl)piperidine-2,6-dione (192)

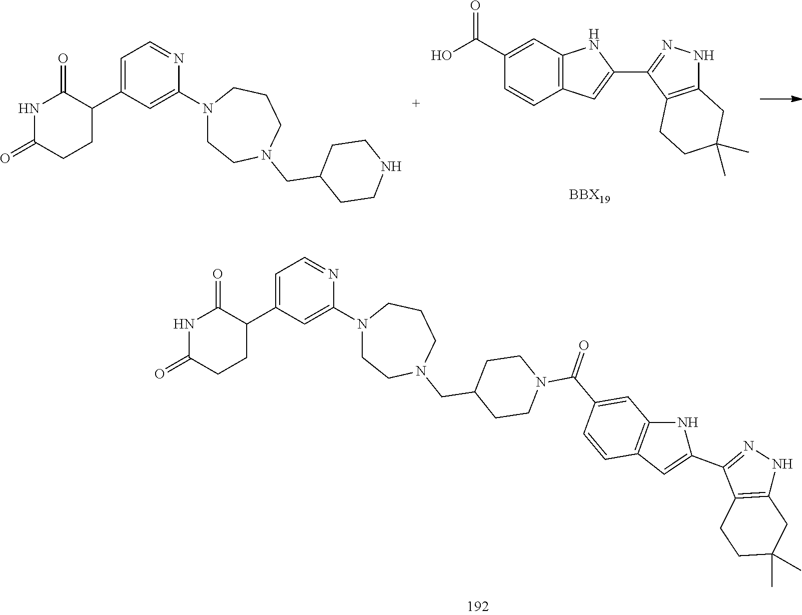 [SMILES: O=C1CCC(c2ccnc(N3CCCN(CC4CCNCC4)CC3)c2)C(=O)N1 ; CC1(C)CCc2c(-c3cc4ccc(C(=O)O)cc4[nH]3)n[nH]c2C1 ; CC1(C)CCc2c(-c3cc4ccc(C(=O)N5CCC(CN6CCCN(c7cc(C8CCC(=O)NC8=O)ccn7)CC6)CC5)cc4[nH]3)n[nH]c2C1]

To a 20 mL vial was added rac-(3R)-3-{2-[4-(piperidin-4-ylmethyl)-1,4-diazepan-1-yl]pyridin-4-yl}piperidine-2,6-dione hydrochloride (46.42 mg, 0.11 mmol), 2-(6,6-dimethyl-1,4,5,7-tetrahydroindazol-3-yl)-1H-indole-6-carboxylic acid (BBX₁₉) (34.03 mg, 0.11 mmol), N,N-diisopropylethylamine (0.19 mL, 0.14 g, 1.10 mmol), and DMF (1 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (41.83 mg, 0.11 mmol) in DMF (1 mL). The reaction mixture was diluted with sat. NaHCO₃ (aq), and the product was extracted with DCM (3×). The combined organic layers were dried over MgSO4, then concentrated. The resulting residue was purified by column chromatography (4 g silica, 0-20% MeOH/DCM) to yield the title compound as a white solid (35 mg, 47%). LCMS: C₃₉H₄₈N₈O₃ requires: 676.4, found: m/z=677.4 [M+H]⁺. ¹H NMR (500 MHz, DMSO) δ 12.55 (s, 1H), 11.41 (s, 1H), 10.84 (s, 1H), 7.97 (s, 1H), 7.52 (d, J=8.1 Hz, 1H), 7.42 (s, 1H), 6.97 (d, J=8.0 Hz, 1H), 6.61 (s, 1H), 6.46 (s, 1H), 6.38 (s, 1H), 3.74 (d, J=10.3 Hz, 1H), 3.67 (s, 2H), 3.58 (s, 2H), 3.29 (s, 1H), 3.10 (s, 1H), 2.68 (s, 4H), 2.41 (s, 2H), 2.30 (s, 2H), 2.20 (d, J=12.9 Hz, 2H), 2.02-1.94 (m, 1H), 1.82 (s, 2H), 1.71 (s, 4H), 1.58 (t, J=6.4 Hz, 2H), 1.17 (t, J=7.4 Hz, 2H), 1.01 (s, 6H).

### Example 320. 3-(2-((1-(2-(6,6-dimethyl-4,5,6,7-tetrahydro-1H-indazol-3-yl)-1H-indole-6-carbonyl)piperidin-4-yl)methyl)-1,2,3,4-tetrahydroisoquinolin-6-yl)piperidine-2,6-dione (193)

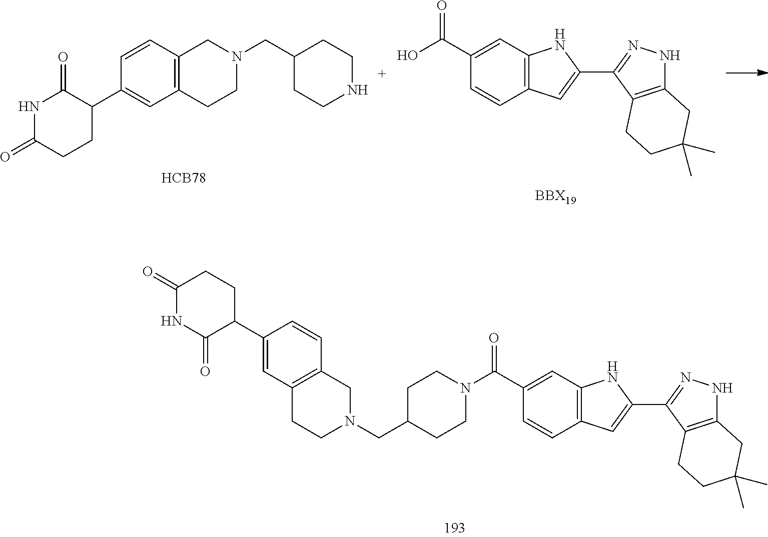

To a 1 dram vial was added rac-(3R)-3-[2-(piperidin-4-ylmethyl)-3,4-dihydro-1H-isoquinolin-6-yl]piperidine-2,6-dione dihydrochloride (HCB78, HCl) (30.00 mg, 0.07 mmol), 2-(6,6-dimethyl-1,4,5,7-tetrahydroindazol-3-yl)-1H-indole-6-carboxylic acid (BBX₁₉) (22.40 mg, 0.07 mmol), N,N-diisopropylethylamine (0.13 mL, 0.09 g, 0.72 mmol), and DMF (1 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (27.53 mg, 0.07 mmol) in DMF (0.5 mL). The reaction mixture was stirred for 3 h, then quenched with sat. NaHCO₃(aq). The product was extracted with DCM (3×), dried over MgSO4, then concentrated. The resulting residue was purified by FC (4 g silica, 0-15% MeOH/DCM) to yield the title compound as a white solid (37.8 mg, 78%). LCMS: C₃₈H₄₄N₆O₃ requires: 632.3, found: m/z=633.4 [M+H]⁺. ¹H NMR (500 MHz, DMSO) δ 12.55 (s, 1H), 11.42 (s, 1H), 10.80 (s, 1H), 7.52 (d, J=8.1 Hz, 1H), 7.43 (s, 1H), 7.05-6.90 (m, 3H), 6.60 (s, 1H), 3.79-3.73 (m, 1H), 3.53 (s, 2H), 2.79 (s, 2H), 2.69-2.59 (m, 5H), 2.41 (s, 2H), 2.21-2.11 (m, 1H), 2.08-1.88 (m, 1H), 1.77 (s, 2H), 1.58 (t, J=6.4 Hz, 2H), 1.28-1.04 (m, 3H), 1.01 (s, 6H).

### Example 321. 3-(2-((1-(2-(6,6-dimethyl-4,5,6,7-tetrahydro-1H-indazol-3-yl)-1H-indole-5-carbonyl)piperidin-4-yl)methyl)-1,2,3,4-tetrahydroisoquinolin-6-yl)piperidine-2,6-dione (194)

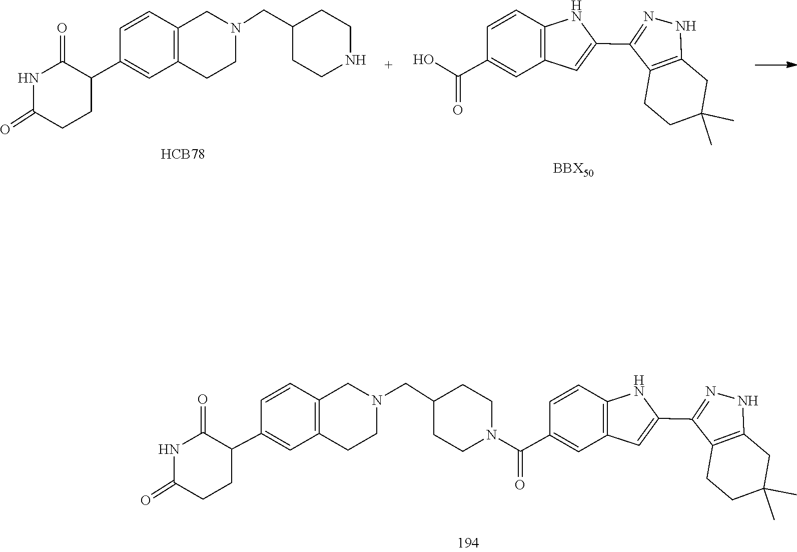 [SMILES: "HCB78" ; O=C1CCC(c2ccc3c(c2)CCN(CC2CCNCC2)C3)C(=O)N1 ; CC1(C)CCc2c(-c3cc4cc(C(=O)O)ccc4[nH]3)n[nH]c2C1 ; CC1(C)CCc2c(-c3cc4cc(C(=O)N5CCC(CN6CCc7cc(C8CCC(=O)NC8=O)ccc7C6)CC5)ccc4[nH]3)n[nH]c2C1]

To a 1 dram vial was added rac-(3R)-3-[2-(piperidin-4-ylmethyl)-3,4-dihydro-1H-isoquinolin-7-yl]piperidine-2,6-dione dihydrochloride (HCB78, HCl) (7.40 mg, 0.02 mmol), 2-(6,6-dimethyl-1,4,5,7-tetrahydroindazol-3-yl)-1H-indole-5-carboxylic acid (BBX₅₀) (5.52 mg, 0.02 mmol), N,N-diisopropylethylamine (0.03 mL, 0.02 g, 0.18 mmol), and DMF (0.25 mL). To the reaction mixture was added [(dimethylamino)({[1,2,3]triazolo[4,5-b]pyridin-3-yloxy})methylidene]dimethylazanium; hexafluoro-lambda5-phosphanuide (6.79 mg, 0.02 mmol) in DMF (0.5 mL). After 1 h, the reaction mixture was diluted with sat. NaHCO₃ (aq). The product was extracted with DCM (3×), dried over MgSO₄, then concentrated. The resulting residue was purified by FC (4 g silica, 0-20% MeOH/DCM) to yield the title compound as a white solid (8.2 mg, 70%). LCMS: C₃₈H₄₄N₆O₃ requires: 632.3, found: m/z=633.4 [M+H]⁺. ¹H NMR (500 MHz, DMSO-d6) δ 12.53 (s, 1H), 11.43 (s, 1H), 10.79 (s, 1H), 7.54 (s, 1H), 7.39 (d, J=8.3 Hz, 1H), 7.08 (d, J=8.3 Hz, 1H), 7.00 (d, J=7.9 Hz, 1H), 6.94 (d, J=10.9 Hz, 2H), 6.62 (s, 1H), 3.76 (d, J=10.0 Hz, 2H), 3.62 (dd, J=11.1, 5.9 Hz, 1H), 3.51 (d, J=8.1 Hz, 2H), 3.29 (s, 2H), 3.14 (dd, J=7.4, 4.1 Hz, 1H), 2.78 (s, 2H), 2.66 (d, J=10.7 Hz, 6H), 2.41 (s, 2H), 2.37-2.27 (m, 2H), 2.16 (d, J=12.3 Hz, 2H), 2.04-1.95 (m, 2H), 1.77 (s, 2H), 1.58 (t, J=6.3 Hz, 2H), 1.25 (q, J=7.3 Hz, 5H), 1.12 (d, J=11.4 Hz, 2H), 1.01 (s, 7H).

### Example 322. 3-(2-(2-(6,6-dimethyl-4,5,6,7-tetrahydro-1H-indazol-3-yl)-1H-indole-6-carbonyl)-1,2,3,4-tetrahydroisoquinolin-6-yl)piperidine-2,6-dione (195)

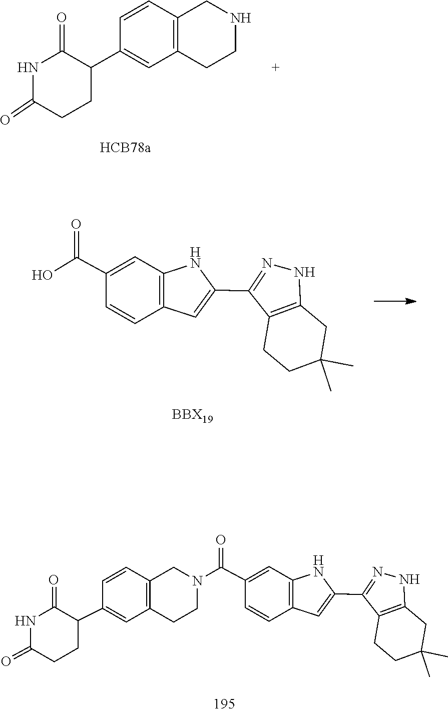 [SMILES: O=C1CCC(c2ccc3c(c2)CCNC3)C(=O)N1 ; CC1(C)CCc2c(-c3cc4ccc(C(=O)O)cc4[nH]3)n[nH]c2C1 ; CC1(C)CCc2c(-c3cc4ccc(C(=O)N5CCc6cc(C7CCC(=O)NC7=O)ccc6C5)cc4[nH]3)n[nH]c2C1]

To a 1 dram vial was added rac-(3R)-3-(1,2,3,4-tetrahydroisoquinolin-6-yl)piperidine-2,6-dione hydrochloride (HCB78a HCl) (7.10 mg, 0.03 mmol), 2-(6,6-dimethyl-1,4,5,7-tetrahydroindazol-3-yl)-1H-indole-6-carboxylic acid (BBX₁₉) (7.82 mg, 0.03 mmol), N,N-diisopropylethylamine (0.04 mL, 0.03 g, 0.25 mmol), and DMF (0.5 mL). To the reaction mixture was added [(dimethylamino)({[1,2,3]triazolo[4,5-b]pyridin-3-yloxy})methylidene]dimethylazanium; hexafluoro-lambda5-phosphanuide (9.62 mg, 0.03 mmol) in DMF (0.25 mL). The reaction mixture was stirred for 1 h, then quenched with sat. NaHCO₃(aq). The product was extracted with DCM (3×), dried over MgSO₄, then concentrated. The resulting residue was purified by FC (4 g silica, 0-10% MeOH/DCM) to yield the title compound as a white solid (11.2 mg, 79%). LCMS: C₃₂H₃₃N₅O₃ requires: 535.3, found: m/z=536.4 [M+H]⁺. ¹H NMR (500 MHz, DMSO-d6) δ 12.57 (s, 1H), 11.47 (s, 1H), 10.82 (s, 1H), 7.56 (d, J=8.1 Hz, 1H), 7.50 (s, 1H), 7.06 (d, J=16.1 Hz, 2H), 6.63 (s, 1H), 4.71 (s, 2H), 3.81 (dd, J=11.4, 4.9 Hz, 1H), 3.74 (s, 2H), 3.62 (s, 1H), 3.29 (s, 1H), 3.14 (s, 1H), 2.87 (q, J=5.1 Hz, 2H), 2.72-2.61 (m, 3H), 2.42 (s, 2H), 2.18 (dd, J=12.6, 8.5 Hz, 1H), 2.06-1.99 (m, 1H), 1.59 (t, J=6.4 Hz, 2H), 1.25 (q, J=7.2 Hz, 10H), 1.01 (s, 6H).

### Example 323. 3-(2-(2-(6,6-dimethyl-4,5,6,7-tetrahydro-1H-indazol-3-yl)-1H-indole-5-carbonyl)-1,2,3,4-tetrahydroisoquinolin-6-yl)piperidine-2,6-dione (196)

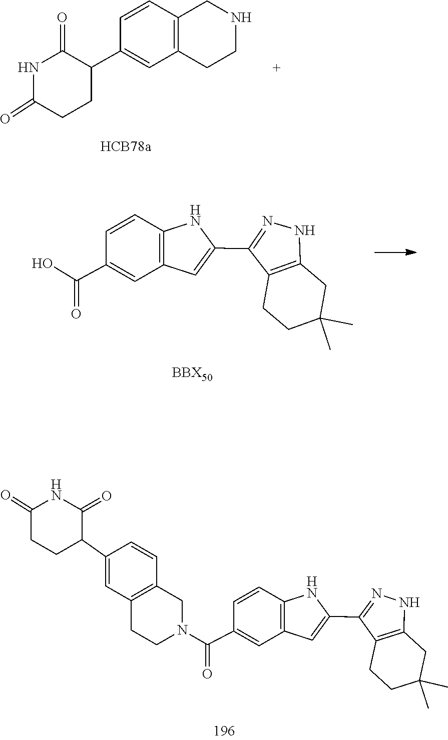 [SMILES: O=C1CCC(c2ccc3c(c2)CCNC3)C(=O)N1 ; CC1(C)CCc2c(-c3cc4cc(C(=O)O)ccc4[nH]3)n[nH]c2C1 ; CC1(C)CCc2c(-c3cc4cc(C(=O)N5CCc6cc(C7CCC(=O)NC7=O)ccc6C5)ccc4[nH]3)n[nH]c2C1]

To a 1 dram vial was added rac-(3R)-3-(1,2,3,4-tetrahydroisoquinolin-6-yl)piperidine-2,6-dione hydrochloride (HCB78a HCl) (7.70 mg, 0.03 mmol), 2-(6,6-dimethyl-1,4,5,7-tetrahydroindazol-3-yl)-1H-indole-5-carboxylic acid (BBX₅₀) (8.48 mg, 0.03 mmol), N,N-diisopropylethylamine (0.05 mL, 0.04 g, 0.27 mmol), and DMF (0.5 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (10.43 mg, 0.03 mmol) in DMF (0.25 mL). The reaction mixture was stirred for 1 h, then quenched with sat. NaHCO₃(aq). The product was extracted with DCM (3×), dried over MgSO₄, then concentrated.

The resulting residue was purified by FC (4 g silica, 0-10% MeOH/DCM) to yield the title compound as a white solid (14.4 mg, 88%). LCMS: C₃₂H₃₃N₅O₃ requires: 535.3, found: m/z=536.4 [M+H]⁺. ¹H NMR (500 MHz, DMSO-d6) δ 12.55 (s, 1H), 11.49 (s, 1H), 10.82 (s, 1H), 7.64 (s, 1H), 7.43 (d, J=8.2 Hz, 1H), 7.16 (d, J=8.3 Hz, 1H), 7.04 (s, 1H), 6.64 (s, 1H), 4.72 (s, 2H), 3.81 (dd, J=11.5, 5.0 Hz, 1H), 3.74 (s, 2H), 3.62 (td, J=6.6, 3.9 Hz, 1H), 3.29 (s, 1H), 3.15 (dt, J=7.4, 4.4 Hz, 1H), 2.87 (d, J=6.0 Hz, 2H), 2.72-2.61 (m, 3H), 2.42 (s, 2H), 2.24-2.13 (m, 1H), 2.06-1.98 (m, 1H), 1.58 (t, J=6.3 Hz, 2H), 1.25 (q, J=7.2 Hz, 10H), 1.01 (s, 6H).

### Example 324. 3-(2-((1-(2-(6,6-dimethyl-4,5,6,7-tetrahydro-1H-indazol-3-yl)-1H-indole-6-carbonyl)piperidin-4-yl)methyl)-1,2,3,4-tetrahydroisoquinolin-7-yl)piperidine-2,6-dione (197)

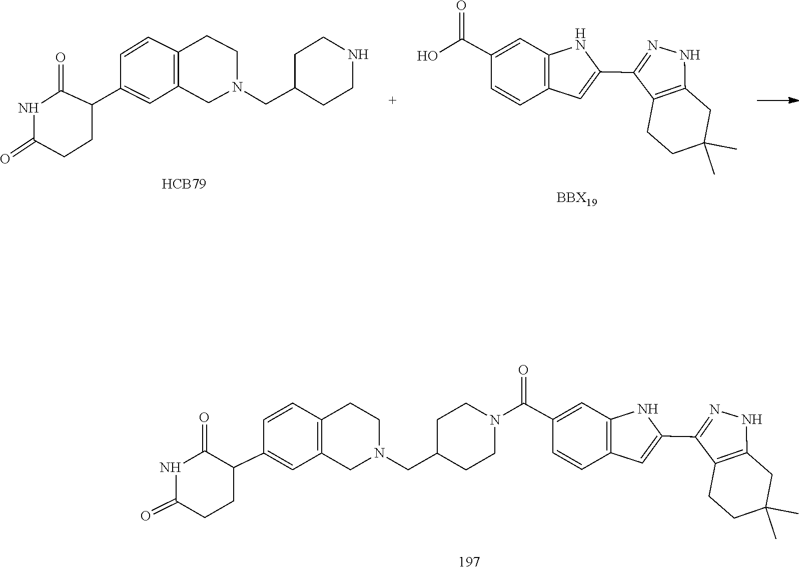 [SMILES: "HCB79" ; O=C1CCC(c2ccc3c(c2)CN(CC2CCNCC2)CC3)C(=O)N1 ; CC1(C)CCc2c(-c3cc4ccc(C(=O)O)cc4[nH]3)n[nH]c2C1 ; CC1(C)CCc2c(-c3cc4ccc(C(=O)N5CCC(CN6CCc7ccc(C8CCC(=O)NC8=O)cc7C6)CC5)cc4[nH]3)n[nH]c2C1]

To a 1 dram vial was added rac-(3R)-3-[2-(piperidin-4-ylmethyl)-3,4-dihydro-1H-isoquinolin-7-yl]piperidine-2,6-dione dihydrochloride (HCB79) (30.00 mg, 0.07 mmol), 2-(6,6-dimethyl-1,4,5,7-tetrahydroindazol-3-yl)-1H-indole-6-carboxylic acid (BBX₁₉) (22.40 mg, 0.07 mmol), N,N-diisopropylethylamine (0.13 mL, 0.09 g, 0.72 mmol), and DMF (1 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (27.53 mg, 0.07 mmol) in DMF (0.5 mL). The reaction mixture was stirred for 3 h, then quenched with sat. NaHCO₃ (aq). The product was extracted with DCM (3×), dried over MgSO₄, then concentrated. The resulting residue was purified by FC (4 g silica, 0-15% MeOH/DCM) to yield the title compound as a white solid (20.2 mg, 43%). LCMS: C₃₈H₄₄N₆O₃ requires: 632.3, found: m/z=633.5 [M+H]⁺. ¹H NMR (500 MHz, DMSO-d6) δ 12.57 (s, 1H), 11.43 (s, 1H), 10.82 (s, 1H), 7.53 (d, J=8.2 Hz, 1H), 7.45 (s, 1H), 7.08 (s, 1H), 7.00 (d, J=8.0 Hz, 2H), 6.92 (s, 1H), 6.62 (s, 1H), 3.79 (dd, J=11.5, 5.0 Hz, 1H), 2.83 (s, 3H), 2.68 (s, 3H), 2.43 (s, 2H), 2.16 (t, J=10.8 Hz, 1H), 2.09-1.91 (m, 1H), 1.79 (s, 2H), 1.59 (t, J=6.3 Hz, 2H), 1.27-1.05 (m, 3H), 1.02 (s, 5H).

### Example 325. 3-(2-((1-(2-(6,6-dimethyl-4,5,6,7-tetrahydro-1H-indazol-3-yl)-1H-indole-5-carbonyl)piperidin-4-yl)methyl)-1,2,3,4-tetrahydroisoquinolin-7-yl)piperidine-2,6-dione (198)

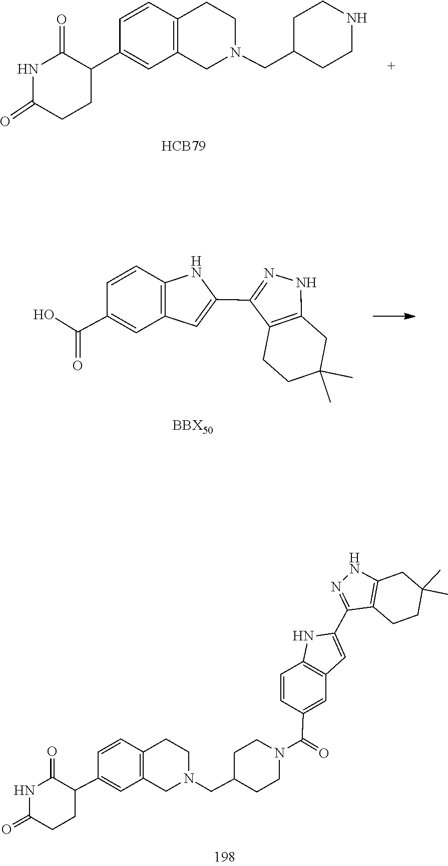 [SMILES: "HCB79" ; O=C1CCC(c2ccc3c(c2)CN(CC2CCNCC2)CC3)C(=O)N1 ; CC1(C)CCc2c(-c3cc4cc(C(=O)O)ccc4[nH]3)n[nH]c2C1 ; CC1(C)CCc2c(-c3cc4cc(C(=O)N5CCC(CN6CCc7ccc(C8CCC(=O)NC8=O)cc7C6)CC5)ccc4[nH]3)n[nH]c2C1]

To a 1 dram vial was added rac-(3R)-3-[2-(piperidin-4-ylmethyl)-3,4-dihydro-1H-isoquinolin-7-yl]piperidine-2,6-dione dihydrochloride (HCB79) (7.40 mg, 0.02 mmol), 2-(6,6-dimethyl-1,4,5,7-tetrahydroindazol-3-yl)-1H-indole-5-carboxylic acid (BBX₅₀) (5.52 mg, 0.02 mmol), N,N-diisopropylethylamine (0.03 mL, 0.02 g, 0.18 mmol), and DMF (0.25 mL). To the reaction mixture was added [(dimethylamino)({[1,2,3]triazolo[4,5-b]pyridin-3-yloxy})methylidene]dimethylazanium; hexafluoro-lambda5-phosphanuide (6.79 mg, 0.02 mmol) in DMF (0.5 mL). After 1 h, the reaction mixture was diluted with sat. NaHCO₃ (aq). The product was extracted with DCM (3×), dried over MgSO4, then concentrated. the resulting residue was purified by FC (4 g silica, 0-20% MeOH/DCM) to yield the title compound as a white solid (6.2 mg, 57%). LCMS: C₃₈H₄₄N₆O₃ requires: 632.3, found: m/z=633.4 [M+H]⁺. ¹H NMR (500 MHz, DMSO-d6) δ 12.53 (s, 1H), 11.43 (s, 1H), 10.80 (s, 1H), 7.54 (s, 1H), 7.39 (d, J=8.3 Hz, 1H), 7.11-7.02 (m, 2H), 6.96 (s, 1H), 6.88 (s, 1H), 6.62 (s, 1H), 3.77 (s, 2H), 3.68-3.58 (m, 1H), 3.51 (s, 2H), 3.29 (s, 2H), 3.14 (dd, J=7.4, 4.3 Hz, 1H), 2.78 (s, 2H), 2.65 (d, J=15.4 Hz, 5H), 2.41 (s, 2H), 2.34 (s, 1H), 2.29 (s, 1H), 2.16 (s, 1H), 2.05-1.98 (m, 1H), 1.97 (s, 1H), 1.76 (s, 2H), 1.58 (t, J=6.3 Hz, 2H), 1.25 (q, J=5.4 Hz, 6H), 1.13 (s, 3H), 1.01 (s, 6H).

### Example 326. 3-(1-((1-(2-(6,6-dimethyl-4,5,6,7-tetrahydro-1H-indazol-3-yl)-1H-indole-6-carbonyl)piperidin-4-yl)methyl)-1,2,3,4-tetrahydroquinolin-6-yl)piperidine-2,6-dione (199)

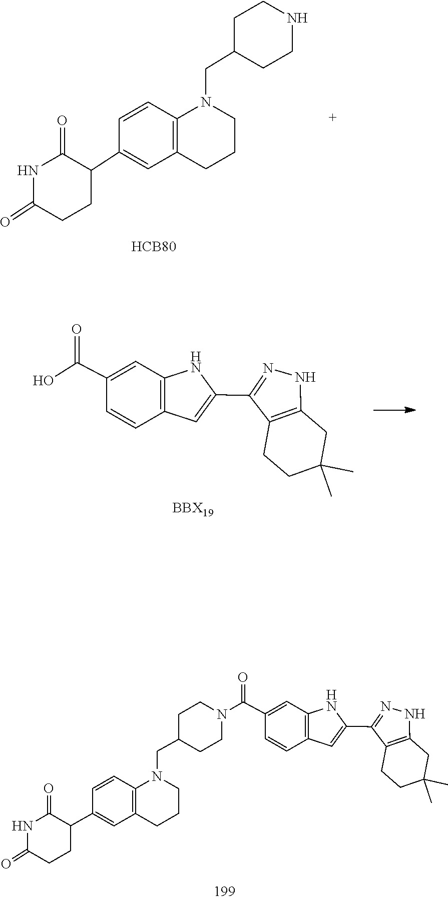 [SMILES: "HCB80" ; O=C1CCC(c2ccc3c(c2)CCCN3CC2CCNCC2)C(=O)N1 ; CC1(C)CCc2c(-c3cc4ccc(C(=O)O)cc4[nH]3)n[nH]c2C1 ; CC1(C)CCc2c(-c3cc4ccc(C(=O)N5CCC(CN6CCCc7cc(C8CCC(=O)NC8=O)ccc76)CC5)cc4[nH]3)n[nH]c2C1]

To a 1 dram vial was added rac-(3R)-3-[2-(piperidin-4-ylmethyl)-3,4-dihydro-1H-isoquinolin-6-yl]piperidine-2,6-dione dihydrochloride (HCB80) (7.40 mg, 0.02 mmol), 2-(6,6-dimethyl-1,4,5,7-tetrahydroindazol-3-yl)-1H-indole-5-carboxylic acid (BBX₁₉) (5.52 mg, 0.02 mmol), N,N-diisopropylethylamine (0.03 mL, 0.02 g, 0.18 mmol), and DMF (0.25 mL). To the reaction mixture was added [(dimethylamino)({[1,2,3]triazolo[4,5-b]pyridin-3-yloxy})methylidene]dimethylazanium; hexafluoro-lambda5-phosphanuide (6.79 mg, 0.02 mmol) in DMF (0.5 mL). After 1 h, the reaction mixture was diluted with sat. NaHCO₃ (aq). The product was extracted with DCM (3×), dried over MgSO₄, then concentrated. The resulting residue was purified by FC (4 g silica, 0-20% MeOH/DCM) to yield the title compound as a white solid (8.2 mg, 70%). LCMS: C₃₈H₄₄N₆O₃ requires: 632.3, found: m/z=633.4 [M+H]⁺. ¹H NMR (500 MHz, DMSO-d6) δ 12.57 (s, 1H), 11.45 (s, 1H), 10.73 (s, 1H), 8.16 (s, 1H), 7.53 (d, J=8.1 Hz, 1H), 7.45 (s, 1H), 7.00 (d, J=8.1 Hz, 1H), 6.79 (dd, J=8.3, 2.3 Hz, 1H), 6.70 (d, J=2.3 Hz, 1H), 6.62 (s, 1H), 6.51 (d, J=8.4 Hz, 1H), 3.62 (dq, J=10.6, 6.3 Hz, 3H), 3.30 (d, J=6.4 Hz, 5H), 3.15 (dd, J=12.3, 7.3 Hz, 4H), 2.73-2.56 (m, 5H), 2.49-2.41 (m, 4H), 2.09 (s, 1H), 1.99 (dd, J=13.0, 6.3 Hz, 3H), 1.87-1.81 (m, 2H), 1.73 (s, 3H), 1.59 (t, J=6.3 Hz, 2H), 1.26 (q, J=7.4 Hz, 12H), 1.21 (s, 1H), 1.02 (s, 6H).

### Example 327. 3-(1-((1-(2-(6,6-dimethyl-4,5,6,7-tetrahydro-1H-indazol-3-yl)-1H-indole-5-carbonyl)piperidin-4-yl)methyl)-1,2,3,4-tetrahydroquinolin-6-yl)piperidine-2,6-dione (200)

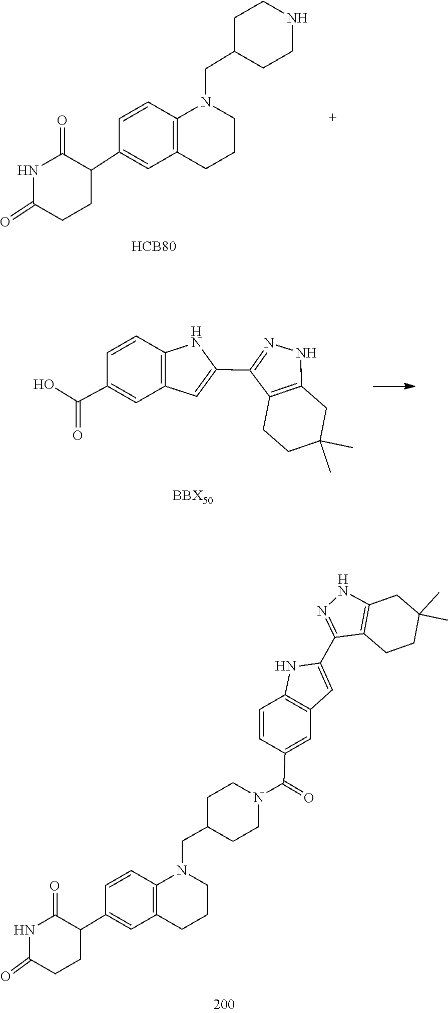 [SMILES: "HCB80" ; O=C1CCC(c2ccc3c(c2)CCCN3CC2CCNCC2)C(=O)N1 ; CC1(C)CCc2c(-c3cc4cc(C(=O)O)ccc4[nH]3)n[nH]c2C1 ; CC1(C)CCc2c(-c3cc4cc(C(=O)N5CCC(CN6CCCc7cc(C8CCC(=O)NC8=O)ccc76)CC5)ccc4[nH]3)n[nH]c2C1]

To a 1 dram vial was added rac-(3R)-3-[1-(piperidin-4-ylmethyl)-3,4-dihydro-2H-quinolin-6-yl]piperidine-2,6-dione dihydrochloride (HCB80) (7.50 mg, 0.02 mmol), 2-(6,6-dimethyl-1,4,5,7-tetrahydroindazol-3-yl)-1H-indole-5-carboxylic acid (BBX₅₀) (5.60 mg, 0.02 mmol), N,N-diisopropylethylamine (0.03 mL, 0.02 g, 0.18 mmol), and DMF (0.25 mL). To the reaction mixture was added a solution of [(dimethylamino)({[1,2,3]triazolo[4,5-b]pyridin-3-yloxy})methylidene]dimethylazanium; hexafluoro-lambda5-phosphanuide (6.88 mg, 0.02 mmol) in DMF (0.5 mL). After 1 h, the reaction mixture was diluted with sat. NaHCO₃(aq). The product was extracted with DCM (3×), dried over MgSO4, then concentrated. The resulting residue was purified by FC (4 g silica, 0-20% MeOH/DCM) to yield the title compound as a white solid (1.9 mg, 14%). LCMS: C₃₈H₄₄N₆O₃ requires: 632.3, found: m/z=633.5 [M+H]⁺. ¹H NMR (500 MHz, DMSO) δ 12.54 (s, 1H), 11.44 (s, 1H), 10.71 (s, 1H), 7.55 (s, 1H), 7.40 (d, J=8.2 Hz, 1H), 7.09 (d, J=8.4 Hz, 1H), 6.81-6.75 (m, 1H), 6.69 (d, J=2.3 Hz, 1H), 6.62 (s, 1H), 6.50 (d, J=8.4 Hz, 1H), 3.59 (dd, J=10.6, 5.0 Hz, 1H), 3.28 (d, J=6.5 Hz, 5H), 3.12 (d, J=7.0 Hz, 2H), 2.66 (d, J=8.4 Hz, 4H), 2.59 (td, J=11.0, 5.3 Hz, 1H), 2.48-2.41 (m, 1H), 2.41 (s, 2H), 2.12-1.93 (m, 1H), 1.85-1.80 (m, 3H), 1.70 (s, 2H), 1.58 (t, J=6.5 Hz, 2H), 1.47 (s, 1H), 1.23 (s, 3H), 1.20 (s, 2H), 1.01 (s, 7H).

### Example 328. (4aS,5aR)-N-(1-(1-((1-(3-(2,4-dioxotetrahydropyrimidin-1(2H)-yl)-4-fluorobenzoyl)piperidin-4-yl)methyl)piperidin-4-yl)-1H-pyrazol-4-yl)-5,5-difluoro-5a-methyl-1,4,4a,5,5a,6-hexahydrocyclopropa[f]indazole-3-carboxamide (201)

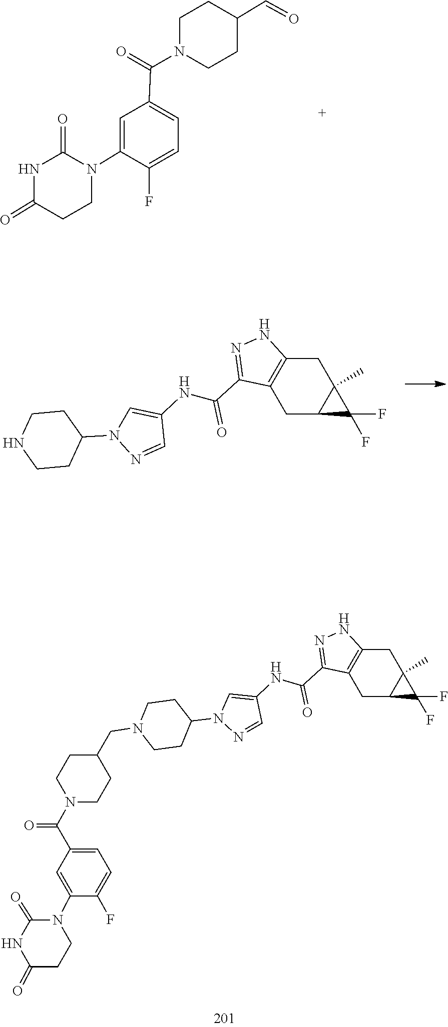 [SMILES: O=CC1CCN(C(=O)c2ccc(F)c(N3CCC(=O)NC3=O)c2)CC1 ; C[C@@]12Cc3[nH]nc(C(=O)Nc4cnn(C5CCNCC5)c4)c3C[C@@H]1C2(F)F ; C[C@@]12Cc3[nH]nc(C(=O)Nc4cnn(C5CCN(CC6CCN(C(=O)c7ccc(F)c(N8CCC(=O)NC8=O)c7)CC6)CC5)c4)c3C[C@@H]1C2(F)F]

To a 2 dram vial was added 1-[3-(2,4-dioxo-1,3-diazinan-1-yl)-4-fluorobenzoyl]piperidine-4-carbaldehyde (20.19 mg, 0.06 mmol), (4aS,5aR)-5,5-difluoro-5a-methyl-N-[1-(piperidin-4-yl)pyrazol-4-yl]-1H,4H,4aH,6H-cyclopropa[f]indazole-3-carboxamide hydrochloride (12.00 mg, 0.03 mmol), triethylamine (0.04 mL, 0.03 g, 0.29 mmol), DMSO (0.75 mL), and DCM (0.75 mL). To the reaction mixture was added sodium triacetoxyborohydride (30.80 mg, 0.15 mmol). After stirring the reaction mixture for 16 h, the reaction mixture was quenched with sat. NaHCO₃ (aq), and the product was extracted with DCM (3×), dried over MgSO4, then concentrated. The resulting residue was purified by FC (4 g silica, 0-15% MeOH/DCM) to yield the title compound as a white solid (11.4 mg, 53%). LCMS: C₃₅H₄₀F₃N₉O₄ requires: 707.3, found: m/z=708.6 [M+H]⁺. ¹H NMR (500 MHz, DMSO-d6) δ 12.96 (s, 1H), 10.52 (s, 1H), 10.10 (s, 1H), 7.99 (s, 1H), 7.62 (s, 1H), 7.52-7.47 (m, 1H), 7.40-7.33 (m, 2H), 4.44 (s, 1H), 4.08 (s, 1H), 3.76 (t, J=6.6 Hz, 2H), 3.61 (s, 1H), 3.06 (d, J=9.3 Hz, 3H), 3.01 (s, 1H), 2.91 (d, J=11.0 Hz, 2H), 2.81 (d, J=17.4 Hz, 1H), 2.73 (t, J=6.6 Hz, 2H), 2.18 (d, J=7.0 Hz, 2H), 2.04 (t, J=11.3 Hz, 2H), 1.93 (d, J=15.4 Hz, 3H), 1.90-1.84 (m, 1H), 1.78 (d, J=17.3 Hz, 5H), 1.68 (s, 1H), 1.35 (s, 3H), 1.23 (s, 1H), 1.08 (q, J=11.2 Hz, 2H).

### Example 329. 1-(5-(4-((4-(2-(6,6-dimethyl-4,5,6,7-tetrahydro-1H-indazol-3-yl)-1H-indole-6-carbonyl)piperazin-1-yl)methyl)piperidine-1-carbonyl)-2-fluorophenyl)dihydropyrimidine-2,4(1H,3H)-dione (202)

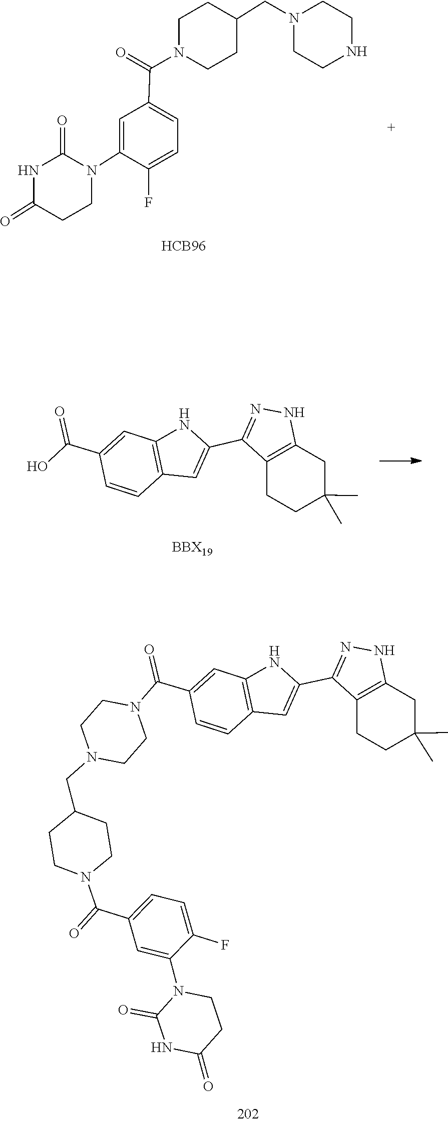 [SMILES: "HCB96" ; O=C1CCN(c2cc(C(=O)N3CCC(CN4CCNCC4)CC3)ccc2F)C(=O)N1 ; CC1(C)CCc2c(-c3cc4ccc(C(=O)O)cc4[nH]3)n[nH]c2C1 ; CC1(C)CCc2c(-c3cc4ccc(C(=O)N5CCN(CC6CCN(C(=O)c7ccc(F)c(N8CCC(=O)NC8=O)c7)CC6)CC5)cc4[nH]3)n[nH]c2C1]

To a 1 dram vial was added 1-{2-fluoro-5-[4-(piperazin-1-ylmethyl)piperidine-1-carbonyl]phenyl}-1,3-diazinane-2,4-dione (HCB96) (15.0 mg, 0.04 mmol), 2-(6,6-dimethyl-1,4,5,7-tetrahydroindazol-3-yl)-1H-indole-6-carboxylic acid (BBX₁₉) (11.1 mg, 0.04 mmol), N,N-diisopropylethylamine (0.06 mL, 0.05 g, 0.36 mmol), and DMF (1.0 mL). To the reaction mixture was added [(dimethylamino)({[1,2,3]triazolo[4,5-b]pyridin-3-yloxy})methylidene]dimethylazanium; hexafluoro-lambda5-phosphanuide (13.7 mg, 0.04 mmol). The reaction mixture was stirred for 1 h, then quenched with sat. NaHCO₃(aq). The product was extracted with DCM (3×), dried over MgSO4, then concentrated. The resulting residue was purified by FC (4 g silica, 0-10% MeOH/DCM) to yield the title compound as a white solid (6.7 mg, 26%). LCMS: C₃₉H₄₅FN₈O₄ requires: 708.4, found: m/z=709.6 [M+H]⁺. ¹H NMR (500 MHz, DMSO) δ 12.56 (s, 1H), 11.44 (s, 1H), 10.52 (s, 1H), 7.51 (dd, J=16.8, 7.6 Hz, 2H), 7.44 (s, 1H), 7.37 (d, J=7.5 Hz, 2H), 6.99 (d, J=7.8 Hz, 1H), 6.61 (s, 1H), 4.43 (s, 1H), 3.74 (d, J=7.1 Hz, 2H), 3.52 (s, 5H), 3.29 (s, 2H), 3.04 (s, 1H), 2.76-2.66 (m, 5H), 2.41 (s, 2H), 2.37 (s, 4H), 2.19 (s, 2H), 1.82 (s, 2H), 1.68 (s, 1H), 1.58 (s, 2H), 1.23 (s, 2H), 1.09 (d, J=12.4 Hz, 2H), 1.01 (s, 6H).

### Example 330. 1-(5-(4-((4-(2-(6,6-dimethyl-4,5,6,7-tetrahydro-1H-indazol-3-yl)-1H-indole-5-carbonyl)piperazin-1-yl)methyl)piperidine-1-carbonyl)-2-fluorophenyl)dihydropyrimidine-2,4(1H,3H)-dione (203)

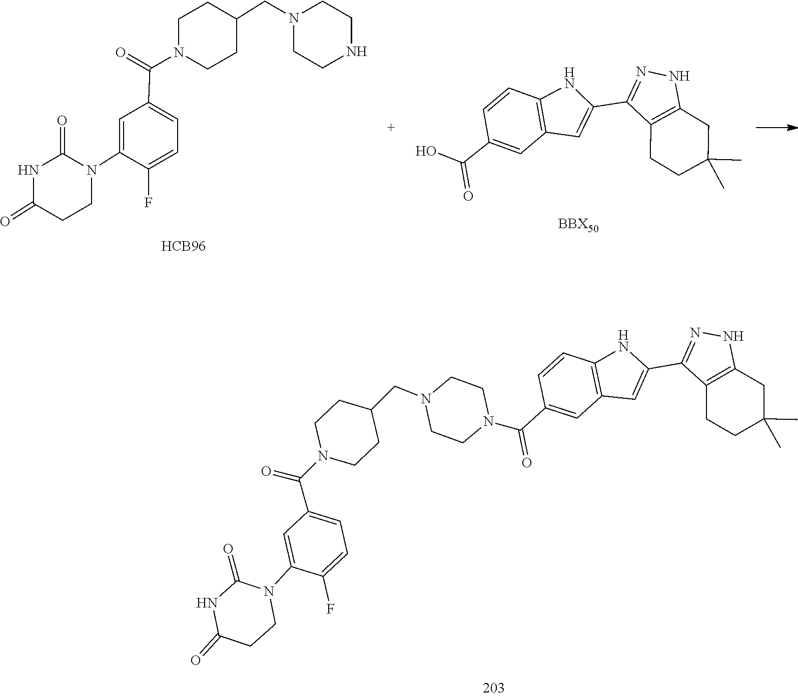 [SMILES: "HCB96" ; O=C1CCN(c2cc(C(=O)N3CCC(CN4CCNCC4)CC3)ccc2F)C(=O)N1 ; CC1(C)CCc2c(-c3cc4cc(C(=O)N5CCN(CC6CCN(C(=O)c7ccc(F)c(N8CCC(=O)NC8=O)c7)CC6)CC5)ccc4[nH]3)n[nH]c2C1 ; CC1(C)CCc2c(-c3cc4cc(C(=O)O)ccc4[nH]3)n[nH]c2C1]

To a 1 dram vial was added 1-{2-fluoro-5-[4-(piperazin-1-ylmethyl)piperidine-1-carbonyl]phenyl}-1,3-diazinane-2,4-dione (HCB96) (15.0 mg, 0.04 mmol), 2-(6,6-dimethyl-1,4,5,7-tetrahydroindazol-3-yl)-1H-indole-5-carboxylic acid (BBX₅₀) (11.1 mg, 0.04 mmol), N,N-diisopropylethylamine (0.06 mL, 0.05 g, 0.36 mmol), and DMF (1.0 mL). To the reaction mixture was added [(dimethylamino)({[1,2,3]triazolo[4,5-b]pyridin-3-yloxy})methylidene]dimethylazanium; hexafluoro-lambda5-phosphanuide (13.7 mg, 0.04 mmol). The reaction mixture was stirred for 1 h, then quenched with sat. NaHCO₃ (aq). The product was extracted with DCM (3×), dried over MgSO₄, then concentrated. The resulting residue was purified by FC (4 g silica, 0-10% MeOH/DCM) to yield the title compound as a white solid (6.2 mg, 21%). LCMS: C₃₉H₄₅FN₈O₄ requires: 708.4, found: m/z=709.6 [M+H]⁺. ¹H NMR (500 MHz, DMSO-d6) δ 12.54 (s, 1H), 11.45 (s, 1H), 10.52 (s, 2H), 7.56 (s, 1H), 7.52-7.46 (m, 2H), 7.37 (d, J=7.8 Hz, 5H), 7.09 (d, J=8.3 Hz, 1H), 6.62 (s, 1H), 4.44 (s, 2H), 3.75 (t, J=7.1 Hz, 4H), 3.61 (s, 3H), 3.52 (s, 5H), 3.14 (s, 1H), 3.04 (s, 3H), 2.76-2.69 (m, 4H), 2.67 (s, 3H), 2.41 (s, 3H), 2.36 (s, 7H), 2.33 (s, 1H), 2.27 (s, 2H), 2.17 (d, J=16.2 Hz, 5H), 1.97 (s, 2H), 1.82 (s, 5H), 1.67 (s, 2H), 1.58 (s, 2H), 1.25 (q, J=6.7 Hz, 10H), 1.08 (d, J=12.6 Hz, 6H), 1.01 (s, 5H).

### Example 331. (4aS,5aR)-N-(1-((1-((1-(3-(2,4-dioxotetrahydropyrimidin-1(2H)-yl)-4-fluorobenzoyl)piperidin-4-yl)methyl)piperidin-4-yl)methyl)-1H-pyrazol-4-yl)-5,5-difluoro-5a-methyl-1,4,4a,5,5a,6-hexahydrocyclopropa[f]indazole-3-carboxamide (204)

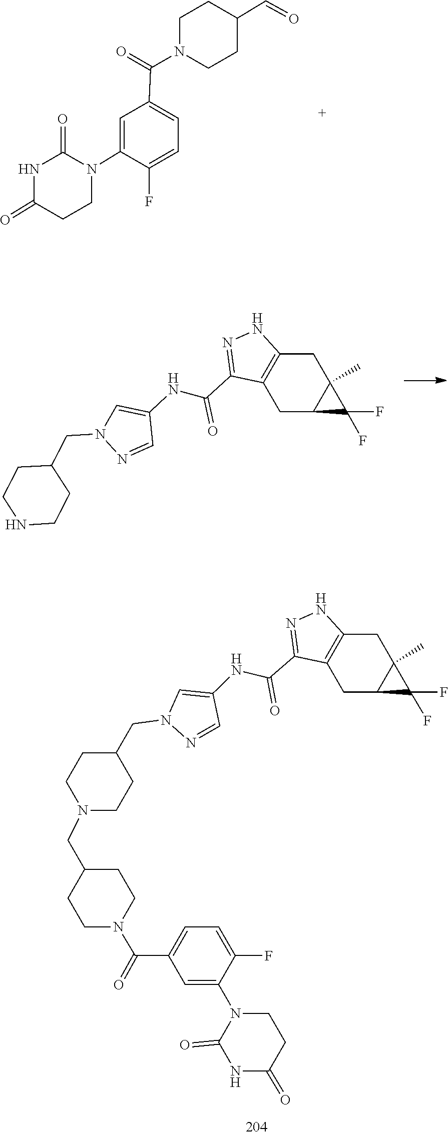 [SMILES: O=CC1CCN(C(=O)c2ccc(F)c(N3CCC(=O)NC3=O)c2)CC1 ; C[C@@]12Cc3[nH]nc(C(=O)Nc4cnn(CC5CCNCC5)c4)c3C[C@@H]1C2(F)F ; C[C@@]12Cc3[nH]nc(C(=O)Nc4cnn(CC5CCN(CC6CCN(C(=O)c7ccc(F)c(N8CCC(=O)NC8=O)c7)CC6)CC5)c4)c3C[C@@H]1C2(F)F]

To a 2 dram vial was added 1-[3-(2,4-dioxo-1,3-diazinan-1-yl)-4-fluorobenzoyl]piperidine-4-carbaldehyde (10.4 mg, 0.03 mmol), (4aS,5aR)-5,5-difluoro-5a-methyl-N-[1-(piperidin-4-ylmethyl)pyrazol-4-yl]-1H,4H,4aH,6H-cyclopropa[f]indazole-3-carboxamide hydrochloride (12.8 mg, 0.03 mmol), triethylamine (0.04 mL, 0.03 g, 0.29 mmol), DMSO (0.5 mL), and DCM (0.5 mL). To the reaction mixture was added sodium triacetoxyborohydride (19.1 mg, 0.09 mmol). After stirring the reaction mixture for 16 h, the reaction mixture was quenched with sat. NaHCO₃(aq), and the product was extracted with DCM (3×), dried over MgSO₄, then concentrated. The resulting residue was purified by FC (4 g silica, 0-15% MeOH/DCM) to yield the title compound as a white solid (13.4 mg, 62%). LCMS: C₃₆H₄₂F₃N₉O₄ requires: 721.3, found: m/z=722.5 [M+H]⁺. ¹H NMR (500 MHz, DMSO-d6) δ 12.95 (s, 1H), 10.52 (s, 1H), 10.12 (s, 1H), 7.97 (s, 1H), 7.59 (s, 1H), 7.48 (d, J=7.3 Hz, 1H), 7.36 (d, J=7.5 Hz, 2H), 4.42 (s, 1H), 3.93 (d, J=6.8 Hz, 2H), 3.75 (t, J=6.8 Hz, 2H), 3.58 (s, 1H), 3.29 (s, 2H), 3.08-2.99 (m, 4H), 2.84 (s, 1H), 2.79 (d, J=11.9 Hz, 3H), 2.72 (t, J=7.0 Hz, 3H), 2.13-2.07 (m, 2H), 1.91 (s, OH), 1.78 (d, J=12.2 Hz, 8H), 1.63 (s, 1H), 1.43 (d, J=12.5 Hz, 2H), 1.35 (s, 3H), 1.25-1.14 (m, 3H), 1.04 (d, J=12.7 Hz, 2H).

### Example 332. 1-(4-((4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)methyl)-2-fluorophenyl)dihydropyrimidine-2,4(1H,3H)-dione (205)

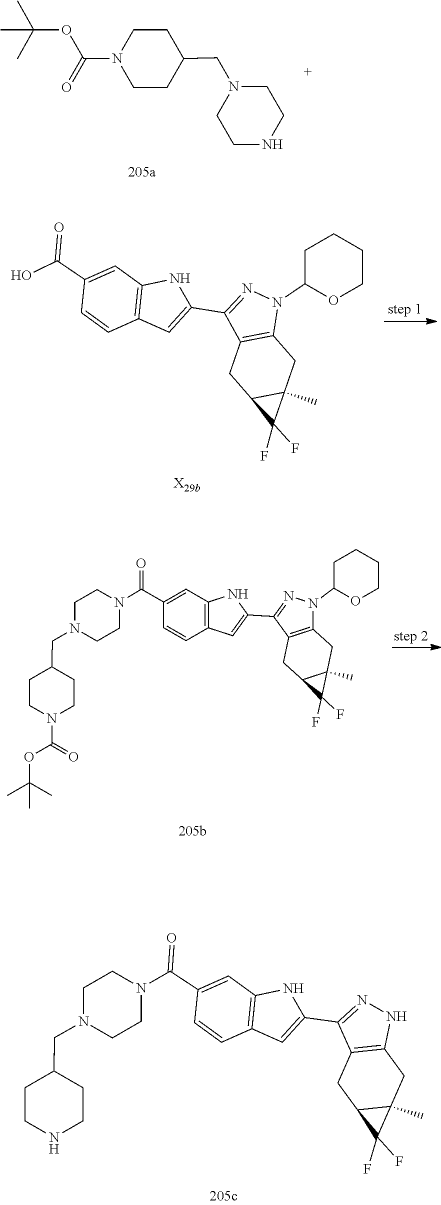 [SMILES: CC(C)(C)OC(=O)N1CCC(CN2CCNCC2)CC1 ; C[C@@]12Cc3c(c(-c4cc5ccc(C(=O)O)cc5[nH]4)nn3C3CCCCO3)C[C@@H]1C2(F)F ; CC(C)(C)OC(=O)N1CCC(CN2CCN(C(=O)c3ccc4cc(-c5nn(C6CCCCO6)c6c5C[C@@H]5C(F)(F)[C@]5(C)C6)[nH]c4c3)CC2)CC1 ; C[C@@]12Cc3[nH]nc(-c4cc5ccc(C(=O)N6CCN(CC7CCNCC7)CC6)cc5[nH]4)c3C[C@@H]1C2(F)F]

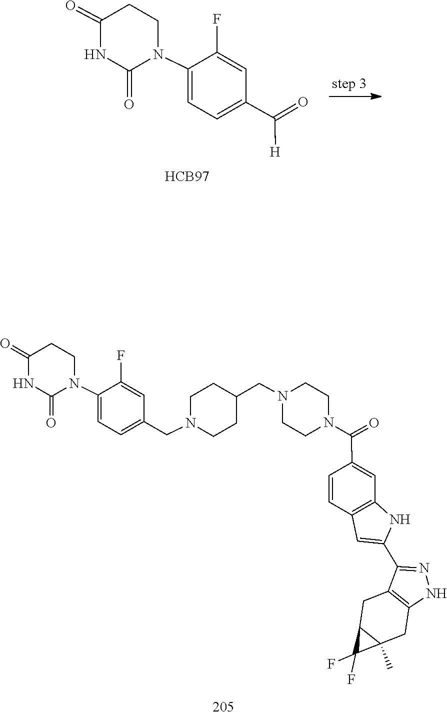 [SMILES: "HCB97" ; C[C@@]12Cc3[nH]nc(-c4cc5ccc(C(=O)N6CCN(CC7CCN(Cc8ccc(N9CCC(=O)NC9=O)c(F)c8)CC7)CC6)cc5[nH]4)c3C[C@@H]1C2(F)F ; O=Cc1ccc(N2CCC(=O)NC2=O)c(F)c1]

Step 1: to a solution of 2-[(4aS,5aR)-5,5-difluoro-5a-methyl-1-(oxan-2-yl)-4H,4aH,6H-cyclopropa[f]indazol-3-yl]-1H-indole-6-carboxylic acid (X₂₉b) (200.00 mg, 0.4679 mmol) and [(dimethylamino)({[1,2,3]triazolo[4,5-b]pyridin-3-yloxy})methylidene]dimethylazanium; hexafluoro-lambda5-phosphanuide (213.49 mg, 0.5615 mmol) in 2 ml DMF, N,N-diisopropylethylamine (490.32 uL, 362.84 mg, 2.8073 mmol) was added, stirred for 3 minutes, tert-butyl 4-(piperazin-1-ylmethyl)piperidine-1-carboxylate (205a) (132.61 mg, 0.4679 mmol) in 1 ml DMF was added, the reaction mixture was stirred for 1 hour. The reaction mixture was quenched by 1 ml water, the aqueous solution was extracted by DCM for three times. The combined organic solution was dried over Na₂SO₄ and concentrated. The crude product was purified by ISCO silica gel chromatography eluted with EtOAc in hexane (30%-100%) to afford tert-butyl 4-[(4-{2-[(4aS,5aR)-5,5-difluoro-5a-methyl-1-(oxan-2-yl)-4H,4aH,6H-cyclopropa[f]indazol-3-yl]-1H-indole-6-carbonyl}piperazin-1-yl)methyl]piperidine-1-carboxylate (301 mg, 92.9%)

Step 2: tert-butyl 4-[(4-{2-[(4aS,5aR)-5,5-difluoro-5a-methyl-1-(oxan-2-yl)-4H,4aH,6H-cyclopropa[f]indazol-3-yl]-1H-indole-6-carbonyl}piperazin-1-yl)methyl]piperidine-1-carboxylate (205b) (300.00 mg, 0.43 mmol) was dissolved in 5 ml hexafluoroisopropanol, 4M hydrogen chloride in dioxane (3.00 mL, 0.44 g, 12.00 mmol) was added, after stirring at rt for 30 minutes, the solution was evaporated under reduced pressure. The product was lyophilized to afford 2-[(4aS,5aR)-5,5-difluoro-5a-methyl-1H,4H,4aH,6H-cyclopropa[f]indazol-3-yl]-6-[4-(piperidin-4-ylmethyl)piperazine-1-carbonyl]-1H-indole HCL salt as fine white powder. Quantitative yield.

Step 3: To a solution of 4-(2,4-dioxo-1,3-diazinan-1-yl)-3-fluorobenzaldehyde (HCB97) (10.00 mg, 0.04 mmol) and 2-[(4aS,5aR)-5,5-difluoro-5a-methyl-1H,4H,4aH,6H-cyclopropa[f]indazol-3-yl]-6-[4-(piperidin-4-ylmethyl)piperazine-1-carbonyl]-1H-indole (205c) (21.53 mg, 0.04 mmol) in DCM (3 ml) and DMSO (1 ml), triethylamine (23.5 ul, 0.17 mmol) was added, sodium triacetoxyborohydride (13.46 mg, 0.06 mmol) was added in portion. The reaction mixture was stirred for 30 minutes. The reaction mixture was poured into 1 ml sat. NaHCO₃ solution, the solution was extracted by DCM twice. The organic layer was dried over Na₂SO₄ and concentrated. The crude product was purified by ISCO silica gel chromatography eluted with MeOH/DCM (5-20%) to afford 1-[4-({4-[(4-{2-[(4aS,5aR)-5,5-difluoro-5a-methyl-1H,4H,4aH,6H-cyclopropa[f]indazol-3-yl]-1H-indole-6-carbonyl}piperazin-1-yl)methyl]piperidin-1-yl}methyl)-2-fluorophenyl]-1,3-diazinane-2,4-dione (205) (5.8 mg, 18.8%). LCMS C₃₉H₄₃F₃N₈O₃ requires: 728.3 found: m/z=729.6 [M+H]⁺. ¹H NMR (500 MHz, DMSO-d6) δ 12.74 (s, 1H), 11.53 (d, J=44.2 Hz, 1H), 10.56 (s, 1H), 7.54 (t, J=43.9 Hz, 6H), 7.05 (d, J=37.8 Hz, 1H), 6.59 (d, J=57.0 Hz, 1H), 4.30 (s, 2H), 3.74 (d, J=7.0 Hz, 3H), 3.54 (s, 4H), 3.08 (qt, J=13.5, 8.8, 7.8 Hz, 8H), 2.87 (t, J=18.7 Hz, 3H), 2.74 (t, J=6.8 Hz, 2H), 2.38 (s, 2H), 2.12 (d, J=58.0 Hz, 1H), 1.90 (d, J=17.2 Hz, 2H), 1.40 (s, 3H), 1.20 (td, J=7.4, 2.4 Hz, 8H).

### Example 333. 1-(5-((4-((4-(2-((4aS,5aR)-5,5-difluoro-5a-methyl-1,4,4a,5,5a,6-hexahydrocyclopropa[f]indazol-3-yl)-1H-indole-6-carbonyl)piperazin-1-yl)methyl)piperidin-1-yl)methyl)-2-fluorophenyl)dihydropyrimidine-2,4(1H,3H)-dione (206)

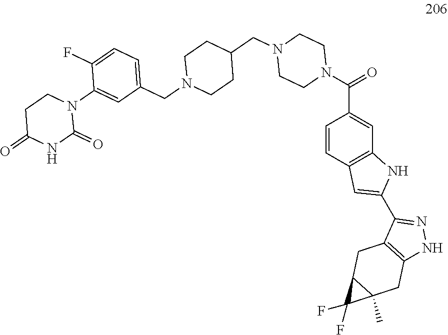 [SMILES: C[C@@]12Cc3[nH]nc(-c4cc5ccc(C(=O)N6CCN(CC7CCN(Cc8ccc(F)c(N9CCC(=O)NC9=O)c8)CC7)CC6)cc5[nH]4)c3C[C@@H]1C2(F)F]

This compound was synthesized following the same procedure as Compound 205. LCMS C₃₉H₄₃F₃N₈O₃ requires: 728.3 found: m/z=729.6 [M+H]⁺. ¹H NMR (500 MHz, DMSO) δ 12.71 (s, 1H), 11.46 (s, 1H), 10.47 (s, 1H), 7.55 (d, J=8.0 Hz, 1H), 7.44 (s, 1H), 7.34 (d, J=7.6 Hz, 1H), 7.30-7.20 (m, 2H), 7.00 (d, J=8.1 Hz, 1H), 6.64 (s, 1H), 3.72 (q, J=5.9 Hz, 2H), 3.18-2.94 (m, 3H), 2.82 (dd, J=25.4, 13.7 Hz, 3H), 2.73 (dt, J=8.4, 4.0 Hz, 2H), 2.56 (d, J=7.8 Hz, 9H), 2.36 (s, 4H), 2.16 (d, J=7.0 Hz, 2H), 2.01-1.82 (m, 6H), 1.68 (d, J=12.6 Hz, 2H), 1.51 (s, 1H), 1.40 (s, 3H), 1.12 (q, J=12.0 Hz, 2H), 0.99 (td, J=7.2, 2.5 Hz, 13H).

### Example 334. (4aS,5aR)-N-(1-(1-((1-(4-(2,4-dioxotetrahydropyrimidin-1(2H)-yl)phenyl)piperidin-4-yl)methyl)piperidin-4-yl)-1H-pyrazol-4-yl)-5,5-difluoro-5a-methyl-1,4,4a,5,5a,6-hexahydrocyclopropa[f]indazole-3-carboxamide (207)

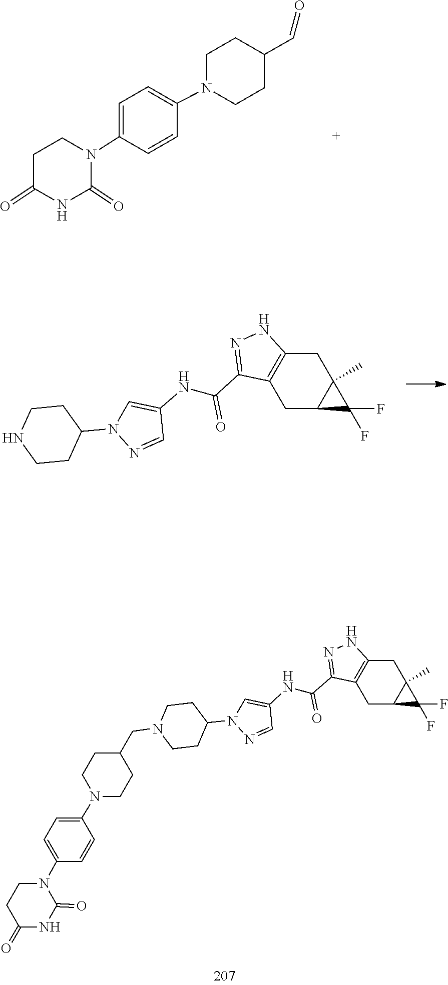 [SMILES: O=CC1CCN(c2ccc(N3CCC(=O)NC3=O)cc2)CC1 ; C[C@@]12Cc3[nH]nc(C(=O)Nc4cnn(C5CCNCC5)c4)c3C[C@@H]1C2(F)F ; C[C@@]12Cc3[nH]nc(C(=O)Nc4cnn(C5CCN(CC6CCN(c7ccc(N8CCC(=O)NC8=O)cc7)CC6)CC5)c4)c3C[C@@H]1C2(F)F]

To a 2 dram vial was added 1-[4-(2,4-dioxo-1,3-diazinan-1-yl)phenyl]piperidine-4-carbaldehyde (12.6 mg, 0.04 mmol), (4aS,5aR)-5,5-difluoro-5a-methyl-N-[1-(piperidin-4-yl)pyrazol-4-yl]-1H,4H,4aH,6H-cyclopropa[f]indazole-3-carboxamide hydrochloride (17.3 mg, 0.04 mmol), triethylamine (0.06 mL, 0.04 g, 0.42 mmol), DMSO (0.25 mL), and DCM (0.75 mL). To the reaction mixture was added sodium triacetoxyborohydride (30.80 mg, 0.15 mmol). After stirring the reaction mixture for 16 h, the reaction mixture was quenched with sat. NaHCO₃(aq), and the product was extracted with DCM (3×), dried over MgSO4, then concentrated. The resulting residue was purified by FC (4 g silica, 0-20% MeOH/DCM) to yield the title compound as a white solid (9.4 mg, 32%). LCMS: C₃₄H₄₁F₂N₉O₃ requires: 661.3, found: m/z=662.5 [M+H]⁺. ¹H NMR (500 MHz, DMSO-d6) δ 12.96 (s, 1H), 10.25 (s, 1H), 10.10 (s, 1H), 8.00 (s, 1H), 7.63 (s, 1H), 7.13 (d, J=8.2 Hz, 2H), 6.92 (d, J=8.4 Hz, 2H), 4.08 (s, 1H), 3.68 (d, J=9.7 Hz, 4H), 3.29 (d, J=2.5 Hz, 1H), 3.09-3.00 (m, 4H), 2.92 (d, J=10.9 Hz, 3H), 2.82 (d, J=17.1 Hz, 1H), 2.67 (t, J=7.8 Hz, 4H), 2.63 (s, 1H), 2.20 (d, J=7.1 Hz, 2H), 2.05 (t, J=11.5 Hz, 2H), 1.95 (s, 3H), 1.90 (d, J=11.6 Hz, 1H), 1.80 (d, J=11.8 Hz, 3H), 1.67 (s, 1H), 1.35 (s, 3H), 1.21 (d, J=15.5 Hz, 3H).

### Example 335. 1-(4-(4-((4-(2-(6,6-dimethyl-4,5,6,7-tetrahydro-1H-indazol-3-yl)-1H-indole-5-carbonyl)piperazin-1-yl)methyl)piperidin-1-yl)phenyl)dihydropyrimidine-2,4(1H,3H)-dione (208)

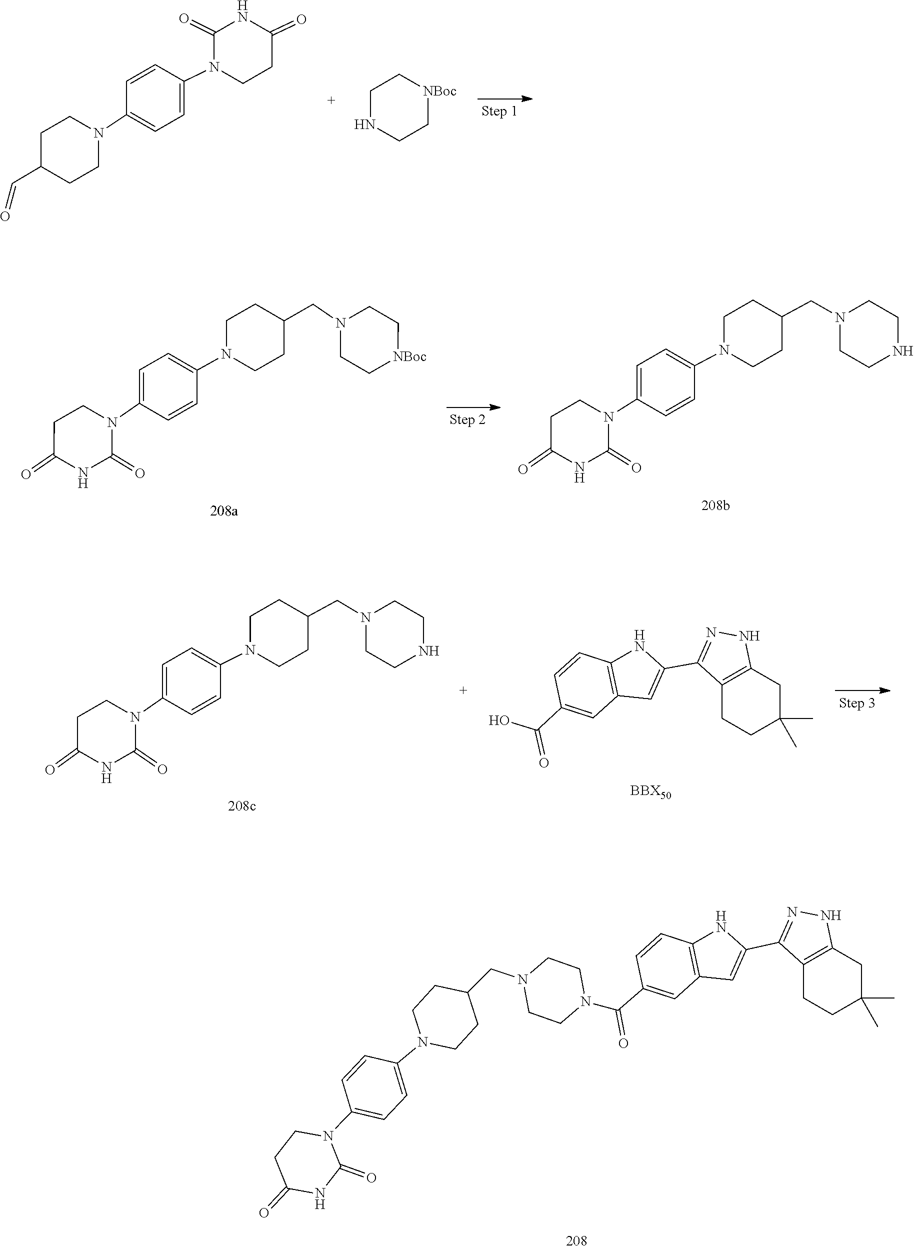

### Step 1: Synthesis of tert-butyl 4-((1-(4-(2,4-dioxotetrahydropyrimidin-1(2H)-yl)phenyl)piperidin-4-yl)methyl)piperazine-1-carboxylate

To a 20 mL vial was added 1-[4-(2,4-dioxo-1,3-diazinan-1-yl)phenyl]piperidine-4-carbaldehyde (13.00 mg, 0.04 mmol), tert-butyl piperazine-1-carboxylate (8.03 mg, 0.04 mmol), triethylamine (0.06 mL, 0.04 g, 0.43 mmol), DCM (0.75 mL), and DMSO (0.25 mL). To the reaction mixture was added sodium triacetoxyborohydride (27.43 mg, 0.13 mmol). The reaction mixture was stirred for 2 h, then quenched with sat. NaHCO₃ (aq). The product was extracted with DCM (3×), dried over Na₂SO₄, then concentrated. The resulting residue was purified by column chromatography (4 g silica, 0-10% MeOH/DCM) to yield the title compound as an off-white solid (10.1 mg, 50%). LCMS: C₂₅H₃₇N₅O₄ requires: 471.3, found: m/z=472.5 [M+H]⁺.

### Step 2: Synthesis of 1-(4-(4-(piperazin-1-ylmethyl)piperidin-1-yl)phenyl)dihydropyrimidine-2,4(1H,3H)-dione

To a 20 mL vial was added tert-butyl 4-({1-[4-(2,4-dioxo-1,3-diazinan-1-yl)phenyl]piperidin-4-yl}methyl)piperazine-1-carboxylate (10.10 mg, 0.02 mmol) and DCM (1.00 mL). To the reaction mixture was added 4N hydrogen chloride in dioxane (0.08 mL, 0.01 g, 0.32 mmol). After stirring overnight, the reaction mixture was concentrated to yield the title compound as a white solid (3.4 mg, 43%). LCMS: C₂₀H₂₉N₅O₂ requires: 371.3, found: m/z=372.5 [M+H]⁺.

### Step 3: Synthesis of 1-(4-(4-((4-(2-(6,6-dimethyl-4,5,6,7-tetrahydro-1H-indazol-3-yl)-1H-indole-5-carbonyl)piperazin-1-yl)methyl)piperidin-1-yl)phenyl)dihydropyrimidine-2,4(1H,3H)-dione

To a 1 dram vial was added 1-{4-[4-(piperazin-1-ylmethyl)piperidin-1-yl]phenyl}-1,3-diazinane-2,4-dione (3.4 mg, 0.01 mmol), 2-(6,6-dimethyl-1,4,5,7-tetrahydroindazol-3-yl)-1H-indole-5-carboxylic acid (2.8 mg, 0.01 mmol), N,N-diisopropylethylamine (0.02 mL, 0.01 g, 0.1 mmol), and DMF (1.0 mL). To the reaction mixture was added [(dimethylamino)({[1,2,3]triazolo[4,5-b]pyridin-3-yloxy})methylidene]dimethylazanium; hexafluoro-lambda5-phosphanuide (3.5 mg, 0.01 mmol). The reaction mixture was stirred for 1 h, then quenched with sat. NaHCO₃ (aq). The product was extracted with DCM (3×), dried over MgSO4, then concentrated. The resulting residue was purified by FC (4 g silica, 0-20% MeOH/DCM) to yield the title compound as a white solid (2.4 mg, 40%). LCMS: C₃₈H₄₆N₈O₃ requires: 662.4, found: m/z=663.6 [M+H]⁺.

### Example 336. (4aS,5aR)-N-(1-(1-((1-(4-(2,4-dioxotetrahydropyrimidin-1(2H)-yl)phenyl)pyrrolidin-3-yl)methyl)piperidin-4-yl)-1H-pyrazol-4-yl)-5,5-difluoro-5a-methyl-1,4,4a,5,5a,6-hexahydrocyclopropa[f]indazole-3-carboxamide (209)

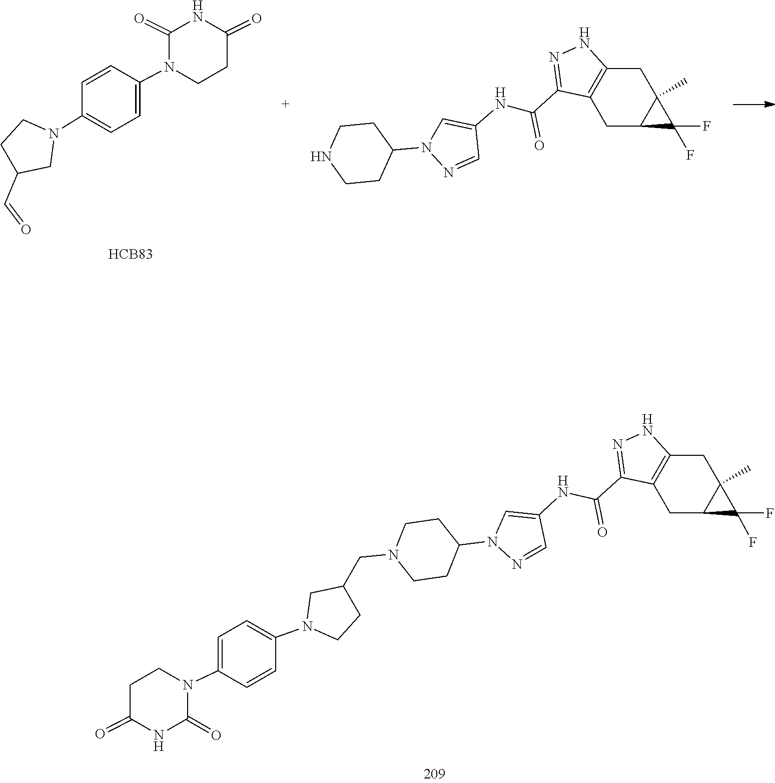 [SMILES: "HCB83" ; O=CC1CCN(c2ccc(N3CCC(=O)NC3=O)cc2)C1 ; C[C@@]12Cc3[nH]nc(C(=O)Nc4cnn(C5CCN(CC6CCN(c7ccc(N8CCC(=O)NC8=O)cc7)C6)CC5)c4)c3C[C@@H]1C2(F)F ; C[C@@]12Cc3[nH]nc(C(=O)Nc4cnn(C5CCNCC5)c4)c3C[C@@H]1C2(F)F]

To a 2 dram vial was added rac-(3R)-1-[4-(2,4-dioxo-1,3-diazinan-1-yl)phenyl]pyrrolidine-3-carbaldehyde (HCB83) (15.0 mg, 0.05 mmol), (4aS,5aR)-5,5-difluoro-5a-methyl-N-[1-(piperidin-4-yl)pyrazol-4-yl]-1H,4H,4aH,6H-cyclopropa[f]indazole-3-carboxamide hydrochloride (21.6 mg, 0.05 mmol), triethylamine (0.07 mL, 0.05 g, 0.52 mmol), DMSO (0.75 mL), and DCM (0.25 mL). To the reaction mixture was added sodium triacetoxyborohydride (30.80 mg, 0.15 mmol). After stirring the reaction mixture for 16 h, the reaction mixture was quenched with sat. NaHCO₃(aq), and the product was extracted with DCM (3×), dried over MgSO₄, then concentrated. The resulting residue was purified by FC (4 g silica, 0-20% MeOH/DCM) to yield the title compound as a white solid (19.6 mg, 55%). LCMS: C₃₃H₃₉F₂N₉O₃ requires: 647.3, found: m/z=648.5 [M+H]⁺. ¹H NMR (500 MHz, DMSO) δ 12.95 (s, 1H), 10.20 (s, 1H), 10.11 (s, 1H), 8.01 (s, 1H), 7.63 (s, 1H), 7.09 (d, J=8.3 Hz, 2H), 6.51 (d, J=8.3 Hz, 2H), 4.09 (s, 1H), 3.66 (t, J=6.8 Hz, 2H), 3.38 (d, J=8.4 Hz, 1H), 3.23 (t, J=8.0 Hz, 1H), 3.06 (d, J=9.7 Hz, 2H), 3.03-2.93 (m, 4H), 2.82 (d, J=17.1 Hz, 1H), 2.67 (t, J=6.7 Hz, 2H), 2.37 (d, J=7.7 Hz, 2H), 2.16-2.04 (m, 3H), 1.95 (s, 4H), 1.91 (d, J=10.6 Hz, 1H), 1.76 (s, 1H), 1.71 (d, J=10.4 Hz, 1H), 1.35 (s, 3H).

### Example 337. 1-(4-(3-((4-(2-(6,6-dimethyl-4,5,6,7-tetrahydro-1H-indazol-3-yl)-1H-indole-5-carbonyl)piperazin-1-yl)methyl)pyrrolidin-1-yl)phenyl)dihydropyrimidine-2,4(1H,3H)-dione (210)

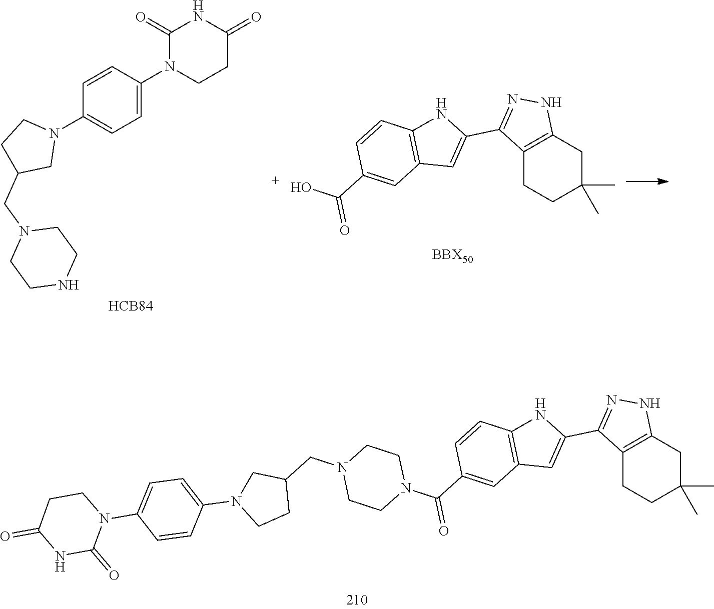

To a 1 dram vial was added rac-1-{4-[(3R)-3-(piperazin-1-ylmethyl)pyrrolidin-1-yl]phenyl}-1,3-diazinane-2,4-dione hydrochloride (HCB84) (17.0 mg, 0.04 mmol), 2-(6,6-dimethyl-1,4,5,7-tetrahydroindazol-3-yl)-1H-indole-5-carboxylic acid (BBX₅₀) (13.4 mg, 0.04 mmol), N,N-diisopropylethylamine (0.08 mL, 0.06 g, 0.43 mmol), and DMF (1.0 mL). To the reaction mixture was added [(dimethylamino)({[1,2,3]triazolo[4,5-b]pyridin-3-yloxy})methylidene]dimethylazanium; hexafluoro-lambda5-phosphanuide (16.4 mg, 0.04 mmol). The reaction mixture was stirred for 1 h, then quenched with sat. NaHCO₃(aq). The product was extracted with DCM (3×), dried over MgSO4, then concentrated. The resulting residue was purified by FC (4 g silica, 0-10% MeOH/DCM) to yield the title compound as a white solid (4.9 mg, 17%). LCMS: C₃₇H₄₄N₈O₃ requires: 648.4, found: m/z=649.5 [M+H]⁺. ¹H NMR (500 MHz, DMSO) δ 11.52 (s, 1H), 10.22 (s, 1H), 9.57 (s, 1H), 7.68 (s, 1H), 7.45 (d, J=8.3 Hz, 1H), 7.20 (d, J=8.2 Hz, 1H), 7.12 (d, J=8.5 Hz, 2H), 6.65 (s, 1H), 6.54 (d, J=8.5 Hz, 2H), 4.27 (s, 2H), 3.66 (t, J=6.7 Hz, 2H), 3.33 (s, 1H), 3.25 (q, J=8.1 Hz, 1H), 3.17 (s, 2H), 3.04 (t, J=8.5 Hz, 1H), 2.78 (d, J=9.0 Hz, 1H), 2.68 (t, J=6.6 Hz, 4H), 2.42 (s, 2H), 2.23 (s, 1H), 1.79 (s, 1H), 1.58 (t, J=6.3 Hz, 2H), 1.01 (s, 6H).

### Example 338. 1-(4-(3-((4-(2-(6,6-dimethyl-4,5,6,7-tetrahydro-1H-indazol-3-yl)-1H-indole-6-carbonyl)piperazin-1-yl)methyl)pyrrolidin-1-yl)phenyl)dihydropyrimidine-2,4(1H,3H)-dione (211)

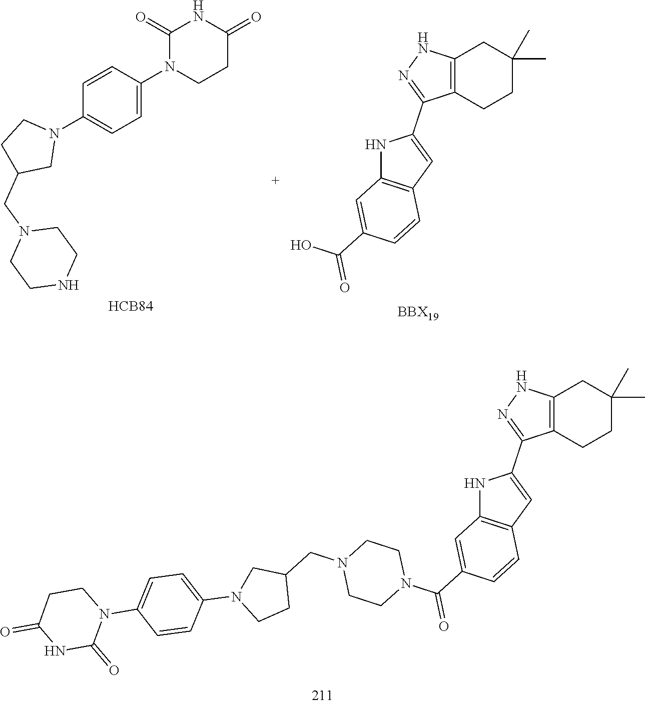 [SMILES: "HCB84" ; O=C1CCN(c2ccc(N3CCC(CN4CCNCC4)C3)cc2)C(=O)N1 ; CC1(C)CCc2c(-c3cc4ccc(C(=O)N5CCN(CC6CCN(c7ccc(N8CCC(=O)NC8=O)cc7)C6)CC5)cc4[nH]3)n[nH]c2C1 ; "+" ; CC1(C)CCc2c(-c3cc4ccc(C(=O)O)cc4[nH]3)n[nH]c2C1]

To a 1 dram vial was added rac-1-{4-[(3R)-3-(piperazin-1-ylmethyl)pyrrolidin-1-yl]phenyl}-1,3-diazinane-2,4-dione hydrochloride (HCB84) (17.0 mg, 0.04 mmol), 2-(6,6-dimethyl-1,4,5,7-tetrahydroindazol-3-yl)-1H-indole-6-carboxylic acid (BBX₁₉) (13.4 mg, 0.04 mmol), N,N-diisopropylethylamine (0.08 mL, 0.06 g, 0.43 mmol), and DMF (1.0 mL). To the reaction mixture was added [(dimethylamino)({[1,2,3]triazolo[4,5-b]pyridin-3-yloxy})methylidene]dimethylazanium; hexafluoro-lambda5-phosphanuide (13.7 mg, 0.04 mmol). The reaction mixture was stirred for 1 h, then quenched with sat. NaHCO₃(aq). The product was extracted with DCM (3×), dried over MgSO4, then concentrated. The resulting residue was purified by FC (4 g silica, 0-10% MeOH/DCM) to yield the title compound as a white solid (6.7 mg, 26%). LCMS: C₃₇H₄₄N₈O₃ requires: 648.4, found: m/z=649.5 [M+H]⁺. ¹H NMR (500 MHz, DMSO) δ 11.53 (s, 1H), 10.22 (s, 1H), 9.60 (s, 1H), 7.60-7.52 (m, 2H), 7.11 (dd, J=11.2, 8.0 Hz, 3H), 6.64 (s, 1H), 6.54 (d, J=8.5 Hz, 2H), 4.26 (s, 2H), 3.66 (t, J=6.7 Hz, 2H), 3.52 (t, J=8.5 Hz, 1H), 3.39-3.32 (m, 1H), 3.33 (s, 1H), 3.25 (q, J=8.2 Hz, 1H), 3.16 (s, 2H), 3.03 (t, J=8.5 Hz, 1H), 2.80-2.74 (m, 1H), 2.68 (t, J=6.5 Hz, 4H), 2.42 (s, 2H), 2.23 (d, J=11.2 Hz, 1H), 1.78 (dd, J=12.2, 8.5 Hz, 1H), 1.58 (t, J=6.4 Hz, 2H), 1.01 (s, 6H).

### Example 339. (4aS,5aR)-N-[1-(1-{1-[4-(2,4-Dioxo-1,3-diazinan-1-yl)phenyl]piperidine-4-carbonyl}piperidin-4-yl)pyrazol-4-yl]-5,5-difluoro-5a-methyl-1H,4H,4aH,6H-cyclopropa[f]indazole-3-carboxamide (212)

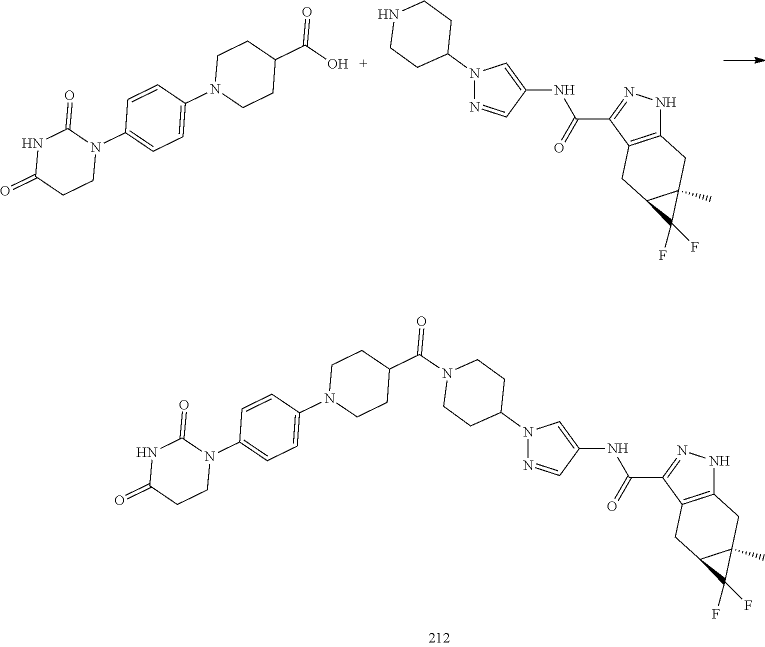

To a solution of 1-[4-(2,4-dioxo-1,3-diazinan-1-yl)phenyl]piperidine-4-carboxylic acid (4.20 mg, 0.01 mmol) and (4aS,5aR)-5,5-difluoro-5a-methyl-N-[1-(piperidin-4-yl)pyrazol-4-yl]-1H,4H,4aH,6H-cyclopropa[f]indazole-3-carboxamide (4.98 mg, 0.01 mmol) in 0.2 ml DMF, N,N-diisopropylethylamine (18.5 ul, 0.11 mmol) was added, [(dimethylamino)({[1,2,3]triazolo[4,5-b]pyridin-3-yloxy})methylidene]dimethylazanium; hexafluoro-lambda5-phosphanuide (5.03 mg, 0.01 mmol) in 0.2 ml DMF was added. The reaction mixture was stirred for 30 minutes. The reaction mixture was poured into water, extracted by DCM twice. The organic solution was dried over Na₂SO₄ and concentrated. The crude product was purified by ISCO silica gel chromatography eluted with MeOH/DCM (4-20%) to afford (4aS,5aR)-N-[1-(1-{1-[4-(2,4-dioxo-1,3-diazinan-1-yl)phenyl]piperidine-4-carbonyl}piperidin-4-yl)pyrazol-4-yl]-5,5-difluoro-5a-methyl-1H,4H,4aH,6H-cyclopropa[f]indazole-3-carboxamide (3.4 mg, 36%). LCMS C₃₄H₃₉F₂N₉O₄ required: 675.3 found: m/z=676.5 [M+H]+¹H NMR (500 MHz, DMSO-d6) δ 12.97 (s, 1H), 10.27 (s, 1H), 10.14 (s, 1H), 8.06 (s, 1H), 7.65 (s, 1H), 7.16 (d, J=8.3 Hz, 2H), 6.96 (d, J=8.4 Hz, 2H), 4.60-4.47 (m, 1H), 4.40 (d, J=12.3 Hz, 1H), 4.13 (d, J=13.5 Hz, 1H), 3.72 (q, J=8.3, 7.8 Hz, 4H), 3.64 (t, J=6.7 Hz, 1H), 3.19-2.99 (m, 4H), 2.96-2.60 (m, 7H), 2.05 (dd, J=31.6, 12.1 Hz, 2H), 1.96-1.61 (m, 8H), 1.37 (s, 3H), 1.26 (dq, J=10.0, 4.4, 2.7 Hz, 8H).

### Example 340. 1-(6-{4-[(4-{2-[(4aS,5aR)-5,5-difluoro-5a-methyl-1H,4H,4aH,6H-cyclopropa[f]indazol-3-yl]-1H-indole-6-carbonyl}piperazin-1-yl)methyl]piperidin-1-yl}pyridin-3-yl)-1,3-diazinane-2,4-dione (213)

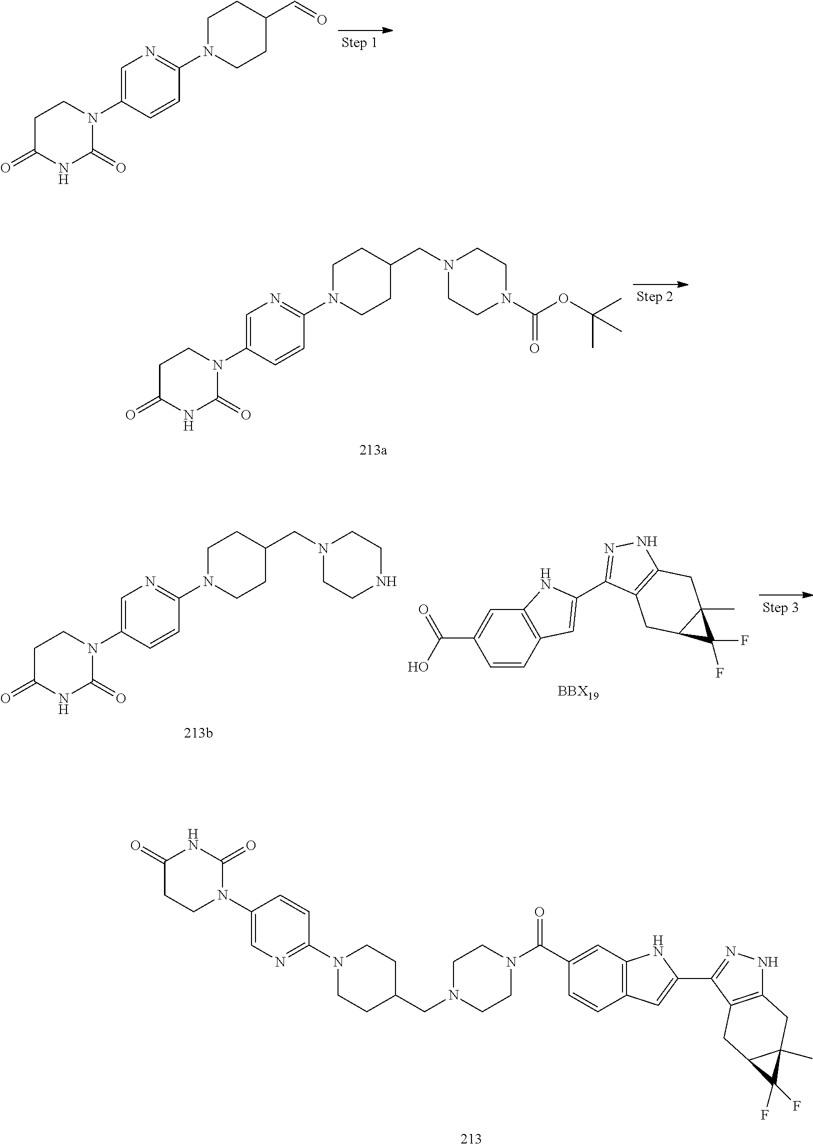 [SMILES: O=CC1CCN(c2ccc(N3CCC(=O)NC3=O)cn2)CC1 ; CC(C)(C)OC(=O)N1CCN(CC2CCN(c3ccc(N4CCC(=O)NC4=O)cn3)CC2)CC1 ; O=C1CCN(c2ccc(N3CCC(CN4CCNCC4)CC3)nc2)C(=O)N1 ; C[C@@]12Cc3[nH]nc(-c4cc5ccc(C(=O)O)cc5[nH]4)c3C[C@@H]1C2(F)F ; C[C@@]12Cc3[nH]nc(-c4cc5ccc(C(=O)N6CCN(CC7CCN(c8ccc(N9CCC(=O)NC9=O)cn8)CC7)CC6)cc5[nH]4)c3C[C@@H]1C2(F)F]

Step 1: To a solution of 1-[5-(2,4-dioxo-1,3-diazinan-1-yl)pyridin-2-yl]piperidine-4-carbaldehyde (40.30 mg, 0.13 mmol), tert-butyl piperazine-1-carboxylate (24.83 mg, 0.13 mmol) in 0.25 ml DMSO and 1 ml DCM, N,N-diisopropylethylamine (17.2 mg, 0.13 mmol) was added, sodium triacetoxyborohydride (56.50 mg, 0.27 mmol) was added in portion, the reaction was stirred for 20 minutes, LCMS indicated reaction is completed. The reaction mixture was quenched by 1 ml water, the aqueous layer was extracted by DCM for three times. The combined organic layer was dried over Na₂SO₄ and concentrated. The crude product was purified by ISCO silica gel chromatography eluted with methanol in DCM (0-10%) to afford tert-butyl 4-({1-[5-(2,4-dioxo-1,3-diazinan-1-yl)pyridin-2-yl]piperidin-4-yl}methyl)piperazine-1-carboxylate (52 mg, 82.6%). LCMS: C₂₄H₃₆N₆O₄ requires: 472.3 found: m/z=473.3.

Step 2: tert-butyl 4-({1-[5-(2,4-dioxo-1,3-diazinan-1-yl)pyridin-2-yl]piperidin-4-yl}methyl)piperazine-1-carboxylate (52 mg, 0.11 mmol) was dissolved in 5% trifluoroacetic acid in hexafluoroisopropanol (2 ml), after stirring for 30 minutes, the solution was evaporated under reduced pressure. The product was lyophilized to afford 1-{6-[4-(piperazin-1-ylmethyl)piperidin-1-yl]pyridin-3-yl}-1,3-diazinane-2,4-dione TFA salt. Quantitative yield. LCMS: C₁₉H₂₈N₆O₂ requires: 372.2 found: m/z=373.3.

Step 3: To a solution of 1-{6-[4-(piperazin-1-ylmethyl)piperidin-1-yl]pyridin-3-yl}-1,3-diazinane-2,4-dione; bis(trifluoroacetaldehyde) (21.80 mg, 0.0383 mmol) and 2-[(4aS,5aR)-5,5-difluoro-5a-methyl-1H,4H,4aH,6H-cyclopropa[f]indazol-3-yl]-1H-indole-6-carboxylic acid (13.17 mg, 0.0383 mmol) in 2 ml DMF, N,N-diisopropylethylamine (40.18 uL, 29.74 mg, 0.2301 mmol) was added, [(dimethylamino)({[1,2,3]triazolo[4,5-b]pyridin-3-yloxy})methylidene]dimethylazanium; hexafluoro-lambda5-phosphanuide (17.50 mg, 0.0460 mmol) in 1 ml DMF was added, the reaction mixture was stirred for 1 hour. The reaction mixture was quenched by 2 ml water, the aqueous layer was extracted by DCM for three times. The combined organic layer was dried over Na₂SO₄ and concentrated. The crude product was purified by ISCO silica gel chromatography eluted with methanol in DCM (0-20%) to afford 1-(6-{4-[(4-{2-[(4aS,5aR)-5,5-difluoro-5a-methyl-1H,4H,4aH,6H-cyclopropa[f]indazol-3-yl]-1H-indole-6-carbonyl}piperazin-1-yl)methyl]piperidin-1-yl}pyridin-3-yl)-1,3-diazinane-2,4-dione (19 mg, 71% yield). LCMS C₃₇H₄₁F₂N₉O₃ requires: 697.3 found: [M+H+]=698.5 ¹H NMR (500 MHz, DMSO-d6) δ 12.74 (s, 1H), 11.54 (d, J=39.3 Hz, 1H), 10.35 (s, 1H), 9.60 (s, 1H), 8.06 (s, 1H), 7.52 (dd, J=34.0, 10.4 Hz, 3H), 7.07 (d, J=33.0 Hz, 1H), 6.87 (s, 1H), 6.66 (s, 1H), 4.29 (s, 3H), 3.70 (t, J=6.7 Hz, 2H), 3.66-3.45 (m, 2H), 3.21-2.96 (m, 5H), 2.85 (d, J=16.8 Hz, 3H), 2.71 (t, J=6.7 Hz, 2H), 2.39 (d, J=12.4 Hz, 1H), 2.15 (d, J=50.3 Hz, 1H), 1.94-1.72 (m, 3H), 1.40 (s, 3H), 1.31-1.02 (m, 4H).

### Example 341. 3-{2-[4-({1-[2-(6,6-dimethyl-1,4,5,7-tetrahydroindazol-3-yl)-1H-indole-6-carbonyl]piperidin-4-yl}methyl)piperazin-1-yl]pyridin-4-yl}piperidine-2,6-dione (214)

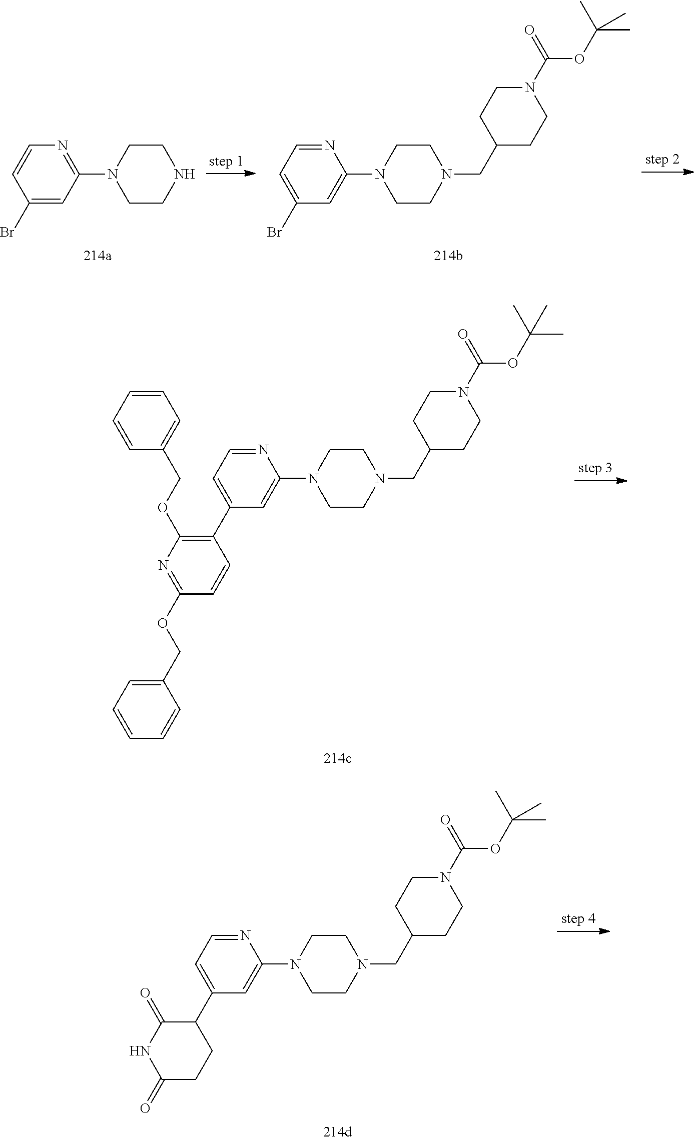 [SMILES: Brc1ccnc(N2CCNCC2)c1 ; CC(C)(C)OC(=O)N1CCC(CN2CCN(c3cc(Br)ccn3)CC2)CC1 ; CC(C)(C)OC(=O)N1CCC(CN2CCN(c3cc(-c4ccc(OCc5ccccc5)nc4OCc4ccccc4)ccn3)CC2)CC1 ; CC(C)(C)OC(=O)N1CCC(CN2CCN(c3cc(C4CCC(=O)NC4=O)ccn3)CC2)CC1]

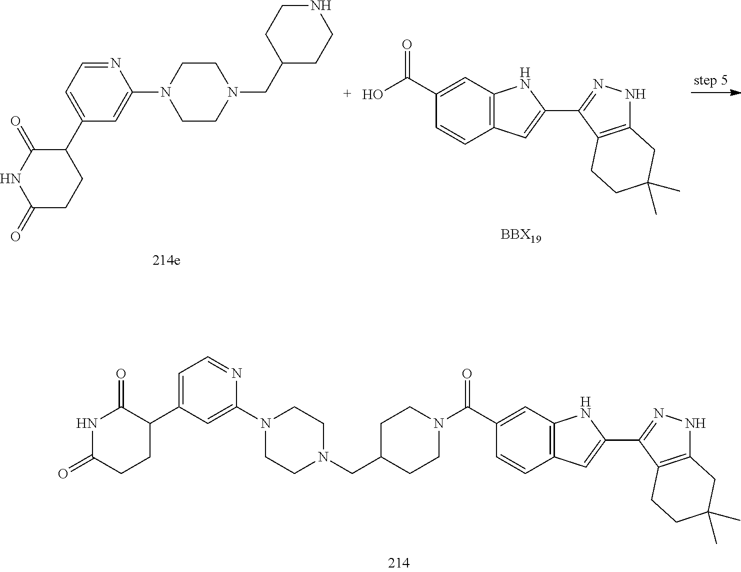

Step 1: To a solution of tert-butyl 4-formylpiperidine-1-carboxylate (220.22 mg, 1.03 mmol) and 1-(4-bromopyridin-2-yl)piperazine (250.00 mg, 1.03 mmol) in DCM, NaBH(OAc)3 (0.66 g, 3.097 mmol) was added, stirred for 0.5 h. Quenched the reaction mixture with 10% NaHCO₃ in water solution, extracted with CH₂Cl₂ twice. The organic solution was dried over sodium sulfate, filtered and concentrated. The crude product was purified by column chromatography on silica gel eluted with 20-100% EtOAc/Hexane to obtain tert-butyl 4-{[4-(4-bromopyridin-2-yl)piperazin-1-yl]methyl}piperidine-1-carboxylate (251 mg, 0.571 mmol, 55.3% yield) LCMS C₂₀H₃₁BrN₄O₂ required: 438.16, found: m/z=463.2 [M+Na]⁺.

Step 2: tert-butyl 4-{[4-(4-bromopyridin-2-yl)piperazin-1-yl]methyl}piperidine-1-carboxylate (310.00 mg, 0.71 mmol), 2,6-bis(benzyloxy)pyridin-3-ylboronic acid (236.47 mg, 0.71 mmol) and Pd(dppf)Cl₂-DCM (115.23 mg, 0.14 mmol) and cesium carbonate (689.61 mg, 2.12 mmol) were deposited in a microwave vial in dioxane (5.00 mL) and water (1.25 mL), bubbled nitrogen gas, the mixture was microwaved at 100° C. for 2.5 hours. The organic layer was loaded directly onto a silica gel cartridge and the mixture was purified by flash chromatography eluted with EtOAc/hexane (20-100%) to afford tert-butyl 4-({4-[2,6-bis(benzyloxy)-[3,4′-bipyridin]-2′-yl]piperazin-1-yl}methyl)piperidine-1-carboxylate (420 mg, 0.643 mmol, 91.6%). LCMS C₃₉H₄₇N₅O₄ required: 649.36, found: m/z=650.5 [M+H]⁺.

Step 3: tert-butyl 4-({4-[2,6-bis(benzyloxy)-[3,4′-bipyridin]-2′-yl]piperazin-1-yl}methyl)piperidine-1-carboxylate (200 mg, 0.308 mmol)_and 10% Pd/C (50 mg) were stirred in EtOH (3.00 mL) and THE (3.00 mL) under a balloon of H₂ for 16 hours. The mixture was diluted with THE and filtered through celite. The mixture was concentrated in vacuo and purified by flash chromatography on silica gel column (gradient elution with zero to 8% MeOH:DCM) to provide tert-butyl 4-({4-[4-(2,6-dioxopiperidin-3-yl)pyridin-2-yl]piperazin-1-yl}methyl)piperidine-1-carboxylate (0.083 g, 57.2%). LCMS: C₂₅H₃₇N₅O₄ requires 471.3, found: m/z=472.7 [M+H]⁺.

Step 4: To a solution of tert-butyl 4-({4-[4-(2,6-dioxopiperidin-3-yl)pyridin-2-yl]piperazin-1-yl}methyl)piperidine-1-carboxylate (83 mg, 0.176 mmol) in 1 mL DCM, 4 N HCl in dioxane (4 mL) was added, stirred for 20 minutes, The mixture was concentrated in vacuo to provide 3-{2-[4-(piperidin-4-ylmethyl)piperazin-1-yl]pyridin-4-yl}piperidine-2,6-dione (71.8 mg, 100%) as an HCl salt. LCMS: C₂₀H₂₉N₅O₂ requires: 371.2, found: m/z=372.4 [M+H]⁺.

Step 5: To a solution of 3-{2-[4-(piperidin-4-ylmethyl)piperazin-1-yl]pyridin-4-yl}piperidine-2,6-dione (40 mg, 0.108 mmol) and 2-(6,6-dimethyl-1,4,5,7-tetrahydroindazol-3-yl)-1H-indole-6-carboxylic acid (33.3 mg, 0.108 mmol) in 0.5 ml DMF, N,N-diisopropylethylamine (55.7 mg, 0.431 mmol) added, stirred for 3 minutes, HATU (40.9 mg, 0.108 mmol) in 0.2 ml DMF was added, the mixture was stirred for 20 minutes. The reaction mixture was quenched by 1 ml water, extracted by DCM×3. The organic layer was dried over Na₂SO$ and concentrated. Silica gel column purification eluted with MEOH/DCM (0-20%), obtained 3-{2-[4-({1-[2-(6,6-dimethyl-1,4,5,7-tetrahydroindazol-3-yl)-1H-indole-6-carbonyl]piperidin-4-yl}methyl)piperazin-1-yl]pyridin-4-yl}piperidine-2,6-dione (13 mg, 17.3%). LCMS: C₃₈H₄₆N₈O₃ requires: 662.3, found: m/z=663.5 [M+H]⁺. ¹H NMR (500 MHz, DMSO) δ 12.57 (s, 1H), 11.43 (s, 1H), 10.87 (s, 1H), 8.04 (d, J=5.1 Hz, 1H), 7.54 (d, J=8.1 Hz, 1H), 7.45 (s, 1H), 7.00 (d, J=8.1 Hz, 1H), 6.71 (s, 1H), 6.62 (s, 1H), 6.56-6.48 (m, 1H), 3.77 (dd, J=11.9, 5.0 Hz, 1H), 3.63 (q, J=6.2 Hz, 1H), 3.55-3.39 (m, 4H), 3.15 (dt, J=11.5, 5.7 Hz, 1H), 2.92 (s, 1H), 2.76-2.57 (m, 2H), 2.44 (d, J=11.8 Hz, 7H), 2.31-2.14 (m, 3H), 2.07-1.96 (m, 1H), 1.83 (d, J=49.4 Hz, 3H), 1.59 (t, J=6.3 Hz, 2H), 1.21-1.06 (m, 2H), 1.02 (s, 6H).

### Example 342. 3-(5-(4-((4-(2-(6,6-dimethyl-4,5,6,7-tetrahydro-1H-indazol-3-yl)-1H-indole-6-carbonyl)piperazin-1-yl)methyl)piperidin-1-yl)pyridin-2-yl)piperidine-2,6-dione (215)

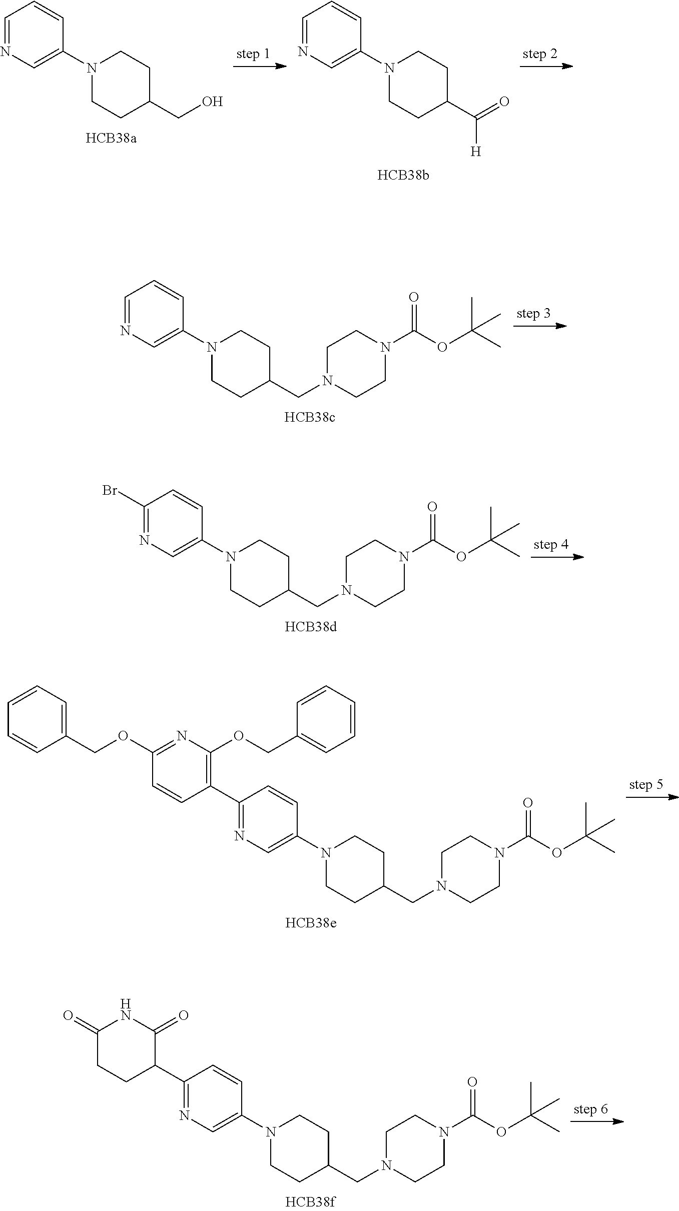 [SMILES: OCC1CCN(c2cccnc2)CC1 ; O=CC1CCN(c2cccnc2)CC1 ; CC(C)(C)OC(=O)N1CCN(CC2CCN(c3cccnc3)CC2)CC1 ; CC(C)(C)OC(=O)N1CCN(CC2CCN(c3ccc(Br)nc3)CC2)CC1 ; CC(C)(C)OC(=O)N1CCN(CC2CCN(c3ccc(-c4ccc(OCc5ccccc5)nc4OCc4ccccc4)nc3)CC2)CC1 ; CC(C)(C)OC(=O)N1CCN(CC2CCN(c3ccc(C4CCC(=O)NC4=O)nc3)CC2)CC1]

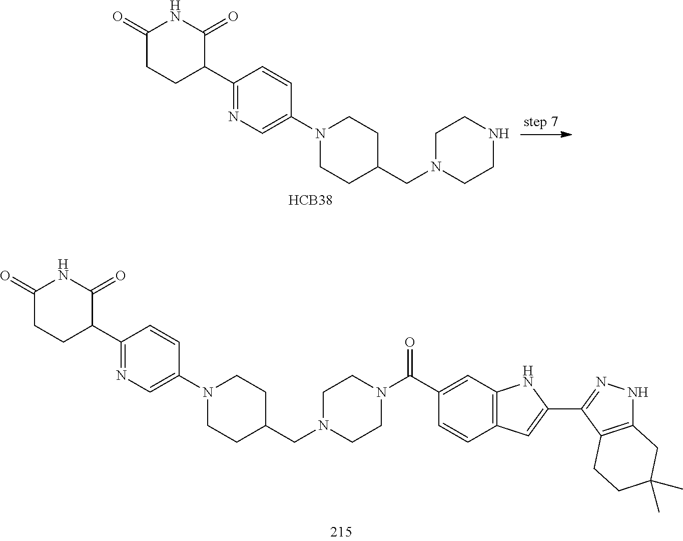

Step 1: To a solution of [1-(pyridin-3-yl)piperidin-4-yl]methanol (500.00 mg, 2.60 mmol) in DMSO, triethylamine (7.31 mL, 5.26 g, 52.01 mmol) was added, then sulfur trioxide pyridine complex (4.14 g, 26.01 mmol) was added, stirred for 30 minutes. TLC showed. Dissolved the crude product in EtOAc, the organic solution was washed by water twice. The organic layer was dried over Na₂SO₄ and concentrated to afford 1-(pyridin-3-yl)piperidine-4-carbaldehyde (0.4800 g, 97.0%). The crude product was used to the next step without purification.

Step 2: To a solution of 1-(pyridin-3-yl)piperidine-4-carbaldehyde (480.00 mg, 2.52 mmol) and tert-butyl piperazine-1-carboxylate (0.47 g, 2.52 mmol) in 10 mL DCM, NaBH(OAc)₃ (1/6 g, 7.57 mmol) was added, the mixture was stirred for 0.5 hr. The reaction mixture was quenched with 20% NaHCO₃ solution, the solution was extracted with DCM three times. The organic solution was dried over Na₂SO₄ and concentrated. Silica gel column purification eluted with EtOAc for 5 minutes, then eluted with 0-20% MeOH in EtOAc to afford tert-butyl 4-{[1-(pyridin-3-yl)piperidin-4-yl]methyl}piperazine-1-carboxylate (868 mg, 95.4%). H NMR (500 MHz, DMSO-d6) δ 8.29 (d, J=3.0 Hz, 1H), 7.95 (dd, J=4.6, 1.4 Hz, 1H), 7.30 (ddd, J=8.7, 3.0, 1.5 Hz, 1H), 7.19 (dd, J=8.5, 4.5 Hz, 1H), 3.74 (dt, J=12.6, 3.6 Hz, 2H), 3.31 (d, J=5.5 Hz, 4H), 2.69 (td, J=12.2, 2.6 Hz, 2H), 2.55 (s, 6H), 2.30 (t, J=5.1 Hz, 4H), 2.17 (d, J=7.3 Hz, 2H), 1.79 (d, J=13.0 Hz, 2H), 1.40 (s, 9H), 1.20 (qd, J=12.2, 4.0 Hz, 2H).

Step 3: To a solution of tert-butyl 4-{[1-(pyridin-3-yl)piperidin-4-yl]methyl}piperazine-1-carboxylate (868.00 mg, 2.41 mmol) in 20 ml acetonitrile, NBS (514.25 mg, 2.89 mmol) in 7 ml acetonitrile was added slowly, the mixture was stirred at 0° C. for 1 hr, Acetonitrile was evaporated, the crude product was purified by silica gel chromatography eluted with EtOAc in DCM (0-100%) to afford tert-butyl 4-{[1-(6-bromopyridin-3-yl)piperidin-4-yl]methyl}piperazine-1-carboxylate (451 mg, 42.6%). ¹H NMR (500 MHz, DMSO) δ 8.05 (d, J=3.2 Hz, 1H), 7.37 (d, J=8.8 Hz, 1H), 7.31 (dd, J=8.9, 3.2 Hz, 1H), 3.73 (d, J=12.7 Hz, 2H), 2.72 (td, J=12.3, 2.6 Hz, 2H), 2.29 (t, J=5.1 Hz, 4H), 2.16 (d, J=7.2 Hz, 2H), 1.78 (d, J=13.5 Hz, 2H), 1.71 (ddd, J=11.1, 7.3, 3.6 Hz, OH), 1.40 (s, 9H), 1.17 (qd, J=12.3, 4.0 Hz, 2H).

Step 4: tert-butyl 4-{[1-(pyridin-3-yl)piperidin-4-yl]methyl}piperazine-1-carboxylate (451 mg, 1.03 mmol) and Pd(dppf)C₁₂-DCM (167.6 mg, 0.20 mmol) and caesium carbonate (1.0 g, 3.08 mmol) were deposited in a microwave vial in dioxane (9.00 mL) and water (2.25 mL), bubbled nitrogen gas, the mixture was microwaved at 100° C. for 2 hours. The organic layer was loaded directly onto a silica gel cartridge and the mixture was purified by flash chromatography eluted with EtOAc/hexane (0-100%) to afford tert-butyl 4-({1-[2′,6′-bis(benzyloxy)-[2,3′-bipyridin]-5-yl]piperidin-4-yl}methyl)piperazine-1-carboxylate (620 mg, 0.954 mmol, 92.9%). LCMS: C₃₉H₄₇N₅O₄ required: 649.36, found: m/z=650.7 [M+H]⁺.

Step 5: tert-butyl 4-({1-[2′,6′-bis(benzyloxy)-[2,3′-bipyridin]-5-yl]piperidin-4-yl}methyl)piperazine-1-carboxylate (620 mg, 0.954 mmol) and 10% Pd/C (100 mg) were stirred in EtOH (3.00 mL) and THE (12.00 mL) under a balloon of H₂ for 24 hours. The mixture was diluted with THF and filtered through celite. The mixture was concentrated in vacuo and purified by flash chromatography on silica gel column (gradient elution with zero to 9% MeOH:DCM) to provide tert-butyl 4-[(1-{6-[(3R)-2,6-dioxopiperidin-3-yl]pyridin-3-yl}piperidin-4-yl)methyl]piperazine-1-carboxylate (0.124 g, 27.5%). LCMS: C₂₅H₃₇N₅O₄ requires 471.3, found: m/z=472.5 [M+H]⁺.

Step 6: To a solution of tert-butyl 4-[(1-{6-[(3R)-2,6-dioxopiperidin-3-yl]pyridin-3-yl}piperidin-4-yl)methyl]piperazine-1-carboxylate (0.123 g) in 1 mL DCM, 0.5 mL of TFA was added, the reaction was stirred for 40 minutes, The mixture was concentrated in vacuo to provide 3-{5-[4-(piperazin-1-ylmethyl)piperidin-1-yl]pyridin-2-yl}piperidine-2,6-dione (HCB38) (100 mg, 100%) as an TFA salt. LCMS: C₂₀H₂₉N₅O₂ requires: 371.2, found: m/z=372.3 [M+H]⁺.

Step 7: To a solution of 3-{5-[4-(piperazin-1-ylmethyl)piperidin-1-yl]pyridin-2-yl}piperidine-2,6-dione (HCB38) (25 mg, 0.0673 mmol) and 2-(6,6-dimethyl-1,4,5,7-tetrahydroindazol-3-yl)-1H-indole-6-carboxylic acid (20.8 mg, 0.0673 mmol) in 0.5 ml DMF, N,N-diisopropylethylamine (43.5 mg, 0.337 mmol) was added, the reaction mixture was stirred for 3 minutes, HATU (25.6 mg, 0.0673 mmol) in 0.2 ml DMF was added, the mixture was stirred for 20 minutes. The crude reaction mixture was purified by prep-HPLC eluted with 5-95% acetonitrile in water with 0.01% TFA to afford 3-{5-[4-(piperazin-1-ylmethyl)piperidin-1-yl]pyridin-2-yl}piperidine-2,6-dione (215) (15 mg, 32% yield). LCMS: C₃₈H₄₆N₈O₃ requires: 662.4, found: m/z=663.6 [M+H]⁺. ¹H NMR (500 MHz, CD₃CN) 6 10.02 (s, 1H), 8.77 (s, 1H), 8.22 (d, J=3.0 Hz, 1H), 7.64 (d, J=8.1 Hz, 1H), 7.57 (s, 1H), 7.30 (dd, J=8.7, 3.0 Hz, 1H), 7.21-7.11 (m, 2H), 6.74 (d, J=2.0 Hz, 1H), 3.89 (dd, J=8.9, 5.2 Hz, 1H), 3.74 (dt, J=13.0, 3.4 Hz, 2H), 3.68-3.49 (m, 1H), 2.93 (d, J=9.3 Hz, 2H), 2.83-2.71 (m, 4H), 2.69-2.53 (m, 2H), 2.48 (s, 2H), 2.43-2.11 (m, 3H), 1.95-1.90 (m, 2H), 1.66 (t, J=6.4 Hz, 2H), 1.39 (dt, J=12.1, 6.0 Hz, 2H), 1.30 (s, 1H), 1.24 (s, 1H), 1.06 (s, 6H).

### Example 343. 3-{5-[4-({4-[2-(6,6-dimethyl-1,4,5,7-tetrahydroindazol-3-yl)-5-fluoro-1H-indole-6-carbonyl]piperazin-1-yl}methyl)piperidin-1-yl]pyridin-2-yl}piperidine-2,6-dione (216)

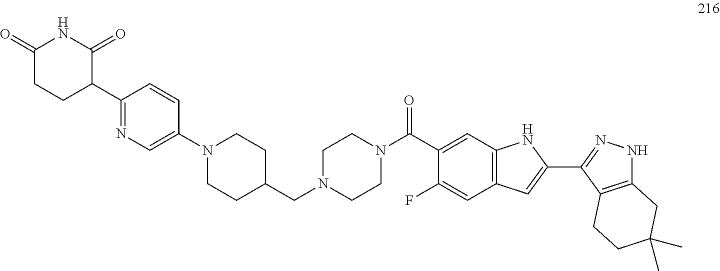 [SMILES: CC1(C)CCc2c(-c3cc4cc(F)c(C(=O)N5CCN(CC6CCN(c7ccc(C8CCC(=O)NC8=O)nc7)CC6)CC5)cc4[nH]3)n[nH]c2C1]

Followed the procedure described for Compound 215 above starting from 2-(6,6-dimethyl-4,5,6,7-tetrahydro-1H-indazol-3-yl)-5-fluoro-1H-indole-6-carboxylic acid. LCMS: C₃₈H₄₅FN₈O₃ requires: 680.4, found: m/z=681.7 [M+H]⁺. ¹H NMR (500 MHz, DMSO) δ 12.65 (s, 1H), 11.58 (d, J=58.3 Hz, 1H), 10.79 (s, 1H), 9.48 (s, 1H), 8.23 (s, 1H), 7.38 (dd, J=28.9, 12.0 Hz, 3H), 7.24-7.15 (m, 1H), 6.63 (s, 1H), 3.89 (dd, J=8.8, 5.3 Hz, 1H), 3.72 (d, J=45.0 Hz, 2H), 3.52 (s, OH), 3.22-2.95 (m, 1H), 2.81-2.63 (m, 4H), 2.43 (s, 2H), 2.29-2.05 (m, 2H), 1.82 (d, J=12.4 Hz, 2H), 1.59 (t, J=6.4 Hz, 2H), 1.43-1.10 (m, 3H), 1.02 (s, 6H).

### Example 344. (3R)-3-{4-[4-({4-[2-(6,6-dimethyl-1,4,5,7-tetrahydroindazol-3-yl)-1H-indole-6-carbonyl]piperazin-1-yl}methyl)piperidin-1-yl]phenyl}piperidine-2,6-dione (217) and (R)-3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)phenyl)piperidine-2,6-dione (218)

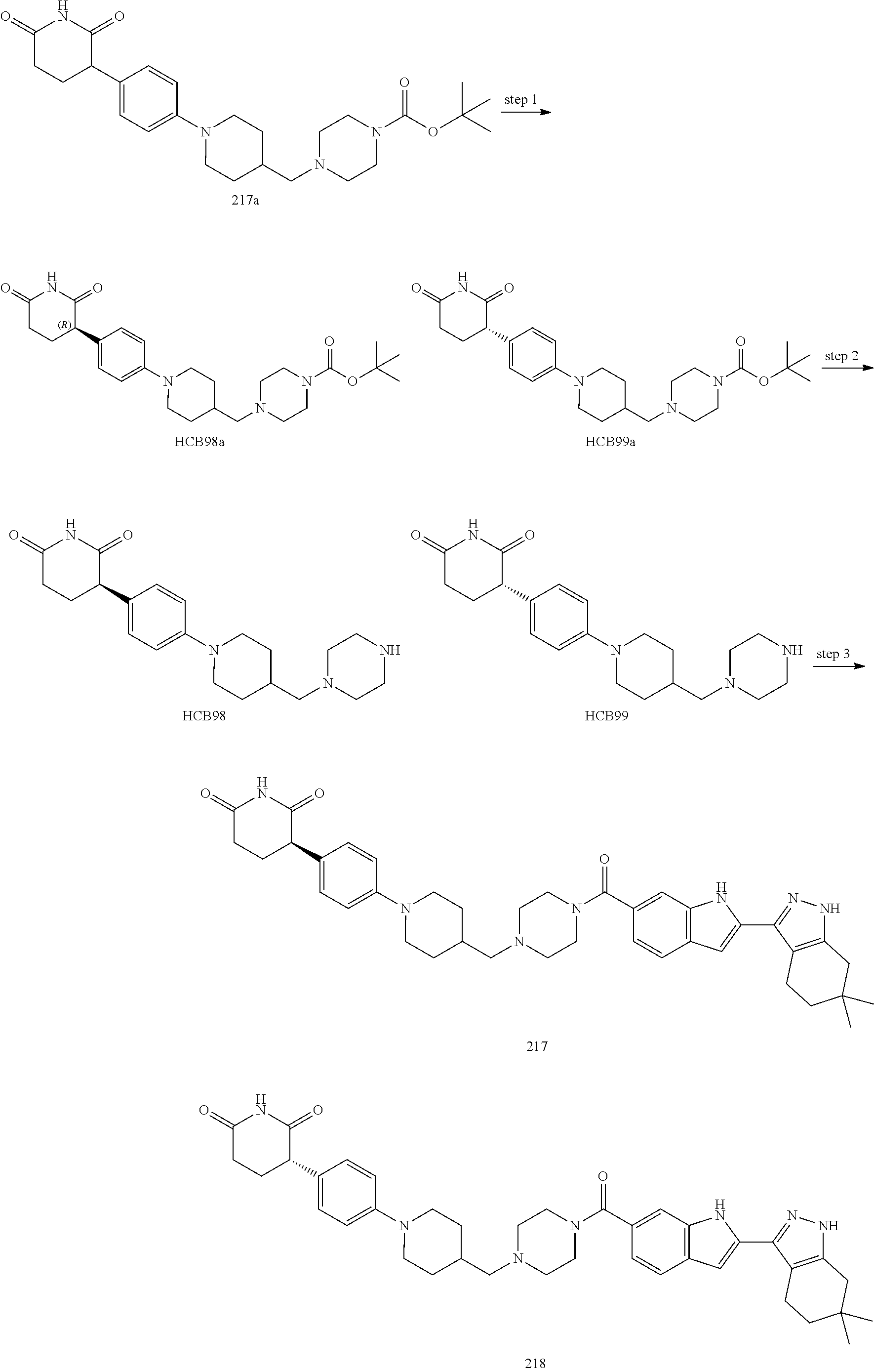

Step 1: Sample preparation—Sample was dissolved in a 2:1 mixture of Acetonitrile and THF into 210 ml at 40° C.˜ 51 mg/ml.
Achiral column—Princeton SFC Diol 15×2 cm, 5 micron
Chiral column—Chiral Technologies UH 15×2 cm, 5 micron
Columns were used in tandem to remove achiral impurity and resolve chiral enantiomers in 1 step.
First Diol column->UH column Last
SFC conditions: i-PrOH: MeCN (1:1)+0.1% NH₄OH, 70 ml/min, 100 bars, 40° C.
   Step 2: tert-butyl 4-[(1-{4-[(3R)-2,6-dioxopiperidin-3-yl]phenyl}piperidin-4-yl)methyl]piperazine-1-carboxylate (HCB98a) (12.54 g, 26.6397 mmol) was dissolved in HFIP (66.00 mL), the mixture was cooled in an ice bath. Once the temperature got down to 10° C., hydrogen chloride 4 M solution in dioxane (66.60 mL, 9.71 g, 266.3967 mmol) was added slowly over about ten minutes. The temperature got up to 30° C. during the addition. A solid gooey mass formed. After an additional 20 minutes, the mixture was concentrated under reduced pressure then pumped to dryness. The resulting solid was dissolved in water (20 ml) and DCM (200 ml) and transferred to a separation funnel. About 20 ml Sat. aq. NaHCO₃ was added. The aqueous layer was extracted 5 times with DCM. The combined organic layers were dried over Na₂SO₄, and concentrated in vacou, (rotovap bath temp below 30° C.) to provide (3R)-3-{4-[4-(piperazin-1-ylmethyl)piperidin-1-yl]phenyl}piperidine-2,6-dione (HCB98) (10 g, 98% chiral purity, 100% yield). ¹H NMR (500 MHz, DMSO-d6) δ 8.05 (d, J=6.5 Hz, 1H), 7.04 (d, J=8.4 Hz, 2H), 6.88 (d, J=8.3 Hz, 2H), 3.72 (dd, J=10.9, 4.9 Hz, 1H), 3.66 (ddt, J=12.7, 7.4, 4.6 Hz, 2H), 2.71 (t, J=4.8 Hz, 4H), 2.63 (tdd, J=12.1, 10.2, 9.4, 4.2 Hz, 4H), 2.48-2.42 (m, 1H), 2.28 (s, 4H), 2.19-2.07 (m, 3H), 2.02 (dt, J=13.2, 5.0 Hz, 1H), 1.84-1.73 (m, 2H), 1.65 (ddd, J=11.6, 7.5, 3.9 Hz, 1H), 1.28-1.11 (m, 2H).

Step 3: To a mixture of (3R)-3-{4-[4-(piperazin-1-ylmethyl)piperidin-1-yl]phenyl}piperidine-2,6-dione (HCB98) (9.50 g, 25.6412 mmol) and 2-(6,6-dimethyl-1,4,5,7-tetrahydroindazol-3-yl)-1H-indole-6-carboxylic acid (BBX₁₉) (7.93 g, 25.6412 mmol) in DMF (60.00 mL) in a room temperature water bath was added ({[3-(dimethylamino)propyl]imino}methylidene)(ethyl)amine hydrochloride (4.92 g, 25.6412 mmol) in 2 portions. After stirring for 3 hours, reaction mixture still not fully dissolved, another 20 ml of DMF was added. After another 3 hours, N,N-diisopropylethylamine (13.40 mL, 9.94 g, 76.9237 mmol) was added. After stirring for 10 minutes, the mixture was poured into 1.2 L ice water. The resulting precipitate was filtered and washed with water. The resulting solid was dissolved in 800 ml of 10% MeOH/DCM. The mixture was transferred to a separation funnel, the aqueous layer was separated. The aqueous layer was washed with 300 ml of 10% MeOH/DCM. The combined organic layers were concentrated to dryness. The crude material was dissolved in 10% MeOH/DCM and concentrated onto silica gel then purified by flash chromatography on 330 g column eluted with 0-10% MeOH/DCM to provide (3R)-3-{4-[4-({4-[2-(6,6-dimethyl-1,4,5,7-tetrahydroindazol-3-yl)-1H-indole-6-carbonyl]piperazin-1-yl}methyl)piperidin-1-yl]phenyl}piperidine-2,6-dione (217) (9.15 g, 53.92%).

### (R)-3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)phenyl)piperidine-2,6-dione (218) (*Arbitrarily Assigned)

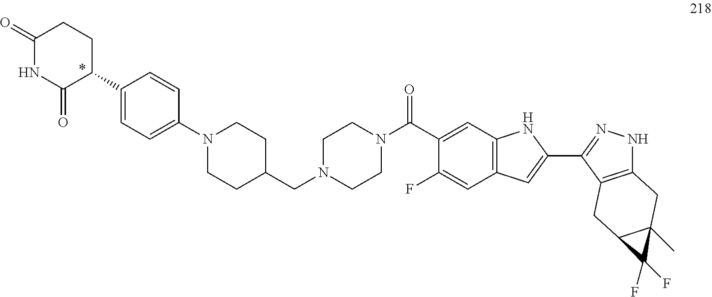 [SMILES: C[C@@]12Cc3[nH]nc(-c4cc5cc(F)c(C(=O)N6CCN(CC7CCN(c8ccc([C@H]9CCC(=O)NC9=O)cc8)CC7)CC6)cc5[nH]4)c3C[C@@H]1C2(F)F]

Followed the procedure described for Compound 217 above starting from (S)-3-(4-(4-(piperazin-1-ylmethyl)piperidin-1-yl)phenyl)piperidine-2,6-dione (HCB99). LCMS: C₃₉H₄₇N₇O₃, required: 661.4, found m/z=662.4 [M+H]⁺. ¹H NMR (500 MHz, DMSO-d6) δ 12.54 (s, 1H), 11.44-11.40 (m, 1H), 10.74 (s, 1H), 7.51 (d, J=8.1 Hz, 1H), 7.43 (s, 1H), 7.04-6.95 (m, 3H), 6.89-6.83 (m, 2H), 6.61-6.57 (m, 1H), 3.69 (dd, J=10.9, 5.0 Hz, 1H), 3.63 (d, J=12.2 Hz, 2H), 3.52 (s, 2H), 2.69-2.56 (m, 5H), 2.47-2.42 (m, 1H), 2.40 (s, 2H), 2.37 (s, 3H), 2.19 (d, J=7.1 Hz, 2H), 2.15-2.04 (m, 1H), 2.03-1.94 (m, 1H), 1.78 (d, J=11.5 Hz, 2H), 1.66 (t, J=3.5, 3.5 Hz, 1H), 1.56 (t, J=6.4, 6.4 Hz, 2H), 1.24-1.13 (m, 2H), 0.99 (s, 6H).

### (S)-3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)phenyl)piperidine-2,6-dione (219) (*Arbitrarily Assigned)

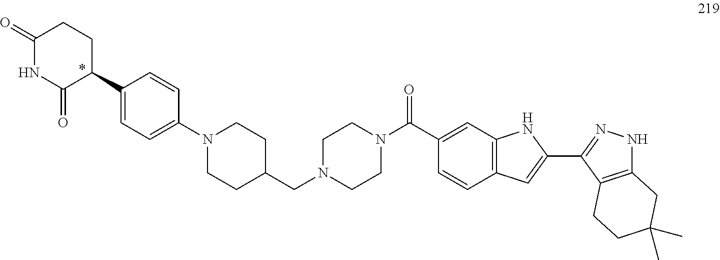 [SMILES: CC1(C)CCc2c(-c3cc4ccc(C(=O)N5CCN(CC6CCN(c7ccc([C@@H]8CCC(=O)NC8=O)cc7)CC6)CC5)cc4[nH]3)n[nH]c2C1]

Followed the procedure described for Compound 217 above starting from tert-butyl (S)-4-((1-(4-(2,6-dioxopiperidin-3-yl)phenyl)piperidin-4-yl)methyl)piperazine-1-carboxylate. LCMS: C₃₉H₄₇N₇O₃, required: 661.4, found m/z=662.6 [M+H]⁺. ¹H NMR (500 MHz, DMSO-d6) δ 12.54 (s, 1H), 11.42 (s, 1H), 10.74 (s, 1H), 7.51 (d, J=8.2 Hz, 1H), 7.43 (s, 1H), 7.04-6.95 (m, 3H), 6.89-6.83 (m, 2H), 6.61-6.57 (m, 1H), 5.74 (s, 1H), 3.69 (dd, J=10.9, 4.9 Hz, 1H), 3.63 (d, J=12.2 Hz, 2H), 3.52 (s, 2H), 2.68-2.56 (m, 5H), 2.47-2.41 (m, 2H), 2.39 (d, J=14.0 Hz, 6H), 2.19 (d, J=7.3 Hz, 2H), 2.14-2.05 (m, 1H), 2.02-1.95 (m, 1H), 1.78 (d, J=12.0 Hz, 2H), 1.68-1.61 (m, 1H), 1.57 (t, J=6.4, 6.4 Hz, 2H), 1.25-1.14 (m, 3H), 1.00 (s, 6H).

### Example 345. (R)-3-(4-(4-((4-(2-((4aS,5aR)-5,5-difluoro-5a-methyl-1,4,4a,5,5a,6-hexahydrocyclopropa[f]indazol-3-yl)-5-fluoro-1H-indole-6-carbonyl)piperazin-1-yl)methyl)piperidin-1-yl)phenyl)piperidine-2,6-dione (220) (*Arbitrarily Assigned)

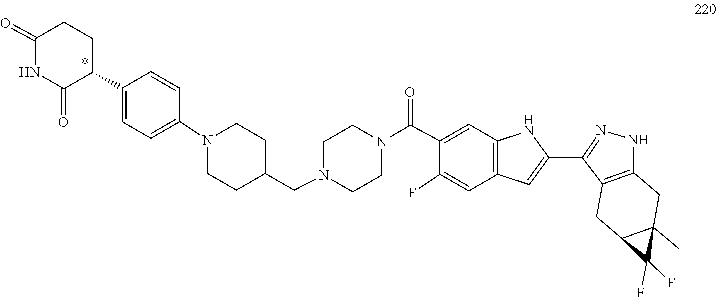 [SMILES: C[C@@]12Cc3[nH]nc(-c4cc5cc(F)c(C(=O)N6CCN(CC7CCN(c8ccc([C@H]9CCC(=O)NC9=O)cc8)CC7)CC6)cc5[nH]4)c3C[C@@H]1C2(F)F]

Followed the procedure described for Compound 217 starting from 2-((4aS,5aR)-5,5-difluoro-5a-methyl-1,4,4a,5,5a,6-hexahydrocyclopropa[f]indazol-3-yl)-5-fluoro-1H-indole-6-carboxylic acid. LCMS: C₃₉H₄₂F₃N₇O₃ requires: 713, found: m/z=714 [M+H]⁺. ¹H NMR (400 MHz, DMSO-d₆) δ 12.75 (s, 1H), 11.55 (s, 1H), 10.77 (s, 1H), 7.38-7.27 (m, 2H), 7.06-6.99 (m, 2H), 6.91-6.84 (m, 2H), 6.62 (s, 1H), 3.75-3.61 (m, 5H), 3.31-3.23 (m, 2H), 3.17-3.03 (m, 3H), 2.89-2.80 (m, 1H), 2.65-2.56 (m, 3H), 2.50-2.40 (m, 3H), 2.32 (s, 2H), 2.23-2.17 (m, 2H), 2.16-2.09 (m, 1H), 2.06-1.96 (m, 1H), 1.93-1.84 (m, 1H) 1.83-1.76 (m, 2H), 1.66 (s, 1H), 1.39 (s, 3H), 1.26-1.13 (m, 2H).

### Example 346. 3-(4-(4-((4-(2-(6,6-dimethyl-4,5,6,7-tetrahydro-1H-indazol-3-yl)-1H-indole-6-carbonyl)piperazin-1-yl)methyl)piperidin-1-yl)-2-fluorophenyl)piperidine-2,6-dione (221)

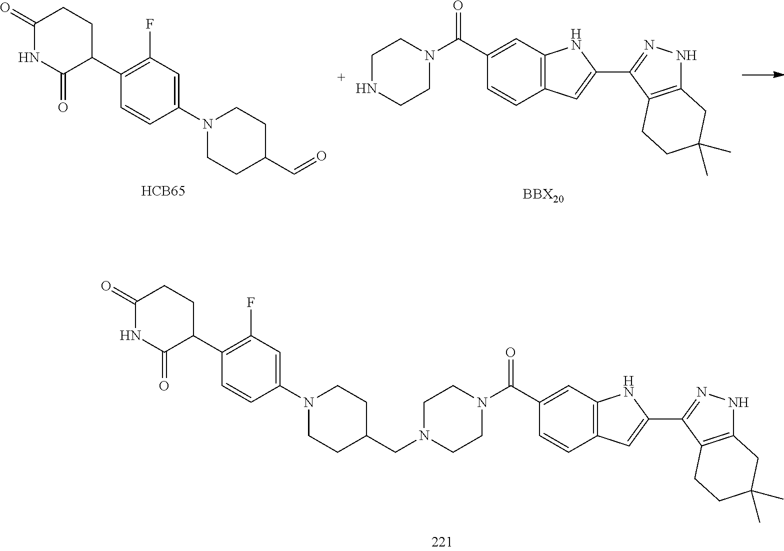

To a solution of 3-{4-[4-({4-[2-(6,6-dimethyl-1,4,5,7-tetrahydroindazol-3-yl)-1H-indole-6-carbonyl]piperazin-1-yl}methyl)piperidin-1-yl]-2-fluorophenyl}piperidine-2,6-dione (0.0068 g, 21.5%) and 6,6-dimethyl-3-[6-(piperazine-1-carbonyl)-1H-indol-2-yl]-1,4,5,7-tetrahydroindazole (BBX₂₀) (17.55 mg, 0.05 mmol) in DCM, N,N-diisopropylethylamine (24.3 uL, 0.14 mmol) was added, then sodium triacetoxyborohydride (29.6 mg, 0.14 mmol) was added, the reaction mixture was stirred for 1 hour. The reaction mixture was poured into water, extracted with DCM twice. The organic solution was dried over Na₂SO₄ and concentrated. ISCO silica gel column purification eluted with MeOH in DCM (0-12%) to afford 3-{4-[4-({4-[2-(6,6-dimethyl-1,4,5,7-tetrahydroindazol-3-yl)-1H-indole-6-carbonyl]piperazin-1-yl}methyl)piperidin-1-yl]-2-fluorophenyl}piperidine-2,6-dione (6.8 mg, 21.5%). LCMS C₃₉H₄₆FN₇O₃ required: 679.4, found: m/z=680.6 [M+H]⁺. ¹H NMR (500 MHz, CD₃CN) δ 9.93 (s, 1H), 8.73 (s, 1H), 7.65 (d, J=8.1 Hz, 1H), 7.58 (s, 1H), 7.16 (d, J=8.1 Hz, 1H), 7.09 (t, J=8.8 Hz, 1H), 0.78-6.61 (m, 3H), 3.85 (dd, J=12.4, 5.0 Hz, 1H), 3.78-3.68 (m, 2H), 3.01 (d, J=6.8 Hz, 2H), 2.84-2.73 (m, 4H), 2.71-2.58 (m, 2H), 2.48 (s, 2H), 1.92 (d, J=13.9 Hz, 4H), 1.67 (t, J=6.4 Hz, 2H), 1.39 (qd, J=12.4, 3.9 Hz, 2H), 1.07 (s, 6H).

### Example 347. 3-{4-[4-({4-[2-(6,6-dimethyl-1,4,5,7-tetrahydroindazol-3-yl)-1H-indole-6-carbonyl]piperazin-1-yl}methyl)piperidin-1-yl]-3-fluorophenyl}piperidine-2,6-dione (222)

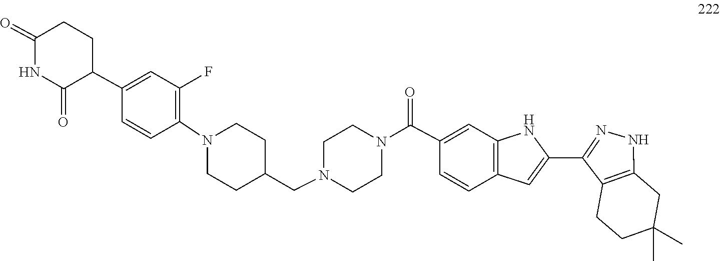 [SMILES: CC1(C)CCc2c(-c3cc4ccc(C(=O)N5CCN(CC6CCN(c7ccc(C8CCC(=O)NC8=O)cc7F)CC6)CC5)cc4[nH]3)n[nH]c2C1]

This compound was synthesized following the same procedure as Compound 221.

LCMS: C₃₉H₄₆FN₇O₃ required: 679.4, found: m/z=680. ¹H NMR (500 MHz, DMSO) δ 11.54 (s, 1H), 10.83 (s, 1H), 9.47 (s, 1H), 7.58 (d, J=8.1 Hz, 1H), 7.55 (s, 1H), 7.10 (d, J=8.1 Hz, 1H), 7.07-6.94 (m, 3H), 6.66 (d, J=2.0 Hz, 1H), 3.81 (dd, J=11.8, 4.9 Hz, 1H), 3.54 (d, J=23.6 Hz, 3H), 3.14 (s, 4H), 2.75-2.59 (m, 5H), 2.43 (s, 2H), 2.20 (qd, J=12.6, 4.6 Hz, 1H), 2.05-1.93 (m, 2H), 1.86 (d, J=12.3 Hz, 2H), 1.60 (t, J=6.4 Hz, 2H), 1.42 (d, J=12.1 Hz, 2H), 1.02 (s, 6H).

### Example 348. 1-{[1-(2-{4-[2-(6,6-dimethyl-1,4,5,7-tetrahydroindazol-3-yl)-1H-indole-6-carbonyl]piperazin-1-yl}-2-oxoethyl)pyrazol-4-yl]methyl}-1,3-diazinane-2,4-dione (223)

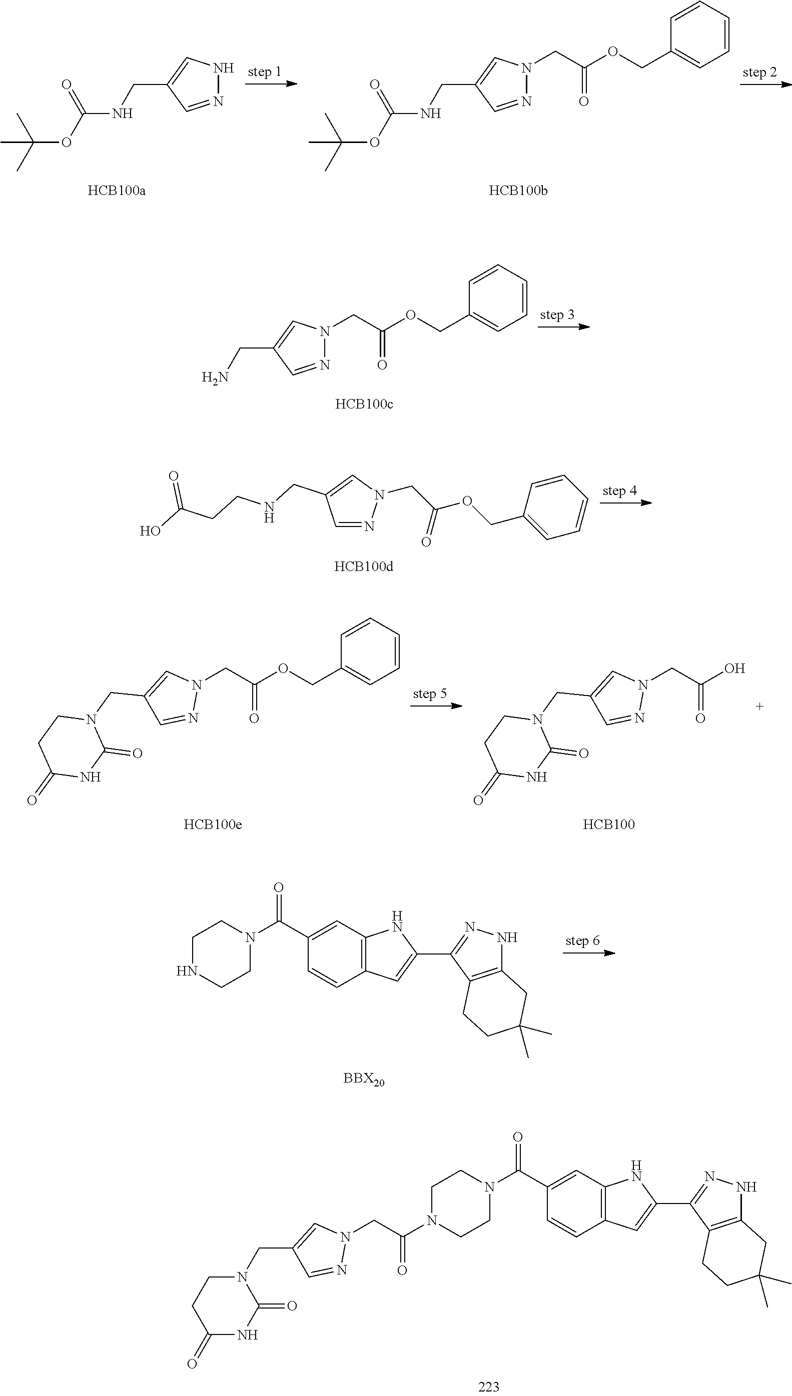

Step 1: to a solution of tert-butyl N-(1H-pyrazol-4-ylmethyl)carbamate (1.00 g, mmol) and caesium carbonate (1.98 g, 6.08 mmol) in 10 ml acetonitrile, benzyl 2-bromoacetate (1.28 g, 5.59 mmol) in 5 ml acetonitrile was added, the solution was stirred for 30 minutes, then heated at 60° C. overnight. The solution was cooled down, solvent was evaporated under reduced pressure. Re-dissolved the crude product by EtOAc, washed the organic solution by water and brine, dried over Na₂SO₄ and concentrated. The crude product was purified by ISCO silica gel chromatography eluted with EtOAc/hexane (5-100%) to afford benzyl 2-(4-{[(tert-butoxycarbonyl)amino]methyl}pyrazol-1-yl)acetate (1.71 g, 100% yield). LCMS: C₁₈H₂₃N₃O₄ requires: 345.2, found: m/z=346.2. ¹H NMR (500 MHz, CDCl3) δ 7.52 (s, 1H), 7.45 (s, 1H), 7.43-7.33 (m, 5H), 5.23 (s, 2H), 4.94 (s, 2H), 4.22 (d, J=5.7 Hz, 2H), 1.48 (s, 9H).

Step 2: Benzyl 2-(4-{[(tert-butoxycarbonyl)amino]methyl}pyrazol-1-yl)acetate (1.7 g, 5.18 mmol) was dissolved in 20 ml 5% TFA in HFIP, the reaction mixture was stirred for 1 hour. The solution was evaporated under reduced pressure. Re-dissolved the product in EtOAc, washed organic solution with 0.1 N NH₄OH solution (2 ml) and brine. The organic solution was dried over Na₂SO₄ and concentrated. Moved to the next step without purification. LCMS: C₁₃H₁₅N₃O₂ requires: 245.1, found: m/z=246.2.

Step 3 and step 4: To a solution of benzyl 2-[4-(aminomethyl)pyrazol-1-yl]acetate (530.00 mg, 2.16 mmol) in Dioxane (4.00 mL) and DMF (2 ml), triethylamine (1.21 mL, 0.87 g, 8.64 mmol) was added, then acrylic acid (155.71 mg, 2.16 mmol) was added, the reaction was heated at 70° C. for 16 hours, evaporated all the solvent. Re-dissolved the crude product in 10 ml acetic acid, urea (374.30 mg, 6.23 mmol) was added, heated the solution at 120° C. for 16 hours. The solvent was evaporated under reduced pressure. The crude product was purified by ISCO silica gel chromatography eluted with EtOAc in hexane (10-100%) to afford benzyl 2-{4-[(2,4-dioxo-1,3-diazinan-1-yl)methyl]pyrazol-1-yl}acetate (0.172 g, 23.3%). LCMS C₁₇H₁₈N₄O₄ requires: 342.1 found: m/z=343.2 [M+H]⁺.

Step 5: To a solution of benzyl 2-{4-[(2,4-dioxo-1,3-diazinan-1-yl)methyl]pyrazol-1-yl}acetate (62.00 mg, 0.1811 mmol) in 1 ml MeOH and 1 ml THF, lithium (1+) hydrate hydroxide (15.11 mg, 0.3600 mmol) in 1 ml water was added, the solution was stirred for 1 hour. Evaporated all the solvent, the product was lyophilized to afford {4-[(2,4-dioxo-1,3-diazinan-1-yl)methyl]pyrazol-1-yl}acetic acid lithium salt, quantitative yield.

Step 6: {4-[(2,4-dioxo-1,3-diazinan-1-yl)methyl]pyrazol-1-yl}acetic acid (HCB100) (7.00 mg, 0.0278 mmol) and 6,6-dimethyl-3-[6-(piperazine-1-carbonyl)-1H-indol-2-yl]-1,4,5,7-tetrahydroindazole (BBX₂₀) (10.48 mg, 0.0278 mmol) in 0.2 ml DMF, N,N-diisopropylethylamine (14.95 uL, 10.76 mg, 0.0833 mmol) was added, followed by addition of [(dimethylamino)({[1,2,3]triazolo[4,5-b]pyridin-3-yloxy})methylidene]dimethylazanium; hexafluoro-lambda5-phosphanuide (10.55 mg, 0.0278 mmol) in 0.1 ml DMF, the reaction was stirred for 1 hour. The reaction mixture was quenched by 1 ml water, extracted by DCM for three times. The combined organic solution was dried over Na₂SO₄ and concentrated. The crude product was purified by ISCO silica gel chromatography eluted with MEOH in DCM (0-20%) to afford 1-{[1-(2-{4-[2-(6,6-dimethyl-1,4,5,7-tetrahydroindazol-3-yl)-1H-indole-6-carbonyl]piperazin-1-yl}-2-oxoethyl)pyrazol-4-yl]methyl}-1,3-diazinane-2,4-dione (1.6 mg, 9.4%). LCMS: C₃₂H₃₇N₉O₄ requires: 611.3, found: m/z=612.5. ¹H NMR (500 MHz, CD₃CN) δ 7.63 (d, J=8.1 Hz, 1H), 7.53 (d, J=2.5 Hz, 2H), 7.42 (s, 1H), 7.13 (d, J=8.2 Hz, 1H), 6.74 (d, J=2.0 Hz, 1H), 5.02 (s, 2H), 4.45 (s, 2H), 3.68 (pd, J=6.7, 3.9 Hz, 2H), 3.57 (d, J=34.4 Hz, 4H), 3.39 (t, J=6.8 Hz, 2H), 3.15 (qd, J=7.4, 4.3 Hz, 2H), 2.78 (t, J=6.4 Hz, 2H), 2.58 (t, J=6.8 Hz, 2H), 2.49 (s, 2H), 1.67 (t, J=6.4 Hz, 2H), 1.07 (s, 6H).

### Example 349. (3RS)-3-(4-{4-[(4-{2-[(6S)-6-(methoxymethyl)-6-methyl-1,4,5,7-tetrahydroindazol-3-yl]-1H-indole-6-carbonyl}piperazin-1-yl)methyl]piperidin-1-yl}phenyl)piperidine-2,6-dione (224) (*Arbitrarily Assigned)

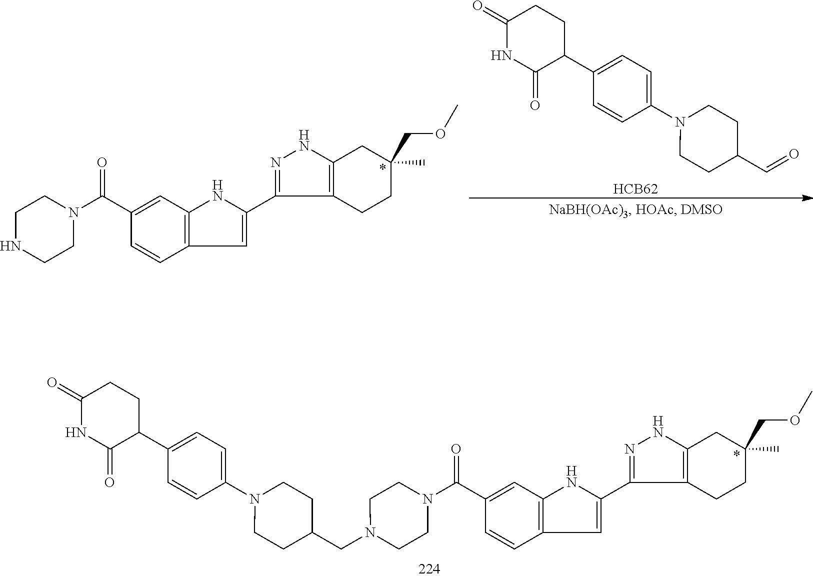 [SMILES: COC[C@@]1(C)CCc2c(-c3cc4ccc(C(=O)N5CCNCC5)cc4[nH]3)n[nH]c2C1 ; "HCB62" ; O=CC1CCN(c2ccc(C3CCC(=O)NC3=O)cc2)CC1 ; COC[C@@]1(C)CCc2c(-c3cc4ccc(C(=O)N5CCN(CC6CCN(c7ccc(C8CCC(=O)NC8=O)cc7)CC6)CC5)cc4[nH]3)n[nH]c2C1]

To a stirred mixture of (6S)-6-(methoxymethyl)-6-methyl-3-[6-(piperazine-1-carbonyl)-1H-indol-2-yl]-1,4,5,7-tetrahydroindazole (100 mg, 0.245 mmol, 1 equiv) in DMSO (1 mL) was added DIEA (0.5 mL) at room temperature. The resulting mixture was stirred for 15 min at room temperature. To the above mixture was added 1-[4-(2,6-dioxopiperidin-3-yl)phenyl]piperidine-4-carbaldehyde (HCB62) (73.7 mg, 0.245 mmol, 1 equiv) and HOAc (0.5 mL) at room temperature. The resulting mixture was stirred for 30 min at room temperature. To the above mixture was added NaBH(OAc)₃ (104.02 mg, 0.490 mmol, 2 equiv) at 0° C. The resulting mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The residue was purified by silica gel column chromatography, eluted with CH₃CN/H₂O (1/1) to afford (3RS)-3-(4-{4-[(4-{2-[(6S)-6-(methoxymethyl)-6-methyl-1,4,5,7-tetrahydroindazol-3-yl]-1H-indole-6-carbonyl}piperazin-1-yl)methyl]piperidin-1-yl}phenyl)piperidine-2,6-dione (36.8 mg, 20.72%) as an off-white solid. MS (ESI) calc'd for (C₄₀H₄₉N₇O₄)[M+1]⁺, 692.4; found, 692.3. ¹H NMR (400 MHz, DMSO-d₆) δ 12.56 (s, 1H), 11.43 (s, 1H), 10.76 (s, 1H), 7.54 (d, J=8.0 Hz, 1H), 7.45 (s, 1H), 7.04-7.02 (m, 3H), 6.99-6.89 (m, 2H), 6.62 (s, 1H), 3.73-3.67 (m, 3H), 3.54 (s, 3H), 3.29 (s, 3H), 3.19 (s, 2H), 2.68-2.58 (m, 7H), 2.46-2.31 (m, 5H), 2.31-2.22 (m, 2H), 2.22-2.19 (m, 1H), 2.10-2.01 (m, 1H), 1.82-1.71 (m, 2H), 1.79-1.70 (m, 2H), 1.69-1.60 (m, 1H), 1.24-1.20 (m, 3H), 0.98 (s, 3H).

### Example 350. (3RS)-3-(4-{4-[(4-{2-[(4aS,5aR)-5a-Methyl-1H,4H,4aH,5H,6H-cyclopropa[f]indazol-3-yl]-1H-indole-6-carbonyl}piperazin-1-yl)methyl]piperidin-1-yl}phenyl)piperidine-2,6-dione (225)

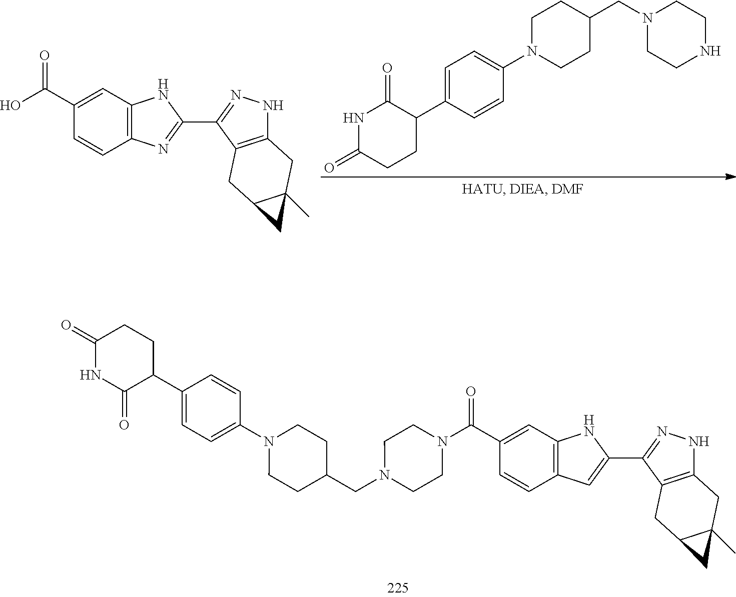

To a mixture of 2-[(4aS,5aR)-5a-methyl-1H,4H,4aH,5H,6H-cyclopropa[f]indazol-3-yl]-1H-indole-6-carboxylic acid (40 mg, 0.130 mmol, 1 equiv) and 3-{4-[4-(piperazin-1-ylmethyl)piperidin-1-yl]phenyl}piperidine-2,6-dione (72.3 mg, 0.195 mmol, 1.5 equiv) in DMF (1 mL) were added HATU (74.2 mg, 0.195 mmol, 1.5 equiv) and DIEA (50.5 mg, 0.390 mmol, 3 equiv). The resulting mixture was stirred for 1 h at room temperature. The resulting mixture was extracted with EtOAc. The combined organic layers were washed with water, dried over anhydrous Na₂SO₄. After filtration, the filtrate was concentrated under reduced pressure. The residue was purified by silica gel column chromatography, eluted with CH₃CN/H₂O (50/50) to afford (3RS)-3-(4-{4-[(4-{2-[(4aS,5aR)-5a-methyl-1H,4H,4aH,5H,6H-cyclopropa[f]indazol-3-yl]-1H-indole-6-carbonyl}piperazin-1-yl)methyl]piperidin-1-yl}phenyl)piperidine-2,6-dione (18.3 mg, 20.78%) as an off-white solid. MS (ESI) calc'd for (C₃₉H₄₅N₇O₃)[M+1]⁺, 660.3; found, 660.4. ¹H NMR (400 MHz, Methanol-d₄) δ 7.63 (d, J=8.2 Hz, 1H), 7.53 (s, 1H), 7.13-7.09 (m, 3H), 7.00-6.97 (m, 2H), 6.70 (s, 1H), 3.86-3.76 (m, 7H), 3.22-3.03 (m, 3H), 2.79 (s, 1H), 2.78-2.61 (m, 3H), 2.58-2.51 (m, 4H), 2.32-2.30 (m, 2H), 2.22-2.20 (m, 2H), 1.93-1.89 (m, 2H), 1.89-1.74 (m, 1H), 1.45-1.34 (m, 2H), 1.20-1.18 (m, 3H), 1.34-1.30 (m, 1H), 1.01-0.91 (m, 1H), 0.49-0.40 (m, 1H), 0.31-0.24 (m, 1H).

### Example 351. (3RS)-3-(4-{4-[(4-{2-[(4aS,5aR)-5a-methyl-1H,4H,4aH,5H,6H-cyclopropa[f]indazol-3-yl]-3H-1,3-benzodiazole-5-carbonyl}piperazin-1-yl)methyl]piperidin-1-yl}phenyl)piperidine-2,6-dione (226)

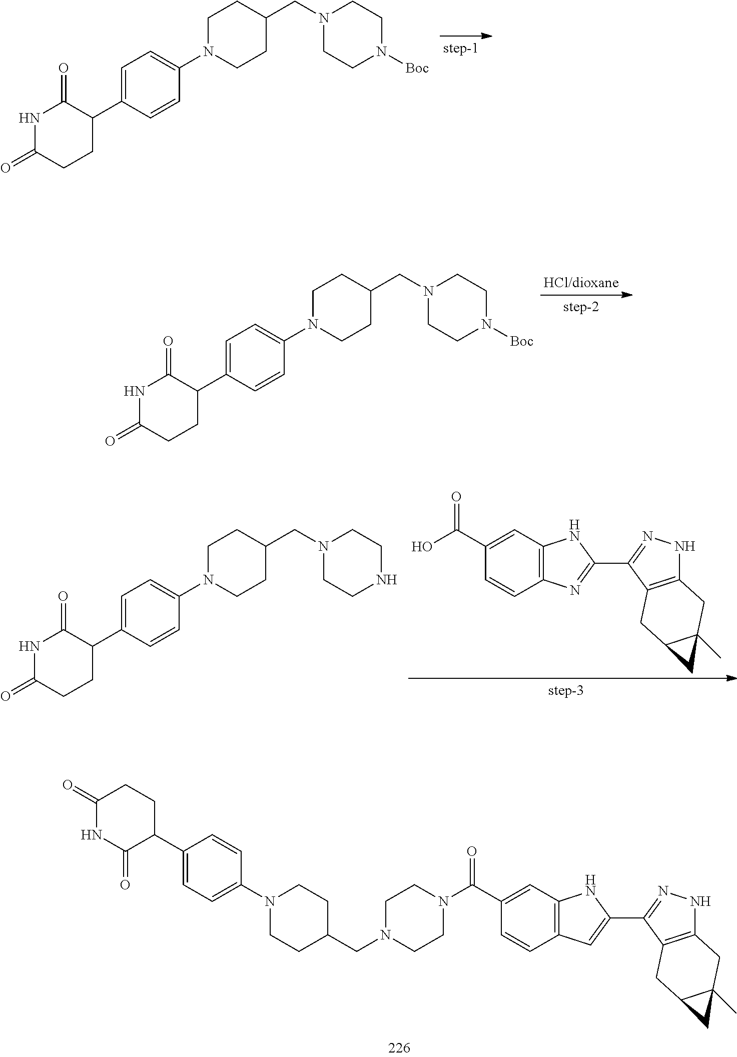

### Step 1: Synthesis of tert-butyl 4-({1-[4-(2,6-dioxopiperidin-3-yl)phenyl]piperidin-4-yl}methyl)piperazine-1-carboxylate

To a mixture of tert-butyl 4-[(1-{4-[(3R)-2,6-dioxopiperidin-3-yl]phenyl}piperidin-4-yl)methyl]piperazine-1-carboxylate (1 g, 2.125 mmol, 1 equiv) in dioxane (100 mL) was added TEA (100 mL) at room temperature under nitrogen atmosphere. The resulting mixture was stirred at 80° C. under nitrogen atmosphere overnight. The resulting mixture was concentrated under vacuum. This resulted in tert-butyl 4-({1-[4-(2,6-dioxopiperidin-3-yl)phenyl]piperidin-4-yl}methyl)piperazine-1-carboxylate (1 g, crude) as a white solid.. MS (ESI) calc'd for (C₂₆H₃₈N₄O₄) [M+1]⁺, 471.3; found, 471.2

### Step 2: Synthesis of 3-(4-(4-(piperazin-1-ylmethyl)piperidin-1-yl)phenyl)piperidine-2,6-dione

To a mixture of tert-butyl 4-((1-(4-(2,6-dioxopiperidin-3-yl)phenyl)piperidin-4-yl)methyl)piperazine-1-carboxylate (100 mg, 0.212 mmol, 1 equiv) in HFIP (0.25 mL) was added HCl (gas) in 1,4-dioxane (5 mL) dropwise at −10° C. The resulting mixture was stirred for 30 min at −10° C. The resulting mixture was concentrated under vacuum at 0° C. The resulting mixture was extracted with CH₂Cl₂. The combined organic layers were washed with NaHCO₃ (3N) aqueous, dried over anhydrous Na₂SO₄. After filtration, the filtrate was concentrated under reduced pressure. This resulted in 3-(4-(4-(piperazin-1-ylmethyl)piperidin-1-yl)phenyl)piperidine-2,6-dione (74 mg, crude) as an off-white solid. MS (ESI) calc'd for (C₂₁H₃₀N₄O₂) [M+1]⁺, 371.2; found, 371.1 [M+H]⁺.

### Step 3: Synthesis of (3RS)-3-(4-{4-[(4-{2-[(4aS,5aR)-5a-methyl-1H,4H,4aH,5H,6H-cyclopropa[f]indazol-3-yl]-3H-1,3-benzodiazole-5-carbonyl}piperazin-1-yl)methyl]piperidin-1-yl}phenyl)piperidine-2,6-dione

To a mixture of 2-[(4aS,5aR)-5a-methyl-1H,4H,4aH,5H,6H-cyclopropa[f]indazol-3-yl]-3H-1,3-benzodiazole-5-carboxylic acid (50 mg, 0.162 mmol, 1 equiv), 3-{4-[4-(piperazin-1-ylmethyl)piperidin-1-yl]phenyl}piperidine-2,6-dione (72.1 mg, 0.194 mmol, 1.2 equiv) and HATU (92.5 mg, 0.243 mmol, 1.5 equiv) in DMF (5 mL) was added DIEA (62.9 mg, 0.4860 mmol, 3 equiv) at room temperature. The resulting mixture was stirred for 1 h at room temperature. The resulting mixture was extracted with EtOAc. The combined organic layers were washed with water, dried over anhydrous Na₂SO₄. After filtration, the filtrate was concentrated under reduced pressure. The residue was purified by reverse phase flash with the following conditions (MeCN/H₂O=40/50) to afford (3RS)-3-(4-{4-[(4-{2-[(4aS,5aR)-5a-methyl-1H,4H,4aH,5H,6H-cyclopropa[f]indazol-3-yl]-3H-1,3-benzodiazole-5-carbonyl}piperazin-1-yl)methyl]piperidin-1-yl}phenyl)piperidine-2,6-dione (28.8 mg, 25.69%) as a white solid. MS (ESI) calc'd for (C₃₈H₄₄N₈O₃)[M+1]⁺, 661.4; found, 661.4. ¹H NMR (400 MHz, DMSO-d₆) δ 12.83 (s, 2H), 10.76 (s, 1H), 7.54 (s, 2H), 7.19 (d, J=8.4 Hz, 1H), 7.03 (d, J=8.2 Hz, 2H), 6.88 (d, J=8.4 Hz, 2H), 3.73-3.64 (m, 3H), 3.53 (s, 3H), 3.46-3.41 (m, 1H), 3.03-2.99 (m, 2H), 2.76-2.72 (m, 1H), 2.70-2.57 (m, 3H), 2.53-2.50 (m, 2H), 2.44-2.39 (m, 4H), 2.22-2.20 (m, 2H), 2.13-2.10 (m, 1H), 2.03-1.99 (m, 1H), 1.81-1.78 (m, 2H), 1.68 (s, 1H), 1.25-1.20 (m, 5H), 1.13-1.10 (m, 1H), 0.39-0.36 (m, 1H), 0.18-0.16 (m, 1H).

### Example 352. (3RS)-3-(4-{4-[(4-{2-[(4aS,5aR)-5,5-difluoro-5a-methyl-1H,4H,4aH,6H-cyclopropa [f]indazol-3-yl]-3H-1,3-benzodiazole-5-carbonyl}piperazin-1-yl)methyl]piperidin-1-yl}phenyl)piperidine-2,6-dione (227)

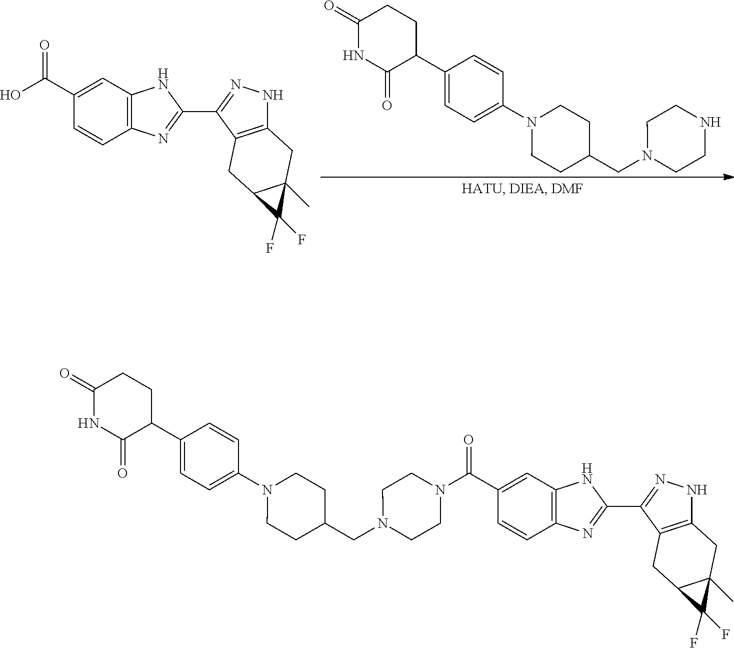 [SMILES: C[C@@]12Cc3[nH]nc(-c4nc5ccc(C(=O)O)cc5[nH]4)c3C[C@@H]1C2(F)F ; O=C1CCC(c2ccc(N3CCC(CN4CCNCC4)CC3)cc2)C(=O)N1 ; C[C@@]12Cc3[nH]nc(-c4nc5ccc(C(=O)N6CCN(CC7CCN(c8ccc(C9CCC(=O)NC9=O)cc8)CC7)CC6)cc5[nH]4)c3C[C@@H]1C2(F)F]

To a mixture of 2-[(4aS,5aR)-5,5-difluoro-5a-methyl-1H,4H,4aH,6H-cyclopropa[f] indazol-3-yl]-3H-1,3-benzodiazole-5-carboxylic acid (50 mg, 0.145 mmol, 1 equiv) and 3-{4-[4-(piperazin-1-ylmethyl)piperidin-1-yl] phenyl}piperidine-2,6-dione (64.56 mg, 0.174 mmol, 1.2 equiv) in DMF (2 mL) were added HATU (82.82 mg, 0.217 mmol, 1.5 equiv) and DIEA (56.30 mg, 0.435 mmol, 3.0 equiv) at room temperature. The resulting mixture was stirred for 2 h at room temperature. The resulting mixture was extracted with EtOAc. The combined organic layers were washed with water, dried over anhydrous Na₂SO₄. After filtration, the filtrate was concentrated under reduced pressure. The residue The residue was purified by silica gel column chromatography, eluted with CH₃CN/H₂O (50/50) to afford (3R,S)-3-(4-{4-[(4-{2-[(4aS,5aR)-5,5-difluoro-5a-methyl-1H,4H,4aH,6H-cyclopropa[f]indazol-3-yl]-3H-1,3-benzodiazole-5-carbonyl}piperazin-1-yl)methyl]piperidin-1-yl}phenyl)piperidine-2,6-dione (29.4 mg, 72.91%) as a white solid. MS (ESI) calc'd for (C₃₇H₄₃FN₈O₃)[M+1]⁺, 697.3; found, 697.3. ¹H NMR (400 MHz, DMSO-d₆) δ 12.98 (d, J=4.0 Hz, 1H), 12.80 (d, J=8.4 Hz, 1H), 10.76 (s, 1H), 7.69-7.60 (m, 1H), 7.48 (d, J=8.8 Hz, 1H), 7.24-7.22 (m, 1H), 7.03 (d, J=8.4 Hz, 2H), 6.88 (d, J=8.4 Hz, 2H), 3.73-3.70 (m, 1H), 3.69-3.64 (m, 2H), 3.54 (s, 4H), 3.27 (s, 2H), 3.10-3.06 (m, 1H), 2.89-2.85 (m, 1H), 2.72-2.57 (m, 3H), 2.48-2.43 (m 1H), 2.42-2.40 (m, 4H), 2.22-2.20 (m, 2H), 2.18-2.06 (m, 1H), 2.01-1.99 (m, 1H), 1.92-1.76 (m, 3H), 1.72-1.60 (m, 1H), 1.39 (s, 3H), 1.30-1.14 (m, 2H).

### Biological Example 1—In Vitro ITK Degradation

### Itk Degradation Hibit Assay

Compounds provided herein were assayed in vitro with ITK HiBit cell lines. Compound dilution series (11-point, 5 fold dilutions in DMSO, columns 2-12 with replicate in rows A/B, C/D, E/F, G,H at 2000× the final required concentrations were prepared in 96 well plates (Falcon, cat. no. 353077). Column 1, rows A-H were control DMSO. The 2000× solutions ranged from 2 mM to 1.024 nM (final assay concentration range 1 μM to 0.512 μM). The 2000× solutions were added to cells in 10 μL volume, for a final DMSO concentration of 0.5% and final assay compound concentration of 1×. For the cells, C-terminal HiBiT-tagged Molt4 cells (ATCC CRL-1552, monoclonal cell line clone 1C₁₀) were plated at 1×10⁶ cells/mL, 100 μL/well (100×10⁴ cells/well) in complete RPMI (10% FBS, 1% L-glutamine). The cells were incubated with compound 10 and compound 17 for 4 hrs at 32° C./6% CO₂.

Following incubation, 100 μL of complete Nano-Glo HiBiT Lytic Detection Reagent (Nano-Glo HiBiT Lytic Buffer with 1:50 Nano-Glo HiBiT Lytic Substrate and 1:100 LgBiT Protein; Promega cat. no. N₃₀₄₀) was added. Cells were further incubated for 10 min at room temperature (RT). Luminescence units (LU) were read on an EnVision plate reader (Perkin Elmer, 0.1 sec per well). Percent ITK remaining per sample was calculated as follows:

$$\% ITK remaining = 100 - [ \frac{Control LU - Sample LU}{Control LU} ] \times 100$$

Using Graphpad Prism, % ITK remaining values were plotted as a function of compound concentration. To determine DC₅₀ and Dₘₐₓ values, resulting curves were fit to the Prism curve-fitting equation “log(inhibitor) vs response—Variable slope (four parameters)” (reported best fit value IC₅₀ used as DC₅₀). ITK was measured with antibody-based MSD (Meso-Scale Discovery) assays shown below.

### ITK Degradation MSD (Meso Scale Discovery) Assay

Compound dilution series (7-point, 5 fold dilutions in DMSO, rows B-H with replicate in Column ½, ¾, ⅚, 7,8, at 2000× the final required concentrations were prepared in 96 well plates (Falcon, cat. no. 353077). Row A, Column 1-8 were control DMSO. The 2000× solutions ranged from 2 mM to 128 nM (final assay concentration range 1 μM to 64 M). The 2000× solutions were added to cells in 10 μL volume, for a final DMSO concentration of 0.5% and final assay compound concentration of 1×. For the cells, either human Jurkat (Clone E6-1 ATCC TIB-152) or Motl4 (ATCC CRL-1552), were plated at 1×10⁶ cells/mL, 100 μL/well (100×10⁴ cells/well) in complete RPMI (10% FBS, 1% L-glutamine). Cells were incubated for 4 or 6 hrs at 32° C./6% CO₂.

Following incubation, plates were centrifuged at 1200 rpm for 5 min. supernatant was removed and 50 μL of cell lysis buffer ((MSD Tris lysis buffer (R60TX), Complete Mini EDTA-free protease inhibitor (Sigma 11836170001), Protease Inhibitor Cocktail (Sigma, P2714), Phosphatase Inhibitor Cocktail 2 and 3 (Sigma, P5726 and P0044), and Benzonase (Sigma, E1014)) was added to each well. Plates were sealed and shook at 4° C. for 30 min. Plates were centrifuged and 45 μL was removed for plate assays.

Assay plate preparation: Meso Scale Discovery (MSD) multi-array sm spot 96 well plates (Goat anti-Rabbit L⁴⁵-RA), were blocked with 3% BSA blocking buffer (3% Bovine Serum Albumin (Sigma A3059)+TBS 0.2% Tween-20) for 1 hr with gentle rocking at room temperature. Plates were then washed with 200 μL of 1× TBST (TBS 0.2% Tween-20) 3 times. After last wash, liquid was removed and 50 μL per well of capture antibody (abcam ITK Y402-ab32507) was added to plates at a 1:1000 dilution in blocking buffer (above). Plates were sealed and rocked at room temperature for 2 hrs. Plates were then washed 3 times with 200 μL of 1× TBST. After last wash, liquid was removed and 45 μL of cell lysate (from above) was added to each plate. Plates were sealed and rocked overnight at 4° C. Next day, cell lysates were removed, and assay plates were washed 3 times with 200 μL of 1× TBST. After last wash, liquid was removed and detection antibody (CST ITK (2F12) #2380) was added at 50 μL per well at a dilution of 1:1000 in blocking buffer. Plates were sealed and rocked at room temperature for 2 hrs. Plates were washed 3 times with 200 uL of 1× TBST. After last wash, liquid was removed, MSD Mouse anti-Rabbit sulfo tag (R32AC-1) was diluted to 1:1000 in blocking buffer. 50 μL was added to each well on the plate. Plates were sealed and incubated at room temperature for 1 hr. Plates were washed 3 times with 200 μL of 1× TBST. After last wash, liquid was removed and 150 μL of 1× MSD Read Buffer T (R92PC) was then added to each well for ECL read out.

For reading ECL signal, plate is read on a Meso Scale Discovery (MSD) MESO Sector S 600 plate reader. Percent ITK in then calculated as described below.

Percent ITK remaining per sample was calculated as follows:

$$\% ITK remaining = 100 - [ \frac{Control ECL - Sample ECL}{Control ECL} ] \times 100$$

Using Graphpad Prism, % ITK remaining values were plotted as a function of compound concentration. To determine DC₅₀ and Dₘₐₓ values, resulting curves were fit to the Prism curve-fitting equation “log(inhibitor) vs response—Variable slope (four parameters)” (reported best fit value IC₅₀ used as DC₅₀).

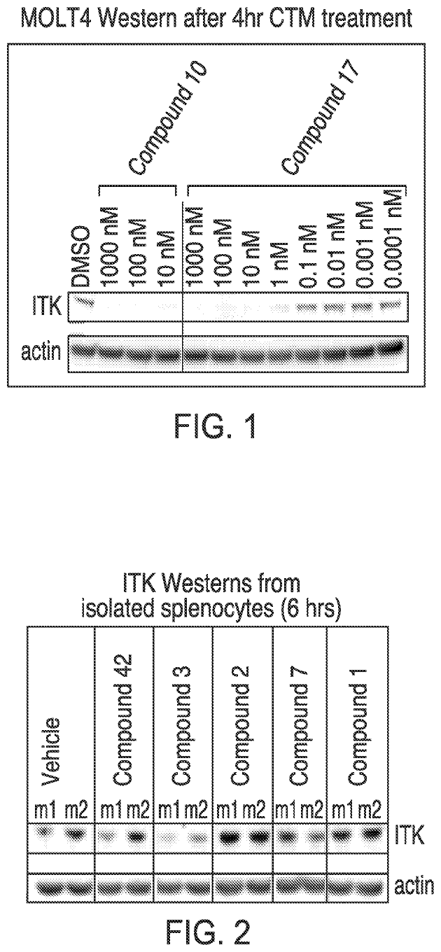

As shown in FIG. 1, compounds 10 and 17 degraded ITK with DC₅₀ values less than 10 nM. The Dmax values were 90-100% each.

### Biological Example 2—In Vivo Degradation After Oral Administration

### Mouse PD Assay for ITK Degradation

Compounds 1, 3, 7, and 42 were formulated and administered at 90 mg/kg to mice by oral gavage (PO). Blood was collected at defined time points (after 1, 2, and 6 hours) after compounds administration to determine PK parameters. The mean calculated concentrations are provided in Table 2 for compound 3. Concentration increased through 6 hours. Six hours later mice were sacrificed and splenocytes were isolated and used to prepare protein lysates. Total protein levels were determined in the lysates using the BCA assay according to standard methods. Equal amounts of protein were then analyzed by Western blot to determine the level of ITK expression in each sample. ITK band intensities were measured using Alpha View software and the percent of ITK remaining in each test animal was determined relative to the mean of ITK levels in vehicle control samples. As shown in FIG. 2, at least compounds 1, 3, and 7 degraded ITK in vivo at 6 hours.

TABLE 2

| Timepoint (h) | Concentration (μM) |
|---|---|
| 1 | 0.228 |
| 2 | 0.702 |
| 6 | 2.714 |

After 6 hours, splenocytes cells were harvested. ITK was evaluated by Western blotting. As shown in FIG. 2, at least compounds 1, 3, and 7 degraded ITK in vivo at 6 hours.

### Biological Example 3—In Vivo Degradation after Oral Administration

### Western Assay for ITK Degradation in Mouse Splenocytes

Compounds 43, 4, 5, 10, 11, 12, 44, 14, 3, and 13 were administered to mice orally. After 6 hours, splenocytes cells were harvested. ITK was evaluated by Western blotting. Media was removed and cell pellets were lysed in 100 uL lysis buffer (RIPA buffer (Fisher, PI89901), cOmplete Mini EDTA-free protease inhibitor (Sigma 11836170001), Protease Inhibitor Cocktail (Sigma, P2714), Phosphatase Inhibitor Cocktail 2 and 3 (Sigma, P5726 and P0044), Benzonase (Sigma, E1014)). Cells were lysed overnight at −20° C. Following thaw, cells were centrifuged for 10 min at 13000 rpm, then transferred to new tube. Protein levels were determined by BCA Assay performed according to manufacturer's protocol (EMID Millipore, cat. no. 71285-3). Samples were combined with (4×) LDS Sample Buffer and (10×) Reducing Agent and H₂O to equally load 20 ug protein per lane of a 26-well NuPAGE 4-12% Bis-Tris protein gel (1.0 mm, Thermo cat. no. NP0326). Samples were separated by running gels at constant 150 V in NuPAGE MES SDS Running Buffer. Following electrophoresis, proteins were transferred to nitrocellulose membranes using an iBlot Gel Transfer Device and iBlot Gel Transfer Stacks (Thermo cat. no. IB21001 and IB301001) and transfer method P3 (20V for 7 min). Membranes were blocked for 1 hr in 5% milk solution (TBS (0.2% Tween-20)). Following blocking, membranes were incubated with primary antibody (1:1000 CST ITK (2F12) #2380) overnight at 4° C. with gentle shaking. Blots were washed 2× in TBS (0.2% Tween-20), 30 min per wash. Following washes, blots were incubated in secondary HRP-conjugated antibody (Promega anti-Mouse IgG (H+L) HRP cat. no. W4021), 1:5000 in 5% milk solution (TBS (0.2% Tween-20)), for 1 hr at room temperature with gentle shaking. Blots were washed 2× in TBS (0.2% Tween-20), 30 min per wash. Blots were incubated with 1:1 mix of ECL reagents 1 & 2 (Amersham ECL Western Blotting Detection Reagent, cat. no. RPN2106) for 2 min at room temperature. Bands were visualized using a Protein Simple imager. Blots were then re-probed with a combination of anti-actin antibody (Sigma Monoclonal Mouse Anti-3-Actin (clone AC-15), cat. no. A5441) and secondary H₁RP-conjugated antibody (Promega anti-Mouse IgG (H+L) HRP, cat. no. W4021) and similar steps were taken for incubation, wash, detection and visualization steps as above. The data was analyzed using Alpha View software. The densitometric reading for each sample band was normalized to that of the corresponding actin band per lane. Approximate % ITK remaining per sample was calculated as follows:

$$\% ITK normalized to actin = [ \frac{ITK band - blot background}{actin band - blot background} ]$$

$$\% ITK remaining = [ \frac{Normalized ITK Sample}{Mean normalized ITK Control} ] \times 100$$

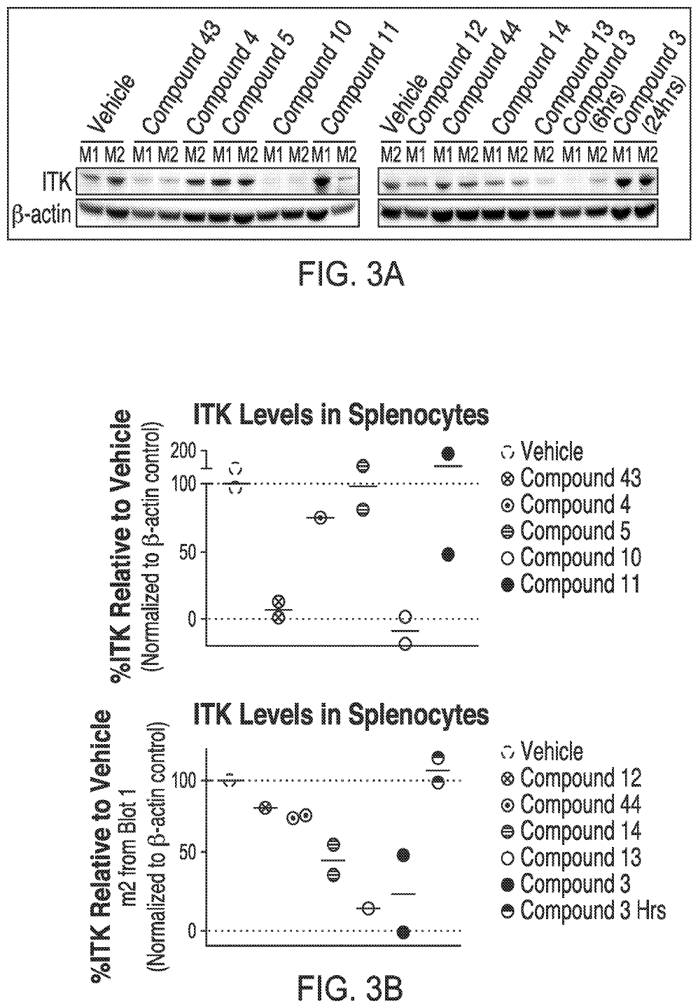

Once % ITK remaining has been calculated for each sample, groups are averaged together to show an means % ITK normalized to actin and relative to control. As shown in FIG. 3A, at least compounds 43, 4, 5, 10, 11, 12, 44, 14, 3, and 13 degraded ITK in vivo at 6 hours. Compound 3 degraded ITK in vivo at 6 hours and at 24 hours. As shown in FIG. 3B, the percent of ITK remaining decreased substantially compared to vehicle

### Biological Example 4—In Vivo Degradation after Oral Administration

### Mouse PK Analysis

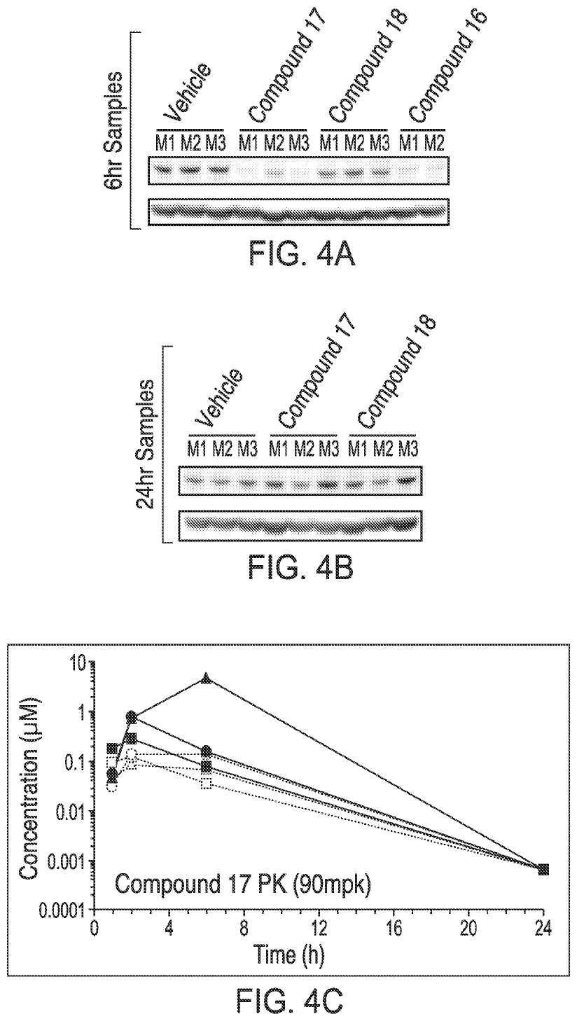

Compounds 16, 17, and 18 were administered to mice orally. After 6 hours or 24 hours, splenocytes cells were harvested. ITK was evaluated by Western blotting. Plasma concentrations were determined by LC/MS/MS. Plasma samples were protein precipitated by addition of 100 μl of acetonitrile containing 50 ng/ml of internal standard. The resulting mixture was vortexed and centrifuged at 4000 RPM for 5 minutes. An aliquot of the resultant supernatant (75 μl) was added to 75 μl of 0.1% formic acid in water to constitute the final sample for injection. Samples were injected on a Shimadzu Exion LC Binary Gradient AD Pump HLPC system connected to a Sciex QTRAP 6500+mass spectrometer. Five μl of sample was injected onto a Waters Acquity UPLC BEH C18 column 130A (2.1×30 mm, 1.7 m) at 40° C. utilizing a flow rate of 700 μl/minute. Mobile Phase A was 0.1% formic acid in water and mobile phase B was 0.1% formic acid in acetonitrile. A linear gradient of 15-95% B over 1.0 minute was used. The mass spectrometer was operated in positive ion electrospray mode with multiple reaction monitoring for maximum sensitivity. Sciex Analyst software (version 1.6.3) was used for LC/MS/MS instrument control and acquisition. Compound concentration was determined against a standard curve of internal standard versus compound peak area ratio. Noncompartmental PK parameters were determined using Phoenix 32 software (version 8.2.0.4383) from Certara. Plasma concentrations from the LC/MS/MS analyses, dose, route of administration, and desired units were utilized for PK parameter calculations. As shown in FIG. 4A, compounds 17, 18, and 16 degraded ITK in vivo at 6 hours, and compounds 17 and 18 degraded ITK at 24 hours. As shown in FIG. 4B, compounds 16 and 17 were detected in plasma through 6 and 24 hours, respectively. FIG. 4C provides the concentration over time of compound 17 following in vivo oral administration in the 6 hour and 24 hour assays.

### Noncompartmental PK Parameters

Cmax, Tmax and AUClast were all calculated using a WinNonLin Phoenix 64 v 8.2.0.4383. Using non-compartmental analysis, Tmax, Cmax and AUClast were determined as follows:
Tmax—Time of maximum observed concentration.
Cmax—Maximum observed concentration, occurring at time Tmax, as defined above.
AUClast—Area under the curve from the time of dosing to the time of the last measurable (positive) concentration (Tlast).

Results of biological assays are reported in Table 3A-3E.

TABLE 3A

| Compound | HiBiT | HiBiT |
|---|---|---|
| No. | DC₅₀ (nM) | D_{Max} (%) |
| 1 | 10.4 | 88 |
| 2 | 4.4 | 90 |
| 3 | 0.29 | 76 |
| 4 | 2.3 | 64 |
| 5 | 1.3 | 74 |
| 8 | 7.8 | 92 |
| 9 | 0.21 | 85 |
| 10 | 0.48 | 89 |
| 11 | 2 | 84 |
| 12 | 0.29 | 86 |
| 13 | 2.4 | 90 |
| 14 | 0.16 | 89 |
| 15 | 0.35 | 93 |
| 16 | 0.23 | 90 |
| 17 | <0.001 | 85 |
| 18 | 0.004 | 76 |
| 19 | 0.27 | 91 |
| 20 | NC | NC |
| 21 | 0.01 | 82 |
| 22 | 0.01 | 82 |
| 23 | <0.001 | 82 |
| 24 | 0.01 | 90 |
| 25 | <0.01 | 89 |
| 26 | <0.01 | 88 |
| 33 | 3.4 | 85 |
| 34 | 1.8 | 89 |
| 42 | 2.9 | 93 |
| 43 | 2.9 | 93 |
| 44 | 2.4 | 88 |
| 45 | 8.8 | 59 |
| 46 | 14 | 79 |
| 47 | 4.6 | 87 |
| 48 | 5.2 | 86 |
| 49 | 3.9 | 85 |
| 50 | 0.21 | 90 |
| 61 | <0.01 | 83 |
| 52 | <0.001 | 83 |
| 53 | 3.3 | 94 |
| 54 | 11 | 73 |
| 55 | 1.8 | 78 |
| 56 | 0.013 | 64 |
| 57 | <0.01 | 90 |
| 58 | 13.4 | 69 |
| 59 | 0.05 | 81 |
| 61 | 0.002 | 83 |
| 62 | 0.002 | 72 |
| 63 | 0.003 | 80 |
| 65 | 0.35 | 80 |
| 66 | 0.55 | 71 |
| 67 | 2.3 | 75 |
| 68 | 0.5 | 78 |
| 69 | — | 42 |
| 70 | — | 53 |
| 71 | 5.2 | 70 |
| 71a | — | 53 |
| 71b | 2 | 85 |
| 72 | 0.12 | 86 |
| 73 | 1.6 | 85 |
| 74 | 10.5 | 85 |
| 75 | 0.5 | 83 |
| 91 | 0.24 | 85 |
| 92 | <0.01 | 89 |
| 93 | 1.9 | 97 |
| 94 | 3.4 | 83 |
| 95 | 0.6 | 90 |
| 96 | 1.1 | 89 |
| 97 | 2.6 | 88 |
| 98 | 0.7 | 89 |
| 99 | 3.7 | 85 |
| 100 | 1.7 | 86 |
| 101 | 1.1 | 87 |
| 108 | 1.8 | 88 |
| 109 | 0.73 | 91 |
| 110 | 1.7 | 88 |
| 111 | 1.2 | 90 |
| 112 | 0.14 | 87 |
| 113 | 0.4 | 80 |
| 114 | 0.002 | 87 |
| 115 | 0.391 | 84 |
| 116 | 0.9 | 89 |
| 118 | 0.467 | 77 |
| 119 | 0.041 | 85 |
| 120 | 0.049 | 84 |
| 121 | 2.03 | 76 |
| 123 | 63.1 | 63 |
| 124 | 3.7 | 90 |
| 125 | 3.3 | 88 |
| 126 | 1.4 | 91 |
| 127 | 19.1 | 69 |
| 128 | 4.9 | 81 |
| 129 | 4.9 | 81 |
| 130 | 5.5 | 85 |
| 131 | 0.5 | 84 |
| 132 | 7 | 82 |
| 133 | 1.6 | 86 |
| 135 | <0.01 | 85 |
| 136 | 0.45 | 80 |
| 137 | 4.4 | 81 |
| 138 | 0.076 | 83 |
| 139 | 0.8 | 81 |
| 140 | 8.5 | 84 |
| 141 | 2.3 | 86 |
| 142 | 2.2 | 80 |
| 143 | 4.3 | 81 |
| 144 | 2.2 | 78 |
| 145 | 9.1 | 75 |
| 146 | 18.5 | 73 |
| 147 | 4.4 | 81 |
| 152 | 9 | 80 |
| 153 | 1.2 | 80 |
| 154 | 1.1 | 76 |
| 155 | 0.42 | 84 |
| 156 | 3.6 | 84 |
| 157 | 4.154 | 84 |
| 158 | 0.857 | 88 |
| 159 | 0.53 | 84 |
| 160 | 0.321 | 83 |
| 161 | 0.165 | 85 |
| 164 | <0.01 | 86 |
| 165 | <0.01 | 86 |
| 166 | 0.033 | 88 |
| 167 | 0.099 | 86 |
| 168 | 0.006 | 88 |
| 169 | 0.016 | 80 |
| 171 | 0.04 | 88 |
| 172 | 0.022 | 75 |
| 173 | 0.035 | 87 |
| 175 | 4.145 | 65 |
| 176 | 2 | 76 |
| 177 | 7 | 86 |
| 178 | 0.4 | 90 |
| 179 | 0.61 | 88 |
| 180 | 9 | 72 |
| 181 | 10.4 | 61 |
| 182 | 11.07 | 70 |
| 183 | 3.025 | 81 |
| 184 | 1 | 70 |
| 186 | 1.1 | 87 |
| 187 | 5.3 | 70 |
| 188 | 0.45 | 85 |
| 189 | 0.97 | 84 |
| 190 | 0.02 | 86 |
| 191 | 3.8 | 78 |
| 193 | 2 | 85 |
| 194 | 25 | 64 |
| 199 | 37 | 52 |
| 204 | 2.362 | 70 |
| 207 | 0.49 | 65 |
| 209 | 0.345 | 78 |
| 214 | 36 | 53 |
| 216 | 0.06 | 87 |
| 220 | 1.4 | 89 |
| 221 | 3.569 | 84 |
| 222 | 1.421 | 75 |
| 224 | 8.5 | 84 |
| 225 | 8.5 | 84 |
| 227 | 0.6 | 87 |

TABLE 3B

| Compound | MSD | MSD |
|---|---|---|
| No. | DC₅₀ (nM) | D_{Max} (%) |
| 1 | 7.0 | 87 |
| 2 | 1.1 | 84 |
| 6 | 0.0041 | 81 |
| 7 | 10 | 83 |
| 27 | 3.8 | 92 |
| 28 | 3.9 | 94 |
| 29 | 21 | 95 |
| 30 | 20 | 92 |
| 31 | 44 | 95 |
| 32 | 25 | 69 |
| 33 | 1.6 | 87 |
| 34 | 1 | 86 |
| 35 | 8 | 83 |
| 36 | 1.6 | 72 |
| 37 | 0.4 | 66 |
| 38 | 0.2 | 84 |
| 39 | 0.88 | 94 |
| 40 | NC | 46 |
| 41 | 0.57 | 86 |

TABLE 3C

| Compound | C_{Max} | T_{Max} | AUC Last |
|---|---|---|---|
| No. | (μM) | (h) | (h*μM) |
| 3 | 0.471 | 4 | 1.99 |
| 4 | 0.127 | 2 | 0.54 |
| 5 | 0.188 | 4 | 0.7 |
| 9 | 0.046 | 6 | 0.154 |
| 10 | 1.62 | 6 | 22.7 |
| 11 | 3.08 | 2 | 16.5 |
| 12 | 0.0803 | 1 | 0.198 |
| 13 | 0.251 | 4 | 0.927 |
| 15 | 0.743 | 4 | 3.14 |
| 16 | 0.585 | 6 | 1.53 |
| 17 | 0.87 | 2 | 1.25 |
| 18 | 0.694 | 3.3 | 1.98 |
| 19 | 0.379 | 6 | 1.56 |
| 20 | 0.609 | 1 | 1.69 |
| 21 | 2.43 | 1 | 7.07 |
| 22 | 2.43 | 1 | 7.07 |
| 43 | 0.72 | 6 | 2.34 |
| 44 | 0.027 | 2 | — |
| 46 | 2.13 | 4 | 9.38 |
| 47 | 7.11 | 6 | 36.5 |
| 48 | 2.44 | 6 | 8.42 |
| 51 | 0.72 | 6 | 2.34 |
| 58 | 0.061 | 1.5 | 0.18 |
| 61 | 0.023 | 1.5 | 0.08 |
| 65 | 0.405 | 6 | 1.38 |
| 66 | 0.526 | 4 | 1.74 |
| 69 | 0.283 | 3.5 | 1.26 |
| 71b | 0.223 | 1.5 | 0.78 |
| 72 | 0.009 | 4 | 0.02 |

TABLE 3D

| | Cal. | Cal. | Cal. |
|---|---|---|---|
| | Conc. | Conc. | Conc. |
| | Total | Total | Total |
| Compound | (μM) | (μM) | (μM) |
| No. | 1 h | 2 h | 6 h |
| 1 | 1.6 | 1.7 | 0.7 |
| 2 | 0.041 | 0.039 | 0.059 |
| 7 | 0.20 | 0.29 | 0.45 |
| 37 | — | 0.24 | 0.17 |
| 34 | — | BQL | BQL |
| 35 | — | 0.155 | 0.053 |
| 38 | — | 0.0046 | 0.0024 |
| 40 | — | BQL | 0.009 |

TABLE 3E

| | ITK (%) |
|---|---|
| Compound | 90 mpk |
| No. | PO, 6 h |
| 1 | 153 |
| 2 | 49.3 |
| 3 | 24 |
| 4 | 75 |
| 5 | 97 |
| 6 | 105 |
| 7 | 100 |
| 9 | 49.3 |
| 10 | 11 |
| 11 | 116 |
| 12 | 82 |
| 13 | 15 |
| 14 | 47 |
| 15 | 18.3 |
| 16 | 77 |
| 17 | 20 |
| 18 | 14 |
| 19 | 8 |
| 20 | 66 |
| 21 | 76 |
| 22 | 76 |
| 33 | 67 |
| 34 | 108 |
| 35 | 108 |
| 42 | 82 |
| 43 | 7 |
| 44 | 75 |
| 46 | 102 |
| 47 | 11 |
| 48 | 35 |
| 49 | 129 |
| 50 | 113 |
| 65 | 58 |
| 66 | 42.7 |
| 67 | 190 |
| 68 | 240 |
| 69 | 140 |
| 71b | 30 |
| 72 | 116 |
| 73 | 92 |
| 75 | 137 |

### OTHER EMBODIMENTS

It is to be understood that the foregoing description is intended to illustrate and not limit the scope of this disclosure, which is defined by the scope of the appended claims. Other aspects, advantages, and modifications are within the scope of the following claims.

What is claimed is:

1. A compound of Formula (I): 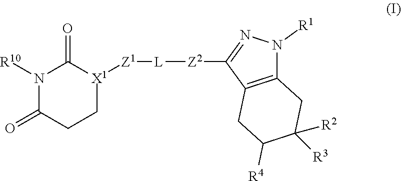
wherein
X¹ is C—H or nitrogen;
Z¹ is selected from the group consisting of 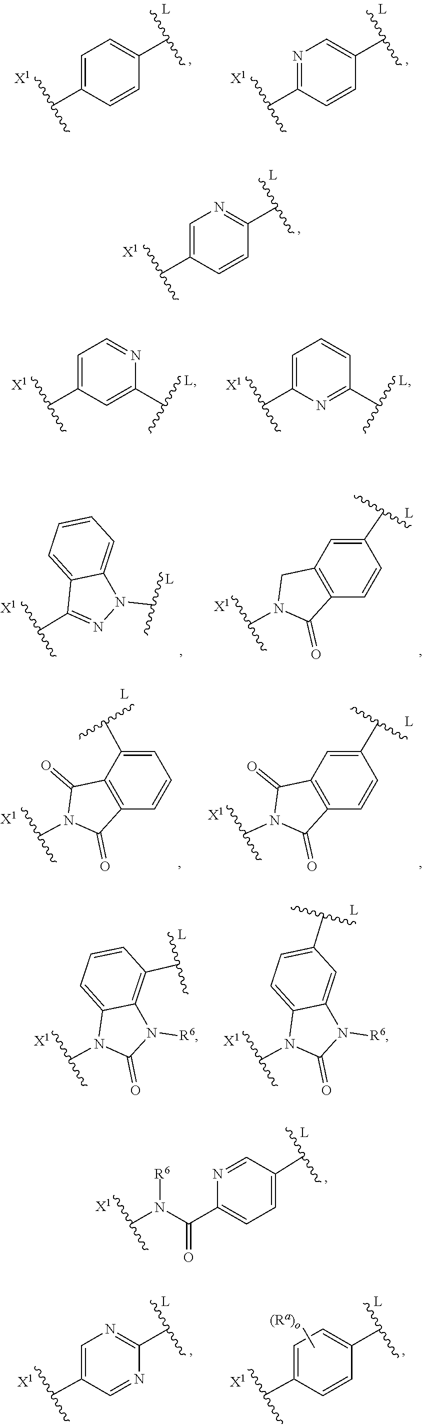 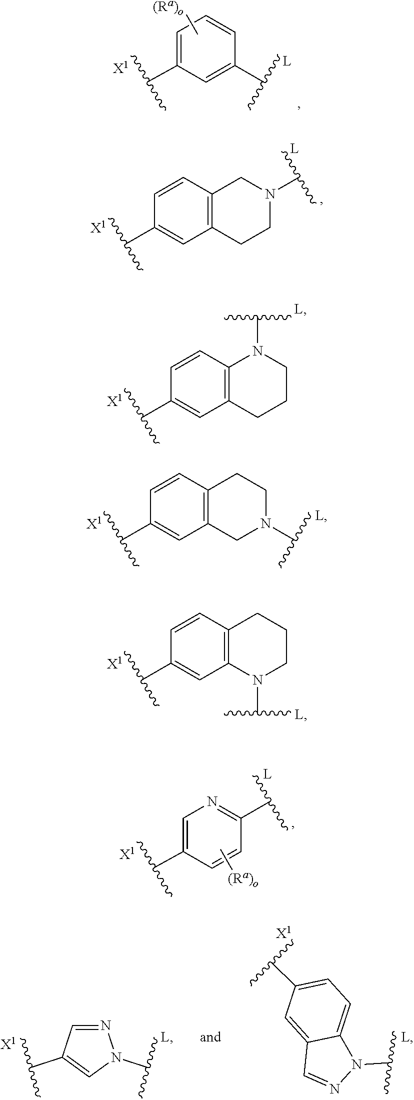
wherein [symbol] designates attachment to X¹; and [symbol] designates attachment to L;
L is a linker according to
-L¹-L²-L³-L⁴-L⁵-L⁶-L⁷- wherein
-L¹- is absent, —N(R²¹)—; C(R²²)—; substituted or unsubstituted C₁₋₈ alkylene; substituted or unsubstituted C₂₋₈ alkynylene; Q¹; or Q²;
each -L²-, -L³-, -L⁴-, and -L⁵- is independently, absent; —N(R²¹)—; C(R²²)—; —C(O)—; —O—(CH₂—CH₂—O)₁₋₈; substituted or unsubstituted C₁₋₈ alkylene; substituted or unsubstituted C₂₋₈ alkynylene; Q¹; Q²; or Q³;
each -L⁶- and -L⁷-, is independently, absent; —N(R²¹)—; —C(R²²)—; —C(O)—; —C(O)—N(R²¹-—N(R²¹)—C(O); or —C(R²²)—C(O)—N(R²¹);
each Q¹ is a three- to eight-membered heterocycloalkylene comprising at least one nitrogen, wherein the heterocycloalkylene is optionally subsituted;
each Q² is a five- to thirteen-membered spiro bicyclic heterocycloalkylene comprising at least one nitrogen, wherein the heterocycloalkylene is optionally subsituted;
each Q³ is a three- to six-membered cycloalkylene, wherein the cycloalkylene is optionally subsituted;
Z² is 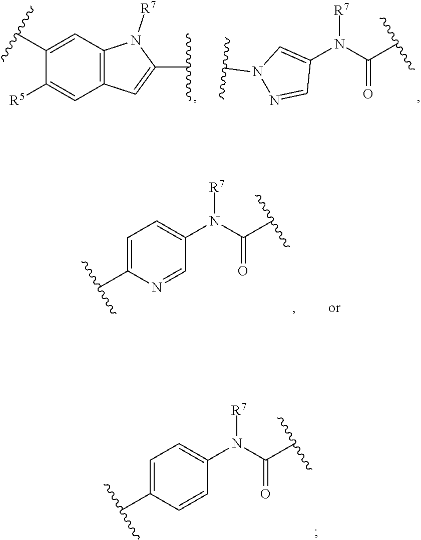
R¹ is hydrogen C₁₋₃ alkyl;
R² is hydrogen, substituted or unsubsituted C₁₋₅ alkyl, or substituted or unsubsituted C₁₋₅ alkoxy;
R³ is methyl, or methylene bound to R⁴ to form a substituted or unsubsituted cyclopropyl;
R⁴ is hydrogen, or methylene bound to R³ to form the substituted or unsubstituted cyclopropyl;
R⁵ is hydrogen, halogen, or —C₁-C₃ alkyl;
each R⁶ is hydrogen, halogen, or —C₁-C₃ alkyl;
each R⁷ is hydrogen, halogen, or —C₁-C₃ alkyl;
R¹⁰ is hydrogen or substituted or unsubstituted C₁₋₈ alkyl;
each R²¹ is hydrogen or methyl; and
each R²² is hydrogen, methyl, aryl, or heteroaryl, wherein the aryl and heteroaryl are optionally subsituted;
R^{a} is halogen, —C₁-C₃ alkyl, or —C₁-C₃ alkoxy;
o is 1, 2, 3, or 4; or
a stereoisomer, and/or pharmaceutical salt(s) thereof.

2. The compound of claim 1, wherein Z¹ is selected from the group consisting of 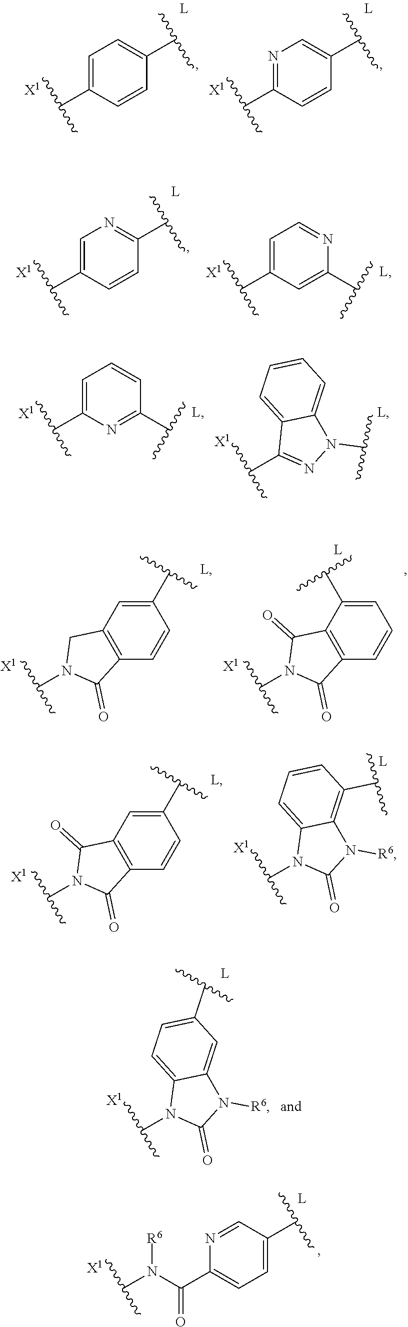
wherein [symbol] designates attachment to X¹; and [symbol] designates attachment to L;
L is a linker according to
-L¹-L²-L³-L⁴-L⁵-L⁶-L⁷- wherein
-L¹- is absent, —N(R²¹)—; C(R²²)—; C₁₋₈ alkylene; C₂₋₈ alkynylene; Q¹; or Q²;
each -L²-, -L³-, -L⁴-, and -LS- is independently, absent; —N(R²¹)—; C(R²²)—; —C(O)—; —O—; —(CH₂—CH₂—O)₁₋₈—; C₁₋₈ alkylene; C₂₋₈ alkynylene; Q¹; Q²; or Q³;
each -L⁶- and -L⁷-, is independently, absent; —N(R²¹)—; —C(R²²)—; —C(O)—; —C(O)—N(R²¹-—N(R²¹)—C(O); or —C(R²²)—C(O)—N(R²¹);
each Q¹ is a three- to seven-membered heterocycloalkylene comprising at least one nitrogen;
each Q² is a five- to thirteen-membered spiro bicyclic heterocycloalkylene comprising at least one nitrogen;
each Q³ is a three- to six-membered cycloalkylene;
Z² is 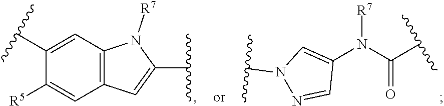
R¹ is hydrogen or methyl;
R² is methyl;
R³ is methyl, or methylene bound to R⁴ to form a substituted cyclopropyl;
R⁴ is hydrogen, or methylene bound to R³ to form the substituted cyclopropyl;
R⁵ is hydrogen or halogen;
each R⁶ is hydrogen or methyl;
each R⁷ is hydrogen or methyl;
R¹⁰ is hydrogen, or C₁₋₈alkyl;
each R²¹ is hydrogen or methyl; and
each R²² is hydrogen, methyl, aryl, or heteroaryl; or
a stereoisomer, and/or pharmaceutical salt(s) thereof.

3. The compound of claim 1, wherein Z¹ is selected from the group consisting of 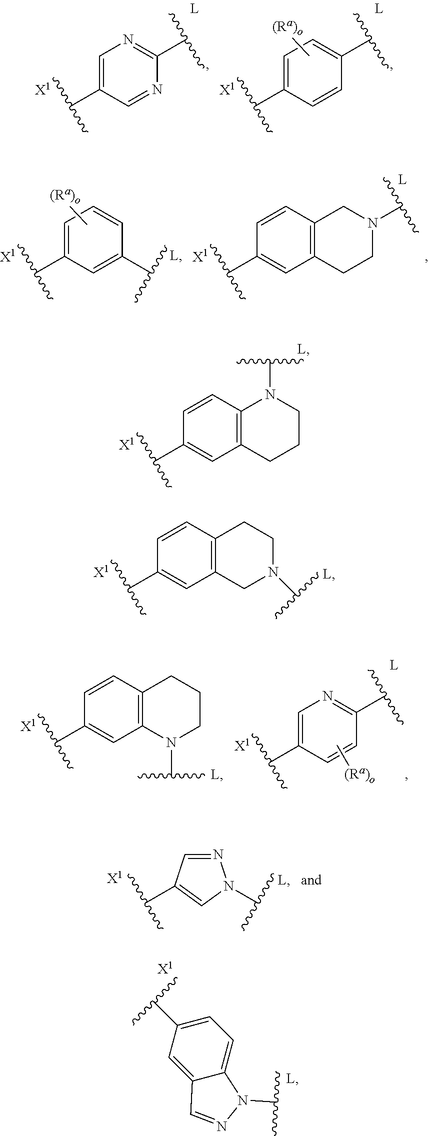
wherein [symbol] designates attachment to X¹; and [symbol] designates attachment to L;
L is a linker according to
-L¹-L²-L³-L⁴-L⁵-L⁶-L⁷- wherein
-L¹- is absent, —N(R²¹)—; C(R²²)—; substituted or unsubstituted C₁₋₈ alkylene; substituted or unsubstituted C₂₋₈ alkynylene; Q¹; or Q²;
each -L²-, -L³-, -L⁴-, and -L⁵- is independently, absent; —N(R²¹)—; C(R²²)—; —C(O)—; —O—(CH₂—CH₂—O)₁₋₈; substituted or unsubstituted C₁₋₈ alkylene; substituted or unsubstituted C₂₋₈ alkynylene; Q¹; Q²; or Q³;
each -L⁶- and -L⁷-, is independently, absent; —N(R²¹)—; —C(R²²)—; —C(O)—; —C(O)—N(R²¹-—N(R²¹)—C(O); or —C(R²²)—C(O)—N(R²¹);
each Q¹ is a three- to seven-membered heterocycloalkylene comprising at least one nitrogen, wherein the heterocycloalkylene is optionally subsituted;
each Q² is a five- to thirteen-membered spiro bicyclic heterocycloalkylene comprising at least one nitrogen, wherein the heterocycloalkylene is optionally subsituted;
each Q³ is a three- to six-membered cycloalkylene, wherein the cycloalkylene is optionally subsituted;
Z² is 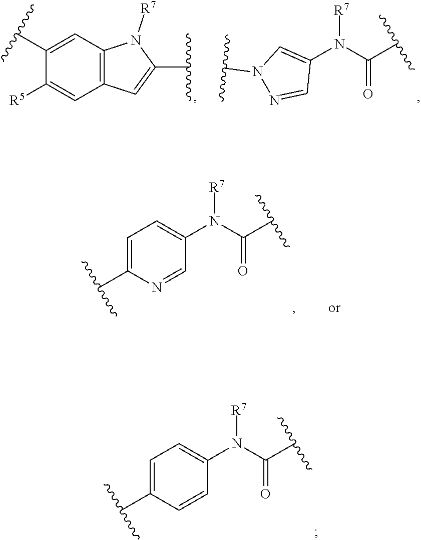
R¹ is hydrogen, C₁₋₃ alkyl;
R² is hydrogen, substituted or unsubsituted C₁₋₅ alkyl, or substituted or unsubsituted C₁₋₅ alkoxy;
R³ is methyl, or methylene bound to R⁴ to form a substituted or unsubsituted cyclopropyl;
R⁴ is hydrogen, or methylene bound to R³ to form the substituted or unsubstituted cyclopropyl;
R⁵ is hydrogen, halogen, or —C₁-C₃ alkyl;
each R⁶ is hydrogen, halogen, or —C₁-C₃ alkyl;
each R⁷ is hydrogen, halogen, or —C₁-C₃ alkyl;
R¹⁰ is hydrogen or substituted or unsubstituted C₁₋₈ alkyl;
each R²¹ is hydrogen or methyl; and
each R²² is hydrogen, methyl, aryl, or heteroaryl, wherein the aryl and heteroaryl are optionally subsituted;
R^{a} is halogen, —C₁-C₃ alkyl, or —C₁-C₃ alkoxy;
o is 1, 2, or 3; or
a stereoisomer, and/or pharmaceutical salt(s) thereof.

4. The compound of any of the previous claims, wherein when R³ and R⁴ form the substituted cyclopropyl, then the cyclopropyl is substituted with difluoro.

5. The compound of any of the previous claims, wherein Z² is 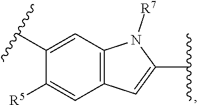
wherein R⁵ is hydrogen or —F.

6. The compound of any of the previous claims, wherein Z² is

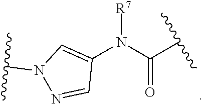

7. The compound of claim 1 or 3, wherein Z² is

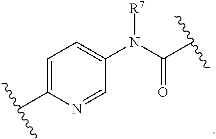

8. The compound of claim 1 or 3, wherein Z² is

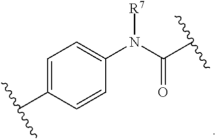

9. The compound of any of the previous claims wherein R² is methyl; R³ is methyl; and R⁴ is hydrogen.

10. The compound of any one of claim 1 or 3-8, wherein R² is hydrogen, methyl, —CH₂OH, or —CH₂OCH₃; R³ is methyl; and R⁴ is hydrogen.

11. The compound of any of the previous claims wherein R³ is methylene bound to R⁴ to form a subsituted cyclopropyl substituted with difluoro.

12. The compound of claim 1 and 3, wherein R³ is methylene bound to R⁴ to form an unsubsituted cyclopropyl.

13. The compound of claim 1 and 3, wherein R^{a} is —F, —CH₃, or —OCH₃.

14. The compound of claim 13, wherein o is 1.

15. The compound of any of the previous claims wherein R² is hydrogen; and R³ and R⁴ form difluorocyclopropane.

16. The compound of any of the previous claims, wherein X¹ is C; and R¹⁰ is hydrogen.

17. The compound of any of the previous claims, wherein X¹ is C; and R¹⁰ is methyl.

18. The compound of any of the previous claims, wherein X¹ is N; and R¹⁰ is hydrogen.

19. The compound of any of the previous claims, wherein Z¹ is selected from the group consisting of 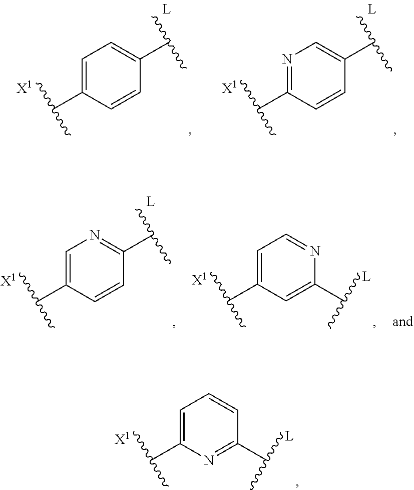
wherein [symbol] designates attachment to X¹; and [symbol] designates attachment to L.

20. The compound of any of the previous claims, wherein Z¹ is 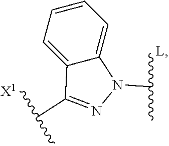
wherein [symbol] designates attachment to X¹; and [symbol] designates attachment to L.

21. The compound of any of the previous claims, wherein Z¹ is selected from the group consisting of 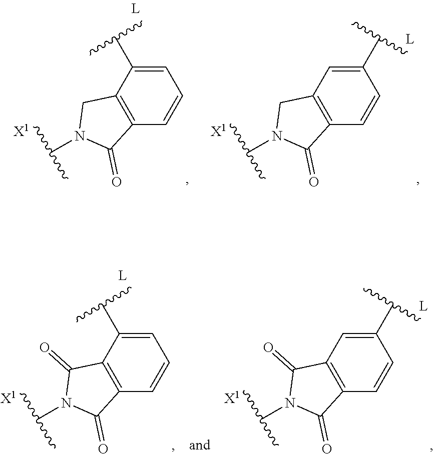
wherein [symbol] designates attachment to X¹; and [symbol] designates attachment to L.

22. The compound of any of the previous claims, wherein Z¹ is selected from the group consisting of 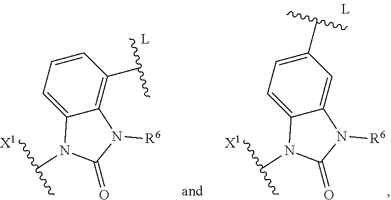
wherein [symbol] designates attachment to X¹; and [symbol] designates attachment to L.

23. The compound of any of the previous claims, wherein Z¹ is 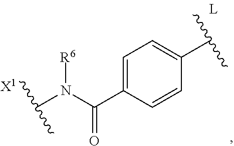
wherein [symbol] designates attachment to X¹; and [symbol] designates attachment to L.

24. The compound of claim 1 and 3, wherein Z¹ is selected from the group consisting of 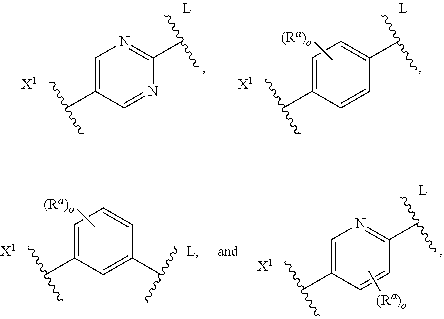
wherein [symbol] designates attachment to X¹; and [symbol] designates attachment to L.

25. The compound of claim 1 and 3, wherein Z¹ is selected from the group consisting of 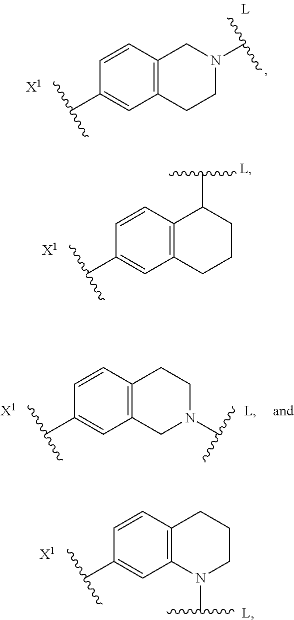
wherein [symbol] designates attachment to X¹; and [symbol] designates attachment to L.

26. The compound of claim 1 and 3, wherein Z¹ is 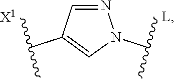
wherein [symbol] designates attachment to X¹; and [symbol] designates attachment to L.

27. The compound of claim 1 and 3, wherein Z¹ is 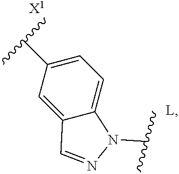
wherein [symbol] designates attachment to X¹; and [symbol] designates attachment to L.

28. The compound of any of the previous claims, wherein L comprises at least one -Q¹-according to 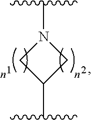
wherein n¹ is one, or two, and n² is one, or two, wherein -Q¹- is unsubsituted or mono- or di-subsituted.

29. The compound of claim 27, wherein -Q¹- is unsubstituted.

30. The compound of claim 27, wherein -Q¹- is mono- or di-subsituted with group independently selected from halogen and —C₁-C₃ alkyl.

31. The compound of claim 29, wherein -Q¹- is mono- or di-subsituted with group independently selected from —F, —CH₃, and —CH₂CH₃.

32. The compound of any of the previous claims wherein L is selected from:
a. -Q¹-N(Me)-CH₂-Q¹-C(O)—;
b. —N(Me)-Q¹-CH₂-Q¹-C(O)—;
c. -Q²-CH₂-Q¹-C(O)—;
d. -Q¹-CH₂-Q¹-C(O)—;
e. -Q¹-Q¹-C(O)—;
f. -Q¹-CH₂—N(Me)-Q¹-C(O)—;
g. -Q¹-CH₂-Q¹-CH₂—C(O)—N(Me)-;
h. -Q¹-N(Me)-CH₂-Q¹-C(O)—;
i. -Q¹-CH₂-Q¹-;
j. -Q¹-CH₂-Q²-;
k. -Q¹-CH₂—CH₂-Q¹-;
l. -Q¹-CH₂—CH₂-Q²-;
m. -Q¹-C(O)-Q¹-;
n. -Q¹-C(O)-Q²-;
o. -Q¹-CH₂-Q¹-N(Me)-C(O)—;
p. —CH₂—CH₂—CH₂—CH₂-Q¹-C(O)—;
q. -Q¹-CH₂-Q¹-C(O)—;
r. -Q¹-C(O)—;
S. -Q¹-C(O)-Q¹-C(C₆H₅)—;
t. —C≡CCH₂-Q¹-C(O)—;
U. -Q¹-C(O)-Q²-;
v. -Q¹-CH₂—CH₂-Q²-;
W. -Q¹-CH₂-Q¹-N—C(O)—;
x. —CH₂—CH₂—CH₂-Q¹-C(O)—;
y. -Q¹-CH₂-Q¹-C(Me)-C(O)—N(Me)-;
z. -Q¹-Q¹-C(O)—;
aa. —CH₂-Q¹-;
bb. -Q¹-C(O)-Q¹-CH₂—;
cc. —N(H)—(CH₂)₅—C(O)-Q¹-C(C₆H₅)—;
dd. —N(H)—(CH₂)₂—O—(CH₂)₂—C(O)-Q¹-C(C₆H₅)—;
ee. -Q¹-(CH₂)₃—C(O)-Q¹-C(C₆H₅)—;
ff. -Q²-C(O)-Q¹-C(C₆H₅)—;
gg. -Q²-CH₂—C(O)-Q¹-C(C₆H)—;
hh. -Q²-(CH₂)₃—C(O)-Q¹-C(C₆H₅)—;
ii. -Q²-(CH₂)₂—C(O)-Q¹-C(C₆H₅)—;
jj. —(CH₂)₆-Q¹-C(C₆H₅)—;
kk. -Q¹-Q¹-C(O)-Q¹-C(C₆H₅)—;
ll. -Q¹-CH₂—C(O)-Q¹-C(C₆H₅)—;
mm. -Q¹-(CH₂)₂—C(O)-Q¹-C(C₆H₅)—;
nn. -Q¹-(CH₂)₃—C(O)-Q¹-C(C₆H₅)—;
oo. —(CH₂)₃—C(O)-Q¹-C(C₆H₅)—;
pp. —(CH₂)₄—C(O)-Q¹-C(C₆H₅)—;
qq. —(CH₂)₅—C(O)-Q¹-C(C₆H₅)—;
rr. —(CH₂)₆—C(O)-Q¹-C(C₆H₅)—;
ss. —(CH₂)₃-Q¹-CH₂—C(O)-Q¹-C(C₆H₅)—;
tt. —(CH₂)₆-Q¹-C(C₆H₅)—;
uu. —(CH₂)₆-Q¹-C(thiazolyl)-;
vv. —(CH₂)₃—O-Q³-C(O)-Q¹-C(C₆H₅)—;
ww. —(CH₂)₃—O—(CH₂)₂—C(O)-Q¹-C(C₆H₅)—;
xx. —(CH₂)₃—O—(CH₂)₂—C(O)-Q¹-C(thiazolyl)-;
yy. —(CH₂)₃—O—(CH₂)₂—C(O)-Q¹-C(pyrid-2-yl)-;
zz. —(CH₂)₄-Q¹-C(C₆H₅)—;
aaa. —(CH₂)₅-Q¹-C(C₆H₅)—;
bbb. —(CH₂)₆-Q¹-C(C₆H₅)—;
ccc. —(CH₂)₆-Q¹-C(thiazolyl)-;
ddd. —(CH₂)₆-Q¹-C(pyrid-2-yl)-;
eee. —(CH₂)₇-Q¹-C(C₆H₅)—;
fff. —(CH₂)₇-Q¹-C(Me)-C(O)—N(Me)-;
ggg. —N(H)—(CH₂)₂—O—(CH₂)₂-Q¹-C(Me)-C(O)—N(Me)-;
hhh. —(CH₂)₃—O—(CH₂)₂—C(O)-Q¹-C(Me)-C(O)—N(Me)-;
iii. —N(H)—(CH₂)₂—O—(CH₂)₂-Q¹-C(C₆H₅)—;
jjj. —N(H)—(CH₂)₂—O—(CH₂)₂—C(O)-Q¹-C(C₆H₅)—;
kkk. —N(H)—(CH₂)₂—[O—(CH₂)₂]₂—C(O)-Q¹-C(C₆H₅)—;
lll. —N(H)—(CH₂)₂—[O—(CH₂)₂]₃—C(O)-Q¹-C(C₆H₅)—;
mmm. —N(H)—(CH₂)₂—[O—(CH₂)₂]₄—C(O)-Q¹-C(C₆H₅)—;
nnn. —N(H)—(CH₂)₂—[O—(CH₂)₂]₅—C(O)-Q¹-C(C₆H₅)—;
ooo. —N(H)—(CH₂)₂—[O—(CH₂)_{2]6}—C(O)-Q¹-C(C₆H₅)—;
ppp. —N(H)—(CH₂)₂—[O—(CH₂)₂]₇—C(O)-Q¹-C(C₆H₅)—; and
qqq. —N(H)—(CH₂)₂—[O—(CH₂)₂]s-C(O)-Q¹-C(C₆H₅)—;
rrr —N(H)-Q³-O—(CH₂)₂—CH₂—;
sss —N(H)—(CH₂)₃-Q¹-(CH₂)₂—;
ttt —C(O)—N(H)—[(CH₂)₃—O]₃—(CH₂)₂—NH—;
uuu —C(O)—N(H)—[(CH₂)₃-0]₃-(CH₂)₂—;
vvv -Q¹-C(O)—[(CH₂)₂—O]₃—(CH₂)₂—NH—;
www -Q¹-(CH₂)₃—O—CH₂—;
xxx -Q¹-C(O)—(C₆H₆)—CH₂—;
yyy -Q¹-(2-pyridyl)-O—CH₂—;
zzz —N(H)-Q³-X-(2-pyridyl)-O—;
aaaa —N(H)-Q³-X-(4-pyridyl)-; and
bbbb —N(H)—(CH₂)₂-Q³-X-(2-pyridyl)-O—CH₂—,
cccc —CH═C—(CH₂)₂Q¹-; and
dddd -Q¹-;
eeee -Q¹-CH₂—C(O)—N(CH₃)—;
ffff -Q¹-CH₂-Q¹-C(O)—;
gggg -Q¹-CH₂—CH₂-Q¹-C(O)—;
hhhh -Q¹-N(CH₃)—C(O)-Q¹-C(O)—;
iiii —CH₂-Q¹-C(O)—;
jjjj -Q¹-CH₂-Q¹-;
kkkk -Q¹-CH₂—C(O)-Q¹-;
lll —O-Q¹-C(O)-Q¹-;
mmmm —C(O)-Q¹-;
nnnn -Q¹-C(O)-Q¹-;
oooo —CH₂—C(O)-Q¹-;
pppp —C(O)-Q¹-CH₂-Q¹-;
qqqq —C(O)-Q¹-CH₂-Q¹-C(O)—;
rrrr —CH₂-Q¹-CH₂-Q¹-C(O)—;
ssss —C(O)-Q¹-CH₂-Q¹-CH₂—;
tttt -Q¹-CH₂-Q¹-CH₂—;
uuuu -Q¹-C(O)-Q¹-CH₂—;
vvvv —CH₂-Q¹-, wherein X is oxygen or sulfur.

33. The compound of any of the previous claims, wherein L comprises at least one -Q¹-selected from the group consisting of 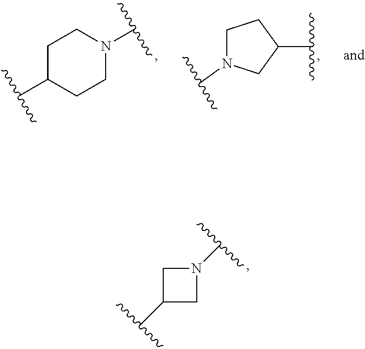 [SMILES: *C1CCN(*)CC1 ; *C1CCN(*)C1 ; *C1CN(*)C1]
wherein -Q¹ is optionally mono- or di-substituted with group independently selected from —F, —CH₃, and —CH₂CH₃.

34. The compound of any of the previous claims, wherein L comprises at least one -Q²-according to 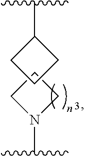
wherein n³ is one, or two.

35. The compound of any of the previous claims, wherein L comprises at least one -Q²-according to

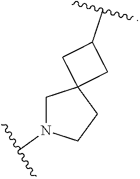 [SMILES: *C1CC2(CCN(*)C2)C1]

36. The compound of any of the previous claims, wherein L comprises at least one -Q²-according to 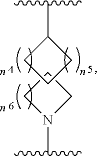
wherein n⁴ is one, or two, n⁵ is one, or two, and n⁶ is one, or two.

37. The compound of any of the previous claims, wherein L comprises at least one -Q²-according to

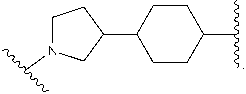 [SMILES: *C1CCC(C2CCN(*)C2)CC1]

38. The compound of any of the previous claims, wherein L comprises at least one -Q²-according to 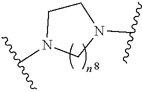
wherein n⁸ is one, or two, wherein -Q²- is optionally mono- or di-substituted with group independently selected from —F, —CH₃, and —CH₂CH₃.

39. The compound of any of the previous claims, wherein L comprises at least one -Q²-according to 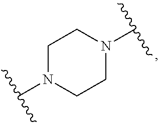 [SMILES: *N1CCN(*)CC1]
wherein -Q²- is optionally mono- or di-substituted with group independently selected from —F, —CH₃, and —CH₂CH₃.

40. The compound of any of the previous claims, wherein L comprises at least one -Q²-according to 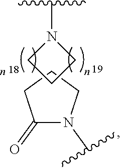
wherein n¹⁸, and n¹⁹ is two, or piperidinyl; or when n¹⁸ is two, then n¹⁹ is three, or azepanyl; or when n¹⁸ is three, then n¹⁹ is two, or azepanyl.

41. The compound of any of the previous claims, wherein L comprises at least one -Q²-according to

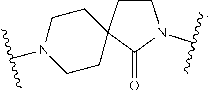 [SMILES: *N1CCC2(CC1)CCN(*)C2=O]

42. The compound of any of the previous claims, wherein L comprises at least one -Q²-according to 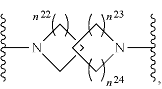
wherein n²² is zero to two; n²³ is zero to two, and n²⁴ is one, or two.

43. The compound of the previous claim, wherein when n²² is two, then n²³ and n²⁴ is one; or
when n²² is two, then each n²³ and n²⁴ is two.

44. The compound of any of the previous claims, wherein L comprises at least one -Q²-according to

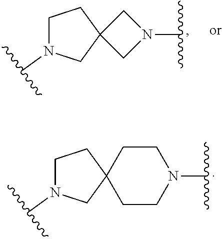 [SMILES: *N1CCC2(C1)CN(*)C2 ; *N1CCC2(CC1)CCN(*)C2]

45. The compound of any of the previous claims, wherein L comprises at least one -Q²-according to

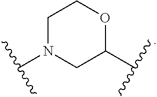 [SMILES: *C1CN(*)CCO1]

46. The compound of any of the previous claims, wherein L comprises at least one -Q³-according to 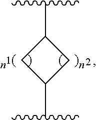
wherein n¹ is one, or two, and n² is one, or two.

47. The compound of any of the previous claims, wherein L comprises at least one -Q³-selected from the group consisting of

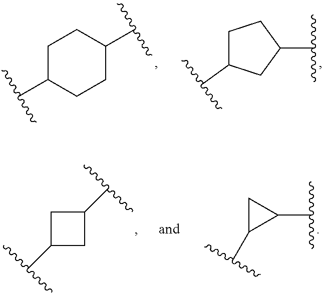 [SMILES: *C1CCC(*)CC1 ; *C1CCC(*)C1 ; *C1CC(*)C1 ; *C1CC1*]

48. The compound of any of the preceding claims, wherein the linker group is —C(O)—.

49. The compound of any of the preceding claims, wherein the linker group is selected from: 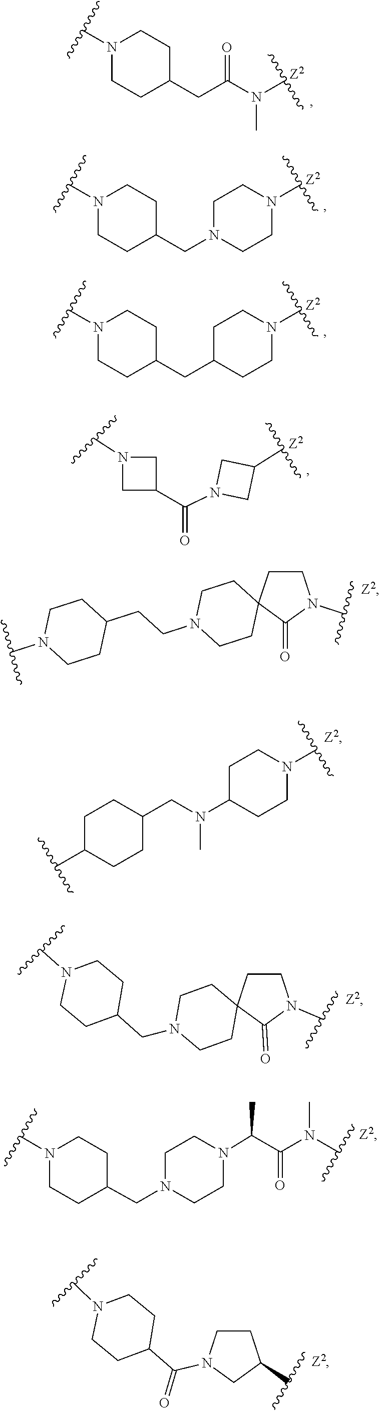 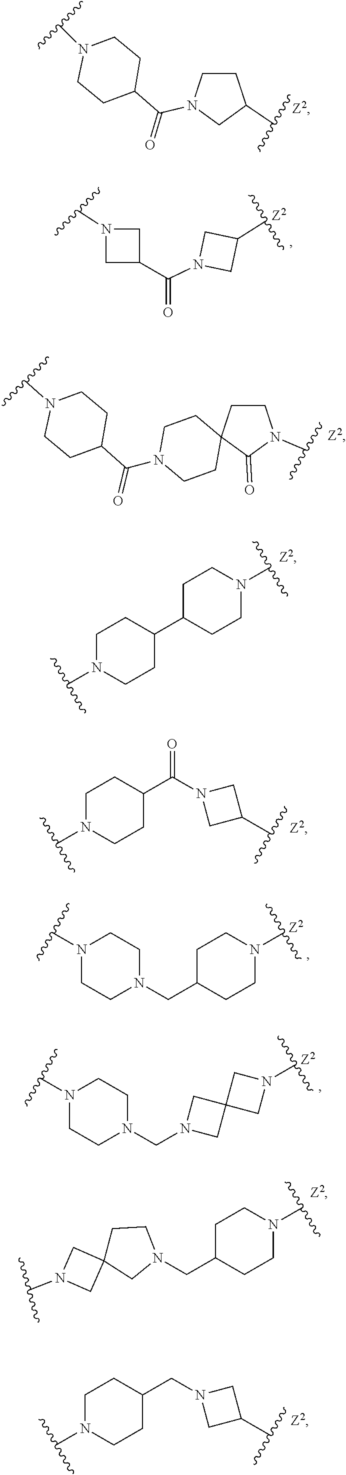 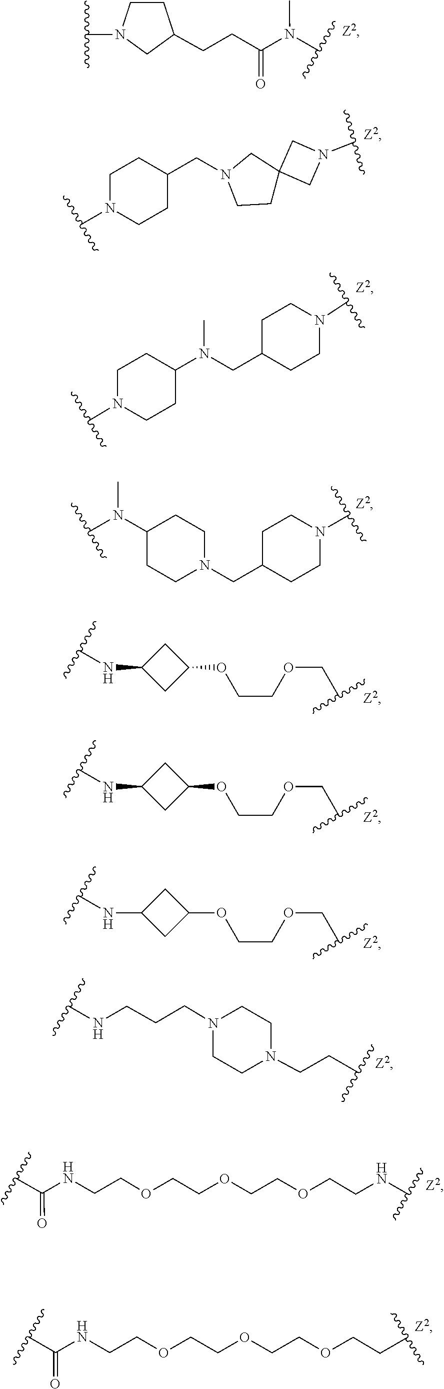 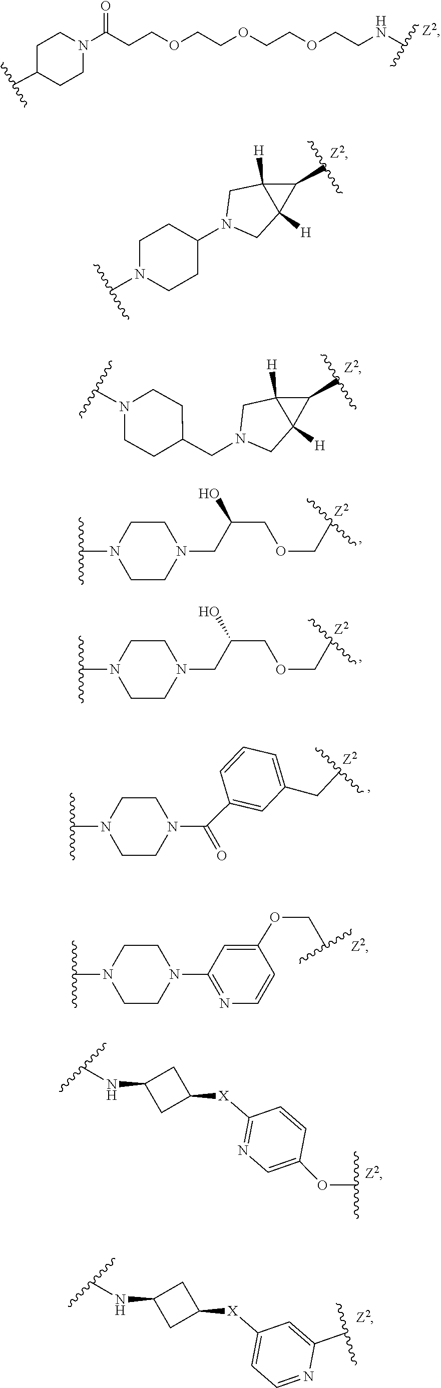 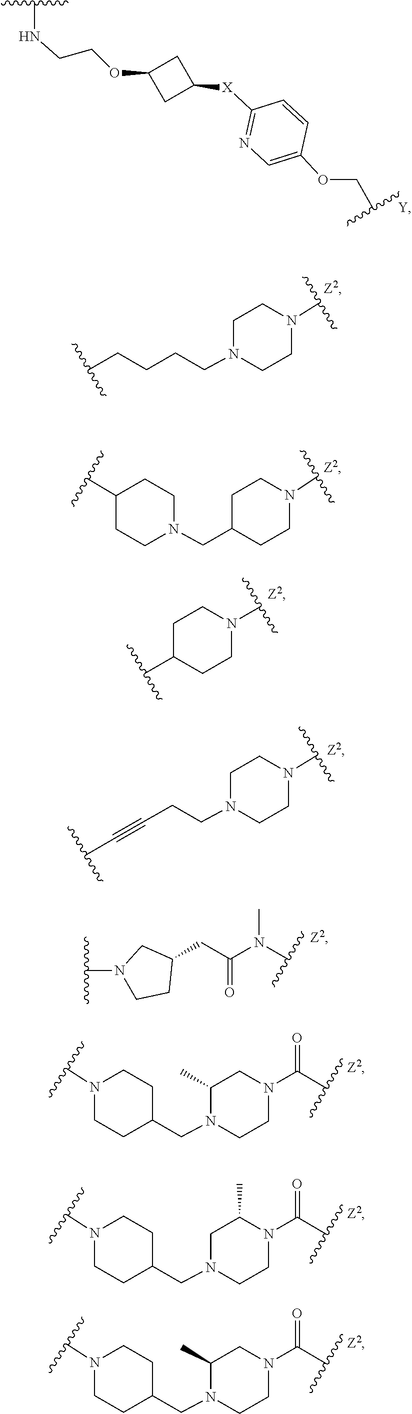 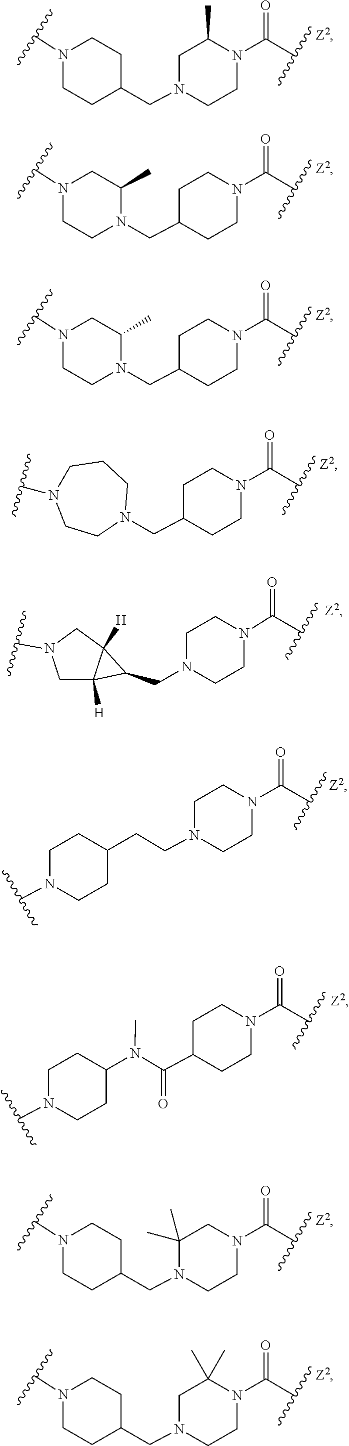 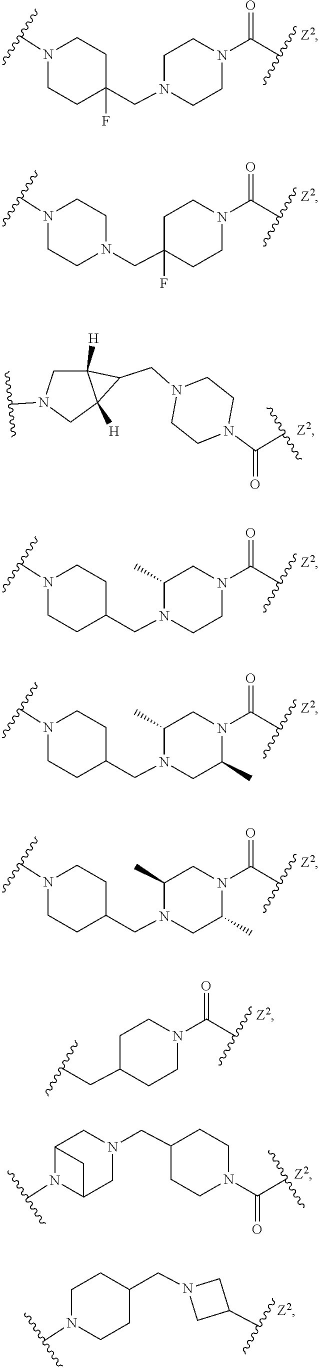 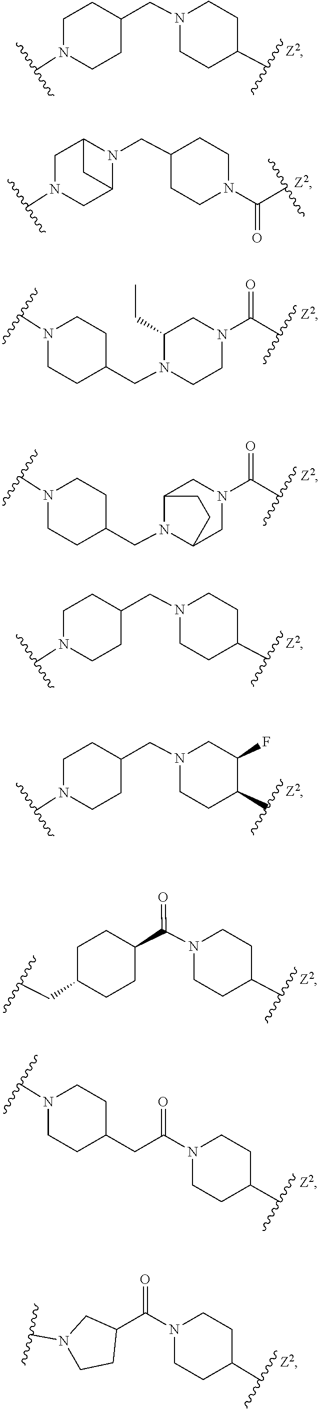 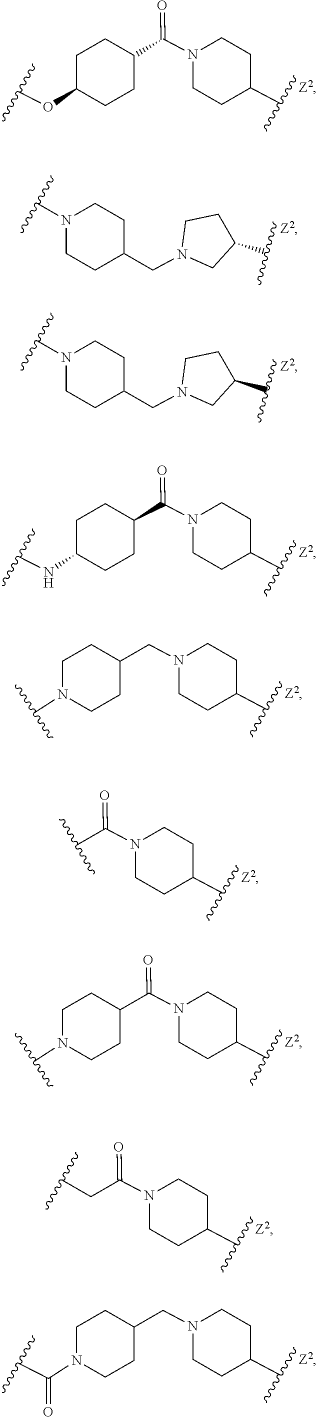 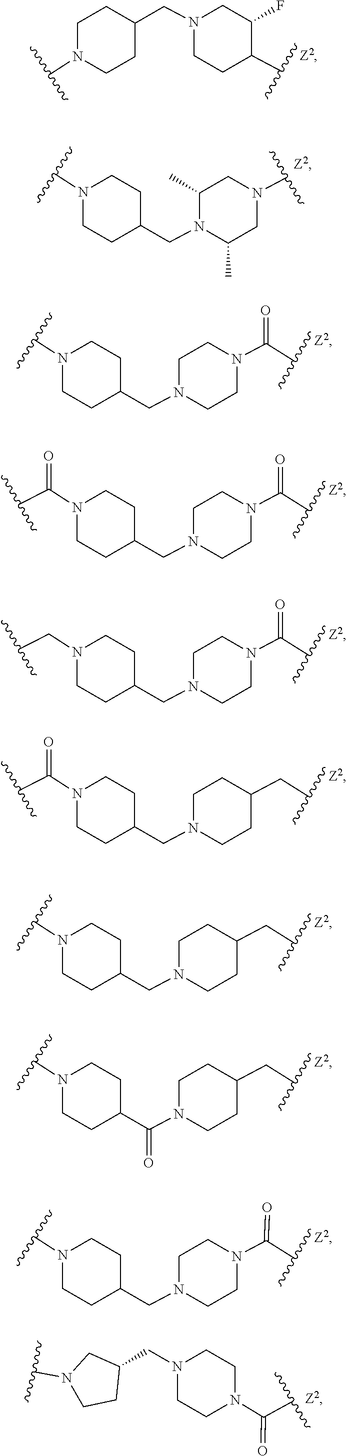 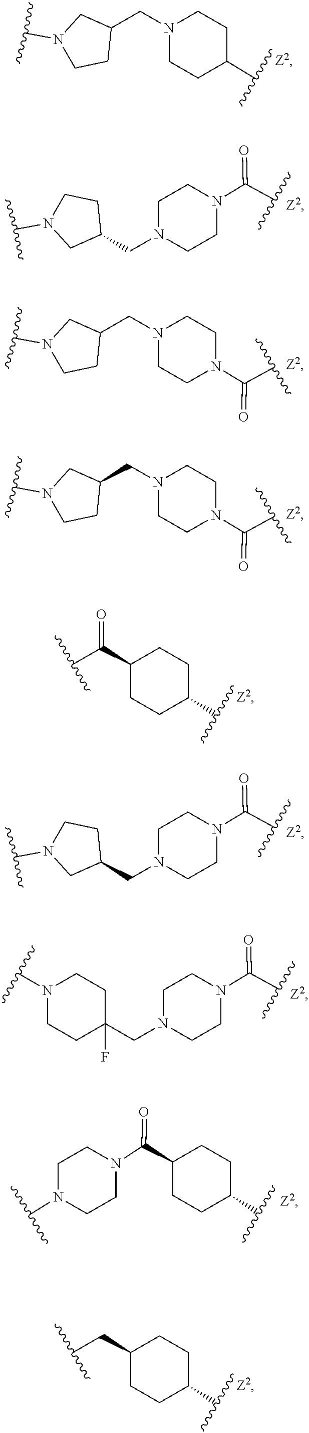 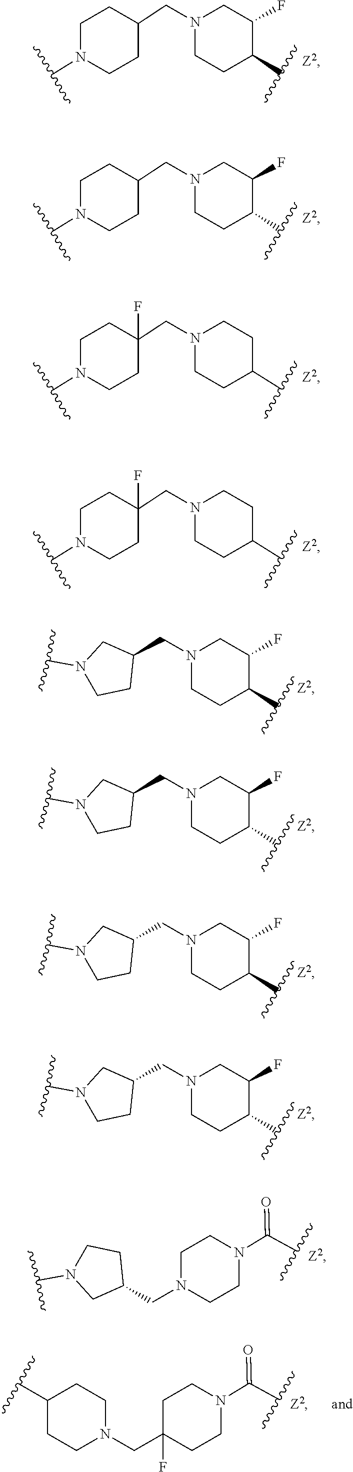 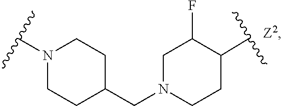 [SMILES: *C1CCN(CC2CCN(*)CC2)CC1F]
wherein [symbol] designates attachment to Z²; wherein X is oxygen, or sulfur.

50. The compound of Formula (I), having the following Formula (II): 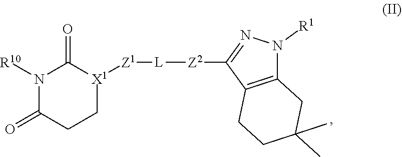
or stereoisomers and pharmaceutically acceptable salt(s) thereof.

51. The compound of Formula (I), having the following Formula (III): 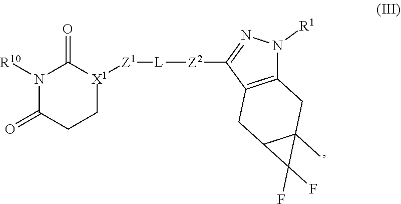
or stereoisomers and pharmaceutically acceptable salt(s) thereof.

52. The compound of Formula (III), having the following Formula (IIIa): 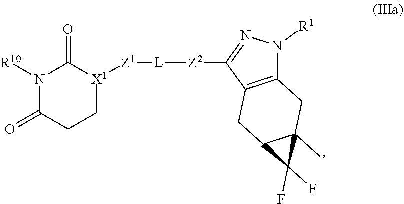
or stereoisomers and pharmaceutically acceptable salt(s) thereof.

53. The compound of Formula (I), having the following Formula (IV): 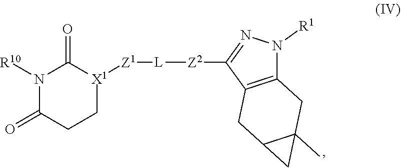
or stereoisomers and pharmaceutically acceptable salt(s) thereof.

54. The compound of Formula (IV), having the following Formula (IVa): 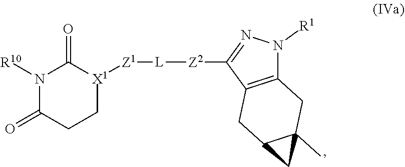
or stereoisomers and pharmaceutically acceptable salt(s) thereof.

55. The compound of Formula (I), having the following Formula (V): 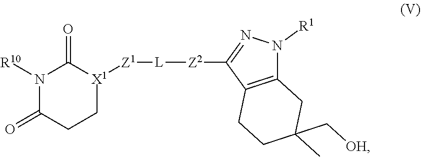
or stereoisomers and pharmaceutically acceptable salt(s) thereof.

56. The compound of Formula (V), having the following Formula (Va): 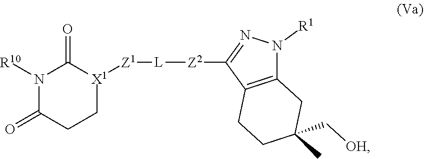
or stereoisomers and pharmaceutically acceptable salt(s) thereof.

57. The compound of Formula (V), having the following Formula (Vb): 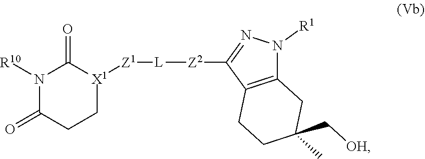
or stereoisomers and pharmaceutically acceptable salts thereof.

58. The compound of Formula (I), having the following Formula (VI): 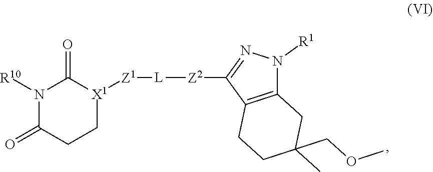
or stereoisomers and pharmaceutically acceptable salt(s) thereof.

59. The compound of Formula (VI), having the following Formula (VIa): 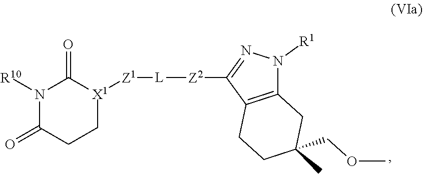
or stereoisomers and pharmaceutically acceptable salt(s) thereof.

60. The compound of Formula (VI), having the following Formula (VIb): 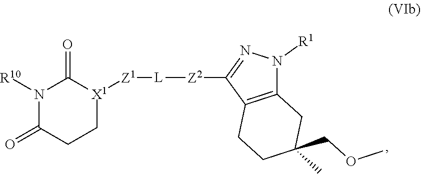
or stereoisomers and pharmaceutically acceptable salts thereof.

61. The compound of any of the previous claim wherein the compound is selected from the compounds in Table 1.

62. A pharmaceutical composition comprising the compound of any of the previous claims, or pharmaceutically acceptable carriers, excipients, or diluents.

63. A method of treating a disease or disorder in a subject in need thereof comprising the step of administering a therapeutically effective amount of the compound, or composition of any of the previous claims to the subject.

64. The method of claim 63 wherein the disease or disorder is cancer.

65. The compound or composition of any of the previous claims for use in therapy.

66. The compound, or composition of any of the previous claims for use in the treatment of cancer.
